(19) United States
(12) Patent Application Publication
(10) Pub. No.: US 2009/0298878 A1
(43) Pub. Date: Dec. 3, 2009

(54) OXIME COMPOUNDS AND THE USE THEREOF

(76) Inventors: Akira Matsumura, Osaka (JP); Hidenori Mikamiyama, Osaka (JP); Naoki Tsuno, Osaka (JP); Donald J. Kyle, Newton, PA (US); Bin Shao, Richboro, PA (US); Jiangchao Yao, Monmouth, NJ (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/373,328

(22) PCT Filed: Jul. 12, 2007
(86) PCT No.: PCT/US07/15827

§ 371 Date: Jan. 12, 2009

Related U.S. Application Data
(60) Provisional application No. 60/830,661, filed on Jul. 14, 2006.

(51) Int. Cl. A61K 31/445; C07D 211/98; G01N 23/00; C07D 401/12

(52) U.S. Cl. 514/318; 546/244; 514/315; 436/57; 546/194

(57) ABSTRACT

The invention relates to oxime compounds of Formula (I) and pharmaceutically acceptable salts, prodrugs, or solvates thereof, wherein X is hydrogen, optionally substituted aryl, optionally substituted heteroaryl or the like; Y is CO, SO₂, CR³R⁴ or the like; Z is optionally substituted lower alkyl, optionally substituted aryl or the like; W is optionally substituted lower alkylene or optionally substituted lower alkenylene, R³ and R⁴ are each independently hydrogen, lower alkyl or the like; p is 0, 1, or 2 and q is 0, 1 or 2. The invention is also directed to the use compounds of Formula I to treat, prevent or ameliorate a disorder responsive to the blockade of calcium channels, and particularly N-type calcium channels. Compounds of the present invention are especially useful for treating pain.

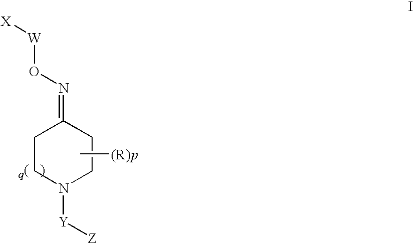

### BACKGROUND OF THE INVENTION

1. Field of the Invention

This invention is in the field of medicinal chemistry. The invention relates to oxime compounds and the discovery that these compounds act as blockers of calcium (Ca²⁺) channels.

2. Background Art

Calcium ions play fundamental roles in the regulation of many cellular processes. It is therefore essential that their intracellular levels be maintained under strict, yet dynamic control (Davila, H. M., Annals of the New York Academy of Sciences, pp. 102-117 (1999)). Voltage-gated calcium channels (VGCC) serve as one of the important mechanisms for fast calcium influx into the cell. Calcium channels are hetero-oligomeric proteins consisting of a pore-forming subunit (α1), which is able to form functional channels on its own in heterologous expression systems, and a set of auxiliary or regulatory subunits. Calcium channels have been classified based on their pharmacological and/or electrophysiological properties. The classification of voltage-gated calcium channels divides them into three groups: (i) high voltage-activated (HVA) channels, which include L-, N-, P-, and Q-types; (ii) intermediate (IVA) voltage-activated R-type channels; and (iii) low voltage-activated (LVA) T-type channels (Davila, supra). Voltage-gated calcium channels (VGCC) are also known as voltage-dependent calcium channels (VDCC) or voltage-sensitive calcium channels (VSCC).

Voltage-sensitive calcium channels (VSCC) regulate intracellular calcium concentration, which affects various important neuronal functions such as cellular excitability, neurotransmitter release, hormone secretion, intracellular metabolism, neurosecretory activity and gene expression (Hu et al., Bioorganic & Medicinal Chemistry 8:1203-1212 (2000)). N-type channels are found mainly in central and peripheral neurons, being primarily located on presynaptic nerve terminals. These channels regulate the calcium flux required for depolarization-evoked release of a transmitter from synaptic endings. The transmission of pain signals from the periphery to the central nervous system (CNS) is mediated by N-type calcium channels located in the spinal cord (Song et al., J. Med. Chem. 43:3474-3477 (2000)).

The six types of calcium channels (i.e., L, N, P, Q, R, and T) are expressed throughout the nervous system (Wallace, M. S., The Clinical Journal of Pain 16:580-585 (2000)). Voltage-sensitive calcium channels of the N-type exist in the superficial laminae of the dorsal horn and are thought to modulate nociceptive processing by a central mechanism. Blockade of the N-type calcium channel in the superficial dorsal horn modulates membrane excitability and inhibits neurotransmitter release, resulting in pain relief. Wallace (supra) suggests that based on animal models, N-type calcium channel antagonists have a greater analgesic potency than sodium channel antagonists. N-type calcium channel blockers have usefulness for neuroprotection and analgesia. Ziconotide, which is a selective N-type calcium channel blocker, has been found to have analgesic activity in animal models and neuroprotective activity in focal and global ischemia models (Song et al., supra). Examples of known calcium channel blockers include flunarizine, fluspirilene, cilnipide, PD 157767, SB-201823, SB-206284, NNC09-0026, and PD 151307 (Hu et al., supra).

Blockade of N-type channels can prevent and/or attenuate subjective pain as well as primary and/or secondary hyperalgesia and allodynia in a variety of experimental and clinical conditions (Vanegas, H. et al., Pain 85:9-18 (2000)). N-type voltage-gated calcium channels (VGCC) play a major role in the release of synaptic mediators such as glutamate, acetylcholine, dopamine, norepinephrine, gamma-aminobutyric acid (GABA) and calcitonin gene-related peptide (CGRP).

Inhibition of voltage-gated L-type calcium channels has been shown to be beneficial for neuroprotection (Song et al., supra). However, inhibition of cardiac L-type calcium channels can lead to hypotension. It is believed that a rapid and profound lowering of arterial pressure tends to counteract the neuroprotective effects of L-type calcium channel blockers. A need exists for antagonists that are selective for N-type calcium channels over L-type calcium channels to avoid potential hypotensive effects. Similar compounds to those of the present invention are described in WO2004/083 167, WO99/24399, JP50-084523, U.S. Pat. No. 3,577,441, WO 2005048933, U.S. Pat. No. 5,880,138, GB 2231048 and the like, but the structures of these compounds are different from those of the present invention.

### BRIEF SUMMARY OF THE INVENTION

The present invention is related to the use of oxime compounds represented by Formula I, I′, I″, II or II′, below, and the pharmaceutically acceptable salts, prodrugs and solvates thereof, as blockers of calcium (Ca²⁺) channels. Certain compounds of Formula I, I′, I″, II or II′ show selectivity as N-type calcium channel blockers. The invention is also related to treating, preventing or ameliorating a disorder responsive to the blockade of calcium channels in a mammal suffering from excess activity of said channels by administering an effective amount of a compound of Formula I, I′, I″, II or II′, or a pharmaceutically acceptable salt, prodrug or solvate thereof, as described herein. Specifically, the invention is related to treating, preventing or ameliorating a disorder responsive to the blockade of N-type calcium channels in a mammal suffering from excess activity of said channels by administering an effective amount of a compound of Formula I, I′, I″, II or II′, or a pharmaceutically acceptable salt, prodrug or solvate thereof, as described herein.

One aspect of the present invention is directed to novel compounds of Formula I or II, or a pharmaceutically acceptable salt, prodrug or solvate thereof.

Another aspect of the present invention is directed to the use of the novel compounds of Formula T. I″ or II or compounds of Formula I′ or II′, or a pharmaceutically acceptable salt, prodrug or solvate thereof, as blockers of N-type calcium channels. A further aspect of the present invention is to provide a method for treating, preventing or ameliorating stroke, neuronal damage resulting from head trauma, epilepsy, pain (e.g., acute pain, chronic pain, which includes but is not limited to, neuropathic pain and inflammatory pain, or surgical pain), migraine, a mood disorder, schizophrenia, a neurodegenerative disorder (e.g., Alzheimer's disease, amyotrophic lateral sclerosis (ALS), or Parkinson's disease), depression, anxiety, a psychosis, hypertension, or cardiac arrhythmia, by administering an effective amount of a compound of Formula I, I′, I″, II or II′, or a pharmaceutically acceptable salt, prodrug or solvate thereof, to a mammal in need of such treatment, prevention or amelioration.

A further aspect of the present invention is to provide a pharmaceutical composition useful for treating, preventing or ameliorating a disorder responsive to the blockade of calcium ion channels, especially N-type calcium ion channels, said pharmaceutical composition containing an effective amount of a compound of Formula I, I′, I″, II or II′, or a pharmaceutically acceptable salt, prodrug or solvate thereof, in a mixture with one or more pharmaceutically acceptable carriers.

Also, an aspect of the invention is to provide a method of modulating calcium channels, especially N-type calcium channels, in a mammal, wherein said method comprises administering to the mammal an effective amount of at least one compound of Formula I, I′, I″, II or II′, or a pharmaceutically acceptable salt, prodrug or solvate thereof.

A further aspect of the present invention is to provide radiolabeled compounds of Formula I, I′, I″, II or II′ and the use of such compounds, or their pharmaceutically acceptable salts, prodrugs or solvates, as radioligands for their binding site on the calcium channel.

A further aspect of the invention is to provide a method for screening a candidate compound for the ability to bind to a receptor using a ³H, ¹¹C or ¹⁴C radiolabeled compound of Formula I, I′, I″, II or II′, or a pharmaceutically acceptable salt, prodrug or solvate thereof. This method comprises a) introducing a fixed concentration of the radiolabeled compound to the receptor to form a mixture; b) titrating the mixture with a candidate compound; and c) determining the binding of the candidate compound to said receptor.

A further aspect of the invention is to provide the use of a compound of Formula I, I′, I″, II or II′, or a pharmaceutically acceptable salt, prodrug or solvate thereof, in the manufacture of a medicament for treating, preventing or ameliorating stroke, neuronal damage resulting from head trauma, epilepsy, pain, migraine, a mood disorder, schizophrenia, a neurodegenerative disorder, depression, anxiety, a psychosis, hypertension, or cardiac arrhythmia in a mammal. In a preferred embodiment, the invention provides the use of a compound of Formula I, I′, I″, II or II′, or a pharmaceutically acceptable salt, prodrug or solvate thereof, in the manufacture of a medicament for treating, preventing or ameliorating acute pain, chronic pain, or surgical pain.

Additional embodiments and advantages of the invention will be set forth in part in the description that follows, and will flow from the description, or may be learned by practice of the invention. The embodiments and advantages of the invention will be realized and attained by means of the elements and combinations particularly pointed out in the appended claims.

It is to be understood that both the foregoing summary and the following detailed description are exemplary and explanatory only and are not restrictive of the invention, as claimed.

### DETAILED DESCRIPTION OF THE INVENTION

One aspect of the present invention is based upon the use of compounds of Formula I, I′, I″, II or II′, and the pharmaceutically acceptable salts, prodrugs and solvates thereof, as blockers of Ca²⁺ channels. In view of this property, compounds of Formula I, I′, I″, II or II′, and the pharmaceutically acceptable salts, prodrugs and solvates thereof, are useful for treating disorders responsive to the blockade of calcium ion channels. In one aspect, compounds of Formula I, I′, I″, II or II′, and the pharmaceutically acceptable salts, prodrugs and solvates thereof, selectively block N-type calcium ion channels and, thus, are useful for treating disorders responsive to the selective blockade of N-type calcium ion channels.

The present invention provides

1) A compound having the Formula I:

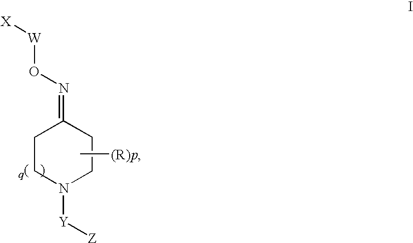

a pharmaceutically acceptable salt, a prodrug or a solvate thereof, wherein:
W is absent, optionally substituted lower alkylene or optionally substituted lower alkenylene,
X is optionally substituted aryl, optionally substituted heterocyclyl, optionally substituted cycloalkyl or optionally substituted cycloalkenyl and
When W is optionally substituted lower alkylene or optionally substituted lower alkenylene, then X can additionally be hydrogen;
Y is CO, SOₘ or CR³R⁴;

Z is optionally substituted lower alkyl, optionally substituted lower alkenyl, optionally substituted lower alkynyl, optionally substituted lower alkoxy, optionally substituted lower alkenyloxy, optionally substituted lower alkynyloxy, optionally substituted amino, optionally substituted lower alkylthio, optionally substituted lower alkenylthio, optionally substituted lower alkynylthio, optionally substituted cycloalkyl, optionally substituted cycloalkenyl, optionally substituted aryl or optionally substituted heterocyclyl,

when Y is CO, then Z can additionally be optionally substituted acyl, optionally substituted carbamoyl, optionally substituted cycloalkyloxy, optionally substituted cycloalkenyloxy, optionally substituted aryloxy or optionally substituted heterocyclyloxy,

when Y is CR³R⁴, then Z can additionally be hydrogen, hydroxy, halogen, carboxy, optionally substituted lower alkoxycarbonyl, optionally substituted lower alkylthio, optionally substituted acyl, optionally substituted acyloxy, optionally substituted lower alkylsulfonyl, optionally substituted lower alkylsulfonyloxy, optionally substituted lower alkylsulfinyl, optionally substituted carbamoyl, optionally substituted carbamoyloxy, optionally substituted sulfamoyl, optionally substituted sulfamoyloxy, optionally substituted aryloxy, optionally substituted aryloxycarbonyl, optionally substituted arylthio, optionally substituted arylsulfonyl, optionally substituted arylsulfinyl, optionally substituted arylsulfonyloxy, optionally substituted arylsulfinyloxy, optionally substituted heterocyclyloxy, optionally substituted heterocyclyloxycarbonyl, optionally substituted heterocyclylthio, optionally substituted heterocyclylsulfonyl, optionally substituted heterocyclylsulfinyl, optionally substituted heterocyclylsulfonyloxy, optionally substituted heterocyclylsulfinyloxy,
R³ and T⁴ are each independently hydrogen, halogen, lower alkyl, halo(lower)alkyl, hydroxy(lower)alkyl, amino, lower alkylamino, optionally substituted cycloalkyl, optionally substituted cycloalkenyl, optionally substituted aryl or optionally substituted heterocyclyl, or together with the carbon atom to which they are attached form optionally substituted carbocycle or optionally substituted heterocycle;
R is lower alkyl, hydroxy(lower)alkyl, lower alkoxy(lower)alkyl, carboxy, lower alkoxycarbonyl, carbamoyl or lower alkylcarbamoyl,
m is 1 or 2,
p is 0, 1 or2, and
q is 0, 1 or 2 with the following provisos:

i) when Y is SO₂, then Z is not lower alkyl substituted with at least one substituent selected from CONHOH, COOH and lower alkoxycarbonyl,

ii) Y-Z is not benzoyl, acetyl, carbamoyl or lower alkoxycarbonyl,

iii) when Y is CO, then Z is not methylene substituted with heterocyclidene, and

iv) when Y is SO₂, then —W—X is not 2-tetrahydrofuryl.

1′) A compound having the Formula II:

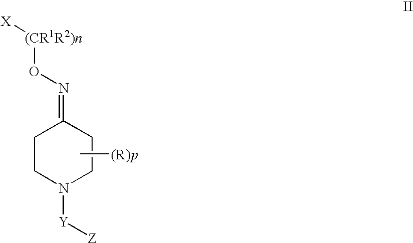

and pharmaceutically acceptable salts, prodrugs and solvates thereof, wherein:
X is optionally substituted aryl, optionally substituted heterocyclyl, optionally substituted cycloalkyl or optionally substituted cycloalkenyl;
Y is CO, SOₘ, or CR³R⁴;
Z is optionally substituted lower alkyl, optionally substituted lower alkenyl, optionally substituted lower alkynyl, optionally substituted lower alkoxy, optionally substituted lower alkenyloxy, optionally substituted lower alkynyloxy, optionally substituted amino, optionally substituted lower alkylthio, optionally substituted lower alkenylthio, optionally substituted lower alkynylthio, optionally substituted cycloalkyl, optionally substituted cycloalkenyl, optionally substituted aryl or optionally substituted heterocyclyl,

when Y is CO, then Z can additionally be optionally substituted acyl or optionally substituted carbamoyl,

when Y is CR³R⁴, then Z can additionally be hydrogen, hydroxy, halogen, optionally substituted lower alkoxy, carboxy, optionally substituted lower alkoxycarbonyl, optionally substituted lower alkylthio, optionally substituted acyl, optionally substituted acyloxy, optionally substituted lower alkylsulfonyl, optionally substituted lower alkylsulfonyloxy, optionally substituted lower alkylsulfinyl, optionally substituted carbamoyl, optionally substituted carbamoyloxy, optionally substituted sulfamoyl, optionally substituted sulfamoyloxy,

optionally substituted aryloxy, optionally substituted aryloxycarbonyl, optionally substituted arylthio, optionally substituted arylsulfonyl, optionally substituted arylsulfinyl, optionally substituted arylsulfonyloxy, optionally substituted arylsulfinyloxy, optionally substituted heterocyclyloxy, optionally substituted heterocyclyloxycarbonyl, optionally substituted heterocyclylthio, optionally substituted heterocyclylsulfonyl, optionally substituted heterocyclylsulfinyl, optionally substituted heterocyclylsulfonyloxy, optionally substituted heterocyclylsulfinyloxy,
R¹ and R² are each independently hydrogen, halogen, lower alkyl, halo(lower)alkyl, optionally substituted cycloalkyl, optionally substituted cycloalkenyl, optionally substituted aryl or optionally substituted heterocyclyl;
R³ and R⁴ are each independently hydrogen, halogen, lower alkyl, halo(lower)alkyl, hydroxy(lower)alkyl, amino, lower alkylamino, optionally substituted cycloalkyl, optionally substituted cycloalkenyl, optionally substituted aryl or optionally substituted heterocyclyl;
R is lower alkyl, hydroxy(lower)alkyl, lower alkoxy(lower)alkyl, carboxy, lower alkoxycarbonyl, carbamoyl or lower alkylcarbamoyl,
n is 0, 1, 2 or 3 and
m is 1 or 2,
p is 0, 1 or 2, with the following provisos:

i) when Y is SO₂, then Z is not lower alkyl substituted with at least one substituent selected from CONHOH, COOH and lower alkoxycarbonyl,

ii) Y-Z is not benzoyl, acetyl, carbamoyl or lower alkoxycarbonyl,

iii) when Y is CO, then Z is not methylene substituted with heterocyclidene, and

iv) when Y is SO₂, then X is not tetrahydrofuran.

2) The compounds of the above 1), wherein q is 1.

3) The compound of the above 1) or 2), wherein W is (CR¹R²)n, n is 0, 1, 2 or 3, R¹ and R² are each independently hydrogen, halogen, lower alkyl, halo(lower)alkyl, hydroxy(lower)alkyl, lower alkoxy(lower)alkyl, optionally substituted aryl(lower)alkyl, optionally substituted aryloxy(lower)alkyl, optionally substituted heterocyclyl(lower)alkyl, optionally substituted

heterocyclyloxy(lower)alkyl, optionally substituted cycloalkyl, optionally substituted cycloalkenyl, optionally substituted aryl or optionally substituted heterocyclyl and X is optionally substituted aryl, optionally substituted heterocyclyl, optionally substituted cycloalkyl or optionally substituted cycloalkenyl.

4) The compound of any of the above 1), 1′), 2) or 3), wherein W is optionally substituted methylene.

5) The compound of any of the above 1),1′) or 2) to 4), wherein X is optionally substituted phenyl or optionally substituted pyridyl.

6) The compound of any one of the above 1), 1′) or 2) to 5), wherein Y is CO or SO₂.

7) The compound of any one of the above 1), 1′) or 2) to 6), wherein Z is optionally substituted lower alkyl or optionally substituted phenyl.

8) A pharmaceutical composition, comprising the compound of any of the above 1), 1′) or 2) to 7) and a pharmaceutically acceptable carrier.

9) A method of treating, preventing or ameliorating a disorder responsive to the blockade of calcium channels in a mammal suffering from said disorder, comprising administering to a mammal in need of such treatment, prevention or amelioration an effective amount of a compound of any of the above 1), 1′) or 2) to 7).

10) The method of the above 9), wherein a disorder responsive to the blockade of N-type calcium channels is treated, prevented or ameliorated.

11) A method for treating, preventing or ameliorating stroke,neuronal damage resulting from head trauma, epilepsy, pain, migraine, a mood disorder, schizophrenia, a neurodegenerative disorder, depression, anxiety, a psychosis, hypertension or cardiac arrhythmia in a mammal, comprising administering an effective amount of a compound of any of the above 1), 1′) or 2) to 7).

12) The method of the above 11), wherein the method is for treating, preventing or ameliorating pain selected from chronic pain, acute pain, and surgical pain.

13) A method of modulating calcium channels in a mammal, comprising administering to the mammal at least one compound of any one of the above 1), 1′) or 2) to 7).

14) The method of the above 13), wherein the N-type calcium channel is modulated.

15) A compound having the Formula I as described in the above 1), 1′) or 2) to 7), wherein the compound is ³H, ¹¹C, or ¹⁴C radiolabeled.

16) A method of screening a candidate compound for the ability to bind to a receptor using a radiolabeled compound of the above 15), comprising a) introducing a fixed concentration of the radiolabeled compound to the receptor to form a mixture; b) titrating the mixture with a candidate compound; and c) determining the binding of the candidate compound to said receptor.

17) Use of a compound of Formula I as claimed in any one of the above 1), 1′) or 2) to 7) in the manufacture of a medicament for treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal.

18) Use of a compound of Formula I as claimed in any one of the above 1), 1′) or 2) to 7) in the manufacture of a medicament for treating, preventing or ameliorating pain selected from chronic pain, acute pain, and surgical pain.

19) A pharmaceutical composition for modulating calcium channels in a mammal, comprising the compound having the Formula I′:

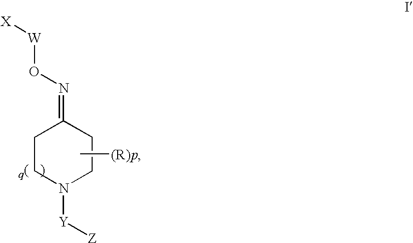

a pharmaceutically acceptable salt, a prodrug or a solvate thereof, wherein:
W is absent, optionally substituted lower alkylene or optionally substituted lower alkenylene,
X is optionally substituted aryl, optionally substituted heterocyclyl, optionally substituted cycloalkyl or optionally substituted cycloalkenyl and
When W is optionally substituted lower alkylene or optionally substituted lower alkenylene, then X can additionally be hydrogen;
Y is CO, SOₘ or CR³R⁴;
Z is optionally substituted lower alkyl, optionally substituted lower alkenyl, optionally substituted lower alkynyl, optionally substituted lower alkoxy, optionally substituted lower alkenyloxy, optionally substituted lower alkynyloxy, optionally substituted amino, optionally substituted lower alkylthio, optionally substituted lower alkenylthio, optionally substituted lower alkynylthio, optionally substituted cycloalkyl, optionally substituted cycloalkenyl, optionally substituted aryl or optionally substituted heterocyclyl,
when Y is CO, then Z can additionally be optionally substituted acyl, optionally substituted carbamoyl, optionally substituted cycloalkyloxy, optionally substituted cycloalkenyloxy, optionally substituted aryloxy or optionally substituted heterocyclyloxy,
when Y is CR³R⁴, then Z can additionally be hydrogen, hydroxy, halogen, carboxy, optionally substituted lower alkoxycarbonyl, optionally substituted lower alkylthio, optionally substituted acyl, optionally substituted acyloxy, optionally substituted lower alkylsulfonyl, optionally substituted lower alkylsulfonyloxy, optionally substituted lower alkylsulfinyl, optionally substituted carbamoyl, optionally substituted carbamoyloxy, optionally substituted sulfamoyl, optionally substituted sulfamoyloxy,
optionally substituted aryloxy, optionally substituted aryloxycarbonyl, optionally substituted arylthio, optionally substituted arylsulfonyl, optionally substituted arylsulfinyl, optionally substituted arylsulfonyloxy, optionally substituted arylsulfinyloxy, optionally substituted heterocyclyloxy, optionally substituted heterocyclyloxycarbonyl, optionally substituted heterocyclylthio, optionally substituted heterocyclylsulfonyl, optionally substituted heterocyclylsulfinyl, optionally substituted heterocyclylsulfonyloxy, optionally substituted heterocyclylsulfinyloxy,
when Z is optionally substituted cycloalkyl, optionally substituted cycloalkenyl, optionally substituted aryl or optionally substituted heterocyclyl, then Y can additionally be absent;

R³ and R⁴ are each independently hydrogen, halogen, lower alkyl, halo(lower)alkyl, hydroxy(lower)alkyl, amino, lower alkylamino, optionally substituted cycloalkyl, optionally substituted cycloalkenyl, optionally substituted aryl or optionally substituted heterocyclyl, or together with the carbon atom to which they are attached form optionally substituted carbocycle or optionally substituted heterocycle;
R is lower alkyl, hydroxy(lower)alkyl, lower alkoxy(lower)alkyl, carboxy, lower alkoxycarbonyl, carbamoyl or lower alkylcarbamoyl,
m is 1 or 2,
p is 0, 1 or 2, and
q is 0, 1 or 2

and a pharmaceutically acceptable carrier.

19′) A pharmaceutical composition for modulating calcium channels in a mammal, comprising the compound having the Formula II′:

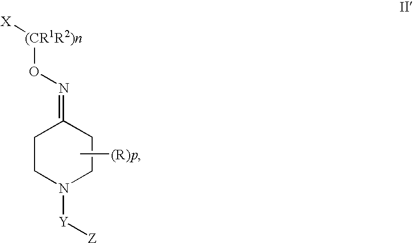

and pharmaceutically acceptable salts, prodrugs and solvates thereof, wherein:
X is optionally substituted aryl, optionally substituted heterocyclyl, optionally substituted cycloalkyl or optionally substituted cycloalkenyl;
Y is CO, SOₘ or CR³R⁴;
Z is optionally substituted lower alkyl, optionally substituted lower alkenyl, optionally substituted lower alkynyl, optionally substituted lower alkoxy, optionally substituted lower alkenyloxy,optionally substituted lower alkynyloxy, optionally substituted amino, optionally substituted lower alkylthio, optionally substituted lower alkenylthio, optionally substituted lower alkynylthio, optionally substituted cycloalkyl, optionally substituted cycloalkenyl, optionally substituted aryl or optionally substituted heterocyclyl,
when Y is CO, then Z can additionally be optionally substituted acyl or optionally substituted carbamoyl,
when Y is CR³R⁴, then Z can additionally be hydrogen, hydroxy, halogen, carboxy, optionally substituted lower alkoxycarbonyl, optionally substituted lower alkylthio, optionally substituted acyl, optionally substituted acyloxy, optionally substituted lower alkylsulfonyl, optionally substituted lower alkylsulfonyloxy, optionally substituted lower alkylsulfinyl, optionally substituted carbamoyl, optionally substituted carbamoyloxy, optionally substituted sulfamoyl, optionally substituted sulfamoyloxy, optionally substituted aryloxy, optionally substituted aryloxycarbonyl, optionally substituted arylthio, optionally substituted arylsulfonyl, optionally substituted arylsulfinyl, optionally substituted arylsulfonyloxy, optionally substituted arylsulfinyloxy, optionally substituted heterocyclyloxy, optionally substituted heterocyclyloxycarbonyl, optionally substituted heterocyclylthio, optionally substituted heterocyclylsulfonyl, optionally substituted heterocyclylsulfinyl, optionally substituted heterocyclylsulfonyloxy, optionally substituted heterocyclylsulfinyloxy, when Z is optionally substituted cycloalkyl, optionally substituted cycloalkenyl, optionally substituted aryl or optionally substituted heterocyclyl, then Y can additionally be absent;
R¹ and R² are each independently hydrogen, halogen, lower alkyl, halo(lower)alkyl, optionally substituted cycloalkyl, optionally substituted cycloalkenyl, optionally substituted aryl or optionally substituted heterocyclyl;
R³ and R⁴ are each independently hydrogen, halogen, lower alkyl, halo(lower)alkyl, hydroxy(lower)alkyl, amino, lower alkylamino, optionally substituted cycloalkyl, optionally substituted cycloalkenyl, optionally substituted aryl or optionally substituted heterocyclyl;
R is lower alkyl, hydroxy(lower)alkyl, lower alkoxy(lower)alkyl, carboxy, lower alkoxycarbonyl, carbamoyl or lower alkylcarbamoyl,
n is 0, 1, 2 or 3 and
m is 1 or 2,
p is 0, 1 or 2

and a pharmaceutically acceptable carrier.

20) The pharmaceutical composition of the above 19) or 19′), wherein q is 1.

21) The pharmaceutical composition of the above 19),19′) or 20), wherein W is (CR¹R²)n, n is 0, 1, 2 or 3, R¹ and R² are each independently hydrogen, halogen, lower alkyl, halo(lower)alkyl, hydroxy(lower)alkyl, lower alkoxy(lower)alkyl, optionally substituted aryl(lower)alkyl, optionally substituted aryloxy(lower)alkyl, optionally substituted heterocyclyl(lower)alkyl, optionally substituted heterocyclyloxy(lower)alkyl, optionally substituted cycloalkyl, optionally substituted cycloalkenyl, optionally substituted aryl or optionally substituted heterocyclyl and X is optionally substituted aryl, optionally substituted heterocyclyl, optionally substituted cycloalkyl or optionally substituted cycloalkenyl.

22) A method of treating, preventing or ameliorating a disorder responsive to the blockade of calcium channels in a mammal suffering from said disorder, comprising administering to a mammal in need of such treatment, prevention or amelioration an effective amount of a compound having the Formula I′ described in the above 19).

23) A method for treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal, comprising administering an effective amount of a compound having the Formula I′ described in the above 19).

24) The method of the above 23), wherein the method is for treating, preventing or ameliorating pain selected from chronic pain, acute pain, and surgical pain.

25) A method of modulating calcium channels in a mammal, comprising administering to the mammal at least one compound having the Formula I′ described in the above 19).

26) A method of screening a candidate compound for the ability to bind to a receptor using a ³H, ¹¹C, or ¹⁴C radiolabeled compound having the Formula I′ described in the above 19), comprising a) introducing a fixed concentration of the radiolabeled compound to the receptor to form a mixture; b) titrating the mixture with a candidate compound; and c) determining the binding of the candidate compound to said receptor.

27) Use of a compound having the Formula I′ described in the above 19) in the manufacture of a medicament for the treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal.

28) Use of a compound having the Formula I′ described in the above 19) in the manufacture of a medicament for the treating, preventing or ameliorating pain selected from chronic pain, acute pain, and surgical pain.

In the present specification, the term “halogen” includes fluorine, chlorine, bromine and iodine. Fluorine or chlorine is preferable. The halogen parts of “halo(lower)alkyl”, “halocycloalkyl”, “halocycloalkenyl”, “haloheterocyclyl”, “haloacyl” and “haloaryl” are the same as the above “halogen”.

The term “lower alkyl” includes straight or branched chain alkyl having 1 to 10 carbon atoms, preferably 1 to 6 carbon atoms and most preferably 1 to 3 carbon atoms. For example, included are methyl, ethyl, n-propyl, isopropyl, n-butyl, isobutyl, sec-butyl, tert-butyl, n-pentyl, isopentyl, neopentyl, hexyl, isohexyl, n-heptyl, isoheptyl, n-octyl, isooctyl, n-nonyl, n-decyl and the like.

The optional substituents in “optionally substituted lower alkyl”, include

1) halogen,

2) hydroxy,

3)carboxy,

4) mercapto,

5) cyano,

6) nitro,

7) lower alkoxy optionally substituted with at least one substituent selected from the group consisting of Group A and Group C,

8) acyl optionally substituted with at least one substituent selected form the group consisting of Group A, Group B and Group C,

9) acyloxy optionally substituted with at least one substituent selected form the group consisting of Group A, Group B and Group C

10) lower alkoxycarbonyl optionally substituted with at least one substituent selected form the group consisting of Group A and Group C,

11) aryloxycarbonyl optionally substituted with at least one substituent selected form the group consisting of Group A, Group B and Group C,

12) lower alkylthio optionally substituted with at least one substituent selected form the group consisting of Group A and Group C,

13) lower alkylsulfonyl optionally substituted with at least one substituent selected form the group consisting of Group A and Group C,

14) amino optionally substituted with at least one substituent selected form the group consisting of Group A, Group B and Group C

15) imino optionally substituted with at least one substituent selected form the group consisting of Group A, Group B and Group C,

16) carbamoyl optionally substituted with at least one substituent selected form the group consisting of Group A, Group B and Group C,

17) carbamoyloxy optionally substituted with at least one substituent selected form the group consisting of Group A, Group B and Group C,

18) thiocarbamoyl optionally substituted with at least one substituent selected form the group consisting of Group A, Group B and Group C,

19) cycloalkyl optionally substituted with at least one substituent selected form the group consisting of Group A, Group B and Group C,

20) cycloalkenyl optionally substituted with at least one substituent selected form the group consisting of Group A, Group B and Group C,

21) aryl optionally substituted with at least one substituent selected form the group consisting of Group A, Group B and Group C,

22) heterocyclyl optionally substituted with at least one substituent selected form the group consisting of Group A, Group B, Group C and oxo,

23) aryloxy optionally substituted with at least one substituent selected form the group consisting of Group A, Group B and Group C,

24) arylthio optionally substituted with at least one substituent selected form the group consisting of Group A, Group B and Group C,

25) arylsulfonyl optionally substituted with at least one substituent selected form the group consisting of Group A, Group B and Group C,

26) heterocyclylsulfonyl optionally substituted with at least one substituent selected form the group consisting of Group A, Group B, Group C, and oxo

and the like.

Group A includes hydroxy, halogen, cyano, nitro, lower alkoxy, halo(lower)alkoxy, hydroxy(lower)alkoxy, aryl(lower)alkoxy, acyl, haloacyl, aminoacyl, acyloxy, carboxy, lower alkoxycarbonyl, carbamoyl, lower alkylcarbamoyl, and optionally substituted amino, wherein the substituents are selected from the group consisting of halogen, hydroxy, lower alkyl,hydroxy(lower)alkyl, lower alkoxy(lower)alkyl, acyl, cycloalkyl, aryl and heterocyclyl.

Group B includes lower alkyl, halo(lower)alkyl, hydroxy(lower)alkyl, lower alkoxy(lower)alkyl, amino(lower)alkyl, lower alkylamino(lower)alkyl, cycloalkyl(lower)alkyl, aryl(lower)alkyl, halogenoaryl(lower)alkyl, hydroxyaryl(lower)alkyl, heterocyclyl(lower)alkyl, halogenoheterocyclyl(lower)alkyl and hydroxyaryl(lower)alkyl.

Group C includes optionally substituted cycloalkyl, optionally substituted cycloalkenyl, optionally substituted aryl and optionally substituted heterocyclyl, wherein the substituents are selected from the group consisting of Group A, Group B and oxo. The term “optionally substituted lower alkyl” refers to a lower alkyl that can be substituted with one or more of the above-mentioned substituents at any possible positions.

The lower alkyl parts of “lower alkoxy”, “lower alkoxycarbonyl”, “lower alkylsulfonyl”, “lower alkylsulfonyloxy”, “lower alkylsulfinyl”, “lower alkylthio”, “halo(lower)alkyl”, “hydroxy(lower)alkyl”, “amino(lower)alkyl”, “lower alkylamino(lower)alkyl”, “aryl(lower)alkyl”, “aryloxy(lower)alkyl”, “halo(lower)alkoxy”, “hydroxy(lower)alkoxy”, “lower alkoxy(lower)alkyl”, “lower alkoxy(lower)alkoxy”, “aryl(lower)alkoxy”, “lower alkylamino”, “hydroxy(lower)alkylamino”, “lower alkylcarbamoyl”, “lower alkylcarbamoyloxy”, “lower alkylslulfamoyl”, “lower alkylsulfamoyloxy”, “heterocyclyl(lower)alkyl”, “heterocyclyloxy(lower)alkyl”, “lower alkylenedioxy” and “lower alkylene” are as defined for “lower alkyl”.

The optional substituents in “optionally substituted lower alkoxy”, “optionally substituted lower alkoxycarbonyl”, “optionally substituted lower alkylsulfonyl”, “optionally substituted lower alkylsulfonyloxy”, “optionally substituted lower alkylsulfinyl” and “optionally substituted lower alkylthio” include those defined for “optionally substituted lower alkyl”.

The optional substituents in “optionally substituted lower alkylene” include halogen, lower alkyl, halo(lower)alkyl, hydroxy(lower)alkyl, lower alkoxy(lower)alkyl, optionally substituted aryl(lower)alkyl, optionally substituted aryloxy(lower)alkyl, optionally substituted heterocyclyl(lower)alkyl, optionally substituted heterocyclyloxy(lower)alkyl, optionally substituted cycloalkyl, optionally substituted cycloalkenyl, optionally substituted aryl or optionally substituted heterocyclyl and the like.

The term “lower alkenyl” refers to straight or branched chain alkenyl of 2 to 10 carbon atoms, preferably 2 to 8 carbon atoms, more preferably 3 to 6 carbon atoms having at least one double bond at any possible positions. For example, useful lower alkenyl groups include vinyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl and the like.

The optional substituents in “optionally substituted lower alkenyl” include those defined for “optionally substituted lower alkyl”.

The lower alkenyl parts of “lower alkenyloxy”, “lower alkenylthio” and “lower alkenylene” are the same as the above “lower alkenyl”. The optional substituents in “optionally substituted lower alkenyl”, “optionally substituted lower alkenyloxy” and “optionally substituted lower alkenylthio” include those defined for “optionally substituted lower alkyl”.

The optional substituents in “optionally substituted lower alkenylene” include halogen, lower alkyl, halo(lower)alkyl, hydroxy(lower)alkyl, lower alkoxy(lower)alkyl, optionally substituted aryl(lower)alkyl, optionally substituted aryloxy(lower)alkyl, optionally substituted heterocyclyl(lower)alkyl, optionally substituted heterocyclyloxy(lower)alkyl, optionally substituted cycloalkyl, optionally substituted cycloalkenyl, optionally substituted aryl or optionally substituted heterocyclyl and X is optionally substituted aryl, optionally substituted heterocyclyl, optionally substituted cycloalkyl, optionally substituted cycloalkenyl and the like.

The term “acyl” refers to straight or branched chain aliphatic acyl having 1 to I 0 carbon atoms, preferably 1 to 6 carbon atoms, more preferably 1 to 4 carbon atoms, cyclic aliphatic acyl having 4 to 9 carbon atoms, preferably 4 to 7 carbon atoms, aroyl and heterocyclylcarbonyl. Suitable acyl groups include, for example, formyl, acetyl, propionyl, butyryl, isobutyryl, valeryl, pivaloyl, hexanoyl, acryloyl, propioloyl, methacryloyl, crotonoyl, cyclopropylcarbonyl, cyclohexylcarbonyl, cyclooctylcarbonyl, benzoyl, pyridinecarbonyl, pyrimidinecarbonyl, piperidincarbonyl, piperazinocarbonyl, morphorinocarbonyl and the like.

The optional substituents in “optionally substituted acyl” include those defined for “optionally substituted lower alkyl.”

The optional substituents for aroyl or heterocyclylcarbonyl are

1) lower alkyl optionally substituted with at least one substituent from Group B,

2) the same as those defined for “optionally substituted lower alkyl” and the like.

The acyl parts in “acyloxy” “haloacyl” and “aminoacyl” are those defined for “acyl”.

The optional substituents in “optionally substituted acyloxy” include those defined for “optionally substituted acyl.”

The term “carbocycle” refers to a carbocycle having 3 to 6 carbon atoms and includes cyclopropyl, cyclobutyl, cyclopentyl, cyclohexyl and the like.

The term “cycloalkyl” refers to a carbocycle having 3 to 6 carbon atoms and includes cyclopropyl, cyclobutyl, cyclopentyl, cyclohexyl and the like. Cycloalkyl can be fused with one or more carbocycles.

The cycloalkyl parts in “hydroxycycloalkyl” and “halocycloalkyl” are those defined for “cycloalkyl”.

The term “cycloalkenyl” refers to a group having at least one double bond at any possible positions in the above “cycloalkyl”. Cycloalkenyl can be fused with one or more carbocycles and/or aryl. Examples are cyclopropenyl, cyclobutenyl, cyclopentenyl, cyclohexenyl, cyclohexadienyl and fluorenyl.

The optional substituents in “optionally substituted carbocycle”, “optionally substituted cycloalkyl” and “optionally substituted cycloalkenyl” are

1) lower alkyl optionally substituted with at least one substituent selected from the group consisting of Group A and Group C, and

2) the same as those defined for “optionally substituted lower alkyl”.

The terms “optionally substituted carbocycle”, “optionally substituted cycloalkyl” and “optionally substituted cycloalkenyl” refers to “carbocycle”, “cycloalkyl” and “cycloalkenyl” defined above that can be substituted with one or more of these substituents, respectively.

The optional substituents in “optionally substituted amino”, exemplified are

1) lower alkyl optionally substituted with at least one substituent selected from the group consisting of Group A and Group C, and

2) the same as those defined for “optionally substituted lower alkyl”.

The term “lower alkylamino” refers to mono-alkylamino and di-alkylamino.

The optional substituents in “optionally substituted carbamoyl” are

1) lower alkyl optionally substituted with at least one substituent selected from the group consisting of Group A and Group C,

2) Group B and

3) Group C.

The optional substituents in “optionally substituted carbamoyloxy” are those defined for “optionally substituted carbamoyl”.

The optional substituents in “optionally substituted sulfamoyl” or “optionally substituted sulfamoyloxy” are those defined for “optionally substituted carbamoyl”.

The term “aryl” includes phenyl, naphthyl, anthryl, phenanthryl, indenyl and the like. Phenyl is preferable.

The aryl parts of “aryloxy”, “aryloxycarbonyl”, “arylthio”, “arylsulfonyl”,

“arylsulfinyl”, “arylsulfonyloxy”, “arylsulfinyloxy” “aryl(lower)alkyl”, “aryloxy(lower)alkyl”,“aryl(lower)alkoxy”, “hydroxyaryl” and “haloaryl” are the same as the above “aryl”.

The term “heterocyclyl” or “heterocycle” refers to a heterocyclic group containing at least one heteroatom arbitrarily selected from the group of O, S and N. Suitable heterocyclyl groups are, for example, 5- or 6-membered heteroaryl groups such as pyrrolyl, imidazolyl, pyrazolyl, pyridyl, pyridazinyl, pyrimidinyl, oxazolyl, isoxazolyl, oxadiazolyl, isothiazolyl, thiazolyl, thiadiazolyl, furyl and thienyl; fused heterocyclyl groups having two rings, such as indolyl, isoindolyl, indazolyl, indolizinyl, indolinyl, isoindolinyl, quinolyl, isoquinolyl, cinnolinyl, phthalazinyl, quinazolinyl, naphthyridinyl, quinoxalinyl, purinyl, pteridinyl, benzopyranyl, benzimidazolyl, benzisoxazolyl, benzoxazolyl benzoxadiazolyl, benzisothiazolyl, benzothiazolyl, benzothiadiazolyl, benzofuryl, isobenzofuryl, benzothienyl, benzotriazolyl, imidazopyridyl, triazoropyridyl, imidazothiazolyl, pyrazinopyridazinyl, quinazolinyl, quinolyl, isoquinolyl, naphthyridinyl, benzomorpholinyl, benzomorpholino, benzodioxolanyl, tetrahydroquinolyl and tetrahydrobenzothienyl; fused heterocyclyl groups having three rings such as carbazolyl, acridinyl, xanthenyl, phenothiazinyl, phenoxathiinyl, phenoxazinyl and dibenzofuryl; and non-aromatic heterocyclyl such as dioxanyl, thiiranyl, oxiranyl, oxathiolanyl, azetidinyl, thianyl, pyrrolidinyl, pyrrolinyl, imidazolidinyl, imidazolinyl, pyrazolidinyl, pyrazolinyl, piperidyl, piperazinyl, morpholinyl, morpholino, thiomorpholinyl, thiomorpholino, dihydropyridyl, dihydroisoxazolyl, tetrahydrofuryl, tetrahydropyranyl, tetrahydrothiazolyl and tetrahydroisothiazolyl.

The heterocyclyl parts of “heterocyclyloxy”, “heterocyclyl(lower)alkyl”, “heterocyclyloxy(lower)alkyl”, “heterocyclyloxycarbonyl”, “heterocyclylthio”, “heterocyclylsulfonyl”, “heterocyclylsulfinyl”, “heterocyclylsulfonyloxy”, “heterocyclylsulfinyloxy”“hydroxyheterocyclyl”, “haloheterocyclyl” and “oxoheterocyclyl” are the same as the above “heterocyclyl”.

The optional substituents in “optionally substituted aryl”, “optionally substituted aryloxy”, “optionally substituted aryloxycarbonyl”, “optionally substituted arylthio”, “optionally substituted arylsulfony”, “optionally substituted arylsulfinyl”, “optionally substituted arylsulfonyloxy”, “optionally substituted arylsulfonyloxy”, “optionally substituted aryl(lower)alkyl”, “optionally substituted aryloxy(lower)alkyl”, “optionally substituted heterocycle”, “optionally substituted heterocyclyl”, “optionally substituted heterocyclyl(lower)alkyl”, “optionally substituted heterocyclyloxy(lower)alkyl”, “optionally substituted heterocyclyloxy”, “optionally substituted heterocyclyloxycarbonyl”, “optionally substituted heterocyclylthio”, “optionally substituted heterocyclylsulfonyl”, “optionally substituted heterocyclylsulfinyl”, “optionally substituted heterocyclylsulfonyloxy”, “optionally substituted heterocyclylsulfinyloxy”, “optionally substituted phenyl” and “optionally substituted pyridyl” are

1) the same as those defined for “optionally substituted lower alkyl”,

2) lower alkyl optionally substituted with at least one substituent selected from the Group A and Group C,

3) oxo and

4) lower alkylenedioxy.

These substituents can attach to one or more of any possible positions.

The term “heterocyclidene” includes a divalent heterocyclyl group which can be induced by removing two hydrogens on the same carbon atom. Heterocycle part of the “heterocyclidene” is the same as the above “heterocyclyl”. Examples of “heterocyclidene” is tetrahydrobenzazepinylidene and the like.

In the compound of Formula I or I′, Z includes the followings.

1) when Y is CO, then

Z is optionally substituted lower alkyl, optionally substituted lower alkenyl, optionally substituted lower alkynyl, optionally substituted lower alkoxy, optionally substituted lower alkenyloxy, optionally substituted lower alkynyloxy, optionally substituted amino, optionally substituted lower alkylthio, optionally substituted lower alkenylthio, optionally substituted lower alkynylthio, optionally substituted cycloalkyl, optionally substituted cycloalkenyl, optionally substituted aryl, optionally substituted heterocyclyl, optionally substituted acyl, optionally substituted carbamoyl, optionally substituted cycloalkyloxy, optionally substituted cycloalkenyloxy, optionally substituted aryloxy or optionally substituted heterocyclyloxy,

2) when Y is SOm, then

Z is optionally substituted lower alkyl, optionally substituted lower alkenyl, optionally substituted lower alkynyl, optionally substituted lower alkoxy, optionally substituted lower alkenyloxy, optionally substituted lower alkynyloxy, optionally substituted amino, optionally substituted lower alkylthio, optionally substituted lower alkenylthio, optionally substituted lower alkynylthio, optionally substituted cycloalkyl, optionally substituted cycloalkenyl, optionally substituted aryl, or optionally substituted heterocyclyl,

3) when Y is CR³R⁴, then

Z is optionally substituted lower alkyl, optionally substituted lower alkenyl, optionally substituted lower alkynyl, optionally substituted lower alkoxy, optionally substituted lower alkenyloxy, optionally substituted lower alkynyloxy, optionally substituted amino, optionally substituted lower alkylthio, optionally substituted lower alkenylthio, optionally substituted lower alkynylthio, optionally substituted cycloalkyl, optionally substituted cycloalkenyl, optionally substituted aryl, optionally substituted heterocyclyl, hydrogen, hydroxy, halogen, carboxy, optionally substituted lower alkoxycarbonyl, optionally substituted lower alkylthio, optionally substituted acyl, optionally substituted acyloxy, optionally substituted lower alkylsulfonyl, optionally substituted lower alkylsulfonyloxy, optionally substituted lower alkylsulfinyl, optionally substituted carbamoyl, optionally substituted carbamoyloxy, optionally substituted sulfamoyl, optionally substituted sulfamoyloxy, optionally substituted aryloxy, optionally substituted aryloxycarbonyl, optionally substituted arylthio, optionally substituted arylsulfonyl, optionally substituted arylsulfinyl, optionally substituted arylsulfonyloxy, optionally substituted arylsulfinyloxy, optionally substituted heterocyclyloxy, optionally substituted heterocyclyloxycarbonyl, optionally substituted heterocyclylthio, optionally substituted heterocyclylsulfonyl, optionally substituted heterocyclylsulfinyl, optionally substituted heterocyclylsulfonyloxy or optionally substituted heterocyclylsulfinyloxy.

The compound of the present invention includes the solvate thereof and hydrate is preferable. An example of the solvate is a solvate with an organic solvent and/or water. The compound of the present invention may cooperate with arbitrary numbers of water molecules to give a hydrate thereof.

The phrase “when Y is SO₂, then Z is not lower alkyl substituted with at least one substituent selected from CONHOH, COOH and lower alkoxycarbonyl” means the exclusion of the compound wherein Y is SO₂ and Z is lower alkyl substituted with CONHOH, COOH and/or lower alkoxycarbonyl. In this case, the lower alkyl of Z can be substituted with additional substituents.

Illustrative Oxime Compounds are the compounds of the following formula I″:

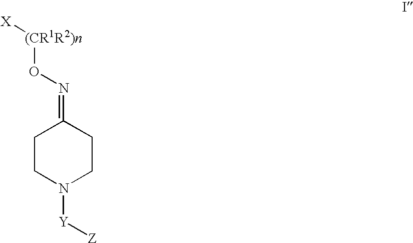

wherein X is one of the followings:

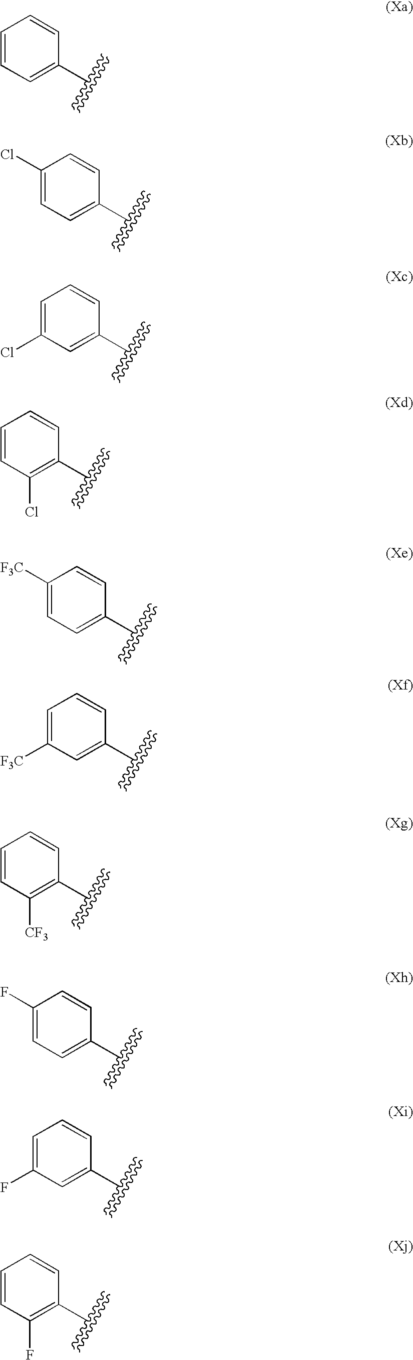

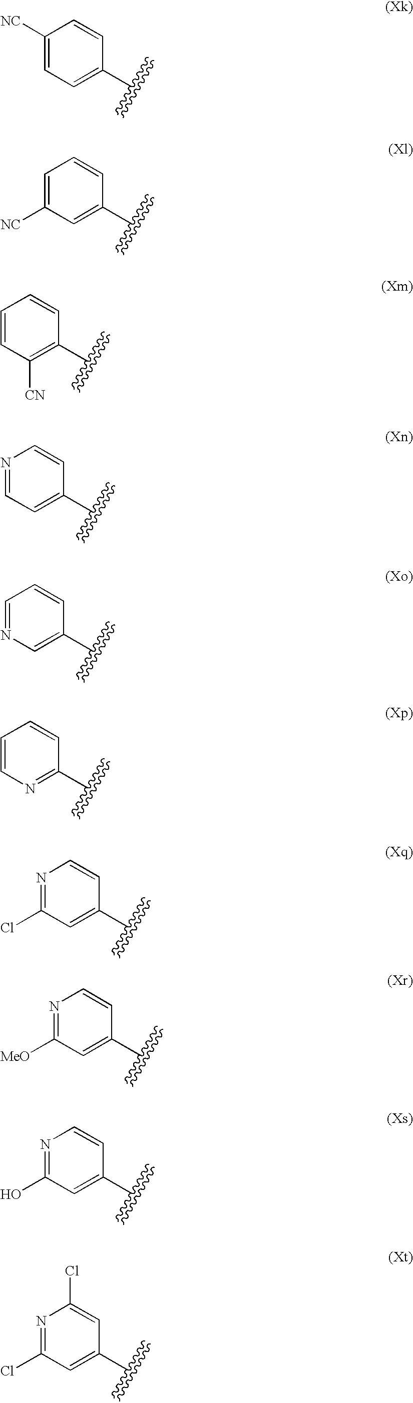

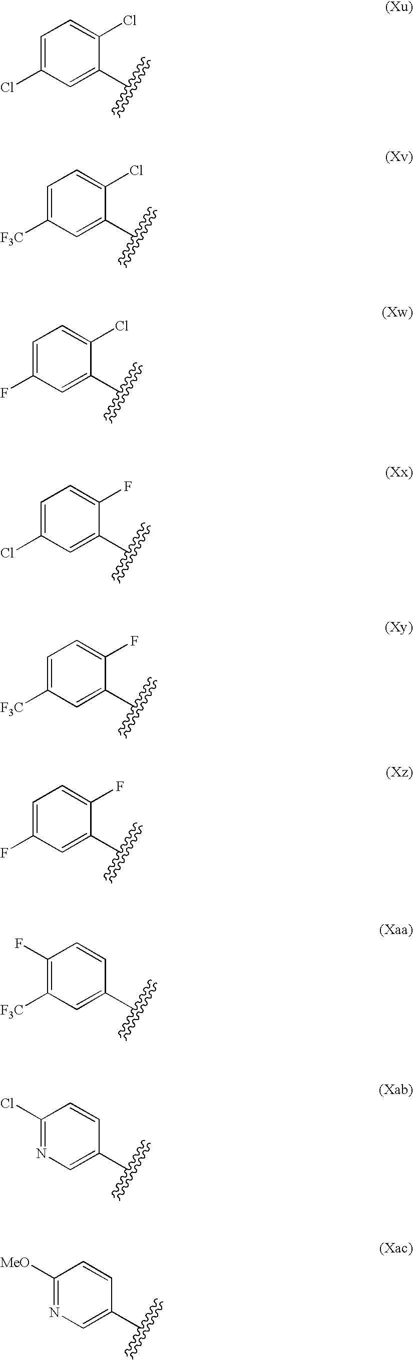

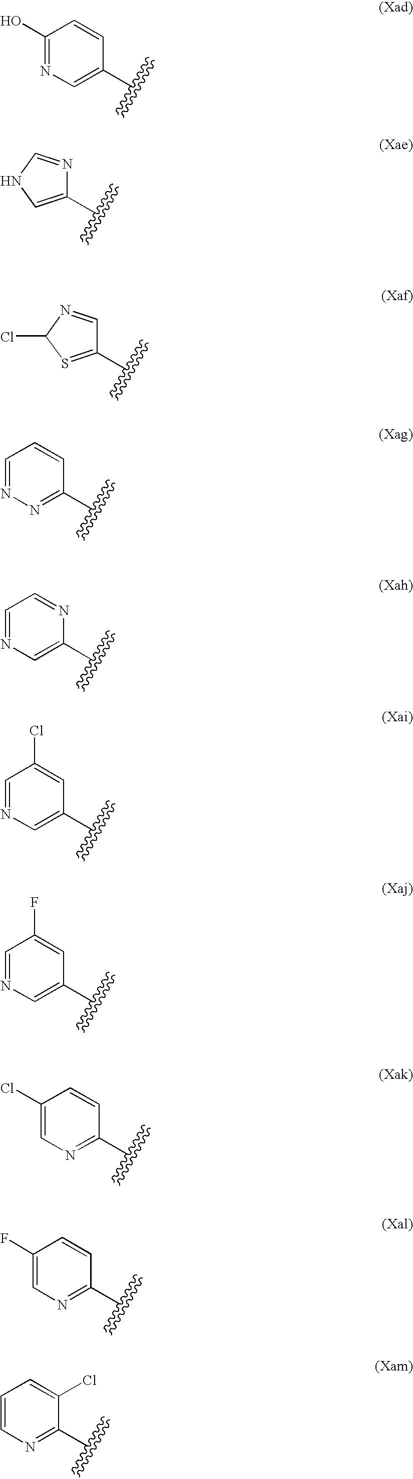

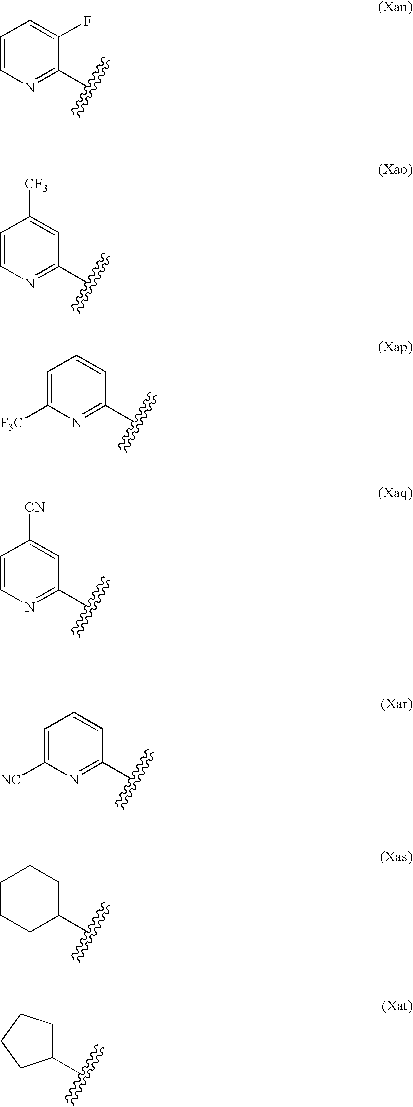

In another embodiment, the compounds of the formula I″ wherein (CR¹R²)n is absent (hereinafter referred to as CR¹R² is Ca), CH₂ (hereinafter referred to as CR¹R² is Cb), CHMe (hereinafter referred to as CR¹R² is Cc), CMe₂ (hereinafter referred to as CR¹R² is Cd), CH₂CH₂ (hereinafter referred to as CR¹R² is Ce) or CHPh (hereinafter referred to as CR¹R² is Cf).

In another embodiment, the compounds of the formula I″ wherein Y-Z is one of the followings:

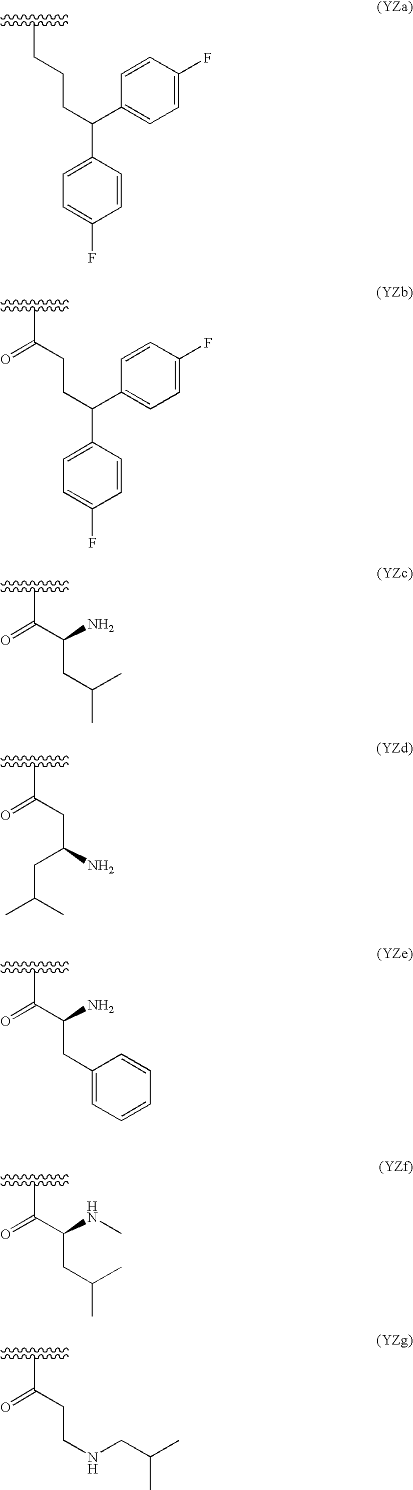

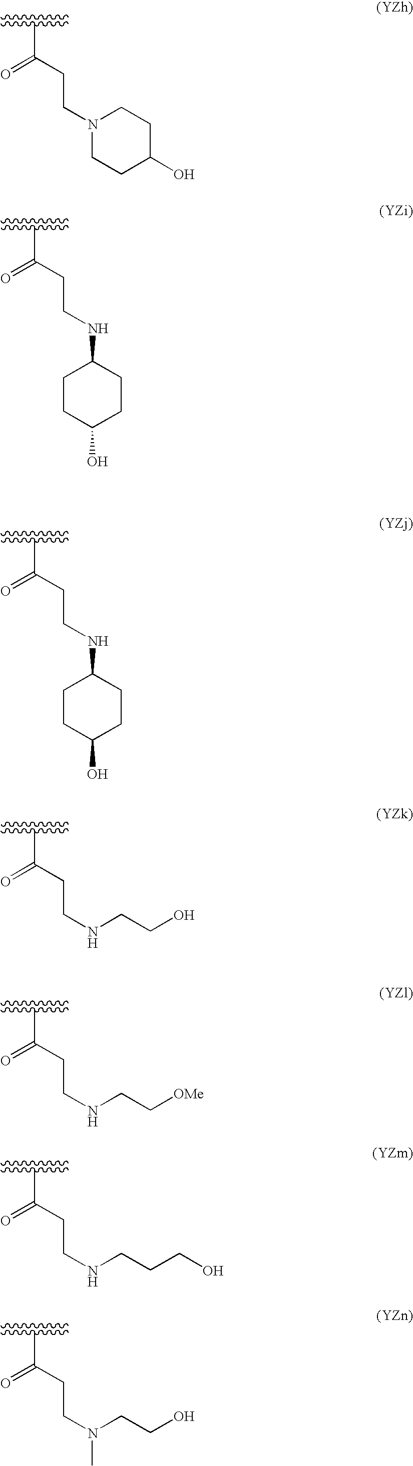

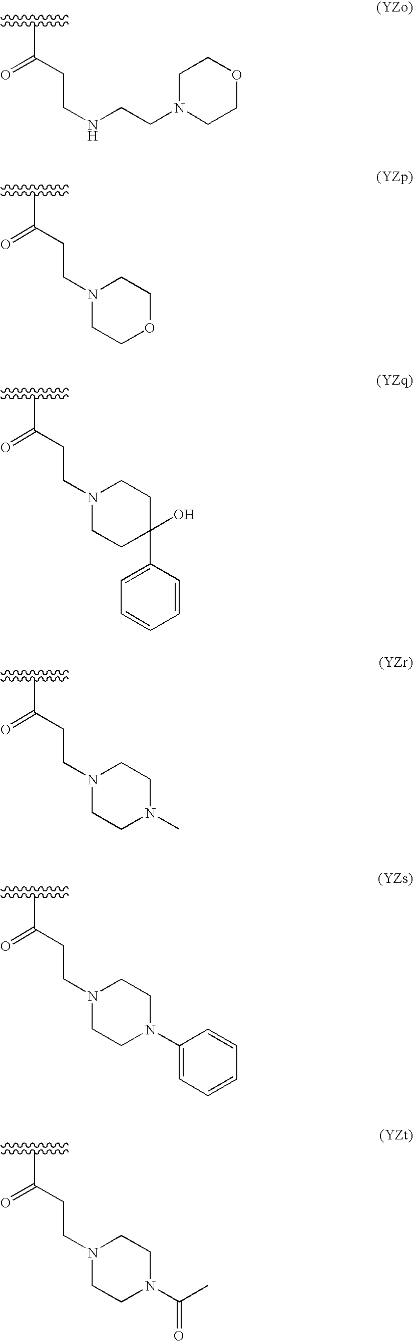

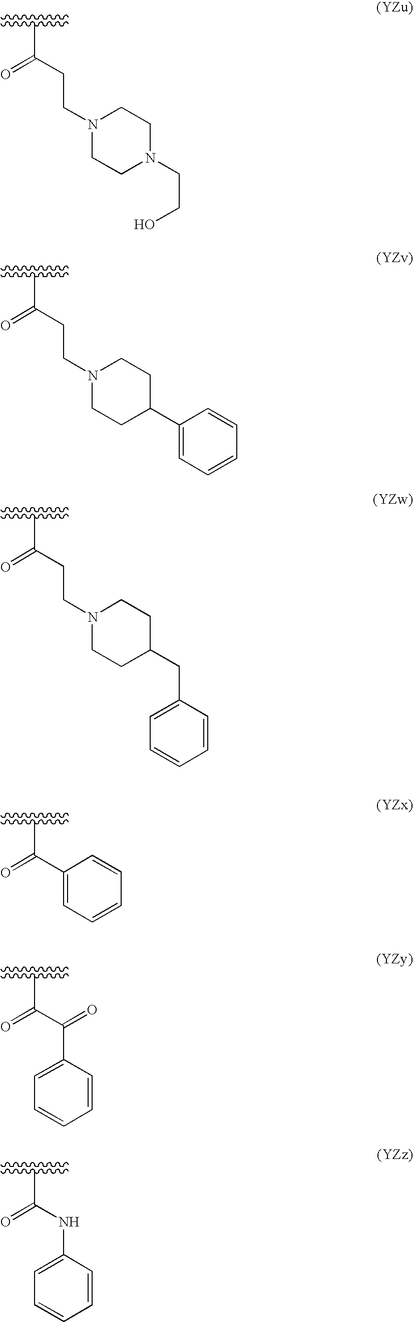

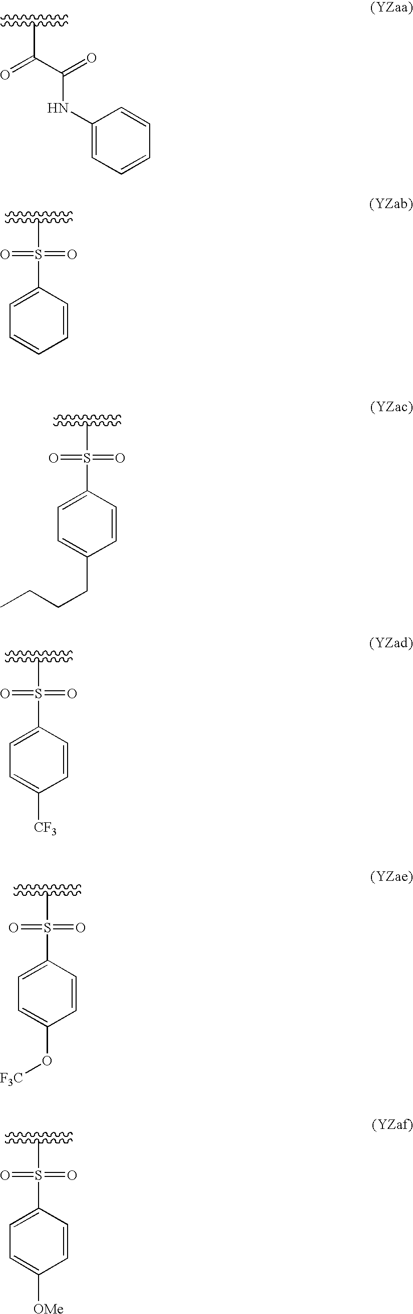

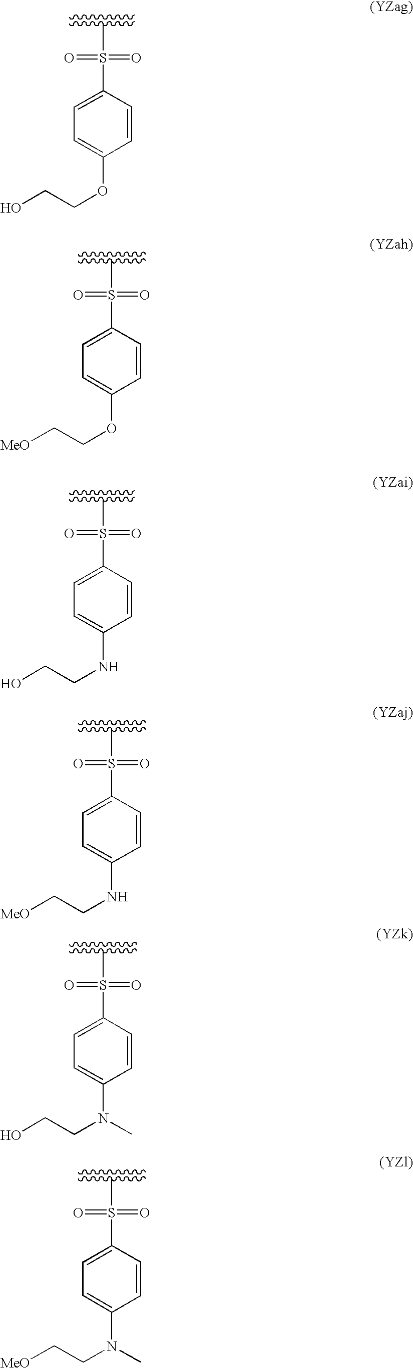

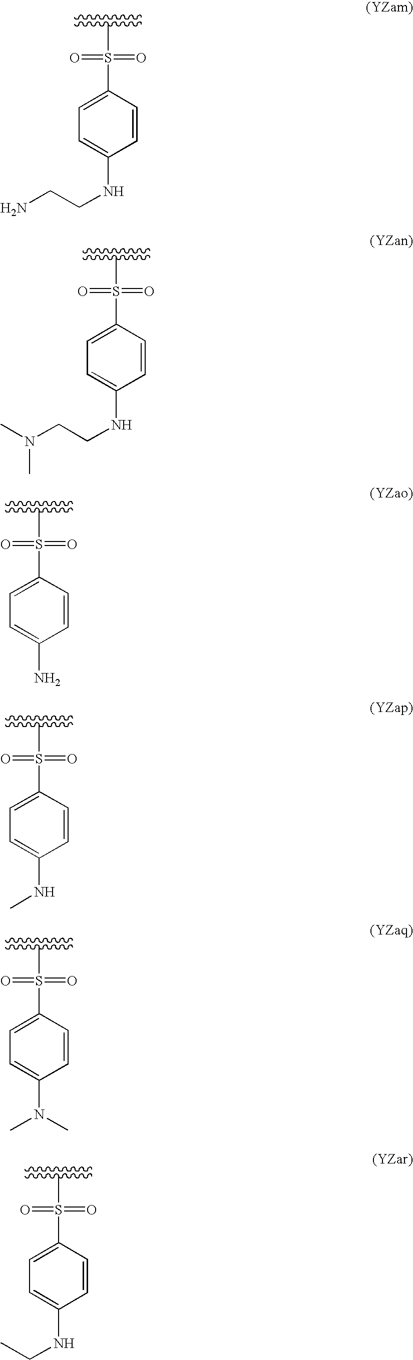

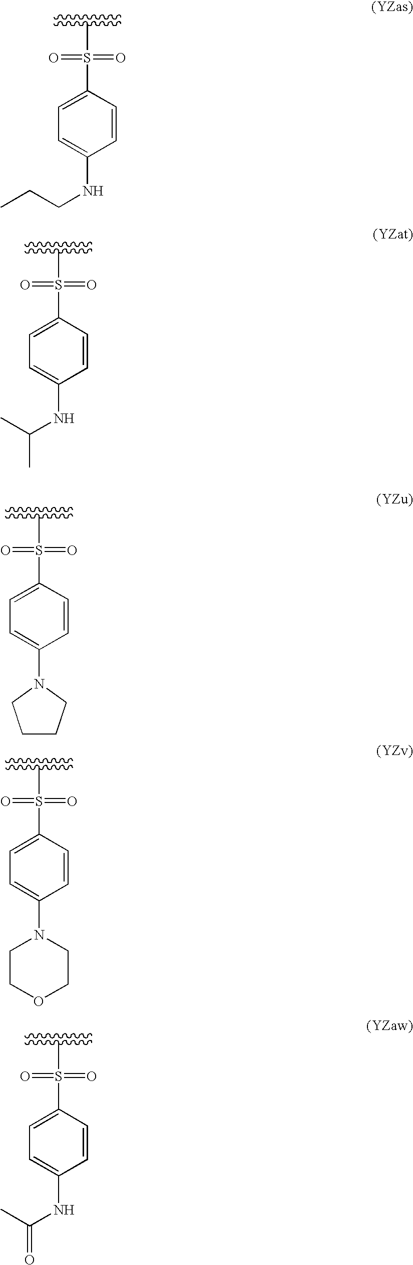

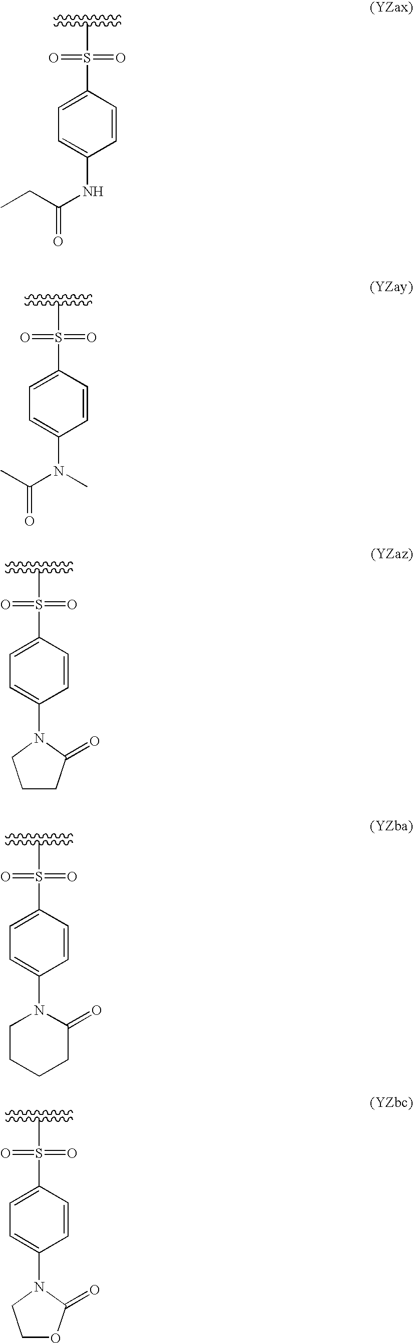

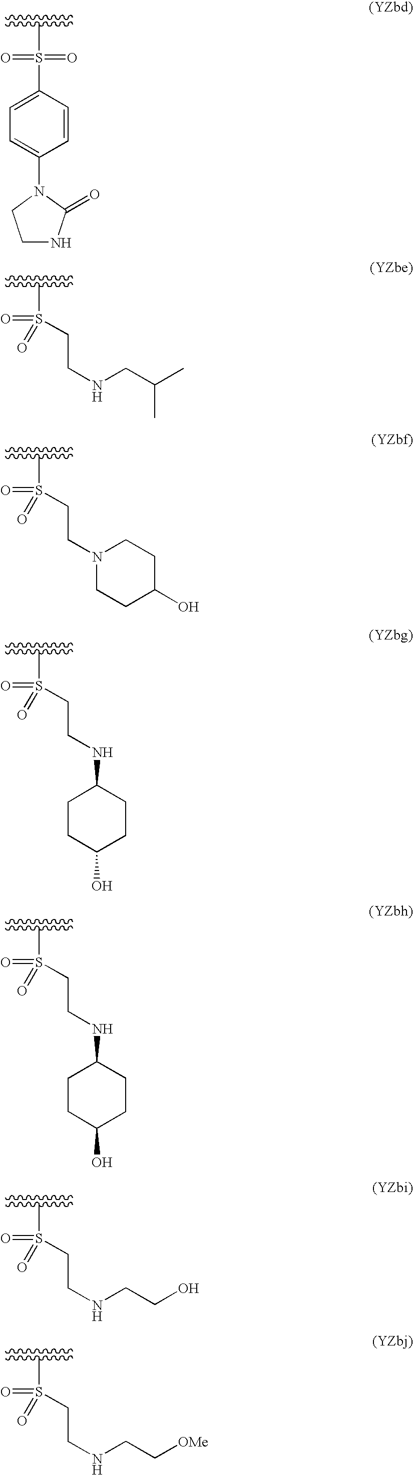

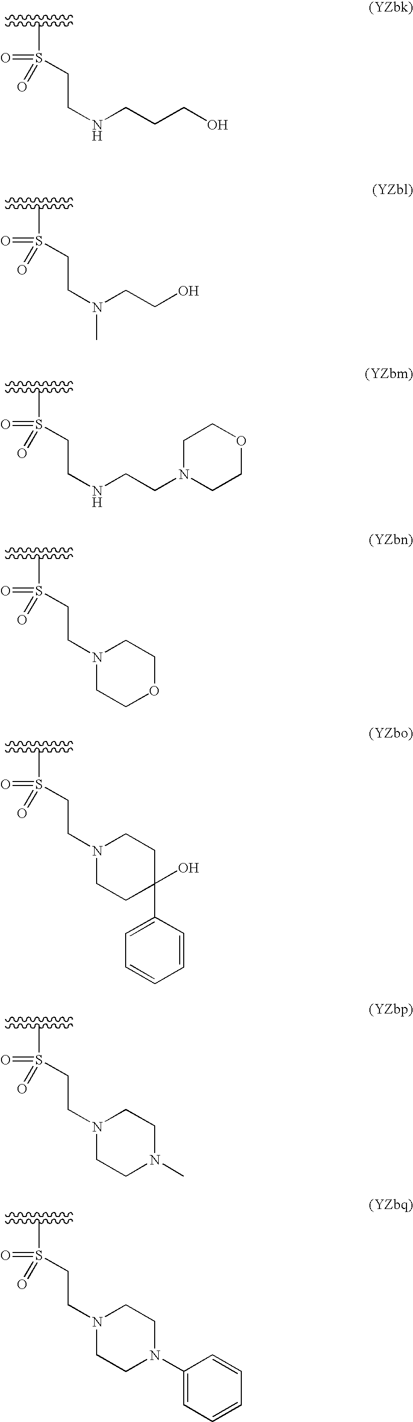

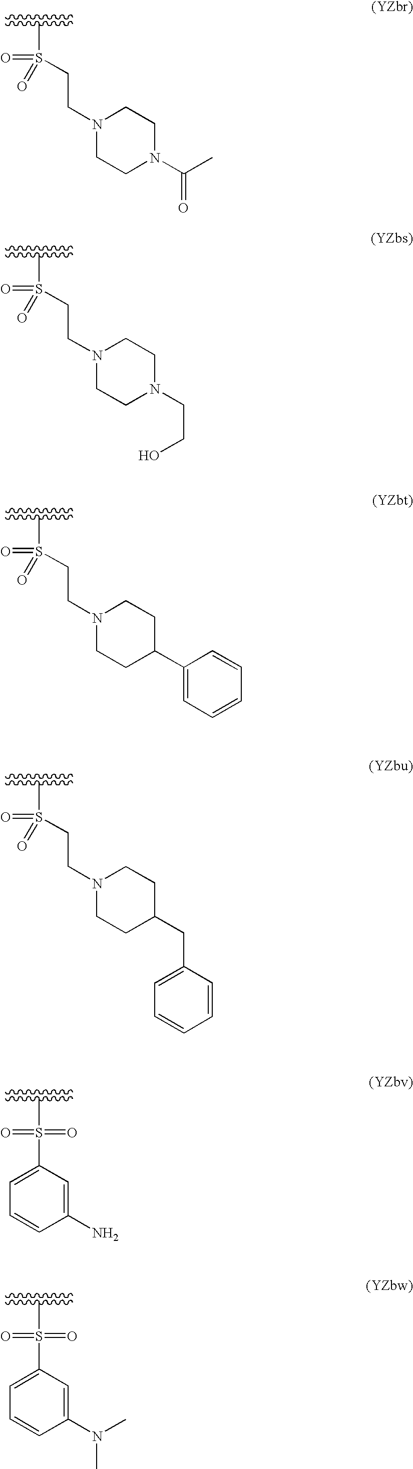

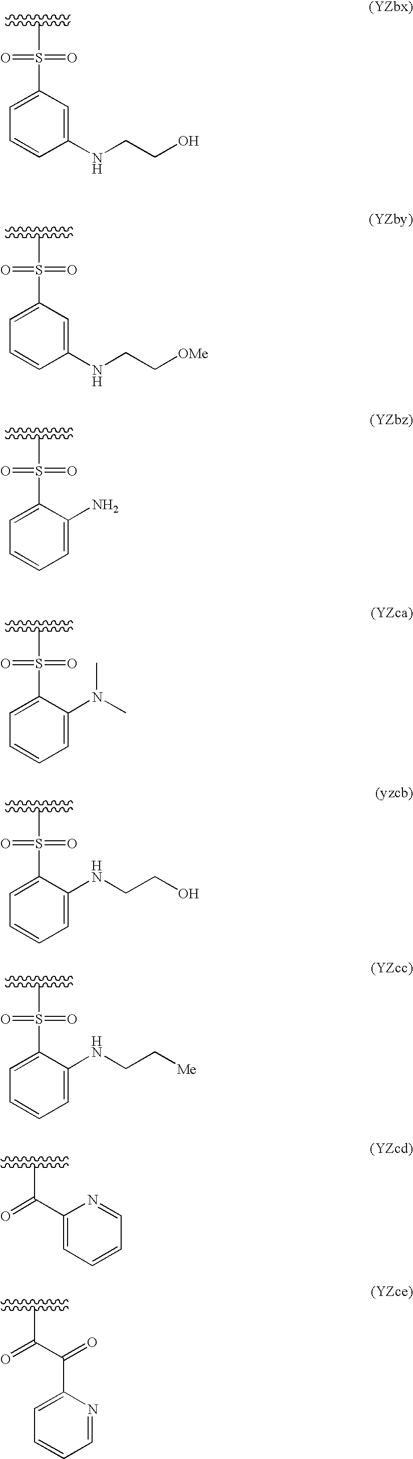

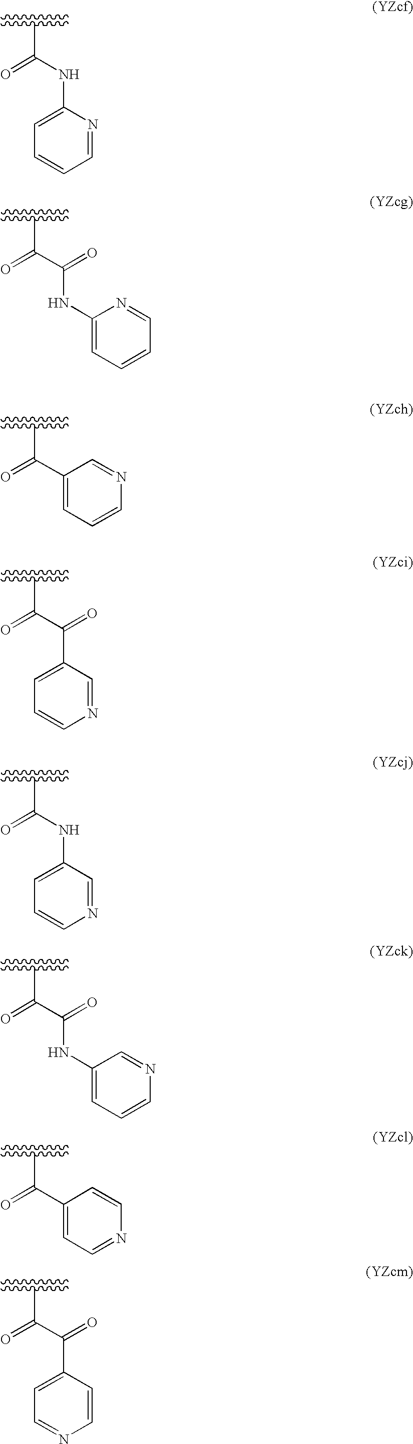

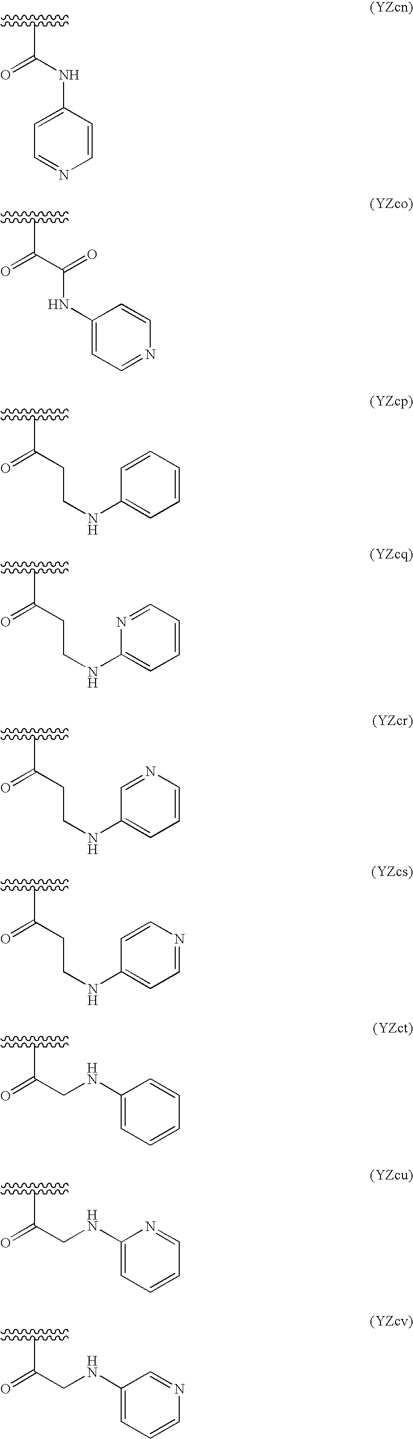

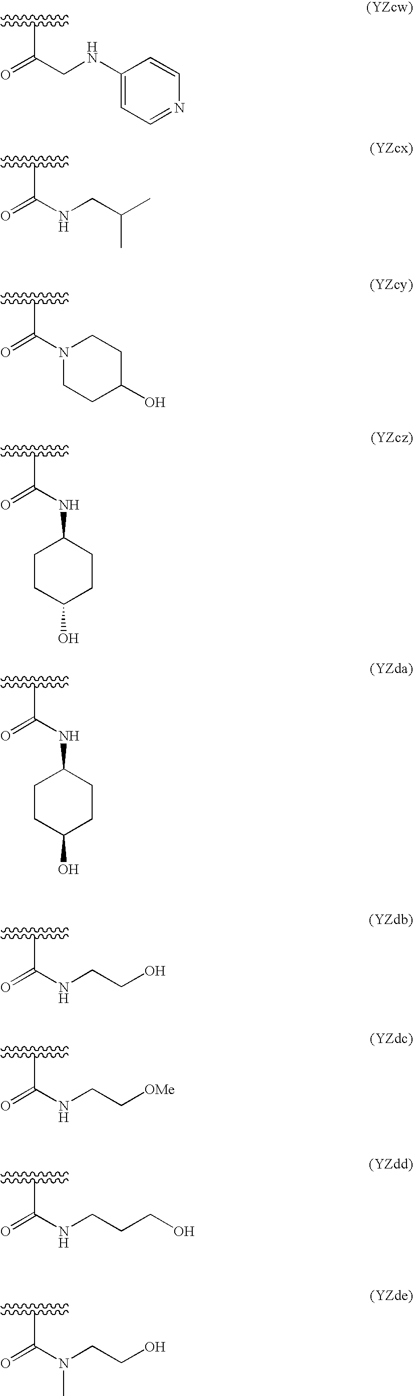

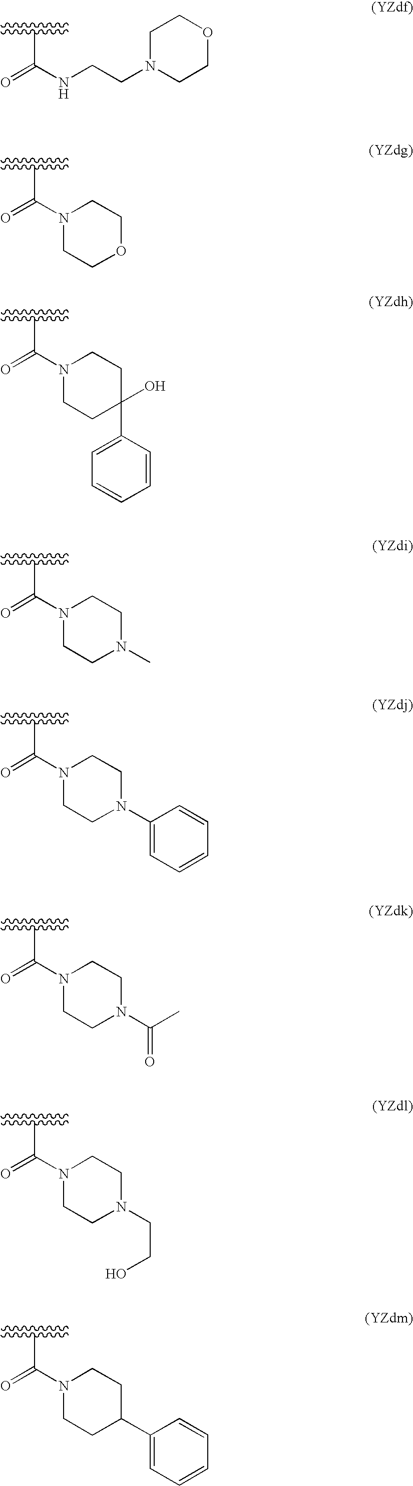

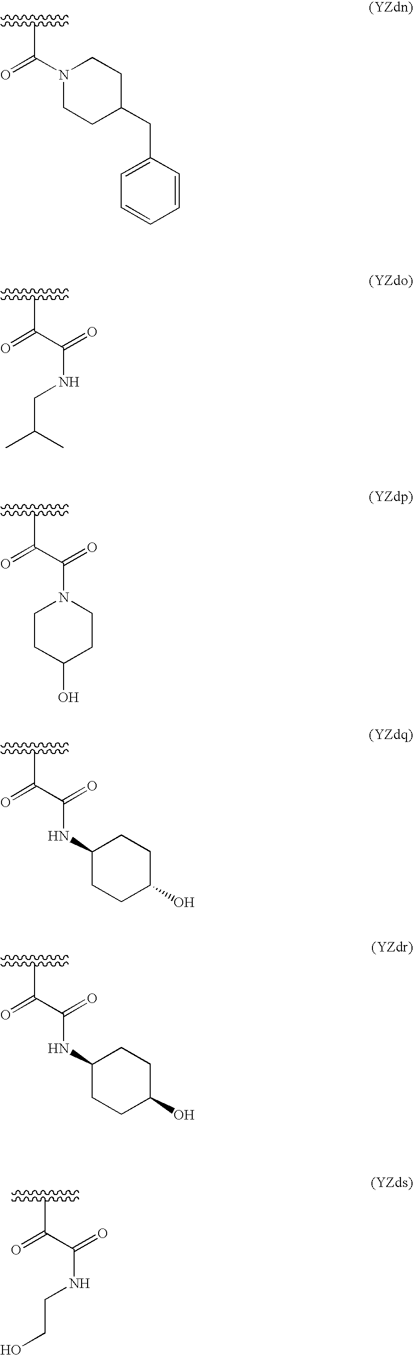

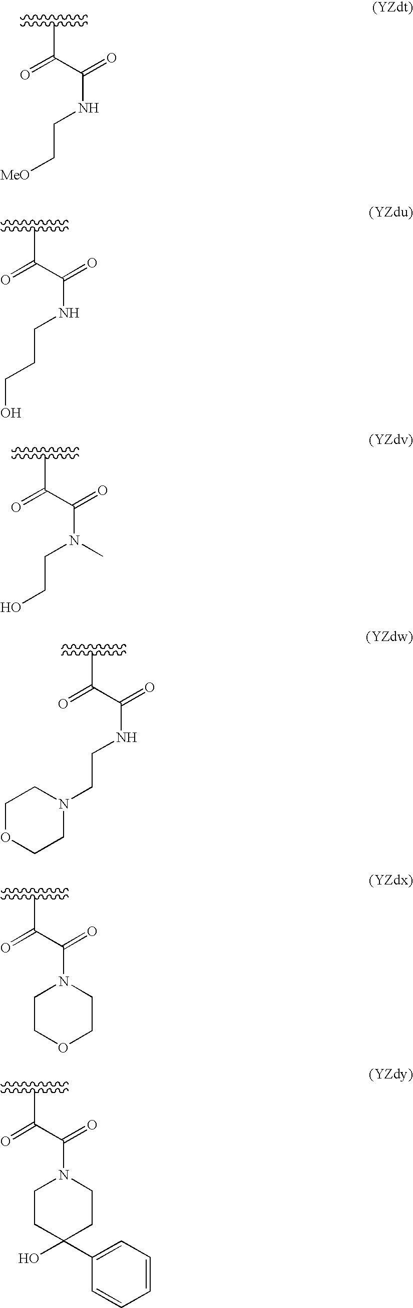

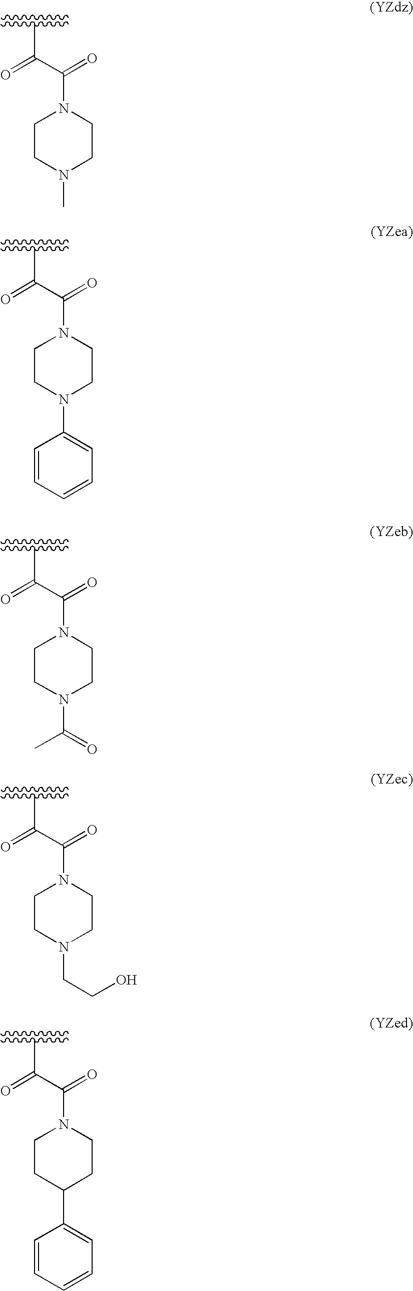

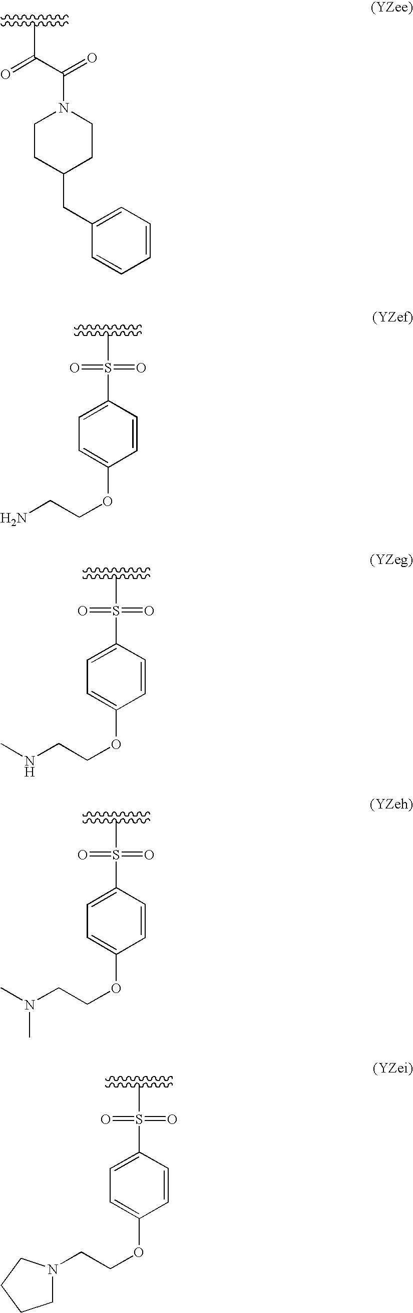

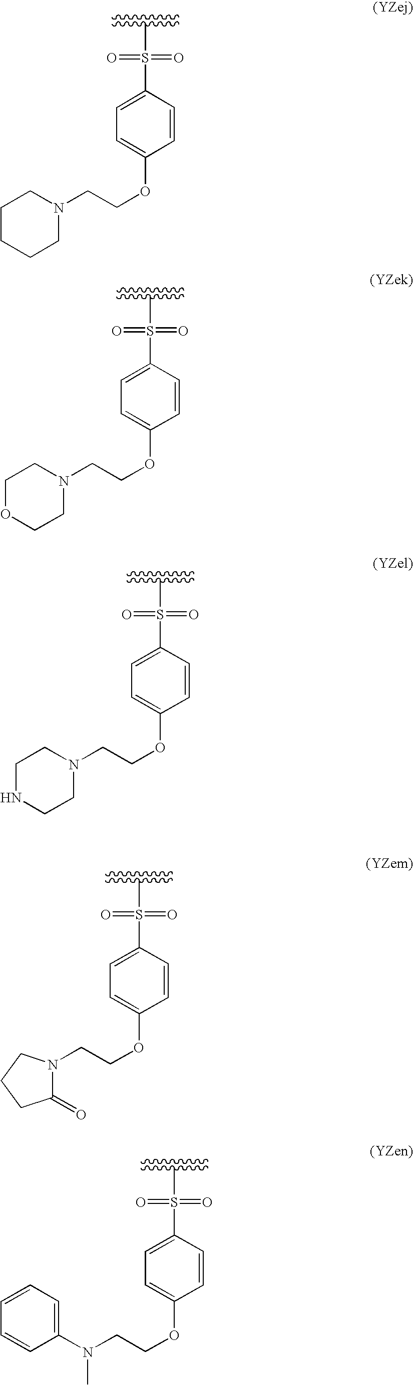

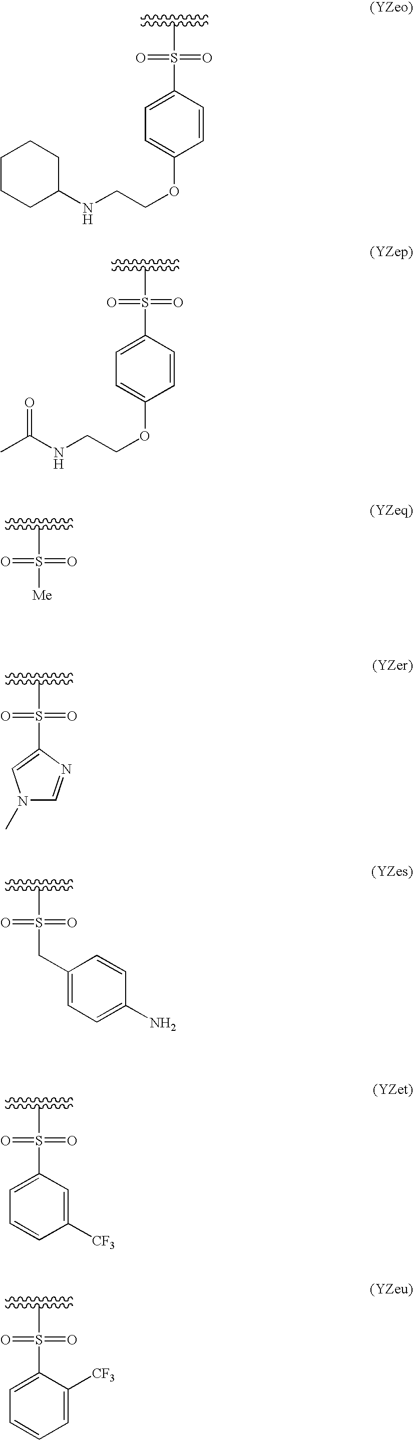

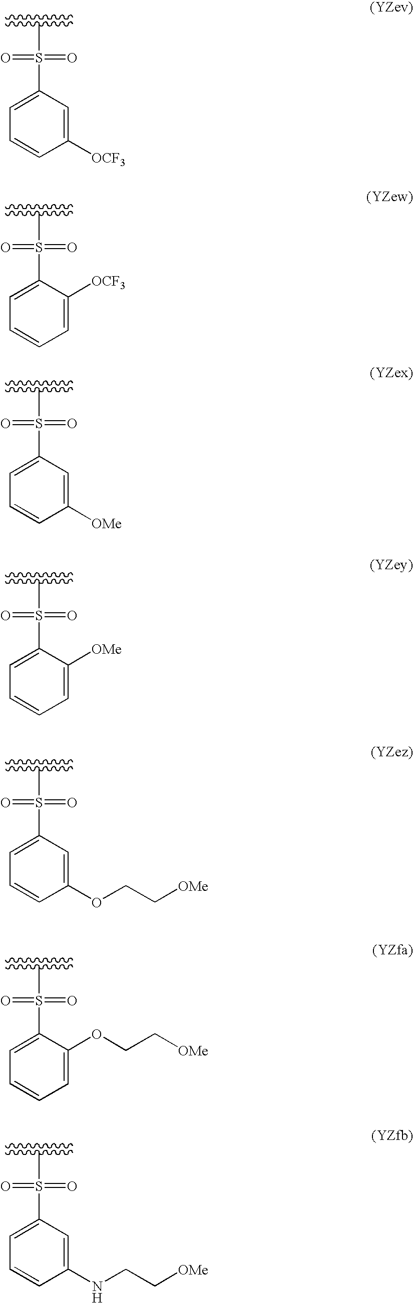

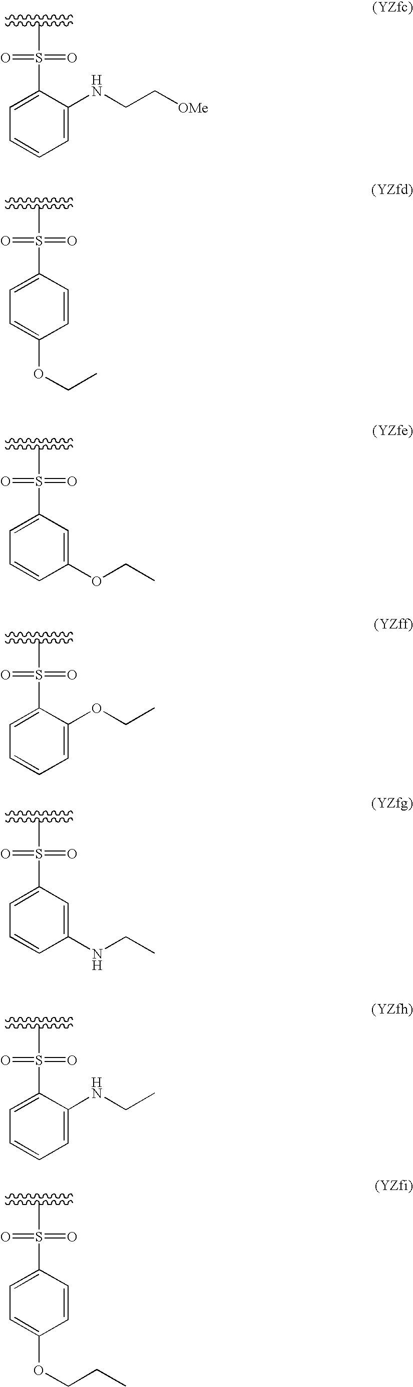

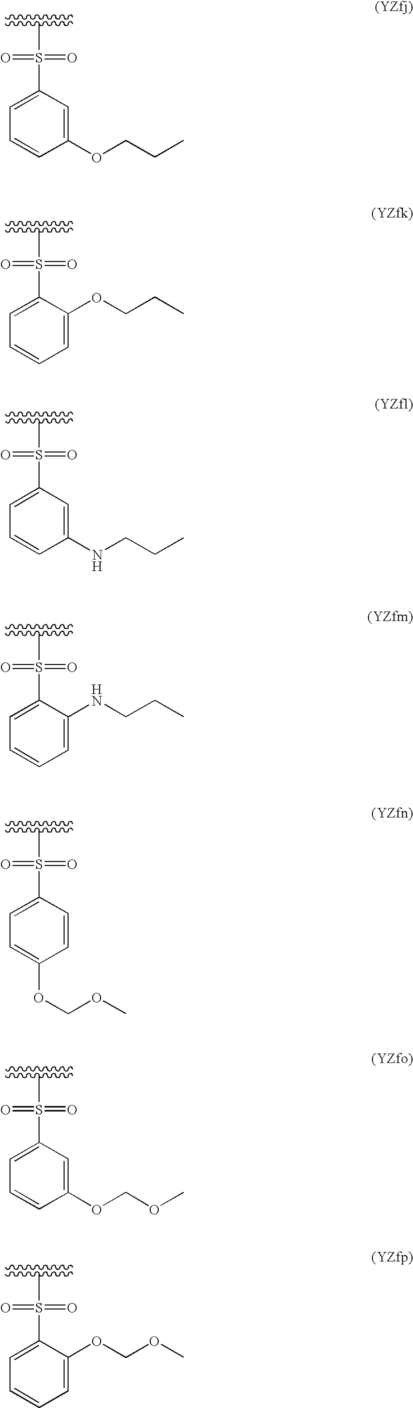

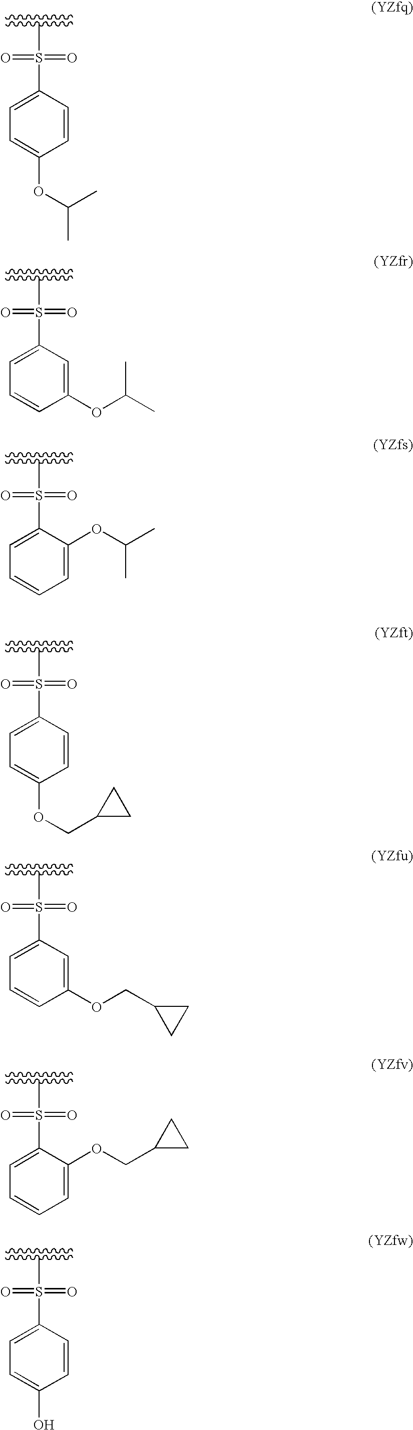

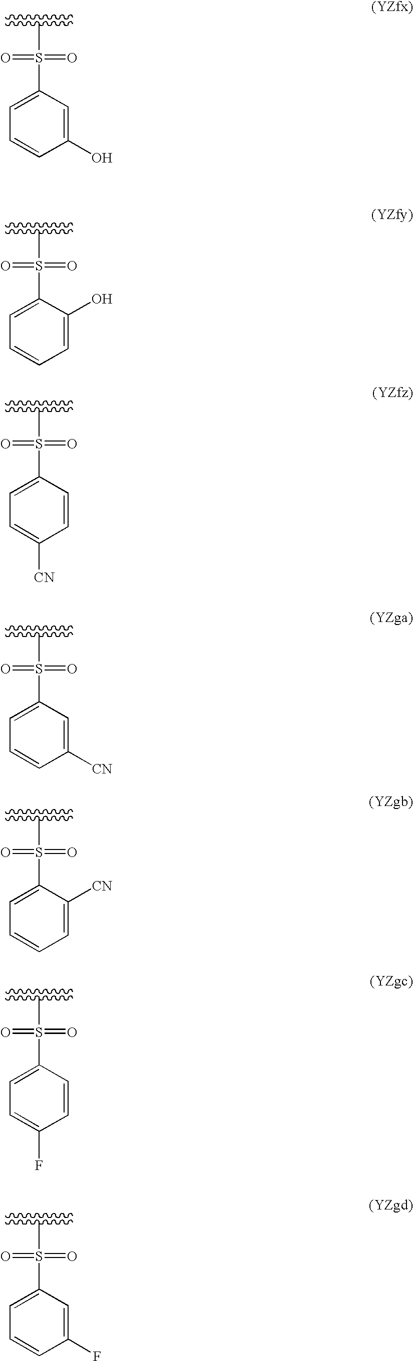

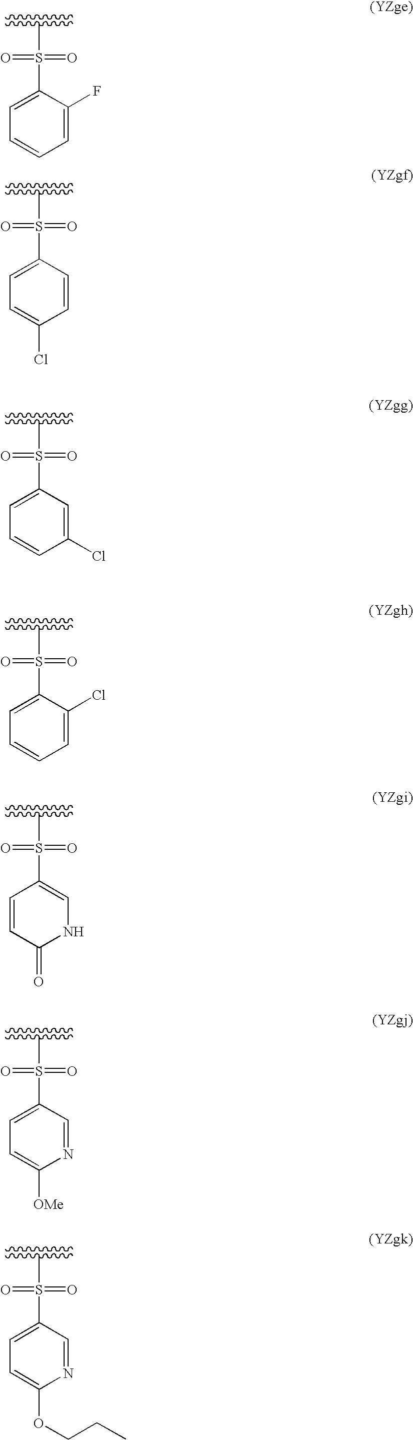

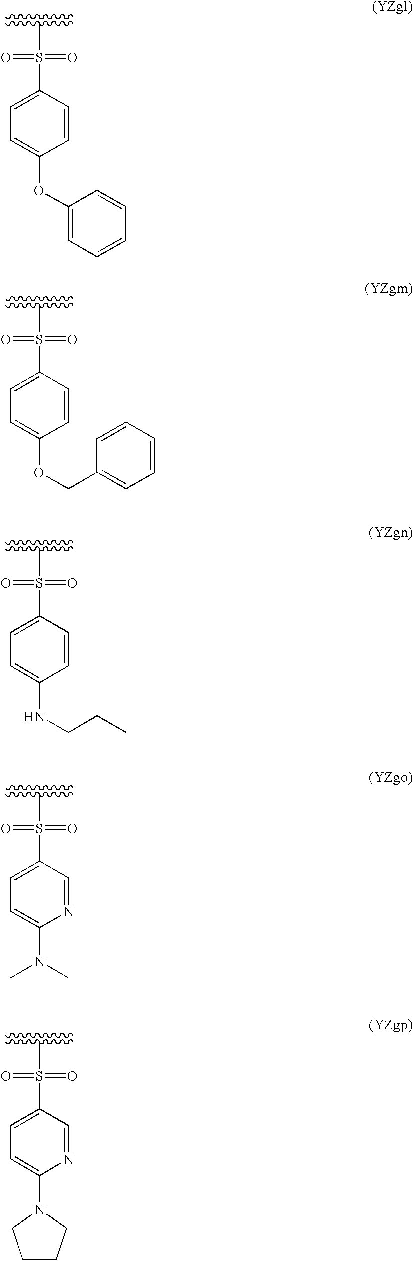

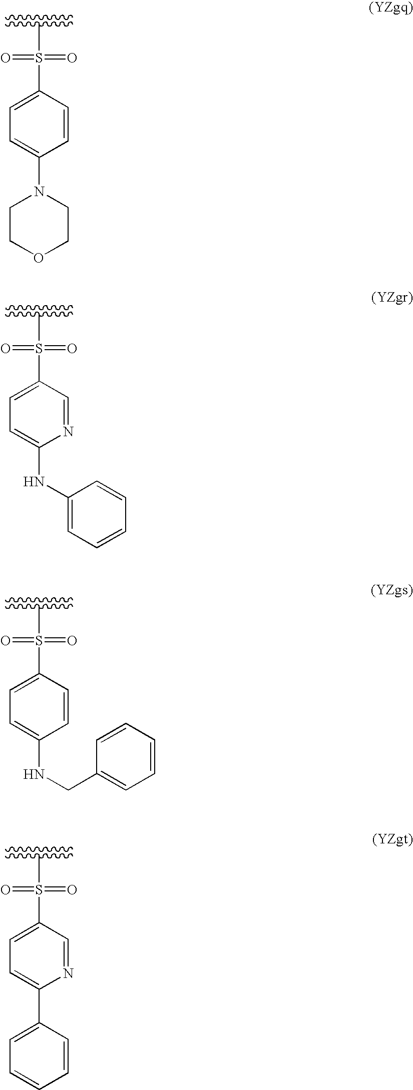

In another embodiment, the compounds I″ wherein the combination of X, CR¹R² and Y-Z (X, CR¹R², Y-Z) is one of the followings:

(X,CR¹R²,Y-Z)=(Xa,Ca,YZa),(Xa,Ca,YZb),(Xa,Ca,YZc),(Xa,Ca,YZd),(Xa,Ca,YZe),(Xa,Ca,YZf),(Xa, Ca,YZg),(Xa,Ca,YZh),(Xa,Ca,YZi),(Xa,Ca,YZj),(Xa,Ca,YZk),(Xa,Ca,YZl),(Xa,Ca,YZm), (Xa,Ca,YZn),(Xa,Ca,YZo),(Xa,Ca,YZp),(Xa,Ca,YZq),(Xa,Ca,YZr),(Xa,Ca,YZs), (Xa,Ca,YZt),(Xa,Ca,YZu),(Xa,Ca,YZv),(Xa,Ca,YZw),(Xa,Ca,YZx),(Xa,Ca,YZy),(Xa, Ca,YZz),(Xa,Ca,YZaa),(Xa,Ca,YZab),(Xa,Ca,YZac),(Xa,Ca,YZad),(Xa,Ca,YZae),(Xa, Ca,YZaf),(Xa,Ca,YZag),(Xa,Ca,YZah),(Xa,Ca,YZai),(Xa,Ca,YZaj),(Xa,Ca,YZak),(Xa, Ca,YZal),(Xa,Ca,YZam),(Xa,Ca,YZan),(Xa,Ca,YZao),(Xa,Ca,YZap),(Xa,Ca,YZaq), (Xa,Ca,YZar),(Xa,Ca,YZas),(Xa,Ca,YZat),(Xa,Ca,YZau),(Xa,Ca,YZav),(Xa,Ca,YZaw), (Xa,Ca,YZax),(Xa,Ca,YZay),(Xa,Ca,YZaz),(Xa,Ca,YZba),(Xa,Ca,YZbb),(Xa,Ca,YZbc), (Xa,Ca,YZbd),(Xa,Ca,YZbe),(Xa,Ca,YZbf),(Xa,Ca,YZbg),(Xa,Ca,YZbh),(Xa,Ca, YZbi),(Xa,Ca,YZbj),(Xa,Ca,YZbk),(Xa,Ca,YZbl),(Xa,Ca,YZbm),(Xa,Ca,YZbn),(Xa, Ca,YZbo),(Xa,Ca,YZbp),(Xa,Ca,YZbq),(Xa,Ca,YZbr),(Xa,Ca,YZbs),(Xa,Ca,YZbt),(Xa, Ca,YZbu),(Xa,Ca,YZbv),(Xa,Ca,YZbw),(Xa,Ca,YZbx),(Xa,Ca,YZby),(Xa,Ca,YZbz), (Xa,Ca,YZca),(Xa,Ca,YZcb),(Xa,Ca,YZcc),(Xa,Ca,YZcd),(Xa,Ca,YZce),(Xa,Ca,YZcf), (Xa,Ca,YZcg),(Xa,Ca,YZch),(Xa,Ca,YZci),(Xa,Ca,YZcj),(Xa,Ca,YZck),(Xa,Ca,YZcl), (Xa,Ca,YZcm),(Xa,Ca,YZcn),(Xa,Ca,YZco),(Xa,Ca,YZcp),(Xa,Ca,YZcq),(Xa,Ca, YZcr),(Xa,Ca,YZcs),(Xa,Ca,YZct),(Xa,Ca,YZcu),(Xa,Ca,YZcv),(Xa,Ca,YZcw),(Xa, Ca,YZcx),(Xa,Ca,YZcy),(Xa,Ca,YZcz),(Xa,Ca,YZda),(Xa,Ca,YZdb),(Xa,Ca,YZdc),(Xa, Ca,YZdd),(Xa,Ca,YZde),(Xa,Ca,YZdf),(Xa,Ca,YZdg),(Xa,Ca,YZdh),(Xa,Ca,YZdi), (Xa,Ca,YZdj),(Xa,Ca,YZdk),(Xa,Ca,YZdl),(Xa,Ca,YZdm),(Xa,Ca,YZdn),(Xa,Ca,YZdo), (Xa,Ca,YZdp),(Xa,Ca,YZdq),(Xa,Ca,YZdr),(Xa,Ca,YZds),(Xa,Ca,Yzdt),(Xa,Ca,YZdu), (Xa,Ca,YZdv),(Xa,Ca,YZdw),(Xa,Ca,YZdx),(Xa,Ca,YZdy),(Xa,Ca,YZdz),(Xa,Ca, YZea),(Xa,Ca,YZeb),(Xa,Ca,YZec),(Xa,Ca,YZed),(Xa,Ca,YZee),(Xa,Ca,YZef),(Xa, Ca,YZeg),(Xa,Ca,YZeh),(Xa,Ca,YZei),(Xa,Ca,YZej),(Xa,Ca,YZek),(Xa,Ca,YZel),(Xa, Ca,YZem),(Xa,Ca,YZen),(Xa,Ca,YZeo),(Xa,Ca,YZep),(Xa,Ca,YZeq),(Xa,Ca,YZer),(Xa, Ca,YZes),(Xa,Ca,YZet),(Xa,Ca,YZeu),(Xa,Ca,YZev),(Xa,Ca,YZew),(Xa,Ca,YZex), (Xa,Ca,YZey),(Xa,Ca,YZez),(Xa,Ca,YZfa),(Xa,Ca,YZfb),(Xa,Ca,YZfc),(Xa,Ca,YZfd), (Xa,Ca,YZfe),(Xa,Ca,YZff),(Xa,Ca,YZfg),(Xa,Ca,YZfh),(Xa,Ca,YZfi),(Xa,Ca,YZfj), (Xa,Ca,YZfk),(Xa,Ca,YZfl),(Xa,Ca,YZfm),(Xa,Ca,YZfn),(Xa,Ca,YZfo),(Xa,Ca,YZfp), (Xa,Ca,YZfq),(Xa,Ca,YZfr),(Xa,Ca,YZfs),(Xa,Ca,YZft),(Xa,Ca,YZfu),(Xa,Ca,YZfv), (Xa,Ca,YZfw),(Xa,Ca,YZfx),(Xa,Ca,YZfy),(Xa,Ca,YZfz),(Xa,Ca,YZga),(Xa,Ca,YZgb), (Xa,Ca,YZgc),(Xa,Ca,YZgd),(Xa,Ca,YZge),(Xa,Ca,YZgf),(Xa,Ca,YZgg),(Xa,Ca, YZgh),(Xa,Ca,YZgi),(Xa,Ca,YZgj),(Xa,Ca,YZgk),(Xa,Ca,YZgl),(Xa,Ca,YZgm),(Xa,Ca, YZgn),(Xa,Ca,YZgo),(Xa,Ca,YZgp),(Xa,Ca,YZgq),(Xa,Ca,YZgr),(Xa,Ca,YZgs),(Xa, Ca,YZgt),(Xa,Cb,YZa),(Xa,Cb,YZb),(Xa,Cb,YZc),(Xa,Cb,YZd),(Xa,Cb,YZe),(Xa, Cb,YZf),(Xa,Cb,YZg),(Xa,Cb,YZh),(Xa,Cb,YZi),(Xa,Cb,YZj),(Xa,Cb,YZk),(Xa,Cb, YZl),(Xa,Cb,YZm),(Xa,Cb,YZn),(Xa,Cb,YZo),(Xa,Cb,YZp),(Xa,Cb,YZq),(Xa,Cb,YZr), (Xa,Cb,YZs),(Xa,Cb,YZt),(Xa,Cb,YZu),(Xa,Cb,YZv),(Xa,Cb,YZw),(Xa,Cb,YZx),(Xa, Cb,YZy),(Xa,Cb,YZz),(Xa,Cb,YZaa),(Xa,Cb,YZab),(Xa,Cb,YZac),(Xa,Cb,YZad), (Xa,Cb,YZae),(Xa,Cb,YZaf),(Xa,Cb,YZag),(Xa,Cb,YZah),(Xa,Cb,YZai),(Xa,Cb,YZaj), (Xa,Cb,YZak),(Xa,Cb,YZal),(Xa,Cb,YZam);(Xa,Cb,YZan),(Xa,Cb,YZao),(Xa,Cb,YZap), (Xa,Cb,YZaq),(Xa,Cb,YZar),(Xa,Cb,YZas),(Xa,Cb,YZat),(Xa,Cb,YZau),(Xa,Cb, YZav),(Xa,Cb,YZaw),(Xa,Cb,YZax),(Xa,Cb,YZay),(Xa,Cb,YZaz),(Xa,Cb,YZba),(Xa, Cb,YZbb),(Xa,Cb,YZbc),(Xa,Cb,YZbd),(Xa,Cb,YZbe),(Xa,Cb,YZbf),(Xa,Cb,YZbg),(Xa, Cb,YZbh),(Xa,Cb,YZbi),(Xa,Cb,YZbj),(Xa,Cb,YZbk),(Xa,Cb,YZbl),(Xa,Cb,YZbm), (Xa,Cb,YZbn),(Xa,Cb,YZbo),(Xa,Cb,YZbp),(Xa,Cb,YZbq),(Xa,Cb,YZbr),(Xa,Cb, YZbs),(Xa,Cb,YZbt);(Xa,Cb,YZbu),(Xa,Cb,YZbv),(Xa,Cb,YZbw),(Xa,Cb,YZbx),(Xa, Cb,YZby),(Xa,Cb,YZbz),(Xa,Cb,YZca),(Xa,Cb,YZcb),(Xa,Cb,YZcc),(Xa,Cb,YZcd),(Xa, Cb,YZce),(Xa,Cb,YZcf),(Xa,Cb,YZcg),(Xa,Cb,YZch),(Xa,Cb,YZci),(Xa,Cb,YZcj), (Xa,Cb,YZck),(Xa,Cb,YZcl),(Xa,Cb,YZcm),(Xa,Cb,YZcn),(Xa,Cb,YZco),(Xa,Cb,YZcp), (Xa,Cb,YZcq),(Xa,Cb,YZcr),(Xa,Cb,YZcs),(Xa,Cb,YZct),(Xa,Cb,Ycu),(Xa,Cb, YZcv),(Xa,Cb,YZcw),(Xa,Cb,YZcx),(Xa,Cb,YZey),(Xa,Cb,YZcz),(Xa,Cb,YZda),(Xa, Cb,YZdb),(Xa,Cb,YZdc),(Xa,Cb,YZdd),(Xa,Cb,YZde),(Xa,Cb,YZdf),(Xa,Cb,YZdg),(Xa, Cb,YZdh),(Xa,Cb,YZdi),(Xa,Cb,YZdj),(Xa,Cb,YZdk),(Xa,Cb,YZdl),(Xa,Cb,YZdm), (Xa,Cb,YZdn),(Xa,Cb,YZdo),(Xa,Cb,YZ dp),(Xa,Cb,YZdq),(Xa,Cb,YZdr),(Xa,Cb, YZds),(Xa,Cb,Yzdt),(Xa,Cb,YZdu),(Xa,Cb,YZdv),(Xa,Cb,YZdw),(Xa,Cb,YZdx),(Xa, Cb,YZdy),(Xa,Cb,YZdz),(Xa,Cb,YZea),(Xa,Cb,YZeb),(Xa,Cb,YZec),(Xa,Cb,YZed),(Xa, Cb,YZee),(Xa,Cb,YZef),(Xa,Cb,YZeg),(Xa,Cb,YZeh),(Xa,Cb,YZei),(Xa,Cb,YZej), (Xa,Cb,YZek),(Xa,Cb,YZel),(Xa,Cb,YZem),(Xa,Cb,YZen),(Xa,Cb,YZeo),(Xa,Cb,YZep), (Xa,Cb,YZeq),(Xa,Cb,YZer),(Xa,Cb,YZes),(Xa,Cb,YZet),(Xa,Cb,YZeu),(Xa,Cb, YZev),(Xa,Cb,YZew),(Xa,Cb,YZex),(Xa,Cb,YZey),(Xa,Cb,YZez),(Xa,Cb,YZfa),(Xa, Cb,YZfb),(Xa,Cb,YZfc),(Xa,Cb,YZfd),(Xa,Cb,YZfe),(Xa,Cb,YZff),(Xa,Cb,YZfg),(Xa, Cb,YZfh),(Xa,Cb,YZfi),(Xa,Cb,YZfj),(Xa,Cb,YZfk),(Xa,Cb,YZfl),(Xa,Cb,YZfm),(Xa, Cb,YZfn),(Xa,Cb,YZfo),(Xa,Cb,YZfp),(Xa,Cb,YZfq),(Xa,Cb,YZfr),(Xa,Cb,YZfs),(Xa, Cb,YZft),(Xa,Cb,YZfu),(Xa,Cb,YZfv),(Xa,Cb,YZfw),(Xa,Cb,YZfx),(Xa,Cb,YZfy), (Xa,Cb,YZfz),(Xa,Cb,YZga),(Xa,Cb,YZgb),(Xa,Cb,YZgc),(Xa,Cb,YZgd),(Xa,Cb,YZge), (Xa,Cb,YZgf),(Xa,Cb,YZgg),(Xa,Cb,YZgh),(Xa,Cb,YZgi),(Xa,Cb,YZgj),(Xa,Cb, YZgk),(Xa,Cb,YZgl),(Xa,Cb,YZgm),(Xa,Cb,YZgn),(Xa,Cb,YZgo),(Xa,Cb,YZgp),(Xa, Cb,YZgq),(Xa,Cb,YZgr),(Xa,Cb,YZgs),(Xa,Cb,YZgt),(Xa,Cc,YZa),(Xa,Cc,YZb),(Xa, Cc,YZc),(Xa,Cc,YZd),(Xa,Cc,YZe),(Xa,Cc,YZf),(Xa,Cc,YZg),(Xa,Cc,YZh),(Xa,Cc, YZi),(Xa,Cc,YZj),(Xa,Cc,YZk),(Xa,Cc,YZl),(Xa,Cc,YZm),(Xa,Cc,YZn),(Xa,Cc,YZo), (Xa,Cc,YZp),(Xa,Cc,YZq),(Xa,Cr,YZr),(Xa,Cc,YZs),(Xa,Cc,YZt),(Xa,Cc,YZu),(Xa, Cc,YZv),(Xa,Cc,YZw),(Xa,Cc,YZx),(Xa,Cc,YZy),(Xa,Cc,YZz),(Xa,Cc,YZaa),(Xa,Cc, YZab),(Xa,Cc,YZac),(Xa,Cc,YZad),(Xa,Cc,YZae),(Xa,Cc,YZaf),(Xa,Cc,YZag),(Xa, Cc,YZah),(Xa,Cc,YZai),(Xa,Cc,YZaj),(Xa,Cc,YZak),(Xa,Cc,YZal),(Xa,Cc,YZam),(Xa, Cc,YZan),(Xa,Cc,YZao),(Xa,Cc,YZap),(Xa,Cc,YZaq),(Xa,Cc,YZar),(Xa,Cc,YZas),(Xa, Cc,YZat),(Xa,Cc,YZau),(Xa,Cc,YZav),(Xa,Cc,YZaw),(Xa,Cc,YZax),(Xa,Cc,YZay), (Xa,Cc,YZaz),(Xa,Cc,YZba),(Xa,Cc,YZbb),(Xa,Cc,YZbc),(Xa,Cc,YZbd),(Xa,Cc,YZbe), (Xa,Cc,YZbf),(Xa,Cc,YZbg),(Xa,Cc,YZbh),(Xa,Cc,YZbi),(Xa,Cc,YZbj),(Xa,Cc,YZbk), (Xa,Cc,YZbl),(Xa,Cc,YZbm),(Xa,Cc,YZbn),(Xa,Cc,YZbo),(Xa,Cc,YZbp),(Xa,Cc, YZbq),(Xa,Cc,YZbr),(Xa,Cc,YZbs),(Xa,Cc,YZbt),(Xa,Cc,YZbu);(Xa,Cc,YZbv),(Xa, Cc,YZbw),(Xa,Cc,YZbx),(Xa,Cc,YZby),(Xa,Cc,YZbz),(Xa,Cc,YZca),(Xa,Cc,YZcb),(Xa, Cc,YZcc),(Xa,Cc,YZcd),(Xa,Cc,YZce),(Xa,Cc,YZcf),(Xa,Cc,YZcg),(Xa,Cc,YZch), (Xa,Cc,YZci),(Xa,Cc,YZcj),(Xa,Cc,YZck),(Xa,Cc,YZcl),(Xa,Cc,YZcm),(Xa,Cc,YZcn), (Xa,Cc,YZco),(Xa,Cc,YZcp),(Xa,Cc,YZcq),(Xa,Cc,YZcr),(Xa,Cc,YZcs),(Xa,Cc,YZt), (Xa,Cc,YZeu),(Xa,Cc,YZcv),(Xa,Cc,YZcw),(Xa,Cc,YZcx),(Xa,Cc,YZcy),(Xa,Cc, YZcz),(Xa,Cc,YZda),(Xa,Cc,YZdb),(Xa,Cc,YZdc),(Xa,Cc,YZdd),(Xa,Cc,YZde),(Xa, Cc,YZdf),(Xa,Cc,YZdg),(Xa,Cc,YZdh),(Xa,Cc,YZdi),(Xa,Cc,YZdj),(Xa,Cc,YZdk),(Xa, Cc,YZdl),(Xa,Cc,YZdm),(Xa,Cc,YZdn),(Xa,Cc,YZdo),(Xa,Cc,YZdp),(Xa,Cc,YZdq), (Xa,Cc,YZdr),(Xa,Cc,YZds),(Xa,Cc,Yzdt),(Xa,Cc,YZdu),(Xa,Cc,YZdv),(Xa,Cc,YZdw), (Xa,Cc,YZdx),(Xa,Cc,YZdy),(Xa,Cc,YZdz),(Xa,Cc,YZea),(Xa,Cc,YZeb),(Xa,Cc, YZec),(Xa,Cc,YZed),(Xa,Cc,YZee),(Xa,Cc,YZef),(Xa,Cc,YZeg),(Xa,Cc,YZeh),(Xa,Cc, YZei),(Xa,Cc,YZej),(Xa,Cc,YZek),(Xa,Cc,YZel),(Xa,Cc,YZem),(Xa,Cc,YZen),(Xa, Cc,YZeo),(Xa,Cc,YZep),(Xa,Cc,YZeq),(Xa,Cc,YZer),(Xa,Cc,YZes),(Xa,Cc,YZet),(Xa, Cc,YZeu),(Xa,Cc,YZev),(Xa,Cc,YZew),(Xa,Cc,YZex),(Xa,Cc,YZey),(Xa,Cc,YZez), (Xa,Cc,YZfa),(Xa,Cc,YZfb),(Xa,Cc,YZfc),(Xa,Cc,YZfd),(Xa,Cc,YZfe),(Xa,Cc,YZff), (Xa,Cc,YZfg),(Xa,Cc,YZfh),(Xa,Cc,YZfi),(Xa,Cc,YZfj),(Xa,Cc,YZfk),(Xa,Cc,YZfl),(Xa, Cc,YZfm),(Xa,Cc,YZfn),(Xa,Cc,YZfo),(Xa,Cc,YZfp,(Xa,Cc,YZfq),(Xa,Cc,YZfr), (Xa,Cc,YZfs),(Xa,Cc,YZft),(Xa,Cc,YZfu),(Xa,Cc,YZfv),(Xa,Cc,YZfwv),(Xa,Cc,YZfx), (Xa,Cc,YZfy),(Xa,Cc,YZfz),(Xa,Cc,YZga),(Xa,Cc,YZgb),(Xa,Cc,YZge),(Xa,Cc,YZgd), (Xa,Cc,YZge),(Xa,Cc,YZgf),(Xa,Cc,YZgg),(Xa,Cc,YZgh),(Xa,Cc,YZgi),(Xa,Cc,YZgj), (Xa,Cc,YZgk),(Xa,Cc,YZgl),(Xa,Cc,YZgm),(Xa,Cc,YZgn),(Xa,Cc,YZgo),(Xa,Cc, YZgp),(Xa,Cc,YZgq),(Xa,Cc,YZgr),(Xa,Cc,YZgs),(Xa,Cc,YZgt),(Xa,Cd,YZa),(Xa, Cd,YZb),(Xa,Cd,YZc),(Xa,Cd,YZd),(Xa,Cd,YZe),(Xa,Cd,YZf),(Xa,Cd,YZg),(Xa,Cd, YZh),(Xa,Cd,YZi),(Xa,Cd,YZj),(Xa,Cd,YZk),(Xa,Cd,YZl),(Xa,Cd,YZm),(Xa,Cd,YZn), (Xa,Cd,YZo),(Xa,Cd,YZp),(Xa,Cd,YZq),(Xa,Cd,YZr),(Xa,Cd,YZs),(Xa,Cd,YZt),(Xa, Cd,YZu),(Xa,Cd,YZv),(Xa,Cd,YZw),(Xa,Cd,YZx),(Xa,Cd,YZy),(Xa,Cd,YZz),(Xa, Cd,YZaa),(Xa,Cd,YZab),(Xa,Cd,YZac),(Xa,Cd,YZad),(Xa,Cd,YZae),(Xa,Cd,YZaf),(Xa, Cd,YZag),(Xa,Cd,YZah),(Xa,Cd,YZai),(Xa,Cd,YZaj),(Xa,Cd,YZak),(Xa,Cd,YZal), (Xa,Cd,YZam),(Xa,Cd,YZan),(Xa,Cd,YZao),(Xa,Cd,YZap),(Xa,Cd,YZaq),(Xa,Cd,YZar), (Xa,Cd,YZas),(Xa,Cd,YZat),(Xa,Cd,YZau),(Xa,Cd,YZav),(Xa,Cd,YZaw),(Xa,Cd, YZax),(Xa,Cd,YZay),(Xa,Cd,YZaz),(Xa,Cd,YZba),(Xa,Cd,YZbb),(Xa,Cd,YZbc),(Xa, Cd,YZbd),(Xa,Cd,YZbe),(Xa,Cd,YZbf),(Xa,Cd,YZbg),(Xa,Cd,YZbh),(Xa,Cd,YZbi),(Xa, Cd,YZbj),(Xa,Cd,YZbk),(Xa,Cd,YZbl),(Xa,Cd,YZbm),(Xa,Cd,YZbn),(Xa,Cd,YZbo), (Xa,Cd,YZbp),(Xa,Cd,YZbq),(Xa,Cd,YZbr),(Xa,Cd,YZbs),(Xa,Cd,YZbt),(Xa,Cd,YZbu), (Xa,Cd,YZbv),(Xa,Cd,YZbw),(Xa,Cd,YZbx),(Xa,Cd,YZby),(Xa,Cd,YZbz),(Xa, Cd,YZca),(Xa,Cd,YZcb),(Xa,Cd,YZcc),(Xa,Cd,YZcd),(Xa,Cd,YZce),(Xa,Cd,YZef),(Xa, Cd,YZcg),(Xa,Cd,YZch),(Xa,Cd,YZci),(Xa,Cd,Yscj),(Xa,Cd,YZck),(Xa,Cd,YZcl), (Xa,Cd,YZcm),(Xa,Cd,YZcn),(Xa,Cd,YZco),(Xa,Cd,YZcp),(Xa,Cd,YZcq),(Xa,Cd,YZcr), (Xa,Cd,YZcs),(Xa,Cd,YZct),(Xa,Cd,YZcu),(Xa,Cd,YZcv),(Xa,Cd,YZcw),(Xa,Cd, YZcx),(Xa,Cd,YZcy),(Xa,Cd,YZcz),(Xa,Cd,YZda),(Xa,Cd,YZdb),(Xa,Cd,YZdc),(Xa, Cd,YZdd),(Xa,Cd,YZde),(Xa,Cd,YZdf),(Xa,Cd,YZdg),(Xa,Cd,YZdh),(Xa,Cd,YZdi),(Xa, Cd,YZdj),(Xa,Cd,YZdk),(Xa,Cd,YZdl),(Xa,Cd,YZdm),(Xa,Cd,YZdn),(Xa,Cd,YZdo), (Xa,Cd,YZdp),(Xa,Cd,YZdq),(Xa,Cd,YZdr),(Xa,Cd,YZds),(Xa,Cd,Yzdt),(Xa,Cd,YZdu), (Xa,Cd,YZdv),(Xa,Cd,YZdw),(Xa,Cd,YZdx),(Xa,Cd,YZdy),(Xa,Cd,YZdz),(Xa, Cd,YZea),(Xa,Cd,YZeb),(Xa,Cd,YZec),(Xa,Cd,YZed),(Xa,Cd,YZee),(Xa,Cd,YZef),(Xa, Cd,YZeg),(Xa,Cd,YZeh),(Xa,Cd,YZei),(Xa,Cd,YZej),(Xa,Cd,YZek),(Xa,Cd,YZel), (Xa,Cd,YZem),(Xa,Cd,YZen),(Xa,Cd,YZeo),(Xa,Cd,YZep),(Xa,Cd,YZeq),(Xa,Cd,YZer), (Xa,Cd,YZes),(Xa,Cd,YZet),(Xa,Cd,YZeu),(Xa,Cd,YZev),(Xa,Cd,YZew),(Xa,Cd, YZex),(Xa,Cd,YZey),(Xa,Cd,YZez),(Xa,Cd,YZfa),(Xa,Cd,YZfb),(Xa,Cd,YZfc),(Xa, Cd,YZfd),(Xa,Cd,YZfe),(Xa,Cd,YZff),(Xa,Cd,YZfg),(Xa,Cd,YZfh),(Xa,Cd,YZfi),(Xa, Cd,YZfj),(Xa,Cd,YZfk),(Xa,Cd,YZfl),(Xa,Cd,YZfm),(Xa,Cd,YZfn),(Xa,Cd,YZfo),(Xa, Cd,YZfp),(Xa,Cd,YZfq),(Xa,Cd,YZfr),(Xa,Cd,YZfs),Xxa,Cd,YZft),(Xa,Cd,YZfu),(Xa, Cd,YZfv),(Xa,Cd,YZfw),(Xa,Cd,YZfx),(Xa,Cd,YZfy),(Xa,Cd,YZfz),(Xa,Cd,YZga), (Xa,Cd,YZgb),(Xa,Cd,YZgc),(Xa,Cd,YZgd),(Xa,Cd,YZge,),(Xa,Cd,YZgf),(Xa,Cd,YZgg), (Xa,Cd,YZgh),(Xa,Cd,YZgi),(Xa,Cd,YZgj),(Xa,Cd,YZgk),(Xa,Cd,YZgl),(Xa,Cd, YZgm),(Xa,Cd,YZgn),(Xa,Cd,YZgo),(Xa,Cd,YZgp),(Xa,Cd,YZgq),(Xa,Cd,YZgr),(Xa, Cd,YZgs),(Xa,Cd,YZgt),(Xa,Ce,YZa),(Xa,Ce,YZb),(Xa,Ce,YZc),(Xa,Ce,YZd),(Xa,Ce, YZe),(Xa,Ce,YZf),(Xa,Ce,YZg),(Xa,Ce,YZh),(Xa,Ce,YZi),(Xa,Ce,YZj),(Xa,Ce,YZk), (Xa,Ce,YZl),(Xa,Ce,YZm),(Xa,Ce,YZn),(Xa,Ce,YZo),(Xa,Ce,YZp),(Xa,Ce,YZq),(Xa, Ce,YZr),(Xa,Ce,YZs),(Xa,Ce,YZt),(Xa,Ce,YZu),(Xa,Ce,YZv),(Xa,Ce,YZw),(Xa,Ce, YZx),(Xa,Ce,YZy),(Xa,Ce,YZz),(Xa,Ce,YZaa),(Xa,Ce,YZab),(Xa,Ce,YZac),(Xa,Ce, YZad),(Xa,Ce,YZae),(Xa,Ce,YZaf),(Xa,Ce,YZag),(Xa,Ce,YZah),(Xa,Ce,YZai),(Xa,Ce, YZaj),(Xa,Ce,YZak),(Xa,Ce,YZal),(Xa,Ce,YZam),(Xa,Ce,YZan),(Xa,Ce,YZao),(Xa, Ce,YZap),(Xa,Ce,YZaq),(Xa,Ce,YZar),(Xa,Ce,YZas),(Xa,Ce,YZat),(Xa,Ce,YZau),(Xa, Ce,YZav),(Xa,Ce,YZaw),(Xa,Ce,YZax),(Xa,Ce,YZay),(Xa,Ce,YZaz),(Xa,Ce,YZba), (Xa,Ce,YZbb),(Xa,Ce,YZbc),(Xa,Ce,YZbd),(Xa,Ce,YZbe),(Xa,Ce,YZbf),(Xa,Ce,YZbg), (Xa,Ce,YZbh),(Xa,Ce,YZbi),(Xa,Ce,YZbj),(Xa,Ce,YZbk),(Xa,Ce,YZbl),(Xa,Ce,YZbm), (Xa,Ce,YZbn),(Xa,Ce,YZbo),(Xa,Ce,YZbp),(Xa,Ce,YZbq),(Xa,Ce,YZbr),(Xa,Ce, YZbs),(Xa,Ce,YZbt),(Xa,Ce,YZbu),(Xa,Ce,YZbv),(Xa,Ce,YZbw),(Xa,Ce,YZbx),(Xa, Ce,YZby),(Xa,Ce,YZbz),(Xa,Ce,YZca),(Xa,Ce,YZcb),(Xa,Ce,YZcc),(Xa,Ce,YZed),(Xa, Ce,YZce),(Xa,Ce,YZcf),(Xa,Ce,YZcg),(Xa,Ce,YZch),(Xa,Ce,YZci),(Xa,Ce,YZcj), (Xa,Ce,YZck),(Xa,Ce,YZcl),(Xa,Ce,YZcm),(Xa,Ce,YZcn),(Xa,Ce,YZco),(Xa,Ce,YZcp), (Xa,Ce,YZcq),(Xa,Ce,YZcr),(Xa,Ce,YZcs),(Xa,Ce,YZct),(XaiCe,YZcu),(Xa,Ce,YZev), (Xa,Ce,YZcw),(Xa,Ce,YZcx),(Xa,Ce,YZcy),(Xa,Ce,YZcz),(Xa,Ce,YZda),(Xa,Ce, YZdb),(Xa,Ce,YZdc),(Xa,Ce,YZdd),(Xa,Ce,YZde),(Xa,Ce,YZdf),(Xa,Ce,YZdg),(Xa, Ce,YZdh),(Xa,Ce,YZdi),(Xa,Ce,YZdj),(Xa,Ce,YZdk),(Xa,Ce,YZdl),(Xa,Ce,YZdm),(Xa, Ce,YZdn),(Xa,Ce,YZdo),(Xa,Ce,YZdp),(Xa,Ce,YZdq),(Xa,Ce,YZdr),(Xa,Ce,YZds), (Xa,Ce,Yzdt),(Xa,Ce,YZdu),(Xa,Ce,YZdv),(Xa,Ce,YZdw),(Xa,Ce,YZdx),(Xa,Ce,YZdy), (Xa,Ce,YZdz),(Xa,Ce,YZea),(Xa,Ce,YZeb),(Xa,Ce,YZec),(Xa,Ce,YZed),(Xa,Ce, YZee),(Xa,Ce,YZef),(Xa,Ce,YZeg),(Xa,Ce,YZeh),(Xa,Ce,YZei),(Xa,Ce,YZej),(Xa,Ce, YZek),(Xa,Ce,YZel),(Xa,Ce,YZem),(Xa,Ce,YZen),(Xa,Ce,YZeo),(Xa,Ce,YZep),(Xa, Ce,YZeq),(Xa,Ce,YZer),(Xa,Ce,YZes),(Xa,Ce,YZet),(Xa,Ce,YZeu),(Xa,Ce,YZev),(Xa, Ce,YZew),(Xa,Ce,YZex),(Xa,Ce,YZey),(Xa,Ce,YZez),(Xa,Ce,YZfa),(Xa,Ce,YZfb),(Xa, Ce,YZfc),(Xa,Ce,YZfd),(Xa,Ce,YZfe),(Xa,Ce,YZff),(Xa,Ce,YZfg),(Xa,Ce,YZfh),(Xa, Ce,YZfi),(Xa,Ce,YZfj),(Xa,Ce,YZfk),(Xa,Ce,YZfl),(Xa,Ce,YZfm),(Xa,Ce,YZfn),(Xa, Ce,YZfo),(Xa,Ce,YZfp),(Xa,Ce,YZfq),(Xa,Ce,YZfr),(Xa,Ce,YZfs),(Xa,Ce,YZft),(Xa, Ce,Yfiu),(Xa, Ce,YZfv),(Xa,Ce,YZfw),(Xa,Ce,YZfx),(Xa,Ce,YZfy),(Xa,Ce,YZfz), (Xa,Ce,YZga),(Xa,Ce,YZgb),(Xa,Ce,YZgc),(Xa,Ce,YZgd),(Xa,Ce,yZge),(Xa,Ce,YZgf), (Xa,Ce,YZgg),(Xa,Ce,YZgh),(Xa,Ce,YZgi),(Xa,Ce,YZzj),(Xa,Ce,YZgk),(Xa,Ce,YZgl), (Xa,Ce,YZgm),(Xa,Ce,YZgn),(Xa,Ce,YZgo),(Xa,Ce,YZgp),(Xa,Ce,YZgq),(Xa,Ce, YZgr),(Xa,Ce,YZgs),(Xa,Ce,YZgt),(Xa,Cf,YZa),(Xa,Cf,YZb),(Xa,Cf,YZc),(Xa,Cf, YZd),(Xa,Cf,YZe),(Xa,Cf,YZf),(Xa,Cf,YZg),(Xa,Cf,YZh),(Xa,Cf,YZi),(Xa,Cf,YZj),(Xa, Cf,YZk),(Xa,Cf,YZl),(Xa,Cf,YZm),(Xa,Cf,YZn),(Xa,Cf,YZo),(Xa,Cf,YZp),(Xa,Cf, YZq),(Xa,Cf,YZr),(Xa,Cf,YZs),(Xa,Cf,YZt),(Xa,Cf,YZu),(Xa,Cf,YZv),(Xa,Cf,YZw), (Xa,Cf,YZx),(Xa,Cf,YZy),(Xa,Cf,YZz),(Xa,Cf,YZaa),(Xa,Cf,YZab),(Xa,Cf,YZac),(Xa, Cf,YZad),(Xa,Cf,YZae),(Xa,Cf,YZaf),(Xa,Cf,YZag),(Xa,Cf,YZah),(Xa,Cf,YZai),(Xa, Cf,YZaj),(Xa,Cf,YZak),(Xa,Cf,YZal),(Xa,Cf,YZam),(Xa,Cf,YZan),(Xa,Cf,YZao),(Xa, Cf,YZap),(Xa,Cf,YZaq),(Xa,Cf,YZar),(Xa,Cf,YZas),(Xa,Cf,YZat),(Xa,Cf,YZau),(Xa, Cf,YZav),(Xa,Cf,YZaw),(Xa,Cf,YZax),(Xa,Cf,YZay),(Xa,Cf,YZaz),(Xa,Cf,YZba),(Xa, Cf,YZbb),(Xa,Cf,YZbc),(Xa,Cf,YZbd),(Xa,Cf,YZbe),(Xa,Cf,YZbf),(Xa,Cf,YZbg), (Xa,Cf,YZbh),(Xa,Cf,YZbi),(Xa,Cf,YZbj),(Xa,Cf,YZbk),(Xa,Cf,YZbl),(Xa,Cf,YZbm), (Xa,Cf,YZbn),(Xa,Cf,YZbo),(Xa,Cf,YZbp),(Xa,Cf,YZbq),(Xa,Cf,YZbr),(Xa,Cf,YZbs), (Xa,Cf,YZbt),(Xa,Cf,YZbu),(Xa,Cf,YZbv),(Xa,Cf,YZbw),(Xa,Cf,YZbx),(Xa,Cf,YZby), (Xa,Cf,YZbz),(Xa,Cf,YZca),(Xa,Cf,YZcb),(Xa,Cf,YZcc),(Xa,Cf,YZcd),(Xa,Cf,YZce), (Xa,Cf,YZcf),(Xa,Cf,YZcg),(Xa,Cf,YZch),(Xa,Cf,YZci),(Xa,Cf,YZcj),(Xa,Cf,YZck), (Xa,Cf,YZcl),(Xa,Cf,YZcm),(Xa,Cf,YZcn),(Xa,Cf,YZco),(Xa,Cf,YZcp),(Xa,Cf,YZcq), (Xa,Cf,YZcr),(Xa,Cf,YZcs),(Xa,Cf,YZct),(Xa,Cf,YZcu),(Xa,Cf,YZcv),(Xa,Cf,YZcw), (Xa,Cf,YZcx),(Xa,Cf,YZcy),(Xa,Cf,YZcz),(Xa,Cf,YZda),(Xa,Cf,YZdb),(Xa,Cf,Ydc), (Xa,Cf,YZdd),(Xa,Cf,YZde),(Xa,Cf,YZdf),(Xa,Cf,YZdg),(Xa,Cf,YZdh),(Xa,Cf, YZdi),(Xa,Cf,YZdj),(Xa,Cf,YZdk),(Xa,Cf,YZdl),(Xa,Cf,YZdm),(Xa,Cf,YZdn),(Xa,Cf, YZdo),(Xa,Cf,YZdp),(Xa,Cf,YZdq),(Xa,Cf,YZdr),(Xa,Cf,YZds),(Xa,Cf,Yzdt),(Xa,Cf, YZdu),(Xa,Cf,YZdv),(Xa,Cf,YZdw),(Xa,Cf,YZdx),(Xa,Cf,YZdy),(Xa,Cf,YZdz),(Xa, Cf,YZea),(Xa,Cf,YZeb),(Xa,Cf,YZec),(Xa,Cf,YZed),(Xa,Cf,YZee),(Xa,Cf,YZef),(Xa, Cf,YZeg),(Xa,Cf,YZeh),(Xa,Cf,YZei),(Xa,Cf,YZej),(Xa,Cf,YZek),(Xa,Cf,YZel),(Xa, Cf,YZem),(Xa,Cf,YZen),(Xa,Cf,YZeo),(Xa,Cf,YZep),(Xa,Cf,YZeq),(Xa,Cf,YZer),(Xa, Cf,YZes),(Xa,Cf,YZet),(Xa,Cf,YZeu),(Xa,Cf,YZev),(Xa,Cf,YZew),(Xa,Cf,YZex),(Xa, Cf,YZey),(Xa,Cf,YZez),(Xa,Cf,YZfa),(Xa,Cf,YZfb),(Xa,Cf,YZfe),(Xa,Cf,YZfd),(Xa, Cf,YZfe),(Xa,Cf,YZff),(Xa,Cf,YZfg),(Xa,Cf,YZfh),(Xa,Cf,YZfi),(Xa,Cf,YZfj),(Xa, Cf,YZfk),(Xa,Cf,YZfl),(Xa,Cf,YZfm),(Xa,Cf,YZfn),(Xa,Cf,YZfo),(Xa,Cf,YZfp),(Xa, Cf,YZfq),(Xa,Cf,YZfr),(Xa,Cf,YZfs),(Xa,Cf,YZft),(Xa,Cf,YZfu),(Xa,Cf,YZfv),(Xa,Cf, YZfw),(Xa,Cf,YZfx),(Xa,Cf,YZfy),(Xa,Cf,YZfz),(Xa,Cf,YZga),(Xa,Cf,YZgb),(Xa,Cf, YZgc),(Xa,Cf,YZgd),(Xa,Cf,YZge),(Xa,Cf,YZgf),(Xa,Cf,YZgg),(Xa,Cf,YZgh),(Xa,Cf, YZgi),(Xa,Cf,YZgj),(Xa,Cf,YZgk),(Xa,Cf,YZgl),(Xa,Cf,YZgm),(Xa,Cf,YZgn),(Xa, Cf,YZgo),(Xa,Cf,YZgp),(Xa,Cf,YZgq),(Xa,Cf,YZgr),(Xa,Cf,YZgs),(Xa,Cf,YZgt), (Xb,Ca,YZa),(Xb,Ca,YZb),(Xb,Ca,YZc),(Xb,Ca,YZd),(Xb,Ca,YZe),(Xb,Ca,YZf),(Xb, Ca,YZg),(Xb,Ca,YZh),(Xb,Ca,YZi),(Xb,Ca,YZj),(Xb,Ca,YZk),(Xb,Ca,YZl),(Xb,Ca,YZm), (Xb,Ca,YZn),(Xb,Ca,YZo),(Xb,Ca,YZp),(Xb,Ca,YZq),(Xb,Ca,YZr),(Xb,Ca,YZs), (Xb,Ca,YZt),(Xb,Ca,YZu),(Xb,Ca,YZv),(Xb,Ca,YZw),(Xb,Ca,YZx),(Xb,Ca,YZy),(Xb, Ca,YZz),(Xb,Ca,YZaa),(Xb,Ca,YZab),(Xb,Ca,YZac),(Xb,Ca,YZad),(Xb,Ca,YZae),(Xb, Ca,YZaf),(Xb,Ca,YZag),(Xb,Ca,YZah),(Xb,Ca,YZai),(Xb,Ca,YZaj),(Xb,Ca,YZak), (Xb,Ca,YZal),(Xb,Ca,YZam),(Xb,Ca,YZan),(Xb,Ca,YZao),(Xb,Ca,YZap),(Xb,Ca,YZaq), (Xb,Ca,YZar),(Xb,Ca,YZas),(Xb,Ca,YZat),(Xb,Ca,YZau),(Xb,Ca,YZav),(Xb,Ca, YZaw),(Xb,Ca,YZax),(Xb,Ca,YZay),(Xb,Ca,YZaz),(Xb,Ca,YZba),(Xb,Ca,YZbb),(Xb, Ca,YZbc),(Xb,Ca,YZbd),(Xb,Ca,YZbe),(Xb,Ca,YZbf),(Xb,Ca,YZbg),(Xb,Ca,YZbh),(Xb, Ca,YZbi),(Xb,Ca,YZbj),(Xb,Ca,YZbk),(Xb,Ca,YZbl),(Xb,Ca,YZbm),(Xb,Ca,YZbn), (Xb,Ca,YZbo),(Xb,Ca,YZbp),(Xb,Ca,YZbq),(Xb,Ca,YZbr),(Xb,Ca,YZbs),(Xb,Ca, YZbt),(Xb,Ca,YZbu),(Xb,Ca,YZbv),(Xb,Ca,YZbw),(Xb,Ca,YZbx),(Xb,Ca,YZby),(Xb, Ca,YZbz),(Xb,Ca,YZca),(Xb,Ca,YZcb),(Xb,Ca,YZcc),(Xb,Ca,YZcd),(Xb,Ca,YZce),(Xb, Ca,YZcf),(Xb,Ca,YZcg),(Xb,Ca,YZch),(Xb,Ca,YZci),(Xb,Ca,YZcj),(Xb,Ca,YZck), (Xb,Ca,YZcl),(Xb,Ca,YZcm),(Xb,Ca,YZcn),(Xb,Ca,YZco),(Xb,Ca,YZcp),(Xb,Ca,YZcq), (Xb,Ca,YZcr),(Xb,Ca,YZcs),(Xb,Ca,YZct),(Xb,Ca,YZcu),(Xb,Ca,YZcv),(Xb,Ca, YZcw),(Xb,Ca,YZcx),(Xb,Ca,YZcy),(Xb,Ca,YZcz),(Xb,Ca,YZda),(Xb,Ca,YZdb),(Xb, Ca,YZdc),(Xb,Ca,YZdd),(Xb,Ca,YZde),(Xb,Ca,YZdf),(Xb,Ca,YZdg),(Xb,Ca,YZdh),(Xb, Ca,YZdi),(Xb,Ca,YZdj),(Xb,Ca,YZdk),(Xb,Ca,YZdl),(Xb,Ca,YZdm),(Xb,Ca,YZdn), (Xb,Ca,YZdo),(Xb,Ca,YZdp),(Xb,Ca,YZdq),(Xb,Ca,YZdr),(Xb,Ca,YZds),(Xb,Ca, Yzdt),(Xb,Ca,YZdu),(Xb,Ca,YZdv),(Xb,Ca,YZdw),(Xb,Ca,YZdx),(Xb,Ca,YZdy),(Xb, Ca,YZdz),(Xb,Ca,YZea),(Xb,Ca,YZeb),(Xb,Ca,YZec),(Xb,Ca,YZed),(Xb,Ca,YZee),(Xb, Ca,YZef),(Xb,Ca,YZeg),(Xb,Ca,YZeh),(Xb,Ca,YZei),(Xb,Ca,YZej),(Xb,Ca,YZek), (Xb,Ca,YZel),(Xb,Ca,YZem),(Xb,Ca,YZen),(Xb,Ca,YZeo),(Xb,Ca,YZep),(Xb,Ca,YZeq), (Xb,Ca,YZer),(Xb,Ca,YZes),(Xb,Ca,YZet),(Xb,Ca,YZeu),(Xb,Ca,YZev),(Xb,Ca, YZew),(Xb,Ca,YZex),(Xb,Ca,YZey),(Xb,Ca,YZez),(Xb,Ca,YZfa),(Xb,Ca,YZfb),(Xb, Ca,YZfc),(Xb,Ca,YZfd),(Xb,Ca,YZfe),(Xb,Ca,YZff),(Xb,Ca,YZfg),(Xb,Ca,YZfh),(Xb, Ca,YZfi),(Xb,Ca,YZfj),(Xb,Ca,YZfk),(Xb,Ca,YZfl),(Xb,Ca,YZfm),(Xb,Ca,YZfn),(Xb, Ca,YZfo),(Xb,Ca,YZfp),(Xb,Ca,YZfq),(Xb,Ca,YZfr),(Xb,Ca,YZfs),(Xb,Ca,YZft),(Xb, Ca,YZfu),(Xb,Ca,YZfv),(Xb,Ca,YZfw),(Xb,Ca,YZfx),(Xb,Ca,YZfy),(Xb,Ca,YZfz),(Xb, Ca,YZga),(Xb,Ca,YZgb),(Xb,Ca,YZgc),(Xb,Ca,YZgd),(Xb,Ca,YZge),(Xb,Ca,YZgf), f),(Xb,Ca,YZgg),(Xb,Ca,YZgh),(Xb,Ca,YZgi),(Xb,Ca,YZgj),(Xb,Ca,YZgk),(Xb,Ca,YZgl), (Xb,Ca,YZgm),(Xb,Ca,YZgn),(Xb,Ca,YZgo),(Xb,Ca,YZgp),(Xb,Ca,YZgq),(Xb, Ca,YZgr),(Xb,Ca,YZgs),(Xb,Ca,YZgt),(Xb,Cb,YZa),(Xb,Cb,YZb),(Xb,Cb,YZc),(Xb, Cb,YZd),(Xb,Cb,YZe),(Xb,Cb,YZf),(Xb,Cb,YZg),(Xb,Cb,YZh),(Xb,Cb,YZi),(Xb,Cb, YZj),(Xb,Cb,YZk),(Xb,Cb,YZl),(Xb,Cb,YZm),(Xb,Cb,YZn),(Xb,Cb,YZo),(Xb,Cb,YZp), (Xb,Cb,YZq),(Xb,Cb,YZr),(Xb,Cb,YZs),(Xb,Cb,YZt),(Xb,Cb,YZu),(Xb,Cb,YZv),(Xb, Cb,YZw),(Xb,Cb,YZx),(Xb,Cb,YZy),(Xb,Cb,YZz),(Xb,Cb,YZaa),(Xb,Cb,YZab),(Xb, Cb,YZac),(Xb,Cb,YZad),(Xb,Cb,YZae),(Xb,Cb,YZaf),(Xb,Cb,YZag),(Xb,Cb,YZah), (Xb,Cb,YZai),(Xb,Cb,YZaj),(Xb,Cb,YZak),(Xb,Cb,YZal),(Xb,Cb,YZam),(Xb,Cb, YZan),(Xb,Cb,YZao),(Xb,Cb,YZap),(Xb,Cb,YZaq),(Xb,Cb,YZar),(Xb,Cb,YZas),(Xb, Cb,YZat),(Xb,Cb,YZau),(Xb,Cb,YZav),(Xb,Cb,YZaw),(Xb,Cb,YZax),(Xb,Cb,YZay), (Xb,Cb,YZaz),(Xb,Cb,YZba),(Xb,Cb,YZbb),(Xb,Cb,YZbc),(Xb,Cb,YZbd),(Xb,Cb,YZbe), (Xb,Cb,YZbf),(Xb,Cb,YZbg),(Xb,Cb,YZbh),(Xb,Cb,YZbi),(Xb,Cb,YZbj),(Xb,Cb, YZbk),(Xb,Cb,YZbl),(Xb,Cb,YZbm),(Xb,Cb,YZbn),(Xb,Cb,YZbo),(Xb,Cb,YZbp),(Xb, Cb,YZbq),(Xb,Cb,YZbr),(Xb,Cb,YZbs),(Xb,Cb,YZbt),(Xb,Cb,YZbu),(Xb,Cb,YZbv), (Xb,Cb,YZbw),(Xb,Cb,YZbx),(Xb,Cb,YZby),(Xb,Cb,YZbz),(Xb,Cb,YZca),(Xb,Cb, YZcb),(Xb,Cb,YZcc),(Xb,Cb,YZcd),(Xb,Cb,YZce),(Xb,Cb,YZcf),(Xb,Cb,YZcg),(Xb, Cb,YZch),(Xb,Cb,YZci),(Xb,Cb,YZcj),(Xb,Cb,YZck),(Xb,Cb,YZcl),(Xb,Cb,YZcm),(Xb, Cb,YZcn),(Xb,Cb,YZco),(Xb,Cb,YZcp),(Xb,Cb,YZcq),(Xb,Cb,YZcr),(Xb,Cb,YZcs), (Xb,Cb,YZct),(Xb,Cb,YZcu),(Xb,Cb,YZcv),(Xb,Cb,YZcw),(Xb,Cb,YZcx),(Xb,Cb, YZcy),(Xb,Cb,YZcz),(Xb,Cb,YZda),(Xb,Cb,YZdb),(Xb,Cb,YZdc),(Xb,Cb,YZdd),(Xb, Cb,YZde),(Xb,Cb,YZdf),(Xb,Cb,YZdg),(Xb,Cb,YZdh),(Xb,Cb,YZdi),(Xcb,Cb,YZdj),(Xb, Cb,YZdk),(Xb,Cb,YZdl),(Xb,Cb,YZdm),(Xb,Cb,YZdn),(Xb,Cb,YZdo),(Xb,Cb,YZdp), (Xb,Cb,YZdq),(Xb,Cb,YZdr),(Xb,Cb,YZds),(Xb,Cb,Yzdt),(Xb,Cb,YZdu),(Xb,Cb, YZdv),(Xb,Cb,YZdw),(Xb,Cb,YZdx),(Xb,Cb,YZdy),(Xb,Cb,YZdz),(Xb,Cb,YZea),(Xb, Cb,YZeb),(Xb,Cb,YZec),(Xb,Cb,YZed),(Xb,Cb,YZee),(Xb,Cb,YZef),(Xb,Cb,YZeg), (Xb,Cb,YZeh),(Xb,Cb,YZei),(Xb,Cb,YZej),(Xb,Cb,YZek),(Xb,Cb,YZel),(Xb,Cb,YZem), (Xb,Cb,YZen),(Xb,Cb,YZeo),(Xb,Cb,YZep),(Xb,Cb,YZeq),(Xb,Cb,YZer),(Xb,Cb, YZes),(Xb,Cb,YZet),(Xb,Cb,YZeu),(Xb,Cb,YZev),(Xb,Cb,YZew),(Xb,Cb,YZex),(Xb, Cb,YZey),(Xb,Cb,YZez),(Xb,Cb,YZfa),(Xb,Cb,YZfb),(Xb,Cb,YZfe),(Xb,Cb,YZfd),(Xb, Cb,YZfe),(Xb,Cb,YZff),(Xb,Cb,YZfg),(Xb,Cb,YZfhl),(Xb,Cb,YZfi),(Xb,Cb,YZij), (Xb,Cb,YZfk),(Xb,Cb,YZfl),(Xb,Cb,YZfm),(Xb,Cb,YZfn),(Xb,Cb,YZfo),(Xb,Cb,YZfo), (Xb,Cb,YZfq),(Xb,Cb,YZfr),(Xb,Cb,YZfs),(Xb,Cb,YZft),(Xb,Cb,YZfu),(Xb,Cb,YZfv), (Xb,Cb,YZfw),(Xb,Cb,YZfx),(Xb,Cb,YZfy),(Xb,Cb,YZfz),(Xb,Cb,YZga),(Xb,Cb, YZgb),(Xb,Cb,YZgc),(Xb,Cb,YZgd),(Xb,Cb,YZge),(Xb,Cb,yZgf),(Xb,Cb,YZgg),(Xb, Cb,YZgh),(Xb,Cb,YZgi),(Xb,Cb,YZgj),(Xb,Cb,YZgk),(Xb,Cb,YZgl),(Xb,Cb,YZgm), (Xb,Cb,YZgn),(Xb,Cb,YZgo),(Xb,Cb,YZgp),(Xb,Cb,YZgq),(Xb,Cb,YZgr),(Xb,Cb,YZgs), (Xb,Cb,YZgt),(Xb,Cc,YZa),(Xb,Cc,YZb),(Xb,Cc,YZc),(Xb,Cc,YZd),(Xb,Cc,YZe), (Xb,Cc,YZf),(Xb,Cc,YZg),(Xb,Cc,YZh),(Xb,Cc,YZi),(Xb,Cc,YZj),(Xb,Cc,YZk),(Xb, Cc,YZl),(Xb,Cc,YZm),(Xb,Cc,YZn),(Xb,Cc,YZo),(Xb,Cc,YZp),(Xb,Cc,YZq),(Xb,Cc, YZr),(Xb,Cc,YZs),(Xb,Cc,YZt),(Xb,Cc,YZu),(Xb,Cc,YZv),(Xb,Cc,YZw),(Xb,Cc,YZx), (Xb,Cc,YZy),(Xb,Cc,YZz),(Xb,Cc,YZaa),(Xb,Cc,YZab),(Xb,Cc,YZac),(Xb,Cc,YZad), (Xb,Cc,YZae),(Xb,Cc,YZaf),(Xb,Cc,YZag),(Xb,Cc,YZah),(Xb,Cc,YZai),(Xb,Cc, YZaj),(Xb,Cc,YZak),(Xb,Cc,YZal),(Xb,Cc,YZam),(Xb,Cc,YZan),(Xb,Cc,YZao),(Xb, Cc,YZap),(Xb,Cc,YZaq),(Xb,Cc,YZar),(Xb,Cc,YZas),(Xb,Cc,YZat),(Xb,Cc,YZau),(Xb, Cc,YZav),(Xb,Cc,YZaw),(Xb,Cc,YZax),(Xb,Cc,YZay),(XbCc,YZaz),(Xb,Cc,YZba), (Xb,Cc,YZbb),(Xb,Cc,YZbc),(Xb,Cc,YZbd),(Xb,Cc,YZbe),(Xb,Cc,YZbf),(Xb,Cc,YZbg, (Xb,Cc,YZbh),(Xb,Cc,YZbi),(Xb,Cc,YZbj),(Xb,Cc,YZbk),(Xb,Cc,YZbl),(Xb,Cc, YZbm),(Xb,Cc,YZbn),(Xb,Cc,YZbo),(Xb,Cc,YZbp),(Xb,Cc,YZbq),(Xb,Cc,YZbr),(Xb, Cc,YZbs),(Xb,Cc,YZbt),(Xb,Cc,YZbu),(Xb,Cc,YZbv),(Xb,Cc,YZbw),(Xb,Cc,YZbx), (Xb,Cc,YZby),(Xb,Cc,YZbz),(Xb,Cc,YZca),(Xb,Cc,YZcb),(Xb,Cc,YZcc),(Xb,Cc,YZcd), (Xb,Cc,YZce),(Xb,Cc,YZcf),(Xb,Cc,YZcg),(Xb,Cc,YZch),(Xb,Cc,YZci),(Xb,Cc, YZcj),(Xb,Cc,YZck),(Xb,Cc,YZcl),(Xb,Cc,YZcm),(Xb,Cc,YZcn),(Xb,Cc,YZco),(Xb, Cc,YZcp),(Xb,Cc,YZcq),(Xb,Cc,YZcr),(Xb,Cc,YZcs),(Xb,Cc,YZct),(Xb,Cc,YZcu),(Xb, Cc,YZcv),(Xb,Cc,YZcw),(Xb,Cc,YZcx),(Xb,Cc,YZey),(Xb,Cc,YZcz),(Xb,Cc,YZda), (Xb,Cc,YZdb),(Xb,Cc,YZdc),(Xb,Cc,YZdd),(Xb,Cc,YZde),(Xb,Cc,YZdf),(Xb,Cc,YZdg), (Xb,Cc,YZdh),(Xb,Cc,YZdi),(Xb,Cc,YZdj),(Xb,Cc,YZdk),(Xb,Cc,YZdl),(Xb,Cc, YZdm),(Xb,Cc,YZdn),(Xb,Cc,YZdo),(Xb,Cc,YZdp),(Xb,Cc,YZdq),(Xb,Cc,YZdr),(Xb, Cc,YZds),(Xb,Cc,Yzdt),(Xb,Cc,YZdu),(Xb,Cc,YZdv),(Xb,Cc,YZdw),(Xb,Cc,YZdx), (Xb,Cc,YZdy),(Xb,Cc,YZdz),(Xb,Cc,YZea),(Xb,Cc,YZeb),(Xb,Cc,YZec),(Xb,Cc,YZed), (Xb,Cc,YZee),(Xb,Cc,YZef),(Xb,Cc,YZeg),(Xb,Cc,YZeh),(Xb,Cc,YZei),(Xb,Cc, YZej),(Xb,Cc,YZek),(Xb,Cc,YZel),(Xb,Cc,YZem),(Xb,Cc,YZen),(Xb,Cc,YZeo),(Xb, Cc,YZep),(Xb,Cc,YZeq),(Xb,Cc,YZer),(Xb,Cc,YZes),(Xb,Cc,YZet),(Xb,Cc,YZeu),(Xb, Cc,YZev),(Xb,Cc,YZew),(Xb,Cc,YZex),(Xb,Cc,YZey),(Xb,Cc,YZez),(Xb,Cc,YZfa), (Xb,Cc,YZfb),(Xb,Cc,YZfc),(Xb,Cc,YZfd),(Xb,Cc,YZfe),(Xb,Cc,YZff),(Xb,Cc,YZfg), (Xb,Cc,YZfh),(Xb,Cc,YZfi),(Xb,Cc,YZfj),(Xb,Cc,YZfk),(Xb,Cc,YZfl),(Xb,Cc,YZfm), (Xb,Cc,YZfn),(Xb,Cc,YZfo),(Xb,Cc,YZfp),(Xb,Cc,YZfq),(Xb,Cc,YZfr),(Xb,Cc,YZfs), (Xb,Cc,YZft),(Xb,Cc,YZfu),(Xb,Cc,YZfv),(Xb,Cc,YZfw),(Xb,Cc,YZfx),(Xb,Cc, YZfy),(Xb,Cc,YZfz),(Xb,Cc,YZga),(Xb,Cc,YZgb),(Xb,Cc,YZgc),(Xb,Cc,YZgd),(Xb, Cc,YZge),(Xb,Cc,YZgf),(Xb,Cc,YZgg),(Xb,Cc,YZgh),(xb,Cc,yZgi),(Xb,Cc,YZgj),(Xb, Cc,YZgk),(Xb,Cc,YZgl),(Xb,Cc,YZgm),(Xb,Cc,YZgn),(Xb,Cc,YZgo),(Xb,Cc,YZgp), (Xb,Cc,YZgq),(Xb,Cc,YZgr),(Xb,Cc,YZgs),(Xb,Cc,YZgt),(Xb,Cd,YZa),(Xb,Cd,YZb), (Xb,Cd,YZc),(Xb,Cd,YZd),(Xb,Cd,YZe),(Xb,Cd,YZf),(Xb,Cd,YZg),(Xb,Cd,YZh), (Xb,Cd,YZi),(Xb,Cd,YZj),(Xb,Cd,YZk),(Xb,Cd,YZl),(Xb,Cd,YZm),(Xb,Cd,YZn),(Xb, Cd,YZo),(Xb,Cd,YZp),(Xb,Cd,YZq),(Xb,Cd,YZr),(Xb,Cd,YZs),(Xb,Cd,YZt),(Xb,Cd, YZu),(Xb,Cd,YZv),(Xb,Cd,YZw),(Xb,Cd,YZx),(Xb,Cd,YZy),(Xb,Cd,YZz),(Xb,Cd, YZaa),(Xb,Cd,YZab),(Xb,Cd,YZac),(Xb,Cd,YZad),(Xb,Cd,YZae),(Xb,Cd,YZaf),(Xb, Cd,YZag),(Xb,Cd,YZah),(Xb,Cd,YZai),(Xb,Cd,YZaj),(Xb,Cd,YZak),(Xb,Cd,YZal),(Xb, Cd,YZam),(Xb,Cd,YZan),(Xb,Cd,YZao),(Xb,Cd,YZap),(Xb,Cd,YZaq),(Xb,Cd,YZar), (Xb,Cd,YZas),(Xb,Cd,YZat),(Xb,Cd,YZau),(Xb,Cd,YZav),(Xb,Cd,YZaw),(Xb,Cd, YZax),(Xb,Cd,YZay),(Xb,Cd,YZaz),(Xb,Cd,YZba),(Xb,Cd,YZbb),(Xb,Cd,YZbc),(Xb, Cd,YZbd),(Xb,Cd,YZbe),(Xb,Cd,YZbf),(Xb,Cd,YZbg),(Xb,Cd,YZbh),(Xb,Cd,YZbi), (Xb,Cd,YZbj),(Xb,Cd,YZbk),(Xb,Cd,YZbl),(Xb,Cd,YZbm),(Xb,Cd,YZbn),(Xb,Cd,YZbo), (Xb,Cd,YZbp),(Xb,Cd,YZbq),(Xb,Cd,YZbr),(Xb,Cd,YZbs),(Xb,Cd,YZbt),(Xb,Cd, YZbu),(Xb,Cd,YZbv),(Xb,Cd,YZbw),(Xb,Cd,YZbx),(Xb,Cd,YZby),(Xb,Cd,YZbz),(Xb, Cd,YZca),(Xb,Cd,YZcb),(Xb,Cd,YZcc),(Xb,Cd,YZcd),(Xb,Cd,YZce),(Xb,Cd,YZcf), (Xb,Cd,YZcg),(Xb,Cd,YZch),(Xb,Cd,YZci),(Xb,Cd,YZcj),(Xb,Cd,YZck),(Xb,Cd,YZcl), (Xb,Cd,YZcm),(Xb,Cd,YZcn),(Xb,Cd,YZco),(Xb,Cd,YZcp),(Xb,Cd,YZcq),(Xb, Cd,YZcr),(Xb,Cd,YZcs),(Xb,Cd,YZct),(Xb,Cd,YZcu),(Xb,Cd,YZcv),(Xb,Cd,YZcw),(Xb, Cd,YZcx),(Xb,Cd,YZcy),(Xb,Cd,YZcz),(Xb,Cd,YZda),(Xb,Cd,YZdb),(Xb,Cd,YZdc), (Xb,Cd,YZdd),(Xb,Cd,YZde),(Xb,Cd,YZdf),(Xb,Cd,YZdg),(Xb,Cd,YZdh),(Xb,Cd, YZdi),(Xb,Cd,YZdj),(Xb,Cd,YZdk),(Xb,Cd,YZdl),(Xb,Cd,YZdm),(Xb,Cd,YZdn),(Xb, Cd,YZdo),(Xb,Cd,YZdp),(Xb,Cd,YZdq),(Xb,Cd,YZdr),(Xb,Cd,YZds),(Xb,Cd,Yzdt), (Xb,Cd,YZdu),(Xb,Cd,YZdv),(Xb,Cd,YZdw),(Xb,Cd,YZdx),(Xb,Cd,YZdy),(Xb,Cd, YZdz),(Xb,Cd,YZea),(Xb,Cd,YZeb),(Xb,Cd,YZec),(Xb,Cd,YZed),(Xb,Cd,YZee),(Xb, Cd,YZef),(Xb,Cd,YZeg),(Xb,Cd,YZeh),(Xb,Cd,YZei),(Xb,Cd,YZej),(Xb,Cd,YZek),(Xb, Cd,YZel),(Xb,Cd,YZem),(Xb,Cd,YZen),(Xb,Cd,YZeo),(Xb,Cd,YZep),(Xb,Cd,YZeq), (Xb,Cd,YZer),(Xb,Cd,YZes),(Xb,Cd,YZet),(Xb,Cd,YZeu),(Xb,Cd,YZev),(Xb,Cd, YZew),(Xb,Cd,YZex),(Xb,Cd,YZey),(Xb,Cd,YZez),(Xb,Cd,YZfa),(Xb,Cd,YZfb),(Xb, Cd,YZfc),(Xb,Cd,YZfd),(Xb,Cd,YZfe),(Xb,Cd,YZff),(Xb,Cd,YZfg),(Xb,Cd,YZfh),(Xb, Cd,YZfi),(Xb,Cd,YZfj),(Xb,Cd,YZfk),(Xb,Cd,YZfl),(Xb,Cd,YZfm),(Xb,Cd,YZfn),(Xb, Cd,YZfo),(Xb,Cd,YZfp),(Xb,Cd,YZfq),(Xb,Cd,YZfr),(Xb,Cd,YZfs),(Xb,Cd,YZft), (Xb,Cd,YZfu),(Xb,Cd,YZfv),(Xb,Cd,YZfw),(Xb,Cd,YZfx),(Xb,Cd,YZfy),(Xb,Cd,YZfz), (Xb,Cd,YZga),(Xb,Cd,YZgb),(Xb,Cd,YZgc),(Xb,Cd,YZgd),(Xb,Cd,YZge),(Xb,Cd, YZgf),(Xb,Cd,YZgg),(Xb,Cd,YZgh),(Xb,Cd,YZgi),(Xb,Cd,YZgj),(Xb,Cd,YZgk),(Xb, Cd,YZgl),(Xb,Cd,YZgm),(Xb,Cd,YZgn),(Xb,Cd,YZgo),(Xb,Cd,YZgp),(Xb,Cd,YZgq), (Xb,Cd,YZgr),(Xb,Cd,YZgs),(Xb,Cd,YZgt),(Xb,Ce,YZa),(Xb,Ce,YZb),(Xb,Ce,YZc), (Xb,Ce,YZd),(Xb,Ce,YZe),(Xb,Ce,YZf),(Xb,Ce,Yg),(Xb,Ce,YZh),(Xb,Ce,YZi),(Xb, Ce,YZj),(Xb,Ce,YZk),(Xb,Ce,YZl),(Xb,Ce,YZm),(Xb,Ce,YZn),(Xb,Ce,YZo),(Xb,Ce, YZp),(Xb,Ce,YZq),(Xb,Ce,YZr),(Xb,Ce,YZs),(Xb,Ce,YZt),(Xb,Ce,YZu),(Xb,Ce,YZv), (Xb,Ce,YZw),(Xb,Ce,YZx),(Xb,Ce,YZy),(Xb,Ce,YZz),(Xb,Ce,YZaa),(Xb,Ce,YZab), (Xb,Ce,YZac),(Xb,Ce,YZad),(Xb,Ce,YZae),(Xb,Ce,YZaf),(Xb,Ce,YZag),(Xb,Ce,YZah), (Xb,Ce,YZai),(Xb,Ce,YZaj),(Xb,Ce,YZak),(Xb,Ce,YZal),(Xb,Ce,YZam),(Xb,Ce, YZan),(Xb,Ce,YZao),(Xb,Ce,YZap),(Xb,Ce,YZaq),(Xb,Ce,YZar),(Xb,Ce,YZas),(Xb, Ce,YZat),(Xb,Ce,YZau),(Xb,Ce,YZav),(Xb,Ce,YZaw),(Xb,Ce,YZax),(Xb,Ce,YZay),(Xb, Ce,YZaz),(Xb,Ce,YZba),(Xb,Ce,YZbb),(Xb,Ce,YZbc),(Xb,Ce,YZbd),(Xb,Ce,YZbe), (Xb,Ce,YZbf),(Xb,Ce,YZbg),(Xb,Ce,YZbh),(Xb,Ce,YZbi),(Xb,Ce,YZbj),(Xb,Ce,YZbk), (Xb,Ce,YZbl),(Xb,Ce,YZbm),(Xb,Ce,YZbn),(Xb,Ce,YZbo),(Xb,Ce,YZbp),(Xb, Ce,YZbq),(Xb,Ce,YZbr),(Xb,Ce,YZbs),(Xb,Ce,YZbt),(Xb,Ce,YZbu),(Xb,Ce,YZbv),(Xb, Ce,YZbw),(Xb,Ce,YZbx),(Xb,Ce,YZby),(Xb,Ce,YZbz),(Xb,Ce,YZca),(Xb,Ce,YZcb), (Xb,Ce,YZcc),(Xb,Ce,YZcd),(Xb,Ce,YZce),(Xb,Ce,YZcf),(Xb,Ce,YZcg),(Xb,Ce, YZch),(Xb,Ce,YZci),(Xb,Ce,YZcj),(Xb,Ce,YZck),(Xb,Ce,YZcl),(Xb,Ce,YZcm),(Xb, Ce,YZcn),(Xb,Ce,YZco),(Xb,Ce,YZcp),(Xb,Ce,YZcq),(Xb,Ce,YZcr),(Xb,Ce,YZcs),(Xb, Ce,YZct),(Xb,Ce,YZcu),(Xb,Ce,YZcv),(Xb,Ce,YZcw),(Xb,Ce,YZcx),(Xb,Ce,YZcy), (Xb,Ce,YZcz),(Xb,Ce,YZda),(Xb,Ce,YZdb),(Xb,Ce,YZdc),(Xb,Ce,YZdd),(Xb,Ce, YZde),(Xb,Ce,YZdf),(Xb,Ce,YZdg),(Xb,Ce,YZdh),(Xb,Ce,YZdi),(Xb,Ce,YZdj),(Xb, Ce,YZdk),(Xb,Ce,YZdl),(Xb,Ce,YZdm),(Xb,Ce,YZdn),(Xb,Ce,YZdo),(Xb,Ce,YZdp), (Xb,Ce,YZdq),(Xb,Ce,YZdr),(Xb,Ce,YZds),(Xb,Ce,Yzdt),(Xb,Ce,YZdu),(Xb,Ce,YZdv), (Xb,Ce,YZdw),(Xb,Ce,YZdx),(Xb,Ce,YZdy),(Xb,Ce,YZdz),(Xb,Ce,YZea),(Xb,Ce, YZeb),(Xb,Ce,YZec),(Xb,Ce,YZed),(Xb,Ce,YZee),(Xb,Ce,YZef),(Xb,Ce,YZeg),(Xb, Ce,YZeh),Xxb,Ce,YZei),(XbCe,eYZej),(Xb,Ce,YZek),(XbCe,eYZel),(Xb,Cw,YZem),(Xb, Ce,YZen),(Xb,Ce,YZeo),(Xb,Ce,YZep),(Xb,Ce,YZeq),(Xb,Ce,YZer),(Xb,Ce,YZes), (Xb,Ce,YZet),(Xb,Ce,YZeu),(Xb,Ce,YZev),(Xb,Ce,YZew),(Xb,Ce,YZex),(Xb,Ce,YZey), (Xb,Ce,YZez),(Xb,Ce,YZfa),(Xb,Ce,YZfb),(Xb,Ce,YZfc),(Xb,Ce,YZfd),(Xb,Ce, YZfe),(Xb,Ce,YZff),(Xb,Ce,YZfg),(Xb,Ce,YZfb),(Xb,Ce,YZfi),(Xb,Ce,YZfj),(Xb,Ce, YZfk),(Xb,Ce,YZfl),(Xb,Ce,YZfm),(Xb,Ce,YZfn),(Xb,Ce,YZfo),(Xb,Ce,YZfp),(Xb,Ce, YZfq),(Xb,Ce,YZfr),(Xb,Ce,YZfs),(Xb,Ce,YZft),(Xb,Ce,YZfu),(Xb,Ce,YZfv),(Xb,Ce, YZfw),(Xb,Ce,YZfx),(Xb,Ce,YZfy),(Xb,Ce,YZfz),(Xb,Ce,YZga),(Xb,Ce,YZgb),(Xb, Ce,YZgc),(Xb,Ce,YZgd),(Xb,Ce,YZge),(Xb,Ce,YZgf),(Xb,Ce,YZgg),(Xb,Ce,YZgh), (Xb,Ce,YZgi),(Xb,Ce,YZgj),(Xb,Ce,YZgk),(Xb,Ce,YZgl),(Xb,Ce,YZgm),(Xb,Ce,YZgn), (Xb,Ce,YZgo),(Xb,Ce,YZgp),(Xb,Ce,YZgq),(Xb,Ce,YZgr),(Xb,Ce,YZgs),(Xb,Ce, YZgt),(Xb,Cf,YZa),(Xb,Cf,YZb),(Xb,Cf,YZc),(Xb,Cf,YZd),(Xb,Cf,YZe),(Xb,Cf,YZf), (Xb,Cf,YZg),(Xb,Cf,YZh),(Xb,Cf,YZi),(Xb,Cf,YZj),(Xb,Cf,YZk),(Xb,Cf,YZl),(Xb,Cf, YZm),(Xb,Cf,YZn),(Xb,Cf,YZo),(Xb,Cf,YZp),(Xb,Cf,YZq),(Xb,Cf,YZr),(Xb,Cf,YZs), (Xb,Cf,YZt),(Xb,Cf,YZu),(Xb,Cf,YZv),(Xb,Cf,YZw),(Xb,Cf,YZx),(Xb,Cf,YZy),(Xb, Cf,YZz),(Xb,Cf,YZaa),(Xb,Cf,YZab),(Xb,Cf,YZac),(Xb,Cf,YZad),(Xb,Cf,YZae),(Xb, Cf,YZaf),(Xb,Cf,YZag),(Xb,Cf,YZah),(Xb,Cf,YZai),(Xb,Cf,YZaj),(Xb,Cf,YZak),(Xb, Cf,YZal),(Xb,Cf,YZam),(Xb,Cf,YZan),(Xb,Cf,YZao),(Xb,Cf,YZap),(Xb,Cf,YZaq),(Xb, Cf,YZar),(Xb,Cf,YZas),(Xb,Cf,YZat),(Xb,Cf,YZau),(Xb,Cf,YZav),(Xb,Cf,YZaw), (Xb,Cf,YZax),(Xb,Cf,YZay),(Xb,Cf,YZaz),(Xb,Cf,YZba),(Xb,Cf,YZbb),(Xb,Cf,YZbc), (Xb,Cf,YZbd),(Xb,Cf,YZbe),(Xb,Cf,YZbf),(Xb,Cf,YZbg),(Xb,Cf,YZbh),(Xb,Cf,YZbi), (Xb,Cf,YZbj),(Xb,Cf,YZbk),(Xb,Cf,YZbl),(Xb,Cf,YZbm),(Xb,Cf,YZbn),(Xb,Cf,YZbo), (Xb,Cf,YZbp),(Xb,Cf,YZbq),(Xb,Cf,YZbr),(Xb,Cf,YZbs),(Xb,Cf,YZbt),(Xb,Cf, YZbu),(Xb,Cf,YZbv),(Xb,Cf,YZbw),(Xb,Cf,YZbx),(Xb,Cf,YZby),(Xb,Cf,YZbz),(Xb, Cf,YZca),(Xb,Cf,YZcb),(Xb,Cf,YZcc),(Xb,Cf,YZcd),(Xb,Cf,YZce),(Xb,Cf,YZcf),(Xb, Cf,YZcg),(Xb,Cf,YZch),(Xb,Cf,YZci),(Xb,Cf,YZcj),(Xb,Cf,YZck),(Xb,Cf,YZcl),(Xb, Cf,YZcm),(Xb,Cf,YZcn),(Xb,Cf,YZco),(Xb,Cf,YZcp),(Xb,Cf,YZcq),(Xb,Cf,YZcr),(Xb, Cf,YZcs),(Xb,Cf,YZct),(Xb,Cf,YZcu),(Xb,Cf,YZcv),(Xb,Cf,YZcw),(Xb,Cf,YZcx), (Xb,Cf,YZcy),(Xb,Cf,YZcz),(Xb,Cf,YZda),(Xb,Cf,YZdb),(Xb,Cf,YZdc),(Xb,Cf,YZdd), (Xb,Cf,YZdc),(Xb,Cf,YZdf),(Xb,Cf,YZdg),(Xb,Cf,YZdh),(Xb,Cf,YZdi),(Xb,Cf,YZdj), (Xb,Cf,YZdk),(Xb,Cf,YZdl),(Xb,Cf,YZdm),(Xb,Cf,YZdn),(Xb,Cf,YZdo),(Xb,Cf, YZdp),(Xb,Cf,YZdq),(Xb,Cf,YZdr),(Xb,Cf,YZds),(Xb,Cf,Yzdt),(Xb,Cf,YZdu),(Xb,Cf, YZdv),(Xb,Cf,YZdw),(Xb,Cf,YZdx),(Xb,Cf,YZdy),(Xb,Cf,YZdz),(Xb,Cf,YZea),(Xb, Cf,YZeb),(Xb,Cf,YZec),(Xb,Cf,YZed),(XbCf,YZee),(Xb,Cf,YZef),(Xb,Cf,YZeg),(Xb, Cf,YZeh),(Xb,Cf,YZei),(Xb,Cf,YZej),(Xb,Cf,YZek),(Xb,Cf,YZel),(Xb,Cf,YZem),(Xb, Cf,YZen),(Xb,Cf,YZeo),(Xb,Cf,YZep),(Xb,Cf,YZeq),(Xb,Cf,YZer),(Xb,Cf,YZes),(Xb, Cf,YZet),(Xb,Cf,YZeu),(Xb,Cf,YZev),(Xb,Cf,YZew),(Xb,Cf,YZex),(Xb,Cf,YZey), (Xb,Cf,YZez),(Xb,Cf,YZfa),(Xb,Cf,YZfb),(Xb,Cf,YZfc),(Xb,Cf,YZfd),(Xb,Cf,YZfe), (Xb,Cf,YZff,(Xb,Cf,YZfg),(Xb,Cf,YZfh),(Xb,Cf,YZfi),(Xb,Cf,YZfj),(Xb,Cf,YZfk),(Xb, Cf,YZfl),(Xb,Cf,YZfm),(Xb,Cf,YZfn),(Xb,Cf,YZfo),(Xb,Cf,YZfp),(Xb,Cf,YZfq),(Xb, Cf,YZfr),(Xb,Cf,YZfs),(Xb,Cf,YZft),(Xb,Cf,YZfu),(Xb,Cf,YZfv),(Xb,Cf,YZfw),(Xb, Cf,YZfx),(Xb,Cf,YZfy),(Xb,Cf,YZfz),(Xb,Cf,YZga),(Xb,Cf,YZgb),(Xb,Cf,YZgc), (Xb,Cf,YZgd),(Xb,Cf,YZge),(Xb,Cf,YZgf),(Xb,Cf,YZgg),(Xb,Cf,YZgh),(Xb,Cf,YZgi), (Xb,Cf,YZgj),(Xb,Cf,YZgk),(Xb,Cf,YZgl),(Xb,Cf,YZgm),(Xb,Cf,YZgn),(Xb,Cf,YZgo), (Xb,Cf,YZgp),(Xb,Cf,YZgq),(Xb,Cf,YZgr),(Xb,Cf,YZgs),(Xb,Cf,YZgt), (Xc,Ca,YZa),(Xc,Ca,YZb),(Xc,Ca,YZc),(Xc,Ca,YZd),(Xc,Ca,YZe),(Xc,Ca,YZf),(Xc, Ca,YZg),(Xc,Ca,YZh),(Xc,Ca,YZi),(Xc,Ca,YZj),(Xc,Ca,YZk),(Xc,Ca,YZl),(Xc,Ca,YZm), (Xc,Ca,YZn),(Xc,Ca,YZo),(Xc,Ca,YZp),(Xc,Ca,YZq),(Xc,Ca,YZr),(Xc,Ca,YZs), (Xc,Ca,YZt),(Xc,Ca,YZu),(Xc,Ca,YZv),(Xc,Ca,YZw),(Xc,Ca,YZx),(Xc,Ca,YZy),(Xc, Ca,YZz),(Xc,Ca,YZaa),(Xc,Ca,YZab),(Xc,Ca,YZac),(Xc,Ca,YZad),(Xc,Ca,YZae),(Xc, Ca,YZaf),(Xc,Ca,YZag),(Xc,Ca,YZah),(cXcCa,YZai),(Xc,Ca,YZaj),(Xc,Ca,YZak),(Xc, Ca,YZal),(Xc,Ca,YZam),(Xc,Ca,YZan),(Xc,Ca,YZao),(Xc,Ca,YZap),(Xc,Ca,YZaq), (Xc,Ca,YZar),(Xc,Ca,YZas),(Xc,Ca,YZat),(Xc,Ca,YZau),(Xc,Ca,YZav),(Xc,Ca,YZaw), (Xc,Ca,YZax),(Xc,Ca,YZay),(Xc,Ca,YZaz),(Xc,Ca,YZba),(Xc,Ca,YZbb),(Xc,Ca,YZbc), (Xc,Ca,YZbd),(Xc,Ca,YZbe),(Xc,Ca,YZbf),(Xc,Ca,YZbg),(Xc,Ca,YZbh),(Xc,Ca, YZbi),(Xc,Ca,YZbj),(Xc,Ca,YZbk),(Xc,Ca,YZbl),(Xc,Ca,YZbm),(Xc,Ca,YZbn),(Xc, Ca,YZbo),(Xc,Ca,YZbp),(Xc,Ca,YZbq),(Xc,Ca,YZbr),(Xc,Ca,YZbs),(Xc,Ca,YZbt),(Xc, Ca,YZbu),(Xc,Ca,YZbv),(Xc,Ca,YZbw),(Xc,Ca,YZbx),(Xc,Ca,YZby),(Xc,Ca,YZbz), (Xc,Ca,YZca),(Xc,Ca,YZcb),(Xc,Ca,YZcc),(Xc,Ca,YZcd),(Xc,Ca,YZce),(Xc,Ca,YZcf), (Xc,Ca,YZcg),(Xc,Ca,YZch),(Xc,Ca,YZci),(Xc,Ca,YZcj),(Xc,Ca,YZck),(Xc,Ca,YZcl), (Xc,Ca,YZcm),(Xc,Ca,YZcn),(Xc,Ca,YZco),(Xc,Ca,YZcp),(Xc,Ca,YZcq),(Xc,Ca, YZer),(Xc,Ca,Ycs),(Xc,Ca,YZct),(Xc,Ca,YZcu),(Xc,Ca,YZcv),(Xc,Ca,YZcw),(Xc, Ca,YZcx),(Xc,Ca,YZcy),(Xc,Ca,YZcz),(Xc,Ca,YZda),(Xc,Ca,YZdb),(Xc,Ca,YZdc),(Xc, Ca,YZdd),(Xc,Ca,YZde),(Xc,Ca,YZdf),(Xc,Ca,YZdg),(Xc,Ca,YZdh),(Xc,Ca,YZdi), (Xc,Ca,YZdj),(Xc,Ca,YZdk),(Xc,Ca,YZdl),(Xc,Ca,YZdm),(Xc,Ca,YZdn),(Xc,Ca,YZdo), (Xc,Ca,YZdp),(Xc,Ca,YZdq),(Xc,Ca,YZdr),(Xc,Ca,YZds),(Xc,Ca,YZdt),(Xc,Ca,YZdu), (Xc,Ca,YZdv),(Xc,Ca,YZdw),(Xc,Ca,YZdx),(Xc,Ca,YZdy),(Xc,Ca,YZdz),(Xc,Ca, YZea),(Xc,Ca,YZeb),(Xc,Ca,YZec),(Xc,Ca,YZed),(Xc,Ca,YZee),(Xc,Ca,YZef),(Xc, Ca,YZeg),(Xc,Ca,YZeh),(Xc,Ca,YZei),(Xc,Ca,YZej),(Xc,Ca,YZek),(Xc,Ca,YZel),(Xc, Ca,YZem),(Xc,Ca,YZen),(Xc,Ca,YZeo),(Xc,Ca,YZep),(Xc,Ca,YZeq),(Xc,Ca,YZer),(Xc, Ca,YZes),(Xc,Ca,YZet),(Xc,Ca,YZeu),(Xc,Ca,YZev),(Xc,Ca,YZew),(Xc,Ca,YZex), (Xc,Ca,YZey),(Xc,Ca,YZez),(Xc,Ca,YZfa),(Xc,Ca,YZfb),(Xc,Ca,YZfc),(Xc,Ca,YZfd), (Xc,Ca,YZfe),(Xc,Ca,YZff),(Xc,Ca,YZfg),(Xc,Ca,YZfh),(Xc,Ca,YZfi),(Xc,Ca,YZfj), (Xc,Ca,YZfk),(Xc,Ca,YZfl),(Xc,Ca,YZfm),(Xc,Ca,YZfn),(Xc,Ca,YZfo),(Xc,Ca,YZfp), (Xc,Ca,YZfq),(Xc,Ca,YZfr),(Xc,Ca,YZfs),(Xc,Ca,YZft),(Xc,Ca,YZfu),(Xc,Ca,YZfv), (Xc,Ca,YZfw),(Xc,Ca,YZfx),(Xc,Ca,YZfy),(Xc,Ca,YZfz),(Xc,Ca,YZga),(Xc,Ca,YZgb), (Xc,Ca,YZgc),(Xc,Ca,YZgd),(Xc,Ca,YZge),(Xc,Ca,YZgf),(Xc,Ca,YZgg),(Xc,Ca, YZgh),(Xc,Ca,YZgi),(Xc,Ca,YZgj),(Xc,Ca,YZgk),(Xc,Ca,YZgl),(Xc,Ca,YZgm),(Xc, Ca,YZgn),(Xc,Ca,YZgo),(Xc,Ca,YZgp),(Xc,Ca,YZgq),(Xc,Ca,YZgr),(Xc,Ca,YZgs),(Xc, Ca,YZgt),(Xc,Cb,YZa),(Xc,Cb,YZb),(Xc,Cb,YZc),(Xc,Cb,YZd),(Xc,Cb,YZe),(Xc, Cb,YZf),(Xc,Cb,YZg),(Xc,Cb,YZh),(Xc,Cb,YZi),(Xc,Cb,YZj),(Xc,Cb,YZk),(Xc,Cb, YZl),(Xc,Cb,YZm),(Xc,Cb,YZn),(Xc,Cb,YZo),(Xc,Cb,YZp),(Xc,Cb,YZq),(Xc,Cb,YZr), (Xc,Cb,YZs),(Xc,Cb,YZt),(Xc,Cb,YZu),(Xc,Cb,YZv),(Xc,Cb,YZw),(Xc,Cb,YZx),(Xc, Cb,YZy),(Xc,Cb,YZz),(Xc,Cb,YZaa),(Xc,Cb,YZab),(Xc,Cb,YZac),(Xc,Cb,YZad), (Xc,Cb,YZae),(Xc,Cb,YZaf),(Xc,Cb,YZag),(Xc,Cb,YZah),(Xc,Cb,YZai),(Xc,Cb,YZaj), (Xc,Cb,YZak),(Xc,Cb,YZal),(Xc,Cb,YZam),(Xc,Cb,YZan),(Xc,Cb,YZao),(Xc,Cb,YXap), (Xc,Cb,YZaq),(Xc,Cb,YZar),(Xc,Cb,YZas),(Xc,Cb,YZat),(Xc,Cb,YZau),(Xc,Cb, YZav),(Xc,Cb,YZaw),(Xc,Cb,YZax),(X(c,Cb,YZay),(Xc,Cb,YZaz),(Xc,Cb,YZba),(Xc, Cb,YZbb),(Xc,Cb,YZbc),(Xc,Cb,YZbd),(Xc,Cb,YZbe),(Xc,Cb,YZbf),(Xc,Cb,YZbg),(Xc, Cb,YZbh),(Xc,Cb,YZbi),(Xc,Cb,YZbj),(Xc,Cb,YZbk),(Xc,Cb,YZbl),(Xc,Cb,YZbm), (Xc,Cb,YZbn),(Xc,Cb,YZbo),(Xc,Cb,YZbp),(Xc,Cb,YZbq),(Xc,Cb,YZbr),(Xc,Cb, YZbs),(Xc,Cb,YZbt),(Xc,Cb,YZbu),(Xc,Cb,YZbv),(Xc,Cb,YZbw),(Xc,Cb,YZbx),(Xc, Cb,YZby),(Xc,Cb,YZbz),(Xc,Cb,YZca),(Xc,Cb,YZcb),(Xc,Cb,YZcc),(Xc,Cb,Ycd),(Xc, Cb,YZce),(Xc,Cb,YZef),(Xc,Cb,YZcg),(Xc,Cb,YZch),(Xc,Cb,YZci),(Xc,Cb,YZcj), (Xc,Cb,YZck),(Xc,Cb,YZcl),(Xc,Cb,YZcm),(Xc,Cb,YZcn),(Xc,Cb,YZco),(Xc,Cb,YZcp), (Xc,Cb,YZcq),(Xc,Cb,YZcr),(Xc,Cb,YZcs),(Xc,Cb,YZct),(Xc,Cb,YZeu),(Xc,Cb, YZcv),(Xc,Cb,YZcw),(Xc,Cb,YZcx),(Xc,Cb,YZcy),(Xc,Cb,YZcz),(Xc,Cb,YZda),(Xc, Cb,YZdb),(Xc,Cb,YZdc),(Xc,Cb,YZdd),(Xc,Cb,YZde),(Xc,Cb,YZdf),(Xc,Cb,YZdg),(Xc, Cb,YZdh),(Xc,Cb,YZdi),(Xc,Cb,YZdj),(Xc,Cb,YZdk),(Xc,Cb,YZdl),(Xc,Cb,YZdm), (Xc,Cb,YZdn),(Xc,Cb,YZdo),(Xc,Cb,YZdp),(Xc,Cb,YZdq),(Xc,Cb,YZdr),(Xc,Cb, YZds),(Xc,Cb,Yzdt),(Xc,Cb,YZdu),(Xc,Cb,YZdv),(Xc,Cb,YZdw),(Xc,Cb,YZdx),(Xc, Cb,YZdy),(Xc,Cb,YZdz),(Xc,Cb,YZea),(Xc,Cb,YZeb),(Xc,Cb,YZec),(Xc,Cb,YZed),(Xc, Cb,YZee),(Xc,Cb,YZef),(Xc,Cb,YZeg),(Xc,Cb,YZeh),(Xc,Cb,YZei),(Xc,Cb,YZej), (Xc,Cb,YZek),(Xc,Cb,YZel),(Xc,Cb,YZem),(Xc,Cb,YZen),(Xc,Cb,YZeo),(Xc,Cb,YZep), (Xc,Cb,YZeq),(Xc,Cb,YZer),(Xc,Cb,YZes),(Xc,Cb,YZet),(Xc,Cb,YZeu),(Xc,Cb, YZev),(Xc,Cb,YZew),(Xc,Cb,YZex),(Xc,Cb,YZey),(Xc,Cb,YZez),(Xc,Cb,YZfa),(Xc, Cb,YZfb),(Xc,Cb,YZfc),(Xc,Cb,YZfd),(Xc,Cb,YZfe),(Xc,Cb,YZff),(Xc,Cb,YZfg),(Xc, Cb,YZfh),(Xc,Cb,YZfi),(Xc,Cb,YZfj),(Xc,Cb,YZfk),(Xc,Cb,YZfl),(Xc,Cb,YZfm),(Xc, Cb,YZfn),(Xc,Cb,YZfo),(Xc,Cb,YZfp),(Xc,Cb,YZfq),(Xc,Cb,YZfr),(Xc,Cb,YZfs),(Xc, Cb,YZft),(Xc,Cb,YZfu),(Xc,Cb,YZfv),(Xc,Cb,YZfw),(Xc,Cb,YZfx),(Xc,Cb,YZfy), (Xc,Cb,YZfz),(Xc,Cb,YZga),(Xc,Cb,YZgb),(Xc,Cb,YZgc),(Xc,Cb,YZgd),(Xc,Cb,YZge), go),(Xc,Cb,YZgf),(Xc,Cb,YZgg),(Xc,Cb,YZgh),(Xc,Cb,YZgi),(Xc,Cb,YZgj),(Xc,Cb, YZgk),(Xc,Cb,YZgl),(Xc,Cb,YZgm),(Xc,Cb,YZgn),(Xc,Cb,YZgo),(Xc,Cb,YZgp),(Xc, Cb,YZgq),(Xc,Cb,YZgr),(Xc,Cb,YZgs),(Xc,Cb,YZgt),(Xc,Cc,YZa),(Xc,Cc,YZb),(Xc, Cc,YZc),(Xc,Cc,YZd),(Xc,Cc,YZe),(Xc,Cc,YZf),(Xc,Cc,YZg),(Xc,Cc,YZh),(Xc,Cc, YZi),(Xc,Cc,YZj),(Xc,Cc,YZk),(Xc,Cc,YZl),(Xc,Cc,YZm),(Xc,Cc,YZn),(Xc,Cc,YZo), (Xc,Cc,YZp),(Xc,Cc,YZq),(Xc,Cc,YZr),(Xc,Cc,YZs),(Xc,Cc,YZt),(Xc,Cc,YZu),(Xc, Cc,YZv),(Xc,Cc,YZw),(Xc,Cc,YZx),(Xc,Cc,YZy),(Xc,Cc,YZz),(Xc,Cc,YZaa),(Xc,Ce, YZab),(Xc,Cc,YZac),(Xc,Cc,YZad),(Xc,Cc,YZae),(Xc,Cc,YZaf),(Xc,Cc,YZag),(Xc, Cc,YZah),(Xc,Cc,YZai),(Xc,Cc,YZaj),(Xc,Cc,YZak),(Xc,Cc,YZal),(Xc,Cc,YZam),(Xc, Cc,YZan),(Xc,Cc,YZao),(Xc,Cc,YZap),(Xc,Cc,YZaq),(Xc,Cc,YZar),(Xc,Cc,YZas),(Xc, Cc,YZat),(Xc,Cc,YZau),(Xc,Cc,YZav),(Xc,Cc,YZaw),(Xc,Cc,YZax),(Xc,Cc,YZay), (Xc,Cc,YZaz),(Xc,Cc,YZba),(Xc,Cc,YZbb),(Xc,Cc,YZbc),(Xc,Cc,YZbd),(Xc,Cc,YZbe), (Xc,Cc,YZbf),(Xc,Cc,YZbg),(Xc,Cc,YZbh),(Xc,Cc,YZbi),(Xc,Cc,YZbj),(Xc,Cc,YZbk), (Xc,Cc,YZbl),(Xc,Cc,YZbm),(Xc,Cc,YZbn),(Xc,Cc,YZbo),(Xc,Cc,YZbp),(Xc,Cc, YZbq),(Xc,Cc,YZbr),(Xc,Cc,YZbs),(Xc,Cc,YZbt),(Xc,Cc,YZbu),(Xc,Cc,YZbv),(Xe, Cc,YZbw),(Xc,Cc,YZbx),(Xc,Cc,YZby),(Xc,Cc,YZbz),(Xc,Cc,YZca),(Xc,Cc,YZcb),(Xc, Cc,YZcc),(Xc,Cc,YZcd),(Xc,Cc,YZce),(Xc,Cc,YZcf),(Xc,Cc,YZcg),(Xc,Cc,YZch), (Xc,Cc,YZci),(Xc,Cc,YZcj),(Xc,Cc,YZck),(Xc,Cc,YZcl),(Xc,Cc,YZcm),(Xc,Cc,YZcn), (Xc,Cc,YZco),(Xc,Cc,YZcp),(Xc,Cc,YZcq),(Xc,Cc,YZcr),(Xc,Cc,YZcs),(Xc,Cc,YZct), (Xc,Cc,YZcu),(Xc,Cc,YZcV),(Xc,Cc,YZcw),(Xc,Cc,YZcx),(Xc,Cc,YZcy),(Xc,Cc, YZcz),(Xc,Cc,YZda),(Xc,Cc,YZdb),(Xc,Cc,YZdc),(Xc,Cc,YZdd),(Xc,Cc,YZde),(Xc, Cc,YZdf),(Xc,Cc,YZdg),(Xc,Cc,YZdh),(Xc,Cc,YZdi),(Xc,Cc,YZdj),(Xc,Cc,YZdk),(Xc, Cc,YZdl),(Xc,Cc,YZdm),(Xc,Cc,YZdn),(Xc,Cc,YZdo),(Xc,Cc,YZdp),(Xc,Cc,YZdq), (Xc,Cc,YZdr),(Xc,Cc,YZds),(Xc,Cc,Yzdt),(Xc,Cc,YZdu),(Xc,Cc,YZdv),(Xc,Cc,YZdw), (Xc,Cc,YZdx),(Xc,Cc,YZdy),(Xc,Cc,YZdz),(Xc,Cc,YZea),(Xc,Cc,YZeb),(Xc,Cc, YZec),(Xc,Cc,YZed),(Xc,Cc,YZee),(Xc,Cc,YZef),(Xc,Cc,YZeg),(Xc,Cc,YZeh),(Xc,Cc, YZei),(Xc,Cc,YZej),(Xc,Cc,YZek),(Xc,Cc,YZel),(Xc,Cc,YZem),(Xc,Cc,YZen),(Xc, Cc,YZeo),(Xc,Cc,YZep),(Xc,Cc,YZeq),(Xc,Cc,YZer),(Xc,Cc,YZes),(Xc,Cc,YZet),(Xc, Cc,YZeu),(Xc,Cc,YZev),(Xc,Cc,YZew),(Xc,Cc,YZex),(Xc,Cc,YZey),(Xc,Cc,YZez), (Xc,Cc,YZfa),(Xc,Cc,YZfb),(Xc,Cc,YZfc),(Xc,Cc,YZfd),(Xc,Cc,YZfe),(Xc,Cc,YZff), (Xc,Cc,YZfg),(Xc,Cc,YZfhl),(Xc,Cc,YZfi),(Xc,Cc,YZfj),(Xc,Cc,YZfk),(Xc,Cc,YZfl),(Xc, Cc,YZfm),(Xc,Cc,YZfn),(Xc,Cc,YZfo),(Xc,Cc,YZfp),(Xc,Cc,YZfq),(Xc,Cc,YZfr), (Xc,Cc,YZfs),(Xc,Cc,YZft),(Xc,Cc,YZfu),(Xc,Cc,YZfv),(Xc,Cc,YZfw),(Xc,Cc,YZfx), (Xc,Cc,YZfy),(Xc,Cc,YZfz),(Xc,Cc,YZga),(Xc,Cc,YZgb),(Xc,Cc,YZgc),(Xc,Cc,YZgd), (Xc,Cc,YZge),(Xc,Cc,YZgf),(Xc,Cc,YZgg),(Xc,Cc,YZgh),(Xc,Cc,YZgi),(Xc,Cc,YZgj), (Xc,Cc,YZgk),(Xc,Cc,YZgl),(Xc,Cc,YZgm),(Xc,Cc,YZgn),(Xc,Cc,YZgo),(Xc,Cc, YZgp),(Xc,Cc,YZgq),(Xc,Cc,YZgr),(Xc,Cc,YZgs),(Xc,Cc,YZgt),(Xc,Cd,YZa),(Xc, Cd,YZb),(Xc,Cd,YZc),(Xc,Cd,YZd),(Xc,Cd,YZe),(Xc,Cd,YZf),(Xc,Cd,YZg),(Xc,Cd, YZh),(Xc,Cd,YZi),(Xc,Cd,YZj),(Xc,Cd,YZk),(Xc,Cd,YZl),(Xc,Cd,YZm),(Xc,Cd,YZn), (Xc,Cd,YZo),(Xc,Cd,YZp),(Xc,Cd,YZq),(Xc,Cd,YZr),(Xc,Cd,YZs),(Xc,Cd,YZt),(Xc, Cd,YZu),(Xc,Cd,YZv),(Xc,Cd,YZw),(Xc,Cd,YZx),(Xc,Cd,YZy),(Xc,Cd,YZz),(Xc, Cd,YZaa),(Xc,Cd,YZab),(Xc,Cd,YZac),(Xc,Cd,YZad),(Xc,Cd,YZae),(Xc,Cd,YZaf),(Xc, Cd,YZag),(Xc,Cd,YZah),(Xc,Cd,YZai),(Xc,Cd,YZaj),(Xc,Cd,YZak),(Xc,Cd,YZal), (Xc,Cd,YZam),(Xc,Cd,YZan),(Xc,Cd,YZao),(Xc,Cd,YZap),(Xc,Cd,YZaq),(Xc,Cd,YZar), (Xc,Cd,YZas),(Xc,Cd,YZat),(Xc,Cd,YZau),(Xc,Cd,YZav),(Xc,Cd,YZaw),(Xc,Cd, YZax),(Xc,Cd,YZay),(Xc,Cd,YZaz),(Xc,Cd,YZba),(Xc,Cd,YZbb),(Xc,Cd,YZbc),(Xc, Cd,YZbd),(Xc,Cd,YZbe),(Xc,Cd,YZbf),(Xc,Cd,YZbg),(Xc,Cd,YZbh),(Xc,Cd,YZbi),(Xc, Cd,YZbj),(Xc,Cd,YZbk),(Xc,Cd,YZbl),(Xc,Cd,YZbm),(Xc,Cd,YZbn),(Xc,Cd,YZbo), (Xc,Cd,YZbp),(Xc,Cd,YZbq),(Xc,Cd,YZbr),(Xc,Cd,YZbs),(Xc,Cd,YZbt),(Xc,Cd,YZbu), (Xc,Cd,YZbv),(Xc,Cd,YZbw),(Xc,Cd,YZbx),(Xc,Cd,YZby),(Xc,Cd,YZbz),(Xc, Cd,YZca),(Xc,Cd,YZcb),(Xc,Cd,YZcc),(Xc,Cd,YZcd),(Xc,Cd,YZce),(Xc,Cd,YZcf),(Xc, Cd,YZcg),(Xc,Cd,YZch),(Xc,Cd,YZci),(Xc,Cd,YZcj),(Xc,Cd,YZck),(Xc,Cd,YZcl), (Xc,Cd,YZcm),(Xc,Cd,YZcn),(Xc,Cd,YZco),(Xc,Cd,YZcp),(Xc,Cd,YZcq),(Xc,Cd,YZcr), (Xc,Cd,YZcs),(Xc,Cd,YZct),(Xc,Cd,YZcu),(Xc,Cd,YZcv),(Xc,Cd,YZcw),(Xc,Cd, YZcx),(Xc,Cd,YZcy),(Xc,Cd,YZcz),(Xc,Cd,YZda),(Xc,Cd,YZdb),(Xc,Cd,YZdc),(Xc, Cd,YZdd),Xxc,Cd,YZde),Xxc,Cd,YZdf),(Xc,Cd,YZdg),(Xc,Cd,YZdh),(Xc,Cd,YZdi),(Xc, Cd,YZdj),(Xc,Cd,YZdk),(Xc,Cd,YZdl),(Xc,Cd,YZdm),(Xc,Cd,YZdn),(Xc,Cd,YZdo), (Xc,Cd,YZdp),(Xc,Cd,YZdq),(Xc,Cd,YZdr),(Xc,Cd,YZds),(Xc,Cd,YZdt),(Xc,Cd,YZdu), (Xc,Cd,YZdv),(Xc,Cd,YZdw),(Xc,Cd,YZdx),(Xc,Cd,YZdy),(Xc,Cd,YZdz),(Xc, Cd,YZea),(Xc,Cd,YZeb),(Xc,Cd,YZec),(Xc,Cd,YZed),(Xc,Cd,YZee),(Xc,Cd,YZef),(Xc, Cd,YZeg),(Xc,Cd,YZeh),(Xc,Cd,YZei),(Xc,Cd,YZej),(Xc,Cd,YZek),(Xc,Cd,YZel), (Xc,Cd,YZem),(Xc,Cd,YZen),(Xc,Cd,YZeo),(Xc,Cd,YZep),(Xc,Cd,YZeq),(Xc,Cd,YZer), (Xc,Cd,YZes),(Xc,Cd,YZet),(Xc,Cd,YZeu),(Xc,Cd,YZev),(Xc,Cd,YZew),(Xc,Cd, YZex),(Xc,Cd,YZey),(Xc,Cd,YZez),(Xc,Cd,YZfa),(Xc,Cd,YZfb),(Xc,Cd,YZfc),(Xc, Cd,YZfd),(Xc,Cd,YZfe),(Xc,Cd,YZff),(Xc,Cd,YZfg),(Xc,Cd,YZfh),(Xc,Cd,YZfi),(Xc, Cd,YZfj),(Xc,Cd,YZfk),(Xc,Cd,YZfl),(Xc,Cd,YZfm),(Xc,Cd,YZfn),(Xc,Cd,YZfo),(Xc, Cd,YZfp),(Xc,Cd,YZfq),(Xc,Cd,YZfr),(Xc,Cd,YZfs),(Xc,Cd,YZft),(Xc,Cd,YZfu),(Xc, Cd,YZfv),(Xc,Cd,YZfw),(Xc,Cd,YZfx),(Xc,Cd,YZfy),(Xc,Cd,YZfz),(Xc,Cd,YZga), (Xc,Cd,YZgb),(Xc,Cd,YZgc),(Xc,Cd,YZgd),(Xc,Cd,YZge),(Xc,Cd,YZgf),(Xc,Cd,YZgg), (Xc,Cd,YZgh),(Xc,Cd,YZgi),(Xc,Cd,YZgj),(Xc,Cd,YZgk),(Xc,Cd,YZgl),(Xc,Cd, YZgm),(Xc,Cd,YZgn),(Xc,Cd,YZgo),(Xc,Cd,YZgp),(Xc,Cd,YZgq),(Xc,Cd,YZgr),(Xc, Cd,YZgs),(Xc,Cd,YZgt),(Xc,Ce,YZa),(Xc,Ce,YZb),(Xc,Ce,YZc),(Xc,Ce,YZd),(Xc,Ce, YZe),(Xc,Ce,YZf),(Xc,Ce,YZg),(Xc,Ce,YZh),(Xc,Ce,YZi),(Xc,Ce,YZj),(Xc,Ce,YZk), (Xc,Ce,YZl),(Xc,Ce,YZm),(Xc,Ce,YZn),(Xc,Ce,YZo),(Xc,Ce,YZp),(Xc,Ce,YZq),(Xc, Ce,YZr),(Xc,Ce,YZs),(Xc,Ce,YZt),(Xc,Ce,YZu),(Xc,Ce,YZv),(Xc,Ce,YZw),(Xc,Ce, YZx),(Xc,Ce,YZy),(Xc,Ce,YZz),(Xc,Ce,YZaa),(Xc,Ce,YZab),(Xc,Ce,YZac),(Xc,Ce, YZad),(Xc,Ce,YZae),(Xc,Ce,YZaf),(Xc,Ce,YZgj),(Xc,Ce,YZah),(Xc,Ce,YZai),(Xc,Ce, YZaj),(Xc,Ce,YZak),(Xc,Ce,YZal),(Xc,Ce,YZam),(Xc,Ce,YZan),(Xc,Ce,YZao),(Xc, Ce,YZap),(Xc,Ce,YZaq),(Xc,Ce,YZar),(Xc,Ce,YZas),(Xc,Ce,YZat),(Xc,Ce,YZau),(Xc, Ce,YZav),(Xc,Ce,YZaw),(Xc,Ce,YZax),(Xc,Ce,YZay),(Xc,Ce,YZaz),(Xc,Ce,YZba), (Xc,Ce,YZbb),(Xc,Ce,YZbc),(Xc,Ce,YZbd),(Xc,Ce,YZbe),(Xc,Ce,YZbf),(Xc,Ce,YZbg), (Xc,Ce,YZbh),(Xc,Ce,YZbi),(Xc,Ce,YZbj),(Xc,Ce,YZbk),(Xc,Ce,YZbl),(Xc,Ce,YZbm), (Xc,Ce,YZbn),(Xc,Ce,YZbo),(Xc,Ce,YZbp),(Xc,Ce,YZbq),(Xc,Ce,YZbr),(Xc,Ce, YZbs),(Xc,Ce,YZbt),(Xc,Ce,YZbu),(Xc,Ce,YZbv),(Xc,Ce,YZbw),(Xc,Ce,YZbx),(Xc, Ce,YZby),(Xc,Ce,YZbz),(Xc,Ce,YZca),(Xc,Ce,YZcb),(Xc,Ce,YZcc),(Xc,Ce,YZed),(Xc, Ce,YZce),(Xc,Ce,YZcf),(Xc,Ce,YZcg),(Xc,Ce,YZch),(Xc,Ce,YZci),(Xc,Ce,YZcj), (Xc,Ce,YZck),(Xc,Ce,YZcl),(Xc,Ce,YZcm),(Xc,Ce,YZcn),(Xc,Ce,YZco),(Xc,Ce,YZcp), (Xc,Ce,YZcq),(Xc,Ce,YZcr),(Xe,Ce,YZcs),(Xc,Ce,YZct),(Xc,Ce,YZcu),(Xc,Ce,YZcv), (Xc,Ce,YZcw),(Xc,Ce,YZcx),(Xc,Ce,YZcy),(Xc,Ce,YZcz),(Xc,Ce,YZda),(Xc,Ce, YZdb),(Xc,Ce,YZdc),(Xc,Ce,YZdd),(Xc,Ce,YZde),(Xc,Ce,YZdf),(Xc,Ce,YZdg),(Xc, Ce,YZdh),(Xc,Ce,YZdi),(Xc,Ce,YZdj),(Xc,Ce,YZdk),(Xc,Ce,YZdl),(Xc,Ce,YZdm),(Xc, Ce,YZdn),(Xc,Ce,YZdo),(Xc,Ce,YZdp),(Xc,Ce,YZdq),(Xc,Ce,YZdr),(Xc,Ce,YZds), (Xc,Ce,Yzdt),(Xc,Ce,YZdu),(Xc,Ce,YZdv),(Xc,Ce,YZdw),(Xc,Ce,YZdx),(Xc,Ce,YZdy), (Xc,Ce,YZdz),(Xc,Ce,YZea),(Xc,Ce,YZeb),(Xc,Ce,YZec),(Xc,Ce,YZed),(Xc,Ce, YZee),(Xc,Ce,YZef),(Xc,Ce,YZeg),(Xc,Ce,YZeh),(Xc,Ce,YZei),(Xc,Ce,YZej),(Xc,Ce, YZek),(Xc,Ce,YZel),(Xc,Ce,YZem),(Xc,Ce,YZen),(Xc,Ce,YZeo),(Xc,Ce,YZep),(Xc, Ce,YZeq),(Xc,Ce,YZer),(Xc,Ce,YZes),(Xc,Ce,YZet),(Xc,Ce,YZeu),(Xc,Ce,YZev),(Xc, Ce,YZew),(Xc,Ce,YZex),(Xc,Ce,YZey),(Xc,Ce,YZez),(Xc,Ce,YZfa),(Xc,Ce,YZfb),(Xc, Ce,YZfc),(Xc,Ce,YZfd),(Xc,Ce,YZfe),(Xc,Ce,YZff),(Xc,Ce,YZfg),(Xc,Ce,YZfh),(Xc, Ce,YZfi),(Xc,Ce,YZfj),(Xc,Ce,YZfk),(Xc,Ce,YZfl),(Xc,Ce,YZfm),(Xc,Ce,YZfn),(Xc, Ce,YZfo),(Xc,Ce,YZfp),(Xc,Ce,YZfq),(Xc,Ce,YZfr),(Xc,Ce,YZfs),(Xc,Ce,YZft),(Xc, Ce,YZfu),(Xc,Ce,YZfv),(Xc,Ce,YZfw),(Xc,Ce,YZfx),(Xc,Ce,YZfy),(Xc,Ce,YZfz), (Xc,Ce,YZga),(Xc,Ce,YZgb),(Xc,Ce,YZgc),(Xc,Ce,YZgd),(Xc,Ce,YZge),(Xc,Ce,YZgf), (Xc,Ce,YZgg),(Xc,Ce,YZgh),(Xc,Ce,YZgi),(Xc,Ce,YZgj),(Xc,Ce,YZgk),(Xc,Ce,YZgl), (Xc,Ce,YZgm),(Xc,Ce,YZgn),(Xc,Ce,YZgo),(Xc,Ce,YZgp),(Xc,Ce,YZgq),(Xc,Ce, YZgr),(Xc,Ce,YZgs),(Xc,Ce,YZgt),(Xc,Cf,YZa),(Xc,Cf,YZb),(Xc,Cf,YZc),(Xc,Cf, YZd),(Xc,Cf,YZe),(Xc,Cf,YZf),(Xc,Cf,YZg),(Xc,Cf,YZh),(Xc,Cf,YZi),(Xc,Cf,YZj),(Xc, Cf,YZk),(Xc,Cf,YZl),(Xc,Cf,YZm),(Xc,Cf,YZn),(Xc,Cf,YZo),(Xc,Cf,YZp),(Xc,Cf, YZq),(Xc,Cf,YZr),(Xc,Cf,YZs),(Xc,Cf,YZt),(Xc,Cf,YZu),(Xc,Cf,YZv),(Xc,Cf,YZw), (Xc,Cf,YZx),(Xc,Cf,YZy),(Xc,Cf,YZz),(Xc,Cf,YZaa),(Xc,Cf,YZab),(Xc,Cf,YZac),(Xc, Cf,YZad),(Xc,Cf,YZae),(Xc,Cf,YZaf),(Xc,Cf,YZag),(Xc,Cf,YZah),(Xc,Cf,YZai),(Xc, Cf,YZaj),(Xc,Cf,YZak),(Xc,Cf,YZal),(Xc,Cf,YZam),(Xc,Cf,YZan),(Xc,Cf,YZao),(Xc, Cf,YZap),(Xc,Cf,YZaq),(Xc,Cf,YZar),(Xc,Cf,YZas),(Xc,Cf,YZat),(Xc,Cf,YZau),(Xc, Cf,YZav),(Xc,Cf,YZaw),(Xc,Cf,YZax),(Xc,Cf,YZay),(Xc,Cf,YZaz),(Xc,Cf,YZba),(Xc, Cf,YZbb),(Xc,Cf,YZbc),(Xc,Cf,YZbd),(Xc,Cf,YZbe),(Xc,Cf,YZbf),(Xc,Cf,YZbg), (Xc,Cf,YZbh),(Xc,Cf,YZbi),(Xc,Cf,YZbj),(Xc,Cf,YZbk),(Xc,Cf,YZbl),(Xc,Cf,YZbm), (Xc,Cf,YZbn),(Xc,Cf,YZbo),(Xc,Cf,YZbp),(Xc,Cf,YZbq),(Xc,Cf,YZbr),(Xc,Cf,YZbs), (Xc,Cf,YZbt),(Xc,Cf,YZbu),(Xc,Cf,YZbv),(Xc,Cf,YZbw),(Xc,Cf,YZbx),(Xc,Cf,YZby), (Xc,Cf,YZbz),(Xc,Cf,YZca),(Xc,Cf,YZcb),(Xc,Cf,YZcc),(Xc,Cf,YZcd),(Xc,Cf,YZce), (Xc,Cf,YZcf),(Xc,Cf,YZcg),(Xc,Cf,YZch),(Xc,Cf,YZci),(Xc,Cf,YZcj),(Xc,Cf,YZck), (Xc,Cf,YZcl),(Xc,Cf,YZcm),(Xc,Cf,YZcn),(Xc,Cf,YZco),(Xc,Cf,YZcp),(Xc,Cf,YZcq), (Xc,Cf,YZcr),(Xc,Cf,YZes),(Xc,Cf,YZct),(Xc,Cf,YZeu),(Xc,Cf,YZcv),(Xc,Cf,YZcw), (Xc,Cf,YZcx),(Xc,Cf,YZcy),(Xc,Cf,YZcz),(Xc,Cf,YZda),(Xc,Cf,YZdb),(Xc,Cf,YZdc), (Xc,Cf,YZdd),(Xc,Cf,YZde),(xXcCf,YZdf),(Xc,Cf,YZdg),(Xc,Cf,YZdh),(Xc,Cf, YZdi),(Xc,Cf,YZdj),(Xc,Cf,YZdk),(Xc,Cf,YZdl),(Xc,Cf,YZdm),(Xc,Cf,YZdn),(Xc,Cf, YZdo),(Xc,Cf,YZdp),(Xc,Cf,YZdq),(Xc,Cf,YZdr),(Xc,Cf,YZds),(Xc,Cf,Yzdt),(Xc,Cf, YZdu),(Xc,Cf,YZdv),(Xc,Cf,YZdw),(Xc,Cf,YZdx),(Xc,Cf,YZdy),(Xc,Cf,YZdz),(Xc, Cf,YZea),(Xc,Cf,YZeb),(Xc,Cf,YZec),(Xc,Cf,YZed),(Xc,Cf,YZee),(Xc,Cf,YZef),(Xc, Cf,YZeg),(Xc,Cf,YZeh),(Xc,Cf,YZei),(Xc,Cf,YZej),(Xc,Cf,YZek),(Xc,Cf,YZel),(Xc, Cf,YZem),(Xc,Cf,YZen),(Xc,Cf,YZeo),(Xc,Cf,YZep),(Xc,Cf,YZeq),(Xc,Cf,YZer),(Xc, Cf,YZes),(Xc,Cf,YZet),(Xc,Cf,YZeu),(Xc,Cf,YZev),(Xc,Cf,YZew),(Xc,Cf,YZex),(Xc, Cf,YZey),(Xc,Cf,YZez),(Xc,Cf,YZfa),(Xc,Cf,YZfb),(Xc,Cf,YZfc),(Xc,Cf,YZfd),(Xc, Cf,YZfe),(Xc,Cf,YZff),(Xc,Cf,YZfg),(Xc,Cf,YZfh),(Xc,Cf,YZfi),(Xc,Cf,YZaj,),(Xc, Cf,YZfk),(Xc,Cf,YZfl),(Xc,Cf,YZfm),(Xc,Cf,YZfn),(Xc,Cf,YZfo),(Xc,Cf,YZfp),(Xc, Cf,YZfq),(Xc,Cf,YZfr),(Xc,Cf,YZfs),(Xc,Cf,YZft),(Xc,Cf,YZfu),(Xc,Cf,YZfv),(Xc,Cf, YZfw),(Xc,Cf,YZfx),(Xc,Cf,YZfy),(Xc,Cf,YZfz),(Xc,Cf,YZga),(Xc,Cf,YZgb),(Xc,Cf, YZgc),(Xc,Cf,YZgd),(Xc,Cf,YZge),(Xc,Cf,YZgf),(Xc,Cf,YZgg),(Xc,Cf,YZgh),(Xc,Cf, YZgi),(Xc,Cf,YZgj),(Xc,Cf,YZgk),(Xc,Cf,YZgl),(Xc,Cf,YZgm),(Xc,Cf,YZgn),(Xc, Cf,YZgo),(Xc,Cf,YZgp),(Xc,Cf,YZgq),(Xc,Cf,YZgr),(Xc,Cf,YZgs),(Xc,Cf,YZgt), (Xd,Ca,YZa),(Xd,Ca,YZb),(Xd,Ca,YZc),(Xd,Ca,YZd),(Xd,Ca,YZe),(Xd,Ca,YZf),(Xd, Ca,YZg),(Xd,Ca,YZh),(Xd,Ca,YZi),(Xd,Ca,YZj),(Xd,Ca,YZk),(Xd,Ca,YZl),(Xd,Ca,YZm), (Xd,Ca,YZn),(Xd,Ca,YZo),(Xd,Ca,YZp),(Xd,Ca,YZq),(Xd,Ca,YZr),(Xd,Ca,YZs), (Xd,Ca,YZt),(Xd,Ca,YZu),(Xd,Ca,YZv),(Xd,Ca,YZw),(Xd,Ca,YZx),(Xd,Ca,YZy),(Xd, Ca,YZz),(Xd,Ca,YZaa),(Xd,Ca,YZab),(Xd,Ca,YZac),(Xd,Ca,YZad),(Xd,Ca,YZae),(Xd, Ca,YZaf),(Xd,Ca,YZag),(Xd,Ca,YZah),(Xd,Ca,YZai),(Xd,Ca,YZaj),(Xd,Ca,YZak), (Xd,Ca,YZal),(Xd,Ca,YZam),(Xd,Ca,YZan),(Xd,Ca,YZao),(Xd,Ca,YZap),(Xd,Ca,YZaq), (Xd,Ca,YZar),(Xd,Ca,YZas),(Xd,Ca,YZat),(Xd,Ca,YZau),(Xd,Ca,YZav),(Xd,Ca, YZaw),(Xd,Ca,YZax),(Xd,Ca,YZay),(Xd,Ca,YZaz),(Xd,Ca,YZba),(Xd,Ca,YZbb),(Xd, Ca,YZbc),(Xd,Ca,YZbd),(Xd,Ca,YZbe),(Xd,Ca,YZbf),(Xd,Ca,YZbg),(Xd,Ca,YZbh),(Xd, Ca,YZbi),(Xd,Ca,YZbj),(Xd,Ca,YZbk),(Xd,Ca,YZbl),(Xd,Ca,YZbm),(Xd,Ca,YZbn), (Xd,Ca,YZbo),(Xd,Ca,YZbp),(Xd,Ca,YZbq),(Xd,Ca,YZbr),(Xd,Ca,YZbs),(Xd,Ca, YZbt),(Xd,Ca,YZbu),(Xd,Ca,YZbv),(Xd,Ca,YZbw),(Xd,Ca,YZbx),(Xd,Ca,YZby),(Xd, Ca,YZbz),(Xd,Ca,YZca),(Xd,Ca,YZcb),(Xd,Ca,YZcc),(Xd,Ca,YZcd),(Xd,Ca,YZce),(Xd, Ca,Yscf),(Xd,Ca,YZcg),(Xd,Ca,YZch),(Xd,Ca,YZci),(Xd,Ca,YZcj),(Xd,Ca,YZck), (Xd,Ca,YZel),(Xd,Ca,YZcm),(Xd,Ca,YZcn),(Xd,Ca,YZco),(Xd,Ca,YZcp),(Xd,Ca,YZcq), (Xd,Ca,YZcr),(Xd,Ca,YZes),(Xd,Ca,YZct),(Xd,Ca,YZcu),(Xd,Ca,YZcv),(Xd,Ca, YZcw),(Xd,Ca,YZcx),(Xd,Ca,YZcy),(Xd,Ca,YZcz),(Xd,Ca,YZda),(Xd,Ca,YZdb),(Xd, Ca,YZdc),(Xd,Ca,YZdd),(Xd,Ca,YZde),(Xd,Ca,YZdf),(Xd,Ca,YZdg),(Xd,Ca,YZdh),(Xd, Xd,Ca,YZdi),(Xd,Ca,YZdj),(Xd,Ca,YZdk),(Xd,Ca,YZdl),(Xd,Ca,YZdm),(Xd,Ca,YZdn), (Xd,Ca,YZdo),(Xd,Ca,YZdp),(Xd,Ca,YZdq),(Xd,Ca,YZdr),(Xd,Ca,YZds),(Xd,Ca, Yzdt),(Xd,Ca,YZdu),(Xd,Ca,YZdv),(Xd,Ca,YZdw),(Xd,Ca,YZdx),(Xd,Ca,YZdy),(Xd, Ca,YZdz),(Xd,Ca,YZea),(Xd,Ca,YZeb),(Xd,Ca,YZec),(Xd,Ca,YZed),(Xd,Ca,YZee),(Xd, Ca,YZef),(Xd,Ca,YZeg),(Xd,Ca,YZeh),(Xd,Ca,YZei),(Xd,Ca,YZej),(Xd,Ca,YZek), (Xd,Ca,YZel),(Xd,Ca,YZem),(Xd,Ca,YZen),(Xd,Ca,YZeo),(Xd,Ca,YZep),(Xd,Ca,YZeq), (Xd,Ca,YZer),(Xd,Ca,YZes),(Xd,Ca,YZet),(Xd,Ca,YZeu),(Xd,Ca,YZev),(Xd,Ca, YZew),(Xd,Ca,YZex),(Xd,Ca,YZey),(Xd,Ca,YZez),(Xd,Ca,YZfa),(Xd,Ca,YZfb),(Xd, Ca,YZfc),(Xd,Ca,YZfd),(Xd,Ca,YZfe),(Xd,Ca,YZff),(Xd,Ca,YZfg),(Xd,Ca,YZfh),(Xd, Ca,YZfi),(Xd,Ca,YZfj),(Xd,Ca,YZfk),(Xd,Ca,YZfl),(Xd,Ca,YZfm),(Xd,Ca,YZfn),(Xd, Ca,YZfo),(Xd,Ca,YZfp),(Xd,Ca,YZfq),(Xd,Ca,YZfr),(Xd,Ca,YZfs),(Xd,Ca,YZft),(Xd, Ca,YZfu),(Xd,Ca,YZfv),(Xd,Ca,YZfw),(Xd,Ca,YZfx),(Xd,Ca,YZfy),(Xd,Ca,YZfz),(Xd, Ca,YZga),(Xd,Ca,YZgb),(Xd,Ca,YZgc),(Xd,Ca,YZgd),(Xd,Ca,YZge),(Xd,Ca,YZgf), (Xd,Ca,YZgg),(Xd,Ca,YZgh),(Xd,Ca,YZgi),(Xd,Ca,YZgj),(Xd,Ca,YZgk),(Xd,Ca,YZgl), (Xd,Ca,YZgm),(Xd,Ca,YZgn),(Xd,Ca,YZgo),(Xd,Ca,YZgp),(Xd,Ca,YZgq),(Xd, Ca,YZgr),(Xd,Ca,YZgs),(Xd,Ca,YZgt),(Xd,Cb,YZa),(Xd,Cb,YZb),(Xd,Cb,YZc),(Xd, Cb,YZd),(Xd,Cb,YZe),(Xd,Cb,YZf),(Xd,Cb,YZg),(Xd,Cb,YZh),(Xd,Cb,YZi),(Xd,Cb, YZj),(Xd,Cb,YZk),(Xd,Cb,YZl),(Xd,Cb,YZm),(Xd,Cb,YZn),(Xd,Cb,YZo),(Xd,Cb,YZp), (Xd,Cb,YZq),(Xd,Cb,YZr),(Xd,Cb,YZs),(Xd,Cb,YZt),(Xd,Cb,YZu),(Xd,Cb,YZv),(Xd, Cb,YZw),(Xd,Cb,YZx),(Xd,Cb,YZy),(Xd,Cb,YZz),(Xd,Cb,YZaa),(Xd,Cb,YZab),(Xd, Cb,YZac),(Xd,Cb,YZad),(Xd,Cb,YZae),(Xd,Cb,YZaf),(Xd,Cb,YZag),(Xd,Cb,YZah), (Xd,Cb,YZai),(Xd,Cb,YZaj),(Xd,Cb,YZak),(Xd,Cb,YZal),(Xd,Cb,YZam),(Xd,Cb, YZan),(Xd,Cb,YZao),(Xd,Cb,YZap),(Xd,Cb,YZaq),(Xd,Cb,YZar),(Xd,Cb,YZas),(Xd, Cb,YZat),(Xd,Cb,YZau),(Xd,Cb,YZav),(Xd,Cb,YZaw),(Xd,Cb,YZax),(Xd,Cb,YZay), (Xd,Cb,YZaz),(Xd,Cb,YZba),(Xd,Cb,YZbb),(Xd,Cb,YZbc),(Xd,Cb,YZbd),(Xd,Cb,YZbe), (Xd,Cb,YZbf),(Xd,Cb,YZbg),(Xd,Cb,YZbh),(Xd,Cb,YZbi),(Xd,Cb,YZbj),(Xd,Cb, YZbk),(Xd,Cb,YZbl),(Xd,Cb,YZbm),(Xd,Cb,YZbn),(Xd,Cb,YZbo),(Xd,Cb,YZbp),(Xd, Cb,YZbq),(Xd,Cb,YZbr),(Xd,Cb,YZbs),(Xd,Cb,YZbt),(Xd,Cb,YZbu),(Xd,Cb,YZbv), (Xd,Cb,YZbw),(Xd,Cb,YZbx),(Xd,Cb,YZby),(Xd,Cb,YZbz),(Xd,Cb,YZca),(Xd,Cb, YZeb),(Xd,Cb,YZcc),(Xd,Cb,YZcd),(Xd,Cb,YZce),(Xd,Cb,YZcf),(Xd,Cb,YZcg),(Xd, Cb,YZch),(Xd,Cb,YZci),(Xd,Cb,YZcj),(Xd,Cb,YZck),(Xd,Cb,YZcl),(Xd,Cb,YZcm),(Xd, Cb,YZcn),(Xd,Cb,YZco),(Xd,Cb,YZcp),(Xd,Cb,YZcq),(Xd,Cb,YZcr),(Xd,Cb,YZcs), (Xd,Cb,YZct),(Xd,Cb,YZcu),(Xd,Cb,YZcv),(Xd,Cb,YZcw),(Xd,Cb,YZcx),(Xd,Cb, YZcy),(Xd,Cb,YZcz),(Xd,Cb,YZda),(Xd,Cb,YZdb),(Xd,Cb,YZdc),(Xd,Cb,YZdd),(Xd, Cb,YZde),(Xd,Cb,YZdf),(Xd,Cb,YZdg),(Xd,Cb,YZdh),(Xd,Cb,YZdi),(Xd,Cb,YZdj),(Xd, Cb,YZdk),(Xd,Cb,YZdl),(Xd,Cb,YZdm),(Xd,Cb,YZdn),(Xd,Cb,YZdo),(Xd,Cb,YZdp), (Xd,Cb,YZdq),(Xd,Cb,YZdr),(Xd,Cb,YZds),(Xd,Cb,Yzdt),(Xd,Cb,YZdu),(Xd,Cb, YZdv),(Xd,Cb,YZdw),(Xd,Cb,YZdx),(Xd,Cb,YZdy),(Xd,Cb,YZdz),(Xd,Cb,YZea),(Xd, Cb,YZeb),(Xd,Cb,YZec),(Xd,Cb,YZed),(Xd,Cb,YZee),(Xd,Cb,YZef),(Xd,Cb,YZeg), (Xd,Cb,YZeh),(Xd,Cb,YZei),(Xd,Cb,YZej),(Xd,Cb,YZek),(Xd,Cb,YZel),(Xd,Cb,YZem), (Xd,Cb,YZen),(Xd,Cb,YZeo),(Xd,Cb,YZep),(Xd,Cb,YZeq),(Xd,Cb,YZer),(Xd,Cb, YZes),(Xd,Cb,YZet),(Xd,Cb,YZeu),(Xd,Cb,YZev),(Xd,Cb,YZew),(Xd,Cb,YZex),(Xd, Cb,YZey),(Xd,Cb,YZez),(Xd,Cb,YZfa),(Xd,Cb,YZfb),(Xd,Cb,YZfc),(Xd,Cb,YZfd),(Xd, Cb,YZfe),(Xd,Cb,YZff),(Xd,Cb,YZfg),(Xd,Cb,YZfh),(Xd,Cb,YZfi),(Xd,Cb,YZfj), (Xd,Cb,YZfk),(Xd,Cb,YZfl),(Xd,Cb,YZfm),(Xd,Cb,YZfn),(Xd,Cb,YZfo),(Xd,Cb,YZfp), (Xd,Cb,YZfq),(Xd,Cb,YZfr),(Xd,Cb,YZfs),(Xd,Cb,YZft),(Xd,Cb,YZfu),(Xd,Cb,YZfv), (Xd,Cb,YZfw),(Xd,Cb,YZfx),(Xd,Cb,YZfy),(Xd,Cb,YZfz),(Xd,Cb,YZga),(Xd,Cb, YZgb),(Xd,Cb,YZgc),(Xd,Cb,YZgd),(Xd,Cb,YZge),(Xd,Cb,YZgf),(Xd,Cb,YZgg),(Xd, Cb,YZgh),(Xd,Cb,YZgi),(Xd,Cb,YZgj),(Xd,Cb,YZgk),(Xd,Cb,YZgl),(Xd,Cb,YZgm), (Xd,Cb,YZgn),(Xd,Cb,YZgo),(Xd,Cb,YZgp),(Xd,Cb,YZgq),(Xd,Cb,YZgr),(Xd,Cb,YZgs), (Xd,Cb,YZgt),(Xd,Cc,YZa),(Xd,Cc,YZb),(Xd,Cc,YZc),(Xd,Cc,YZd),(Xd,Cc,YZe), (Xd,Cc,YZf),(Xd,Cc,YZg),(Xd,Cc,YZh),(Xd,Cc,YZi),(Xd,Cc,YZj),(Xd,Cc,YZk),(Xd, Cc,YZl),(Xd,Cc,YZm),(Xd,Cc,YZn),(Xd,Cc,YZo),(Xd,Cc,YZp),(Xd,Cc,YZq),(Xd,Cc, YZr),(Xd,Cc,Yss),(Xd,Cc,YZt),(Xd,Cc,YZu),(Xd,Cc,YZv),(Xd,Cc,YZw),(Xd,Cc,YZx), (Xd,Cc,YZy),(Xd,Cc,YZz),(Xd,Cc,YZaa),(Xd,Cc,YZab),(Xd,Cc,YZac),(Xd,Cc,YZad), (Xd,Cc,YZae),(Xd,Cc,YZaf),(Xd,Cc,YZag),(Xd,Cc,YZah),(Xd,Cc,YZai),(Xd,Cc, YZaj),(Xd,Cc,YZak),(Xd,Cc,YZal),(Xd,Cc,YZam),(Xd,Cc,YZan),(Xd,Cc,YZao),(Xd, Cc,YZap),(Xd,Cc,YZaq),(Xd,Cc,YZar),(Xd,Cc,YZas),(Xd,Cc,YZat),(Xd,Cc,YZau),(Xd, Cc,YZav),(Xd,Cc,YZaw),(Xd,Cc,YZax),(Xd,Cc,YZay),(Xd,Cc,YZaz),(Xd,Cc,YZba), (Xd,Cc,YZbb),(Xd,Cc,YZbc),(Xd,Cc,YZbd),(Xd,Cc,YZbe),(Xd,Cc,YZbf),(Xd,Cc,YZbg), (Xd,Cc,YZbh),(Xd,Cc,YZbi),(Xd,Cc,YZbj),(Xd,Cc,YZbk),(Xd,Cc,YZbl),(Xd,Cc, YZbm),(Xd,Cc,YZbn),(Xd,Cc,YZbo),(Xd,Cc,YZbp),(Xd,Cc,YZbq),(Xd,Cc,YZbr),(Xd, Cc,YZbs),(Xd,Cc,YZbt),(Xd,Cc,YZbu),(Xd,Cc,YZbv),(Xd,Cc,YZbw),(Xd,Cc,YZbx), (Xd,Cc,YZby),(Xd,Cc,YZbz),(Xd,Cc,YZca),(Xd,Cc,YZcb),(Xd,Cc,YZcc),(Xd,Cc,YZdc), (Xd,Cc,YZee),(Xd,Cc,YZcf),(Xd,Cc,YZcg),(Xd,Cc,YZch),(Xd,Cc,YZci),(Xd,Cc, YZcj),(Xd,Cc,YZck),(Xd,Cc,YZcl),(Xd,Cc,YZcm),(Xd,Cc,YZcn),(Xd,Cc,YZco),(Xd, Cc,YZcp),(Xd,Cc,YZcq),(Xd,Cc,YZcr),(Xd,Cc,YZes),(Xd,Cc,YZct),(Xd,Cc,YZcu),(Xd, Cc,YZcv),(Xd,Cc,YZcw),(Xd,Cc,YZcx),(Xd,Cc,YZcy),(Xd,Cc,YZcz),(Xd,Cc,YZda), (Xd,Cc,YZdb),(Xd,Cc,YZdc),(Xd,Cc,YZdd),(Xd,Cc,YZde),(Xd,Cc,YZdf),(Xd,Cc,YZdg), (Xd,Cc,YZdh),(Xd,Cc,YZdi),(Xd,Cc,YZdj),(Xd,Cc,YZdk),(Xd,Cc,YZdl),(Xd,Cc, YZdm),(Xd,Cc,YZdn),(Xd,Cc,YZdo),(Xd,Cc,YZdp),(Xd,Cc,YZdq),(Xd,Cc,YZdr),(Xd, Cc,YZds),(Xd,Cc,Yzdt),(Xd,Cc,YZdu),(Xd,Cc,YZdv),(Xd,Cc,YZdw),(Xd,Cc,YZdx), (Xd,Cc,YZdy),(Xd,Cc,YZdz),(Xd,Cc,YZea),(Xd,Cc,YZeb),(Xd,Cc,YZec),(Xd,Cc,YZed), (Xd,Cc,YZee),(Xd,Cc,YZef),(Xd,Cc,YZeg),(Xd,Cc,YZeh),(Xd,Cc,YZei),(Xd,Cc, YZej)((XdCcc,YZek),(XdCcc,YZel),(XdCcc,YZem),XxdCcc,YZen),(XdCcc,YZeo),(Xd, Cc,YZep),(XdCc,rYZeq),(Xd,Cc,YZer),(Xd,Cc,YZes),(Xd,Cc,YZet),(Xd,Cc,YZeu),(Xd, Cc,YZev),(Xd,Cc,YZew),(Xd,Cc,YZex),(Xd,Cc,YZey),(Xd,Cc,YZez),(Xd,Cc,YZfa), (Xd,Cc,YZfb),(Xd,Cc,YZfc),(Xd,Cc,YZfd),(Xd,Cc,YZfe),(Xd,Cc,YZff),(Xd,Cc,YZfg), (Xd,Cc,YZfh),(Xd,Cc,YZfi),(Xd,Cc,YZj),(Xd,Cc,YZfk),(Xd,Cc,YZfl),(Xd,Cc,YZfm), (Xd,Cc,YZfn),(Xd,Cc,YZfo),(Xd,Cc,YZfp),(Xd,Cc,YZfq),(Xd,Cc,YZfr),(Xd,Cc,YZfs), (Xd,Cc,YZft),(Xd,Cc,YZfu),(Xd,Cc,YZfv),(Xd,Cc,YZfw),(Xd,Cc,YZfx),(Xd,Cc, YZfy),(Xd,Cc,YZfz),(Xd,Cc,YZga),(Xd,Cc,YZgb),(Xd,Cc,YZgc),(Xd,Cc,YZgd),(Xd, Cc,YZge),(Xd,Cc,YZgf),(Xd,Cc,YZgg),(Xd,Cc,YZgh),(Xd,Cc,YZgi),(Xd,Cc,YZgj),(Xd, Cc,YZgk),(Xd,Cc,YZgl),(Xd,Cc,YZgm),(Xd,Cc,YZgn),(Xd,Cc,YZgo),(Xd,Cc,YZgp), (Xd,Cc,YZgq),(Xd,Cc,YZgr),(Xd,Cc,YZgs),(Xd,Cc,YZgt),(Xd,Cd,YZa),(Xd,Cd,YZb), (Xd,Cd,YZc),(Xd,Cd,YZd),(Xd,Cd,YZe),(Xd,Cd,YZf),(Xd,Cd,YZg),(Xd,Cd,YZh), (Xd,Cd,YZi),(Xd,Cd,YZj),(Xd,Cd,YZk),(Xd,Cd,YZl),(Xd,Cd,YZm),(Xd,Cd,YZn),(Xd, Cd,YZo),(Xd,Cd,YZp),(Xd,Cd,YZq),(Xd,Cd,YZr),(Xd,Cd,YZs),(Xd,Cd,YZt),(Xd,Cd, YZu),(Xd,Cd,YZv),(Xd,Cd,YZw),(Xd,Cd,YZx),(Xd,Cd,YZy),(Xd,Cd,YZz),(Xd,Cd, YZaa),(Xd,Cd,YZab),(Xd,Cd,YZac),(Xd,Cd,YZad),(Xd,Cd,YZae),(Xd,Cd,YZaf),(Xd, Cd,YZag),(Xd,Cd,YZah),(Xd,Cd,YZai),(Xd,Cd,YZaj),(Xd,Cd,YZak),(Xd,Cd,YZal),(Xd, Cd,YZam),(Xd,Cd,YZan),(Xd,Cd,YZao),(Xd,Cd,YZap),(Xd,Cd,YZaq),(Xd,Cd,YZar), (Xd,Cd,YZas),(Xd,Cd,YZat),(Xd,Cd,YZau),(Xd,Cd,YZav),(Xd,Cd,YZaw),(Xd,Cd, YZax),(Xd,Cd,YZay),(Xd,Cd,YZaz),(Xd,Cd,YZba),(Xd,Cd,YZbb),(Xd,Cd,YZbc),(Xd, Cd,YZbd),(Xd,Cd,YZbe),(Xd,Cd,YZbf),(Xd,Cd,YZbg),(Xd,Cd,YZbh),(Xd,Cd,YZbi), (Xd,Cd,YZbj),(Xd,Cd,YZbk),(Xd,Cd,YZbl),(Xd,Cd,YZbm),(Xd,Cd,YZbn),(Xd,Cd,YZbo), (Xd,Cd,YZbp),(Xd,Cd,YZbq),(Xd,Cd,YZbr),(Xd,Cd,YZbs),(Xd,Cd,YZbt),(Xd,Cd, YZbu),(Xd,Cd,YZbv),(Xd,Cd,YZbw),(Xd,Cd,YZbx),(Xd,Cd,YZby),(Xd,Cd,YZbz),(Xd, Cd,YZea),(Xd,Cd,YZcb),(Xd,Cd,YZcc),(Xd,Cd,YZcd),(Xd,Cd,YZce),(Xd,Cd,YZcf), (Xd,Cd,YZcg),(Xd,Cd,YZch),(Xd,Cd,YZci),(Xd,Cd,YZcj),(Xd,Cd,YZck),(Xd,Cd,YZcl), (Xd,Cd,YZcm),(Xd,Cd,YZen),(Xd,Cd,YZco),(Xd,Cd,YZcp),(Xd,Cd,YZcq),(Xd, Cd,YZcr),(Xd,Cd,YZcs),(Xd,Cd,YZct),(Xd,Cd,YZcu),(Xd,Cd,YZcv),(Xd,Cd,YZcw),(Xd, Cd,YZcx),(Xd,Cd,YZcy),(Xd,Cd,YZcz),(Xd,Cd,YZda),(Xd,Cd,YZdb),(Xd,Cd,YZdc), (Xd,Cd,YZdd),(Xd,Cd,YZde),(Xd,Cd,YZdf),(Xd,Cd,YZdg),(Xd,Cd,YZdh),(Xd,Cd, YZdi),(Xd,Cd,YZdj),(Xd,Cd,YZdk),(Xd,Cd,YZdl),(Xd,Cd,YZdm),(Xd,Cd,YZdn),(Xd, Cd,YZdo),(Xd,Cd,YZdp),(Xd,Cd,YZdq),(Xd,Cd,YZdr),(Xd,Cd,YZds),(Xd,Cd,Yzdt), (Xd,Cd,YZdu),(Xd,Cd,YZdv),(Xd,Cd,YZdw),(Xd,Cd,YZdx),(Xd,Cd,YZdy),(Xd,Cd, YZdz),(Xd,Cd,YZea),(Xd,Cd,YZeb),(Xd,Cd,YZec),(Xd,Cd,YZed),(Xd,Cd,YZee),(Xd, Cd,YZef),(Xd,Cd,YZeg),(Xd,Cd,YZeh),(Xd,Cd,YZei),(Xd,Cd,YZej),(Xd,Cd,YZek),(Xd, Cd,YZel),(Xd,Cd,YZem),(Xd,Cd,YZen),(Xd,Cd,YZeo),(Xd,Cd,YZep),(Xd,Cd,YZeq), (Xd,Cd,YZer),(Xd,Cd,YZes),(Xd,Cd,YZet),(Xd,Cd,YZeu),(Xd,Cd,YZev),(Xd,Cd, YZew),(Xd,Cd,YZex),(Xd,Cd,YZey),(Xd,Cd,YZez),(Xd,Cd,YZfa),(Xd,Cd,YZfb),(Xd, Cd,YZfc),(Xd,Cd,YZfd),(Xd,Cd,YZfe),(Xd,Cd,YZff),(Xd,Cd,YZfg),(Xd,Cd,YZfh),(Xd, Cd,YZfi),(Xd,Cd,YZfj),(Xd,Cd,YZfk),(Xd,Cd,YZfl),(Xd,Cd,YZfm),(Xd,Cd,YZfn),(Xd, Cd,YZfo),(Xd,Cd,YZfp),(Xd,Cd,YZfq),(Xd,Cd,YZfr),(Xd,Cd,YZfs),(Xd,Cd,YZft), (Xd,Cd,YZfu),(Xd,Cd,YZfv),(Xd,Cd,YZfw),(Xd,Cd,YZfx),(Xd,Cd,YZfy),(Xd,Cd,YZfz), (Xd,Cd,YZga),(Xd,Cd,YZgb),(Xd,Cd,YZgc),(Xd,Cd,YZgd),(Xd,Cd,YZge),(Xd,Cd, YZgf),(Xd,Cd,YZgg),(Xd,Cd,YZgh),(Xd,Cd,YZgi),(Xd,Cd,YZgj),(Xd,Cd,YZgk),(Xd, Cd,YZgl),(Xd,Cd,YZgm),(Xd,Cd,YZgn),(Xd,Cd,YZgo),(Xd,Cd,YZgp),(Xd,Cd,YZgq), (Xd,Cd,YZgr),(Xd,Cd,YZgs),(Xd,Cd,YZgt),(Xd,Ce,YZa),(Xd,Ce,YZb),(Xd,Ce,YZc), (Xd,Ce,YZd),(Xd,Ce,YZe),(Xd,Ce,YZf),(Xd,Ce,YZg),(Xd,Ce,YZh),(Xd,Ce,YZi),(Xd, Ce,YZj),(Xd,Ce,YZk),(Xd,Ce,YZl),(Xd,Ce,YZm),(Xd,Ce,YZn),(Xd,Ce,YZo),(Xd,Ce, YZp),(Xd,Ce,YZq),(Xd,Ce,YZr),(Xd,Ce,YZs),(Xd,Ce,YZt),(Xd,Ce,YZu),(Xd,Ce,YZv), (Xd,Ce,YZw),(Xd,Ce,YZx),(Xd,Ce,YZy),(Xd,Ce,YZz),(Xd,Ce,YZaa),(Xd,Ce,YZab), (Xd,Ce,YZac),(Xd,Ce,YZad),(Xd,Ce,YZae),(Xd,Ce,YZaf),(Xd,Ce,YZag),(Xd,Ce,YZah), (Xd,Ce,YZai),(Xd,Ce,YZaj),(Xd,Ce,YZak),(Xd,Ce,YZal),(Xd,Ce,YZam),(Xd,Ce, YZan),(Xd,Ce,YZao),(Xd,Ce,YZap),(Xd,Ce,YZaq),(Xd,Ce,YZar),(Xd,Ce,YZas),(Xd, Ce,YZat),(Xd,Ce,YZau),(Xd,Ce,YZav),(Xd,Ce,YZaw),(Xd,Ce,YZax),(Xd,Ce,YZay),(Xd, Ce,YZaz),(Xd,Ce,YZba),(Xd,Ce,YZbb),(Xd,Ce,YZbc),(Xd,Ce,Y Zbd),(Xd,Ce,YZbe), (Xd,Ce,YZbf),(Xd,Ce,YZbg),(Xd,Ce,YZbh),(Xd,Ce,YZbi),(Xd,Ce,YZbj),(Xd,Ce,YZbk), (Xd,Ce,YZbl),(Xd,Ce,YZbm),(Xd,Ce,YZbn),(Xd,Ce,YZbo),(Xd,Ce,YZbp),(Xd, Ce,YZbq),(Xd,Ce,YZbr),(Xd,Ce,YZbs),(Xd,Ce,YZbt),(Xd,Ce,YZbu),(Xd,Ce,YZbv),(Xd, Ce,YZbw),(Xd,Ce,YZbx),(Xd,Ce,YZby),(Xd,Ce,YZbz),(Xd,Ce,YZca),(Xd,Ce,YZcb), (Xd,Ce,YZcc),(Xd,Ce,YZcd),(Xd,Ce,YZce),(Xd,Ce,YZcf),(Xd,Ce,YZcg),(Xd,Ce, YZch),(Xd,Ce,YZci),(Xd,Ce,YZcj),(Xd,Ce,YZek),(Xd,Ce,YZcl),(Xd,Ce,YZcm),(Xd, Ce,YZcn),(Xd,Ce,YZco),(Xd,Ce,YZcp),(Xd,Ce,YZcq),(Xd,Ce,YZer),(Xd,Ce,YZcs),(Xd, Ce,YZct),(Xd,Ce,YZcu),(Xd,Ce,YZcv),(Xd,Ce,YZcw),(Xd,Ce,YZcx),(Xd,Ce,YZcy), (Xd,Ce,YZcz),(Xd,Ce,YZda),(Xd,Ce,YZdb),(Xd,Ce,YZdc),(Xd,Ce,YZdd),(Xd,Ce, YZde),(Xd,Ce,YZdf),(Xd,Ce,YZdg),(Xd,Ce,YZdh),(Xd,Ce,YZdi),(Xd,Ce,YZdj),(Xd, Ce,YZdk),(Xd,Ce,YZdl),(Xd,Ce,YZdm),(Xd,Ce,YZdn),(Xd,Ce,YZdo),(Xd,Ce,YZdp), (Xd,Ce,YZdq),(Xd,Ce,YZdr),(Xd,Ce,YZds),(Xd,Ce,Yzdt),(Xd,Ce,YZdu),(Xd,Ce,YZdv), (Xd,Ce,YZdw),(Xd,Ce,YZdx),(Xd,Ce,YZdy),(Xd,Ce,YZdz),(Xd,Ce,YZea),(Xd,Ce, YZeb),(Xd,Ce,YZec),(Xd,Ce,YZed),(Xd,Ce,YZee),(Xd,Ce,YZef),(Xd,Ce,YZeg),(Xd, Ce,Yeh),(Xd,Ce,YZei),(Xd,Ce,YZej),(Xd,Ce,YZek),(Xd,Ce,YZel),(Xd,Ce,YZem),(Xd, Ce,YZen),(Xd,Ce,YZeo),(Xd,Ce,YZep),(Xd,Ce,YZeq),(Xd,Ce,YZer),(Xd,Ce,YZes), (Xd,Ce,YZet),(Xd,Ce,YZeu),(Xd,Ce,YZev),(Xd,Ce,YZew),(Xd,Ce,YZex),(Xd,Ce,YZey), (Xd,Ce,YZez),(Xd,Ce,YZfa),(Xd,Ce,YZfb),(Xd,Ce,YZfc),(Xd,Ce,YZfd),(Xd,Ce, YZfe),(Xd,Ce,YZff),(Xd,Ce,YZfg),(Xd,Ce,YZfh),(Xd,Ce,YZfi),(Xd,Ce,YZfj),(Xd,Ce, YZfk),(Xd,Ce,YZfl),(Xd,Ce,YZfm),(Xd,Ce,YZfn),(Xd,Ce,YZfo),(Xd,Ce,YZfp),(Xd,Ce, YZfq),(Xd,Ce,YZfr),(Xd,Ce,YZfs),(Xd,Ce,YZft),(Xd,Ce,YZfu),(Xd,Ce,YZfv),(Xd,Ce, YZfw),(Xd,Ce,YZfx),(Xd,Ce,YZfy),(Xd,Ce,YZfz),(Xd,Ce,YZga),(Xd,Ce,YZgb),(Xd, Ce,YZgc),(Xd,Ce,YZgd),(Xd,Ce,YZge),(Xd,Ce,YZgf),(Xd,Ce,YZgg),(Xd,Ce,YZgh), (Xd,Ce,YZgi),(Xd,Ce,YZgj),(Xd,Ce,YZgk),(Xd,Ce,YZgl),(Xd,Ce,YZgm),(Xd,Ce,YZgn), (Xd,Ce,YZgo),(Xd,Ce,YZgp),(Xd,Ce,YZgq),(Xd,Ce,YZgr),(Xd,Ce,YZgs),(Xd,Ce, YZgt),(Xd,Cf,YZa),(Xd,Cf,YZb),(Xd,Cf,YZc),(Xd,Cf,YZd),(Xd,Cf,YZe),(Xd,Cf,YZf), (Xd,Cf,YZg),(Xd,Cf,YZh),(Xd,Cf,YZi),(Xd,Cf,YZj),(Xd,Cf,YZk),(Xd,Cf,YZl),(Xd,Cf, YZm),(Xd,Cf,YZn),(Xd,Cf,YZo),(Xd,Cf,YZp),(Xd,Cf,YZq),(Xd,Cf,YZr),(Xd,Cf,YZs), (Xd,Cf,YZt),(Xd,Cf,YZu),(Xd,Cf,YZv),(Xd,Cf,YZw),(Xd,Cf,YZx),(Xd,Cf,YZy),(Xd, Cf,YZz),(Xd,Cf,YZaa),(Xd,Cf,YZab),(Xd,Cf,YZac),(Xd,Cf,YZad),(Xd,Cf,YZae),(Xd, Cf,YZaf),(Xd,Cf,YZag),(Xd,Cf,YZah),(Xd,Cf,YZai),(Xd,Cf,YZaj),(Xd,Cf,YZak),(Xd, Cf,YZal),(Xd,Cf,YZam),(Xd,Cf,YZan),(Xd,Cf,YZao),(Xd,Cf,YZap),(Xd,Cf,YZaq),(Xd, Cf,YZar),(Xd,Cf,YZas),(Xd,Cf,YZat),(Xd,Cf,YZau),(Xd,Cf,YZav),(Xd,Cf,YZaw), (Xd,Cf,YZax),(Xd,Cf,YZay),(Xd,Cf,YZaz),(Xd,Cf,YZba),(Xd,Cf,YZbb),(Xd,Cf,YZbc), (Xd,Cf,YZbd),(Xd,Cf,YZbe),(Xd,Cf,YZbf),(Xd,Cf,YZbg),(Xd,Cf,YZbh),(Xd,Cf,YZbi), (Xd,Cf,YZbj),(Xd,Cf,YZbk),(Xd,Cf,YZbl),(Xd,Cf,YZbm),(Xd,Cf,YZbn),(Xd,Cf,YZbo), (Xd,Cf,YZbp),(Xd,Cf,YZbq),(Xd,Cf,YZbr),(Xd,Cf,YZbs),(Xd,Cf,YZbt),(Xd,Cf, YZbu),(Xd,Cf,YZbv),(Xd,Cf,YZbw),(Xd,Cf,YZbx),(Xd,Cf,YZby),(Xd,Cf,YZbz),(Xd, Cf,YZca),(Xd,Cf,YZcb),(Xd,Cf,YZcc),(Xd,Cf,YZcd),(Xd,Cf,YZce),(Xd,Cf,YZcf),(Xd, Cf,YZcg),(Xd,Cf,YZch),(Xd,Cf,YZci),(Xd,Cf,YZcj),(Xd,Cf,YZck),(Xd,Cf,YZcl),(Xd, Cf,YZcm),(Xd,Cf,YZcn),(Xd,Cf,YZco),(Xd,Cf,YZcp),(Xd,Cf,YZcq),(Xd,Cf,YZcr),(Xd, Cf,YZcs),(Xd,Cf,YZct),(Xd,Cf,YZcu),(Xd,Cf,YZcv),(Xd,Cf,YZcw),(Xd,Cf,YZcx), (Xd,Cf,YZcy),(Xd,Cf,YZcz),(Xd,Cf,YZda),(Xd,Cf,YZdb),(Xd,Cf,YZdc),(Xd,Cf,YZdd), (Xd,Cf,YZde),(Xd,Cf,YZdf),(Xd,Cf,YZdg),(Xd,Cf,YZdh),(Xd,Cf,YZdi),(Xd,Cf,YZdj), (Xd,Cf,YZdk),(Xd,Cf,YZdl),(Xd,Cf,YZdm),(Xd,Cf,YZdn),(Xd,Cf,YZdo),(Xd,Cf, YZdp),(Xd,Cf,YZdq),(Xd,Cf,YZdr),(Xd,Cf,YZds),(Xd,Cf,Yzdt),(Xd,Cf,YZdu),(Xd,Cf, YZdv),(Xd,Cf,YZdw),(Xd,Cf,YZdx),(Xd,Cf,YZdy),(Xd,Cf,YZdz),(Xd,Cf,YZea),(Xd, Cf,YZeb),(Xd,Cf,YZec),(Xd,Cf,YZed),(Xd,Cf,YZee),(Xd,Cf,YZef),(Xd,Cf,YZeg),(Xd, Cf,YZeh),(Xd,Cf,YZei),(Xd,Cf,YZej),(Xd,Cf,YZek),(Xd,Cf,YZel),(Xd,Cf,YZem),(Xd, Cf,YZen),(Xd,Cf,YZeo),(Xd,Cf,YZep),(Xd,Cf,YZeq),(Xd,Cf,YZer),(Xd,Cf,YZes),(Xd, Cf,YZet),(Xd,Cf,Yeu),(Xd,Cf,YZev),(Xd,Cf,YZew),(Xd,Cf,YZex),(Xd,Cf,YZey), (Xd,Cf,YZez),(Xd,Cf,YZfa),(Xd,Cf,YZfb),(Xd,Cf,YZfc),(Xd,Cf,YZfd),(Xd,Cf,YZfe), (Xd,Cf,YZff),(Xd,Cf,YZfg),(Xd,Cf,YZfh),(Xd,Cf,YZfi),(Xd,Cf,YZfj),(Xd,Cf,YZfk),(Xd, Cf,YZfl),(Xd,Cf,YZfm),(Xd,Cf,YZfn),(Xd,Cf,YZfo),(Xd,Cf,YZfp),(Xd,Cf,YZfq),(Xd, Cf,YZfr),(Xd,Cf,YZfs),(Xd,Cf,YZft),(Xd,Cf,YZfu),(Xd,Cf,YZfv),(Xd,Cf,YZfw),(Xd, Cf,YZfx),(Xd,Cf,YZfy),(Xd,Cf,YZfz),(Xd,Cf,YZga),(Xd,Cf,YZgb),(Xd,Cf,YZgc), (Xd,Cf,YZgd),(Xd,Cf,YZge),(Xd,Cf,YZgf),(Xd,Cf,YZgg),(Xd,Cf,YZgh),(Xd,Cf,YZgi), (Xd,Cf,YZgj),(Xd,Cf,YZgk),(Xd,Cf,YZgl),(Xd,Cf,YZgm),(Xd,Cf,YZgn),(Xd,Cf,YZgo), (Xd,Cf,YZgp),(Xd,Cf,YZgq),(Xd,Cf,YZgr),(Xd,Cf,YZgs),(Xd,Cf,YZgt), (Xe,Ca,YZa),(Xe,Ca,YZb),(Xe,Ca,YZc),(Xe,Ca,YZd),(Xe,Ca,YZe),(Xe,Ca,YZf),(Xe, Ca,YZg),(Xe,Ca,YZh),(Xe,Ca,YZi),(Xe,Ca,YZj),(Xe,Ca,YZk),(Xe,Ca,YZl),(Xe,Ca,YZm), (Xe,Ca,YZn),(Xe,Ca,YZo),(Xe,Ca,YZp),(Xe,Ca,YZq),(Xe,Ca,YZr),(Xe,Ca,YZs), (Xe,Ca,YZt),(Xe,Ca,YZu),(Xe,Ca,YZv),(Xe,Ca,YZw),(Xe,Ca,YZx),(Xe,Ca,YZy),(Xe, Ca,YZz),(Xe,Ca,YZaa),(Xe,Ca,YZab),(Xe,Ca,YZac),(Xe,Ca,YZad),(Xe,Ca,YZae),(Xe, Ca,YZaf),(Xe,Ca,YZag),(Xe,Ca,YZah),(Xe,Ca,YZai),(Xe,Ca,YZaj),(Xe,Ca,YZak),(Xe, Ca,YZal),(Xe,Ca,YZam),(Xe,Ca,YZan),(Xe,Ca,YZao),(Xe,Ca,YZap),(Xe,Ca,YZaq), (Xe,Ca,YZar),(Xe,Ca,YZas),(Xe,Ca,YZat),(Xe,Ca,YZau),(Xe,Ca,YZav),(Xe,Ca,YZaw), (Xe,Ca,YZax),(Xe,Ca,YZay),(Xe,Ca,YZaz),(Xe,Ca,YZba),(Xe,Ca,YZbb),(Xe,Ca,YZbc), (Xe,Ca,YZbd),(Xe,Ca,YZbe),(Xe,Ca,YZbf),(Xe,Ca,YZbg),(Xe,Ca,YZbh),(Xe,Ca, YZbi),(Xe,Ca,YZbj),(Xe,Ca,YZbk),(Xe,Ca,YZbl),(Xe,Ca,YZbm),(Xe,Ca,YZbn),(Xe, Ca,YZbo),(Xe,Ca,YZbp),(Xe,Ca,YZbq),(Xe,Ca,YZbr),(Xe,Ca,YZbs),(Xe,Ca,YZbt),(Xe, Ca,YZbu),(Xe,Ca,YZbv),(Xe,Ca,YZbw),(Xe,Ca,YZbx),(Xe,Ca,YZby),(Xe,Ca,YZbz), (Xe,Ca,YZca),(Xe,Ca,YZcb),(Xe,Ca,YZcc),(Xe,Ca,YZcd),(Xe,Ca,YZce),(Xe,Ca,YZcf), (Xe,Ca,YZcg),(Xe,Ca,YZch),(Xe,Ca,YZci),(Xe,Ca,YZcj),(Xe,Ca,YZek),(Xe,Ca,YZel), (Xe,Ca,YZcm),(Xe,Ca,YZcn),(Xe,Ca,YZco),(Xe,Ca,YZcp),(Xe,Ca,YZcq),(Xe,Ca, YZcr),(Xe,Ca,YZcs),(Xe,Ca,YZct),(Xe,Ca,YZcu),(Xe,C a,YZcv),(Xe,Ca,YZcw),(Xe, Ca,YZcx),(Xe,Ca,YZcy),(Xe,Ca,YZcz),(Xe,Ca,YZda),(Xe,Ca,YZdb),(Xe,Ca,YZdc),(Xe, Ca,YZdd),(Xe,Ca,YZde),(Xe,Ca,YZdf),(Xe,Ca,YZdg),(Xe,Ca,YZdh),(Xe,Ca,YZdi), (Xe,Ca,YZdj),(Xe,Ca,YZdk),(Xe,Ca,YZdl),(Xe,Ca,YZdm),(Xe,Ca,YZdn),(Xe,Ca,YZdo), (Xe,Ca,YZdp),(Xe,Ca,YZdq),(Xe,Ca,YZdr),(Xe,Ca,YZds),(Xe,Ca,Yzdt),(Xe,Ca,YZdu), (Xe,Ca,YZdv),(Xe,Ca,YZdw),(Xe,Ca,YZdx),(Xe,Ca,YZdy),(Xe,Ca,YZdz),(Xe,Ca, YZea),(Xe,Ca,YZeb),(Xe,Ca,YZec),(Xe,Ca,YZed),(Xe,Ca,YZee),(Xe,Ca,YZef),(Xe, Ca,YZeg),(Xe,Ca,YZeh),(Xe,Ca,YZei),(XeCa,aYZej),(Xe,Ca,YZek),(Xe,Ca,YZel),(Xe, Ca,YZem),(Xe,Ca,YZen),(Xe,Ca,YZeo),(Xe,Ca,YZep),(Xe,Ca,YZeq),(Xe,Ca,YZer),(Xe, Ca,YZes),(Xe,Ca,YZet),(Xe,Ca,YZeu),(Xe,Ca,YZev),(Xe,Ca,YZew),(Xe,Ca,YZex), (Xe,Ca,YZey),(Xe,Ca,YZez),(Xe,Ca,YZfa),(Xe,Ca,YZfb),(Xe,Ca,YZfc),(Xe,Ca,YZfd), (Xe,Ca,YZfe),(Xe,Ca,YZff),(Xe,Ca,YZfg),(Xe,Ca,YZfh),(Xe,Ca,YZfi),(Xe,Ca,YZfj), (Xe,Ca,YZfk),(Xe,Ca,YZfl),(Xe,Ca,YZfm),(Xe,Ca,YZfn),(Xe,Ca,YZfo),(Xe,Ca,YZfp), (Xe,Ca,YZfq),(Xe,Ca,YZfr),(Xe,Ca,YZfs),(Xe,Ca,YZft),(Xe,Ca,YZfu),(Xe,Ca,YZfv), (Xe,Ca,YZfwv),(Xe,Ca,YZfx),(Xe,Ca,YZfy),(Xe,Ca,YZfz),(Xe,Ca,YZga),(Xe,Ca,YZgb), (Xe,Ca,YZgc),(Xe,Ca,YZgd),(Xe,Ca,YZge),(Xe,Ca,YZgf),(Xe,Ca,YZgg),(Xe,Ca, YZgh),(Xe,Ca,YZgi),(Xe,Ca,YZgj),(Xe,Ca,YZgk),(Xe,Ca,YZgl),(Xe,Ca,YZgm),(Xe, Ca,YZgn),(Xe,Ca,YZgo),(Xe,Ca,YZgp),(Xe,Ca,YZgq),(Xe,Ca,YZgr),(Xe,Ca,YZgs),(Xe, Ca,YZgt),(Xe,Cb,YZa),(Xe,Cb,YZb),(Xe,Cb,YZc),(Xe,Cb,YZd),(Xe,Cb,YZe),(Xe, Cb,YZf),(Xe,Cb,YZg),(Xe,Cb,YZh),(Xe,Cb,YZi),(Xe,Cb,YZj),(Xe,Cb,YZk),(Xe,Cb, YZl),(Xe,Cb,YZm),(Xe,Cb,YZn),(Xe,Cb,YZo),(Xe,Cb,YZp),(Xe,Cb,YZq),(Xe,Cb,YZr), (Xe,Cb,YZs),(Xe,Cb,YZt),(Xe,Cb,YZu),(Xe,Cb,YZv),(Xe,Cb,YZw),(Xe,Cb,YZx),(Xe, Cb,YZy),(Xe,Cb,YZz),(Xe,Cb,YZaa),(Xe,Cb,YZab),(Xe,Cb,YZac),(Xe,Cb,YZad), (Xe,Cb,YZae),(Xe,Cb,YZaf),(Xe,Cb,YZag),(Xe,Cb,YZah),(Xe,Cb,YZai),(Xe,Cb,YZaj), (Xe,Cb,YZak),(Xe,Cb,YZal),(Xe,Cb,YZam),(Xe,Cb,YZan),(Xe,Cb,YZao),(Xe,Cb,YZap), (Xe,Cb,YZaq),(Xe,Cb,YZar),(Xe,Cb,YZas),(Xe,Cb,YZat),(Xe,Cb,YZau),(Xe,Cb, YZav),(Xc,Cb,YZaw),(Xe,Cb,YZax),(Xe,Cb,YZay),(Xe,Cb,YZaz),(Xe,Cb,YZba),(Xe, Cb,YZbb),(Xe,Cb,YZbc),(Xe,Cb,YZbd),(Xe,Cb,YZbe),(Xe,Cb,YZbf),(Xe,Cb,YZbg),(Xe, Cb,YZbh),(Xe,Cb,YZbi),(Xe,Cb,YZbj),(Xe,Cb,YZbk),(Xe,Cb,YZbl),(Xe,Cb,YZbm), (Xe,Cb,YZbn),(Xe,Cb,YZbo),(Xe,Cb,YZbp),(Xe,Cb,YZbq),(Xe,Cb,YZbr),(Xe,Cb, YZbs),(Xe,Cb,YZbt),(Xe,Cb,YZbu),(Xe,Cb,YZbv),(Xe,Cb,YZbw),(Xe,Cb,YZbx),(Xe, Cb,YZby),(Xe,Cb,YZbz),(Xe,Cb,YZca),(Xe,Cb,YZcb),(Xe,Cb,YZcc),(Xe,Cb,YZcd),(Xe, Cb,YZce),(Xe,Cb,YZcf),(Xe,Cb,YZcg),(Xe,Cb,YZch),(Xe,Cb,YZci),(Xe,Cb,YZcj), (Xe,Cb,YZck),(Xe,Cb,YZcl),(Xe,Cb,YZcm),(Xe,Cb,YZcn),(Xe,Cb,YZco),(Xe,Cb,YZcp), (Xe,Cb,YZcq),(Xe,Cb,YZcr),(Xe,Cb,YZcs),(Xe,Cb,YZet),(Xe,Cb,YZcu),(Xe,Cb, YZcv),(Xe,Cb,YZcw),(Xe,Cb,YZcx),(Xe,Cb,YZcy),(Xe,Cb,YZcz),(XO,Cb,YZda),(Xe, Cb,YZdb),(Xe,Cb,YZdc),(Xe,Cb,YZdd),(Xe,Cb,YZde),(Xe,Cb,YZdf),(Xe,Cb,YZdg),(Xe, Cb,YZdh),(Xe,Cb,YZdi),(Xe,Cb,YZdj),(Xe,Cb,YZdk),(Xe,Cb,YZdl),(Xe,Cb,YZdm), (Xe,Cb,YZdn),(Xe,Cb,YZdo),(Xe,Cb,YZdp),(Xe,Cb,YZdq),(Xe,Cb,YZdr),(Xe,Cb, YZds),(Xe,Cb,Yzdt),(Xe,Cb,YZdu),(Xe,Cb,YZdv),(Xe,Cb,YZdw),(Xe,Cb,YZdx),(Xe, Cb,YZdy),(Xe,Cb,YZdz),(Xe,Cb,YZea),(Xe,Cb,YZeb),(Xe,Cb,YZec),(Xe,Cb,YZed),(Xe, Cb,YZee),(Xe,Cb,YZef),(Xe,Cb,YZeg),(Xe,Cb,YZeh),(Xe,Cb,YZei),(Xe,Cb,YZej), (Xe,Cb,YZek),(Xe,Cb,YZel),(Xe,Cb,YZem),(Xe,Cb,YZen),(Xe,Cb,YZeo),(Xe,Cb,YZep), (Xe,Cb,YZeq),(Xe,Cb,YZer),(Xe,Cb,YZes),(Xe,Cb,YZet),(Xe,Cb,YZeu),(Xe,Cb, YZev),(Xe,Cb,YZew),(Xe,Cb,YZex),(Xe,Cb,YZey),(Xe,Cb,YZez),(Xe,Cb,YZfa),(Xe, Cb,YZfb),(Xe,Cb,YZfc),(Xe,Cb,YZfd),(Xe,Cb,YZfe),(Xe,Cb,YZff),(Xe,Cb,YZfg),(Xe, Cb,YZfh),(Xe,Cb,YZfi),(Xe,Cb,YZfj),(Xe,Cb,YZfk),(Xe,Cb,YZfl),(Xe,Cb,YZfm),(Xe, Cb,YZfn),(Xe,Cb,YZfo),(Xe,Cb,YZfp),(Xe,Cb,YZfq),(Xe,Cb,YZfr),(Xe,Cb,YZfs),(Xe, Cb,YZft),(Xe,Cb,YZfu),(Xe,Cb,YZfv),(Xe,Cb,YZfwv),(Xe,Cb,YZfx),(Xe,Cb,YZfy), (Xe,Cb,YZfz),(Xe,Cb,YZga),(Xe,Cb,YZgb),(Xe,Cb,YZgc),(Xe,Cb,YZgd),(Xe,Cb,YZge), (Xe,Cb,YZgf),(Xe,Cb,YZgg),(Xe,Cb,YZgh),(Xe,Cb,YZgi),(Xe,Cb,YZgj),(Xe,Cb, YZgk),(Xe,Cb,YZgl),(Xe,Cb,YZgm),(Xe,Cb,YZgn),(Xe,Cb,YZgo),(Xe,Cb,YZgp),(Xe, Cb,YZgq),(Xe,Cb,YZgr),(Xe,Cb,YZgs),(Xe,Cb,YZgt),(Xe,Cc,YZa),(Xe,Cc,YZb),(Xe, Cc,YZc),(Xe,Cc,YZd),(Xe,Cc,YZe),(Xe,Cc,YZf),(Xe,Cc,YZg),(Xe,Cc,YZh),(Xe,Cc, YZi),(Xe,Cc,YZj),(Xe,Cc,YZk),(Xe,Cc,YZl),(Xe,Cc,YZm),(Xe,Cc,YZn),(Xe,Cc,YZo), (Xe,Cc,YZp),(Xe,Cc,YZq),(Xe,Cc,YZr),(Xe,Cc,YZs),(Xe,Cc,YZt),(Xe,Cc,YZu),(Xe, Cc,YZv),(Xe,Cc,YZw),(Xe,Cc,YZx),(Xe,Cc,YZy),(Xe,Cc,YZz),(Xe,Cc,YZaa),(Xe,Cc, YZab),(Xe,Cc,YZac),(Xe,Cc,YZad),(Xe,Cc,YZae),(Xe,Cc,YZaf),(Xe,Cc,YZag),(Xe, Cc,YZah),(Xe,Cc,YZai),(Xe,Cc,YZaj),(Xe,Cc,YZak),(Xe,Cc,YZal),(Xe,Cc,YZam),(Xe, Cc,YZan),(Xe,Cc,YZao),(Xe,Cc,YZap),(Xe,Cc,YZaq),(Xe,Cc,YZar),(Xe,Cc,YZas),(Xe, Cc,YZat),(Xe,Cc,YZau),(Xe,Cc,YZav),(Xe,Cc,YZaw),(Xe,Cc,YZax),(Xe,Cc,YZay), (Xe,Cc,YZaz),(Xe,Cc,YZba),(Xe,Cc,YZbb),(Xe,Cc,YZbc),(Xe,Cc,YZbd),(Xe,Cc,YZbe), (Xe,Cc,YZbf),(Xe,Cc,YZbg),(Xe,Cc,YZbh),(Xe,Cc,YZbi),(Xe,Cc,YZbj),(Xe,Cc,YZbk), (Xe,Cc,YZbl),(Xe,Cc,YZbm),(Xe,Cc,YZbn),(Xe,Cc,YZbo),(Xe,Cc,YZbp),(Xe,Cc, YZbq),(Xe,Cc,YZbr),(Xe,Cc,YZbs),(Xe,Cc,YZbt),(Xe,Cc,YZbu),(Xe,Cc,YZbv),(Xe, Cc,YZbw),(Xe,Cc,YZbx),(Xe,Cc,YZby),(Xe,Cc,YZbz),(Xe,Cc,YZca),(Xe,Cc,YZcb),(Xe, Cc,YZcc),(Xe,Cc,YZed),(Xe,Cc,YZce),(Xe,Cc,YZcf),(Xe,Cc,YZcg),(Xe,Cc,YZch), (Xe,Cc,YZci),(Xe,Cc,YZcj),(Xe,Cc,YZck),(Xe,Cc,YZcl),(Xe,Cc,YZcm),(Xe,Cc,YZcn), (Xe,Cc,YZco),(Xe,Cc,YZcp),(Xe,Cc,YZcq),(Xe,Cc,YZcr),(Xe,Cc,YZcs),(Xe,Cc,YZct), (Xe,Cc,YZcu),(Xe,Cc,YZcv),(Xe,Cc,YZcw),(Xe,Cc,YZcx),(Xe,Cc,YZcy),(Xe,Cc, YZcz),(Xe,Cc,YZda),(Xe,Cc,YZdb),(Xe,Cc,YZdc),(Xe,Cc,YZdd),(Xe,Cc,YZde),(Xe, Cc,YZdf),(Xe,Cc,YZdg),(Xe,Cc,YZdh),(Xe,Cc,YZdi),(Xe,Cc,YZdj),(Xe,Cc,YZdk),(Xd, Cc,YZdl),(Xc,Cc,YZdm),(Xe,Cc,YZdn),(Xe,Cc,YZdo),(Xe,Cc,YZdp),(Xe,Cc,YZdq), (Xe,Cc,YZdr),(Xe,Cc,YZds),(Xe,Cc,Yzdt),(Xe,Cc,YZdu),(Xe,Cc,YZdv),(Xe,Cc,YZdw), (Xe,Cc,YZdx),(Xe,Cc,YZdy),(Xe,Cc,YZdz),(Xe,Cc,YZea),(Xe,Cc,YZeb),(Xe,Cc, YZec),(Xe,Cc,YZed),(Xe,Cc,YZee),(Xe,Cc,YZef),(Xe,Cc,YZeg),(Xe,Cc,YZeh),(Xe,Cc, YZei),(Xe,Cc,YZej),(Xe,Cc,YZek),(Xe,Cc,YZel),(Xe,Cc,YZem),(Xe,Cc,YZen),(Xe, Cc,YZeo),(Xe,Cc,YZep),(Xe,Cc,YZeq),(Xe,Cc,YZer),(Xe,Cc,YZes),(Xe,Cc,YZet),(Xe, Cc,YZeu),(Xe,Cc,YZev),(Xe,Cc,YZew),(Xe,Cc,YZex),(Xe,Cc,YZey),(Xe,Cc,YZez), (Xe,Cc,YZfa),(Xe,Cc,YZfb),(Xe,Cc,YZfc),(Xe,Cc,YZfd),(Xe,Cc,YZfe),(Xe,Cc,YZff), (Xe,Cc,YZfg),(Xe,Cc,YZfh),(Xe,Cc,YZfi),(Xe,Cc,YZfj),(Xe,Cc,YZfk),(Xe,Cc,YZfl),(Xe, Cc,YZfm),(Xe,Cc,YZfn),(Xe,Cc,YZfo),(Xe,Cc,YZfp),(Xe,Cc,YZfq),(Xe,Cc,YZfr), (Xe,Cc,YZfs),(Xe,Cc,YZft),(Xe,Cc,YZfu),(Xe,Cc,YZfv),(Xe,Cc,Yfw),(Xe,Cc,YZfx), (Xe,Cc,YZfy),(Xe,Cc,YZfz),(Xe,Cc,YZga),(Xe,Cc,YZgb),(Xe,Cc,YZgc),(Xe,Cc,YZgd), (Xe,Cc,YZge),(Xe,Cc,YZgf),(Xe,Cc,YZgg),(Xe,Cc,YZgh),(Xe,Cc,YZgi),(Xe,Cc,YZgj), (Xe,Cc,YZgk),(Xe,Cc,YZgl),(Xe,Cc,YZgm),(Xe,Cc,YZgn),(Xe,Cc,YZgo),(Xe,Cc, YZgp),(Xe,Cc,YZgq),(Xe,Cc,YZgr),(Xe,Cc,YZgs),(Xe,Cc,YZgt),(Xe,Cd,YZa),(Xe, Cd,YZb),(Xe,Cd,YZc),(Xe,Cd,YZd),(Xe,Cd,YZe),(Xe,Cd,YZf),(Xe,Cd,YZg),(Xe,Cd, YZh),(Xe,Cd,YZi),(Xe,Cd,YZj),(Xe,Cd,YZk),(Xe,Cd,YZl),(Xe,Cd,YZm),(Xe,Cd,YZn), (Xe,Cd,YZo),(Xe,Cd,YZp),(Xe,Cd,YZq),(Xe,Cd,YZr),(Xe,Cd,YZs),(Xe,Cd,YZt),(Xe, Cd,YZu),(Xe,Cd,YZv),(Xe,Cd,YZw),(Xe,Cd,YZx),(Xe,Cd,YZy),(Xe,Cd,YZz),(Xe, Cd,YZaa),(Xe,Cd,YZab),(Xe,Cd,YZac),(Xe,Cd,YZad),(Xe,Cd,YZae),(Xe,Cd,YZaf),(Xe, Cd,YZag),(Xe,Cd,YZah),(Xe,Cd,YZai),(Xe,Cd,YZaj),(Xe,Cd,YZak),(Xe,Cd,YZal), (Xe,Cd,YZam),(Xe,Cd,YZan),(Xe,Cd,YZao),(Xe,Cd,YZap),(Xe,Cd,YZaq),(Xe,Cd,YZar), (Xe,Cd,YZas),(Xe,Cd,YZat),(Xe,Cd,YZau),(Xe,Cd,YZav),(Xe,Cd,YZaw),(Xe,Cd, YZax),(Xe,Cd,YZay),(Xe,Cd,YZaz),(Xe,Cd,YZba),(Xe,Cd,YZbb),(Xe,Cd,YZbc),(Xe, Cd,YZbd),(Xe,Cd,YZbe),(Xe,Cd,YZbf),(Xe,Cd,YZbg),(Xe,Cd,YZbh),(Xe,Cd,YZbi),(Xe, Cd,YZbj),(Xe,Cd,YZbk),(Xe,Cd,YZbl),(Xe,Cd,YZbm),(Xe,Cd,YZbn),(Xe,Cd,YZbo), (Xe,Cd,YZbp),(Xe,Cd,YZbq),(Xe,Cd,YZbr),(Xe,Cd,YZbs),(Xe,Cd,YZbt),(Xe,Cd,YZbu), (Xe,Cd,YZbv),(Xe,Cd,YZbw),(Xe,Cd,YZbx),(Xe,Cd,YZby),(Xe,Cd,YZbz),(Xe, Cd,YZca),(Xe,Cd,YZcb),(Xe,Cd,YZcc),(Xe,Cd,YZcd),(Xe,Cd,YZce),(Xe,Cd,YZcf),(Xe, Cd,YZcg),(Xe,Cd,YZch),(Xe,Cd,YZci),(Xe,Cd,YZcj),(Xe,Cd,YZck),(Xe,Cd,YZcl), (Xe,Cd,YZcm),(Xe,Cd,YZcn),(Xe,Cd,YZco),(Xe,Cd,YZcp),(Xe,Cd,YZcq),(Xe,Cd,YZcr), (Xe,Cd,YZcs),(Xe,Cd,YZct),(Xe,Cd,YZcu),(Xe,Cd,YZcv),(Xe,Cd,YZcw),(Xe,Cd, YZcx),(Xe,Cd,YZcy),(Xe,Cd,YZcz),(Xe,Cd,YZda),(Xe,Cd,YZdb),(Xe,Cd,YZdc),(Xe, Cd,YZdd),(Xe,Cd,YZde),(Xe,Cd,YZdf),(Xe,Cd,YZdg),(Xe,Cd,YZdh),(Xe,Cd,YZdi),(Xe, Cd,YZdj),(Xe,Cd,YZdk),(Xe,Cd,YZdl),(Xe,Cd,YZdm),(Xe,Cd,YZdn),(Xe,Cd,YZdo), (Xe,Cd,YZdp),(Xe,Cd,YZdq),(Xe,Cd,YZdr),(Xe,Cd,YZds),(Xe,Cd,Yzdt),(Xe,Cd,YZdu), (Xe,Cd,YZdv),(Xe,Cd,YZdw),(Xe,Cd,YZdx),(Xe,Cd,YZdy),(Xe,Cd,YZdz),(Xe, Cd,YZea),(Xe,Cd,YZeb),(Xe,Cd,YZec),(Xe,Cd,YZed),(Xe,Cd,YZee),(Xc,Cd,YZef),(Xe, Cd,YZeg),(Xe,Cd,YZeh),(Xe,Cd,YZei),(Xe,Cd,YZej),Xxe,Cd,YZek),(Xe,Cd,YZel), (Xe,Cd,YZem),(Xe,Cd,YZen),(Xe,Cd,YZeo),(Xe,Cd,YZep),(Xe,Cd,YZeq),(Xe,Cd,YZer), (Xe,Cd,YZes),(Xe,Cd,YZet),(Xe,Cd,YZeu),(Xe,Cd,YZev),(Xe,Cd,YZew),(Xe,Cd, YZex),(Xe,Cd,YZey),(Xe,Cd,YZez),(Xe,Cd,YZfa),(Xe,Cd,YZfb),(Xe,Cd,YZfc),(Xe, Cd,YZfd),(Xe,Cd,YZfe),(Xe,Cd,YZff),(Xe,Cd,YZfg),(Xe,Cd,YZfh),(Xe,Cd,YZfi),(Xe, Cd,YZfj,(Xe,Cd,YZfk),(Xe,Cd,YZfl),(Xe,Cd,YZfm),(Xe,Cd,YZfn),(Xe,Cd,YZfo),(Xe, Cd,YZfp)),(Xe,Cd,YZfq),(Xe,Cd,YZfr),(Xe,Cd,YZfs),(Xe,Cd,YZft),(Xe,Cd,YZfu),(Xe, Cd,YZfv),(Xe,Cd,YZfwr),(Xe,Cd,YZfx),(Xe,Cd,YZfy),(Xe,Cd,YZfz),(Xe,Cd,YZga), (Xe,Cd,YZgb),(Xe,Cd,YZgc),(Xe,Cd,YZgd),(Xe,Cd,YZge),(Xe,Cd,YZgf),(Xe,Cd,YZgg), (Xe,Cd,YZgh),(Xe,Cd,YZgi),(Xe,Cd,YZgj),(Xe,Cd,YZgk),(Xe,Cd,YZgl),(Xe,Cd, YZgm),(Xe,Cd,YZgn),(Xe,Cd,YZgo),(Xe,Cd,YZgp),(Xe,Cd,YZgq),(Xe,Cd,YZgr),(Xe, Cd,YZgs),(Xe,Cd,YZgt),(Xe,Ce,YZa),(Xe,Ce,YZb),(Xe,Ce,YZc),(Xe,Ce,YZd),(Xe,Ce, YZe),(Xe,Ce,YZf),(Xe,Ce,YZg),(Xe,Ce,YZh),(Xe,Ce,YZi),(Xe,Ce,YZj),(Xe,Ce,YZk), (Xe,Ce,YZl),(Xe,Ce,YZm),(Xe,Ce,YZn),(Xe,Ce,YZo),(Xe,Ce,YZp),(Xe,Ce,YZq),(Xe, Cc,YZr),(Xe,Ce,YZs),(Xe,Ce,YZt),(Xe,Ce,YZu),(Xe,Ce,YZv),(Xe,Ce,YZw),(Xe,Ce, YZx),(Xe,Ce,YZy),(Xe,Ce,YZz),(Xe,Ce,YZaa),(Xe,Ce,YZab),(Xe,Ce,YZac),(Xe,Ce, YZad),(Xe,Ce,YZae),(Xe,Ce,YZaf),(Xe,Ce,YZag),(Xe,Ce,YZah),(Xe,Ce,YZai),(Xe,Ce, YZaj),(Xe,Ce,YZak),(Xe,Ce,YZal),(Xe,Ce,YZam),(Xe,Ce,YZan),(Xe,Ce,YZao),(Xc, Ce,YZap),(Xe,Ce,YZaq),(Xe,Ce,YZar),(Xe,Ce,YZas),(Xe,Ce,YZat),(Xe,Ce,YZau),(Xe, Ce,YZav),(Xe,Ce,YZaw),(Xe,Ce,YZax),(Xe,Ce,YZay),(Xe,Ce,YZaz),(Xe,Ce,YZba), (Xe,Ce,YZbb),(Xe,Ce,YZbc),(Xe,Ce,YZbd),(Xe,Ce,YZbe),(Xe,Ce,YZbf),(Xe,Ce,YZbg), (Xe,Ce,YZbh),(Xe,Ce,YZbi),(Xe,Ce,YZbj),(Xe,Ce,YZbk),(Xe,Ce,YZbl),(Xe,Ce,YZbm), (Xe,Ce,YZbn),(Xe,Ce,YZbo),(Xe,Ce,YZbp),(Xe,Ce,YZbq),(Xe,Ce,YZbr),(Xe,Ce, YZbs),(Xe,Ce,YZbt),(Xe,Ce,YZbu),(Xe,Ce,YZbv),(Xe,Ce,YZbw),(Xe,Ce,YZbx),(Xe, Ce,YZby),(Xe,Ce,YZbz),(Xe,Ce,YZca),(Xe,Ce,YZcb),(Xe,Ce,YZcc),(Xe,Ce,YZcd),(Xe, Ce,YZce),(Xe,Ce,YZcf),(Xe,Ce,YZcg),(Xe,Ce,YZch),(Xe,Ce,YZci),(Xe,Ce,YZcj), (Xe,Ce,YZck),(Xe,Ce,YZcl),(Xe,Ce,YZcm),(Xe,Ce,YZen),(Xe,Ce,YZco),(Xe,Ce,YZcp), (Xe,Ce,YZcq),(Xe,Ce,YZcr),(Xe,Ce,YZcs),(Xe,Ce,YZct),(Xe,Ce,YZcu),(Xe,Ce,YZcv), (Xe,Ce,YZcw),(Xe,Ce,YZcx),(Xe,Ce,YZcy),(Xe,Ce,YZcz),(Xe,Ce,YZda),(Xe,Ce, YZdb),(Xe,Ce,YZdc),(Xe,Ce,YZdd),(Xe,Ce,YZde),(Xe,Ce,YZdf),(Xe,Ce,YZdg),(Xe, Ce,YZdh),(Xe,Ce,YZdi),(Xe,Ce,YZdj),(Xe,Ce,YZdk),(Xe,Ce,YZdl),(Xe,Ce,YZdm),(Xe, Ce,YZdn),(Xe,Ce,YZdo),(Xe,Ce,YZdp),(Xe,Ce,YZdq),(Xe,Ce,YZdr),(Xe,Ce,YZds), (Xe,Ce,Yzdt),(Xe,Ce,YZdu),(Xe,Ce,YZdv),(Xe,Ce,YZdw),(Xe,Ce,YZdx),(Xe,Ce,YZdy), (Xe,Ce,YZdz),(Xe,Ce,YZea),(Xe,Ce,YZeb),(Xe,Ce,YZec),(Xe,Ce,YZed),(Xe,Ce, YZee),(Xe,Ce,YZef),(Xe,Ce,YZeg),(Xe,Ce,YZeh),(Xe,Ce,YZei),(Xe,Cc,YZej),(Xe,Ce, YZek),(Xe,Ce,YZel),(Xe,Ce,YZem),(Xe,Ce,YZen),(Xe,Ce,YZeo),(Xe,Ce,YZep),(Xe, Ce,YZeq),(Xe,Ce,YZer),(Xe,Ce,YZes),(Xe,Ce,YZet),(Xe,Ce,YZeu),(Xe,Ce,YZev),(Xe, Ce,YZew),(Xe,Ce,YZex),(Xe,Ce,YZey),(Xe,Ce,YZez),(Xe,Ce,YZfa),(Xe,Ce,YZfb),(Xe, Ce,YZfc),(Xe,Ce,YZfd),(Xe,Ce,YZfe),(Xe,Ce,YZff),(Xe,Ce,YZfg),(Xe,Ce,YZfh),(Xe, Ce,YZfi),(Xe,Ce,YZfj),(Xe,Ce,YZfk),(Xe,Ce,YZfl),(Xe,Ce,YZfm),(Xe,Ce,YZfn),(Xe, Ce,YZfo),(Xe,Ce,YZfp),(Xe,Ce,YZfq),(Xe,Ce,YZfr),(Xe,Ce,YZfs),(Xe,Ce,YZft),(Xe, Ce,YZfau),(Xe,Ce,YZfv),(Xe,Ce,YZfw),(Xe,Ce,YZfx),(Xe,Ce,YZfy),(Xe,Ce,YZfz), (Xe,Ce,YZga),(Xe,Ce,YZgb),(Xe,Ce,YZgc),(Xe,Ce,YZgd),(Xe,Ce,YZge),(Xe,Ce,YZgf), (Xe,Ce,YZgg),(Xe,Ce,YZgh),(Xe,Ce,YZgi),(Xe,Ce,YZgj),(Xe,Ce,YZgk),(Xe,Ce,YZgl), (Xe,Ce,YZgm),(Xe,Ce,YZgn),(Xe,Ce,YZgo),(Xe,Ce,YZgp),(Xe,Ce,YZgq),(Xe,Ce, YZgr),(Xe,Ce,YZgs),(Xe,Ce,YZgt),(Xe,Cf,YZa),(Xe,Cf,YZb),(Xe,Cf,YZc),(Xe,Cf, YZd),(Xe,Cf,YZe),(Xe,Cf,YZf),(Xe,Cf,YZg),(Xe,Cf,YZh),(Xe,Cf,YZi),(Xe,Cf,YZj),(Xe, Cf,YZk),(Xe,Cf,YZl),(Xe,Cf,YZm),(Xe,Cf,YZn),(Xe,Cf,YZo),(Xe,Cf,YZp),(Xe,Cf, YZq),(Xe,Cf,YZr),(XSe,Cf,YZs),(Xe,Cf,YZt),(Xe,Cf,YZu),(Xe,Cf,YZv),(Xe,Cf,YZw), (Xe,Cf,YZx),(Xe,Cf,YZy),(Xe,Cf,YZz),(Xe,Cf,YZaa),(Xe,Cf,YZab),(Xe,Cf,YZac),(Xe, Cf,YZad),(Xe,Cf,YZae),(Xe,Cf,YZaf),(Xe,Cf,YZag),(,Xe,Cf,YZah),(Xe,Cf,YZai),(Xe, Cf,YZaj),(Xe,Cf,YZak),(Xe,Cf,YZal),(Xe,Cf,YZm),(Xe,Cf,YZan),(Xe,Cf,YZao),(Xe, Cf,YZap),(Xe,Cf,YZaq),(Xe,Cf,YZar),(Xe,Cf,YZas),(Xe,Cf,YZat),(Xe,Cf,YZau),(Xe, Cf,YZav),(Xe,Cf,YZaw),(Xe,Cf,YZax),(Xe,Cf,YZay),(Xe,Cf,YZaz),(Xe,Cf,YZba),(Xe, Cf,YZbb),(Xe,Cf,YZbc),(Xe,Cf,YZbd),(Xe,Cf,YZbe),(Xe,Cf,YZbf),(Xe,Cf,YZbg), (Xe,Cf,YZbh),(Xe,Cf,YZbi),(Xe,Cf,YZbj),(Xe,Cf,YZbk),(Xe,Cf,YZbl),(Xe,Cf,YZbm), (Xe,Cf,YZbn),(Xe,Cf,YZbo),(Xe,Cf,YZbp),(Xe,Cf,YZbq),(Xe,Cf,YZbr),(Xe,Cf,YZbs), (Xe,Cf,YZbt),(Xe,Cf,YZbu),(Xe,Cf,YZbv),(Xe,Cf,YZbw),(Xe,Cf,YZbx),(Xe,Cf,YZby), (Xe,Cf,YZbz),(Xe,Cf,YZca),(Xe,Cf,YZcb),(Xe,Cf,YZcc),(Xe,Cf,YZcd),(Xe,Cf,YZce), (Xe,Cf,YZcf),(Xe,Cf,YZcg),(Xe,Cf,YZch),(Xe,Cf,YZci),(Xe,Cf,YZcj),(Xe,Cf,YZck), (Xe,Cf,YZcl),(Xe,Cf,YZcm),(Xe,Cf,YZcn),(Xe,Cf,YZco),(Xe,Cf,YZcp),(Xe,Cf,YZcq), (Xe,Cf,YZcr),(Xe,Cf,YZcs),(Xe,Cf,YZct),(Xe,Cf,YZcu),(Xe,Cf,YZcv),(Xe,Cf,YZcw), (Xe,Cf,YZcx),(Xe,Cf,YZcy),(Xe,Cf,YZcz),(Xe,Cf,YZda),(Xe,Cf,YZdb),(Xe,Cf,YZdc), (Xe,Cf,YZdd),Xxe,Cf,YZde),(Xe,Cf,YZdf),(Xe,Cf,YZdg),(Xe,Cf,YZdh),(Xe,Cf, YZdi),(Xe,Cf,YZdj),(Xe,Cf,YZdk),(Xe,Cf,YZdl),(Xe,Cf,YZdm),(Xe,Cf,YZdn),(Xe,Cf, YZdo),(Xe,Cf,YZdp),(Xe,Cf,YZdq),(Xe,Cf,YZdr),(Xe,Cf,YZds),(Xe,Cf,Yzdt),(Xe,Cf, YZdu),(Xe,Cf,YZdv),(Xe,Cf,YZdw),(Xe,Cf,YZdx),(Xe,Cf,YZdy),(Xe,Cf,YZdz),(Xe, Cf,YZea),(Xe,Cf,YZeb),(Xe,Cf,YZec),(Xe,Cf,YZed),(Xe,Cf,YZee),(Xe,Cf,YZef),(Xe, Cf,YZeg),(Xe,Cf,YZeh),(Xe,Cf,YZei),(Xe,Cf,YZej),(Xe,Cf,YZek),(Xe,Cf,YZel),(Xe, Cf,YZem),(Xe,Cf,YZen),(Xe,Cf,YZeo),(Xe,Cf,YZep),(Xe,Cf,YZeq),(Xe,Cf,YZer),(Xe, Cf,YZes),(Xe,Cf,YZet),(Xe,Cf,YZeu),(Xe,Cf,YZev),(Xe,Cf,YZew),(Xe,Cf,YZex),(Xe, Cf,YZey),(Xe,Cf,YZez),(Xe,Cf,YZfa),(Xe,Cf,YZfb),(Xe,Cf,YZfc),(Xe,Cf,YZfd),(Xe, Cf,YZfe),(Xe,Cf,YZff),(Xe,Cf,YZfg),(Xe,Cf,YZfh),(Xe,Cf,YZfi),(Xe,Cf,YZfj),(Xe, Cf,YZfk),(Xe,Cf,YZfl),(Xe,Cf,YZfm),(Xe,Cf,YZfn),(Xe,Cf,YZfo),(Xe,Cf,YZfp),(Xe, Cf,YZfq),(Xe,Cf,YZfr),(Xe,Cf,YZfs),(Xe,Cf,YZft),(Xe,Cf,YZfu),(Xe,Cf,YZfv),(Xe,Cf, YZfw),(Xe,Cf,YZfx),(Xe,Cf,YZfy),(Xe,Cf,YZfz),(Xe.Cf,YZga),(Xe,Cf,YZgb),(Xe,Cf, YZgc),(Xe,Cf,YZgd),(Xe,Cf,YZge),(Xe,Cf,YZgf),(Xe,Cf,YZgg),(Xe,Cf,YZgh),(Xe,Cf, YZgi),(Xe,Cf,YZgj),(Xe,Cf,YZgk),(Xe,Cf,YZgl),(Xe,Cf,YZgm),(Xe,Cf,YZgn),(Xe, Cf,YZgo),(Xe,Cf,YZgp),(Xe,Cf,YZgq),(Xe,Cf,YZgr),(Xe,Cf,YZgs),(Xe,Cf,YZgt), (Xcf,Ca,YZa),(Xf,Ca,YZb),(Xf,Ca,YZc),(Xf,Ca,YZd),(Xf,Ca,YZe),(Xf,Ca,YZf),(Xf,Ca, YZg),(Xf,Ca,YZh),(Xf,Ca,YZi),(Xf,Ca,YZj),(Xf,Ca,YZk),(Xf,Ca,YZl),(Xf,Ca,YZm), (Xf,Ca,YZn),(Xf,Ca,YZo),(Xf,Ca,YZp),(Xf,Ca,YZq),(Xf,Ca,YZr),(Xf,Ca,YZs),(Xf,Ca, YZt),(Xf,Ca,YZu),(Xf,Ca,YZv),(Xf,Ca,YZw),(Xf,Ca,YZx),(Xf,Ca,YZy),(Xf,Ca,YZz), (Xf,Ca,YZaa),(Xf,Ca,YZab),(Xf,Ca,YZac),(Xf,Ca,YZad),(Xf,Ca,YZae),(Xf,Ca,YZaf), (Xf,Ca,YZag),(Xf,Ca,YZah),(Xf,Ca,YZai),(Xf,Ca,YZaj),(Xf,Ca,YZak),(Xf,Ca,YZal), (Xf,Ca,YZam),(Xf,Ca,YZan),(Xf,Ca,YZao),(Xf,Ca,YZap),(Xf,Ca,YZaq),(Xf,Ca,YZar, (Xf,Ca,YZas),(Xf,Ca,YZat),(Xf,Ca,YZau),(Xf,Ca,YZav),(Xf,Ca,YZaw),(Xf,Ca,YZax), (Xf,Ca,YZay),(Xf,Ca,YZaz),(Xf,Ca,YZba),(Xf,Ca,YZbb),(Xf,Ca,YZbc),(Xf,Ca,YZbd), (Xf,Ca,YZbe),(Xf,Ca,YZbf),(Xf,Ca,YZbg),(Xf,Ca,YZbh),(Xf,Ca,YZbi),(Xf,Ca,YZbj), (Xf,Ca,YZbk),(Xf,Ca,YZbl),(Xf,Ca,YZbm),(Xf,Ca,YZbn),(Xf,Ca,YZbo),(Xf,Ca, YZbp),(Xf,Ca,YZbq),(Xf,Ca,YZbr),(Xf,Ca,YZbs),(Xf,Ca,YZbt),(Xf,Ca,YZbu),(Xf,Ca, YZbv),(Xf,Ca,YZbw),(Xf,Ca,YZbx),(Xf,Ca,YZby),(Xf,Ca,YZbz),(Xf,Ca,YZca),(Xf,Ca, YZcb),(Xf,Ca,YZcc),(Xf,Ca,YZcd),(Xf,Ca,YZce),(Xf,Ca,YZcf),(Xf,Ca,YZcg),(Xf,Ca, YZch),(Xf,Ca,YZci),(Xf,Ca,YZcj),(Xf,Ca,YZck),(Xf,Ca,YZcl),(Xf,Ca,YZcm),(Xf,Ca, YZcn),(Xf,Ca,YZco),(Xf,Ca,YZcp),(Xf,Ca,YZcq),(Xf,Ca,YZcr),(Xf,Ca,YZcs),(Xf,Ca, YZct),(Xf,Ca,YZcu),(Xf,Ca,YZcv),(Xf,Ca,YZcw),(Xf,Ca,YZcx),(Xf,Ca,YZcy),(Xf, Ca,YZcz),(Xf,Ca,YZda),(Xf,Ca,YZdb),(Xf,Ca,YZdc),(Xf,Ca,YZdd),(Xf,Ca,YZdc),(Xf, Ca,YZdf),(Xf,Ca,YZdg),(Xf,Ca,YZdh),(Xf,Ca,YZdi),(Xf,Ca,YZdj),(Xf,Ca,YZdk),(Xf, Ca,YZdl),(Xf,Ca,YZdm),(Xf,Ca,YZdn),(Xf,Ca,YZdo),(Xf,Ca,YZdp),(Xf,Ca,YZdq),(Xf, Ca,YZdr),(Xf,Ca,YZds),(Xf,Ca,Yzdt),(Xf,Ca,YZdu),(Xf,Ca,YZdv),(Xf,Ca,YZdw), (Xf,Ca,YZdx),(Xf,Ca,YZdy),(Xf,Ca,YZdz),(Xf,Ca,YZea),(Xf,Ca,YZeb),(Xf,Ca,YZec), (Xf,Ca,YZed),(Xf,Ca,YZee),(Xf,Ca,YZef),(Xf,Ca,YZeg),(Xf,Ca,YZeh),(Xf,Ca,YZei), (Xf,Ca,YZej),(Xf,Ca,YZek),(Xf,Ca,YZel),(Xf,Ca,YZem),(Xf,Ca,YZen),(Xf,Ca,YZeo), (Xf,Ca,YZep),(Xf,Ca,YZeq),(Xf,Ca,YZer),(Xf,Ca,YZes),(Xf,Ca,YZet),(Xf,Ca,YZeu),(Xf, Ca,YZev),(Xf,Ca,YZew),(Xf,Ca,YZex),(Xf,Ca,YZey),(Xf,Ca,YZez),(Xf,Ca,YZfa), (Xf,Ca,YZfb),(Xf,Ca,YZfc),(Xf,Ca,YZfd),(Xf,Ca,YZfe),(Xf,Ca,YZff),(Xf,Ca,YZfg),(Xf, Ca,YZfh),(Xf,Ca,YZfi),(Xf,Ca,YZfj),(Xf,Ca,YZfk),(Xf,Ca,YZfl),(Xf,Ca,YZfm),(Xf, Ca,YZfn),(Xf,Ca,YZfo),(Xf,Ca,YZfp),(Nf,Ca,YZfq),(Xf,Ca,YZfr),(Xf,Ca,YZfs),(Xf, Ca,YZft),(Xf,Ca,YZfu),(Xf,Ca,YZfv),(Xf,Ca,YZfw),(Xf,Ca,YZfx),(Xf,Ca,YZfy),(Xf, Ca,YZfz),(Xf,Ca,YZga),(Xf,Ca,YZgb),(Xf,Ca,YZgc),(Xf,Ca,YZgd),(Xf,Ca,YZge),(Xf, Ca,YZgf),(Xf,Ca,YZgg),(Xf,Ca,YZgh),(Xf,Ca,YZgi),(Xf,Ca,YZgj),(Xf,Ca,YZgk),(Xf, Ca,YZgl),(Xf,Ca,YZgm),(Xf,Ca,YZgn),(Xf,Ca,YZgo),(Xf,Ca,YZgp),(Xf,Ca,YZgq),(Xf, Ca,YZgr),(Xf,Ca,YZgs),(Xf,Ca,YZgt),(Xf,Cb,YZa),(Xf,Cb,YZb),(Xf,Cb,YZc),(Xf, Cb,YZd),(Xf,Cb,YZe),(Xf,Cb,YZf),(Xf,Cb,YZg),(Xf,Cb,YZh),(Xf,Cb,YZi),(Xf,Cb,YZj), (Xf,Cb,YZk),(Xf,Cb,YZl),(Xf,Cb,YZm),(Xf,Cb,YZn),(Xf,Cb,YZo),(Xf,Cb,YZp),(Xf, Cb,YZq),(Xf,Cb,YZr),(Xf,Cb,YZs),(Xf,Cb,YZt),(Xf,Cb,YZu),(Xf,Cb,YZv),(Xf,Cb, YZw),(Xf,Cb,YZx),(Xf,Cb,YZy),(Xf,Cb,YZz),(Xf,Cb,YZaa),(Xf,Cb,YZab),(Xf,Cb,YZac), (Xf,Cb,YZad),(Xf,Cb,YZae),(Xf,Cb,YZaf),(Xf,Cb,YZag),(Xf,Cb,YZah),(Xf,Cb, YZai),(Xf,Cb,YZaj),(Xf,Cb,YZak),(Xf,Cb,YZal),(Xf,Cb,YZam),(Xf,Cb,YZan),(Xf,Cb, YZao),(Xf,Cb,YZap),(Xf,Cb,YZaq),(Xf,Cb,YZar),(Xf,Cb,YZas),(Xf,Cb,YZat),(Xf,Cb, YZau),(Xf,Cb,YZav),(Xf,Cb,YZaw),(Xf,Cb,YZax),(Xf,Cb,YZay),(Xf,Cb,YZaz),(Xf, Cb,YZba),(Xf,Cb,YZbb),(Xf,Cb,YZbc),(Xf,Cb,YZbd),(Xf,Cb,YZbe),(Xf,Cb,YZbf),(Xf, Cb,YZbg),(Xf,Cb,YZbh),(Xf,Cb,YZbi),(Xf,Cb,YZbj),(Xf,Cb,YZbk),(Xf,Cb,YZbl),(Xf, Cb,YZbm),(Xf,Cb,YZbn),(Xf,Cb,YZbo),(Xf,Cb,YZbp),(Xf,Cb,YZbq),(Xf,Cb,YZbr), (Xf,Cb,YZbs),(Xf,Cb,YZbt),(Xf,Cb,YZbu),(Xf,Cb,YZbv),(Xf,Cb,YZbw),(Xf,Cb,YZbx), (Xf,Cb,YZby),(Xf,Cb,YZbz),(Xf,Cb,YZca),(Xf,Cb,YZcb),(Xf,Cb,YZcc),(Xf,Cb, YZcd),(Xf,Cb,YZce),(Xf,Cb,YZef),(Xf,Cb,YZcg),(Xf,Cb,YZch),(Xf,Cb,YZci),(Xf,Cb, YZcj),(Xf,Cb,YZck),(Xf,Cb,YZcl),(Xf,Cb,YZcm),(Xf,Cb,YZcn),(Xf,Cb,YZco),(Xf,Cb, YZcp),(Xf,Cb,YZcq),(Xf,Cb,YZer),(Xf,Cb,YZcs),(Xf,Cb,YZct),(Xf,Cb,YZcu),(Xf,Cb, YZcv),(Xf,Cb,YZcw),(Xf,Cb,YZcx),(Xf,Cb,YZey),(Xf,Cb,YZcz),(Xf,Cb,YZda),(Xf, Cb,YZdb),(Xf,Cb,YZdc),(Xf,Cb,YZdd),(Xf,Cb,YZde),(Xf,Cb,YZdf),(Xf,Cb,YZdg),(Xf, Cb,YZdh),(Xf,Cb,YZdi),(Xf,Cb,YZdj),(Xf,Cb,YZdk),(Xf,Cb,YZdl),(Xf,Cb,YZdm),(Xf, Cb,YZdn),(Xf,Cb,YZdo),(Xf,Cb,YZdp),(Xf,Cb,YZdq),(Xf,Cb,YZdr),(Xf,Cb,YZds), (Xf,Cb,Yzdt),(Xf,Cb,YZdu),(Xf,Cb,YZdv),(Xf,Cb,YZdw),(Xf,Cb,Ydx),(Xf,Cb,YZdy), (Xf,Cb,YZdz),(Xf,Cb,YZea),(Xf,Cb,YZeb),(Xf,Cb,YZec),(Xf,Cb,YZed),(Xf,Cb,YZee), (Xf,Cb,YZef),(Xf,Cb,YZeg),(Xf,Cb,YZeh),(Xf,Cb,YZei),(Xf,Cb,YZej),(Xf,Cb,YZek), (Xf,Cb,YZel),(Xf,Cb,YZem),(Xf,Cb,YZen),(Xf,Cb,YZeo),(Xf,Cb,YZep),(Xf,Cb,YZeq), (Xf,Cb,YZer),(Xf,Cb,YZes),(Xf,Cb,YZet),(Xf,Cb,YZeu),(Xf,Cb,YZev),(Xf,Cb,YZew), (Xf,Cb,YZex),(Xf,Cb,YZey),(Xf,Cb,YZez),(Xf,Cb,YZfa),(Xf,Cb,YZfb),(Xf,Cb, YZfc),(Xf,Cb,YZfd),(Xf,Cb,YZfe),(Xf,Cb,YZff),(Xf,Cb,YZfg),(Xf,Cb,YZfh),(Xf,Cb, YZfi),(Xf,Cb,YZfj),(Xf,Cb,YZfk),(Xf,Cb,YZfl),(Xf,Cb,YZfm),(Xf,Cb,YZfn),(Xf,Cb, YZfo),(Xf,Cb,YZfp),(Xf,Cb,YZfq),(Xf,Cb,YZfr),(Xf,Cb,YZfs),(Xf,Cb,YZft),(Xf,Cb, YZfu),(Xf,Cb,YZfv),(Xf,Cb,YZfw),(Xf,Cb,YZfx),(Xf,Cb,YZfy),(Xf,Cb,YZfz),(Xf,Cb, YZga),(Xf,Cb,YZgb),(Xf,Cb,YZgc),(Xf,Cb,YZgd),(Xf,Cb,YZge),(Xf,Cb,YZgf,(Xf,Cb, YZgg),(Xf,Cb,YZgh),(Xf,Cb,YZgi),(Xf,Cb,YZgj),(Xf,Cb,YZgk),(Xf,Cb,YZgl),(Xf, Cb,YZgm),(Xf,Cb,YZgn),(Xf,Cb,YZgo),(Xf,Cb,YZgp),(Xf,Cb,YZgq),(Xf,Cb,YZgr),(Xf, Cb,YZgs),(Xf,Cb,YZgt),(Xf,Cc,YZa),(Xf,Cc,YZb),(Xf,Cc,YZc),(Xf,Cc,YZd),(Xf, Cc,YZe),(Xf,Cc,YZf),(Xf,Cc,YZg),(Xf,Cc,YZh),(Xf,Cc,YZi),(Xf,Cc,YZj),(Xf,Cc,YZk), (Xf,Cc,YZl),(Xf,Cc,YZm),(Xf,Cc,YZn),(Xf,Cc,YZo),(Xf,Cc,YZp),(Xf,Cc,YZq),(Xf, Cc,YZr),(Xf,Cc,YZs),(Xf,Cc,YZt),(Xf,Cc,YZu),(Xf,Cc,YZv),(Xf,Cc,YZw),(Xf,Cc,YZx), (Xf,Cc,YZy),(Xf,Cc,YZz),(Xf,Cc,YZaa),(Xf,Cc,YZab),(Xf,Cc,YZac),(Xf,Cc,YZad), (Xf,Cc,YZae),(Xf,Cc,YZaf),(Xf,Cc,YZag),(Xf,Cc,YZah),(Xf,Cc,YZai),(Xf,Cc,YZaj), (Xf,Cc,YZak),(Xf,Cc,YZal),(Xf,Cc,Y/am),(Xf,Cc,YZan),(Xf,Cc,YZao),(Xf,Cc,YZap), (Xf,Cc,YZaq),(Xf,Cc,YZar),(Xf,Cc,YZas),(Xf,Cc,YZat),(Xf,Cc,YZau),(Xf,Ce,YZav), (Xf,Cc,YZaw),(Xf,Cc,YZax),(Xf,Cc,YZay),(Xf,Cc,YZaz),(Xf,Cc,YZba),(Xf,Cc,YZbb), (Xf,Cc,YZbc),(Xf,Cc,YZbd),(Xf,Cc,YZbe),(Xf,Cc,YZbf),(Xf,Cc,YZbg),(Xf,Cc,YZbh), (Xf,Cc,YZbi),(Xf,Cc,YZbj),(Xf,Cc,YZbk),(Xf,Cc,YZbl),(Xf,Cc,YZbm),(Xf,Cc, YZbn),(Xf,Cc,YZbo),(Xf,Cc,YZbp),(Xf,Cc,YZbq),(Xf,Cc,YZbr),(Xf,Cc,YZbs),(Xf,Cc, YZbt),(Xf,Cc,YZbu),(Xf,Cc,YZbv),(Xf,Cc,YZbw),(Xf,Cc,YZbx),(Xf,Cc,YZby),(Xf, Cc,YZbz),(Xf,Cc,YZca),(Xf,Cc,YZcb),(Xf,Cc,YZcc),(Xf,Cc,YZcd),(Xf,Cc,YZce),(Xf, Cc,YZcf),(Xf,Cc,YZcg),(Xf,Cc,YZch),(Xf,Cc,YZci),(Xf,Cc,YZcj),(Xf,Cc,YZck),(Xf, Cc,YZcl),(Xf,Cc,YZcm),(Xf,Cc,YZcn),(Xf,Cc,YZco),(Xf,Cc,YZcp),(Xf,Cc,YZcq),(Xf, Cc,YZcr),(Xf,Cc,YZcs),(Xf,Cc,YZct),(Xf,Cc,YZcu),(Xf,Cc,YZcv),(Xf,Cc,YZcw),(Xf, Cc,YZcx),(Xf,Cc,YZcy),(Xf,Cc,YZcz),(Xf,Cc,YZda),(Xf,Cc,YZdb),(Xf,Cc,YZdc),(Xf, Cc,YZdd),(Xf,Cc,YZde),(Xf,Cc,YZdf),(Xf,Cc,YZdg),(Xf,Cc,YZdh),(Xf,Cc,YZdi),(Xf, Cc,YZdj),(Xf,Cc,YZdk),(Xf,Cc,YZdl),(Xf,Cc,YZdm),(Xf,Cc,YZdnl),(Xf,Cc,YZdo), (Xf,Cc,YZdp),(Xf,Cc,YZdq),(Xf,Cc,YZdr),(Xf,Cc,YZds),(Xf,Cc,Yzdt),(Xf,Cc,YZdu), (Xf,Cc,YZdv),(Xf,Cc,YZdw),(Xf,Cc,YZdx),(Xf,Cc,YZdy),(Xf,Cc,YZdz),(Xf,Cc,YZea), (Xf,Cc,YZeb),(Xf,Cc,YZec),(Xf,Cc,YZed),(Xf,Cc,YZee),(Xf,Cc,YZef),(Xf,Cc,YZeg), (Xf,Cc,YZeh),(Xf,Cc,YZei),(Xf,Cc,YZej),(Xf,Cc,YZek),(Xf,Cc,YZel),(Xf,Cc,YZem), (Xf,Cc,YZen),(Xf,Cc,YZeo),(Xf,Cc,YZep),(Xf,Cc,YZeq),(Xf,Cc,YZer),(Xf,Cc,YZes), (Xf,Cc,yZet),(Xf,Cc,yZeu),(Xf,Cc,YZev),(Xf,CG,YZew),(Xf,Cc,YZex),(Xf,Cc,YZey), (Xf,Cc,YZez),(Xf,Cc,YZfa),(Xf,Cc,YZfb),(Xf,Cc,YZfc),(Xf,Cc,YZfd),(Xf,Cc,YZfe), (Xf,Cc,YZff),(Xf,Cc,YZfg),(Xf,Cc,YZfh),(Xf,Cc,YZfi),(Xf,Cc,YZfj),(Xf,Cc,YZfk),(Xf, Cc,YZfl),(Xf,Cc,YZfm),(Xf,Cc,YZfn),(Xf,Cc,YZfo),(Xf,Cc,YZfp),(Xf,Cc,YZfq),(Xf, Cc,YZfr),(Xf,Cc,YZfs),(Xf,Cc,YZft),(Xf,Cc,YZfu),(Xf,Cc,YZfv),(Xf,Cc,YZfw),(Xf, Cc,YZfx),(Xf,Cc,YZfy),(Xf,Cc,YZfz),(Xf,Cc,YZga),(Xf,Cc,YZgb),(Xf,Cc,YZge),(Xf, Cc,YZgd),(Xf,Cc,YZge),(Xf,Cc,YZgf),(Xf,Cc,YZgg),(Xf,Cc,YZgh),(Xf,Cc,YZgi),(Xf, Cc,YZgj),(Xf,Cc,YZgk),(Xf,Cc,YZgl),(Xf,Cc,YZgm),(Xf,Cc,YZgn),(Xf,Cc,YZgo), (Xf,Cc,YZgp),(Xf,Cc,YZgq),(Xf,Cc,YZgr),(Xf,Cc,YZgs),(Xf,Cc,YZgt),(Xf,Cd,YZa),(Xf, Cd,YZb),(Xf,Cd,YZc),(Xf,Cd,YZd),(Xf,Cd,YZe),(Xf,Cd,YZf),(Xf,Cd,YZg),(Xf,Cd, YZh),(Xf,Cd,YZi),(Xf,Cd,YZj),(Xf,Cd,YZk),(Xf,Cd,YZl),(Xf,Cd,YZm),(Xf,Cd,YZn), (Xf,Cd,YZo),(Xf,Cd,YZp),(Xf,Cd,YZq),(Xf,Cd,YZr),(Xf,Cd,YZs),(Xf,Cd,YZt),(Xf, Cd,YZu),(Xf,Cd,YZv),(Xf,Cd,YZw),(Xf,Cd,YZx),(Xf,Cd,YZy),(Xf,Cd,YZz),(Xf,Cd, YZaa),(Xf,Cd,YZab),(Xf,Cd,YZac),(Xf,Cd,YZad),(Xf,Cd,YZae),(Xf,Cd,YZaf),(Xf,Cd, YZag),(Xf,Cd,YZah),(Xf,Cd,YZai),(Xf,Cd,YZaj),(Xf,Cd,YZak),(Xf,Cd,YZal),(Xf,Cd, YZam),(XfE,Cd,YZan),(Xf,Cd,YZao),(Xf,Cd,YZap),(Xf,Cd,YZaq),(Xf,Cd,YZar),(Xf, Cd,YZas),(Xf,Cd,YZat),(Xf,Cd,YZau),(Xf,Cd,YZav),(Xf,Cd,YZaw),(Xf,Cd,YZax),(Xf, Cd,YZay),(Xf,Cd,YZaz),(Xf,Cd,YZba),(Xf,Cd,YZbb),(Xf,Cd,YZbc),(Xf,Cd,YZbd),(Xf, Cd,YZbe),(Xf,Cd,YZbf),(Xf,Cd,YZbg),(Xf,Cd,YZbh),(Xf,Cd,YZbi),(Xf,Cd,YZbj), (Xf,Cd,YZbk),(Xf,Cd,YZbl),(Xf,Cd,YZbm),(Xf,Cd,YZbn),(Xf,Cd,YZbo),(Xf,Cd,YZbp), (Xf,Cd,YZbq),(Xf,Cd,YZbr),(Xf,Cd,YZbs),(Xf,Cd,YZbt),(Xf,Cd,YZbu),(Xf,Cd,YZbv), (Xf,Cd,YZbw),(Xf,Cd,YZbx),(Xf,Cd,YZby),(Xf,Cd,YZbz),(Xf,Cd,YZca),(Xf,Cd, YZcb),(Xf,Cd,YZcc),(Xf,Cd,YZcd),(Xf,Cd,YZce),(Xf,Cd,YZcf),(Xf,Cd,YZcg),(Xf,Cd, YZch),(Xf,Cd,YZci),(Xf,Cd,YZcj),(Xf,Cd,YZck),(Xf,Cd,YZcl),(Xf,Cd,YZcm),(Xf,Cd, YZcn),(Xf,Cd,YZco),(Xf,Cd,YZcp),(Xf,Cd,YZcq),(Xf,Cd,YZcr),(Xf,Cd,YZcs),(Xf, Cd,YZct),(Xf,Cd,YZcu),(Xf,Cd,YZcv),(Xf,Cd,YZcw),(Xf,Cd,YZcx),(Xf,Cd,YZcy),(Xf, Cd,YZcz),(Xf,Cd,YZda),(Xf,Cd,YZdb),(Xf,Cd,YZdc),(Xf,Cd,YZdd),(Xf,Cd,YZde),(Xf, Cd,YZdf),(Xf,Cd,YZdg),(Xf,Cd,YZdh),(Xf,Cd,YZdi),(Xf,Cd,YZdj),(Xf,Cd,YZdk), (Xf,Cd,YZdl),(Xf,Cd,YZdm),(Xf,Cd,YZdn),(Xf,Cd,YZdo),(Xf,Cd,YZdp),(Xf,Cd,YZdq), (Xf,Cd,YZdr),(Xf,Cd,YZds),(Xf,Cd,Yzdt),(Xf,Cd,YZdu),(Xf,Cd,YZdv),(Xf,Cd,YZdw), (Xf,Cd,YZdx),(Xf,Cd,YZdy),(Xf,Cd,YZdz),(Xf,Cd,YZea),(Xf,Cd,YZeb),(Xf,Cd, YZec),(Xf,Cd,YZed),(Xf,Cd,YZee),(Xf,Cd,YZef),(Xf,Cd,YZeg),(Xf,Cd,YZeh),(Xf,Cd, YZei),(Xf,Cd,YZej),(Xf,Cd,YZek),(Xf,Cd,YZel),(Xf,Cd,YZem),(Xf,Cd,YZen),(Xf,Cd, YZeo),(Xf,Cd,YZep),(Xf,Cd,YZeq),(Xf,Cd,YZer),(Xf,Cd,YZes),(Xf,Cd,YZet),(Xf,Cd, YZeu),(Xf,Cd,YZev),(Xf,Cd,YZew),(Xf,Cd,YZex),(Xf,Cd,YZey),(Xf,Cd,YZez),(Xf, Cd,YZfa),(Xf,Cd,YZfb),(Xf,Cd,YZfc),(Xf,Cd,YZfd),(Xf,Cd,YZfe),(Xf,Cd,YZff),(Xf, Cd,YZfg),(Xf,Cd,YZfh),(Xf,Cd,YZfi),(Xf,Cd,YZfj),(Xf,Cd,YZfk),(Xf,Cd,YZfl),(Xf,Cd, YZfm),(Xf,Cd,YZfn),(Xf,Cd,YZfo),(Xf,Cd,YZfp),(Xf,Cd,YZfq),(Xf,Cd,YZfr),(Xf,Cd, YZfs),(Xf,Cd,YZft),(Xf,Cd,YZfu),(Xf,Cd,YZfv),(Xf,Cd,YZfw),(Xf,Cd,YZfx),(Xf,Cd, YZfy),(Xf,Cd,YZfz),(Xf,Cd,YZga),(Xf,Cd,YZgb),(Xf,Cd,YZgc),(Xf,Cd,YZgd),(Xf, Cd,YZge),(Xf,Cd,YZgf),(Xf,Cd,YZgg),(Xf,Cd,YZgh),(Xf,Cd,YZgi),(Xf,Cd,YZgj),(Xf, Cd,YZgk),(Xf,Cd,YZgl),(Xf,Cd,YZgm),(Xf,Cd,YZgn),(Xf,Cd,YZgo),(Xf,Cd,YZgp),(Xf, Cd,YZgq),(Xf,Cd,YZgr),(Xf,Cd,YZgs),(Xf,Cd,YZgt),(Xf,Ce,YZa),(Xf,Ce,YZb),(Xf, Ce,YZc),(Xf,Ce,YZd),(Xf,Ce,YZe),(Xf,Ce,YZf),(Xf,Ce,YZg),(Xf,Ce,YZh),(Xf,Ce,YZi), (Xf,Ce,YZj),(Xf,Ce,YZk),(Xf,Ce,YZl),(Xf,Ce,YZm),(Xf,Ce,YZn),(Xf,Ce,YZo),(Xf, Ce,YZp),(Xf,Ce,YZq),(Xf,Ce,YZr),(Xf,Ce,YZs),(Xf,Ce,YZt),(Xf,Ce,YZu),(Xf,Ce,YZv), (Xf,Ce,YZw),(Xf,Cc,YZx),(Xf,Ce,YZy),(Xf,Ce,YZz),(Xf,Ce,YZaa),(Xf,Ce,YZab)), (Xf,Ce,YZac),(Xf,Ce,YZad),(Xf,Ce,YZae),(Xf,Ce,YZaf),(Xf,Ce,YZag),(Xf,Ce,YZah), (Xf,Ce,YZai),(Xf,Ce,YZaj),(Xf,Ce,YZak),(Xf,Ce,YZal),(Xf,Ce,YZam),(Xf,Ce,YZan), (Xf,Ce,YZao),(Xf,Ce,YZap),(Xf,Ce,YZaq),(Xf,Ce,YZar),(Xf,Ce,YZas),(Xf,Ce,YZat), (Xf,Ce,YZau),(Xf,Ce,YZav),(Xf,Ce,YZaw),(Xf,Ce,YZax),(Xf,Ce,YZay),(Xf,Ce,YZaz), (Xf,Cc,YZba),(Xf,Ce,YZbb),(Xf,Ce,YZbc),(Xf,Ce,YZbd),(Xf,Ce,YZbe),(Xf,Ce,YZbf), (Xf,Ce,YZbg),(Xf,Ce,YZbh),(Xf,Ce,YZbi),(Xf,Ce,YZbj),(Xf,Ce,YZbk),(Xf,Ce,YZbl), (Xf,Ce,YZbm),(Xf,Ce,YZbn),(Xf,Ce,YZbo),(Xf,Ce,YZbp),(Xf,Ce,YZbq),(Xf,Ce,YZbr), (Xf,Ce,YZbs),(Xf,Ce,YZbt),(Xf,Ce,YZbu),(Xf,Ce,YZbv),(Xf,Ce,YZbw),(Xf,Ce, YZbx),(Xf,Ce,YZby),(Xf,Ce,YZbz),(Xf,Ce,YZca),(Xf,Ce,YZcb),(Xf,Ce,YZcc),(Xf,Ce, YZcd),(Xf,Ce,YZce),(Xf,Ce,YZcf),(Xf,Ce,YZcg),(Xf,Ce,YZch),(Xf,Ce,YZci),(Xf,Ce, YZcj),(Xf,Ce,YZck),(Xf,Ce,YZcl),(Xf,Ce,YZcm),(Xf,Ce,YZcn),(Xf,Ce,YZco),(Xf,Ce, YZcp),(Xf,Ce,YZcq),(Xf,Ce,YZcr),(Xf,Ce,YZcs),(Xf,Ce,YZct),(Xf,Ce,YZcu),(Xf,Ce, YZcv),(Xf,Ce,YZcw),(Xf,Ce,YZcx),(Xf,Ce,YZcy),(Xf,Ce,YZcz),(Xf,Ce,YZda),(Xf,Ce, YZdb),(Xf,Ce,YZdc),(Xf,Ce,YZdd),(Xf,Ce,YZde),(Xf,Ce,YZdf),(Xf,Ce,YZdg),(Xf, Ce,YZdh),(Xf,Ce,YZdi),(Xf,Ce,YZdj),(Xf,Ce,YZdk),(Xf,Ce,YZdl),(Xf,Ce,YZdm),(Xf, Ce,YZdn),(Xf,Ce,YZdo),(Xf,Ce,YZdp),(Xf,Ce,YZdq),(Xf,Ce,YZdr),(Xf,Ce,YZds),(Xf, Ce,Yzdt),(Xf,Ce,YZdu),(Xf,Ce,YZdv),(Xf,Ce,YZdw),(Xf,Ce,YZdx),(Xf,Ce,YZdy),(Xf, Ce,YZdz),(Xf,Ce,YZea),(Xf,Ce,YZeb),(Xf,Ce,YZec),(Xf,Ce,YZed),(Xf,Ce,YZee),(Xf, Ce,YZef),(Xf,Ce,YZeg),(Xf,Ce,YZeh),(Xf,Ce,YZei),(Xf,Ce,YZej),(Xf,Ce,YZek),(Xf, Ce,YZel),(Xf,Ce,YZem),(Xf,Ce,YZen),(Xf,Ce,YZeo),(Xf,Ce,YZep),(Xf,Ce,YZeq), (Xf,Ce,YZer),(Xf,Ce,YZes),(Xf,Ce,YZet),(Xf,Ce,YZeu),(Xf,Ce,YZev),(Xf,Ce,YZew), (Xf,Ce,YZex),(Xf,Ce,YZey),(Xf,Ce,YZez),(Xf,Ce,YZfa),(Xf,Ce,YZfb),(Xf,Ce,YZfc),(Xf, Ce,YZfd),(Xf,Ce,YZfe),(Xf,Ce,YZff),(Xf,Ce,YZfg),(Xf,Ce,YZfh),(Xf,Ce,YZfi),(Xf, Ce,YZfj),(Xf,Ce,YZfk),(Xf,Ce,YZfl),(Xf,Ce,YZfm),(Xf,Ce,YZfn),(Xf,Ce,YZfo),(Xf, Ce,YZfp),(Xf,Ce,YZfq),(Xf,Ce,YZfr),(Xf,Ce,YZfs),(Xf,Ce,YZft),(Xf,Ce,YZfu),(Xf,Ce, YZfv),(Xf,Ce,YZfw),(Xf,Ce,YZfx),(Xf,Ce,YZfy),(Xf,Ce,YZfz),(Xf,Ce,YZga),(Xf,Ce, YZgb),(Xf,Ce,YZgc),(Xf,Ce,YZgd),(Xf,Ce,YZge),(Xf,Ce,YZgf),(Xf,Ce,YZgg),(Xf, Ce,YZgh),(Xf,Ce,YZgi),(Xf,Ce,YZgj),(Xf,Ce,YZgk),(Xf,Ce,YZgl),(Xf,Ce,YZgm),(Xf, Ce,YZgn),(Xf,Ce,YZgo),(Xf,Ce,YZgp),(Xf,Ce,YZgq),(Xf,Ce,YZgr),(Xf,Ce,YZgs),(Xf, Ce,YZgt),(Xf,Cf,YZa),(Xf,Cf,YZb),(Xf,Cf,YZc),(Xf,Cf,YZd),(Xf,Cf,YZe),(Xf,Cf,YZf), (Xf,Cf,YZg),(Xf,Cf,YZh),(Xf,Cf,YZi),(Xf,Cf,YZj),(Xf,Cf,YZk),(Xf,Cf,YZl),(Xf,Cf, YZm),(Xf,Cf,YZn),(Xf,Cf,YZo),(Xf,Cf,YZp),(Xf,Cf,YZq),(Xf,Cf,YZr),(Xf,Cf,YZs), (Xf,Cf,YZt),(Xf,Cf,YZu),(Xf,Cf,YZv),(Xf,Cf,YZw),(Xf,Cf,YZx),(Xf,Cf,YZy),(Xf,Cf, YZz),(Xf,Cf,YZaa),(Xf,Cf,YZab),(Xf,Cf,YZac),(Xf,Cf,YZad),(Xf,Cf,YZae),(Xf,Cf,YZaf), (Xf,Cf,YZag),(Xf,Cf,YZah),(Xf,Cf,YZai),(Xf,Cf,YZaj),(Xf,Cf,YZak),(Xf,Cf,YZal), (Xf,Cf,YZam),(Xf,Cf,YZan),(Xf,Cf,YZao),(Xf,Cf,YZap),(Xf,Cf,YZaq),(Xf,Cf,YZar), (Xf,Cf,YZas),(Xf,Cf,YZat),(Xf,Cf,YZau),(Xf,Cf,YZav),(Xf,Cf,YZaw),(Xf,Cf,YZax), (Xf,Cf,YZay),(Xf,Cf,YZaz),(Xf,Cf,YZba),(Xf,Cf,YZbb),(Xf,Cf,YZbc),(Xf,Cf,YZbd),(Xf, Cf,YZbe),(Xf,Cf,YZbf),(Xf,Cf,YZbg),(Xf,Cf,YZbh),(Xf,Cf,YZbi),(Xf,Cf,YZbj),(Xf, Cf,YZbk),(Xf,Cf,YZbl),(Xf,Cf,YZbm),(Xf,Cf,YZbn),(Xf,Cf,YZbo),(Xf,Cf,YZbp),(Xf, Cf,YZbq),(Xf,Cf,YZbr),(Xf,Cf,YZbs),(Xf,Cf,YZbt),(Xf,Cf,YZbu),(Xf,Cf,YZbv),(Xf, Cf,YZbw),(Xf,Cf,YZbx),(Xf,Cf,YZby),(Xf,Cf,YZbz),(Xf,Cf,YZca),(Xf,Cf,YZcb),(Xf, Cf,YZcc),(Xf,Cf,YZcd),(Xf,Cf,YZee),(Xf,Cf,YZcf),(Xf,Cf,YZcg),(Xf,Cf,YZch),(Xf,Cf, YZei),(fXcf,fYZcj),(Xf,Cf,YZck),(XfCf,YZcl),(Xf,Cf,YZcm),(Xf,Cf,YZcn),(Xf,Cf, YZco),(Xf,Cf,YZcp),(Xf,Cf,YZcq),(Xf,Cf,YZcr),(Xf,Cf,YZcs),(Xf,Cf,YZct),(Xf,Cf,YZcu), (Xf,Cf,YZcv),(Xf,Cf,YZcw),(Xf,Cf,YZcx),(Xf,Cf,YZcy),(Xf,Cf,YZcz),(Xf,Cf,YZda), (Xf,Cf,YZdb),(Xf,Cf,YZdc),(Xf,Cf,YZdd),(Xf,Cf,YZde),(Xf,Cf,YZdf),(Xf,Cf,YZdg), (Xf,Cf,YZdh),(Xf,Cf,YZdi),(Xf,Cf,YZdj),(Xf,Cf,YZdk),(Xf,Cf,YZdl),(Xf,Cf,YZdm), (Xf,Cf,YZdn),(Xf,Cf,YZdo),(Xf,Cf,YZdp),(Xf,Cf,YZdq),(Xf,Cf,YZdr),(Xf,Cf,YZds), (Xf,Cf,Yzdt),(Xf,Cf,YZdu),(Xf,Cf,YZdv),(Xf,Cf,YZdw),(Xf,Cf,YZdx),(Xf,Cf,YZdy), (Xf,Cf,YZdz),(Xf,Cf,YZea),(Xf,Cf,YZeb),(Xf,Cf,YZec),(Xf,Cf,YZed),(Xf,Cf,YZee), (Xf,Cf,YZef),(Xf,Cf,YZeg),(Xf,Cf,YZeh),(Xf,Cf,YZei),(Xf,Cf,YZej),(Xf,Cf,YZek), (Xf,Cf,YZel),(Xf,Cf,YZem),(Xf,Cf,YZen),(Xf,Cf,YZeo),(Xf,Cf,YZep),(Xf,Cf,YZeq),(Xf, Cf,YZer),(Xf,Cf,YZes),(Xf,Cf,YZet),(Xf,Cf,YZeu),(Xf,Cf,YZev),(Xf,Cf,YZew),(Xf, Cf,YZex),(Xf,Cf,YZey),(Xf,Cf,YZez),(Xf,Cf,YZfa),(Xf,Cf,YZfb),(Xf,CfE,YZfc),(Xf, Cf,YZfd),(Xf,Cf,YZfe),(Xf,Cf,YZff),(Xf,Cf,YZfg),(Xf,Cf,YZfh),(Xf,Cf,YZfi),(Xf,Cf, YZfj),(Xf,Cf,YZfk),(Xf,Cf,YZfl),(Xf,Cf,YZfm),(Xf,Cf,YZfn),(Xf,Cf,YZfo),(Xf,Cf,YZfp), (Xf,Cf,YZfq),(Xf,Cf,YZfr),(Xf,Cf,YZfs),(Xf,Cf,YZft),(Xf,Cf,YZfu),(Xf,Cf,YZfv), (Xf,Cf,YZfw),(Xf,Cf,YZfx),(Xf,Cf,YZfy),(Xf,Cf,YZfz),(Xf,Cf,YZga),(Xf,Cf,YZgb),(Xf, Cf,YZgc),(fXcf,fYZgd),(Xf,Cf,YZge),(Xf,Cf,YZgf),(Xf,Cf,YZgg),(Xf,Cf,YZgh),(Xf, Cf,YZgi),(Xf,Cf,YZgj),(Xf,Cf,YZgk),(Xf,Cf,YZgl),(Xf,Cf,YZgm),(Xf,Cf,YZgn),(Xf, Cf,YZgo),(Xf,Cf,YZgp),(Xf,Cf,YZgq),(Xf,Cf,YZgr),(Xf,Cf,YZgs),(Xf,Cf,YZgt), (Xg,Ca,YZa),(Xg,Ca,YZb),(Xg,Ca,YZc),(Xg,Ca,YZd),(Xg,Ca,YZe),(Xg,Ca,YZf),(Xg, Ca,YZg),(Xg,Ca,YZh),(Xg,Ca,YZi),(Xg,Ca,YZj),(Xg,Ca,YZk),(Xg,Ca,YZl),(Xg,Ca,YZm), (Xg,Ca,YZn),(Xg,Ca,YZo),(Xg,Ca,YZp),(Xg,Ca,YZq),(Xg,Ca,YZr),(Xg,Ca,YZs), (Xg,Ca,YZt),(Xg,Ca,YZu),(Xg,Ca,YZv),(Xg,Ca,YZw),(Xg,Ca,YZx),(Xg,Ca,YZy),(Xg, Ca,YZz),(Xg,Ca,YZaa),(Xg,Ca,YZab),(Xg,Ca,YZac),(Xg,Ca,YZad),(Xg,Ca,YZae),(Xg, Ca,YZaf),(Xg,Ca,YZag),(Xg,Ca,YZah),(Xg,Ca,YZai),(Xg,Ca,YZaj),(Xg,Ca,YZak), (Xg,Ca,YZal),(Xg,Ca,YZam),(Xg,Ca,Yan),(Xg,Ca,YZao),(Xg,Ca,YZap),(Xg,Ca,YZaq), (Xg,Ca,YZar),(Xg,Ca,YZas),(Xg,Ca,YZat),(Xg,Ca,YZau),(Xg,Ca,YZav),(Xg,Ca, YZaw),(Xg,Ca,YZax),(Xg,Ca,YZay),(Xg,Ca,YZaz),(Xg,Ca,YZba),(Xg,Ca,YZbb),(Xg, Ca,YZbc),(Xg,Ca,YZbd),(Xg,Ca,YZbe),(Xg,Ca,YZbf),(Xg,Ca,YZbg),(Xg,Ca,YZbb),(Xg, Ca,YZbi),(Xg,Ca,YZbj),(Xg,Ca,YZbk),(Xg,Ca,YZbl),(Xg,Ca,YZbm),(Xg,Ca,Yibn), (Xg,Ca,YZbo),(Xg,Ch,YZbp),(Xg,Ca,YZbq),(Xg,Ca,YZbr),(Xg,Ca,YZbs),(Xg,Ca, YZbt),(Xg,Ca,YZbu),(Xg,Ca,YZbv),(Xg,Ca,YZbw),(Xg,Ca,YZbx),(Xg,Ca,YZby),(Xg, Ca,YZbz),(Xg,Ca,YZca),(Xg,Ca,YZcb),(Xg,Ca,YZcc),(Xg,Ca,YZcd),(Xg,Ca,YZce),(Xg, Ca,YZcf),(Xg,Ca,YZcg),(Xg,Ca,YZch),(Xg,Ca,YZci),(Xg,Ca,YZcj),(Xg,Ca,YZck), (Xg,Ca,YZcl),(Xg,Ca,YZcm),(Xg,Ca,YZcn),(Xg,Ca,YZco),(Xg,Ca,YZcp),(Xg,Ca,YZcq), (Xg,Ca,YZcr),(Xg,Ca,YZcs),(Xg,Ca,YZet),(Xg,Ca,YZcu),(Xg,Ca,YZcv),(Xg,Ca, YZcw),(Xg,Ca,YZcx),(Xg,Ca,YZcy),(Xg,Ca,YZcz),(Xg,Ca,YZda),(Xg,Ca,YZdb),(Xg, Ca,YZdc),(Xg,Ca,YZdd),(Xg,Ca,YZde),(Xg,Ca,YZdf),(Xg,Ca,YZdg),(Xg,Ca,YZdh),(Xg, Ca,YZdi),(Xg,Ca,YZdj),(Xg,Ca,YZdk),(Xg,Ca,YZdl),(Xg,Ca,YZdm),(Xg,Ca,YZdn), (Xg,Ca,YZdo),(Xg,Ca,YZdp),(Xg,Ca,YZdq),(Xg,Ca,YZdr),(Xg,Ca,YZds),(Xg,Ca, Yzdt),(Xg,Ca,YZdu),(Xg,Ca,YZdv),(Xg,Ca,YZdw),(Xg,Ca,YZdx),(Xg,Ca,YZdy),(Xg, Ca,YZdz),(Xg,Ca,YZea),(Xg,Ca,YZeb),(Xg,Ca,YZec),(Xg,Ca,YZed),(Xg,Ca,YZee),(Xg, Ca,YZef),(Xg,Ca,YZeg),(Xg,Ca,YZeh),(Xg,Ca,YZei),(Xg,Ca,YZej),(Xg,Ca,YZek), (Xg,Ca,YZel),(Xg,Ca,YZem),(Xg,Ca,YZen),(Xg,Ca,YZeo),(Xg,Ca,YZep),(Xg,Ca,YZeq), (Xg,Ca,YZer),(Xg,Ca,YZes),(Xg,Ca,YZet),(Xg,Ca,YZeu),(Xg,Ca,YZev),(Xg,Ca, YZew),(Xg,Ca,YZex),(Xg,Ca,YZey),(Xg,Ca,YZez),(Xg,Ca,YZfa),(Xg,Ca,YZfb),(Xg, Ca,YZfc),(Xg,Ca,YZfd),(Xg,Ca,YZfe),(Xg,Ca,YZff),(Xg,Ca,YZfg),(Xg,Ca,YZfh),(Xg, Ca,YZfi),(Xg,Ca,YZfj),(Xg,Ca,YZfk),(Xg,Ca,YZfl),(Xg,Ca,YZfm),(Xg,Ca,YZfn),(Xg, Ca,YZfo),(Xg,Ca,YZfp),(Xg,Ca,YZfq),(Xg,Ca,YZfr),(Xg,Ca,YZfs),(Xg,Ca,YZft),(Xg, Ca,YZfu),(Xg,Ca,YZfv),(Xg,Ca,YZfw),(Xg,Ca,YZfx),(Xg,Ca,YZfy),(Xg,Ca,YZfz),(Xg, Ca,YZga),(Xg,Ca,YZgb),(Xg,Ca,YZgc),(Xg,Ca,YZgd),(Xg,Ca,YZge),(Xg,Ca,YZgf), (Xg,Ca,YZgg),(Xg,Ca,YZgh),(Xg,Ca,YZgi),(Xg,Ca,YZgj),(Xg,Ca,YZgk),(Xg,Ca,YZgl), (Xg,Ca,YZgm),(Xg,Ca,YZgn),(Xg,Ca,YZgo),(Xg,Ca,YZgp),(Xg,Ca,YZgq),(Xg, Ca,YZgr),(Xg,Ca,YZgs),(Xg,Ca,YZgt),(Xg,Cb,YZa),(Xg,Cb,YZb),(Xg,Cb,YZc),(Xg, Cb,YZd),(Xg,Cb,YZe),(Xg,Cb,YZf),(Xg,Cb,YZg),(Xg,Cb,YZh),(Xg,Cb,YZi),(Xg,Cb, YZj),(Xg,Cb,YZk),(Xg,Cb,YZl),(Xg,Cb,YZm),(Xg,Cb,YZn),(Xg,Cb,YZo),(Xg,Cb,YZp), (Xg,Cb,YZq),(Xg,Cb,YZr),(Xg,Cb,YZs),(Xg,Cb,YZt),(Xg,Cb,YZu),(Xg,Cb,YZv),(Xg, Cb,YZw),(Xg,Cb,YZx),(Xg,Cb,YZy),(Xg,Cb,YZz),(Xg,Cb,YZaa),(Xg,Cb,YZab),(Xg, Cb,YZac),(Xg,Cb,YZad),(Xg,Cb,YZae),(Xg,Cb,YZaf),(Xg,Cb,YZag),(Xg,Cb,YZah), (Xg,Cb,YZai),(Xg,Cb,YZaj),(Xg,Cb,YZak),(Xg,Cb,YZal),(Xg,Cb,YZam),(Xg,Cb, YZan),(Xg,Cb,YZao),(Xg,Cb,YZap),(Xg,Cb,YZaq),(Xg,Cb,YZar),(Xg,Cb,YZas),(Xg, Cb,YZat),(Xg,Cb,YZau),(Xg,Cb,YZav),(Xg,Cb,YZaw),(Xg,Qb,YZax),(Xg,Cb,YZay), (Xg,Cb,YZaz),(Xg,Cb,YZba),(Xg,Cb,YZbb),(Xg,Cb,YZbc),(Xg,Cb,YZbd),(Xg,Cb,YZbe), (Xg,Cb,YZbf),(Xg,Cb,YZbg),(Xg,Cb,YZbh),(Xg,Cb,YZbi),(Xg,Cb,YZbj),(Xg,Cb, YZbk),(Xg,Cb,YZbl),(Xg,Cb,YZbm),(Xg,Cb,YZbn),(Xg,Cb,YZbo),(Xg,Cb,YZbp),(Xg, Cb,YZbq),(Xg,Cb,YZbr),(Xg,Cb,YZbs),(Xg,Cb,YZbt),(Xg,Cb,YZbu),(Xg,Cb,YZbv), (Xg,Cb,YZbw),(Xg,Cb,YZbx),(Xg,Cb,YZby),(Xg,Cb,YZbz),(Xg,Cb,YZca),(Xg,Cb, YZeb),(Xg,Cb,YZcc),(Xg,Cb,YZcd),(Xg,Cb,YZce),(Xg,Cb,YZcf),(Xg,Cb,YZcg),(Xg, Cb,YZch),(Xg,Cb,YZci),(Xg,Cb,YZcj),(Xg,Cb,YZek),(Xg,Cb,YZcl),(Xg,Cb,YZcm),(Xg, Cb,YZcn),(Xg,Cb,YZco),(Xg,Cb,YZcp),(Xg,Cb,YZcq),(Xg,Cb,YZcr),(Xg,Cb,YZcs), (Xg,Cb,YZct),(Xg,Cb,YZcu),(Xg,Cb,YZcv),(Xg,Cb,YZcw),(Xg,Cb,YZcx),(Xg,Cb, YZcy),(Xg,Cb,YZcz),(Xg,Cb,YZda),(Xg,Cb,YZdb),(Xg,Cb,YZdc),(Xg,Cb,YZdd),(Xg, Cb,YZde),(Xg,Cb,YZdf),(Xg,Cb,YZdg),(Xg,Cb,YZdh),(Xg,Cb,YZdi),(Xg,Cb,YZdj),(Xg, Cb,YZdk),(Xg,Cb,YZdl),(Xg,Cb,YZdm),(Xg,Cb,YZdn),(Xg,Cb,YZdo),(Xg,Cb,YZdp), (Xg,Cb,YZdq),(Xg,Cb,YZdr),(Xg,Cb,YZds),(Xg,Cb,Yzdt),(Xg,Cb,YZdu),(Xg,Cb, YZdv),(Xg,Cb,YZdw),(Xg,Cb,YZdx),(Xg,Cb,YZdy),(Xg,Cb,YZdz),(Xg,Cb,YZea),(Xg, Cb,YZeb),(Xg,Cb,YZec),(Xg,Cb,YZed),(Xg,Cb,YZee),(Xg,Cb,YZef),(Xg,Cb,YZeg), (Xg,Cb,YZeh),(Xg,Cb,YZei),(Xg,Cb,YZej),(Xg,Cb,YZek),(Xg,Cb,YZel),(Xg,Cb,YZem), (Xg,Cb,YZen),(Xg,Cb,YZeo),(Xg,Cb,YZep),(Xg,Cb,YZeq),(Xg,Cb,YZer),(Xg,Cb, YZes),(Xg,Cb,YZet),(Xg,Cb,YZeu),(Xg,Cb,YZev),(Xg,Cb,YZew),(Xg,Cb,YZex),(Xg, Cb,YZey),(Xg,Cb,YZez),(Xg,Cb,YZfa),(Xg,Cb,YZfb),(Xg,Cb,YZfc),(Xg,Cb,YZfd),(Xg, Cb,YZfe),(Xg,Cb,YZff),(Xg,Cb,YZfg),(Xg,Cb,YZfh),(Xg,Cb,YZfi),(Xg,Cb,YZfj), (Xg,Cb,YZfk),(Xg,Cb,YZfl),(Xg,Cb,YZfm),(Xg,Cb,YZfn),(Xg,Cb,YZfo),(Xg,Cb,YZfp), (Xg,Cb,YZfq),(Xg,Cb,YZfr),(Xg,Cb,YZfs),(Xg,Cb,YZft),(Xg,Cb,YZfu),(Xg,Cb,YZfv), (Xg,Cb,YZfw),(Xg,Cb,YZfx),(Xg,Cb,YZfy),(Xg,Cb,YZfz),(Xg,Cb,YZga),(Xg,Cb, YZgb),(Xg,Cb,YZgc),(Xg,Cb,YZgd),(Xg,Cb,YZge),(Xg,Cb,YZgf),(Xg,Cb,YZgg),(Xg, Cb,YZgh),(Xg,Cb,YZgi),(Xg,Cb,YZgj),(Xg,Cb,YZgk),(Xg,Cb,YZgl),(Xg,Cb,YZgm), (Xg,Cb,YZgn),(Xg,Cb,YZgo),(Xg,Cb,YZgp),(Xg,Cb,YZgq),(Xg,Cb,YZgr),(Xg,Cb,YZgs), (Xg,Cb,YZgt),(Xg,Cc,YZa),(Xg,Cc,YZb),(Xg,Cc,YZc),(Xg,Cc,YZd),(Xg,Cc,YZe), (Xg,Cc,YZf),(Xg,Cc,YZg),(Xg,Cc,YZh),(Xg,Cc,YZi),(Xg,Cc,YZj),(Xg,Cc,YZk),(Xg, Cc,YZl),(Xg,Cc,YZm),(Xg,Cc,YZn),(Xg,Cc,YZo),(Xg,Cc,YZp),(Xg,Cc,YZq),(Xg,Cc, YZr),(Xg,Cc,YZs),(Xg,Cc,YZt),(Xg,Cc,YZu),(Xg,Cc,YZv),(Xg,Cc,YZw),(Xg,Cc,YZx), (Xg,Cc,YZy),(Xg,Cc,YZz),(Xg,Cc,YZaa),(Xg,Cc,YZab),(Xg,Cc,YZac),(Xg,Cc,YZad), (Xg,Cc,YZae),(Xg,Cc,YZaf),(Xg,Cc,YZag),(Xg,Cc,YZah),(Xg,Cc,YZai),(Xg,Cc, YZaj),(Xg,Cc,YZak),(Xg,Cc,YZal),(Xg,Cc,YZam),(Xg,Cc,YZan),(Xg,Cc,YZao),(Xg, Cc,YZap),(Xg,Cc,YZaq),(Xg,Cc,YZar),(Xg,Cc,YZas),(Xg,Cc,YZat),(Xg,Cc,YZau),(Xg, Cc,YZav),(Xg,Cc,YZaw),(Xg,Cc,YZax),(Xg,Cc,YZay),(Xg,Cc,YZaz),(Xg,Cc,YZba), (Xg,Cc,YZbb),(Xg,Cc,YZbc),(Xg,Cc,YZbd),(Xg,Cc,YZbe),(Xg,Cc,YZbf),(Xg,Cc,YZbg), (Xg,Cc,YZbh),(Xg,Cc,YZbi),(Xg,Cc,YZbj),(Xg,Cc,YZbk),(Xg,Cc,YZbl),(Xg,Cc, YZbm),(Xg,Cc,YZbn),(Xg,Cc,YZbo),(Xg,Cc,YZbp),(Xg,Cc,YZbq),(Xg,CG,YZbr),(Xg, Cc,YZbs),(Xg,Cc,YZbt),(Xg,Cc,YZbu),(Xg,Cc,YZbv),(Xg,Cc,YZbw),(Xg,Cc,YZbx), (Xg,Cc,YZby),(Xg,Cc,YZbz),(Xg,Cc,YZca),(Xg,Cc,YZcb),(Xg,Cc,YZcc),(Xg,Cc,YZcd), (Xg,Cc,YZce),(Xg,Cc,YZcf),(Xg,Cc,YZcg),(Xg,Cc,YZch),(Xg,Cc,YZei),(Xg,Cc, YZcj),(Xg,Cc,YZck),(Xg,Cc,YZcl),(Xg,Cc,YZcm),(Xg,Cc,YZcn),(Xg,Cc,YZco),(Xg, Cc,YZcp),(Xg,Cc,YZcq),(Xg,Cc,YZcr),(Xg,Cc,YZcs),(Xg,Cc,YZct),(Xg,Cc,YZcu),(Xg, Cc,YZcv),(Xg,Cc,YZcw),(Xg,Cc,YZcx),(Xg,Cc,YZcy),(Xg,Cc,YZcz),(Xg,Cc,YZda), (Xg,Cc,YZdb),(Xg,Cc,YZdc),(Xg,Cc,YZdd),(Xg,Cc,YZde),(Xg,Cc,YZdf),(Xg,Cc,YZdg), (Xg,Cc,YZdh),(Xg,Cc,YZdi),(Xg,Cc,YZdj),(Xg,Cc,YZdk),(Xg,Cc,YZdl),(Xg,Cc, YZdm),(Xg,Cc,YZdn),(Xg,Cc,YZdo),(Xg,Cc,YZdp),(Xg,Cc,YZdq),(Xg,Cc,YZdr),(Xg, Cc,YZds),(Xg,Cc,Yzdt),(Xg,Cc,YZdu),(Xg,Cc,YZdv),(Xg,Cc,YZdw),(Xg,Cc,YZdx), (Xg,Cc,YZdy),(Xg,Cc,YZdz),(Xg,Cc,YZea),(Xg,Cc,YZeb),(Xg,Cc,YZec),(Xg,Cc,YZed), (Xg,Cc,YZee),(Xg,Cc,YZef),(Xg,Cc,YZeg),(Xg,Cc,YZeh),(Xg,Cc,YZei),(Xg,Cc, YZej),(Xg,Cc,YZek),(Xg,Cc,YZel),(Xg,Cc,YZem),(Xg,Cc,YZen),(Xg,Cc,YZeo),(Xg, Cc,YZep),(Xg,Cc,YZeq),(Xg,Cc,YZer),(Xg,Cc,YZes),(Xg,Cc,YZet),(Xg,Cc,YZeu),(Xg, Cc,YZev),(Xg,Cc,YZew),(Xg,Cc,YZex),(Xg,Cc,YZey),(Xg,Cr,YZez),(Xg,Cc,YZfa), (Xg,Cc,YZfb),(Xg,Cc,YZfc),(Xg,Cc,YZfd),(Xg,Cc,YZfe),(Xg,Cc,YZff),(Xg,Cc,YZfg), (Xg,Cc,YZfh),(Xg,Cc,YZfi),(Xg,Cc,YZij),(Xg,Cc,YZfk),(Xg,Cc,YZfl),(Xg,Cc,YZfm), (Xg,Cc,YZfn),(Xg,Cc,YZfo),(Xg,Cc,YZfp),(Xg,Cc,YZfq),(Xg,Cc,YZfr),(Xg,Cc,YZfs), (Xg,Cc,YZft),(Xg,Cc,YZfu),(Xg,Cc,YZfv),(Xg,Cc,YZfw),(Xg,Cc,YZfx),(Xg,Cc, YZfy),(Xg,Cc,YZfz),(Xg,Cc,YZga),(Xg,Cc,YZgb),(Xg,Cc,YZgc),(Xg,Cc,YZgd),(Xg, Cc,YZge),(Xg,Cc,YZgf),(Xg,Cc,YZgg),(Xg,Cc,YZgh),(Xg,Cc,YZgi),(Xg,Cc,YZgj),(Xg, Cc,YZgk),(Xg,Cc,YZgl),(Xg,Cc,YZgm),(Xg,Cc,YZgn),(Xg,Cc,YZgo),(Xg,Cc,YZgp), (Xg,Cc,YZgq),(Xg,Cc,YZgr),(Xg,Cc,YZgs),(Xg,Cc,YZgt),(Xg,Cd,YZa),(Xg,Cd,YZb), (Xg,Cd,YZc),(Xg,Cd,YZd),(Xg,Cd,YZe),(Xg,Cd,YZf),(Xg,Cd,YZg),(Xg,Cd,YZh), (Xg,Cd,YZi),(Xg,Cd,YZj),(Xg,Cd,YZk),(Xg,Cd,YZl),(Xg,Cd,YZm),(Xg,Cd,YZn),(Xg, Cd,YZo),(Xg,Cd,YZp),(Xg,Cd,YZq),(Xg,Cd,YZr),(Xg,Cd,YZs),(Xg,Cd,YZt),(Xg,Cd, YZu),(Xg,Cd,YZv),(Xg,Cd,YZw),(Xg,Cd,YZx),(Xg,Cd,YZy),(Xg,Cd,YZz),(Xg,Cd, YZaa),(Xg,Cd,YZab),(Xg,Cd,YZac),(Xg,Cd,YZad),(Xg,Cd,YZae),(Xg,Cd,YZaf),(Xg, Cd,YZag),(Xg,Cd,YZah),(Xg,Cd,YZai),(Xg,Cd,YZaj),(Xg,Cd,YZak),(Xg,Cd,YZal),(Xg, Cd,YZam),(Xg,Cd,YZan),(Xg,Cd,YZao),(Xg,Cd,YZap),(Xg,Cd,YZaq),(Xg,Cd,YZar), (Xg,Cd,YZas),(Xg,Cd,YZat),(Xg,Cd,YZau),(Xg,Cd,YZav),(Xg,Cd,YZaw),(Xg,Cd, YZax),(Xg,Cd,YZay),(Xg,Cd,YZaz),(Xg,Cd,YZba),(Xg,Cd,YZbb),(Xg,Cd,YZbc),(Xg, Cd,YZbd),(Xg,Cd,YZbe),(Xg,Cd,YZbf),(Xg,Cd,YZbg),(Xg,Cd,YZbh),(Xg,Cd,YZbi), (Xg,Cd,YZbj),(Xg,Cd,YZbk),(Xg,Cd,YZbl),(Xg,Cd,YZbm),(Xg,Cd,YZbn),(Xg,Cd,YZbo), (Xg,Cd,YZbp),(Xg,Cd,YZbq),(Xg,Cd,YZbr),(Xg,Cd,YZbs),(Xg,Cd,YZbt),(Xg,Cd, YZbu),(Xg,Cd,YZbv),(Xg,Cd,YZbw),(Xg,Cd,YZbx),(Xg,Cd,YZby),(Xg,Cd,YZbz),(Xg, Cd,YZca),(Xg,Cd,YZcb),(Xg,Cd,YZcc),(Xg,Cd,YZcd),(Xg,Cd,YZce),(Xg,Cd,YZcf), (Xg,Cd,YZcg),(Xg,Cd,YZch),(Xg,Cd,YZci),(Xg,Cd,YZcj),(Xg,Cd,YZck),(Xg,Cd,YZcl), (Xg,Cd,YZcm),(Xg,Cd,YZcn),(Xg,Cd,YZco),(Xg,Cd,YZcp),(Xg,Cd,YZcq),(Xg, Cd,YZcr),(Xg,Cd,YZcs),(Xg,Cd,YZct),(Xg,Cd,YZcu),(Xg,Cd,YZcv),(Xg,Cd,YZcw),(Xg, Cd,YZcx),(Xg,Cd,YZcy),(Xg,Cd,YZcz),(Xg,Cd,YZda),(Xg,Cd,YZdb),(Xg,Cd,YZdc), (Xg,Cd,YZdd),(Xg,Cd,YZde),(Xg,Cd,YZdf),(Xg,Cd,YZdg),(Xg,Cd,YZdh),(Xg,Cd, YZdi),(Xg,Cd,YZdj),(Xg,Cd,YZdk),(Xg,Cd,YZdl),(Xg,Cd,YZdm),(Xg,Cd,YZdn),(Xg, Cd,YZdo),(Xg,Cd,YZdp),(Xg,Cd,YZdq),(Xg,Cd,YZdr),(Xg,Cd,YZds),(Xg,Cd,Yzdt), (Xg,Cd,YZdu),(Xg,Cd,YZdv),(Xg,Cd,YZdw),(Xg,Cd,YZdx),(Xg,Cd,YZdy),(Xg,Cd, YZdz),(Xg,Cd,YZea),(Xg,Cd,YZeb),(Xg,Cd,YZec),(Xg,Cd,YZed),(Xg,Cd,YZee),(Xg, Cd,YZef),(Xg,Cd,YZeg),(Xg,Cd,YZeh),(Xg,Cd,YZei),(Xg,Cd,YZej),(Xg,Cd,YZek),(Xg, Cd,YZel),(Xg,Cd,YZem),(Xg,Cd,YZen),(Xg,Cd,YZeo),(Xg,Cd,YZep),(Xg,Cd,YZeq), (Xg,Cd,YZer),(Xg,Cd,YZes),(Xg,Cd,YZet),(Xg,Cd,YZeu),(Xg,Cd,YZev),(Xg,Cd, YZew),(Xg,Cd,YZex),(Xg,Cd,YZey),(Xg,Cd,YZez),(Xg,Cd,YZfa),(Xg,Cd,YZfb),(Xg, Cd,YZfe),(Xg,Cd,YZfd),(Xg,Cd,YZfe),(Xg,Cd,YZff),(Xg,Cd,YZfg),(Xg,Cd,YZfh),(Xg, Cd,YZfi),(Xg,Cd,YZfj),(Xg,Cd,YZfk),(Xg,Cd,YZfl),(Xg,Cd,YZfm),(Xg,Cd,YZft),(Xg, Cd,YZfo),(Xg,Cd,YZfp),(Xg,Cd,YZfq),(Xg,Cd,YZfr),(Xg,Cd,YZfs),(Xg,Cd,YZft), (Xg,Cd,YZfu),(Xg,Cd,YZfv),(Xg,Cd,YZfw),(Xg,Cd,YZfx),(Xg,Cd,YZfy),(Xg,Cd,YZfz), (Xg,Cd,YZga),(Xg,Cd,YZgb),(Xg,Cd,YZgc),(Xg,Cd,YZgd),(Xg,Cd,YZge),(Xg,Cd, YZgf),(Xg,Cd,YZgg),(Xg,Cd,YZgh),(Xg,Cd,YZgi),(Xg,Cd,YZgj),(Xg,Cd,YZgk),(Xg, Cd,YZgl),(Xg,Cd,YZgm),(Xg,Cd,YZgn),(Xg,Cd,YZgo),(Xg,Cd,YZgp),(Xg,Cd,YZgq), (Xg,Cd,YZgr),(Xg,Cd,YZgs),(Xg,Cd,YZgt),(Xg,Ce,YZa),(Xg,Ce,YZb),(Xg,Ce,YZc), (Xg,Ce,YZd),(Xg,Ce,YZe),(Xg,Ce,YZf),(Xg,Ce,YZg),(Xg,Ce,YZh),(Xg,Ce,YZi),(Xg, Ce,YZj),(Xg,Ce,YZk),(Xg,Ce,YZl),(Xg,Ce,YZm),(Xg,Ce,YZn),(Xg,Ce,YZo),(Xg,Ce, YZp),(Xg,Ce,YZq),(Xg,Ce,YZr),(Xg,Ce,YZs),(Xg,Ce,YZt),(Xg,Ce,YZu),(Xg,Ce,YZv), (Xg,Ce,YZw),(Xg,Ce,YZx),(Xg,Ce,YZy),(Xg,Ce,YZz),(Xg,Ce,YZaa),(Xg,Ce,YZab), (Xg,Ce,YZac),(Xg,Ce,YZad),(Xg,Ce,YZae),(Xg,Ce,YZaf),(Xg,Ce,YZag),(Xg,Ce,YZah), (Xg,Ce,YZai),(Xg,Ce,YZaj),(Xg,Ce,YZak),(Xg,Ce,YZal),(Xg,Ce,YZam),(Xg,Ce, YZan),(Xg,Ce,YZao),(Xg,Ce,YZap),(Xg,Ce,YZaq),(Xg,Ce,YZar),(Xg,Ce,YZas),(Xg, Ce,YZat),(Xg,Ce,YZau),(Xg,Ce,YZav),(Xg,Ce,YZaw),(Xg,Ce,YZax),(Xg,Ce,YZay),(Xg, Ce,YZaz),(Xg,Ce,YZba),(Xg,Ce,YZbb),(Xg,Ce,YZbc),(Xg,Ce,YZbd),(Xg,Ce,YZbe), (Xg,Ce,YZbf),(Xg,Ce,YZbg),(Xg,Ce,YZbh),(Xg,Ce,YZbi),(Xg,Ce,YZbj),(Xg,Ce,YZbk), (Xg,Ce,YZbl),(Xg,Ce,YZbm),(Xg,Ce,YZbn),(Xg,Ce,YZbo),(Xg,Ce,YZbp),(Xg, Ce,YZbq),(Xg,Ce,YZbr),(Xg,Ce,YZbs),(Xg,Ce,YZbt),(Xg,Ce,YZbu),(Xg,Ce,YZbv),(Xg, Ce,YZbw),(Xg,Ce,nYZbx),(Xg,Ce,YZby),(Xg,Ce,YZbz),(Xg,Ce,YZca),(Xg,Ce,YZcb), (Xg,Ce,YZcc),(Xg,Ce,YZcd),(Xg,Ce,YZce),(Xg,Ce,YZcf),(Xg,Ce,YZcg),(Xg,Ce, YZch),(Xg,Ce,YZci),(Xg,Ce,YZcj),(Xg,Ce,YZck),(Xg,Ce,YZcl),(Xg,Ce,YZcm),(Xg, Ce,YZcn),(Xg,Ce,YZco),(Xg,Ce,YZcp),(Xg,Ce,YZcq),(Xg,Ce,YZcr),(Xg,Ce,YZcs),(Xg, Ce,YZct),(Xg,Ce,YZcu),(Xg,Ce,YZcv),(Xg,Ce,YZcw),(Xg,Ce,YZcx),(Xg,Ce,YZey), (Xg,Ce,YZcz),(Xg,Ce,YZda),(Xg,Ce,YZdb),(Xg,Ce,YZdc),(Xg,Ce,YZdd),(Xg,Ce, YZde),(Xg,Ce,YZdf),(Xg,Ce,YZdg),(Xg,Ce,YZdh),(Xg,Ce,YZdi),(Xg,Ce,YZdj),(Xg, Ce,YZdk),(Xg,Ce,YZdl),(Xg,Ce,YZdm),(Xg,Ce,YZdn),(Xg,Ce,YZdo),(Xg,Ce,YZdp); (Xg,Ce,YZdq),(Xg,Ce,YZdr),(Xg,Ce,YZds),(Xg,Ce,Yzdt),(Xg,Ce,YZdu),(Xg,Ce,YZdv), (Xg,Ce,YZdw),(Xg,Ce,YZdx),(Xg,Ce,YZdy),(Xg,Ce,YZdz),(Xg,Ce,YZea),(Xg,Ce, YZeb),(Xg,Ce,YZec),(Xg,Ce,YZed),(Xg,Ce,YZee),(Xg,Ce,YZef),(Xg,Ce,YZeg),(Xg, Ce,YZeh),(Xg,Ce,YZei),(Xg,Ce,YZej),(Xg,Ce,YZek),(Xg,Ce,YZel),(Xg,Ce,YZem),(Xg, Ce,YZen),(Xg,Ce,YZeo),(Xg,Ce,YZep),(Xg,Ce,YZeq),(Xg,Ce,YZer),(Xg,Ce,YZes), (Xg,Ce,YZet),(Xg,Ce,YZeu),(Xg,Ce,YZev),(Xg,Ce,YZew),(Xg,Ce,YZex),(Xg,Ce,YZey), (Xg,Ce,YZez),(Xg,Ce,YZfa),(Xg,Ce,YZfb),(Xg,Ce,YZfc),(Xg,Ce,YZfd),(Xg,Ce, YZfe),(Xg,Ce,YZff),(Xg,Ce,YZfg),(Xg,Ce,YZfh),(Xg,Ce,YZfi),(Xg,Ce,YZfj),(Xg,Ce, YZfk),(Xg,Ce,YZfl),(Xg,Ce,YZfm),(Xg,Ce,YZfm),(Xg,Ce,YZfo),(Xg,Ce,YZfp),(Xg,Ce, YZfq),(Xg,Ce,YZfr),(Xg,Ce,YZfs),(Xg,Ce,YZft),(Xg,Ce,YZfu),(Xg,Ce,YZfv),(Xg,Ce, YZfw),(Xg,Ce,YZfx),(Xg,Ce,YZfy),(Xg,Ce,YZfz),(Xg,Ce,YZga),(Xg,Ce,YZgb),(Xg, Ce,YZgc),(Xg,Ce,YZgd),(Xg,Ce,YZge),(Xg,Ce,YZgf),(Xg,Ce,YZgg),(Xg,Ce,YZgh), (Xg,Ce,YZgi),(Xg,Ce,YZgj),(Xg,Ce,YZgk),(Xg,Ce,YZgl),(Xg,Ce,YZgm),(Xg,Ce,YZgn), (Xg,Ce,YZgo),(Xg,Ce,YZgp),(Xg,Ce,YZgq),(Xg,Ce,YZgr),(Xg,Ce,YZgs),(Xg,Ce, YZgt),(Xg,Cf,YZa),(Xg,Cf,YZb),(Xg,Cf,YZc),(Xg,Cf,YZd),(Xg,Cf,YZe),(Xg,Cf,YZf), (Xg,Cf,YZg),(Xg,Cf,YZh),(Xg,Cf,YZi),(Xg,Cf,YZj),(Xg,Cf,YZk),(Xg,Cf,YZl),(Xg,Cf, YZm),(Xg,Cf,YZn),(Xg,Cf,YZo),(Xg,Cf,YZp),(Xg,Cf,YZq),(Xg,Cf,YZr),(Xg,Cf,YZs), (Xg,Cf,YZt),(Xg,Cf,YZu),(Xg,Cf,YZv),(Xg,Cf,YZw),(Xg,Cf,YZx),(Xg,Cf,YZy),(Xg, Cf,YZz),(Xg,Cf,YZaa),(Xg,Cf,YZab),(Xg,Cf,YZac),(Xg,Cf,YZad),(Xg,Cf,YZae),(Xg, Cf,YZaf),(Xg,Cf,YZag),(Xg,Cf,YZah),(Xg,Cf,YZai),(Xg,Cf,YZaj),(Xg,Cf,YZak),(Xg, Cf,YZal),(Xg,Cf,YZam),(Xg,Cf,YZan),(Xg,Cf,YZao),(Xg,Cf,YZap),(Xg,Cf,YZaq),(Xg, Cf,YZar),(Xg,Cf,YZas),(Xg,Cf,YZat),(Xg,Cf,YZau),(Xg,Cf,YZav),(Xg,Cf,YZaw), (Xg,Cf,YZax),(Xg,Cf,YZay),(Xg,Cf,YZaz),(Xg,Cf,YZba),(Xg,Cf,YZbb),(Xg,Cf,YZbc), (Xg,Cf,YZbd),(Xg,Cf,YZbe),(Xg,Cf,YZbf),(Xg,Cf,YZbg),(Xg,Cf,YZbh),(Xg,Cf,YZbi), (Xg,Cf,YZbj),(Xg,Cf,YZbk),(Xg,Cf,YZbl),(Xg,Cf,YZbm),(Xg,Cf,YZbn),(Xg,Cf,YZbo), (Xg,Cf,YZbp),(Xg,Cf,YZbq),(Xg,Cf,YZbr),(Xg,Cf,YZbs),(Xg,Cf,YZbt),(Xg,Cf, YZbu),(Xg,Cf,YZbv),(Xg,Cf,YZbw),(Xg,Cf,YZbx),(Xg,Cf,YZby),(Xg,Cf,YZbz),(Xg, Cf,YZca),(Xg,Cf,YZcb),(Xg,Cf,YZcc),(Xg,Cf,YZcd),(Xg,Cf,YZce),(Xg,Cf,YZcf),(Xg, Cf,YZcg),(Xg,Cf,YZch),(Xg,Cf,YZci),(Xg,Cf,YZcj),(Xg,Cf,YZck),(Xg,Cf,YZcl),(Xg Cf,YZcm),(Xg,Cf,YZcn),(Xg,Cf,YZco),(Xg,Cf,YZcp),(Xg,Cf,YZcq),(Xg,Cf,YZcr),(Xg, Cf,YZcs),(Xg,Cf,YZct),(Xg,Cf,YZcu),(Xg,Cf,YZcv),(Xg,Cf,YZcw),(Xg,Cf,YZGx), (Xg,Cf,YZcy),(Xg,Cf,YZcz),(Xg,Cf,YZda),(Xg,Cf,YZdb),(Xg,Cf,YZdc),(Xg,Cf,YZdd), (Xg,Cf,YZde),(Xg,Cf,YZdf),(Xg,Cf,YZdg),(Xg,Cf,YZdh),(Xg,Cf,YZdi),(Xg,Cf,YZdj), (Xg,Cf,YZdk),(Xg,Cf,YZdl),(Xg,Cf,YZdm),(Xg,Cf,YZdn),(Xg,Cf,YZdo),(Xg,Cf, YZdp),(Xg,Cf,YZdq),(Xg,Cf,YZdr),(Xg,Cf,YZds),(Xg,Cf,Yzdt),(Xg,Cf,YZdu),(Xg,Cf, YZdv),(Xg,Cf,YZdw),(Xg,Cf,YZdx),(Xg,Cf,YZdy),(Xg,Cf,YZdz),(Xg,Cf,YZea),(Xg, Cf,YZeb),(Xg,Cf,YZec),(Xg,Cf,YZed),(Xg,Cf,YZee),(Xg,Cf,YZef),(Xg,Cf,YZeg),(Xg, Cf,YZeh),(Xg,Cf,YZei),(Xg,Cf,YZej),(Xg,Cf,YZek),(Xg,Cf,YZel),(Xg,Cf,YZem),(Xg, Cf,YZen),(Xg,Cf,YZeo),(Xg,Cf,YZep),(Xg,Cf,YZeq),(Xg,Cf,YZer),(Xg,Cf,YZes),(Xg, Cf,YZet),(Xg,Cf,YZeu),(Xg,Cf,YZev),(Xg,Cf,YZew),(Xg,Cf,YZex),(Xg,Cf,YZey), (Xg,Cf,YZez),(Xg,Cf,YZfa),(Xg,Cf,YZfb),(Xg,Cf,YZfc),(Xg,Cf,YZfd),(Xg,Cf,YZfe), (Xg,Cf,YZff),(Xg,Cf,YZfg),(Xg,Cf,YZfh),(Xg,Cf,YZfi),(Xg,Cf,YZfji),(Xg,Cf,YZfk),(Xg, Cf,YZfl),(Xg,Cf,YZfm),(Xg,Cf,YZfn),(Xg,Cf,YZfo),(Xg,Cf,YZfp),(Xg,Cf,YZfq),(Xg, Cf,YZfr),(Xg,Cf,YZfs),(Xg,Cf,YZft),(Xg,Cf,YZfu),(Xg,Cf,YZfv),(Xg,Cf,YZfw),(Xg, Cf,YZfx),(Xg,Cf,YZfy),(Xg,Cf,YZfz),(Xg,Cf,YZga),(Xg,Cf,YZgb),(Xg,Cf,YZgc), (Xg,Cf,YZgd),(Xg,Cf,YZge),(Xg,Cf,YZgf),(Xg,Cf,YZgg),(Xg,Cf,YZgh),(Xg,Cf,YZgi), (Xg,Cf,YZgj),(Xg,Cf,YZgk),(Xg,Cf,YZgl),(Xg,Cf,YZgm),(Xg,Cf,YZgn),(Xg,Cf,YZgo), (Xg,Cf,YZgp),(Xg,Cf,YZgq),(Xg,Cf,YZgr),(Xg,Cf,YZgs),(Xg,Cf,YZgt), (Xh,Ca,YZa),(Xh,Ca,YZb),(Xh,Ca,YZc),(Xh,Ca,YZd),(Xh,Ca,YZe),(Xh,Ca,YZf),(Xh, Ca,YZg),(Xh,Ca,YZh),(Xh,Ca,YZi),(Xh,Ca,YZj),(Xh,Ca,YZk),(Xh,Ca,YZl),(Xh,Ca,YZm), (Xh,Ca,YZn),(Xh,Ca,YZo),(Xh,Ca,YZp),(Xh,Ca,YZq),(Xh,Ca,YZr),(Xh,Ca,YZs), (Xh,Ca,YZt),(Xh,Ca,YZu),(Xh,Ca,YZv),(Xh,Ca,YZw),(Xh,Ca,YZx),(Xh,Ca,YZy),(Xh, Ca,YZz),(Xh,Ca,YZaa),(Xh,Ca,YZab),(Xh,Ca,YZac),(Xh,Ca,YZad),(Xh,Ca,YZae),(Xh, Ca,YZaf),(Xh,Ca,YZag),(YXh,Ca,YZah),(Xh,Ca,YZai),(Xh,Ca,YZaj),(Xh,Ca,YZak), (YXh,Ca,YZal),(Xh,Ca,YZam),(Xh,Ca,YZan),(YXh,Ca,YZao),(Xh,Ca,YZap),(Xh,Ca,YZaq), (Xh,Ca,YZar),(Xh,Ca,YZas),(Xh,Ca,YZat),(Xh,Ca,YZau),(Xh,Ca,YZav),(Xh,Ca, YZaw),(Xh,Ca,YZax),(Xh,Ca,YZay),(Xh,Ca,YZaz),(Xh,Ca,YZba),(Xh,Ca,YZbb),(Xh, Ca,YZbc),(Xh,Ca,YZbd),(Xh,Ca,YZbe),(Xh,Ca,YZbf),(Xh,Ca,YZbg),(Xh,Ca,YZbh),(Xh, Ca,YZbi),(Xh,Ca,YZbj),(Xh,Ca,YZbk),(Xh,Ca,YZbl),(Xh,Ca,YZbm),(Xh,Ca,YZbn), (Xh,Ca,YZbo),(Xh,Ca,YZbp),(Xh,Ca,YZbq),(Xh,Ca,YZbr),(Xh,Ca,YZbs),(Xh,Ca, YZbt),(Xh,Ca,YZbu),(Xh,Ca,YZbv),(YXh,Ca,YZbw),(Xh,Ca,YZbx),(Xb,Ca,YZby),(Xh, Ca,YZbz),(Xh,Ca,YZca),(Xh,Ca,YZcb),(Xh,Ca,YZcc),(Xh,Ca,YZcd),(Xh,Ca,YZee),(Xh, Ca,YZcf),(Xh,Ca,YZcg),(Xh,Ca,YZch),(Xh,Ca,YZci),(Xh,Ca,YZcj),(Xh,Ca,YZck), (Xh,Ca,YZcl),(XhA,Ca,YZcm),(Xh,Ca,YZcn),(Xh,Ca,YZco),(Xh,Ca,YZcp),(Xh,Ca,YZcq), (Xh,Ca,YZcr),(Xh,Ca,YZcs),(Xh,Ca,YZct),(Xh,Ca,YZcu),(Xh,Ca,YZcv),(Xh,Ca, YZew),(Xh,Ca,YZcx),(Xh,Ca,YZcy),(Xh,Ca,YZcz),(Xh,Ca,YZda),(Xh,Ca,YZdb),(Xh, Ca,YZdc),(Xh,Ca,YZdd),(Xh,Ca,YZde),(Xh,Ca,YZdf),(Xh,Ca,YZdg),(Xh,Ca,YZdh),(Xh, Ca,YZdi),(Xh,Ca,YZdj),(Xh,Ca,YZdk),(Xh,Ca,YZdl),(Xh,Ca,YZdm),(Xh,Ca,YZdn), (Xh,Ca,YZdo),(Xh,Ca,YZdp),(Xh,Ca,YZdq),(Xh,Ca,YZdr),(Xh,Ca,YZds),(Xh,Ca, Yzdt),(Xh,Ca,YZdu),(Xh,Ca,YZdv),(Xh,Ca,YZdw),(Xh,Ca,YZdx),(Xh,Ca,YZdy),(Xh, Ca,YZdz),(Xh,Ca,YZea),(Xh,Ca,YZeb),(Xh,Ca,YZec),(Xh,Ca,YZed),(Xh,Ca,YZee),(Xh, Ca,YZef),(Xh,Ca,YZeg),(Xh,Ca,YZeh),(Xh,Ca,YZei),(Xh,Ca,YZej),(Xh,Ca,YZek), (Xh,Ca,YZel),(Xh,Ca,YZem),(Xh,Ca,YZen),(Xh,Ca,YZeo),(Xh,Ca,YZep),(Xh,Ca,YZeq), (Xh,Ca,YZer),(Xh,Ca,YZes),(Xh,Ca,YZet),(Xh,Ca,YZeu),(Xh,Ca,YZev),(Xh,Ca, YZew),(Xh,Ca,YZex),(Xh,Ca,YZey),(Xh,Ca,YZez),(Xh,Ca,YZfa),(Xh,Ca,YZfb),(Xh, Ca,YZfc),(Xh,Ca,YZfd),(Xh,Ca,YZfe),(Xh,Ca,YZff),(Xh,Ca,YZfg),(Xh,Ca,YZfh),(Xh, Ca,YZfi),(Xh,Ca,YZfj),(Xh,Ca,YZfk),(Xh,Ca,YZfl),(Xh,Ca,YZfm),(Xh,Ca,YZfn),(Xh, Ca,YZfo),(Xh,Ca,YZfp),(Xh,Ca,YZfq),(Xh,Ca,YZfr),(Xh,Ca,YZfs),(Xh,Ca,YZft),(Xh, Ca,YZfu),(Xh,Ca,YZfv),(Xh,Ca,YZfw),(Xh,Ca,YZfx),(Xh,Ca,YZfy),(Xh,Ca,YZfz),(Xh, Ca,YZga),(Xh,Ca,YZgb),(Xh,Ca,YZgc),(Xh,Ca,YZgd),(Xh,Ca,YZge),(Xh,Ca,YZgf), (Xh,Ca,YZgg),(Xh,Ca,YZgh),(Xh,Ca,YZgi),(Xh,Ca,YZgj),(Xh,Ca,YZgk),(Xh,Ca,YZgl), (Xh,Ca,YZgm),(Xh,Ca,YZgn),(Xh,Ca,YZgo),(Xh,Ca,YZgp),(Xh,Ca,YZgq),(Xh, Ca,YZgr),(Xh,Ca,YZgs),(Xh,Ca,YZgt),(Xh,Cb,YZa),(Xh,Cb,YZb),(Xh,Cb,YZc),(Xh, Cb,YZd),(Xh,Cb,YZe),(Xh,Cb,YZf),(Xh,Cb,YZg),(Xh,Cb,YZh),(Xh,Cb,YZi),(Xh,Cb, YZj),(Xh,Cb,YZk),(Xh,Cb,YZl),(Xh,Cb,YZm),(Xh,Cb,YZn),(Xh,Cb,YZo),(Xh,Cb,YZp), (Xh,Cb,YZq),(Xh,Cb,YZr),(Xh,Cb,YZs),(Xh,Cb,YZt),(Xh,Cb,YZu),(Xh,Cb,YZv),(Xh, Cb,YZw),(Xh,Cb,YZx),(Xh,Cb,YZy),(Xh,Cb,YZz),(Xh,Cb,YZaa),(Xh,Cb,YZab),(Xh, Cb,YZac),(Xh,Cb,YZad),(Xh,Cb,YZae),(Xh,Cb,YZaf),(Xh,Cb,YZag),(Xh,Cb,YZah), (Xh,Cb,YZai),(Xh,Cb,YZaj),(Xh,Cb,YZak),(Xh,Cb,YZal),(Xh,Cb,YZam),(Xh,Cb, YZan),(Xh,Cb,YZao),(Xh,Cb,YZap),(Xh,Cb,YZaq),(Xh,Cb,YZar),(Xh,Cb,YZas),(Xh, Cb,YZat),(Xh,Cb,YZau),(Xh,Cb,YZav),(Xh,Cb,YZaw),(Xh,Cb,YZax),(Xh,Cb,YZay), (Xh,Cb,YZaz),(Xh,Cb,YZba),(Xh,Cb,YZbb),(Xh,Cb,YZbc),(Xh,Cb,YZbd),(Xh,Cb,YZbe), (Xh,Cb,YZbf),(Xh,Cb,YZbg),(Xh,Cb,YZbh),(Xh,Cb,YZbi),(Xh,Cb,YZbj),(Xh,Cb, YZbk),(Xh,Cb,YZbl),(Xh,Cb,YZbm),(Xh,Cb,YZbn),(Xh,Cb,YZbo),(Xh,Cb,YZbp),(Xh, Cb,YZbq),(Xh,Cb,YZbr),(Xh,Cb,YZbs),(Xh,Cb,YZbt),(Xh,Cb,YZbu),(Xh,Cb,YZbv), (Xh,Cb,YZbw),(Xh,Cb,YZbx),(Xh,Cb,YZby),(Xh,Cb,YZbz),(Xh,Cb,YZca),(Xh,Cb, YZcb),(Xh,Cb,YZcc),(Xh,Cb,YZcd),(Xh,Cb,YZce),(Xh,Cb,YZcf),(Xh,Cb,YZcg),(Xh, Cb,YZch),(Xh,Cb,YZci),(Xh,Cb,YZcj),(Xh,Cb,YZck),(XSh,Cb,YZcl),(Xh,Cb,YZcm),(Xh, Cb,YZcn),(Xh,Cb,YZco),(Xh,Cb,YZcp),(Xh,Cb,YZcq),(Xh,Cb,YZcr),(Xh,Cb,YZcs), (Xh,Cb,YZct),(Xh,Cb,YZcu),(Xh,Cb,YZcv),(Xh,Cb,YZcw),(Xh,Cb,YZcx),(Xh,Cb, YZcy),(Xh,Cb,YZcz),(Xh,Cb,YZda),(Xh,Cb,YZdb),(Xh,Cb,YZdc),(Xh,Cb,YZdd),(Xh, Cb,YZde),(Xh,Cb,YZdf),(Xh,Cb,YZdg),(Xh,Cb,YZdh),(Xh,Cb,YZdi),(Xh,Cb,YZdj),(Xh, Cb,YZdk),(Xh,Cb,YZdl),(Xh,Cb,YZdm),(Xh,Cb,YZdn),(Xh,Cb,YZdo),(Xh,Cb,YZdp), (Xh,Cb,YZdq),(Xh,Cb,YZdr),(Xh,Cb,YZds),(Xh,Cb,Yzdt),(Xh,Cb,YZdu),(Xh,Cb, YZdv),(Xh,Cb,YZdw),(Xh,Cb,YZdx),(Xh,Cb,YZdy),(Xh,Cb,YZdz),(Xh,Cb,YZea),(Xh, Cb,YZeb),(Xh,Cb,YZec),(Xh,Cb,YZed),(Xh,Cb,YZee),(Xh,Cb,YZef),(Xh,Cb,YZeg), (Xh,Cb,YZeh),(Xh,Cb,YZei),(Xh,Cb,YZej),(Xh,Cb,YZek),(Xh,Cb,YZel),(Xh,Cb,YZem), (Xh,Cb,YZen),(Xh,Cb,YZeo),(Xh,Cb,YZep),(Xh,Cb,YZeq),(Xh,Cb,YZer),(Xh,Cb, YZes),(Xh,Cb,YZet),(Xh,Cb,YZeu),(Xh,Cb,YZev),(Xh,Cb,YZew),(Xh,Cb,YZex),(Xh, Cb,YZey),(Xh,Cb,YZez),(Xh,Cb,YZfa),(Xh,Cb,YZfb),(Xh,Cb,YZfc),(Xh,Cb,YZfd),(Xh, Cb,YZfe),(Xh,Cb,YZff),(Xh,Cb,YZfg),(Xh,Cb,YZfh),(Xh,Cb,YZfi),(Xh,Cb,YZfaj), (Xh,Cb,YZfk),(Xh,Cb,YZfl),(Xh,Cb,YZfm),(Xh,Cb,YZfn),(Xh,Cb,YZfo),(Xh,Cb,YZfp), (Xh,Cb,YZfq),(Xh,Cb,YZfr),(Xh,Cb,YZfs),(Xh,Cb,YZft),(Xh,Cb,YZfu),(Xh,Cb,YZfv), (Xh,Cb,YZfw),(Xh,Cb,YZfx),(Xh,Cb,YZfy),(Xh,Cb,YZfz),(Xh,Cb,YZga),(Xh,Cb, YZgb),(Xh,Cb,YZgc),(Xh,Cb,YZgd),(Xh,Cb,YZge),(Xh,Cb,YZgf),(Xh,Cb,YZgg),(Xh, Cb,YZgh),(Xh,Cb,YZgi),(Xh,Cb,YZgj),(Xh,Cb,YZgk),(Xh,Cb,YZgl),(Xh,Cb,YZgm), (Xh,Cb,YZgn),(Xh,Cb,YZgo),(Xh,Cb,YZgp),(Xh,Cb,YZgq),(Xh,Cb,YZgr),(Xh,Cb,YZgs), (Xh,Cb,YZgt),(Xh,Cc,YZa),(Xh,CNc,YZb),(Xh,Cc,YZc),(Xh,Cc,YZd),(Xh,Cc,YZe), (Xh,Cc,YZf),(Xh,Cc,YZg),(Xh,Cc,YZh),(Xh,Cc,YZi),(Xh,Cc,YZj),(Xh,Cc,YZk),(Xh, Cc,YZl),(Xh,Cc,YZm),(Xh,Cc,YZn),(Xh,Cc,YZo),(Xh,Cc,YZp),(Xh,Cc,YZq),(Xh,Cc, YZr),(Xh,Cc,YZs),(Xh,Cc,YZt),(Xh,Cc,YZu),(Xh,Cc,YZv),(Xh,Cc,YZw),(Xh,Cc,YZx), (Xh,Cc,YZy),(Xh,Cc,YZz),(Xh,Cc,YZaa),(Xh,Cc,YZab),(Xh,Cc,YZac),(Xh,Cc,YZad), (Xh,Cc,YZae),(Xh,Cc,YZaf),(Xh,Cc,YZag),(Xh,Cc,YZah),(Xh,Cc,YZai),(Xh,Cc, YZaj),(Xh,Cc,YZak),(Xh,Cc,YZal),(Xh,Cc,YZam),(Xh,Cc,YZan),(Xh,Cc,YZao),(Xh, Cc,YZap),(Xh,Cc,YZaq),(Xh,Cc,YZar),(Xh,Cc,YZas),(Xh,Cc,YZat),(Xh,Cc,YZau),(Xh, Cc,YZav),(Xh,Cc,YZaw),(Xh,Cc,YZax),(Xh,Cc,YZay),(Xh,Cc,YZaz),(Xh,Cc,YZba), (Xh,Cc,YZbb),(Xh,Cc,YZbc),(Xh,Cc,YZbd),(Xh,Cc,YZbe),(Xh,Cc,YZbf),(Xh,Cc,YZbg), (Xh,Cc,YZbh),(Xh,Cc,YZbi),(Xh,Cc,YZbj),(Xh,Cc,YZbk),(Xh,Cc,YZbl),(Xh,Cc, YZbm),(Xh,Cc,YZbn),(Xh,Cc,YZbo),(Xh,Cc,YZbp),(Xh,Cc,YZbq),(Xh,Cc,YZbr),(Xh, Cc,YZbs),(Xh,Cc,YZbt),(YXh,Cc,YZbu),(Xh,Cc,YZbv),(YXh,Cc,YZbw),(Xh,Cc,YZbx), (Xh,Cc,YZby),(Xh,Cc,YZbz),(Xh,Cc,YZea),(Xh,Cc,YZcb),(Xh,Cc,YZcc),(Xh,Cc,YZcd), (Xh,Cc,YZce),(Xh,Cc,YZcf),(Xh,Cc,YZcg),(Xh,Cc,YZch),(Xh,Cc,YZci),(Xh,Cc, YZcj),(Xh,Cc,YZck),(Xh,Cc,YZcl),(Xh,Cc,YZcm),(Xh,Cc,YZcn),(Xh,Cc,YZco),(Xh, Cc,YZcp),(YXh,Cc,YZcq),(YXh,Cc,YZcr),(Xh,Cc,YZcs),(Xh,Cc,YZct),(Xh,Cc,YZcu),(Xh, Cc,YZcv),(Xh,Cc,YZcw),(Xhn,Cc,YZcx),(Xh,Cc,YZcy),(Xh,Cc,YZcz),(Xh,Cc,YZda), (Xh,Cc,YZdb),(Xb,Cc,YZdc),(Xh,Cc,YZdd),(Xh,Cc,YZde),(Xh,Cc,YZdf),(Xh,Cc,YZdg), (Xh,Cc,YZdh),(Xh,Cc,YZdi),(Xh,Cc,YZdj),(Xh,Cc,YZdk),(Xh,Cc,YZdl),(Xh,Cc, YZdm),(Xh,Cc,YZdn),(Xh,Cc,YZdo),(Xh,Cc,YZdp),(Xh,Cc,YZdq),(Xh,Cc,YZdr),(Xh, Cc,YZds),(Xh,Cc,Yzdt),(Xh,Cc,YZdu),(Xh,Cc,YZdv),(Xh,Cc,YZdw),(Xh,Cc,YZdx), (Xh,Cc,YZdy),(Xh,Cc,YZdz),(Xh,Cc,YZea),(Xh,Cc,YZeb),(Xh,Cc,YZec),(Xh,Cc,YZed), (Xb,Cc,YZee),(Xh,Cc,YZef),(Xh,Cc,YZeg),(Xh,Cc,YZeh),(Xh,Cc,YZei),(Xh,Cc, YZej),(Xh,Cc,YZek),(Xh,Cc,YZel),(Xh,Cc,YZem),(Xh,Cc,YZen),(Xh,Cc,YZeo),(Xh, Cc,YZep),(Xh,Cc,YZeq),(Xh,Cc,YZer),(Xh,Cc,YZes),(Xh,Cc,YZet),(Xh,Cc,YZeu),(Xh, Cc,YZev),(Xh,Cc,YZew),(Xh,Cc,YZex),(Xh,Cc,YZey),(Xh,Cc,YZez),(Xh,Cc,YZfa), (Xh,Cc,YZfb),(Xh,Cc,YZfc),(Xh,Cc,YZfd),(Xh,Cc,YZfe),(Xh,Cc,YZff),(Xh,Cc,YZfg), (Xh,Cc,YZfh),(Xh,Cc,YZfi),(Xh,Cc,YZfj),(Xh,Cc,YZfk),(Xh,Cc,YZfl),(Xh,Cc,YZfm), (Xh,Cc,YZfn),(Xh,Cc,YZfo),(Xh,Cc,YZfp),(Xh,Cc,YZfq),(Xh,Cc,YZfr),(Xh,Cc,YZfs), (Xh,Cc,YZft),(Xh,Cc,YZfu),(Xh,Cc,YZfv),(Xh,Cc,YZfwv),(Xh,Cc,YZfx),(Xh,Cc, YZfy),(Xh,Cc,YZfz),(Xh,Cc,YZga),(Xh,Cc,YZgb),(Xh,Cc,YZgc),(Xh,Cc,YZgd),(Xh, Cc,YZge),(Xh,Cc,YZgf),(Xh,Cc,YZgg),(Xh,Cc,YZgh),(Xh,Cc,YZgi),(Xh,Cc,YZgj),(Xh, Cc,YZgk),(Xh,Cc,YZgl),(Xh,Cc,YZgm),(YXh,Cc,YZgn),(Xh,Cc,YZgo),(Xh,Cc,YZgp), (Xh,Cc,YZgq),(Xh,Cc,YZgr),(Xh,Cc,YZgs),(Xh,Cc,YZgt),(Xh,Cd,YZa),(Xh,Cd,YZb), (Xh,Cd,YZc),(Xh,Cd,YZd),(Xh,Cd,YZe),(Xh,Cd,YZf),(Xh,Cd,YZg),(Xh,Cd,YZh), (Xh,Cd,YZi),(Xh,Cd,YZj),(Xh,Cd,YZk),(Xh,Cd,YZl),(Xh,Cd,YZm),(Xh,Cd,YZn),(Xh, Cd,YZo),(Xh,Cd,YZp),(Xh,Cd,YZq),(Xh,Cd,YZr),(Xh,Cd,YZs),(Xh,Cd,YZt),(Xh,Cd, YZu),(Xh,Cd,YZv),(Xh,Cd,YZw),(Xh,Cd,YZx),(Xh,Cd,YZy),(Xh,Cd,YZz),(Xh,Cd, YZaa),(Xh,Cd,YZab),(Xh,Cd,YZac),(Xh,Cd,YZad),(Xh,Cd,YZae),(Xh,Cd,YZaf),(Xh, Cd,YZag),(Xh,Cd,YZah),(Xh,Cd,YZai),(Xh,Cd,YZaj),(Xh,Cd,YZak),(Xh,Cd,YZal),(Xh, Cd,YZam),(Xh,Cd,YZan),(Xh,Cd,YZao),(Xh,Cd,YZap),(Xh,Cd,YZaq),(Xh,Cd,YZar), (Xh,Cd,YZas),(Xh,Cd,YZat),(Xh,Cd,YZau),(Xh,Cd,YZav),(Xh,Cd,YZaw),(Xh,Cd, YZax),(Xh,Cd,YZay),(Xh,Cd,YZaz),(Xh,Cd,YZba),(Xh,Cd,YZbb),(Xh,Cd,YZbc),(Xh, Cd,YZbd),(Xh,Cd,YZbe),(Xb,Cd,YZbf),(Xh,Cd,YZbg),(Xh,Cd,YZbh),(Xh,Cd,YZbi), (Xh,Cd,YZbj),(Xh,Cd,YZbk),(Xh,Cd,YZbl),(Xh,Cd,YZbm),(Xh,Cd,YZbn),(Xh,Cd,YZbo), (Xh,Cd,YZbp),(Xh,Cd,YZbq),(Xh,Cd,YZbr),(Xh,Cd,YZbs),(YXh,Cd,YZbt),(Nh,Cd, YZbu),(Xh,Cd,YZbv),(Xh,Cd,YZbw),(Xh,Cd,YZbx),(Xh,Cd,YZby),(Xh,Cd,YZbz),(Xh, Cd,YZca),(Xh,Cd,YZcb),(Xh,Cd,YZcc),(Xh,Cd,YZcd),(Xh,Cd,YZce),(Xh,Cd,YZef), (Xh,Cd,YZcg),(Xh,Cd,YZch),(Xh,Cd,YZci),(Xh,Cd,YZcj),(Xh,Cd,YZck),(Xh,Cd,YZcl), (Xh,Cd,YZcm),(Xh,Cd,YZcn),(Xh,Cd,YZco),(Xh,Cd,YZcp),(Xh,Cd,YZcq),(Xh, Cd,YZcr),(Xh,Cd,YZcs),(Xh,Cd,YZct),(Xh,Cd,YZcu),(Xh,Cd,YZcv),(Xh,Cd,YZcw),(Xh, Cd,YZcx),(Xh,Cd,YZcy),(Xh,Cd,YZcz),(Xh,Cd,YZda),(Xh,Cd,YZdb),(Xh,Cd,YZdc), (YXh,Cd,YZdd),(Xh,Cd,YZde),(Xh,Cd,YZdf),(Xh,Cd,YZdg),(Xh,Cd,YZdh),(Xh,Cd, YZdi),(Xh,Cd,YZdj),(Xh,Cd,YZdk),(Xh,Cd,YZdl),(Xh,Cd,YZdm),(Xh,Cd,YZdn),(Xh, Cd,YZdo),(Xh,Cd,YZdp),(Xh,Cd,YZdq),(Xh,Cd,YZdr),(Xh,Cd,YZds),(Xh,Cd,Yzdt), (Xh,Cd,YZdu),(Xh,Cd,YZdv),(Xh,Cd,YZdw),(Xh,Cd,YZdx),(Xh,Cd,YZdy),(Xh,Cd, YZdz),(Xh,Cd,YZea),(Xh,Cd,YZeb),(Xh,Cd,YZec),(Xh,Cd,YZed),(Xh,Cd,YZee),(Xh, Cd,YZef),(Xh,Cd,YZeg),(Xh,Cd,YZeh),(Xh,Cd,YZei),(Xh,Cd,YZej),(Xh,Cd,YZek),(Xh, Cd,YZel),(Xh,Cd,YZem),(Xh,Cd,YZen),(Xh,Cd,YZeo),(Xh,Cd,YZep),(Xh,Cd,YZeq), (Xh,Cd,YZer),(Xh,Cd,YZes),(Xh,Cd,YZet),(Xh,Cd,YZeu),(Xh,Cd,YZev),(Xh,Cd, YZew),(Xh,Cd,YZex),(Xh,Cd,YZey),(Xh,Cd,YZcz),(Xh,Cd,YZfa),(Xh,Cd,YZfb),(Xh, Cd,YZfc),(Xh,Cd,YZfd),(Xh,Cd,YZfe),(Xh,Cd,YZff),(Xh,Cd,YZfg),(Xh,Cd,YZfh),(Xh, Cd,YZfi),(Xh,Cd,YZfj),(Xh,Cd,YZfk),(Xh,Cd,YZfl),(Xh,Cd,YZfm),(Xh,Cd,YZfn),(Xh, Cd,YZfo),(Xh,Cd,YZfp),(Xh,Cd,YZfq),(Xh,Cd,YZfr),(Xh,Cd,YZfs),(Xh,Cd,YZft), (Xh,Cd,YZfu),(Xh,Cd,YZfv),(Xh,Cd,YZfwv),(Xh,Cd,YZfx),(Xh,Cd,YZfy),(Xh,Cd,YZfz), (Xh,Cd,YZga),(Xh,Cd,YZgb),(Xh,Cd,YZgc),(Xh,Cd,YZgd),(Xh,Cd,YZge),(Xh,Cd, YZgf),(Xh,Cd,YZgg),(Xh,Cd,YZgh),(Xh,Cd,YZgi),(Xh,Cd,YZgj),(Xh,Cd,YZgk),(Xh, Cd,YZgl),(Xh,Cd,YZgm),(Xh,Cd,YZgn),(Xh,Cd,YZgo),(Xh,Cd,YZgp),(Xh,Cd,YZgq), (Xh,Cd,YZgr),(Xh,Cd,YZgs),(Xh,Cd,YZgt),(Xh,Ce,YZa),(Xh,Ce,YZb),(Xh,Ce,YZc), (Xh,Ce,YZd),(Xh,Ce,YZe),(Xh,Ce,YZf),(Xh,Ce,YZg),(Xh,Ce,YZh),(Xh,Ce,YZi),(Xh, Ce,YZj),(Xh,Ce,YZk),(Xh,Ce,Yl),(Xh,Ce,YZm),(Xh,Ce,YZn),(Xh,Ce,YZo),(Xh,Ce, YZp),(Xh,Ce,YZq),(Xh,Ce,YZr),(Xb,Ce,Ys),(Xh,Ce,YZt),(Xb,Ce,YZu),(Xh,Ce,YZv), (Xh,Ce,YZw),(Xh,Ce,YZx),(Xh,Ce,YZy),(Xh,Ce,YZz),(Xh,Ce,YZaa),(Xh,Ce,YZab), (Xh,Ce,YZac),(Xh,Ce,YZad),(Xh,Ce,YZae),(Xh,Ce,YZaf),(Xh,Ce,YZag),(Xh,Ce,YZah), (Xh,Ce,YZai),(Xh,Ce,YZaj),(Xh,Ce,YZak),(Xh,Ce,YZal),(Xh,Ce,YZam),(Xh,Ce, YZan),(Xh,Ce,YZao),(Xh,Ce,YZap),(Xh,Ce,YZaq),(Xh,Ce,YZar),(Xh,Ce,YZas),(Xh, Ce,YZat),(Xh,Ce,YZau),(Xh,Ce,YZav),(Xh,Ce,YZaw),(Xh,Ce,YZax),(Xh,Ce,YZay),(Xh, Ce,YZaz),(YXh,Ce,YZba),(Xh,Ce,YZbb),(Xh,Ce,YZbc),(Xh,Ce,YZbd),(Xh,Ce,YZbe), (Xh,Ce,YZbf),(Xh,Ce,YZbg),(Xh,Ce,YZbh),(Xh,Ce,YZbi),(Xh,Ce,YZbj),(Xh,Ce,YZbk), (Xh,Ce,YZbl),(YXh,Ce,YZbm),(Xh,Ce,YZbn),(Xh,Ce,YZbo),(Xh,Ce,YZbp),(Xh, Ce,YZbq),(Xh,Ce,YZbr),(Xh,Ce,YZbs),(Xh,Ce,YZbt),(Yh,Ce,YZbu),(Xh,Ce,YZbv),(Xh, Ce,YZbw),(Xh,Ce,YZbx),(Xh,Ce,YZby),(Xh,Ce,YZbz),(Xh,Ce,YZca),(Xh,Ce,YZcb), (Xh,Ce,YZcc),(Xh,Ce,YZcd),(Xh,Ce,YZce),(Xh,Ce,YZcf),(Xh,Ce,YZcg),(Xh,Ce, YZch),(Xh,Ce,YZci),(Xh,Ce,YZcj),(Xh,Ce,YZek),(Xh,Ce,YZcl),(Xh,Ce,YZem),(Xh, Ce,YZcn),(Xh,Ce,YZco),(Xh,Ce,YZcp),(Xh,Ce,YZcq),(Xh,Ce,YZcr),(Xh,Ce,YZcs),(Xh, Ce,YZct),(Xh,Ce,YZcu),(Xh,Ce,YZcv),(Xh,Ce,YZcw),(Xh,Ce,YZcx),(Xh,Ce,YZcy), (Xh,Ce,YZcz),(Xh,Ce,YZda),(Xh,Ce,YZdb),(Xh,Ce,YZdc),(Xh,Ce,YZdd),(Xh,Ce, YZde),(Xh,Ce,YZdf),(Xh,Ce,YZdg),(Xh,Ce,YZdh),(Xh,Ce,YZdi),(Xh,Ce,YZdj),(Xh, Ce,YZdk),(Xb,Ce,YZdl),(Xh,Ce,YZdm),(Xh,Ce,YZdn),(Xh,Ce,YZdo),(Xh,Ce,YZdp), (Xh,Ce,YZdq),(Xh,Ce,YZdr),(Xh,Ce,YZds),(Xh,Ce,Yzdt),(Xh,Ce,YZdu),(Xh,Ce,YZdv), (Xh,Ce,YZdw),(Xh,Ce,YZdx),(Xh,Ce,YZdy),(Xh,Ce,YZdz),(Xh,Ce,YZea),(Xh,Ce, YZeb),(Xh,Ce,YZec),(Xh,Ce,YZed),(Xh,Ce,YZee),(Xh,Ce,YZef),(Xh,Ce,YZeg),(Xh, Ce,YZeh),(Xh,Ce,YZei),(Xh,Ce,YZej),(Xh,Ce,YZek),(Xh,Ce,YZel),(Xh,Ce,YZem),(Xh, Ce,YZen),(Xh,Ce,YZeo),(Xh,Ce,YZep),(Xh,Ce,YZeq),(Xh,Ce,YZer),(Xh,Ce,YZes), (Xh,Ce,YZet),(Xh,Ce,YZeu),(Xh,Ce,YZev),(Xh,Ce,YZew),(Xh,Ce,YZex),(Xh,Ce,YZey), (Xh,Ce,YZez),(Xh,Ce,YZfa),(Xh,Ce,YZfb),(Xh,Ce,YZfc),(Xh,Ce,YZfd),(Xh,Ce, YZfe),(Xh,Ce,YZff),(Xh,Ce,YZfg),(Xh,Ce,YZfh),(Xh,Cc,YZfi),(Xh,Ce,YZfj),(Xh,Ce, YZfk),(Xh,Ce,YZfl),(Xh,Ce,YZfm),(Xh,Ce,YZfn),(Xh,Ce,YZfo),(Xh,Ce,YZfp),(Xh,Ce, YZfq),(Xh,Ce,YZfr),(Xh,Ce,YZfs),(Xh,Ce,YZft),(Xh,Ce,YZfu),(Xh,Ce,YZfv),(Xh,Ce, YZfw),(Xh,Ce,YZfx),(Xh,Ce,YZfy),(Xh,Ce,YZfz),(Xh,Ce,YZga),(Xh,Ce,YZgb),(Xh, Ce,YZgc),(Xh,Ce,YZgd),(Xh,Ce,YZge),(Xh,Ce,YZgf),(Xh,Ce,YZgg),(Xh,Ce,YZgh), (Xh,Ce,YZgi),(Xh,Ce,YZgj),(Xh,Ce,YZgk),(Xh,Ce,YZgl),(Xh,Ce,YZgm),(Xh,Ce,YZgn), (Xb,Ce,YZgo),(Xh,Ce,YZgp),(Xh,Ce,YZgq),(Xh,Ce,YZgr),(Xh,Ce,YZgs),(Xh,Ce, YZgt),(Xh,Cf,YZa),(Xh,Cf,YZb),(Xh,Cf,YZc),(Xh,Cf,YZd),(Xh,Cf,YZe),(Xh,Cf,YZf), (Xh,Cf,YZg),(Xh,Cf,YZh),(Xh,Cf,YZi),(Xh,Cf,YZj),(Xh,Cf,YZk),(Xh,Cf,YZl),(Xh,Cf, YZm),(Xh,Cf,YZn),(Xh,Cf,YZo),(YXh,Cf,YZp),(Yh,bCf,YZq),(Xh,Cf,YZr),(Xh,Cf,YZs), (Xh,Cf,YZt),(Xh,Cf,YZu),(YXh,Cf,YZv),(Xh,Cf,YZw),(Xh,Cf,YZx),(Xh,Cf,YZy),(Xh, Cf,YZz),(Xh,Cf,YZaa),(Xh,Cf,YZab),(Yh,Cf,YZac),(Xh,Cf,YZad),(Xh,Cf,YZae),(Xh, Cf,YZaf),(Xh,Cf,YZag),(Xh,Cf,YZah),(Xh,Cf,YZai),(Xh,Cf,YZaj),(Xh,Cf,YZak),(Xh, Cf,YZal),(Xh,Cf,YZam),(Xh,Cf,YZan),(Xh,Cf,YZao),(YXh,Cf,YZap),(Xh,Cf,YZaq),(Xh, Cf,YZar),(Xh,Cf,YZas),(Xh,Cf,YZat),(Xh,Cf,YZau),(Xh,Cf,YZav),(Xh,Cf,YZaw), (Xh,Cf,YZax),(Xh,Cf,YZay),(Xh,Cf,YZaz),(Xh,Cf,YZba),(Xh,Cf,YZbb),(Xh,Cf,YZbc), (Xh,Cf,YZbd),(Xh,Cf,YZbe),(Xh,Cf,YZbf),(Xh,Cf,YZbg),(Xh,Cf,YZbh),(Xh,Cf,YZbi), (YXh,Cf,YZbj),(Xh,Cf,YZbk),(YXh,Cf,YZbl),(YXh,Cf,YZbm),(Xh,Cf,YZbn),(Xh,Cf,YZbo), (Xh,Cf,YZbp),(Xh,Cf,YZbq),(Xh,Cf,YZbr),(Xh,Cf,YZbs),(Xh,Cf,YZbt),(Xh,Cf, YZbu),(Xh,Cf,YZbv),(Xh,Cf,YZbw),(Xh,Cf,YZbx),(Xh,Cf,YZby),(Xh,Cf,YZbz),(Xh, Cf,YZca),(Xh,Cf,YZcb),(Xh,Cf,YZcc),(Xh,Cf,YZcd),(Xh,Cf,YZce),(Xh,Cf,YZcf),(Xh, Cf,YZcg),(Xh,Cf,YZch),(Xh,Cf,YZci),(Xh,Cf,YZcj),(Xh,Cf,YZck),(Xh,Cf,YZcl),(Xh, Cf,YZcm),(Xh,Cf,YZcn),(Xh,Cf,YZco),(Xh,Cf,YZcp),(Xh,Cf,YZcq),(Xh,Cf,YZcr),(Xh, Cf,YZcs),(Xh,Cf,YZct),(Xh,Cf,YZcu),(Xh,Cf,YZcv),(Xh,Cf,YZcw),(Xh,Cf,YZcx), (Xh,Cf,YZcy),(YXh,Cf,YZcz),(Xh,Cf,YZda),(Xh,Cf,YZdb),(Xh,Cf,YZdc),(Xh,Cf,YZd), (Xh,Cf,YZde),(Xh,Cf,YZdf),(Xh,Cf,YZdg),(Xh,Cf,YZdh),(Xh,Cf,YZdi),(Xh,Cf,YZdj), (Xh,Cf,YZdk),(Xh,Cf,YZdl),(Xh,Cf,YZdm),(Xh,Cf,YZdn),(Xh,Cf,YZdo),(Xh,Cf, YZdp),(Xh,Cf,YZdq),(Xh,Cf,YZdr),(Xh,Cf,YZds),(Xh,Cf,Yzdt),(Xh,Cf,YZdu),(Xh,Cf, YZdv),(Xh,Cf,YZdw),(Xh,Cf,YZdx),(Xh,Cf,YZdy),(Xh,Cf,YZdz),(Xh,Cf,YZea),(Xb, Cf,YZeb),(Xh,Cf,YZec),(Xh,Cf,YZed),(Xh,Cf,YZee),(Xh,Cf,YZef),(Xh,Cf,YZeg),(Xh, Cf,YZeh),(Xh,Cf,YZei),(Xh,Cf,YZej),(Xh,Cf,YZek),(Xh,Cf,YZel),(Xh,Cf,YZem),(Xh, Cf,YZen),(Xh,Cf,YZeo),(Xh,Cf,YZep),(Xh,Cf,YZeq),(Xh,Cf,YZer),(Xh,Cf,YZes),(Xh, Cf,YZet),(Xh,Cf,YZeu),(Xh,Cf,YZev),(Xh,Cf,YZew),(Xh,Cf,YZex),(Xh,Cf,YZey), (Xh,Cf,YZez),(Xh,Cf,YZfa),(Xh,Cf,YZfb),(Xh,Cf,YZfc),(Xh,Cf,YZfd),(Xh,Cf,YZfe), (Xh,Cf,YZff),(Xh,Cf,YZfg),(Xh,Cf,YZfh),(Xh,Cf,YZfi),(Xh,Cf,YZfj,(Xh,Cf,YZfk),(Xh, Cf,YZfl),(Xh,Cf,YZfm),(Xh,Cf,YZfn),(Xh,Cf,YZfo),(Xh,Cf,YZfp),(Xh,Cf,YZfq),(Xh, Cf,YZfr),(Xb,Cf,YZfs),(Xh,Cf,YZft),(Xh,Cf,YZfu),(Xh,Cf,YZfv),(Xh,Cf,YZfw),(Xh, Cf,YZfx),(Xh,Cf,YZfy),(Xh,Cf,YZfz),(Xh,Cf,YZga),(Xh,Cf,YZgb),(Xh,Cf,YZgc), (Xh,Cf,YZgd),(YXh,Cf,YZge),(Xh,Cf,YZgf),(Xh,Cf,YZgg),(Xh,Cf,YZgh),(Xh,Cf,YZgi), (Xh,Cf,YZgj),(Xh,Cf,YZgk),(Xh,Cf,YZgl),(Xh,Cf,YZgm),(Xh,Cf,YZgn),(Xh,Cf,YZgo), (Xh,Cf,YZgp),(Xh,Cf,YZgq),(Xh,Cf,YZgr),(Xh,Cf,YZgs),(Xh,Cf,YZgt), (Xi,Ca,YZa),(Xi,Ca,YZb),(Xi,Ca,YZc),(Xi,Ca,YZd),(Xi,Ca,YZe),(Xi,Ca,YZf),(Xi,Ca, YZg),(Xi,Ca,YZh),(Xi,Ca,YZi),(Xi,Ca,YZj),(Xi,Ca,YZk),(Xi,Ca,YZl),(Xi,Ca,YZm),(Xi, Ca,YZn),(Xi,Ca,YZo),(Xi,Ca,YZp),(Xi,Ca,YZq),(Xi,Ca,YZr),(Xi,Ca,YZs),(Xi,Ca, YZt),(Xi,Ca,YZu),(Xi,Ca,YZv),(Xi,Ca,YZw),(Xi,Ca,YZx),(Xi,Ca,YZy),(Xi,Ca,YZz),(Xi, Ca,YZaa),(Xi,Ca,YZab),(Xi,Ca,YZac),(Xi,Ca,YZad),(Xi,Ca,YZae),(Xi,Ca,YZaf),(Xi, Ca,YZag),(Xi,Ca,YZah),(Xi,Ca,YZai),(Xi,Ca,YZaj),(Xi,Ca,YZak),(Xi,Ca,YZal),(Xi, Ca,YZam),(Xi,Ca,YZan),(Xi,Ca,YZao),(Xi,Ca,YZap),(Xi,Ca,YZaq),(Xi,Ca,YZar),(Xi, Ca,YZas),(Xi,Ca,YZat),(Xi,Ca,YZau),(Xi,Ca,YZav),(Xi,Ca,YZaw),(Xi,Ca,YZax),(Xi, Ca,YZay),(Xi,Ca,YZaz),(Xi,Ca,YZba),(Xi,Ca,YZbb),(Xi,Ca,YZbc),(Xi,Ca,YZbd),(Xi, Ca,YZbe),(Xi,Ca,YZbf),(Xi,Ca,YZbg),(Xi,Ca,YZbh),(Xi,Ca,YZbi),(Xi,Ca,YZbj),(Xi, Ca,YZbk),(Xi,Ca,YZbl),(Xi,Ca,YZbm),(Xi,Ca,YZbn),(Xi,Ca,YZbo),(Xi,Ca,YZbp),(Xi, Ca,YZbq),(Xi,Ca,YZbr),(Xi,Ca,YZbs),(Xi,Ca,YZbt),(Xi,Ca,YZbu),(Xi,Ca,YZbv),(Xi, Ca,YZbw),(Xi,Ca,YZbx),(Xi,Ca,YZby),(Xi,Ca,YZbz),(Xi,Ca,YZca),(Xi,Ca,YZcb),(Xi, Ca,YZcc),(Xi,Ca,YZcd),(Xi,Ca,YZce),(Xi,Ca,YZcf),(Xi,Ca,YZcg),(Xi,Ca,YZch),(Xi, Ca,YZci),(Xi,Ca,YZcj),(Xi,Ca,YZck),(Xi,Ca,YZcl),(Xi,Ca,YZcm),(Xi,Ca,YZcn),(Xi,Ca, YZcu),(Xi,Ca,YZcv),(Xi,Ca,YZcw),(Xi,Ca,YZcx),(Xi,Ca,YZcy),(Xi,Ca,YZcz),(Xi,Ca, YZda),(Xi,Ca,YZdb),(Xi,Ca,YZdc),(Xi,Ca,YZdd),(Xi,Ca,YZde),(Xi,Ca,YZdf),(Xi,Ca, YZdg),(Xi,Ca,YZdh),(Xi,Ca,YZdi),(Xi,Ca,YZdj),(Xi,Ca,YZdk),(Xi,Ca,YZdl),(Xi,Ca, YZdm),(Xi,Ca,YZdn),(Xi,Ca,YZdo),(Xi,Ca,YZdp),(Xi,Ca,YZdq),(Xi,Ca,YZdr),(Xi,Ca, YZds),(Xi,Ca,Yzdt),(Xi,Ca,YZdu),(Xi,Ca,YZdv),(Xi,Ca,YZdw),(Xi,Ca,YZdx),(Xi,Ca, YZdy),(Xi,Ca,YZdz),(Xi,Ca,YZea),(Xi,Ca,YZeb),(Xi,Ca,YZec),(Xi,Ca,YZed),(Xi,Ca, YZee),(Xi,Ca,YZef),(Xi,Ca,YZeg),(Xi,Ca,YZeh),(Xi,Ca,YZei),(Xi,Ca,YZej),(Xi,Ca, YZek),(Xi,Ca,YZel),(Xi,Ca,YZem),(Xi,Ca,YZen),(Xi,Ca,YZeo),(Xi,Ca,YZep),(Xi,Ca, YZeq),(Xi,Ca,YZer),(Xi,Ca,YZes),(Xi,Ca,YZet),(Xi,Ca,YZeu),(Xi,Ca,YZev),(Xi,Ca, YZew),(Xi,Ca,YZex),(Xi,Ca,YZey),(Xi,Ca,YZez),(Xi,Ca,YZfa),(Xi,Ca,YZfb),(Xi,Ca, YZfc),(Xi,Ca,YZfd),(Xi,Ca,YZfe),(Xi,Ca,YZff),(Xi,Ca,YZfg),(Xi,Ca,YZfh),(Xi,Ca,YZfi), (Xi,Ca,YZfj),(Xi,Ca,YZfk),(Xi,Ca,YZfl),(Xi,Ca,YZfm),(Xi,Ca,YZfn),(Xi,Ca,YZfo), (Xi,Ca,YZfp),(Xi,Ca,YZfq),(Xi,Ca,YZfr),(Xi,Ca,YZfs),(Xi,Ca,YZft),(Xi,Ca,YZfu), (Xi,Ca,YZfv),(Xi,Ca,YZfwv),(Xi,Ca,YZfx),(Xi,Ca,YZfy),(Xi,Ca,YZfz),(Xi,Ca,YZga),(Xi, Ca,YZgb),(Xi,Ca,YZgc),(Xi,Ca,YZgd),(Xi,Ca,YZge),(Xi,Ca,YZgf),(Xi,Ca,YZgg),(Xi, Ca,YZgh),(Xi,Ca,YZgi),(Xi,Ca,YZgj),(Xi,Ca,YZgk),(Xi,Ca,YZgl),(Xi,Ca,YZgm),(Xi, Ca,YZgn),(Xi,Ca,YZgo),(Xi,Ca,YZgp),(Xi,Ca,YZgq),(Xi,Ca,YZgr),(Xi,Ca,YZgs),(Xi, Ca,YZgt),(Xi,Cb,YZa),(Xi,Cb,YZb),(Xi,Cb,YZc),(Xi,Cb,YZd),(Xi,Cb,YZc),(Xi,Cb, YZf),(Xi,Cb,YZg),(Xi,Cb,YZh),(Xi,Cb,YZi),(Xi,Cb,YZj),(Xi,Cb,YZk),(Xi,Cb,YZl),(Xi, Cb,YZm),(Xi,Cb,YZn),(Xi,Cb,YZo),(Xi,Cb,YZp),(Xi,Cb,YZq),(Xi,Cb,YZr),(Xi,Cb, YZs),(Xi,Cb,YZt),(Xi,Cb,YZu),(Xi,Cb,YZv),(Xi,Cb,YZw),(Xi,Cb,YZx),(Xi,Cb,YZy), (Xi,Cb,YZz),(Xi,Cb,YZaa),(Xi,Cb,YZab),(Xi,Cb,YZac),(Xi,Cb,YZad),(Xi,Cb,YZae), (Xi,Cb,YZaf),(Xi,Cb,YZag),(Xi,Cb,YZah),(Xi,Cb,YZai),(Xi,Cb,YZaj),(Xi,Cb,YZak), (Xi,Cb,YZal),(Xi,Cb,YZam),(Xi,Cb,YZan),(Xi,Cb,YZao),(Xi,Cb,YZap),(Xi,Cb,YZaq), (Xi,Cb,YZar),(Xi,Cb,YZas),(Xi,Cb,YZat),(Xi,Cb,YZau),(Xi,Cb,YZav),(Xi,Cb,YZaw), (Xi,Cb,YZax),(Xi,Cb,YZay),(Xi,Cb,YZaz),(Xi,Cb,YZba),(Xi,Cb,YZbb),(Xi,Cb,YZbc), (Xi,Cb,YZbd),(Xi,Cb,YZbe),(Xi,Cb,YZbf),(Xi,Cb,YZbg),(Xi,Cb,YZbh),(Xi,Cb,YZbi), (Xi,Cb,YZbj),(Xi,Cb,YZbk),(Xi,Cb,YZbl),(Xi,Cb,YZbm),(Xi,Cb,YZbn),(Xi,Cb,YZbo), (Xi,Cb,YZbp),(Xi,Cb,YZbq),(Xi,Cb,YZbr),(Xi,Cb,YZbs),(Xi,Cb,YZbt),(Xi,Cb,YZbu), (Xi,Cb,YZbv),(Xi,Cb,YZbw),(Xi,Cb,YZbx),(Xi,Cb,YZby),(Xi,Cb,YZbz),(Xi,Cb, YZca),(Xi,Cb,YZcb),(Xi,Cb,YZcc),(Xi,Cb,YZcd),(Xi,Cb,YZce),(Xi,Cb,YZcf),(Xi,Cb, YZcg),(Xi,Cb,YZch),(Xi,Cb,YZci),(Xi,Cb,YZcj),(Xi,Cb,YZck),(Xi,Cb,YZcl),(Xi,Cb, YZcm),(Xi,Cb,YZcn),(Xi,Cb,YZco),(Xi,Cb,YZcp),(Xi,Cb,YZcq),(Xi,Cb,YZcr),(Xi,Cb, YZcs),(Xi,Cb,YZct),(Xi,Cb,YZcu),(Xi,Cb,YZcv),(Xi,Cb,YZcw),(Xi,Cb,YZcx),(Xi, Cb,YZcy),(Xi,Cb,YZcz),(Xi,Cb,YZda),(Xi,Cb,YZdb),(Xi,Cb,YZdc),(Xi,Cb,YZdd),(Xi, Cb,YZde,),(Xi,Cb,YZdf),(Xi,Cb,YZdg),(Xi,Cb,YZdh),(Xi,Cb,YZdi),(Xi,Cb,YZdj),(Xi, Cb,YZdk),(Xi,Cb,YZdl),(Xi,Cb,YZdm),(Xi,Cb,YZdn),(Xi,Cb,YZdo),(Xi,Cb,MYZdp),(Xi, Cb,YZdq),(Xi,Cb,YZdr),(Xi,Cb,YZds),(Xi,Cb,Yzdt),(Xi,Cb,YZdu),(Xi,Cb,YZdv),(Xi, Cb,YZdw),(Xi,Cb,YZdx),(Xi,Cb,YZdy),(Xi,Cb,YZdz),(Xi,Cb,YZea),(Xi,Cb,YZeb), (Xi,Cb,YZec),(Xi,CbYZed),(Xi,Cb,YZee),(Xi,Cb,YZef),(Xi,Cb,YZeg),(Xi,Cb,YZeh), (Xi,Cb,YZei),(Xi,Cb,YZej),(Xi,Cb,YZek),(Xi,Cb,YZel),(Xi,Cb,YZem),(Xi,Cb,YZen), (Xi,Cb,YZeo),(Xi,Cb,YZep),(Xi,Cb,YZeq),(Xi,Cb,YZer),(Xi,Cb,YZes),(Xi,Cb,YZet), (Xi,Cb,YZeu),(Xi,Cb,YZev),(Xi,Cb,YZew),(Xi,Cb,YZex),(Xi,Cb,YZey),(Xi,Cb,YZez), (Xi,Cb,YZfa),(Xi,Cb,YZfb),(Xi,Cb,YZfc),(Xi,Cb,YZfd),(Xi,Cb,YZfe),(Xi,Cb,YZff), (Xi,Cb,YZfg),(Xi,Cb,YZfh),(Xi,Cb,YZfi),(Xi,Cb,YZfj),(Xi,Cb,YZfk),(Xi,Cb,YZfl),(Xi, Cb,YZfm),(Xi,Cb,YZfn),(Xi,Cb,YZfo),(Xi,Cb,YZfip),(Xi,Cb,YZfq),(Xi,Cb,YZfr),(Xi, Cb,YZfs),(Xi,Cb,YZft),(Xi,Cb,YZfu),(Xi,Cb,YZfv),(Xi,Cb,YZfw),(Xi,Cb,YZfx),(Xi, Cb,YZfy),(Xi,Cb,YZfz),(Xi,Cb,YZga),(Xi,Cb,YZgb),(Xi,Cb,YZgc),(Xi,Cb,YZgd),(Xi, Cb,YZge),(Xi,Cb,YZgf),(Xi,Cb,YZgg),(Xi,Cb,YZgh),(Xi,Cb,YZgi),(Xi,Cb,YZgj),(Xi, Cb,YZgk),(Xi,Cb,YZgl),(Xi,Cb,YZgm),(Xi,Cb,YZgn),(Xi,Cb,YZgo),(Xi,Cb,YZgp),(Xi, Cb,YZgq),(Xi,Cb,YZgr),(Xi,Cb,YZgs),(Xi,Cb,YZgt),(Xi,Cc,YZa),(Xi,Ce,YZb),(Xi, Cc,YZc),(Xi,Cc,YZd),(Xi,Cc,YZe),(Xi,Cc,YZf),(Xi,Cc,YZg),(Xi,Cc,YZh),(Xi,Cc,YZi), (Xi,Cc,YZj),(Xi,Cc,YZk),(Xi,Cc,YZl),(Xi,Cc,YZm),(Xi,Cc,YZn),(Xi,Cc,YZo),(Xi, Ce,YZp),(Xi,Cc,YZq),(Xi,Cc,YZr),(Xi,Cc,YZs),(Xi,Cc,YZt),(Xi,Cc,YZu),(Xi,Cc,YZv), (Xi,Cc,YZw),(Xi,Ce,YZx),(Xi,Cc,YZy),(Xi,Cc,YZz),(Xi,Cc,YZaa),(Xi,Cc,YZab),(Xi, Cc,YZac),(Xi,Cc,YZad),(Xi,Cc,YZae),(Xi,Cc,YZaf),(Xi,Cc,YZag),(Xi,Cc,YZah),(Xi, Cc,YZai),(Xi,Cc,YZaj),(Xi,Cc,YZak),(Xi,Cc,YZal),(Xi,Cc,YZam),(Xi,Cc,YZan),(Xi, Cc,YZao),(Xi,Cc,YZap),(Xi,Cc,YZaq),(Xi,Cc,YZar),(Xi,Cc,YZas),(Xi,Ce,YZat),(Xi,Cc, YZau),(Xi,Cc,YZav),(Xi,Cc,YZaw),(Xi,Cc,YZax),(Xi,Cc,YZay),(Xi,Cc,YZaz),(Xi,Cc, YZba),(Xi,Cc,YZbb),(Xi,Cc,YZbc),(Xi,Cc,YZbd),(Xi,Cc,YZbe),(Xi,Cc,YZbf),(Xi,Cc, YZbg),(Xi,Cc,YZbh),(Xi,Cc,YZbi),(Xi,Cc,YZbj),(Xi,Cc,YZbk),(Xi,Cc,YZbl),(Xi,Cc, YZbm),(Xi,Cc,YZbn),(Xi,Cc,YZbo),(Xi,Cc,YZbp),(Xi,Cc,YZbq),(Xi,Cc,YZbr),(Xi,Cc, YZbs),(Xi,Cc,YZbt),(Xi,Cc,YZbu),(Xi,Cc,YZbv),(Xi,Cc,YZbw),(Xi,Cc,YZbx),(Xi, Cc,YZby),(Xi,Cc,YZbz),(Xi,Cc,YZca),(Xi,Cc,YZcb),(Xi,Cc,YZcc),(Xi,Cc,YZcd),(Xi, Cc,YZce),(Xi,Cc,YZcf),(Xi,Cc,YZcg),(Xi,Cc,YZch),(Xi,Cc,YZci),(Xi,Cc,YZcj),(Xi,Cc, YZck),(Xi,Cc,YZcl),(Xi,Cc,YZcm),(Xi,Cc,YZcn),(Xi,Cc,YZco),(Xi,Cc,YZcp),(Xi,Cc, YZeq),(Xi,Cc,YZcr),(Xi,Cc,YZcs),(Xi,Ce,YZct),(Xi,Cc,YZcu),(Xi,Cc,YZcv),(Xi,Ce, YZcw),(Xi,Cc,YZcx),(Xi,Cc,YZcy),(Xi,Cc,YZcz),(Xi,Cc,YZda),(Xi,Cc,YZdb),(Xi,Cc, YZdc),(Xi,Cc,YZdd),(Xi,Cc,YZde),(Xi,Cc,YZdif),(Xi,Cc,YZdg),(Xi,Cc,YZdh),(Xi,Cc, YZdi),(Xi,Cc,YZdj),(Xi,Cc,YZdk),(Xi,Cc,YZdl),(Xi,Cc,YZdm),(Xi,Cc,YZdn),(Xi,Cc, YZdo),(Xi,Cc,YZdp),(Xi,Cc,YZdq),(Xi,Cc,YZdr),(Xi,Cc,YZds),(Xi,Cc,Yzdt),(Xi,Cc, YZdu),(Xi,Cc,YZdv),(Xi,Cc,YZdw),(Xi,Cc,YZdx),(Xi,Cc,YZdy),(Xi,Cc,YZdz),(Xi,Cc, YZea),(Xi,Cc,YZeb),(Xi,Cc,YZec),(Xi,Cc,YZed),(Xi,Cc,YZee),(Xi,Cc,YZef),(Xi,Ce YZeg),(Xi,Cc,YZeh),(Xi,Cc,YZei),(Xi,Cc,YZej),(Xi,Cc,YZek),(Xi,Cc,YZel),(Xi,Cc, YZem),(Xi,Cc,YZen),(Xi,Cc,YZeo),(Xi,Cc,YZep),(Xi,Cc,YZeq),(Xi,Cc,YZer),(Xi,Cc, YZes),(Xi,Cc,YZet),(Xi,Cc,YZeu),(Xi,Cc,YZev),(Xi,Cc,YZew),(Xi,Cc,YZex),(Xi,Cc, YZey),(Xi,Cc,YZez),(Xi,Cc,YZfa),(Xi,Cc,YZfb),(Xi,Cc,YZfc),(Xi,Cc,YZfd),(Xi,Cc,YZfe), (Xi,Cc,YZff),(Xi,c,YZfg),(Xi,Cc,YZfh),(Xi,Cc,YZfi),(Xi,Cc,YZfj),(Xi,Cc,YZfk), (Xi,Cc,YZfl),(Xi,Cc,YZfm),(Xi,Cc,YZfn),(Xi,Cc,YZfo),(Xi,Cc,YZfp),(Xi,Cc,YZfq), (Xi,Cc,YZfr),(Xi,Cc,YZfs),(Xi,Cc,YZft),(Xi,Cc,YZfu),(Xi,Cc,YZfv),(Xi,Cc,YZfw),(Xi, Cc,YZfx),(Xi,Cc,YZfy),(Xi,Cc,YZfz),(Xi,Cc,YZga),(Xi,Cc,YZgb),(Xi,Cc,YZgc),(Xi, Cc,YZgd),(Xi,Cc,YZge),(Xi,Cc,YZgf),(Xi,Cc,YZgg),(Xi,Cc,YZgh),(Xi,Cc,YZgi),(Xi, Cc,YZgj),(Xi,Cc,YZgk),(Xi,Cc,YZgl),(Xi,Cc,YZgm),(Xi,Cc,YZgn),(Xi,Cc,YZgo),(Xi, Cc,YZgp),(Xi,Cc,YZgq),(Xi,Cc,YZgr),(Xi,Cc,YZgs),(Xi,Cc,YZgt),(Xi,Cd,YZa),(Xi,Cd, YZb),(Xi,Cd,YZc),(Xi,Cd,YZd),(Xi,Cd,YZe),(Xi,Cd,YZf),(Xi,Cd,YZg),(Xi,Cd,YZh), (Xi,Cd,YZi),(Xi,Cd,YZj),(Xi,Cd,YZk),(Xi,Cd,YZl),(Xi,Cd,YZm),(Xi,Cd,YZn),(Xi,Cd, YZo),(Xi,Cd,YZp),(Xi,Cd,YZq),(Xi,Cd,YZr),(Xi,Cd,YZs),(Xi,Cd,YZt),(Xi,Cd,YZu), (Xi,Cd,YZv),(Xi,Cd,YZw),(Xi,Cd,YZx),(Xi,Cd,YZy),(Xi,Cd,YZz),(Xi,Cd,YZaa),(Xi, Cd,YZab),(Xi,Cd,YZac),(Xi,Cd,YZad),(Xi,Cd,YZae),(Xi,Cd,YZaf),(Xi,Cd,YZag),(Xi, Cd,YZah),(Xi,Cd,YZai),(Xi,Cd,YZaj),(Xi,Cd,YZak),(Xi,Cd,YZal),(Xi,Cd,YZam),(Xi, Cd,YZan),(Xi,Cd,YZao),(Xi,Cd,YZap),(Xi,Cd,YZaq),(Xi,Cd,YZar),(Xi,Cd,YZas),(Xi, Cd,YZat),(Xi,Cd,YZau),(Xi,Cd,YZav),(Xi,Cd,YZaw),(Xi,Cd,YZax),(Xi,Cd,YZay),(Xi, Cd,YZaz),(Xi,Cd,YZba),(Xi,Cd,YZbb),(Xi,Cd,YZbc),(Xi,Cd,YZbd),(Xi,Cd,YZbe),(Xi, Cd,YZbf),(Xi,Cd,YZbg),(Xi,Cd,YZbh),(Xi,Cd,YZbi),(Xi,Cd,YZbj),(Xi,Cd,YZbk),(Xi, Cd,YZbl),(Xi,Cd,YZbm),(Xi,Cd,YZbn),(Xi,Cd,YZbo),(Xi,Cd,YZbp),(Xi,Cd,YZbq),(Xi, Cd,YZbr),(Xi,Cd,YZbs),(Xi,Cd,YZbt),(Xi,Cd,YZbu),(Xi,Cd,YZbv),(Xi,Cd,YZbw), (Xi,Cd,YZbx),(Xi,Cd,YZby),(Xi,Cd,YZbz),(Xi,Cd,YZca),(Xi,Cd,YZcb),(Xi,Cd,YZcc), (Xi,Cd,YZcd),(Xi,Cd,YZce),(Xi,Cd,YZcf),(Xi,Cd,YZcg),(Xi,Cd,YZch),(Xi,Cd,YZci), (Xi,Cd,YZcj),(Xi,Cd,YZck),(Xi,Cd,YZcl),(Xi,Cd,YZcm),(Xi,Cd,YZcn),(Xi,Cd,YZco), (Xi,Cd,YZcp),(Xi,Cd,YZcq),(Xi,Cd,YZcr),(Xi,Cd,YZcs),(Xi,Cd,YZct),(Xi,Cd,YZcu),(Xi, Cd,YZcv),(Xi,Cd,YZcw),(Xi,Cd,YZcx),(Xi,Cd,YZcy),(Xi,Cd,YZcz),(Xi,Cd,YZda), (Xi,Cd,YZdb),(Xi,Cd,YZdc),(Xi,Cd,YZdd),(Xi,Cd,YZde),(Xi,Cd,YZdf),(Xi,Cd,YZdg), (Xi,Cd,YZdh),(Xi,Cd,YZdi),(Xi,Cd,YZdj),(Xi,Cd,YZdk),(Xi,Cd,YZdl),(Xi,Cd,YZdm), (Xi,Cd,YZdn),(Xi,Cd,YZdo),(Xi,Cd,YZdp),(Xi,Cd,YZdq),(Xi,Cd,YZdr),(Xi,Cd,YZds), (Xi,Cd,Yzdt),(Xi,Cd,YZdu),(Xi,Cd,YZdv),(Xi,Cd,YZdw),(Xi,Cd,YZdx),(Xi,Cd,YZdy), (Xi,Cd,YZdz),(Xi,Cd,YZea),(Xi,Cd,YZeb),(Xi,Cd,YZec),(Xi,Cd,YZed),(Xi,Cd,YZee), (Xi,Cd,YZef),(Xi,Cd,YZeg),(Xi,Cd,YZeh),(Xi,Cd,YZei),(Xi,Cd,YZej),(Xi,Cd,YZek), (Xi,Cd,YZel),(Xi,Cd,YZem),(Xi,Cd,YZen),(Xi,Cd,YZeo),(Xi,Cd,YZep),(Xi,Cd, YZeq),(Xi,Cd,YZer),(Xi,Cd,YZes),(Xi,Cd,YZet),(Xi,Cd,YZeu),(Xi,Cd,YZev),(Xi,Cd, YZew),(Xi,Cd,YZex),(Xi,Cd,YZey),(Xi,Cd,YZez),(Xi,Cd,YZfa),(Xi,Cd,YZfb),(Xi,Cd, YZfc),(Xi,Cd,YZfd),(Xi,Cd,YZfe),(Xi,Cd,YZff),(Xi,Cd,YZfg),(Xi,Cd,YZfh),(Xi,Cd, YZfi),(Xi,Cd,YZfj),(Xi,Cd,YZfk),(Xi,Cd,YZfl),(Xi,Cd,YZfm),(Xi,Cd,YZfn),(Xi,Cd,YZfo), (Xi,Cd,YZfp),(Xi,Cd,YZfq),(Yj,Cd,YZfr),(Xi,Cd,YZfs),(Xi,Cd,YZft),(Xi,Cd,YZfu), (Xi,Cd,YZfv),(Xi,Cd,YZfwv),(Xi,Cd,YZfx),(Xi,Cd,YZfy),(Xi,Cd,YZfz),(Xi,Cd,YZga), (Xi,Cd,YZgb),(Xi,Cd,YZgc),(Xi,Cd,YZgd),(Xi,Cd,YZge),(Xi,Cd,YZgf),(Xi,Cd,YZgg), (Xi,Cd,YZgh),(Xi,Cd,YZgi),(Xi,Cd,YZgj),(Xi,Cd,YZgk),(Xi,Cd,YZgl),(Xi,Cd,YZgm), (Xi,Cd,YZgn),(Xi,Cd,YZgo),(Xi,Cd,YZgp),(Xi,Cd,YZgq),(Xi,Cd,YZgr),(Xi,Cd,YZgs), (Xi,Cd,YZgt),(Xi,Ce,YZa),(Xi,Ce,YZb),(Xi,Ce,YZc),(Xi,Ce,YZd),(Xi,Ce,YZe),(Xi, Ce,YZf),(Xi,Ce,YZg),(Xi,Ce,YZh),(Xi,Ce,YZi),(Xi,Ce,YZj),(Xi,Ce,YZk),(Xi,Ce,YZl), (Xi,Ce,YZm),(Xi,Ce,YZn),(Xi,Ce,YZo),(Xi,Ce,YZp),(Xi,Ce,YZq),(Xi,Ce,YZr),(Xi, Ce,YZs),(Xi,Ce,YZt),(Xi,Ce,YZu),(Xi,Ce,YZv),(Xi,Ce,YZw),(Xi,Ce,YZx),(Xi,Ce,YZy), (Xi,Ce,YZz),(Xi,Ce,YZaa),(Xi,Ce,YZab),(Xi,Ce,YZac),(Xi,Ce,YZad),(Xi,Ce,YZae), (Xi,Ce,YZaf),(Xi,Ce,YZag),(Xi,Ce,YZah),(Xi,Ce,YZai),(Xi,Ce,YZaj),(Xi,Ce,YZak), (Xi,Ce,YZal),(Xi,Ce,YZam),(Xi,Ce,YZan),(Xi,Ce,YZao),(Xi,Ce,YZap),(Xi,Ce,YZaq), (Xi,Ce,YZar),(Xi,Ce,YZas),(Xi,Ce,YZat),(Xi,Ce,YZau),(Xi,Ce,YZav),(Xi,Ce,YZaw), (Xi,Ce,YZax),(Xi,Ce,YZay),(Xi,Ce,YZaz),(Xi,Ce,YZba),(Xi,Ce,YZbb),(Xi,Ce,YZbc), (Xi,Ce,YZbd),(Xi,Ce,YZbe),(Xi,Ce,zYZbf),(Xi,Ce,YZbg),(Xi,Ce,YZbh),(Xi,Ce,YZbi), (Xi,Ce,YZbj),(Xi,Ce,YZbk),(Xi,Ce,YZbl),(Xi,Ce,YZbm),(Xi,Ce,YZbn),(Xi,Ce,YZbo), (Xi,Ce,YZbp),(Xi,Ce,YZbq),(Xi,Ce,YZbr),(Xi,Ce,YZbs),(Xi,Ce,YZbt),(Xi,Ce,YZbu),(Xi, Ce,YZbv),(Xi,Ce,YZbw),(Xi,Ce,YZbx),(Xi,Ce,YZby),(Xi,Ce,YZbz),(Xi,Ce,YZca), (Xi,Ce,YZcb),(Xi,Ce,YZcc),(Xi,Ce,YZcd),(Xi,Ce,YZce),(Xi,Ce,YZcf),(Xi,Ce,YZcg),(Xi, Ce,YZch),(Xi,Ce,YZci),(Xi,Ce,YZcj),(Xi,Ce,YZck),(Xi,Ce,YZcl),(Xi,Ce,YZcm),(Xi, Ce,YZcn),(Xi,Ce,YZco),(Xi,Ce,YZcp),(Xi,Ce,YZcq),(Xi,Ce,YZcr),(Xi,Ce,YZcs),(Xi, Ce,YZct),(Xi,Ce,YZeu),(Xi,Ce,YZcv),(Xi,Ce,YZcw),(Xi,Ce,YZcx),(Xi,Ce,YZcy),(Xi, Ce,YZcz),(Xi,Ce,YZda),(Xi,Ce,YZdb),(Xi,Ce,YZdc),(Xi,Ce,YZdd),(Xi,Ce,YZde),(Xi, Ce,YZdf),(Xi,Ce,YZdg),(Xi,Ce,YZdh),(Xi,Ce,YZdi),(Xi,Ce,YZdj),(Xi,Ce,YZdk),(Xi, Ce,YZdl),(Xi,Ce,YZdm),(Xi,Ce,YZdn),(Xi,Ce,YZdo),(Xi,Ce,YZdp),(Xi,Ce,YZdq), (Xi,Ce,YZdr),(Xi,Ce,YZds),(Xi,Ce,Yzdt),(Xi,Ce,YZdu),(Xi,Ce,YZdv),(Xi,Ce,YZdw), (Xi,Ce,YZdx),(Xi,Ce,YZdy),(Xi,Ce,YZdz),(Xi,Ce,YZea),(Xi,Ce,YZeb),(Xi,Ce,YZec), (Xi,Ce,YZed),(Xi,Ce,YZee),(Xi,Ce,YZef),(Xi,Ce,YZeg),(Xi,Ce,YZeh),(Xi,Ce,YZei),(Xi, Ce,YZej),(Xi,Ce,YZek),(Xi,Ce,YZel),(Xi,Ce,YZem),(Xi,Ce,YZen),(Xi,Ce,YZeo),(Xi, Ce,YZep),(Xi,Ce,YZeq),(Xi,Ce,YZer),(Xi,Ce,YZes),(Xi,Ce,YZet),(Xi,Ce,YZeu),(Xi, Ce,YZev),(Xi,Ce,YZew),(Xi,Ce,YZex),(Xi,Ce,YZey),(Xi,Ce,YZez),(Xi,Ce,YZfa),(Xi, Ce,YZfb),(Xi,Ce,YZfc),(Xi,Ce,YZfd),(Xi,Ce,YZfe),(Xi,Ce,YZff),(Xi,Ce,YZfg),(Xi, Ce,YZfh),(Xi,Ce,YZfi),(Xi,Ce,YZfj),(Xi,Ce,YZfk),(Xi,Ce,YZfl),(Xi,Ce,YZfm),(Xi,Ce, YZfn),(Xi,Ce,YZfo),(Xi,Ce,YZfp),(Xi,Ce,YZfq),(Xi,Ce,YZfr),(Xi,Ce,YZfs),(Xi,Ce, YZft),(Xi,Ce,YZfu),(Xi,Ce,YZfv),(Xi,Ce,YZfw),(Xi,Ce,YZfx),(Xi,Ce,YZfy),(Xi,Ce,YZfz), (Xi,Ce,YZga),(Xi,Ce,YZgb),(Xi,Ce,YZgc),(Xi,Ce,YZgd),(Xi,Ce,YZge),(Xi,Ce,YZgf), (Xi,Ce,YZgg),(Xi,Ce,YZgh),(Xi,Ce,YZgi),(Xi,Ce,YZgji),(Xi,Ce,YZgk),(Xi,Ce,YZgl), (Xi,Ce,YZgm),(Xi,Ce,YZgn),(Xi,Ce,YZgo),(Xi,Ce,YZgp),(Xi,Ce,YZgq),(Xi,Ce, YZgr),(Xi,Ce,YZgs),(Xi,Ce,YZgt),(Xi,Cf,YZa),(Xi,Cf,YZb),(Xi,Cf,YZc),(Xi,Cf,YZd), (Xi,Cf,YZe),(Xi,Cf,YZf),(Xi,Cf,YZg),(Xi,Cf,YZh),(Xi,Cf,YZi),(Xi,Cf,YZj),(Xi,Cf,YZk), (Xi,Cf,YZl),(Xi,Cf,YZm),(Xi,Cf,YZn),(Xi,Cf,YZo),(Xi,Cf,YZp),(Xi,Cf,YZq),(Xi, Cf,YZr),(Xi,Cf,YZs),(Xi,Cf,YZt),(Xi,Cf,YZu),(Xi,Cf,YZv),(Xi,Cf,YZw),(Xi,Cf,YZx), (Xi,Cf,YZy),(Xi,Cf,YZz),(Xi,Cf,YZaa),(Xi,Cf,YZab),(Xi,Cf,YZac),(Xi,Cf,YZad),(Xi, Cf,YZae),(Xi,Cf,YZaf),(Xi,Cf,YZag),(Xi,Cf,YZah),(Xi,Cf,YZai),(Xi,Cf,YZaj),(Xi,Cf, YZak),(Xi,Cf,YZal),(Xi,Cf,YZam),(Xi,Cf,YZan),(Xi,Cf,YZao),(Xi,Cf,YZap),(Xi,Cf,YZaq), (Xi,Cf,YZar),(Xi,Cf,YZas),(Xi,Cf,YZat),(Xi,Cf,YZau),(Xi,Cf,YZav),(,Ni,Cf,YZaw), (Xi,Cf,YZax),(Xi,Cf,YZay),(Xi,Cf,YZaz),(Xi,Cf,YZba),(Xi,Cf,YZbb),(Xi,Cf,YZbc), (Xi,Cf,YZbd),(Xi,Cf,YZbe),(Xi,Cf,YZbf),(Xi,Cf,YZbg),(Xi,Cf,YZbh),(Xi,Cf,YZbi),(Xi, Cf,YZbj),(Xi,Cf,YZbk),(Xi,Cf,YZbl),(Xi,Cf,YZbm),(Xi,Cf,YZbn),(Xi,Cf,YZbo),(Xi, Cf,YZbp),(Xi,Cf,YZbq),(Xi,Cf,YZbr),(Xi,Cf,YZbs),(Xi,Cf,YZbt),(Xi,Cf,YZbu),(Xi, Cf,YZbv),(Xi,Cf,YZbw),(Xi,Cf,YZbx),(Xi,Cf,YZby),(Xi,Cf,YZbz),(Xi,Cf,YZca),(Xi, Cf,YZcb),(Xi,Cf,YZcc),(Xi,Cf,YZed),(Xi,Cf,YZce),(Xi,Cf,YZcf),(Xi,Cf,YZcg),(Xi,Cf, YZch),(Xi,Cf,YZci),(Xi,Cf,YZcj),(Xi,Cf,YZck),(Xi,Cf,YZcl),(Xi,Cf,YZcm),(Xi,Cf,YZcn), (Xi,Cf,YZco),(Xi,Cf,YZcp),(Xi,Cf,YZcq),(Xi,Cf,YZcr),(Xi,Cf,YZcs),(Xi,Cf,YZct), (Xi,Cf,YZcu),(Xi,Cf,YZcv),(Xi,Cf,YZcw),(Xi,Cf,YZcx),(Xi,Cf,YZcy),(Xi,Cf,YZcz), (Xi,Cf,YZda),(Xi,Cf,YZdb),(Xi,Cf,YZdc),(Xi,Cf,YZdd),(Xi,Cf,YZde),(Xi,Cf,YZdf),(Xi, Cf,YZdg),(Xi,Cf,YZdh),(Xi,Cf,YZdi),(Xi,Cf,YZdj),(Xi,Cf,YZdk),(Xi,Cf,YZdl),(Xi, Cf,YZdm),(Xi,Cf,YZdn),(Xi,Cf,YZdo),(Xi,Cf,YZdp),(Xi,Cf,YZdq),(Xi,Cf,YZdr),(Xi, Cf,YZds),(Xi,Cf,Yzdt),(Xi,Cf,YZdu),(Xi,Cf,YZdv),(Xi,Cf,YZdw),(Xi,Cf,YZdx),(Xi,Cf, YZdy),(Xi,Cf,YZdz),(Xi,Cf,YZea),(Xi,Cf,YZeb),(Xi,Cf,YZec),(Xi,Cf,YZed),(Xi,Cf, YZee),(Xi,Cf,YZef),(Xi,Cf,YZeg),(Xi,Cf,YZeh),(Xi,Cf,YZei),(Xi,Cf,YZej),(Xi,Cf,YZek), (Xi,Cf,YZel),(Xi,Cf,YZem),(Xi,Cf,yZen),(Xi,Cf,YZeo),(Xi,Cf,YZep),(Xi,Cf,YZeq), (Xi,Cf,YZer),(Xi,Cf,YZes),(Xi,Cf,YZet),(Xi,Cf,YZeu),(Xi,Cf,YZev),(Xi,Cf,YZew), (Xi,Cf,YZex),(Xi,Cf,YZey),(Xi,Cf,YZez),(Xi,Cf,YZfa),(Xi,Cf,YZfb),(Xi,Cf,YZfc),(Xi, Cf,YZfd),(Xi,Cf,YZfe),(Xi,Cf,YZff),(Xi,Cf,YZfg),(Xi,Cf,YZfh),(Xi,Cf,YZfi),(Xi,Cf,YZfj), (Xi,Cf,YZfk),(Xi,Cf,YZfl),(Xi,Cf,YZfm),(Xi,Cf,YZfn),(Xi,Cf,YZfo),(Xi,Cf,YZfp), (Xi,Cf,YZfq),(Xi,Cf,YZfr),(Xi,Cf,YZfs),(Xi,Cf,YZft),(Xi,Cf,YZfu),(Xi,Cf,YZfv),(Xi, Cf,YZfw),(Xi,Cf,YZfx),(Xi,Cf,YZfy),(Xi,Cf,YZfz),(Xi,Cf,YZga),(Xi,Cf,YZgb),(Xi, Cf,YZgc),(Xi,Cf,YZgd),(Xi,Cf,YZge),(Xi,Cf,YZgf),(Xi,Cf,YZgg),(Xi,Cf,YZgh),(Xi, Cf,YZgi),(Xi,Cf,YZgj),(Xi,Cf,YZgk),(Xi,Cf,YZgl),(Xi,Cf,YZgm),(Xi,Cf,YZgn),(Xi,Cf, YZgo),(Xi,Cf,YZgp),(Xi,Cf,YZgq),(Xi,Cf,YZgr),(Xi,Cf,YZgs),(Xi,Cf,YZgt), (Xj,Ca,YZa),(Xj,Ca,YZb),(Xj,Ca,YZc),(Xj,Ca,YZd),(Xj,Ca,YZe),(Xj,Ca,YZf),(Xj,Ca, YZg),(Xj,Ca,YZh),(Xj,Ca,YZi),(Xj,Ca,YZj),(Xj,Ca,YZk),(Xj,Ca,YZl),(Xj,Ca,YZm),(Xj, Ca,YZn),(Xj,Ca,YZo),(Xj,Ca,YZp),(Xj,Ca,YZq),(Xj,Ca,YZr),(Xj,Ca,YZs),(Xj,Ca, YZt),(Xj,Ca,YZu),(Xj,Ca,YZv),(Xj,Ca,YZw),(Xj,Ca,YZx),(Xj,Ca,YZy),(Xj,Ca,YZz),(Xj, Ca,YZaa),(Xj,Ca,YZab),(Xj,Ca,YZac),(Xj,Ca,YZad),(Xj,Ca,YZae),(Xj,Ca,YZaf),(Xj, Ca,YZag),(Xj,Ca,YZah),(Xj,Ca,YZai),(Xj,Ca,YZaj),(Xj,Ca,YZak),(Xj,Ca,YZal),(Xj, Ca,YZam),(Xj,Ca,YZan),(Xj,Ca,YZao),(Xj,Ca,YZap),(Xj,Ca,YZaq),(Xj,Ca,YZar),(Xj, Ca,YZas),(Xj,Ca,YZat),(Xj,Ca,YZau),(Xj,Ca,YZav),(Xj,Ca,YZaw),(Xj,Ca,YZax),(Xj, Ca,YZay),(Xj,Ca,YZaz),(Xj,Ca,YZba),(Xj,Ca,YZbb),(Xj,Ca,YZbc),(Xj,Ca,YZbd),(Xj, Ca,YZbe),(Xj,Ca,YZbf),(Xj,Ca,YZbg),(Xj,Ca,YZbh),(Xj,Ca,YZbi),(Xj,Ca,YZbj),(Xj, Ca,YZbk),(Xj,Ca,YZbl),(Xj,Ca,YZbm),(Xj,Ca,YZbn),(Xj,Ca,YZbo),(Xj,Ca,YZbp),(Xj, Ca,YZbq),(Xj,Ca,YZbr),(Xj,Ca,YZbs),(Xj,Ca,YZbt),(Xj,Ca,YZbu),(Xj,Ca,YZbv),(Xj, Ca,YZbw),(Xj,Ca,YZbx),(Xj,Ca,YZby),(Xj,Ca,YZbz),(Xj,Ca,YZca),(Xj,Ca,YZcb),(Xj, Ca,YZcc),(Xj,Ca,YZcd),(Xj,Ca,YZce),(Xj,Ca,YZcf),(Xj,Ca,YZcg),(Xj,Ca,YZch),(Xj, Ca,YZci),(Xj,Ca,YZcj),(Xj,Ca,YZck),(Xj,Ca,YZcl),(Xj,Ca,YZcm),(Xj,Ca,YZcn),(Xj, Ca,YZco),(Xj,Ca,YZcp),(Xj,Ca,YZcq),(Xj,Ca,YZcr),(Xj,Ca,YZcs),(Xj,Ca,YZct),(Xj,Ca, YZcu),(Xj,Ca,YZcv),(Xj,Ca,YZcw),(Xj,Ca,YZcx),(Xj,Ca,YZcy),(Xj,Ca,YZcz),(Xj,Ca, YZda),(Xj,Ca,YZdb),(Xj,Ca,YZdc),(Xj,Ca,YZdd),(Xj,Ca,YZde),(Xj,Ca,YZdf),(Xj,Ca, YZdg),(Xj,Ca,YZdh),(Xj,Ca,YZdi),(Xj,Ca,YZdj),(Xj,Ca,YZdk),(Xj,Ca,YZdl),(Xj,Ca, YZdm),(Xj,Ca,YZdn),(Xj,Ca,YZdo),(Xj,Ca,YZdp),(Xj,Ca,YZdq),(Xj,Ca,YZdr),(Xj,Ca, YZds),(Xj,Ca,Yzdt),(Xj,Ca,YZdu),(Xj,Ca,YZdv),(Xj,Ca,YZdw),(Xj,Ca,YZdx),(Xj,Ca, YZdy),(Xj,Ca,YZdz),(Xj,Ca,YZea),(Xj,Ca,YZeb),(Xj,Ca,YZec),(Xj,Ca,YZed),(Xj,Ca, YZee),(Xj,Ca,YZef),(Xj,Ca,YZeg),(Xj,Ca,YZeh),(Xj,Ca,YZei),(Xj,Ca,YZej),(Xj,Ca, YZek),(Xj,Ca,YZel),(Xj,Ca,YZem),(Xj,Ca,YZen),(Xj Ca,YZeo),(Xj,Ca,YZep),(Xj,Ca, YZeq),(Xj,Ca,YZer),(Xj,Ca,YZes),(Xj,Ca,YZet),(Xj,Ca,YZeu),(Xj,Ca,YZev),(Xj,Ca, YZew),(Xj,Ca,YZex),(Xj,Ca,YZey),(Xj,Ca,YZez),(Xj,Ca,YZfa),(Xj,Ca,YZfb),(Xj,Ca, YZfc),(Xj,Ca,YZfd),(Xj,Ca,YZfe),(Xj,Ca,YZff),(Xj,Ca,YZfg),(Xj,Ca,YZfh),(Xj,Ca,YZfi), (Xj,Ca,YZij),(Xj,Ca,YZfk),(Xj,Ca,YZfl),(Xj,Ca,YZfm),(Xj,Ca,YZfn),(Xj,Ca,YZfo), (Xj,Ca,YZfp),(Xj,Ca,YZfq),(Xj,Ca,YZfr),(Xj,Ca,YZfs),(Xj,Ca,YZft),(Xj,Ca,YZfu), (Xj,Ca,YZfv),(Xj,Ca,YZfw),(Xj,Ca,YZfx),(Xj,Ca,YZfy),(Xj,Ca,YZfz),(Xj,Ca,YZga),(Xj, Ca,YZgb),(Xj,Ca,YZgc),(Xj,Ca,YZgd),(Xj,Ca,YZge),(Xj,Ca,YZgf),(Xj,Ca,YZgg),(Xj, Ca,YZgh),(Xj,Ca,YZgi),(Xj,Ca,YZgj),(Xj,Ca,YZgk),(Xj,Ca,YZgl),(Xj,Ca,YZgm),(Xj, Ca,YZgn),(Xj,Ca,YZgo),(Xj,Ca,YZgp),(Xj,Ca,YZgq),(Xj,Ca,YZgr),(Xj,Ca,YZgs),(Xj, Ca,YZgt),(Xj,Cb,YZa),(Xj,Cb,YZb),(Xj,Cb,YZc),(Xj,Cb,YZd),(Xj,Cb,YZe),(Xj,Cb, YZf),(Xj,Cb,YZg),(Xj,Cb,YZh),(Xj,Cb,YZi),(Xj,Cb,YZj),(Xj,Cb,YZk),(Xj,Cb,YZl),(Xj, Cb,YZm),(Xj,Cb,YZn),(Xj,Cb,YZo),(Xj,Cb,YZp),(Xj,Cb,YZq),(Xj,Cb,YZr),(Xj,Cb, YZs),(Xj,Cb,YZt),(Xj,Cb,YZu),(Xj Cb,YZv),(Xj,Cb,YZw),(Xj,Cb,YZx),(Xj,Cb,YZy), Xj,Cb,YZz),(Xj,Cb,YZaa),(Xj,Cb,YZab),(Xj,Cb,YZac),(Xj,Cb,YZad),(Xj,Cb,YZae), (Xj,Cb,YZaf),(Xj,Cb,YZag),(Xj,Cb,YZah),(Xj,Cb,YZai),(Xj,Cb,YZaj),(Xj,Cb,YZak), (Xj,Cb,YZal),(Xj,Cb,YZam),(Xj,Cb,YZan),(Xj,Cb,YZao),(Xj,Cb,YZap),(Xj,Cb,YZaq), (Xj,Cb,YZar),(Xj,Cb,YZas),(Xj,Cb,YZat),(Xj,Cb,YZau),(Xj,Cb,YZav),(Xj,Cb,YZaw), (Xj,Cb,YZax),(Xj,Cb,YZay),(Xj,Cb,YZaz),(Xj,Cb,YZba),(Xj,Cb,YZbb),(Xj,Cb,YZbc), (Xj,Cb,YZbd),(Xj,Cb,YZbe),(Xj,Cb,YZbf),(Xj,Cb,YZbg),(Xj,Cb,YZbh),(Xj,Cb,YZbi), (Xj,Cb,YZbj),(Xj,Cb,YZbk),(Xj,Cb,YZbl),(Xj,Cb,YZbm),(Xj,Cb,YZbn),(Xj,Cb,YZbo), (Xj,Cb,YZbp),(Xj,Cb,YZbq),(Xj,Cb,YZbr),(Xj,Cb,YZbs),(Xj,Cb,YZbt),(Xj,Cb,YZbu), (Xj,Cb,YZbv),(Xj,Cb,YZbw),(Xj,Cb,YZbx),(Xj,Cb,YZby),(Xj,Cb,YZbz),(Xj,Cb, YZca),(Xj,Cb,YZcb),(Xj,Cb,YZcc),(Xj,Cb,YZcd),(Xj,Cb,YZce),(Xj,Cb,YZcf),(Xj,Cb, YZcg),(Xj,Cb,YZch),(Xj,Cb,YZci),(Xj,Cb,YZcj),(Xj,Cb,YZck),(Xj,Cb,YZcl),(Xj,Cb, YZcm),(Xj,Cb,YZcn),(Xj,Cb,YZco),(Xj,Cb,YZcp),(Xj,Cb,YZcq),(Xj,Cb,YZcr),(Xj,Cb, YZcs),(Xj,Cb,YZct),(Xj,Cb,YZcu),(Xj,Cb,YZcv),(Xj,Cb,YZcw),(Xj,Cb,YZcx),(Xj, Cb,YZcy),(Xj,Cb,YZcz),(Xj,Cb,YZda),(Xj,Cb,YZdb),(Xj,Cb,YZdc),(Xj,Cb,YZdd),(Xj, Cb,YZde),(Xj,Cb,YZdf),(Xj,Cb,YZdg),(Xj,Cb,YZdh),(Xj,Cb,YZdi),(Xj,Cb,YZdj),(Xj, Cb,YZdk),(Xj,Cb,YZdl),(Xj,Cb,YZdm),(Xj,Cb,YZdn),(Xj,Cb,YZdo),(Xj,Cb,YZdp),(Xj, Cb,YZdq),(Xj,Cb,YZdr),(Xj,Cb,YZds),(Xj,Cb,Yzdt),(Xj,Cb,YZdu),(Xj,Cb,YZdv),(Xj, Cb,YZdw),(Xj,Cb,YZdx),(Xj,Cb,YZdy),(Xj,Cb,YZdz),(Xj,Cb,YZea),(Xj,Cb,YZeb), (Xj,Cb,YZec),(Xj,Cb,YZed),(Xj,Cb,YZee),(Xj,Cb,YZef),(Xj,Cb,YZeg),(Xj,Cb,YZeh), (Xj,Cb,YZei),(Xj,Cb,YZej),(Xj,Cb,YZek),(Xj,Cb,YZel),(Xj,Cb,YZem),(Xj,Cb,YZen), (Xj,Cb,YZeo),(Xj,Cb,YZep),(Xj,Cb,YZeq),(Xj,Cb,YZer),(Xj,Cb,YZes),(Xj,Cb,YZet), (Xj,Cb,YZeu),(Xj,Cb,YZev),(Xj,Cb,YZew),(Xj,Cb,YZex),(Xj,Cb,YZey),(Xj,Cb,YZez), (Xj,Cb,YZfa),(Xj,Cb,YZfb),(Xj,Cb,YZfc),(Xj,Cb,YZfd),(Xj,Cb,YZfe),(Xj,Cb,YZff), (Xj,Cb,YZfg),(Xj,Cb,YZfh),(Xj,Cb,YZfi),(Xj,Cb,YZfj),(Xj,Cb,YZfk),(Xj,Cb,YZfl),(Xj, Cb,YZfm),(Xj,Cb,YZfn),(Xj,Cb,YZfo),(Xj,Cb,YZfp),(Xj,Cb,YZfq),(Xj,Cb,YZfr),(Xj, Cb,YZfs),(Xj,Cb,YZft),(Xj,Cb,YZfu),(Xj,Cb,YZfv),(Xj,Cb,YZfw),(Xj,Cb,YZfx),(Xj, Cb,YZfy),(Xj,Cb,YZfz),(Xj,Cb,YZga),(Xj,Cb,YZgb),(Xj,Cb,YZgc),(Xj,Cb,YZgd),(Xj, Cb,YZge),(Xj,Cb,YZgf),(Xj,Cb,YZgg),(Xj,Cb,YZgh),(Xj,Cb,YZgi),(Xj,Cb,YZgj),(Xj, Cb,YZgk),(Xj,Cb,YZgl),(Xj,Cb,YZgm),(Xj,Cb,YZgn),(Xj,Cb,YZgo),(Xj,Cb,YZgp),(Xj, Cb,YZgq),(Xj,Cb,YZgr),(Xj,Cb,YZgs),(Xj,Cb,YZgt),(Xj,Cc,YZa),(Xj,Cc,YZb),(Xj, Cc,YZc),(Xj,Cc,YZd),(Xj,Cc,YZe),(Xj,Cc,YZf),(Xj,Cc,YZg),(Xj,Cc,YZh),(Xj,Cc,YZi), (Xj,Cc,YZj),(Xj,Cc,YZk),(Xj,Cc,YZl),(Xj,Cc,YZm),(Xj,Cc,YZn),(Xj,Cc,YZo),(Xj, Cc,YZp),(Xj,Cc,YZq),(Xj,Cc,YZr),(Xj,Cc,YZs),(Xj,Cc,YZt),(Xj,Cc,YZu),(Xj,Cc,YZv), (Xj,Cc,YZw),(Xj,Cc,YZx),(Xj,Cc,YZy),(Xj,Cc,YZz),(Xj,Cc,YZaa),(Xj,Cc,YZab),(Xj, Cc,YZac),(Xj,Cc,YZad),(Xj,Cc,YZae),(Xj,Cc,YZaf),(Xj,Cc,YZag),(Xj,Cc,YZah),(Xj, Cc,YZai),(Xj,Cc,YZaj),(Xj,Cc,YZak),(Xj,Cc,YZal),(Xj,Cc,YZam),(Xj,Cc,YZan),(Xj, Cc,YZao),(Xj,Cc,YZap),(Xj,Cc,YZaq),(Xj,Cc,YZar),(Xj,Cc,YZas),(Xj,Cc,YZat),(Xj,Cc, YZau),(Xj,Cc,YZav),(Xj,Cc,YZaw),(Xj,Cc,YZax),(Xj,Cc,YZay),(Xj,Cc,YZaz),(Xj,Cc, YZba),(Xj,Cc,YZbb),(Xj,Cc,YZbc),(Xj,Cc,YZbd),(Xj,Cc,YZbe),(Xj,Cc,YZbf),(Xj,Cc, YZbg),(Xj,Cc,YZbh),(Xj,Cc,YZbi),(Xj,Cc,YZbj),(Xj,Cc,YZbk),(Xj,Cc,YZbl),(Xj,Cc, YZbm),(Xj,Cc,YZbn),(Xj,Cc,YZbo),(Xj,Cc,YZbp),(Xj,Cc,YZbq),(Xj,Cc,YZbr),(Xj,Cc, YZbs),(Xj,Cc,YZbt),(Xj,Cc,YZbu),(Xj,Cc,YZbv),(Xj,Cc,YZbw),(Xj,Cc,YZbx),(Xj, Cc,YZby),(Xj,Cc,YZbz),(Xj,Cc,YZca),(Xj,Cc,YZcb),(Xj,Cc,YZcc),(Xj,Cc,YZcd),(Xj, Cc,YZce),(Xj,Cc,YZcf),(Xj,Cc,YZcg),(Xj,Cc,YZch),(Xj,Cc,YZci),(Xj,Cc,YZcj),(Xj,Cc, YZck),(Xj,Cc,YZcl),(Xj,Cc,YZcm),(Xj,Cc,YZcn),(Xj,Cc,YZco),(Xj,Cc,YZcp),(Xj,Cc, YZcq),(Xj,Cc,YZcr),(Xj,Cc,YZcs),(Xj,Cc,YZct),(Xj,Cc,YZcu),(Xj,Cc,YZcv),(Xj,Cc, YZcw),(Xj,Cc,YZcx),(Xj,Cc,YZcy),(Xj,Cc,YZcz),(Xj,Cc,YZda),(Xj,Cc,YZdb),(Xj,Cc, YZdc),(Xj,Cc,YZdd),(Xj,Cc,YZde),(Xj,Cc,YZdf),(Xj,Cc,YZdg),(Xj,Cc,YZdh),(Xj,Cc, YZdi),(Xj,Cc,YZdj),(Xj,Cc,YZdk),(Xj,Cc,YZdl),(Xj,Cc,YZdm),(Xj,Cc,YZdn),(Xj,Cc, YZdo),(Xj,Cc,YZdp),(Xj,Cc,YZdq),(Xj,Cc,YZdr),(Xj,Cc,YZds),(Xj,Cc,Yzdt),(Xj,Cc, YZdu),(Xj,Cc,YZdv),(Xj,Cc,YZdw),(Xj,Cc,YZdx),(Xj,Cc,YZdy),(Xj,Cc,YZdz),(Xj,Cc, YZea),(Xj,Cc,YZeb),(Xj,Cc,YZec),(Xj,Cc,YZed),(Xj,Cc,YZee),(Xj,Cc,YZef),(Xj,Cc, YZeg),(Xj,Cc,YZeh),(Xj,Cc,YZei),(Xj,Cc,YZej),(Xj,Cc,YZek),(Xj,Cc,YZel),(Xj,Cc, YZem),(Xj,Cc,YZen),(Xj,Cc,YZeo),(Xj,Cc,YZep),(Xj,Cc,YZeq),(Xj,Cc,YZer),(Xj,Cc, YZes),(Xj,Cc,YZet),(Xj,Cc,YZeu),(Xj,Cc,YZev),(Xj,Cc,YZew),(Xj,Cc,YZex),(Xj,Cc, YZey),(Xj,Cc,YZez),(Xj,Cc,YZfa),(Xj,Cc,YZfb),(Xj,Cc,YZfc),(Xj,Cc,YZfd),(Xj,Cc,YZfe), (Xj,Cc,YZff),(Xj,Cc,YZfg),(Xj,Cc,YZfh),(Xj,Cc,YZfi),(Xj,Cc,YZfj),(Xj,Cc,YZfk), (Xj,Cc,YZfl),(Xj,Cc,YZfm),(Xj,Cc,YZfn),(Xj,Cc,YZfo),(Xj,Cc,YZfp),(Xj,Cc,YZfq), (Xj,Cc,YZfr),(Xj,Cc,YZfs),(Xj,Cc,YZft),(Xj,Cc,YZfu),(Xj,Cc,YZfv),(Xj,Cc,YZfw),(Xj, Cc,YZfx),(Xj,Cc,YZfy),(Xj,Cc,YZfz),(Xj,Cc,YZga),(Xj,Cc,YZgb),(Xj,Cc,YZgc),(Xj, Cc,YZgd),(Xj,Cc,YZge),(Xj,Cc,YZgf),(Xj,Cc,YZgg),(Xj,Cc,YZgh),(Xj,Cc,YZgi),(Xj, Cc,YZgj),(Xj,Cc,YZgk),(Xj,Cc,YZgl),(Xj,Cc,YZgm),(Xj,Cc,YZgn),(Xj,Cc,YZgo),(Xj, Cc,YZgp),(Xj,Cc,YZgq),(Xj,Cc,YZgr),(Xj,Cc,YZgs),(Xj,Cc,YZgt),(Xj,Cd,YZa),(Xj,Cd, YZb),(Xj,Cd,YZc),(Xj,Cd,YZd),(Xj,Cd,YZe),(Xj,Cd,YZf),(Xj,Cd,YZg),(Xj,Cd,YZh), (Xj,Cd,YZi),(Xj,Cd,YZj),(Xj,Cd,YZk),(Xj,Cd,YZl),(Xj,Cd,YZm),(Xj,Cd,YZn),(Xj,Cd, YZo),(Xj,Cd,YZp),(Xj,Cd,YZq),(Xj,Cd,YZr),(Xj,Cd,YZs),(Xj,Cd,YZt),(Xj,Cd,YZu), (Xj,Cd,YZv),(Xj,Cd,YZw),(Xj,Cd,YZx),(Xj,Cd,YZy),(Xj,Cd,YZz),(Xj,Cd,YZaa),(Xj, Cd,YZab),(Xj,Cd,YZac),(Xj,Cd,YZad),(Xj,Cd,YZae),(Xj,Cd,YZaf),(Xj,Cd,YZag),(Xj, Cd,YZah),(Xj,Cd,YZai),(Xj,Cd,YZaj),(Xj,Cd,YZak),(Xj,Cd,YZal),(Xj,Cd,YZam),(Xj, Cd,YZan),(Xj,Cd,YZao),(Xj,Cd,YZap),(Xj,Cd,YZaq),(Xj,Cd,YZar),(Xj,Cd,YZas),(Xj, Cd,YZat),(Xj,Cd,YZau),(Xj,Cd,YZav),(Xj,Cd,YZaw),(Xj,Cd,YZax),(Xj,Cd,YZay),(Xj, Cd,YZaz),(Xj,Cd,YZba),(Xj,Cd,YZbb),(Xj,Cd,YZbc),(Xj,Cd,YZbd),(Xj,Cd,YZbe),(Xj, Cd,YZbf),(Xj,Cd,YZbg),(Xj,Cd,YZbh),(Xj,Cd,YZbi),(Xj,Cd,YZbj),(Xj,Cd,YZbk),(Xj, Cd,YZbl),(Xj,Cd,YZbm),(Xj,Cd,YZbn),(Xj,Cd,YZbo),(Xj,Cd,YZbp),(Xj,Cd,YZbq),(Xj, Cd,YZbr),(Xj,Cd,YZbs),(Xj,Cd,YZbt),(Xj,Cd,YZbu),(Xj,Cd,YZbv),(Xj,Cd,YZbw), (Xj,Cd,YZbx),(Xj,Cd,YZby),(Xj,Cd,YZbz),(Xj,Cd,YZca),(Xj,Cd,YZcb),(Xj,Cd,YZcc), (Xj,Cd,YZcd),(Xj,Cd,YZce),(Xj,Cd,YZcf),(Xj,Cd,YZcg),(Xj,Cd,YZch),(Xj,Cd,YZci), (Xj,Cd,YZcj),(Xj,Cd,YZck),(Xj,Cd,YZcl),(Xj,Cd,YZcm),(Xj,Cd,YZcn),(Xj,Cd,YZco), (Xj,Cd,YZcp),(Xj,Cd,YZcq),(Xj,Cd,YZcr),(Xj,Cd,YZcs),(Xj,Cd,YZct),(Xj,Cd,YZcu),(Xj, Cd,YZcv),(Xj,Cd,YZcw),(Xj,Cd,YZcx),(Xj,Cd,YZcy),(Xj,Cd,YZcz),(Xj,Cd,YZda), (Xj,Cd,YZdb),(Xj,Cd,YZdc),(Xj,Cd,YZdd),(Xj,Cd,YZde),(Xj,Cd,YZdf),(Xj,Cd,YZdg), (Xj,Cd,YZdh),(Xj,Cd,YZdi),(Xj,Cd,YZdj),(Xj,Cd,YZdk),(Xj,Cd,YZdl),(Xj,Cd,YZdm), (Xj,Qd,YZdn),(Xj,Cd,YZdo),(Xj,Cd,YZdp),(Xj,Cd,YZdq),(Xj,Cd,YZdr),(Xj,Cd,YZds), (Xj,Cd,Yzdt),(Xj,Cd,YZdu),(Xj,Cd,YZdv),(Xj,Cd,YZdw),(Xj,Cd,YZdx),(Xj,Cd,YZdy), (Xj,Cd,YZdz),(Xj,Cd,YZea),(Xj,Cd,YZeb),(Xj,Cd,YZec),(Xj,Cd,YZed),(Xj,Cd,YZee), (Xj,Cd,YZef),(Xj,Cd,YZeg),(Xj,Cd,YZeh),(Xj,Cd,YZei),(Xj,Cd,YZej),(Xj,Cd,YZek), (Xj,Cd,YZel),(Xj,Cd,YZem),(Xj,Cd,YZen),(Xj,Cd,YZeo),(Xj,Cd,YZep),(Xj,Cd, YZeq),(Xj,Cd,YZer),(Xj,Cd,YZes),(Xj,Cd,YZet),(Xj,Cd,YZeu),(Xj,Cd,YZev),(Xj,Cd, YZew),(Xj,Cd,YZex),(Xj,Cd,YZey),(Xj,Cd,YZez),(Xj,Cd,YZfa),(Xj,Cd,YZfb),(Xj,Cd, YZfc),(Xj,Cd,YZfd),(Xj,Cd,YZfe),(Xj,Cd,YZff),(Xj,Cd,YZfg),(Xj,Cd,YZfh),(Xj,Cd, YZfi),(Xj,Cd,YZj),(Xj,Cd,YZfk),(Xj,Cd,YZfl),(Xj,Cd,YZfm),(Xj,Cd,YZfn),(Xj,Cd,YZfo), (Xj,Cd,YZfp),(Xj,Cd,YZfq),(Xj,Cd,YZfr),(Xj,Cd,YZfs),(Xj,Cd,YZft),(Xi,Cd,YZfu), (Xj,Cd,YZfv),(Xj,Cd,YZfw),(Xj,Cd,YZfx),(Xj,Cd,YZfy),(Xj,Cd,YZfz),(Xj,Cd,YZga), (Xj,Cd,YZgb),(Xj,Cd,YZgc),(Xj,Cd,YZgd),(Xj,Cd,YZge),(Xj,Cd,YZgf),(Xj,Cd,YZgg), (Xj,Cd,YZgh),(Xj,Cd,YZgi),(Xj,Cd,YZgj),(Xj,Cd,YZgk),(Xj,Cd,YZgl),(Xj,Cd,YZgm), (Xj,Cd,YZgn),(Xj,Cd,YZgo),(Xj,Cd,YZgp),(Xj,Cd,YZgq),(Xj,Cd,YZgr),(Xj,Cd,YZgs), (Xj,Cd,YZgt),(Xj,Ce,YZa),(Xj,Ce,YZb),(Xj,Ce,YZc),(Xj,Ce,YZd),(Xj,Ce,YZe),(Xj, Ce,YZf),(Xj,Ce,YZg),(Xj,Ce,YZh),(Xj,Ce,YZi),(Xj,Ce,YZj),(Xj,Ce,YZk),(Xj,Ce,YZl), (Xj,Ce,YZm),(Xj,Ce,YZn),(Xj,Ce,YZo),(Xj,Ce,YZp),(Xj,Ce,YZq),(Xj,Ce,YZr),(Xj, Ce,YZs),(Xj,Ce,YZt),(Xj,Ce,YZu),(Xj,Ce,YZv),(Xj,Ce,YZw),(Xj,Ce,YZx),(Xj,Ce,YZy), (Xj,Ce,YZz),(Xj,Ce,YZaa),(Xj,Ce,YZab),(Xj,Ce,YZac),(Xj,Ce,YZad),(Xj,Ce,YZae), (Xj,Ce,YZaf),(Xj,Ce,YZag),(Xj,Ce,YZah),(Xj,Ce,YZai),(Xj,Ce,YZaj),(Xj,Ce,YZak), (Xj,Ce,YZal),(Xj,Ce,YZam),(Xj,Ce,YZan),(Xj,Ce,YZao),(Xj,Ce,YZap),(Xj,Ce,YZaq), (Xj,Ce,YZar),(Xj,Ce,YZas),(Xj,Ce,YZat),(Xj,Ce,YZau),(Xj,Ce,YZav),(Xj,Ce,YZaw), (Xj,Ce,YZax),(Xj,Ce,YZay),(Xj,Ce,YZaz),(Xj,Ce,YZba),(Xj,Ce,YZbb),(Xj,Ce,YZbc), (Xj,Ce,YZbd),(Xj,Ce,YZbe),(Xj,Ce,YZbf),(Xj,Ce,YZbg),(Xj,Ce,YZbh),(Xj,Ce,YZbi), (Xj,Ce,YZbj),(Xj,Ce,YZbk),(Xj,Ce,YZbl),(Xj,Ce,YZbm),(Xj,Ce,YZbn),(Xj,Ce,YZbo), (Xj,Ce,YZbp),(Xj,Ce,YZbq),(Xj,Ce,YZbr),(Xj,Ce,YZbs),(Xj,Ce,YZbt),(Xj,Ce,YZbu),(Xj, Ce,YZbv),(Xj,Ce,YZbw),(Xj,Ce,YZbx),(Xj,Ce,YZby),(Xj,Ce,YZbz),(Xj,Ce,YZca), (Xj,Ce,YZcb),(Xj,Ce,YZcc),(Xj,Ce,YZcd),(Xj,Ce,YZce),(Xj,Ce,YZcf),(Xj,Ce,YZcg),(Xj, Ce,YZch),(Xj,Ce,YZci),(Xj,Ce,YZcj),(Xj,Ce,YZck),(Xj,Ce,YZcl),(Xj,Ce,YZcm),(Xj, Ce,YZcn),(Xj,Ce,YZco),(Xj,Ce,YZcp),(Xj,Ce,YZcq),(Xj,Ce,YZcr),(Xj,Ce,YZcs),(Xj, Ce,YZct),(Xj,Ce,YZcu),(Xj,Ce,YZcv),(Xj,Ce,YZcw),(Xj,Ce,YZcx),(Xj,Ce,YZcy),(Xj, Ce,YZcz),(Xj,Ce,YZda),(Xj,Ce,YZdb),(Xj,Ce,YZdc),(Xj,Ce,YZdd),(Xj,Ce,YZde),(Xj, Ce,YZdf),(Xj,Ce,YZdg),(Xj,Ce,YZdh),(Xj,Ce,YZdi),(Xj,Ce,YZdj),(Xj,Ce,YZdk),(Xj, Ce,YZdl),(Xj,Ce,YZdm),(Xj,Ce,YZdn),(Xj,Ce,YZdo),(Xj,Ce,YZdp),(Xj,Ce,YZdq), (Xj,Ce,YZdr),(Xj,Ce,YZds),(Xj,Ce,Yzdt),(Xj,Ce,YZdu),(Xj,Ce,YZdv),(Xj,Ce,YZdw), (Xj,Ce,YZdx),(Xj,Ce,YZdy),(Xj,Ce,YZdz),(Xj,Ce,YZea),(Xj,Ce,YZeb),(Xj,Ce,YZec), (Xj,Ce,YZed);(Xj,Ce,YZee),(Xj,Ce,YZef),(Xj,Ce,YZeg),(Xj,Ce,YZeh),(Xj,Ce,YZei),(Xj, Ce,YZej),(Xj,Ce,YZek),(Xj,Ce,YZel),(Xj,Ce,YZem),(Xj,Ce,YZen),(Xj,Ce,YZeo),(Xj, Ce,YZep),(Xj,Ce,YZeq),(Xj,Ce,YZer),(Xj,Ce,YZes),(Xj,Ce,YZet),(Xj,Ce,YZeu),(Xj, Ce,YZev),(Xj,Ce,YZew),(Xj,Ce,YZex),(Xj,Ce,YZey),(Xj,Ce,YZez),(Xj,Ce,YZfa),(Xj, Ce,YZfb),(Xj,Ce,YZfc),(Xj,Ce,YZfd),(Xj,Ce,YZfe),(Xj,Ce,YZff),(Xj,Ce,YZfg),(Xj, Ce,YZfh),(Xj,Ce,YZfi),(Xj,Ce,YZfj),(Xj,Ce,YZfk),(Xj,Ce,YZfl),(Xj,Ce,YZfm),(Xj,Ce, YZfn),(Xj,Ce,YZfo),(Xj,Ce,YZfp),(Xj,Ce,YZfq),(Xj,Ce,YZfr),(Xj,Ce,YZfs),(Xj,Ce, YZft),(Xj,Ce,YZfu),(Xj,Ce,YZfv),(Xj,Ce,YZfw),(Xj,Ce,YZfx),(Xj,Ce,YZfy),(Xj,Ce,YZfz), (Xj,Ce,YZga),(Xj,Ce,YZgb),(Xj,Ce,YZgc),(Xj,Ce,YZgd),(Xj,Ce,YZge),(Xj,Ce,YZgf), (Xj,Ce,YZgg),(Xj,Ce,YZgh),(Xj,Ce,YZgi),(Xj,Ce,YZgj),(Xj,Ce,YZgk),(Xj,Ce,YZgl), (Xj,Ce,YZgm),(Xj,Ce,YZgn),(Xj,Ce,YZgo),(Xj,Ce,YZgp),(Xj,Ce,YZgq),(Xj,Ce, YZgr),(Xj,Ce,YZgs),(Xj,Ce,YZgt),(Xj,Cf,YZa),(Xj,Cf,YZb),(Xj,Cf,YZc),(Xj,Cf,YZd), (Xj,Cf,YZe),(Xj,Cf,YZf),(Xj,Cf,YZg),(Xj,Cf,YZh),(Xj,Cf,YZi),(Xj,Cf,YZj),(Xj,Cf,YZk), (Xj,Cf,YZl),(Xj,Cf,YZm),(Xj,Cf,YZn),(Xj,Cf,YZo),(Xj,Cf,YZp),(Xj,Cf,YZq),(Xj, Cf,YZr),(Xj,Cf,YZs),(Xj,Cf,YZt),(Xj,Cf,YZu),(Xj,Cf,YZv),(Xj,Cf,YZw),(Xj,Cf,YZx), (Xj,Cf,YZy),(Xj,Cf,YZz),(Xj,Cf,YZaa),(Xj,Cf,YZab),(Xj,Cf,YZac),(Xj,Cf,YZad),(Xj, Cf,YZae),(Xj,Cf,YZaf),(Xj,Cf,YZag),(Xj,Cf,YZah),(Xj,Cf,YZai),(Xj,Cf,YZaj),(Xj,Cf, YZak),(Xj,Cf,YZal),(Xj,Cf,YZam),(Xj,Cf,YZan),(Xj,Cf,YZao),(ij,Cf,YZap),(Xj,Cf,YZaq), (Xj,Cf,YZar),(Xj,Cf,YZas),(Xj,Cf,YZat),(Xj,Cf,YZau),(Xj,Cf,YZav),(Xj,Cf,YZaw), (Xj,Cf,YZax),(Xj,Cf,YZay),(Xj,Cf,YZaz),(Xj,Cf,YZba),(Xj,Cf,YZbb),(Xj,Cf,YZbc), (Xj,Cf,YZbd),(Xj,Cf,YZbe),(Xj,Cf,YZbf),(Xj,Cf,YZbg),(Xj,Cf,YZbh),(Xj,Cf,YZbi),(Xj, Cf,YZbj),(Xj,CfYZbk),(Xj,Cf,YZbl),(Xj,Cf,YZbm),(Xj,Cf,YZbn),(Xj,Cf,YZbo),(Xj, Cf,YZbp),(Xj,Cf,YZbq),(Xj,Cf,YZbr),(Xj,Cf,YZbs),(Xj,Cf,YZbt),(Xj Cf,YZbu),(Xj, Cf,YZbv),(Xj,Cf,YZbw),(Xj,Cf,YZbx),(Xj,Cf,YZby),(Xj,Cf,YZbz),(Xj,Cf,YZca),(Xj, Cf,YZcb),(Xj,Cf,YZcc),(Xj,Cf,YZcd),(Xj,Cf,YZce),(Xj,Cf,YZcf),(Xj,Cf,YZcg),(Xj,Cf, YZch),(Xj,Cf,YZci),(Xj,Cf,YZcj),(Xj,Cf,YZck),(Xj,Cf,YZcl),(Xj,Cf,YZcm),(Xj,Cf,YZcn), (Xj,Cf,YZco),(Xj,Cf,YZcp),(Xj,Cf,YZcq),(Xj,Cf,YZcr),(Xj,Cf,YZcs),(Xj,Cf,YZct), (Xj,Cf,YZcu),(Xj,Cf,YZcv),(Xj,Cf,YZcw),(Xj,Cf;YZcx),(Xj,Cf;YZcy),(Xj,Cf,YZcz), (Xj,Cf,YZda),(Xj,Cf,YZdb),(Xj,Cf,YZdc),(Xj,Cf,YZdd),(Xj,Cf,YZde),(Xj,Cf,YZdf),(Xj, Cf,YZdg),(Xj,Cf,YZdh),(Xj,Cf,YZdi),(Xj,Cf,YZdj),(Xj,Cf,YZdk),(Xj,Cf,YZdl),(Xj, Cf,YZdm),(Xj,Cf,YZdn),(Xj,Cf,YZdo),(Xj,Cf,YZdp),(Xj,Cf,YZdq),(Xj,Cf,YZdr),(Xj, Cf,YZds),(Xj,Cf,Yzdt),(Xj,Cf,YZdu),(Xj,Cf,YZdv),(Xj,Cf,YZdw),(Xj,Cf,YZdx),(Xj,Cf, YZdy),(Xj,Cf,YZdz),(Xj,Cf,YZea),(Xj,Cf,YZeb),(Xj,Cf,YZec),(Xj,Cf,YZed),(Xj,Cf, YZee),(Xj,Cf,YZef),(Xj,Cf,YZeg),(Xj,Cf,YZeh),(Xj,Cf,YZci),(Xj,Cf,YZej),(Xj,Cf,YZek), (Xj,Cf,YZel),(Xj,Cf,YZem),(Xj,Cf,YZen),(Xj,Cf,YZeo),(Xj,Cf,YZep),(Xj,Cf,YZeq), (Xj,Cf,YZer),(Xj,Cf,YZes),(Xj,Cf,YZet),(Xj,Cf,YZeu),(Xj,Cf,YZev),(Xj,Cf,YZew), (Xj,Cf,YZex),(Xj,Cf,YZey),(Xj,Cf,YZez),(Xj,Cf,YZfa),(Xj,Cf,YZfb),(Xj,Cf,YZfc),(Xj, Cf,YZfd),(Xj,Cf,YZfe),(Xj,Cf,YZff),(Xj,Cf,YZfg),(Xj,Cf,YZfh),(Xj,Cf,YZfi),(Xj,Cf, YZfj),(Xj,Cf,YZfk),(Xj,Cf,YZfl),(Xj,Cf,YZfm),(Xj,Cf,YZfn),(Xj,Cf,YZfo),(Xj,Cf,YZfp), (Xj,Cf,YZfq),(Xj,Cf,YZfr),(Xj,Cf,YZfs),(Xj,Cf,YZft),(Xj,Cf,YZfu),(Xj,Cf,YZfv),(Xj, Cf,YZfw),(Xj,Cf,YZfx),(Xj,Cf,YZfy),(Xj,Cf,YZfz),(Xj,Cf,YZga),(Xj,Cf,YZgb),(Xj, Cf,YZgc),(Xj,Cf,YZgd),(Xj,Cf,YZge),(Xj,Cf,YZgf),(Xj,Cf,YZgg),(Xj,Cf,YZgh),(Xj, Cf,YZgi),(Xj,Cf,YZgj),(Xj,Cf,YZgk),(Xj,Cf,YZgl),(Xj,Cf,YZgm),(Xj,Cf,YZgn),(Xj,Cf, YZgo),(Xj,Cf,YZgp),(Xj,Cf,YZgq),(Xj,Cf,YZgr),(Xj,Cf,YZgs),(Xj,Cf,YZgt), (Xk,Ca,YZa),(Xk,Ca,YZb),(Xk,Ca,YZc),(Xk,Ca,YZd),(Xk,Ca,YZe),(Xk,Ca,YZf),(Xk, Ca,YZg),(Xk,Ca,YZh),(Xk,Ca,YZi),(Xk,Ca,YZj),(Xk,Ca,YZk),(Xk,Ca,YZl),(Xk,Ca,YZm), (Xk,Ca,YZn),(Xk,Ca,YZo),(Xk,Ca,YZp),(Xk,Ca,YZq),(Xk,Ca,YZr),(Xk,Ca,YZs), (Xk,Ca,YZt),(Xk,Ca,YZu),(Xk,Ca,YZv),(Xk,Ca,YZw),(Xk,Ca,YZx),(Xk,Ca,YZy),(Xk, Ca,YZz),(Xk,Ca,YZaa),(Xk,Ca,YZab),(Xk,Ca,YZac),(Xk,Ca,YZad),(Xk,Ca,YZae),(Xk, Ca,YZaf),(Xk,Ca,YZag),(Xk,Ca,YZah),(Xk,Ca,YZai),(Xk,Ca,YZaj),(Xk,Ca,YZak), (Xk,Ca,YZal),(Xk,Ca,YZam),(Xk,Ca,YZan),(Xk,Ca,YZao),(Xk,Ca,YZap),(Xk,Ca,YZaq), (Xk,Ca,YZar),(Xk,Ca,YZas),(Xk,Ca,YZat),(Xk,Ca,YZau),(Xk,Ca,YZav),(Xk,Ca, YZaw),(Xk,Ca,YZax),(Xk,Ca,YZay),(Xk,Ca,YZaz),(Xk,Ca,YZba),(Xk,Ca,YZbb),(Xk, Ca,YZbc),(Xk,Ca,YZbd),(Xk,Ca,YZbe),(Xk,Ca,YZbf),(Xk,Ca,YZbg),(Xk,Ca,YZbh),(Xk, Ca,YZbi),(Xk,Ca,YZbj),(Xk,Ca,YZbk),(Xk,Ca,YZbl),(Xk,Ca,YZbm),(Xk,Ca,YZbn), (Xk,Ca,YZbo),(Xk,Ca,YZbp),(Xk,Ca,YZbq),(Xk,Ca,YZbr),(Xk,Ca,YZbs),(Xk,Ca, YZbt),(Xk,Ca,YZbu),(Xk,Ca,YZbv),(Xk,Ca,YZbw),(Xk,Ca,YZbx),(Xk,Ca,YZby),(Xk, Ca,YZbz),(Xk,Ca,YZca),(Xk,Ca,YZcb),(Xk,Ca,YZcc),(Xk,Ca,YZcd),(Xk,Ca,YZce),(Xk, Ca,YZcf),(Xk,Ca,YZcg),(Xk,Ca,YZch),(Xk,Ca,YZci),(Xk,Ca,YZcj),(Xk,Ca,YZck), (Xk,Ca,YZcl),(Xk,Ca,YZcm),(Xk,Ca,YZcn),(Xk,Ca,YZco),(Xk,Ca,YZcp),(Xk,Ca,YZcq), (Xk,Ca,YZcr),(Xk,Ca,YZcs),(Xk,Ca,YZet),(Xk,Ca,YZcu),(Xk,Ca,YZcv),(Xk,Ca, YZcw),(Xk,Ca,YZcx),(Xk,Ca,YZcy),(Xk,Ca,YZcz),(Xk,Ca,YZda),(Xk,Ca,YZdb),(Xk, Ca,YZdc),(Xk,Ca,YZdd),(Xk,Ca,YZde),(Xk,Ca,YZdf),(Xk,Ca,YZdg),(Xk,Ca,YZdh),(Xk, Ca,YZdi),(Xk,Ca,YZdj),(Xk,Ca,YZdk),(Xk,Ca,YZdl),(Xk,Ca,YZdm),(Xk,Ca,YZdn), (Xk,Ca,YZdo),(Xk,Ca,YZdp),(Xk,Ca,YZdq),(Xk,Ca,YZdr),(Xk,Ca,YZds),(Xk,Ca, Yzdt),(Xk,Ca,YZdu),(Xk,Ca,YZdv),(Xk,Ca,YZdw),(Xk,Ca,YZdx),(Xk,Ca,YZdy),(Xk, Ca,YZdz),(Xk,Ca,YZea),(Xk,Ca,YZeb),(Xk,Ca,YZec),(Xk,Ca,YZed),(Xk,Ca,YZee),(Xk, Ca,YZef),(Xk,Ca,YZeg),(Xk,Ca,YZeh),(Xk,Ca,YZei),(Xk,Ca,YZej),(Xk,Ca,YZek), (Xk,Ca,YZel),(Xk,Ca,YZem),(Xk,Ca,YZen),(Xk,Ca,YZeo),(Xk,Ca,YZep),(Xk,Ca,YZeq), (Xk,Ca,YZer),(Xk,Ca,YZes),(Xk,Ca,YZet),(Xk,Ca,YZeu),(Xk,Ca,YZev),(Xk,Ca, YZew),(Xk,Ca,YZex),(Xk,Ca,YZey),(Xk,Ca,YZez),(Xk,Ca,YZfa),(Xk,Ca,YZfb),(Xk, Ca,YZfc),(Xk,Ca,YZfd),(Xk,Ca,YZfe),(Xk,Ca,YZf),(Xk,Ca,YZfg),(Xk,Ca,YZfh),(Xk, Ca,YZfi),(Xk,Ca,YZfj),(Xk,Ca,YZfk),(Xk,Ca,YZfl),(Xk,Ca,YZfm),(Xk,Ca,YZfm),(Xk, Ca,YZfo),(Xk,Ca,YZfp),(Xk,Ca,YZfq),(Xk,Ca,YZfr),(Xk,Ca,YZfs),(Xk,Ca,YZft),(Xk, Ca,YZfu),(Xk,Ca,YZfv),(Xk,Ca,YZfw),(Xk,Ca,YZfx),(Xk,Ca,YZfy),(Xk,Ca,YZfz),(Xk, Ca,YZga),(Xk,Ca,YZgb),(Xk,Ca,YZgc),(Xk,Ca,YZgd),(Xk,Ca,YZge),(Xk,Ca,YZgf), (Xk,Ca,YZgg),(Xk,Ca,YZgh),(Xk,Ca,YZgi),(Xk,Ca,YZgj),(Xk,Ca,YZgk),(Xk,Ca,YZgl), (Xk,Ca,YZgm),(Xk,Ca,YZgn),(Xk,Ca,YZgo),(Xk,Ca,YZgp),(Xk,Ca,YZgq),(Xk, Ca,YZgr),(Xk,Ca,YZgs),(Xk,Ca,YZgt),(Xk,Cb,YZa),(Xk,Cb,YZb),(Xk,Cb,YZc),(Xk, Cb,YZd),(Xk,Cb,YZe),(Xk,Cb,YZf),(Xk,Cb,YZg),(Xk,Cb,YZh),(Xk,Cb,YZi),(Xk,Cb, YZj),(Xk,Cb,YZk),(Xk,Cb,YZl),(Xk,Cb,YZm),(Xk,Cb,YZn),(Xk,Cb,YZo),(Xk,Cb,YZp), (Xk,Cb,YZq),(Xk,Cb,YZr),(Xk,Cb,YZs),(Xk,Cb,YZt),(Xk,Cb,YZu),(Xk,Cb,YZv),(Xk, Cb,YZw),(Xk,Cb,YZx),(Xk,Cb,YZy),(Xk,Cb,YZz),(Xk,Cb,YZaa),(Xk,Cb,YZab),(Xk, Cb,YZac),(Xk,Cb,YZad),(Xk,Cb,YZae),(Xk,Cb,YZaf),(Xk,Cb,YZag),(Xk,Cb,YZah), (Xk,Cb,YZai),(Xk,Cb,YZaj),(Xk,Cb,YZak),(Xk,Cb,YZal),(Xk,Cb,YZam),(Xk,Cb, YZan),(Xk,Cb,YZao),(Xk,Cb,YZap),(Xk,Cb,YZaq),(Xk,Cb,YZar),(Xk,Cb,YZas),(Xk, Cb,YZat),(Xk,Cb,YZau),(Xk,Cb,YZav),(Xk,Cb,YZaw),(Xk,Cb,YZax),(Xk,Cb,YZay), (Xk,Cb,YZaz),(Xk,Cb,YZba),(Xk,Cb,YZbb),(Xk,Cb,YZbc),(Xk,Cb,YZbd),(Xk,Cb,YZbe), (Xk,Cb,YZbf),(Xk,Cb,YZbg),(Xk,Cb,YZbh),(Xk,Cb,YZbi),(Xk,Cb,YZbj),(Xk,Cb, YZbk),(Xk,Cb,YZbl),(Xk,Cb,YZbm),(Xk,Cb,YZbn),(Xk,Cb,YZbo),(Xk,Cb,YZbp),(Xk, Cb,YZbq),(Xk,Cb,YZbr),(Xk,Cb,YZbs),(Xk,Cb,YZbt),(Xk,Cb,YZbu),(Xk,Cb,YZbv), (Xk,Cb,YZbw),(Xk,Cb,YZbx),(Xk,Cb,YZby),(Xk,Cb,YZbz),(Xk,Cb,YZca),(Xk,Cb, YZeb),(Xk,Cb,YZcc),(Xk,Cb,YZcd),(Xk,Cb,YZce),(Xk,Cb,YZcf),(Xk,Cb,YZcg),(Xk, Cb,YZch),(YXk,Cb,YZci),(Xk,Cb,YZcj),(Xk,Cb,YZck),(Xk,Cb,YZcl),(Xk,Cb,YZcm),(Xk, Cb,YZcn),(Xk,Cb,YZco),(Xk,Cb,YZcp),(Xk,Cb,YZcq),(Xk,Cb,YZcr),(Xk,Cb,YZcs), (Xk,Cb,YZct),(Xk,Cb,YZcu),(Xk,Cb,YZcv),(Xk,Cb,YZcw),(Xk,Cb,YZcx),(Xk,Cb, YZcy),(Xk,Cb,YZcz),(Xk,Cb,YZda),(Xk,Cb,YZdb),(Xk,Cb,YZdc),(Xk,Cb,YZdd),(Xk, Cb,YZde),(Xk,Cb,YZdf),(Xk,Cb,YZdg),(Xk,Cb,YZdh),(Xk,Cb,YZdi),(Xk,Cb,YZdj),(Xk, Cb,YZdk),(Xk,Cb,YZdl),(Xk,Cb,YZdm),(Xk,Cb,YZdn),(Xk,Cb,YZdo),(Xk,Cb,YZdp), (Xk,Cb,YZdq),(Xk,Cb,YZdr);(Xk,Cb,YZds),(Xk,Cb,Yzdt),(Xk,Cb,YZdu),(Xk,Cb, YZdv),(Xk,Cb,YZdw),(Xk,Cb,YZdx),(Xk,Cb,YZdy),(Xk,Cb,YZdz),(Xk,Cb,YZea),(Xk, Cb,YZeb),(Xk,Cb,YZec),(Xk,Cb,YZed),(Xk,Cb,YZee),(Xk,Cb,YZef),(Xk,Cb,YZeg), (Xk,Cb,YZeh),(Xk,Cb,YZei),(Xk,Cb,YZej),(Xk,Cb,YZek),(Xk,Cb,YZel),(Xk,Cb,YZem), (Xk,Cb,YZen),(Xk,Cb,YZeo),(Xk,Cb,YZep),(Xk,Cb,YZeq),(Xk,Cb,YZer),(Xk,Cb, YZes),(Xk,Cb,YZet),(Xk,Cb,YZeu),(Xk,Cb,YZev),(Xk,Cb,YZew),(Xk,Cb,YZex),(Xk, Cb,YZey),(Xk,Cb,YZez),(Xk,Cb,YZfa),(Xk,Cb,YZfb),(Xk,Cb,YZfc),(Xk,Cb,YZfd),(Xk, Cb,YZfe),(Xk,Cb,YZff),(Xk,Cb,YZfg),(Xk,Cb,YZfh),(Xk,Cb,YZfi),(Xk,Cb,YZfj), (Xk,Cb,YZfk),(Xk,Cb,YZfl),(Xk,Cb,YZfm),(Xk,Cb,YZfn),(Xk,Cb,YZfo),(Xk,Cb,YZfp), (Xk,Cb,YZfq),(Xk,Cb,YZfr),(Xk,Cb,YZfs),(Xk,Cb,YZft),(Xk,Cb,YZfu),(Xk,Cb,YZfv), (Xk,Cb,YZfw),(Xk,Cb,YZfx),(Xk,Cb,YZfy),(Xk,Cb,YZfz),(Xk,Cb,YZga),(Xk,Cb, YZgb),(Xk,Cb,YZgc),(Xk,Cb,YZgd),(Xk,Cb,YZge),(Xk,Cb,YZgf),(Xk,Cb,YZgg),(Xk, Cb,YZgh),(Xk,Cb,YZgi),(Xk,Cb,YZgj),(Xk,Cb,YZgk),(Xk,Cb,YZgl),(Xk,Cb,YZgm), (Xk,Cb,YZgn),(Xk,Cb,YZgo),(Xk,Cb,YZgp),(Xk,Cb,YZgq),(Xk,Cb,YZgr),(Xk,Cb,YZgs), (Xk,Cb,YZgt),(Xk,Cc,YZa),(Xk,Cc,YZb),(Xk,Cc,YZc),(Xk,Cc,YZd),(Xk,Cc,YZe), (Xk,Cc,YZf),(Xk,Cc,YZg),(Xk,Cc,YZh),(Xk,Cc,YZi),(Xk,Cc,YZj),(Xk,Cc,YZk),(Xk, Cc,YZl),(Xk,Cc,YZm),(Xk,Cc,YZn),(Xk,Cc,YZo),(Xk,Cc,YZp),(Xk,Cc,YZq),(Xk,Cc, YZr),(Xk,Cc,YZs),(Xk,Cc,YZt),(Xk,Cc,YZu),(Xk,Cc,YZv),(Xk,Cc,YZw),(Xk,Cc,YZx), (Xk,Cc,YZy),(Xk,Cc,YZz),(Xk,Cc,YZaa),(Xk,Cc,YZab),(Xk,Cc,YZac),(Xk,Cc,YZad), (Xk,Cc,YZae),(Xk,Cc,YZaf),(Xk,Cc,YZag),(Xk,Cc,YZah),(Xk,Cc,YZai),(Xk,Cc, YZaj),(Xk,Cc,YZak),(Xk,Cc,YZal),(Xk,Cc,YZam),(Xk,Cc,YZan),(Xk,Cc,YZao),(Xk, Cc,YZap),(Xk,Cc,YZaq),(Xk,Cc,YZar),(Xk,Cc,YZas),(Xk,Cc,YZat),(Xk,Cc,YZau),(Xk, Cc,YZav),(Xk,Cc,YZaw),(Xk,Cc,YZax),(Xk,Cc,YZay),(Xk,Cc,YZaz),(Xk,Cc,YZba), (Xk,Cc,YZbb),(Xk,Cc,YZbc),(Xk,Cc,YZbd),(Xk,Cc,YZbe),(Xk,Cc,YZbf),(Xk,Cc,YZbg), (Xk,Cc,YZbh),(Xk,Cc,YZbi),(Xk,Cc,YZbj),(Xk,Cc,YZbk),(Xk,Cc,YZbl),(Xk,Cc, YZbm),(Xk,Cc,YZbn),(Xk,Cc,YZbo),(Xk,Cc,YZbp),(Xk,Cc,YZbq),(Xk,Cc,YZbr),(Xk, Cc,YZbs),(Xk,Cc,YZbt),(Xk,Cc,YZbu),(Xk,Cc,YZbv),(Mc,Cc,YZbw),(X(k,Cc,YZbx), (Xk,Cc,YZby),(Xk,Cc,YZbz),(Xk,Cc,YZca),(Xk,Cc,YZcb),(Xk,Cc,YZcc),(Xk,Cc,YZcd), (Xk,Cc,YZce),(Xk,Cc,YZcf),(Xk,Cc,YZcg),(Xk,Cc,YZch),(Xk,Cc,YZci),(Xk,Ce, YZcj),(Xk,Cc,YZck),(Xk,Cc,YZcl),(Xk,Cc,YZcm),(Xk,Cc,YZcn),(Xk,Cc,YZco),(Xk, Cc,YZcp),(Xk,Cc,YZcq),(Xk,Cc,YZcr),(Xk,Cc,YZes),(Xk,Cc,YZet),(Xk,Cc,YZcu),(Xk, Cc,YZcv),(Xk,Cc,YZcw),(Xk,Cc,YZcx),(Xk,Cc,YZcy),(Xk,Cc,YZcz),(Xk,Cc,YZda), (Xk,Cc,YZdb),(Xk,Cc,YZdc),(Xk,Cc,YZdd),(Xk,Cc,YZde),(Xk,Cc,YZdf),(Xk,Cc,YZdg), (Xk,Cc,YZdh),(Xk,Cc,YZdi),(Xk,Cc,YZdj),(Xk,Cc,YZdk),(Xk,Cc,YZdl),(Xk,Cc, YZdm),(Xk,Cc,YZdn),(Xk,Cc,YZdo),(Xk,Cc,YZdp),(Xk,Cc,YZdq),(Xk,Cc,YZdr),(Xk, Cc,YZds),(Xk,Cc,Yzdt),(Xk,Cc,YZdu),(Xk,Cc,YZdv),(Xk,Cc,YZdw),(Xk,Cc,YZdx), (Xk,Cc,YZdy),(Xk,Cc,YZdz),(Xk,Cc,YZea),(Xk,Cc,YZeb),(Xk,Cc,YZec),(Xk,Cc,YZed), (Xk,Cc,YZee),(Xk,Cc,YZef),(Xk,Cc,YZeg),(Xk,Cc,YZeh),(Xk,Cc,YZei),(Xk,Cc, YZej),(Xk,Cc,YZek),(Xk,Cc,YZel),(Xk,Cc,YZem),(Xk,Cc,YZen),(Xk,Cc,YZeo),(Xk, Cc,YZep),(Xkc,Cc,YZeq),(Xk,Cc,YZer),(Xk,Cc,YZes),(Xk,Cc,YZet),(Xk,Cc,YZeu),(Xk, Cc,YZev),(Xk,Cc,YZew),(Xk,Cc,YZex),(Xk,Cc,YZey),(Xk,Cc,YZez),(Xk,Cc,YZfa), (Xk,Cc,YZfb),(Xk,Cc,YZfc),(Xk,Cc,YZfd),(Xk,Cc,YZfe),(Xk,Cc,YZff),(Xk,Cc,YZfg), (Xk,Cc,YZfh),(Xk,Cc,YZfi),(Xk,Cc,YZfj),(Xk,Cc,YZfk),(Xk,Cc,YZfl),(Xk,Cc,YZfm), (Xk,Cc,YZfn),(Xk,Cc,YZfo),(Xk,Cc,YZfp),(Xk,Cc,YZfq),(Xk,Cc,YZfr),(Xk,Cc,YZfs), (Xk,Cc,YZft),(Xk,Cc,YZfu),(Xk,Cc,YZfv),(Xk,Cc,YZfwv),(Xk,Cc,YZfx),(Xk,Cc, YZfy),(Xk,Cc,YZfz),(Xk,Cc,YZga),(Xk,Cc,YZgb),(Xk,Cc,YZgc),(Xk,Cc,YZgd),(Xk, Cc,YZge),(Xk,Cc,YZgf),(Xk,Cc,YZgg),(Xk,Cc,YZgh),(Xk,Cc,YZgi),(Xk,Cc,YZg),(Xk, Cc,YZgk),(Xk,Cc,YZgl),(Xk,Cc,YZgm),(Xk,Cc,YZgn),(Xk,Cc,YZgo),(Xk,Cc,YZgp), (Xk,Cc,YZgq),(Xk,Cc,YZgr),(Xk,Cc,YZgs),(Xk,Cc,YZgt),(Xk,Cd,YZa),(Xk,Cd,YZb), (Xk,Cd,YZc),(Xk,Cd,YZd),(Xk,Cd,YZe),(Xk,Cd,YZf),(Xk,Cd,YZg),(Xk,Cd,YZh), (Xk,Cd,YZi),(Xk,Cd,YZj),(Xk,Cd,YZk),(Xk,Cd,YZl),(Xk,Cd,YZm),(Xk,Cd,YZn),(Xk, Cd,YZo),(Xk,Cd,YZp),(Xk,Cd,YZq),(Xk,Cd,YZr),(Xk,Cd,YZs),(Xk,Cd,YZt),(Xk,Cd, YZu),(Xk,Cd,YZv),(Xk,Cd,YZw),(Xk,Cd,YZx),(Xk,Cd,YZy),(Xk,Cd,YZz),(Xk,Cd, YZaa),(Xk,Cd,YZab),(Xk,Cd,YZac),(Xk,Cd,YZad),(Xk,Cd,YZae),(Xk,Cd,YZaf),(Xk, Cd,YZag),(Xk,Cd,YZah),(Xk,Cd,YZai),(Xk,Cd,YZaj),(Xk,Cd,YZak),(Xk,Cd,YZal),(Xk, Cd,YZam),(Xk,Cd,YZan),(Xk,Cd,YZao),(Xk,Cd,YZap),(Xk,Cd,YZaq),(Xk,Cd,YZar), (Xk,Cd,YZas),(Xk,Cd,YZat),(Xk,Cd,YZau),(Xk,Cd,YZav),(Xk,Cd,YZaw),(Xk,Cd, YZax),(Xk,Cd,YZay),(Xk,Cd,YZaz),(Xk,Cd,YZba),(Xk,Cd,YZbb),(Xk,Cd,YZbc),(Xk, Cd,YZbd),(Xk,Cd,YZbe),(Xk,Cd,YZbf),(Xk,Cd,YZbg),(Xk,Cd,YZbh),(Xk,Cd,YZbi), (Xk,Cd,YZbj),(Xk,Cd,YZbk),(X,Cd,YZbl),(Xk,Cd,YZbm),(Xk,Cd,YZbn),(Xk,Cd,YZbo), (Xk,Cd,YZbp),(Xk,Cd,YZbq),(Xk,Cd,YZbr),(Xk,Cd,YZbs),(Xk,Cd,YZbt),(Xk,Cd, YZbu),(Xk,Cd,YZbv),(Xk,Cd,YZbw),(Xk,Cd,YZbx),(Xk,Cd,YZby),(Xk,Cd,YZbz),(Xk, Cd,YZca),(Xk,Cd,YZcb),(Xk,Cd,YZec),(Xk,Cd,YZcd),(Xk,Cd,YZce),(Xk,Cd,YZcf), (Xk,Cd,YZcg),(Xk,Cd,YZch),(Xk,Cd,YZci),(Xk,Cd,YZcj),(Xk,Cd,YZck),(Xk,Cd,YZcl), (YXk,Cd,YZcm),(Xk,Cd,YZcn),(Xk,Cd,YZco),(Xk,Cd,YZcp),(Xk,Cd,YZcq),(Xk, Cd,YZcr),(Xk,Cd,YZcs),(Xk,Cd,YZct),(Xk,Cd,YZcu),(Xk,Cd,YZcv),(Xk,Cd,YZcw),(Xk, Cd,YZcx),(Xk,Cd,YZcy),(Xk,Cd,YZcz),(Xk,Cd,YZda),(Xk,Cd,YZdb),(Xk,Cd,YZdc), (Xk,Cd,YZdd),(Xk,Cd,YZde),(Xk,Cd,YZdf),(Xk,Cd,YZdg),(Xk,Cd,YZdh),(Xk,Cd, YZdi),(Xk,Cd,YZdj),(Xk,Cd,YZdk),(Xk,Cd,YZdl),(Xk,Cd,YZdm),(Xk,Cd,YZdn),(Xk, Cd,YZdo),(Xk,Cd,YZdp),(Xk,Cd,YZdq),(Xk,Cd,YZdr),(Xk,Cd,YZds),(Xk,Cd,Yzdt), (Xk,Cd,YZdu),(Xk,Cd,YZdv),(Xk,Cd,YZdw),(Xk,Cd,YZdx),(Xk,Cd,YZdy),(Xk,Cd, YZdz),(Xk,Cd,YZea),(Xk,Cd,YZeb),(Xk,Cd,YZec),(Xk,Cd,YZed),(Xk,Cd,YZee),(Xk, Cd,YZef),(Xk,Cd,YZeg),(Xk,Cd,YZeh),(Xk,Cd,YZei),(Xk,Cd,YZej),(Xk,Cd,YZek),(Xk, Cd,YZel),(Xk,Cd,YZem),(Xk,Cd,YZen),(Xk,Cd,YZeo),(Xk,Cd,YZep),(Xk,Cd,YZeq), (Xk,Cd,YZer),(Xk,Cd,YZes),(Xk,Cd,YZet),(Xk,Cd,YZeu),(Xk,Cd,YZev),(Xk,Cd, YZew),(Xk,Cd,YZex),(Xk,Cd,YZey),(Xk,Cd,YZez),(Xk,Cd,YZfa),(Xk,Cd,YZfb),(Xk, Cd,YZfc),(Xk,Cd,YZfd),(Xk,Cd,YZfe),(Xk,Cd,YZff),(Xk,Cd,YZfg),(Xk,Cd,YZfh),(Xk, Cd,YZfi),(Xk,Cd,YZfj),(Xk,Cd,YZfk),(Xk,Cd,YZfl),(Xk,Cd,YZfm),(Xk,Cd,YZfn),(Xk, Cd,YZfo),(Xk,Cd,YZfp),(Xk,Cd,YZfq),(Xk,Cd,YZfr),(Xk,Cd,YZfs),(Xk,Cd,YZft), (Xk,Cd,YZfu),(Xk,Cd,YZfv),(Xk,Cd,YZfw),(Xk,Cd,YZfx),(Xk,Cd,YZfy),(Xk,Cd,YZfz), (Xk,Cd,YZga),(Xk,Cd,YZgb),(Xk,Cd,YZgc),(Xk,Cd,YZgd),(Xk,Cd,YZge),(YXk,Cd, YZgf),(Yk,Cd,YZgg),(Xk,Cd,YZgh),(Xk,Cd,YZgi),(Xk,Cd,YZgj),(Xk,Cd,YZgk),(Xk, Cd,YZgl),(Xk,Cd,YZgm),(Xk,Cd,YZgn),(Xk,Cd,YZgo),(Xk,Cd,YZgp),(Xk,Cd,YZgq), (Xk,Cd,YZgr),(Xk,Cd,YZgs),(Xk,Cd,YZgt),(Xk,Ce,YZa),(Xk,Ce,YZb),(Xk,Ce,YZc), (Xk,Ce,YZd),(Xk,Ce,YZe),(Xk,Ce,YZf),(Xk,Ce,YZg),(Xk,Ce,YZh),(Xk,Ce,YZi),(Xk, Ce,YZj),(Xk,Ce,YZk),(Xk,Ce,YZl),(Xk,Ce,YZm),(Xk,Ce,YZn),(Xk,Ce,YZo),(Xk,Ce, YZp),(Xk,Ce,YZq),(Xk,Ce,YZr),(Xk,Ce,YZs),(Xk,Ce,YZt),(Xk,Ce,YZu),(Xk,Ce,YZv), (Xk,Ce,YZw),(Xk,Ce,YZx),(Xk,Ce,YZy),(Xk,Ce,YZz),(Xk,Ce,YZaa),(Xk,Ce,YZab), (Xk,Ce,YZac),(Xk,Ce,YZad),(Xk,Ce,YZae),(Xk,Ce,YZaf),(Xk,Ce,YZag),(Xk,Ce,YZah), (Xk,Ce,YZai),(Xk,Ce,YZaj),(Xk,Ce,YZak),(Xk,Ce,YZal),(Xk,Ce,YZam),(Xk,Ce, YZan),(Xk,Ce,YZao),(Xk,Ce,YZap),(Xk,Ce,YZaq),(Xk,Ce,YZar),(Xk,Ce,YZas),(Xk, Ce,YZat),(Xk,Ce,YZau),(Xk,Ce,YZav),(Xk,Ce,YZaw),(Xk,Ce,YZax),(Xk,Ce,YZay),(Xk, Ce,YZaz),(Xk,Ce,YZba),(Xk,Ce,YZbb),(Xk,Ce,YZbc),(Xk,Ce,YZbd),(Xk,Ce,YZbe), (Xk,Ce,YZbf),(Xk,Ce,YZbg),(Xk,Ce,YZbh),(Xk,Ce,YZbi),(Xk,Ce,YZbj),(Xk,Ce,YZbk), (Xk,Ce,YZbl),(Xk,Ce,YZbm),(Xk,Ce,YZbn),(Xk,Ce,YZbo),(Xk,Ce,YZbp),(Xk, Ce,YZbq),(Xk,Ce,YZbr),(Xk,Ce,YZbs),(Xk,Ce,YZbt),(xk,Ce,YZbu),(Xk,Ce,YZbv),(Xk, Ce,YZbw),(Xk,Ce,YZbx),(Xk,Ce,YZby),(Xk,Ce,YZbz),(Xk,Ce,YZca),(Xk,Ce,YZcb), (Xk,Ce,YZcc),(Xk,Ce,YZcd),(Xk,Ce,YZce),(Xk,Ce,YZcf),(Xk,Ce,YZcg),(Xk,Ce, YZch),(Xk,Ce,YZci),(Xk,Ce,YZcj),(Xk,Ce,YZck),(Xk,Ce,YZcl),(Xk,Ce,YZcm),(Xk, Ce,YZcn),(Xk,Ce,YZco),(Xk,Ce,YZcp),(Xk,Ce,YZcq),(Xk,Ce,YZcr),(Xk,Ce,YZcs),(Xk, Ce,YZct),(Xk,Ce,YZcu),(Xk,Ce,YZcv),(Xk,Ce,YZcw),(Xk,Ce,YZcx),(Xk,Ce,YZcy), (Xk,Ce,YZcz),(Xk,Ce,YZda),(Xk,Ce,YZdb),(Xk,Ce,YZdc),(Xk,Ce,YZdd),(Xk,Ce, YZde),(Xk,Ce,YZdf),(Xk,Ce,YZdg),(Xk,Ce,YZdh),(Xk,Ce,YZdi),(Xk,Ce,YZdj),(Xk, Ce,YZdk),(Xk,Ce,YZdl),(Xk,Ce,YZdm),(Xk,Ce,YZdn),(Xk,Ce,YZdo),(Xk,Ce,YZdp), (Xk,Ce,YZdq),(Xk,Ce,YZdr),(Xk,Ce,YZds),(Xk,Ce,Yzdt),(Xk,Ce,YZdu),(Xk,Ce,YZdv), (Xk,Ce,YZdw),(Xk,Ce,YZdx),(Xk,Ce,YZdy),(Xk,Ce,YZdz),(Xk,Ce,YZea),(Xk,Ce, YZeb),(Xk,Ce,YZec),(Xk,Ce,YZed),(Xk,Ce,YZee),(Xk,Ce,YZef),(Xk,Ce,YZeg),(Xk, Ce,YZeh),(Xk,Ce,YZei),(Xk,Ce,YZej),(YXk,Ce,YZek),(Xk,Ce,YZel),(Xk,Ce,YZem),(Xk, Ce,YZen),(Xk,Ce,YZeo),(Xk,Ce,YZep),(Xk,Ce,YZeq),(Xk,Ce,YZer),(Xk,Ce,YZes), (Xk,Ce,YZet),(Xk,Ce,YZeu),(Xk,Ce,YZev),(Xk,Ce,YZew),(Xk,Ce,YZex),(Xk,Ce,YZey), (Xk,Ce,YZez),(Xk,Ce,YZfa),(Xk,Ce,YZfb),(Xk,Ce,YZfc),(Xk,Ce,YZfd),(Xk,Ce, YZfe),(Xk,Ce,YZff),(Xk,Ce,YZfg),(Xk,Ce,YZfh),(Xk,Ce,YZfi),(Xk,Ce,YZfj),(Xk,Ce, YZfk),(Xk,Ce,YZfl),(Xk,Ce,YZfm),(Xk,Ce,YZfn),(Xk,Ce,YZfo),(Xk,Ce,YZfp),(Xk,Ce, YZfq),(Xk,Ce,YZfr),(Xk,Ce,YZfs),(Xk,Ce,YZft),(Xk,Ce,YZfu),(Xk,Ce,YZfv),(Xk,Ce, YZfw),(Xk,Ce,YZfx),(Xk,Ce,YZfy),(Xk,Ce,YZfz),(Xk,Ce,YZga),(Xk,Ce,YZgb),(Xk, Ce,YZgc),(Xk,Ce,YZgd),(Xk,Ce,YZge),(Xk,Ce,YZgf),(Xk,Ce,YZgg),(Xk,Ce,YZgh), (Xk,Ce,YZgi),(Xk,Ce,YZgj),(Xk,Ce,YZgk),(Xk,Ce,YZgl),(Xk,Ce,YZgm),(Xk,Ce,YZgn), (Xk,Ce,YZgo),(Xk,Ce,YZgp),(Xk,Ce,YZgq),(Xk,Ce,YZgr),(Xk,Ce,YZgs),(Xk,Ce, YZgt),(Xk,Cf,YZa),(Xk,Cf,YZb),(Xk,Cf,YZc),(Xk,Cf,YZd),(Xk,Cf,YZe),(Xk,Cf,YZf), (Xk,Cf,YZg),(Xk,Cf,YZh),(Xk,Cf,YZi),(Xk,Cf,YZj),(Xk,Cf,YZk),(Xk,Cf,YZl),(Xk,Cf, YZm),(Xk,Cf,YZn),(Xk,Cf,YZo),(Xk,Cf,YZp),(Xk,Cf,YZq),(Xk,Cf,YZr),(Xk,Cf,YZs), (Xk,Cf,YZt),(Xk,Cf,YZu),(Xk,Cf,YZv),(Xk,Cf,YZw),(Xk,Cf,YZx),(Xk,Cf,YZy),(Xk, Cf,YZz),(Xk,Cf,YZaa),(Xk,Cf,YZab),(Xk,Cf,YZac),(Xk,Cf,YZad),(Xk,Cf,YZae),(Xk, Cf,YZaf),(Xk,Cf,YZag),(Xk,Cf,YZah),(Xk,Cf,YZai),(Xk,Cf,YZaj),(Xk,Cf,YZak),(Xk, Cf,YZal),(Xk,Cf,YZam),(Xk,Cf,YZan),(Xk,Cf,YZao),(Xk,Cf,YZap),(Xk,Cf,YZaq),(Xk, Cf,YZar),(Xk,Cf,YZas),(Xk,Cf,YZat),(Xk,Cf,YZau),(Xk,Cf,YZav),(Xk,Cf,YZaw), (Xk,Cf,YZax),(Xk,Cf,YZay),(Xk,Cf,YZaz),(Xk,Cf,YZba),(Xk,Cf,YZbb),(Xk,Cf,YZbc), (Xk,Cf,YZbd),(Xk,Cf,YZbe),(Xk,Cf,YZbf),(Xk,Cf,YZbg),(Xk,Cf,YZbh),(Xk,Cf,YZbi), (Xk,Cf,YZbj),(Xk,Cf,YZbk),(YXk,Cf,YZbl),(Xk,Cf,YZbm),(Xk,Cf,YZbn),(Xk,Cf,YZbo), (Xk,Cf,YZbp),(Xk,Cf,YZbq),(Xk,Cf,YZbr),(Xk,Cf,YZbs),(Xk,Cf,YZbt),(Xk,Cf, YZbu),(Xk,Cf,YZbv),(Xk,Cf,YZbw),(Xk,Cf,YZbx),(Xk,Cf,YZby),(Xk,Cf,YZbz),(Xk, Cf,YZca),(Xk,Cf,YZcb),(Xk,Cf,YZcc),(Xk,Cf,YZcd),(Xk,Cf,YZce),(Xk,Cf,YZcf),(Xk, Cf,YZcg),(Xk,Cf,YZch),(Xk,Cf,YZci),(Xk,Cf,YZcj),(Xk,Cf,YZck),(Xk,Cf,YZcl),(Xk, Cf,YZcm),(Xk,Cf,YZcn),(Xk,Cf,YZco),(Xk,Cf,YZcp),(Xk,Cf,YZcq),(Xk,Cf,YZcr),(Xk, Cf,YZcs),(Xk,Cf,YZct),(Xk,Cf,YZcu),(Xk,Cf,YZcv),(Xk,Cf,YZcw),(Xk,Cf,YZcx), (Xk,Cf,YZcy),(Xk,Cf,YZcz),(Xk,Cf,YZda),(Xk,Cf,YZdb),(Xk,Cf,YZdc),(Xk,Cf,YZdd), (Xk,Cf,YZde),(Xk,Cf,YZdf),(Xk,Cf,YZdg),(Xk,Cf,YZdh),(Xk,Cf,YZdi),(Xk,Cf,YZdj), (Xk,Cf,YZdk),(Xk,Cf,YZdl),(Xk,Cf,YZdm),(Xk,Cf,YZdn),(Xk,Cf,YZdo),(Xk,Cf, YZdp),(Xk,Cf,YZdq),(Xk,Cf,YZdr),(Xk,Cf,YZds),(Xk,Cf,Yzdt),(Xk,Cf,YZdu),(Xk,Cf, YZdv),(Xk,Cf,YZdw),(Xk,Cf,YZdx),(Xk,Cf,YZdy),(Xk,Cf,YZdz),(Xk,Cf,YZea),(Xk, Cf,YZeb),(Xk,Cf,YZec),(Xk,Cf,YZed),(Xk,Cf,YZee),(Xk,Cf,YZef),(Xk,Cf,YZeg),(Xk, Cf,YZeh),(Xk,Cf,YZei),(Xk,Cf,YZej),(Xk,Cf,YZek),(Xk,Cf,YZel),(Xk,Cf,YZem),(Xk, Cf,YZen),(Xk,Cf,YZeo),(Xk,Cf,YZep),(Xk,Cf,YZeq),(Xk,Cf,YZer),(Xk,Cf,YZes),(Xk, Cf,YZet),(Xk,Cf,YZeu),(Xk,Cf,YZev),(Xk,Cf,YZew),(Xk,Cf,YZex),(Xk,Cf,YZey), (Xk,Cf,YZez),(Xk,Cf,YZfa),(Xk,Cf,YZfb),(Xk,Cf,YZfc),(Xk,Cf,YZfd),(Xk,Cf,YZfe), (Xk,Cf,YZff),(Xk,Cf,YZfg),(Xk,Cf,YZfh),(Xk,Cf,YZfi),(Xk,Cf,YZfj),(Xk,Cf,YZfk),(Xk, Cf,YZfl),(Xk,Cf,YZfm),(Xk,Cf,YZfn),(Xk,Cf,YZfo),(Xk,Cf,YZfp),(Xk,Cf,YZfq),(Xk, Cf,YZfr),(Xk,Cf,YZfs),(Xk,Cf,YZft),(Xk,Cf,YZfu),(Xk,Cf,YZfv),(Xk,Cf,YZfw),(Xk, Cf,YZfx),(Xk,Cf,YZfy),(Xk,Cf,YZfz),(Xk,Cf,YZga),(Xk,Cf,YZgb),(Xk,Cf,YZgc), (Xk,Cf,YZgd),(Xk,Cf,YZge),(Xk,Cf,YZgf),(Xk,Cf,YZgg),(Xk,Cf,YZgh),(Xk,Cf,YZgi), (Xk,Cf,YZg),(Xk,Cf,YZgk),(Xk,Cf,YZgl),(Xk,Cf,YZgm),(Xk,Cf,YZgn),(Xk,Cf,YZgo), (Xk,Cf,YZgp),(Xk,Cf,YZgq),(Xk,Cf,YZgr),(Xk,Cf,YZgs),(Xk,Cf,YZgt), (Xl,Ca,YZa),(Xl,Ca,YZb),(Xl,Ca,YZc),(Xl,Ca,YZd),(Xl,Ca,YZe),(Xl,Ca,YZf),(Xl,Ca, YZg),(Xl,Ca,YZh),(Xl,Ca,YZi),(Xl,Ca,YZj),(Xl,Ca,YZk),(Xl,Ca,YZl),(Xl,Ca,YZm),(Xl, Ca,YZn),(Xl,Ca,YZo),(Xl,Ca,YZp),(Xl,Ca,YZq),(Xl,Ca,YZr),(Xl,Ca,YZs),(Xl,Ca, YZt),(Xl,Ca,YZu),(Xl,Ca,YZv),(Xl,Ca,YZw),(Xl,Ca,YZx),(Xl,Ca,YZy),(Xl,Ca,YZz),(Xl, Ca,YZaa),(Xl,Ca,YZab),(Xl,Ca,YZac),(Xl,Ca,YZad),(Xl,Ca,YZae),(Xl,Ca,YZaf),(Xl, Ca,YZag),(Xl,Ca,YZah),(Xl,(Ca,YZai),(Xl,Ca,YZaj),(Xl,Ca,YZak),(Xl,Ca,YZal),(Xl, Ca,YZam),(Xl,Ca,YZan),(Xl,Ca,YZao),(Xl,Ca,YZap),(Xl,Ca,YZaq),(Xl,Ca,YZar),(Xl, Ca,YZas),(Xl,Ca,YZat),(Xl,Ca,YZau),(Xl,Ca,YZav),(Xl,Ca,YZaw),(Xl,Ca,YZax),(Xl, Ca,YZay),(Xl,Ca,YZaz),(Xl,Ca,YZba),(Xl,Ca,YZbb),(Xl,Ca,YZbc),(Xl,Ca,YZbd),(Xl, Ca,YZbe),(Xl,Ca,YZbf),(Xl,Ca,YZbg),(Xl,Ca,YZbh),(Xl,Ca,YZbi),(Xl,Ca,YZbj),(Xl, Ca,YZbk),(Xl,Ca,YZbl),(Xl,Ca,YZbm),(Xl,Ca,YZbn),(Xl,Ca,YZbo),(Xl,Ca,YZbp),(Xl, Ca,YZbq),(Xl,Ca,YZbr),(Xl,Ca,YZbs),(Xl,Ca,YZbt),(Xl,Ca,YZbu),(Xl,Ca,YZbv),(Xl, Ca,YZbw),(Xl,Ca,YZbx),(Xl,Ca,YZby),(Xl,Ca,YZbz),(Xl,Ca,YZca),(Xl,Ca,YZcb),(Xl, Ca,YZcc),(Xl,Ca,YZcd),(Xl,Ca,YZce),(Xl,Ca,YZcf),(Xl,Ca,YZcg),(Xl,Ca,YZch),(Xl, Ca,YZci),(Xl,Ca,YZcj),(Xl,Ca,YZck),(Xl,Ca,YZcl),(Xl,Ca,YZcm),(Xl,Ca,YZcn),(Xl, Ca,YZco),(Xl,Ca,YZcp),(Xl,Ca,YZcq),(Xl,Ca,YZcr),(Xl,Ca,YZcs),(Xl,Ca,YZct),(Xl,Ca, YZcu),(Xl,Ca,YZcv),(Xl,Ca,YZcw),(Xl,Ca,YZcx),(Xl,Ca,YZcy),(Xl,Ca,YZcz),(Xl,Ca, YZda),(Xl,Ca,YZdb),(Xl,Ca,YZdc),(Xl,Ca,YZdd),(Xl,Ca,YZde),(Xl,Ca,YZdf),(Xl,Ca, YZdg),(Xl,Ca,YZdh),(Xl,Ca,YZdi),(Xl,Ca,YZdj),(Xl,Ca,YZdk),(Xl,Ca,YZdl),(Xl,Ca, YZdm),(Xl,Ca,YZdn),(Xl,Ca,YZdo),(Xl,Ca,YZdp),(Xl,Ca,YZdq),(Xl,Ca,YZdr),(Xl,Ca, YZds),(Xl,Ca,Yzdt),(Xl,Ca,YZdu),(Xl,Ca,YZdv),(Xl,Ca,YZdw),(Xl,Ca,YZdx),(Xl,Ca, YZdy),(Xl,Ca,YZdz),(Xl,Ca,YZea),(Xl,Ca,YZeb),(Xl,Ca,YZec),(Xl,Ca,YZed),(Xl,Ca, YZee),(Xl,Ca,YZef),(Xl,Ca,YZeg),(Xl,Ca,YZeh),(Xl,Ca,YZei),(Xl,Ca,YZej),(Xl,Ca, YZek),(Xl,Ca,YZel),(Xl,Ca,YZem),(Xl,Ca,YZen),(Xl,Ca,YZeo),(Xl,Ca,YZep),(Xl,Ca, YZeq),(Xl,Ca,YZer),(Xl,Ca,YZes),(Xl,Ca,YZet),(Xl,Ca,YZeu),(Xl,Ca,YZev),(Xl,Ca, YZew),(Xl,Ca,YZex),(Xl,Ca,YZey),(Xl,Ca,YZez),(Xl,Ca,YZfa),(Xl,Ca,YZfb),(Xl,Ca, YZfc),(Xl,Ca,YZfd),(Xl,Ca,YZfe),(Xl,Ca,YZff),(Xl,Ca,YZfg),(Xl,Ca,YZfh),(Xl,Ca,YZfi), (Xl,Ca,YZfj),(Xl,Ca,YZfk),(Xl,Ca,YZfl),(Xl,Ca,YZfm),(Xl,Ca,YZfm),(Xl,Ca,YZfo), (Xl,Ca,YZfp),(Xl,Ca,YZfq),(Xl,Ca,YZfr),(Xl,Ca,Yfs),(Xl,Ca,YZft),(Xl,Ca,YZfu), (Xl,Ca,YZfv),(Xl,Ca,YZfw),(Xl,Ca,YZfx)),(Xl,Ca,YZfy),(Xl,Ca,YZfz),(Xl,Ca,YZga),(Xl, Ca,YZgb),(Xl,Ca,YZgc),(Xl,Ca,YZgd),(Xl,Ca,YZge),(Xl,Ca,YZgf),(Xl,Ca,YZgg),(Xl, Ca,YZgh),(Xl,Ca,YZgi),(Xl,Ca,YZgj),(Xl,Ca,YZgk),(Xl,Ca,YZgl),(Xl,Ca,YZgm),(Xl, Ca,YZgn),(Xl,Ca,YZgo),(Xl,Ca,YZgp),(Xl,Ca,YZgq),(Xl,Ca,YZgr),(Xl,Ca,YZgs),(Xl, Ca,YZgt),(Xl,Cb,YZa),(Xl,Cb,YZb),(Xl,Cb,YZc),(Xl,Cb,YZd),(Xl,Cb,YZe),(Xl,Cb, YZf),(Xl,Cb,YZg),(Xl,Cb,YZh),(Xl,Cb,YZi),(Xl,Cb,YZj),(Xl,Cb,YZk),(Xl,Cb,YZl),(Xl, Cb,YZm),(Xl,Cb,YZn),(Xl,Cb,YZo),(Xl,Cb,YZp),(Xl,Cb,YZq),(Xl,Cb,YZr),(Xl,Cb, YZs),(Xl,Cb,YZt),(Xl,Cb,YZu),(Xl,Cb,YZv),(Xl,Cb,YZw),(Xl,Cb,YZx),(Xl,Cb,Yzy), (Xl,Cb,YZz),(Xl,Cb,YZaa),(Xl,Cb,YZab),(Xl,Cb,YZac),(Xl,Cb,YZad),(Xl,Cb,YZae), (Xl,Cb,YZaf),(Xl,Cb,YZag),(Xl,Cb,YZah),(Xl,Cb,YZai),(Xl,Cb,YZaj),(Xl,Cb,YZak), (Xl,Cb,YZal),(Xl,Cb,YZam),(Xl,Cb,YZan),(Xl,Cb,YZao),(Xl,Cb,YZap),(Xl,Cb,YZaq), (Xl,Cb,YZar),(Xl,Cb,YZas),(Xl,Cb,YZat),(Xl,Cb,YZau),(Xl,Cb,YZav),(Xl,Cb,YZaw), (Xl,Cb,YZax),(Xl,Cb,YZay),(Xl,Cb,YZaz),(Xl,Cb,YZba),(Xl,Cb,YZbb),(Xl,Cb,YZbc), (Xl,Cb,YZbd),(Xl,Cb,YZbe),(Xl,Cb,YZbf),(Xl,Cb,YZbg),(Xl,Cb,YZbh),(Xl,Cb,YZbi), (Xl,Cb,YZbj),(Xl,Cb,YZbk),(Xl,Cb,YZbl),(Xl,Cb,YZbm),(Xl,Cb,YZbn),(Xl,Cb,YZbo), (Xl,Cb,YZbp),(Xl,Cb,YZbq),(Xl,Cb,YZbr),(Xl,Cb,YZbs),(Xl,Cb,YZbt),(Xl,Cb,YZbu), (Xl,Cb,YZbv),(Xl,Cb,YZbw),(Xl,Cb,YZbx),(Xl,Cb,YZby),(Xl,Cb,YZbz),(Xl,Cb, YZca),(Xl,Cb,YZcb),(Xl,Cb,YZcc),(Xl,Cb,YZcd),(Xl,Cb,YZce),(Xl,Cb,YZcf),(Xl,Cb, YZcg),(Xl,Cb,YZch),(Xl,Cb,YZci),(Xl,Cb,YZcj),(Xl,Cb,YZck),(Xl,Cb,YZcl),(Xl,Cb, YZcm),(Xl,Cb,YZcn),(Xl,Cb,YZco),(Xl,Cb,YZcp),(Xl,Cb,YZcq),(Xl,Cb,YZcr),(Xl,Cb, YZcs),(Xl,Cb,YZct),(Xl,Cb,YZcu),(Xl,Cb,YZcv),(Xl,Cb,YZcw),(Xl,Cb,YZcx),(Xl, Cb,YZcy),(Xl,Cb,YZcz),(Xl,Cb,YZda),(Xl,Cb,YZdb),(Xl,Cb,YZdc),(Xl,Cb,YZdd),(Xl, Cb,YZde),(Xl,Cb,YZdf),(Xl,Cb,YZdg),(Xl,Cb,YZdh),(Xl,Cb,YZdi),(Xl,Cb,YZdj),(Xl, Cb,YZdk),(Xl,Cb,YZdl),(Xl,Cb,YZdm),(Xl,Cb,YZdn),(Xl,Cb,YZdo),(Xl,Cb,YZdp),(Xl, Cb,YZdq),(Xl,Cb,YZdr),(Xl,Cb,YZds),(Xl,Cb,Yzdt),(Xl,Cb,YZdu),(Xl,Cb,YZdv),(Xl, Cb,YZdw),(Xl,Cb,YZdx),(Xl,Cb,YZdy),(Xl,Cb,YZdz),(Xl,Cb,YZea),(Xl,Cb,YZeb), (Xl,Cb,YZec),(Xl,Cb,YZed),(Xl,Cb,YZee),(Xl,Cb,YZef),(Xl,Cb,YZeg),(Xl,Cb,YZeh), (Xl,Cb,YZei),(Xl,Cb,YZej),(Xl,Cb,YZek),(Xl,Cb,YZel),(Xl,Cb,YZem),(Xl,Cb,YZen), (Xl,Cb,YZeo),(Xl,Cb,YZep),(Xl,Cb,YZeq),(Xl,Cb,YZer),(Xl,Cb,YZes),(Xl,Cb,YZet), (Xl,Cb,YZeu),(Xl,Cb,YZev),(Xl,Cb,YZew),(Xl,Cb,YZex),(Xl,Cb,YZey),(Xl,Cb,YZez), (Xl,Cb,YZfa),(Xl,Cb,YZfb),(Xl,Cb,YZfc),(Xl,Cb,YZfd),(Xl,Cb,YZfe),(Xl,Cb,YZff), (Xl,Cb,YZfg),(Xl,Cb,YZfh),(Xl,Cb,YZfi),(Xl,Cb,YZfj),(Xl,Cb,YZfk),(Xl,Cb,YZfl),(Xl, Cb,YZfm),(Xl,Cb,YZfn),(Xl,Cb,YZfo),(Xl,Cb,YZfp),(Xl,Cb,YZfq),(Xl,Cb,YZfr),(Xl, Cb,YZfs),(Xl,Cb,YZft),(Xl,Cb,YZful),(Xl,Cb,YZfv),(Xl,Cb,YZfw),(Xl,Cb,YZfx),(Xl, Cb,YZfy),(Xl,Cb,YZfz),(Xl,Cb,YZga),(Xl,Cb,YZgb),(Xl,Cb,YZgc),(Xl,Cb,YZgd),(Xl, Cb,YZge),(Xl,Cb,YZgf),(Xl,Cb,YZgg),(Xl,Cb,YZgh),(Xl,Cb,YZgi),(Xl,Cb,YZgj),(Xl, Cb,YZgk),(Xl,Cb,YZgl),(Xl,Cb,YZgm),(Xl,Cb,YZgn),(Xl,Cb,YZgo),(Xl,Cb,Yzgp),(Xl, Cb,YZgq),(Xl,Cb,YZgr),(Xl,Cb,YZgs),(Xl,Cb,YZgt),(Xl,Cc,YZa),(Xl,Cc,YZb),(Xl, Cc,YZc),(Xl,Cc,YZd),(Xl,Cc,YZe),(Xl,Cc,YZf),(Xl,Cc,YZg),(Xl,Cc,YZh),(Xl,Cc,YZi), (Xl,Cc,YZj),(Xl,Cc,YZk),(Xl,Cc,YZl),(Xl,Cc,YZm),(Xl,Cc,YZn),(Xl,Cc,YZo),(Xl, Cc,YZp),(Xl,Cc,YZq),(Xl,Cc,YZr),(Xl,Cc,YZs),(Xl,Cc,YZt),(Xl,Cc,YZu),(Xl,Cc,YZv), (Xl,Cc,YZw),(Xl,Cc,YZx),(Xl,Cc,YZy),(Xl,Cc,YZz),(Xl,Cc,YZaa),(Xl,Cc,YZab),(Xl, Cc,YZac),(Xl,Cc,YZad),(Xl,Cc,YZae),(Xl,Cc,YZaf),(Xl,Cc,YZag),(Xl,Cc,YZah),(Xl, Cc,YZai),(Xl,Cc,YZaj),(Xl,Cc,YZak),(Xl,Cc,YZal),(Xl,Cc,YZam),(Xl,Cc,YZan),(Xl, Cc,YZao),(Xl,Cc,YZap),(Xl,Cc,YZaq),(Xl,Cc,YZar),(Xl,Cc,YZas),(Xl,Cc,YZat),(Xl,Cc, YZau),(Xl,Cc,YZav),(Xl,Cc,YZaw),(Xl,Cc,YZax),(Xl,Cc,YZay),(Xl,Cc,YZaz),(Xl,Cc, YZba),(Xl,Cc,YZbb),(Xl,Cc,YZbc),(Xl,Cc,YZbd),(Xl,Cc,YZbe),(Xl,Cc,YZbf),(Xl,Cc, YZbg),(Xl,Cc,YZbh),(Xl,Cc,YZbi),(Xl,Cc,YZbj),(Xl,Cc,YZbk),(Xl,Cc,YZbl),(Xl,Cc, YZbm),(Xl,Cc,YZbn),(Xl,Cc,YZbo),(Xl,Cc,YZbp),(Xl,Cc,YZbq),(Xl,Cc,YZbr),(Xl,Cc, YZbs),(Xl,Cc,YZbt),(Xl,Cc,YZbu),(Xl,Cc,YZbv),(Xl,Cc,YZbw),(Xl,Cc,YZbx),(Xl, Cc,YZby),(Xl,Cc,YZbz),(Xl,Cc,YZca),(Xl,Cc,YZcb),(Xl,Cc,YZcc),(Xl,Cc,YZcd),(Xl, Cc,YZce),(Xl,Cc,YZcf),(Xl,Cc,YZcg),(Xl,Cc,YZch),(Xl,Cc,YZei),(Xl,Cc,YZcj),(Xl,Cc, YZck),(Xl,Cc,YZcl),(Xl,Cc,YZcm),(Xl,Cc,YZcn),(Xl,Cc,YZco),(Xl,Cc,YZcp),(Xl,Cc, YZcq),(Xl,Cc,YZcr),(Xl,Cc,YZcs),(Xl,Cc,YZct),(Xl,Cc,YZcu),(Xl,Cc,YZcv),(Xl,Cc, YZcw,),(Xl,Cc,YZcx),(Xl,Cc,YZcy),(Xl,Cc,YZcz),(Xl,Cc,YZda),(Xl,Cc,YZdb),(Xl,Cc, YZdc),(Xl,Cc,YZdd),(Xl,Cc,YZde),(Xl,Cc,YZdf),(Xl,Cc,YZdg),(Xl,Cc,YZdh),(Xl,Cc, YZdi),(Xl,Cc,YZdj),(Xl,Cc,YZdk),(Xl,Cc,YZdl),(Xl,Cc,YZdm),(Xl,Cc,YZdn),(Xl,Cc, YZdo),(Xl,Cc;YZdp),(Xl,Cc,YZdq),(Xl,Cc,YZdr),(Xl,Cc,YZds),(Xl,Cc,Yzdt),(Xl,Cc, YZdu),(Xl,Cc,YZdv),(Xl,Cc,YZdw),(Xl,Cc,YZdx),(Xl,Cc,YZdy),(Xl,Cc,YZdz),(Xl,Cc, YZea),(Xl,Cc,YZeb),(Xl,Cc,YZec),(Xl,Cc,YZed),(Xl,Cc,YZee),(Xl,Cc,YZef),(Xl,Cc, YZeg),(Xl,Cc,YZeh),(Xl,Cc,YZei),(Xl,Cc,YZej),(Xl,Cc,YZek),(Xl,Cc,YZel),(Xl,Cc, YZem),(Xl,Cc,YZen),(Xl,Cc,YZeo),(Xl,Cc,YZep),(Xl,Cc,YZeq),(Xl,Cc,YZer),(Xl,Cc, YZes),(Xl,Cc,YZet),(Xl,Cc,YZeu),(Xl,Cc,YZev),(Xl,Cc,YZew),(Xl,Cc,YZex),(Xl,Cc, YZey),(Xl,Cc,YZez),(Xl,Cc,YZfa),(Xl,Cc,YZfb),(Xl,Cc,YZfc),(Xl,Cc,YZfd),(Xl,Cc,YZfe), (Xl,Cc,YZff),(Xl,Cc,YZfg),(Xl,Cc,YZfh),(Xl,Cc,YZfi),(Xl,Cc,YZfej),(Xl,Cc,YZfk), (Xl,Cc,YZfl),(XICcc,YZfm),(XICcc,YZfn),(Xl,Cc,YZfo),(Xl,Cc,YZfp),(Xl,Cc,YZfq), (Xl,Cc,YZfr),(Xl,Cc,YZfs),(Xl,Cc,YZft),(Xl,Cc,YZfu),(Xi,Cc,yZfv),(Xl,Cc,YZfw),(Xl, Cc,YZfx),(Xl,Cc,YZfY),(Xl,Cc,YZfz),(Xl,Cc,YZga),(Xl,Cc,YZgb),(Xl,Cc,YZgc),(Xl, Cc,YZgd),(Xl,Cc,YZge),(Xl,Cc,YZgf),(Xl,Cc,YZgg),(Xl,Cc,YZgh),(Xl,Cc,YZgi),(Xl, Cc,YZgj),(Xl,Cc,YZgk),(Xl,Cc,YZgl),(Xl,Cc,YZgm),(Xl,Cc,YZgn),(Xl,Cc,YZgo),(Xl, Cc,YZgp),(Xl,Cc,YZgq),(Xl,Cc,YZgr),(Xl,Cc,YZgs),(Xl,Cc,YZgt),(Xl,Cd,YZa),(Xl,Cd, YZb),(Xl,Cd,YZc),(Xl,Cd,YZd),(Xl,Cd,YZe),(Xl,Cd,YZf),(Xl,Cd,YZg),(Xl,Cd,YZh), (Xl,Cd,YZi),(Xl,Cd,YZj),(Xl,Cd,YZk),(Xl,Cd,YZl),(Xl,Cd,YZm),(Xl,Cd,YZn),(Xl,Cd, YZo),(Xl,Cd,YZp),(Xl,Cd,YZq),(Xl,Cd,YZr),(Xl,Cd,YZs),(Xl,Cd,YZt),(Xl,Cd,YZu), (Xl,Cd,YZv),(Xl,Cd,YZw),(Xl,Cd,YZx),(Xl,Cd,YZy),(Xl,Cd,YZz),(Xl,Cd,YZaa),(Xl, Cd,YZab),(Xl,Cd,YZac),(Xl,Cd,YZad),(Xl,Cd,YZae),(Xl,Cd,YZaf),(Xl,Cd,YZag),(Xl, Cd,YZah),(Xl,Cd,YZai),(Xl,Cd,YZaj),(Xl,Cd,YZakc),(Xl,Cd,YZal),(Xl,Cd,YZam),(Xl, Cd,YZan),(Xl,Cd,YZao),(Xl,Cd,YZap),(Xl,Cd,YZaq),(Xl,Cd,YZar),(Xl,Cd,YZas),(Xl, Cd,YZat),(Xl,Cd,YZau),(Xl,Cd,YZav),(Xl,Cd,YZaw),(Xl,Cd,YZax),(Xl,Cd,YZay),(Xl, Cd,YZaz),(Xl,Cd,YZba),(Xl,Cd,YZbb),(Xl,Cd,YZbc),(Xl,Cd,YZbd),(Xl,Cd,YZbe),(Xl, Cd,YZbf),(Xl,Cd,YZbg),(Xl,Cd,YZbh),(Xl,Cd,YZbi),(Xl,Cd,YZbj),(Xl,Cd,YZbk),(Xl, Cd,YZbl),(Xl,Cd,YZbm),(Xl,Cd,YZbn),(Xl,Cd,YZbo),(Xl,Cd,Y;Zbp),(Xl,Cd,YZbq),(Xl, Cd,YZbr),(Xl,Cd,YZbs),(Xl,Cd,YZbt),(Xl,Cd,YZbu),(Xl,Cd,YZbv),(Xl,Cd,YZbw), (Xl,Cd,YZbx),(Xl,Cd,YZby),(Xl,Cd,YZbz),(Xl,Cd,YZca),(Xl,Cd,YZeb),(Xl,Cd,YZcc), (Xl,Cd,YZcd),(Xl,Cd,YZce),(Xl,Cd,YZcf),(Xl,Cd,YZcg),(Xl,Cd,YZch),(Xl,Cd,YZci), (Xl,Cd,YZcj),(Xl,Cd,YZck),(Xl,Cd,YZcl),(Xl,Cd,YZcm),(Xl,Cd,YZcn),(Xl,Cd,YZco), (Xl,Cd,YZcp),(Xl,Cd,YZcq),(Xl,Cd,YZcr),(Xl,Cd,YZcs),(Xl,Cd,YZct),(Xl,Cd,YZcu),(Xl, Cd,YZcv),(Xl,Cd,YZcw),(Xl,Cd,YZcx),(Xl,Cd,YZcy),(Xl,Cd,YZcz),(Xl,Cd,YZda), (Xl,Cd,YZdb),(Xl,Cd,YZdc),(Xl,Cd,YZdd),(Xl,Cd,YZde),(Xl,Cd,YZdf),(Xl,Cd,YZdg), (Xl,Cd,YZdh),(Xl,Cd,YZdi),(Xl,Cd,YZdj),(Xl,Cd,YZdk),(Xl,Cd,YZdl),(Xl,Cd,YZdm), (Xl,Cd,YZdn),(Xl,Cd,YZdo),(Xl,Cd,YZdp),(Xl,Cd,YZdq),(Xl,Cd,YZdr),(Xl,Cd,YZds), (Xl,Cd,Yzdt),(Xl,Cd,YZdu),(Xl,Cd,YZdv),(Xl,Cd,YZdw),(Xl,Cd,YZdx),(Xl,Cd,YZdy), (Xl,Cd,YZdz),(Xl,Cd,YZea),(Xl,Cd,YZeb),(Xl,Cd,YZec),(Xl,Cd,YZed),(Xl,Cd,YZee), (Xl,Cd,YZef),(Xl,Cd,YZeg),(Xl,Cd,YZeh),(Xl,Cd,YZei),(Xl,Cd,YZej),(Xl,Cd,YZek), (Xl,Cd,YZel),(Xl,Cd,YZem),(Xl,Cd,YZen),(Xl,Cd,YZeo),(Xl,Cd,YZep),(Xl,Cd, YZeq),(Xl,Cd,YZer),(Xl,Cd,YZes),(Xl,Cd,YZet),(Xl,Cd,YZeu),(Xl,Cd,YZev),(Xl,Cd, YZew),(Xl,Cd,YZex),(Xl,Cd,YZey),(Xl,Cd,YZez),(Xl,Cd,YZfa),(Xl,Cd,YZfb),(Xl,Cd, YZfc),(Xl,Cd,YZfd),(Xl,Cd,YZfe),(Xl,Cd,YZff),(Xl,Cd,YZfg),(Xl,Cd,YZfh),(Xl,Cd, YZfi),(Xl,Cd,YZfj),(Xl,Cd,YZfk),(Xl,Cd,YZfl),(Xl,Cd,YZfmn),(Xl,Cd,YZfn),(Xl,Cd,YZfo), (Xl,Cd,YZfp),(Xl,Cd,YZfq),(Xl,Cd,YZfr),(Xl,Cd,YZfs),(Xl,Cd,YZft),(Xl,Cd,YZfu), (Xl,Cd,YZfv),(Xl,Cd,YZfw),(Xl,Cd,YZfx),(Xl,Cd,YZfy),(Xl,Cd,YZfz),(Xl,Cd,YZga), (Xl,Cd,YZgb),(Xl,Cd,YZgc),(Xl,Cd,YZgd),(Xl,Cd,YZge),(Xl,Cd,YZgf),(Xl,Cd,YZgg), (Xl,Cd,YZgh),(Xl,Cd,YZgi),(Xl,Cd,YZgj),(Xl,Cd,YZgk),(Xl,Cd,YZgl),(Xl,Cd,YZgm), (Xl,Cd,YZgn),(Xl,Cd,YZgo),(Xl,Cd,YZgp),(Xl,Cd,YZgq),(Xl,Cd,YZgr),(Xl,Cd,YZgs), (Xl,Cd,YZgt),(Xl,Ce,YZa),(Xl,Ce,YZb),(Xl,Ce,YZc),(Xl,Ce,YZd),(Xl,Ce,YZe), (Xl,Ce,YZf),(Xl,Ce,YZg),(Xl,Ce,YZh),(Xl,Ce,YZi),(Xl,Ce,YZj),(Xl,Ce,YZk),(Xl,Ce,YZl), (Xl,Ce,YZm),(Xl,Ce,YZn),(Xl,Ce,YZo),(Xl,Ce,YZp),(Xl,Ce,YZq),(Xl,Ce,YZr),(Xl, Ce,YZs),(Xl,Ce,YZt),(Xl,Ce,YZu),(Xl,Ce,YZv),(Xl,Ce,YZw),(Xl,Ce,YZx),(Xl,Ce,YZy), (Xl,Ce,YZz),(Xl,Ce,YZaa),(Xl,Ce,YZab),(Xl,Ce,YZac),(Xl,Ce,YZad),(Xl,Ce,YZae), (Xl,Ce,YZaf),(Xl,Ce,YZag),(Xl,Ce,YZah),(Xl,Ce,YZai),(Xl,Ce,YZaj),(Xl,Ce,YZak), (Xl,Ce,YZal),(Xl,Ce,YZam),(Xl,Ce,YZan),(Xl,Ce,YZao),(Xl,Ce,YZap),(Xl,Ce,YZaq), (Xl,Ce,YZar),(Xl,Ce,YZas),(Xl,Ce,YZat),(Xl,Ce,YZau),(Xl,Ce,YZav),(Xl,Ce,YZaw), (Xl,Ce,YZax),(Xl,Ce,YZay),(Xl,Ce,YZaz),(Xl,Ce,YZba),(Xl,Ce,YZbb),(Xl,Ce,YZbc), (Xl,Ce,YZbd),(Xl,Ce,YZbe),(Xl,Ce,YZbf),(Xl,Ce,YZbg),(Xl,Ce,YZbh),(Xl,Ce,YZbi), (Xl,Ce,YZbj),(Xl,Ce,YZbk),(Xl,Ce,YZbl),(Xl,Ce,YZbm),(Xl,Ce,YZbn),(Xl,Ce,YZbo), (Xl,Ce,YZbp),(Xl,Ce,YZbq),(Xl,Ce,YZbr),(Xl,Ce,YZbs),(Xl,Ce,YZbt),(Xl,Ce,YZbu), (Xl,Ce,YZbv),(Xl,Ce,YZbw),(Xl,Ce,YZbx),(Xl,Ce,YZby),(Xl,Ce,YZbz),(Xl,Ce,YZca), (Xl,Ce,YZcb),(Xl,Ce,YZcc),(Xl,Ce,YZcd),(Xl,Ce,YZce),(Xl,Ce,YZcf),(Xl,Ce,YZcg), (Xl,Ce,YZch),(Xl,Ce,YZci),(Xl,Ce,YZcj),(Xl,Ce,YZck),(Xl,Ce,YZcl),(Xl,Ce,YZcm), (Xl,Ce,YZcn),(Xl,Ce,YZco),(Xl,Ce,YZcp),(Xl,Ce,YZcq),(Xl,Ce,YZcr),(Xl,Ce,YZcs), (Xl,Ce,YZct),(Xl,Ce,YZcu),(Xl,Ce,YZcv),(Xl,Ce,YZcw),(Xl,Ce,YZcx),(Xl,Ce,YZcy), (Xl,Ce,YZcz),(Xl,Ce,YZda),(Xl,Ce,YZdb),(Xl,Ce,YZdc),(Xl,Ce,YZdd),(Xl,Ce,YZde), (Xl,Ce,YZdf),(Xl,Ce,YZdg),(Xl,Ce,YZdh),(Xl,Ce,YZdi),(Xl,Ce,YZdj),(Xl,Ce,YZdk), (Xl,Ce,YZdl),(Xl,Ce,YZdm),(Xl,Ce,YZdn),(Xl,Ce,YZdo),(Xl,Ce,YZdp),(Xl,Ce,YZdq), (Xl,Ce,YZdr),(Xl,Ce,YZds),(Xl,Ce,YZdt),(Xl,Ce,YZdu),(Xl,Ce,YZdv),(Xl,Ce,YZdw), (Xl,Ce,YZdx),(Xl,Ce,YZdy),(Xl,Ce,YZdz),(Xl,Ce,YZea),(Xl,Ce,YZeb),(Xl,Ce,YZec), (Xl,Ce,YZed),(Xl,Ce,YZee),(Xl,Ce,YZef),(Xl,Ce,YZeg),(Xl,Ce,YZeh),(Xl,Ce,YZei), (Xl,Ce,YZej),(Xl,Ce,YZek),(Xl,Ce,YZel),(Xl,Ce,YZem),(Xl,Ce,YZen),(Xl,Ce,YZeo), (Xl,Ce,YZep),(Xl,Ce,YZeq),(Xl,Ce,YZer),(Xl,Ce,YZes),(Xl,Ce,YZet),(Xl,Ce,YZeu), (Xl,Ce,YZev),(Xl,Ce,YZew),(Xl,Ce,YZex),(Xl,Ce,YZey),(Xl,Ce,YZez),(Xl,Ce,YZfa), (Xl,Ce,YZfb),(Xl,Ce,YZfc),(Xl,Ce,YZfd),(Xl,Ce,YZfe),(Xl,Ce,YZff),(Xl,Ce,YZfg),(Xl, Ce,YZfh),(Xl,Ce,YZfi),(Xl,Ce,YZfj),(Xl,Ce,YZfk),(Xl,Ce,YZfl),(Xl,Ce,YZfm),(Xl,Ce, YZfn),(Xl,Ce,YZfo),(Xl,Ce,YZfp),(Xl,Ce,YZfq),(Xl,Ce,YZfr),(Xl,Ce,YZfs),(Xl,Ce, YZft),(Xl,Ce,YZfu),(Xl,Ce,YZfv),(Xl,Ce,YZfw),(Xl,Ce,YZfx),(Xl,Ce,YZfy),(Xl,Ce,YZfz), (Xl,Ce,YZga),(Xl,Ce,YZgb),(Xl,Ce,YZgc),(Xl,Ce,YZgd),(Xl,Ce,YZge),(Xl,Ce,YZgf), (Xl,Ce,YZgg),(Xl,Ce,YZgh),(Xl,Ce,YZgi),(Xl,Ce,YZgj),(Xl,Ce,YZgk),(Xl,Ce,YZgl), (Xl,Ce,YZgm),(Xl,Ce,YZgn),(Xl,Ce,YZgo),(Xl,Ce,YZgp),(Xl,Ce,YZgq),(Xl,Ce, YZgr),(Xl,Ce,YZgs),(Xl,Ce,YZgt),(Xl,Cf,YZa),(Xl,Cf,YZb),(Xl,Cf,YZc),(Xl,Cf,YZd), (Xl,Cf,YZe),(Xl,Cf,YZf),(Xl,Cf,YZg),(Xl,Cf,YZh),(Xl,Cf,YZi),(Xl,Cf,YZj),(Xl,Cf,YZk), (Xl,Cf,YZl),(Xl,Cf,YZm),(X,Cf,YZn),(Xl,Cf,YZo),(Xl,Cf,YZp),(Xl,Cf,YZq),(Xl,Cf, YZr),(Xl,Cf,YZs),(Xl,Cf,YZt),(Xl,Cf,YZu),(Xl,Cf,YZv),(Xl,Cf,YZw),(Xl,Cf,YZx), (Xl,Cf,YZy),(Xl,Cf,YZz),(Xl,Cf,YZaa),(Xl,Cf,YZab),(Xl,Cf,YZac),(Xl,Cf,YZad),(Xl, Cf,YZae),(Xl,Cf,YZaf),(Xl,Cf,YZag),(Xl,Cf,YZah),(Xl,Cf,YZai),(Xl,Cf,YZaj),(Xl,Cf, YZak),(Xl,Cf,YZal),(Xl,Cf,YZam),(Xl,Cf,YZan),(Xl,Cf,YZao),(Xl,Cf,YZap),(Xl,Cf,YZaq), (Xl,Cf,YZar),(Xl,Cf,YZas),(Xl,Cf,YZat),(Xl,Cf,YZau),(Xl,Cf,YZav),(Xl,Cf,YZaw), (Xl,Cf,YZax),(Xl,Cf,YZay),(Xl,Cf,YZaz),(Xl,Cf,YZba),(Xl,Cf,YZbb),(Xl,Cf,YZbc), (Xl,Cf,YZbd),(Xl,Cf,YZbe),(Xl,Cf,YZbf),(Xl,Cf,YZbg),(Xl,Cf,YZbh),(Xl,Cf,YZbi),(Xl, Cf,YZbj),(Xl,Cf,YZbk),(Xl,Cf,YZbl),(Xl,Cf,YZbm),(Xl,Cf,YZbn),(Xl,Cf,YZbo),(Xl, Cf,YZbp),(Xl,Cf,YZbq),(Xl,Cf,YZbr),(Xl,Cf,YZbs),(Xl,Cf,YZbt),(Xl,Cf,YZbu),(Xl, Cf,YZbv),(Xl,Cf,YZbw),(Xl,Cf,YZbx),(Xl,Cf,YZby),(Xl,Cf,YZbz),(Xl,Cf,YZca),(Xl, Cf,YZcb),(Xl,Cf,YZcc),(Xl,Cf,YZcd),(Xl,Cf,YZce),(Xl,Cf,YZcf),(Xl,Cf,YZcg),(Xl,Cf, YZch),(Xl,Cf,YZci),(Xl,Cf,YZcj),(Xl,Cf,YZck),(Xl,Cf,YZcl),(Xl,Cf,YZcm),(Xl,Cf,YZcn), (Xl,Cf,YZco),(Xl,Cf,YZcp),(Xl,Cf,YZcq),(Xl,Cf,YZcr),(Xl,Cf,YZcs),(Xl,Cf,YZct), (Xl,Cf,YZcu),(Xl,Cf,YZcv),(Xl,Cf,YZcw),(Xl,Cf,YZcx),(Xl,Cf,YZcy),(Xl,Cf,YZcz), (Xl,Cf,YZda),(Xl,Cf,YZdb),(Xl,Cf,YZdc),(Xl,Cf,YZdd),(Xl,Cf,YZde),(Xl,Cf,YZdf),(Xl, Cf,YZdg),(Xl,Cf,YZdh),(Xl,Cf,YZdi),(Xl,Cf,YZdj),(Xl,Cf,YZdk),(Xl,Cf,YZdl),(Xl, Cf,YZdm),(Xl,Cf,YZdn),(Xl,Cf,YZdo),(Xl,Cf,YZdp),(Xl,Cf,YZdq),(Xl,Cf,YZdr),(Xl, Cf,YZds),(Xl,Cf,YZdt),(Xl,Cf,YZdu),(Xl,Cf,YZdv),(Xl,Cf,YZdw),(Xl,Cf,YZdx),(Xl,Cf, YZdy),(Xl,Cf,YZdz),(Xl,Cf,YZea),(Xl,Cf,YZeb),(Xl,Cf,YZec),(Xl,Cf,YZed),(Xl,Cf, YZee),(Xl,Cf,YZef),(Xl,Cf,YZeg),(Xl,Cf,YZeh),(Xl ,Cf,YZei),(Xl,Cf,YZej),(Xl,Cf,YZek), (Xl,Cf,YZel),(Xl,Cf,YZem),(Xl,Cf,YZen),(Xl,Cf,YZeo),(Xl,Cf,YZep),(Xl,Cf,YZeq), (Xl,Cf,YZer),(Xl,Cf,YZes),(Xl,Cf,YZet),(Xl,Cf,YZeu),(Xl,Cf,YZev),(Xl,Cf,YZew), (Xl,Cf,YZex),(Xl,Cf,YZey),(Xl,Cf,YZez),(Xl,Cf,YZfa),(Xl,Cf,YZfb),(Xl,Cf,YZfc),(Xl, Cf,YZfd),(Xl,Cf,YZfe),(Xl,Cf,YZff),(Xl,Cf,YZfg),(Xl,Cf,YZfh),(Xl,Cf,YZfi),(Xl,Cf, YZfj),(Xl,Cf,YZfk),(Xl,Cf,YZfl),(Xl,Cf,YZfm),(Xl,Cf,YZfn),(Xl,Cf,YZfo),(Xl,Cf,YZfp), (Xl,Cf,YZfq),(Xl,Cf,YZfr),(Xl,Cf,YZfs),(Xl,Cf,YZft),(Xl,Cf,YZfu),(Xl,Cf,YZfv),(Xl, Cf,YZfw),(Xl,Cf,YZfx),(Xl,Cf,YZfy),(Xl,Cf,YZfz),(Xl,Cf,YZga),(Xl,Cf,YZgb),(Xl, Cf,YZgc),(Xl,Cf,YZgd),(Xl,Cf,YZge),(Xl,Cf,YZgf),(Xl,Cf,YZgg),(Xl,Cf,YZgh),(Xl, Cf,YZgi),(Xl,Cf,YZgj),(Xl,Cf,YZgk),(Xl,Cf,YZgl),(Xl,Cf,YZgm),(Xl,Cf,YZgn),(Xl,Cf, YZgo),(Xl,Cf,YZgp),(Xl,Cf,YZgq),(Xl,Cf,YZgr),(Xl,Cf,YZgs),(Xl,Cf,YZgt), (Xm,Ca,YZa),(Xm,Ca,YZb),(Xm,Ca,YZc),(Xm,Ca,YZd),(Xm,Ca,YZe),(Xm,Ca,YZf), (Xm,Ca,YZg),(Xm,Ca,YZh),(Xm,Ca,YZi),(Xm,Ca,YZj),(Xm,Ca,YZk),(Xm,Ca,YZl),(Xm, Ca,YZm),(Xm,Ca,YZn),(Xm,Ca,YZo),(Xm,Ca,YZp),(Xm,Ca,YZq),(Xm,Ca,YZr), (Xm,Ca,YZs),(Xm,Ca,YZt),(Xm,Ca,YZu),(YXm,Ca,YZv),(Xm,Ca,YZw),(Xm,Ca,YZx), (Xm,Ca,YZy),(Xm,Ca,YZz),(Xm,Ca,YZaa),(Xm,Ca,YZab),(Xm,Ca,YZac),(Xm,Ca,YZad), (Xm,Ca,YZae),(Xm,Ca,YZaf),(Xm,Ca,YZag),(Xm,Ca,YZah),(Xm,Ca,YZai),(Xm, Ca,YZaj),(Xm,Ca,YZak),(Xm,Ca,YZal),(Xm,Ca,YZam),(YXm,Ca,YZan),(Xm,Ca,YZao), (Xm,Ca,YZap),(Xm,Ca,YZaq),(Xm,Ca,YZar),(Xm,Ca,YZas),(Xm,Ca,YZat),(Xm, Ca,YZau),(Xm,Ca,YZav),(Xm,Ca,YZaw),(Xm,Ca,YZax),(Xm,Ca,YZay),(Xm,Ca,YZaz), (Xm,Ca,YZba),(Xm,Ca,YZbb),(Xm,Ca,YZbc),(Xm,Ca,YZbd),(Xm,Ca,YZbe),(Xm, Ca,YZbf),(Xm,Ca,YZbg),(Xm,Ca,YZbh),(Xm,Ca,YZbi),(Xm,Ca,YZbj),(Xm,Ca,YZbk), (Xm,Ca,YZbl),(Xm,Ca,YZbm),(Xm,Ca,YZbn),(Xm,Ca,YZbo),(Xm,Ca,YZbp),(Xm, Ca,YZbq),(Xm,Ca,YZbr),(Xm,Ca,YZbs),(Xm,Ca,YZbt),(Xm,Ca,YZbu),(Xm,Ca,YZbv), (Xm,Ca,YZbw),(Xm,Ca,YZbx),(Xm,Ca,YZby),(Xm,Ca,YZbz),(Xm,Ca,YZca),(Xm, Ca,YZcb),(Xm,Ca,YZcc),(Xm,Ca,YZcd),(Xm,Ca,YZce),(Xm,Ca,YZcf),(Xm,Ca,YZcg), (Xm,Ca,YZch),(Xm,Ca,YZci),(Xm,Ca,YZcj),(Xm,Ca,YZck),(Xm,Ca,YZcl),(Xm,Ca, YZcm),(Xm,Ca,YZcn),(Xm,Ca,YZco),(Xm,Ca,YZcp),(Xm,Ca,YZcq),(Xm,Ca,YZcr), (Xm,Ca,YZcs),(Xm,Ca,YZct),(Xm,Ca,YZcu),(Xm,Ca,YZcv),(Xm,Ca,YZcw),(Xm,Ca, YZcx),(Xm,Ca,YZcy),(Xm,Ca,YZcz),(Xm,Ca,YZda),(Xm,Ca,YZdb),(Xm,Ca,YZdc),(Xm, Ca,YZdd),(Xm,Ca,YZde),(Xm,Ca,YZdf),(Xm,Ca,YZdg),(Xm,Ca,YZdh),(Xm,Ca, YZdi),(Xm,Ca,YZdj),(Xm,Ca,YZdk),(Xm,Ca,YZdl),(Xm,Ca,YZdm),(Xm,Ca,YZdn),(Xm, Ca,YZdo),(Xm,Ca,YZdp),(Xm,Ca,YZdq),(Xm,Ca,YZdr),(Xm,Ca,YZds),(Xm,Ca, YZdt),(Xm,Ca,YZdu),(Xm,Ca,YZdv),(Xm,Ca,YZdw),(Xm,Ca,YZdx),(Xm,Ca,YZdy),(Xm, Ca,YZdz),(Xm,Ca,YZea),(Xm,Ca,YZeb),(Xm,Ca,YZec),(Xm,Ca,YZed),(Xm,Ca, YZee),(Xm,Ca,YZef),(Xm,Ca,YZeg),(Xm,Ca,YZeh),(Xm,Ca,YZei),(Xm,Ca,YZej),(Xm, Ca,YZek),(Xm,Ca,YZel),(Xm,Ca,YZem),(Xm,Ca,YZen),(Xm,Ca,YZeo),(Xm,Ca,YZep), (Xm,Ca,YZeq),(Xm,Ca,YZer),(Xm,Ca,YZes),(Xm,Ca,YZet),(Xm,Ca,YZeu),(Xm, Ca,YZev),(Xm,Ca,YZew),(Xm,Ca,YZex),(Xm,Ca,YZey),(Xm,Ca,YZez),(Xm,Ca,YZfa), (Xm,Ca,YZfb),(Xm,Ca,YZfc),(Xm,Ca,YZfd),(Xm,Ca,YZfe),(Xm,Ca,YZff),(Xm,Ca, YZfg),(Xm,Ca,YZfh),(Xm,Ca,YZfi),(Xm,Ca,YZfj),(Xm,Ca,YZfk),(Xm,Ca,YZfl),(Xm, Ca,YZfm),(Xm,Ca,YZfn),(Xm,Ca,YZfo),(Xm,Ca,YZfp),(Xm,Ca,YZfq),(Xm,Ca,YZfr), (Xm,Ca,YZfs),(Xm,Ca,YZft),(Xm,Ca,YZfu),(Xm,Ca,YZfv),(Xm,Ca,YZfw),(Xm,Ca, YZfx),(Xm,Ca,YZfy),(Xm,Ca,YZfz),(Xm,Ca,YZga),(Xm,Ca,YZgb),(Xm,Ca,YZgc), (Xm,Ca,YZgd),(Xm,Ca,YZge),(Xm,Ca,YZgf),(Xm,Ca,YZgg),(Xm,Ca,YZgh),(Xm,Ca, YZgi),(Xm,Ca,YZgj),(Xm,Ca,YZgk),(Xm,Ca,YZgl),(Xm,Ca,YZgm),(Xm,Ca,YZgn),(Xm, Ca,YZgo),(Xm,Ca,YZgp),(Xm,Ca,YZgq),(Xm,Ca,YZgr),(Xm,Ca,YZgs),(Xm,Ca, YZgt),(Xm,Cb,YZa),(Xm,Cb,YZb),(Xm,Cb,YZc),(Xm,Cb,YZd),(Xm,Cb,YZe),(Xm,Cb, YZf),(Xm,Cb,YZg),(Xm,Cb,YZh),(Xm,Cb,YZi),(Xm,Cb,YZj),(Xm,Cb,YZk),(Xm,Cb, YZl),(Xm,Cb,YZm),(Xm,Cb,YZn),(Xm,Cb,YZo),(Xm,Cb,YZp),(Xm,Cb,YZq),(Xm, Cb,YZr),(Xm,Cb,YZs),(Xm,Cb,YZt),(Xm,Cb,YZu),(Xm,Cb,YZv),(Xm,Cb,YZw),(Xm, Cb,YZx),(Xm,Cb,YZy),(Xm,Cb,YZz),(Xm,Cb,YZaa),(Xm,Cb,YZab),(Xm,Cb,YZac), (Xm,Cb,YZad),(Xm,Cb,YZae),(Xm,Cb,YZaf),(Xm,Cb,YZag),(Xm,Cb,YZah),(Xm,Cb, YZai),(Xm,Cb,YZaj),(Xm,Cb,YZak),(Xm,Cb,YZal),(Xm,Cb,YZam),(Xm,Cb,YZan),(Xm, Cb,YZao),(Xm,Cb,YZap),(Xm,Cb,YZaq),(Xm,Cb,YZar),(Xm,Cb,YZas),(Xm,Cb, YZat),(Xm,Cb,YZau),(Xm,Cb,YZav),(Xm,Cb,YZaw),(Xm,Cb,YZax),(Xm,Cb,YZay),(Xm, Cb,YZaz),(Xm,Cb,YZba),(Xm,Cb,YZbb),(Xm,Cb,YZbc),(Xm,Cb,YZbd),(Xm,Cb, YZbe),(Xm,Cb,YZbf),(Xm,Cb,YZbg),(Xm,Cb,YZbh),(Xm,Cb,YZbi),(Xm,Cb,YZbj),(Xm, Cb,YZbk),(Xm,Cb,YZbl),(Xm,Cb,YZbm),(Xm,Cb,YZbn),(Xm,Cb,YZbo),(Xm,Cb, YZbp),(Xm,Cb,YZbq),(Xm,Cb,YZbr),(Xm,Cb,YZbs),(Xm,Cb,YZbt),(Xm,Cb,YZbu), (Xm,Cb,YZbv),(Xm,Cb,YZbw),(Xm,Cb,YZbx),(Xm,Cb,YZby),(Xm,Cb,YZbz),(Xm, Cb,YZca),(Xm,Cb,YZcb),(Xm,Cb,YZcc),(Xm,Cb,YZcd),(Xm,Cb,YZce),(Xm,Cb,YZcf), (Xm,Cb,YZcg),(Xm,Cb,YZch),(Xm,Cb,YZci),(Xm,Cb,YZcj),(Xm,Cb,YZck),(Xm, Cb,YZcl),(Xm,Cb,YZcm),(Xm,Cb,YZcn),(Xm,Cb,YZco),(Xm,Cb,YZcp),(Xm,Cb,YZcq), (Xm,Cb,YZcr),(Xm,Cb,YZcs),(Xm,Cb,YZct),(Xm,Cb,YZcu),(Xm,Cb,YZcv),(Xm, Cb,YZcw),(Xm,Cb,YZcx),(Xm,Cb,YZcy),(Xm,Cb,YZcz),(Xm,Cb,YZda),(Xm,Cb,YZdb), (Xm,Cb,YZdc),(Xm,Cb,YZdd),(Xm,Cb,YZde),(Xm,Cb,YZdf),(Xm,Cb,YZdg),(Xm, Cb,YZdh),(Xm,Cb,YZdi),(Xm,Cb,YZdj),(Xm,Cb,YZdk),(Xm,Cb,YZdl),(Xm,Cb,YZdm), (Xm,Cb,YZdn),(Xm,Cb,YZdo),(Xm,Cb,YZdp),(Xm,Cb,YZdq),(Xm,Cb,YZdr),(Xm, Cb,YZds),(Xm,Cb,YZdt),(Xm,Cb,YZdu),(Xm,Cb,YZdv),(Xm,Cb,YZdw),(Xm,Cb, YZdx),(Xm,Cb,YZdy),(Xm,Cb,YZdz),(Xm,Cb,YZea),(Xm,Cb,YZeb),(Xm,Cb,YZec),(Xm, Cb,YZed),(Xm,Cb,YZee),(Xm,Cb,YZef),(Xm,Cb,YZeg),(Xm,Cb,YZeh),(Xm,Cb, YZei),(Xm,Cb,YZej),(Xm,Cb,YZek),(Xm,Cb,YZel),(Xm,Cb,YZem),(Xm,Cb,YZen),(Xm, Cb,YZeo),(Xm,Cb,YZep),(Xm,Cb,YZeq),(Xm,Cb,YZer),(Xm,Cb,YZes),(Xm,Cb, YZet),(Xm,Cb,YZeu),(Xm,Cb,YZev),(Xm,Cb,YZew),(Xm,Cb,YZex),(Xm,Cb,YZey),(Xm, Cb,YZez),(Xm,Cb,YZfa),(Xm,Cb,YZfb),(Xm,Cb,YZfc),(Xm,Cb,YZfd),(Xm,Cb, YZfe),(Xm,Cb,YZff),(Xm,Cb,YZfg),(Xm,Cb,YZfh),(Xm,Cb,YZfi),(Xm,Cb,YZfj),(Xm, Cb,YZfk),(Xm,Cb,YZfl),(Xm,Cb,YZfm),(Xm,Cb,YZfn),(Xm,Cb,YZfo),(Xm,Cb,YZfp), (Xm,Cb,YZfq),(Xm,Cb,YZfr),(Xm,Cb,YZfs),(Xm,Cb,YZft),(Xm,Cb,YZfu),(Xm, Cb,YZfv),(Xm,Cb,YZfw),(Xm,Cb,YZfx),(Xm,Cb,YZfy),(Xm,Cb,YZfz),(Xm,Cb,YZga), (Xm,Cb,YZgb),(Xm,Cb,YZgc),(Xm,Cb,YZgd),(Xm,Cb,YZge),(Xm,Cb,YZgf),(Xm, Cb,YZgg),(Xm,Cb,YZgh),(Xm,Cb,YZgi),(Xm,Cb,YZgj),(Xm,Cb,YZgk),(Xm,Cb,YZgl), (Xm,Cb,YZgm),(Xm,Cb,YZgn),(Xm,Cb,YZgo),(Xm,Cb,YZgp),(Xm,Cb,YZgq),(Xm, Cb,YZgr),(Xm,Cb,YZgs),(Xm,Cb,YZgt),(Xm,Cc,YZa),(Xm,Cc,YZb),(Xm,Cc,YZc), (Xm,Cc,YZd),(Xm,Cc,YZe),(Xm,Cc,YZf),(Xm,Cc,YZg),(Xm,Cc,YZh),(Xm,Cc,YZi), (Xm,Cc,YZj),(Xm,Cc,YZk),(Xm,Cc,YZl),(Xm,Cc,YZm),(Xm,Cc,YZn),(Xm,Cc,YZo), (Xm,Cc,YZp),(Xm,Cc,YZq),(Xm,Cc,YZr),(Xm,Cc,YZs),(Xm,Cc,YZt),(Xm,Cc,YZu),(Xm, Cc,YZv),(Xm,Cc,YZw),(Xm,Cc,YZx),(Xm,Cc,YZy),(Xm,Cc,YZz),(Xm,Cc,YZaa), (Xm,Cc,YZab),(Xm,Cc,YZac),(Xm,Cc,YZad),(Xm,Cc,YZae),(Xm,Cc,YZaf),(Xm,Cc, YZag),(Xm,Cc,YZah),(Xm,Cc,YZai),(Xm,Cc,YZaj),(Xm,Cc,YZak),(Xm,Cc,YZal),(Xm, Cc,YZam),(Xm,Cc,YZan),(Xm,Cc,YZao),(Xm,Cc,YZap),(Xm,Cc,YZaq),(Xm,Cc, YZar),(Xm,Cc,YZas),(Xm,Cc,YZat),(Xm,Cc,YZau),(Xm,Cc,YZav),(Xm,Cc,YZaw),(Xm, Cc,YZax),(Xm,Cc,YZay),(Xm,Cc,YZaz),(Xm,Cc,YZba),(Xm,Cc,YZbb),(Xm,Cc, YZbc),(Xm,Cc,YZbd),(Xm,Cc,YZbe),(Xm,Cc,YZbf),(Xm,Cc,YZbg),(Xm,Cc,YZbh),(Xm, Cc,YZbi),(Xm,Cc,YZbj),(Xm,Cc,YZbk),(Xm,Cc,YZbl),(Xm,Cc,YZbm),(Xm,Cc, YZbn),(Xm,Cc,YZbo),(Xm,Cc,YZbp),(Xm,Cc,YZbq),(Xm,Cc,YZbr),(Xm,Cc,YZbs),(Xm, Cc,YZbt),(Xm,Cc,YZbu),(Xm,Cc,YZbv),(Xm,Cc,YZbw),(Xm,Cc,YZbx),(Xm,Cc, YZby),(Xm,Cc,YZbz),(Xm,Cc,YZca),(Xm,Cc,YZcb),(Xm,Cc,YZcc),(Xm,Cc,YZcd),(Xm, Cc,YZce),(Xm,Cc,YZcf),(Xm,Cc,YZcg),(Xm,Cc,YZch),(Xm,Cc,YZci),(Xm,Cc,YZcj), (Xm,Cc,YZck),(Xm,Cc,YZcl),(Xm,Cc,YZcm),(Xm,Cc,YZcn),(Xm,Cc,YZco),(Xm, Cc,YZcp),(Xm,Cc,YZcq),(Xm,Cc,YZcr),(Xm,Cc,YZcs),(Xm,Cc,YZct),(Xm,Cc,YZcu), (Xm,Cc,YZcv),(Xm,Cc,YZcw),(Xm,Cc,YZcx),(Xm,Cc,YZcy),(Xm,Cc,YZcz),(Xm, Cc,YZda),(Xm,Cc,YZdb),(Xm,Cc,YZdc),(Xm,Cc,YZdd),(Xm,Cc,YZde),(Xm,Cc,YZdf), (Xm,Cc,YZdg),(Xm,Cc,YZdh),(Xm,Cc,YZdi),(Xm,Cc,YZdj),(Xm,Cc,YZdk),(Xm, Cc,YZdl),(Xm,Cc,YZdm),(Xm,Cc,YZdn),(Xm,Cc,YZdo),(Xm,Cc,YZdp),(Xm,Cc,YZdq), (Xm,Cc,YZdr),(Xm,Cc,YZds),(Xm,Cc,YZdt),(Xm,Cc,YZdu),(Xm,Cc,YZdv),(Xm, Cc,YZdw),(Xm,Cc,YZdx),(Xm,Cc,YZdy),(Xm,Cc,YZdz),(Xm,Cc,YZea),(Xm,Cc,YZeb), (Xm,Cc,YZec),(Xm,Cc,YZed),(Xm,Cc,YZee),(Xm,Cc,YZef),(Xm,Cc,YZeg),(Xm, Cc,YZeh),(Xm,Cc,YZei),(Xm,Cc,YZej),(Xm,Cc,YZek),(Xm,Cc,YZel),(Xm,Cc,YZem), (Xm,Cc,YZen),(Xm,Cc,YZeo),(Xm,Cc,YZep),(Xm,Cc,YZeq),(Xm,Cc,YZer),(Xm, Cc,YZes),(Xm,Cc,YZet),(Xm,Cc,YZeu),(Xm,Cc,YZev),(Xm,Cc,YZew),(Xm,Cc,YZex), (Xm,Cc,YZey),(Xm,Cc,YZez),(Xm,Cc,YZfa),(Xm,Cc,YZfb),(Xm,Cc,YZfc),(Xm,Cc, YZfd),(Xm,Cc,YZfe),(Xm,Cc,YZff),(Xm,Cc,YZfg),(Xm,Cc,YZfh),(Xm,Cc,YZfi),(Xm, Cc,YZfj),(Xm,Cc,YZfk),(Xm,Cc,YZfl),(Xm,Cc,YZfm),(Xm,Cc,YZfn),(Xm,Cc,YZfo), (Xm,Cc,YZfp),(Xm,Cc,YZfq),(Xm,Cc,YZfr),(Xm,Cc,YZfs),(Xm,Cc,YZft),(Xm, Cc,YZfu),(Xm,Cc,YZfv),(Xm,Cc,YZfw),(Xm,Cc,YZfx),(Xm,Cc,YZfy),(Xm,Cc,YZfz), (Xm,Cc,YZga),(Xm,Cc,YZgb),(Xm,Cc,YZgc),(Xm,Cc,YZgd),(Xm,Cc,YZge),(Xm,Cc, YZgf),(Xm,Cc,YZgg),(Xm,Cc,YZgh),(Xm,Cc,YZgi),(Xm,Cc,YZgj),(Xm,Cc,YZgk), (Xm,Cc,YZgl),(Xm,Cc,YZgm),(Xm,Cc,YZgn),(Xm,Cc,YZgo),(Xm,Cc,YZgp),(Xm,Cc, YZgq),(Xm,Cc,YZgr),(Xm,Cc,YZgs),(Xm,Cc,YZgt),(Xm,Cd,YZa),(Xm,Cd,YZb),(Xm, Cd,YZc),(Xm,Cd,YZd),(Xm,Cd,YZe),(Xm,Cd,YZf),(Xm,Cd,YZg),(Xm,Cd,YZh), (Xm,Cd,YZi),(Xm,Cd,YZj),(Xm,Cd,YZk),(Xm,Cd,YZl),(Xm,Cd,YZm),(Xm,Cd,YZn), (Xm,Cd,YZo),(Xm,Cd,YZp),(Xm,Cd,YZq),(Xm,Cd,YZr),(Xm,Cd,YZs),(Xm,Cd,YZt), (Xm,Cd,YZu),(Xm,Cd,YZv),(Xm,Cd,YZw),(Xm,Cd,YZx),(Xm,Cd,YZy),(Xm,Cd,YZz), (Xm,Cd,YZaa),(Xm,Cd,YZab),(Xm,Cd,YZac),(Xm,Cd,YZad),(Xm,Cd,YZae),(Xm, Cd,YZaf),(Xm,Cd,YZag),(Xm,Cd,YZah),(Xm,Cd,YZai),(Xm,Cd,YZaj),(Xm,Cd,YZak), (Xm,Cd,YZal),(Xm,Cd,YZam),(Xm,Cd,YZan),(Xm,Cd,YZao),(Xm,Cd,YZap),(Xm, Cd,YZaq),(Xm,Cd,YZar),(Xm,Cd,YZas),(Xm,Cd,YZat),(Xm,Cd,YZau),(Xm,Cd,YZav), (Xm,Cd,YZaw),(Xm,Cd,YZax),(Xm,Cd,YZay),(Xm,Cd,YZaz),(Xm,Cd,YZba),(Xm, Cd,YZbb),(Xm,Cd,YZbc),(Xm,Cd,YZbd),(Xm,Cd,YZbe),(Xm,Cd,YZbf),(Xm,Cd, YZbg),(Xm,Cd,YZbh),(Xm,Cd,YZbi),(Xm,Cd,YZbj),(Xm,Cd,YZbk),(Xm,Cd,YZbl),(Xm, Cd,YZbm),(Xm,Cd,YZbn),(Xm,Cd,YZbo),(Xm,Cd,YZbp),(Xm,Cd,YZbq),(Xm,Cd, YZbr),(Xm,Cd,YZbs),(Xm,Cd,YZbt),(Xm,Cd,YZbu),(Xm,Cd,YZbv),(Xm,Cd,YZbw), (Xm,Cd,YZbx),(Xm,Cd,YZby),(Xm,Cd,YZbz),(Xm,Cd,YZca),(Xm,Cd,YZcb),(Xm, Cd,YZcc),(Xm,Cd,YZcd),(Xm,Cd,YZce),(Xm,Cd,YZcf),(Xm,Cd,YZcg),(Xm,Cd,YZch), (Xm,Cd,YZci),(Xm,Cd,YZcj),(Xm,Cd,YZck),(Xm,Cd,YZcl),(Xm,Cd,YZcm),(Xm, Cd,YZcn),(Xm,Cd,YZco),(Xm,Cd,YZcp),(Xm,Cd,YZcq),(Xm,Cd,YZcr),(Xm,Cd,YZcs), (Xm,Cd,YZct),(Xm,Cd,YZcu),(Xm,Cd,YZcv),(Xm,Cd,YZcw),(Xm,Cd,YZcx),(Xm, Cd,YZcy),(Xm,Cd,YZcz),(Xm,Cd,YZda),(Xm,Cd,YZdb),(Xm,Cd,YZdc),(Xm,Cd,YZdd), (Xm,Cd,YZde),(Xm,Cd,YZdf),(Xm,Cd,YZdg),(Xm,Cd,YZdh),(Xm,Cd,YZdi),(Xm, Cd,YZdj),(Xm,Cd,YZdk),(Xm,Cd,YZdl),(Xm,Cd,YZdm),(Xm,Cd,YZdn),(Xm,Cd, YZdo),(Xm,Cd,YZdp),(Xm,Cd,YZdq),(Xm,Cd,YZdr),(Xm,Cd,YZds),(Xm,Cd,YZdt),(Xm, Cd,YZdu),(Xm,Cd,YZdv),(Xm,Cd,YZdw),(Xm,Cd,YZdx),(Xm,Cd,YZdy),(Xm,Cd, YZdz),(Xm,Cd,YZea),(Xm,Cd,YZeb),(Xm,Cd,YZec),(Xmn,Cd,YZed),(Xm,Cd,YZee), (Xm,Cd,YZef),(Xm,Cd,YZeg),(Xm,Cd,YZeh),(Xm,Cd,YZei),(Xm,Cd,YZej),(Xm,Cd, YZek),(Xm,Cd,YZel),(Xm,Cd,YZem),(Xm,Cd,YZen),(Xm,Cd,YZeo),(Xm,Cd,YZep), (Xm,Cd,YZeq),(Xm,Cd,YZer),(Xm,Cd,YZes),(Xm,Cd,YZet),(Xm,Cd,YZeu),(Xm,Cd, YZev),(Xm,Cd,YZew),(Xm,Cd,YZex),(Xm,Cd,YZey),(Xm,Cd,YZez),(Xm,Cd,YZfa),(Xm, Cd,YZfb),(Xm,Cd,YZfc),(Xm,Cd,YZfd),(Xm,Cd,YZfe),(Xm,Cd,YZff),(Xm,Cd,YZfg), (Xm,Cd,YZfh),(Xm,Cd,YZfi),(Xm,Cd,YZfj),(Xm,Cd,YZfk),(Xm,Cd,YZfl),(Xm, Cd,YZfm),(Xm,Cd,YZfn),(Xm,Cd,YZfo),(Xm,Cd,YZfp),(Xm,Cd,YZfq),(Xm,Cd,YZfr), (Xm,Cd,YZfs),(Xm,Cd,YZft),(Xm,Cd,YZfu),(Xm,Cd,YZfv),(Xm,Cd,YZfw),(Xm,Cd, YZfx),(Xm,Cd,YZfy),(Xm,Cd,YZfz),(Xm,Cd,YZga),(Xm,Cd,YZgb),(Xm,Cd,YZgc),(Xm, Cd,YZgd),(Xm,Cd,YZge),(Xm,Cd,YZgf),(Xm,Cd,YZgg),(Xm,Cd,YZgh),(Xm,Cd, YZgi),(Xm,Cd,YZgj),(Xm,Cd,YZgk),(Xm,Cd,YZgl),(Xm,Cd,YZgm),(Xm,Cd,YZgn), (Xm,Cd,YZgo),(Xm,Cd,YZgp),(Xm,Cd,YZgq),(Xm,Cd,YZgr),(Xm,Cd,YZgs),(Xm,Cd, YZgt),(Xm,Ce,YZa),(Xm,Ce,YZb),(Xm,Ce,YZc),(Xm,Ce,YZd),(Xm,Ce,YZe),(Xm, Ce,YZf),(Xm,Ce,YZg),(Xm,Ce,YZh),(Xm,Ce,YZi),(Xm,Ce,YZj),(Xm,Ce,YZk),(Xm, Ce,YZl),(Xm,Ce,YZm),(Xm,Ce,YZn),(Xm,Ce,YZo),(Xm,Ce,YZp),(Xm,Ce,YZq),(Xm, Ce,YZr),(Xm,Ce,YZs),(Xm,Ce,YZt),(Xm,Ce,YZu),(Xm,Ce,YZv),(Xm,Ce,YZw),(Xm, Ce,YZx),(Xm,Ce,YZy),(Xm,Ce,YZz),(Xm,Ce,YZaa),(Xm,Ce,YZab),(Xm,Ce,YZac),(Xm, Ce,YZad),(Xm,Ce,YZae),(Xm,Ce,YZaf),(Xm,Ce,YZag),(Xm,Ce,YZah),(Xm,Ce, YZai),(Xm,Ce,YZaj),(Xm,Ce,YZak),(Xm,Ce,YZal),(Xm,Ce,YZam),(Xm,Ce,YZan),(Xm, Ce,YZao),(Xm,Ce,YZap),(Xm,Ce,YZaq),(Xm,Ce,YZar),(Xm,Ce,YZas),(Xm,Ce, YZat),(Xm,Ce,YZau),(Xm,Ce,YZav),(Xm,Ce,YZaw),(Xm,Ce,YZax),(Xm,Ce,YZay),(Xm, Ce,YZaz),(Xm,Ce,YZba),(Xm,Ce,YZbb),(Xm,Ce,YZbc),(Xm,Ce,YZbd),(Xm,Ce, YZbe),(Xm,Ce,YZbf),(Xm,Ce,YZbg),(Xm,Ce,YZbh),(Xm,Ce,YZbi),(Xm,Ce,YZbj),(Xm, Ce,YZbk),(Xm,Ce,YZbl),(Xm,Ce,YZbm),(Xm,Ce,YZbn),(Xm,Ce,YZbo),(Xm,Ce, YZbp),(Xm,Ce,YZbq),(Xm,Ce,YZbr),(Xm,Ce,YZbs),(Xm,Ce,YZbt),(Xm,Ce,YZbu),(Xm, Ce,YZbv),(Xm,Ce,YZbw),(Xm,Ce,YZbx),(Xm,Ce,YZby),(Xm,Ce,YZbz),(Xm,Ce, YZca),(Xm,Ce,YZcb),(Xm,Ce,YZcc),(Xm,Ce,YZcd),(Xm,Ce,YZce),(Xm,Ce,YZcf),(Xm, Ce,YZcg),(Xm,Ce,YZch),(Xm,Ce,YZci),(Xm,Ce,YZcj),(Xm,Ce,YZck),(Xm,Ce,YZcl), (Xm,Ce,YZcm),(Xm,Ce,YZcn),(Xm,Ce,YZco),(Xm,Ce,YZcp),(Xm,Ce,YZcq),(Xm, Ce,YZcr),(Xm,Ce,YZcs),(Xm,Ce,YZct),(Xm,Ce,YZcu),(Xm,Ce,YZcv),(Xm,Ce,YZcw), (Xm,Ce,YZcx),(Xm,Ce,YZcy),(Xm,Ce,YZcz),(Xm,Ce,YZda),(Xm,Ce,YZdb),(Xm, Ce,YZdc),(Xm,Ce,YZdd),(Xm,Ce,YZde),(Xm,Ce,YZdf),(Xm,Ce,YZdg),(Xm,Ce,YZdh), (Xm,Ce,YZdi),(Xm,Ce,YZdj),(Xm,Ce,YZdk),(Xm,Ce,YZdl),(Xm,Ce,YZdm),(Xm, Ce,YZdn),(Xm,Ce,YZdo),(Xm,Ce,YZdp),(Xm,Ce,YZdq),(Xm,Ce,YZdr),(Xm,Ce,YZds), (Xm,Ce,YZdt),(Xm,Ce,YZdu),(Xm,Ce,YZdv),(Xm,Ce,YZdw),(Xm,Ce,YZdx),(Xm, Ce,YZdy),(Xm,Ce,YZdz),(Xm,Ce,YZea),(Xm,Ce,YZeb),(Xm,Ce,YZec),(Xm,Ce,YZed), (Xm,Ce,YZee),(Xm,Ce,YZef),(Xm,Ce,YZeg),(Xm,Ce,YZeh),(Xm,Ce,YZei),(Xm, Ce,YZej),(Xm,Ce,YZek),(Xm,Ce,YZel),(Xm,Ce,YZem),(Xm,Ce,YZen),(Xm,Ce,YZeo), (Xm,Ce,YZep),(Xm,Ce,YZeq),(Xm,Ce,YZer),(Xm,Ce,YZes),(Xm,Ce,YZet),(Xm, Ce,YZeu),(Xm,Ce,YZev),(Xm,Ce,YZew),(Xm,Ce,YZex),(Xm,Ce,YZey),(Xm,Ce,YZez), (Xm,Ce,YZfa),(Xm,Ce,YZfb),(Xm,Ce,YZfc),(Xm,Ce,YZfd),(Xm,Ce,YZfe),(Xm, Ce,YZff),(Xm,Ce,YZfg),(Xm,Ce,YZfh),(Xm,Ce,YZfi),(Xm,Ce,YZfj),(Xm,Ce,YZfk),(Xm, Ce,YZfl),(Xm,Ce,YZfm),(Xm,Ce,YZfn),(Xm,Ce,YZfo),(Xm,Ce,YZfp),(Xm,Ce,YZfq), (Xm,Ce,YZfr),(Xm,Ce,YZfs),(Xm,Ce,YZft),(Xm,Ce,YZfu),(Xm,Ce,YZfv),(Xm, Ce,YZfw),(Xm,Ce,YZfx),(Xm,Ce,YZfy),(Xm,Ce,YZfz),(Xm,Ce,YZga),(Xm,Ce,YZgb), (Xm,Ce,YZgc),(Xm,Ce,YZgd),(Xm,Ce,YZge),(Xm,Ce,YZgf),(Xm,Ce,YZgg),(Xm,Ce, YZgh),(Xm,Ce,YZgi),(Xm,Ce,YZgj),(Xm,Ce,YZgk),(Xm,Ce,YZgl),(Xm,Ce,YZgm), (Xm,Ce,YZgn),(Xm,Ce,YZgo),(Xm,Ce,YZgp),(Xm,Ce,YZgq),(Xm,Ce,YZgr),(Xm,Ce, YZgs),(Xmn,Ce,YZgt),(Xm,Cf,YZa),(Xm,Cf,YZb),(Xm,Cf,YZc),(Xm,Cf,YZd),(Xm, Cf,YZe),(Xm,Cf,YZf),(Xm,Cf,YZg),(Xm,Cf,YZh),(Xm,Cf,YZi),(Xm,Cf,YZj),(Xm,Cf, YZk),(Xm,Cf,YZl),(Xm,Cf,YZm),(Xm,Cf,YZn),(Xm,Cf,YZo),(Xm,Cf,YZp),(Xm,Cf, YZq),(Xm,Cf,YZr),(Xm,Cf,YZs),(Xm,Cf,YZt),(Xm,Cf,YZu),(Xm,Cf,YZv),(Xm,Cf,YZw), (Xm,Cf,YZx),(Xm,Cf,YZy),(Xm,Cf,YZz),(Xm,Cf,YZaa),(Xm,Cf,YZab),(Xm,Cf, YZac),(Xm,Cf,YZad),(Xm,Cf,YZae),(Xm,Cf,YZaf),(Xm,Cf,YZag),(Xm,Cf,YZah),(Xm, Cf,YZai),(Xm,Cf,YZaj),(Xm,Cf,YZak),(Xm,Cf,YZal),(Xm,Cf,YZam),(Xm,Cf,YZan), (Xm,Cf,YZao),(Xm,Cf,YZap),(Xm,Cf,YZaq),(Xm,Cf,YZar),(Xm,Cf,YZas),(Xm,Cf, YZat),(Xm,Cf,YZau),(Xm,Cf,YZav),(Xm,Cf,YZaw),(Xm,Cf,YZax),(Xm,Cf,YZay),(Xm, Cf,YZaz),(Xm,Cf,YZba),(Xm,Cf,YZbb),(Xm,Cf,YZbc),(Xm,Cf,YZbd),(Xm,Cf,YZbe), (Xm,Cf,YZbf),(Xm,Cf,YZbg),(Xm,Cf,YZbh),(Xm,Cf,YZbi),(Xm,Cf,YZbj),(Xm, Cf,YZbk),(Xm,Cf,YZbl),(Xm,Cf,YZbm),(Xm,Cf,YZbn),(Xm,Cf,YZbo),(Xm,Cf,YZbp), (Xm,Cf,YZbq),(Xm,Cf,YZbr),(Xm,Cf,YZbs),(Xm,Cf,YZbt),(Xm,Cf,YZbu),(Xm,Cf, YZbv),(Xm,Cf,YZbw),(Xm,Cf,YZbx),(Xm,Cf,YZby),(Xm,Cf,YZbz),(Xm,Cf,YZca),(Xm, Cf,YZcb),(Xm,Cf,YZcc),(Xm,Cf,YZcd),(Xm,Cf,YZce),(Xm,Cf,YZcf),(Xm,Cf,YZcg), (Xm,Cf,YZch),(Xm,Cf,YZci),(Xm,Cf,YZcj),(Xm,Cf,YZck),(Xm,Cf,YZcl),(Xm, Cf,YZcm),(Xm,Cf,YZcn),(Xm,Cf,YZco),(Xm,Cf,YZcp),(Xm,Cf,YZcq),(Xm,Cf,YZcr), (Xm,Cf,YZcs),(Xm,Cf,YZct),(Xm,Cf,YZcu),(Xm,Cf,YZcv),(Xm,Cf,YZcw),(Xm,Cf, YZcx),(Xm,Cf,YZcy),(Xm,Cf,YZcz),(Xm,Cf,YZda),(Xm,Cf,YZdb),(Xm,Cf,YZdc),(Xm, Cf,YZdd),(Xm,Cf,YZde),(Xm,Cf,YZdf),(Xm,Cf,YZdg),(Xm,Cf,YZdh),(Xm,Cf,YZdi), (Xm,Cf,YZdj),(Xm,Cf,YZdk),(Xm,Cf,YZdl),(Xm,Cf,YZdm),(Xm,Cf,YZdn),(Xm, Cf,YZdo),(Xm,Cf,YZdp),(Xm,Cf,YZdq),(Xm,Cf,YZdr),(Xm,Cf,YZds),(Xm,Cf,YZdt), (Xm,Cf,YZdu),(Xm,Cf,YZdv),(Xm,Cf,YZdw),(Xm,Cf,YZdx),(Xm,Cf,YZdy),(Xm,Cf, YZdz),(Xm,Cf,YZea),(Xm,Cf,YZeb),(Xm,Cf,YZec),(Xm,Cf,YZed),(Xm,Cf,YZee),(Xm, Cf,YZef),(Xm,Cf,YZeg),(Xm,Cf,YZeh),(Xm,Cf,YZei),(Xm,Cf,YZej),(Xm,Cf,YZek), (Xm,Cf,YZel),(Xm,Cf,YZem),(Xm,Cf,YZen),(Xm,Cf,YZeo),(Xm,Cf,YZep),(Xm,Cf, YZeq),(Xm,Cf,YZer),(Xm,Cf,YZes),(Xm,Cf,YZet),(Xm,Cf,YZeu),(Xm,Cf,YZev),(Xm, Cf,YZew),(Xm,Cf,YZex),(Xm,Cf,YZey),(Xm,Cf,YZez),(Xm,Cf,YZfa),(Xm,Cf,YZfb), (Xm,Cf,YZfc),(Xm,Cf,YZfd),(Xm,Cf,YZfe),(Xm,Cf,YZff),(Xm,Cf,YZfg),(Xm,Cf, YZfh),(Xm,Cf,YZfi),(Xm,Cf,YZfj),(Xm,Cf,YZfk),(Xm,Cf,YZfl),(Xm,Cf,YZfm),(Xm, Cf,YZfn),(Xm,Cf,YZfo),(Xm,Cf,YZfp),(Xm,Cf,YZfq),(Xm,Cf,YZfr),(Xm,Cf,YZfs),(Xm, Cf,YZft),(Xm,Cf,YZfu),(Xm,Cf,YZfv),(Xm,Cf,YZfw),(Xm,Cf,YZfx),(Xm,Cf,YZfy), (Xm,Cf,YZfz),(Xm,Cf,YZga),(Xm,Cf,YZgb),(Xm,Cf,YZgc),(Xm,Cf,YZgd),(Xm, Cf,YZge),(Xm,Cf,YZgf),(Xm,Cf,YZgg),(Xm,Cf,YZgh),(Xm,Cf,YZgi),(Xm,Cf,YZgj), (Xm,Cf,YZgk),(Xm,Cf,YZgl),(Xm,Cf,YZgm),(Xm,Cf,YZgn),(Xm,Cf,YZgo),(Xm,Cf, YZgp),(Xm,Cf,YZgq),(Xm,Cf,YZgr),(Xm,Cf,YZgs),(Xm,Cf,YZgt), (Xn,Ca,YZa),(Xn,Ca,YZb),(Xn,Ca,YZc),(Xn,Ca,YZd),(Xn,Ca,YZe),(Xn,Ca,YZf),(Xn, Ca,YZg),(Xn,Ca,YZh),(Xn,Ca,YZi),(Xn,Ca,YZj),(Xn,Ca,YZk),(Xn,Ca,YZl),(Xn,Ca,YZm), (Xn,Ca,YZn),(Xn,Ca,YZo),(Xn,Ca,YZp),(Xn,Ca,YZq),(Xn,Ca,YZr),(Xn,Ca,YZs), (Xn,Ca,YZt),(Xn,Ca,YZu),(Xn,Ca,YZv),(Xn,Ca,YZw),(Xn,Ca,YZx),(Xn,Ca,YZy),(Xn, Ca,YZz),(Xn,Ca,YZaa),(Xn,Ca,YZab),(Xn,Ca,YZac),(Xn,Ca,YZad),(Xn,Ca,YZae),(Xn, Ca,YZaf),(Xn,Ca,YZag),(Xn,Ca,YZah),(Xn,Ca,YZai),(Xn,Ca,YZaj),(Xn,Ca,YZak), (Xn,Ca,YZal),(Xn,Ca,YZam),(Xn,Ca,YZan),(Xn,Ca,YZao),(Xn,Ca,YZap),(Xn,Ca,YZaq), (Xn,Ca,YZar),(Xn,Ca,YZas),(Xn,Ca,YZat),(Xn,Ca,YZau),(Xn,Ca,YZav),(Xn,Ca, YZaw),(Xn,Ca,YZax),(Xn,Ca,YZay),(Xn,Ca,YZaz),(Xn,Ca,YZba),(Xn,Ca,YZbb),(Xn, Ca,YZbc),(Xn,Ca,YZbd),(Xn,Ca,YZbe),(Xn,Ca,YZbf),(Xn,Ca,YZbg),(Xn,Ca,YZbh),(Xn, Ca,YZbi),(Xn,Ca,YZbj),(Xn,Ca,YZbk),(Xn,Ca,YZbl),(Xn,Ca,YZbm),(Xn,Ca,YZbn), (Xn,Ca,YZbo),(Xn,Ca,YZbp),(Xn,Ca,YZbq),(Xn,Ca,YZbr),(Xn,Ca,YZbs),(Xn,Ca, YZbt),(Xn,Ca,YZbu),(Xn,Ca,YZbv),(Xn,Ca,YZbw),(Xn,Ca,YZbx),(Xn,Ca,YZby),(Xn, Ca,YZbz),(Xn,Ca,YZca),(Xn,Ca,YZcb),(Xn,Ca,YZcc),(Xn,Ca,YZcd),(Xn,Ca,YZce),(Xn, Ca,YZcf),(Xn,Ca,YZcg),(Xn,Ca,YZch),(Xn,Ca,YZci),(Xn,Ca,YZcj),(Xn,Ca,YZck), (Xn,Ca,YZcl),(Xn,Ca,YZcm),(Xn,Ca,YZcn),(Xn,Ca,YZco),(Xn,Ca,YZcp),(Xn,Ca,YZcq), (Xn,Ca,YZcr),(Xn,Ca,YZcs),(Xn,Ca,YZct),(Xn,Ca,YZcu),(Xn,Ca,YZcv),(Xn,Ca, YZcw),(Xn,Ca,YZcx),(Xn,Ca,YZcy),(Xn,Ca,YZcz),(Xn,Ca,YZda),(Xn,Ca,YZdb),(Xn, Ca,YZdc),(Xn,Ca,YZdd),(Xn,Ca,YZde),(Xn,Ca,YZdf),(Xn,Ca,YZdg),(Xn,Ca,YZdh),(Xn, Ca,YZdi),(Xn,Ca,YZdj),(Xn,Ca,YZdk),(Xn,Ca,YZdl),(Xn,Ca,YZdm),(Xn,Ca,YZdn), (Xn,Ca,YZdo),(Xn,Ca,YZdp),(Xn,Ca,YZdq),(Xn,Ca,YZdr),(Xn,Ca,YZds),(Xn,Ca, YZdt),(Xn,Ca,YZdu),(Xn,Ca,YZdv),(Xn,Ca,YZdw),(Xn,Ca,YZdx),(Xn,Ca,YZdy),(Xn, Ca,YZdz),(Xn,Ca,YZea),(Xn,Ca,YZeb),(Xn,Ca,YZec),(Xn,Ca,YZed),(Xn,Ca,YZee),(Xn, Ca,YZef),(Xn,Ca,YZeg),(Xn,Ca,YZeh),(Xn,Ca,YZei),(Xn,Ca,YZej),(Xn,Ca,YZek), (Xn,Ca,YZel),(Xn,Ca,YZem),(Xn,Ca,YZen),(Xn,Ca,YZeo),(Xn,Ca,YZep),(Xn,Ca,YZeq), (Xn,Ca,YZer),(Xn,Ca,YZes),(Xn,Ca,YZet),(Xn,Ca,YZeu),(Xn,Ca,YZev),(Xn,Ca, YZew),(Xn,Ca,YZex),(Xn,Ca,YZey),(Xn,Ca,YZez),(Xn,Ca,YZfa),(Xn,Ca,YZfb),(Xn, Ca,YZfc),(Xn,Ca,YZfd),(Xn,Ca,YZfe),(Xn,Ca,YZff),(Xn,Ca,YZfg),(Xn,Ca,YZfh),(Xn, Ca,YZfi),(Xn,Ca,YZfj),(Xn,Ca,YZfk),(Xn,Ca,YZfl),(Xn,Ca,YZfm),(Xn,Ca,YZfn),(Xn, Ca,YZfo),(Xn,Ca,YZfp),(Xn,Ca,YZfq),(Xn,Ca,YZfr),(Xn,Ca,YZfs),(Xn,Ca,YZft),(Xn, Ca,YZfu),(Xn,Ca,YZfv),(Xn,Ca,YZfw),(Xn,Ca,YZfx),(Xn,Ca,YZfy),(Xn,Ca,YZfz),(Xn, Ca,YZga),(Xn,Ca,YZgb),(Xn,Ca,YZgc),(Xn,Ca,YZgd),(Xn,Ca,YZge),(Xn,Ca,YZgf), (Xn,Ca,YZgg),(Xn,Ca,YZgh),(Xn,Ca,YZgi),(Xn,Ca,YZgj),(Xn,Ca,YZgk),(Xn,Ca,YZgl), (Xn,Ca,YZgm),(Xn,Ca,YZgn),(Xn,Ca,YZgo),(Xn,Ca,YZgp),(Xn,Ca,YZgq),(Xn, Ca,YZgr),(Xn,Ca,YZgs),(Xn,Ca,YZgt),(Xn,Cb,YZa),(Xn,Cb,YZb),(Xn,Cb,YZc),(Xn, Cb,YZd),(Xn,Cb,YZe),(Xn,Cb,YZf),(Xn,Cb,YZg),(Xn,Cb,YZh),(Xn,Cb,YZi),(Xn,Cb, YZj),(Xn,Cb,YZk),(Xn,Cb,YZl),(Xn,Cb,YZm),(Xn,Cb,YZn),(Xn,Cb,YZo),(Xn,Cb,YZp), (Xn,Cb,YZq),(Xn,Cb,YZr),(Xn,Cb,YZs),(Xn,Cb,YZt),(Xn,Cb,YZu),(Xn,Cb,YZv),(Xn, Cb,YZw),(Xn,Cb,YZx),(Xn,Cb,YZy),(Xn,Cb,YZz),(Xn,Cb,YZaa),(Xn,Cb,YZab),(Xn, Cb,YZac),(Xn,Cb,YZad),(Xn,Cb,YZae),(Xn,Cb,YZaf),(Xn,Cb,YZag),(Xn,Cb,YZah), (Xn,Cb,YZai),(Xn,Cb,YZaj),(Xn,Cb,YZak),(Xn,Cb,YZal),(Xn,Cb,YZam),(Xn,Cb, YZan),(Xn,Cb,YZao),(Xn,Cb,YZap),(Xn,Cb,YZaq),(Xn,Cb,YZar),(Xn,Cb,YZas),(Xn, Cb,YZat),(Xn,Cb,YZau),(Xn,Cb,YZav),(Xn,Cb,YZaw),(Xn,Cb,YZax),(Xn,Cb,YZay), (Xn,Cb,YZaz),(Xn,Cb,YZba),(Xn,Cb,YZbb),(Xn,Cb,YZbc),(Xn,Cb,YZbd),(Xn,Cb,YZbe), (Xn,Cb,YZbf),(Xn,Cb,YZbg),(Xn,Cb,YZbh),(Xn,Cb,YZbi),(Xn,Cb,YZbj),(Xn,Cb, YZbk),(Xn,Cb,YZbl),(Xn,Cb,YZbm),(Xn,Cb,YZbn),(Xn,Cb,YZbo),(Xn,Cb,YZbp),(Xn, Cb,YZbq),(Xn,Cb,YZbr),(Xn,Cb,YZbs),(Xn,Cb,YZbt),(Xn,Cb,YZbu),(Xn,Cb,YZbv), (Xn,Cb,YZbw),(Xn,Cb,YZbx),(Xn,Cb,YZby),(Xn,Cb,YZbz),(Xn,Cb,YZca),(Xn,Cb, YZcb),(Xn,Cb,YZcc),(Xn,Cb,YZcd),(Xn,Cb,YZce),(Xn,Cb,YZcf),(Xn,Cb,YZcg),(Xn, Cb,YZch),(Xn,Cb,YZci),(Xn,Cb,YZcj),(Xn,Cb,YZck),(Xn,Cb,YZcl),(Xn,Cb,YZcm),(Xn, Cb,YZcn),(Xn,Cb,YZco),(Xn,Cb,YZcp),(Xn,Cb,YZcq),(Xn,Cb,YZcr),(Xn,Cb,YZcs), (Xn,Cb,YZct),(Xn,Cb,YZcu),(Xn,Cb,YZcv),(Xn,Cb,YZcw),(Xn,Cb,YZcx),(Xn,Cb, YZcy),(Xn,Cb,YZcz),(Xn,Cb,YZda),(Xn,Cb,YZdb),(Xn,Cb,YZdc),(Xn,Cb,YZdd),(Xn, Cb,YZde),(Xn,Cb,YZdf),(Xn,Cb,YZdg),(Xn,Cb,YZdh),(Xn,Cb,YZdi),(Xn,Cb,YZdj),(Xn, Cb,YZdk),(Xn,Cb,YZdl),(Xn,Cb,YZdm),(Xn,Cb,YZdn),(Xn,Cb,YZdo),(Xn,Cb,YZdp), (Xn,Cb,YZdq),(Xn,Cb,YZdr),(Xn,Cb,YZds),(Xn,Cb,YZdt),(Xn,Cb,YZdu),(Xn,Cb, YZdv),(Xn,Cb,YZdw),(Xn,Cb,YZdx),(Xn,Cb,YZdy),(Xn,Cb,YZdz),(Xn,Cb,YZea),(Xn, Cb,YZeb),(Xn,Cb,YZec),(Xn,Cb,YZed),(Xn,Cb,YZee),(Xn,Cb,YZef),(Xn,Cb,YZeg), (Xn,Cb,YZeh),(Xn,Cb,YZei),(Xn,Cb,YZej),(Xn,Cb,YZek),(Xn,Cb,YZel),(Xn,Cb,YZem), (Xn,Cb,YZen),(Xn,Cb,YZeo),(Xn,Cb,YZep),(Xn,Cb,YZeq),(Xn,Cb,YZer),(Xn,Cb, YZes),(Xn,Cb,YZet),(Xn,Cb,YZeu),(Xn,Cb,YZev),(Xn,Cb,YZew),(Xn,Cb,YZex),(Xn, Cb,YZey),(Xn,Cb,YZez),(Xn,Cb,YZfa),(Xn,Cb,YZfb),(Xn,Cb,YZfc),(Xn,Cb,YZfd),(Xn, Cb,YZfe),(Xn,Cb,YZff),(Xn,Cb,YZfg),(Xn,Cb,YZfh),(Xn,Cb,YZfi),(Xn,Cb,YZfj), (Xn,Cb,YZfk),(Xn,Cb,YZfl),(Xn,Cb,YZfm),(Xn,Cb,YZfn),(Xn,Cb,YZfo),(Xn,Cb,YZfp), (Xn,Cb,YZfq),(Xn,Cb,YZfr),(Xn,Cb,YZfs),(Xn,Cb,YZft),(Xn,Cb,YZfu),(Xn,Cb,YZfv), (Xn,Cb,YZfw),(Xn,Cb,YZfx),(Xn,Cb,YZfy),(Xn,Cb,YZfz),(Xn,Cb,YZga),(Xn,Cb, YZgb),(Xn,Cb,YZgc),(Xn,Cb,YZgd),(Xn,Cb,YZge),(Xn,Cb,YZgf),(Xn,Cb,YZgg),(Xn, Cb,YZgh),(Xn,Cb,YZgi),(Xn,Cb,YZgj),(Xn,Cb,YZgk),(Xn,Cb,YZgl),(Xn,Cb,YZgm), (Xn,Cb,YZgn),(Xn,Cb,YZgo),(Xn,Cb,YZgp),(Xn,Cb,YZgq),(Xn,Cb,YZgr),(Xn,Cb,YZgs), (Xn,Cb,YZgt),(Xn,Cc,YZa),(Xn,Cc,YZb),(Xn,Cc,YZc),(Xn,Cc,YZd),(Xn,Cc,YZe), (Xn,Cc,YZf),(Xn,Cc,YZg),(Xn,Cc,YZh),(Xn,Cc,YZi),(Xn,Cc,YZj),(Xn,Cc,YZk),(Xn, Cc,YZl),(Xn,Cc,YZm),(Xn,Cc,YZn),(Xn,Cc,YZo),(Xn,Cc,YZp),(Xn,Cc,YZq),(Xn,Cc, YZr),(Xn,Cc,YZs),(Xn,Cc,YZt),(Xn,Cc,YZu),(Xn,Cc,YZv),(Xn,Cc,YZw),(Xn,Cc,YZx), (Xn,Cc,YZy),(Xn,Cc,YZz),(Xn,Cc,YZaa),(Xn,Cc,YZab),(Xn,Cc,YZac),(Xn,Cc,YZad), (Xn,Cc,YZae),(Xn,Cc,YZaf),(Xn,Cc,YZag),(Xn,Cc,YZah),(Xn,Cc,YZai),(Xn,Cc, YZaj),(Xn,Cc,YZak),(Xn,Cc,YZal),(Xn,Cc,YZam),(Xn,Cc,YZan),(Xn,Cc,YZao),(Xn, Cc,YZap),(Xn,Cc,YZaq),(Xn,Cc,YZar),(Xn,Cc,YZas),(Xn,Cc,YZat),(Xn,Cc,YZau),(Xn, Cc,YZav),(Xn,Cc,YZaw),(Xn,Cc,YZax),(Xn,Cc,YZay),(Xn,Cc,YZaz),(Xn,Cc,YZba), (Xn,Cc,YZbb),(Xn,Cc,YZbc),(Xn,Cc,YZbd),(Xn,Cc,YZbe),(Xn,Cc,YZbf),(Xn,Cc,YZbg), (Xn,Cc,YZbh),(Xn,Cc,YZbi),(Xn,Cc,YZbj),(Xn,Cc,YZbk),(Xn,Cc,YZbl),(Xn,Cc, YZbm),(Xn,Cc,YZbn),(Xn,Cc,YZbo),(Xn,Cc,YZbp),(Xn,Cc,YZbq),(Xn,Cc,YZbr),(Xn, Cc,YZbs),(Xn,Cc,YZbt),(Xn,Cc,YZbu),(Xn,Cc,YZbv),(Xn,Cc,YZbw),(Xn,Cc,YZbx), (Xn,Cc,YZby),(Xn,Cc,YZbz),(Xn,Cc,YZca),(Xn,Cc,YZcb),(Xn,Cc,YZcc),(Xn,Cc,YZcd), (Xn,Cc,YZce),(Xn,Cc,YZcf),(Xn,Cc,YZcg),(Xn,Cc,YZch),(Xn,Cc,YZci),(Xn,Cc, YZcj),(Xn,Cc,YZck),(Xn,Cc,YZcl),(Xn,Cc,YZcm),(Xn,Cc,YZcn),(Xn,Cc,YZco),(Xn, Cc,YZcp),(Xn,Cc,YZcq),(Xn,Cc,YZcr),(Xn,Cc,YZcs),(Xn,Cc,YZct),(Xn,Cc,YZcu),(Xn, Cc,YZcv),(Xn,Cc,YZcw),(Xn,Cc,YZcx),(Xn,Cc,YZcy),(Xn,Cc,YZcz),(Xn,Cc,YZda), (Xn,Cc,YZdb),(Xn,Cc,YZdc),(Xn,Cc,YZdd),(Xn,Cc,YZde),(Xn,Cc,YZdf),(Xn,Cc,YZdg), (Xn,Cc,YZdh),(Xn,Cc,YZdi),(Xn,Cc,YZdj),(Xn,Cc,YZdk),(Xn,Cc,YZdl),(Xn,Cc, YZdm),(Xn,Cc,YZdn),(Xn,Cc,YZdo),(Xn,Cc,YZdp),(Xn,Cc,YZdq),(Xn,Cc,YZdr),(Xn, Cc,YZds),(Xn,Cc,YZdt),(Xn,Cc,YZdu),(Xn,Cc,YZdv),(Xn,Cc,YZdw),(Xn,Cc,YZdx), (Xn,Cc,YZdy),(Xn,Cc,YZdz),(Xn,Cc,YZea),(Xn,Cc,YZeb),(Xn,Cc,YZec),(Xn,Cc,YZed), (Xn,Cc,YZee),(Xn,Cc,YZef),(Xn,Cc,YZeg),(Xn,Cc,YZeh),(Xn,Cc,YZei),(Xn,Cc, YZej),(Xn,Cc,YZek),(Xn,Cc,YZel),(Xn,Cc,YZem),(Xn,Cc,YZen),(Xn,Cc,YZeo),(Xn, Cc,YZep),(Xn,Cc,YZeq),(Xn,Cc,YZer),(Xn,Cc,YZes),(Xn,Cc,YZet),(Xn,Cc,YZeu),(Xn, Cc,YZev),(Xn,Cc,YZew),(Xn,Cc,YZex),(Xn,Cc,YZey),(Xn,Cc,YZez),(Xn,Cc,YZfa), (Xn,Cc,YZfb),(Xn,Cc,YZfc),(Xn,Cc,YZfd),(Xn,Cc,YZfe),(Xn,Cc,YZff),(Xn,Cc,YZfg), (Xn,Cc,YZfh),(Xn,Cc,YZfi),(Xn,Cc,YZfj),(Xn,Cc,YZfk),(Xn,Cc,YZfl),(Xn,Cc,YZfm), (Xn,Cc,YZfn),(Xn,Cc,YZfo),(Xn,Cc,YZfp),(Xn,Cc,YZfq),(Xn,Cc,YZfr),(Xn,Cc,YZfs), (Xn,Cc,YZft),(Xn,Cc,YZfu),(Xn,Cc,YZfv),(Xn,Cc,YZfw),(Xn,Cc,YZfx),(Xn,Cc, YZfy),(XCn,Cc,YZfz),(Xn,Cc,YZga),(Xn,Cc,YZgb),(Xn,Cc,YZgc),(Xn,Cc,YZgd),(Xn, Cc,YZge),(Xn,Cc,YZgf),(Xn,Cc,YZgg),(Xn,Cc,YZgh),(Xn,Cc,YZgi),(Xn,Cc,YZgj),(Xn, Cc,YZgk),(Xn,Cc,YZgl),(Xn,Cc,YZgm),(Xn,Cc,YZgn),(Xn,Cc,YZgo),(Xn,Cc,YZgp), (Xn,Cc,YZgq),(Xn,Cc,YZgr),(Xn,Cc,YZgs),(Xn,Cc,YZgt),(Xn,Cd,YZa),(Xn,Cd,YZb), (Xn,Cd,YZc),(Xn,Cd,YZd),(Xn,Cd,YZe),(Xn,Cd,YZf),(Xn,Cd,YZg),(Xn,Cd,YZh), (Xn,Cd,YZi),(Xn,Cd,YZj),(Xn,Cd,YZk),(Xn,Cd,YZl),(Xn,Cd,YZm),(Xn,Cd,YZn),(Xn, Cd,YZo),(Xn,Cd,YZp),(Xn,Cd,YZq),(Xn,Cd,YZr),(Xn,Cd,YZs),(Xn,Cd,YZt),(Xn,Cd, YZu),(Xn,Cd,YZv),(Xn,Cd,YZw),(Xn,Cd,YZx),(Xn,Cd,YZy),(Xn,Cd,YZz),(Xn,Cd, YZaa),(Xn,Cd,YZab),(Xn,Cd,YZac),(Xn,Cd,YZad),(Xn,Cd,YZae),(Xn,Cd,YZaf),(Xn, Cd,YZag),(Xn,Cd,YZah),(Xn,Cd,YZai),(Xn,Cd,YZaj),(Xn,Cd,YZak),(Xn,Cd,YZal),(Xn, Cd,YZam),(Xn,Cd,YZan),(Xn,Cd,YZao),(Xn,Cd,YZap),(Xn,Cd,YZaq),(Xn,Cd,YZar), (Xn,Cd,YZas),(Xn,Cd,YZat),(Xn,Cd,YZau),(Xn,Cd,YZav),(Xn,Cd,YZaw),(Xn,Cd, YZax),(Xn,Cd,YZay),(Xn,Cd,YZaz),(Xn,Cd,YZba),(Xn,Cd,YZbb),(Xn,Cd,YZbc),(Xn, Cd,YZbd),(Xn,Cd,YZbe),(Xn,Cd,YZbf),(Xn,Cd,YZbg),(Xn,Cd,YZbh),(Xn,Cd,YZbi), (Xn,Cd,YZbj),(Xn,Cd,YZbk),(Xn,Cd,YZbl),(Xn,Cd,YZbm),(Xn,Cd,YZbn),(Xn,Cd,YZbo), (Xn,Cd,YZbp),(Xn,Cd,YZbq),(Xn,Cd,YZbr),(Xn,Cd,YZbs),(Xn,Cd,YZbt),(Xn,Cd, YZbu),(Xn,Cd,YZbv),(Xn,Cd,YZbw),(Xn,Cd,YZbx),(Xn,Cd,YZby),(Xn,Cd,YZbz),(Xn, Cd,YZca),(Xn,Cd,YZcb),(Xn,Cd,YZcc),(Xn,Cd,YZcd),(Xn,Cd,YZce),(Xn,Cd,YZcf), (Xn,Cd,YZcg),(Xn,Cd,YZch),(Xn,Cd,YZci),(Xn,Cd,YZcj),(Xn,Cd,YZck),(Xn,Cd,YZcl), (Xn,Cd,YZcm),(Xn,Cd,YZcn),(Xn,Cd,YZco),(Xn,Cd,YZcp),(Xn,Cd,YZcq),(Xn, Cd,YZcr),(Xn,Cd,YZcs),(Xn,Cd,YZct),(Xn,Cd,YZcu),(Xn,Cd,YZcv),(Xn,Cd,YZcw),(Xn, Cd,YZcx),(Xn,Cd,YZcy),(Xn,Cd,YZcz),(Xn,Cd,YZda),(Xn,Cd,YZdb),(Xn,Cd,YZdc), (Xn,Cd,YZdd),(Xn,Cd,YZde),(Xn,Cd,YZdf),(Xn,Cd,YZdg),(Xn,Cd,YZdh),(Xn,Cd, YZdi),(Xn,Cd,YZdj),(Xn,Cd,YZdk),(Xn,Cd,YZdl),(Xn,Cd,YZdm),(Xn,Cd,YZdn),(Xn, Cd,YZdo),(Xn,Cd,YZdp),(Xn,Cd,YZdq),(Xn,Cd,YZdr),(Xn,Cd,YZds),(Xn,Cd,YZdt), (Xn,Cd,YZdu),(Xn,Cd,YZdv),(Xn,Cd,YZdw),(Xn,Cd,YZdx),(Xn,Cd,YZdy),(Xn,Cd, YZdz),(Xn,Cd,YZea),(Xn,Cd,YZeb),(Xn,Cd,YZec),(Xn,Cd,YZed),(Xn,Cd,YZee),(Xn, Cd,YZef),(Xn,Cd,YZeg),(Xn,Cd,YZeh),(Xn,Cd,YZei),(Xn,Cd,YZej),(Xn,Cd,YZek),(Xn, Cd,YZel),(Xn,Cd,YZem),(Xn,Cd,YZen),(Xn,Cd,YZeo),(Xn,Cd,YZep),(Xn,Cd,YZeq), (Xn,Cd,YZer),(Xn,Cd,YZes),(Xn,Cd,YZet),(Xn,Cd,YZeu),(Xn,Cd,YZev),(Xn,Cd, YZew),(Xn,Cd,YZex),(Xn,Cd,YZey),(Xn,Cd,YZez),(Xn,Cd,YZfa),(Xn,Cd,YZfb),(Xn, Cd,YZfc),(Xn,Cd,YZfd),(Xn,Cd,YZfe),(Xn,Cd,YZff),(Xn,Cd,YZfg),(Xn,Cd,YZfh),(Xn, Cd,YZfi),(Xn,Cd,YZfj),(Xn,Cd,YZfk),(Xn,Cd,YZfl),(Xn,Cd,YZfm),(Xn,Cd,YZfn),(Xn, Cd,YZfo),(Xn,Cd,YZfp),(Xn,Cd,YZfq),(Xn,Cd,YZfr),(Xn,Cd,YZfs),(Xn,Cd,YZft), (Xn,Cd,YZfu),(Xn,Cd,YZfv),(Xn,Cd,YZfw),(Xn,Cd,YZfx),(Xn,Cd,YZfy),(Xn,Cd,YZfz), (Xn,Cd,YZga),(Xn,Cd,YZgb),(Xn,Cd,YZgc),(Xn,Cd,YZgd),(Xn,Cd,YZge),(Xn,Cd, YZgf),(Xn,Cd,YZgg),(Xn,Cd,YZgh),(Xn,Cd,YZgi),(Xn,Cd,YZgj),(Xn,Cd,YZgk),(Xn, Cd,YZgl),(Xn,Cd,YZgm),(Xn,Cd,YZgn),(Xn,Cd,YZgo),(Xn,Cd,YZgp),(Xn,Cd,YZgq), (Xn,Cd,YZgr),(Xn,Cd,YZgs),(Xn,Cd,YZgt),(Xn,Ce,YZa),(Xn,Ce,YZb),(Xn,Ce,YZc), (Xn,Ce,YZd),(Xn,Ce,YZe),(Xn,Ce,YZf),(Xn,Ce,YZg),(Xn,Ce,YZh),(Xn,Ce,YZi),(Xn, Ce,YZj),(Xn,Ce,YZk),(Xn,Ce,YZl),(Xn,Ce,YZm),(Xn,Ce,YZn),(Xn,Ce,YZo),(Xn,Ce, YZp),(Xn,Ce,YZq),(Xn,Ce,YZr),(Xn,Ce,YZs),(Xn,Ce,YZt),(Xn,Ce,YZu),(Xn,Ce,YZv), (Xn,Ce,YZw),(Xn,Ce,YZx),(Xn,Ce,YZy),(Xn,Ce,YZz),(Xn,Ce,YZaa),(Xn,Ce,YZab), (Xn,Ce,YZac),(Xn,Ce,YZad),(Xn,Ce,YZae),(Xn,Ce,YZaf),(Xn,Ce,YZag),(Xn,Ce,YZah), (Xn,Ce,YZai),(Xn,Ce,YZaj),(Xn,Ce,YZak),(Xn,Ce,YZal),(Xn,Ce,YZam),(Xn,Ce, YZan),(Xn,Ce,YZao),(Xn,Ce,YZap),(Xn,Ce,YZaq),(Xn,Ce,YZar),(Xn,Ce,YZas),(Xn, Ce,YZat),(Xn,Ce,YZau),(Xn,Ce,YZav),(Xn,Ce,YZaw),(Xn,Ce,YZax),(Xn,Ce,YZay),(Xn, Ce,YZaz),(Xn,Ce,YZba),(Xn,Ce,YZbb),(Xn,Ce,YZbc),(Xn,Ce,YZbd),(Xn,Ce,YZbe), (Xn,Ce,YZbf),(Xn,Ce,YZbg),(Xn,Ce,YZbh),(Xn,Ce,YZbi),(Xn,Cc,YZbj),(Xn,Ce,YZbk), (Xn,Ce,YZbl),(Xn,Ce,YZbm),(Xn,Ce,YZbn),(Xn,Ce,YZbo),(Xn,Ce,YZbp),(Xn, Ce,YZbq),(Xn,Ce,YZbr),(Xn,Ce,YZbs),(Xn,Ce,YZbt),(Xn,Ce,YZbu),(Xn,Ce,YZbv),(Xn, Ce,YZbw),(Xn,Ce,YZbx),(Xn,Ce,YZby),(Xn,Ce,YZbz),(Xn,Ce,YZca),(Xn,Ce,YZcb), (Xn,Ce,YZcc),(Xn,Ce,YZcd),(Xn,Cc,YZce),(Xn,Ce,YZcf),(Xn,Ce,YZcg),(Xn,Ce, YZch),(Xn,Ce,YZci),(Xn,Ce,YZcj),(Xn,Ce,YZck),(Xn,Ce,YZcl),(Xn,Ce,YZcm),(Xn, Ce,YZcn),(Xn,Ce,YZco),(Xn,Ce,YZcp),(Xn,Ce,YZcq),(Xn,Ce,YZcr),(Xn,Ce,YZcs),(Xn, Ce,YZct),(Xn,Ce,YZcu),(Xn,Ce,YZcv),(Xn,Ce,YZcw),(Xn,Ce,YZcx),(Xn,Ce,YZcy), (Xn,Ce,YZcz),(Xn,Ce,YZda),(Xn,Ce,YZdb),(Xn,Ce,YZdc),(Xn,Ce,YZdd),(Xn,Ce, YZde),(Xn,Ce,YZdf),(Xn,Ce,YZdg),(Xn,Ce,YZdh),(Xn,Ce,YZdi),(Xn,Ce,YZdj),(Xn, Ce,YZdk),(Xn,Ce,YZdl),(Xn,Ce,YZdm),(Xn,Ce,YZdn),(Xn,Ce,YZdo),(Xn,Ce,YZdp), (Xn,Ce,YZdq),(Xn,Ce,YZdr),(Xn,Ce,YZds),(Xn,Ce,YZdt),(Xn,Ce,YZdu),(Xn,Ce,YZdv), (Xn,Ce,YZdw),(Xn,Ce,YZdx),(Xn,Ce,YZdy),(Xn,Ce,YZdz),(Xn,Ce,YZea),(Xn,Ce, YZeb),(Xn,Ce,YZec),(Xn,Ce,YZed),(Xn,Ce,YZee),(Xn,Ce,YZef),(Xn,Ce,YZeg),(Xn, Ce,YZeh),(Xn,Ce,YZei),(Xn,Ce,YZej),(Xn,Ce,YZek),(Xn,Ce,YZel),(Xn,Ce,YZem),(Xn, Ce,YZen),(Xn,Ce,YZeo),(Xn,Ce,YZep),(Xn,Ce,YZeq),(Xn,Ce,YZer),(Xn,Ce,YZes), (Xn,Ce,YZet),(Xn,Ce,YZeu),(Xn,Ce,YZev),(Xn,Ce,YZew),(Xn,Ce,YZex),(Xn,Ce,YZey), (Xn,Ce,YZez),(Xn,Ce,YZfa),(Xn,Ce,YZfb),(Xn,Ce,YZfc),(Xn,Ce,YZfd),(Xn,Ce, YZfe),(Xn,Ce,YZff),(Xn,Ce,YZfg),(Xn,Ce,YZfh),(Xn,Ce,YZfi),(Xn,Ce,YZfj),(Xn,Ce, YZfk),(Xn,Ce,YZfl),(Xn,Ce,YZfm),(Xn,Ce,YZfn),(Xn,Ce,YZfo),(Xn,Ce,YZfp),(Xn,Ce, YZfq),(Xn,Ce,YZfr),(Xn,Ce,YZfs),(Xn,Ce,YZft),(Xn,Ce,YZfu),(Xn,Ce,YZfv),(Xn,Ce, YZfw),(Xn,Ce,YZfx),(Xn,Ce,YZfy),(Xn,Ce,YZfz),(Xn,Ce,YZga),(Xn,Ce,YZgb),(Xn, Ce,YZgc),(Xn,Ce,YZgd),(Xn,Ce,YZge),(Xn,Ce,YZgf),(Xn,Ce,YZgg),(Xn,Ce,YZgh), (Xn,Ce,YZgi),(Xn,Ce,YZgj),(Xn,Ce,YZgk),(Xn,Ce,YZgl),(Xn,Ce,YZgm),(Xn,Ce,YZgn), (Xn,Ce,YZgo),(Xn,Ce,YZgp),(Xn,Ce,YZgq),(Xn,Ce,YZgr),(Xn,Ce,YZgs),(Xn,Ce, YZgt),(Xn,Cf,YZa),(Xn,Cf,YZb),(Xn,Cf,YZc),(Xn,Cf,YZd),(Xn,Cf,YZe),(Xn,Cf,YZf), (Xn,Cf,YZg),(Xn,Cf,YZh),(Xn,Cf,YZi),(Xn,Cf,YZj),(Xn,Cf,YZk),(Xn,Cf,YZl),(Xn,Cf, YZm),(Xn,Cf,YZn),(Xn,Cf,YZo),(Xn,Cf,YZp),(Xn,Cf,YZq),(Xn,Cf,YZr),(Xn,Cf,YZs), (Xn,Cf,YZt),(Xn,Cf,YZu),(Xn,Cf,YZv),(Xn,Cf,YZw),(Xn,Cf,YZx),(Xn,Cf,YZy),(Xn, Cf,YZz),(Xn,Cf,YZaa),(Xn,Cf,YZab),(Xn,Cf,YZac),(Xn,Cf,YZad),(Xn,Cf,YZae),(Xn, Cf,YZaf),(Xn,Cf,YZag),(Xn,Cf,YZah),(Xn,Cf,YZai),(Xn,Cf,YZaj),(Xn,Cf,YZak),(Xn, Cf,YZal),(Xn,Cf,YZam),(Xn,Cf,YZan),(Xn,Cf,YZao),(Xn,Cf,YZap),(Xn,Cf,YZaq),(Xn, Cf,YZar),(Xn,Cf,YZas),(Xn,Cf,YZat),(Xn,Cf,YZau),(Xn,Cf,YZav),(Xn,Cf,YZaw), (Xn,Cf,YZax),(Xn,Cf,YZay),(Xn,Cf,YZaz),(Xn,Cf,YZba),(Xn,Cf,YZbb),(Xn,Cf,YZbc), (Xn,Cf,YZbd),(Xn,Cf,YZbe),(Xn,Cf,YZbf),(Xn,Cf,YZbg),(Xn,Cf,YZbh),(Xn,Cf,YZbi), (Xn,Cf,YZbj),(Xn,Cf,YZbk),(Xn,Cf,YZbl),(Xn,Cf,YZbm),(Xn,Cf,YZbn),(Xn,Cf,YZbo), (Xn,Cf,YZbp),(Xn,Cf,YZbq),(Xn,Cf,YZbr),(Xn,Cf,YZbs),(Xn,Cf,YZbt),(Xn,Cf, YZbu),(Xn,Cf,YZbv),(Xn,Cf,YZbw),(Xn,Cf,YZbx),(Xn,Cf,YZby),(Xn,Cf,YZbz),(Xn, Cf,YZca),(Xn,Cf,YZcb),(Xn,Cf,YZcc),(Xn,Cf,YZcd),(Xn,Cf,YZce),(Xn,Cf,YZcf),(Xn, Cf,YZcg),(Xn,Cf,YZch),(Xn,Cf,YZci),(Xn,Cf,YZcj),(Xn,Cf,YZck),(Xn,Cf,YZcl),(Xn, Cf,YZcm),(Xn,Cf,YZcn),(Xn,Cf,YZco),(Xn,Cf,YZcp),(Xn,Cf,YZcq),(Xn,Cf,YZcr),(Xn, Cf,YZcs),(Xn,Cf,YZct),(Xn,Cf,YZcu),(Xn,Cf,YZcv),(Xn,Cf,YZcw),(Xn,Cf,YZcx), (Xn,Cf,YZcy),(Xn,Cf,YZcz),(Xn,Cf,YZda),(Xn,Cf,YZdb),(Xn,Cf,YZdc),(Xn,Cf,YZdd), (Xn,Cf,YZde),(Xn,Cf,YZdf),(Xn,Cf,YZdg),(Xn,Cf,YZdh),(Xn,Cf,YZdi),(Xn,Cf,YZdj), (Xn,Cf,YZdk),(Xn,Cf,YZdl),(Xn,Cf,YZdm),(Xn,Cf,YZdn),(Xn,Cf,YZdo),(Xn,Cf, YZdp),(Xn,Cf,YZdq),(Xn,Cf,YZdr),(Xn,Cf,YZds),(Xn,Cf,YZdt),(Xn,Cf,YZdu),(Xn,Cf, YZdv),(Xn,Cf,YZdw),(Xn,Cf,YZdx),(Xn,Cf,YZdy),(Xn,Cf,YZdz),(Xn,Cf,YZea),(Xn, Cf,YZeb),(Xn,Cf,YZec),(Xn,Cf,YZed),(Xn,Cf,YZee),(Xn,Cf,YZef),(Xn,Cf,YZeg),(Xn, Cf,YZeh),(Xn,Cf,YZei),(Xn,Cf,YZej),(Xn,Cf,YZek),(Xn,Cf,YZel),(Xn,Cf,YZem),(Xn, Cf,YZen),(Xn,Cf,YZeo),(Xn,Cf,YZep),(Xn,Cf,YZeq),(Xn,Cf,YZer),(Xn,Cf,YZes),(Xn, Cf,YZet),(Xn,Cf,YZeu),(Xn,Cf,YZev),(Xn,Cf,YZew),(Xn,Cf,YZex),(Xn,Cf,YZey), (Xn,Cf,YZez),(Xn,Cf,YZfa),(Xn,Cf,YZfb),(Xn,Cf,YZfc),(Xn,Cf,YZfd),(Xn,Cf,YZfe), (Xn,Cf,YZff),(Xn,Cf,YZfg),(Xn,Cf,YZfh),(Xn,Cf,YZfi),(Xn,Cf,YZfj),(Xn,Cf,YZfk),(Xn, Cf,YZfl),(Xn,Cf,YZfm),(Xn,Cf,YZfn),(Xn,Cf,YZfo),(Xn,Cf,YZfp),(Xn,Cf,YZfq),(Xn, Cf,YZfr),(Xn,Cf,YZfs),(Xn,Cf,YZft),(Xn,Cf,YZfu),(Xn,Cf,YZfv),(Xn,Cf,YZfw),(Xn, Cf,YZfx),(Xn,Cf,YZfy),(Xn,Cf,YZfz),(Xn,Cf,YZga),(Xn,Cf,YZgb),(Xn,Cf,YZgc), (Xn,Cf,YZgd),(Xn,Cf,YZge),(Xn,Cf,YZgf),(Xn,Cf,YZgg),(Xn,Cf,YZgh),(Xn,Cf,YZgi), (Xn,Cf,YZgj),(Xn,Cf,YZgk),(Xn,Cf,YZgl),(Xn,Cf,YZgm),(Xn,Cf,YZgn),(Xn,Cf,YZgo), (Xn,Cf,YZgp),(Xn,Cf,YZgq),(Xn,Cf,YZgr),(Xn,Cf,YZgs),(Xn,Cf,YZgt), (Xo,Ca,YZa),(Xo,Ca,YZb),(Xo,Ca,YZc),(Xo,Ca,YZd),(Xo,Ca,YZe),(Xo,Ca,YZf),(Xo, Ca,YZg),(Xo,Ca,YZh),(Xo,Ca,YZi),(Xo,Ca,YZj),(Xo,Ca,YZk),(Xo,Ca,YZl),(Xo,Ca,YZm), (Xo,Ca,YZn),(Xo,Ca,YZo),(Xo,Ca,YZp),(Xo,Ca,YZq),(Xo,Ca,YZr),(Xo,Ca,YZs), (Xo,Ca,YZt),(Xo,Ca,YZu),(Xo,Ca,YZv),(Xo,Ca,YZw),(Xo,Ca,YZx),(Xo,Ca,YZy),(Xo, Ca,YZz),(Xo,Ca,YZaa),(Xo,Ca,YZab),(Xo,Ca,YZac),(Xo,Ca,YZad),(Xo,Ca,YZae),(Xo, Ca,YZaf),(Xo,Ca,YZag),(Xo,Ca,YZah),(Xo,Ca,YZai),(Xo,Ca,YZaj),(Xo,Ca,YZak), (Xo,Ca,YZal),(Xo,Ca,YZam),(Xo,Ca,YZan),(Xo,Ca,YZao),(Xo,Ca,YZap),(Xo,Ca,YZaq), (Xo,Ca,YZar),(Xo,Ca,YZas),(Xo,Ca,YZat),(Xo,Ca,YZau),(Xo,Ca,YZav),(Xo,Ca, YZaw),(Xo,Ca,YZax),(Xo,Ca,YZay),(Xo,Ca,YZaz),(Xo,Ca,YZba),(Xo,Ca,YZbb),(Xo, Ca,YZbc),(Xo,Ca,YZbd),(Xo,Ca,YZbe),(Xo,Ca,YZbf),(Xo,Ca,YZbg),(Xo,Ca,YZbh),(Xo, Ca,YZbi),(Xo,Ca,YZbj),(Xo,Ca,YZbk),(Xo,Ca,YZbl),(Xo,Ca,YZbm),(Xo,Ca,YZbn), (Xo,Ca,YZbo),(Xo,Ca,YZbp),(Xo,Ca,YZbq),(Xo,Ca,YZbr),(Xo,Ca,YZbs),(Xo,Ca, YZbt),(Xo,Ca,YZbu),(Xo,Ca,YZbv),(Xo,Ca,YZbw),(Xo,Ca,YZbx),(Xo,Ca,YZby),(Xo, Ca,YZbz),(Xo,Ca,YZca),(Xo,Ca,YZcb),(Xo,Ca,YZcc),(Xo,Ca,YZcd),(Xo,Ca,YZce),(Xo, Ca,YZcf),(Xo,Ca,YZcg),(Xo,Ca,YZch),(Xo,Ca,YZci),(Xo,Ca,YZcj),(Xo,Ca,YZck), (Xo,Ca,YZcl),(Xo,Ca,YZcm),(Xo,Ca,YZcn),(Xo,Ca,YZco),(Xo,Ca,YZcp),(Xo,Ca,YZcq), (Xo,Ca,YZcr),(Xo,Ca,YZcs),(Xo,Ca,YZct),(Xo,Ca,YZcu),(Xo,Ca,YZcv),(Xo,Ca, YZcw),(Xo,Ca,YZcx),(Xo,Ca,YZcy),(Xo,Ca,YZcz),(Xo,Ca,YZda),(Xo,Ca,YZdb),(Xo, Ca,YZdc),(Xo,Ca,YZdd),(Xo,Ca,YZde),(Xo,Ca,YZdf),(Xo,Ca,YZdg),(Xo,Ca,YZdh),(Xo, Ca,YZdi),(Xo,Ca,YZdj),(Xo,Ca,YZdk),(Xo,Ca,YZdl),(Xo,Ca,YZdm),(Xo,Ca,YZdn), (Xo,Ca,YZdo),(Xo,Ca,YZdp),(Xo,Ca,YZdq),(Xo,Ca,YZdr),(Xo,Ca,YZds),(Xo,Ca, YZdt),(Xo,Ca,YZdu),(Xo,Ca,YZdv),(Xo,Ca,YZdw),(Xo,Ca,YZdx),(Xo,Ca,YZdy),(Xo, Ca,YZdz),(Xo,Ca,YZea),(Xo,Ca,YZeb),(Xo,Ca,YZec),(Xo,Ca,YZed),(Xo,Ca,YZee),(Xo, Ca,YZef),(Xo,Ca,YZeg),(Xo,Ca,YZeh),(Xo,Ca,YZei),(Xo,Ca,YZej),(Xo,Ca,YZek), (Xo,Ca,YZel),(Xo,Ca,YZem),(Xo,Ca,YZen),(Xo,Ca,YZeo),(Xo,Ca,YZep),(Xo,Ca,YZeq), (Xo,Ca,YZer),(Xo,Ca,YZes),(Xo,Ca,YZet),(Xo,Ca,YZeu),(Xo,Ca,YZev),(Xo,Ca, YZew),(Xo,Ca,YZex),(Xo,Ca,YZey),(Xo,Ca,YZez),(Xo,Ca,YZfa),(Xo,Ca,YZfb),(Xo, Ca,YZfc),(Xo,Ca,YZfd),(Xo,Ca,YZfe),(Xo,Ca,YZff),(Xo,Ca,YZfg),(Xo,Ca,YZfh),(Xo, Ca,YZfi),(Xo,Ca,YZfj),(Xo,Ca,YZfk),(Xo,Ca,YZfl),(Xo,Ca,YZfm),(Xo,Ca,YZfn),(Xo, Ca,YZfo),(Xo,Ca,YZfp),(Xo,Ca,YZfq),(Xo,Ca,YZfr),(Xo,Ca,YZfs),(Xo,Ca,YZft),(Xo, Ca,YZfu),(Xo,Ca,YZfv),(Xo,Ca,YZfw),(Xo,Ca,YZfx),(Xo,Ca,YZfy),(Xo,Ca,YZfz),(Xo, Ca,YZga),(Xo,Ca,YZgb),(Xo,Ca,YZgc),(Xo,Ca,YZgd),(Xo,Ca,YZge),(Xo,Ca,YZgf), (Xo,Ca,YZgg),(Xo,Ca,YZgh),(Xo,Ca,YZgi),(Xo,Ca,YZgj),(Xo,Ca,YZgk),(Xo,Ca,YZgl), (Xo,Ca,YZgm),(Xo,Ca,YZgn),(Xo,Ca,YZgo),(Xo,Ca,YZgp),(Xo,Ca,YZgq),(Xo, Ca,YZgr),(Xo,Ca,YZgs),(Xo,Ca,YZgt),(Xo,Cb,YZa),(Xo,Cb,YZb),(Xo,Cb,YZc),(Xo, Cb,YZd),(Xo,Cb,YZe),(Xo,Cb,YZf),(Xo,Cb,YZg),(Xo,Cb,YZh),(Xo,Cb,YZi),(Xo,Cb, YZj),(Xo,Cb,YZk),(Xo,Cb,YZl),(Xo,Cb,YZm),(Xo,Cb,YZn),(Xo,Cb,YZo),(Xo,Cb,YZp), (Xo,Cb,YZq),(Xo,Cb,YZr),(Xo,Cb,YZs),(Xo,Cb,YZt),(Xo,Cb,YZu),(Xo,Cb,YZv),(Xo, Cb,YZw),(Xo,Cb,YZx),(Xo,Cb,YZy),(Xo,Cb,YZz),(Xo,Cb,YZaa),(Xo,Cb,YZab),(Xo, Cb,YZac),(Xo,Cb,YZad),(Xo,Cb,YZae),(Xo,Cb,YZaf),(Xo,Cb,YZag),(Xo,Cb,YZah), (Xo,Cb,YZai),(Xo,Cb,YZaj),(Xo,Cb,YZak),(Xo,Cb,YZal),(Xo,Cb,YZam),(Xo,Cb, YZan),(Xo,Cb,YZao),(Xo,Cb,YZap),(Xo,Cb,YZaq),(Xo,Cb,YZar),(Xo,Cb,YZas),(Xo, Cb,YZat),(Xo,Cb,YZau),(Xo,Cb,YZav),(Xo,Cb,YZaw),(Xo,Cb,YZax),(Xo,Cb,YZay), (Xo,Cb,YZaz),(Xo,Cb,YZba),(Xo,Cb,YZbb),(Xo,Cb,YZbc),(Xo,Cb,YZbd),(Xo,Cb,YZbe), (Xo,Cb,YZbf),(Xo,Cb,YZbg),(Xo,Cb,YZbh),(Xo,Cb,YZbi),(Xo,Cb,YZbj),(Xo,Cb, YZbk),(Xo,Cb,YZbl),(Xo,Cb,YZbm),(Xo,Cb,YZbn),(Xo,Cb,YZbo),(Xo,Cb,YZbp),(Xo, Cb,YZbq),(Xo,Cb,YZbr),(Xo,Cb,YZbs),(Xo,Cb,YZbt),(Xo,Cb,YZbu),(Xo,Cb,YZbv), (Xo,Cb,YZbw),(Xo,Cb,YZbx),(Xo,Cb,YZby),(Xo,Cb,YZbz),(Xo,Cb,YZca),(Xo,Cb, YZcb),(Xo,Cb,YZcc),(Xo,Cb,YZcd),(Xo,Cb,YZce),(Xo,Cb,YZcf),(Xo,Cb,YZcg),(Xo, Cb,YZch),(Xo,Cb,YZci),(Xo,Cb,YZcj),(Xo,Cb,YZck),(Xo,Cb,YZcl),(Xo,Cb,YZcm),(Xo, Cb,YZcn),(Xo,Cb,YZco),(Xo,Cb,YZcp),(Xo,Cb,YZcq),(Xo,Cb,YZcr),(Xo,Cb,YZcs), (Xo,Cb,YZct),(Xo,Cb,YZcu),(Xo,Cb,YZcv),(Xo,Cb,YZcw),(Xo,Cb,YZcx),(Xo,Cb, YZcy),(Xo,Cb,YZcz),(Xo,Cb,YZda),(Xo,Cb,YZdb),(Xo,Cb,YZdc),(Xo,Cb,YZdd),(Xo, Cb,YZde),(Xo,Cb,YZdf),(Xo,Cb,YZdg),(Xo,Cb,YZdh),(Xo,Cb,YZdi),(Xo,Cb,YZdj),(Xo, Cb,YZdk),(Xo,Cb,YZdl),(Xo,Cb,YZdm),(Xo,Cb,YZdn),(Xo,Cb,YZdo),(Xo,Cb,YZdp), (Xo,Cb,YZdq),(Xo,Cb,YZdr),(Xo,Cb,YZds),(Xo,Cb,YZdt),(Xo,Cb,YZdu),(Xo,Cb, YZdv),(Xo,Cb,YZdw),(Xo,Cb,YZdx),(Xo,Cb,YZdy),(Xo,Cb,YZdz),(Xo,Cb,YZea),(Xo, Cb,YZeb),(Xo,Cb,YZec),(Xo,Cb,YZed),(Xo,Cb,YZee),(Xo,Cb,YZef),(Xo,Cb,YZeg), (Xo,Cb,YZeh),(Xo,Cb,YZei),(Xo,Cb,YZej),(Xo,Cb,YZek),(Xo,Cb,YZel),(Xo,Cb,YZem), (Xo,Cb,YZen),(Xo,Cb,YZeo),(Xo,Cb,YZep),(Xo,Cb,YZeq),(Xo,Cb,YZer),(Xo,Cb, YZes),(Xo,Cb,YZet),(Xo,Cb,YZeu),(Xo,Cb,YZev),(Xo,Cb,YZew),(Xo,Cb,YZex),(Xo, Cb,YZey),(Xo,Cb,YZez),(Xo,Cb,YZfa),(Xo,Cb,YZfb),(Xo,Cb,YZfc),(Xo,Cb,YZfd),(Xo, Cb,YZfe),(Xo,Cb,YZff),(Xo,Cb,YZfg),(Xo,Cb,YZfh),(Xo,Cb,YZfi),(Xo,Cb,YZfj), (Xo,Cb,YZfk),(Xo,Cb,YZfl),(Xo,Cb,YZfm),(Xo,Cb,YZfn),(Xo,Cb,YZfo),(Xo,Cb,YZfp), (Xo,Cb,YZfq),(Xo,Cb,YZfr),(Xo,Cb,YZfs),(Xo,Cb,YZft),(Xo,Cb,YZfu),(Xo,Cb,YZfv), (Xo,Cb,YZfw),(Xo,Cb,YZfx),(Xo,Cb,YZfy),(Xo,Cb,YZfz),(Xo,Cb,YZga),(Xo,Cb, YZgb),(Xo,Cb,YZgc),(Xo,Cb,YZgd),(Xo,Cb,YZge),(Xo,Cb,YZgf),(Xo,Cb,YZgg),(Xo, Cb,YZgh),(Xo,Cb,YZgi),(Xo,Cb,YZgj),(Xo,Cb,YZgk),(Xo,Cb,YZgl),(Xo,Cb,YZgm), (Xo,Cb,YZgn),(Xo,Cb,YZgo),(Xo,Cb,YZgp),(Xo,Cb,YZgq),(Xo,Cb,YZgr),(Xo,Cb,YZgs), (Xo,Cb,YZgt),(Xo,Cc,YZa),(Xo,Cc,YZb),(Xo,Cc,YZc),(Xo,Cc,YZd),(Xo,Cc,YZe), (Xo,Cc,YZf),(Xo,Cc,YZg),(Xo,Cc,YZh),(Xo,Cc,YZi),(Xo,Cc,YZj),(Xo,Cc,YZk),(Xo, Cc,YZl),(Xo,Cc,YZm),(Xo,Cc,YZn),(Xo,Cc,YZo),(Xo,Cc,YZp),(Xo,Cc,YZq),(Xo,Cc, YZr),(Xo,Cc,YZs),(Xo,Cc,YZt),(Xo,Cc,YZu),(Xo,Cc,YZv),(Xo,Cc,YZw),(Xo,Cc,YZx), (Xo,Cc,YZy),(Xo,Cc,YZz),(Xo,Cc,YZaa),(Xo,Cc,YZab),(Xo,Cc,YZac),(Xo,Cc,YZad), (Xo,Cc,YZae),(Xo,Cc,YZaf),(Xo,Cc,YZag),(Xo,Cc,YZah),(Xo,Cc,YZai),(Xo,Cc, YZaj),(Xo,Cc,YZak),(Xo,Cc,YZal),(Xo,Cc,YZam),(Xo,Cc,YZan),(Xo,Cc,YZao),(Xo, Cc,YZap),(Xo,Cc,YZaq),(Xo,Cc,YZar),(Xo,Cc,YZas),(Xo,Cc,YZat),(Xo,Cc,YZau),(Xo, Ce,YZav),(Xo,Cc,YZaw),(Xo,Cc,YZax),(Xo,Cc,YZay),(Xo,Cc,YZaz),(Xo,Cc,YZba), (Xo,Cc,YZbb),(Xo,Cc,YZbc),(Xo,Cc,YZbd),(Xo,Cc,YZbe),(Xo,Cc,YZbf),(Xo,Cc,YZbg), (Xo,Cc,YZbh),(Xo,Cc,YZbi),(Xo,Cc,YZbj),(Xo,Cc,YZbk),(Xo,Cc,YZbl),(Xo,Cc, YZbm),(Xo,Cc,YZbn),(Xo,Cc,YZbo),(Xo,Cc,YZbp),(Xo,Cc,YZbq),(Xo,Cc,YZbr),(Xo, Cc,YZbs),(Xo,Cc,YZbt),(Xo,Cc,YZbu),(Xo,Cc,YZbv),(Xo,Cc,YZbw),(Xo,Cc,YZbx), (Xo,Cc,YZby),(Xo,Cc,YZbz),(Xo,Cc,YZca),(Xo,Cc,YZcb),(Xo,Cc,YZcc),(Xo,Cc,YZcd), (Xo,Cc,YZce),(Xo,Cc,YZcf),(Xo,Cc,YZcg),(Xo,Cc,YZch),(Xo,Cc,YZci),(Xo,Cc, YZcj),(Xo,Cc,YZck),(Xo,Cc,YZcl),(Xo,Cc,YZcm),(Xo,Cc,YZcn),(Xo,Cc,YZco),(Xo, Cc,YZcp),(Xo,Cc,YZcq),(Xo,Cc,YZcr),(Xo,Cc,YZcs),(Xo,Cc,YZct),(Xo,Cc,YZcu),(Xo, Cc,YZcv),(Xo,Cc,YZcw),(Xo,Cc,YZcx),(Xo,Cc,YZcy),(Xo,Cc,YZcz),(Xo,Cc,YZda), (Xo,Cc,YZdb),(Xo,Cc,YZdc),(Xo,Cc,YZdd),(Xo,Cc,YZde),(Xo,Cc,YZdf),(Xo,Cc,YZdg), (Xo,Cc,YZdh),(Xo,Cc,YZdi),(Xo,Cc,YZdj),(Xo,Cc,YZdk),(Xo,Cc,YZdl),(Xo,Ce, YZdm),(Xo,Cc,YZdn),(Xo,Cc,YZdo),(Xo,Cc,YZdp),(Xo,Cc,YZdq),(Xo,Cc,YZdr),(Xo, Cc,YZds),(Xo,Cc,YZdt),(Xo,Cc,YZdu),(Xo,Cc,YZdv),(Xo,Cc,YZdw),(Xo,Cc,YZdx), (Xo,Cc,YZdy),(Xo,Cc,YZdz),(Xo,Cc,YZea),(Xo,Cc,YZeb),(Xo,Cc,YZec),(Xo,Cc,YZed), (Xo,Cc,YZee),(Xo,Cc,YZef),(Xo,Cc,YZeg),(Xo,Cc,YZeh),(Xo,Cc,YZei),(Xo,Cc, YZej),(Xo,Cc,YZek),(Xo,Cc,YZel),(Xo,Cc,YZem),(Xo,Cc,YZen),(Xo,Cc,YZeo),(Xo, Cc,YZep),(Xo,Cc,YZeq),(Xo,Cc,YZer),(Xo,Cc,YZes),(Xo,Cc,YZet),(Xo,Cc,YZeu),(Xo, Cc,YZev),(Xo,Cc,YZew),(Xo,Cc,YZex),(Xo,Cc,YZey),(Xo,Cc,YZez),(Xo,Cc,YZfa), (Xo,Cc,YZfb),(Xo,Cc,YZfc),(Xo,Cc,YZfd),(Xo,Cc,YZfe),(Xo,Cc,YZff),(Xo,Cc,YZfg), (Xo,Cc,YZfh),(Xo,Cc,YZfi),(Xo,Cc,YZfj),(Xo,Cc,YZfk),(Xo,Cc,YZfl),(Xo,Cc,YZfm), (Xo,Cc,YZfn),(Xo,Cc,YZfo),(Xo,Cc,YZfp),(Xo,Cc,YZfq),(Xo,Cc,YZfr),(Xo,Cc,YZfs), (Xo,Cc,YZft),(Xo,Cc,YZfu),(Xo,Cc,YZfv),(Xo,Cc,YZfw),(Xo,Cc,YZfx),(Xo,Cc, YZfy),(Xo,Cc,YZfz),(Xo,Cc,YZga),(Xo,Cc,YZgb),(Xo,Cc,YZgc),(Xo,Cc,YZgd),(Xo, Cc,YZge),(Xo,Cc,YZgf),(Xo,Cc,YZgg),(Xo,Cc,YZgh),(Xo,Cc,YZgi),(Xo,Cc,YZgj),(Xo, Cc,YZgk),(Xo,Cc,YZgl),(Xo,Cc,YZgm),(Xo,Cc,YZgn),(Xo,Cc,YZgo),(Xo,Cc,YZgp), (Xo,Cc,YZgq),(Xo,Cc,YZgr),(Xo,Cc,YZgs),(Xo,Cc,YZgt),(Xo,Cd,YZa),(Xo,Cd,YZb), (Xo,Cd,YZc),(Xo,Cd,YZd),(Xo,Cd,YZe),(Xo,Cd,YZf),(Xo,Cd,YZg),(Xo,Cd,YZh), (Xo,Cd,YZi),(Xo,Cd,YZj),(Xo,Cd,YZk),(Xo,Cd,YZl),(Xo,Cd,YZm),(Xo,Cd,YZn),(Xo, Cd,YZo),(Xo,Cd,YZp),(Xo,Cd,YZq),(Xo,Cd,YZr),(Xo,Cd,YZs),(Xo,Cd,YZt),(Xo,Cd, YZu),(Xo,Cd,YZv),(Xo,Cd,YZw),(Xo,Cd,YZx),(Xo,Cd,YZy),(Xo,Cd,YZz),(Xo,Cd, YZaa),(Xo,Cd,YZab),(Xo,Cd,YZac),(Xo,Cd,YZad),(Xo,Cd,YZae),(Xo,Cd,YZaf),(Xo, Cd,YZag),(Xo,Cd,YZah),(Xo,Cd,YZai),(Xo,Cd,YZaj),(Xo,Cd,YZak),(Xo,Cd,YZal),(Xo, Cd,YZam),(Xo,Cd,YZan),(Xo,Cd,YZao),(Xo,Cd,YZap),(Xo,Cd,YZaq),(Xo,Cd,YZar), (Xo,Cd,YZas),(Xo,Cd,YZat),(Xo,Cd,YZau),(Xo,Cd,YZav),(Xo,Cd,YZaw),(Xo,Cd, YZax),(Xo,Cd,YZay),(Xo,Cd,YZaz),(Xo,Cd,YZba),(Xo,Cd,YZbb),(Xo,Cd,YZbc),(Xo, Cd,YZbd),(Xo,Cd,YZbe),(Xo,Cd,YZbf),(Xo,Cd,YZbg),(Xo,Cd,YZbh),(Xo,Cd,YZbi), (Xo,Cd,YZbj),(Xo,Cd,YZbk),(Xo,Cd,YZbl),(Xo,Cd,YZbm),(Xo,Cd,YZbn),(Xo,Cd,YZbo), (Xo,Cd,YZbp),(Xo,Cd,YZbq),(Xo,Cd,YZbr),(Xo,Cd,YZbs),(Xo,Cd,YZbt),(Xo,Cd, YZbu),(Xo,Cd,YZbv),(Xo,Cd,YZbw),(Xo,Cd,YZbx),(Xo,Cd,YZby),(Xo,Cd,YZbz),(Xo, Cd,YZca),(Xo,Cd,YZcb),(Xo,Cd,YZcc),(Xo,Cd,YZcd),(Xo,Cd,YZce),(Xo,Cd,YZcf), (Xo,Cd,YZcg),(Xo,Cd,YZch),(Xo,Cd,YZci),(Xo,Cd,YZcj),(Xo,Cd,YZck),(Xo,Cd,YZcl), (Xo,Cd,YZcm),(Xo,Cd,YZcn),(Xo,Cd,YZco),(Xo,Cd,YZcp),(Xo,Cd,YZcq),(Xo, Cd,YZcr),(Xo,Cd,YZcs),(Xo,Cd,YZct),(Xo,Cd,YZcu),(Xo,Cd,YZcv),(Xo,Cd,YZcw),(Xo, Cd,YZcx),(Xo,Cd,YZcy),(Xo,Cd,YZcz),(Xo,Cd,YZda),(Xo,Cd,YZdb),(Xo,Cd,YZdc), (Xo,Cd,YZdd),(Xo,Cd,YZde),(Xo,Cd,YZdf),(Xo,Cd,YZdg),(Xo,Cd,YZdh),(Xo,Cd, YZdi),(Xo,Cd,YZdj),(Xo,Cd,YZdk),(Xo,Cd,YZdl),(Xo,Cd,YZdm),(Xo,Cd,YZdn),(Xo, Cd,YZdo),(Xo,Cd,YZdp),(Xo,Cd,YZdq),(Xo,Cd,YZdr),(Xo,Cd,YZds),(Xo,Cd,YZdt), (Xo,Cd,YZdu),(Xo,Cd,YZdv),(Xo,Cd,YZdw),(Xo,Cd,YZdx),(Xo,Cd,YZdy),(Xo,Cd, YZdz),(Xo,Cd,YZea),(Xo,Cd,YZeb),(Xo,Cd,YZec),(Xo,Cd,YZed),(Xo,Cd,YZee),(Xo, Cd,YZef),(Xo,Cd,YZeg),(Xo,Cd,YZeh),(Xo,Cd,YZei),(Xo,Cd,YZej),(Xo,Cd,YZek),(Xo, Cd,YZel),(Xo,Cd,YZem),(Xo,Cd,YZen),(Xo,Cd,YZeo),(Xo,Cd,YZep),(Xo,Cd,YZeq), (Xo,Cd,YZer),(Xo,Cd,YZes),(Xo,Cd,YZet),(Xo,Cd,YZeu),(Xo,Cd,YZev),(Xo,Cd, YZew),(Xo,Cd,YZex),(Xo,Cd,YZey),(Xo,Cd,YZez),(Xo,Cd,YZfa),(Xo,Cd,YZfb),(Xo, Cd,YZfc),(Xo,Cd,YZfd),(Xo,Cd,YZfe),(Xo,Cd,YZff),(Xo,Cd,YZfg),(Xo,Cd,YZfh),(Xo, Cd,YZfi),(Xo,Cd,YZfj),(Xo,Cd,YZfk),(Xo,Cd,YZfl),(Xo,Cd,YZfm),(Xo,Cd,YZfn),(Xo, Cd,YZfo),(Xo,Cd,YZfp),(Xo,Cd,YZfq),(Xo,Cd,YZfr),(Xo,Cd,YZfs),(Xo,Cd,YZft), (Xo,Cd,YZfu),(Xo,Cd,YZfv),(Xo,Cd,YZfw),(Xo,Cd,YZfx),(Xo,Cd,YZfy),(Xo,Cd,YZfz), (Xo,Cd,YZga),(Xo,Cd,YZgb),(Xo,Cd,YZgc),(Xo,Cd,YZgd),(Xo,Cd,YZge),(Xo,Cd, YZgf),(Xo,Cd,YZgg),(Xo,Cd,YZgh),(Xo,Cd,YZgi),(Xo,Cd,YZgj),(Xo,Cd,YZgk),(Xo, Cd,YZgl),(Xo,Cd,YZgm),(Xo,Cd,YZgn),(Xo,Cd,YZgo),(Xo,Cd,YZgp),(Xo,Cd,YZgq), (Xo,Cd,YZgr),(Xo,Cd,YZgs),(Xo,Cd,YZgt),(Xo,Ce,YZa),(Xo,Ce,YZb),(Xo,Ce,YZc), (Xo,Ce,YZd),(Xo,Ce,YZe),(Xo,Ce,YZf),(Xo,Ce,YZg),(Xo,Ce,YZh),(Xo,Ce,YZi),(Xo, Ce,YZj),(Xo,Ce,YZk),(Xo,Ce,YZl),(Xo,Ce,YZm),(Xo,Ce,YZn),(Xo,Ce,YZo),(Xo,Ce, YZp),(Xo,Ce,YZq),(Xo,Ce,YZr),(Xo,Ce,YZs),(Xo,Ce,YZt),(Xo,Ce,YZu),(Xo,Ce,YZv), (Xo,Ce,YZw),(Xo,Ce,YZx),(Xo,Ce,YZy),(Xo,Ce,YZz),(Xo,Ce,YZaa),(Xo,Ce,YZab), (Xo,Ce,YZac),(Xo,Ce,YZad),(Xo,Ce,YZae),(Xo,Ce,YZaf),(Xo,Ce,YZag),(Xo,Ce,YZah), (Xo,Ce,YZai),(Xo,Ce,YZaj),(Xo,Ce,YZak),(Xo,Ce,YZal),(Xo,Ce,YZam),(Xo,Ce, YZan),(Xo,Ce,YZao),(Xo,Ce,YZap),(Xo,Ce,YZaq),(Xo,Ce,YZar),(Xo,Ce,YZas),(Xo, Ce,YZat),(Xo,Ce,YZau),(Xo,Ce,YZav),(Xo,Ce,YZaw),(Xo,Ce,YZax),(Xo,Ce,YZay),(Xo, Ce,YZaz),(Xo,Ce,YZba),(Xo,Ce,YZbb),(Xo,Ce,YZbc),(Xo,Ce,YZbd),(Xo,Ce,YZbe), (Xo,Ce,YZbf),(Xo,Ce,YZbg),(Xo,Ce,YZbh),(Xo,Ce,YZbi),(Xo,Ce,YZbj),(Xo,Ce,YZbk), (Xo,Ce,YZbl),(Xo,Ce,YZbm),(Xo,Ce,YZbn),(Xo,Ce,YZbo),(Xo,Ce,YZbp),(Xo, Ce,YZbq),(Xo,Ce,YZbr),(Xo,Ce,YZbs),(Xo,Ce,YZbt),(Xo,Ce,YZbu),(Xo,Ce,YZbv),(Xo, Ce,YZbw),(Xo,Ce,YZbx),(Xo,Ce,YZby),(Xo,Ce,YZbz),(Xo,Ce,YZca),(Xo,Ce,YZcb), (Xo,Ce,YZcc),(Xo,Ce,YZcd),(Xo,Ce,YZce),(Xo,Ce,YZcf),(Xo,Ce,YZcg),(Xo,Ce, YZch),(Xo,Ce,YZci),(Xo,Ce,YZcj),(Xo,Ce,YZck),(Xo,Ce,YZcl),(Xo,Ce,YZcm),(Xo, Ce,YZcn),(Xo,Ce,YZco),(Xo,Ce,YZcp),(Xo,Ce,YZcq),(Xo,Ce,YZcr),(Xo,Ce,YZcs),(Xo, Ce,YZct),(Xo,Ce,YZcu),(Xo,Ce,YZcv),(Xo,Ce,YZcw),(Xo,Ce,YZcx),(Xo,Ce,YZcy), (Xo,Ce,YZcz),(Xo,Ce,YZda),(Xo,Ce,YZdb),(Xo,Ce,YZdc),(Xo,Ce,YZdd),(Xo,Ce, YZde),(Xo,Ce,YZdf),(Xo,Ce,YZdg),(Xo,Ce,YZdh),(Xo,Ce,YZdi),(Xo,Ce,YZdj),(Xo, Ce,YZdk),(Xo,Ce,YZdl),(Xo,Ce,YZdm),(Xo,Ce,YZdn),(Xo,Ce,YZdo),(Xo,Ce,YZdp), (Xo,Ce,YZdq),(Xo,Ce,YZdr),(Xo,Ce,YZds),(Xo,Ce,YZdt),(Xo,Ce,YZdu),(Xo,Ce,YZdv), (Xo,Ce,YZdw),(Xo,Ce,YZdx),(Xo,Ce,YZdy),(Xo,Ce,YZdz),(Xo,Ce,YZea),(Xo,Ce, YZeb),(Xo,Ce,YZec),(Xo,Ce,YZed),(Xo,Ce,YZee),(Xo,Ce,YZef),(Xo,Ce,YZeg),(Xo, Ce,YZeh),(Xo,Ce,YZei),(Xo,Ce,YZej),(Xo,Ce,YZek),(Xo,Ce,YZel),(Xo,Ce,YZem),(Xo, Ce,YZen),(Xo,Ce,YZeo),(Xo,Ce,YZep),(Xo,Ce,YZeq),(Xo,Ce,YZer),(Xo,Ce,YZes), (Xo,Ce,YZet),(Xo,Ce,YZeu),(Xo,Ce,YZev),(Xo,Ce,YZew),(Xo,Ce,YZex),(Xo,Ce,YZey), (Xo,Ce,YZez),(Xo,Ce,YZfa),(Xo,Ce,YZfb),(Xo,Ce,YZfc),(Xo,Ce,YZfd),(Xo,Ce, YZfe),(Xo,Ce,YZff),(Xo,Ce,YZfg),(Xo,Ce,YZfh),(Xo,Ce,YZfi),(Xo,Ce,YZfj),(Xo,Ce, YZfk),(Xo,Ce,YZfl),(Xo,Ce,YZfm),(Xo,Ce,YZfn),(Xo,Ce,YZfo),(Xo,Ce,YZfp),(Xo,Ce, YZfq),(Xo,Ce,YZfr),(Xo,Ce,YZfs),(Xo,Ce,YZft),(Xo,Ce,YZfu),(Xo,Ce,YZfv),(Xo,Ce, YZfw),(Xo,Ce,YZfx),(Xo,Ce,YZfy),(Xo,Ce,YZfz),(Xo,Ce,YZga),(Xo,Ce,YZgb),(Xo, Ce,YZgc),(Xo,Ce,YZgd),(Xo,Ce,YZge),(Xo,Ce,YZgf),(Xo,Ce,YZgg),(Xo,Ce,YZgh), (Xo,Ce,YZgi),(Xo,Ce,YZgj),(Xo,Ce,YZgk),(Xo,Ce,YZgl),(Xo,Ce,YZgm),(Xo,Ce,YZgn), (Xo,Ce,YZgo),(Xo,Ce,YZgp),(Xo,Ce,YZgq),(Xo,Ce,YZgr),(Xo,Ce,YZgs),(Xo,Ce, YZgt),(Xo,Cf,YZa),(Xo,Cf,YZb),(Xo,Cf,YZc),(Xo,Cf,YZd),(Xo,Cf,YZe),(Xo,Cf,YZf), (Xo,Cf,YZg),(Xo,Cf,YZh),(Xo,Cf,YZi),(Xo,Cf,YZj),(Xo,Cf,YZk),(Xo,Cf,YZl),(Xo,Cf, YZm),(Xo,Cf,YZn),(Xo,Cf,YZo),(Xo,Cf,YZp),(Xo,Cf,YZq),(Xo,Cf,YZr),(Xo,Cf,YZs), (Xo,Cf,YZt),(Xo,Cf,YZu),(Xo,Cf,YZv),(Xo,Cf,YZw),(Xo,Cf,YZx),(Xo,Cf,YZy),(Xo, Cf,YZz),(Xo,Cf,YZaa),(Xo,Cf,YZab),(Xo,Cf,YZac),(Xo,Cf,YZad),(Xo,Cf,YZae),(Xo, Cf,YZaf),(Xo,Cf,YZag),(Xo,Cf,YZah),(Xo,Cf,YZai),(Xo,Cf,YZaj),(Xo,Cf,YZak),(Xo, Cf,YZal),(Xo,Cf,YZam),(Xo,Cf,YZan),(Xo,Cf,YZao),(Xo,Cf,YZap),(Xo,Cf,YZaq),(Xo, Cf,YZar),(Xo,Cf,YZas),(Xo,Cf,YZat),(Xo,Cf,YZau),(Xo,Cf,YZav),(Xo,Cf,YZaw), (Xo,Cf,YZax),(Xo,Cf,YZay),(Xo,Cf,YZaz),(Xo,Cf,YZba),(Xo,Cf,YZbb),(Xo,Cf,YZbc), (Xo,Cf,YZbd),(Xo,Cf,YZbe),(Xo,Cf,YZbf),(Xo,Cf,YZbg),(Xo,Cf,YZbh),(Xo,Cf,YZbi), (Xo,Cf,YZbj),(Xo,Cf,YZbk),(Xo,Cf,YZbl),(Xo,Cf,YZbm),(Xo,Cf,YZbn),(Xo,Cf,YZbo), (Xo,Cf,YZbp),(Xo,Cf,YZbq),(Xo,Cf,YZbr),(Xo,Cf,YZbs),(Xo,Cf,YZbt),(Xo,Cf, YZbu),(Xo,Cf,YZbv),(Xo,Cf,YZbw),(Xo,Cf,YZbx),(Xo,Cf,YZby),(Xo,Cf,YZbz),(Xo, Cf,YZca),(Xo,Cf,YZcb),(Xo,Cf,YZcc),(Xo,Cf,YZcd),(Xo,Cf,YZce),(Xo,Cf,YZcf),(Xo, Cf,YZcg),(Xo,Cf,YZch),(Xo,Cf,YZci),(Xo,Cf,YZcj),(Xo,Cf,YZck),(Xo,Cf,YZcl),(Xo, Cf,YZcm),(Xo,Cf,YZcn),(Xo,Cf,YZco),(Xo,Cf,YZcp),(Xo,Cf,YZcq),(Xo,Cf,YZcr),(Xo, Cf,YZcs),(Xo,Cf,YZct),(Xo,Cf,YZcu),(Xo,Cf,YZcv),(Xo,Cf,YZcw),(Xo,Cf,YZcx), (Xo,Cf,YZcy),(Xo,Cf,YZcz),(Xo,Cf,YZda),(Xo,Cf,YZdb),(Xo,Cf,YZdc),(Xo,Cf,YZdd), (Xo,Cf,YZde),(Xo,Cf,YZdf),(Xo,Cf,YZdg),(Xo,Cf,YZdh),(Xo,Cf,YZdi),(Xo,Cf,YZdj), (Xo,Cf,YZdk),(Xo,Cf,YZdl),(Xo,Cf,YZdm),(Xo,Cf,YZdn),(Xo,Cf,YZdo),(Xo,Cf, YZdp),(Xo,Cf,YZdq),(Xo,Cf,YZdr),(Xo,Cf,YZds),(Xo,Cf,YZdt),(Xo,Cf,YZdu),(Xo,Cf, YZdv),(Xo,Cf,YZdw),(Xo,Cf,YZdx),(Xo,Cf,YZdy),(Xo,Cf,YZdz),(Xo,Cf,YZea),(Xo, Cf,YZeb),(Xo,Cf,YZec),(Xo,Cf,YZed),(Xo,Cf,YZee),(Xo,Cf,YZef),(Xo,Cf,YZeg),(Xo, Cf,YZeh),(Xo,Cf,YZei),(Xo,Cf,YZej),(Xo,Cf,YZek),(Xo,Cf,YZel),(Xo,Cf,YZem),(Xo, Cf,YZen),(Xo,Cf,YZeo),(Xo,Cf,YZep),(Xo,Cf,YZeq),(Xo,Cf,YZer),(Xo,Cf,YZes),(Xo, Cf,YZet),(Xo,Cf,YZeu),(Xo,Cf,YZev),(Xo,Cf,YZew),(Xo,Cf,YZex),(Xo,Cf,YZey), (Xo,Cf,YZez),(Xo,Cf,YZfa),(Xo,Cf,YZfb),(Xo,Cf,YZfc),(Xo,Cf,YZfd),(Xo,Cf,YZfe), (Xo,Cf,YZff),(Xo,Cf,YZfg),(Xo,Cf,YZfh),(Xo,Cf,YZfi),(Xo,Cf,YZfj),(Xo,Cf,YZfk),(Xo, Cf,YZfl),(Xo,Cf,YZfm),(Xo,Cf,YZfn),(Xo,Cf,YZfo),(Xo,Cf,YZfp),(Xo,Cf,YZfq),(Xo, Cf,YZfr),(Xo,Cf,YZfs),(Xo,Cf,YZft),(Xo,Cf,YZfu),(Xo,Cf,YZfv),(Xo,Cf,YZfw),(Xo, Cf,YZfx),(Xo,Cf,YZfy),(Xo,Cf,YZfz),(Xo,Cf,YZga),(Xo,Cf,YZgb),(Xo,Cf,YZgc), (Xo,Cf,YZgd),(Xo,Cf,YZge),(Xo,Cf,YZgf),(Xo,Cf,YZgg),(Xo,Cf,YZgh),(Xo,Cf,YZgi), (Xo,Cf,YZgj),(Xo,Cf,YZgk),(Xo,Cf,YZgl),(Xo,Cf,YZgm),(Xo,Cf,YZgn),(Xo,Cf,YZgo), (Xo,Cf,YZgp),(Xo,Cf,YZgq),(Xo,Cf,YZgr),(Xo,Cf,YZgs),(Xo,Cf,YZgt), (Xp,Ca,YZa),(Xp,Ca,YZb),(Xp,Ca,YZc),(Xp,Ca,YZd),(Xp,Ca,YZe),(Xp,Ca,YZf),(Xp, Ca,YZg),(Xp,Ca,YZh),(Xp,Ca,YZi),(Xp,Ca,YZj),(Xp,Ca,YZk),(Xp,Ca,YZl),(Xp,Ca,YZm), (Xp,Ca,YZn),(Xp,Ca,YZo),(Xp,Ca,YZp),(Xp,Ca,YZq),(Xp,Ca,YZr),(Xp,Ca,YZs), (Xp,Ca,YZt),(Xp,Ca,YZu),(Xp,Ca,YZv),(Xp,Ca,YZw),(Xp,Ca,YZx),(Xp,Ca,YZy),(Xp, Ca,YZz),(Xp,Ca,YZaa),(Xp,Ca,YZab),(Xp,Ca,YZac),(Xp,Ca,YZad),(Xp,Ca,YZae),(Xp, Ca,YZaf),(Xp,Ca,YZag),(Xp,Ca,YZah),(Xp,Ca,YZai),(Xp,Ca,YZaj),(Xp,Ca,YZak), (Xp,Ca,YZal),(Xp,Ca,YZam),(Xp,Ca,YZan),(Xp,Ca,YZao),(Xp,Ca,YZap),(Xp,Ca,YZaq), (Xp,Ca,YZar),(Xp,Ca,YZas),(Xp,Ca,YZat),(Xp,Ca,YZau),(Xp,Ca,YZav),(Xp,Ca, YZaw),(Xp,Ca,YZax),(Xp,Ca,YZay),(Xp,Ca,YZaz),(Xp,Ca,YZba),(Xp,Ca,YZbb),(Xp, Ca,YZbc),(Xp,Ca,YZbd),(Xp,Ca,YZbe),(Xp,Ca,YZbf),(Xp,Ca,YZbg),(Xp,Ca,YZbh),(Xp, Ca,YZbi),(Xp,Ca,YZbj),(Xp,Ca,YZbk),(Xp,Ca,YZbl),(Xp,Ca,YZbm),(Xp,Ca,YZbn), (Xp,Ca,YZbo),(Xp,Ca,YZbp),(Xp,Ca,YZbq),(Xp,Ca,YZbr),(Xp,Ca,YZbs),(Xp,Ca, YZbt),(Xp,Ca,YZbu),(Xp,Ca,YZbv),(Xp,Ca,YZbw),(Xp,Ca,YZbx),(Xp,Ca,YZby),(Xp, Ca,YZbz),(Xp,Ca,YZca),(Xp,Ca,YZcb),(Xp,Ca,YZcc),(Xp,Ca,YZcd),(Xp,Ca,YZce),(Xp, Ca,YZcf),(Xp,Ca,YZcg),(Xp,Ca,YZch),(Xp,Ca,YZci),(Xp,Ca,YZcj),(Xp,Ca,YZck), (Xp,Ca,YZcl),(Xp,Ca,YZcm),(Xp,Ca,YZcn),(Xp,Ca,YZco),(Xp,Ca,YZcp),(Xp,Ca,YZcq), (Xp,Ca,YZcr),(Xp,Ca,YZcs),(Xp,Ca,YZct),(Xp,Ca,YZcu),(Xp,Ca,YZcv),(Xp,Ca, YZcw),(Xp,Ca,YZcx),(Xp,Ca,YZcy),(Xp,Ca,YZcz),(Xp,Ca,YZda),(Xp,Ca,YZdb),(Xp, Ca,YZdc),(Xp,Ca,YZdd),(Xp,Ca,YZde),(Xp,Ca,YZdf),(Xp,Ca,YZdg),(Xp,Ca,YZdh),(Xp, Ca,YZdi),(Xp,Ca,YZdj),(Xp,Ca,YZdk),(Xp,Ca,YZdl),(Xp,Ca,YZdm),(Xp,Ca,YZdn), (Xp,Ca,YZdo),(Xp,Ca,YZdp),(Xp,Ca,YZdq),(Xp,Ca,YZdr),(Xp,Ca,YZds),(Xp,Ca, YZdt),(Xp,Ca,YZdu),(Xp,Ca,YZdv),(Xp,Ca,YZdw),(Xp,Ca,YZdx),(Xp,Ca,YZdy),(Xp, Ca,YZdz),(Xp,Ca,YZea),(Xp,Ca,YZeb),(Xp,Ca,YZec),(Xp,Ca,YZed),(Xp,Ca,YZee),(Xp, Ca,YZef),(Xp,Ca,YZeg),(Xp,Ca,YZeh),(Xp,Ca,YZei),(Xp,Ca,YZej),(Xp,Ca,YZek), (Xp,Ca,YZel),(Xp,Ca,YZem),(Xp,Ca,YZen),(Xp,Ca,YZeo),(Xp,Ca,YZep),(Xp,Ca,YZeq), (Xp,Ca,YZer),(Xp,Ca,YZes),(Xp,Ca,YZet),(Xp,Ca,YZeu),(Xp,Ca,YZev),(Xp,Ca, YZew),(Xp,Ca,YZex),(Xp,Ca,YZey),(Xp,Ca,YZez),(Xp,Ca,YZfa),(Xp,Ca,YZfb),(Xp, Ca,YZfc),(Xp,Ca,YZfd),(Xp,Ca,YZfe),(Xp,Ca,YZff),(Xp,Ca,YZfg),(Xp,Ca,YZfh),(Xp, Ca,YZfi),(Xp,Ca,YZfj),(Xp,Ca,YZfk),(Xp,Ca,YZfl),(Xp,Ca,YZfm),(Xp,Ca,YZfn),(Xp, Ca,YZfo),(Xp,Ca,YZfp),(Xp,Ca,YZfq),(Xp,Ca,YZfr),(Xp,Ca,YZfs),(Xp,Ca,YZft),(Xp, Ca,YZfu),(Xp,Ca,YZfv),(Xp,Ca,YZfw),(Xp,Ca,YZfx),(Xp,Ca,YZfy),(Xp,Ca,YZfz),(Xp, Ca,YZga),(Xp,Ca,YZgb),(Xp,Ca,YZgc),(Xp,Ca,YZgd),(Xp,Ca,YZge),(Xp,Ca,YZgf), (Xp,Ca,YZgg),(Xp,Ca,YZgh),(Xp,Ca,YZgi),(Xp,Ca,YZgj),(Xp,Ca,YZgk),(Xp,Ca,YZgl), (Xp,Ca,YZgm),(Xp,Ca,YZgn),(Xp,Ca,YZgo),(Xp,Ca,YZgp),(Xp,Ca,YZgq),(Xp, Ca,YZgr),(Xp,Ca,YZgs),(Xp,Ca,YZgt),(Xp,Cb,YZa),(Xp,Cb,YZb),(Xp,Cb,YZc),(Xp, Cb,YZd),(Xp,Cb,YZe),(Xp,Cb,YZf),(Xp,Cb,YZg),(Xp,Cb,YZh),(Xp,Cb,YZi),(Xp,Cb, YZj),(Xp,Cb,YZk),(Xp,Cb,YZl),(Xp,Cb,YZm),(Xp,Cb,YZn),(Xp,Cb,YZo),(Xp,Cb,YZp), (Xp,Cb,YZq),(Xp,Cb,YZr),(Xp,Cb,YZs),(Xp,Cb,YZt),(Xp,Cb,YZu),(Xp,Cb,YZv),(Xp, Cb,YZw),(Xp,Cb,YZx),(Xp,Cb,YZy),(Xp,Cb,YZz),(Xp,Cb,YZaa),(Xp,Cb,YZab),(Xp, Cb,YZac),(Xp,Cb,YZad),(Xp,Cb,YZae),(Xp,Cb,YZaf),(Xp,Cb,YZag),(Xp,Cb,YZah), (Xp,Cb,YZai),(Xp,Cb,YZaj),(Xp,Cb,YZak),(Xp,Cb,YZal),(Xp,Cb,YZam),(Xp,Cb, YZan),(Xp,Cb,YZao),(Xp,Cb,YZap),(Xp,Cb,YZaq),(Xp,Cb,YZar),(Xp,Cb,YZas),(Xp, Cb,YZat),(Xp,Cb,YZau),(Xp,Cb,YZav),(Xp,Cb,YZaw),(Xp,Cb,YZax),(Xp,Cb,YZay), (Xp,Cb,YZaz),(Xp,Cb,YZba),(Xp,Cb,YZbb),(Xp,Cb,YZbc),(Xp,Cb,YZbd),(Xp,Cb,YZbe), (Xp,Cb,YZbf),(Xp,Cb,YZbg),(Xp,Cb,YZbh),(Xp,Cb,YZbi),(Xp,Cb,YZbj),(Xp,Cb, YZbk),(Xp,Cb,YZbl),(Xp,Cb,YZbm),(Xp,Cb,YZbn),(Xp,Cb,YZbo),(Xp,Cb,YZbp),(Xp, Cb,YZbq),(Xp,Cb,YZbr),(Xp,Cb,YZbs),(Xp,Cb,YZbt),(Xp,Cb,YZbu),(Xp,Cb,YZbv), (Xp,Cb,YZbw),(Xp,Cb,YZbx),(Xp,Cb,YZby),(Xp,Cb,YZbz),(Xp,Cb,YZca),(Xp,Cb, YZcb),(Xp,Cb,YZcc),(Xp,Cb,YZcd),(Xp,Cb,YZce),(Xp,Cb,YZcf),(Xp,Cb,YZcg),(Xp, Cb,YZch),(Xp,Cb,YZci),(Xp,Cb,YZcj),(Xp,Cb,YZck),(Xp,Cb,YZcl),(Xp,Cb,YZcm),(Xp, Cb,YZcn),(Xp,Cb,YZco),(Xp,Cb,YZcp),(Xp,Cb,YZcq),(Xp,Cb,YZcr),(Xp,Cb,YZcs), (Xp,Cb,YZct),(Xp,Cb,YZcu),(Xp,Cb,YZcv),(Xp,Cb,YZcw),(Xp,Cb,YZcx),(Xp,Cb, YZcy),(Xp,Cb,YZcz),(Xp,Cb,YZda),(Xp,Cb,YZdb),(Xp,Cb,Y Zdc),(Xp,Cb,YZdd),(Xp, Cb,YZde),(Xp,Cb,YZdf),(Xp,Cb,YZdg),(Xp,Cb,YZdh),(Xp,Cb,YZdi),(Xp,Cb,YZdj),(Xp, Cb,YZdk),(Xp,Cb,YZdl),(Xp,Cb,YZdm),(Xp,Cb,YZdn),(Xp,Cb,YZdo),(Xp,Cb,YZdp), (Xp,Cb,YZdq),(Xp,Cb,YZdr),(Xp,Cb,YZds),(Xp,Cb,YZdt),(Xp,Cb,YZdu),(Xp,Cb, YZdv),(Xp,Cb,YZdw),(Xp,Cb,YZdx),(Xp,Cb,YZdy),(Xp,Cb,YZdz),(Xp,Cb,YZea),(Xp, Cb,YZeb),(Xp,Cb,YZec),(Xp,Cb,YZed),(Xp,Cb,YZee),(Xp,Cb,YZef),(Xp,Cb,YZeg), (Xp,Cb,YZeh),(Xp,Cb,YZei),(Xp,Cb,YZej),(Xp,Cb,YZek),(Xp,Cb,YZel),(Xp,Cb,YZem), (Xp,Cb,YZen),(Xp,Cb,YZeo),(Xp,Cb,YZep),(Xp,Cb,YZeq),(Xp,Cb,YZer),(Xp,Cb, YZes),(Xp,Cb,YZet),(Xp,Cb,YZeu),(Xp,Cb,YZev),(Xp,Cb,YZew),(Xp,Cb,YZex),(Xp, Cb,YZey),(Xp,Cb,YZez),(Xp,Cb,YZfa),(Xp,Cb,YZfb),(Xp,Cb,YZfc),(Xp,Cb,YZfd),(Xp, Cb,YZfe),(Xp,Cb,YZff),(Xp,Cb,YZfg),(Xp,Cb,YZfh),(Xp,Cb,YZfi),(Xp,Cb,YZfj), (Xp,Cb,YZfk),(Xp,Cb,YZfl),(Xp,Cb,YZfm),(Xp,Cb,YZfn),(Xp,Cb,YZfo),(Xp,Cb,YZfp), (Xp,Cb,YZfq),(Xp,Cb,YZfr),(Xp,Cb,YZfs),(Xp,Cb,YZft),(Xp,Cb,YZfu),(Xp,Cb,YZfv), (Xp,Cb,YZfw),(Xp,Cb,YZfx),(Xp,Cb,YZfy),(Xp,Cb,YZfz),(Xp,Cb,YZga),(Xp,Cb, YZgb),(Xp,Cb,YZgc),(Xp,Cb,YZgd),(Xp,Cb,YZge),(Xp,Cb,YZgf),(Xp,Cb,YZgg),(Xp, Cb,YZgh),(Xp,Cb,YZgi),(Xp,Cb,YZgj),(Xp,Cb,YZgk),(Xp,Cb,YZgl),(Xp,Cb,YZgm), (Xp,Cb,YZgn),(Xp,Cb,YZgo),(Xp,Cb,YZgp),(Xp,Cb,YZgq),(Xp,Cb,YZgr),(Xp,Cb,YZgs), (Xp,Cb,YZgt),(Xp,Cc,YZa),(Xp,Cc,YZb),(Xp,Cc,YZc),(Xp,Cc,YZd),(Xp,Cc,YZe), (Xp,Cc,YZf),(Xp,Cc,YZg),(Xp,Cc,YZh),(Xp,Cc,YZi),(Xp,Cc,YZj),(Xp,Cc,YZk),(Xp, Cc,YZl),(Xp,Cc,YZm),(Xp,Cc,YZn),(Xp,Cc,YZo),(Xp,Cc,YZp),(Xp,Cc,YZq),(Xp,Cc, YZr),(Xp,Cc,YZs),(Xp,Cc,YZt),(Xp,Cc,YZu),(Xp,Cc,YZv),(Xp,Cc,YZw),(Xp,Cc,YZx), (Xp,Cc,YZy),(Xp,Cc,YZz),(Xp,Cc,YZaa),(Xp,Cc,YZab),(Xp,Cc,YZac),(Xp,Cc,YZad), (Xp,Cc,YZae),(Xp,Cc,YZaf),(Xp,Cc,YZag),(Xp,Cc,YZah),(Xp,Cc,YZai),(Xp,Cc, YZaj),(Xp,Cc,YZak),(Xp,Cc,YZal),(Xp,Cc,YZam),(Xp,Cc,YZan),(Xp,Cc,YZao),(Xp, Cc,YZap),(Xp,Cc,YZaq),(Xp,Cc,YZar),(Xp,Cc,YZas),(Xp,Cc,YZat),(Xp,Cc,YZau),(Xp, Cc,YZav),(Xp,Cc,YZaw),(Xp,Cc,YZax),(Xp,Cc,YZay),(Xp,Cc,YZaz),(Xp,Cc,YZba), (Xp,Cc,YZbb),(Xp,Cc,YZbc),(Xp,Cc,YZbd),(Xp,Cc,YZbe),(Xp,Cc,YZbf),(Xp,Cc,YZbg), (Xp,Cc,YZbh),(Xp,Cc,YZbi),(Xp,Cc,YZbj),(Xp,Cc,YZbk),(Xp,Cc,YZbl),(Xp,Cc, YZbm),(Xp,Cc,YZbn),(Xp,Cc,YZbo),(Xp,Cc,YZbp),(Xp,Cc,YZbq),(Xp,Cc,YZbr),(Xp, Cc,YZbs),(Xp,Cc,YZbt),(Xp,Cc,YZbu),(Xp ,Cc,YZbv),(Xp,Cc,YZbw),(Xp,Cc,YZbx), (Xp,Cc,YZby),(Xp,Cc,YZbz),(Xp,Cc,YZca),(Xp,Cc,YZcb),(Xp,Cc,YZcc),(Xp,Cc,YZcd), (Xp,Cc,YZce),(Xp,Cc,YZcf),(Xp,Cc,YZcg),(Xp,Cc,YZch),(Xp,Cc,YZci),(Xp,Cc, YZcj),(Xp,Cc,YZck),(Xp,Cc,YZcl),(Xp,Cc,YZcm),(Xp,Cc,YZcn),(Xp,Cc,YZco),(Xp, Cc,YZcp),(Xp,Cc,YZcq),(Xp,Cc,YZcr),(Xp,Cc,YZcs),(Xp,Cc,YZct),(Xp,Cc,YZcu),(Xp, Cc,YZcv),(Xp,Cc,YZcw),(Xp,Cc,YZcx),(Xp,Cc,YZcy),(Xp,Cc,YZcz),(Xp,Cc,YZda), (Xp,Cc,YZdb),(Xp,Cc,YZdc),(Xp,Cc,YZdd),(Xp,Cc,YZde),(Xp,Cc,YZdf),(Xp,Cc,YZdg), (Xp,Cc,YZdh),(Xp,Cc,YZdi),(Xp,Cc,YZdj),(Xp,Cc,YZdk),(Xp,Cc,YZdl),(Xp,Cc, YZdm),(Xp,Cc,YZdn),(Xp,Cc,YZdo),(Xp,Cc,YZdp),(Xp,Cc,YZdq),(Xp,Cc,YZdr),(Xp, Cc,YZds),(Xp,Cc,YZdt),(Xp,Cc,YZdu),(Xp,Cc,YZdv),(Xp,Cc,YZdw),(Xp,Cc,YZdx), (Xp,Cc,YZdy),(Xp,Cc,YZdz),(Xp,Cc,YZea),(Xp,Cc,YZeb),(Xp,Cc,YZec),(Xp,Cc,YZed), (Xp,Cc,YZee),(Xp,Cc,YZef),(Xp,Cc,YZeg),(Xp,Cc,YZeh),(Xp,Cc,YZei),(Xp,Cc, YZej),(Xp,Cc,YZek),(Xp,Cc,YZel),(Xp,Cc,YZem),(Xp,Cc,YZen),(Xp,Cc,YZeo),(Xp, Cc,YZep),(Xp,Cc,YZeq),(Xp,Cc,YZer),(Xp,Cc,YZes),(Xp,Cc,YZet),(Xp,Cc,YZeu),(Xp, Cc,YZev),(Xp,Cc,YZew),(Xp,Cc,YZex),(Xp,Cc,YZey),(Xp,Cc,YZez),(Xp,Cc,YZfa), (Xp,Cc,YZfb),(Xp,Cc,YZfc),(Xp,Cc,YZfd),(Xp,Cc,YZfe),(Xp,Cc,YZff),(Xp,Cc,YZfg), (Xp,Cc,YZfh),(Xp,Cc,YZfi),(Xp,Cc,YZfj),(Xp,Cc,YZfk),(Xp,Cc,YZfl),(Xp,Cc,YZfm), (Xp,Cc,YZfn),(Xp,Cc,YZfo),(Xp,Cc,YZfp),(Xp,Cc,YZfq),(Xp,Cc,YZfr),(Xp,Cc,YZfs), (Xp,Cc,YZft),(Xp,Cc,YZfu),(Xp,Cc,YZfv),(Xp,Cc,YZfw),(Xp,Cc,YZfx),(Xp,Cc, YZfy),(Xp,Cc,YZfz),(Xp,Cc,YZga),(Xp,Cc,YZgb),(Xp,Cc,YZgc),(Xp,Cc,YZgd),(Xp, Cc,YZge),(Xp,Cc,YZgf),(Xp,Cc,YZgg),(Xp,Cc,YZgh),(Xp,Cc,YZgi),(Xp,Cc,YZgj),(Xp, Cc,YZgk),(Xp,Cc,YZgl),(Xp,Cc,YZgm),(Xp,Cc,YZgn),(Xp,Cc,YZgo),(Xp,Cc,YZgp), (Xp,Cc,YZgq),(Xp,Cc,YZgr),(Xp,Cc,YZgs),(Xp,Cc,YZgt),(Xp,Cd,YZa),(Xp,Cd,YZb), (Xp,Cd,YZc),(Xp,Cd,YZd),(Xp,Cd,YZe),(Xp,Cd,YZf),(Xp,Cd,YZg),(Xp,Cd,YZh), (Np,Cd,YZi),(Xp,Cd,YZj),(Xp,Cd,YZk),(Xp,Cd,YZl),(Xp,Cd,YZm),(Xp,Cd,YZn),(Xp, Cd,YZo),(Xp,Cd,YZp),(Xp,Cd,YZq),(Xp,Cd,YZr),(Xp,Cd,YZs),(Xp,Cd,YZt),(Xp,Cd, YZu),(Xp,Cd,YZv),(Xp,Cd,YZw),(Xp,Cd,YZx),(Xp,Cd,YZy),(Xp,Cd,YZz),(Xp,Cd, YZaa),(Xp,Cd,YZab),(Xp,Cd,YZac),(Xp,Cd,YZad),(Xp,Cd,YZae),(Xp,Cd,YZaf),(Xp, Cd,YZag),(Xp,Cd,YZah),(Xp,Cd,YZai),(Xp,Cd,YZaj),(Xp,Cd,YZak),(Xp,Cd,YZal),(Xp, Cd,YZam),(Xp,Cd,YZan),(Xp,Cd,YZao),(Xp,Cd,YZap),(Xp,Cd,YZaq),(Xp,Cd,YZar), (Xp,Cd,YZas),(Xp,Cd,YZat),(Xp,Cd,YZau),(Xp,Cd,YZav),(Xp,Cd,YZaw),(Xp,Cd, YZax),(Xp,Cd,YZay),(Xp,Cd,YZaz),(Xp,Cd,YZba),(Xp,Cd,YZbb),(Xp,Cd,YZbc),(Xp, Cd,YZbd),(Xp,Cd,YZbe),(Xp,Cd,YZbf),(Xp,Cd,YZbg),(Xp,Cd,YZbh),(Xp,Cd,YZbi), (Xp,Cd,YZbj),(Xp,Cd,YZbk),(Xp,Cd,YZbl),(Xp,Cd,YZbm),(Xp,Cd,YZbn),(Xp,Cd,YZbo), (Xp,Cd,YZbp),(Xp,Cd,YZbq),(Xp,Cd,YZbr),(Xp,Cd,YZbs),(Xp,Cd,YZbt),(Xp,Cd, YZbu),(Xp,Cd,YZbv),(Xp,Cd,YZbw),(Xp,Cd,YZbx),(Xp,Cd,YZby),(Xp,Cd,YZbz),(Xp, Cd,YZca),(Xp,Cd,YZcb),(Xp,Cd,YZcc),(Xp,Cd,YZcd),(Xp,Cd,YZce),(Xp,Cd,YZcf), (Xp,Cd,YZcg),(Xp,Cd,YZch),(Xp,Cd,YZci),(Xp,Cd,YZcj),(Xp,Cd,YZck),(Xp,Cd,YZcl), (Xp,Cd,YZcm),(Xp,Cd,YZcn),(Xp,Cd,YZco),(Xp,Cd,YZcp),(Xp,Cd,YZcq),(Xp, Cd,YZcr),(Xp,Cd,YZcs),(Xp,Cd,YZct),(Xp,Cd,YZcu),(Xp,Cd,YZcv),(Xp,Cd,YZcw),(Xp, Cd,YZcx),(Xp,Cd,YZcy),(Xp,Cd,YZcz),(Xp,Cd,YZda),(Xp,Cd,YZdb),(Xp,Cd,YZdc), (Xp,Cd,YZdd),(Xp,Cd,YZde),(Xp,Cd,YZdf),(Xp,Cd,YZdg),(Xp,Cd,YZdh),(Xp,Cd, YZdi),(Xp,Cd,YZdj),(Xp,Cd,YZdk),(Xp,Cd,YZdl),(Xp,Cd,YZdm),(Xp,Cd,YZdn),(Xp, Cd,YZdo),(Xp,Cd,YZdp),(Xp,Cd,YZdq),(Xp,Cd,YZdr),(Xp,Cd,YZds),(Xp,Cd,YZdt), (Xp,Cd,YZdu),(Xp,Cd,YZdv),(Xp,Cd,YZdw),(Xp,Cd,YZdx),(Xp,Cd,YZdy),(Xp,Cd, YZdz),(Xp,Cd,YZea),(Xp,Cd,YZeb),(Xp,Cd,YZec),(Xp,Cd,YZed),(Xp,Cd,YZee),(Xp, Cd,YZef),(Xp,Cd,YZeg),(Xp,Cd,YZeh),(Xp,Cd,YZei),(Xp,Cd,YZej),(Xp,Cd,YZek),(Xp, Cd,YZel),(Xp,Cd,YZem),(Xp,Cd,YZen),(Xp,Cd,YZeo),(Xp,Cd,YZep),(Xp,Cd,YZeq), (Xp,Cd,YZer),(Xp,Cd,YZes),(Xp,Cd,YZet),(Xp,Cd,YZeu),(Xp,Cd,YZev),(Xp,Cd, YZew),(Xp,Cd,YZex),(Xp,Cd,YZey),(Xp,Cd,YZez),(Xp,Cd,YZfa),(Xp,Cd,YZfb),(Xp, Cd,YZfc),(Xp,Cd,YZfd),(Xp,Cd,YZfe),(Xp,Cd,YZff),(Xp,Cd,YZfg),(Xp,Cd,YZfh),(Xp, Cd,YZfi),(Xp,Cd,YZfj),(Xp,Cd,YZfk),(Xp,Cd,YZfl),(Xp,Cd,YZfm),(Xp,Cd,YZfn),(Xp, Cd,YZfo),(Xp,Cd,YZfp),(Xp,Cd,YZfq),(Xp,Cd,YZfr),(Xp,Cd,YZfs),(Xp,Cd,YZft), (Xp,Cd,YZfu),(Xp,Cd,YZfv),(Xp,Cd,YZfw),(Xp,Cd,YZfx),(Xp,Cd,YZfy),(Xp,Cd,YZfz), (Xp,Cd,YZga),(Xp,Cd,YZgb),(Xp,Cd,YZgc),(Xp,Cd,YZgd),(Xp,Cd,YZge),(Xp,Cd, YZgf),(Xp,Cd,YZgg),(Xp,Cd,YZgh),(Xp,Cd,YZgi),(Xp,Cd,YZgj),(Xp,Cd,YZgk),(Xp, Cd,YZgl),(Xp,Cd,YZgm),(Xp,Cd,YZgn),(Xp,Cd,YZgo),(Xp,Cd,YZgp),(Xp,Cd,YZgq), (Xp,Cd,YZgr),(Xp,Cd,YZgs),(Xp,Cd,YZgt),(Xp,Ce,YZa),(Xp,Ce,YZb),(Xp,Ce,YZc), (Xp,Ce,YZd),(Xp,Ce,YZe),(Xp,Ce,YZf),(Xp,Ce,YZg),(Xp,Ce,YZh),(Xp,Ce,YZi),(Xp, Ce,YZj),(Xp,Ce,YZk),(Xp,Ce,YZl),(Xp,Ce,YZm),(Xp,Ce,YZn),(Xp,Ce,YZo),(Xp,Ce, YZp),(Xp,Ce,YZq),(Xp,Ce,YZr),(Xp,Ce,YZs),(Xp,Ce,YZt),(Xp,Ce,YZu),(Xp,Ce,YZv), (Xp,Ce,YZw),(Xp,Ce,YZx),(Xp,Ce,YZy),(Xp,Ce,YZz),(Xp,Ce,YZaa),(Xp,Ce,YZab), (Xp,Ce,YZac),(Xp,Ce,YZad),(Xp,Ce,YZae),(Xp,Ce,YZaf),(Xp,Ce,YZag),(Xp,Ce,YZah), (Xp,Ce,YZai),(Xp,Ce,YZaj),(Xp,Ce,YZak),(Xp,Ce,YZal),(Xp,Ce,YZam),(Xp,Ce, YZan),(Xp,Ce,YZao),(Xp,Ce,YZap),(Xp,Ce,YZaq),(Xp,Ce,YZar),(Xp,Ce,YZas),(Xp, Ce,YZat),(Xp,Ce,YZau),(Xp,Ce,YZav),(Xp,Ce,YZaw),(Xp,Ce,YZax),(Xp,Ce,YZay),(Xp, Ce,YZaz),(Xp,Ce,YZba),(Xp,Ce,YZbb),(Xp,Ce,YZbc),(Xp,Ce,YZbd),(Xp,Ce,YZbe), (Xp,Ce,YZbf),(Xp,Ce,YZbg),(Xp,Ce,YZbh),(Xp,Ce,YZbi),(Xp,Ce,YZbj),(Xp,Ce,YZbk), (Xp,Ce,YZbl),(Xp,Ce,YZbm),(Xp,Ce,YZbn),(Xp,Ce,YZbo),(Xp,Ce,YZbp),(Xp, Ce,YZbq),(Xp,Ce,YZbr),(Xp,Ce,YZbs),(Xp,Ce,YZbt),(Xp,Ce,YZbu),(Xp,Ce,YZbv),(Xp, Ce,YZbw),(Xp,Ce,YZbx),(Xp,Ce,YZby),(Xp,Ce,YZbz),(Xp,Ce,YZca),(Xp,Ce,YZcb), (Xp,Ce,YZcc),(Xp,Ce,YZcd),(Xp,Ce,YZce),(Xp,Ce,YZcf),(Xp,Ce,YZcg),(Xp,Ce, YZch),(Xp,Ce,YZci),(Xp,Ce,YZcj),(Xp,Ce,YZck),(Xp,Ce,YZcl),(Xp,Ce,YZcm),(Xp, Ce,YZcn),(Xp,Ce,YZco),(Xp,Ce,YZcp),(Xp,Ce,YZcq),(Xp,Ce,YZcr),(Xp,Ce,YZcs),(Xp, Ce,YZct),(Xp,Ce,YZcu),(Xp,Ce,YZcv),(Xp,Ce,YZcw),(Xp,Ce,YZcx),(Xp,Ce,YZcy), (Xp,Ce,YZcz),(Xp,Ce,YZda),(Xp,Ce,YZdb),(Xp,Ce,YZdc),(Xp,Ce,YZdd),(Xp,Ce, YZde),(Xp,Ce,YZdf),(Xp,Ce,YZdg),(Xp,Ce,YZdh),(Xp,Ce,YZdi),(Xp,Ce,YZdj),(Xp, Ce,YZdk),(Xp,Ce,YZdl),(Xp,Ce,YZdm),(Xp,Ce,YZdn),(Xp,Ce,YZdo),(Xp,Ce,YZdp), (Xp,Ce,YZdq),(Xp,Ce,YZdr),(Xp,Ce,YZds),(Xp,Ce,YZdt),(Xp,Ce,YZdu),(Xp,Ce,YZdv), (Xp,Ce,YZdw),(Xp,Ce,YZdx),(Xp,Ce,YZdy),(Xp,Ce,YZdz),(Xp,Ce,YZea),(Xp,Ce, YZeb),(Xp,Ce,YZec),(Xp,Ce,YZed),(Xp,Ce,YZee),(Xp,Ce,YZef),(Xp,Ce,YZeg),(Xp, Ce,YZeh),(Xp,Ce,YZei),(Xp,Ce,YZej),(Xp,Ce,YZek),(Xp,Ce,YZel),(Xp,Ce,YZem),(Xp, Ce,YZen),(Xp,Ce,YZeo),(Xp,Ce,YZep),(Xp,Ce,YZeq),(Xp,Ce,YZer),(Xp,Ce,YZes), (Xp,Ce,YZet),(Xp,Ce,YZeu),(Xp,Ce,YZev),(Xp,Ce,YZew),(Xp,Ce,YZex),(Xp,Ce,YZey), (Xp,Ce,YZez),(Xp,Ce,YZfa),(Xp,Ce,YZfb),(Xp,Ce,YZfe),(Xp,Ce,YZfd),(Xp,Ce, YZfe),(Xp,Ce,YZff),(Xp,Ce,YZfg),(Xp,Ce,YZfh),(Xp,Ce,YZfi),(Xp,Ce,YZfj),(Xp,Ce, YZfk),(Xp,Ce,YZfl),(Xp,Ce,YZfm),(Xp,Ce,YZfn),(Xp,Ce,YZfo),(Xp,Ce,YZfp),(Xp,Ce, YZfq),(Xp,Ce,YZfr),(Xp,Ce,YZfs),(Xp,Ce,YZft),(Xp,Ce,YZfu),(Xp,Ce,YZfv),(Xp,Ce, YZfw),(Xp,Ce,YZfx),(Xp,Ce,YZfy),(Xp,Ce,YZfz),(Xp,Ce,YZga),(Xp,Ce,YZgb),(Xp, Ce,YZgc),(Xp,Ce,YZgd),(Xp,Ce,YZge),(Xp,Ce,YZgf),(Xp,Ce,YZgg),(Xp,Ce,YZgh), (Xp,Ce,YZgi),(Xp,Ce,YZgj),(Xp,Ce,YZgk),(Xp,Ce,YZgl),(Xp,Ce,YZgm),(Xp,Ce,YZgn), (Xp,Ce,YZgo),(Xp,Ce,YZgp),(Xp,Ce,YZgq),(Xp,Ce,YZgr),(Xp,Ce,YZgs),(Xp,Ce, YZgt),(Xp,Cf,YZa),(Xp,Cf,YZb),(Xp,Cf,YZc),(Xp,Cf,YZd),(Xp,Cf,YZe),(Xp,Cf,YZf), (Xp,Cf,YZg),(Xp,Cf,YZh),(Xp,Cf,YZi),(Xp,Cf,YZj),(Xp,Cf,YZk),(Xp,Cf,YZl),(Xp,Cf, YZm),(Xp,Cf,YZn),(Xp,Cf,YZo),(Xp,Cf,YZp),(Xp,Cf,YZq),(Xp,Cf,YZr),(Xp,Cf,YZs), (Xp,Cf,YZt),(Xp,Cf,YZu),(Xp,Cf,YZv),(Xp,Cf,YZw),(Xp,Cf,YZx),(Xp,Cf,YZy),(Xp, Cf,YZz),(Xp,Cf,YZaa),(Xp,Cf,YZab),(Xp,Cf,YZac),(Xp,Cf,YZad),(Xp,Cf,YZae),(Xp, Cf,YZaf),(Xp,Cf,YZag),(Xp,Cf,YZah),(Xp,Cf,YZai),(Xp,Cf,YZaj),(Xp,Cf,YZak),(Xp, Cf,YZal),(Xp,Cf,YZam),(Xp,Cf,YZan),(Xp,Cf,YZao),(Xp,Cf,YZap),(Xp,Cf,YZaq),(Xp, Cf,YZar),(Xp,Cf,YZas),(Xp,Cf,YZat),(Xp,Cf,YZau),(Xp,Cf,YZav),(Xp,Cf,YZaw), (Xp,Cf,YZax),(Xp,Cf,YZay),(Xp,Cf,YZaz),(Xp,Cf,YZba),(Xp,Cf,YZbb),(Xp,Cf,YZbc), (Xp,Cf,YZbd),(Xp,Cf,YZbe),(Xp,Cf,YZbf),(Xp,Cf,YZbg),(Xp,Cf,YZbh),(Xp,Cf,YZbi), (Xp,Cf,YZbj),(Xp,Cf,YZbk),(Xp,Cf,YZbl),(Xp,Cf,YZbm),(Xp,Cf,YZbn),(Xp,Cf,YZbo), (Xp,Cf,YZbp),(Xp,Cf,YZbq),(Xp,Cf,YZbr),(Xp,Cf,YZbs),(Xp,Cf,YZbt),(Xp,Cf, YZbu),(Xp,Cf,YZbv),(Xp,Cf,YZbw),(Xp,Cf,YZbx),(Xp,Cf,YZby),(Xp,Cf,YZbz),(Xp, Cf,YZca),(Xp,Cf,YZcb),(Xp,Cf,YZcc),(Xp,Cf,YZcd),(Xp,Cf,YZce),(Xp,Cf,YZcf),(Xp, Cf,YZcg),(Xp,Cf,YZch),(Xp,Cf,YZci),(Xp,Cf,YZcj),(Xp,Cf,YZck),(Xp,Cf,YZcl),(Xp, Cf,YZcm),(Xp,Cf,YZcn),(Xp,Cf,YZco),(Xp,Cf,YZcp),(Xp,Cf,YZcq),(Xp,Cf,YZcr),(Xp, Cf,YZcs),(Xp,Cf,YZct),(Xp,Cf,YZcu),(Xp,Cf,YZcv),(Xp,Cf,YZcw),(Xp,Cf,YZcx), (Xp,Cf,YZcy),(Xp,Cf,YZcz),(Xp,Cf,YZda),(Xp,Cf,YZdb),(Xp,Cf,YZdc),(Xp,Cf,YZdd), (Xp,Cf,YZde),(Xp,Cf,YZdf),(Xp,Cf,YZdg),(Xp,Cf,YZdh),(Xp,Cf,YZdi),(Xp,Cf,YZdj), (Xp,Cf,YZdk),(Xp,Cf,YZdl),(Xp,Cf,YZdm),(Xp,Cf,YZdn),(Xp,Cf,YZdo),(Xp,Cf, YZdp),(Xp,Cf,YZdq),(Xp,Cf,YZdr),(Xp,Cf,YZds),(Xp,Cf,YZdt),(Xp,Cf,YZdu),(Xp,Cf, YZdv),(Xp,Cf,YZdw),(Xp,Cf,YZdx),(Xp,Cf,YZdy),(Xp,Cf,YZdz),(Xp,Cf,YZea),(Xp, Cf,YZeb),(Xp,Cf,YZec),(Xp,Cf,YZed),(Xp,Cf,YZee),(Xp,Cf,YZef),(Xp,Cf,YZeg),(Xp, Cf,YZeh),(Xp,Cf,YZei),(Xp,Cf,YZej),(Xp,Cf,YZek),(Xp,Cf,YZel),(Xp,Cf,YZem),(Xp, Cf,YZen),(Xp,Cf,YZeo),(Xp,Cf,YZep),(Xp,Cf,YZeq),(Xp,Cf,YZer),(Xp,Cf,YZes),(Xp, Cf,YZet),(Xp,Cf,YZeu),(Xp,Cf,YZev),(Xp,Cf,YZew),(Xp,Cf,YZex),(Xp,Cf,YZey), (Xp,Cf,YZez),(Xp,Cf,YZfa),(Xp,Cf,YZfb),(Xp,Cf,YZfc),(Xp,Cf,YZfd),(Xp,Cf,YZfe), (Xp,Cf,YZff),(Xp,Cf,YZfg),(Xp,Cf,YZfh),(Xp,Cf,YZfi),(Xp,Cf,YZfj),(Xp,Cf,YZfk),(Xp, Cf,YZfl),(Xp,Cf,YZfm),(Xp,Cf,YZfn),(Xp,Cf,YZfo),(Xp,Cf,YZfp),(Xp,Cf,YZfq),(Xp, Cf,YZfr),(Xp,Cf,YZfs),(Xp,Cf,YZft),(Xp,Cf,YZfu),(Xp,Cf,YZfv),(Xp,Cf,YZfw),(Xp, Cf,YZfx),(Xp,Cf,YZfy),(Xp,Cf,YZfz),(Xp,Cf,YZga),(Xp,Cf,YZgb),(Xp,Cf,YZgc), (Xp,Cf,YZgd),(Xp,Cf,YZge),(Xp,Cf,YZgf),(Xp,Cf,YZgg),(Xp,Cf,YZgh),(Xp,Cf,YZgi), (Xp,Cf,YZgj),(Xp,Cf,YZgk),(Xp,Cf,YZgl),(Xp,Cf,YZgm),(Xp,Cf,YZgn),(Xp,Cf,YZgo), (Xp,Cf,YZgp),(Xp,Cf,YZgq),(Xp,Cf,YZgr),(Xp,Cf,YZgs),(Xp,Cf,YZgt), (Xq,Ca,YZa),(Xq,Ca,YZb),(Xq,Ca,YZc),(Xq,Ca,YZd),(Xq,Ca,YZe),(Xq,Ca,YZf),(Xq, Ca,YZg),(Xq,Ca,YZh),(Xq,Ca,YZi),(Xq,Ca,YZj),(Xq,Ca,YZk),(Xq,Ca,YZl),(Xq,Ca,YZm), (Xq,Ca,YZn),(Xq,Ca,YZo),(Xq,Ca,YZp),(Xq,Ca,YZq),(Xq,Ca,YZr),(Xq,Ca,YZs), (Xq,Ca,YZt),(Xq,Ca,YZu),(Xq,Ca,YZv),(Xq,Ca,YZw),(Xq,Ca,YZx),(Xq,Ca,YZy),(Xq, Ca,YZz),(Xq,Ca,YZaa),(Xq,Ca,YZab),(Xq,Ca,YZac),(Xq,Ca,YZad),(Xq,Ca,YZae),(Xq, Ca,YZaf),(Xq,Ca,YZag),(Xq,Ca,YZah),(Xq,Ca,YZai),(Xq,Ca,YZaj),(Xq,Ca,YZak), (Xq,Ca,YZal),(Xq,Ca,YZam),(Xq,Ca,YZan),(Xq,Ca,YZao),(Xq,Ca,YZap),(Xq,Ca,YZaq), (Xq,Ca,YZar),(Xq,Ca,YZas),(Xq,Ca,YZat),(Xq,Ca,YZau),(Xq,Ca,YZav),(Xq,Ca, YZaw),(Xq,Ca,YZax),(Xq,Ca,YZay),(Xq,Ca,YZaz),(Xq,Ca,YZba),(Xq,Ca,YZbb),(Xq, Ca,YZbc),(Xq,Ca,YZbd),(Xq,Ca,YZbe),(Xq,Ca,YZbf),(Xq,Ca,YZbg),(Xq,Ca,YZbh),(Xq, Ca,YZbi),(Xq,Ca,YZbj),(Xq,Ca,YZbk),(Xq,Ca,YZbl),(Xq,Ca,YZbm),(Xq,Ca,YZbn), (Xq,Ca,YZbo),(Xq,Ca,YZbp),(Xq,Ca,YZbq),(Xq,Ca,YZbr),(Xq,Ca,YZbs),(Xq,Ca, YZbt),(Xq,Ca,YZbu),(Xq,Ca,YZbv),(Xq,Ca,YZbw),(Xq,Ca,YZbx),(Xq,Ca,YZby),(Xq, Ca,YZbz),(Xq,Ca,YZca),(Xq,Ca,YZcb),(Xq,Ca,YZcc),(Xq,Ca,YZcd),(Xq,Ca,YZce),(Xq, Xq,Ca,YZcf),(Xq,Ca,YZcg),(Xq,Ca,YZch),(Xq,Ca,YZci),(Xq,Ca,YZcj),(Xq,Ca,YZck), (Xq,Ca,YZcl),(Xq,Ca,YZcm),(Xq,Ca,YZen),(Xq,Ca,YZco),(Xq,Ca,YZcp),(Xq,Ca,YZcq), (Xq,Ca,YZcr),(Xq,Ca,YZcs),(Xq,Ca,YZct),(Xq,Ca,YZcu),(Xq,Ca,YZcv),(Xq,Ca, YZcw),(Xq,Ca,YZcx),(Xq,Ca,YZey),(Xq,Ca,YZcz),(Xq,Ca,YZda),(Xq,Ca,YZdb),(Xq, Ca,YZdc),(Xq,Ca,YZdd),(Xq,Ca,YZde),(Xq,Ca,YZdf),(Xq,Ca,YZdg),(Xq,Ca,YZdh),(Xq, Ca,YZdi),(Xq,Ca,YZdj),(Xq,Ca,YZdk),(Xq,Ca,YZdl),(Xq,Ca,YZdm),(Xq,Ca,YZdn), (Xq,Ca,YZdo),(Xq,Ca,YZdp),(Xq,Ca,YZdq),(Xq,Ca,YZdr),(Xq,Ca,YZds),(Xq,Ca, YZdt),(Xq,Ca,YZdu),(Xq,Ca,YZdv),(Xq,Ca,YZdw),(Xq,Ca,YZdx),(Xq,Ca,YZdy),(Xq, Ca,YZdz),(Xq,Ca,YZea),(Xq,Ca,YZeb),(Xq,Ca,YZec),(Xq,Ca,YZed),(Xq,Ca,YZee),(Xq, Ca,YZef),(Xq,Ca,YZeg),(Xq,Ca,YZeh),(Xq,Ca,YZei),(Xq,Ca,YZej),(Xq,Ca,YZek), (Xq,Ca,YZel),(Xq,Ca,YZem),(Xq,Ca,YZen),(Xq,Ca,YZeo),(Xq,Ca,YZep),(Xq,Ca,YZeq), (Xq,Ca,YZer),(Xq,Ca,YZes),(Xq,Ca,YZet),(Xq,Ca,YZeu),(Xq,Ca,YZev),(Xq,Ca, YZew),(Xq,Ca,YZex),(Xq,Ca,YZey),(Xq,Ca,YZez),(Xq,Ca,YZfa),(Xq,Ca,YZfb),(Xq, Ca,YZfc),(Xq,Ca,YZfd),(Xq,Ca,YZfe),(Xq,Ca,YZff),(Xq,Ca,YZfg),(Xq,Ca,YZfh),(Xq, Ca,YZfi),(Xq,Ca,YZfj),(Xq,Ca,YZfk),(Xq,Ca,YZfl),(Xq,Ca,YZfm),(Xq,Ca,YZfn),(Xq, Ca,YZfo),(Xq,Ca,YZfp),(Xq,Ca,YZfq),(Xq,Ca,YZfr),(Xq,Ca,YZfs),(Xq,Ca,YZft),(Xq, Ca,YZfu),(Xq,Ca,YZfv),(Xq,Ca,YZfw),(Xq,Ca,YZfx),(Xq,Ca,YZfy),(Xq,Ca,YZfz),(Xq, Ca,YZga),(Xq,Ca,YZgb),(Xq,Ca,YZgc),(Xq,Ca,YZgd),(Xq,Ca,YZge),(Xq,Ca,YZgf), (Xq,Ca,YZgg),(Xq,Ca,YZgh),(Xq,Ca,YZgi),(Xq,Ca,YZgj),(Xq,Ca,YZgk),(Xq,Ca,YZgl), (Xq,Ca,YZgm),(Xq,Ca,YZgn),(Xq,Ca,YZgo),(Xq,Ca,YZgp),(Xq,Ca,YZgq),(Xq, Ca,YZgr),(Xq,Ca,YZgs),(Xq,Ca,YZgt),(Xq,Cb,YZa),(Xq,Cb,YZb),(Xq,Cb,YZc),(Xq, Cb,YZd),(Xq,Cb,YZe),(Xq,Cb,YZf),(Xq,Cb,YZg),(Xq,Cb,YZh),(Xq,Cb,YZi),(Xq,Cb, YZj),(Xq,Cb,YZk),(Xq,Cb,YZl),(Xq,Cb,YZm),(Xq,Cb,YZn),(Xq,Cb,YZo),(Xq,Cb,YZp), (Xq,Cb,YZq),(Xq,Cb,YZr),(Xq,Cb,YZs),(Xq,Cb,YZt),(Xq,Cb,YZu),(Xq,Cb,YZv),(Xq, Cb,YZw),(Xq,Cb,YZx),(Xq,Cb,YZy),(Xq,Cb,YZz),(Xq,Cb,YZaa),(Xq,Cb,YZab),(Xq, Cb,YZac),(Xq,Cb,YZad),(Xq,Cb,YZae),(Xq,Cb,YZaf),(Xq,Cb,YZag),(Xq,Cb,YZah), (Xq,Cb,YZai),(Xq,Cb,YZaj),(Xq,Cb,YZak),(Xq,Cb,YZal),(Xq,Cb,YZam),(Xq,Cb, YZan),(Xq,Cb,YZao),(Xq,Cb,YZap),(Xq,Cb,YZaq),(Xq,Cb,YZar),(Xq,Cb,YZas),(Xq, Cb,YZat),(Xq,Cb,YZau),(Xq,Cb,YZav),(Xq,Cb,YZaw),(Xq,Cb,YZax),(Xq,Cb,YZay), (Xq,Cb,YZaz),(Xq,Cb,YZba),(Xq,Cb,YZbb),(Xq,Cb,YZbc),(Xq,Cb,YZbd),(Xq,Cb,YZbe), (Xq,Cb,YZbf),(Xq,Cb,YZbg),(Xq,Cb,YZbh),(Xq,Cb,YZbi),(Xq,Cb,YZbj),(Xq,Cb, YZbk),(Xq,Cb,YZbl),(Xq,Cb,YZbm),(Xq,Cb,YZbn),(Xq,Cb,YZbo),(Xq,Cb,YZbp),(Xq, Cb,YZbq),(Xq,Cb,YZbr),(Xq,Cb,YZbs),(Xq,Cb,YZbt),(Xq,Cb,YZbu),(Xq,Cb,YZbv), (Xq,Cb,YZbw),(Xq,Cb,YZbx),(Xq,Cb,YZby),(Xq,Cb,YZbz),(Xq,Cb,YZca),(Xq,Cb, YZcb),(Xq,Cb,YZcc),(Xq,Cb,YZcd),(Xq,Cb,YZce),(Xq,Cb,YZcf),(Xq,Cb,YZcg),(Xq, Cb,YZch),(Xq,Cb,YZci),(Xq,Cb,YZcj),(Xq,Cb,YZck),(Xq,Cb,YZcl),(Xq,Cb,YZcm),(Xq, Cb,YZcn),(Xq,Cb,YZco),(Xq,Cb,YZcp),(Xq,Cb,YZcq),(Xq,Cb,YZcr),(Xq,Cb,YZcs), (Xq,Cb,YZct),(Xq,Cb,YZcu),(Xq,Cb,YZcv),(Xq,Cb,YZcw),(Xq,Cb,YZcx),(Xq,Cb, YZcy),(Xq,Cb,YZcz),(Xq,Cb,YZda),(Xq,Cb,YZdb),(Xq,Cb,YZdc),(Xq,Cb,YZdd),(Xq, Cb,YZde),(Xq,Cb,YZdf),(Xq,Cb,YZdg),(Xq,Cb,YZdh),(Xq,Cb,YZdi),(Xq,Cb,YZdj),(Xq, Cb,YZdk),(Xq,Cb,YZdl),(Xq,Cb,YZdm),(Xq,Cb,YZdn),(Xq,Cb,YZdo),(Xq,Cb,YZdp), (Xq,Cb,YZdq),(Xq,Cb,YZdr),(Xq,Cb,YZds),(Xq,Cb,YZdt),(Xq,Cb,YZdu),(Xq,Cb, YZdv),(Xq,Cb,YZdw),(Xq,Cb,YZdx),(Xq,Cb,YZdy),(Xq,Cb,YZdz),(Xq,Cb,YZea),(Xq, Cb,YZeb),(Xq,Cb,YZec),(Xq,Cb,YZed),(Xq,Cb,YZee),(Xq,Cb,YZef),(Xq,Cb,YZeg), (Xq,Cb,YZeh),(Xq,Cb,YZei),(Xq,Cb,YZej),(Xq,Cb,YZek),(Xq,Cb,YZel),(Xq,Cb,YZem), (Xq,Cb,YZen),(Xq,Cb,YZeo),(Xq,Cb,YZep),(Xq,Cb,YZeq),(Xq,Cb,YZer),(Xq,Cb, YZes),(Xq,Cb,YZet),(Xq,Cb,YZeu),(Xq,Cb,YZev),(Xq,Cb,YZew),(Xq,Cb,YZex),(Xq, Cb,YZey),(Xq,Cb,YZez),(Xq,Cb,YZfa),(Xq,Cb,YZfb),(Xq,Cb,YZfc),(Xq,Cb,YZfd),(Xq, Cb,YZfe),(Xq,Cb,YZff),(Xq,Cb,YZfg),(Xq,Cb,YZfh),(Xq,Cb,YZfi),(Xq,Cb,YZfj), (Xq,Cb,YZfk),(Xq,Cb,YZfl),(Xq,Cb,YZfm),(Xq,Cb,YZfn),(Xq,Cb,YZfo),(Xq,Cb,YZfp), (Xq,Cb,YZfq),(Xq,Cb,YZfr),(Xq,Cb,YZfs),(Xq,Cb,YZft),(Xq,Cb,YZfu),(Xq,Cb,YZfv), (Xq,Cb,YZfw),(Xq,Cb,YZfx),(Xq,Cb,YZfy),(Xq,Cb,YZfz),(Xq,Cb,YZga),(Xq,Cb, YZgb),(Xq,Cb,YZgc),(Xq,Cb,YZgd),(Xq,Cb,YZge),(Xq,Cb,YZgf),(Xq,Cb,YZgg),(Xq, Cb,YZgh),(Xq,Cb,YZgi),(Xq,Cb,YZgj),(Xq,Cb,YZgk),(Xq,Cb,YZgl),(Xq,Cb,YZgm), (Xq,Cb,YZgn),(Xq,Cb,YZgo),(Xq,Cb,YZgp),(Xq,Cb,YZgq),(Xq,Cb,YZgr),(Xq,Cb,YZgs), (Xq,Cb,YZgt),(Xq,Cc,YZa),(Xq,Cc,YZb),(Xq,Cc,YZc),(Xq,Cc,YZd),(Xq,Cc,YZe), (Xq,Cc,YZf),(Xq,Cc,YZg),(Xq,Cc,YZh),(Xq,Cc,YZi),(Xq,Cc,YZj),(Xq,Cc,YZk),(Xq, Cc,YZl),(Xq,Cc,YZm),(Xq,Cc,YZn),(Xq,Cc,YZo),(Xq,Cc,YZp),(Xq,Cc,YZq),(Xq,Cc, YZr),(Xq,Cc,YZs),(Xq,Cc,YZt),(Xq,Cc,YZu),(Xq,Cc,YZv),(Xq,Cc,YZw),(Xq,Cc,YZx), (Xq,Cc,YZy),(Xq,Cc,YZz),(Xq,Cc,YZaa),(Xq,Cc,YZab),(Xq,Cc,YZac),(Xq,Cc,YZad), (Xq,Cc,YZae),(Xq,Cc,YZaf),(Xq,Cc,YZag),(Xq,Cc,YZah),(Xq,Cc,YZai),(Xq,Cc, YZaj),(Xq,Cc,YZak),(Xq,Cc,YZal),(Xq,Cc,YZam),(Xq,Cc,YZan),(Xq,Cc,YZao),(Xq, Cc,YZap),(Xq,Cc,YZaq),(Xq,Cc,YZar),(Xq,Cc,YZas),(Xq,Cc,YZat),(Xq,Cc,YZau),(Xq, Cc,YZav),(Xq,CccYZaw),(XqCcc,YZax),(Xq,CccYZay),(Xq,Cc,YZaz),(Xq,Cc,YZba), (Xq,Cc,YZbb),(Xq,Cc,YZbc),(Xq,Cc,YZbd),(Xq,Cc,YZbe),(Xq,Cc,YZbf),(Xq,Cc,YZbg), (Xq,Cc,YZbh),(Xq,Cc,YZbi),(Xq,Cc,YZbj),(Xq,Cc,YZbk),(Xq,Cc,YZbl),(Xq,Cc, YZbm),(Xq,Cc,YZbn),(Xq,Cc,YZbo),(Xq,Cc,YZbp),(Xq,Cc,YZbq),(Xq,Cc,YZbr),(Xq, Cc,YZbs),(Xq,Cc,YZbt),(Xq,Cc,YZbu),(Xq,Cc,YZbv),(Xq,Cc,YZbw),(Xq,Cc,YZbx), (Xq,Cc,YZby),(Xq,Cc,YZbz),(Xq,Cc,YZca),(Xq,Cc,YZcb),(Xq,Cc,YZcc),(Xq,Cc,YZcd), (Xq,Cc,YZce),(Xq,Cc,YZcf),(Xq,Cc,YZcg),(Xq,Cc,YZch),(Xq,Cc,YZci),(Xq,Cc, YZcj),(Xq,Cc,YZck),(Xq,Cc,YZcl),(Xq,Cc,YZcm),(Xq,Cc,YZcn),(Xq,Cc,YZco),(Xq, Cc,YZcp),(Xq,Cc,YZcq),(Xq,Cc,YZcr),(Xq,Cc,YZcs),(Xq,Cc,YZct),(Xq,Cc,YZcu),(Xq, Cc,YZcv),(Xq,Cc,YZcw),(Xq,Cc,YZcx),(Xq,Cc,YZcy),(Xq,Cc,YZcz),(Xq,Cc,YZda), (Xq,Cc,YZdb),(Xq,Cc,YZdc),(Xq,Cc,YZdd),(Xq,Cc,YZde),(Xq,Cc,YZdf),(Xq,Cc,YZdg), (Xq,Cc,YZdh),(Xq,Cc,YZdi),(Xq,Cc,YZdj),(Xq,Cc,YZdk),(Xq,Cc,YZdl),(Xq,Cc,YZdm), (Xq,Cc,YZdn),(Xq,Cc,YZdo),(Xq,Cc,YZdp),(Xq,Cc,YZdq),(Xq,Cc,YZdr),(Xq, Cc,YZds),(Xq,Cc,YZdt),(Xq,Cc,YZdu),(Xq,Cc,YZdv),(Xq,Cc,YZdw),(Xq,Cc,YZdx), (Xq,Cc,YZdy),(Xq,Cc,YZdz),(Xq,Cc,YZea),(Xq,Cc,YZeb),(Xq,Cc,YZec),(Xq,Cc,YZed), (Xq,Cc,YZee),(Xq,Cc,YZef),(Xq,Cc,YZeg),(Xq,Cc,YZeh),(Xq,Cc,YZei),(Xq,Cc, YZej),(Xq,Cc,YZek),(Xq,Cc,YZel),(Xq,Cc,YZem),(Xq,Cc,YZen),(Xq,Cc,YZeo),(Xq, Cc,YZep),(Xq,Cc,YZeq),(Xq,Cc,YZer),(Xq,Cc,YZes),(Xq,Cc,YZet),(Xq,Cc,YZeu),(Xq, Cc,YZev),(Xq,Cc,YZew),(Xq,Cc,YZex),(Xq,Cc,YZey),(Xq,Cc,YZez),(Xq,Cc,YZfa), (Xq,Cc,YZfb),(Xq,Cc,YZfc),(Xq,Cc,YZfd),(Xq,Cc,YZfe),(Xq,Cc,YZff),(Xq,Cc,YZfg), (Xq,Cc,YZfh),(Xq,Cc,YZfi),(Xq,Cc,YZfj),(Xq,Cc,YZfk),(Xq,Cc,YZfl),(Xq,Cc,YZfm), (Xq,Cc,YZfn),(Xq,Cc,YZfo),(Xq,Cc,YZfp),(Xq,Cc,YZfq),(Xq,Cc,YZfr),(Xq,Cc,YZfs), (Xq,Cc,YZft),(Xq,Cc,YZfu),(Xq,Cc,YZfv),(Xq,Cc,YZfw),(Xq,Cc,YZfx),(Xq,Cc, YZfy),(Xq,Cc,YZfz),(Xq,Cc,YZga),(Xq,Cc,YZgb),(Xq,Cc,YZgc),(Xq,Cc,YZgd),(Xq, Cc,YZge),(Xq,Cc,YZgf),(Xq,Cc,YZgg),(Xq,Cc,YZgh),(Xq,Cc,YZgi),(Xq,Cc,YZgj),(Xq, Cc,YZgk),(Xq,Cc,YZgl),(Xq,Cc,YZgm),(Xq,Cc,YZgn),(Xq,Cc,YZgo),(Xq,Cc,YZgp), (Xq,Cc,YZgq),(Xq,Cc,YZgr),(Xq,Cc,YZgs),(Xq,Cc,YZgt),(Xq,Cd,YZa),(Xq,Cd,YZb), (Xq,Cd,YZc),(Xq,Cd,YZd),(Xq,Cd,YZe),(Xq,Cd,YZf),(Xq,Cd,YZg),(Xq,Cd,YZh), (Xq,Cd,YZi),(Xq,Cd,YZj),(Xq,Cd,YZk),(Xq,Cd,YZl),(Xq,Cd,YZm),(Xq,Cd,YZn),(Xq, Cd,YZo),(Xq,Cd,YZp),(Xq,Cd,YZq),(Xq,Cd,YZr),(Xq,Cd,YZs),(Xq,Cd,YZt),(Xq,Cd, YZu),(Xq,Cd,YZv),(Xq,Cd,YZw),(Xq,Cd,YZx),(Xq,Cd,YZy),(Xq,Cd,YZz),(Xq,Cd, YZaa),(Xq,Cd,YZab),(Xq,Cd,YZac),(Xq,Cd,YZad),(Xq,Cd,YZae),(Xq,Cd,YZaf),(Xq, Cd,YZag),(Xq,Cd,YZah),(Xq,Cd,YZai),(Xq,Cd,YZaj),(Xq,Cd,YZak),(Xq,Cd,YZal),(Xq, Cd,YZam),(Xq,Cd,YZan),(Xq,Cd,YZao),(Xq,Cd,YZap),(Xq,Cd,YZaq),(Xq,Cd,YZar), (Xq,Cd,YZas),(Xq,Cd,YZat),(Xq,Cd,YZau),(Xq,Cd,YZav),(Xq,Cd,YZaw),(Xq,Cd, YZax),(Xq,Cd,YZay),(Xq,Cd,YZaz),(Xq,Cd,YZba),(Xq,Cd,YZbb),(Xq,Cd,YZbc),(Xq, Cd,YZbd),(Xq,Cd,YZbe),(Xq,Cd,YZbf),(Xq,Cd,YZbg),(Xq,Cd,YZbh),(Xq,Cd,YZbi), (Xq,Cd,YZbj),(Xq,Cd,YZbk),(Xq,Cd,YZbl),(Xq,Cd,YZbm),(Xq,Cd,YZbn),(Xq,Cd,YZbo), (Xq,Cd,YZbp),(Xq,Cd,YZbq),(Xq,Cd,YZbr),(Xq,Cd,YZbs),(Xq,Cd,YZbt),(Xq,Cd, YZbu),(Xq,Cd,YZbv),(Xq,Cd,YZbw),(Xq,Cd,YZbx),(Xq,Cd,YZby),(Xq,Cd,YZbz),(Xq, Cd,YZca),(Xq,Cd,YZcb),(Xq,Cd,YZcc),(Xq,Cd,YZcd),(Xq,Cd,YZce),(Xq,Cd,YZcf), (Xq,Cd,YZcg),(Xq,Cd,YZch),(Xq,Cd,YZci),(Xq,Cd,YZcj),(Xq,Cd,YZck),(Xq,Cd,YZcl), (Xq,Cd,YZcm),(Xq,Cd,YZcn),(Xq,Cd,YZco),(Xq,Cd,YZcp),(Xq,Cd,YZcq),(Xq, Cd,YZcr),(Xq,Cd,YZcs),(Xq,Cd,YZct),(Xq,Cd,YZcu),(Xq,Cd,YZcv),(Xq,Cd,YZcw),(Xq, Cd,YZcx),(Xq,Cd,YZcy),(Xq,Cd,YZcz),(Xq,Cd,YZda),(Xq,Cd,YZdb),(Xq,Cd,YZdc), (Xq,Cd,YZdd),(Xq,Cd,YZde),(Xq,Cd,YZdf),(Xq,Cd,YZdg),(Xq,Cd,YZdh),(Xq,Cd, YZdi),(Xq,Cd,YZdj),(Xq,Cd,YZdk),(Xq,Cd,YZdl),(Xq,Cd,YZdm),(Xq,Cd,YZdn),(Xq, Cd,YZdo),(Xq,Cd,YZdp),(Xq,Cd,YZdq),(Xq,Cd,YZdr),(Xq,Cd,YZds),(Xq,Cd,YZdt), (Xq,Cd,YZdu),(Xq,Cd,YZdv),(Xq,Cd,YZdw),(Xq,Cd,YZdx),(Xq,Cd,YZdy),(Xq,Cd, YZdz),(Xq,Cd,YZea),(Xq,Cd,YZeb),(Xq,Cd,YZec),(Xq,Cd,YZed),(Xq,Cd,YZee),(Xq, Cd,YZef),(Xq,Cd,YZeg),(Xq,Cd,YZeh),(Xq,Cd,YZei),(Xq,Cd,YZej),(Xq,Cd,YZek),(Xq, Cd,YZel),(Xq,Cd,YZem),(Xq,Cd,YZen),(Xq,Cd,YZeo),(Xq,Cd,YZep),(Xq,Cd,YZeq), (Xq,Cd,YZer),(Xq,Cd,YZes),(Xq,Cd,YZet),(Xq,Cd,YZeu),(Xq,Cd,YZev),(Xq,Cd, YZew),(Xq,Cd,YZex),(Xq,Cd,YZey),(Xq,Cd,YZez),(Xq,Cd,YZfa),(Xq,Cd,YZfb),(Xq, Cd,YZfc),(Xq,Cd,YZfd),(Xq,Cd,YZfe),(Xq,Cd,YZff),(Xq,Cd,YZfg),(Xq,Cd,YZfh),(Xq, Cd,YZfi),(Xq,Cd,YZfj),(Xq,Cd,YZfk),(Xq,Cd,YZfl),(Xq,Cd,YZfm),(Xq,Cd,YZfn),(Xq, Cd,YZfo),(Xq,Cd,YZfp)),(Xq,Cd,YZfq),(Xq,Cd,YZfr),(Xq,Cd,YZfs),(Xq,Cd,YZft), (Xq,Cd,YZfu),(Xq,Cd,YZfv),(Xq,Cd,YZfw),(Xq,Cd,YZfx),(Xq,Cd,YZfy),(Xq,Cd,YZfz), (Xq,Cd,YZga),(Xq,Cd,YZgb),(Xq,Cd,YZgc),(Xq,Cd,YZgd),(Xq,Cd,YZge),(Xq,Cd, YZgf),(Xq,Cd,YZgg),(Xq,Cd,YZgh),(Xq,Cd,YZgi),(Xq,Cd,YZgj),(Xq,Cd,YZgk),(Xq, Cd,YZgl),(Xq,Cd,YZgm),(Xq,Cd,YZgn),(Xq,Cd,YZgo),(Xq,Cd,YZgp),(Xq,Cd,YZgq), (Xq,Cd,YZgr),(Xq,Cd,YZgs),(Xq,Cd,YZgt),(Xq,Ce,YZa),(Xq,Ce,YZb),(Xq,Ce,YZc), (Xq,Ce,YZd),(Xq,Ce,YZe),(Xq,Ce,YZf),(Xq,Ce,YZg),(Xq,Ce,YZh),(Xq,Ce,YZi),(Xq, Ce,YZj),(Xq,Ce,YZk),(Xq,Ce,YZl),(Xq,Ce,YZm),(Xq,Ce,YZn),(Xq,Ce,YZo),(Xq,Ce, YZp),(Xq,Ce,YZq),(Xq,Ce,YZr),(Xq,Ce,YZs),(Xq,Ce,YZt),(Xq,Ce,YZu),(Xq,Ce,YZv), (Xq,Ce,YZw),(Xq,Ce,YZx),(Xq,Ce,YZy),(Xq,Ce,YZz),(Xq,Ce,YZaa),(Xq,Ce,YZab), (Xq,Ce,YZac),(Xq,Ce,YZad),(Xq,Ce,YZae),(Xq,Ce,YZaf),(Xq,Ce,YZag),(Xq,Ce,YZah), (Xq,Ce,YZai),(Xq,Ce,YZaj),(Xq,Ce,YZak),(Xq,Ce,YZal),(Xq,Ce,YZam),(Xq,Ce, YZan),(Xq,Ce,YZao),(Xq,Ce,YZap),(Xq,Ce,YZaq),(Xq,Ce,YZar),(Xq,Ce,YZas),(Xq, Ce,YZat),(Xq,Ce,YZau),(Xq,Ce,YZav),(Xq,Ce,YZaw),(Xq,Ce,YZax),(Xq,Ce,YZay),(Xq, Ce,YZaz),(Xq,Ce,YZba),(Xq,Ce,YZbb),(Xq,Ce,YZbc),(Xq,Ce,YZbd),(Xq,Ce,YZbe), (Xq,Ce,YZbf),(Xq,Ce,YZbg),(Xq,Ce,YZbh),(Xq,Ce,YZbi),(Xq,Ce,YZbj),(Xq,Ce,YZbk), (Xq,Ce,YZbl),(Xq,Ce,YZbm),(Xq,Ce,YZbn),(Xq,Ce,YZbo),(Xq,Ce,YZbp),(Xq, Ce,YZbq),(Xq,Ce,YZbr),(Xq,Ce,YZbs),(Xq,Ce,YZbt),(Xq,Ce,YZbu),(Xq,Ce,YZbv),(Xq, Ce,YZbw),(Xq,Ce,YZbx),(Xq,Ce,YZby),(Xq,Ce,YZbz),(Xq,Ce,YZca),(Xq,Ce,YZcb), (Xq,Ce,YZcc),(Xq,Ce,YZcd),(Xq,Ce,YZce),(Xq,Ce,YZcf),(Xq,Ce,YZcg),(Xq,Ce, YZch),(Xq,Ce,YZci),(Xq,Ce,YZcj),(Xq,Ce,YZck),(Xq,Ce,YZcl),(Xq,Ce,YZcm),(Xq, Ce,YZcn),(Xq,Ce,YZco),(Xq,Ce,YZcp),(Xq,Ce,YZcq),(Xq,Ce,YZcr),(Xq,Ce,YZcs),(Xq, Ce,YZct),(Xq,Ce,YZcu),(Xq,Ce,YZcv),(Xq,Ce,YZcw),(Xq,Ce,YZcx),(Xq,Ce,YZcy), (Xq,Ce,YZcz),(Xq,Ce,YZda),(Xq,Ce,YZdb),(Xq,Ce,YZdc),(Xq,Ce,YZdd),(Xq,Ce, YZde),(Xq,Ce,YZdf),(Xq,Ce,YZdg),(Xq,Ce,YZdh),(Xq,Ce,YZdi),(Xq,Ce,YZdj),(Xq, Ce,YZdk),(Xq,Ce,YZdl),(Xq,Ce,YZdm),(Xq,Ce,YZdn),(Xq,Ce,YZdo),(Xq,Ce,YZdp), (Xq,Ce,YZdq),(Xq,Ce,YZdr),(Xq,Ce,YZds),(Xq,Ce,YZdt),(Xq,Ce,YZdu),(Xq,Ce,YZdv), (Xq,Ce,YZdw),(Xq,Ce,YZdx),(Xq,Ce,YZdy),(Xq,Ce,YZdz),(Xq,Ce,YZea),(Xq,Ce, YZeb),(Xq,Ce,YZec),(Xq,Ce,YZed),(Xq,Ce,YZee),(Xq,Ce,YZef),(Xq,Ce,YZeg),(Xq, Ce,YZeh),(Xq,Ce,YZei),(Xq,Ce,YZej),(Xq,Ce,YZek),(Xq,Ce,YZel),(Xq,Ce,YZem),(Xq, Ce,YZen),(Xq,Ce,YZeo),(Xq,Ce,YZep),(Xq,Ce,YZeq),(Xq,Ce,YZer),(Xq,Ce,YZes), (Xq,Ce,YZet),(Xq,Ce,YZeu),(Xq,Ce,YZev),(Xq,Ce,YZew),(Xq,Ce,YZex),(Xq,Ce,YZey), (Xq,Ce,YZez),(Xq,Ce,YZfa),(Xq,Ce,YZfb),(Xq,Ce,YZfc),(Xq,Ce,YZfd),(Xq,Ce, YZfe),(Xq,Ce,YZff),(Xq,Ce,YZfg),(Xq,Ce,YZfh),(Xq,Ce,YZfi),(Xq,Ce,YZfj),(Xq,Ce, YZfk),(Xq,Ce,YZfl),(Xq,Ce,YZfm),(Xq,Ce,YZfn),(Xq,Ce,YZfo),(Xq,Ce,YZfp),(Xq,Ce, YZfq),(Xq,Ce,YZfr),(Xq,Ce,YZfs),(Xq,Ce,YZft),(Xq,Ce,YZfu),(Xq,Ce,YZfv),(Xq,Ce, YZfw),(Xq,Ce,YZfx),(Xq,Ce,YZfy),(Xq,Ce,YZfz),(Xq,Ce,YZga),(Xq,Ce,YZgb),(Xq, Ce,YZgc),(Xq,Ce,YZgd),(Xq,Ce,YZge),(Xq,Ce,YZgf),(Xq,Ce,YZgg),(Xq,Ce,YZgh), (Xq,Ce,YZgi),(Xq,Ce,YZgj),(Xq,Ce,YZgk),(Xq,Ce,YZgl),(Xq,Ce,YZgm),(Xq,Ce,YZgn), (Xq,Ce,YZgo),(Xq,Ce,YZgp),(Xq,Ce,YZgq),(Xq,Ce,YZgr),(Xq,Ce,YZgs),(Xq,Ce, YZgt),(Xq,Cf,YZa),(Xq,Cf,YZb),(Xq,Cf,YZc),(Xq,Cf,YZd),(Xq,Cf,YZe),(Xq,Cf,YZf), (Xq,Cf,YZg),(Xq,Cf,YZh),(Xq,Cf,YZi),(Xq,Cf,YZj),(Xq,Cf,YZk),(Xq,Cf,YZl),(Xq,Cf, YZm),(Xq,Cf,YZn),(Xq,Cf,YZo),(Xq,Cf,YZp),(Xq,Cf,YZq),(Xq,Cf,YZr),(Xq,Cf,YZs), (Xq,Cf,YZt),(Xq,Cf,YZu),(Xq,Cf,YZv),(Xq,Cf,YZw),(Xq,Cf,YZx),(Xq,Cf,YZy),(Xq, Cf,YZz),(Xq,Cf,YZaa),(Xq,Cf,YZab),(Xq,Cf,YZac),(Xq,Cf,YZad),(Xq,Cf,YZae),(Xq, Cf,YZaf),(Xq,Cf,YZag),(Xq,Cf,YZah),(Xq,Cf,YZai),(Xq,Cf,YZaj),(Xq,Cf,YZak),(Xq, Cf,YZal),(Xq,Cf,YZam),(Xq,Cf,YZan),(Xq,Cf,YZao),(Xq,Cf,YZap),(Xq,Cf,YZaq),(Xq, Cf,YZar),(Xq,Cf,YZas),(Xq,Cf,YZat),(Xq,Cf,YZau),(Xq,Cf,YZav),(Xq,Cf,YZaw), (Xq,Cf,YZax),(Xq,Cf,YZay),(Xq,Cf,YZaz),(Xq,Cf,YZba),(Xq,Cf,YZbb),(Xq,Cf,YZbc), (Xq,Cf,YZbd),(Xq,Cf,YZbe),(Xq,Cf,YZbf),(Xq,Cf,YZbg),(Xq,Cf,YZbh),(Xq,Cf,YZbi), (Xq,Cf,YZbj),(Xq,Cf,YZbk),(Xq,Cf,YZbl),(Xq,Cf,YZbm),(Xq,Cf,YZbn),(Xq,Cf,YZbo), (Xq,Cf,YZbp),(Xq,Cf,YZbq),(Xq,Cf,YZbr),(Xq,Cf,YZbs),(Xq,Cf,YZbt),(Xq,Cf, YZbu),(Xq,Cf,YZbv),(Xq,Cf,YZbw),(Xq,Cf,YZbx),(Xq,Cf,YZby),(Xq,Cf,YZbz),(Xq, Cf,YZca),(Xq,Cf,YZcb),(Xq,Cf,YZcc),(Xq,Cf,YZcd),(Xq,Cf,YZce),(Xq,Cf,YZcf),(Xq, Cf,YZcg),(Xq,Cf,YZch),(Xq,Cf,YZci),(Xq,Cf,YZcj),(Xq,Cf,YZck),(Xq,Cf,YZcl),(Xq, Cf,YZcm),(Xq,Cf,YZcn),(Xq,Cf,YZco),(Xq,Cf,YZcp),(Xq,Cf,YZcq),(Xq,Cf,YZcr),(Xq, Cf,YZcs),(Xq,Cf,YZct),(Xq,Cf,YZcu),(Xq,Cf,YZcv),(Xq,Cf,YZcw),(Xq,Cf,YZcx), (Xq,Cf,YZcy),(Xq,Cf,YZcz),(Xq,Cf,YZda),(Xq,Cf,YZdb),(Xq,Cf,YZdc),(Xq,Cf,YZdd), (Xq,Cf,YZde),(Xq,Cf,YZdf),(Xq,Cf,YZdg),(Xq,Cf,YZdh),(Xq,Cf,YZdi),(Xq,Cf,YZdj), (Xq,Cf,YZdk),(Xq,Cf,YZdl),(Xq,Cf,YZdm),(Xq,Cf,YZdn),(Xq,Cf,YZdo),(Xq,Cf, YZdp),(Xq,Cf,YZdq),(Xq,Cf,YZdr),(Xq,Cf,YZds),(Xq,Cf,YZdt),(Xq,Cf,YZdu),(Xq,Cf, YZdv),(Xq,Cf,YZdw),(Xq,Cf,YZdx),(Xq,Cf,YZdy),(Xq,Cf,YZdz),(Xq,Cf,YZea),(Xq, Cf,YZeb),(Xq,Cf,YZec),(Xq,Cf,YZed),(Xq,Cf,YZee),(Xq,Cf,YZef),(Xq,Cf,YZeg),(Xq, Cf,YZeh),(Xq,Cf,YZei),(Xq,Cf,YZej),(Xq,Cf,YZek),(Xq,Cf,YZel),(Xq,Cf,YZem),(Xq, Cf,YZen),(Xq,Cf,YZeo),(Xq,Cf,YZep),(Xq,Cf,YZeq),(Xq,Cf,YZer),(Xq,Cf,YZes),(Xq, Cf,YZet),(Xq,Cf,YZeu),(Xq,Cf,YZev),(Xq,Cf,YZew),(Xq,Cf,YZex),(Xq,Cf,YZey), (Xq,Cf,YZez),(Xq,Cf,YZfa),(Xq,Cf,YZfb),(Xq,Cf,YZfc),(Xq,Cf,YZfd),(Xq,Cf,YZfe), (Xq,Cf,YZff),(Xq,Cf,YZfg),(Xq,Cf,YZfh),(Xq,Cf,YZfi),(Xq,Cf,YZfj),(Xq,Cf,YZfk),(Xq, Cf,YZfl),(Xq,Cf,YZfm),(Xq,Cf,YZfn),(Xq,Cf,YZfo),(Xq,Cf,YZfcp),(Xq,Cf,YZfq),(Xq, Cf,YZfr),(Xq,Cf,YZfs),(Xq,Cf,YZft),(Xq,Cf,YZfu),(Xq,Cf,YZfv),(Xq,Cf,YZfw),(Xq, Cf,YZfx),(Xq,Cf,YZfy),(Xq,Cf,YZfz),(Xq,Cf,YZga),(Xq,Cf,YZgb),(Xq,Cf,YZgc), (Xq,Cf,YZgd),(Xq,Cf,YZge),(Xq,Cf,YZgf),(Xq,Cf,YZgg),(Xq,Cf,YZgh),(Xq,Cf,YZgi), (Xq,Cf,YZgj),(Xq,Cf,YZgk),(Xq,Cf,YZgl),(Xq,Cf,YZgm),(Xq,Cf,YZgn),(Xq,Cf,YZgo), (Xq,Cf,YZgp),(Xq,Cf,YZgq),(Xq,Cf,YZgr),(Xq,Cf,YZgs),(Xq,Cf,YZgt), (Xr,Ca,YZa),(Xr,Ca,YZb),(Xr,Ca,YZc),(Xr,Ca,YZd),(Xr,Ca,YZe),(Xr,Ca,YZf),(Xr,Ca, YZg),(Xr,Ca,YZh),(Xr,Ca,YZi),(Xr,Ca,YZj),(Xr,Ca,YZk),(Xr,Ca,YZl),(Xr,Ca,YZm), (Xr,Ca,YZn),(Xr,Ca,YZo),(Xr,Ca,YZp),(Xr,Ca,YZq),(Xr,Ca,YZr),(Xr,Ca,YZs),(Xr,Ca, YZt),(Xr,Ca,YZu),(Xr,Ca,YZv),(Xr,Ca,YZw),(Xr,Ca,YZx),(Xr,Ca,YZy),(Xr,Ca,YZz), (Xr,Ca,YZaa),(Xr,Ca,YZab),(Xr,Ca,YZac),(Xr,Ca,YZad),(Xr,Ca,YZae),(Xr,Ca,YZaf), (Xr,Ca,YZag),(Xr,Ca,YZah),(Xr,Ca,YZai),(Xr,Ca,YZaj),(Xr,Ca,YZak),(Xr,Ca,YZal), (Xr,Ca,YZam),(Xr,Ca,YZan),(Xr,Ca,YZao),(Xr,Ca,YZap),(Xr,Ca,YZaq),(Xr,Ca,YZar), (Xr,Ca,YZas),(Xr,Ca,YZat),(Xr,Ca,YZau),(Xr,Ca,YZav),(Xr,Ca,YZaw),(Xr,Ca,YZax), (Xr,Ca,YZay),(Xr,Ca,YZaz),(Xr,Ca,YZba),(Xr,Ca,YZbb),(Xr,Ca,YZbc),(Xr,Ca,YZbd), (Xr,Ca,Yibe),(Xr,Ca,YZbf),(Xr,Ca,YZbg),(Xr,Ca,YZbh),(Xr,Ca,YZbi),(Xr,Ca,YZbj), (Xr,Ca,YZbk),(Xr,Ca,YZbl),(Xr,Ca,YZbm),(Xr,Ca,YZbn),(Xr,Ca,YZbo),(Xr,Ca, YZbp),(Xr,Ca,YZbq),(Xr,Ca,YZbr),(Xr,Ca,YZbs),(Xr,Ca,YZbt),(Xr,Ca,YZbu),(Xr,Ca, YZbv),(Xr,Ca,YZbw),(Xr,Ca,YZbx),(Xr,Ca,YZby),(Xr,Ca,YZbz),(Xr,Ca,YZca),(Xr,Ca, YZcb),(Xr,Ca,YZcc),(Xr,Ca,YZcd),(Xr,Ca,YZce),(Xr,Ca,YZcf),(Xr,Ca,YZcg),(Xr,Ca, YZch),(Xr,Ca,YZci),(Xr,Ca,YZcj),(Xr,Ca,YZck),(Xr,Ca,YZcl),(Xr,Ca,YZcm),(Xr,Ca, YZcn),(Xr,Ca,YZco),(Xr,Ca,YZcp),(Xr,Ca,YZcq),(Xr,Ca,YZcr),(Xr,Ca,YZcs),(Xr,Ca, YZct),(Xr,Ca,YZcu),(Xr,Ca,YZcv),(Xr,Ca,YZcw),(Xr,Ca,YZcx),(Xr,Ca,YZcy),(Xr, Ca,YZcz),(Xr,Ca,YZda),(Xr,Ca,YZdb),(Xr,Ca,YZdc),(Xr,Ca,YZdd),(Xr,Ca,YZde),(Xr, Ca,YZdf),(Xr,Ca,YZdg),(Xr,Ca,YZdh),(Xr,Ca,YZdi),(Xr,Ca,YZdj),(Xr,Ca,YZdk),(Xr, Ca,YZdl),(Xr,Ca,YZdm),(Xr,Ca,YZdn),(Xr,Ca,YZdo),(Xr,Ca,YZdp),(Xr,Ca,YZdq),(Xr, Ca,YZdr),(Xr,Ca,YZds),(Xr,Ca,YZdt),(Xr,Ca,YZdu),(Xr,Ca,YZdv),(Xr,Ca,YZdw), (Xr,Ca,YZdx),(Xr,Ca,YZdy),(Xr,Ca,YZdz),(Xr,Ca,YZea),(Xr,Ca,YZeb),(Xr,Ca,YZec), (Xr,Ca,YZed),(Xr,Ca,YZee),(Xr,Ca,YZef),(Xr,Ca,YZeg),(Xr,Ca,YZeh),(Xr,Ca,YZei), (Xr,Ca,YZej),(Xr,Ca,YZek),(Xr,Ca,YZel),(Xr,Ca,YZem),(Xr,Ca,YZen),(Xr,Ca,YZeo), (Xr,Ca,YZep),(Xr,Ca,YZeq),(Xr,Ca,YZer),(Xr,Ca,YZes),(Xr,Ca,YZet),(Xr,Ca,YZeu),(Xr, Ca,YZev),(Xr,Ca,YZew),(Xr,Ca,YZex),(Xr,Ca,YZey),(Xr,Ca,YZez),(Xr,Ca,YZfa), (Xr,Ca,YZfb),(Xr,Ca,YZfc),(Xr,Ca,YZfd),(Xr,Ca,YZfe),(Xr,Ca,YZff),(Xr,Ca,YZfg),(Xr, Ca,YZfh),(Xr,Ca,YZfi),(Xr,Ca,YZfj),(Xr,Ca,YZfk),(Xr,Ca,YZfl),(Xr,Ca,YZfm),(Xr, Ca,YZfn),(Xr,Ca,YZfo),(Xr,Ca,YZfp),(Xr,Ca,YZfq),(Xr,Ca,YZfr),(Xr,Ca,YZfs),(Xr, Ca,YZft),(Xr,Ca,YZfu),(Xr,Ca,YZfv),(Xr,Ca,YZfw),(Xr,Ca,YZfx),(Xr,Ca,YZfy),(Xr, Ca,YZfz),(Xr,Ca,YZga),(Xr,Ca,YZgb),(Xr,Ca,YZgc),(Xr,Ca,YZgd),(Xr,Ca,YZge),(Xr, Ca,YZgf),(Xr,Ca,YZgg),(Xr,Ca,YZgh),(Xr,Ca,YZgi),(Xr,Ca,YZgj),(Xr,Ca,YZgk),(Xr, Ca,YZgl),(Xr,Ca,YZgm),(Xr,Ca,YZgn),(Xr,Ca,YZgo),(Xr,Ca,YZgp),(Xr,Ca,YZgq),(Xr, Ca,YZgr),(Xr,Ca,YZgs),(Xr,Ca,YZgt),(Xr,Cb,YZa),(Xr,Cb,YZb),(Xr,Cb,YZc),(Xr, Cb,YZd),(Xr,Cb,YZe),(Xr,Cb,YZf),(Xr,Cb,YZg),(Xr,Cb,YZh),(Xr,Cb,YZi),(Xr,Cb,YZj), (Xr,Cb,YZk),(Xr,Cb,YZl),(Xr,Cb,YZm),(Xr,Cb,YZn),(Xr,Cb,YZo),(Xr,Cb,YZp),(Xr, Cb,YZq),(Xr,Cb,YZr),(Xr,Cb,YZs),(Xr,Cb,YZt),(Xr,Cb,YZu),(Xr,Cb,YZv),(Xr,Cb, YZw),(Xr,Cb,YZx),(Xr,Cb,YZy),(Xr,Cb,YZz),(Xr,Cb,YZaa),(Xr,Cb,YZab),(Xr,Cb,YZac), (Xr,Cb,YZad),(Xr,Cb,YZae),(Xr,Cb,YZaf),(Xr,Cb,YZag),(Xr,Cb,YZah),(Xr,Cb, YZai),(Xr,Cb,YZaj),(Xr,Cb,YZak),(Xr,Cb,YZal),(Xr,Cb,YZam),(Xr,Cb,YZan),(Xr,Cb, YZao),(Xr,Cb,YZap),(Xr,Cb,YZaq),(Xr,Cb,YZar),(Xr,Cb,YZas),(Xr,Cb,YZat),(Xr,Cb, YZau),(Xr,Cb,YZav),(Xr,Cb,YZaw),(Xr,Cb,YZax),(Xr,Cb,YZay),(Xr,Cb,YZaz),(Xr, Cb,YZba),(Xr,Cb,YZbb),(Xr,Cb,YZbc),(Xr,Cb,YZbd),(Xr,Cb,YZbe),(Xr,Cb,YZbf),(Xr, Cb,YZbg),(Xr,Cb,YZbh),(Xr,Cb,YZbi),(Xr,Cb,YZbj),(Xr,Cb,YZbk),(Xr,Cb,YZbl),(Xr, Cb,YZbm),(Xr,Cb,YZbn),(Xr,Cb,YZbo),(Xr,Cb,YZbp),(Xr,Cb,YZbq),(Xr,Cb,YZbr), (Xr,Cb,YZbs),(Xr,Cb,YZbt),(Xr,Cb,YZbu),(Xr,Cb,YZbv),(Xr,Cb,YZbw),(Xr,Cb,YZbx), (Xr,Cb,YZby),(Xr,Cb,YZbz),(Xr,Cb,YZca),(Xr,Cb,YZcb),(Xr,Cb,YZcc),(Xr,Cb, YZcd),(Xr,Cb,YZce),(Xr,Cb,YZcf),(Xr,Cb,YZcg),(Xr,Cb,YZch),(Xr,Cb,YZci),(Xr,Cb, YZcj),(Xr,Cb,YZck),(Xr,Cb,YZcl),(Xr,Cb,YZcm),(Xr,Cb,YZcn),(Xr,Cb,YZco),(Xr,Cb, YZcp),(Xr,Cb,YZcq),(Xr,Cb,YZcr),(Xr,Cb,YZcs),(Xr,Cb,YZct),(Xr,Cb,YZcu),(Xr,Cb, YZcv),(Xr,Cb,YZcw),(Xr,Cb,YZcx),(Xr,Cb,YZcy),(Xr,Cb,YZcz),(Xr,Cb,YZda),(Xr, Cb,YZdb),(Xr,Cb,YZdc),(Xr,Cb,YZdd),(Xr,Cb,YZde),(Xr,Cb,YZdf),(Xr,Cb,YZdg),(Xr, Cb,YZdh),(Xr,Cb,YZdi),(Xr,Cb,YZdj),(Xr,Cb,YZdk),(Xr,Cb,YZdl),(Xr,Cb,YZdm),(Xr, Cb,YZdn),(Xr,Cb,YZdo),(Xr,Cb,YZdp),(Xr,Cb,YZdq),(Xr,Cb,YZdr),(Xr,Cb,YZds), (Xr,Cb,YZdt),(Xr,Cb,YZdu),(Xr,Cb,YZdv),(Xr,Cb,YZdw),(Xr,Cb,YZdx),(Xr,Cb,YZdy), (Xr,Cb,YZdz),(Xr,Cb,YZea),(Xr,Cb,YZeb),(Xr,Cb,YZec),(Xr,Cb,YZed),(Xr,Cb,YZee), (Xr,Cb,YZef),(Xr,Cb,YZeg),(Xr,Cb,YZeh),(Xr,Cb,YZei),(Xr,Cb,YZej),(Xr,Cb,YZek), (Xr,Cb,YZel),(Xr,Cb,YZem),(Xr,Cb,YZen),(Xr,Cb,YZeo),(Xr,Cb,YZep),(Xr,Cb,YZeq), (Xr,Cb,YZer),(Xr,Cb,YZes),(Xr,Cb,YZet),(Xr,Cb,YZeu),(Xr,Cb,YZev),(Xr,Cb,YZew), (Xr,Cb,YZex),(Xr,Cb,YZey),(Xr,Cb,YZez),(Xr,Cb,YZfa),(Xr,Cb,YZfb),(Xr,Cb, YZfc),(Xr,Cb,YZfd),(Xr,Cb,YZfe),(Xr,Cb,YZff),(Xr,Cb,YZfg),(Xr,Cb,YZfh),(Xr,Cb, YZfi),(Xr,Cb,YZfj),(Xr,Cb,YZfk),(Xr,Cb,YZfl),(Xr,Cb,YZfm),(Xr,Cb,YZfn),(Xr,Cb, YZfo),(Xr,Cb,YZfp),(Xr,Cb,YZfq),(Xr,Cb,YZfr),(Xr,Cb,YZfs),(Xr,Cb,YZft),(Xr,Cb, YZfu),(Xr,Cb,YZfv),(Xr,Cb,YZfw),(Xr,Cb,YZfx),(Xr,Cb,YZfy),(Xr,Cb,YZfz),(Xr,Cb, YZga),(Xr,Cb,YZgb),(Xr,Cb,YZgc),(Xr,Cb,YZgd),(Xr,Cb,YZge),(Xr,Cb,YZgf),(Xr,Cb, YZgg),(Xr,Cb,YZgh),(Xr,Cb,YZgi),(Xr,Cb,YZgj),(Xr,Cb,YZgk),(Xr,Cb,YZgl),(Xr, Cb,YZgm),(Xr,Cb,YZgn),(Xr,Cb,YZgo),(Xr,Cb,YZgp),(Xr,Cb,YZgq),(Xr,Cb,YZgr),(Xr, Cb,YZgs),(Xr,Cb,YZgt),(Xr,Cc,YZa),(Xr,Cc,YZb),(Xr,Cc,YZc),(Xr,Cc,YZd),(Xr, Cc,YZe),(Xr,Cc,YZf),(Xr,Cc,YZg),(Xr,Cc,YZh),(Xr,Cc,YZi),(Xr,Cc,YZj),(Xr,Cc,YZk), (Xr,Cc,YZl),(Xr,Co,YZm),(Xr,Cc,YZn),(Xr,Cc,YZo),(Xr,Cc,YZp),(Xr,Cc,YZq),(Xr, Cc,YZr),(Xr,Cc,YZs),(Xr,Cc,YZt),(Xr,Cc,YZu),(Xr,Cc,YZv),(Xr,Cc,YZw),(Xr,Cc,YZx), (Xr,Cc,YZy),(Xr,Cc,YZz),(Xr,Cc,YZaa),(Xr,Cc,YZab),(Xr,Cc,YZac),(Xr,Cc,YZad), (Xr,Cc,YZae),(Xr,Cc,YZaf),(Xr,Cc,YZag),(Xr,Cc,YZah),(Xr,Cc,YZai),(Xr,Cc,YZaj), (Xr,Cc,YZak),(Xr,Cc,YZal),(Xr,Cc,YZam),(Xr,Cc,YZan),(Xr,Cc,YZao),(Xr,Cc,YZap), (Xr,Cc,YZaq),(Xr,Cc,YZar),(Xr,Cc,YZas),(Xr,Cc,YZat),(Xr,Cc,YZau),(Xr,Cc,YZav), (Xr,Cc,YZaw),(Xr,Cc,YZax),(Xr,Cc,YZay),(Xr,Cc,YZaz),(Xr,Cc,YZba),(Xr,Cc,YZbb), (Xr,Cc,YZbc),(Xr,Cc,YZbd),(Xr,Cc,YZbe),(Xr,Cc,YZbf),(Xr,Cc,YZbg),(Xr,Cc,YZbh), (Xr,Cc,YZbi),(Xr,Cc,YZbj),(Xr,Cc,YZbk),(Xr,Cc,YZbl),(Xr,Cc,YZbm),(Xr,Cc, YZbn),(Xr,Cc,YZbo),(Xr,Cc,YZbp),(Xr,Cc,YZbq),(Xr,Cc,YZbr),(Xr,Cc,YZbs),(Xr,Cc, YZbt),(Xr,Cc,YZbu),(Xr,Cc,YZbv),(Xr,Cc,YZbw),(Xr,Cc,YZbx),(Xr,Cc,YZby),(Xr, Cc,YZbz),(Xr,Cc,YZca),(Xr,Cc,YZcb),(Xr,Cc,YZcc),(Xr,Cc,YZcd),(Xr,Cc,YZce),(Xr, Cc,YZcf),(Xr,Cc,YZcg),(Xr,Cc,YZch),(Xr,Cc,YZci),(Xr,Cc,YZcj),(Xr,Cc,YZck),(Xr, Cc,YZcl),(Xr,Cc,YZcm),(Xr,Cc,YZcn),(Xr,Cc,YZco),(Xr,Cc,YZcp),(Xr,Cc,YZcq),(Xr, Cc,YZcr),(Xr,Cc,YZcs),(Xr,Cc,YZct),(Xr,Cc,YZcu),(Xr,Cc,YZcv),(Xr,Cc,YZcw),(Xr, Cc,YZcx),(Xr,Cc,YZcy),(Xr,Cc,YZcz),(Xr,Cc,YZda),(Xr,Cc,YZdb),(Xr,Cc,YZdc),(Xr, Cc,YZdd),(Xr,Cc,YZde),(Xr,Cc,YZdf),(Xr,Cc,YZdg),(Xr,Cc,YZdh),(Xr,Cc,YZdi),(Xr, Cc,YZdj),(Xr,Cc,YZdk),(Xr,Cc,YZdl),(Xr,Cc,YZdm),(Xr,Cc,YZdn),(Xr,Cc,YZdo), (Xr,Cc,YZdp),(Xr,Cc,YZdq),(Xr,Cc,YZdr),(Xr,Cc,YZds),(Xr,Cc,YZdt),(Xr,Cc,YZdu), (Xr,Cc,YZdv),(Xr,Cc,YZdw),(Xr,Cc,YZdx),(Xr,Cc,YZdy),(Xr,Cc,YZdz),(Xr,Cc,YZea), (Xr,Cc,YZeb),(Xr,Cc,YZec),(Xr,Cc,YZed),(Xr,Cc,YZee),(Xr,Cc,YZef),(Xr,Cc,YZeg), (Xr,Cc,YZeh),(Xr,Cc,YZei),(Xr,Cc,YZej),(Xr,Cc,YZek),(Xr,Cc,YZel),(Xr,Cc,YZem), (Xr,Cc,YZen),(Xr,Cc,YZeo),(Xr,Cc,YZep),(Xr,Cc,YZeq),(Xr,Cc,YZer),(Xr,Cc,YZes), (Xr,Cc,YZet),(Xr,Cc,YZeu),(Xr,Cc,YZev),(Xr,Cc,YZew),(Xr,Cc,YZex),(Xr,Cc,YZey), (Xr,Cc,YZez),(Xr,Cc,YZfa),(Xr,Cc,YZfb),(Xr,Cc,YZfc),(Xr,Cc,YZfd),(Xr,Cc,YZfe), (Xr,Cc,YZff),(Xr,Cc,YZfg),(Xr,Cc,YZfh),(Xr,Cc,YZfi),(Xr,Cc,YZfj),(Xr,Cc,YZfk),(Xr, Cc,YZfl),(Xr,Cc,YZfm),(Xr,Cc,YZfn),(Xr,Cc,YZfo),(Xr,Cc,YZfp),(Xr,Cc,YZfq),(Xr, Cc,YZfr),(Xr,Cc,YZfs),(Xr,Cc,YZft),(Xr,Cc,YZfu),(Xr,Cc,YZfv),(Xr,Cc,YZfw),(Xr, Cc,YZfx),(Xr,Cc,YZfy),(Xr,Cc,YZfz),(Xr,Cc,YZga),(Xr,Cc,YZgb),(Xr,Cc,YZgc),(Xr, Cc,YZgd),(Xr,Cc,YZge),(Xr,Cc,YZgf),(Xr,Cc,YZgg),(Xr,Cc,YZgh),(Xr,Cc,YZgi),(Xr, Cc,YZgj),(Xr,Cc,YZgk),(Xr,Cc,YZgl),(Xr,Cc,YZgm),(Xr,Cc,YZgn),(Xr,Cc,YZgo), (Xr,Cc,YZgp),(Xr,Cc,YZgq),(Xr,Cc,YZgr),(Xr,Cc,YZgs),(Xr,Cc,YZgt),(Xr,Cd,YZa),(Xr, Cd,YZb),(Xr,Cd,YZc),(Xr,Cd,YZd),(Xr,Cd,YZe),(Xr,Cd,YZf),(Xr,Cd,YZg),(Xr,Cd, YZh),(Xr,Cd,YZi),(Xr,Cd,YZj),(Xr,Cd,YZk),(Xr,Cd,YZl),(Xr,Cd,YZm),(Xr,Cd,YZn), (Xr,Cd,YZo),(Xr,Cd,YZp),(Xr,Cd,YZq),(Xr,Cd,YZr),(Xr,Cd,YZs),(Xr,Cd,YZt),(Xr, Cd,YZu),(Xr,Cd,YZv),(Xr,Cd,YZw),(Xr,Cd,YZx),(Xr,Cd,YZy),(Xr,Cd,YZz),(Xr,Cd, YZaa),(Xr,Cd,YZab),(Xr,Cd,YZac),(Xr,Cd,YZad),(Xr,Cd,YZae),(Xr,Cd,YZaf),(Xr,Cd, YZag),(Xr,Cd,YZah),(Xr,Cd,YZai),(Xr,Cd,YZaj),(Xr,Cd,YZak),(Xr,Cd,YZal),(Xr,Cd, YZam),(Xr,Cd,YZan),(Xr,Cd,YZao),(Xr,Cd,YZap),(Xr,Cd,YZaq),(Xr,Cd,YZar),(Xr, Cd,YZas),(Xr,Cd,YZat),(Xr,Cd,YZau),(Xr,Cd,YZav),(Xr,Cd,YZaw),(Xr,Cd,YZax),(Xr, Cd,YZay),(Xr,Cd,YZaz),(Xr,Cd,YZba),(Xr,Cd,YZbb),(Xr,Cd,YZbc),(Xr,Cd,YZbd),(Xr, Cd,YZbe),(Xr,Cd,YZbf),(Xr,Cd,YZbg),(Xr,Cd,YZbh),(Xr,Cd,YZbi),(Xr,Cd,YZbj), (Xr,Cd,YZbk),(Xr,Cd,YZbl),(Xr,Cd,YZbm),(Xr,Cd,YZbn),(Xr,Cd,YZbo),(Xr,Cd,YZbp), (Xr,Cd,YZbq),(Xr,Cd,YZbr),(Xr,Cd,YZbs),(Xr,Cd,YZbt),(Xr,Cd,YZbu),(Xr,Cd,YZbv), (Xr,Cd,YZbw),(Xr,Cd,YZbx),(Xr,Cd,YZby),(Xr,Cd,YZbz),(Xr,Cd,YZca),(Xr,Cd, YZcb),(Xr,Cd,YZcc),(Xr,Cd,YZcd),(Xr,Cd,YZce),(Xr,Cd,YZcf),(Xr,Cd,YZcg),(Xr,Cd, YZch),(Xr,Cd,YZci),(Xr,Cd,YZcj),(Xr,Cd,YZck),(Xr,Cd,YZcl),(Xr,Cd,YZcm),(Xr,Cd, YZcn),(Xr,Cd,YZco),(Xr,Cd,YZcp),(Xr,Cd,YZcq),(Xr,Cd,YZcr),(Xr,Cd,YZcs),(Xr, Cd,YZct),(Xr,Cd,YZcu),(Xr,Cd,YZcv),(Xr,Cd,YZcw),(Xr,Cd,YZcx),(Xr,Cd,YZcy),(Xr, Cd,YZcz),(Xr,Cd,YZda),(Xr,Cd,YZdb),(Xr,Cd,YZdc),(Xr,Cd,YZdd),(Xr,Cd,YZde),(Xr, Cd,YZdf),(Xr,Cd,YZdg),(Xr,Cd,YZdh),(Xr,Cd,YZdi),(Xr,Cd,YZdj),(Xr,Cd,YZdk), (Xr,Cd,YZdl),(Xr,Cd,YZdm),(Xr,Cd,YZdn),(Xr,Cd,YZdo),(Xr,Cd,YZdp),(Xr,Cd,YZdq), (Xr,Cd,YZdr),(Xr,Cd,YZds),(Xr,Cd,YZdt),(Xr,Cd,YZdu),(Xr,Cd,YZdv),(Xr,Cd,YZdw), (Xr,Cd,YZdx),(Xr,Cd,YZdy),(Xr,Cd,YZdz),(Xr,Cd,YZea),(Xr,Cd,YZeb),(Xr,Cd, YZec),(Xr,Cd,YZed),(Xr,Cd,YZee),(Xr,Cd,YZef),(Xr,Cd,YZeg),(Xr,Cd,YZeh),(Xr,Cd, YZei),(Xr,Cd,YZej),(Xr,Cd,YZek),(Xr,Cd,YZel),(Xr,Cd,YZem),(Xr,Cd,YZen),(Xr,Cd, YZeo),(Xr,Cd,YZep),(Xr,Cd,YZeq),(Xr,Cd,YZer),(Xr,Cd,YZes),(Xr,Cd,YZet),(Xr,Cd, YZeu),(Xr,Cd,YZev),(Xr,Cd,YZew),(Xr,Cd,YZex),(Xr,Cd,YZey),(Xr,Cd,YZez),(Xr, Cd,YZfa),(Xr,Cd,YZfb),(Xr,Cd,YZfc),(Xr,Cd,YZfd),(Xr,Cd,YZfe),(Xr,Cd,YZff),(Xr, Cd,YZfg),(Xr,Cd,YZfh),(Xr,Cd,YZfi),(Xr,Cd,YZfj),(Xr,Cd,YZfk),(Xr,Cd,YZfl),(Xr,Cd, YZfm),(Xr,Cd,YZfn),(Xr,Cd,YZfo),(Xr,Cd,YZfp),(Xr,Cd,YZfq),(Xr,Cd,YZfr),(Xr,Cd, YZfs),(Xr,Cd,YZft),(Xr,Cd,YZfu),(Xr,Cd,YZfv),(Xr,Cd,YZfw),(Xr,Cd,YZfx),(Xr,Cd, YZfy),(Xr,Cd,YZfz),(Xr,Cd,YZga),(Xr,Cd,YZgb),(Xr,Cd,YZgc),(Xr,Cd,YZgd),(Xr, Cd,YZge),(Xr,Cd,YZgf),(Xr,Cd,YZgg),(Xr,Cd,YZgh),(Xr,Cd,YZgi),(Xr,Cd,YZgj),(Xr, Cd,YZgk),(Xr,Cd,YZgl),(Xr,Cd,YZgm),(Xr,Cd,YZgn),(Xr,Cd,YZgo),(Xr,Cd,YZgp),(Xr, Cd,YZgq),(Xr,Cd,YZgr),(Xr,Cd,YZgs),(Xr,Cd,YZgt),(Xr,Ce,YZa),(Xr,Ce,YZb),(Xr, Ce,YZc),(Xr,Ce,YZd),(Xr,Ce,YZe),(Xr,Ce,YZf),(Xr,Ce,YZg),(Xr,Ce,YZh),(Xr,Ce,YZi), (XT,Ce,YZj),(Xr,Ce,YZk),(Xr,Ce,YZl),(Xr,Cc,YZm),(Xr,Ce,YZn),(Xr,Ce,YZo),(Xr, Ce,YZp),(Xr,Ce,YZq),(Xr,Ce,YZr),(Xr,Ce,YZs),(Xr,Ce,YZt),(Xr,Ce,YZu),(Xr,Ce,YZv), (Xr,Ce,YZw),(Xr,Ce,YZx),(Xr,Ce,YZy),(XCr,Ce,YZz),(Xr,Ce,YZaa),(Xr,Ce,YZab), (Xr,Ce,YZac),(Xr,Ce,YZad),(Xr,Ce,YZae),(Xr,Ce,YZaf),(Xr,Ce,YZag),(Xr,Ce,YZah), (Xr,Ce,YZai),(Xr,Ce,YZaj),(Xr,Ce,YZak),(Xr,Ce,YZal),(Xr,Ce,YZam),(Xr,Ce,YZan), (Xr,Ce,YZao),(Xr,Ce,YZap),(Xr,Ce,YZaq),(Xr,Ce,YZar),(Xr,Ce,YZas),(Xr,Ce,YZat), (Xr,Ce,YZau),(Xr,Ce,YZav),(Xr,Ce,YZaw),(Xr,Ce,YZax),(Xr,Ce,YZay),(Xr,Ce,YZaz), (Xr,Ce,YZba),(Xr,Ce,YZbb),(Xr,Ce,YZbc),(Xr,Ce,YZbd),(Xr,Ce,YZbe),(Xr,Ce,YZbf), (Xr,Ce,YZbg),(Xr,Ce,YZbh),(Xr,Ce,YZbi),(Xr,Ce,YZbj),(Xr,Ce,YZbk),(Xr,Ce,YZbl), (Xr,Ce,YZbm),(Xr,Ce,YZbn),(Xr,Ce,YZbo),(Xr,Ce,YZbp),(Xr,Ce,YZbq),(Xr,Ce,YZbr), (Xr,Ce,YZbs),(Xr,Ce,YZbt),(Xr,Ce,YZbu),(Xr,Ce,YZbv),(Xr,Ce,YZbw),(Xr,Ce, YZbx),(Xr,Ce,YZby),(Xr,Ce,YZbz),(Xr,Ce,YZca),(Xr,Ce,YZcb),(Xr,Ce,YZcc),(Xr,Ce, YZcd),(Xr,Ce,YZce),(Xr,Ce,YZcf),(Xr,Ce,YZcg),(Xr,Ce,YZch),(Xr,Ce,YZci),(Xr,Ce, YZcj),(Xr,Ce,YZck),(Xr,Ce,YZcl),(Xr,Ce,YZcm),(Xr,Ce,YZcn),(Xr,Ce,YZco),(Xr,Ce, YZcp),(Xr,Ce,YZcq),(Xr,Ce,YZcr),(Xr,Ce,YZcs),(Xr,Ce,YZct),(Xr,Ce,YZcu),(Xr,Ce, YZcv),(Xr,Ce,YZcw),(Xr,Ce,YZcx),(Xr,Ce,YZcy),(Xr,Ce,YZcz),(Xr,Ce,YZda),(Xr,Ce, YZdb),(Xr,Ce,YZdc),(Xr,Ce,YZdd),(Xr,Ce,YZde),(Xr,Ce,YZdf),(Xr,Ce,YZdg),(Xr, Ce,YZdh),(Xr,Ce,YZdi),(Xr,Ce,YZdj),(Xr,Ce,YZdk),(Xr,Ce,YZdl),(Xr,Ce,YZdm),(Xr, Ce,YZdn),(Xr,Ce,YZdo),(Xr,Ce,YZdp),(Xr,Ce,YZdq),(Xr,Ce,YZdr),(Xr,Ce,YZds),(Xr, Ce,YZdt),(Xr,Ce,YZdu),(Xr,Ce,YZdv),(Xr,Ce,YZdw),(Xr,Ce,YZdx),(Xr,Ce,YZdy),(Xr, Ce,YZdz),(Xr,Ce,YZea),(Xr,Ce,YZeb),(Xr,Ce,YZec),(Xr,Ce,YZed),(Xr,Ce,YZee),(Xr, Ce,YZef),(Xr,Ce,YZeg),(Xr,Ce,YZeh),(Xr,Ce,YZei),(Xr,Ce,YZej),(Xr,Ce,YZek),(Xr, Ce,YZel),(Xr,Ce,YZem),(Xr,Ce,YZen),(Xr,Ce,YZeo),(Xr,Ce,YZep),(Xr,Ce,YZeq), (Xr,Ce,YZer),(Xr,Ce,YZes),(Xr,Ce,YZet),(Xr,Ce,YZeu),(Xr,Ce,YZev),(Xr,Ce,YZew), (Xr,Ce,YZex),(Xr,Ce,YZey),(Xr,Ce,YZez),(Xr,Ce,YZfa),(Xr,Ce,YZfb),(Xr,Ce,YZfc),(Xr, Ce,YZfd),(Xr,Ce,YZfe),(Xr,Ce,YZff),(Xr,Ce,YZfg),(Xr,Ce,YZfh),(Xr,Ce,YZfi),(Xr, Ce,YZfj),(Xr,Ce,YZfk),(Xr,Ce,YZfl),(Xr,Ce,YZfm),(Xr,Ce,YZfn),(Xr,Ce,YZfo),(Xr, Ce,YZfp),(Xr,Ce,YZfq),(Xr,Ce,YZfr),(Xr,Ce,YZfs),(Xr,Ce,YZft),(Xr,Ce,YZfu),(Xr,Ce, YZfv),(Xr,Ce,YZfw),(Xr,Ce,YZfx),(Xr,Ce,YZfy),(Xr,Ce,YZfz),(Xr,Ce,YZga),(Xr,Ce, YZgb),(Xr,Ce,YZgc),(Xr,Ce,YZgd),(Xr,Ce,YZge),(Xr,Ce,YZgf),(Xr,Ce,YZgg),(Xr, Ce,YZgh),(Xr,Ce,YZgi),(Xr,Ce,YZgj),(Xr,Ce,YZgk),(Xr,Ce,YZgl),(Xr,Ce,YZgm),(Xr, Ce,YZgn),(Xr,Ce,YZgo),(Xr,Ce,YZgp),(Xr,Ce,YZgq),(Xr,Ce,YZgr),(Xr,Ce,YZgs),(Xr, Ce,YZgt),(Xr,Cf,YZa),(Xr,Cf,YZb),(Xr,Cf,YZc),(Xr,Cf,YZd),(Xr,Cf,YZe),(Xr,Cf,YZf), (Xr,Cf,YZg),(Xr,Cf,YZh),(Xr,Cf,YZi),(Xr,Cf,YZj),(Xr,Cf,YZk),(Xr,Cf,YZl),(Xr,Cf, YZm),(Xr,Cf,YZn),(Xr,Cf,YZo),(Xr,Cf,YZp),(Xr,Cf,YZq),(Xr,Cf,YZr),(Xr,Cf,YZs), (Xr,Cf,YZt),(Xr,Cf,YZu),(Xr,Cf,YZv),(Xr,Cf,YZw),(Xr,Cf,YZx),(Xr,Cf,YZy),(Xr,Cf, YZz),(Xr,Cf,YZaa),(Xr,Cf,YZab),(Xr,Cf,YZac),(Xr,Cf,YZad),(Xr,Cf,YZae),(Xr,Cf,YZaf), (Xr,Cf,YZag),(Xr,Cf,YZah),(Xr,Cf,YZai),(Xr,Cf,YZaj),(Xr,Cf,YZak),(Xr,Cf,YZal), (Xr,Cf,YZam),(Xr,Cf,YZan),(Xr,Cf,YZao),(Xr,Cf,YZap),(Xr,Cf,YZaq),(Xr,Cf,YZar), (Xr,Cf,YZas),(Xr,Cf,YZat),(Xr,Cf,YZau),(Xr,Cf,YZav),(Xr,Cf,YZaw),(Xr,Cf,YZax), (Xr,Cf,YZay),(Xr,Cf,YZaz),(Xr,Cf,YZba),(Xr,Cf,YZbb),(Xr,Cf,YZbc),(Xr,Cf,YZbd),(Xr, Cf,YZbe),(Xr,Cf,YZbf),(Xr,Cf,YZbg),(Xr,Cf,YZbh),(Xr,Cf,YZbi),(Xr,Cf,YZbj),(Xr, Cf,YZbk),(Xr,Cf,YZbl),(Xr,Cf,YZbm),(Xr,Cf,YZbn),(Xr,Cf,YZbo),(Xr,Cf,YZbp),(Xr, Cf,YZbq),(Xr,Cf,YZbr),(Xr,Cf,YZbs),(Xr,Cf,YZbt),(Xr,Cf,YZbu),(Xr,Cf,YZbv),(Xr, Cf,YZbw),(Xr,Cf,YZbx),(Xr,Cf,YZby),(Xr,Cf,YZbz),(Xr,Cf,YZca),(Xr,Cf,YZcb),(Xr, Cf,YZcc),(Xr,Cf,YZcd),(Xr,Cf,YZce),(Xr,Cf,YZcf),(Xr,Cf,YZcg),(Xr,Cf,YZch),(Xr,Cf, YZci),(Xr,Cf,YZcj),(Xr,Cf,YZck),(Xr,Cf,YZcl),(Xr,Cf,YZcm),(Xr,Cf,YZcn),(Xr,Cf, YZco),(Xr,Cf,YZcp),(Xr,Cf,YZcq),(Xr,Cf,YZcr),(Xr,Cf,YZcs),(Xr,Cf,YZct),(Xr,Cf,YZcu), (Xr,Cf,YZcv),(Xr,Cf,YZcw),(Xr,Cf,YZcx),(Xr,Cf,YZcy),(Xr,Cf,YZcz),(Xr,Cf,YZda), (Xr,Cf,YZdb),(Xr,Cf,YZdc),(Xr,Cf,YZdd),(Xr,Cf,YZde),(Xr,Cf,YZdf),(Xr,Cf,YZdg), (Xr,Cf,YZdh),(Xr,Cf,YZdi),(Xr,Cf,YZdj),(Xr,Cf,YZdk),(Xr,Cf,YZdl),(Xr,Cf,YZdm), (Xr,Cf,YZdn),(Xr,Cf,YZdo),(Xr,Cf,YZdp),(Xr,Cf,YZdq),(Xr,Cf,YZdr),(Xr,Cf,YZds), (Xr,Cf,YZdt),(Xr,Cf,YZdu),(Xr,Cf,YZdv),(Xr,Cf,YZdw),(Xr,Cf,YZdx),(Xr,Cf,YZdy), (Xr,Cf,YZdz),(Xr,Cf,YZea),(Xr,Cf,YZeb),(Xr,Cf,YZec),(Xr,Cf,YZed),(Xr,Cf,YZee), (Xr,Cf,YZef),(Xr,Cf,YZeg),(Xr,Cf,YZeh),(Xr,Cf,YZei),(Xr,Cf,YZej),(Xr,Cf,YZek), (Xr,Cf,YZel),(Xr,Cf,YZem),(Xr,Cf,YZen),(Xr,Cf,YZeo),(Xr,Cf,YZep),(Xr,Cf,YZeq),(Xr, Cf,YZer),(Xr,Cf,YZes),(Xr,Cf,YZet),(Xr,Cf,YZeu),(Xr,Cf,YZev),(Xr,Cf,YZew),(Xr, Cf,YZex),(Xr,Cf,YZey),(Xr,Cf,YZez),(Xr,Cf,YZfa),(Xr,Cf,YZfb),(Xr,Cf,YZfc),(Xr, Cf,YZfd),(Xr,Cf,YZfe),(Xr,Cf,YZff),(Xr,Cf,YZfg),(Xr,Cf,YZfh),(Xr,Cf,YZfi),(Xr,Cf, YZfj),(Xr,Cf,YZfk),(Xr,Cf,YZfl),(Xr,Cf,YZfm),(Xr,Cf,YZfn),(Xr,Cf,YZfo),(Xr,Cf,YZfp), (Xr,Cf,YZfq),(Xr,Cf,YZfr),(Xr,Cf,YZfs),(Xr,Cf,YZft),(Xr,Cf,YZfu),(Xr,Cf,YZfv), (Xr,Cf,YZfw),(Xr,Cf,YZfx),(Xr,Cf,YZfy),(Xr,Cf,YZfz),(Xr,Cf,YZga),(Xr,Cf,YZgb),(Xr, Cf,YZgc),(Xr,Cf,YZgd),(Xr,Cf,YZge),(Xr,Cf,YZgf),(Xr,Cf,YZgg),(Xr,Cf,YZgh),(Xr, Cf,YZgi),(Xr,Cf,YZgj),(Xr,Cf,YZgk),(Xr,Cf,YZgl),(Xr,Cf,YZgm),(Xr,Cf,YZgn),(Xr, Cf,YZgo),(Xr,Cf,YZgp),(Xr,Cf,YZgq),(Xr,Cf,YZgr),(Xr,Cf,YZgs),(Xr,Cf,YZgt), (Xs,Ca,YZa),(Xs,Ca,YZb),(Xs,Ca,YZc),(Xs,Ca,YZd),(Xs,Ca,YZe),(Xs,Ca,YZf),(Xs,Ca, YZg),(Xs,Ca,YZh),(Xs,Ca,YZi),(Xs,Ca,YZj),(Xs,Ca,YZk),(Xs,Ca,YZl),(Xs,Ca,YZm), (Xs,Ca,YZn),(Xs,Ca,YZo),(Xs,Ca,YZp),(Xs,Ca,YZq),(Xs,Ca,YZr),(Xs,Ca,YZs),(Xs, Ca,YZt),(Xs,Ca,YZu),(Xs,Ca,YZv),(Xs,Ca,YZw),(Xs,Ca,YZx),(Xs,Ca,YZy),(Xs,Ca,YZz), (Xs,Ca,YZaa),(Xs,Ca,YZab),(Xs,Ca,YZac),(Xs,Ca,YZad),(Xs,Ca,YZae),(Xs,Ca,YZaf), (Xs,Ca,YZag),(Xs,Ca,YZah),(Xs,Ca,YZai),(Xs,Ca,YZaj),(Xs,Ca,YZak),(Xs,Ca,YZal), (Xs,Ca,YZam),(Xs,Ca,YZan),(Xs,Ca,YZao),(Xs,Ca,YZap),(Xs,Ca,YZaq),(Xs,Ca, YZar),(Xs,Ca,YZas),(Xs,Ca,YZat),(Xs,Ca,YZau),(Xs,Ca,YZav),(Xs,Ca,YZaw),(Xs,Ca, YZax),(Xs,Ca,YZay),(Xs,Ca,YZaz),(Xs,Ca,YZba),(Xs,Ca,YZbb),(Xs,Ca,YZbc),(Xs, Ca,YZbd),(Xs,Ca,YZbe),(Xs,Ca,YZbf),(Xs,Ca,YZbg),(Xs,Ca,YZbh),(Xs,Ca,YZbi),(Xs, Ca,YZbj),(Xs,Ca,YZbk),(Xs,Ca,YZbl),(Xs,Ca,YZbm),(Xs,Ca,YZbn),(Xs,Ca,YZbo),(Xs, Ca,YZbp),(Xs,Ca,YZbq),(Xs,Ca,YZbr),(Xs,Ca,YZbs),(Xs,Ca,YZbt),(Xs,Ca,YZbu), (Xs,Ca,YZbv),(Xs,Ca,YZbw),(Xs,Ca,YZbx),(Xs,Ca,YZby),(Xs,Ca,YZbz),(Xs,Ca,YZca), (Xs,Ca,YZcb),(Xs,Ca,YZcc),(Xs,Ca,YZcd),(Xs,Ca,YZce),(Xs,Ca,YZcf),(Xs,Ca,YZcg), (Xs,Ca,YZch),(Xs,Ca,YZci),(Xs,Ca,YZcj),(Xs,Ca,YZck),(Xs,Ca,YZcl),(Xs,Ca,YZcm), (Xs,Ca,YZcn),(Xs,Ca,YZco),(Xs,Ca,YZcp),(Xs,Ca,YZcq),(Xs,Ca,YZcr),(Xs,Ca,YZcs), (Xs,Ca,YZct),(Xs,Ca,YZcu),(Xs,Ca,YZcv),(Xs,Ca,YZcw),(Xs,Ca,YZcx),(Xs,Ca, YZcy),(Xs,Ca,YZcz),(Xs,Ca,YZda),(Xs,Ca,YZdb),(Xs,Ca,YZdc),(Xs,Ca,YZdd),(Xs,Ca, YZde),(Xs,Ca,YZdf),(Xs,Ca,YZdg),(Xs,Ca,YZdh),(Xs,Ca,YZdi),(Xs,Ca,YZdj),(Xs, Ca,YZdk),(Xs,Ca,YZdl),(Xs,Ca,YZdm),(Xs,Ca,YZdn),(Xs,Ca,YZdo),(Xs,Ca,YZdp),(Xs, Ca,YZdq),(Xs,Ca,YZdr),(Xs,Ca,YZds),(Xs,Ca,YZdt),(Xs,Ca,YZdu),(Xs,Ca,YZdv), (Xs,Ca,YZdw),(Xs,Ca,YZdx),(Xs,Ca,YZdy),(Xs,Ca,YZdz),(Xs,Ca,YZea),(Xs,Ca,YZeb), (Xs,Ca,YZec),(Xs,Ca,YZed),(Xs,Ca,YZee),(Xs,Ca,YZef),(Xs,Ca,YZeg),(Xs,Ca,YZeh), (Xs,Ca,YZei),(Xs,Ca,YZej),(Xs,Ca,YZek),(Xs,Ca,YZel),(Xs,Ca,YZem),(Xs,Ca,YZen), (Xs,Ca,YZeo),(Xs,Ca,YZep),(Xs,Ca,YZeq),(Xs,Ca,YZer),(Xs,Ca,YZes),(Xs,Ca, YZet),(Xs,Ca,YZeu),(Xs,Ca,YZev),(Xs,Ca,YZew),(Xs,Ca,YZex),(Xs,Ca,YZey),(Xs,Ca, YZez),(Xs,Ca,YZfa),(Xs,Ca,YZfb),(Xs,Ca,YZfc),(Xs,Ca,YZfd),(Xs,Ca,YZfe),(Xs,Ca, YZff),(Xs,Ca,YZfg),(Xs,Ca,YZfh),(Xs,Ca,YZfi),(Xs,Ca,YZfj),(Xs,Ca,YZfk),(Xs,Ca, YZfl),(Xs,Ca,YZfm),(Xs,Ca,YZfn),(Xs,Ca,YZfo),(Xs,Ca,YZfp),(Xs,Ca,YZfq),(Xs,Ca, YZfr),(Xs,Ca,YZfs),(Xs,Ca,YZft),(Xs,Ca,YZfu),(Xs,Ca,YZfv),(Xs,Ca,YZfw),(Xs,Ca, YZfx),(Xs,Ca,YZfy),(Xs,Ca,YZfz),(Xs,Ca,YZga),(Xs,Ca,YZgb),(Xs,Ca,YZgc),(Xs,Ca, YZgd),(Xs,Ca,YZge),(Xs,Ca,YZgf),(Xs,Ca,YZgg),(Xs,Ca,YZgh),(Xs,Ca,YZgi),(Xs,Ca, YZgj),(Xs,Ca,YZgk),(Xs,Ca,YZgl),(Xs,Ca,YZgm),(Xs,Ca,YZgn),(Xs,Ca,YZgo),(Xs, Ca,YZgp),(Xs,Ca,YZgq),(Xs,Ca,YZgr),(Xs,Ca,YZgs),(Xs,Ca,YZgt),(Xs,Cb,YZa),(Xs, Cb,YZb),(Xs,Cb,YZc),(Xs,Cb,YZd),(Xs,Cb,YZe),(Xs,Cb,YZf),(Xs,Cb,YZg),(Xs,Cb, YZh),(Xs,Cb,YZi),(Xs,Cb,YZj),(Xs,Cb,YZk),(Xs,Cb,YZl),(Xs,Cb,YZm),(Xs,Cb,YZn), (Xs,Cb,YZo),(Xs,Cb,YZp),(Xs,Cb,YZq),(Xs,Cb,YZr),(Xs,Cb,YZs),(Xs,Cb,YZt),(Xs, Cb,YZu),(Xs,Cb,YZv),(Xs,Cb,YZw),(Xs,Cb,YZx),(Xs,Cb,YZy),(Xs,Cb,YZz),(Xs,Cb, YZaa),(Xs,Cb,YZab),(Xs,Cb,YZac),(Xs,Cb,YZad),(Xs,Cb,YZae),(Xs,Cb,YZaf),(Xs,Cb, YZag),(Xs,Cb,YZah),(Xs,Cb,YZai),(Xs,Cb,YZaj),(Xs,Cb,YZak),(Xs,Cb,YZal),(Xs, Cb,YZam),(Xs,Cb,YZan),(Xs,Cb,YZao),(Xs,Cb,YZap),(Xs,Cb,YZaq),(Xs,Cb,YZar),(Xs, Cb,YZas),(Xs,Cb,YZat),(Xs,Cb,YZau),(Xs,Cb,YZav),(Xs,Cb,YZaw),(Xs,Cb,YZax), (Xs,Cb,YZay),(Xs,Cb,YZaz),(Xs,Cb,YZba),(Xs,Cb,YZbb),(Xs,Cb,YZbc),(Xs,Cb,YZbd), (Xs,Cb,YZbe),(Xs,Cb,YZbf),(Xs,Cb,YZbg),(Xs,Cb,YZbh),(Xs,Cb,YZbi),(Xs,Cb, YZbj),(Xs,Cb,YZbk),(Xs,Cb,YZbl),(Xs,Cb,YZbm),(Xs,Cb,YZbn),(Xs,Cb,YZbo),(Xs, Cb,YZbp),(Xs,Cb,YZbq),(Xs,Cb,YZbr),(Xs,Cb,YZbs),(Xs,Cb,YZbt),(Xs,Cb,YZbu),(Xs, Cb,YZbv),(Xs,Cb,YZbw),(Xs,Cb,YZbx),(Xs,Cb,YZby),(Xs,Cb,YZbz),(Xs,Cb,YZca), (Xs,Cb,YZcb),(Xs,Cb,YZcc),(Xs,Cb,YZcd),(Xs,Cb,YZce),(Xs,Cb,YZcf),(Xs,Cb,YZcg), (Xs,Cb,YZch),(Xs,Cb,YZci),(Xs,Cb,YZcj),(Xs,Cb,YZck),(Xs,Cb,YZcl),(Xs,Cb, YZcm),(Xs,Cb,YZcn),(Xs,Cb,YZco),(Xs,Cb,YZcp),(Xs,Cb,YZcq),(Xs,Cb,YZcr),(Xs, Cb,YZcs),(Xs,Cb,YZct),(Xs,Cb,YZcu),(Xs,Cb,YZcv),(Xs,Cb,YZcw),(Xs,Cb,YZcx),(Xs, Cb,YZcy),(Xs,Cb,YZcz),(Xs,Cb,YZda),(Xs,Cb,YZdb),(Xs,Cb,YZdc),(Xs,Cb,YZdd), (Xs,Cb,YZde),(Xs,Cb,YZdf),(Xs,Cb,YZdg),(Xs,Cb,Y.Zdh),(Xs,Cb,YZdi),(Xs,Cb,YZdj), (Xs,Cb,YZdk),(Xs,Cb,YZdl),(Xs,Cb,YZdm),(Xs,Cb,YZdn),(Xs,Cb,YZdo),(Xs,Cb, YZdp),(Xs,Cb,YZdq),(Xs,Cb,YZdr),(Xs,Cb,YZds),(Xs,Cb,YZdt),(Xs,Cb,YZdu),(Xs,Cb, YZdv),(Xs,Cb,YZdw),(Xs,Cb,YZdx),(Xs,Cb,YZdy),(Xs,Cb,YZdz),(Xs,Cb,YZea),(Xs, Cb,YZeb),(Xs,Cb,YZec),(Xs,Cb,YZed),(Xs,Cb,YZee),(Xs,Cb,YZef),(Xs,Cb,YZeg),(Xs, Cb,YZeh),(Xs,Cb,YZei),(Xs,Cb,YZej),(Xs,Cb,YZek),(Xs,Cb,YZel),(Xs,Cb,YZem), (Xs,Cb,YZen),(Xs,Cb,YZeo),(Xs,Cb,YZep),(Xs,Cb,YZeq),(Xs,Cb,YZer),(Xs,Cb,YZes), (Xs,Cb,YZet),(Xs,Cb,YZeu),(Xs,Cb,YZev),(Xs,Cb,YZew),(Xs,Cb,YZex),(Xs,Cb,YZey), (Xs,cb,YZez),(Xs,Cb,YZfa),(Xs,Cb,YZfb),(Xs,Cb,YZfc),(Xs,Cb,YZfd),(Xs,Cb, YZfe),(Xs,Cb,YZff),(Xs,Cb,YZfg),(Xs,Cb,YZfh),(Xs,Cb,YZfi),(Xs,Cb,YZfj),(Xs,Cb, YZfk),(Xs,Cb,YZfl),(Xs,Cb,YZfm),(Xs,Cb,YZfn),(Xs,Cb,YZfo),(Xs,Cb,YZfp),(Xs,Cb, YZfq),(Xs,Cb,YZfr),(Xs,Cb,YZfs),(Xs,Cb,YZft),(Xs,Cb,YZu),(Xs,Cb,YZfv),(Xs,Cb, YZfw),(Xs,Cb,YZfx),(Xs,Cb,YZfy),(Xs,Cb,YZfz),(Xs,Cb,YZga),(Xs,Cb,YZgb),(Xs,Cb, YZgc),(Xs,Cb,YZgd),(Xs,Cb,YZge),(Xs,Cb,YZgf),(Xs,Cb,YZgg),(Xs,Cb,YZgh),(Xs, Cb,YZgi),(Xs,Cb,YZgj),(Xs,Cb,YZgk),(Xs,Cb,YZgl),(Xs,Cb,YZgm),(Xs,Cb,YZgn),(Xs, Cb,YZgo),(Xs,Cb,YZgp),(Xs,Cb,YZgq),(Xs,Cb,YZgr),(Xs,Cb,YZgs),(Xs,Cb,YZgt), (Xs,Cc,YZa),(Xs,Cc,YZb),(Xs,Cc,YZc),(Xs,Cc,YZd),(Xs,Cc,YZe),(Xs,Cc,YZf),(Xs, Cc,YZg),(Xs,Cc,YZh),(Xs,Cc,YZi),(Xs,Cc,YZj),(Xs,Cc,YZk),(Xs,Cc,YlI),(Xs,Cc,YZm), (Xs,Cc,YZn),(Xs,Cc,YZo),(Xs,Cc,YZp),(Xs,Cc,YZq),(Xs,Cc,YZr),(Xs,Cc,YZs),(Xs, Cc,YZt),(Xs,Cc,YZu),(Xs,Cc,YZv),(Xs,Cc,YZw),(Xs,cC,YZx),(Xs,Cc,YZy),(Xs,Cc, YZz),(Xs,Cc,YZaa),(Xs,Cc,YZab),(Xs,Cc,YZac),(Xs,Cc,YZad),(Xs,Cc,YZae),(Xs,Ce, YZaf),(Xs,Cc,YZag),(Xs,Cc,YZah),(Xs,Cc,YZai),(Xs,Cc,YZaj),(Xs,Cc,YZak),(Xs,Cc, YZal),(Xs,Cc,YZam),(Xs,Cc,YZan),(Xs,Cc,YZao),(Xs,Cc,YZap),(Xs,Cc,YZaq),(Xs,Cc, YZar),(Xs,Cc,YZas),(Xs,Cc,YZat),(Xs,Cc,YZau),(Xs,Cc,YZav),(Xs,Cc,YZaw),(Xs, Cc,YZax),(Xs,Cc,YZay),(Xs,Cc,YZaz),(Xs,Cc,YZba),(Xs,Cc,YZbb),(Xs,Cc,YZbc),(Xs, Cc,YZbd),(Xs,Cc,YZbe),(Xs,Cc,YZbf),(Xs,Cc,YZbg),(Xs,Cc,YZbh),(Xs,Cc,YZbi),(Xs, Cc,YZbj),(Xs,Cc,YZbk),(Xs,Cc,YZbl),(Xs,Cc,YZbm),(Xs,Cc,YZbn),(Xs,Cc,YZbo), (Xs,Cc,YZbp),(Xs,Cc,YZbq),(Xs,Cc,YZbr),(Xs,Cc,YZbs),(Xs,Cc,YZbt),(Xs,Cc,YZbu), (Xs,Cc,YZbv),(Xs,Cc,YZbw),(Xs,Cc,YZbx),(Xs,Cc,YZby),(Xs,Cc,YZbz),(Xs,Cc,YZca), (Xs,Ce,YZcb),(Xs,Cc,YZcc),(Xs,Cc,YZcd),(Xs,Ce,YZce),(Xs,Cc,YZcf),(Xs,Ce, YZcg),(Xs,Cc,YZch),(Xs,Cc,YZci),(Xs,Cc,YZcj),(Xs,Cc,YZck),(Xs,Cc,YZcl),(Xs,Cc, YZcm),(Xs,Cc,YZcn),(Xs,Cc,YZco),(Xs,Cc,YZcp),(Xs,Cc,YZcq),(Xs,Cc,YZcr),(Xs,Cc, YZcs),(Xs,Cc,YZct),(Xs,Cc,YZcu),(Xs,Cc,YZcv),(Xs,Cc,YZcw),(Xs,Cc,YZcx),(Xs, Cc,YZcy),(Xs,Cc,YZcz),(Xs,Cc,YZda),(Xs,Cc,YZdb),(Xs,Cc,YZdc),(Xs,Cc,YZdd),(Xs, Cc,YZde),(Xs,Cc,YZdf),(Xs,Cc,YZdg),(Xs,Cc,YZdh),(Xs,Cc,YZdi),(Xs,Cc,YZdj),(Xs, Cc,YZdk),(Xs,Cc,YZdl),(Xs,Cc,YZdm),(Xs,Cc,YZdn),(Xs,Cc,YZdo),(Xs,Cc,YZdp), (Xs,Cc,YZdq),(Xs,Cc,YZdr),(Xs,Cc,YZds),(Xs,Cc,YZdt),(Xs,Cc,YZdu),(Xs,Cc,YZdv), (Xs,Cc,YZdw),(Xs,Cc,YZdx),(Xs,Cc,YZdy),(Xs,Cc,YZdz),(Xs,Cc,YZea),(Xs,Cc,YZeb), (Xs,Cc,YZec),(Xs,Cc,YZed),(Xs,Cc,YZee),(Xs,Cc,YZef),(Xs,Cc,YZeg),(Xs,Cc, YZeh),(Xs,Cc,YZei),(Xs,Cc,YZej),(Xs,Cc,YZek),(Xs,Cc,YZel),(Xs,Cc,YZem),(Xs,Cc, YZen),(Xs,Cc,YZeo),(Xs,Cc,YZep),(Xs,Cc,YZeq),(Xs,Cc,YZer),(Xs,Cc,YZes),(Xs,Cc, YZet),(Xs,Cc,YZeu),(Xs,Cc,YZev),(Xs,Cc,YZew),(Xs,Cc,YZex),(Xs,Cc,YZey),(Xs, Cc,YZez),(Xs,Cc,YZfa),(Xs,Cc,YZfb),(Xs,Cc,YZfc),(Xs,Cc,YZfd),(Xs,Cc,YZfe),(Xs, Cc,YZff),(Xs,Cc,YZfg),(Xs,Cc,YZfh),(Xs,Cc,YZfi),(Xs,Cc,YZfj),(Xs,Cc,YZfk),(Xs,Cc, YZfl),(Xs,Cc,YZfm),(Xs,Cc,YZfn),(Xs,Cc,YZfo),(Xs,Cc,YZfp),(Ns,Cc,YZfq),(Xs,Cc, YZfr),(Xs,Cc,YZfs),(Xs,Cc,YZft),(Xs,Cc,YZfu),(Xs,Cc,YZfv),(Xs,Cc,YZfw),(Xs,Cc, YZfx),(Xs,Cc,YZfy),(Xs,Cc,YZfz),(Xs,Cc,YZga),(Xs,Cc,YZgb),(Xs,Cc,YZgc),(Xs,Cc, YZgd),(Xs,Cc,YZge),(Xs,Cc,YZgf),(Xs,Cc,YZgg),(Xs,Cc,YZgh),(Xs,Cc,YZgi),(Xs, Cc,YZgj),(Xs,Cc,YZgk),(Xs,Cc,YZgl),(Xs,Cc,YZgm),(Xs,Cc,YZgn),(Xs,Cc,YZgo),(Xs, Cc,YZgp),(Xs,Cc,YZgq),(Xs,Cc,YZgr),(Xs,Cc,YZgs),(Xs,Cc,YZgt),(Xs,Cd,YZa),(Xs, Cd,YZb),(Xs,Cd,YZc),(Xs,Cd,YZd),(Xs,Cd,YZe),(Xs,Cd,YZf),(Xs,Cd,YZg),(Xs,Cd, YZh),(Xs,Cd,YZi),(Xs,Cd,YZj),(Xs,Cd,YZk),(Xs,Cd,YZl),(Xs,Cd,YZm),(Xs,Cd,YZn), (Xs,Cd,YZo),(Xs,Cd,YZp),(Xs,Cd,YZq),(Xs,Cd,YZr),(Xs,Cd,YZs),(Xs,Cd,YZt),(Xs, Cd,YZu),(Xs,Cd,YZv),(Xs,Cd,YZw),(Xs,Cd,YZx),(Xs,Cd,YZy),(Xs,Cd,YZz),(Xs,Cd, YZaa),(Xs,Cd,YZab),(Xs,Cd,YZac),(Xs,Cd,YZad),(Xs,Cd,YZae),(Xs,Cd,YZaf),(Xs, Cd,YZag),(Xs,Cd,YZah),(Xs,Cd,YZai),(Xs,Cd,YZaj),(Xs,Cd,YZak),(Xs,Cd,YZal),(Xs, Cd,YZam),(Xs,Cd,YZan),(Xs,Cd,YZao),(Xs,Cd,YZap),(Xs,Cd,YZaq),(Xs,Cd,YZar),(Xs, Cd,YZas),(Xs,Cd,YZat),(Xs,Cd,YZau),(Xs,Cd,YZav),(Xs,Cd,YZaw),(Xs,Cd,YZax), (Xs,Cd,YZay),(Xs,Cd,YZaz),(Xs,Cd,YZba),(Xs,Cd,YZbb),(Xs,Cd,YZbc),(Xs,Cd,YZbd), (Xs,Cd,YZbe),(Xs,Cd,YZbf),(Xs,Cd,YZbg),(Xs,Cd,YZbh),(Xs,Cd,YZbi),(Xs,Cd, YZbj),(Xs,Cd,YZbk),(Xs,Cd,YZbl),(Xs,Cd,YZbm),(Xs,Cd,YZbn),(Xs,Cd,YZbo),(Xs, Cd,YZbp),(Xs,Cd,YZbq),(Xs,Cd,YZbr),(Xs,Cd,YZbs),(Xs,Cd,YZbt),(Xs,Cd,YZbu),(Xs, Cd,YZbv),(Xs,Cd,YZbw),(Xs,Cd,YZbx),(Xs,Cd,YZby),(Xs,Cd,YZbz),(Xs,Cd,YZca), (Xs,Cd,YZcb),(Xs,Cd,YZcc),(Xs,Cd,YZcd),(Xs,Cd,YZce),(Xs,Cd,YZcf),(Xs,Cd,YZcg), (Xs,Cd,YZch),(Xs,Cd,YZci),(Xs,Cd,YZcj),(Xs,Cd,YZck),(Xs,Cd,YZcl),(Xs,Cd, YZcm),(Xs,Cd,YZcn),(Xs,Cd,YZco),(Xs,Cd,YZcp),(Xs,Cd,YZcq),(Xs,Cd,YZcr),(Xs, Cd,YZcs),(Xs,Cd,YZct),(Xs,Cd,YZcu),(Xs,Cd,YZcv),(Xs,Cd,YZcw),(Xs,Cd,YZcx),(Xs, Cd,YZcy),(Xs,Cd,YZcz),(Xs,Cd,YZda),(Xs,Cd,YZdb),(Xs,Cd,YZdc),(Xs,Cd,YZdd), (Xs,Cd,YZde),(Xs,Cd,YZdf),(Xs,Cd,YZdg),(Xs,Cd,YZdh),(Xs,Cd,YZdi),(Xs,Cd,YZdj), (Xs,Cd,YZdk),(Xs,Cd,YZdl),(Xs,Cd,YZdm),(Xs,Cd,YZdn),(Xs,Cd,YZdo),(Xs,Cd, YZdp),(Xs,Cd,YZdq),(Xs,Cd,YZdr),(Xs,Cd,YZds),(Xs,Cd,YZdt),(Xs,Cd,YZdu),(Xs,Cd, YZdv),(Xs,Cd,YZdw),(Xs,Cd,YZdx),(Xs,Cd,YZdy),(Xs,Cd,YZdz),(Xs,Cd,YZea),(Xs, Cd,YZeb),(Xs,Cd,YZec),(Xs,Cd,YZed),(Xs,Cd,YZee),(Xs,Cd,YZef),(Xs,Cd,YZeg),(Xs, Cd,YZeh),(Xs,Cd,YZei),(Xs,Cd,YZej),(Xs,Cd,YZek),(Xs,Cd,YZel),(Xs,Cd,YZem), (Xs,Cd,YZen),(Xs,Cd,YZeo),(Xs,Cd,YZep),(Xs,Cd,YZeq),(Xs,Cd,YZer),(Xs,Cd,YZes), (Xs,Cd,YZet),(Xs,Cd,YZeu),(Xs,Cd,YZev),(Xs,Cd,YZew),(Xs,Cd,YZex),(Xs,Cd,YZey), (Xs,Cd,YZez),(Xs,Cd,YZfa),(Xs,Cd,YZfb),(Xs,Cd,YZfc),(Xs,Cd,YZfd),(Xs,Cd, YZfe),(Xs,Cd,YZff),(Xs,Cd,YZfg),(Xs,Cd,YZfh),(Xs,Cd,YZfi),(Xs,Cd,YZfj),(Xs,Cd, YZfk),(Xs,Cd,YZfl),(Xs,Cd,YZfm),(Xs,Cd,YZfn),(Xs,Cd,YZfo),(Xs,Cd,YZfp),(Xs,Cd, YZfq),(Xs,Cd,YZfr),(Xs,Cd,YZfs),(Xs,Cd,YZft),(Xs,Cd,YZfu),(Xs,Cd,YZfv),(Xs,Cd, YZfw),(Xs,Cd,YZfx),(Xs,Cd,YZfy),(Xs,Cd,YZfz),(Xs,Cd,YZga),(Xs,Cd,YZgb),(Xs,Cd, YZgc),(Xs,Cd,YZgd),(Xs,Cd,YZge),(Xs,Cd,YZgf),(Xs,Cd,YZgg),(Xs,Cd,YZgh),(Xs, Cd,YZgi),(Xs,Cd,YZgj),(Xs,Cd,YZgk),(Xs,Cd,YZgl),(Xs,Cd,YZgm),(Xs,Cd,YZgn),(Xs, Cd,YZgo),(Xs,Cd,YZgp),(Xs,Cd,YZgq),(Xs,Cd,YZgr),(Xs,Cd,YZgs),(Xs,Cd,YZgt), (Xs,Ce,YZa),(Xs,Ce,YZb),(Xs,Ce,YZc),(Xs,Ce,YZd),(Xs,Ce,YZe),(Xs,Ce,YZf),(Xs, Ce,YZg),(Xs,Ce,YZh),(Xs,Ce,YZi),(Xs,Ce,YZj),(Xs,Ce,YZk),(Xs,Ce,YZl),(Xs,Ce,YZm), (Xs,Ce,YZn),(Xs,Ce,YZo),(Xs,Ce,YZp),(Xs,Ce,YZq),(Xs,Ce,YZr),(Xs,Ce,YZs),(Xs, Ce,YZt),(Xs,Ce,YZu),(Xs,Ce,YZv),(Xs,Ce,YZw),(Xs,Ce,YZx),(Xs,Ce,YZy),(Xs,Ce, YZz),(Xs,Ce,YZaa),(Xs,Ce,YZab),(Xs,Ce,YZac),(Xs,Ce,YZad),(Xs,Ce,YZae),(Xs,Ce, YZaf),(Xs,Ce,YZag),(Xs,Ce,YZah),(Xs,Ce,YZai),(Xs,Ce,YZaj),(Xs,Ce,YZak),(Xs,Ce, YZal),(Xs,Ce,YZam),(Xs,Ce,YZan),(Xs,Ce,YZao),(Xs,Ce,YZap),(Xs,Ce,YZaq),(Xs,Ce, YZar),(Xs,Ce,YZas),(Xs,Ce,YZat),(Xs,Ce,YZau),(Xs,Ce,YZav),(Xs,Ce,YZaw),(Xs, Ce,YZax),(Xs,Ce,YZay),(Xs,Ce,YZaz),(Xs,Ce,YZba),(Xs,Ce,YZbb),(Xs,Ce,YZbc),(Xs, Ce,YZbd),(Xs,Ce,YZbe),(Xs,Ce,YZbf),(Xs,Ce,YZbg),(Xs,Ce,YZbh),(Xs,Ce,YZbi),(Xs, Ce,YZbj),(Xs,Ce,YZbk),(Xs,Ce,YZbl),(Xs,Ce,YZbm),(Xs,Ce,YZbn),(Xs,Ce,YZbo), (Xs,Ce,YZbp),(Xs,Ce,YZbq),(Xs,Ce,YZbr),(Xs,Ce,YZbs),(Xs,Ce,YZbt),(Xs,Ce,YZbu), (Xs,Ce,YZbv),(Xs,Ce,YZbw),(Xs,Ce,YZbx),(Xs,Ce,YZby),(Xs,Ce,YZbz),(Xs,Ce,YZca), (Xs,Ce,YZcb),(Xs,Ce,YZcc),(Xs,Ce,YZcd),(Xs,Ce,YZce),(Xs,Ce,YZcf),(Xs,Ce, YZcg),(Xs,Ce,YZch),(Xs,Ce,YZci),(Xs,Ce,YZcj),(Xs,Ce,YZck),(Xs,Ce,YZcl),(Xs,Ce, YZcm),(Xs,Ce,YZcn),(Xs,Ce,YZco),(Xs,Ce,YZcp),(Xs,Ce,YZcq),(Xs,Ce,YZer),(Xs,Ce, YZcs),(Xs,Ce,YZct),(Xs,Ce,YZcu),(Xs,Ce,YZcv),(Xs,Ce,YZcw),(Xs,Ce,YZcx),(Xs, Ce,YZcy),(Xs,Ce,YZcz),(Xs,Ce,YZda),(Xs,Ce,YZdb),(Xs,Ce,YZdc),(Xs,Ce,YZdd),(Xs, Ce,YZde),(Xs,Ce,YZdf),(Xs,Ce,YZdg),(Xs,Ce,YZdh),(Xs,Ce,YZdi),(Xs,Ce,YZdj),(Xs, Ce,YZdk),(Xs,Ce,YZdl),(Xs,Ce,YZdm),(Xs,Ce,YZdn),(Xs,Ce,YZdo),(Xs,Ce,YZdp), (Xs,Ce,YZdq),(Xs,Cc,YZdr),(Xs,Ce,YZds),(Xs,Ce,YZdt),(Xs,Ce,YZdu),(Xs,Ce,YZdv), (Xs,Ce,YZdw),(Xs,Ce,YZdx),(Xs,Ce,YZdy),(Xs,Ce,YZdz),(Xs,Ce,YZea),(Xs,Ce,YZeb), (Xs,Ce,YZec),(Xs,Ce,YZed),(Xs,Ce,YZee),(Xs,Ce,YZef),(Xs,Ce,YZeg),(Xs,Ce, YZeh),(Xs,Ce,YZei),(Xs,Ce,YZej),(Xs,Ce,YZek),(Xs,Ce,YZel),(Xs,Ce,YZem),(Xs,Ce, YZen),(Xs,Ce,YZeo),(Xs,Ce,YZep),(Xs,Ce,YZeq),(Xs,Ce,YZer),(Xs,Ce,YZes),(Xs,Ce, YZet),(Xs,Ce,YZeu),(Xs,Ce,YZev),(Xs,Ce,YZew),(Xs,Ce,YZex),(Xs,Ce,YZey),(Xs, Ce,YZez),(Xs,Ce,YZfa),(Xs,Ce,YZfb),(Xs,Ce,YZfc),(Xs,Ce,YZfd),(Xs,Ce,YZfe),(Xs, Ce,YZff),(Xs,Ce,YZfg),(Xs,Ce,YZfh),(Xs,Ce,YZfi),(Xs,Ce,YZfj),(Xs,Ce,YZfk),(Xs,Ce, YZfl),(Xs,Ce,YZfm),(Xs,Ce,YZfn),(Xs,Ce,YZfo),(Xs,Ce,YZfp),(Xs,Ce,YZfq),(Xs,Ce, YZfr),(Xs,Ce,YZfs),(Xs,Ce,YZft),(Xs,Ce,YZfu),(Xs,Ce,YZfv),(Xs,Ce,YZfw),(Xs,Ce, YZfx),(Xs,Ce,YZfy),(Xs,Ce,YZfz),(Xs,Ce,YZga),(Xs,Ce,YZgb),(Xs,Ce,YZgc),(Xs,Ce, YZgd),(Xs,Ce,YZge),(Xs,Ce,YZgf),(Xs,Ce,YZgg),(Xs,Ce,YZgh),(Xs,Ce,YZgi),(Xs, Ce,YZgj),(Xs,Ce,YZgk),(Xs,Ce,YZgl),(Xs,Ce,YZgm),(Xs,Ce,YZgn),(Xs,Ce,YZgo),(Xs, Ce,YZgp),(Xs,Ce,YZgq),(Xs,Ce,YZgr),(Xs,Ce,YZgs),(Xs,Ce,YZgt),(Xs,Cf,YZa),(Xs, Cf,YZb),(Xs,Cf,YZc),(Xs,Cf,YZd),(Xs,Cf,YZe),(Xs,Cf,YZf),(Xs,Cf,YZg),(Xs,Cf, YZh),(Xs,Cf,YZi),(Xs,Cf,YZj),(Xs,Cf,YZk),(Xs,Cf,YZl),(Xs,Cf,YZm),(Xs,Cf,YZn),(Xs, Cf,YZo),(Xs,Cf,YZp),(Xs,Cf,YZq),(Xs,Cf,YZr),(Xs,Cf,YZs),(Xs,Cf,YZt),(Xs,Cf,YZu), (Xs,Cf,YZv),(Xs,Cf,YZw),(Xs,Cf,YZx),(Xs,Cf,YZy),(Xs,Cf,YZz),(Xs,Cf,YZaa),(Xs, Cf,YZab),(Xs,Cf,YZac),(Xs,Cf,YZad),(Xs,Cf,YZae),(Xs,Cf,YZaf),(Xs,Cf,YZag),(Xs, Cf,YZah),(Xs,Cf,YZai),(Xs,Cf,YZaj),(Xs,Cf,YZak),(Xs,Cf,YZal),(Xs,Cf,YZam),(Xs, Cf,YZan),(Xs,Cf,YZao),(Xs,Cf,YZap),(Xs,Cf,YZaq),(Xs,Cf,YZar),(Xs,Cf,YZas),(Xs, Cf,YZat),(Xs,Cf,YZau),(Xs,Cf,YZav),(Xs,Cf,YZaw),(Xs,Cf,YZax),(Xs,Cf,YZay),(Xs, Cf,YZaz),(Xs,Cf,YZba),(Xs,Cf,YZbb),(Xs,Cf,YZbc),(Xs,Cf,YZbd),(Xs,Cf,YZbe),(Xs, Cf,YZbf),(Xs,Cf,YZbg),(Xs,Cf,YZbh),(Xs,Cf,YZbi),(Xs,Cf,YZbj),(Xs,Cf,YZbk), (Xs, Cf,YZbl),(Xs,Cf,YZbm),(Xs,Cf,YZbn),(Xs,Cf,YZbo),(Xs,Cf,YZbp),(Xs,Cf,YZbq), (Xs,Cf,YZbr),(Xs,Cf,YZbs),(Xs,Cf,YZbt),(Xs,Cf,YZbu),(Xs,Cf,YZbv),(Xs,Cf,YZbw), (Xs,Cf,YZbx),(Xs,Cf,YZby),(Xs,Cf,YZbz),(Xs,Cf,YZca),(Xs,Cf,YZcb),(Xs,Cf,YZcc), (Xs,Cf,YZcd),(Xs,Cf,YZce),(Xs,Cf,YZcf),(Xs,Cf,YZcg),(Xs,Cf,YZch),(Xs,Cf,YZci),(Xs, Cf,YZcj),(Xs,Cf,YZck),(Xs,Cf,YZcl),(Xs,Cf,YZcm),(Xs,Cf,YZcn),(Xs,Cf,YZco),(Xs, Cf,YZcp),(Xs,Cf,YZcq),(Xs,Cf,YZcr),(Xs,Cf,YZcs),(Xs,Cf,YZct),(Xs,Cf,YZcu),(Xs, Cf,YZcv),(Xs,Cf,YZcw),(Xs,Cf,YZcx),(Xs,Cf,YZcy),(Xs,Cf,YZcz),(Xs,Cf,YZda),(Xs, Cf,YZdb),(Xs,Cf,YZdc),(Xs,Cf,YZdd),(Xs,Cf,YZde),(Xs,Cf,YZdf),(Xs,Cf,YZdg),(Xs, Cf,YZdh),(Xs,Cf,YZdi),(Xs,Cf,YZdj),(Xs,Cf,YZdk),(Xs,Cf,YZdi),(Xs,Cf,YZdm),(Xs, Cf,YZdn),(Xs,Cf,YZdo),(Xs,Cf,YZdp),(Xs,Cf,YZdq),(Xs,Cf,YZdr),(Xs,Cf,YZds),(Xs, Cf,YZdt),(Xs,Cf,YZdu),(Xs,Cf,YZdv),(Xs,Cf,YZdw),(Xs,Cf,YZdx),(Xs,Cf,YZdy),(Xs, Cf,YZdz),(Xs,Cf,YZea),(Xs,Cf,YZeb),(Xs,Cf,YZec),(Xs,Cf,YZed),(Xs,Cf,YZee),(Xs, Cf,YZef),(Xs,CfE,YZeg),(Xs,Cf,YZeh),(Xs,Cf,YZei),(Xs,Cf,YZej),(Xs,Cf,YZek),(Xs, Cf,YZel),(Xs,Cf,YZem),(Xs,Cf,YZen),(Xs,Cf,YZeo),(Xs,Cf,YZep),(Xs,Cf,YZeq),(Xs, Cf,YZer),(Xs,Cf,YZes),(Xs,Cf,YZet),(Xs,Cf,YZeu),(Xs,Cf,YZev),(Xs,Cf,YZew),(Xs, Cf,YZex),(Xs,Cf,YZey),(Xs,Cf,YZez),(Xs,Cf,YZfa),(Xs,Cf,YZfb),(Xs,Cf,YZfc),(Xs, Cf,YZfd),(Xs,Cf,YZfe),(Xs,Cf,YZff),(Xs,Cf,YZfg),(Xs,Cf,YZfh),(Xs,Cf,YZfi),(Xs,Cf, YZfj),(Xs,Cf,YZfk),(Xs,Cf,YZfl),(Xs,Cf,YZfm),(Xs,Cf,YZfn),(Xs,Cf,YZfo),(Xs,Cf,YZfp), (Xs,Cf,YZfq),(Xs,Cf,YZfr),(Xs,Cf,YZfs),(Xs,Cf,YZft),(Xs,Cf,YZfu),(Xs,Cf,YZfv), (Xs,Cf,YZfw),(Xs,Cf,YZfx),(Xs,Cf,YZfy),(Xs,Cf,YZfz),(Xs,Cf,YZga),(Xs,Cf,YZgb), (Xs,Cf,YZgc),(Xs,Cf,YZgd),(Xs,Cf,YZge),(Xs,Cf,YZgf),(Xs,Cf,YZgg),(Xs,Cf,YZgh), (Xs,Cf,YZgi),(Xs,Cf,YZgj),(Xs,Cf,YZgk),(Xs,Cf,YZgl),(Xs,Cf,YZgm),(Xs,Cf,YZgn), (Xs,Cf,YZgo),(Xs,Cf,YZgp),(Xs,Cf,YZgq),(Xs,Cf,YZgr),(Xs,Cf,YZgs),(Xs,Cf,YZgt), (Xt,Ca,YZa),(Xt,Ca,YZb),(Xt,Ca,YZc),(Xt,Ca,YZd),(Xt,Ca,YZe),(Xt,Ca,YZf),(Xt,Ca, YZg),(Xt,Ca,YZh),(Xt,Ca,YZi),(Xt,Ca,YZj),(Xt,Ca,YZk),(Xt,Ca,YZl),(Xt,Ca,YZm),(Xt, Ca,YZn),(Xt,Ca,YZo),(Xt,Ca,YZp),(Xt,Ca,YZq),(Xt,Ca,YZr),(Xt,Ca,YZs),(Xt,Ca, YZt),(Xt,Ca,YZu),(Xt,Ca,YZv),(Xt,Ca,YZw),(Xt,Ca,YZx),(Xt,Ca,YZy),(Xt,Ca,YZz),(Xt, Ca,YZaa),(Xt,Ca,YZab),(Xt,Ca,YZac),(Xt,Ca,YZad),(Xt,Ca,YZae),(Xt,Ca,YZaf),(Xt, Ca,YZag),(Xt,Ca,YZah),(Xt,Ca,YZai),(Xt,Ca,YZaj),(Xt,Ca,YZak),(Xt,Ca,YZal),(Xt, Ca,YZam),(Xt,Ca,YZan),(Xt,Ca,YZao),(Xt,Ca,YZap),(Xt,Ca,YZaq),(Xt,Ca,YZar),(Xt, Ca,YZas),(Xt,Ca,YZat),(Xt,Ca,YZau),(Xt,Ca,YZav),(Xt,Ca,YZaw),(Xt,Ca,YZax),(Xt, Ca,YZay),(Xt,Ca,YZaz),(Xt,Ca,YZba),(Xt,Ca,YZbb),(Xt,Ca,YZbc),(Xt,Ca,YZbd),(Xt, Ca,YZbe),(Xt,Ca,YZbf),(Xt,Ca,YZbg),(Xt,Ca,YZbh),(Xt,Ca,YZbi),(Xt,Ca,YZbj),(Xt, Ca,YZbk),(Xt,Ca,YZbl),(Xt,Ca,YZbm),(Xt,Ca,YZbn),(Xt,Ca,YZbo),(Xt,Ca,YZbp),(Xt, Ca,YZbq),(Xt,Ca,YZbr),(Xt,Ca,YZbs),(Xt,Ca,YZbt),(Xt,Ca,YZbu),(Xt,Ca,YZbv),(Xt, Ca,YZbw),(Xt,Ca,YZbx),(Xt,Ca,YZby),(Xt,Ca,YZbz),(Xt,Ca,YZca),(Xt,Ca,YZcb),(Xt, Ca,YZcc),(Xt,Ca,YZcd),(Xt,Ca,YZce),(Xt,Ca,YZcf),(Xt,Ca,YZcg),(Xt,Ca,YZch),(Xt, Ca,YZci),(Xt,Ca,YZcj),(Xt,Ca,YZck),(Xt,Ca,YZcl),(Xt,Ca,YZcm),(Xt,Ca,YZcn),(Xt, Ca,YZco),(Xt,Ca,YZcp),(Xt,Ca,YZcq),(Xt,Ca,YZcr),(Xt,Ca,YZcs),(Xt,Ca,YZct),(Xt,Ca, YZcu),(Xt,Ca,YZcv),(Xt,Ca,YZcw),(Xt,Ca,YZcx),(Xt,Ca,YZcy),(Xt,Ca,YZcz),(Xt,Ca, YZda),(Xt,Ca,YZdb),(Xt,Ca,YZdc),(Xt,Ca,YZdd),(Xt,Ca,YZde),(Xt,Ca,YZdf),(Xt,Ca, YZdg),(Xt,Ca,YZdh),(Xt,Ca,YZdi),(Xt,Ca,YZdj),(Xt,Ca,YZdk),(Xt,Ca,YZdl),(Xt,Ca, YZdm),(Xt,Ca,YZdn),(Xt,Ca,YZdo),(Xt,Ca,YZdp),(Xt,Ca,YZdq),(Xt,Ca,YZdr),(Xt,Ca, YZds),(Xt,Ca,YZdt),(Xt,Ca,YZdu),(Xt,Ca,YZdv),(Xt,Ca,YZdw),(Xt,Ca,YZdx),(Xt,Ca, YZdy),(Xt,Ca,YZdz),(Xt,Ca,YZea),(Xt,Ca,YZeb),(Xt,Ca,YZec),(Xt,Ca,YZed),(Xt,Ca, YZee),(Xt,Ca,YZef),(Xt,Ca,YZeg),(Xt,Ca,YZeh),(Xt,Ca,YZei),(Xt,Ca,YZej),(Xt,Ca, YZek),(Xt,Ca,YZel),(Xt,Ca,YZem),(Xt,Ca,YZen),(Xt,Ca,YZeo),(Xt,Ca,YZep),(Xt,Ca, YZeq),(Xt,Ca,YZer),(Xt,Ca,YZes),(Xt,Ca,YZet),(Xt,Ca,YZeu),(Xt,Ca,YZev),(Xt,Ca, YZew),(Xt,Ca,YZex),(Xt,Ca,YZey),(Xt,Ca,YZez),(Xt,Ca,YZfa),(Xt,Ca,YZfb),(Xt,Ca, YZfc),(Xt,Ca,YZfd),(Xt,Ca,YZfe),(Xt,Ca,YZff),(Xt,Ca,YZfg),(Xt,Ca,YZfh),(Xt,Ca,YZfi), (Xt,Ca,YZfj),(Xt,Ca,YZfk),(Xt,Ca,YZfl),(Xt,Ca,YZfm),(Xt,Ca,YZfn),(Xt,Ca,YZfo), (Xt,Ca,YZfp),(Xt,Ca,YZfq),(Xt,Ca,YZfr),(Xt,Ca,YZfs),(Xt,Ca,YZft),(Xt,Ca,YZfu), (Xt,Ca,YZfv),(Xt,Ca,YZfw),(Xt,Ca,YZfx),(Xt,Ca,YZfy),(Xt,Ca,YZfz),(Xt,Ca,YZga),(Xt, Ca,YZgb),(Xt,Ca,YZgc),(Xt,Ca,YZgd),(Xt,Ca,YZge),(Xt,Ca,YZgf),(Xt,Ca,YZgg),(Xt, Ca,YZgh),(Xt,Ca,YZgi),(Xt,Ca,YZgj),(Xt,Ca,YZgk),(Xt,Ca,YZgl),(Xt,Ca,YZgm),(Xt, Ca,YZgn),(Xt,Ca,YZgo),(Xt,Ca,YZgp),(Xt,Ca,YZgq),(Xt,Ca,YZgr),(Xt,Ca,YZgs),(Xt, Ca,YZgt),(Xt,Cb,YZa),(Xt,Cb,YZb),(Xt,Cb,YZc),(Xt,Cb,YZd),(Xt,Cb,YZe),(Xt,Cb, YZf),(Xt,Cb,YZg),(Xt,Cb,YZh),(Xt,Cb,YZi),(Xt,Cb,YZj),(Xt,Cb,YZk),(Xt,Cb,YZl),(Xt, Cb,YZm),(Xt,Cb,YZn),(Xt,Cb,YZo),(Xt,Cb,YZp),(Xt,Cb,YZq),(Xt,Cb,YZr),(Xt,Cb, YZs),(Xt,Cb,YZt),(Xt,Cb,YZu),(Xt,Cb,YZv),(Xt,Cb,YZw),(Xt,Cb,YZx),(Xt,Cb,YZy), (Xt,Cb,YZz),(Xt,Cb,YZaa),(Xt,Cb,YZab),(Xt,Cb,YZac),(Xt,Cb,YZad),(Xt,Cb,YZae), (Xt,Cb,YZaf),(Xt,Cb,YZag),(Xt,Cb,YZah),(Xt,Cb,YZai),(Xt,Cb,YZaj),(Xt,Cb,YZak), (Xt,Cb,YZal),(Xt,Cb,YZam),(Xt,Cb,YZan),(Xt,Cb,YZao),(Xt,Cb,YZap),(Xt,Cb,YZaq), (Xt,Cb,YZar),(Xt,Cb,YZas),(Xt,Cb,YZat),(Xt,Cb,YZau),(Xt,Cb,YZav),(Xt,Cb,YZaw), (Xt,Cb,YZax),(Xt,Cb,YZay),(Xt,Cb,YZaz),(Xt,Cb,YZba),(Xt,Cb,YZbb),(Xt,Cb,YZbc), (Xt,Cb,YZbd),(Xt,Cb,YZbe),(Xt,Cb,YZbf),(Xt,Cb,YZbg),(Xt,Cb,YZbh),(Xt,Cb,YZbi), (Xt,Cb,YZbj),(Xt,Cb,YZbk),(Xt,Cb,YZbl),(Xt,Cb,YZbm),(Xt,Cb,YZbn),(Xt,Cb,YZbo), (Xt,Cb,YZbp),(Xt,Cb,YZbq),(Xt,Cb,YZbr),(Xt,Cb,YZbs),(Xt,Cb,YZbt),(Xt,Cb,YZbu), (Xt,Cb,YZbv),(Xt,Cb,YZbw),(Xt,Cb,YZbx),(Xt,Cb,YZby),(Xt,Cb,YZbz),(Xt,Cb, YZca),(Xt,Cb,YZcb),(Xt,Cb,YZcc),(Xt,Cb,YZcd),(Xt,Cb,YZce),(Xt,Cb,YZcf),(Xt,Cb, YZcg),(Xt,Cb,YZch),(Xt,Cb,YZci),(Xt,Cb,YZcj),(Xt,Cb,YZck),(Xt,Cb,YZcl),(Xt,Cb, YZcm),(Xt,Cb,YZcn),(Xt,Cb,YZco),(Xt,Cb,YZcp),(Xt,Cb,YZcq),(Xt,Cb,YZcr),(Xt,Cb, YZcs),(Xt,Cb,YZct),(Xt,Cb,YZcu),(Xt,Cb,YZcv),(Xt,Cb,YZcw),(Xt,Cb,YZcx),(Xt, Cb,YZcy),(Xt,Cb,YZcz),(Xt,Cb,YZda),(Xt,Cb,YZdb),(Xt,Cb,YZdc),(Xt,Cb,YZdd),(Xt, Cb,YZde),(Xt,Cb,YZdf),(Xt,Cb,YZdg),(Xt,Cb,YZdh),(Xt,Cb,YZdi),(Xt,Cb,YZdj),(Xt, Cb,YZdk),(Xt,Cb,YZdl),(Xt,Cb,YZdm),(Xt,Cb,YZdn),(Xt,Cb,YZdo),(Xt,Cb,YZdp),(Xt, Cb,YZdq),(Xt,Cb,YZdr),(Xt,Cb,YZds),(Xt,Cb,YZdt),(Xt,Cb,YZdu),(Xt,Cb,YZdv),(Xt, Cb,YZdw),(Xt,Cb,YZdx),(Xt,Cb,YZdy),(Xt,Cb,YZdz),(Xt,Cb,YZea),(Xt,Cb,YZeb), (Xt,Cb,YZec),(Xt,Cb,YZed),(Xt,Cb,YZee),(Xt,Cb,YZef),(Xt,Cb,YZeg),(Xt,Cb,YZeh), (Xt,Cb,YZei),(Xt,Cb,YZej),(Xt,Cb,YZek),(Xt,Cb,YZel),(Xt,Cb,YZem),(Xt,Cb,YZen), (Xt,Cb,YZeo),(Xt,Cb,YZep),(Xt,Cb,YZeq),(Xt,Cb,YZer),(Xt,Cb,YZes),(Xt,Cb,YZet), (Xt,Cb,YZeu),(Xt,Cb,YZev),(Xt,Cb,YZew),(Xt,Cb,YZex),(Xt,Cb,YZey),(Xt,Cb,YZez), (Xt,Cb,YZfa),(Xt,Cb,YZfb),(Xt,Cb,YZfc),(Xt,Cb,YZfd),(Xt,Cb,YZfe),(Xt,Cb,YZff), (Xt,Cb,YZfg),(Xt,Cb,YZfh),(Xt,Cb,YZfi),(Xt,Cb,YZfj),(Xt,Cb,YZfk),(Xt,Cb,YZfl),(Xt, Cb,YZfm),(Xt,Cb,YZfn),(Xt,Cb,YZfo),(Xt,Cb,YZfp),(Xt,Cb,YZfq),(Xt,Cb,YZfr),(Xt, Cb,YZfs),(Xt,Cb,YZft),(Xt,Cb,YZfu),(Xt,Cb,YZfv),(Xt,Cb,YZfw),(Xt,Cb,YZfx),(Xt, Cb,YZfy),(Xt,Cb,YZfz),(Xt,Cb,YZga),(Xt,Cb,YZgb),(Xt,Cb,YZgc),(Xt,Cb,YZgd),(Xt, Cb,YZge),(Xt,Cb,YZgf),(Xt,Cb,YZgg),(Xt,Cb,YZgh),(Xt,Cb,YZgi),(Xt,Cb,YZgj),(Xt, Cb,YZgk),(Xt,Cb,YZgl),(Xt,Cb,YZgm),(Xt,Cb,YZgn),(Xt,Cb,YZgo),(Xt,Cb,YZgp),(Xt, Cb,YZgq),(Xt,Cb,YZgr),(Xt,Cb,YZgs),(Xt,Cb,YZgt),(Xt,Cc,YZa),(Xt,Cc,YZb),(Xt, Cc,YZc),(Xt,Cc,YZd),(Xt,Cc,YZe),(Xt,Cc,YZf),(Xt,Cc,YZg),(Xt,Cc,YZh),(Xt,Cc,YZi), (Xt,Cc,YZj),(Xt,Cc,YZk),(Xt,Cc,YZl),(Xt,Cc,YZm),(Xt,Cc,YZn),(Xt,Cc,YZo),(Xt, Cc,YZp),(Xt,Cc,YZq),(Xt,Cc,YZr),(Xt,Cc,YZs),(Xt,Cc,YZt),(Xt,Cc,YZu),(Xt,Cc,YZv), (Xt,Cc,YZw),(Xt,Cc,YZx),(Xt,Cc,YZy),(Xt,Cc,YZz),(Xt,Cc,YZaa),(Xt,Cc,YZab),(Xt, Cc,YZac),(Xt,Cc,YZad),(Xt,Cc,YZae),(Xt,Cc,YZaf),(Xt,Cc,YZag),(Xt,Cc,YZah),(Xt, Cc,YZai),(Xt,Cc,YZaj),(Xt,Cc,YZak),(Xt,Cc,YZal),(Xt,Cc,YZam),(Xt,Cc,YZan),(Xt, Cc,YZao),(Xt,Cc,YZap),(Xt,Cc,YZaq),(Xt,Cc,YZar),(Xt,Cc,YZas),(Xt,Cc,YZat),(Xt,Cc, YZau),(Xt,Cc,YZav),(Xt,Cc,YZaw),(Xt,Cc,YZax),(Xt,Cc,YZay),(Xt,Cc,YZaz),(Xt,Cc, YZba),(Xt,Cc,YZbb),(Xt,Cc,YZbc),(Xt,Cc,YZbd),(Xt,Cc,YZbe),(Xt,Cc,YZbf),(Xt,Cc, YZbg),(Xt,Cc,YZbh),(Xt,Cc,YZbi),(Xt,Cc,YZbj),(Xt,Cc,YZbk),(Xt,Cc,YZbl),(Xt,Cc, YZbm),(Xt,Cc,YZbn),(Xt,Cc,YZbo),(Xt,Cc,YZbp),(Xt,Cc,YZbq),(Xt,Cc,YZbr),(Xt,Cc, YZbs),(Xt,Cc,YZbt),(Xt,Cc,YZbu),(Xt,Cc,YZbv),(Xt,Cc,YZbw),(Xt,Cc,YZbx),(Xt, Cc,YZby),(Xt,Cc,YZbz),(Xt,Cc,YZca),(Xt,Cc,YZcb),(Xt,Cc,YZcc),(Xt,Cc,YZcd),(Xt, Cc,YZce),(Xt,Cc,YZcf),(Xt,Cc,YZcg),(Xt,Cc,YZch),(Xt,Cc,YZci),(Xt,Cc,YZcj),(Xt,Cc, YZck),(Xt,Cc,YZcl),(Xt,Cc,YZcm),(Xt,Cc,YZcn),(Xt,Cc,YZco),(Xt,Cc,YZcp),(Xt,Cc, YZcq),(Xt,Cc,YZcr),(Xt,Cc,YZcs),(Xt,Cc,YZct),(Xt,Cc,YZcu),(Xt,Cc,YZcv),(Xt,Cc, YZcw),(Xt,Cc,YZcx),(Xt,Cc,YZcy),(Xt,Cc,YZcz),(Xt,Cc,YZda),(Xt,Cc,YZdb),(Xt,Cc, YZdc),(Xt,Cc,YZdd),(Xt,Cc,YZde),(Xt,Cc,YZdf),(Xt,Cc,YZdg),(Xt,Cc,YZdh),(Xt,Cc, YZdi),(Xt,Cc,YZdj),(Xt,Cc,YZdk),(Xt,Cc,YZdl),(Xt,Cc,YZdm),(Xt,Cc,YZdn),(Xt,Cc, YZdo),(Xt,Cc,YZdp),(Xt,Cc,YZdq),(Xt,Cc,YZdr),(Xt,Cc,YZds),(Xt,Cc,YZdt),(Xt,Cc, YZdu),(Xt,Cc,YZdv),(Xt,Cc,YZdw),(Xt,Cc,YZdx),(Xt,Cc,YZdy),(Xt,Cc,YZdz),(Xt,Cc, YZea),(Xt,Cc,YZeb),(Xt,Cc,YZec),(Xt,Cc,YZed),(Xt,Cc,YZee),(Xt,Cc,YZef),(Xt,Cc, YZeg),(Xt,Cc,YZeh),(Xt,Cc,YZei),(Xt,Cc,YZej),(Xt,Cc,YZek),(Xt,Cc,YZel),(Xt,Cc, YZem),(Xt,Cc,YZen),(Xt,Cc,YZeo),(Xt,Cc,YZep),(Xt,Cc,YZeq),(Xt,Cc,YZer),(Xt,Cc, YZes),(Xt,Cc,YZet),(Xt,Cc,YZeu),(Xt,Cc,YZev),(Xt,Cc,YZew),(Xt,Cc,YZex),(Xt,Cc, YZey),(Xt,Cc,YZez),(Xt,Cc,YZfa),(Xt,Cc,YZfb),(Xt,Cc,YZfc),(Xt,Cc,YZfd),(Xt,Cc,YZfe), (Xt,Cc,YZff),(Xt,Cc,YZfg),(Xt,Cc,YZfh),(Xt,Cc,YZfi),(Xt,Cc,YZfj),(Xt,Cc,YZfk), (Xt,Cc,YZfl),(Xt,Cc,YZfm),(Xt,Cc,YZfn),(Xt,Cc,YZfo),(Xt,Cc,YZfp),(Xt,Cc,YZfq), (Xt,Cc,YZfr),(Xt,Cc,YZfs),(Xt,Cc,YZft),(Xt,Cc,YZfu),(Xt,Cc,YZfv),(Xt,Cc,YZfw),(Xt, Cc,YZfx),(Xt,Cc,YZfy),(Xt,Cc,YZfz),(Xt,Cc,YZga),(Xt,Cc,YZgb),(Xt,Cc,YZgc),(Xt, Cc,YZgd),(Xt,Cc,YZge),(Xt,Cc,YZgf),(Xt,Cc,YZgg),(Xt,Cc,YZgh),(Xt,Cc,YZgi),(Xt, Cc,YZgj),(Xt,Cc,YZgk),(Xt,Cc,YZgl),(Xt,Cc,YZgm),(Xt,Cc,YZgn),(Xt,Cc,YZgo),(Xt, Cc,YZgp),(Xt,Cc,YZgq),(Xt,Cc,YZgr),(Xt,Cc,YZgs),(Xt,Cc,YZgt),(Xt,Cd,YZa),(Xt,Cd, YZb),(Xt,Cd,YZc),(Xt,Cd,YZd),(Xt,Cd,YZe),(Xt,Cd,YZf),(Xt,Cd,YZg),(Xt,Cd,YZh), (Xt,Cd,YZi),(Xt,Cd,YZj),(Xt,Cd,YZk),(Xt,Cd,YZl),(Xt,Cd,YZm),(Xt,Cd,YZn),(Xt,Cd, YZo),(Xt,Cd,YZp),(Xt,Cd,YZq),(Xt,Cd,YZr),(Xt,Cd,YZs),(Xt,Cd,YZt),(Xt,Cd,YZu), (Xt,Cd,YZv),(Xt,Cd,YZw),(Xt,Cd,YZx),(Xt,Cd,YZy),(Xt,Cd,YZz),(Xt,Cd,YZaa),(Xt, Cd,YZab),(Xt,Cd,YZac),(Xt,Cd,YZad),(Xt,Cd,YZae),(Xt,Cd,YZaf),(Xt,Cd,YZag),(Xt, Cd,YZah),(Xt,Cd,YZai),(Xt,Cd,YZaj),(Xt,Cd,YZak),(Xt,Cd,YZal),(Xt,Cd,YZam),(Xt, Cd,YZan),(Xt,Cd,YZao),(Xt,Cd,YZap),(Xt,Cd,YZaq),(Xt,Cd,YZar),(Xt,Cd,YZas),(Xt, Cd,YZat),(Xt,Cd,YZau),(Xt,Cd,YZav),(Xt,Cd,YZaw),(Xt,Cd,YZax),(Xt,Cd,YZay),(Xt, Cd,YZaz),(Xt,Cd,YZba),(Xt,Cd,YZbb),(Xt,Cd,YZbc),(Xt,Cd,YZbd),(Xt,Cd,YZbe),(Xt, Cd,YZbf),(Xt,Cd,YZbg),(Xt,Cd,YZbh),(Xt,Cd,YZbi),(Xt,Cd,YZbj),(Xt,Cd,YZbk),(Xt, Cd,YZbl),(Xt,Cd,YZbm),(Xt,Cd,YZbn),(Xt,Cd,YZbo),(Xt,Cd,YZbp),(Xt,Cd,YZbq),(Xt, Cd,YZbr),(Xt,Cd,YZbs),(Xt,Cd,YZbt),(Xt,Cd,YZbu),(Xt,Cd,YZbv),(Xt,Cd,YZbw), (Xt,Cd,YZbx),(Xt,Cd,YZby),(Xt,Cd,YZbz),(Xt,Cd,YZca),(Xt,Cd,YZcb),(Xt,Cd,YZcc), (Xt,Cd,YZcd),(Xt,Cd,YZce),(Xt,Cd,YZcf),(Xt,Cd,YZcg),(Xt,Cd,YZch),(Xt,Cd,YZci), (Xt,Cd,YZcj),(Xt,Cd,YZck),(Xt,Cd,YZcl),(Xt,Cd,YZcm),(Xt,Cd,YZcn),(Xt,Cd,YZco), (Xt,Cd,YZcp),(Xt,Cd,YZcq),(Xt,Cd,YZcr),(Xt,Cd,YZcs),(Xt,Cd,YZct),(Xt,Cd,YZcu),(Xt, Cd,YZcv),(Xt,Cd,YZcw),(Xt,Cd,YZcx),(Xt,Cd,YZcy),(Xt,Cd,YZcz),(Xt,Cd,YZda), (Xt,Cd,YZdb),(Xt,Cd,YZdc),(Xt,Cd,YZdd),(Xt,Cd,YZde),(Xt,Cd,YZdf),(Xt,Cd,YZdg), (Xt,Cd,YZdh),(Xt,Cd,YZdi),(Xt,Cd,YZdj),(Xt,Cd,YZdk),(Xt,Cd,YZdl),(Xt,Cd,YZdm), (Xt,Cd,YZdn),(Xt,Cd,YZdo),(Xt,Cd,YZdp),(Xt,Cd,YZdq),(Xt,Cd,YZdr),(Xt,Cd,YZds), (Xt,Cd,YZdt),(Xt,Cd,YZdu),(Xt,Cd,YZdv),(Xt,Cd,YZdw),(Xt,Cd,YZdx),(Xt,Cd,YZdy), (Xt,Cd,YZdz),(Xt,Cd,YZea),(Xt,Cd,YZeb),(Xt,Cd,YZec),(Xt,Cd,YZed),(Xt,Cd,YZee), (Xt,Cd,YZef),(Xt,Cd,YZeg),(Xt,Cd,YZeh),(Xt,Cd,YZei),(Xt,Cd,YZej),(Xt,Cd,YZek), (Xt,Cd,YZel),(Xt,Cd,YZem),(Xt,Cd,YZen),(Xt,Cd,YZeo),(Xt,Cd,YZep),(Xt,Cd, YZeq),(Xt,Cd,YZer),(Xt,Cd,YZes),(Xt,Cd,YZet),(Xt,Cd,YZeu),(Xt,Cd,YZev),(Xt,Cd, YZew),(Xt,Cd,YZex),(Xt,Cd,YZey),(Xt,Cd,YZez),(Xt,Cd,YZfa),(Xt,Cd,YZfb),(Xt,Cd, YZfc),(Xt,Cd,YZfd),(Xt,Cd,YZfe),(Xt,Cd,YZff),(Xt,Cd,YZfg),(Xt,Cd,YZfh),(Xt,Cd, YZfi),(Xt,Cd,YZfj),(Xt,Cd,YZfk),(Xt,Cd,YZfl),(Xt,Cd,YZfm),(Xt,Cd,YZfn),(Xt,Cd,YZfo), (Xt,Cd,YZfp),(Xt,Cd,YZfq),(Xt,Cd,YZfr),(Xt,Cd,YZfs),(Xt,Cd,YZft),(Xt,Cd,YZfu), (Xt,Cd,YZfv),(Xt,Cd,YZfw),(Xt,Cd,YZfx),(Xt,Cd,YZfy),(Xt,Cd,YZfz),(Xt,Cd,YZga), (Xt,Cd,YZgb),(Xt,Cd,YZgc),(Xt,Cd,YZgd),(Xt,Cd,YZge),(Xt,Cd,YZgf),(Xt,Cd,YZgg), (Xt,Cd,YZgh),(Xt,Cd,YZgi),(Xt,Cd,YZgj),(Xt,Cd,YZgk),(Xt,Cd,YZgl),(Xt,Cd,YZgm), (Xt,Cd,YZgn),(Xt,Cd,YZgo),(Xt,Cd,YZgp),(Xt,Cd,YZgq),(Xt,Cd,YZgr),(Xt,Cd, YZgs),(Xt,Cd,YZgt),(Xt,Ce,YZa),(Xt,Ce,YZb),(Xt,Ce,YZc),(Xt,Ce,YZd),(Xt,Ce,YZe), (Xt,Ce,YZf),(Xt,Ce,YZg),(Xt,Ce,YZh),(Xt,Ce,YZi),(Xt,Ce,YZj),(Xt,Ce,YZk),(Xt,Ce, YZl),(Xt,Ce,YZm),(Xt,Ce,YZn),(Xt,Ce,YZo),(Xt,Ce,YZp),(Xt,Ce,YZq),(Xt,Ce,YZr),(Xt, Ce,YZs),(Xt,Ce,YZt),(Xt,Ce,YZu),(Xt,Ce,YZv),(Xt,Ce,YZw),(Xt,Ce,YZx),(Xt,Ce, YZy),(Xt,Ce,YZz),(Xt,Ce,YZaa),(Xt,Ce,YZab),(Xt,Ce,YZac),(Xt,Ce,YZad),(Xt,Ce,YZae), (Xt,Ce,YZaf),(Xt,Ce,YZag),(Xt,Ce,YZah),(Xt,Ce,YZai),(Xt,Ce,YZaj),(Xt,Ce,YZak), (Xt,Ce,YZal),(Xt,Ce,YZam),(Xt,Ce,YZan),(Xt,Ce,YZao),(Xt,Ce,YZap),(Xt,Ce,YZaq), (Xt,Ce,YZar),(Xt,Ce,YZas),(Xt,Ce,YZat),(Xt,Ce,YZau),(Xt,Ce,YZav),(Xt,Ce,YZaw), (Xt,Ce,YZax),(Xt,Ce,YZay),(Xt,Ce,YZaz),(Xt,Ce,YZba),(Xt,Ce,YZbb),(Xt,Ce,YZbc), (Xt,Ce,YZbd),(Xt,Ce,YZbe),(Xt,Ce,YZbf),(Xt,Ce,YZbg),(Xt,Ce,YZbh),(Xt,Ce,YZbi), (Xt,Ce,YZbj),(Xt,Ce,YZbk),(Xt,Ce,YZbl),(Xt,Ce,YZbm),(Xt,Ce,YZbn),(Xt,Ce,YZbo), (Xt,Ce,YZbp),(Xt,Ce,YZbq),(Xt,Ce,YZbr),(Xt,Ce,YZbs),(Xt,Ce,YZbt),(Xt,Ce,YZbu), (Xt,Ce,YZbv),(Xt,Ce,YZbw),(Xt,Ce,YZbx),(Xt,Ce,YZby),(Xt,Ce,YZbz),(Xt,Ce,YZca), (Xt,Ce,YZcb),(Xt,Ce,YZcc),(Xt,Ce,YZcd),(Xt,Ce,YZce),(Xt,Ce,YZcf),(Xt,Ce,YZcg), (Xt,Ce,YZch),(Xt,Ce,YZci),(Xt,Ce,YZcj),(Xt,Ce,YZck),(Xt,Ce,YZel),(Xt,Ce,YZcm), (Xt,Ce,YZcn),(Xt,Ce,YZco),(Xt,Ce,YZcp),(Xt,Ce,YZcq),(Xt,Ce,YZcr),(Xt,Ce,YZcs), (Xt,Ce,YZct),(Xt,Ce,YZcu),(Xt,Ce,YZcv),(Xt,Ce,YZew),(Xt,Ce,YZcx),(Xt,Ce,YZcy), (Xt,Ce,YZcz),(Xt,Ce,YZda),(Xt,Ce,YZdb),(Xt,Ce,YZdc),(Xt,Ce,YZdd),(Xt,Ce,YZde), (Xt,Ce,YZdf),(Xt,Ce,YZdg),(Xt,Ce,YZdh),(Xt,Ce,YZdi),(Xt,Ce,YZdj),(Xt,Ce,YZdk), (Xt,Ce,YZdl),(Xt,Ce,YZdm),(Xt,Ce,YZdn),(Xt,Ce,YZdo),(Xt,Ce,YZdp),(Xt,Ce,YZdq), (Xt,Ce,YZdr),(Xt,Ce,YZds),(Xt,Ce,YZdt),(Xt,Ce,YZdu),(Xt,Ce,YZdv),(Xt,Ce,YZdw), (Xt,Ce,YZdx),(Xt,Ce,YZdy),(Xt,Ce,YZdz),(Xt,Ce,YZea),(Xt,Ce,YZeb),(Xt,Ce,YZec), (Xt,Ce,YZed),(Xt,Ce,YZee),(Xt,Ce,YZef),(Xt,Ce,YZeg),(Xt,Ce,YZeh),(Xt,Ce,YZej), (Xt,Ce,YZej),(Xt,Ce,YZek),(Xt,Ce,YZel),(Xt,Ce,YZem),(Xt,Ce,YZen),(Xt,Ce,YZeo), (Xt,Ce,YZep),(Xt,Ce,YZeq),(Xt,Ce,YZer),(Xt,Ce,YZes),(Xt,Ce,YZet),(Xt,Ce,YZeu), (Xt,Ce,YZev),(Xt,Ce,YZew),(Xt,Ce,YZex),(Xt,Ce,YZey),(Xt,Ce,YZez),(Xt,Ce,YZfa), (Xt,Ce,YZfb),(Xt,Ce,YZfc),(Xt,Ce,YZfd),(Xt,Ce,YZfe),(Xt,Ce,YZff),(Xt,Ce,YZfg),(Xt, Ce,YZfh),(Xt,Ce,YZfi),(Xt,Ce,YZfj),(Xt,Ce,YZfk),(Xt,Ce,YZfl),(Xt,Ce,YZfm),(Xt,Ce, YZfn),(Xt,Ce,YZfo),(Xt,Ce,YZfp),(Xt,Ce,YZfq),(Xt,Ce,YZfr),(Xt,Ce,YZfs),(Xt,Ce, YZft),(Xt,Ce,YZfu),(Xt,Ce,YZfv),(Xt,Ce,YZfw),(Xt,Ce,YZfx),(Xt,Ce,YZfy),(Xt,Ce,YZfz), (Xt,Ce,YZga),(Xt,Ce,YZgb),(Xt,Ce,YZgc),(Xt,Ce,YZgd),(Xt,Ce,YZge),(Xt,Ce,YZgf), (Xt,Ce,YZgg),(Xt,Ce,YZgh),(Xt,Ce,YZgi),(Xt,Ce,YZgj),(Xt,Ce,YZgk),(Xt,Ce,YZgl), (Xt,Ce,YZgm),(Xt,Ce,YZgn),(Xt,Ce,YZgo),(Xt,Ce,YZgp),(Xt,Ce,YZgq),(Xt,Ce, YZgr),(Xt,Ce,YZgs),(Xt,Ce,YZgt),(Xt,Cf,YZa),(Xt,Cf,YZb),(Xt,Cf,YZc),(Xt,Cf,YZd), (Xt,Cf,YZe),(Xt,Cf,YZf),(Xt,Cf,YZg),(Xt,Cf,YZh),(Xt,Cf,xYZi),(Xt,Cf,YZj),(Xt,Cf,YZk), (Xt,Cf,YZl),(Xt,Cf,YZm),(Xt,Cf,YZn),(Xt,Cf,YZo),(Xt,Cf,YZp),(Xt,Cf,YZq),(Xt, Cf,YZr),(Xt,Cf,YZs),(Xt,Cf,YZt),(Xt,Cf,YZu),(Xt,Cf,YZv),(Xt,Cf,YZw),(Xt,Cf,YZx), (Xt,Cf,YZy),(Xt,Cf,YZz),(Xt,Cf,YZaa),(Xt,Cf,YZab),(Xt,Cf,YZac),(Xt,Cf,YZad),(Xt, Cf,YZae),(Xt,Cf,YZaf),(Xt,Cf,YZag),(Xt,Cf,YZah),(Xt,Cf,YZai),(Xt,Cf,YZaj),(Xt,Cf, YZak),(Xt,Cf,YZal),(Xt,Cf,YZam),(Xt,Cf,YZan),(Xt,Cf,YZao),(Xt,Cf,YZap),(Xt,Cf,YZaq), (Xt,Cf,YZar),(Xt,Cf,YZas),(Xt,Cf,YZat),(Xt,Cf,YZau),(Xt,Cf,YZav),(Xt,Cf,YZaw), (Xt,Cf,YZax),(Xt,Cf,YZay),(Xt,Cf,YZaz),(Xt,Cf,YZba),(Xt,Cf,YZbb),(Xt,Cf,YZbc), (Xt,Cf,YZbd),(Xt,Cf,YZbe),(Xt,Cf,YZbf),(Xt,Cf,YZbg),(Xt,Cf,YZbh),(Xt,Cf,YZbi),(Xt, Cf,YZbj),(Xt,Cf,YZbk),(Xt,Cf,YZbl),(Xt,Cf,YZbm),(Xt,Cf,YZbn),(Xt,Cf,YZbo),(Xt, Cf,YZbp),(Xt,Cf,YZbq),(Xt,Cf,YZbr),(Xt,Cf,YZbs),(Xt,Cf,YZbt),(Xt,Cf,YZbu),(Xt, Cf,YZbv),(Xt,Cf,YZbw),(Xt,Cf,YZbx),(Xt,Cf,YZby),(Xt,Cf,YZbz),(Xt,Cf,YZca),(Xt, Cf,YZcb),(Xt,Cf,YZcc),(Xt,Cf,YZcd),(Xt,Cf,YZce),(Xt,Cf,YZcf),(Xt,Cf,YZcg),(Xt,Cf, YZch),(Xt,Cf,YZci),(Xt,Cf,YZcj),(Xt,Cf,YZck),(Xt,Cf,YZcl),(Xt,Cf,YZem),(Xt,Cf,YZcn), (Xt,Cf,YZco),(Xt,Cf,YZcp),(Xt,Cf,YZcq),(Xt,Cf,YZcr),(Xt,Cf,YZcs),(Xt,Cf,YZct), (Xt,Cf,YZCu),(Xt,Cf,YZCv),(Xt,Cf,YZCw),(Xt,Cf,YZCx),(Xt,Cf,YZcy),(Xt,Cf,YZez), (Xt,Cf,YZda),(Xt,Cf,YZdb),(Xt,Cf,YZdc),(Xt,Cf,YZdd),(Xt,Cf,YZde),(Xt,Cf,YZdf),(Xt, Cf,YZdg),(Xt,Cf,YZdh),(Xt,Cf,YZdi),(Xt,Cf,YZdj),(Xt,Cf,YZdk),(Xt,Cf,YZdl),(Xt, Cf,YZdm),(Xt,Cf,YZdn),(Xt,Cf,YZdo),(Xt,Cf,YZdp),(Xt,Cf,YZdq),(Xt,Cf,YZdr),(Xt, Cf,YZds),(Xt,Cf,YZdt),(Xt,Cf,YZdu),(Xt,Cf,YZdv),(Xt,Cf,YZdw),(Xt,Cf,YZdx),(Xt,Cf, YZdy),(Xt,Cf,YZdz),(Xt,Cf,YZea),(Xt,Cf,YZeb),(Xt,Cf,YZec),(Xt,Cf,YZed),(Xt,Cf, YZee),(Xt,Cf,YZef),(Xt,Cf,YZeg),(Xt,Cf,YZeh),(Xt,Cf,YZei),(Xt,Cf,YZej),(Xt,Cf,YZek), (Xt,Cf,YZel),(Xt,Cf,YZem),(Xt,Cf,YZen),(Xt,Cf,YZeo),(Xt,Cf,YZep),(Xt,Cf,YZeq), (Xt,Cf,YZer),(Xt,Cf,YZes),(Xt,Cf,YZet),(Xt,Cf,YZeu),(Xt,Cf,YZev),(Xt,Cf,YZew), (Xt,Cf,YZex),(Xt,Cf,YZey),(Xt,Cf,YZez),(Xt,Cf,YZfa),(Xt,Cf,YZfb),(Xt,Cf,YZfc),(Xt, Cf,YZfd),(Xt,Cf,YZfe),(Xt,Cf,YZff),(Xt,Cf,YZfg),(Xt,Cf,YZfhl),(Xt,Cf,YZfl),(Xt,Cf, YZfj),(Xt,Cf,YZfk),(Xt,Cf,YZfl),(Xt,Cf,YZfmn),(Xt,Cf,YZfn),(Xt,Cf,YZfo),(Xt,Cf,YZfp), (Xt,Cf,YZfq),(Xt,Cf,YZfr),(Xt,Cf,YZfs),(Xt,Cf,YZft),(Xt,Cf,YZfu),(Xt,Cf,YZfv),(Xt, Cf,YZfw),(Xt,Cf,YZfx),(Xt,Cf,YZfy),(Xt,Cf,YZfz),(Xt,Cf,YZga),(Xt,Cf,YZgb),(Xt, Cf,YZge),(Xt,Cf,YZgd),(Xt,Cf,YZge),(Xt,Cf,YZgf),(Xt,Cf,YZgg),(Xt,Cf,YZgh),(Xt, Cf,YZgi),(Xt,Cf,YZgj),(Xt,Cf,YZgk),(Xt,Cf,YZgl),(Xt,Cf,YZgm),(Xt,Cf,YZgn),(Xt,Cf, YZgo),(Xt,Cf,YZgp),(Xt,Cf,YZgq),(Xt,Cf,YZgr),(Xt,Cf,YZgs),(Xt,Cf,YZgt), (Xu,Ca,YZa),(Xu,Ca,YZb),(Xu,Ca,YZc),(Xu,Ca,YZd),(Xu,Ca,YZe),(Xu,Ca,YZf),(Xu, Ca,YZg),(Xu,Ca,YZh),(Xu,Ca,YZi),(Xu,Ca,YZj),(Xu,Ca,YZk),(Xu,Ca,YZl),(Xu,Ca,YZm), (Xu,Ca,YZn),(Xu,Ca,YZo),(Xu,Ca,YZp),(Xu,Ca,YZq),(Xu,Ca,YZr),(Xu,Ca,YZs), (Xu,Ca,YZt),(Xu,Ca,YZu),(Xu,Ca,YZv),(Xu,Ca,YZw),(Xu,Ca,YZx),(Xu,Ca,YZy),(Xu, Ca,YZz),(Xu,Ca,YZaa),(Xu,Ca,YZab),(Xu,Ca,YZac),(Xu,Ca,YZad),(Xu,Ca,YZae),(Xu, Ca,YZaf),(Xu,Ca,YZag),(Xu,Ca,YZah),(Xu,Ca,YZai),(Xu,Ca,YZaj),(Xu,Ca,YZak), (Xu,Ca,YZal),(Xu,Ca,YZam),(Xu,Ca,YZan),(Xu,Ca,YZao),(Xu,Ca,YZap),(Xu,Ca,YZaq), (Xu,Ca,YZar),(Xu,Ca,YZas),(Xu,Ca,YZat),(Xu,Ca,YZau),(Xu,Ca,YZav),(Xu,Ca,YZaw), (Xu,Ca,YZax),(Xu,Ca,YZay),(Xu,Ca,YZaz),(Xu,Ca,YZba),(Xu,Ca,YZbb),(Xu, Ca,YZbc),(Xu,Ca,YZbd),(Xu,Ca,YZbe),(Xu,Ca,YZbf),(Xu,Ca,YZbg),(Xu,Ca,YZbh),(Xu, Ca,YZbi),(Xu,Ca,YZbj),(Xu,Ca,YZbk),(Xu,Ca,YZbl),(Xu,Ca,YZbm),(Xu,Ca,YZbn), (Xu,Ca,YZbo),(Xu,Ca,YZbp),(Xu,Ca,YZbq),(Xu,Ca,YZbr),(Xu,Ca,YZbs),(Xu,Ca, YZbt),(Xu,Ca,YZbu),(Xu,Ca,YZbv),(Xu,Ca,YZbw),(Xu,Ca,YZbx),(Xu,Ca,YZby),(Xu, Ca,YZbz),(Xu,Ca,YZca),(Xu,Ca,YZcb),(Xu,Ca,YZcc),(Xu,Ca,YZcd),(Xu,Ca,YZce),(Xu, Ca,YZcf),(Xu,Ca,YZcg),(Xu,Ca,YZch),(Xu,Ca,YZci),(Xu,Ca,YZcj),(Xu,Ca,YZck), (Xu,Ca,YZcl),(Xu,Ca,YZcm),(Xu,Ca,YZcn),(Xu,Ca,YZco),(Xu,Ca,YZcp),(Xu,Ca,YZcq), (Xu,Ca,YZcr),(Xu,Ca,YZcs),(Xu,Ca,YZct),(Xu,Ca,YZcu),(Xu,Ca,YZcv),(Xu,Ca, YZcw),(Xu,Ca,YZcx),(Xu,Ca,YZcy),(Xu,Ca,YZcz),(Xu,Ca,YZda),(Xu,Ca,YZdb),(Xu, Ca,YZdc),(Xu,Ca,YZdd),(Xu,Ca,YZde),(Xu,Ca,YZdf),(Xu,Ca,YZdg),(Xu,Ca,YZdh),(Xu, Ca,YZdi),(Xu,Ca,YZdj),(Xu,Ca,YZdk),(Xu,Ca,YZdl),(Xu,Ca,YZdm),(Xu,Ca,YZdn), (Xu,Ca,YZdo),(Xu,Ca,YZdp),(Xu,Ca,YZdq),(Xu,Ca,YZdr),(Xu,Ca,YZds),(Xu,Ca, YZdt),(Xu,Ca,YZdu),(Xu,Ca,YZdv),(Xu,Ca,YZdw),(Xu,Ca,YZdx),(Xu,Ca,YZdy),(Xu, Ca,YZdz),(Xu,Ca,YZea),(Xu,Ca,YZeb),(Xu,Ca,YZec),(Xu,Ca,YZed),(Xu,Ca,YZee),(Xu, Ca,YZef),(Xu,Ca,YZeg),(Xu,Ca,YZeb),(Xu,Ca,YZei),(Xu,Ca,YZej),(Xu,Ca,YZek), (Xu,Ca,YZel),(Xu,Ca,YZem),(Xu,Ca,YZen),(Xu,Ca,YZeo),(Xu,Ca,YZep),(Xu,Ca,YZeq), (Xu,Ca,YZer),(Xu,Ca,YZes),(Xu,Ca,YZet),(Xu,Ca,YZeu),(Xu,Ca,YZev),(Xu,Ca, YZew),(Xu,Ca,YZex),(Xu,Ca,YZey),(Xu,Ca,YZez),(Xu,Ca,YZfa),(Xu,Ca,YZfb),(Xu, Ca,YZfc),(Xu,Ca,YZfd),(Xu,Ca,YZfe),(Xu,Ca,YZff),(Xu,Ca,YZfg),(Xu,Ca,YZfh),(Xu, Ca,YZfi),(Xu,Ca,YZfj),(Xu,Ca,YZfkc),(Xu,Ca,YZfl),(Xu,Ca,YZfm),(Xu,Ca,YZfn),(Xu, Ca,YZfo),(Xu,Ca,YZfp),(Xu,Ca,YZfq),(Xu,Ca,YZfr),(Xu,Ca,YZfs),(Xu,Ca,YZft),(Xu, Ca,YZfu),(Xu,Ca,YZfv),(Xu,Ca,YZfw),(Xu,Ca,YZfx),(Xu,Ca,YZfy),(Xu,Ca,YZfz),(Xu, Ca,YZga),(Xu,Ca,YZgb),(Xu,Ca,YZgc),(Xu,Ca,YZgd),(Xu,Ca,YZge),(Xu,Ca,YZgf), (Xu,Ca,YZgg),(Xu,Ca,YZgh),(Xu,Ca,YZgi),(Xu,Ca,YZgj),(Xu,Ca,YZgk),(Xu,Ca,YZgl), (Xu,Ca,YZgm),(Xu,Ca,YZgn),(Xu,Ca,YZgo),(Xu,Ca,YZgp),(Xu,Ca,YZgq),(Xu, Ca,YZgr),(Xu,Ca,YZgs),(Xu,Ca,YZgt),(Xu,Cb,YZa),(Xu,Cb,YZb),(Xu,Cb,YZc),(Xu, Cb,YZd),(Xu,Cb,YZe),(Xu,Cb,YZf),(Xu,Cb,YZg),(Xu,Cb,YZh),(Xu,Cb,YZi),(Xu,Cb, YZj),(Xu,Cb,YZk),(Xu,Cb,YZl),(Xu,Cb,YZm),(Xu,Cb,YZn),(Xu,Cb,YZo),(Xu,Cb,YZp), (Xu,Cb,YZq),(Xu,Cb,YZr),(Xu,Cb,YZs),(Xu,Cb,YZt),(Xu,Cb,YZu),(Xu,Cb,YZv),(Xu, Cb,YZw),(Xu,Cb,YZx),(Xu,Cb,YZy),(Xu,Cb,YZz),(Xu,Cb,YZaa),(Xu,Cb,YZab),(Xu, Cb,YZac),(Xu,Cb,YZad),(Xu,Cb,YZae),(Xu,Cb,YZaf),(Xu,Cb,YZag),(Xu,Cb,YZah), (Xu,Cb,YZai),(Xu,Cb,YZaj),(Xu,Cb,YZak),(Xu,Cb,YZal),(Xu,Cb,YZam),(Xu,Cb, YZan),(Xu,Cb,YZao),(Xu,Cb,YZap),(Xu,Cb,YZaq),(Xu,Cb,YZar),(Xu,Cb,YZas),(Xu, Cb,YZat),(Xu,Cb,YZau),(Xu,Cb,YZav),(Xu,Cb,YZaw),(Xu,Cb,YZax),(Xu,Cb,YZay), (Xu,Cb,YZaz),(Xu,Cb,YZba),(Xu,Cb,YZbb),(Xu,Cb,YZbc),(Xu,Cb,YZbd),(Xu,Cb,YZbe), (Xu,Cb,YZbf),(Xu,Cb,YZbg),(Xu,Cb,YZbh),(Xu,Cb,YZbi),(Xu,Cb,YZbj),(Xu,Cb, YZbk),(Xu,Cb,YZbl),(Xu,Cb,YZbm),(Xu,Cb,YZbn),(Xu,Cb,YZbo),(Xu,Cb,YZbp),(Xu, Cb,YZbq),(Xu,Cb,YZbr),(Xu,Cb,YZbs),(Xu,Cb,YZbt),(Xu,Cb,YZbu),(Xu,Cb,YZbv), (Xu,Cb,YZbw),(Xu,Cb,YZbx),(Xu,Cb,YZby),(Xu,Cb,YZbz),(Xu,Cb,YZca),(Xu,Cb, YZcb),(Xu,Cb,YZcc),(Xu,Cb,YZcd),(Xu,Cb,YZce),(Xu,Cb,YZcf),(Xu,Cb,YZcg),(Xu, Cb,YZch),(Xu,Cb,YZci),(Xu,Cb,YZcj),(Xu,Cb,YZck),(Xu,Cb,YZcl),(Xu,Cb,YZcm),(Xu, Cb,YZcn),(Xu,Cb,YZco),(Xu,Cb,YZcp),(Xu,Cb,YZcq),(Xu,Cb,YZcr),(Xu,Cb,YZcs), (Xu,Cb,YZct),(Xu,Cb,YZcu),(Xu,Cb,YZcv),(Xu,Cb,YZcw),(Xu,Cb,YZcx),(Xu,Cb, YZcy),(Xu,Cb,YZcz),(Xu,Cb,YZda),(Xu,Cb,YZdb),(Xu,Cb,YZdc),(Xu,Cb,YZdd),(Xu, Cb,YZde),(Xu,Cb,YZdf),(Xu,Cb,YZdg),(Xu,Cb,YZdh),(Xu,Cb,YZdi),(Xu,Cb,YZdj),(Xu, Cb,YZdk),(Xu,Cb,YZdl),(Xu,Cb,YZdm),(Xu,Cb,YZdn),(Xu,Cb,YZdo),(Xu,Cb,YZdp), (Xu,Cb,YZdq),(Xu,Cb,YZdr),(Xu,Cb,YZds),(Xu,Cb,YZdt),(Xu,Cb,YZdu),(Xu,Cb, YZdv),(Xu,Cb,YZdw),(Xu,Cb,YZdx),(Xu,Cb,YZdy),(Xu,Cb,YZdz),(Xu,Cb,YZea),(Xu, Cb,YZeb), (Xu,Cb,YZec),(Xu,Cb,YZed),(Xu,Cb,YZee),(Xu,Cb,YZef),(Xu,Cb,YZeg), (Xu,Cb,YZeh),(Xu,Cb,YZei),(Xu,Cb,YZej),(Xu,Cb,YZek),(Xu,Cb,YZel),(Xu,Cb,YZem), (Xu,Cb,YZen),(Xu,Cb,YZeo),(Xu,Cb,YZcp),(Xu,Cb,YZeq),(Xu,Cb,YZer),(Xu,Cb, YZes),(Xu,Cb,YZet),(Xu,Cb,YZeu),(Xu,Cb,YZev),(Xu,Cb,YZew),(Xu,Cb,YZex),(Xu, Cb,YZey),(Xu,Cb,YZez),(Xu,Cb,YZfa),(Xu,Cb,YZfb),(Xu,Cb,YZfc),(Xu,Cb,YZfd),(Xu, Cb,YZfe),(Xu,Cb,YZff),(Xu,Cb,YZfg),(Xu,Cb,YZfh),(Xu,Cb,YZfi),(Xu,Cb,YZfj), (Xu,Cb,YZfk),(Xu,Cb,YZfl),(Xu,Cb,YZfm),(Xu,Cb,YZfn),(Xu,Cb,YZfo),(Xu,Cb,YZfp), (Xu,Cb,YZfq),(Xu,Cb,YZfr),(Xu,Cb,YZfs),(Xu,Cb,YZft),(Xu,Cb,YZfu),(Xu,Cb,YZfv), (Xu,Cb,YZfw),(Xu,Cb,YZfx),(Xu,Cb,YZfy),(Xu,Cb,YZfz),(Xu,Cb,YZga),(Xu,Cb, YZgb),(Xu,Cb,YZgc),(Xu,Cb,YZgd),(Xu,Cb,YZge),(Xu,Cb,YZgf),(Xu,Cb,YZgg),(Xu, Cb,YZgb),(Xu,Cb,YZgi),(Xu,Cb,YZgj),(Xu,Cb,YZgk),(Xu,Cb,YZgl),(Xu,Cb,YZgm), (Xu,Cb,YZgn),(Xu,Cb,YZgo),(Xu,Cb,YZgp),(Xu,Cb,YZgq),(Xu,Cb,YZgr),(Xu,Cb,YZgs), (Xu,Cb,YZgt),(Xu,Cc,YZa),(Xu,Cc,YZb),(Xu,Cc,YZc),(Xu,Cc,YZd),(Xu,Cc,YZe), (Xu,Cc,YZf),(Xu,Cc,YZg),(Xu,Cc,YZh),(Xu,Cc,YZi),(Xu,Cc,YZj),(Xu,Cc,YZk),(Xu, Cc,YZl),(Xu,Cc,YZm),(Xu,Cc,YZn),(Xu,Cc,YZo),(Xu,Cc,YZp),(Xu,Cc,YZq),(Xu,Cc, YZr),(Xu,Cc,YZs),(Xu,Cc,YZt),(Xu,Cc,YZu),(Xu,Cc,YZv),(Xu,Cc,YZw),(Xu,Cc,YZx), (Xu,Cc,YZy),(Xu,Cc,YZz),(Xu,Cc,YZaa),(Xu,Cc,YZab),(Xu,Cc,YZac),(Xu,Cc,YZad), (Xu,Cc,YZae),(Xu,Cc,YZaf),(Xu,Cc,YZag),(Xu,Cc,YZah),(Xu,Cc,YZai),(Xu,Ce, YZaj),(Xu,Cc,YZak),(Xu,Cc,YZal),(Xu,Cc,YZam),(Xu,Cc,YZan),(Xu,Cc,YZao),(Xu, Cc,YZap),(Xu,Cc,YZaq),(Xu,Cc,YZar),(Xu,Cc,YZas),(Xu,Cc,YZat),(Xu,Cc,YZau),(Xu, Cc,YZav),(Xu,Cc,YZaw),(Xu,Cc,YZax),(Xu,Cc,YZay),(Xu,Cc,YZaz),(Xu,Cc,YZba), (Xu,Cc,YZbb),(Xu,Cc,YZbc),(Xu,Cc,YZbd),(Xu,Cc,YZbe),(Xu,Cc,YZbf),(Xu,Cc,YZbg), (Xu,Cc,YZbh),(Xu, Cc,YZbi),(Xu,Cc,YZbj),(Xu,Cc,YZbk),(Xu,Cc,YZbl),(Xu,Cc, YZbm),(Xu,Cc,YZbn),(Xu,Cc,YZbo),(Xu,Cc,YZbp),(Xu,Cc,YZbq),(Xu,Cc,YZbr),(Xu, Cc,YZbs),(Xu,Cc,YZbt),(Xu,Cc,YZbu),(Xu,Cc,YZbv),(Xu,Cc,YZbw),(Xu,Cc,YZbx), (Xu,Cc,YZby),(Xu,Cc,YZbz),(Xu,Cc,YZca),(Xu,Cc,YZcb),(Xu,Cc,YZcc),(Xu,Cc,YZcd), (Xu,Cc,YZce),(Xu,Cc,YZcf),(Xu,Cc,YZcg),(Xu,Cc,YZch),(Xu,Cc,YZci),(Xu,Cc, YZcj),(Xu,Cc,YZck),(Xu,Cc,YZcl),(Xu,Cc,YZcm),(Xu,Cc,YZcn),(Xu,Cc,YZco),(Xu, Cc,YZcp),(Xu,Cc,YZcq),(Xu,Cc,YZcr),(Xu,Cc,YZcs),(Xu,Cc,YZct),(Xu,Cc,YZcu),(Xu, Cc,YZcv),(Xu,Cc,YZcw),(Xu,Cc,YZcx),(Xu,Cc,YZcy),(Xu,Cc,YZcz),(Xu,Cc,YZda), (Xu,Cc,YZdb),(Xu,Cc,YZdc),(Xu,Cc,YZdd),(Xu,Cc,YZde),(Xu,Cc,YZdf),(Xu,Cc,YZdg), (Xu,Cc,YZdh),(Xu,Cc,YZdi),(Xu,Cc,YZdj),(Xu,Cc,YZdk),(Xu,Cc,YZdl),(Xu,Cc, YZdm),(Xu,Cc,YZdn),(Xu,Cc,YZdo),(Xu,Cc,YZdp),(Xu,Cc,YZdq),(Xu,Cc,YZdr),(Xu, Cc,YZds),(Xu,Cc,YZdt),(Xu,Cc,YZdu),(Xu,Cc,YZdv),(Xu,Cc,YZdw),(Xu,Cc,YZdx), (Xu,Cc,YZdy),(Xu,Cc,YZdz),(Xu,Cc,YZea),(Xu,Cc,YZeb),(Xu,Cc,YZec),(Xu,Cc,YZed), (Xu,Cc,YZee),(Xu,Cc,YZef),(Xu,Cc,YZeg),(Xu,Cc,YZeh),(Xu,Cc,YZei),(Xu,Cc, YZej),(Xu,Cc,YZek),(Xu,Cc,YZel),(Xu,Cc,YZem),(Xu,Cc,YZen),(Xu,Cc,YZeo),(Xu, Cc,YZep),(Xu,Cc,YZeq),(Xu,Cc,YZer),(Xu,Cc,YZes),(Xu,Cc,YZet),(Xu,Cc,YZeu),(Xu, Cc,YZev),(Xu,Cc,YZew),(Xu,Cc,YZex),(Xu,Cc,YZey),(Xu,Cc,YZez),(Xu,Cc,YZfa), (Xu,Cc,YZfb),(Xu,Cc,YZfc),(Xu,Cc,YZfd),(Xu,Cc,YZfe),(Xu,Cc,YZff),(Xu,Cc,YZfg), (Xu,Cc,YZfh),(Xu,Cc,YZfi),(Xu,Cc,YZfj),(Xu,Cc,YZfk),(Xu,Cc,YZfl),(Xu,Cc,YZfm), (Xu,Cc,YZfn),(Xu,Cc,YZfo),(Xu,Cc,YZfp),(Xu,Cc,YZfq),(Xu,Cc,YZfr),(Xu,Cc,YZfs), (Xu,Cc,YZft),(Xu,Cc,YZfu),(Xu,Cc,YZfv),(Xu,Cc,YZfw),(Xu,Cc,YZfx),(Xu,Cc, YZfy),(Xu,Cc,YZfz),(Xu,Cc,YZga),(Xu,Cc,YZgb),(Xu,Cc,YZgc),(Xu,Cc,YZgd),(Xu, Cc,YZge),(Xu,Cc,YZgf),(Xu,Cc,YZgg),(Xu,Cc,YZgh),(Xu,Cc,YZgi),(Xu,Cc,YZgj),(Xu, Cc,YZgk),(Xu,Cc,YZgl),(Xu,Cc,YZgm),(Xu,Cc,YZgn),(Xu,Cc,YZgo),(Xu,Cc,YZgp), (Xu,Cc,YZgq),(Xu,Cc,YZgr),(Xu,Cc,YZgs),(Xu,Cc,YZgt),(Xu,Cd,YZa),(Xu,Cd,YZb), (Xu,Cd,YZc),(Xu,Cd,YZd),(Xu,Cd,YZe),(Xu,Cd,YZf),(Xu,Cd,YZg),(Xu,Cd,YZh), (Xu,Cd,YZi),(Xu,Cd,YZj),(Xu,Cd,YZk),(Xu,Cd,YZl),(Xu,Cd,YZm),(Xu,Cd,YZn),(Xu, Cd,YZo),(Xu,Cd,YZp),(Xu,Cd,YZq),(Xu,Cd,YZr),(Xu,Cd,YZs),(Xu,Cd,YZt),(Xu,Cd, YZu),(Xu,Cd,YZv),(Xu,Cd,YZw),(Xu,Cd,YZx),(Xu,Cd,YZy),(Xu,Cd,YZz),(Xu,Cd, YZaa),(Xu,Cd,YZab),(Xu,Cd,YZac),(Xu,Cd,YZad),(Xu,Cd,YZae),(Xu,Cd,YZaf),(Xu, Cd,YZag),(Xu,Cd,YZah),(Xu,Cd,YZai),(Xu,Cd,YZaj),(Xu,Cd,YZak),(Xu,Cd,YZal),(Xu, Cd,YZam),(Xu,Cd,YZan),(Xu,Cd,YZao),(Xu,Cd,YZap),(Xu,Cd,YZaq),(Xu,Cd,YZar), (Xu,Cd,YZas),(Xu,Cd,YZat),(Xu,Cd,YZau),(Xu,Cd,YZav),(Xu,Cd,YZaw),(Xu,Cd, YZax),(Xu,Cd,YZay),(Xu,Cd,YZaz),(Xu,Cd,YZba),(Xu,Cd,YZbb),(Xu,Cd,YZbc),(Xu, Cd,YZbd),(Xu,Cd,YZbe),(Xu,Cd,YZbf),(Xu,Cd,YZbg),(Xu,Cd,YZbh),(Xu,Cd,YZbi), (Xu,Cd,YZbj),(Xu,Cd,YZbk),(Xu,Cd,YZbl),(Xu,Cd,YZbm),(Xu,Cd,YZbn),(Xu,Cd,YZbo), (Xu,Cd,YZbp),(Xu,Cd,YZbq),(Xu,Cd,YZbr),(Xu,Cd,YZbs),(Xu,Cd,YZbt),(Xu,Cd, YZbu),(Xu,Cd,YZbv),(Xu,Cd,YZbw),(Xu,Cd,YZbx),(Xu,Cd,YZby),(Xu,Cd,YZbz),(Xu, Cd,YZca),(Xu,Cd,YZcb),(Xu,Cd,YZcc),(Xu,Cd,YZcd),(Xu,Cd,YZce),(Xu,Cd,YZcf), (Xu,Cd,YZcg),(Xu,Cd,YZch),(Xu,Cd,YZci),(Xu,Cd,YZcj),(Xu,Cd,YZck),(Xu,Cd,YZcl), (Xu,Cd,YZcm),(Xu,Cd,YZcn),(Xu,Cd,YZco),(Xu,Cd,YZcp),(Xu,Cd,YZcq),(Xu, Cd,YZcr),(Xu,Cd,YZcs),(Xu,Cd,YZct),(Xu,Cd,YZcu),(Xu,Cd,YZcv),(Xu,Cd,YZcw),(Xu, Cd,YZcx),(Xu,Cd,YZcy),(Xu,Cd,YZcz),(Xu,Cd,YZda),(Xu,Cd,YZdb),(Xu,Cd,YZdc), (Xu,Cd,YZdd),(Xu,Cd,YZde),(X-u,Cd,YZdf),(Xu,Cd,YZdg),(Xu,Cd,YZdh),(Xu,Cd, YZdi),(Xu,Cd,YZdj),(Xu,Cd,YZdk),(Xu,Cd,YZdl),(Xu,Cd,YZdm),(Xu,Cd,YZdn),(Xu, Cd,YZdo),(Xu,Cd,YZdp),(Xu,Cd,YZdq),(Xu,Cd,YZdr),(Xu,Cd,YZds),(Xu,Cd,YZdt), (Xu,Cd,YZdu),(Xu,Cd,YZdv),(Xu,Cd,YZdw),(Xu,Cd,YZdx),(Xu,Cd,YZdy),(Xu,Cd, YZdz),(Xu,Cd,YZea),(Xu,Cd,YZeb),(Xu,Cd,YZec),(Xu,Cd,YZed),(Xu,Cd,YZee),(Xu, Cd,YZef),(Xu,Cd,YZeg),(Xu,Cd,YZeh),(Xu,Cd,YZei) (Xu,Cd,YZej),(Xu,Cd,YZek),(Xu, Cd,YZel),(Xu,Cd,YZem),(Xu,Cd,YZen),(Xu,Cd,YZeo),(Xu,Cd,YZep),(Xu,Cd,YZeq), (Xu,Cd,YZer),(Xu,Cd,YZes),(Xu,Cd,YZet),(Xu,Cd,YZeu),(Xu,Cd,YZev),(Xu,Cd, YZew),(Xu,Cd,YZex),(Xu,Cd,YZey),(Xu,Cd,YZez),(Xu,Cd,YZfa),(Xu,Cd,YZfb),(Xu, Cd,YZfc),(Xu,Cd,YZfd),(Xu,Cd,YZfe),(Xu,Cd,YZff),(Xu,Cd,YZfg),(Xu,Cd,YZfh),(Xu, Cd,YZfi),(Xu,Cd,YZfj),(Xu,Cd,YZfk),(Xu,Cd,YZfl),(Xu,Cd,YZfm),(Xu,Cd,YZfn),(Xu, Cd,YZfo),(Xu,Cd,YZfp),(Xu,Cd,YZfq),(Xu,Cd,YZfr),(Xu,Cd,YZfs),(Xu,Cd,YZft), (Xu,Cd,YZfu),(Xu,Cd,YZfv),(Xu,Cd,YZfw),(Xu,Cd,YZfx),(Xu,Cd,YZfy),(Xu,Cd,YZfz), (Xu,Cd,YZga),(Xu,Cd,YZgb),(Xu,Cd,YZgc),(Xu,Cd,YZgd),(Xu,Cd,YZge),(Xu,Cd, YZgf),(Xu,Cd,YZgg),(Xu,Cd,YZgh),(Xu,Cd,YZgi),(Xu,Cd,YZgj),(Xu,Cd,YZgk),(Xu, Cd,YZgl),(Xu,Cd,YZgm),(Xu,Cd,YZgn),(Xu,Cd,YZgo),(Xu,Cd,YZgp),(Xu,Cd,YZgq), (Xu,Cd,YZgr),(Xu,Cd,YZgs),(Xu,Cd,YZgt),(Xu,Ce,YZa),(Xu,Ce,YZb),(Xu,Ce,YZc), (Xu,Ce,YZd),(Xu,Ce,YZe),(Xu,Ce,YZf),(Xu,Ce,YZg),(Xu,Ce,YZh),(Xu,Ce,YZi),(Xu, Ce,YZj),(Xu,Ce,YZk),(Xu,Ce,YZl),(Xu,Ce,YZm),(Xu,Ce,YZn),(Xu,Ce,YZo),(Xu,Ce, YZp),(Xu,Ce,YZq),(Xu,Ce,YZr),(Xu,Ce,YZs),(Xu,Ce,YZt),(Xu,Ce,YZu),(Xu,Ce,YZv), (Xu,Ce,YZw),(Xu,Ce,YZx),(Xu,Ce,YZy),(Xu,Ce,YZz),(Xu,Ce,YZaa),(Xu,Ce,YZab), (Xu,Ce,YZac),(Xu,Ce,YZad),(Xu,Ce,YZae),(Xu,Ce,YZaf),(Xu,Ce,YZag),(Xu,Ce,YZah), (Xu,Ce,YZai),(Xu,Ce,YZaj),(Xu,Ce,YZak),(Xu,Ce,YZal),(Xu,Ce,YZam),(Xu,Ce, YZan),(Xu,Ce,YZao),(Xu,Ce,YZap),(Xu,Ce,YZaq),(Xu,Ce,YZar),(Xu,Ce,YZas),(Xu, Ce,YZat),(Xu,Ce,YZau),(Xu,Ce,YZav),(Xu,Ce,YZaw),(Xu,Ce,YZax),(Xu,Ce,YZay),(Xu, Ce,YZaz),(Xu,Ce,YZba),(Xu,Ce,YZbb),(Xu,Ce,YZbc),(Xu,Ce,YZbd),(Xu,Ce,YZbe), (Xu,Ce,YZbf),(Xu,Ce,YZbg),(Xu,Ce,YZbh),(Xu,Ce,YZbi),(Xu,Ce,YZbj),(Xu,Ce,YZbk), (Xu,Ce,YZbl),(Xu,Ce,YZbm),(Xu,Ce,YZbn),(Xu,Ce,YZbo),(Xu,Ce,YZbp),(Xu, Ce,YZbq),(Xu,Ce,YZbr),(Xu,Ce,YZbs),(Xu,Ce,YZbt),(Xu,Ce,YZbu),(Xu,Ce,YZbv),(Xu, Ce,YZbw),(Xu,Ce,YZbx),(Xu,Ce,YZby),(Xu,Ce,YZbz),(Xu,Ce,YZca),(Xu,Ce,YZcb), (Xu,Ce,YZcc),(Xu,Ce,YZcd),(Xu,Ce,YZce),(Xu,Ce,YZcf),(Xu,CC,YZcg),(Xu,Ce, YZch),(Xu,Ce,YZci),(Xu,Ce,YZcj),(Xu,Ce,YZck),(Xu,Ce,YZcl),(Xu,Ce,YZcm),(Xu, Ce,YZcn),(Xu,Ce,YZco),(Xu,Ce,YZcp),(Xu,Ce,YZcq),(Xu,Ce,YZcr),(Xu,Ce,YZcs),(Xu, Ce,YZct),(Xu,Ce,YZcu),(Xu,Ce,YZcv),(Xu,Ce,YZcw),(Xu,Ce,YZcx),(Xu,Ce,YZcy), (Xu,Ce,YZcz),(Xu,Ce,YZda),(Xu,Ce,YZdb),(Xu,Ce,YZdc),(Xu,Ce,YZdd),(Xu,Ce, YZde),(Xu,Ce,YZdf),(Xu,Ce,YZdg),(Xu,Ce,YZdh),(Xu,Ce,YZdi),(Xu,Ce,YZdj),(Xu, Ce,YZdk),(Xu,Ce,YZdl),(Xu,Ce,YZdm),(Xu,Ce,YZdn),(Xu,Ce,YZdo),(Xu,Ce,YZdp), (Xu,Ce,YZdq),(Xu,Ce,YZdr),(Xu,Ce,YZds),(Xu,Ce,YZdt),(Xu,Ce,YZdu),(Xu,Ce,YZdv), (Xu,Ce,YZdw),(Xu,Ce,YZdx),(Xu,Ce,YZdy),(Xu,Ce,YZdz),(Xu,Ce,YZea),(Xu,Ce, YZeb),(Xu,Ce,YZec),(Xu,Ce,YZed),(Xu,Ce,YZee),(Xu,Ce,YZef),(Xu,Ce,YZeg),(Xu, Ce,YZeh),(Xu,Ce,YZei),(Xu,Ce,YZej),(Xu,Ce,YZek),(Xu,Ce,YZel),(Xu,Ce,YZem),(Xu, Ce,YZen),(Xu,Ce,YZeo),(Xu,Ce,YZep),(Xu,Ce,YZeq),(Xu,Ce,YZer),(Xu,Ce,YZes), (Xu,Ce,YZet),(Xu,Ce,YZeu),(Xu,Ce,YZev),(Xu,Ce,YZew),(Xu,Ce,YZex),(Xu,Ce,YZey), (Xu,Ce,YZez),(Xu,Ce,YZfa),(Xu,Ce,YZfb),(Xu,Ce,YZfc),(Xu,Ce,YZfd),(Xu,Ce, YZfe),(Xu,Ce,YZff),(Xu,Ce,YZfg),(Xu,Ce,YZfh),(Xu,Ce,YZfi),(Xu,Ce,YZfj),(Xu,Ce, YZfk),(Xu,Ce,YZfl),(Xu,Ce,YZfm),(Xu,Ce,YZfn),(Xu,Ce,YZfo),(Xu,Ce,YZfp),(Xu,Ce, YZfq),(Xu,Ce,YZfr),(Xu,Ce,YZfs),(Xu,Ce,YZft),(Xu,Ce,YZfu),(Xu,Ce,YZfv),(Xu,Ce, YZfw),(Xu,Ce,YZfx),(Xu,Ce,YZfy),(Xu,Ce,YZfz),(Xu,Ce,YZga),(Xu,Ce,YZgb),(Xu, Ce,YZgc),(Xu,Ce,YZgd),(Xu,Ce,YZge),(Xu,Ce,YZgf),(Xu,Ce,YZgg),(Xu,Ce,YZgh), (Xu,Ce,YZgi),(Xu,Ce,YZgj),(Xu,Ce,YZgk),(Xu,Ce,YZgl),(Xu,Ce,YZgm),(Xu,Ce,YZgn), (Xu,Ce,YZgo),(Xu,Ce,YZgp),(Xu,Ce,YZgq),(Xu,Ce,YZgr),(Xu,Ce,YZgs),(Xu,Ce, YZgt),(Xu,Cf,YZa),(Xu,Cf,YZb),(Xu,Cf,YZc),(Xu,Cf,YZd),(Xu,Cf,YZe),(Xu,Cf,YZf), (Xu,Cf,YZg),(Xu,Cf,YZh),(Xu,Cf,YZi),(Xu,Cf,YZj),(Xu,Cf,YZk),(Xu,Cf,YZl),(Xu,Cf, YZm),(Xu,Cf,YZn),(Xu,Cf,YZo),(Xu,Cf,YZp),(Xu,Cf,YZq),(Xu,Cf,YZr),(Xu,Cf,YZs), (Xu,Cf,YZt),(Xu,Cf,YZu),(Xu,Cf,YZv),(Xu,Cf,YZw),(Xu,Cf,YZx),(Xu,Cf,YZy),(Xu, Cf,YZz),(Xu,Cf,YZaa),(Xu,Cf,YZab),(Xu,Cf,YZac),(Xu,Cf,YZad),(Xu,Cf,YZae),(Xu, Cf,YZaf),(Xu,Cf,YZag),(Xu,Cf,YZah),(Xu,Cf,YZai),(Xu,Cf,YZaj),(Xu,Cf,YZak),(Xu, Cf,YZal),(Xu,Cf,YZam),(Xu,Cf,YZan),(Xu,Cf,YZao),(Xu,Cf,YZap),(Xu,Cf,YZaq),(Xu, Cf,YZar),(Xu,Cf,YZas),(Xu,Cf,YZat),(Xu,Cf,YZau),(Xu,Cf,YZav),(Xu,Cf,YZaw), (Xu,Cf,YZax),(Xu,Cf,YZay),(Xu,Cf,YZaz),(Xu,Cf,YZba),(Xu,Cf,YZbb),(Xu,Cf,YZbc), (Xu,Cf,YZbd),(Xu,Cf,YZbe),(Xu,Cf,YZbf),(Xu,Cf,YZbg),(Xu,Cf,YZbh),(Xu,Cf,YZbi), (Xu,Cf,YZbj),(Xu,Cf,YZbk),(Xu,Cf,YZbl),(Xu,Cf,YZbm),(Xu,Cf,YZbn),(Xu,Cf,YZbo), (Xu,Cf,YZbp),(Xu,Cf,YZbq),(Xu,Cf,YZbr),(Xu,Cf,YZbs),(Xu,Cf,YZbt),(Xu,Cf, YZbu),(Xu,Cf,YZbv),(Xu,Cf,YZbw),(Xu,Cf,YZbx),(Xu,Cf,YZby),(Xu,Cf,YZbz),(Xu, Cf,YZca),(Xu,Cf,YZcb),(Xu,Cf,YZcc),(Xu,Cf,YZcd),(Xu,Cf,YZce),(Xu,Cf,YZcf),(Xu, Cf,YZcg),(Xu,Cf,YZch),(Xu,Cf,YZci),(Xu,Cf,YZcj),(Xu,Cf,YZck),(Xu,Cf,YZcl),(Xu, Cf,YZcm),(Xu,Cf,YZcn),(Xu,Cf,YZco),(Xu,Cf,YZcp),(Xu,Cf,YZcq),(Xu,Cf,YZcr),(Xu, Cf,YZcs),(Xu,Cf,YZct),(Xu,Cf,YZcu),(Xu,Cf,YZcv),(Xu,Cf,YZcw),(Xu,Cf,YZcx), (Xu,Cf,YZcy),(Xu,Cf,YZcz),(Xu,Cf,YZda),(Xu,Cf,YZdb),(Xu,Cf,YZdc),(Xu,Cf,YZdd), (Xu,Cf,YZde),(Xu,Cf,YZdf),(Xu,Cf,YZdg),(Xu,Cf,YZdh),(Xu,Cf,YZdi),(Xu,Cf,YZdj), (Xu,Cf,YZdk),(Xu,Cf,YZdl),(Xu,Cf,YZdm),(Xu,Cf,YZdn),(Xu,Cf,YZdo),(Xu,Cf, YZdp),(Xu,Cf,YZdq),(Xu,Cf,YZdr),(Xu,Cf,YZds),(Xu,Cf,YZdt),(Xu,Cf,YZdu),(Xu,Cf, YZdv),(Xu,Cf,YZdw),(Xu,Cf,YZdx),(Xu,Cf,YZdy),(Xu,Cf,YZdz),(Xu,Cf,YZea),(Xu, Cf,YZeb),(Xu,Cf,YZec),(Xu,Cf,YZed),(Xu,Cf,YZee),(Xu,Cf,YZef),(Xu,Cf,YZeg),(Xu, Cf,YZeh),(Xu,Cf,YZei),(Xu,Cf,YZej),(Xu,Cf,YZek),(Xu,Cf,YZel),(Xu,Cf,YZem),(Xu, Cf,YZen),(Xu,Cf,YZeo),(Xu,Cf,YZep),(Xu,Cf,YZeq),(Xu,Cf,YZer),(Xu,Cf,YZes),(Xu, Cf,YZet),(Xu,Cf,YZeu),(Xu,Cf,YZev),(Xu,Cf,YZew),(Xu,Cf,YZex),(Xu,Cf,YZey), (Xu,Cf,YZez),(Xu,Cf,YZfa),(Xu,Cf,YZfb),(Xu,Cf,YZfc),(Xu,Cf,YZfd),(Xu,Cf,YZfe), (Xu,Cf,YZff),(Xu,Cf,YZfg),(Xu,Cf,YZfh),(Xu,Cf,YZfi),(Xu,Cf,YZfj),(Xu,Cf,YZfk),(Xu, Cf,YZfl),(Xu,Cf,YZfm),(Xu,Cf,YZfn),(Xu,Cf,YZfo),(Xu,Cf,YZfp),(Xu,Cf,YZfq),(Xu, Cf,YZfr),(Xu,Cf,YZfs),(Xu,Cf,YZft),(Xu,Cf,YZfu),(Xu,Cf,YZfv),(Xu,Cf,YZfw),(Xu, Cf,YZfx),(Xu,Cf,YZfy),(Xu,Cf,YZfz),(Xu,Cf,YZga),(Xu,Cf,YZgb),(Xu,Cf,YZgc), (Xu,Cf,YZgd),(Xu,Cf,YZge),(Xu,Cf,YZgf),(Xu,Cf,YZgg),(Xu,Cf,YZgh),(Xu,Cf,YZgi), (Xu,Cf,YZgj),(Xu,Cf,YZgk),(Xu,Cf,YZgl),(Xu,Cf,YZgm),(Xu,Cf,YZgn),(Xu,Cf,YZgo), (Xu,Cf,YZgp),(Xu,Cf,YZgq),(Xu,Cf,YZgr),(Xu,Cf,YZgs),(Xu,Cf,YZgt), (Xv,Ca,YZa),(Xv,Ca,YZb),(Xv,Ca,YZc),(Xv,Ca,YZd),(Xv,Ca,YZe),(Xv,Ca,YZf),(Xv, Ca,YZg),(Xv,Ca,YZh),(Xv,Ca,YZi),(Xv,Ca,YZj),(Xv,Ca,YZk),(Xv,Ca,YZl),(Xv,Ca,YZm), (Xv,Ca,YZn),(Xv,Ca,YZo),(Xv,Ca,YZp),(Xv,Ca,YZq),(Xv,Ca,YZr),(Xv,Ca,YZs), (Xv,Ca,YZt),(Xv,Ca,YZu),(Xv,Ca,YZv),(Xv,Ca,YZw),(Xv,Ca,YZx),(Xv,Ca,YZy),(Xv, Ca,YZz),(Xv,Ca,YZaa),(Xv,Ca,YZab),(Xv,Ca,YZac),(Xv,Ca,YZad),(Xv,Ca,YZae),(Xv, Ca,YZaf),(Xv,Ca,YZag),(Xv,Ca,YZah),(Xv,Ca,YZai),(Xv,Ca,YZaj),(Xv,Ca,YZak), (Xv,Ca,YZal),(Xv,Ca,YZam),(Xv,Ca,YZan),(Xv,Ca,YZao),(Xv,Ca,YZap),(Xv,Ca,YZaq), (Xv,Ca,YZar),(Xv,Ca,YZas),(Xv,Ca,YZat),(Xv,Ca,YZau),(Xv,Ca,YZav),(Xv,Ca, YZaw),(Xv,Ca,YZax),(Xv,Ca,YZay),(Xv,Ca,YZaz),(Xv,Ca,YZba),(Xv,Ca,YZbb),(Xv, Ca,YZbc),(Xv,Ca,YZbd),(Xv,Ca,YZbe),(Xv,Ca,YZbf),(Xv,Ca,YZbg),(Xv,Ca,YZbh),(Xv, Ca,YZbi),(Xv,Ca,YZbj),(Xv,Ca,YZbk),(Xv,Ca,YZbl),(Xv,Ca,YZbm),(Xv,Ca,YZbn), (Xv,Ca,YZbo),(Xv,Ca,YZbp),(Xv,Ca,YZbq),(Xv,Ca,YZbr),(Xv,Ca,YZbs),(Xv,Ca, YZbt),(Xv,Ca,YZbu),(Xv,Ca,YZbv),(Xv,Ca,YZbw),(Xv,Ca,YZbx),(Xv,Ca,YZby),(Xv, Ca,YZbz),(Xv,Ca,YZca),(Xv,Ca,YZcb),(Xv,Ca,YZcc),(Xv,Ca,YZcd),(Xv,Ca,YZce),(Xv, Ca,YZcf),(Xv,Ca,YZcg),(Xv,Ca,YZch),(Xv,Ca,YZci),(Xv,Ca,YZcj),(Xv,Ca,YZck), (Xv,Ca,YZcl),(Xv,Ca,YZcm),(Xv,Ca,YZcn),(Xv,Ca,YZco),(Xv,Ca,YZcp),(Xv,Ca,YZcq), (Xv,Ca,YZcr),(Xv,Ca,YZcs),(Xv,Ca,YZct),(Xv,Ca,YZcu),(Xv,Ca,YZcv),(Xv,Ca, YZcw),(Xv,Ca,YZcx),(Xv,Ca,YZcy),(Xv,Ca,YZcz),(Xv,Ca,YZda),(Xv,Ca,YZdb),(Xv, Ca,YZdc),(Xv,Ca,YZdd),(Xv,Ca,YZde),(Xv,Ca,YZdf),(Xv,Ca,YZdg),(Xv,Ca,YZdh),(Xv, Ca,YZdi),(Xv,Ca,YZdj),(Xv,Ca,YZdk),(Xv,Ca,YZdl),(Xv,Ca,YZdm),(Xv,Ca,YZdn), (Xv,Ca,YZdo),(Xv,Ca,YZdp),(Xv,Ca,YZdq),(Xv,Ca,YZdr),(Xv,Ca,YZds),(Xv,Ca, YZdt),(Xv,Ca,YZdu),(Xv,Ca,YZdv),(Xv,Ca,YZdw),(Xv,Ca,YZdx),(Xv,Ca,YZdy),(Xv, Ca,YZdz),(Xv,Ca,YZea),(Xv,Ca,YZeb),(Xv,Ca,YZec),(Xv,Ca,YZed),(Xv,Ca,YZee),(Xv, Ca,YZef),(Xv,Ca,YZeg),(Xv,Ca,YZeh),(Xv,Ca,YZei),(Xv,Ca,YZej),(Xv,Ca,YZek), (Xv,Ca,YZel),(Xv,Ca,YZem),(Xv,Ca,YZen),(Xv,Ca,YZeo),(Xv,Ca,YZep),(Xv,Ca,YZeq), (Xv,Ca,YZer),(Xv,Ca,YZes),(Xv,Ca,YZet),(Xv,Ca,YZeu),(Xv,Ca,YZev),(Xv,Ca, YZew),(Xv,Ca,YZex),(Xv,Ca,YZey),(Xv,Ca,YZez),(Xv,Ca,YZfa),(Xv,Ca,YZfb),(Xv, Ca,YZfc),(Xv,Ca,YZfd),(Xv,Ca,YZfe),(Xv,Ca,YZff),(Xv,Ca,YZfg),(Xv,Ca,YZfh),(Xv, Ca,YZfi),(Xv,Ca,YZfj),(Xv,Ca,YZfk),(Xv,Ca,YZfl),(Xv,Ca,YZfm),(Xv,Ca,YZfn),(Xv, Ca,YZfo),(Xv,Ca,YZfp),(Xv,Ca,YZfq),(Xv,Ca,YZfr),(Xv,Ca,YZfs),(Xv,Ca,YZft),(Xv, Ca,YZfu),(Xv,Ca,YZfv),(Xv,Ca,YZfw),(Xv,Ca,YZfx),(Xv,Ca,YZfy),(Xv,Ca,YZfz),(Xv, Ca,YZga),(Xv,Ca,YZgb),(Xv,Ca,YZgc),(Xv,Ca,YZgd),(Xv,Ca,YZge),(Xv,Ca,YZgf), (Xv,Ca,YZgg),(Xv,Ca,YZgh),(Xv,Ca,YZgi),(Xv,Ca,YZgj),(Xv,Ca,YZgk),(Xv,Ca,YZgl), (Xv,Ca,YZgm),(Xv,Ca,YZgn),(Xv,Ca,YZgo),(Xv,Ca,YZgp),(Xv,Ca,YZgq),(Xv, Ca,YZgr),(Xv,Ca,YZgs),(Xv,Ca,YZgt),(Xv,Cb,YZa),(Xv,Cb,YZb),(Xv,Cb,YZc),(Xv, Cb,YZd),(Xv,Cb,YZe),(Xv,Cb,YZf),(Xv,Cb,YZg),(Xv,Cb,YZh),(Xv,Cb,YZi),(Xv,Cb, YZj),(Xv,Cb,YZk),(Xv,Cb,YZl),(Xv,Cb,YZm),(Xv,Cb,YZn),(Xv,Cb,YZo),(Xv,Cb,YZp), (Xv,Cb,YZq),(Xv,Cb,YZr),(Xv,Cb,YZs),(Xv,Cb,YZt),(Xv,Cb,YZu),(Xv,Cb,YZv),(Xv, Cb,YZw),(Xv,Cb,YZx),(Xv,Cb,YZy),(Xv,Cb,YZz),(Xv,Cb,YZaa),(Xv,Cb,YZab),(Xv, Cb,YZac),(Xv,Cb,YZad),(Xv,Cb,YZae),(Xv,Cb,YZaf),(Xv,Cb,YZag),(Xv,Cb,YZah), (Xv,Cb,YZai),(Xv,Cb,YZaj),(Xv,Cb,YZak),(Xv,Cb,YZal),(Xv,Cb,YZam),(Xv,Cb, YZan),(Xv,Cb,YZao),(Xv,Cb,YZap),(Xv,Cb,YZaq),(Xv,Cb,YZar),(Xv,Cb,YZas),(Xv, Cb,YZat),(Xv,Cb,YZau),(Xv,Cb,YZav),(Xv,Cb,YZaw),(Xv,Cb,YZax),(Xv,Cb,YZay), (Xv,Cb,YZaz),(Xv,Cb,YZba),(Xv,Cb,YZbb),(Xv,Cb,YZbc),(Xv,Cb,YZbd),(Xv,Cb,YZbe), (Xv,Cb,YZbf),(Xv,Cb,YZbg),(Xv,Cb,YZbh),(Xv,Cb,YZbi),(Xv,Cb,YZbj),(Xv,Cb, YZbk),(Xv,Cb,YZbl),(Xv,Cb,YZbm),(Xv,Cb,YZbn),(Xv,Cb,YZbo),(Xv,Cb,YZbp),(Xv, Cb,YZbq),(Xv,Cb,YZbr),(Xv,Cb,YZbs),(Xv,Cb,YZbt),(Xv,Cb,YZbu),(Xv,Cb,YZbv), (Xv,Cb,YZbw),(Xv,Cb,YZbx),(Xv,Cb,YZby),(Xv,Cb,YZbz),(Xv,Cb,YZca),(Xv,Cb, YZcb),(Xv,Cb,YZcc),(Xv,Cb,YZcd),(Xv,Cb,YZce),(Xv,Cb,YZcf),(Xv,Cb,YZcg),(Xv, Cb,YZch),(Xv,Cb,YZci),(Xv,Cb,YZcj),(Xv,Cb,YZck),(Xv,Cb,YZcl),(Xv,Cb,YZcm),(Xv, Cb,YZcn),(Xv,Cb,YZco),(Xv,Cb,YZcp),(Xv,Cb,YZcq),(Xv,Cb,YZcr),(Xv,Cb,YZcs), (Xv,Cb,YZct),(Xv,Cb,YZcu),(Xv,Cb,YZcv),(Xv,Cb,YZcw),(Xv,Cb,YZcx),(Xv,Cb, YZcy),(Xv,Cb,YZcz),(Xv,Cb,YZda),(Xv,Cb,YZdb),(Xv,Cb,YZdc),(Xv,Cb,YZdd),(Xv, Cb,YZde),(Xv,Cb,YZdf),(Xv,Cb,YZdg),(Xv,Cb,YZdh),(Xv,Cb,YZdi),(Xv,Cb,YZdj),(Xv, Cb,YZdk),(Xv,Cb,YZdl),(Xv,Cb,YZdm),(Xv,Cb,YZdn),(Xv,Cb,YZdo),(Xv,Cb,YZdp), (Xv,Cb,YZdq),(Xv,Cb,YZdr),(Xv,Cb,YZds),(Xv,Cb,YZdt),(Xv,Cb,YZdu),(Xv,Cb, YZdv),(Xv,Cb,YZdw),(Xv,Cb,YZdx),(Xv,Cb,YZdy),(Xv,Cb,YZdz),(Xv,Cb,YZea),(Xv, Cb,YZeb),(Xv,Cb,YZec),(Xv,Cb,YZed),(Xv,Cb,YZee),(Xv,Cb,YZef),(Xv,Cb,YZeg), (Xv,Cb,YZeh),(Xv,Cb,YZei),(Xv,Cb,YZej),(Xv,Cb,YZek),(Xv,Cb,YZel),(Xv,Cb,YZem), (Xv,Cb,YZen),(Xv,Cb,YZeo),(Xv,Cb,YZep),(Xv,Cb,YZeq),(Xv,Cb,YZer),(Xv,Cb, YZes),(Xv,Cb,YZet),(Xv,Cb,YZeu),(Xv,Cb,YZev),(Xv,Cb,YZew),(Xv,Cb,YZex),(Xv, Cb,YZey),(Xv,Cb,YZez),(Xv,Cb,YZfa),(Xv,Cb,YZfb),(Xv,Cb,YZfc),(Xv,Cb,YZfd),(Xv, Cb,YZfe),(Xv,Cb,YZff),(Xv,Cb,YZfg),(Xv,Cb,YZfh),(Xv,Cb,YZfi),(Xv,Cb,YZfj), (Xv,Cb,YZfk),(Xv,Cb,YZfl),(Xv,Cb,YZfm),(Xv,Cb,YZfn),(Xv,Cb,YZfo),(Xv,Cb,YZfp), (Xv,Cb,YZfq),(Xv,Cb,YZfr),(Xv,Cb,YZfs),(Xv,Cb,YZft),(Xv,Cb,YZfu),(Xv,Cb,YZfv), (Xv,Cb,YZfw),(Xv,Cb,YZfx),(Xv,Cb,YZfy),(Xv,Cb,YZfz),(Xv,Cb,YZga),(Xv,Cb, YZgb),(Xv,Cb,YZgc),(Xv,Cb,YZgd),(Xv,Cb,YZge),(Xv,Cb,YZgf),(Xv,Cb,YZgg),(Xv, Cb,YZgh),(Xv,Cb,YZgi),(Xv,Cb,YZgj),(Xv,Cb,YZgk),(Xv,Cb,YZgl),(Xv,Cb,YZgm), (Xv,Cb,YZgn),(Xv,Cb,YZgo),(Xv,Cb,YZgp),(Xv,Cb,YZgq),(Xv,Cb,YZgr),(Xv,Cb,YZgs), (Xv,Cb,YZgt),(Xv,Cc,YZa),(Xv,Cc,YZb),(Xv,Cc,YZc),(Xv,Cc,YZd),(Xv,Cc,YZe), (Xv,Cc,YZf),(Xv,Cc,YZg),(Xv,Cc,YZh),(Xv,Cc,YZi),(Xv,Cc,YZj),(Xv,Cc,YZk),(Xv, Cc,YZl),(Xv,Cc,YZm),(Xv,Cc,YZn),(Xv,Cc,YZo),(Xv,Cc,YZp),(Xv,Cc,YZq),(Xv,Cc, YZr),(Xv,Cc,YZs),(Xv,Cc,YZt),(Xv,Cc,YZu),(Xv,Cc,YZv),(Xv,Cc,YZw),(Xv,Cc,YZx), (Xv,Cc,YZy),(Xv,Cc,YZz),(Xv,Cc,YZaa),(Xv,Cc,YZab),(Xv,Cc,YZac),(Xv,Cc,YZad), (Xv,Cc,YZae),(Xv,Cc,YZaf),(Xv,Cc,YZag),(Xv,Cc,YZah),(Xv,Cc,YZai),(Xv,Cc, YZaj),(Xv,Cc,YZak),(Xv,Cc,YZal),(Xv,Cc,YZam),(Xv,Cc,YZan),(Xv,Cc,YZao),(Xv, Cc,YZap),(Xv,Cc,YZaq),(Xv,Cc,YZar),(Xv,Cc,YZas),(Xv,Cc,YZat),(Xv,Cc,YZau),(Xv, Cc,YZav),(Xv,Cc,YZaw),(Xv,Cc,YZax),(Xv,Cc,YZay),(Xv,Cc,YZaz),(Xv,Cc,YZba), (Xv,Cc,YZbb),(Xv,Cc,YZbc),(Xv,Cc,YZbd),(Xv,Cc,YZbe),(Xv,Cc,YZbf),(Xv,Cc,YZbg), (Xv,Cc,YZbh),(Xv,Cc,YZbi),(Xv,Cc,YZbj),(Xv,Cc,YZbk),(Xv,Cc,YZbl),(Xv,Cc, YZbm),(Xv,Cc,YZbn),(Xv,Cc,YZbo),(Xv,Cc,YZbp),(Xv,Cc,YZbq),(Xv,Cc,YZbr),(Xv, Cc,YZbs),(Xv,Cc,YZbt),(Xv,Cc,YZbu),(Xv,Cc,YZbv),(Xv,Cc,YZbw),(Xv,Cc,YZbx), (Xv,Cc,YZby),(Xv,Cc,YZbz),(Xv,Cc,YZca),(Xv,Cc,YZcb),(Xv,Cc,YZcc),(Xv,Cc,YZcd), (Xv,Cc,YZce),(Xv,Cc,YZcf),(Xv,Cc,YZcg),(Xv,Cc,YZch),(Xv,Cc,YZci),(Xv,Cc, YZcj),(Xv,Cc,YZck),(Xv,Cc,YZcl),(Xv,Cc,YZcm),(Xv,Cc,YZcn),(Xv,Cc,YZco),(Xv, Cc,YZcp),(Xv,Cc,YZcq),(Xv,Cc,YZcr),(Xv,Cc,YZcs),(Xv,Cc,YZct),(Xv,Cc,YZcu),(Xv, Cc,YZcv),(Xv,Cc,YZcw),(Xv,Cc,YZcx),(Xv,Cc,YZcy),(Xv,Cc,YZcz),(Xv,Cc,YZda), (Xv,Cc,YZdb),(Xv,Cc,YZdc),(Xv,Cc,YZdd),(Xv,Cc,YZde),(Xv,Cc,YZdf),(Xv,Cc,YZdg), (Xv,Cc,YZdh),(Xv,Cc,YZdi),(Xv,Cc,YZdj),(Xv,Cc,YZdk),(Xv,CG,YZdl),(Xv,Cc, YZdm),(Xv,Ce,YZdn),(Xv,Cc,YZdo),(Xv,Cc,YZdp),(Xv,Cc,YZdq),(Xv,Cc,YZdr),(Xv, Cc,YZds),(Xv,Cc,YZdt),(Xv,Cc,YZdu),(Xv,Cc,YZdv),(Xv,Cc,YZdw),(Xv,Cc,YZdx), (Xv,Cc,YZdy),(Xv,Cc,YZdz),(Xv,Cc,YZea),(Xv,Cc,YZeb),(Xv,Cc,YZec),(Xv,Cc,YZed), (Xv,Cc,YZee),(Xv,Cc,YZef),(Xv,Cc,YZeg),(Xv,Cc,YZeh),(Xv,Cc,YZei),(Xv,Cc, YZej),(Xv,Cc,YZek),(Xv,Cc,YZel),(Xv,Cc,YZem),(Xv,Cc,YZen),(Xv,Cc,YZeo),(Xv, Cc,YZep),(Xv,Cc,YZeq),(Xv,Cc,YZer),(Xv,Cc,YZes),(Xv,Cc,YZet),(Xv,Cc,YZeu),(Xv, Cc,YZev),(Xv,Cc,YZew),(Xv,Cc,YZex),(Xv,Cc,YZey),(Xv,Cc,YZez),(Xv,Cc,YZfa), (Xv,Cc,YZfb),(Xv,Cc,YZfc),(Xv,Cc,YZfd),(Xv,Cc,YZfe),(Xv,Cc,YZff),(Xv,Cc,YZfg), (Xv,Cc,YZfh),(Xv,Cc,YZfi),(Xv,Cc,YZfj),(Xv,Cc,YZfk),(Xv,Cc,YZfl),(Xv,Cc,YZfm), (Xv,Cc,YZfn),(Xv,Cc,YZfo),(Xv,Cc,YZfp),(Xv,Cc,YZfq),(Xv,Cc,YZfr),(Xv,Cc,YZfs), (Xv,Cc,YZft),(Xv,Cc,YZfu),(Xv,Cc,YZfv),(Xv,Cc,YZfw),(Xv,Cc,YZfx),(Xv,Cc, YZfy),(Xv,Cc,YZfz),(Xv,Cc,YZga),(Xv,Cc,YZgb),(Xv,Cc,YZgc),(Xv,Cc,YZgd),(Xv, Cc,YZge),(Xv,Cc,YZgf),(Xv,Cc,YZgg),(Xv,Cc,YZgh),(Xv,Cc,YZgi),(Xv,Cc,YZgj),(Xv, Cc,YZgk),(Xv,Cc,YZgl),(Xv,Cc,YZgm),(Xv,Cc,YZgn),(Xv,Cc,YZgo),(Xv,Cc,YZgp), (Xv,Cc,YZgq),(Xv,Cc,YZgr),(Xv,Cc,YZgs),(Xv,Cc,YZgt),(Xv,Cd,YZa),(Xv,Cd,YZb), (Xv,Cd,YZc),(Xv,Cd,YZd),(Xv,Cd,YZe),(Xv,Cd,YZf),(Xv,Cd,YZg),(Xv,Cd,YZh), (Xv,Cd,YZi),(Xv,Cd,YZj),(Xv,Cd,YZk),(Xv,Cd,YZl),(Xv,Cd,YZm),(Xv,Cd,YZn),(Xv, Cd,YZo),(Xv,Cd,YZp),(Xv,Cd,YZq),(Xv,Cd,YZr),(Xv,Cd,YZs),(Xv,Cd,YZt),(Xv,Cd, YZu),(Xv,Cd,YZv),(Xv,Cd,YZw),(Xv,Cd,YZx),(Xv,Cd,YZy),(Xv,Cd,YZz),(Xv,Cd, YZaa),(Xv,Cd,YZab),(Xv,Cd,YZac),(Xv,Cd,YZad),(Xv,Cd,YZae),(Xv,Cd,YZaf),(Xv, Cd,YZag),(Xv,Cd,YZah),(Xv,Cd,YZai),(Xv,Cd,YZaj),(Xv,Cd,YZak),(Xv,Cd,YZal),(Xv, Cd,YZam),(Xv,Cd,YZan),(Xv,Cd,YZao),(Xv,Cd,YZap),(Xv,Cd,YZaq),(Xv,Cd,YZar), (Xv,Cd,YZas),(Xv,Cd,YZat),(Xv,Cd,YZau),(Xv,Cd,YZav),(Xv,Cd,YZaw),(Xv,Cd, YZax),(Xv,Cd,YZay),(Xv,Cd,YZaz),(Xv,Cd,YZba),(Xv,Cd,YZbb),(Xv,Cd,YZbc),(Xv, Cd,YZbd),(Xv,Cd,YZbe),(Xv,Cd,YZbf),(Xv,Cd,YZbg),(Xv,Cd,YZbh),(Xv,Cd,YZbi), (Xv,Cd,YZbj),(Xv,Cd,YZbk),(Xv,Cd,YZbl),(Xv,Cd,YZbm),(Xv,Cd,YZbn),(Xv,Cd,YZbo), (Xv,Cd,YZbp),(Xv,Cd,YZbq),(Xv,Cd,YZbr),(Xv,Cd,YZbs),(Xv,Cd,YZbt),(Xv,Cd, YZbu),(Xv,Cd,YZbv),(Xv,Cd,YZbw),(Xv,Cd,YZbx),(Xv,Cd,YZby),(Xv,Cd,YZbz),(Xv, Cd,YZca),(Xv,Cd,YZcb),(Xv,Cd,YZcc),(Xv,Cd,YZcd),(Xv,Cd,YZce),(Xv,Cd,YZcf), (Xv,Cd,YZcg),(Xv,Cd,YZch),(Xv,Cd,YZci),(Xv,Cd,YZcj),(Xv,Cd,YZck),(Xv,Cd,YZcl), (Xv,Cd,YZcm),(Xv,Cd,YZcn),(Xv,Cd,YZco),(Xv,Cd,YZcp),(Xv,Cd,YZcq),(Xv, Cd,YZcr),(Xv,Cd,YZcs),(Xv,Cd,YZct),(Xv,Cd,YZcu),(Xv,Cd,YZcv),(Xv,Cd,YZcw),(Xv, Cd,YZcx),(Xv,Cd,YZcy),(Xv,Cd,YZcz),(Xv,Cd,YZda),(Xv,Cd,YZdb),(Xv,Cd,YZdc), (Xv,Cd,YZdd),(Xv,Cd,YZde),(Xv,Cd,YZdf),(Xv,Cd,YZdg),(Xv,Cd,YZdh),(Xv,Cd, YZdi),(Xv,Cd,YZdj),(Xv,Cd,YZdk),(Xv,Cd,YZdl),(Xv,Cd,YZdm),(Xv,Cd,YZdn),(Xv, Cd,YZdo),(Xv,Cd,YZdp),(Xv,Cd,YZdq),(Xv,Cd,YZdr),(Xv,Cd,YZds),(Xv,Cd,YZdt), (Xv,Cd,YZdu),(Xv,Cd,YZdv),(Xv,Cd,YZdw),(Xv,Cd,YZdx),(Xv,Cd,YZdy),(Xv,Cd, YZdz),(Xv,Cd,YZea),(Xv,Cd,YZeb),(Xv,Cd,YZec),(Xv,Cd,YZed),(Xv,Cd,YZee),(Xv, Cd,YZef),(Xv,Cd,YZeg),(Xv,Cd,YZeh),(Xv,Cd,YZei),(Xv,Cd,YZej),(Xv,Cd,YZek),(Xv, Cd,YZel),(Xv,Cd,YZem),(Xv,Cd,YZen),(Xv,Cd,YZeo),(Xv,Cd,YZep),(Xv,Cd,YZeq), (Xv,Cd,YZer),(Xv,Cd,YZes),(Xv,Cd,YZet),(Xv,Cd,YZeu),(Xv,Cd,YZev),(Xv,Cd, YZew),(Xv,Cd,YZex),(Xv,Cd,YZey),(Xv,Cd,YZez),(Xv,Cd,YZfa),(Xv,Cd,YZfb),(Xv, Cd,YZfc),(Xv,Cd,YZfd),(Xv,Cd,YZfe),(Xv,Cd,YZff),(Xv,Cd,YZfg),(Xv,Cd,YZfh),(Xv, Cd,YZfi),(Xv,Cd,YZfj),(Xv,Cd,YZfk),(Xv,Cd,YZfl),(Xv,Cd,YZfm),(Xv,Cd,YZfn),(Xv, Cd,YZfo),(Xv,Cd,YZfp),(Xv,Cd,YZfq),(Xv,Cd,YZfr),(Xv,Cd,YZfs),(Xv,Cd,YZft), (Xv,Cd,YZfu),(Xv,Cd,YZfv),(Xv,Cd,YZfw),(Xv,Cd,YZfx),(Xv,Cd,YZfy),(Xv,Cd,YZfz), (Xv,Cd,YZga),(Xv,Cd,YZgb),(Xv,Cd,YZgc),(Xv,Cd,YZgd),(Xv,Cd,YZge),(Xv,Cd, YZgf),(Xv,Cd,YZgg),(Xv,Cd,YZgh),(Xv,Cd,YZgi),(Xv,Cd,YZgj),(Xv,Cd,YZgk),(Xv, Cd,YZgl),(Xv,Cd,YZgm),(Xv,Cd,YZgn),(Xv,Cd,YZgo),(Xv,Cd,YZgp),(Xv,Cd,YZgq), (Xv,Cd,YZgr),(Xv,Cd,YZgs),(Xv,Cd,YZgt),(Xv,Ce,YZa),(Xv,Ce,YZb),(Xv,Ce,YZc), (Xv,Ce,YZd),(Xv,Ce,YZe),(Xv,Ce,YZf),(Xv,Ce,YZg),(Xv,Ce,YZh),(Xv,Ce,YZi),(Xv, Ce,YZj),(Xv,Ce,YZk),(Xv,Ce,YZl),(Xv,Ce,YZm),(Xv,Ce,YZn),(Xv,Ce,YZo),(Xv,Ce, YZp),(Xv,Ce,YZq),(Xv,Ce,YZr),(Xv,Ce,YZs),(Xv,Ce,YZt),(Xv,Ce,YZu),(Xv,Ce,YZv), (Xv,Ce,YZw),(Xv,Ce,YZx),(Xv,Ce,YZy),(Xv,Ce,YZz),(Xv,Ce,YZaa),(Xv,Ce,YZab), (Xv,Ce,YZac),(Xv,Ce,YZad),(Xv,Ce,YZae),(Xv,Ce,YZaf),(Xv,Ce,YZag),(Xv,Ce,YZah), (Xv,Ce,YZai),(Xv,Ce,YZaj),(Xv,Ce,YZak),(Xv,Ce,YZal),(Xv,Ce,YZam),(Xv,Ce, YZan),(Xv,Ce,YZao),(Xv,Ce,YZap),(Xv,Ce,YZaq),(Xv,Ce,YZar),(Xv,Ce,YZas),(Xv, Ce,YZat),(Xv,Ce,YZau),(Xv,Ce,YZav),(Xv,Ce,YZaw),(Xv,Ce,YZax),(Xv,Ce,YZay),(Xv, Ce,YZaz),(Xv,Ce,YZba),(Xv,Ce,YZbb),(Xv,Ce,YZbc),(Xv,Ce,YZbd),(Xv,Ce,YZbe), (Xv,Ce,YZbf),(Xv,Ce,YZbg),(Xv,Ce,YZbh),(Xv,Ce,YZbi),(Xv,Ce,YZbj),(Xv,Ce,YZbk), (Xv,Ce,YZbl),(Xv,Ce,YZbm),(Xv,Ce,YZbn),(Xv,CC,YZbo),(Xv,Ce,YZbp),(Xv, Cc,YZbq),(Xv,Ce,YZbr),(Xv,Ce,YZbs),(Xv,Ce,YZbt),(Xv,Ce,YZbu),(Xv,Ce,YZbv),(Xv, Ce,YZbw),(Xv,Ce,YZbx),(Xv,Ce,YZby),(Xv,Ce,YZbz),(Xv,Ce,YZca),(Xv,Ce,YZcb), (Xv,Ce,YZcc),(Xv,Ce,YZcd),(Xv,Ce,YZce),(Xv,Ce,YZcf),(Xv,Ce,YZcg),(Xv,Ce, YZch),(Xv,Ce,YZci),(Xv,Ce,YZcj),(Xv,Ce,YZck),(Xv,Ce,YZcl),(Xv,Ce,YZcm),(Xv, Ce,YZcn),(Xv,Ce,YZco),(Xv,Ce,YZcp),(Xv,Ce,YZcq),(Xv,Ce,YZcr),(Xv,Ce,YZcs),(Xv, Ce,YZct),(Xv,Ce,YZcu),(Xv,Ce,YZcv),(Xv,Ce,YZcw),(Xv,Ce,YZcx),(Xv,Ce,YZcy), (Xv,Ce,YZcz),(Xv,Ce,YZda),(Xv,Ce,YZdb),(Xv,Ce,YZdc),(Xv,Ce,YZdd),(Xv,Ce, YZde),(Xv,Ce,YZdf),(Xv,Ce,YZdg),(Xv,Ce,YZdh),(Xv,Ce,YZdi),(Xv,Ce,YZdj),(Xv, Ce,YZdk),(Xv,Ce,YZdl),(Xv,Ce,YZdm),(Xv,Ce,YZdn),(Xv,Ce,YZdo),(Xv,Ce,YZdp), (Xv,Ce,YZdq),(Xv,Ce,YZdr),(Xv,Ce,YZds),(Xv,Ce,YZdt),(Xv,Ce,YZdu),(Xv,Ce,YZdv), (Xv,Ce,YZdw),(Xv,Ce,YZdx),(Xv,Ce,YZdy),(Xv,Ce,YZdz),(Xv,Ce,YZea),(Xv,Ce, YZeb),(Xv,Ce,YZec),(Xv,Ce,YZed),(Xv,Ce,YZee),(Xv,Ce,YZef),(Xv,Ce,YZeg),(Xv, Ce,YZeh),(Xv,Ce,YZei),(Xv,Ce,YZej),(Xv,Ce,YZek),(Xv,Ce,YZel),(Xv,Ce,YZem),(Xv, Ce,YZen),(Xv,Ce,YZeo),(Xv,Ce,YZep),(Xv,Ce,YZeq),(Xv,Ce,YZer),(Xv,Ce,YZes), (Xv,Ce,YZet),(Xv,Ce,YZeu),(Xv,Ce,YZev),(Xv,Ce,YZew),(Xv,Ce,YZex),(Xv,Ce,YZey), (Xv,Ce,YZez),(Xv,Ce,YZfa),(Xv,Ce,YZfb),(Xv,Ce,YZfc),(Xv,Ce,YZfd),(Xv,Ce, YZfe),(Xv,Ce,YZff),(Xv,Ce,YZfg),(Xv,Ce,YZfh),(Xv,Ce,YZfi),(Xv,Ce,YZfj),(Xv,Ce, YZfk),(Xv,Ce,YZfl),(Xv,Ce,YZfm),(Xv,Ce,YZfn),(Xv,Ce,YZfo),(Xv,Ce,YZfp),(Xv,Ce, YZfq),(Xv,Ce,YZfr),(Xv,Ce,YZfs),(Xv,Ce,YZft),(Xv,Ce,YZfu),(Xv,Ce,YZfv),(Xv,Ce, YZfw),(Xv,Ce,YZfx),(Xv,Ce,YZfy),(Xv,Ce,YZfz),(Xv,Ce,YZga),(Xv,Ce,YZgb),(Xv, Ce,YZgc),(Xv,Ce,YZgd),(Xv,Ce,YZge),(Xv,Ce,YZgf),(Xv,Ce,YZgg),(Xv,Ce,YZgh), (Xv,Ce,YZgi),(Xv,Ce,YZgj),(Xv,Ce,YZgk),(Xv,Ce,YZgl),(Xv,Ce,YZgm),(Xv,Ce,YZgn), (Xv,Ce,YZgo),(Xv,Ce,YZgp),(Xv,Ce,YZgq),(Xv,Ce,YZgr),(Xv,Ce,YZgs),(Xv,Ce, YZgt),(Xv,Cf,YZa),(Xv,Cf,YZb),(Xv,Cf,YZc),(Xv,Cf,YZd),(Xv,Cf,YZe),(Xv,Cf,YZf), (Xv,Cf,YZg),(Xv,Cf,YZh),(Xv,Cf,YZi),(Xv,Cf,YZj),(Xv,Cf,YZk),(Xv,Cf,YZl),(Xv,Cf, YZm),(Xv,Cf,YZn),(Xv,Cf,YZo),(Xv,Cf,YZp),(Xv,Cf,YZq),(Xv,Cf,YZr),(Xv,Cf,YZs), (Xv,Cf,YZt),(Xv,Cf,YZu),(Xv,Cf,YZv),(Xv,Cf,YZw),(Xv,Cf,YZx),(Xv,Cf,YZy),(Xv, Cf,YZz),(Xv,Cf,YZaa),(Xv,Cf,YZab),(Xv,Cf,YZac),(Xv,Cf,YZad),(Xv,Cf,YZae),(Xv, Cf,YZaf),(Xv,Cf,YZag),(Xv,Cf,YZah),(Xv,Cf,YZai),(Xv,Cf,YZaj),(Xv,Cf,YZak),(Xv, Cf,YZal),(Xv,Cf,YZam),(Xv,Cf,YZan),(Xv,Cf,YZao),(Xv,Cf,YZap),(Xv,Cf,YZaq),(Xv, Cf,YZar),(Xv,Cf,YZas),(Xv,Cf,YZat),(Xv,Cf,YZau),(Xv,Cf,YZav),(Xv,Cf,YZaw), (Xv,Cf,YZax),(Xv,Cf,YZay),(Xv,Cf,YZaz),(Xv,Cf,YZba),(Xv,Cf,YZbb),(Xv,Cf,YZbc), (Xv,Cf,YZbd),(Xv,Cf,YZbe),(Xv,Cf,YZbf),(Xv,Cf,YZbg),(Xv,Cf,YZbh),(Xv,Cf,YZbi), (Xv,Cf,YZbj),(Xv,Cf,YZbk),(Xv,Cf,YZbl),(Xv,Cf,YZbm),(Xv,Cf,YZbn),(Xv,Cf,YZbo), (Xv,Cf,YZbp),(Xv,Cf,YZbq),(Xv,Cf,YZbr),(Xv,Cf,YZbs),(Xv,Cf,YZbt),(Xv,Cf,YZbu), (Xv,Cf,YZbv),(Xv,Cf,YZbw),(Xv,Cf,YZbx),(Xv,Cf,YZby),(Xv,Cf,YZbz),(Xv, Cf,YZca),(Xv,Cf,YZcb),(Xv,Cf,YZcc),(Xv,Cf,YZcd),(Xv,Cf,YZce),(Xv,Cf,YZcf),(Xv, Cf,YZcg),(Xv,Cf,YZch),(Xv,Cf,YZci),(Xv,Cf,YZcj),(Xv,Cf,YZck),(Xv,Cf,YZcl),(Xv, Cf,YZcm),(Xv,Cf,YZcn),(Xv,Cf,YZco),(Xv,Cf,YZcp),(Xv,Cf,YZcq),(Xv,Cf,YZcr),(Xv, Cf,YZcs),(Xv,Cf,YZct),(Xv,Cf,YZcu),(Xv,Cf,YZcv),(Xv,Cf,YZcw),(Xv,Cf,YZcx), (Xv,Cf,YZcy),(Xv,Cf,YZcz),(Xv,Cf,YZda),(Xv,Cf,YZdb),(Xv,Cf,YZdc),(Xv,Cf,YZdd), (Xv,Cf,YZde),(Xv,Cf,YZdf),(Xv,Cf,YZdg),(Xv,Cf,YZdh),(Xv,Cf,YZdi),(Xv,Cf,YZdj), (Xv,Cf,YZdk),(Xv,Ct,YZdl),(Xv,Cf,YZdm),(Xv,Cf,YZdn),(Xv,Cf,YZdo),(Xv,Cf, YZdp),(Xv,Cf,YZdq),(Xv,Cf,YZdr),(Xv,Cf,YZds),(Xv,Cf,YZdt),(Xv,Cf,YZdu),(Xv,Cf, YZdv),(Xv,Cf,YZdw),(Xv,Cf,YZdx),(Xv,Cf,YZdy),(Xv,Cf,YZdz),(Xv,Cf,YZea),(Xv, Cf,YZeb),(Xv,Cf,YZec),(Xv,Cf,YZed),(Xv,Cf,YZee),(Xv,Cf,YZef),(Xv,Cf,YZeg),(Xv, Cf,YZeh),(Xv,Cf,YZei),(Xv,Cf,YZej),(Xv,Cf,YZek),(Xv,Cf,YZel),(Xv,Cf,YZem),(Xv, Cf,YZen),(Xv,Cf,YZeo),(Xv,Cf,YZep),(Xv,Cf,YZeq),(Xv,Cf,YZer),(Xv,Cf,YZes),(Xv, Cf,YZet),(Xv,Cf,YZeu),(Xv,Cf,YZev),(Xv,Cf,YZew),(Xv,Cf,YZex),(Xv,Cf,YZey), (Xv,Cf,YZez),(Xv,Cf,YZfa),(Xv,Cf,YZfb),(Xv,Cf,YZfc),(Xv,Cf,YZfd),(Xv,Cf,YZfe), (Xv,Cf,YZff),(Xv,Cf,YZfg),(Xv,Cf,YZfh),(Xv,Cf,YZfi),(Xv,Cf,YZfj),(Xv,Cf,YZfk),(Xv, Cf,YZfl),(Xv,Cf,YZfm),(Xv,Cf,YZfn),(Xv,Cf,YZfo),(Xv,Cf,YZfp),(Xv,Cf,YZfq),(Xv, Cf,YZfr),(Xv,Cf,YZfs),(Xv,Cf,YZft),(Xv,Cf,YZfu),(Xv,Cf,YZfv),(Xv,Cf,YZfw),(Xv, Cf,YZfx),(Xv,Cf,YZfy),(Xv,Cf,YZfz),(Xv,Cf,YZga),(Xv,Cf,YZgb),(Xv,Cf,YZgc), (Xv,Cf,YZgd),(Xv,Cf,YZge),(Xv,Cf,YZgf),(Xv,Cf,YZgg),(Xv,Cf,YZgh),(Xv,Cf,YZgi), (Xv,Cf,YZgj),(Xv,Cf,YZgk),(Xv,Cf,YZgl),(Xv,Cf,YZgm),(Xv,Cf,YZgn),(Xv,Cf,YZgo), (Xv,Cf,YZgp),(Xv,Cf,YZgq),(Xv,Cf,YZgr),(Xv,Cf,YZgs),(Xv,Cf,YZgt), (Xw,Ca,YZa),(Xw,Ca,YZb),(Xw,Ca,YZc),(Xw,Ca,YZd),(Xw,Ca,YZe),(Xw,Ca,YZf),(Xw, Ca,YZg),(Xw,Ca,YZh),(Xw,Ca,YZi),(Xw,Ca,YZj),(Xw,Ca,YZk),(Xw,Ca,YZl),(Xw, Ca,YZm),(Xw,Ca,YZn),(Xw,Ca,YZo),(Xw,Ca,YZp),(Xw,Ca,YZq),(Xw,Ca,YZr),(Xw, Ca,YZs),(Xw,Ca,YZt),(Xw,Ca,YZu),(Xw,Ca,YZv),(Xw,Ca,YZw),(Xw,Ca,YZx),(Xw, Ca,YZy),(Xw,Ca,YZz),(Xw,Ca,YZaa),(Xw,Ca,YZab),(Xw,Ca,YZac),(Xw,Ca,YZad), (Xw,Ca,YZae),(Xw,Ca,YZaf),(Xw,Ca,YZag),(Xw,Ca,YZah),(Xw,Ca,YZai),(Xw,Ca, YZaj),(Xw,Ca,YZak),(Xw,Ca,YZal),(Xw,Ca,YZam),(Xw,Ca,YZan),(Xw,Ca,YZao),(Xw, Ca,YZap),(Xw,Ca,YZaq),(Xw,Ca,YZar),(Xw,Ca,YZas),(Xw,Ca,YZat),(Xw,Ca,YZau), (Xw,Ca,YZav),(Xw,Ca,YZaw),(Xw,Ca,YZax),(Xw,Ca,YZay),(Xw,Ca,YZaz),(Xw, Ca,YZba),(Xw,Ca,YZbb),(Xw,Ca,YZbc),(Xw,Ca,YZbd),(Xw,Ca,YZbe),(Xw,Ca,YZbf), (Xw,Ca,YZbg),(Xw,Ca,YZbh),(Xw,Ca,YZbi),(Xw,Ca,YZbj),(Xw,Ca,YZbk),(Xw,Ca, YZbl),(Xw,Ca,YZbm),(Xw,Ca,YZbn),(Xw,Ca,YZbo),(Xw,Ca,YZbp),(Xw,Ca,YZbq), (Xw,Ca,YZbr),(Xw,Ca,YZbs),(Xw,Ca,YZbt),(Xw,Ca,YZbu),(XW,Ca,YZbv),(Xw,Ca, YZbw),(Xw,Ca,YZbx),(Xw,Ca,YZby),(Xw,Ca,YZbz),(Xw,Ca,YZca),(Xw,Ca,YZcb),(Xw, Ca,YZcc),(Xw,Ca,YZcd),(Xw,Ca,YZce),(Xw,Ca,YZcf),(Xw,Ca,YZcg),(Xw,Ca,YZch), (Xw,Ca,YZci),(Xw,Ca,YZcj),(Xw,Ca,YZck),(Xw,Ca,YZcl),(Xw,Ca,YZcm),(Xw, Ca,YZcn),(Xw,Ca,YZco),(Xw,Ca,YZcp),(Xw,Ca,YZcq),(Xw,Ca,YZcr),(Xw,Ca,YZcs), (Xw,Ca,YZct),(Xw,Ca,YZcu),(Xw,Ca,YZcv),(Xw,Ca,YZcw),(Xw,Ca,YZcx),(Xw,Ca, YZcy),(Xw,Ca,YZcz),(Xw,Ca,YZda),(Xw,Ca,YZdb),(Xw,Ca,YZdc),(Xw,Ca,YZdd),(Xw, Ca,YZde),(Xw,Ca,YZdf),(Xw,Ca,YZdg),(Xw,Ca,YZdh),(Xw,Ca,YZdi),(Xw,Ca,YZdj), (Xw,Ca,YZdk),(Xw,Ca,YZdl),(Xw,Ca,YZdm),(Xw,Ca,YZdn),(Xw,Ca,YZdo),(Xw, Ca,YZdp),(Xw,Ca,YZdq),(Xw,Ca,YZdr),(Xw,Ca,YZds),(Xw,Ca,YZdt),(Xw,Ca,YZdu), (Xw,Ca,YZdv),(Xw,Ca,YZdw),(Xw,Ca,YZdx),(Xw,Ca,YZdy),(Xw,Ca,YZdz),(Xw, Ca,YZea),(Xw,Ca,YZeb),(Xw,Ca,YZec),(Xw,Ca,YZed),(Xw,Ca,YZee),(Xw,Ca,YZef), (Xw,Ca,YZeg),(Xw,Ca,YZeh),(Xw,Ca,YZei),(Xw,Ca,YZej),(Xw,Ca,YZek),(Xw,Ca, YZel),(Xw,Ca,YZem),(Xw,Ca,YZen),(Xw,Ca,YZeo),(Xw,Ca,YZep),(Xw,Ca,YZeq), (Xw,Ca,YZer),(Xw,Ca,YZes),(Xw,Ca,YZet),(Xw,Ca,YZeu),(Xw,Ca,YZev),(Xw,Ca,YZew), (Xw,Ca,YZex),(Xw,Ca,YZey),(Xw,Ca,YZez),(Xw,Ca,YZfa),(Xw,Ca,YZfb),(Xw, Ca,YZfc),(Xw,Ca,YZfd),(Xw,Ca,YZfe),(Xw,Ca,YZff),(Xw,Ca,YZfg),(Xw,Ca,YZfh), (Xw,Ca,YZfi),(Xw,Ca,YZfj),(Xw,Ca,YZfk),(Xw,Ca,YZfl),(Xw,Ca,YZfm),(Xw,Ca,YZfn), (Xw,Ca,YZfo),(Xw,Ca,YZfp),(Xw,Ca,YZfq),(Xw,Ca,YZfr),(Xw,Ca,YZfs),(Xw, Ca,YZft),(Xw,Ca,YZfu),(Xw,Ca,YZfv),(Xw,Ca,YZfw),(Xw,Ca,YZfx),(Xw,Ca,YZfy), (Xw,Ca,YZfz),(Xw,Ca,YZga),(Xw,Ca,YZgb),(Xw,Ca,YZgc),(Xw,Ca,YZgd),(Xw,Ca, YZge),(Xw,Ca,YZgf),(Xw,Ca,YZgg),(Xw,Ca,YZgh),(Xw,Ca,YZgi),(Xw,Ca,YZgj),(Xw, Ca,YZgk),(Xw,Ca,YZgl),(Xw,Ca,YZgm),(Xw,Ca,YZgn),(Xw,Ca,YZgo),(Xw,Ca,YZgp), (Xw,Ca,YZgq),(Xw,Ca,YZgr),(Xw,Ca,YZgs),(Xw,Ca,YZgt),(Xw,Cb,YZa),(Xw, Cb,YZb),(Xw,Cb,YZc),(Xw,Cb,YZd),(Xw,Cb,YZe),(Xw,Cb,YZf),(Xw,Cb,YZg),(Xw, Cb,YZh),(Xw,Cb,YZi),(Xw,Cb,YZj),(Xw,Cb,YZk),(Xw,Cb,YZl),(Xw,Cb,YZm),(Xw, Cb,YZn),(Xw,Cb,YZo),(Xw,Cb,YZp),(Xw,Cb,YZq),(Xw,Cb,YZr),(Xw,Cb,YZs),(Xw, Cb,YZt),(Xw,Cb,YZu),(Xw,Cb,YZv),(Xw,Cb,YZw),(Xw,Cb,YZx),(Xw,Cb,YZy),(Xw, Cb,YZz),(Xw,Cb,YZaa),(Xw,Cb,YZab),(Xw,Cb,YZac),(Xw,Cb,YZad),(Xw,Cb,YZae), (Xw,Cb,YZaf),(Xw,Cb,YZag),(Xw,Cb,YZah),(Xw,Cb,YZai),(Xw,Cb,YZaj),(Xw,Cb, YZak),(Xw,Cb,YZal),(Xw,Cb,YZam),(Xw,Cb,YZan),(Xw,Cb,YZao),(Xw,Cb,YZap),(Xw, Cb,YZaq),(Xw,Cb,YZar),(Xw,Cb,YZas),(Xw,Cb,YZat),(Xw,Cb,YZau),(Xw,Cb,YZav), (Xw,Cb,YZaw),(Xw,Cb,YZax),(Xw,Cb,YZay),(Xw,Cb,YZaz),(Xw,Cb,YZba),(Xw, Cb,YZbb),(Xw,Cb,YZbc),(Xw,Cb,YZbd),(Xw,Cb,YZbe),(Xw,Cb,YZbf),(Xw,Cb,YZbg), (Xw,Cb,YZbh),(Xw,Cb,YZbi),(Xw,Cb,YZbj),(Xw,Cb,YZbk),(Xw,Cb,YZbl),(Xw, Cb,YZbm),(Xw,Cb,YZbn),(Xw,Cb,YZbo),(Xw,Cb,YZbp),(Xw,Cb,YZbq),(Xw,Cb, YZbr),(Xw,Cb,YZbs),(Xw,Cb,YZbt),(Xw,Cb,YZbu),(Xw,Cb,YZbv),(Xw,Cb,YZbw),(Xw, Cb,YZbx),(Xw,Cb,YZby),(Xw,Cb,YZbz),(Xw,Cb,YZca),(Xw,Cb,YZcb),(Xw,Cb, YZcc),(Xw,Cb,YZcd),(Xw,Cb,YZce),(Xw,Cb,YZcf),(Xw,Cb,YZcg),(Xw,Cb,YZch),(Xw, Cb,YZci),(Xw,Cb,YZcj),(Xw,Cb,YZck),(Xw,Cb,YZcl),(Xw,Cb,YZcm),(Xw,Cb,YZcn), (Xw,Cb,YZco),(Xw,Cb,YZcp),(Xw,Cb,YZcq),(Xw,Cb,YZcr),(Xw,Cb,YZcs),(Xw, Cb,YZct),(Xw,Cb,YZcu),(Xw,Cb,YZcv),(Xw,Cb,YZcw),(Xw,Cb,YZcx),(Xw,Cb,YZcy), (Xw,Cb,YZcz),(Xw,Cb,YZda),(Xw,Cb,YZdb),(Xw,Cb,YZdc),(Xw,Cb,YZdd),(Xw, Cb,YZde),(Xw,Cb,YZdf),(Xw,Cb,YZdg),(Xw,Cb,YZdh),(Xw,Cb,YZdi),(Xw,Cb,YZdj), (Xw,Cb,YZdk),(Xw,Cb,YZdl),(Xw,Cb,YZdm),(Xw,Cb,YZdn),(Xw,Cb,YZdo),(Xw, Cb,YZdp),(Xw,Cb,YZdq),(Xw,Cb,YZdr),(Xw,Cb,YZds),(Xw,Cb,YZdt),(Xw,Cb,YZdu), (Xw,Cb,YZdv),(Xw,Cb,YZdw),(Xw,Cb,YZdx),(Xw,Cb,YZdy),(Xw,Cb,YZdz),(Xw, Cb,YZea),(Xw,Cb,YZeb),(Xw,Cb,YZec),(Xw,Cb,YZed),(Xw,Cb,YZee),(Xw,Cb,YZef), (Xw,Cb,YZeg),(Xw,Cb,YZeh),(Xw,Cb,YZei),(Xw,Cb,YZej),(Xw,Cb,YZek),(Xw, Cb,YZel),(Xw,Cb,YZem),(Xw,Cb,YZen),(Xw,Cb,YZeo),(Xw,Cb,YZep),(Xw,Cb,YZeq), (Xw,Cb,YZer),(Xw,Cb,YZes),(Xw,Cb,YZet),(Xw,Cb,YZeu),(Xw,Cb,YZev),(Xw, Cb,YZew),(Xw,Cb,YZex),(Xw,Cb,YZey),(Xw,Cb,YZez),(Xw,Cb,YZfa),(Xw,Cb,YZfb), (Xw,Cb,YZfc),(Xw,Cb,YZfd),(Xw,Cb,YZfe),(Xw,Cb,YZff),(Xw,Cb,YZfg),(Xw,Cb, YZfh),(Xw,Cb,YZfi),(Xw,Cb,YZfj),(Xw,Cb,YZfk),(Xw,Cb,YZfl),(Xw,Cb,YZfm),(Xw, Cb,YZfn),(Xw,Cb,YZfo),(Xw,Cb,YZfp),(Xw,Cb,YZfq),(Xw,Cb,YZfr),(Xw,Cb,YZfs), (Xw,Cb,YZft),(Xw,Cb,YZfu),(Xw,Cb,YZfv),(Xw,Cb,YZfw),(Xw,Cb,YZfx),(Xw,Cb, YZfy),(Xw,Cb,YZfz),(Xw,Cb,YZga),(Xw,Cb,YZgb),(Xw,Cb,YZgc),(Xw,Cb,YZgd), (Xw,Cb,YZge),(Xw,Cb,YZgf),(Xw,Cb,YZgg),(Xw,Cb,YZgh),(Xw,Cb,YZgi),(Xw,Cb, YZgj),(Xw,Cb,YZgk),(Xw,Cb,YZgl),(Xw,Cb,YZgm),(Xw,Cb,YZgn),(Xw,Cb,YZgo),(Xw, Cb,YZgp),(Xw,Cb,YZgq),(Xw,Cb,YZgr),(Xw,Cb,YZgs),(Xw,Cb,YZgt),(Xw,Cc, YZa),(Xw,Cc,YZb),(Xw,Cc,YZc),(Xw,Cc,YZd),(Xw,Cc,YZe),(Xw,Cc,YZf),(Xw,Cc, YZg),(Xw,Cc,YZh),(Xw,Cc,YZi),(Xw,Cc,YZj),(Xw,Cc,YZk),(Xw,Cc,YZl),(Xw,Cc,YZm), (Xw,Cc,YZn),(Xw,Cc,YZo),(Xw,Cc,YZp),(Xw,Cc,YZq),(Xw,Cc,YZr),(Xw,Cc,YZs), (Xw,Cc,YZt),(Xw,Cc,YZu),(Xw,Cc,YZv),(Xw,Cc,YZw),(Xw,Cc,YZx),(Xw,Cc,YZy), (Xw,Cc,YZz),(Xw,Cc,YZaa),(Xw,Cc,YZab),(Xw,Cc,YZac),(Xw,Cc,YZad),(Xw, Cc,YZae),(Xw,Cc,YZaf),(Xw,Cc,YZag),(Xw,Cc,YZah),(Xw,Cc,YZai),(Xw,Cc,YZaj), (Xw,Cc,YZak),(Xw,Cc,YZal),(Xw,Cc,YZam),(Xw,Cc,YZan),(Xw,Cc,YZao),(Xw,Cc, YZap),(Xw,Cc,YZaq),(Xw,Cc,YZar),(Xw,Cc,YZas),(Xw,Cc,YZat),(Xw,Cc,YZau),(Xw, Cc,YZav),(Xw,Cc,YZaw),(Xw,Cc,YZax),(Xw,Cc,YZay),(Xw,Cc,YZaz),(Xw,Cc,YZba), (Xw,Cc,YZbb),(Xw,Cc,YZbc),(Xw,Cc,YZbd),(Xw,Cc,YZbe),(Xw,Cc,YZbf),(Xw, Cc,YZbg),(Xw,Cc,YZbh),(Xw,Cc,YZbi),(Xw,Cc,YZbj),(Xw,Cc,YZbk),(Xw,Cc,YZbl), (Xw,Cc,YZbm),(Xw,Cc,YZbn),(Xw,Cc,YZbo),(Xw,Cc,YZbp),(Xw,Cc,YZbq),(Xw, Cc,YZbr),(Xw,Cc,YZbs),(Xw,Cc,YZbt),(Xw,Cc,YZbu),(Xw,Cc,YZbv),(XW,Cc,YZbw), (Xw,Cc,YZbx),(Xw,Cc,YZby),(Xw,Cc,YZbz),(Xw,Cc,YZca),(Xw,Cc,YZcb),(Xw, Cc,YZcc),(Xw,Cc,YZcd),(Xw,Cc,YZce),(Xw,Cc,YZcf),(Xw,Cc,YZcg),(Xw,Cc,YZch), (Xw,Cc,YZci),(Xw,Cc,YZcj),(Xw,Cc,YZck),(Xw,Cc,YZcl),(Xw,Cc,YZcm),(Xw,Cc, YZcn),(Xw,Cc,YZco),(Xw,Cc,YZcp),(Xw,Cc,YZcq),(Xw,Cc,YZcr),(Xw,Cc,YZcs),(Xw, Cc,YZct),(Xw,Cc,YZcu),(Xw,Cc,YZcv),(Xw,Cc,YZcw),(Xw,Cc,YZcx),(Xw,Cc,YZcy), (Xw,Cc,YZcz),(Xw,Cc,YZda),(Xw,Cc,YZdb),(Xw,Cc,YZdc),(Xw,Cc,YZdd),(Xw, Cc,YZde),(Xw,Cc,YZdf),(Xw,Cc,YZdg),(Xw,Cc,YZdh),(Xw,Cc,YZdi),(Xw,Cc,YZdj), (Xw,Cc,YZdk),(Xw,Cc,YZdl),(Xw,Cc,YZdm),(Xw,Cc,YZdn),(Xw,Cc,YZdo),(Xw,Cc, YZdp),(Xw,Cc,YZdq),(Xw,Cc,YZdr),(Xw,Cc,YZds),(Xw,Cc,YZdt),(Xw,Cc,YZdu),(Xw, Cc,YZdv),(Xw,Cc,YZdw),(Xw,Cc,YZdx),(Xw,Cc,YZdy),(Xw,Cc,YZdz),(Xw,Cc, YZea),(Xw,Cc,YZeb),(Xw,Cc,YZec),(Xw,Cc,YZed),(Xw,Cc,YZee),(Xw,Cc,YZef),(Xw, Cc,YZeg),(Xw,Cc,YZeh),(Xw,Cc,YZei),(Xw,Cc,YZej),(Xw,Cc,YZek),(Xw,Cc,YZel), (Xw,Cc,YZem),(Xw,Cc,YZen),(Xw,Cc,YZeo),(Xw,Cc,YZep),(Xw,Cc,YZeq),(Xw, Cc,YZer),(Xw,Cc,YZes),(Xw,Cc,YZet),(Xw,Cc,YZeu),(Xw,Cc,YZev),(Xw,Cc,YZew), (Xw,Cc,YZex),(Xw,Cc,YZey),(Xw,Cc,YZez),(Xw,Cc,YZfa),(Xw,Cc,YZfb),(Xw,Cc, YZfc),(Xw,Cc,YZfd),(Xw,Cc,YZfe),(Xw,Cc,YZff),(Xw,Cc,YZfg),(Xw,Cc,YZfh),(Xw, Cc,YZfi),(Xw,Cc,YZfj),(Xw,Cc,YZfk),(Xw,Cc,YZfl),(Xw,Cc,YZfm),(Xw,Cc,YZfn),(Xw, Cc,YZfo),(Xw,Cc,YZfp),(Xw,Cc,YZfq),(Xw,Cc,YZfr),(Xw,Cc,YZfs),(Xw,Cc,YZft), (Xw,Cc,YZfu),(Xw,Cc,YZfv),(Xw,Cc,YZfw),(Xw,Cc,YZfx),(Xw,Cc,YZfy),(Xw,Cc, YZfz),(Xw,Cc,YZga),(Xw,Cc,YZgb),(Xw,Cc,YZgc),(Xw,Cc,YZgd),(Xw,Cc,YZge), (Xw,Cc,YZgf),(Xw,Cc,YZgg),(Xw,Cc,YZgh),(Xw,Cc,YZgi),(Xw,Cc,YZgj),(Xw,Cc, YZgk),(Xw,Cc,YZgl),(Xw,Cc,YZgm),(Xw,Cc,YZgn),(Xw,Cc,YZgo),(Xw,Cc,YZgp),(Xw, Cc,YZgq),(Xw,Cc,YZgr),(Xw,Cc,YZgs),(Xw,Cc,YZgt),(Xw,Cd,YZa),(Xw,Cd,YZb), (Xw,Cd,YZc),(Xw,Cd,YZd),(Xw,Cd,YZe),(Xw,Cd,YZf),(Xw,Cd,YZg),(Xw,Cd,YZh), (Xw,Cd,YZi),(Xw,Cd,YZj),(Xw,Cd,YZk),(Xw,Cd,YZl),(Xw,Cd,YZm),(Xw,Cd,YZn), (Xw,Cd,YZo),(Xw,Cd,YZp),(Xw,Cd,YZq),(Xw,Cd,YZr),(Xw,Cd,YZs),(Xw,Cd,YZt), (Xw,Cd,YZu),(Xw,Cd,YZv),(Xw,Cd,YZw),(Xw,Cd,YZx),(Xw,Cd,YZy),(Xw,Cd,YZz), (Xw,Cd,YZaa),(Xw,Cd,YZab),(Xw,Cd,YZac),(Xw,Cd,YZad),(Xw,Cd,YZae),(Xw, Cd,YZaf),(Xw,Cd,YZag),(Xw,Cd,YZah),(Xw,Cd,YZai),(Xw,Cd,YZaj),(Xw,Cd,YZak), (Xw,Cd,YZal),(Xw,Cd,YZam),(Xw,Cd,YZan),(Xw,Cd,YZao),(Xw,Cd,YZap),(Xw,Cd, YZaq),(Xw,Cd,YZar),(Xw,Cd,YZas),(Xw,Cd,YZat),(Xw,Cd,YZau),(Xw,Cd,YZav),(Xw, Cd,YZaw),(Xw,Cd,YZax),(Xw,Cd,YZay),(Xw,Cd,YZaz),(Xw,Cd,YZba),(Xw,Cd, YZbb),(Xw,Cd,YZbc),(Xw,Cd,YZbd),(Xw,Cd,YZbe),(Xw,Cd,YZbf),(Xw,Cd,YZbg),(Xw, Cd,YZbh),(Xw,Cd,YZbi),(Xw,Cd,YZbj),(Xw,Cd,YZbk),(Xw,Cd,YZbl),(Xw,Cd,YZbm), (Xw,Cd,YZbn),(Xw,Cd,YZbo),(Xw,Cd,YZbp),(Xw,Cd,YZbq),(Xw,Cd,YZbr),(Xw, Cd,YZbs),(Xw,Cd,YZbt),(Xw,Cd,YZbu),(Xw,Cd,YZbv),(Xw,Cd,YZbw),(Xw,Cd, YZbx),(Xw,Cd,YZby),(Xw,Cd,YZbz),(Xw,Cd,YZca),(Xw,Cd,YZcb),(Xw,Cd,YZcc),(Xw, Cd,YZcd),(Xw,Cd,YZce),(Xw,Cd,YZcf),(Xw,Cd,YZcg),(Xw,Cd,YZch),(Xw,Cd, YZci),(Xw,Cd,YZcj),(Xw,Cd,YZck),(Xw,Cd,YZcl),(Xw,Cd,YZcm),(Xw,Cd,YZcn),(Xw, Cd,YZco),(Xw,Cd,YZcp),(Xw,Cd,YZcq),(Xw,Cd,YZcr),(Xw,Cd,YZcs),(Xw,Cd, YZct),(Xw,Cd,YZcu),(Xw,Cd,YZcv),(Xw,Cd,YZcw),(Xw,Cd,YZcx),(Xw,Cd,YZcy),(Xw, Cd,YZcz),(Xw,Cd,YZda),(Xw,Cd,YZdb),(Xw,Cd,YZdc),(Xw,Cd,YZdd),(Xw,Cd, YZde),(Xw,Cd,YZdf),(Xw,Cd,YZdg),(Xw,Cd,YZdh),(Xw,Cd,YZdi),(Xw,Cd,YZdj),(Xw, Cd,YZdk),(Xw,Cd,YZdl),(Xw,Cd,YZdm),(Xw,Cd,YZdn),(Xw,Cd,YZdo),(Xw,Cd, YZdp),(Xw,Cd,YZdq),(Xw,Cd,YZdr),(Xw,Cd,YZds),(Xw,Cd,YZdt),(Xw,Cd,YZdu),(Xw, Cd,YZdv),(Xw,Cd,YZdw),(Xw,Cd,YZdx),(Xw,Cd,YZdy),(Xw,Cd,YZdz),(Xw,Cd, YZea),(Xw,Cd,YZeb),(Xw,Cd,YZec),(Xw,Cd,YZed),(Xw,Cd,YZee),(Xw,Cd,YZef),(Xw, Cd,YZeg),(Xw,Cd,YZeh),(Xw,Cd,YZei),(Xw,Cd,YZej),(Xw,Cd,YZek),(Xw,Cd,YZel), (Xw,Cd,YZem),(Xw,Cd,YZen),(Xw,Cd,YZeo),(Xw,Cd,YZep),(Xw,Cd,YZeq),(Xw, Cd,YZer),(Xw,Cd,YZes),(Xw,Cd,YZet),(Xw,Cd,YZeu),(Xw,Cd,YZev),(Xw,Cd,YZew), (Xw,Cd,YZex),(Xw,Cd,YZey),(Xw,Cd,YZez),(Xw,Cd,YZfa),(Xw,Cd,YZfb),(Xw, Cd,YZfc),(Xw,Cd,YZfd),(Xw,Cd,YZfe),(Xw,Cd,YZff),(Xw,Cd,YZfg),(Xw,Cd,YZfh), (Xw,Cd,YZfi),(Xw,Cd,YZfj),(Xw,Cd,YZfk),(Xw,Cd,YZfl),(Xw,Cd,YZfm),(Xw,Cd,YZfn), (Xw,Cd,YZfo),(Xw,Cd,YZfp),(Xw,Cd,YZfq),(Xw,Cd,YZfr),(Xw,Cd,YZfs),(Xw, Cd,YZft),(Xw,Cd,YZfu),(Xw,Cd,YZfv),(Xw,Cd,YZfw),(Xw,Cd,YZfx),(Xw,Cd,YZfy), (Xw,Cd,YZfz),(Xw,Cd,YZga),(Xw,Cd,YZgb),(Xw,Cd,YZgc),(Xw,Cd,YZgd),(Xw,Cd, YZge),(Xw,Cd,YZgf),(Xw,Cd,YZgg),(Xw,Cd,YZgh),(Xw,Cd,YZgi),(Xw,Cd,YZgj),(Xw, Cd,YZgk),(Xw,Cd,YZgl),(Xw,Cd,YZgm),(Xw,Cd,YZgn),(Xw,Cd,YZgo),(Xw,Cd, YZgp),(Xw,Cd,YZgq),(Xw,Cd,YZgr),(Xw,Cd,YZgs),(Xw,Cd,YZgt),(Xw,Ce,YZa),(Xw, Ce,YZb),(Xw,Ce,YZc),(Xw,Ce,YZd),(Xw,Ce,YZe),(Xw,Ce,YZf),(Xw,Ce,YZg),(Xw, Ce,YZh),(Xw,Ce,YZi),(Xw,Ce,YZj),(Xw,Ce,YZk),(XW,Ce,YZl),(Xw,Ce,YZm),(Xw, Ce,YZn),(Xw,Ce,YZo),(Xw,Ce,YZp),(Xw,Ce,YZq),(XW,Ce,YZr),(Xw,Ce,YZs),(Xw, Ce,YZt),(Xw,Ce,YZu),(Xw,Ce,YZv),(Xw,Ce,YZw),(Xw,Ce,YZx),(Xw, Ce,YZy),(Xw, Ce,YZz),(Xw,Ce,YZaa),(Xw,Ce,YZab),(Xw,Ce,YZac),(Xw,Ce,YZad),(Xw,Ce,YZae), (Xw,Ce,YZaf),(Xw,Ce,YZag),(Xw,Ce,YZah),(Xw,Ce,YZai),(Xw,Ce,YZaj),(Xw,Ce, YZak),(Xw,Ce,YZal),(Xw,Ce,YZam),(Xw,Ce,YZan),(Xw,Ce,YZao),(Xw,Ce,YZap),(Xw, Ce,YZaq),(Xw,Ce,YZar),(Xw,Ce,YZas),(Xw,Ce,YZat),(Xw,Ce,YZau),(Xw,Ce,YZav), (Xw,Ce,YZaw),(Xw,Ce,YZax),(Xw,Ce,YZay),(Xw,Ce,YZaz),(Xw,Ce,YZba),(Xw, Ce,YZbb),(Xw,Ce,YZbc),(Xw,Ce,YZbd),(Xw,Ce,YZbe),(Xw,Ce,YZbf),(Xw,Ce,YZbg), (Xw,Ce,YZbh),(Xw,Ce,YZbi),(Xw,Ce,YZbj),(Xw,Ce,YZbk),(Xw,Ce,YZbl),(Xw, Ce,YZbm),(Xw,Ce,YZbn),(Xw,Ce,YZbo),(Xw,Ce,YZbp),(Xw,Ce,YZbq),(Xw,Ce,YZbr), (Xw,Ce,YZbs),(Xw,Ce,YZbt),(Xw,Ce,YZbu),(Xw,Ce,YZbv),(Xw,Ce,YZbw),(Xw, Ce,YZbx),(Xw,Ce,YZby),(Xw,Ce,YZbz),(Xw,Ce,YZca),(Xw,Ce,YZcb),(Xw,Ce,YZcc), (Xw,Ce,YZcd),(Xw,Ce,YZce),(Xw,Ce,YZcf),(Xw,Ce,YZcg),(Xw,Ce,YZch),(Xw,Ce, YZci),(Xw,Ce,YZcj),(Xw,Ce,YZck),(Xw,Ce,YZcl),(Xw,Ce,YZcm),(Xw,Ce,YZcn),(Xw, Ce,YZco),(Xw,Ce,YZcp),(Xw,Ce,YZcq),(Xw,Ce,YZcr),(Xw,Ce,YZcs),(Xw,Ce,YZct), (Xw,Ce,YZcu),(Xw,Ce,YZcv),(Xw,Ce,YZcw),(Xw,Ce,YZcx),(Xw,Ce,YZcy),(Xw, Ce,YZcz),(Xw,Ce,YZda),(Xw,Ce,YZdb),(Xw,Ce,YZdc),(Xw,Ce,YZdd),(Xw,Ce,YZde), (Xw,Ce,YZdf),(Xw,Ce,YZdg),(Xw,Ce,YZdh),(Xw,Ce,YZdi),(Xw,Ce,YZdj),(Xw,Ce, YZdk),(Xw,Ce,YZdl),(Xw,Ce,YZdm),(Xw,Ce,YZdn),(Xw,Ce,YZdo),(Xw,Ce,YZdp),(Xw, Ce,YZdq),(Xw,Ce,YZdr),(Xw,Ce,YZds),(Xw,Ce,YZdt),(Xw,Ce,YZdu),(Xw,Ce,YZdv), (Xw,Ce,YZdw),(Xw,Ce,YZdx),(Xw,Ce,YZdy),(Xw,Ce,YZdz),(Xw,Ce,YZea),(Xw, Ce,YZeb),(Xw,Ce,YZec),(Xw,Ce,YZed),(Xw,Ce,YZee),(Xw,Ce,YZef),(Xw,Ce,YZeg), (Xw,Ce,YZeh),(Xw,Ce,YZei),(Xw,Ce,YZej),(Xw,Ce,YZek),(Xw,Ce,YZel),(Xw,Ce, YZem),(Xw,Ce,YZen),(Xw,Ce,YZeo),(Xw,Ce,YZep),(Xw,Ce,YZeq),(Xw,Ce,YZer), (Xw,Ce,YZes),(Xw,Ce,YZet),(Xw,Ce,YZeu),(Xw,Ce,YZev),(Xw,Ce,YZew),(Xw,Ce, YZex),(Xw,Ce,YZey),(Xw,Ce,YZez),(Xw,Ce,YZfa),(Xw,Ce,YZfb),(Xw,Ce,YZfc),(Xw, Ce,YZfd),(Xw,Ce,YZfe),(Xw,Ce,YZff),(Xw,Ce,YZfg),(Xw,Ce,YZfh),(Xw,Ce,YZfi), (Xw,Ce,YZfj),(Xw,Ce,YZfk),(Xw,Ce,YZfl),(Xw,Ce,YZfm),(Xw,Ce,YZfn),(Xw,Ce, YZfo),(Xw,Ce,YZfp),(Xw,Ce,YZfq),(Xw,Ce,YZfr),(Xw,Ce,YZfs),(Xw,Ce,YZft),(Xw, Ce,YZfu),(Xw,Ce,YZfv),(Xw,Ce,YZfw),(Xw,Ce,YZfx),(Xw,Ce,YZfy),(Xw,Ce,YZfz), (Xw,Ce,YZga),(Xw,Ce,YZgb),(Xw,Ce,YZgc),(Xw,Ce,YZgd),(Xw,Ce,YZge),(Xw,Ce, YZgf),(Xw,Ce,YZgg),(Xw,Ce,YZgh),(Xw,Ce,YZgi),(Xw,Cc,YZgj),(Xw,CC,YZgk),(Xw, Ce,YZgl),(Xw,Ce,YZgm),(Xw,Ce,YZgn),(Xw,Ce,YZgo),(Xw,Ce,YZgp),(Xw,Ce,YZgq), (Xw,Ce,YZgr),(Xw,Ce,YZgs),(Xw,Ce,YZgt),(Xw,Cf,YZa),(Xw,Cf,YZb),(Xw,Cf, YZc),(Xw,Cf,YZd),(Xw,Cf,YZe),(Xw,Cf,YZf),(Xw,Cf,YZg),(Xw,Cf,YZh),(Xw,Cf,YZi), (Xw,Cf,YZj),(Xw,Cf,YZk),(Xw,Cf,YZl),(Xw,Cf,YZm),(Xw,Cf,YZn),(Xw,Cf,YZo), (Xw,Cf,YZp),(Xw,Cf,YZq),(Xw,Cf,YZr),(Xw,Cf,YZs),(Xw,Cf,YZt),(Xw,Cf,YZu),(Xw, Cf,YZv),(Xw,Cf,YZw),(Xw,Cf,YZx),(Xw,Cf,YZy),(Xw,Cf,YZz),(Xw,Cf,YZaa),(Xw, Cf,YZab),(Xw,Cf,YZac),(Xw,Cf,YZad),(Xw,Cf,YZae),(Xw,Cf,YZaf),(Xw,Cf,YZag), (Xw,Cf,YZah),(Xw,Cf,YZai),(Xw,Cf,YZaj),(Xw,Cf,YZak),(Xw,Cf,YZal),(Xw,Cf, YZam),(Xw,Cf,YZan),(Xw,Cf,YZao),(Xw,Cf,YZap),(Xw,Cf,YZaq),(Xw,Cf,YZar),(Xw, Cf,YZas),(Xw,Cf,YZat),(Xw,Cf,YZau),(Xw,Cf,YZav),(Xw,Cf,YZaw),(Xw,Cf,YZax), (Xw,Cf,YZay),(Xw,Cf,YZaz),(Xw,Cf,YZba),(Xw,Cf,YZbb),(Xw,Cf,YZbc),(Xw,Cf, YZbd),(Xw,Cf,YZbe),(Xw,Cf,YZbf),(Xw,Cf,YZbg),(Xw,Cf,YZbh),(Xw,Cf,YZbi),(Xw, Cf,YZbj),(Xw,Cf,YZbk),(Xw,Cf,YZbl),(Xw,Cf,YZbm),(Xw,Cf,YZbn),(Xw,Cf,YZbo), (Xw,Cf,YZbp),(Xw,Cf,YZbq),(Xw,Cf,YZbr),(Xw,Cf,YZbs),(Xw,Cf,YZbt),(Xw,Ck, YZbu),(Xw,Cf,YZbv),(Xw,Cf,YZbw),(Xw,Cf,YZbx),(Xw,Cf,YZby),(Xw,Cf,YZbz),(Xw, Cf,YZea),(Xw,Cf,YZeb),(Xw,Cf,YZcc),(Xw,Cf,YZcd),(Xw,Cf,YZce),(Xw,Cf,YZcf), (Xw,Cf,YZcg),(Xw,Cf,YZch),(Xw,Cf,YZci),(Xw,Cf,YZcj),(Xw,Cf,YZck),(Xw,Cf, YZcl),(Xw,Cf,YZcm),(Xw,Cf,YZcn),(Xw,Cf,YZco),(Xw,Cf,YZcp),(Xw,Cf,YZcq),(Xw, Cf,YZcr),(Xw,Cf,YZcs),(Xw,Cf,YZct),(Xw,Cf,YZcu),(Xw,Cf,YZcv),(Xw,Cf,YZcw), (Xw,Cf,YZcx),(Xw,Cf,YZcy),(Xw,Cf,YZcz),(Xw,Cf,YZda),(Xw,Cf,YZdb),(Xw,Cf, YZdc),(Xw,Cf,YZdd),(Xw,Cf,YZde),(Xw,Cf,YZdf),(Xw,Cf,YZdg),(Xw,Cf,YZdh),(Xw, Cf,YZdi),(Xw,Cf,YZdj),(Xw,Cf,YZdk),(Xw,Cf,YZdl),(Xw,Cf,YZdm),(Xw,Cf,YZdn), (Xw,Cf,YZdo),(Xw,Cf,YZdp),(Xw,Cf,YZdq),(Xw,Cf,YZdr),(Xw,Cf,YZds),(Xw,Cf, YZdt),(Xw,Cf,YZdu),(Xw,Cf,YZdv),(Xw,Cf,YZdw),(Xw,Cf,YZdx),(Xw,Cf,YZdy),(Xw, Cf,YZdz),(Xw,Cf,YZea),(Xw,Cf,YZeb),(Xw,Cf,YZec),(Xw,Cf,YZed),(Xw,Cf,YZee), (Xw,Cf,YZef),(Xw,Cf,YZeg),(Xw,Cf,YZeh),(Xw,Cf,YZei),(Xw,Cf,YZej),(Xw,Cf, YZek),(Xw,Cf,YZel),(Xw,Cf,YZem),(Xw,Cf,YZen),(Xw,Cf,YZeo),(Xw,Cf,YZep),(Xw, Cf,YZeq),(Xw,Cf,YZer),(Xw,Cf,YZes),(Xw,Cf,YZet),(Xw,Cf,YZeu),(Xw,Cf,YZev), (Xw,Cf,YZew),(Xw,Cf,YZex),(Xw,Cf,YZey),(Xw,Cf,YZez),(Xw,Cf,YZfa),(Xw,Cf, YZfb),(Xw,Cf,YZfc),(Xw,Cf,YZfd),(Xw,Cf,YZfe),(Xw,Cf,YZff),(Xw,Cf,YZfg),(Xw, Cf,YZfh),(Xw,Cf,YZfi),(Xw,Cf,YZfj),(Xw,Cf,YZfk),(Xw,Cf,YZfl),(Xw,Cf,YZfm),(Xw, Cf,YZfn),(Xw,Cf,YZfo),(Xw,Cf,YZfp),(Xw,Cf,YZfq),(Xw,Cf,YZfr),(Xw,Cf,YZfs), (Xw,Cf,YZft),(Xw,Cf,YZfu),(Xw,Cf,YZfv),(Xw,Cf,YZfw),(Xw,Cf,YZfx),(Xw,Cf,YZfy), (Xw,Cf,YZfz),(Xw,Cf,YZga),(Xw,Cf,YZgb),(Xw,Cf,YZgc),(Xw,Cf,YZgd),(Xw,Cf, YZge),(Xw,Cf,YZgf),(Xw,Cf,YZgg),(Xw,Cf,YZgh),(Xw,Cf,YZgi),(Xw,Cf,YZgj),(Xw, Cf,YZgk),(Xw,Cf,YZgl),(Xw,Cf,YZgm),(Xw,Cf,YZgn),(Xw,Cf,YZgo),(Xw,Cf,YZgp), (Xw,Cf,YZgq),(Xw,Cf,YZgr),(Xw,Cf,YZgs),(Xw,Cf,YZgt), (Xx,Ca,YZa),(Xx,Ca,YZb),(Xx,Ca,YZc),(Xx,Ca,YZd),(Xx,Ca,YZe),(Xx,Ca,YZf),(Xx, Ca,YZg),(Xx,Ca,YZh),(Xx,Ca,YZi),(Xx,Ca,YZj),(YX,Ca,YZk),(Xx,Ca,YZl),(Xx,Ca,YZm), (Xx,Ca,YZn),(Xx,Ca,YZo),(Xx,Ca,YZp),(Xx,Ca,YZq),(Xx,Ca,YZr),(Xx,Ca,YZs), (Xx,Ca,YZt),(Xx,Ca,YZu),(Xx,Ca,YZv),(Xx,Ca,YZw),(Xx,Ca,YZx),(Xx,Ca,YZy),(Xx, Ca,YZz),(Xx,Ca,YZaa),(Xx,Ca,YZab),(Xx,Ca,YZac),(Xx,Ca,YZad),(Xx,Ca,YZae),(Xx, Ca,YZaf),(Xx,Ca,YZag),(Xx,Ca,YZah),(Xx,Ca,YZai),(Xx,Ca,YZaj),(Xx,Ca,YZak), (Xx,Ca,YZal),(Xx,Ca,YZam),(Xx,Ca,YZan),(Xx,Ca,YZao),(Xx,Ca,YZap),(Xx,Ca,YZaq), (Xx,Ca,YZar),(Xx,Ca,YZas),(Xx,Ca,YZat),(Xx,Ca,YZau),(Xx,Ca,YZav),(Xx,Ca, YZaw),(Xx,Ca,YZax),(Xx,Ca,YZay),(Xx,Ca,YZaz),(Xx,Ca,YZba),(Xx,Ca,YZbb),(Xx, Ca,YZbc),(Xx,Ca,YZbd),(Xx,Ca,YZbe),(Xx,Ca,YZbf),(Xx,Ca,YZbg),(Xx,Ca,YZbh),(Xx, Ca,YZbi),(Xx,Ca,YZbj),(Xx,Ca,YZbk),(Xx,Ca,YZbl),(Xx,Ca,YZbm),(Xx,Ca,YZbn), (Xx,Ca,YZbo),(Xx,Ca,YZbp),(Xx,Ca,YZbq),(Xx,Ca,YZbr),(Xx,Ca,YZbs),(Xx,Ca, YZbt),(Xx,Ca,YZbu),(Xx,Ca,YZbv),(Xx,Ca,YZbw),(Xx,Ca,YZbx),(Xx,Ca,YZby),(Xx, Ca,YZbz),(Xx,Ca,YZca),(Xx,Ca,YZcb),(Xx,Ca,YZcc),(Xx,Ca,YZcd),(Xx,Ca,YZce),(Xx, Ca,YZcf),(Xx,Ca,YZcg),(Xx,Ca,YZch),(Xx,Ca,YZci),(Xx,Ca,YZcj),(Xx,Ca,YZck), (Xx,Ca,YZcl),(Xx,Ca,YZcm),(Xx,Ca,YZcn),(Xx,Ca,YZco),(Xx,Ca,YZcp),(Xx,Ca,YZcq), (Xx,Ca,YZcr),(Xx,Ca,YZcs),(Xx,Ca,YZct),(Xx,Ca,YZcu),(Xx,Ca,YZcv),(Xx,Ca, YZcw),(Xx,Ca,YZcx),(Xx,Ca,YZcy),(Xx,Ca,YZcz),(Xx,Ca,YZda),(Xx,Ca,YZdb),(Xx, Ca,YZdc),(Xx,Ca,YZdd),(Xx,Ca,YZde),(Xx,Ca,YZdf),(Xx,Ca,YZdg),(Xx,Ca,YZdh),(Xx, Ca,YZdi),(Xx,Ca,YZdj),(Xx,Ca,YZdk),(Xx,Ca,YZdl),(Xx,Ca,YZdm),(Xx,Ca,YZdn), (Xx,Ca,YZdo),(Xx,Ca,YZdp),(Xx,Ca,YZdq),(Xx,Ca,YZdr),(Xx,Ca,YZds),(Xx,Ca, YZdt),(Xx,Ca,YZdu),(Xx,Ca,YZdv),(Xx,Ca,YZdw),(Xx,Ca,YZdx),(Xx,Ca,YZdy),(Xx, Ca,YZdz),(Xx,Ca,YZea),(Xx,Ca,YZeb),(Xx,Ca,YZec),(Xx,Ca,YZed),(Xx,Ca,YZee),(Xx, Ca,YZef),(Xx,Ca,YZeg),(Xx,Ca,YZeh),(Xx,Ca,YZei),(Xx,Ca,YZej),(Xx,Ca,YZek), (Xx,Ca,YZel),(Xx,Ca,YZem),(Xx,Ca,YZen),(Xx,Ca,YZeo),(Xx,Ca,YZep),(Xx,Ca,YZeq), (Xx,Ca,YZer),(Xx,Ca,YZes),(Xx,Ca,YZet),(Xx,Ca,YZeu),(Xx,Ca,YZev),(Xx,Ca, YZew),(Xx,Ca,YZex),(Xx,Ca,YZey),(Xx,Ca,YZez),(Xx,Ca,YZfa),(Xx,Ca,YZfb),(Xx, Ca,YZfc),(Xx,Ca,YZfd),(Xx,Ca,YZfe),(Xx,Ca,YZff),(Xx,Ca,YZfg),(Xx,Ca,YZfh),(Xx, Ca,YZfi),(Xx,Ca,YZfj),(Xx,Ca,YZfk),(Xx,Ca,YZfl),(Xx,Ca,YZfm),(Xx,Ca,YZfn),(Xx, Ca,YZfo),(Xx,Ca,YZfp),(Xx,Ca,YZfq),(Xx,Ca,YZfr),(Xx,Ca,YZfs),(Xx,Ca,YZft),(Xx, Ca,YZfu),(Xx,Ca,YZfv),(Xx,Ca,YZfw),(Xx,Ca,YZfx),(Xx,Ca,YZfy),(Xx,Ca,YZfz),(Xx, Ca,YZga),(Xx,Ca,YZgb),(Xx,Ca,YZgc),(Xx,Ca,YZgd),(Xx,Ca,YZge),(Xx,Ca,YZgf), (Xx,Ca,YZgg),(Xx,Ca,YZgh),(Xx,Ca,YZgi),(Xx,Ca,YZgj),(Xx,Ca,YZgk),(Xx,Ca,YZgl), (Xx,Ca,YZgm),(Xx,Ca,YZgn),(Xx,Ca,YZgo),(Xx,Ca,YZgp),(Xx,Ca,YZgq),(Xx, Ca,YZgr),(Xx,Ca,YZgs),(Xx,Ca,YZgt),(Xx,Cb,YZa),(Xx,Cb,YZb),(Xx,Cb,YZc),(Xx, Cb,YZd),(Xx,Cb,YZe),(Xx,Cb,YZf),(Xx,Cb,YZg),(Xx,Cb,YZh),(Xx,Cb,YZi),(Xx,Cb, YZj),(Xx,Cb,YZk),(Xx,Cb,YZl),(Xx,Cb,YZm),(Xx,Cb,YZn),(Xx,Cb,YZo),(Xx,Cb,YZp), (Xx,Cb,YZq),(Xx,Cb,YZr),(Xx,Cb,YZs),(Xx,Cb,YZt),(Xx,Cb,YZu),(Xx,Cb,YZv),(Xx, Cb,YZw),(Xx,Cb,YZx),(Xx,Cb,YZy),(Xx,Cb,YZz),(Xx,Cb,YZaa),(Xx,Cb,YZab),(Xx, Cb,YZac),(Xx,Cb,YZad),(Xx,Cb,YZae),(Xx,Cb,YZaf),(Xx,Cb,YZag),(Xx,Cb,YZah), (Xx,Cb,YZai),(Xx,Cb,YZaj),(Xx,Cb,YZak),(Xx,Cb,YZal),(Xx,Cb,YZam),(Xx,Cb, YZan),(Xx,Cb,YZao),(Xx,Cb,YZap),(Xx,Cb,YZaq),(Xx,Cb,YZar),(Xx,Cb,YZas),(Xx, Cb,YZat),(Xx,Cb,YZau),(Xx,Cb,YZav),(Xx,Cb,YZaw),(Xx,Cb,YZax),(Xx,Cb,YZay), (Xx,Cb,YZaz),(Xx,Cb,YZba),(Xx,Cb,YZbb),(Xx,Cb,YZbc),(Xx,Cb,YZbd),(Xx,Cb,YZbe), (Xx,Cb,YZbf),(Xx,Cb,YZbg),(Xx,Cb,YZbh),(Xx,Cb,YZbi),(Xx,Cb,YZbj),(Xx,Cb, YZbk),(Xx,Cb,YZbl),(Xx,Cb,YZbm),(Xx,Cb,YZbn),(Xx,Cb,YZbo),(Xx,Cb,YZbp),(Xx, Cb,YZbq),(Xx,Cb,YZbr),(Xx,Cb,YZbs),(Xx,Cb,YZbt),(Xx,Cb,YZbu),(Xx,Cb,YZbv), (Xx,Cb,YZbw),(Xx,Cb,YZbx),(Xx,Cb,YZby),(Xx,Cb,YZbz),(Xx,Cb,YZca),(Xx,Cb, YZcb),(Xx,Cb,YZcc),(Xx,Cb,YZcd),(Xx,Cb,YZce),(Xx,Cb,YZcf),(Xx,Cb,YZcg),(Xx, Cb,YZch),(Xx,Cb,YZci),(Xx,Cb,YZcj),(Xx,Cb,YZck),(Xx,Cb,YZcl),(Xx,Cb,YZcm),(Xx, Cb,YZcn),(Xx,Cb,YZco),(Xx,Cb,YZcp),(Xx,Cb,YZcq),(Xx,Cb,YZcr),(Xx,Cb,YZcs), (Xx,Cb,YZct),(Xx,Cb,YZcu),(Xx,Cb,YZcv),(Xx,Cb,YZcw),(Xx,Cb,YZcx),(Xx,Cb, YZcy),(Xx,Cb,YZcz),(Xx,Cb,YZda),(Xx,Cb,YZdb),(Xx,Cb,YZdc),(Xx,Cb,YZdd),(Xx, Cb,YZde),(Xx,Cb,YZdf),(Xx,Cb,YZdg),(Xx,Cb,YZdh),(Xx,Cb,YZdi),(Xx,Cb,YZdj),(Xx, Cb,YZdk),(Xx,Cb,YZdl),(Xx,Cb,YZdm),(Xx,Cb,YZdn),(Xx,Cb,YZdo),(Xx,Cb,YZdp), (Xx,Cb,YZdq),(Xx,Cb,YZdr),(Xx,Cb,YZds),(Xx,Cb,YZdt),(Xx,Cb,YZdu),(Xx,Cb, YZdv),(Xx,Cb,YZdw),(Xx,Cb,YZdx),(Xx,Cb,YZdy),(Xx,Cb,YZdz),(Xx,Cb,YZea),(Xx, Cb,YZeb),(Xx,Cb,YZec),(Xx,Cb,YZed),(Xx,Cb,YZee),(Xx,Cb,YZef),(Xx,Cb,YZeg), (Xx,Cb,YZeh),(Xx,Cb,YZei),(Xx,Cb,YZej),(Xx,Cb,YZek),(Xx,Cb,YZel),(Xx,Cb,YZem), (Xx,Cb,YZen),(Xx,Cb,YZeo),(Xx,Cb,YZep),(Xx,Cb,YZeq),(Xx,Cb,YZer),(Xx,Cb, YZes),(Xx,Cb,YZet),(Xx,Cb,YZeu),(Xx,Cb,YZev),(Xx,Cb,YZew),(Xx,Cb,YZex),(Xx, Cb,YZey),(Xx,Cb,YZez),(Xx,Cb,YZfa),(Xx,Cb,YZfb),(Xx,Cb,YZfc),(Xx,Cb,YZfd),(Xx, Cb,YZfe),(Xx,Cb,YZff),(Xx,Cb,YZfg),(Xx,Cb,YZfh),(Xx,Cb,YZfi),(Xx,Cb,YZfj), (Xx,Cb,YZfk),(Xx,Cb,YZfl),(Xx,Cb,YZfm),(Xx,Cb,YZfn),(Xx,Cb,YZfo),(Xx,Cb,YZfp), (Xx,Cb,YZfq),(Xx,Cb,YZfr),(Xx,Cb,YZfs),(Xx,Cb,YZft),(Xx,Cb,YZfu),(Xx,Cb,YZfv), (Xx,Cb,YZfw),(Xx,Cb,YZfx),(Xx,Cb,YZfy),(Xx,Cb,YZfz),(Xx,Cb,YZga),(Xx,Cb, YZgb),(Xx,Cb,YZgc),(Xx,Cb,YZgd),(Xx,Cb,YZge),(Xx,Cb,YZgf),(Xx,Cb,YZgg),(Xx, Cb,YZgh),(Xx,Cb,YZgi),(Xx,Cb,YZgj),(Xx,Cb,YZgk),(Xx,Cb,YZgl),(Xx,Cb,YZgm), (Xx,Cb,YZgn),(Xx,Cb,YZgo),(Xx,Cb,YZgp),(Xx,Cb,YZgq),(Xx,Cb,YZgr),(Xx,Cb,YZgs), (Xx,Cb,YZgt),(Xx,Cc,YZa),(Xx,Cc,YZb),(Xx,Cc,YZc),(Xx,Cc,YZd),(Xx,Cc,YZe), (Xx,Cc,YZf),(Xx,Cc,YZg),(Xx,Cc,YZh),(YXx,Cc,YZi),(Xx,Cc,YZj),(Xx,Cc,YZk),(Xx, Cc,YZl),(Xx,Cc,YZm),(Xx,Cc,YZn),(Xx,Cc,YZo),(Xx,Cc,YZp),(Xx,Cc,YZq),(Xx,Cc, YZr),(Xx,Cc,YZs),(Xx,Cc,YZt),(Xx,Cc,YZu),(Xx,Cc,YZv),(Xx,Cc,YZw),(Xx,Cc,YZx), (Xx,Cc,YZy),(Xx,Cc,YZz),(Xx,Cc,YZaa),(Xx,Cc,YZab),(Xx,Cc,YZac),(Xx,Cc,YZad), (Xx,Cc,YZae),(Xx,Cc,YZaf),(Xx,Cc,YZag),(Xx,Cc,YZah),(Xx,Cc,YZai),(Xx,Cc, YZaj),(Xx,Cc,YZak),(Xx,Cc,YZal),(Xx,Cc,YZam),(Xx,Cc,YZan),(Xx,Cc,YZao),(Xx, Cc,YZap),(Xx,Cc,YZaq),(Xx,Cc,YZar),(Xx,Cc,YZas),(Xx,Cc,YZat),(Xx,Cc,YZau),(Xx, Cc,YZav),(Xx,Cc,YZaw),(Xx,Cc,YZax),(Xx,Cc,YZay),(Xx,Cc,YZaz),(Xx,Cc,YZba), (Xx,Cc,YZbb),(Xx,Cc,YZbc),(Xx,Cc,YZbd),(Xx,Cc,YZbe),(Xx,Cc,YZbf),(Xx,Cc,YZbg), (Xx,Cc,YZbh),(Xx,Cc,YZbi),(Xx,Cc,YZbj),(Xx,Cc,YZbk),(Xx,Cc,YZbl),(Xx,Cc, YZbm),(Xx,Cc,YZbn),(Xx,Cc,YZbo),(Xx,Cc,YZbp),(Xx,Cc,YZbq),(Xx,Cc,YZbr),(Xx, Cc,YZbs),(Xx,Cc,YZbt),(Xx,CC,YZbu),(Xx,Cc,YZbv),(Xx,Cc,YZbw),(Xx,Cc,YZbx), (Xx,Cc,YZby),(Xx,Cc,YZbz),(Xx,Cc,YZca),(Xx,Cc,YZcb),(Xx,Cc,YZcc),(Xx,Cc,YZcd), (Xx,Cc,YZce),(Xx,Cc,YZcf),(Xx,Cc,YZcg),(Xx,Cc,YZch),(Xx,Cc,YZci),(Xx,Cc, YZcj),(Xx,Cc,YZck),(Xx,Cc,YZcl),(Xx,Cc,YZcm),(Xx,Cc,YZcn),(Xx,Cc,YZco),(Xx, Cc,YZcp),(Xx,Cc,YZcq),(Xx,Cc,YZcr),(Xx,Cc,YZcs),(Xx,Cc,YZct),(Xx,Cc,YZcu),(Xx, Cc,YZcv),(Xx,Cc,YZcw),(Xx,Cc,YZcx),(Xx,Cc,YZcy),(Xx,CC,YZcz),(Xx,Cc,YZda), (Xx,Cc,YZdb),(Xx,Cc,YZdc),(Xx,Cc,YZdd),(Xx,Cc,YZde),(Xx,Cc,YZdf),(Xx,Cc,YZdg), (Xx,Cc,YZdh),(Xx,Cc,YZdi),(Xx,Cc,YZdj),(Xx,Cc,YZdk),(Xx,Cc,YZdl),(Xx,Cc, YZdm),(Xx,Cc,YZdn),(Xx,Cc,YZdo),(Xx,Cc,YZdp),(Xx,Cc,YZdq),(Xx,Cc,YZdr),(Xx, Cc,YZds),(Xx,Cc,YZdt),(Xx,Cc,YZdu),(Xx,Cc,YZdv),(Xx,Cc,YZdw),(Xx,Cc,YZdx), (Xx,Cc,YZdy),(Xx,Cc,YZdz),(Xx,Cc,YZez),(Xx,Cc,YZeb),(Xx,Cc,YZec),(Xx,Cc,YZed), (Xx,Cc,YZee),(Xx,Cc,YZef),(Xx,Cc,YZeg),(Xx,Cc,YZeh),(Xx,Cc,YZei),(Xx,Cc, YZej),(Xx,Cc,YZek),(Xx,Cc,YZel),(Xx,Cc,YZem),(Xx,Cc,YZen),(Xx,Cc,YZeo),(Xx, Cc,YZep),(Xx,Cc,YZeq),(Xx,Cc,YZer),(Xx,Cc,YZes),(Xx,Cc,YZet),(Xx,Cc,YZeu),(XX, Cc,YZev),(Xx,Cc,YZew),(Xx,Cc,YZex),(Xx,Cc,YZey),(Xx,Cc,YZez),(Xx,Cc,YZfa), (Xx,Cc,YZfb),(Xx,Cc,YZfc),(Xx,Cc,YZfd),(Xx,Cc,YZfe),(Xx,Cc,YZff),(Xx,Cc,YZfg), (Xx,Cc,YZfh),(Xx,Cc,YZfi),(Xx,Cc,YZfj),(Xx,Cc,YZfk),(Xx,Cc,YZfl),(Xx,Cc,YZfm), (Xx,Cc,YZfn),(Xx,Cc,YZfo),(Xx,Cc,YZfp),(Xx,Cc,YZfq),(Xx,Cc,YZfr),(Xx,Cc,YZfs), (Xx,Cc,YZft),(Xx,Cc,YZfu),(Xx,Cc,YZfv),(Xx,Cc,YZfw),(Xx,Cc,YZfx),(Xx,Cc, YZfy),(Xx,Ce,YZfz),(Xx,Cc,YZga),(Xx,Ce,YZgb),(Xx,Ce,YZgc),(Xx,Ce,YZgd),(Xx, Cc,YZge),(Xx,Cc,YZgf),(Xx,Cc,YZgg),(Xx,Cc,YZgh),(Xx,Cc,YZgi),(Xx,Cc,YZgj),(Xx, Cc,YZgk),(Xx,Cc,YZgl),(Xx,Cc,YZgm),(Xx,Cc,YZgn),(Xx,Cc,YZgo),(Xx,Cc,YZgp), (Xx,Cc,YZgq),(Xx,Cc,YZgr),(Xx,Cc,YZgs),(Xx,Cc,YZgt),(Xx,Cd,YZa),(Xx,Cd,YZb), (Xx,Cd,YZc),(Xx,Cd,YZd),(Xx,Cd,YZe),(Xx,Cd,YZf),(Xx,Cd,YZg),(Xx,Cd,YZh), (Xx,Cd,YZi),(Xx,Cd,YZj),(Xx,Cd,YZk),(Xx,Cd,YZl),(Xx,Cd,YZm),(Xx,Cd,YZn),(Xx, Cd,YZo),(Xx,Cd,YZp),(Xx,Cd,YZq),(Xx,Cd,YZr),(Xx,Cd,YZs),(Xx,Cd,YZt),(Xx,Cd, YZu),(Xx,Cd,YZv),(Xx,Cd,YZw),(Xx,Cd,YZx),(Xx,Cd,YZy),(Xx,Cd,YZz),(Xx,Cd, YZaa),(Xx,Cd,YZab),(Xx,Cd,YZac),(Xx,Cd,YZad),(Xx,Cd,YZae),(Xx,Cd,YZaf),(Xx, Cd,YZag),(Xx,Cd,YZah),(Xx,Cd,YZai),(Xx,Cd,YZaj),(Xx,Cd,YZak),(Xx,Cd,YZal),(Xx, Cd,YZam),(Xx,Cd,YZan),(Xx,Cd,YZao),(Xx,Cd,YZap),(Xx,Cd,YZaq),(Xx,Cd,YZar), (Xx,Cd,YZas),(Xx,Cd,YZat),(Xx,Cd,YZau),(Xx,Cd,YZav),(Xx,Cd,YZaw),(Xx,Cd, YZax),(Xx,Cd,YZay),(Xx,Cd,YZaz),(Xx,Cd,YZba),(Xx,Cd,YZbb),(Xx,Cd,YZbc),(Xx, Cd,YZbd),(Xx,Cd,YZbe),(Xx,Cd,YZbf),(Xx,Cd,YZbg),(Xx,Cd,YZbh),(Xx,Cd,YZbi), (Xx,Cd,YZbj),(Xx,Cd,YZbk),(Xx,Cd,YZbl),(Xx,Cd,YZbm),(Xx,Cd,YZbn),(Xx,Cd,YZbo), (Xx,Cd,YZbp),(Xx,Cd,YZbq),(Xx,Cd,YZbr),(Xx,Cd,YZbs),(Xx,Cd,YZbt),(Xx,Cd, YZbu),(Xx,Cd,YZbv),(Xx,Cd,YZbw),(Xx,Cd,YZbx),(Xx,Cd,YZby),(Xx,Cd,YZbz),(Xx, Cd,YZca),(Xx,Cd,YZcb),(Xx,Cd,YZce),(Xx,Cd,YZcd),(Xx,Cd,YZce),(Xx,Cd,YZcf), (Xx,Cd,YZcg),(Xx,Cd,YZch),(Xx,Cd,YZci),(Xx,Cd,YZcj),(Xx,Cd,YZck),(Xx,Cd,YZcl), (Xx,Cd,YZcm),(Xx,Cd,YZcn),(Xx,Cd,YZco),(Xx,Cd,YZcp),(Xx,Cd,YZcq),(Xx, Cd,YZcr),(Xx,Cd,YZcs),(Xx,Cd,YZct),(Xx,Cd,YZcu),(Xx,Cd,YZcv),(Xx,Cd,YZcw),(Xx, Cd,YZcx),(Xx,Cd,YZcy),(Xx,Cd,YZcz),(Xx,Cd,YZda),(Xx,Cd,YZdb),(Xx,Cd,YZdc), (Xx,Cd,YZdd),(Xx,Cd,YZde),(Xx,Cd,YZdf),(Xx,Cd,YZdg),(Xx,Cd,YZdh),(Xx,Cd, YZdi),(Xx,Cd,YZdj),(Xx,Cd,YZdk),(Xx,Cd,YZdl),(Xx,Cd,YZdm),(Xx,Cd,YZdn),(Xx, Cd,YZdo),(Xx,Cd,YZdp),(Xx,Cd,YZdq),(Xx,Cd,YZdr),(Xx,Cd,YZds),(Xx,Cd,YZdt), (Xx,Cd,YZdu),(Xx,Cd,YZdv),(Xx,Cd,YZdw),(Xx,Cd,YZdx),(Xx,Cd,YZdy),(Xx,Cd, YZdz),(Xx,Cd,YZea),(Xx,Cd,YZeb),(Xx,Cd,YZec),(Xx,Cd,YZed),(Xx,Cd,YZee),(Xx, Cd,YZef),(Xx,Cd,YZeg),(Xx,Cd,YZeh),(Xx,Cd,YZei),(Xx,Cd,YZej),(Xx,Cd,YZek),(Xx, Cd,YZel),(Xx,Cd,YZem),(Xx,Cd,YZen),(Xx,Cd,YZeo),(Xx,Cd,YZep),(Xx,Cd,YZeq), (Xx,Cd,YZer),(Xx,Cd,YZes),(Xx,Cd,YZet),(Xx,Cd,YZeu),(Xx,Cd,YZev),(Xx,Cd, YZew),(Xx,Cd,YZex),(Xx,Cd,YZey),(Xx,Cd,YZez),(Xx,Cd,YZfa),(Xx,Cd,YZfb),(Xx, Cd,YZfc),(Xx,Cd,YZfd),(Xx,Cd,YZfe),(Xx,Cd,YZff),(Xx,Cd,YZfg),(Xx,Cd,YZfh),(Xx, Cd,YZfi),(Xx,Cd,YZfj),(Xx,Cd,YZfk),(Xx,Cd,YZfl),(Xx,Cd,YZfm),(Xx,Cd,YZfn),(Xx, Cd,YZfo),(Xx,Cd,YZfp),(Xx,Cd,YZfq),(Xx,Cd,YZfr),(Xx,Cd,YZfs),(Xx,Cd,YZft), (Xx,Cd,YZfu),(Xx,Cd,YZfv),(Xx,Cd,YZfw),(Xx,Cd,YZfx),(Xx,Cd,YZfy),(Xx,Cd,YZfz), (Xx,Cd,YZga),(Xx,Cd,YZgb),(Xx,Cd,YZgc),(Xx,Cd,YZgd),(Xx,Cd,YZge),(Xx,Cd, YZgf),(Xx,Cd,YZgg),(Xx,Cd,YZgh),(Xx,Cd,YZgi),(Xx,Cd,YZgj),(Xx,Cd,YZgk),(Xx, Cd,YZgl),(Xx,Cd,YZgm),(Xx,Cd,YZgn),(Xx,Cd,YZgo),(Xx,Cd,YZgp),(Xx,Cd,YZgq), (Xx,Cd,YZgr),(Xx,Cd,YZgs),(Xx,Cd,YZgt),(Xx,Ce,YZa),(Xx,Ce,YZb),(Xx,Ce,YZc), (Xx,Ce,YZd),(Xx,Ce,YZe),(Xx,Ce,YZf),(Xx,Ce,YZg),(Xx,Ce,YZh),(Xx,Ce,YZi),(Xx, Ce,YZj),(Xx,Ce,YZk),(Xx,Ce,YZl),(Xx,Ce,YZm),(Xx,Ce,YZn),(Xx,Ce,YZo),(Xx,Ce, YZp),(Xx,Ce,YZq),(Xx,Ce,YZr),(Xx,Ce,YZs),(Xx,Ce,YZt),(Xx,Ce,YZu),(Xx,Ce,YZv), (Xx,Ce,YZw),(Xx,Ce,YZx),(Xx,Ce,YZy),(Xx,Ce,YZz),(Xx,Ce,YZaa),(Xx,Ce,YZab), (Xx,Ce,YZac),(Xx,Ce,YZad),(Xx,CSe,YZae),(Xx,Ce,YZaf),(Xx,Ce,YZag),(Xx,Ce,YZah), (Xx,Ce,YZai),(Xx,Ce,YZaj),(Xx,Ce,YZak),(Xx,Ce,YZal),(Xx,Ce,YZam),(Xx,Ce, YZan),(Xx,Ce,YZao),(Xx,Ce,YZap),(Xx,Ce,YZaq),(Xx,Ce,YZar),(Xx,Ce,YZas),(Xx, Ce,YZat),(Xx,Ce,YZau),(Xx,Ce,YZav),(Xx,Ce,YZaw),(Xx,Ce,YZax),(Xx,Ce,YZay),(Xx, Ce,YZaz),(Xx,Ce,YZba),(Xx,Ce,YZbb),(Xx,Ce,YZbc),(Xx,Ce,YZbd),(Xx,Ce,YZbe), (Xx,Ce,YZbf),(Xx,Ce,YZbg),(Xx,Ce,YZbh),(Xx,Ce,YZbi),(Xx,Ce,YZbj),(Xx,Ce,YZbk), (Xx,Ce,YZbl),(Xx,Ce,YZbm),(Xx,Ce,YZbn),(Xx,Ce,YZbo),(Xx,Ce,YZbp),(Xx, Ce,YZbq),(Xx,Ce,YZbr),(Xx,Ce,YZbs),(Xx,Ce,YZbt),(Xx,Ce,YZbu),(Xx,Ce,YZbv),(Xx, Ce,YZbw),(Xx,Ce,YZbx),(Xx,Ce,YZby),(Xx,Ce,YZbz),(Xx,Ce,YZca),(Xx,Ce,YZcb), (Xx,Ce,YZcc),(Xx,Ce,YZcd),(Xx,Ce,YZce),(Xx,Ce,YZcf),(Xx,Ce,YZcg),(Xx,Ce, YZch),(Xx,Ce,YZci),(Xx,Ce,YZcj),(Xx,Ce,YZck),(Xx,Ce,YZcl),(Xx,Ce,YZcm),(Xx, Ce,YZcn),(Xx,Ce,YZco),(Xx,Ce,YZcp),(Xx,Ce,YZcq),(Xx,Ce,YZcr),(Xx,Ce,YZcs),(Xx, Ce,YZct),(Xx,Ce,YZcu),(Xx,Ce,YZcv),(Xx,Ce,YZcw),(Xx,Ce,YZcx),(Xx,Ce,YZcy), (Xx,Ce,YZcz),(Xx,Ce,YZda),(Xx,Ce,YZdb),(Xx,Ce,YZdc),(Xx,Ce,YZdd),(Xx,Ce, YZde),(Xx,Ce,YZdf),(Xx,Ce,YZdg),(Xx,Ce,YZdh),(Xx,Ce,YZdi),(Xx,Ce,YZdj),(Xx, Ce,YZdk),(Xx,Ce,YZdl),(Xx,Ce,YZdm),(Xx,Ce,YZdn),(Xx,Ce,YZdo),(Xx,Ce,YZdp), (Xx,Ce,YZdq),(Xx,Ce,YZdr),(Xx,Ce,YZds),(Xx,Ce,YZdt),(Xx,Ce,YZdu),(Xx,Ce,YZdv), (Xx,Ce,YZdw),(Xx,Ce,YZdx),(Xx,Ce,YZdy),(Xx,Ce,YZdz),(Xx,Ce,YZea),(Xx,Ce, YZeb),(Xx,Ce,YZec),(Xx,Ce,YZed),(Xx,Ce,YZee),(Xx,Ce,YZef),(Xx,Ce,YZeg),(Xx, Ce,YZeh),(Xx,Ce,YZei),(Xx,Ce,YZej),(Xx,Ce,YZek),(Xx,Ce,YZel),(Xx,Ce,YZem),(Xx, Ce,YZen),(Xx,Ce,YZeo),(XxCe,YZep),(Xx,Ce,YZeq),(Xx,Ce,YZer),(Xx,Ce,YZes), (Xx,Ce,YZet),(Xx,Ce,YZeu),(Xx,Ce,YZev),(Xx,Ce,YZew),(Xx,Ce,YZex),(Xx,Ce,YZey), (Xx,Ce,YZez),(Xx,Ce,YZfa),(Xx,Ce,YZfb),(Xx,Ce,YZfc),(Xx,Ce,YZfd),(Xx,Ce, YZfe),(Xx,Ce,YZff),(Xx,Ce,YZfg),(Xx,Ce,YZfh),(Xx,Ce,YZfi),(Xx,Ce,YZfj),(Xx,Ce, YZfk),(Xx,Ce,YZfl),(Xx,Ce,YZfm),(Xx,Ce,YZfn),(Xx,Ce,YZfo),(Xx,Ce,YZfp),(Xx,Ce, YZfq),(Xx,Ce,YZfr),(Xx,Ce,YZfs),(Xx,Ce,YZft),(Xx,Ce,YZfu),(Xx,Ce,YZfv),(Xx,Ce, YZfw),(Xx,Ce,YZfx),(Xx,Ce,YZfy),(Xx,Ce,YZfz),(Xx,Ce,YZga),(Xx,Ce,YZgb),(Xx, Ce,YZgc),(Xx,Ce,YZgd),(Xx,Ce,YZge),(Xx,Ce,YZgf),(Xx,Ce,YZgg),(Xx,Ce,YZgh), (Xx,Ce,YZgi),(Xx,Ce,YZgj),(Xx,Ce,YZgk),(Xx,Ce,YZgl),(Xx,Ce,YZgm),(Xx,Ce,YZgn), (Xx,Ce,YZgo),(Xx,Ce,YZgp),(Xx,Ce,YZgq),(Xx,Ce,YZgr),(Xx,Ce,YZgs),(Xx,Ce, YZgt),(Xx,Cf,YZa),(Xx,Cf,YZb),(Xx,Cf,YZc),(Xx,Cf,YZd),(Xx,Cf,YZe),(Xx,Cf,YZf), (Xx,Cf,YZg),(Xx,Cf,YZh),(Xx,Cf,YZi),(Xx,Cf,YZj),(Xx,Cf,YZk),(Xx,Cf,YZl),(Xx,Cf, YZm),(Xx,Cf,YZn),(Xx,Cf,YZo),(Xx,Cf,YZp),(Xx,Cf,YZq),(Xx,Cf,YZr),(Xx,Cf,YZs), (Xx,Cf,YZt),(Xx,Cf,YZu),(Xx,Cf,YZv),(Xx,Cf,YZw),(Xx,Cf,YZx),(Xx,Cf,YZy),(Xx, Cf,YZz),(Xx,Cf,YZaa),(Xx,Cf,YZab),(Xx,Cf,YZac),(Xx,Cf,YZad),(Xx,Cf,YZae),(Xx, Cf,YZaf),(Xx,Cf,YZag),(Xx,Cf,YZah),(Xx,Cf,YZai),(Xx,Cf,YZaj),(Xx,Cf,YZak),(Xx, Cf,YZal),(Xx,Cf,YZam),(Xx,Cf,YZan),(Xx,Cf,YZao),(Xx,Cf,YZap),(Xx,Cf,YZaq),(Xx, Cf,YZar),(Xx,Cf,YZas),(Xx,Cf,YZat),(Xx,Cf,YZau),(Xx,Cf,YZav),(Xx,Cf,YZaw), (Xx,Cf,YZax),(Xx,Cf,YZay),(Xx,Cf,YZaz),(Xx,Cf,YZba),(Xx,Cf,YZbb),(Xx,Cf,YZbc), (Xx,Cf,YZbd),(Xx,Cf,YZbe),(Xx,Cf,YZbf),(Xx,Cf,YZbg),(Xx,Cf,YZbh),(Xx,Cf,YZbi), (Xx,Cf,YZbj),(Xx,Cf,YZbk),(Xx,Cf,YZbl),(Xx,Cf,YZbm),(Xx,Cf,YZbn),(Xx,Cf,YZbo), (Xx,Cf,YZbp),(Xx,Cf,YZbq),(Xx,Cf,YZbr),(Xx,Cf,YZbs),(Xx,Cf,YZbt),(Xx,Cf, YZbu),(Xx,Cf,YZbv),(Xx,Cf,YZbw),(Xx,Cf,YZbx),(Xx,Cf,YZby),(Xx,Cf,YZbz),(Xx, Cf,YZca),(Xx,Cf,YZcb),(Xx,Cf,YZcc),(Xx,Cf,YZcd),(Xx,Cf,YZce),(Xx,Cf,YZcf),(Xx, Cf,YZcg),(Xx,Cf,YZch),(Xx,Cf,YZci),(Xx,Cf,YZcj),(Xx,Cf,YZck),(Xx,Cf,YZcl),(Xx, Cf,YZcm),(Xx,Cf,YZcn),(Xx,Cf,YZco),(Xx,Cf,YZcp),(Xx,Cf,YZcq),(Xx,Cf,YZcr),(Xx, Cf,YZcs),(Xx,Cf,YZct),(Xx,Cf,YZcu),(Xx,Cf,YZcv),(Xx,Cf,YZcw),(Xx,Cf,YZcx), (Xx,Cf,YZcy),(Xx,Cf,YZcz),(Xx,Cf,YZda),(Xx,Cf,YZdb),(Xx,Cf,YZdc),(Xx,Cf,YZdd), (Xx,Cf,YZde),(Xx,Cf,YZdf),(Xx,Cf,YZdg),(Xx,Cf,YZdh),(Xx,Cf,YZdi),(Xx,Cf,YZdj), (Xx,Cf,YZdk),(Xx,Cf,YZdl),(Xx,Cf,YZdm),(Xx,Cf,YZdn),(Xx,Cf,YZdo),(Xx,Cf, YZdp),(Xx,Cf,YZdq),(Xx,Cf,YZdr),(Xx,Cf,YZds),(Xx,Cf,YZdt),(Xx,Cf,YZdu),(Xx,Cf, YZdv),(Xx,Cf,YZdw),(Xx,Cf,YZdx),(Xx,Cf,YZdy),(Xx,Cf,YZdz),(Xx,Cf,YZea),(Xx, Cf,YZeb),(Xx,Cf,YZec),(Xx,Cf,YZed),(Xx,Cf,YZee),(Xx,Cf,YZef),(Xx,Cf,YZeg),(Xx, Cf,YZeh),(Xx,Cf,YZei),(Xx,Cf,YZej),(Xx,Cf,YZek),(Xx,Cf,YZel),(Xx,Cf,YZem),(Xx, Cf,YZen),(Xx,Cf,YZeo),(Xx,Cf,YZep),(Xx,Cf,YZeq),(Xx,Cf,YZer),(Xx,Cf,YZes),(Xx, Cf,YZet),(Xx,Cf,YZeu),(Xx,Cf,YZev),(Xx,Cf,YZew),(Xx,Cf,YZex),(Xx,Cf,YZey), (Xx,Cf,YZez),(Xx,Cf,YZfa),(Xx,Cf,YZfb),(Xx,Cf,YZfc),(Xx,Cf,YZfd),(Xx,Cf,YZfe), (Xx,Cf,YZff),(Xx,Cf,YZfg),(Xx,Cf,YZfh),(Xx,Cf,YZfi),(Xx,Cf,YZfj),(Xx,Cf,YZfk),(Xx, Cf,YZfl),(Xx,Cf,YZfm),(Xx,Cf,YZfn),(Xx,Cf,YZfo),(Xx,Cf,YZfp),(Xx,Cf,YZfq),(Xx, Cf,YZfr),(Xx,Cf,YZfs),(Xx,cf,YZft),(Xx,Cf,YZfu),(Xx,cf,YZfv),(Xx,Cf,YZfw),(Xx, Cf,YZfx),(Xx,Cf,YZfy),(Xx,Cf,YZfz),(Xx,Cf,YZga),(Xx,Cf,YZgb),(Xx,Cf,YZgc), (Xx,Cf,YZgd),(YXx,Cf,YZge),(Xx,Cf,YZgf),(Xx,Cf,YZgg),(Xx,Cf,YZgh),(Xx,Cf,YZgi), (Xx,Cf,YZgj),(Xx,Cf,YZgk),(Xx,Cf,YZgl),(Xx,Cf YZgm),(Xx,Cf,YZgn),(Xx,Cf,YZgo), (Xx,Cf,YZgp),(Xx,Cf,YZgq),(Xvx,Cf,YZgr),(Xxc,Cf,YZgs),(Xx,Cf,YZgt), (Xy,Ca,YZa),(Xy,Ca,YZb),(Xy,Ca,YZc),(Xy,Ca,YZd),(Xy,Ca,YZe),(Xy,Ca,YZf),(Xy, Ca,YZg),(Xy,Ca,YZh),(Xy,Ca,YZi),(Xy,Ca,YZj),(Xy,Ca,YZk),(Xy,Ca,YZl),(Xy,Ca,YZm), (Xy,Ca,YZn),(Xy,Ca,YZo),(Xy,Ca,YZp),(Xy,Ca,YZq),(Xy,Ca,YZr),(Xy,Ca,YZs), (Xy,Ca,YZt),(Xy,Ca,YZu),(Xy,Ca,YZv),(Xy,Ca,YZw),(Xy,Ca,YZx),(Xy,Ca,YZy),(Xy, Ca,YZz),(Xy,Ca,YZaa),(Xy,Ca,YZab),(Xy,Ca,YZac),(Xy,Ca,YZad),(Xy,Ca,YZae),(Xy, Ca,YZaf),(Xy,Ca,YZag),(Xy,Ca,YZah),(Xy,Ca,YZai),(Xy,Ca,YZaj),(Xy,Ca,YZak), (Xy,Ca,YZal),(Xy,Ca,YZam),(Xy,Ca,YZan),(Xy,Ca,YZao),(Xy,Ca,YZap),(Xy,Ca,YZaq), (Xy,Ca,YZar),(Xy,Ca,YZas),(Xy,Ca,YZat),(Xy,Ca,YZau),(Xy,Ca,YZav),(Xy,Ca, YZaw),(Xy,Ca,YZax),(Xy,Ca,YZay),(Xy,Ca,YZaz),(Xy,Ca,YZba),(Xy,Ca,YZbb),(Xy, Ca,YZbc),(Xy,Ca,YZbd),(Xy,Ca,YZbe),(Xy,Ca,YZbf),(Xy,Ca,YZbg),(Xy,Ca,YZbh),(Xy, Ca,YZbi),(Xy,Ca,YZbj),(Xy,Ca,YZbk),(Xy,Ca,YZbl),(Xy,Ca,YZbm),(Xy,Ca,YZbn), (Xy,Ca,YZbo),(Xy,Ca,YZbp),(Xy,Ca,YZbq),(Xy,Ca,YZbr),(Xy,Ca,YZbs),(Xy,Ca, YZbt),(Xy,Ca,YZbu),(Xy,Ca,YZbv),(Xy,Ca,YZbw),(Xy,Ca,YZbx),(Xy,Ca,YZby),(Xy, Ca,YZbz),(Xy,Ca,YZca),(Xy,Ca,YZcb),(Xy,Ca,YZcc),(Xy,Ca,YZcd),(Xy,Ca,YZce),(Xy, Ca,YZcf),(Xy,Ca,YZcg),(Xy,Ca,YZch),(Xy,Ca,YZci),(Xy,Ca,YZcj),(Xy,Ca,YZck), (Xy,Ca,YZcl),(Xy,Ca,YZcm),(Xy,Ca,YZcn),(Xy,Ca,YZco),(Xy,Ca,YZcp),(Xy,Ca,YZcq), (Xy,Ca,YZcr),(Xy,Ca,YZcs),(Xy,Ca,YZct),(Xy,Ca,YZcu),(Xy,Ca,YZcv),(Xy,Ca, YZcw),(Xy,Ca,YZcx),(Xy,Ca,YZcy),(Xy,Ca,YZcz),(Xy,Ca,YZda),(Xy,Ca,YZdb),(Xy, Ca,YZdc),(Xy,Ca,YZdd),(Xy,Ca,YZde),(Xy,Ca,YZdf),(Xy,Ca,YZdg),(Xy,Ca,YZdh),(Xy, Ca,YZdi),(Xy,Ca,YZdj),(Xy,Ca,YZdk),(Xy,Ca,YZdl),(Xy,Ca,YZdm),(Xy,Ca,YZdn), (Xy,Ca,YZdo),(Xy,Ca,YZdp),(Xy,Ca,YZdq),(Xy,Ca,YZdr),(Xy,Ca,YZds),(Xy,Ca, YZdt),(Xy,Ca,YZdu),(Xy,Ca,YZdv),(Xy,Ca,YZdw),(Xy,Ca,YZdx),(Xy,Ca,YZdy),(Xy, Ca,YZdz),(Xy,Ca,YZea),(Xy,Ca,YZeb),(Xy,Ca,YZec),(Xy,Ca,YZed),(Xy,Ca,YZee),(Xy, Ca,YZef),(Xy,Ca,YZeg),(Xy,Ca,YZeh),(Xy,Ca,YZei),(Xy,Ca,YZej),(Xy,Ca,YZek), (Xy,Ca,YZel),(Xy,Ca,YZem),(Xy,Ca,YZen),(Xy,Ca,YZeo),(Xy,Ca,YZep),(Xy,Ca,YZeq), (Xy,Ca,YZer),(Xy,Ca,YZes),(Xy,Ca,YZet),(Xy,Ca,YZeu),(Xy,Ca,YZev),(Xy,Ca, YZew),(Xy,Ca,YZex),(Xy,Ca,YZey),(Xy,Ca,YZez),(Xy,Ca,YZfa),(Xy,Ca,YZfb),(Xy, Ca,YZfc),(Xy,Ca,YZfd),(Xy,Ca,YZfe),(Xy,Ca,YZff),(Xy,Ca,YZfg),(Xy,Ca,YZfh),(Xy, Ca,YZfi),(Xy,Ca,YZfj),(Xy,Ca,YZfk),(Xy,Ca,YZfl),(Xy,Ca,YZfm),(Xy,Ca,YZfn),(Xy, Ca,YZfo),(Xy,Ca,YZfp),(Xy,Ca,YZfq),(Xy,Ca,YZfr),(Xy,Ca,YZfs),(Xy,Ca,YZft),(Xy, Ca,YZfu),(Xy,Ca,YZfv),(Xy,Ca,YZfw),(Xy,Ca,YZfx),(Xy,Ca,YZfy),(Xy,Ca,YZfz),(Xy, Ca,YZga),(Xy,Ca,YZgb),(Xy,Ca,YZgc),(Xy,Ca,YZgd),(Xy,Ca,YZge),(Xy,Ca,YZgf), (Xy,Ca,YZgg),(Xy,Ca,YZgh),(Xy,Ca,YZgi),(Xy,Ca,YZgj),(Xy,Ca,YZgk),(Xy,Ca,YZgl), (Xy,Ca,YZgm),(Xy,Ca,YZgn),(Xy,Ca,YZgo),(Xy,Ca,YZgp),(Xy,Ca,YZgq),(Xy, Ca,YZgr),(Xy,Ca,YZgs),(Xy,Ca,YZgt),(Xy,Cb,YZa),(Xy,Cb,YZb),(Xy,Cb,YZc),(Xy, Cb,YZd),(Xy,Cb,YZe),(Xy,Cb,YZf),(Xy,Cb,YZg),(Xy,Cb,YZh),(Xy,Cb,YZi),(Xy,Cb, YZj),(Xy,Cb,YZk),(Xy,Cb,YZl),(Xy,Cb,YZm),(Xy,Cb,YZn),(Xy,Cb,YZo),(Xy,Cb,YZp), (Xy,Cb,YZq),(Xy,Cb,YZr),(Xy,Cb,YZs),(Xy,Cb,YZt),(Xy,Cb,YZu),(Xy,Cb,YZv),(Xy, Cb,YZw),(Xy,Cb,YZx),(Xy,Cb,YZy),(Xy,Cb,YZz),(Xy,Cb,YZaa),(Xy,Cb,YZab),(Xy, Cb,YZac),(Xy,Cb,YZad),(Xy,Cb,YZae),(Xy,Cb,YZaf),(Xy,Cb,YZag),(Xy,Cb,YZah), (Xy,Cb,YZai),(Xy,Cb,YZaj),(Xy,Cb,YZak),(Xy,Cb,YZal),(Xy,Cb,YZam),(Xy,Cb, YZan),(Xy,Cb,YZao),(Xy,Cb,YZap),(Xy,Cb,YZaq),(Xy,Cb,YZar),(Xy,Cb,YZas),(Xy, Cb,YZat),(Xy,Cb,YZau),(Xy,Cb,YZav),(Xy,Cb,YZaw),(Xy,Cb,YZax),(Xy,Cb,YZay), (Xy,Cb,YZaz),(Xy,Cb,YZba),(Xy,Cb,YZbb),(Xy,Cb,YZbc),(Xy,Cb,YZbd),(Xy,Cb,YZbe), (Xy,Cb,YZbf),(Xy,Cb,YZbg),(Xy,Cb,YZbh),(Xy,Cb,YZbi),(Xy,Cb,YZbj),(Xy,Cb, YZbk),(Xy,Cb,YZbl),(Xy,Cb,YZbm),(Xy,Cb,YZbn),(Xy,Cb,YZbo),(Xy,Cb,YZbp),(Xy, Cb,YZbq),(Xy,Cb,YZbr),(Xy,Cb,YZbs),(Xy,Cb,YZbt),(Xy,Cb,YZbu),(Xy,Cb,YZbv), (Xy,Cb,YZbw),(Xy,Cb,YZbx),(Xy,Cb,YZby),(Xy,Cb,YZbz),(Xy,Cb,YZca),(Xy,Cb, YZcb),(Xy,Cb,YZcc),(Xy,Cb,YZcd),(Xy,Cb,YZce),(Xy,Cb,YZcf),(Xy,Cb,YZcg),(Xy, Cb,YZch),(Xy,Cb,YZci),(Xy,Cb,YZcj),(Xy,Cb,YZck),(Xy,Cb,YZcl),(Xy,Cb,YZcm),(Xy, Cb,YZcn),(Xy,Cb,YZco),(Xy,Cb,YZcp),(Xy,Cb,YZcq),(Xy,Cb,YZcr),(Xy,Cb,YZcs), (Xy,Cb,YZct),(Xy,Cb,YZcu),(Xy,Cb,YZcv),(Xy,Cb,YZcw),(Xy,Cb,YZcx),(Xy,Cb, YZcy),(Xy,Cb,YZcz),(Xy,Cb,YZda),(Xy,Cb,YZdb),(Xy,Cb,YZdc),(Xy,Cb,YZdd),(Xy, Cb,YZde),(Xy,Cb,YZdf),(Xy,Cb,YZdg),(Xy,Cb,YZdh),(Xy,Cb,YZdi),(Xy,Cb,YZdj),(Xy, Cb,YZdk),(Xy,Cb,YZdl),(Xy,Cb,YZdm),(Xy,Cb,YZdn),(Xy,Cb,YZdo),(Xy,Cb,YZdp), (Xy,Cb,YZdq),(Xy,Cb,YZdr),(Xy,Cb,YZds),(Xy,Cb,YZdt),(Xy,Cb,YZdu),(Xy,Cb, YZdv),(Xy,Cb,YZdw),(Xy,Cb,YZdx),(Xy,Cb,YZdy),(Xy,Cb,YZdz),(Xy,Cb,YZea),(Xy, Cb,YZeb),(Xy,Cb,YZec),(Xy,Cb,YZed),(Xy,Cb,YZee),(Xy,Cb,YZef),(Xy,Cb,YZeg), (Xy,Cb,YZeh),(Xy,Cb,YZei),(Xy,Cb,YZej),(Xy,Cb,YZek),(Xy,Cb,YZel),(Xy,Cb,YZem), (Xy,Cb,YZen),(Xy,Cb,YZeo),(Xy,Cb,YZep),(Xy,Cb,YZeq),(Xy,Cb,YZer),(Xy,Cb, YZes),(Xy,Cb,YZet),(Xy,Cb,YZeu),(Xy,Cb,YZev),(Xy,Cb,YZew),(Xy,Cb,YZex),(Xy, Cb,YZey),(Xy,Cb,YZez),(Xy,Cb,YZfa),(Xy,Cb,YZfb),(Xy,Cb,YZfc),(Xy,Cb,YZfd),(Xy, Cb,YZfe),(Xy,Cb,YZff),(Xy,Cb,YZfg),(Xy,Cb,YZfh),(Xy,Cb,YZfi),(Xy,Cb,YZfj), (Xy,Cb,YZfk),(Xy,Cb,YZfl),(Xy,Cb,YZfm),(Xy,Cb,YZfn),(Xy,Cb,YZfo),(Xy,Cb,YZfp), (Xy,Cb,YZfq),(Xy,Cb,YZfr),(Xy,Cb,YZfs),(Xy,Cb,YZft),(Xy,Cb,YZfu),(Xy,Cb,YZfv), (Xy,Cb,YZfw),(Xy,Cb,YZfx),(Xy,Cb,YZfy),(Xy,Cb,YZfz),(Xy,Cb,YZga),(Xy,Cb, YZgb),(Xy,Cb,YZgc),(Xy,Cb,YZgd),(Xy,Cb,YZge),(Xy,Cb,YZgf),(Xy,Cb,YZgg),(Xy, Cb,YZgh),(Xy,Cb,YZgi),(Xy,Cb,YZgj),(Xy,Cb,YZgk),(Xy,Cb,YZgl),(Xy,Cb,YZgm), (Xy,Cb,YZgn),(Xy,Cb,YZgo),(Xy,Cb,YZgp),(Xy,Cb,YZgq),(Xy,Cb,YZgr),(Xy,Cb,YZgs), (Xy,Cb,YZgt),(Xy,Cc,YZa),(Xy,Cc,YZb),(Xy,Cc,YZc),(Xy,Cc,YZd),(Xy,Cc,YZe), (Xy,Cc,YZf),(Xy,Cc,YZg),(Xy,Cc,YZh),(Xy,Cc,YZi),(Xy,Cc,YZj),(Xy,Cc,YZk),(Xy, Cc,YZl),(Xy,Cc,YZm),(Xy,Cc,YZn),(Xy,Cc,YZo),(Xy,Cc,YZp),(Xy,Cc,YZq),(Xy,Cc, YZr),(Xy,Cc,YZs),(Xy,Cc,YZt),(Xy,Cc,YZu),(Xy,Cc,YZv),(Xy,Cc,YZw),(Xy,Cc,YZx), (Xy,Cc,YZy),(Xy,Cc,YZz),(Xy,Cc,YZaa),(Xy,Cc,YZab),(Xy,Cc,YZac),(Xy,Cc,YZad), (Xy,Cc,YZae),(Xy,Cc,YZaf),(Xy,Cc,YZag),(Xy,Cc,YZah),(Xy,Cc,YZai),(Xy,Cc, YZaj),(Xy,Cc,YZak),(Xy,Cc,YZal),(Xy,Cc,YZam),(Xy,Cc,YZan),(Xy,Cc,YZao),(Xy, Cc,YZap),(Xy,Cc,YZaq),(Xy,Cc,YZar),(Xy,Cc,YZas),(Xy,Cc,YZat),(Xy,Cc,YZau),(Xy, Cc,YZav),(Xy,Cc,YZaw),(Xy,Cc,YZax),(Xy,Cc,YZay),(Xy,Cc,YZaz),(Xy,Cc,YZba), (Xy,Cc,YZbb),(Xy,Cc,YZbc),(Xy,Cc,YZbd),(Xy,Cc,YZbe),(Xy,Cc,YZbf),(Xy,Cc,YZbg), (Xy,Cc,YZbh),(Xy,Cc,YZbi),(Xy,Cc,YZbj),(Xy,Cc,YZbk),(Xy,Cc,YZbl),(Xy,Cc, YZbm),(Xy,Cc,YZbn),(Xy,Cc,YZbo),(Xy,Cc,YZbp),(Xy,Cc,YZbq),(Xy,Cc,YZbr),(Xy, Cc,YZbs),(Xy,Cc,YZbt),(Xy,Cc,YZbu),(Xy,Cc,YZbv),(Xy,Cc,YZbw),(Xy,Cc,YZbx), (Xy,Cc,YZby),(Xy,Cc,YZbz),(Xy,Cc,YZca),(Xy,Cc,YZcb),(Xy,Cc,YZcc),(Xy,Cc,YZcd), (Xy,Cc,YZce),(Xy,Cc,YZcf),(Xy,Cc,YZcg),(Xy,Cc,YZch),(Xy,Cc,YZci),(Xy,Cc, YZcj),(Xy,Cc,YZck),(Xy,Cc,YZcl),(Xy,Cc,YZcm),(Xy,Cc,YZcn),(Xy,Cc,YZco),(Xy, Cc,YZcp),(Xy,Cc,YZcq),(Xy,Cc,YZcr),(Xy,Cc,YZcs),(Xy,Cc,YZct),(Xy,Cc,YZcu),(Xy, Cc,YZcv),(Xy,Cc,YZcw),(Xy,Cc,YZcx),(Xy,Cc,YZcy),(Xy,Cc,YZcz),(Xy,Cc,YZda), (Xy,Cc,YZdb),(Xy,Cc,YZdc),(Xy,Cc,YZdd),(Xy,Cc,YZde),(Xy,Cc,YZdf),(Xy,Cc,YZdg), (Xy,Cc,YZdh),(Xy,Cc,YZdi),(Xy,Cc,YZdj),(Xy,Cc,YZdk),(Xy,Cc,YZdl),(Xy,Cc, YZdm),(Xy,Cc,YZdn),(Xy,Cc,YZdo),(Xy,Cc,YZdp),(Xy,Cc,YZdq),(Xy,Cc,YZdr),(Xy, Cc,YZds),(Xy,Cc,YZdt),(Xy,Cc,YZdu),(Xy,Cc,YZdv),(Xy,Cc,YZdw),(Xy,Cc,YZdx), (Xy,Cc,YZdy),(Xy,Cc,YZdz),(Xy,Cc,YZea),(Xy,Cc,YZeb),(Xy,Cc,YZec),(Xy,Cc,YZed), (Xy,Cc,YZee),(Xy,Cc,YZef),(Xy,Cc,YZeg),(Xy,Cc,YZeh),(Xy,Cc,YZei),(Xy,Cc, YZej),(Xy,Cc,YZek),(Xy,Cc,YZel),(Xy,Cc,YZem),(Xy,Cc,YZen),(Xy,Cc,YZeo),(Xy, Cc,YZep),(Xy,Cc,YZeq),(Xy,Cc,YZer),(Xy,Cc,YZes),(Xy,Cc,YZet),(Xy,Cc,YZeu),(Xy, Cc,YZev),(Xy,Cc,YZew),(Xy,Cc,YZex),(Xy,Cc,YZey),(Xy,Cc,YZez),(Xy,Cc,YZfa), (Xy,Cc,YZfb),(Xy,Cc,YZfc),(Xy,Cc,YZfd),(Xy,Cc,YZfe),(Xy,Cc,YZff),(Xy,Cc,YZfg), (Xy,Cc,YZfh),(Xy,Cc,YZfi),(Xy,Cc,YZfj),(Xy,Cc,YZfk),(Xy,Cc,YZfl),(Xy,Cc,YZfm), (Xy,Cc,YZfn),(Xy,Cc,YZfo),(Xy,Cc,YZfp),(Xy,Cc,YZfq),(Xy,Cc,YZfr),(Xy,Cc,YZfs), (Xy,Cc,YZft),(Xy,Cc,YZfu),(Xy,Cc,YZfv),(Xy,Cc,YZfw),(Xy,Cc,YZfx),(Xy,Cc, YZfy),(Xy,Cc,YZfz),(Xy,Cc,YZga),(Xy,Cc,YZgb),(Xy,Cc,YZgc),(Xy,Cc,YZgd),(Xy, Cc,YZge),(Xy,Cc,YZgf),(Xy,Cc,YZgg),(Xy,Cc,YZgh),(Xy,Cc,YZgi),(Xy,Cc,YZgj),(Xy, Cc,YZgk),(Xy,Cc,YZgl),(Xy,Cc,YZgm),(Xy,Cc,YZgn),(Xy,Cc,YZgo),(Xy,Cc,YZgp), (Xy,Cc,YZgq),(Xy,Cc,YZgr),(Xy,Cc,YZgs),(Xy,Cc,YZgt),(Xy,Cd,YZa),(Xy,Cd,YZb), (Xy,Cd,YZc),(Xy,Cd,YZd),(Xy,Cd,YZe),(Xy,Cd,YZf),(Xy,Cd,YZg),(Xy,Cd,YZh), (Xy,Cd,YZi),(Xy,Cd,YZj),(Xy,Cd,YZk),(Xy,Cd,YZl),(Xy,Cd,YZm),(Xy,Cd,YZn),(Xy, Cd,YZo),(Xy,Cd,YZp),(Xy,Cd,YZq),(Xy,Cd,YZr),(Xy,Cd,YZs),(Xy,Cd,YZt),(Xy,Cd, YZu),(Xy,Cd,YZv),(Xy,Cd,YZw),(Xy,Cd,YZx),(Xy,Cd,YZy),(Xy,Cd,YZz),(Xy,Cd, YZaa),(Xy,Cd,YZab),(Xy,Cd,YZac),(Xy,Cd,YZad),(Xy,Cd,YZae),(Xy,Cd,YZaf),(Xy, Cd,YZag),(Xy,Cd,YZah),(Xy,Cd,YZai),(Xy,Cd,YZaj),(Xy,Cd,YZak),(Xy,Cd,YZal),(Xy, Cd,YZam),(Xy,Cd,YZan),(Xy,Cd,YZao),(Xy,Cd,YZap),(Xy,Cd,YZaq),(Xy,Cd,YZar), (Xy,Cd,YZas),(Xy,Cd,YZat),(Xy,Cd,YZau),(Xy,Cd,YZav),(Xy,Cd,YZaw),(Xy,Cd, YZax),(Xy,Cd,YZay),(Xy,Cd,YZaz),(Xy,Cd,YZba),(Xy,Cd,YZbb),(Xy,Cd,YZbc),(Xy, Cd,YZbd),(Xy,Cd,YZbe),(Xy,Cd,YZbf),(Xy,Cd,YZbg),(Xy,Cd,YZbh),(Xy,Cd,YZbi), (Xy,Cd,YZbj),(Xy,Cd,YZbk),(Xy,Cd,YZbl),(Xy,Cd,YZbm),(Xy,Cd,YZbn),(Xy,Cd,YZbo), (Xy,Cd,YZbp),(Xy,Cd,YZbq),(Xy,Cd,YZbr),(Xy,Cd,YZbs),(Xy,Cd,YZbt),(Xy,Cd, YZbu),(Xy,Cd,YZbv),(Xy,Cd,YZbw),(Xy,Cd,YZbx),(Xy,Cd,YZby),(Xy,Cd,YZbz),(Xy, Cd,YZca),(Xy,Cd,YZcb),(Xy,Cd,YZcc),(Xy,Cd,YZcd),(Xy,Cd,YZce),(Xy,Cd,YZcf), (Xy,Cd,YZcg),(Xy,Cd,YZch),(Xy,Cd,YZci),(Xy,Cd,YZcj),(Xy,Cd,YZck),(Xy,Cd,YZcl), (Xy,Cd,YZcm),(Xy,Cd,YZcn),(Xy,Cd,YZco),(Xy,Cd,YZcp),(Xy,Cd,YZcq),(Xy, Cd,YZcr),(Xy,Cd,YZcs),(Xy,Cd,YZct),(Xy,Cd,YZcu),(Xy,Cd,YZcv),(Xy,Cd,YZcw),(Xy, Cd,YZcx),(Xy,Cd,YZcy),(Xy,Cd,YZcz),(Xy,Cd,YZda),(Xy,Cd,YZdb),(Xy,Cd,YZdc), (Xy,Cd,YZdd),(Xy,Cd,YZde),(Xy,Cd,YZdf),(Xy,Cd,YZdg),(Xy,Cd,YZdh),(Xy,Cd, YZdi),(Xy,Cd,YZdj),(Xy,Cd,YZdk),(Xy,Cd,YZdl),(Xy,Cd,YZdm),(Xy,Cd,YZdn),(Xy, Cd,YZdo),(Xy,Cd,YZdp),(Xy,Cd,YZdq),(Xy,Cd,YZdr),(Xy,Cd,YZds),(Xy,Cd,YZdt), (Xy,Cd,YZdu),(Xy,Cd,YZdv),(Xy,Cd,YZdw),(Xy,Cd,YZdx),(Xy,Cd,YZdy),(Xy,Cd, YZdz),(Xy,Cd,YZea),(Xy,Cd,YZeb),(Xy,Cd,YZec),(Xy,Cd,YZed),(Xy,Cd,YZee),(Xy, Cd,YZef),(Xy,Cd,YZeg),(Xy,Cd,YZeh),(Xy,Cd,YZei),(Xy,Cd,YZej),(Xy,Cd,YZek),(Xy, Cd,YZel),(Xy,Cd,YZem),(Xy,Cd,YZen),(Xy,Cd,YZeo),(Xy,Cd,YZep),(Xy,Cd,YZeq), (Xy,Cd,YZer),(Xy,Cd,YZes),(Xy,Cd,YZet),(Xy,Cd,YZeu),(Xy,Cd,YZev),(Xy,Cd, YZew),(Xy,Cd,YZex),(Xy,Cd,YZey),(Xy,Cd,YZez),(Xy,Cd,YZfa),(Xy,Cd,YZfb),(Xy, Cd,YZfc),(Xy,Cd,YZfd),(Xy,Cd,YZfe),(Xy,Cd,YZff),(Xy,Cd,YZfg),(Xy,Cd,YZfh),(Xy, Cd,YZfi),(Xy,Cd,YZfj),(Xy,Cd,YZfk),(Xy,Cd,YZfl),(Xy,Cd,YZfm),(Xy,Cd,YZfn),(Xy, Cd,YZfo),(Xy,Cd,YZfp),(Xy,Cd,YZfq),(Xy,Cd,YZfr),(Xy,Cd,YZfs),(Xy,Cd,YZft), (Xy,Cd,YZfu),(Xy,Cd,YZfv),(Xy,Cd,YZfw),(Xy,Cd,YZfx),(Xy,Cd,YZfy),(Xy,Cd,YZfz), (Xy,Cd,YZga),(Xy,Cd,YZgb),(Xy,Cd,YZgc),(Xy,Cd,YZgd),(Xy,Cd,YZge),(Xy,Cd, YZgf),(Xy,Cd,YZgg),(Xy,Cd,YZgh),(Xy,Cd,YZgi),(Xy,Cd,YZgj),(Xy,Cd,YZgk),(Xy, Cd,YZgl),(Xy,Cd,YZgm),(Xy,Cd,YZgn),(Xy,Cd,YZgo),(Xy,Cd,YZgp),(Xy,Cd,YZgq), (Xy,Cd,YZgr),(Xy,Cd,YZgs),(Xy,Cd,YZgt),(Xy,Ce,YZa),(Xy,Ce,YZb),(Xy,Ce,YZc), (Xy,Ce,YZd),(Xy,Ce,YZe),(Xy,Ce,YZf),(Xy,Ce,YZg),(Xy,Ce,YZh),(Xy,Ce,YZi),(Xy, Ce,YZj),(Xy,Ce,YZk),(Xy,Ce,YZl),(Xy,Ce,YZm),(Xy,Ce,YZn),(Xy,Ce,YZo),(Xy,Ce, YZp),(Xy,Ce,YZq),(Xy,Ce,YZr),(Xy,Ce,YZs),(Xy,Ce,YZt),(Xy,Ce,YZu),(Xy,Ce,YZv), (Xy,Ce,YZw),(Xy,Ce,YZx),(Xy,Ce,YZy),(Xy,Ce,YZz),(Xy,Ce,YZaa),(Xy,Ce,YZab), (Xy,Ce,YZac),(Xy,Ce,YZad),(Xy,Ce,YZae),(Xy,Ce,YZaf),(Xy,Ce,YZag),(Xy,Ce,YZah), (Xy,Ce,YZai),(Xy,Ce,YZaj),(Xy,Ce,YZak),(Xy,Ce,YZal),(Xy,Ce,YZam),(Xy,Ce, YZan),(Xy,Ce,YZao),(Xy,Ce,YZap),(Xy,Ce,YZaq),(Xy,Ce,YZar),(Xy,Ce,YZas),(Xy, Ce,YZat),(Xy,Ce,YZau),(Xy,Ce,YZav),(Xy,Ce,YZaw),(Xy,Ce,YZax),(Xy,Ce,YZay),(Xy, Ce,YZaz),(Xy,Ce,YZba),(Xy,Ce,YZbb),(Xy,Ce,YZbc),(Xy,Ce,YZbd),(Xy,Ce,YZbe), (Xy,Ce,YZbf),(Xy,Ce,YZbg),(Xy,Ce,YZbh),(Xy,Ce,YZbi),(Xy,Ce,YZbj),(Xy,Ce,YZbk), (Xy,Ce,YZbl),(Xy,Ce,YZbm),(Xy,Ce,YZbn),(Xy,Ce,YZbo),(Xy,Ce,YZbp),(Xy, Ce,YZbq),(Xy,Ce,YZbr),(Xy,Ce,YZbs),(Xy,Ce,YZbt),(Xy,Ce,YZbu),(Xy,Ce,YZbv),(Xy, Ce,YZbw),(Xy,Ce,YZbx),(Xy,Ce,YZby),(Xy,Ce,YZbz),(Xy,Ce,YZca),(Xy,Ce,YZcb), (Xy,Ce,YZcc),(Xy,Ce,YZcd),(Xy,Ce,YZce),(Xy,Ce,YZcf),(Xy,Ce,YZcg),(Xy,Ce, YZch),(Xy,Ce,YZci),(Xy,Ce,YZcj),(Xy,Ce,YZck),(Xy,Ce,YZcl),(Xy,Ce,YZcm),(Xy, Ce,YZcn),(Xy,Ce,YZco),(Xy,Ce,YZcp),(Xy,Ce,YZcq),(Xy,Ce,YZcr),(Xy,Ce,YZcs),(Xy, Ce,YZct),(Xy,Ce,YZcu),(Xy,Ce,YZcv),(Xy,Ce,YZcw),(Xy,Ce,YZcx),(Xy,Ce,YZcy), (Xy,Ce,YZcz),(Xy,Ce,YZda),(Xy,Ce,YZdb),(Xy,Ce,YZdc),(Xy,Ce,YZdd),(Xy,Ce, YZde),(Xy,Ce,YZdf),(Xy,Ce,YZdg),(Xy,Ce,YZdh),(Xy,Ce,YZdi),(Xy,Ce,YZdj),(Xy, Ce,YZdk),(Xy,Ce,YZdl),(Xy,Ce,YZdm),(Xy,Ce,YZdn),(Xy,Ce,YZdo),(Xy,Ce,YZdp), (Xy,Ce,YZdq),(Xy,Ce,YZdr),(Xy,Ce,YZds),(Xy,Ce,YZdt),(Xy,Ce,YZdu),(Xy,Ce,YZdv), (Xy,Ce,YZdw),(Xy,Ce,YZdx),(Xy,Ce,YZdy),(Xy,Ce,YZdz),(Xy,Ce,YZea),(Xy,Ce, YZeb),(Xy,Ce,YZec),(Xy,Ce,YZed),(Xy,Ce,YZee),(Xy,Ce,YZef),(Xy,Ce,YZeg),(Xy, Ce,YZeh),(Xy,Ce,YZei),(Xy,Ce,YZej),(Xy,Ce,YZek),(Xy,Ce,YZel),(Xy,Ce,YZem),(Xy, Ce,YZen),(Xy,Ce,YZeo),(Xy,Ce,YZep),(Xy,Ce,YZeq),(Xy,Ce,YZer),(Xy,Ce,YZes), (Xy,Ce,YZet),(Xy,Ce,YZeu),(Xy,Ce,YZev),(Xy,Ce,YZew),(Xy,Ce,YZex),(Xy,Ce,YZey), (Xy,Ce,YZez),(Xy,Ce,YZfa),(Xy,Ce,YZfb),(Xy,Ce,YZfc),(Xy,Ce,YZfd),(Xy,Ce, YZfe),(Xy,Ce,YZff),(Xy,Ce,YZfg),(Xy,Ce,YZfh),(Xy,Ce,YZfi),(Xy,Ce,YZfj),(Xy,Ce, YZfk),(Xy,Ce,YZfl),(Xy,Ce,YZfm),(Xy,Ce,YZfn),(Xy,Ce,YZfo),(Xy,Ce,YZfp),(Xy,Ce, YZfq),(Xy,Ce,YZfr),(Xy,Ce,YZfs),(Xy,Ce,YZft),(Xy,Ce,YZfu),(Xy,Cc,YZfv),(Xy,Ce, YZfw),(Xy,Ce,YZfx),(Xy,Ce,YZfy),(Xy,Ce,YZfz),(Xy,Ce,YZga),(Xy,Ce,YZgb),(Xy, Ce,YZgc),(Xy,Ce,YZgd),(Xy,Ce,YZge),(Xy,Ce,YZgf),(Xy,Ce,YZgg),(Xy,Ce,YZgh), (Xy,Ce,YZgi),(Xy,Ce,YZgj),(Xy,Ce,YZgk),(Xy,Ce,YZgl),(Xy,Ce,YZgm),(Xy,Ce,YZgn), (Xy,Ce,YZgo),(Xy,Ce,YZgp),(Xy,Ce,YZgq),(Xy,Ce,YZgr),(Xy,Ce,YZgs),(Xy,Ce, YZgt),(Xy,Cf,YZa),(Xy,Cf,YZb),(Xy,Cf,YZc),(Xy,Cf,YZd),(Xy,Cf,YZe),(Xy,Cf,YZf), (Xy,Cf,YZg),(Xy,Cf,YZh),(Xy,Cf,YZi),(Xy,Cf,YZj),(Xy,Cf,YZk),(Xy,Cf,YZl),(Xy,Cf, YZm),(Xy,Cf,YZn),(Xy,Cf,YZo),(Xy,Cf,YZp),(Xy,Cf,YZq),(Xy,Cf,YZr),(Xy,Cf,YZs), (Xy,Cf,YZt),(Xy,Cf,YZu),(Xy,Cf,YZv),(Xy,Cf,YZw),(Xy,Cf,YZx),(Xy,Cf,YZy),(Xy, Cf,YZz),(Xy,Cf,YZaa),(Xy,Cf,YZab),(Xy,Cf,YZac),(Xy,Cf,YZad),(Xy,Cf,YZae),(Xy, Cf,YZaf),(Xy,Cf,YZag),(Xy,Cf,YZah),(Xy,Cf,YZai),(Xy,Cf,YZaj),(Xy,Cf,YZak),(Xy, Cf,YZal),(Xy,Cf,YZam),(Xy,Cf,YZan),(Xy,Cf,YZao),(Xy,Cf,YZap),(Xy,Cf,YZaq),(Xy, Cf,YZar),(Xy,Cf,YZas),(Xy,Cf,YZat),(Xy,Cf,YZau),(Xy,Cf,YZav),(Xy,Cf,YZaw), (Xy,Cf,YZax),(Xy,Cf,YZay),(Xy,Cf,YZaz),(Xy,Cf,YZba),(Xy,Cf,YZbb),(Xy,Cf,YZbc), (Xy,Cf,YZbd),(Xy,Cf,YZbe),(Xy,Cf,YZbf),(Xy,Cf,YZbg),(Xy,Cf,YZbh),(Xy,Cf,YZbi), (Xy,Cf,YZbj),(Xy,Cf,YZbk),(Xy,Cf,YZbl),(Xy,Cf,YZbm),(Xy,Cf,YZbn),(Xy,Cf,YZbo), (Xy,Cf,YZbp),(Xy,Cf,YZbq),(Xy,Cf,YZbr),(Xy,Cf,YZbs),(Xy,Cf,YZbt),(Xy,Cf, YZbu),(Xy,Cf,YZbv),(Xy,Cf,YZbw),(Xy,Cf,YZbx),(Xy,Cf,YZby),(Xy,Cf,YZbz),(Xy, Cf,YZca),(Xy,Cf,YZcb),(Xy,Cf,YZcc),(Xy,Cf,YZcd),(Xy,Cf,YZce),(Xy,Cf,YZcf),(Xy, Cf,YZcg),(Xy,Cf,YZch),(Xy,Cf,YZci),(Xy,Cf,YZcj),(Xy,Cf,YZck),(Xy,Cf,YZcl),(Xy, Cf,YZcm),(Xy,Cf,YZcn),(Xy,Cf,YZco),(Xy,Cf,YZcp),(Xy,Cf,YZcq),(Xy,Cf,YZcr),(Xy, Cf,YZcs),(Xy,Cf,YZct),(Xy,Cf,YZcu),(Xy,Cf,YZcv),(Xy,Cf,YZcw),(Xy,Cf,YZcx), (Xy,Cf,YZcy),(Xy,Cf,YZcz),(Xy,Cf,YZda),(Xy,Cf,YZdb),(Xy,Cf,YZdc),(Xy,Cf,YZdd), (Xy,Cf,YZde),(Xy,Cf,YZdf),(Xy,Cf,YZdg),(Xy,Cf,YZdh),(Xy,Cf,YZdi),(Xy,Cf,YZdj), (Xy,Cf,YZdk),(Xy,Cf,YZdl),(Xy,Cf,YZdm),(Xy,Cf,YZdn),(Xy,Cf,YZdo),(Xy,Cf, YZdp),(Xy,Cf,YZdq),(Xy,Cf,YZdr),(Xy,Cf,YZds),(Xy,Cf,YZdt),(Xy,Cf,YZdu),(Xy,Cf, YZdv),(Xy,Cf,YZdw),(Xy,Cf,YZdx),(Xy,Cf,YZdy),(Xy,Cf,YZdz),(Xy,Cf,YZea),(Xy, Cf,YZeb),(Xy,Cf,YZec),(Xy,Cf,YZed),(Xy,Cf,YZee),(Xy,Cf,YZef),(Xy,Cf,YZeg),(Xy, Cf,YZeh),(Xy,Cf,YZei),(Xy,Cf,YZej),(Xy,Cf,YZek),(Xy,Cf,YZel),(Xy,Cf,YZem),(Xy, Cf,YZen),(Xy,Cf,YZeo),(Xy,Cf,YZep),(Xy,Cf,YZeq),(Xy,Cf,YZer),(Xy,Cf,YZes),(Xy, Cf,YZet),(Xy,Cf,YZeu),(Xy,Cf,YZev),(Xy,Cf,YZew),(Xy,Cf,YZex),(Xy,Cf,YZey), (Xy,Cf,YZez),(Xy,Cf,YZfa),(Xy,Cf,YZfb),(Xy,Cf,YZfc),(Xy,Cf,YZfd),(Xy,Cf,YZfe), (Xy,Cf,YZff),(Xy,Cf,YZfg),(Xy,Cf,YZfh),(Xy,cf,YZfi),(Xy,Cf,YZfj),(Xy,Cf,YZfk),(Xy, Cf,YZfl),(Xy,Cf,YZfm),(Xy,Cf,YZfn),(Xy,Cf,YZfo),(Xy,Cf,YZfp),(Xy,Cf,YZfq),(Xy, Cf,YZfr),(Xy,Cf,YZfs),(Xy,Cf,YZft),(Xy,Cf,YZfu),(Xy,Cf,YZfv),(Xy,Cf,YZfw),(Xy, Cf,YZfx),(Xy,Cf,YZfy),(Xy,Cf,YZfz),(Xy,Cf,YZga),(Xy,Cf,YZgb),(Xy,Cf,YZgc), (Xy,Cf,YZgd),(Xy,Cf,YZge),(Xy,Cf,YZgf),(Xy,Cf,YZgg),(Xy,Cf,YZgh),(Xy,Cf,YZgi), (Xy,Cf,YZgj),(Xy,Cf,YZgk),(Xy,Cf,YZgl),(Xy,Cf,YZgm),(Xy,Cf,YZgn),(Xy,Cf,YZgo), (Xy,Cf,YZgp),(Xy,Cf,YZgq),(Xy,Cf,YZgr),(Xy,Cf,YZgs),(Xy,Cf,YZgt), (Xz,Ca,YZa),(Xz,Ca,YZb),(Xz,Ca,YZc),(Xz,Ca,YZd),(Xz,Ca,YZe),(Xz,Ca,YZf),(Xz, Ca,YZg),(Xz,Ca,YZh),(Xz,Ca,YZi),(Xz,Ca,YZj),(Xz,Ca,YZk),(Xz,Ca,YZl),(Xz,Ca,YZm), (Xz,Ca,YZn),(Xz,Ca,YZo),(Xz,Ca,YZp),(Xz,Ca,YZq),(Xz,Ca,YZr),(Xz,Ca,YZs), (Xz,Ca,YZt),(Xz,Ca,YZu),(Xz,Ca,YZv),(Xz,Ca,YZw),(Xz,Ca,YZx),(Xz,Ca,YZy),(Xz, Ca,YZz),(Xz,Ca,YZaa),(Xz,Ca,YZab),(Xz,Ca,YZac),(Xz,Ca,YZad),(Xz,Ca,YZae),(Xz, Ca,YZaf),(Xz,Ca,YZag),(Xz,Ca,YZah),(Xz,Ca,YZai),(Xz,Ca,YZaj),(Xz,Ca,YZak),(Xz, Ca,YZal),(Xz,Ca,YZam),(Xz,Ca,YZan),(Xz,Ca,YZao),(Xz,Ca,YZap),(Xz,Ca,YZaq), (Xz,Ca,YZar),(Xz,Ca,YZas),(Xz,Ca,YZat),(Xz,Ca,YZau),(Xz,Ca,YZav),(Xz,Ca,YZaw), (Xz,Ca,YZax),(Xz,Ca,YZay),(Xz,Ca,YZaz),(Xz,Ca,YZba),(Xz,Ca,YZbb),(Xz,Ca,YZbc), (Xz,Ca,YZbd),(Xz,Ca,YZbe),(Xz,Ca,YZbf),(Xz,Ca,YZbg),(Xz,Ca,YZbh),(Xz,Ca, YZbi),(Xz,Ca,YZbj),(Xz,Ca,YZbk),(Xz,Ca,YZbl),(Xz,Ca,YZbm),(Xz,Ca,YZbn),(Xz, Ca,YZbo),(Xz,Ca,YZbp),(Xz,Ca,YZbq),(Xz,Ca,YZbr),(Xz,Ca,YZbs),(Xz,Ca,YZbt),(Xz, Ca,YZbu),(Xz,Ca,YZbv),(Xz,Ca,YZbw),(Xz,Ca,YZbx),(Xz,Ca,YZby),(Xz,Ca,YZbz), (Xz,Ca,YZca),(Xz,Ca,YZcb),(Xz,Ca,YZcc),(Xz,Ca,YZcd),(Xz,Ca,YZce),(Xz,Ca,YZcf), (Xz,Ca,YZcg),(Xz,Ca,YZch),(Xz,Ca,YZci),(Xz,Ca,YZcj),(Xz,Ca,YZck),(Xz,Ca,YZcl), (Xz,Ca,YZcm),(Xz,Ca,YZcn),(Xz,Ca,YZco),(Xz,Ca,YZcp),(Xz,Ca,YZcq),(Xz,Ca, YZcr),(Xz,Ca,YZcs),(Xz,Ca,YZct),(Xz,Ca,YZcu),(Xz,Ca,YZcv),(Xz,Ca,YZcw),(Xz, Ca,YZcx),(Xz,Ca,YZcy),(Xz,Ca,YZcz),(Xz,Ca,YZda),(Xz,Ca,YZdb),(Xz,Ca,YZdc),(Xz, Ca,YZdd),(Xz,Ca,YZde),(Xz,Ca,YZdf),(Xz,Ca,YZdg),(Xz,Ca,YZdh),(Xz,Ca,YZdi), (Xz,Ca,YZdj),(Xz,Ca,YZdk),(Xz,Ca,YZdl),(Xz,Ca,YZdm),(Xz,Ca,YZdn),(Xz,Ca,YZdo), (Xz,Ca,YZdp),(Xz,Ca,YZdq),(Xz,Ca,YZdr),(Xz,Ca,YZds),(Xz,Ca,YZdt),(Xz,Ca,YZdu), (Xz,Ca,YZdv),(Xz,Ca,YZdw),(Xz,Ca,YZdx),(Xz,Ca,YZdy),(Xz,Ca,YZdz),(Xz,Ca, YZea),(Xz,Ca,YZeb),(Xz,Ca,YZec),(Xz,Ca,YZed),(Xz,Ca,YZee),(Xz,Ca,YZef),(Xz, Ca,YZeg),(Xz,Ca,YZeh),(Xz,Ca,YZei),(Xz,Ca,YZej),(Xz,Ca,YZek),(Xz,Ca,YZel),(Xz, Ca,YZem),(Xz,Ca,YZen),(Xz,Ca,YZeo),(Xz,Ca,YZep),(Xz,Ca,YZeq),(Xz,Ca,YZer),(Xz, Ca,YZes),(Xz,Ca,YZet),(Xz,Ca,YZeu),(Xz,Ca,YZev),(Xz,Ca,YZew),(Xz,Ca,YZex), (Xz,Ca,YZey),(Xz,Ca,YZez),(Xz,Ca,YZfa),(Xz,Ca,YZfb),(Xz,Ca,YZfc),(Xz,Ca,YZfd), (Xz,Ca,YZfe),(Xz,Ca,YZff),(Xz,Ca,YZfg),(Xz,Ca,YZfh),(Xz,Ca,YZfi),(Xz,Ca,YZfj), (Xz,Ca,YZfk),(Xz,Ca,YZfl),(Xz,Ca,YZfm),(Xz,Ca,YZfn),(Xz,Ca,YZfo),(Xz,Ca,YZfp), (Xz,Ca,YZfq),(Xz,Ca,YZfr),(Xz,Ca,YZfs),(Xz,Ca,YZft),(Xz,Ca,YZfu),(Xz,Ca,YZfv), (Xz,Ca,YZfw),(Xz,Ca,YZfx),(Xz,Ca,YZfy),(Xz,Ca,YZfz),(Xz,Ca,YZga),(Xz,Ca,YZgb), (Xz,Ca,YZgc),(Xz,Ca,YZgd),(Xz,Ca,YZge),(Xz,Ca,YZgf),(Xz,Ca,YZgg),(Xz,Ca, YZgh),(Xz,Ca,YZgi),(Xz,Ca,YZgj),(Xz,Ca,YZgk),(Xz,Ca,YZgl),(Xz,Ca,YZgm),(Xz, Ca,YZgn),(Xz,Ca,YZgo),(Xz,Ca,YZgp),(Xz,Ca,YZgq),(Xz,Ca,YZgr),(Xz,Ca,YZgs),(Xz, Ca,YZgt),(Xz,Cb,YZa),(Xz,Cb,YZb),(Xz,Cb,YZc),(Xz,Cb,YZd),(Xz,Cb,YZe),(Xz, Cb,YZf),(Xz,Cb,YZg),(Xz,Cb,YZh),(Xz,Cb,YZi),(Xz,Cb,YZj),(Xz,Cb,YZk),(Xz,Cb, YZl),(Xz,Cb,YZm),(Xz,Cb,YZn),(Xz,Cb,YZo),(Xz,Cb,YZp),(Xz,Cb,YZq),(Xz,Cb,YZr), (Xz,Cb,YZs),(Xz,Cb,YZt),(Xz,Cb,YZu),(Xz,Cb,YZv),(Xz,Cb,YZw),(Xz,Cb,YZx),(Xz, Cb,YZy),(Xz,Cb,YZz),(Xz,Cb,YZaa),(Xz,Cb,YZab),(Xz,Cb,YZac),(Xz,Cb,YZad), (Xz,Cb,YZae),(Xz,Cb,YZaf),(Xz,Cb,YZag),(Xz,Cb,YZah),(Xz,Cb,YZai),(Xz,Cb,YZaj), (Xz,Cb,YZak),(Xz,Cb,YZal),(Xz,Cb,YZam),(Xz,Cb,YZan),(Xz,Cb,YZao),(Xz,Cb,YZap), (Xz,Cb,YZaq),(Xz,Cb,YZar),(Xz,Cb,YZas),(Xz,Cb,YZat),(Xz,Cb,YZau),(Xz,Cb, YZav),(Xz,Cb,YZaw),(Xz,Cb,YZax),(Xz,Cb,YZay),(Xz,Cb,YZaz),(Xz,Cb,YZba),(Xz, Cb,YZbb),(Xz,Cb,YZbc),(Xz,Cb,YZbd),(Xz,Cb,YZbe),(Xz,Cb,YZbf),(Xz,Cb,YZbg),(Xz, Cb,YZbh),(Xz,Cb,YZbi),(Xz,Cb,YZbj),(Xz,Cb,YZbk),(Xz,Cb,YZbl),(Xz,Cb,YZbm), (Xz,Cb,YZbn),(Xz,Cb,YZbo),(Xz,Cb,YZbp),(Xz,Cb,YZbq),(Xz,Cb,YZbr),(Xz,Cb, YZbs),(Xz,Cb,YZbt),(Xz,Cb,YZbu),(Xz,Cb,YZbv),(Xz,Cb,YZbw),(Xz,Cb,YZbx),(Xz, Cb,YZby),(Xz,Cb,YZbz),(Xz,Cb,YZca),(Xz,Cb,YZcb),(Xz,Cb,YZcc),(Xz,Cb,YZcd),(Xz, Cb,YZce),(Xz,Cb,YZcf),(Xz,Cb,YZcg),(Xz,Cb,YZch),(Xz,Cb,YZci),(Xz,Cb,YZcj), (Xz,Cb,YZck),(Xz,Cb,YZcl),(Xz,Cb,YZcm),(Xz,Cb,YZcn),(Xz,Cb,YZco),(Xz,Cb,YZcp), (Xz,Cb,YZcq),(Xz,Cb,YZcr),(Xz,Cb,YZcs),(Xz,Cb,YZct),(Xz,Cb,YZcu),(Xz,Cb, YZcv),(Xz,Cb,YZcw),(Xz,Cb,YZcx),(Xz,Cb,YZcy),(Xz,Cb,YZcz),(Xz,Cb,YZda),(Xz, Cb,YZdb),(Xz,Cb,YZdc),(Xz,Cb,YZdd),(Xz,Cb,YZde),(Xz,Cb,YZdf),(Xz,Cb,YZdg),(Xz, Cb,YZdh),(Xz,Cb,YZdi),(Xz,Cb,YZdj),(Xz,Cb,YZdk),(Xz,Cb,YZdl),(Xz,Cb,YZdm), (Xz,Cb,YZdn),(Xz,Cb,YZdo),(Xz,Cb,YZdp),(Xz,Cb,YZdq),(Xz,Cb,YZdr),(Xz,Cb, YZds),(Xz,Cb,YZdt),(Xz,Cb,YZdu),(Xz,Cb,YZdv),(Xz,Cb,YZdw),(Xz,Cb,YZdx),(Xz, Cb,YZdy),(Xz,Cb,YZdz),(Xz,Cb,YZea),(Xz,Cb,YZeb),(Xz,Cb,YZec),(Xz,Cb,YZed),(Xz, Cb,YZee),(Xz,Cb,YZef),(Xz,Cb,YZeg),(Xz,Cb,YZeh),(Xz,Cb,YZei),(Xz,Cb,YZej), (Xz,Cb,YZek),(Xz,Cb,YZel),(Xz,Cb,YZem),(Xz,Cb,YZen),(Xz,Cb,YZeo),(Xz,Cb,YZep), (Xz,Cb,YZeq),(Xz,Cb,YZer),(Xz,Cb,YZes),(Xz,Cb,YZet),(Xz,Cb,YZeu),(Xz,Cb, YZev),(Xz,Cb,YZew),(Xz,Cb,YZex),(Xz,Cb,YZey),(Xz,Cb,YZez),(Xz,Cb,YZfa),(Xz, Cb,YZfb),(Xz,Cb,YZfc),(Xz,Cb,YZfd),(Xz,Cb,YZfe),(Xz,Cb,YZff),(Xz,Cb,YZfg),(Xz, Cb,YZfh),(Xz,Cb,YZfi),(Xz,Cb,YZfj),(Xz,Cb,YZfk),(Xz,Cb,YZfl),(Xz,Cb,YZfm),(Xz, Cb,YZfn),(Xz,Cb,YZfo),(Xz,Cb,YZfp),(Xz,Cb,YZfq),(Xz,Cb,YZfr),(Xz,Cb,YZfs),(Xz, Cb,YZft),(Xz,Cb,YZfu),(Xz,Cb,YZfv),(Xz,Cb,YZfw),(Xz,Cb,YZfx),(Xz,Cb,YZfy), (Xz,Cb,YZfz),(Xz,Cb,YZga),(Xz,Cb,YZgb),(Xz,Cb,YZgc),(Xz,Cb,YZgd),(Xz,Cb,YZge), (Xz,Cb,YZgf),(Xz,Cb,YZgg),(Xz,Cb,YZgh),(Xz,Cb,YZgi),(Xz,Cb,YZgj),(Xz,Cb, YZgk),(Xz,Cb,YZgl),(Xz,Cb,YZgm),(Xz,Cb,YZgn),(Xz,Cb,YZgo),(Xz,Cb,YZgp),(Xz, Cb,YZgq),(Xz,Cb,YZgr),(Xz,Cb,YZgs),(Xz,Cb,YZgt),(Xz,Cc,YZa),(Xz,Cc,YZb),(Xz, Cc,YZc),(Xz,Cc,YZd),(Xz,Cc,YZe),(Xz,Cc,YZf),(Xz,Cc,YZg),(Xz,Cc,YZh),(Xz,Cc, YZi),(Xz,Cc,YZj),(Xz,Cc,YZk),(Xz,Cc,YZl),(Xz,Cc,YZm),(Xz,Cc,YZn),(Xz,Cc,YZo), (Xz,Cc,YZp),(Xz,Cc,YZq),(Xz,Cc,YZr),(Xz,Cc,YZs),(Xz,Cc,YZt),(Xz,Cc,YZu),(Xz, Cc,YZv),(Xz,Cc,YZw),(Xz,Cc,YZx),(Xz,Cc,YZy),(Xz,Cc,YZz),(Xz,Cc,YZaa),(Xz,Cc, YZab),(Xz,Cc,YZac),(Xz,Cc,YZad),(Xz,Cc,YZae),(Xz,Cc,YZaf),(Xz,Cc,YZag),(Xz, Cc,YZah),(Xz,Cc,YZai),(Xz,Cc,YZaj),(Xz,Cc,YZak),(Xz,Cc,YZal),(Xz,Cc,YZam),(Xz, Cc,YZan),(Xz,Cc,YZao),(Xz,Cc,YZap),(Xz,Cc,YZaq),(Xz,Cc,YZar),(Xz,Cc,YZas),(Xz, Cc,YZat),(Xz,Cc,YZau),(Xz,Cc,YZav),(Xz,Cc,YZaw),(Xz,Cc,YZax),(Xz,Cc,YZay), (Xz,Cc,YZaz),(Xz,Cc,YZba),(Xz,Cc,YZbb),(Xz,Cc,YZbc),(Xz,Cc,YZbd),(Xz,Cc,YZbe), (Xz,Cc,YZbf),(Xz,Cc,YZbg),(Xz,Cc,YZbh),(Xz,Cc,YZbi),(Xz,Cc,YZbj),(Xz,Cc,YZbk), (Xz,Cc,YZbl),(Xz,Cc,YZbm),(Xz,Cc,YZbn),(Xz,Cc,YZbo),(Xz,Cc,YZbp),(Xz,Cc, YZbq),(Xz,Cc,YZbr),(Xz,Cc,YZbs),(Xz,Cc,YZbt),(Xz,Cc,YZbu),(Xz,Cc,YZbv),(Xz, Cc,YZbw),(Xz,Cc,YZbx),(Xz,Cc,YZby),(Xz,Cc,YZbz),(Xz,Cc,YZca),(Xz,Cc,YZcb),(Xz, Cc,YZcc),(Xz,Cc,YZcd),(Xz,Cc,YZce),(Xz,Cc,YZcf),(Xz,Cc,YZcg),(Xz,Cc,YZch), (Xz,Cc,YZci),(Xz,Cc,YZcj),(Xz,Cc,YZck),(Xz,Cc,YZcl),(Xz,Cc,YZcm),(Xz,Cc,YZcn), (Xz,Cc,YZco),(Xz,Cc,YZcp),(Xz,Cc,YZcq),(Xz,Cc,YZcr),(Xz,Cc,YZcs),(Xz,Cc,YZct), (Xz,Cc,YZcu),(Xz,Cc,YZcv),(Xz,Cc,YZcw),(Xz,Cc,YZcx),(Xz,Cc,YZcy),(Xz,Cc, YZcz),(Xz,Cc,YZda),(Xz,Cc,YZdb),(Xz,Cc,YZdc),(Xz,Cc,YZdd),(Xz,Cc,YZde),(Xz, Cc,YZdf),(Xz,Cc,YZdg),(Xz,Cc,YZdh),(Xz,Cc,YZdi),(Xz,Cc,YZdj),(Xz,Cc,YZdk),(Xz, Cc,YZdl),(Xz,Cc,YZdm),(Xz,Cc,YZdn),(Xz,Cc,YZdo),(Xz,Cc,YZdp),(Xz,Cc,YZdq), (Xz,Cc,YZdr),(Xz,Cc,YZds),(Xz,Cc,YZdt),(Xz,Cc,YZdu),(Xz,Cc,YZdv),(Xz,Cc,YZdw), (Xz,Cc,YZdx),(Xz,Cc,YZdy),(Xz,Cc,YZdz),(Xz,Cc,YZea),(Xz,Cc,YZeb),(Xz,Cc, YZec),(Xz,Cc,YZed),(Xz,Cc,YZee),(Xz,Cc,YZef),(Xz,Cc,YZeg),(Xz,Cc,YZeh),(Xz,Cc, YZei),(Xz,Cc,YZej),(Xz,Cc,YZek),(Xz,Cc,YZel),(Xz,Cc,YZem),(Xz,Cc,YZen),(Xz, Cc,YZeo),(Xz,Cc,YZep),(Xz,Cc,YZeq),(Xz,Cc,YZer),(Xz,Cc,YZes),(Xz,Cc,YZet),(Xz, Cc,YZeu),(Xz,Cc,YZev),(Xz,Cc,YZew),(Xz,Cc,YZex),(Xz,Cc,YZey),(Xz,Cc,YZez), (Xz,Cc,YZfa),(Xz,Cc,YZfb),(Xz,Cc,YZfc),(Xz,Cc,YZfd),(Xz,Cc,YZfe),(Xz,Cc,YZff), (Xz,Cc,YZfg),(Xz,Cc,YZfh),(Xz,Cc,YZfi),(Xz,Cc,YZfj),(Xz,Cc,YZfk),(Xz,Cc,YZfl),(Xz, Cc,YZfm),(Xz,Cc,YZfn),(Xz,Cc,YZfo),(Xz,Cc,YZfp),(Xz,Cc,YZfq),(Xz,Cc,YZfr), (Xz,Cc,YZfs),(Xz,Cc,YZft),(Xz,Cc,YZfu),(Xz,Cc,YZfv),(Xz,Cc,YZfw),(Xz,Cc,YZfx), (Xz,Cc,YZfy),(Xz,Cc,YZfz),(Xz,Cc,YZga),(Xz,Cc,YZgb),(Xz,Cc,YZgc),(Xz,Cc,YZgd), (Xz,Cc,YZge),(Xz,Cc,YZgf),(Xz,Cc,YZgg),(Xz,Cc,YZgh),(Xz,Cc,YZgi),(Xz,Cc,YZgj), (Xz,Cc,YZgk),(Xz,Cc,YZgl),(Xz,Cc,YZgm),(Xz,Cc,YZgn),(Xz,Cc,YZgo),(Xz,Cc, YZgp),(Xz,Cc,YZgq),(Xz,Cc,YZgr),(Xz,Cc,YZgs),(Xz,Cc,YZgt),(Xz,Cd,YZa),(Xz, Cd,YZb),(Xz,Cd,YZc),(Xz,Cd,YZd),(Xz,Cd,YZe),(Xz,Cd,YZf),(Xz,Cd,YZg),(Xz,Cd, YZh),(Xz,Cd,YZi),(Xz,Cd,YZj),(Xz,Cd,YZk),(Xz,Cd,YZl),(Xz,Cd,YZm),(Xz,Cd,YZn), (Xz,Cd,YZo),(Xz,Cd,YZp),(Xz,Cd,YZq),(Xz,Cd,YZr),(Xz,Cd,YZs),(Xz,Cd,YZt),(Xz, Cd,YZu),(Xz,Cd,YZv),(Xz,Cd,YZw),(Xz,Cd,YZx),(Xz,Cd,YZy),(Xz,Cd,YZz),(Xz, Cd,YZaa),(Xz,Cd,YZab),(Xz,Cd,YZac),(Xz,Cd,YZad),(Xz,Cd,YZae),(Xz,Cd,YZaf),(Xz, Cd,YZag),(Xz,Cd,YZah),(Xz,Cd,YZai),(Xz,Cd,YZaj),(Xz,Cd,YZak),(Xz,Cd,YZal), (Xz,Cd,YZam),(Xz,Cd,YZan),(Xz,Cd,YZao),(Xz,Cd,YZap),(Xz,Cd,YZaq),(Xz,Cd,YZar), (Xz,Cd,YZas),(Xz,Cd,YZat),(Xz,Cd,YZau),(Xz,Cd,YZav),(Xz,Cd,YZaw),(Xz,Cd, YZax),(Xz,Cd,YZay),(Xz,Cd,YZaz),(Xz,Cd,YZba),(Xz,Cd,YZbb),(Xz,Cd,YZbc),(Xz, Cd,YZbd),(Xz,Cd,YZbe),(Xz,Cd,YZbf),(Xz,Cd,YZbg),(Xz,Cd,YZbh),(Xz,Cd,YZbi),(Xz, Cd,YZbj),(Xz,Cd,YZbk),(Xz,Cd,YZbl),(Xz,Cd,YZbm),(Xz,Cd,YZbn),(Xz,Cd,YZbo), (Xz,Cd,YZbp),(Xz,Cd,YZbq),(Xz,Cd,YZbr),(Xz,Cd,YZbs),(Xz,Cd,YZbt),(Xz,Cd,YZbu), (Xz,Cd,YZbv),(Xz,Cd,YZbw),(Xz,Cd,YZbx),(Xz,Cd,YZby),(Xz,Cd,YZbz),(Xz, Cd,YZca),(Xz,Cd,YZcb),(Xz,Cd,YZcc),(Xz,Cd,YZcd),(Xz,Cd,YZce),(Xz,Cd,YZcf),(Xz, Cd,YZcg),(Xz,Cd,YZch),(Xz,Cd,YZci),(Xz,Cd,YZcj),(Xz,Cd,YZck),(Xz,Cd,YZcl), (Xz,Cd,YZcm),(Xz,Cd,YZcn),(Xz,Cd,YZco),(Xz,Cd,YZcp),(Xz,Cd,YZcq),(Xz,Cd,YZcr), (Xz,Cd,YZcs),(Xz,Cd,YZct),(Xz,Cd,YZcu),(Xz,Cd,YZcv),(Xz,Cd,YZcw),(Xz,Cd, YZcx),(Xz,Cd,YZcy),(Xz,Cd,YZcz),(Xz,Cd,YZda),(Xz,Cd,YZdb),(Xz,Cd,YZdc),(Xz, Cd,YZdd),(Xz,Cd,YZde),(Xz,Cd,YZdf),(Xz,Cd,YZdg),(Xz,Cd,YZdh),(Xz,Cd,YZdi),(Xz, Cd,YZdj),(Xz,Cd,YZdk),(Xz,Cd,YZdl),(Xz,Cd,YZdm),(Xz,Cd,YZdn),(Xz,Cd,YZdo), (Xz,Cd,YZdp),(Xz,Cd,YZdq),(Xz,Cd,YZdr),(Xz,Cd,YZds),(Xz,Cd,YZdt),(Xz,Cd,YZdu), (Xz,Cd,YZdv),(Xz,Cd,YZdw),(Xz,Cd,YZdx),(Xz,Cd,YZdy),(Xz,Cd,YZdz),(Xz, Cd,YZea),(Xz,Cd,YZeb),(Xz,Cd,YZec),(Xz,Cd,YZed),(Xz,Cd,YZee),(Xz,Cd,YZef),(Xz, Cd,YZeg),(Xz,Cd,YZeh),(Xz,Cd,YZei),(Xz,Cd,YZej),(Xz,Cd,YZek),(Xz,Cd,YZel), (Xz,Cd,YZem),(Xz,Cd,YZen),(Xz,Cd,YZeo),(Xz,Cd,YZep),(Xz,Cd,YZeq),(Xz,Cd,YZer), (Xz,Cd,YZes),(Xz,Cd,YZet),(Xz,Cd,YZeu),(Xz,Cd,YZev),(Xz,Cd,YZew),(Xz,Cd, YZex),(Xz,Cd,YZey),(Xz,Cd,YZez),(Xz,Cd,YZfa),(Xz,Cd,YZfb),(Xz,Cd,YZfc),(Xz, Cd,YZfd),(Xz,Cd,YZfe),(Xz,Cd,YZff),(Xz,Cd,YZfg),(Xz,Cd,YZfh),(Xz,Cd,YZfi),(Xz, Cd,YZfj),(Xz,Cd,YZfk),(Xz,Cd,YZfl),(Xz,Cd,YZfm),(Xz,Cd,YZfn),(Xz,Cd,YZfo),(Xz, Cd,YZfp),(Xz,Cd,YZfq),(Xz,Cd,YZfr),(Xz,Cd,YZfs),(Xz,Cd,YZft),(Xz,Cd,YZfu),(Xz, Cd,YZfv),(Xz,Cd,YZfw),(Xz,Cd,YZfx),(Xz,Cd,YZfy),(Xz,Cd,YZfz),(Xz,Cd,YZga), (Xz,Cd,YZgb),(Xz,Cd,YZgc),(Xz,Cd,YZgd),(Xz,Cd,YZge),(Xz,Cd,YZgf),(Xz,Cd,YZgg), (Xz,Cd,YZgh),(Xz,Cd,YZgi),(Xz,Cd,YZgj),(Xz,Cd,YZgk),(Xz,Cd,YZgl),(Xz,Cd, YZgm),(Xz,Cd,YZgn),(Xz,Cd,YZgo),(Xz,Cd,YZgp),(Xz,Cd,YZgq),(Xz,Cd,YZgr),(Xz, Cd,YZgs),(Xz,Cd,YZgt),(Xz,Ce,YZa),(Xz,Ce,YZb),(Xz,Ce,YZc),(Xz,Ce,YZd),(Xz,Ce, YZe),(Xz,Ce,YZf),(Xz,Ce,YZg),(Xz,Ce,YZh),(Xz,Ce,YZi),(Xz,Ce,YZj),(Xz,Ce,YZk), (Xz,Ce,YZl),(Xz,Ce,YZm),(Xz,Ce,YZn),(Xz,Ce,YZo),(Xz,Ce,YZp),(Xz,Ce,YZq),(Xz, Ce,YZr),(Xz,Ce,YZs),(Xz,Ce,YZt),(Xz,Ce,YZu),(Xz,Ce,YZv),(Xz,Ce,YZw),(Xz,Ce, YZx),(Xz,Ce,YZy),(Xz,Ce,YZz),(Xz,Ce,YZaa),(Xz,Ce,YZab),(Xz,Ce,YZac),(Xz,Ce, YZad),(Xz,Ce,YZae),(Xz,Ce,YZaf),(Xz,Ce,YZag),(Xz,Ce,YZah),(Xz,Ce,YZai),(Xz,Ce, YZaj),(Xz,Ce,YZak),(Xz,Ce,YZal),(Xz,Ce,YZam),(Xz,Ce,YZan),(Xz,Ce,YZao),(Xz, Ce,YZap),(Xz,Ce,YZaq),(Xz,Ce,YZar),(Xz,Ce,YZas),(Xz,Ce,YZat),(Xz,Ce,YZau),(Xz, Ce,YZav),(Xz,Ce,YZaw),(Xz,Ce,YZax),(Xz,Ce,YZay),(Xz,Ce,YZaz),(Xz,Ce,YZba), (Xz,Ce,YZbb),(Xz,Ce,YZbc),(Xz,Ce,YZbd),(Xz,Ce,YZbe),(Xz,Ce,YZbf),(Xz,Ce,YZbg), (Xz,Ce,YZbh),(Xz,Ce,YZbi),(Xz,Ce,YZbj),(Xz,Ce,YZbk),(Xz,Ce,YZbl),(Xz,Ce,YZbm), (Xz,Ce,YZbn),(Xz,Ce,YZbo),(Xz,Ce,YZbp),(Xz,Ce,YZbq),(Xz,Ce,YZbr),(Xz,Ce, YZbs),(Xz,Ce,YZbt),(Xz,Ce,YZbu),(Xz,Ce,YZbv),(Xz,Ce,YZbw),(Xz,Ce,YZbx),(Xz, Ce,YZby),(Xz,Ce,YZbz),(Xz,Ce,YZca),(Xz,Ce,YZcb),(Xz,Ce,YZcc),(Xz,Ce,YZcd),(Xz, Ce,YZce),(Xz,Ce,YZcf),(Xz,Ce,YZcg),(Xz,Ce,YZch),(Xz,Ce,YZci),(Xz,Ce,YZcj), (Xz,Ce,YZck),(Xz,Ce,YZcl),(Xz,Ce,YZcm),(Xz,Ce,YZcn),(Xz,Ce,YZco),(Xz,Ce,YZcp), (Xz,Ce,YZcq),(Xz,Ce,YZcr),(Xz,Ce,YZcs),(Xz,Ce,YZct),(Xz,CC,YZcu),(Xz,Ce,YZcv), (Xz,Ce,YZcw),(Xz,Ce,YZcx),(Xz,Ce,YZcy),(Xz,Ce,YZcz),(Xz,Ce,YZda),(Xz,Ce, YZdb),(Xz,Ce,YZdc),(Xz,Ce,YZdd),(Xz,Ce,YZde),(Xz,Ce,YZdf),(Xz,Ce,YZdg),(Xz, Ce,YZdh),(Xz,Ce,YZdi),(Xz,Ce,YZdj),(Xz,Ce,YZdk),(Xz,Ce,YZdl),(Xz,Ce,YZdm),(Xz, Ce,YZdn),(Xz,Ce,YZdo),(Xz,Ce,YZdp),(Xz,Ce,YZdq),(Xz,Ce,YZdr),(Xz,Ce,YZds), (Xz,Ce,YZdt),(Xz,Ce,YZdu),(Xz,Ce,YZdv),(Xz,Ce,YZdw),(Xz,Ce,YZdx),(Xz,Ce,YZdy), (Xz,Ce,YZdz),(Xz,Ce,YZea),(Xz,Ce,YZeb),(Xz,Ce,YZec),(Xz,Ce,YZed),(Xz,Ce, YZee),(Xz,Ce,YZef),(Xz,Ce,YZeg),(Xz,Ce,YZeh),(Xz,Ce,YZei),(Xz,Ce,YZej),(Xz,Ce, YZek),(Xz,Ce,YZel),(Xz,Ce,YZem),(Xz,Ce,YZen),(Xz,Ce,YZeo),(Xz,Ce,YZep),(Xz, Ce,YZeq),(Xz,Ce,YZer),(Xz,Ce,YZes),(Xz,Ce,YZet),(Xz,Ce,YZeu),(Xz,Ce,YZev),(Xz, Ce,YZew),(Xz,Ce,YZex),(Xz,Ce,YZey),(Xz,Ce,YZez),(Xz,Ce,YZfa),(Xz,Ce,YZfb),(Xz, Ce,YZfc),(Xz,Ce,YZfd),(Xz,Ce,YZfe),(Xz,Ce,YZff),(Xz,Ce,YZfg),(Xz,Ce,YZfh),(Xz, Ce,YZfi),(Xz,Ce,YZfj),(Xz,Ce,YZfk),(Xz,Ce,YZfl),(Xz,Ce,YZfm),(Xz,Ce,YZfn),(Xz, Ce,YZfo),(Xz,Ce,YZfp),(Xz,Ce,YZfq),(Xz,Ce,YZfr),(Xz,Ce,YZfs),(Xz,Ce,YZft),(Xz, Ce,YZfu),(Xz,Ce,YZfv),(Xz,Ce,YZfw),(Xz,Ce,YZfx),(Xz,Ce,YZfy),(Xz,Ce,YZfz), (Xz,Ce,YZga),(Xz,Ce,YZgb),(Xz,Ce,YZgc),(Xz,Ce,YZgd),(Xz,Ce,YZge),(Xz,Ce,YZgf), (Xz,Ce,YZgg),(Xz,Ce,YZgh),(Xz,Ce,YZgi),(Xz,Ce,YZgj),(Xz,Ce,YZgk),(Xz,Ce,YZgl), (Xz,Ce,YZgm),(Xz,Ce,YZgn),(Xz,Ce,YZgo),(Xz,Ce,YZgp),(Xz,Ce,YZgq),(Xz,Ce, YZgr),(Xz,Ce,YZgs),(Xz,Ce,YZgt),(Xz,Cf,YZa),(Xz,Cf,YZb),(Xz,Cf,YZc),(Xz,Cf, YZd),(Xz,Cf,YZe),(Xz,Cf,YZf),(Xz,Cf,YZg),(Xz,Cf,YZh),(Xz,Cf,YZi),(Xz,Cf,YZj),(Xz, Cf,YZk),(Xz,Cf,YZl),(Xz,Cf,YZm),(Xz,Cf,YZn),(Xz,Cf,YZo),(Xz,Cf,YZp),(Xz,Cf, YZq),(Xz,Cf,YZr),(Xz,Cf,YZs),(Xz,Cf,YZt),(Xz,Cf,YZu),(Xz,Cf,YZv),(Xz,Cf,YZw), (Xz,Cf,YZx),(Xz,Cf,YZy),(Xz,Cf,YZz),(Xz,Cf,YZaa),(Xz,Cf,YZab),(Xz,Cf,YZac),(Xz, Cf,YZad),(Xz,Cf,YZae),(Xz,Cf,YZaf),(Xz,Cf,YZag),(Xz,Cf,YZah),(Xz,Cf,YZai),(Xz, Cf,YZaj),(Xz,Cf,YZak),(Xz,Cf,YZal),(Xz,Cf,YZam),(Xz,Cf,YZan),(Xz,Cf,YZao),(Xz, Cf,YZap),(Xz,Cf,YZaq),(Xz,Cf,YZar),(Xz,Cf,YZas),(Xz,Cf,YZat),(Xz,Cf,YZau),(Xz, Cf,YZav),(Xz,Cf,YZaw),(Xz,Cf,YZax),(Xz,Cf,YZay),(Xz,Cf,YZaz),(Xz,Cf,YZba),(Xz, Cf,YZbb),(Xz,Cf,YZbc),(Xz,Cf,YZbd),(Xz,Cf,YZbe),(Xz,Cf,YZbf),(Xz,Cf,YZbg), (Xz,Cf,YZbh),(Xz,Cf,YZbi),(Xz,Cf,YZbj),(Xz,Cf,YZbk),(Xz,Cf,YZbl),(Xz,Cf,YZbm), (Xz,Cf,YZbn),(Xz,Cf,YZbo),(Xz,Cf,YZbp),(Xz,Cf,YZbq),(Xz,Cf,YZbr),(Xz,Cf,YZbs), (Xz,Cf,YZbt),(Xz,Cf,YZbu),(Xz,Cf,YZbv),(Xz,Cf,YZbw),(Xz,Cf,YZbx),(Xz,Cf,YZby), (Xz,Cf,YZbz),(Xz,Cf,YZca),(Xz,Cf,YZcb),(Xz,Cf,YZcc),(Xz,Cf,YZcd),(Xz,Cf,YZce), (Xz,Cf,YZcf),(Xz,Cf,YZcg),(Xz,Cf,YZch),(Xz,Cf,YZci),(Xz,Cf,YZcj),(Xz,Cf,YZck), (Xz,Cf,YZcl),(Xz,Cf,YZcm),(Xz,Cf,YZcn),(Xz,Cf,YZco),(Xz,Cf,YZcp),(Xz,Cf,YZcq), (Xz,Cf,YZcr),(Xz,Cf,YZcs),(Xz,Cf,YZct),(Xz,Cf,YZcu),(Xz,Cf,YZcv),(Xz,Cf,YZcw), (Xz,Cf,YZcx),(Xz,Cf,YZcy),(Xz,Cf,YZcz),(Xz,Cf,YZda),(Xz,Cf,YZdb),(Xz,Cf,YZdc), (Xz,Cf,YZdd),(Xz,Cf,YZde),(Xz,Cf,YZdf),(Xz,Cf,YZdg),(Xz,Cf,YZdh),(Xz,Cf, YZdi),(Xz,Cf,YZdj),(Xz,Cf,YZdk),(Xz,Cf,YZdl),(Xz,Cf,YZdm),(Xz,Cf,YZdn),(Xz,Cf, YZdo),(Xz,Cf,YZdp),(Xz,Cf,YZdq),(Xz,Cf,YZdr),(Xz,Cf,YZds),(Xz,Cf,YZdt),(Xz,Cf, YZdu),(Xz,Cf,YZdv),(Xz,Cf,YZdw),(Xz,Cf,YZdx),(Xz,Cf,YZdy),(Xz,Cf,YZdz),(Xz, Cf,YZea),(Xz,Cf,YZeb),(Xz,Cf,YZec),(Xz,Cf,YZed),(Xz,Cf,YZee),(Xz,Cf,YZef),(Xz, Cf,YZeg),(Xz,Cf,YZeh),(Xz,Cf,YZei),(Xz,Cf,YZej),(Xz,Cf,YZek),(Xz,Cf,YZel),(Xz, Cf,YZem),(Xz,Cf,YZen),(Xz,Cf,YZeo),(Xz,Cf,YZep),(Xz,Cf,YZeq),(Xz,Cf,YZer),(Xz, Cf,YZes),(Xz,Cf,YZet),(Xz,Cf,YZeu),(Xz,Cf,YZev),(Xz,Cf,YZew),(Xz,Cf,YZex),(Xz, Cf,YZey),(Xz,Cf,YZez),(Xz,Cf,YZfa),(Xz,Cf,YZfb),(Xz,Cf,YZfc),(Xz,Cf,YZfd),(Xz, Cf,YZfe),(Xz,Cf,YZff),(Xz,Cf,YZfg),(Xz,Cf,YZfh),(Xz,Cf,YZfi),(Xz,Cf,YZfj),(Xz, Cf,YZfk),(Xz,Cf,YZfl),(Xz,Cf,YZfm),(Xz,Cf,YZfn),(Xz,Cf,YZfo),(Xz,Cf,YZfp),(Xz, Cf,YZfq),(Xz,Cf,YZfr),(Xz,Cf,YZfs),(Xz,Cf,YZft),(Xz,Cf,YZfu),(Xz,Cf,YZfv),(Xz,Cf, YZfw),(Xz,Cf,YZfx),(Xz,Cf,YZfy),(Xz,Cf,YZfz),(Xz,Cf,YZga),(Xz,Cf,YZgb),(Xz,Cf, YZgc),(Xz,Cf,YZgd),(Xz,Cf,YZge),(Xz,Cf,YZgf),(Xz,Cf,YZgg),(Xz,Cf,YZgh),(Xz,Cf, YZgi),(Xz,Cf,YZgj),(Xz,Cf,YZgk),(Xz,Cf,YZgl),(Xz,Cf,YZgm),(Xz,Cf,YZgn),(Xz, Cf,YZgo),(Xz,Cf,YZgp),(Xz,Cf,YZgq),(Xz,Cf,YZgr),(Xz,Cf,YZgs),(Xz,Cf,YZgt), (Xaa,Ca,YZa),(Xaa,Ca,YZb),(Xaa,Ca,YZc),(Xaa,Ca,YZd),(Xaa,Ca,YZe),(Xaa,Ca,YZf), (Xaa,Ca,YZg),(Xaa,Ca,YZh),(Xaa,Ca,YZi),(Xaa,Ca,YZj),(Xaa,Ca,YZk),(Xaa,Ca,YZl), (Xaa,Ca,YZm),(Xaa,Ca,YZn),(Xaa,Ca,YZo),(Xaa,Ca,YZp),(Xaa,Ca,YZq),(Xaa,Ca, YZr),(Xaa,Ca,YZs),(Xaa,Ca,YZt),(Xaa,Ca,YZu),(Xaa,Ca,YZv),(Xaa,Ca,YZw),(Xaa,Ca, YZx),(Xaa,Ca,YZy),(Xaa,Ca,YZz),(Xaa,Ca,YZaa),(Xaa,Ca,YZab),(Xaa,Ca,YZac),(Xaa, Ca,YZad),(Xaa,Ca,YZae),(Xaa,Ca,YZaf),(Xaa,Ca,YZag),(Xaa,Ca,YZah),(Xaa,Ca, YZai),(Xaa,Ca,YZaj),(Xaa,Ca,YZak),(Xaa,Ca,YZal),(Xaa,Ca,YZam),(Xaa,Ca,YZan), (Xaa,Ca,YZao),(Xaa,Ca,YZap),(Xaa,Ca,YZaq),(Xaa,Ca,YZar),(Xaa,Ca,YZas),(Xaa,Ca, YZat),(Xaa,Ca,YZau),(Xaa,Ca,YZav),(Xaa,Ca,YZaw),(Xaa,Ca,YZax),(Xaa,Ca,YZay), (Xaa,Ca,YZaz),(Xaa,Ca,YZba),(Xaa,Ca,YZbb),(Xaa,Ca,YZbc),(Xaa,Ca,YZbd),(Xaa, Ca,YZbe),(Xaa,Ca,YZbf),(Xaa,Ca,YZbg),(Xaa,Ca,YZbh),(Xaa,Ca,YZbi),(Xaa,Ca,YZbj), (Xaa,Ca,YZbk),(Xaa,Ca,YZbl),(Xaa,Ca,YZbm),(Xaa,Ca,YZbn),(Xaa,Ca,YZbo),(Xaa, Ca,YZbp),(Xaa,Ca,YZbq),(Xaa,Ca,YZbr),(Xaa,Ca,YZbs),(Xaa,Ca,YZbt),(Xaa,Ca, YZbu),(Xaa,Ca,YZbv),(Xaa,Ca,YZbw),(Xaa,Ca,YZbx),(Xaa,Ca,YZby),(Xaa,Ca,YZbz), (Xaa,Ca,YZca),(Xaa,Ca,YZcb),(Xaa,Ca,YZcc),(Xaa,Ca,YZcd),(Xaa,Ca,YZce),(Xaa, Ca,YZcf),(Xaa,Ca,YZcg),(Xaa,Ca,YZch),(Xaa,Ca,YZci),(Xaa,Ca,YZcj),(Xaa,Ca,YZck), (Xaa,Ca,YZcl),(Xaa,Ca,YZcm),(Xaa,Ca,YZcn),(Xaa,Ca,YZco),(Xaa,Ca,YZcp),(Xaa, Ca,YZcq),(Xaa,Ca,YZcr),(Xaa,Ca,YZcs),(Xaa,Ca,YZct),(Xaa,Ca,YZcu),(Xaa,Ca,YZcv), (Xaa,Ca,YZcw),(Xaa,Ca,YZcx),(Xaa,Ca,YZcy),(Xaa,Ca,YZcz),(Xaa,Ca,YZda),(Xaa, Ca,YZdb),(Xaa,Ca,YZdc),(Xaa,Ca,YZdd),(Xaa,Ca,YZde),(Xaa,Ca,YZdf),(Xaa,Ca, YZdg),(Xaa,Ca,YZdh),(Xaa,Ca,YZdi),(Xaa,Ca,YZdj),(Xaa,Ca,YZdk),(Xaa,Ca,YZdl),(Xaa, Ca,YZdm),(Xaa,Ca,YZdn),(Xaa,Ca,YZdo),(Xaa,Ca,YZdp),(Xaa,Ca,YZdq),(Xaa, Ca,YZdr),(Xaa,Ca,YZds),(Xaa,Ca,YZdt),(Xaa,Ca,YZdu),(Xaa,Ca,YZdv),(Xaa,Ca,YZdw), (Xaa,Ca,YZdx),(Xaa,Ca,YZdy),(Xaa,Ca,YZdz),(Xaa,Ca,YZea),(Xaa,Ca,YZeb),(Xaa, Ca,YZec),(Xaa,Ca,YZed),(Xaa,Ca,YZee),(Xaa,Ca,YZef),(Xaa,Ca,YZeg),(Xaa,Ca,YZeh), (Xaa,Ca,YZei),(Xaa,Ca,YZej),(Xaa,Ca,YZek),(Xaa,Ca,YZel),(Xaa,Ca,YZem),(Xaa, Ca,YZen),(Xaa,Ca,YZeo),(Xaa,Ca,YZep),(Xaa,Ca,YZeq),(Xaa,Ca,YZer),(Xaa,Ca, YZes),(Xaa,Ca,YZet),(Xaa,Ca,YZeu),(Xaa,Ca,YZev),(Xaa,Ca,YZew),(Xaa,Ca,YZex), (Xaa,Ca,YZey),(Xaa,Ca,YZez),(Xaa,Ca,YZfa),(Xaa,Ca,YZfb),(Xaa,Ca,YZfc),(Xaa,Ca, YZfd),(Xaa,Ca,YZfe),(Xaa,Ca,YZff),(Xaa,Ca,YZfg),(Xaa,Ca,YZfh),(Xaa,Ca,YZfi),(Xaa, Ca,YZfj),(Xaa,Ca,YZfk),(Xaa,Ca,YZfl),(Xaa,Ca,YZfm),(Xaa,Ca,YZfn),(Xaa,Ca, YZfo),(Xaa,Ca,YZfp),(Xaa,Ca,YZfq),(Xaa,Ca,YZfr),(Xaa,Ca,YZfs),(Xaa,Ca,YZft),(Xaa, Ca,YZfu),(Xaa,Ca,YZfv),(Xaa,Ca,YZfw),(Xaa,Ca,YZfx),(Xaa,Ca,YZfy),(Xaa,Ca,YZfz), (Xaa,Ca,YZga),(Xaa,Ca,YZgb),(Xaa,Ca,YZgc),(Xaa,Ca,YZgd),(Xaa,Ca,YZge),(Xaa, Ca,YZgf),(Xaa,Ca,YZgg),(Xaa,Ca,YZgh),(Xaa,Ca,YZgi),(Xaa,Ca,YZgj),(Xaa,Ca, YZgk),(Xaa,Ca,YZgl),(Xaa,Ca,YZgm),(Xaa,Ca,YZgn),(Xaa,Ca,YZgo),(Xaa,Ca,YZgp), (Xaa,Ca,YZgq),(Xaa,Ca,YZgr),(Xaa,Ca,YZgs),(Xaa,Ca,YZgt),(Xaa,Cb,YZa),(Xaa,Cb, YZb),(Xaa,Cb,YZc),(Xaa,Cb,YZd),(Xaa,Cb,YZe),(Xaa,Cb,YZf),(Xaa,Cb,YZg),(Xaa, Cb,YZh),(Xaa,Cb,YZi),(Xaa,Cb,YZj),(Xaa,Cb,YZk),(Xaa,Cb,YZl),(Xaa,Cb,YZm),(Xaa, Cb,YZn),(Xaa,Cb,YZo),(Xaa,Cb,YZp),(Xaa,Cb,YZq),(Xaa,Cb,YZr),(Xaa,Cb,YZs), (Xaa,Cb,YZt),(Xaa,Cb,YZu),(Xaa,Cb,YZv),(Xaa,Cb,YZw),(Xaa,Cb,YZx),(Xaa,Cb,YZy), (Xaa,Cb,YZz),(Xaa,Cb,YZaa),(Xaa,Cb,YZab),(Xaa,Cb,YZac),(Xaa,Cb,YZad),(Xaa, Cb,YZae),(Xaa,Cb,YZaf),(Xaa,Cb,YZag),(Xaa,Cb,YZah),(Xaa,Cb,YZai),(Xaa,Cb,YZaj), (Xaa,Cb,YZak),(Xaa,Cb,YZal),(Xaa,Cb,YZam),(Xaa,Cb,YZan),(Xaa,Cb,YZao),(Xaa, Cb,YZap),(Xaa,Cb,YZaq),(Xaa,Cb,YZar),(Xaa,Cb,YZas),(Xaa,Cb,YZat),(Xaa,Cb, YZau),(Xaa,Cb,YZav),(Xaa,Cb,YZaw),(Xaa,Cb,YZax),(Xaa,Cb,YZay),(Xaa,Cb,YZaz), (Xaa,Cb,YZba),(Xaa,Cb,YZbb),(Xaa,Cb,YZbc),(Xaa,Cb,YZbd),(Xaa,Cb,YZbe),(Xaa, Cb,YZbf),(Xaa,Cb,YZbg),(Xaa,Cb,YZbh),(Xaa,Cb,YZbi),(Xaa,Cb,YZbj),(Xaa,Cb,YZbk), (Xaa,Cb,YZbl),(Xaa,Cb,YZbm),(Xaa,Cb,YZbn),(Xaa,Cb,YZbo),(Xaa,Cb,YZbp), (Xaa,Cb,YZbq),(Xaa,Cb,YZbr),(Xaa,Cb,YZbs),(Xaa,Cb,YZbt),(Xaa,Cb,YZbu),(Xaa,Cb, YZbv),(Xaa,Cb,YZbw),(Xaa,Cb,YZbx),(Xaa,Cb,YZby),(Xaa,Cb,YZbz),(Xaa,Cb,YZca), (Xaa,Cb,YZcb),(Xaa,Cb,YZcc),(Xaa,Cb,YZcd),(Xaa,Cb,YZce),(Xaa,Cb,YZcf),(Xaa, Cb,YZcg),(Xaa,Cb,YZch),(Xaa,Cb,YZci),(Xaa,Cb,YZcj),(Xaa,Cb,YZck),(Xaa,Cb, YZcl),(Xaa,Cb,YZcm),(Xaa,Cb,YZcn),(Xaa,Cb,YZco),(Xaa,Cb,YZcp),(Xaa,Cb,YZcq), (Xaa,Cb,YZcr),(Xaa,Cb,YZcs),(Xaa,Cb,YZct),(Xaa,Cb,YZcu),(Xaa,Cb,YZcv),(Xaa, Cb,YZcw),(Xaa,Cb,YZcx),(Xaa,Cb,YZcy),(Xaa,Cb,YZcz),(Xaa,Cb,YZda),(Xaa,Cb,YZdb), (Xaa,Cb,YZdc),(Xaa,Cb,YZdd),(Xaa,Cb,YZde),(Xaa,Cb,YZdf),(Xaa,Cb,YZdg),(Xaa, Cb,YZdh),(Xaa,Cb,YZdi),(Xaa,Cb,YZdj),(Xaa,Cb,YZdk),(Xaa,Cb,YZdl),(Xaa,Cb, YZdm),(Xaa,Cb,YZdn),(Xaa,Cb,YZdo),(Xaa,Cb,YZdp),(Xaa,Cb,YZdq),(Xaa,Cb,YZdr), (Xaa,Cb,YZds),(Xaa,Cb,YZdt),(Xaa,Cb,YZdu),(Xaa,Cb,YZdv),(Xaa,Cb,YZdw),(Xaa, Cb,YZdx),(Xaa,Cb,YZdy),(Xaa,Cb,YZdz),(Xaa,Cb,YZea),(Xaa,Cb,YZeb),(Xaa,Cb, YZec),(Xaa,Cb,YZed),(Xaa,Cb,YZee),(Xaa,Cb,YZef),(Xaa,Cb,YZeg),(Xaa,Cb,YZeh), (Xaa,Cb,YZei),(Xaa,Cb,YZej),(Xaa,Cb,YZek),(Xaa,Cb,YZel),(Xaa,Cb,YZem),(Xaa,Cb, YZen),(Xaa,Cb,YZeo),(Xaa,Cb,YZep),(Xaa,Cb,YZeq),(Xaa,Cb,YZer),(Xaa,Cb,YZes), (Xaa,Cb,YZet),(Xaa,Cb,YZeu),(Xaa,Cb,YZev),(Xaa,Cb,YZew),(Xaa,Cb,YZex),(Xaa, Cb,YZey),(Xaa,Cb,YZez),(Xaa,Cb,YZfa),(Xaa,Cb,YZfb),(Xaa,Cb,YZfc),(Xaa,Cb,YZfd), (Xaa,Cb,YZfe),(Xaa,Cb,YZff),(Xaa,Cb,YZfg),(Xaa,Cb,YZfh),(Xaa,Cb,YZfi),(Xaa, Cb,YZfj),(Xaa,Cb,YZfk),(Xaa,Cb,YZfl),(Xaa,Cb,YZfm),(Xaa,Cb,YZfn),(Xaa,Cb,YZfo), (Xaa,Cb,YZfp),(Xaa,Cb,YZfq),(Xaa,Cb,YZfr),(Xaa,Cb,YZfs),(Xaa,Cb,YZft),(Xaa, Cb,YZfu),(Xaa,Cb,YZfv),(Xaa,Cb,YZfw),(Xaa,Cb,YZfx),(Xaa,Cb,YZfy),(Xaa,Cb,YZfz), (Xaa,Cb,YZga),(Xaa,Cb,YZgb),(Xaa,Cb,YZgc),(Xaa,Cb,YZgd),(Xaa,Cb,YZge),(Xaa, Cb,YZgf),(Xaa,Cb,YZgg),(Xaa,Cb,YZgh),(Xaa,Cb,YZgi),(Xaa,Cb,YZgj),(Xaa,Cb, YZgk),(Xaa,Cb,YZgl),(Xaa,Cb,YZgm),(Xaa,Cb,YZgn),(Xaa,Cb,YZgo),(Xaa,Cb,YZgp), (Xaa,Cb,YZgq),(Xaa,Cb,YZgr),(Xaa,Cb,YZgs),(Xaa,Cb,YZgt),(Xaa,Cc,YZa),(Xaa, Cc,YZb),(Xaa,Cc,YZc),(Xaa,Cc,YZd),(Xaa,Cc,YZe),(Xaa,Cc,YZf),(Xaa,Cc,YZg),(Xaa, Cc,YZh),(Xaa,Cc,YZi),(Xaa,Cc,YZj),(Xaa,Cc,YZk),(Xaa,Cc,YZl),(Xaa,Cc,YZm), (Xaa,Cc,YZn),(Xaa,Cc,YZo),(Xaa,Cc,YZp),(Xaa,Cc,YZq),(Xaa,Cc,YZr),(Xaa,Cc,YZs), (Xaa,Cc,YZt),(Xaa,Ce,YZu),(Xaa,Cc,YZv),(Xaa,Cc,YZw),(Xaa,Cc,YZx),(Xaa,Cc, YZy),(Xaa,Cc,YZz),(Xaa,Cc,YZaa),(Xaa,Cc,YZab),(Xaa,Cc,YZac),(Xaa,Cc,YZad),(Xaa, Cc,YZae),(Xaa,Cc,YZaf),(Xaa,Cc,YZag),(Xaa,Cc,YZah),(Xaa,Cc,YZai),(Xaa,Cc,YZaj), (Xaa,Cc,YZak),(Xaa,Cc,YZal),(Xaa,Cc,YZam),(Xaa,Cc,YZan),(Xaa,Cc,YZao),(Xaa, Cc,YZap),(Xaa,Cc,YZaq),(Xaa,Cc,YZar),(Xaa,Cc,YZas),(Xaa,Cc,YZat),(Xaa,Cc, YZau),(Xaa,Cc,YZav),(Xaa,Cc,YZaw),(Xaa,Cc,YZax),(Xaa,Cc,YZay),(Xaa,Cc,YZaz), (Xaa,Cc,YZba),(Xaa,Cc,YZbb),(Xaa,Cc,YZbc),(Xaa,Cc,YZbd),(Xaa,Cc,YZbe),(Xaa, Ce,YZbf),(Xaa,Cc,YZbg),(Xaa,Cc,YZbh),(Xaa,Cc,YZbi),(Xaa,Cc,YZbj),(Xaa,Cc,YZbk), (Xaa,Cc,YZbl),(Xaa,Cc,YZbm),(Xaa,Cc,YZbn),(Xaa,Cc,YZbo),(Xaa,Cc,YZbp),(Xaa, Cc,YZbq),(Xaa,Cc,YZbr),(Xaa,Cc,YZbs),(Xaa,Cc,YZbt),(Xaa,Cc,YZbu),(Xaa,Cc,YZbv), (Xaa,Cc,YZbw),(Xaa,Cc,YZbx),(Xaa,Cc,YZby),(Xaa,Cc,YZbz),(Xaa,Cc,YZca),(Xaa, Cc,YZcb),(Xaa,Cc,YZcc),(Xaa,Cc,YZcd),(Xaa,Cc,YZce),(Xaa,Cc,YZcf),(Xaa,Cc, YZcg),(Xaa,Cc,YZch),(Xaa,Cc,YZci),(Xaa,Cc,YZcj),(Xaa,Cc,YZck),(Xaa,Cc,YZcl),(Xaa, Cc,YZcm),(Xaa,Cc,YZcn),(Xaa,Cc,YZco),(Xaa,Cc,YZcp),(Xaa,Cc,YZcq),(Xaa,Cc, YZcr),(Xaa,Cc,YZcs),(Xaa,Cc,YZct),(Xaa,Cc,YZcu),(Xaa,Cc,YZcv),(Xaa,Cc,YZcw), (Xaa,Cc,YZcx),(Xaa,Cc,YZcy),(Xaa,Cc,YZcz),(Xaa,Cc,YZda),(Xaa,Cc,YZdb),(Xaa, Cc,YZdc),(Xaa,Cc,YZdd),(Xaa,Cc,YZde),(Xaa,Cc,YZdf),(Xaa,Cc,YZdg),(Xaa,Cc,YZdh), (Xaa,Cc,YZdi),(Xaa,Cc,YZdj),(Xaa,Cc,YZdk),(Xaa,Cc,YZdl),(Xaa,Cc,YZdm),(Xaa, Cc,YZdn),(Xaa,Cc,YZdo),(Xaa,Cc,YZdp),(Xaa,Cc,YZdq),(Xaa,Cc,YZdr),(Xaa,Cc, YZds),(Xaa,Cc,YZdt),(Xaa,Cc,YZdu),(Xaa,Cc,YZdv),(Xaa,Cc,YZdw),(Xaa,Cc,YZdx), (Xaa,Cc,YZdy),(Xaa,Cc,YZdz),(Xaa,Cc,YZea),(Xaa,Cc,YZeb),(Xaa,Cc,YZec),(Xaa,Cc, YZed),(Xaa,Cc,YZee),(Xaa,Cc,YZef),(Xaa,Cc,YZeg),(Xaa,Cc,YZeh),(Xaa,Cc,YZei), (Xaa,Cc,YZej),(Xaa,Cc,YZek),(Xaa,Cc,YZel),(Xaa,Cc,YZem),(Xaa,Cc,YZen),(Xaa, Cc,YZeo),(Xaa,Cc,YZep),(Xaa,Cc,YZeq),(Xaa,Cc,YZer),(Xaa,Cc,YZes),(Xaa,Cc,YZet), (Xaa,Cc,YZeu),(Xaa,Cc,YZev),(Xaa,Cc,YZew),(Xaa,Cc,YZex),(Xaa,Cc,YZey),(Xaa, Cc,YZez),(Xaa,Cc,YZfa),(Xaa,Cc,YZfb),(Xaa,Cc,YZfc),(Xaa,Cc,YZfd),(Xaa,Cc,YZfe), (Xaa,Cc,YZff),(Xaa,Cc,YZfg),(Xaa,Cc,YZfh),(Xaa,Cc,YZfi),(Xaa,Cc,YZfj),(Xaa, Cc,YZfk),(Xaa.Cc,YZfl),(Xaa,Cc,YZfm),(Xaa,Cc,YZfn),(Xaa,Cc,YZfo),(Xaa,Cc,YZfp), (Xaa,Cc,YZfq),(Xaa,Cc,YZfr),(Xaa,Cc,YZfs),(Xaa,Cc,YZft),(Xaa,Cc,YZfu),(Xaa,Cc, YZfv),(Xaa,Cc,YZfw),(Xaa,Cc,YZfx),(Xaa,Cc,YZfy),(Xaa,Cc,YZfz),(Xaa,Cc,YZga), (Xaa,Cc,YZgb),(Xaa,Cc,YZgc),(Xaa,Cc,YZgd),(Xaa,Cc,YZge),(Xaa,Cc,YZgf),(Xaa, Cc,YZgg),(Xaa,Cc,YZgh),(Xaa,Cc,YZgi),(Xaa,Cc,YZgj),(Xaa,Cc,YZgk),(Xaa,Cc,YZgl), (Xaa,Cc,YZgm),(Xaa,Cc,YZgn),(Xaa,Cc,YZgo),(Xaa,Cc,YZgp),(Xaa,Cc,YZgq),(Xaa, Cc,YZgr),(Xaa,Cc,YZgs),(Xaa,Cc,YZgt),(Xaa,Cd,YZa),(Xaa,Cd,YZb),(Xaa,Cd,YZc), (Xaa,Cd,YZd),(Xaa,Cd,YZe),(Xaa,Cd,YZf),(Xaa,Cd,YZg),(Xaa,Cd,YZh),(Xaa,Cd, YZi),(Xaa,Cd,YZj),(Xaa,Cd,YZk),(Xaa,Cd,YZl),(Xaa,Cd,YZm),(Xaa,Cd,YZn),(Xaa, Cd,YZo),(Xaa,Cd,YZp),(Xaa,Cd,YZq),(Xaa,Cd,YZr),(Xaa,Cd,YZs),(Xaa,Cd,YZt),(Xaa, Cd,YZu),(Xaa,Cd,YZv),(Xaa,Cd,YZw),(Xaa,Cd,YZx),(Xaa,Cd,YZy),(Xaa,Cd,YZz), (Xaa,Cd,YZaa),(Xaa,Cd,YZab),(Xaa,Cd,YZac),(Xaa,Cd,YZad),(Xaa,Cd,YZae),(Xaa, Cd,YZaf),(Xaa,Cd,YZag),(Xaa,Cd,YZah),(Xaa,Cd,YZai),(Xaa,Cd,YZaj),(Xaa,Cd,YZak), (Xaa,Cd,YZal),(Xaa,Cd,YZam),(Xaa,Cd,YZan),(Xaa,Cd,YZao),(Xaa,Cd,YZap),(Xaa, Cd,YZaq),(Xaa,Cd,YZar),(Xaa,Cd,YZas),(Xaa,Cd,YZat),(Xaa,Cd,YZau),(Xaa,Cd,YZav), (Xaa,Cd,YZaw),(Xaa,Cd,YZax),(Xaa,Cd,YZay),(Xaa,Cd,YZaz),(Xaa,Cd,YZba),(Xaa, Cd,YZbb),(Xaa,Cd,YZbc),(Xaa,Cd,YZbd),(Xaa,Cd,YZbe),(Xaa,Cd,YZbf),(Xaa,Cd, YZbg),(Xaa,Cd,YZbh),(Xaa,Cd,YZbi),(Xaa,Cd,YZbj),(Xaa,Cd,YZbk),(Xaa,Cd,YZbl), (Xaa,Cd,YZbm),(Xaa,Cd,YZbn),(Xaa,Cd,YZbo),(Xaa,Cd,YZbp),(Xaa,Cd,YZbq),(Xaa, Cd,YZbr),(Xaa,Cd,YZbs),(Xaa,Cd,YZbt),(Xaa,Cd,YZbu),(Xaa,Cd,YZbv),(Xaa,Cd, YZbw),(Xaa,Cd,YZbx),(Xaa,Cd,YZby),(Xaa,Cd,YZbz),(Xaa,Cd,YZca),(Xaa,Cd,YZcb), (Xaa,Cd,YZcc),(Xaa,Cd,YZcd),(Xaa,Cd,YZce),(Xaa,Cd,YZcf),(Xaa,Cd,YZcg),(Xaa, Cd,YZch),(Xaa,Cd,YZci),(Xaa,Cd,YZcj),(Xaa,Cd,YZck),(Xaa,Cd,YZcl),(Xaa,Cd,YZcm), (Xaa,Cd,YZcn),(Xaa,Cd,YZco),(Xaa,Cd,YZcp),(Xaa,Cd,YZcq),(Xaa,Cd,YZcr),(Xaa, Cd,YZcs),(Xaa,Cd,YZct),(Xaa,Cd,YZcu),(Xaa,Cd,YZcv),(Xaa,Cd,YZcw),(Xaa,Cd, YZcx),(Xaa,Cd,YZcy),(Xaa,Cd,YZcz),(Xaa,Cd,YZda),(Xaa,Cd,YZdb),(Xaa,Cd,YZdc), (Xaa,Cd,YZdd),(Xaa,Cd,YZde),(Xaa,Cd,YZdf),(Xaa,Cd,YZdg),(Xaa,Cd,YZdh),(Xaa, Cd,YZdi),(Xaa,Cd,YZdj),(Xaa,Cd,YZdk),(Xaa,Cd,YZdl),(Xaa,Cd,YZdm),(Xaa,Cd,YZdn), (Xaa,Cd,YZdo),(Xaa,Cd,YZdp),(Xaa,Cd,YZdq),(Xaa,Cd,YZdr),(Xaa,Cd,YZds),(Xaa, Cd,YZdt),(Xaa,Cd,YZdu),(Xaa,Cd,YZdv),(Xaa,Cd,YZdw),(Xaa,Cd,YZdx),(Xaa,Cd, YZdy),(Xaa,Cd,YZdz),(Xaa,Cd,YZea),(Xaa,Cd,YZeb),(Xaa,Cd,YZec),(Xaa,Cd,YZed), (Xaa,Cd,YZee),(Xaa,Cd,YZef),(Xaa,Cd,YZeg),(Xaa,Cd,YZeh),(Xaa,Cd,YZei),(Xaa, Cd,YZej),(Xaa,Cd,YZek),(Xaa,Cd,YZel),(Xaa,Cd,YZem),(Xaa,Cd,YZen),(Xaa,Cd,YZeo), (Xaa,Cd,YZep),(Xaa,Cd,YZeq),(Xaa,Cd,YZer),(Xaa,Cd,YZes),(Xaa,Cd,YZet),(Xaa, Cd,YZeu),(Xaa,Cd,YZev),(Xaa,Cd,YZew),(Xaa,Cd,YZex),(Xaa,Cd,YZey),(Xaa,Cd, YZez),(Xaa,Cd,YZfa),(Xaa,Cd,YZfb),(Xaa,Cd,YZfc),(Xaa,Cd,YZfd),(Xaa,Cd,YZfe),(Xaa, Cd,YZff),(Xaa,Cd,YZfg),(Xaa,Cd,YZfh),(Xaa,Cd,YZfi),(Xaa,Cd,YZfi),(Xaa,Cd, YZfk),(Xaa,Cd,YZfl),(Xaa,Cd,YZfm),(Xaa,Cd,YZfn),(Xaa,Cd,YZfo),(Xaa,Cd,YZfp),(Xaa, Cd,YZfq),(Xaa,Cd,YZfr),(Xaa,Cd,YZfs),(Xaa,Cd,YZft),(Xaa,Cd,YZfu),(Xaa,Cd, YZfv),(Xaa,Cd,YZfw),(Xaa,Cd,YZfx),(Xaa,Cd,YZfy),(Xaa,Cd,YZfz),(Xaa,Cd,YZga), (Xaa,Cd,YZgb),(Xaa,Cd,YZgc),(Xaa,Cd,YZgd),(Xaa,Cd,YZge),(Xaa,Cd,YZgf),(Xaa, Cd,YZgg),(Xaa,Cd,YZgh),(Xaa,Cd,YZgi),(Xaa,Cd,YZgj),(Xaa,Cd,YZgk),(Xaa,Cd,YZgl), (Xaa,Cd,YZgm),(Xaa,Cd,YZgn),(Xaa,Cd,YZgo),(Xaa,Cd,YZgp),(Xaa,Cd,YZgq),(Xaa, Cd,YZgr),(Xaa,Cd,YZgs),(Xaa,Cd,YZgt),(Xaa,Ce,YZa),(Xaa,Ce,YZb),(Xaa,Ce,YZc), (Xaa,Ce,YZd),(Xaa,Ce,YZe),(Xaa,Ce,YZf),(Xaa,Ce,YZg),(Xaa,Ce,YZh),(Xaa,Ce, YZi),(Xaa,Ce,YZj),(Xaa,Ce,YZk),(Xaa,Ce,YZl),(Xaa,Ce,YZm),(Xaa,Ce,YZn),(Xaa,Ce, YZo),(Xaa,Ce,YZp),(Xaa,Ce,YZq),(Xaa,Ce,YZr),(Xaa,Ce,YZs),(Xaa,Ce,YZt),(Xaa, Ce,YZu),(Xaa,Ce,YZv),(Xaa,Ce,YZw),(Xaa,Ce,YZx),(Xaa,Ce,YZy),(Xaa,Ce,YZz),(Xaa, Ce,YZaa),(Xaa,Ce,YZab),(Xaa,Ce,YZac),(Xaa,Ce,YZad),(Xaa,Ce,YZae),(Xaa,Ce, YZaf),(Xaa,Ce,YZag),(Xaa,Ce,YZah),(Xaa,Ce,YZai),(Xaa,Ce,YZaj),(Xaa,Ce,YZak),(Xaa, Ce,YZal),(Xaa,Ce,YZam),(Xaa,Ce,YZan),(Xaa,Ce,YZao),(Xaa,Ce,YZap),(Xaa,Ce, YZaq),(Xaa,Ce,YZar),(Xaa,Ce,YZas),(Xaa,Ce,YZat),(Xaa,Ce,YZau),(Xaa,Ce,YZav), (Xaa,Ce,YZaw),(Xaa,Ce,YZax),(Xaa,Ce,YZay),(Xaa,Ce,YZaz),(Xaa,Ce,YZba),(Xaa, Ce,YZbb),(Xaa,Ce,YZbc),(Xaa,Ce,YZbd),(Xaa,Ce,YZbe),(Xaa,Ce,YZbf),(Xaa,Ce,YZbg), (Xaa,Ce,YZbh),(Xaa,Ce,YZbi),(Xaa,Ce,YZbj),(Xaa,Ce,YZbk),(Xaa,Ce,YZbl),(Xaa, Ce,YZbm),(Xaa,Ce,YZbn),(Xaa,Ce,YZbo),(Xaa,Ce,YZbp),(Xaa,Ce,YZbq),(Xaa,Ce, YZbr),(Xaa,Ce,YZbs),(Xaa,Ce,YZbt),(Xaa,Ce,YZbu),(Xaa,Ce,YZbv),(Xaa,Ce,YZbw), (Xaa,Ce,YZbx),(Xaa,Ce,YZby),(Xaa,Ce,YZbz),(Xaa,Ce,YZca),(Xaa,CC,YZcb),(Xaa,Ce, YZcc),(Xaa,Ce,YZcd),(Xaa,Ce,YZce),(Xaa,Ce,YZcf),(Xaa,Ce,YZcg),(Xaa,Ce,YZch), (Xaa,Ce,YZci),(Xaa,Ce,YZcj),(Xaa,Ce,YZck),(Xaa,Ce,YZcl),(Xaa,Ce,YZcm),(Xaa, Ce,YZcn),(Xaa,Ce,YZco),(Xaa,Ce,YZcp),(Xaa,Ce,YZcq),(Xaa,Ce,YZcr),(Xaa,Ce,YZcs), (Xaa,Ce,YZct),(Xaa,Ce,YZcu),(Xaa,Ce,YZcv),(Xaa,Ce,YZcw),(Xaa,Ce,YZcx),(Xaa, Cc,YZey),(Xaa,Ce,YZez),(Xaa,Ce,YZda),(Xaa,Ce,YZdb),(Xaa,Ce,YZdc),(Xaa,Ce,YZdd), (Xaa,Ce,YZde),(Xaa,Ce,YZdf),(Xaa,Ce,YZdg),(Xaa,Ce,YZdh),(Xaa,CC,YZdi),(Xaa, Ce,YZdj),(Xaa,Ce,YZdk),(Xaa,Ce,YZdl),(Xaa,Ce,YZdm),(Xaa,Ce,YZdn),(Xaa,Ce, YZdo),(Xaa,Ce,YZdp),(Xaa,Ce,YZdq),(Xaa,Ce,YZdr),(Xaa,Ce,YZds),(Xaa,Ce,YZdt), (Xaa,Ce,YZdu),(Xaa,Ce,YZdv),(Xaa,Ce,YZdw),(Xaa,Ce,YZdx),(Xaa,Ce,YZdy),(Xaa, Ce,YZdz),(Xaa,Ce,YZea),(Xaa,Ce,YZeb),(Xaa,Cc,YZec),(Xaa,Ce,YZed),(Xaa,Ce,YZee), (Xaa,Ce,YZef),(Xaa,Ce,YZeg),(Xaa,Ce,YZeh),(Xaa,Ce,YZei),(Xaa,Ce,YZej),(Xaa, Ce,YZek),(Xaa,Ce,YZel),(Xaa,Ce,YZem),(Xaa,Ce,YZen),(Xaa,Ce,YZeo),(Xaa,Ce, YZep),(Xaa,Ce,YZeq),(Xaa,Ce,YZer),(Xaa,Ce,YZes),(Xaa,Ce,YZet),(Xaa,Ce,YZeu),(Xaa, Ce,YZev),(Xaa,Ce,YZew),(Xaa,Ce,YZex),(Xaa,Ce,YZey),(Xaa,Ce,YZez),(Xaa,Ce, YZfa),(Xaa,Cc,YZfb),(Xaa,Ce,YZfc),(Xaa,Ce,YZfd),(Xaa,CC,YZfe),(Xaa,Ce,YZff),(Xaa, Ce,YZfg),(Xaa,Ce,YZfh),(Xaa,Ce,YZfi),(Xaa,Ce,YZfj),(Xaa,Ce,YZfk),(Xaa,Ce, YZfl),(Xaa,Ce,YZfm),(Xaa,Ce,YZfn),(Xaa,Ce,YZfo),(Xaa,Ce,YZfp),(Xaa,Ce,YZfq),(Xaa, Ce,YZfr),(Xaa,Ce,YZfs),(Xaa,Ce,YZft),(Xaa,Ce,YZfu),(Xaa,Ce,YZfv),(Xaa,Ce,YZfw), (Xaa,Ce,YZfx),(Xaa,Ce,YZfy),(Xaa,Ce,YZfz),(Xaa,Ce,YZga),(Xaa,Ce,YZgb),(Xaa, Ce,YZgc),(Xaa,Ce,YZgd),(Xaa,Ce,YZge),(Xaa,Ce,YZgf),(Xaa,Ce,YZgg),(Xaa,Ce, YZgh),(Xaa,Ce,YZgi),(Xaa,Ce,YZgj),(Xaa,Ce,YZgk),(Xaa,Ce,YZgl),(Xaa,Ce,YZgm), (Xaa,Ce,YZgn),(Xaa,Ce,YZgo),(Xaa,Ce,YZgp),(Xaa,Ce,YZgq),(Xaa,Ce,YZgr),(Xaa, Ce,YZgs),(Xaa,Ce,YZgt),(Xaa,Cf,YZa),(Xaa,Cf,YZb),(Xaa,Cf,YZc),(Xaa,Cf,YZd),(Xaa, Cf,YZe),(Xaa,Cf,YZf),(Xaa,Cf,YZg),(Xaa,Cf,YZh),(Xaa,Cf,YZi),(Xaa,Cf,YZj),(Xaa, Cf,YZk),(Xaa,Cf,YZl),(Xaa,Cf,YZm),(Xaa,Cf,YZn),(Xaa,Cf,YZo),(Xaa,Cf,YZp),(Xaa, Cf,YZq),(Xaa,Cf,YZr),(Xaa,Cf,YZs),(Xaa,Cf,YZt),(Xaa,Cf,YZu),(Xaa,Cf,YZv),(Xaa, Cf,YZw),(Xaa,Cf,YZx),(Xaa,Cf,YZy),(Xaa,Cf,YZz),(Xaa,Cf,YZaa),(Xaa,Cf,YZab), (Xaa,Cf,YZac),(Xaa,Cf,YZad),(Xaa,Cf,YZae),(Xaa,Cf,YZaf),(Xaa,Cf,YZag),(Xaa,Cf, YZah),(Xaa,Cf,YZai),(Xaa,Cf,YZaj),(Xaa,Cf,YZak),(Xaa,Cf,YZal),(Xaa,Cf,YZam),(Xaa, Cf,YZan),(Xaa,Cf,YZao),(Xaa,Cf,YZap),(Xaa,Cf,YZaq),(Xaa,Cf,YZar),(Xaa,Cf, YZas),(Xaa,Cf,YZat),(Xaa,Cf,YZau),(Xaa,Cf,YZav),(Xaa,Cf,YZaw),(Xaa,Cf,YZax),(Xaa, Cf,YZay),(Xaa,Cf,YZaz),(Xaa,Cf,YZba),(Xaa,Cf,YZbb),(Xaa,Cf,YZbc),(Xaa,Cf, YZbd),(Xaa,Cf,YZbe),(Xaa,Cf,YZbf),(Xaa,Cf,YZbg),(Xaa,Cf,YZbh),(Xaa,Cf,YZbi),(Xaa, Cf,YZbj),(Xaa,Cf,YZbk),(Xaa,Cf,YZbl),(Xaa,Cf,YZbm),(Xaa,Cf,YZbn),(Xaa,Cf,YZbo), (Xaa,Cf,YZbp),(Xaa,Cf,YZbq),(Xaa,Cf,YZbr),(Xaa,Cf,YZbs),(Xaa,Cf,YZbt),(Xaa, Cf,YZbu),(Xaa,Cf,YZbv),(Xaa,Cf,YZbw),(Xaa,Cf,YZbx),(Xaa,Cf,YZby),(Xaa,Cf, YZbz),(Xaa,Cf,YZca),(Xaa,Cf,YZcb),(Xaa,Cf,YZcc),(Xaa,Cf,YZcd),(Xaa,Cf,YZce),(Xaa, Cf,YZcf),(Xaa,Cf,YZcg),(Xaa,Cf,YZch),(Xaa,Cf,YZci),(Xaa,Cf,YZcj),(Xaa,Cf,YZck), (Xaa,Cf,YZcl),(Xaa,Cf,YZcm),(Xaa,Cf,YZcn),(Xaa,Cf,YZco),(Xaa,Cf,YZcp),(Xaa, Cf,YZcq),(Xaa,Cf,YZcr),(Xaa,Cf,YZcs),(Xaa,Cf,YZct),(Xaa,Cf,YZcu),(Xaa,Cf,YZcv), (Xaa,Cf,YZcw),(Xaa,Cf,YZcx),(Xaa,Cf,YZcy),(Xaa,Cf,YZcz),(Xaa,Cf,YZda),(Xaa, Cf,YZdb),(Xaa,Cf,YZdc),(Xaa,Cf,YZdd),(Xaa,Cf,YZde),(Xaa,Cf,YZdf),(Xaa,Cf,YZdg), (Xaa,Cf,YZdh),(Xaa,Cf,YZdi),(Xaa,Cf,YZdj),(Xaa,Cf,YZdk),(Xaa,Cf,YZdl),(Xaa, Cf,YZdm),(Xaa,Cf,YZdn),(Xaa,Cf,YZdo),(Xaa,Cf,YZdp),(Xaa,Cf,YZdq),(Xaa,Cf,YZdr), (Xaa,Cf,YZds),(Xaa,Cf,YZdt),(Xaa,Cf,YZdu),(Xaa,Cf,YZdv),(Xaa,Cf,YZdw),(Xaa, Cf,YZdx),(Xaa,Cf,YZdy),(Xaa,Cf,YZdz),(Xaa,Cf,YZea),(Xaa,Cf,YZeb),(Xaa,Cf,YZec), (Xaa,Cf,YZed),(Xaa,Cf,YZee),(Xaa,Cf,YZef),(Xaa,Cf,YZeg),(Xaa,Cf,YZeh),(Xaa, Cf,YZei),(Xaa,Cf,YZej),(Xaa,Cf,YZek),(Xaa,Cf,YZel),(Xaa,Cf,YZem),(Xaa,Cf,YZen), (Xaa,Cf,YZeo),(Xaa,Cf,YZep),(Xaa,Cf,YZeq),(Xaa,Cf,YZer),(Xaa,Cf,YZes),(Xaa,Cf, YZet),(Xaa,Cf,YZeu),(Xaa,Cf,YZev),(Xaa,Cf,YZew),(Xaa,Cf,YZex),(Xaa,Cf,YZey),(Xaa, Cf,YZez),(Xaa,Cf,YZfa),(Xaa,Cf,YZfb),(Xaa,Cf,YZfc),(Xaa,Cf,YZfd),(Xcaa,Cf,VZfe), (Xaa,Cf,YZff),(Xaa,Cf,YZfg),(Xaa,Cf,YZfh),(Xaa,Cf,YZfi),(Xaa,Cf,YZfj),(Xaa, Cf,YZfk),(Xaa,Cf,YZfl),(Xaa,Cf,YZfm),(Xaa,Cf,YZfn),(Xaa,Cf,YZfo),(Xaa,Cf,YZfp), (Xaa,Cf,YZfq),(Xaa,Cf,YZfr),(Xaa,Cf,YZfs),(Xaa,Cf,YZft),(Xaa,Cf,YZfu),(Xaa,Cf,YZfv), (Xaa,Cf,YZfw),(Xaa,Cf,YZfx),(Xaa,Cf,YZfy),(Xaa,Cf,YZfz),(Xaa,Cf,YZga),(Xaa, Cf,YZgb),(Xaa,Cf,YZgc),(Xaa,Cf,YZgd),(Xaa,Cf,YZge),(Xaa,Cf,YZgf),(Xaa,Cf,YZgg), (Xaa,Cf,YZgh),(Xaa,Cf,YZgi),(Xaa,Cf,YZgj),(Xaa,Cf,YZgk),(Xaa,Cf,YZgl),(Xaa, Cf,YZgm),(Xaa,Cf,YZgn),(Xaa,Cf,YZgo),(Xaa,Cf,YZgp),(Xaa,Cf,YZgq),(Xaa,Cf,YZgr), (Xaa,Cf,YZgs),(Xaa,Cf,YZgt), (Xab,Ca,YZa),(Xab,Ca,YZb),(Xab,Ca,YZc),(Xab,Ca,YZd),(Xab,Ca,YZe),(Xab,Ca,YZf), (Xab,Ca,YZg),(Xab,,Ca,YZh),(Xab,Ca,YZi),(Xab,Ca,YZj),(Xab,Ca,YZk),(Xab,Ca,YZl), (Xab,Ca,YZm),(Xab,Ca,YZn),(Xab,Ca,YZo),(Xab,Ca,YZp),(Xab,Ca,YZq),(Xab,Ca, YZr),(Xab,Ca,YZs),(Xab,Ca,YZt),(Xab,Ca,YZu),(Xab,Ca,YZv),(Xab,Ca,YZw),(Xab, Ca,YZx),(Xab,Ca,YZy),(Xab,Ca,YZz),(Xab,Ca,YZaa),(Xab,Ca,YZab),(Xab,Ca,YZac), (Xab,Ca,YZad),(Xab,Ca,YZae),(Xab,Ca,YZaf),(Xab,Ca,YZag),(Xab,Ca,YZah),(Xab, Ca,YZai),(Xab,Ca,YZaj),(Xab,Ca,YZak),(Xab,Ca,YZal),(Xab,Ca,YZam),(Xab,Ca,YZan), (Xab,Ca,YZao),(Xab,Ca,YZap),(Xab,Ca,YZaq),(Xab,Ca,YZar),(Xab,Ca,YZas),(Xab, Ca,YZat),(Xab,Ca,YZau),(Xab,Ca,YZav),(Xab,Ca,YZaw),(Xab,Ca,YZax),(Xab,Ca, YZay),(Xab,Ca,YZaz),(Xab,Ca,YZba),(Xab,Ca,YZbb),(Xab,Ca,YZbc),(Xab,Ca,YZbd), (Xab,Ca,YZbe),(Xab,Ca,YZbf),(Xab,Ca,YZbg),(Xab,Ca,YZbh),(Xab,Ca,YZbi),(Xab, Ca,YZbj),(Xab,Ca,YZbk),(Xab,Ca,YZbl),(Xab,Ca,YZbm),(Xab,Ca,YZbn),(Xab,Ca,YZbo), (Xab,Ca,YZbp),(Xab,Ca,YZbq),(Xab,Ca,YZbr),(Xab,Ca,YZbs),(Xab,Ca,YZbt),(Xab, Ca,YZbu),(Xab,Ca,YZbv),(Xab,Ca,YZbw),(Xab,Ca,YZbx),(Xab,Ca,YZby),(Xab, Ca,YZbz),(Xab,Ca,YZca),(Xab,Ca,YZcb),(Xab,Ca,YZcc),(Xab,Ca,YZcd),(Xab,Ca,YZce), (Xab,Ca,YZcf),(Xab,Ca,YZcg),(Xab,Ca,YZch),(Xab,Ca,YZci),(Xab,Ca,YZcj),(Xab, Ca,YZck),(Xab,Ca,YZcl),(Xab,Ca,YZcm),(Xab,Ca,YZcn),(Xab,Ca,YZco),(Xab,Ca, YZcp),(Xab,Ca,YZcq),(Xab,Ca,YZcr),(Xab,Ca,YZcs),(Xab,Ca,YZct),(Xab,Ca,YZcu), (Xab,Ca,YZcv),(Xab,Ca,YZcw),(Xab,Ca,YZcx),(Xab,Ca,YZcy),(Xab,Ca,YZcz),(Xab, Ca,YZda),(Xab,Ca,YZdb),(Xab,Ca,YZdc),(Xab,Ca,YZdd),(Xab,Ca,YZde),(Xab,Ca,YZdf), (Xab,Ca,YZdg),(Xab,Ca,YZdh),(Xab,Ca,YZdi),(Xab,Ca,YZdj),(Xab,Ca,YZdk),(Xab, Ca,YZdl),(Xab,Ca,YZdm),(Xab,Ca,YZdn),(Xab,Ca,YZdo),(Xab,Ca,YZdp),(Xab, Ca,YZdq),(Xab,Ca,YZdr),(Xab,Ca,YZds),(Xab,Ca,YZdt),(Xab,Ca,YZdu),(Xab,Ca,YZdv), (Xab,Ca,YZdw),(Xab,Ca,YZdx),(Xab,Ca,YZdy),(Xab,Ca,YZdz),(Xab,Ca,YZea),(Xab, Ca,YZeb),(Xab,Ca,YZec),(Xab,Ca,YZed),(Xab,Ca,YZee),(Xab,Ca,YZef),(Xab,Ca, YZeg),(Xab,Ca,YZeh),(Xab,Ca,YZei),(Xab,Ca,YZej),(Xab,Ca,YZek),(Xab,Ca,YZel), (Xab,Ca,YZem),(Xab,Ca,YZen),(Xab,Ca,YZeo),(Xab,Ca,YZep),(Xab,Ca,YZeq),(Xab, Ca,YZer),(Xab,Ca,YZes),(Xab,Ca,YZet),(Xab,Ca,YZeu),(Xab,Ca,YZev),(Xab,Ca,YZew), (Xab,Ca,YZex),(Xab,Ca,YZey),(Xab,Ca,YZez),(Xab,Ca,YZfa),(Xab,Ca,VZfb),(Xab, Ca,VZfc),(Xab,Ca,YZfd),(Xab,Ca,YZfe),(Xab,Ca,YZff),(Xab,Ca,YZfg),(Xab,Ca,YZfh), (Xab,Ca,YZfi),(Xab,Ca,YZfj),(Xab,Ca,YZfk),(Xab,Ca,YZfl),(Xab,Ca,YZfm),(Xab, Ca,YZfn),(Xab,Ca,YZfo),(Xab,Ca,YZfp),(Xab,Ca,YZfq),(Xab,Ca,YZfr),(Xab,Ca,YZfs), (Xab,Ca,YZft),(Xab,Ca,YZfu),(Xab,Ca,YZfv),(Xab,Ca,YZfw),(Xab,Ca,YZfx),(Xab, Ca,YZfy),(Xab,Ca,YZfz),(Xab,Ca,YZga),(Xab,Ca,YZgb),(Xab,Ca,YZgc),(Xab,Ca, YZgd),(Xab,Ca,YZge),(Xab,Ca,YZgf),(Xab,Ca,YZgg),(Xab,Ca,YZgh),(Xab,Ca,YZgi), (Xab,Ca,YZgj),(Xab,Ca,YZgk),(Xab,Ca,YZgl),(Xab,Ca,YZgm),(Xab,Ca,YZgn),(Xab, Ca,YZgo),(Xab,Ca,YZgp),(Xab,Ca,YZgq),(Xab,Ca,YZgr),(Xab,Ca,YZgs),(Xab,Ca,YZgt), (Xab,Cb,YZa),(Xab,Cb,YZb),(Xab,Cb,YZc),(Xab,Cb,YZd),(Xab,Cb,YZe),(Xab, Cb,YZf),(Xab,Cb,YZg),(Xab,Cb,YZh),(Xab,Cb,YZi),(Xab,Cb,YZj),(Xab,Cb,YZk),(Xab, Cb,YZl),(Xab,Cb,YZm),(Xab,Cb,YZn),(Xab,Cb,YZo),(Xab,Cb,YZp),(Xab,Cb,YZq), (Xab,Cb,YZr),(Xab,Cb,YZs),(Xab,Cb,YZt),(Xab,Cb,YZu),(Xab,Cb,YZv),(Xab,Cb,YZw), (Xab,Cb,YZx),(Xab,Cb,YZy),(Xab,Cb,YZz),(Xab,Cb,YZaa),(Xab,Cb,YZab),(Xab, Cb,YZac),(Xab,Cb,YZad),(Xab,Cb,YZae),(Xab,Cb,YZaf),(Xab,Cb,YZag),(Xab,Cb, YZah),(Xab,Cb,YZai),(Xab,Cb,YZaj),(Xab,Cb,YZak),(Xab,Cb,YZal),(Xab,Cb,YZam), (Xab,Cb,YZan),(Xab,Cb,YZao),(Xab,Cb,YZap),(Xab,Cb,YZaq),(Xab,Cb,YZar),(Xab, Cb,YZas),(Xab,Cb,YZat),(Xab,Cb,YZau),(Xab,Cb,YZav),(Xab,Cb,YZaw),(Xab,Cb,YZax), (Xab,Cb,YZay),(Xab,Cb,YZaz),(Xab,Cb,YZba),(Xab,Cb,YZbb),(Xab,Cb,YZbc), (Xab,Cb,YZbd),(Xab,Cb,YZbe),(Xab,Cb,YZbf),(Xab,Cb,YZbg),(Xab,Cb,YZbh),(Xab, Cb,YZbi),(Xab,Cb,YZbj),(Xab,Cb,YZbk),(Xab,Cb,YZbl),(Xab,Cb,YZbm),(Xab,Cb,YZbn), (Xab,Cb,YZbo),(Xab,Cb,YZbp),(Xab,Cb,YZbq),(Xab,Cb,YZbr),(Xab,Cb,YZbs), (Xab,Cb,YZbt),(Xab,Cb,YZbu),(Xab,Cb,YZbv),(Xab,Cb,YZbw),(Xab,Cb,YZbx),(Xab, Cb,YZby),(Xab,Cb,YZbz),(Xab,Cb,YZca),(Xab,Cb,YZcb),(Xab,Cb,YZcc),(Xab,Cb,YZcd), (Xab,Cb,YZce),(Xab,Cb,YZcf),(Xab,Cb,YZcg),(Xab,Cb,YZch),(Xab,Cb,YZci),(Xab, Cb,YZcj),(Xab,Cb,YZck),(Xab,Cb,YZcl),(Xab,Cb,YZcm),(Xab,Cb,YZcn),(Xab, Cb,YZco),(Xab,Cb,YZcp),(Xab,Cb,YZcq),(Xab,Cb,YZcr),(Xab,Cb,YZcs),(Xab,Cb,YZct), (Xab,Cb,YZcu),(Xab,Cb,YZcv),(Xab,Cb,YZcw),(Xab,Cb,YZcx),(Xab,Cb,YZcy), (Xab,Cb,YZcz),(Xab,Cb,YZda),(Xab,Cb,YZdb),(Xab,Cb,YZdc),(Xab,Cb,YZdd),(Xab, Cb,YZde),(Xab,Cb,YZdf),(Xab,Cb,YZdg),(Xab,Cb,YZdh),(Xab,Cb,YZdi),(Xab,Cb,YZdj), (Xab,Cb,YZdk),(Xab,Cb,YZdl),(Xab,Cb,YZdm),(Xab,Cb,YZdn),(Xab,Cb,YZdo), (Xab,Cb,YZdp),(Xab,Cb,YZdq),(Xab,Cb,YZdr),(Xab,Cb,YZds),(Xab,Cb,YZdt),(Xab, Cb,YZdu),(Xab,Cb,YZdv),(Xab,Cb,YZdw),(Xab,Cb,YZdx),(Xab,Cb,YZdy),(Xab,Cb, YZdz),(Xab,Cb,YZea),(Xab,Cb,YZeb),(Xab,Cb,YZec),(Xab,Cb,YZed),(Xab,Cb,YZee), (Xab,Cb,YZef),(Xab,Cb,YZeg),(Xab,Cb,YZeh),(Xab,Cb,YZei),(Xab,Cb,YZej),(Xab, Cb,YZek),(Xab,Cb,YZel),(Xab,Cb,YZem),(Xab,Cb,YZen),(Xab,Cb,YZeo),(Xab,Cb,YZep), (Xab,Cb,YZeq),(Xab,Cb,YZer),(Xab,Cb,YZes),(Xab,Cb,YZet),(Xab,Cb,YZeu),(Xab, Cb,YZev),(Xab,Cb,YZew),(Xab,Cb,YZex),(Xab,Cb,YZey),(Xab,Cb,YZez),(Xab, Cb,YZfa),(Xab,Cb,YZfb),(Xab,Cb,YZfc),(Xab,Cb,YZfd),(Xab,Cb,YZfe),(Xab,Cb,YZff), (Xab,Cb,YZfg),(Xab,Cb,YZfh),(Xab,Cb,YZfi),(Xab,Cb,YZfj),(Xab,Cb,YZfk),(Xab, Cb,YZfl),(Xab,Cb,YZfm),(Xab,Cb,YZfn),(Xab,Cb,YZfo),(Xab,Cb,YZfp),(Xab,Cb,YZfq), (Xab,Cb,YZfr),(Xab,Cb,YZfs),(Xab,Cb,YZft),(Xab,Cb,YZfu),(Xab,Cb,YZfv),(Xab, Cb,YZfw),(Xab,Cb,YZfx),(Xab,Cb,YZfy),(Xab,Cb,YZfz),(Xab,Cb,YZga),(Xab,Cb,YZgb), (Xab,Cb,YZgc),(Xab,Cb,YZgd),(Xab,Cb,YZge),(Xab,Cb,YZgf),(Xab,Cb,YZgg), (Xab,Cb,YZgh),(Xab,Cb,YZgi),(Xab,Cb,YZgj),(Xab,Cb,YZgk),(Xab,Cb,YZgl),(Xab, Cb,YZgm),(Xab,Cb,YZgn),(Xab,Cb,YZgo),(Xab,Cb,YZgp),(Xab,Cb,YZgq),(Xab,Cb, YZgr),(Xab,Cb,YZgs),(Xab,Cb,YZgt),(Xab,Cc,YZa),(Xab,Cc,YZb),(Xab,Cc,YZc),(Xab, Cc,YZd),(Xab,Cc,YZe),(Xab,Cc,YZf),(Xab,Cc,YZg),(Xab,Cc,YZh),(Xab,Cc,YZi),(Xab, Cc,YZj),(Xab,Cc,YZk),(Xab,Cc,YZl),(Xab,Cc,YZm),(Xab,Cc,YZn),(Xab,Cc,YZo), (Xab,Cc,YZp),(Xab,Cc,YZq),(Xab,Cc,YZr),(Xab,Cc,YZs),(Xab,Cc,YZt),(Xab,Cc, YZu),(Xab,Cc,YZv),(Xab,Cc,YZw),(Xab,Cc,YZx),(Xab,Cc,YZy),(Xab,Cc,YZz),(Xab, Cc,YZaa),(Xab,Cc,YZab),(Xab,Cc,YZac),(Xab,Cc,YZad),(Xab,Cc,YZae),(Xab,Cc,YZaf), (Xab,Cc,YZag),(Xab,Cc,YZah),(Xab,Cc,YZai),(Xab,Cc,YZaj),(Xab,Cc,YZak),(Xab, Cc,YZal),(Xab,Cc,YZam),(Xab,Cc,YZan),(Xab,Cc,YZao),(Xab,Cc,YZap),(Xab,Cc, YZaq),(Xab,Cc,YZar),(Xab,Cc,YZas),(Xab,Cc,YZat),(Xab,Cc,YZau),(Xab,Cc,YZav), (Xab,Cc,YZaw),(Xab,Cc,YZax),(Xab,Cc,YZay),(Xab,CG,YZaz),(Xab,Cc,YZba),(Xab, Cc,YZbb),(Xab,Cc,YZbc),(Xab,Cc,YZbd),(Xab,Cc,YZbe),(Xab,Cc,YZbf),(Xab,Cc,YZbg), (Xab,Cc,YZbh),(Xab,Cc,YZbi),(Xab,Cc,YZbj),(Xab,Cc,YZbk),(Xab,Cc,YZbl),(Xab, Cc,YZbm),(Xab,Cc,YZbn),(Xab,Cc,YZbo),(Xab,Cc,YZbp),(Xab,Cc,YZbq),(Xab, Cc,YZbr),(Xab,Cc,YZbs),(Xab,Cc,YZbt),(Xab,Cc,YZbu),(Xab,Cc,YZbv),(Xab,Cc,YZbw), (Xab,Cc,YZbx),(Xab,Cc,YZby),(Xab,Cc,YZbz),(Xab,Cc,YZca),(Xab,Cc,YZcb),(Xab, Cc,YZcc),(Xab,Cc,YZcd),(Xab,Cc,YZce),(Xab,Cc,YZcf),(Xab,Cc,YZcg),(Xab,Cc, YZch),(Xab,Cc,YZci),(Xab,Cc,YZcj),(Xab,Cc,YZck),(Xab,Cc,YZcl),(Xab,Cc,YZcm), (Xab,Cc,YZcn),(Xab,Cc,YZco),(Xab,Cc,YZcp),(Xab,Cc,YZcq),(Xab,Cc,YZcr),(Xab, Cc,YZcs),(Xab,Cc,YZct),(Xab,Cc,YZcu),(Xab,Cc,YZcv),(Xab,Cc,YZcw),(Xab,Cc,YZcx), (Xab,Cc,YZcy),(Xab,Cc,YZcz),(Xab,Cc,YZda),(Xab,Cc,YZdb),(Xab,Cc,YZdc),(Xab, Cc,YZdd),(Xab,Cc,YZde),(Xab,Cc,YZdf),(Xab,Cc,YZdg),(Xab,Cc,YZdh),(Xab,Cc, YZdi),(Xab,Cc,YZdj),(Xab,Cc,YZdk),(Xab,Cc,YZdl),(Xab,Cc,YZdm),(Xab,Cc,YZdn), (Xab,Cc,YZdo),(Xab,Cc,YZdp),(Xab,Cc,YZdq),(Xab,Cc,YZdr),(Xab,Cc,YZds),(Xab, Cc,YZdt),(Xab,Cc,YZdu),(Xab,Cc,YZdv),(Xab,Cc,YZdw),(Xab,Cc,YZdx),(Xab,Cc, YZdy),(Xab,Cc,YZdz),(Xab,Cc,YZea),(Xab,Cc,YZeb),(Xab,Cc,YZec),(Xab,Cc,YZed), (Xab,Cc,YZee),(Xab,Cc,YZef),(Xab,Cc,YZeg),(Xab,Cc,YZeh),(Xab,Cc,YZei),(Xab, Cc,YZej),(Xab,Cc,YZek),(Xab,Cc,YZel),(Xab,Cc,YZem),(Xab,Cc,YZen),(Xab,Cc,YZeo), (Xab,Cc,YZep),(Xab,Cc,YZeq),(Xab,Cc,YZer),(Xab,Cc,YZes),(Xab,Cc,YZet),(Xab, Cc,YZeu),(Xab,Cc,YZev),(Xab,Cc,YZew),(Xab,Cc,YZex),(Xab,Cc,YZey),(Xab,Cc, YZez),(Xab,Cc,YZfa),(Xab,Cc,YZfb),(Xab,Cc,YZfc),(Xab,Cc,YZfd),(Xab,Cc,YZfe),(Xab, Cc,YZff),(Xab,Cc,YZfg),(Xab,Cc,YZfh),(Xab,Cc,YZfi),(Xab,Cc,YZfj),(Xab,Cc, YZfk),(Xab,Cc,YZfl),(Xab,Cc,YZfm),(Xab,Cc,YZfn),(Xab,Cc,YZfo),(Xab,Cc,YZfp),(Xab, Cc,YZfq),(Xab,Cc,YZfr),(Xab,Cc,YZfs),(Xab,Cc,YZft),(Xab,Cc,YZfu),(Xab,Cc, YZfv),(Xab,Cc,YZfw),(Xab,Cc,YZfx),(Xab,Cc,YZfy),(Xab,Cc,YZfz),(Xab,Cc,YZga), (Xab,Cc,YZgb),(Xab,Cc,YZgc),(Xab,Cc,YZgd),(Xab,Cc,YZge),(Xab,Cc,YZgf),(Xab, Cc,YZgg),(Xab,Cc,YZgh),(Xab,Cc,YZgi),(Xab,Cc,YZgj),(Xab,Cc,YZgk),(Xab,Cc,YZgl), (Xab,Cc,YZgm),(Xab,Cc,YZgn),(Xab,Cc,YZgo),(Xab,Cc,YZgp),(Xab,Cc,YZgq),(Xab, Cc,YZgr),(Xab,Cc,YZgs),(Xab,Cc,YZgt),(Xab,Cd,YZa),(Xab,Cd,YZb),(Xab,Cd, YZc),(Xab,Cd,YZd),(Xab,Cd,YZe),(Xab,Cd,YZf),(Xab,Cd,YZg),(Xab,Cd,YZh),(Xab, Cd,YZi),(Xab,Cd,YZj),(Xab,Cd,YZk),(Xab,Cd,YZl),(Xab,Cd,YZm),(Xab,Cd,YZn),(Xab, Cd,YZo),(Xab,Cd,YZp),(Xab,Cd,YZq),(Xab,Cd,YZr),(Xab,Cd,YZs),(Xab,Cd,YZt), (Xab,Cd,YZu),(Xab,Cd,YZv),(Xab,Cd,YZw),(Xab,Cd,YZx),(Xab,Cd,YZy),(Xab,Cd, YZz),(Xab,Cd,YZaa),(Xab,Cd,YZab),(Xab,Cd,YZac),(Xab,Cd,YZad),(Xab,Cd,YZae), (Xab,Cd,YZaf),(Xab,Cd,YZag),(Xab,Cd,YZah),(Xab,Cd,YZai),(Xab,Cd,YZaj),(Xab,Cd, YZak),(Xab,Cd,YZal),(Xab,Cd,YZam),(Xab,Cd,YZan),(Xab,Cd,YZao),(Xab,Cd,YZap), (Xab,Cd,YZaq),(Xab,Cd,YZar),(Xab,Cd,YZas),(Xab,Cd,YZat),(Xab,Cd,YZau),(Xab, Cd,YZav),(Xab,Cd,YZaw),(Xab,Cd,YZax),(Xab,Cd,YZay),(Xab,Cd,YZaz),(Xab,Cd, YZba),(Xab,Cd,YZbb),(Xab,Cd,YZbc),(Xab,Cd,YZbd),(Xab,Cd,YZbe),(Xab,Cd,YZbf), (Xab,Cd,YZbg),(Xab,Cd,YZbh),(Xab,Cd,YZbi),(Xab,Cd,YZbj),(Xab,Cd,YZbk),(Xab, Cd,YZbl),(Xab,Cd,YZbm),(Xab,Cd,YZbn),(Xab,Cd,YZbo),(Xab,Cd,YZbp),(Xab,Cd, YZbq),(Xab,Cd,YZbr),(Xab,Cd,YZbs),(Xab,Cd,YZbt),(Xab,Cd,YZbu),(Xab,Cd,YZbv), (Xab,Cd,YZbw),(Xab,Cd,YZbx),(Xab,Cd,YZby),(Xab,Cd,YZbz),(Xab,Cd,YZca),(Xab, Cd,YZcb),(Xab,Cd,YZcc),(Xab,Cd,YZcd),(Xab,Cd,YZce),(Xab,Cd,YZcf),(Xab,Cd, YZcg),(Xab,Cd,YZch),(Xab,Cd,YZci),(Xab,Cd,YZcj),(Xab,Cd,YZck),(Xab,Cd,YZcl), (Xab,Cd,YZcm),(Xab,Cd,YZcn),(Xab,Cd,YZco),(Xab,Cd,YZcp),(Xab,Cd,YZcq),(Xab, Cd,YZcr),(Xab,Cd,YZcs),(Xab,Cd,YZct),(Xab,Cd,YZcu),(Xab,Cd,YZcv),(Xab,Cd, YZcw),(Xab,Cd,YZcx),(Xab,Cd,YZcy),(Xab,Cd,YZcz),(Xab,Cd,YZda),(Xab,Cd,YZdb), (Xab,Cd,YZdc),(Xab,Cd,YZdd),(Xab,Cd,YZde),(Xab,Cd,YZdf),(Xab,Cd,YZdg),(Xab, Cd,YZdh),(Xab,Cd,YZdi),(Xab,Cd,YZdj),(Xab,Cd,YZdk),(Xab,Cd,YZdl),(Xab,Cd, YZdm),(Xab,Cd,YZdn),(Xab,Cd,YZdo),(Xab,Cd,YZdp),(Xab,Cd,YZdq),(Xab,Cd,YZdr), (Xab,Cd,YZds),(Xab,Cd,YZdt),(Xab,Cd,YZdu),(Xab,Cd,YZdv),(Xab,Cd,YZdw),(Xab, Cd,YZdx),(Xab,Cd,YZdy),(Xab,Cd,YZdz),(Xab,Cd,YZea),(Xab,Cd,YZeb),(Xab, Cd,YZec),(Xab,Cd,YZed),(Xab,Cd,YZee),(Xab,Cd,YZef),(Xab,Cd,YZeg),(Xab,Cd,YZeh), (Xab,Cd,YZei),(Xab,Cd,YZej),(Xab,Cd,YZek),(Xab,Cd,YZel),(Xab,Cd,YZem),(Xab, Cd,YZen),(Xab,Cd,YZeo),(Xab,Cd,YZep),(Xab,Cd,YZeq),(Xab,Cd,YZer),(Xab, Cd,YZes),(Xab,Cd,YZet),(Xab,Cd,YZeu),(Xab,Cd,YZev),(Xab,Cd,YZew),(Xab,Cd,YZex), (Xab,Cd,YZey),(Xab,Cd,YZez),(Xab,Cd,YZfa),(Xab,Cd,YZfb),(Xab,Cd,YZfc),(Xab, Cd,YZfd),(Xab,Cd,YZfe),(Xab,Cd,YZff),(Xab,Cd,YZfg),(Xab,Cd,YZfh),(Xab,Cd, YZfi),(Xab,Cd,YZfj),(Xab,Cd,YZfk),(Xab,Cd,YZfl),(Xab,Cd,YZfm),(Xab,Cd,YZfn), (Xab,Cd,YZfo),(Xab,Cd,YZfp),(Xab,Cd,YZfq),(Xab,Cd,YZfr),(Xab,Cd,YZfs),(Xab,Cd, YZft),(Xab,Cd,YZfu),(Xab,Cd,YZfv),(Xab,Cd,YZfw),(Xab,Cd,YZfx),(Xab,Cd,YZfy), (Xab,Ca,YZfz),(Xab,Cd,YZga),(Xab,Cd,YZgb),(Xab,Cd,YZgc),(Xab,Cd,YZgd),(Xab, Cd,YZge),(Xab,Cd,YZgf),(Xab,Cd,YZgg),(Xab,Cd,YZgh),(Xab,Cd,YZgi),(Xab,Cd, YZgj),(Xab,Cd,YZgk),(Xab,Cd,YZgl),(Xab,Cd,YZgm),(Xab,Cd,YZgn),(Xab,Cd,YZgo), (Xab,Cd,YZgp),(Xab,Cd,YZgq),(Xab,Cd,YZgr),(Xab,Cd,YZgs),(Xab,Cd,YZgt),(Xab, Ce,YZa),(Xab,Ce,YZb),(Xab,Ce,YZc),(Xab,Ce,YZd),(Xab,Ce,YZe),(Xab,Ce,YZf),(Xab, Ce,YZg),(Xab,Ce,YZh),(Xab,Ce,YZi),(Xab,Ce,YZj),(Xab,Ce,YZk),(Xab,Ce,YZl), (Xab,Ce,YZm),(Xab,Ce,YZn),(Xab,Ce,YZo),(Xab,Ce,YZp),(Xab,Ce,YZq),(Xab,Ce,YZr), (Xab,Ce,YZs),(Xab,Ce,YZt),(Xab,Ce,YZu),(Xab,Ce,YZv),(Xab,Ce,YZw),(Xab,Ce, YZx),(Xab,Ce,YZy),(Xab,Ce,YZz),(Xab,Ce,YZaa),(Xab,Ce,YZab),(Xab,Ce,YZac),(Xab, Ce,YZad),(Xab,Ce,YZae),(Xab,Ce,YZaf),(Xab,Ce,YZag),(Xab,Ce,YZah),(Xab,Ce, YZai),(Xab,Ce,YZaj),(Xab,Ce,YZak),(Xab,Ce,YZal),(Xab,Ce,YZam),(Xab,Ce,YZan), (Xab,Ce,YZao),(Xab,Ce,YZap),(Xab,Ce,YZaq),(Xab,Ce,YZar),(Xab,Ce,YZas),(Xab, Ce,YZat),(Xab,Ce,YZau),(Xab,Ce,YZav),(Xab,Ce,YZaw),(Xab,Ce,YZax),(Xab,Ce,YZay), (Xab,Ce,YZaz),(Xab,Ce,YZba),(Xab,Ce,YZbb),(Xab,Ce,YZbc),(Xab,Ce,YZbd),(Xab, Ce,YZbe),(Xab,Ce,YZbf),(Xab,Ce,YZbg),(Xab,Ce,YZbh),(Xab,Ce,YZbi),(Xab,Ce, YZbj),(Xab,Ce,YZbk),(Xab,Ce,YZbl),(Xab,Ce,YZbm),(Xab,Ce,YZbn),(Xab,Ce,YZbo), (Xab,Ce,YZbp),(Xab,Ce,YZbq),(Xab,Ce,YZbr),(Xab,Ce,YZbs),(Xab,Ce,YZbt),(Xab, Ce,YZbu),(Xab,Ce,YZbv),(Xab,Ce,YZbw),(Xab,Ce,YZbx),(Xab,Ce,YZby),(Xab,Ce, YZbz),(Xab,Ce,YZca),(Xab,Ce,YZcb),(Xab,Ce,YZcc),(Xab,Ce,YZcd),(Xab,Ce,YZce), (Xab,Ce,YZcf),(Xab,Ce,YZcg),(Xab,Ce,YZch),(Xab,Ce,YZci),(Xab,Ce,YZcj),(Xab,Ce, YZck),(Xab,Ce,YZcl),(Xab,Ce,YZcm),(Xab,Ce,YZcn),(Xab,Ce,YZco),(Xab,Ce,YZcp), (Xab,Ce,YZcq),(Xab,Ce,YZcr),(Xab,Ce,YZcs),(Xab,Ce,YZct),(Xab,Ce,YZcu),(Xab, Ce,YZcv),(Xab,Ce,YZcw),(Xab,Ce,YZcx),(Xab,Ce,YZcy),(Xab,Ce,YZcz),(Xab,Ce,YZda), (Xab,Ce,YZdb),(Xab,Ce,YZdc),(Xab,Ce,YZdd),(Xab,Ce,YZde),(Xab,Ce,YZdf),(Xab, Ce,YZdg),(Xab,Ce,YZdh),(Xab,Ce,YZdi),(Xab,Ce,YZdj),(Xab,Ce,YZdk),(Xab,Ce, YZdl),(Xab,Ce,YZdm),(Xab,Ce,YZdn),(Xab,Ce,YZdo),(Xab,Ce,YZdp),(Xab,Ce,YZdq), (Xab,Ce,YZdr),(Xab,Ce,YZds),(Xab,Ce,YZdt),(Xab,CC,YZdu),(Xab,Ce,YZdv),(Xab, Ce,YZdw),(Xab,Ce,YZdx),(Xab,Ce,YZdy),(Xab,Ce,YZdz),(Xab,Ce,YZea),(Xab,Ce, YZeb),(Xab,Ce,YZec),(Xab,Ce,YZed),(Xab,Ce,YZee),(Xab,Ce,YZef),(Xab,Ce,YZeg), (Xab,Ce,YZeh),(Xab,Ce,YZei),(Xab,Ce,YZej),(Xab,Ce,YZek),(Xab,Ce,YZel),(Xab,Ce, YZem),(Xab,Ce,YZen),(Xab,Ce,YZeo),(Xab,Ce,YZep),(Xab,Ce,YZeq),(Xab,Ce,YZer), (Xab,Ce,YZes),(Xab,Ce,YZet),(Xab,Ce,YZeu),(Xab,Ce,YZev),(Xab,Ce,YZew),(Xab, Ce,YZex),(Xab,Ce,YZey),(Xab,Ce,YZez),(Xab,Ce,YZfa),(Xab,Ce,YZfb),(Xab,Ce, YZfc),(Xab,Ce,YZfd),(Xab,Ce,YZfe),(Xab,Ce,YZff),(Xab,Ce,YZfg),(Xab,Ce,YZfh),(Xab, Ce,YZfi),(Xab,Ce,YZfj),(Xab,Ce,YZfk),(Xab,Ce,YZfl),(Xab,Ce,YZfm),(Xab,Ce, YZfn),(Xab,Ce,YZfo),(Xab,Ce,YZfp),(Xab,Ce,YZfq),(Xab,Ce,YZfr),(Xab,Ce,YZfs),(Xab, Ce,YZft),(Xab,Ce,YZfu),(Xab,Ce,YZfv),(Xab,Ce,YZfw),(Xab,Ce,YZfx),(Xab,Ce, YZfy),(Xab,Ce,YZfz),(Xab,Ce,YZga),(Xab,Ce,YZgb),(Xab,Ce,YZgc),(Xab,Ce,YZgd), (Xab,Ce,YZge),(Xab,Ce,YZgf),(Xab,Ce,YZgg),(Xab,Ce,YZgh),(Xab,Ce,YZgi),(Xab, Ce,YZgj),(Xab,Ce,YZgk),(Xab,Ce,YZgl),(Xab,Ce,YZgm),(Xab,Ce,YZgn),(Xab,Ce,YZgo), (Xab,Ce,YZgp),(Xab,Ce,YZgq),(Xab,Ce,YZgr),(Xab,Ce,YZgs),(Xab,Ce,YZgt),(Xab, Cf,YZa),(Xab,Cf,YZb),(Xab,Cf,YZc),(Xab,Cf,YZd),(Xab,Cf,YZe),(Xab,Cf,YZf), (Xab,Cf,YZg),(Xab,Cf,YZh),(Xab,Cf,YZi),(Xab,Cf,YZj),(Xab,Cf,YZk),(Xab,Cf,YZl), (Xab,Cf,YZm),(Xab,Cf,YZn),(Xab,Cf,YZo),(Xab,Cf,YZp),(Xab,Cf,YZq),(Xab,Cf,YZr), (Xab,Cf,YZs),(Xab,Cf,YZt),(Xab,Cf,YZu),(Xab,Cf,YZv),(Xab,Cf,YZw),(Xab,Cf,YZx), (Xab,Cf,YZy),(Xab,Cf,YZz),(Xab,Cf,YZaa),(Xab,Cf,YZab),(Xab,Cf,YZac),(Xab, Cf,YZad),(Xab,Cf,YZae),(Xab,Cf,YZaf),(Xab,Cf,YZag),(Xab,Cf,YZah),(Xab,Cf,YZai), (Xab,Cf,YZaj),(Xab,Cf,YZak),(Xab,Cf,YZal),(Xab,Cf,YZam),(Xab,Cf,YZan),(Xab, Cf,YZao),(Xab,Cf,YZap),(Xab,Cf,YZaq),(Xab,Cf,YZar),(Xab,Cf,YZas),(Xab,Cf,YZat), (Xab,Cf,YZau),(Xab,Cf,YZav),(Xab,Cf,YZaw),(Xab,Cf,YZax),(Xab,Cf,YZay),(Xab, Cf,YZaz),(Xab,Cf,YZba),(Xab,Cf,YZbb),(Xab,Cf,YZbc),(Xab,Cf,YZbd),(Xab,Cf,YZbe), (Xab,Cf,YZbf),(Xab,Cf,YZbg),(Xab,Cf,YZbh),(Xab,Cf,YZbi),(Xab,Cf,YZbj),(Xab, Cf,YZbk),(Xab,Cf,YZbl),(Xab,Cf,YZbm),(Xab,Cf,YZbn),(Xab,Cf,YZbo),(Xab,Cf,YZbp), (Xab,Cf,YZbq),(Xab,Cf,YZbr),(Xab,Cf,YZbs),(Xab,Cf,YZbt),(Xab,Cf,YZbu),(Xab, Cf,YZbv),(Xab,Cf,YZbw),(Xab,Cf,YZbx),(Xab,Cf,YZby),(Xab,Cf,YZbz),(Xab,Cf,YZca), (Xab,Cf,YZcb),(Xab,Cf,YZcc),(Xab,Cf,YZcd),(Xab,Cf,YZce),(Xab,Cf,YZcf),(Xab, Cf,YZcg),(Xab,Cf,YZch),(Xab,Cf,YZci),(Xab,Cf,YZcj),(Xab,Cf,YZck),(Xab,Cf,YZcl), (Xab,Cf,YZcm),(Xab,Cf,YZcn),(Xab,Cf,YZco),(Xab,Cf,YZcp),(Xab,Cf,YZcq),(Xab, Cf,YZcr),(Xab,Cf,YZcs),(Xab,Cf,YZct),(Xab,Cf,YZcu),(Xab,Cf,YZcv),(Xab,Cf, YZcw),(Xab,Cf,YZcx),(Xab,Cf,YZcy),(Xab,Cf,YZcz),(Xab,Cf,YZda),(Xab,Cf,YZdb), (Xab,Cf,YZdc),(Xab,Cf,YZdd),(Xab,Cf,YZde),(Xab,Cf,YZdf),(Xab,Cf,YZdg),(Xab,Cf, YZdh),(Xab,Cf,YZdi),(Xab,Cf,YZdj),(Xab,Cf,YZdk),(Xab,Cf,YZdl),(Xab,Cf,YZdm), (Xab,Cf,YZdn),(Xab,Cf,YZdo),(Xab,Cf,YZdp),(Xab,Cf,YZdq),(Xab,Cf,YZdr),(Xab, Cf,YZds),(Xab,Cf,YZdt),(Xab,Cf,YZdu),(Xab,Cf,YZdv),(Xab,Cf,YZdw),(Xab,Cf,YZdx), (Xab,Cf,YZdy),(Xab,Cf,YZdz),(Xab,Cf,YZea),(Xab,Cf,YZeb),(Xab,Cf,YZec),(Xab, Cf,YZed),(Xab,Cf,YZee),(Xab,Cf,YZef),(Xab,Cf,YZeg),(Xab,Cf,YZeh),(Xab,Cf,YZei), (Xab,Cf,YZej),(Xab,Cf,YZek),(Xab,Cf,YZel),(Xab,Cf,YZem),(Xab,Cf,YZen),(Xab, Cf,YZeo),(Xab,Cf,YZep),(Xab,Cf,YZeq),(Xab,Cf,YZer),(Xab,Cf,YZes),(Xab,Cf,YZet), (Xab,Cf,YZeu),(Xab,Cf,YZev),(Xab,Cf,YZew),(Xab,Cf,YZex),(Xab,Cf,YZey),(Xab, Cf,YZez),(Xab,Cf,YZfa),(Xab,Cf,YZfb),(Xab,Cf,YZfc),(Xab,Cf,YZfd),(Xab,Cf,YZfe), (Xab,Cf,YZff),(Xab,Cf,YZfg),(Xab,Cf,YZfh),(Xab,Cf,YZfi),(Xab,Cf,YZfj),(Xab,Cf, YZfk),(Xab,Cf,YZfl),(Xab,Cf,YZfm),(Xab,Cf,YZfn),(Xab,Cf,YZfo),(Xab,Cf,YZfp),(Xab, Cf,YZfq),(Xab,Cf,YZfr),(Xab,Cf,YZfs),(Xab,Cf,YZft),(Xab,Cf,YZfu),(Xab,Cf,YZfv), (Xab,Cf,YZfw),(Xab,Cf,YZfx),(Xab,Cf,YZfy),(Xab,Cf,YZfz),(Xab,Cf,YZga),(Xab, Cf,YZgb),(Xab,Cf,YZgc),(Xab,Cf,YZgd),(Xab,Cf,YZge),(Xab,Cf,YZgf),(Xab,Cf,YZgg), (Xab,Cf,YZgh),(Xab,Cf,YZgi),(Xab,Cf,YZgj),(Xab,Cf,YZgk),(Xab,Cf,YZgl),(Xab, Cf,YZgm),(Xab,Cf,YZgn),(Xab,Cf,YZgo),(Xab,Cf,YZgp),(Xab,Cf,YZgq),(Xab,Cf, YZgr),(Xab,Cf,YZgs),(Xab,Cf,YZgt), (Xac,Ca,YZa),(Xac,Ca,YZb),(Xac,Ca,YZc),(Xac,Ca,YZd),(Xac,Ca,YZe),(Xac,Ca,YZf), (Xac,Ca,YZg),(Xac,Ca,YZh),(Xac,Ca,YZi),(Xac,Ca,YZj),(Xac,Ca,YZk),(Xac,Ca,YZl), (Xac,Ca,YZm),(Xac,Ca,YZn),(Xac,Ca,YZo),(Xac,Ca,YZp),(Xac,Ca,YZq),(Xac,Ca, YZr),(Xac,Ca,YZs),(Xac,Ca,YZt),(Xac,Ca,YZu),(Xac,Ca,YZv),(Xac,Ca,YZw),(Xac,Ca, YZx),(Xac,Ca,YZy),(Xac,Ca,YZz),(Xac,Ca,YZaa),(Xac,Ca,YZab),(Xac,Ca,YZac),(Xac, Ca,YZad),(Xac,Ca,YZae),(Xac,Ca,YZaf),(Xac,Ca,YZag),(Xac,Ca,YZah),(Xac,Ca, YZai),(Xac,Ca,YZaj),(Xac,Ca,YZak),(Xac,Ca,YZal),(Xac,Ca,YZam),(Xac,Ca,YZan), (Xac,Ca,YZao),(Xac,Ca,YZap),(Xac,Ca,YZaq),(Xac,Ca,YZar),(Xac,Ca,YZas),(Xac,Ca, YZat),(Xac,Ca,YZau),(Xac,Ca,YZav),(Xac,Ca,YZaw),(Xac,Ca,YZax),(Xac,Ca,YZay), (Xac,Ca,YZaz),(Xac,Ca,YZba),(Xac,Ca,YZbb),(Xac,Ca,YZbc),(Xac,Ca,YZbd),(Xac, Ca,YZbe),(Xac,Ca,YZbf),(Xac,Ca,YZbg),(Xac,Ca,YZbh),(Xac,Ca,YZbi),(Xac,Ca,YZbj), (Xac,Ca,YZbk),(Xac,Ca,YZbl),(Xac,Ca,YZbm),(Xac,Ca,YZbn),(Xac,Ca,YZbo),(Xac, Ca,YZbp),(Xac,Ca,YZbq),(Xac,Ca,YZbr),(Xac,Ca,YZbs),(Xac,Ca,YZbt),(Xac,Ca, YZbu),(Xac,Ca,YZbv),(Xac,Ca,YZbw),(Xac,Ca,YZbx),(Xac,Ca,YZby),(Xac,Ca,YZbz), (Xac,Ca,YZca),(Xac,Ca,YZcb),(Xac,Ca,YZcc),(Xac,Ca,YZcd),(Xac,Ca,YZce),(Xac, Ca,YZcf),(Xac,Ca,YZcg),(Xac,Ca,YZch),(Xac,Ca,YZci),(Xac,Ca,YZcj),(Xac,Ca,YZck), (Xac,Ca,YZcl),(Xac,Ca,YZcm),(Xac,Ca,YZcn),(Xac,Ca,YZco),(Xac,Ca,YZcp),(Xac, Ca,YZcq),(Xac,Ca,YZcr),(Xac,Ca,YZcs),(Xac,Ca,YZct),(Xac,Ca,YZcu),(Xac,Ca,YZcv), (Xac,Ca,YZcw),(Xac,Ca,YZcx),(Xac,Ca,YZcy),(Xac,Ca,YZcz),(Xac,Ca,YZda),(Xac, Ca,YZdb),(Xac,Ca,YZdc),(Xac,Ca,YZdd),(Xac,Ca,YZde),(Xac,Ca,YZdf),(Xac,Ca, YZdg),(Xac,Ca,YZdh),(Xac,Ca,YZdi),(Xac,Ca,YZdj),(Xac,Ca,YZdk),(Xac,Ca,YZdl),(Xac, Ca,YZdm),(Xac,Ca,YZdn),(Xac,Ca,YZdo),(Xac,Ca,YZdp),(Xac,Ca,YZdq),(Xac, Ca,YZdr),(Xac,Ca,YZds),(Xac,Ca,YZdt),(Xac,Ca,YZdu),(Xac,Ca,YZdv),(Xac,Ca,YZdw), (Xac,Ca,YZdx),(Xac,Ca,YZdy),(Xac,Ca,YZdz),(Xac,Ca,YZea),(Xac,Ca,YZeb),(Xac, Ca,YZec),(Xac,Ca,YZed),(Xac,Ca,YZee),(Xac,Ca,YZef),(Xac,Ca,YZeg),(Xac,Ca,YZeh), (Xac,Ca,YZei),(Xac,Ca,YZej),(Xac,Ca,YZek),(Xac,Ca,YZel),(Xac,Ca,YZem),(Xac, Ca,YZen),(Xac,Ca,YZeo),(Xac,Ca,YZep),(Xac,Ca,YZeq),(Xac,Ca,YZer),(Xac,Ca, YZes),(Xac,Ca,YZet),(Xac,Ca,YZeu),(Xac,Ca,YZev),(Xac,Ca,YZew),(Xac,Ca,YZex), (Xac,Ca,YZey),(Xac,Ca,YZez),(Xac,Ca,YZfa),(Xac,Ca,YZfb),(Xac,Ca,YZfc),(Xac,Ca, YZfd),(Xac,Ca,YZfe),(Xac,Ca,YZff),(Xac,Ca,YZfg),(Xac,Ca,YZfh),(Xac,Ca,YZfi),(Xac, Ca,YZfj),(Xac,Ca,YZfk),(Xac,Ca,YZfl),(Xac,Ca,YZfm),(Xac,Ca,YZfn),(Xac,Ca, YZfo),(Xac,Ca,YZfp),(Xac,Ca,YZfq),(Xac,Ca,YZfr),(Xac,Ca,YZfs),(Xac,Ca,YZft),(Xac, Ca,YZfu),(Xac,Ca,YZfv),(Xac,Ca,YZfw),(Xac,Ca,YZfx),(Xac,Ca,YZfy),(Xac,Ca,YZfz), (Xac,Ca,YZga),(Xac,Ca,YZgb),(Xac,Ca,YZgc),(Xac,Ca,YZgd),(Xac,Ca,YZge),(Xac, Ca,YZgf),(Xac,Ca,YZgg),(Xac,Ca,YZgh),(Xac,Ca,YZgi),(Xac,Ca,YZgj),(Xac,Ca, YZgk),(Xac,Ca,YZgl),(Xac,Ca,YZgm),(Xac,Ca,YZgn),(Xac,Ca,YZgo),(Xac,Ca,YZgp), (Xac,Ca,YZgq),(Xac,Ca,YZgr),(Xac,Ca,YZgs),(Xac,Ca,YZgt),(Xac,Cb,YZa),(Xac,Cb, YZb),(Xac,Cb,YZc),(Xac,Cb,YZd),(Xac,Cb,YZe),(Xac,Cb,YZf),(Xac,Cb,YZg),(Xac, Cb,YZh),(Xac,Cb,YZi),(Xac,Cb,YZj),(Xac,Cb,YZk),(Xac,Cb,YZl),(Xac,Cb,YZm),(Xac, Cb,YZn),(Xac,Cb,YZo),(Xac,Cb,YZp),(Xac,Cb,YZq),(Xac,Cb,YZr),(Xac,Cb,YZs), (Xac,Cb,YZt),(Xac,Cb,YZu),(Xac,Cb,YZv),(Xac,Cb,YZw),(Xac,Cb,YZx),(Xac,Cb,YZy), (Xac,Cb,YZz),(Xac,Cb,YZaa),(Xac,Cb,YZab),(Xac,Cb,YZac),(Xac,Cb,YZad),(Xac, Cb,YZae),(Xac,Cb,YZaf),(Xac,Cb,YZag),(Xac,Cb,YZah),(Xac,Cb,YZai),(Xac,Cb,YZaj), (Xac,Cb,YZak),(Xac,Cb,YZal),(Xac,Cb,YZam),(Xac,Cb,YZan),(Xac,Cb,YZao),(Xac, Cb,YZap),(Xac,Cb,YZaq),(Xac,Cb,YZar),(Xac,Cb,YZas),(Xac,Cb,YZat),(Xac,Cb, YZau),(Xac,Cb,YZav),(Xac,Cb,YZaw),(Xac,Cb,YZax),(Xac,Cb,YZay),(Xac,Cb,YZaz), (Xac,Cb,YZba),(Xac,Cb,YZbb),(Xac,Cb,YZbc),(Xac,Cb,YZbd),(Xac,Cb,YZbe),(Xac, Cb,YZbf),(Xac,Cb,YZbg),(Xac,Cb,YZbh),(Xac,Cb,YZbi),(Xac,Cb,YZbj),(Xac,Cb,YZbk), (Xac,Cb,YZbl),(Xac,Cb,YZbm),(Xac,Cb,YZbn),(Xac,Cb,YZbo),(Xac,Cb,YZbp), (Xac,Cb,YZbq),(Xac,Cb,YZbr),(Xac,Cb,YZbs),(Xac,Cb,YZbt),(Xac,Cb,YZbu),(Xac,Cb, YZbv),(Xac,Cb,YZbw),(Xac,Cb,YZbx),(Xac,Cb,YZby),(Xac,Cb,YZbz),(Xac,Cb,YZca), (Xac,Cb,YZcb),(Xac,Cb,YZcc),(Xac,Cb,YZcd),(Xac,Cb,YZce),(Xac,Cb,YZcf),(Xac, Cb,YZcg),(Xac,Cb,YZch),(Xac,Cb,YZci),(Xac,Cb,YZcj),(Xac,Cb,YZck),(Xac,Cb, YZcl),(Xac,Cb,YZcm),(Xac,Cb,YZcn),(Xac,Cb,YZco),(Xac,Cb,YZcp),(Xac,Cb,YZcq), (Xac,Cb,YZcr),(Xac,Cb,YZcs),(Xac,Cb,YZct),(Xac,Cb,YZcu),(Xac,Cb,YZcv),(Xac, Cb,YZcw),(Xac,Cb,YZcx),(Xac,Cb,YZcy),(Xac,Cb,YZcz),(Xac,Cb,YZda),(Xac,Cb,YZdb), (Xac,Cb,YZdc),(Xac,Cb,YZdd),(Xac,Cb,YZde),(Xac,Cb,YZdf),(Xac,Cb,YZdg),(Xac, Cb,YZdh),(Xac,Cb,YZdi),(Xac,Cb,YZdj),(Xac,Cb,YZdk),(Xac,Cb,YZdl),(Xac,Cb, YZdm),(Xac,Cb,YZdn),(Xac,Cb,YZdo),(Xac,Cb,YZdp),(Xac,Cb,YZdq),(Xac,Cb,YZdr), (Xac,Cb,YZds),(Xac,Cb,Yzdt),(Xac,Cb,YZdu),(Xac,Cb,YZdv),(Xac,Cb,YZdw),(Xac, Cb,YZdx),(Xac,Cb,YZdy),(Xac,Cb,YZdz),(Xac,Cb,YZea),(Xac,Cb,YZeb),(Xac,Cb, YZec),(Xac,Cb,YZed),(Xac,Cb,YZee),(Xac,Cb,YZef),(Xac,Cb,YZeg),(Xac,Cb,YZeh), (Xac,Cb,YZei),(Xac,Cb,YZej),(Xac,Cb,YZek),(Xac,Cb,YZel),(Xac,Cb,YZem),(Xac,Cb, YZen),(Xac,Cb,YZeo),(Xac,Cb,YZep),(Xac,Cb,YZeq),(Xac,Cb,YZer),(Xac,Cb,YZes), (Xac,Cb,YZet),(Xac,Cb,YZeu),(Xac,Cb,YZev),(Xac,Cb,YZew),(Xac,Cb,YZex),(Xac, Cb,YZey),(Xac,Cb,YZez),(Xac,Cb,YZfa),(Xac,Cb,YZfb),(Xac,Cb,YZfc),(Xac,Cb,YZfd), (Xac,Cb,YZfe),(Xac,Cb,YZff),(Xac,Cb,YZfg),(Xac,Cb,YZfh),(Xac,Cb,YZfi),(Xac, Cb,YZfj),(Xac,Cb,YZfk),(Xac,Cb,YZfl),(Xac,Cb,YZfm),(Xac,Cb,YZfn),(Xac,Cb,YZfo), (Xac,Cb,YZfp),(Xac,Cb,YZfq),(Xac,Cb,YZfr),(Xac,Cb,YZfs),(Xac,Cb,YZft),(Xac, Cb,YZfu),(Xac,Cb,YZfv),(Xac,Cb,YZfw),(Xac,Cb,YZfx),(Xac,Cb,YZfy),(Xac,Cb,YZfz), (Xac,Cb,YZga),(Xac,Cb,YZgb),(Xac,Cb,YZgc),(Xac,Cb,YZgd),(Xac,Cb,YZge),(Xac, Cb,YZgf),(Xac,Cb,YZgg),(Xac,Cb,YZgh),(Xac,Cb,YZgi),(Xac,Cb,YZgj),(Xac,Cb, YZgk),(Xac,Cb,YZgl),(Xac,Cb,YZgm),(Xac,Cb,YZgn),(Xac,Cb,YZgo),(Xac,Cb,YZgp), (Xac,Cb,YZgq),(Xac,Cb,YZgr),(Xac,Cb,YZgs),(Xac,Cb,YZgt),(Xac,Cc,YZa),(Xac, Cc,YZb),(Xac,Cc,YZc),(Xac,Cc,YZd),(Xac,Cc,YZe),(Xac,Cc,YZf),(Xac,Cc,YZg),(Xac, Cc,YZh),(Xac,Cc,YZi),(Xac,Cc,YZj),(Xac,Cc,YZk),(Xac,Cc,YZl),(Xac,Cc,YZm), (Xac,Cc,YZn),(Xac,Cc,YZo),(Xac,Cc,YZp),(Xac,Cc,YZq),(Xac,Cc,YZr),(Xac,Cc,YZs), (Xac,Cc,YZt),(Xac,Cc,YZu),(Xac,Cc,YZv),(Xac,Cc,YZw),(Xac,Cc,YZx),(Xac,Cc, YZy),(Xac,Cc,YZz),(Xac,Cc,YZaa),(Xac,Cc,YZab),(Xac,Cc,YZac),(Xac,Cc,YZad),(Xac, Cc,YZae),(Xac,Cc,YZaf),(Xac,Cc,YZag),(Xac,Cc,YZah),(Xac,Cc,YZai),(Xac,Cc,YZaj), (Xac,Cc,YZak),(Xac,Cc,YZal),(Xac,Cc,YZam),(Xac,Cc,YZan),(Xac,Cc,YZao),(Xac, Cc,YZap),(Xac,Cc,YZaq),(Xac,Cc,YZar),(Xac,Cc,YZas),(Xac,Cc,YZat),(Xac,Cc, YZau),(Xac,Cc,YZav),(Xac,Cc,YZaw),(Xac,Cc,YZax),(Xac,Cc,YZay),(Xac,Cc,YZaz), (Xac,Cc,YZba),(Xac,Cc,YZbb),(Xac,Cc,YZbc),(Xac,Cc,YZbd),(Xac,Cc,YZbe),(Xac, Cc,YZbf),(Xac,Cc,YZbg),(Xac,Cc,YZbh),(Xac,Cc,YZbi),(Xac,Cc,YZbj),(Xac,Cc,YZbk), (Xac,Cc,YZbl),(Xac,Cc,YZbm),(Xac,Cc,YZbn),(Xac,Cc,YZbo),(Xac,Cc,YZbp),(Xac, Cc,YZbq),(Xac,Cc,YZbr),(Xac,Cc,YZbs),(Xac,Cc,YZbt),(Xac,Cc,YZbu),(Xac,Cc,YZbv), (Xac,Cc,YZbw),(Xac,Cc,YZbx),(Xac,Cc,YZby),(Xac,Cc,YZbz),(Xac,Cc,YZca),(Xac, Cc,YZcb),(Xac,Cc,YZcc),(Xac,Cc,YZcd),(Xac,Cc,YZce),(Xac,Cc,YZcf),(Xac,Cc, YZcg),(Xac,Cc,YZch),(Xac,Cc,YZci),(Xac,Cc,YZcj),(Xac,Cc,YZck),(Xac,Cc,YZcl),(Xac, Cc,YZcm),(Xac,Cc,YZcn),(Xac,Cc,YZco),(Xac,Cc,YZcp),(Xac,Cc,YZcq),(Xac,Cc, YZcr),(Xac,Cc,YZcs),(Xac,Cc,YZct),(Xac,Cc,YZcu),(Xac,Cc,YZcv),(Xac,Cc,YZcw), (Xac,Cc,YZcx),(Xac,Cc,YZcy),(Xac,Cc,YZcz),(Xac,Cc,YZda),(Xac,Cc,YZdb),(Xac, Cc,YZdc),(Xac,Cc,YZdd),(Xac,Cc,YZde),(Xac,Cc,YZdf),(Xac,Cc,YZdg),(Xac,Cc,YZdh), (Xac,Cc,YZdi),(Xac,Cc,YZdj),(Xac,Cc,YZdk),(Xac,Cc,YZdl),(Xac,Cc,YZdm),(Xac, Cc,YZdn),(Xac,Cc,YZdo),(Xac,Cc,YZdp),(Xac,Cc,YZdq),(Xac,Cc,YZdr),(Xac,Cc, YZds),(Xac,Cc,Yzdt),(Xac,Cc,YZdu),(Xac,Cc,YZdv),(Xac,Cc,YZdw),(Xac,Cc,YZdx), (Xac,Cc,YZdy),(Xac,Cc,YZdz),(Xac,Cc,YZea),(Xac,Cc,YZeb),(Xac,Cc,YZec),(Xac,Cc, YZed),(Xac,Cc,YZee),(Xac,Cc,YZef),(Xac,Cc,YZeg),(Xac,Cc,YZeh),(Xac,Cc,YZei), (Xac,Cc,YZej),(Xac,Cc,YZek),(Xac,Cc,YZel),(Xac,Cc,YZem),(Xac,Cc,YZen),(Xac, Cc,YZeo),(Xac,Cc,YZep),(Xac,Cc,YZeq),(Xac,Cc,YZer),(Xac,Cc,YZes),(Xac,Cc,YZet), (Xac,Cc,YZeu),(Xac,Cc,YZev),(Xac,Cc,YZew),(Xac,Cc,YZex),(Xac,Cc,YZey),(Xac, Cc,YZez),(Xac,Cc,YZfa),(Xac,Cc,YZfb),(Xac,Cc,YZfc),(Xac,Cc,YZfd),(Xac,Cc,YZfe), (Xac,Cc,YZff),(Xac,Cc,YZfg),(Xac,Cc,YZfh),(Xac,Cc,YZfi),(Xac,Cc,YZfj),(Xac, Cc,YZfk),(Xac,Cc,YZfl),(Xac,Cc,YZfm),(Xac,Cc,YZfn),(Xac,Cc,YZfo),(Xac,Cc,YZfp), (Xac,Cc,YZfq),(Xac,Cc,YZfr),(Xac,Cc,YZfs),(Xac,Cc,YZft),(Xac,Cc,YZfu),(Xac,Cc, YZfv),(Xac,Cc,YZfw),(Xac,Cc,YZfx),(Xac,Cc,YZfy),(Xac,Cc,YZfz),(Xac,Cc,YZga), (Xac,Cc,YZgb),(Xac,Cc,YZgc),(Xac,Cc,YZgd),(Xac,Cc,YZge),(Xac,Cc,YZgf),(Xac, Cc,YZgg),(Xac,Cc,YZgh),(Xac,Cc,YZgi),(Xac,Cc,YZgj),(Xac,Cc,YZgk),(Xac,Cc,YZgl), (Xac,Cc,YZgm),(Xac,Cc,YZgn),(Xac,Cc,YZgo),(Xac,Cc,YZgp),(Xac,Cc,YZgq),(Xac, Cc,YZgr),(Xac,Cc,YZgs),(Xac,Cc,YZgt),(Xac,Cd,YZa),(Xac,Cd,YZb),(Xac,Cd,YZc), (Xac,Cd,YZd),(Xac,Cd,YZe),(Xac,Cd,YZf),(Xac,Cd,YZg),(Xac,Cd,YZh),(Xac,Cd, YZi),(Xac,Cd,YZj),(Xac,Cd,YZk),(Xac,Cd,YZl),(Xac,Cd,YZm),(Xac,Cd,YZn),(Xac, Cd,YZo),(Xac,Cd,YZp),(Xac,Cd,YZq),(Xac,Cd,YZr),(Xac,Cd,YZs),(Xac,Cd,YZt),(Xac, Cd,YZu),(Xac,Cd,YZv),(Xac,Cd,YZw),(Xac,Cd,YZx),(Xac,Cd,YZy),(Xac,Cd,YZz), (Xac,Cd,YZaa),(Xac,Cd,YZab),(Xac,Cd,YZac),(Xac,Cd,YZad),(Xac,Cd,YZae),(Xac, Cd,YZaf),(Xac,Cd,YZag),(Xac,Cd,YZah),(Xac,Cd,YZai),(Xac,Cd,YZaj),(Xac,Cd,YZak), (Xac,Cd,YZal),(Xac,Cd,YZam),(Xac,Cd,YZan),(Xac,Cd,YZao),(Xac,Cd,YZap),(Xac, Cd,YZaq),(Xac,Cd,YZar),(Xac,Cd,YZas),(Xac,Cd,YZat),(Xac,Cd,YZau),(Xac,Cd,YZav), (Xac,Cd,YZaw),(Xac,Cd,YZax),(Xac,Cd,YZay),(Xac,Cd,YZaz),(Xac,Cd,YZba),(Xac, Cd,YZbb),(Xac,Cd,YZbc),(Xac,Cd,YZbd),(Xac,Cd,YZbe),(Xac,Cd,YZbf),(Xac,Cd, YZbg),(Xac,Cd,YZbh),(Xac,Cd,YZbi),(Xac,Cd,YZbj),(Xac,Cd,YZbk),(Xac,Cd,YZbl), (Xac,Cd,YZbm),(Xac,Cd,YZbn),(Xac,Cd,YZbo),(Xac,Cd,YZbp),(Xac,Cd,YZbq),(Xac, Cd,YZbr),(Xac,Cd,YZbs),(Xac,Cd,YZbt),(Xac,Cd,YZbu),(Xac,Cd,YZbv),(Xac,Cd, YZbw),(Xac,Cd,YZbx),(Xac,Cd,YZby),(Xac,Cd,YZbz),(Xac,Cd,YZca),(Xac,Cd,YZcb), (Xac,Cd,YZcc),(Xac,Cd,YZcd),(Xac,Cd,YZce),(Xac,Cd,YZcf),(Xac,Cd,YZcg),(Xac, Cd,YZch),(Xac,Cd,YZci),(Xac,Cd,YZcj),(Xac,Cd,YZck),(Xac,Cd,YZcl),(Xac,Cd,YZcm), (Xac,Cd,YZcn),(Xac,Cd,YZco),(Xac,Cd,YZcp),(Xac,Cd,YZcq),(Xac,Cd,YZcr),(Xac, Cd,YZcs),(Xac,Cd,YZct),(Xac,Cd,YZcu),(Xac,Cd,YZcv),(Xac,Cd,YZcw),(Xac,Cd, YZcx),(Xac,Cd,YZcy),(Xac,Cd,YZcz),(Xac,Cd,YZda),(Xac,Cd,YZdb),(Xac,Cd,YZdc), (Xac,Cd,YZdd),(Xac,Cd,YZde),(Xac,Cd,YZdf),(Xac,Cd,YZdg),(Xac,Cd,YZdh),(Xac, Cd,YZdi),(Xac,Cd,YZdj),(Xac,Cd,YZdk),(Xac,Cd,YZdl),(Xac,Cd,YZdm),(Xac,Cd,YZdn), (Xac,Cd,YZdo),(Xac,Cd,YZdp),(Xac,Cd,YZdq),(Xac,Cd,YZdr),(Xac,Cd,YZds),(Xac, Cd,Yzdt),(Xac,Cd,YZdu),(Xac,Cd,YZdv),(Xac,Cd,YZdw),(Xac,Cd,YZdx),(Xac,Cd, YZdy),(Xac,Cd,YZdz),(Xac,Cd,YZea),(Xac,Cd,YZeb),(Xac,Cd,YZec),(Xac,Cd,YZed), (Xac,Cd,YZee),(Xac,Cd,YZef),(Xac,Cd,YZeg),(Xac,Cd,YZeh),(Xac,Cd,YZei),(Xac, Cd,YZej),(Xac,Cd,YZek),(Xac,Cd,YZel),(Xac,Cd,YZem),(Xac,Cd,YZen),(Xac,Cd,YZeo), (Xac,Cd,YZep),(Xac,Cd,YZeq),(Xac,Cd,YZer),(Xac,Cd,YZes),(Xac,Cd,YZet),(Xac, Cd,YZeu),(Xac,Cd,YZev),(Xac,Cd,YZew),(Xac,Cd,YZex),(Xac,Cd,YZey),(Xac,Cd, YZez),(Xac,Cd,YZfa),(Xac,Cd,YZfb),(Xac,Cd,YZfc),(Xac,Cd,YZfd),(Xac,Cd,YZfe),(Xac, Cd,YZff),(Xac,Cd,YZfg),(Xac,Cd,YZfh),(Xac,Cd,YZfi),(Xac,Cd,YZfj),(Xac,Cd, YZfk),(Xac,Cd,YZfl),(Xac,Cd,YZfm),(Xac,Cd,YZfn),(Xac,Cd,YZfo),(Xac,Cd,YZfp),(Xac, Cd,YZfq),(Xac,Cd,YZfr),(Xac,Cd,YZfs),(Xac,Cd,YZft),(Xac,Cd,YZfu),(Xac,Cd, YZfv),(Xac,Cd,YZfw),(Xac,Cd,YZfx),(Xac,Cd,YZfy),(Xac,Cd,YZfz),(Xac,Cd,YZga), (Xac,Cd,YZgb),(Xac,Cd,YZgc),(Xac,Cd,YZgd),(Xac,Cd,YZge),(Xac,Cd,YZgf),(Xac, Cd,YZgg),(Xac,Cd,YZgh),(Xac,Cd,YZgi),(Xac,Cd,YZgj),(Xac,Cd,YZgk),(Xac,Cd,YZgl), (Xac,Cd,YZgm),(Xac,Cd,YZgn),(Xac,Cd,YZgo),(Xac,Cd,YZgp),(Xac,Cd,YZgq),(Xac, Cd,YZgr),(Xac,Cd,YZgs),(Xac,Cd,YZgt),(Xac,Ce,YZa),(Xac,Ce,YZb),(Xac,Ce,YZc), (Xac,Ce,YZd),(Xac,Ce,YZe),(Xac,Ce,YZf),(Xac,Ce,YZg),(Xac,Ce,YZh),(Xac,Ce, YZi),(Xac,Ce,YZj),(Xac,Ce,YZk),(Xac,Ce,YZl),(Xac,Ce,YZm),(Xac,Ce,YZn),(Xac,Ce, YZo),(Xac,Ce,YZp),(Xac,Ce,YZq),(Xac,Ce,YZr),(Xac,Ce,YZs),(Xac,Ce,YZt),(Xac, Ce,YZu),(Xac,Ce,YZv),(Xac,Ce,YZw),(Xac,Ce,YZx),(Xac,Ce,YZy),(Xac,Ce,YZz),(Xac, Ce,YZaa),(Xac,Ce,YZab),(Xac,Ce,YZac),(Xac,Ce,YZad),(Xac,Ce,YZae),(Xac,Ce, YZaf),(Xac,Ce,YZag),(Xac,Ce,YZah),(Xac,Ce,YZai),(Xac,Ce,YZaj),(Xac,Ce,YZak),(Xac, Ce,YZal),(Xac,Ce,YZam),(Xac,Ce,YZan),(Xac,Ce,YZao),(Xac,Ce,YZap),(Xac,Ce, YZaq),(Xac,Ce,YZar),(Xac,Ce,YZas),(Xac,Ce,YZat),(Xac,Ce,YZau),(Xac,Ce,YZav), (Xac,Ce,YZaw),(Xac,Ce,YZax),(Xac,Ce,YZay),(Xac,Ce,YZaz),(Xac,Ce,YZba),(Xac, Ce,YZbb),(Xac,Ce,YZbc),(Xac,Ce,YZbd),(Xac,Ce,YZbe),(Xac,Ce,YZbf),(Xac,Ce,YZbg), (Xac,Ce,YZbh),(Xac,Ce,YZbi),(Xac,Ce,YZbj),(Xac,Ce,YZbk),(Xac,Ce,YZbl),(Xac, Ce,YZbm),(Xac,Ce,YZbn),(Xac,Ce,YZbo),(Xac,Ce,YZbp),(Xac,Ce,YZbq),(Xac,Ce, YZbr),(Xac,Ce,YZbs),(Xac,Ce,YZbt),(Xac,Ce,YZbu),(Xac,Ce,YZbv),(Xac,Ce,YZbw), (Xac,Ce,YZbx),(Xac,Ce,YZby),(Xac,Ce,YZbz),(Xac,Ce,YZca),(Xac,Ce,YZcb),(Xac,Ce, YZcc),(Xac,Ce,YZcd),(Xac,Ce,YZce),(Xac,Ce,YZcf),(Xac,Ce,YZcg),(Xac,Ce,YZch), (Xac,Ce,YZci),(Xac,Ce,YZcj),(Xac,Ce,YZck),(Xac,Ce,YZcl),(Xac,Ce,YZcm),(Xac, Ce,YZcn),(Xac,Ce,YZco),(Xac,Ce,YZcp),(Xac,Ce,YZcq),(Xac,Ce,YZer),(Xac,Ce,YZcs), (Xac,Ce,YZct),(Xac,Ce,YZcu),(Xac,Ce,YZcv),(Xac,Ce,YZcw),(Xac,Ce,YZcx),(Xac, Ce,YZcy),(Xac,Ce,YZcz),(Xac,Ce,YZda),(Xac,Ce,YZdb),(Xac,Ce,YZdc),(Xac,Ce,YZdd), (Xac,Ce,YZde),(Xac,Ce,YZdf),(Xac,Ce,YZdg),(Xac,Ce,YZdh),(Xac,Ce,YZdi),(Xac, Ce,YZdj),(Xac,Ce,YZdk),(Xac,Ce,YZdl),(Xac,Ce,YZdm),(Xac,Ce,YZdn),(Xac,Ce, YZdo),(Xac,Ce,YZdp),(Xac,Ce,YZdq),(Xac,Ce,YZdr),(Xac,Ce,YZds),(Xac,Ce,Yzdt), (Xac,Ce,YZdu),(Xac,Ce,YZdv),(Xac,Ce,YZdw),(Xac,Ce,YZdx),(Xac,Ce,YZdy),(Xac, Ce,YZdz),(Xac,Ce,YZea),(Xac,Ce,YZeb),(Xac,Ce,YZec),(Xac,Ce,YZed),(Xac,Ce,YZee), (Xac,Ce,YZef),(Xac,Ce,YZeg),(Xac,Ce,YZeh),(Xac,Ce,YZei),(Xac,Ce,YZej),(Xac, Ce,YZek),(Xac,Ce,YZel),(Xac,Ce,YZem),(Xac,Ce,YZen),(Xac,Ce,YZeo),(Xac,Ce, YZep),(Xac,Ce,YZeq),(Xac,Ce,YZer),(Xac,Ce,YZes),(Xac,Ce,YZet),(Xac,Ce,YZeu),(Xac, Ce,YZev),(Xac,Ce,YZew),(Xac,Ce,YZex),(Xac,Ce,YZey),(Xac,Ce,YZez),(Xac,Ce, YZfa),(Xac,Ce,YZfb),(Xac,Ce,YZfc),(Xac,Ce,YZfd),(Xac,Ce,YZfe),(Xac,Ce,YZff),(Xac, Ce,YZfg),(Xac,Ce,YZfh),(Xac,Ce,YZfi),(Xac,Ce,YZfj),(Xac,Ce,YZfk),(Xac,Ce, YZfl),(Xac,Ce,YZfm),(Xac,Ce,YZfn),(Xac,Ce,YZfo),(Xac,Ce,YZfp),(Xac,Ce,YZfq),(Xac, Ce,YZfr),(Xac,Ce,YZfs),(Xac,Ce,YZft),(Xac,Ce,YZfu),(Xac,Ce,YZfv),(Xac,Ce,YZfw), (Xac,Ce,YZfx),(Xac,Ce,YZfy),(Xac,Ce,YZfz),(Xac,Ce,YZga),(Xac,Ce,YZgb),(Xac, Ce,YZgc),(Xac,Ce,YZgd),(Xac,Ce,YZge),(Xac,Ce,YZgf),(Xac,Ce,YZgg),(Xac,Ce, YZgh),(Xac,Ce,YZgi),(Xac,Ce,YZgj),(Xac,Ce,YZgk),(Xac,Ce,YZgl),(Xac,Ce,YZgm), (Xac,Ce,YZgn),(Xac,Ce,YZgo),(Xac,Ce,YZgp),(Xac,Ce,YZgq),(Xac,Ce,YZgr),(Xac, Ce,YZgs),(Xac,Ce,YZgt),(Xac,Cf,YZa),(Xac,Cf,YZb),(Xac,Cf,YZc),(Xac,Cf,YZd),(Xac, Cf,YZe),(Xac,Cf,YZf),(Xac,Cf,YZg),(Xac,Cf,YZh),(Xac,Cf,YZi),(Xac,Cf,YZj),(Xac, Cf,YZk),(Xac,Cf,YZl),(Xac,Cf,YZm),(Xac,Cf,YZn),(Xac,Cf,YZo),(Xac,Cf,YZp),(Xac, Cf,YZq),(Xac,Cf,YZr),(Xac,Cf,YZs),(Xac,Cf,YZt),(Xac,Cf,YZu),(Xac,Cf,YZv),(Xac, Cf,YZw),(Xac,Cf,YZx),(Xac,Cf,YZy),(Xac,Cf,YZz),(Xac,Cf,YZaa),(Xac,Cf,YZab), (Xac,Cf,YZac),(Xac,Cf,YZad),(Xac,Cf,YZae),(Xac,Cf,YZaf),(Xac,Cf,YZag),(Xac,Cf, YZah),(Xac,Cf,YZai),(Xac,Cf,YZaj),(Xac,Cf,YZak),(Xac,Cf,YZal),(Xac,Cf,YZam),(Xac, Cf,YZan),(Xac,Cf,YZao),(Xac,Cf,YZap),(Xac,Cf,YZaq),(Xac,Cf,YZar),(Xac,Cf, YZas),(Xac,Cf,YZat),(Xac,Cf,YZau),(Xac,Cf,YZav),(Xac,Cf,YZaw),(Xac,Cf,YZax),(Xac, Cf,YZay),(Xac,Cf,YZaz),(Xac,Cf,YZba),(Xac,Cf,YZbb),(Xac,Cf,YZbc),(Xac,Cf, YZbd),(Xac,Cf,YZbe),(Xac,Cf,YZbf),(Xac,Cf,YZbg),(Xac,Cf,YZbh),(Xac,Cf,YZbi),(Xac, Cf,YZbj),(Xac,Cf,YZbk),(Xac,Cf,YZbl),(Xac,Cf,YZbm),(Xac,Cf,YZbn),(Xac,Cf, YZbo),(Xac,Cf,YZbp),(Xac,Cf,YZbq),(Xac,Cf,YZbr),(Xac,Cf,YZbs),(Xac,Cf,YZbt),(Xac, Cf,YZbu),(Xac,Cf,YZbv),(Xac,Cf,YZbw),(Xac,Cf,YZbx),(Xac,Cf,YZby),(Xac,Cf, YZbz),(Xac,Cf,YZca),(Xac,Cf,YZcb),(Xac,Cf,YZcc),(Xac,Cf,YZcd),(Xac,Cf,YZce),(Xac, Cf,YZcf),(Xac,Cf,YZcg),(Xac,Cf,YZch),(Xac,Cf,YZci),(Xac,Cf,YZcj),(Xac,Cf,YZck), (Xac,Cf,YZcl),(Xac,Cf,YZcm),(Xac,Cf,YZcn),(Xac,Cf,YZco),(Xac,Cf,YZcp),(Xac, Cf,YZcq),(Xac,Cf,YZcr),(Xac,Cf,YZcs),(Xac,Cf,YZct),(Xac,Cf,YZcu),(Xac,Cf,YZcv), (Xac,Cf,YZcw),(Xac,Cf,YZcx),(Xac,Cf,YZcy),(Xac,Cf,YZcz),(Xac,Cf,YZda),(Xac, Cf,YZdb),(Xac,Cf,YZdc),(Xac,Cf,YZdd),(Xac,Cf,YZde),(Xac,Cf,YZdf),(Xac,Cf,YZdg), (Xac,Cf,YZdh),(Xac,Cf,YZdi),(Xac,Cf,YZdj),(Xac,Cf,YZdk),(Xac,Cf,YZdl),(Xac, Cf,YZdm),(Xac,Cf,YZdn),(Xac,Cf,YZdo),(Xac,Cf,YZdp),(Xac,Cf,YZdq),(Xac,Cf,YZdr), (Xac,Cf,YZds),(Xac,Cf,Yzdt),(Xac,Cf,YZdu),(Xac,Cf,YZdv),(Xac,Cf,YZdw),(Xac, Cf,YZdx),(Xac,Cf,YZdy),(Xac,Cf,YZdz),(Xac,Cf,YZea),(Xac,Cf,YZeb),(Xac,Cf,YZec), (Xac,Cf,YZed),(Xac,Cf,YZee),(Xac,Cf,YZef),(Xac,Cf,YZeg),(Xac,Cf,YZeh),(Xac, Cf,YZei),(Xac,Cf,YZej),(Xac,Cf,YZek),(Xac,Cf,YZel),(Xac,Cf,YZem),(Xac,Cf,YZen), (Xac,Cf,YZeo),(Xac,Cf,YZep),(Xac,Cf,YZeq),(Xac,Cf,YZer),(Xac,Cf,YZes),(Xac,Cf, YZet),(Xac,Cf,YZeu),(Xac,Cf,YZev),(Xac,Cf,YZew),(Xac,Cf,YZex),(Xac,Cf,YZey),(Xac, Cf,YZez),(Xac,Cf,YZfa),(Xac,Cf,YZfb),(Xac,Cf,YZfc),(Xac,Cf,YZfd),(Xac,Cf,YZfe), (Xac,Cf,YZff),(Xac,Cf,YZfg),(Xac,Cf,YZfh),(Xac,Cf,YZfi),(Xac,Cf,YZfj),(Xac, Cf,YZfk),(Xac,Cf,YZfl),(Xac,Cf,YZfm),(Xac,Cf,YZfn),(Xac,Cf,YZfo),(Xac,Cf,YZfp), (Xac,Cf,YZfq),(Xac,Cf,YZfr),(Xac,Cf,YZfs),(Xac,Cf,YZft),(Xac,Cf,YZfu),(Xac,Cf,YZfv), (Xac,Cf,YZfw),(Xac,Cf,YZfx),(Xac,Cf,YZfy),(Xac,Cf,YZfz),(Xac,Cf,YZga),(Xac, Cf,YZgb),(Xac,Cf,YZgc),(Xac,Cf,YZgd),(Xac,Cf,YZge),(Xac,Cf,YZgf),(Xac,Cf,YZgg), (Xac,Cf,YZgh),(Xac,Cf,YZgi),(Xac,Cf,YZgj),(Xac,Cf,YZgk),(Xac,Cf,YZgl),(Xac, Cf,YZgm),(Xac,Cf,YZgn),(Xac,Cf,YZgo),(Xac,Cf,YZgp),(Xac,Cf,YZgq),(Xac,Cf,YZgr), (Xac,Cf,YZgs),(Xac,Cf,YZgt), (Xad,Ca,YZa),(Xad,Ca,YZb),(Xad,Ca,YZc),(Xad,Ca,YZd),(Xad,Ca,YZe),(Xad,Ca,YZf), (Xad,Ca,YZg),(Xad,Ca,YZh),(Xad,Ca,YZi),(Xad,Ca,YZj),(Xad,Ca,YZk),(Xad,Ca,YZl), (Xad,Ca,YZm),(Xad,Ca,YZn),(Xad,Ca,YZo),(Xad,Ca,YZp),(Xad,Ca,YZq),(Xad,Ca, YZr),(Xad,Ca,YZs),(Xad,Ca,YZt),(Xad,Ca,YZu),(Xad,Ca,YZv),(Xad,Ca,YZw),(Xad, Ca,YZx),(Xad,Ca,YZy),(Xad,Ca,YZz),(Xad,Ca,YZaa),(Xad,Ca,YZab),(Xad,Ca,YZac), (Xad,Ca,YZad),(Xad,Ca,YZae),(Xad,Ca,YZaf),(Xad,Ca,YZag),(Xad,Ca,YZah),(Xad, Ca,YZai),(Xad,Ca,YZaj),(Xad,Ca,YZak),(Xad,Ca,YZal),(Xad,Ca,YZam),(Xad,Ca,YZan), (Xad,Ca,YZao),(Xad,Ca,YZap),(Xad,Ca,YZaq),(Xad,Ca,YZar),(Xad,Ca,YZas),(Xad, Ca,YZat),(Xad,Ca,YZau),(Xad,Ca,YZav),(Xad,Ca,YZaw),(Xad,Ca,YZax),(Xad,Ca, YZay),(Xad,Ca,YZaz),(Xad,Ca,YZba),(Xad,Ca,YZbb),(Xad,Ca,YZbc),(Xad,Ca,YZbd), (Xad,Ca,YZbe),(Xad,Ca,YZbf),(Xad,Ca,YZbg),(Xad,Ca,YZbh),(Xad,Ca,YZbi),(Xad, Ca,YZbj),(Xad,Ca,YZbk),(Xad,Ca,YZbl),(Xad,Ca,YZbm),(Xad,Ca,YZbn),(Xad,Ca,YZbo), (Xad,Ca,YZbp),(Xad,Ca,YZbq),(Xad,Ca,YZbr),(Xad,Ca,YZbs),(Xad,Ca,YZbt),(Xad, Ca,YZbu),(Xad,Ca,YZbv),(Xad,Ca,YZbw),(Xad,Ca,YZbx),(Xad,Ca,YZby),(Xad, Ca,YZbz),(Xad,Ca,YZca),(Xad,Ca,YZcb),(Xad,Ca,YZcc),(Xad,Ca,YZcd),(Xad,Ca,YZce), (Xad,Ca,YZcf),(Xad,Ca,YZcg),(Xad,Ca,YZch),(Xad,Ca,YZci),(Xad,Ca,YZcj),(Xad, Ca,YZck),(Xad,Ca,YZcl),(Xad,Ca,YZcm),(Xad,Ca,YZcn),(Xad,Ca,YZco),(Xad,Ca, YZcp),(Xad,Ca,YZcq),(Xad,Ca,YZcr),(Xad,Ca,YZcs),(Xad,Ca,YZct),(Xad,Ca,YZcu), (Xad,Ca,YZcv),(Xad,Ca,YZcw),(Xad,Ca,YZcx),(Xad,Ca,YZcy),(Xad,Ca,YZcz),(Xad, Ca,YZda),(Xad,Ca,YZdb),(Xad,Ca,YZdc),(Xad,Ca,YZdd),(Xad,Ca,YZde),(Xad,Ca,YZdf), (Xad,Ca,YZdg),(Xad,Ca,YZdh),(Xad,Ca,YZdi),(Xad,Ca,YZdj),(Xad,Ca,YZdk),(Xad, Ca,YZdl),(Xad,Ca,YZdm),(Xad,Ca,YZdn),(Xad,Ca,YZdo),(Xad,Ca,YZdp),(Xad, Ca,YZdq),(Xad,Ca,YZdr),(Xad,Ca,YZds),(Xad,Ca,Yzdt),(Xad,Ca,YZdu),(Xad,Ca,YZdv), (Xad,Ca,YZdw),(Xad,Ca,YZdx),(Xad,Ca,YZdy),(Xad,Ca,YZdz),(Xad,Ca,YZea),(Xad, Ca,YZeb),(Xad,Ca,YZec),(Xad,Ca,YZed),(Xad,Ca,YZee),(Xad,Ca,YZef),(Xad,Ca, YZeg),(Xad,Ca,YZeh),(Xad,Ca,YZei),(Xad,Ca,YZej),(Xad,Ca,YZek),(Xad,Ca,YZel), (Xad,Ca,YZem),(Xad,Ca,YZen),(Xad,Ca,YZeo),(Xad,Ca,YZep),(Xad,Ca,YZeq),(Xad, Ca,YZer),(Xad,Ca,YZes),(Xad,Ca,YZet),(Xad,Ca,YZeu),(Xad,Ca,YZev),(Xad,Ca,YZew), (Xad,Ca,YZex),(Xad,Ca,YZey),(Xad,Ca,YZez),(Xad,Ca,YZfa),(Xad,Ca,YZfb),(Xad, Ca,YZfc),(Xad,Ca,YZfd),(Xad,Ca,YZfe),(Xad,Ca,YZff),(Xad,Ca,YZfg),(Xad,Ca,YZfh), (Xad,Ca,YZfi),(Xad,Ca,YZfj),(Xad,Ca,YZfk),(Xad,Ca,YZfl),(Xad,Ca,YZfm),(Xad, Ca,YZfn),(Xad,Ca,YZfo),(Xad,Ca,YZfp),(Xad,Ca,YZfq),(Xad,Ca,YZfr),(Xad,Ca,YZfs), (Xad,Ca,YZft),(Xad,Ca,YZfu),(Xad,Ca,YZfv),(Xad,Ca,YZfw),(Xad,Ca,YZfx),(Xad, Ca,YZfy),(Xad,Ca,YZfz),(Xad,Ca,YZga),(Xad,Ca,YZgb),(Xad,Ca,YZgc),(Xad,Ca, YZgd),(Xad,Ca,YZge),(Xad,Ca,YZgf),(Xad,Ca,YZgg),(Xad,Ca,YZgh),(Xad,Ca,YZgi), (Xad,Ca,YZgj),(Xad,Ca,YZgk),(Xad,Ca,YZgl),(Xad,Ca,YZgm),(Xad,Ca,YZgn),(Xad, Ca,YZgo),(Xad,Ca,YZgp),(Xad,Ca,YZgq),(Xad,Ca,YZgr),(Xad,Ca,YZgs),(Xad,Ca,YZgt), (Xad,Cb,YZa),(Xad,Cb,YZb),(Xad,Cb,YZc),(Xad,Cb,YZd),(Xad,Cb,YZe),(Xad, Cb,YZf),(Xad,Cb,YZg),(Xad,Cb,YZh),(Xad,Cb,YZi),(Xad,Cb,YZj),(Xad,Cb,YZk),(Xad, Cb,YZl),(Xad,Cb,YZm),(Xad,Cb,YZn),(Xad,Cb,YZo),(Xad,Cb,YZp),(Xad,Cb,YZq), (Xad,Cb,YZr),(Xad,Cb,YZs),(Xad,Cb,YZt),(Xad,Cb,YZu),(Xad,Cb,YZv),(Xad,Cb,YZw), (Xad,Cb,YZx),(Xad,Cb,YZy),(Xad,Cb,YZz),(Xad,Cb,YZaa),(Xad,Cb,YZab),(Xad, Cb,YZac),(Xad,Cb,YZad),(Xad,Cb,YZae),(Xad,Cb,YZaf),(Xad,Cb,YZag),(Xad,Cb, YZah),(Xad,Cb,YZai),(Xad,Cb,YZaj),(Xad,Cb,YZak),(Xad,Cb,YZal),(Xad,Cb,YZam), (Xad,Cb,YZan),(Xad,Cb,YZao),(Xad,Cb,YZap),(Xad,Cb,YZaq),(Xad,Cb,YZar),(Xad, Cb,YZas),(Xad,Cb,YZat),(Xad,Cb,YZau),(Xad,Cb,YZav),(Xad,Cb,YZaw),(Xad,Cb,YZax), (Xad,Cb,YZay),(Xad,Cb,YZaz),(Xad,Cb,YZba),(Xad,Cb,YZbb),(Xad,Cb,YZbc), (Xad,Cb,YZbd),(Xad,Cb,YZbe),(Xad,Cb,YZbf),(Xad,Cb,YZbg),(Xad,Cb,YZbh),(Xad, Cb,YZbi),(Xad,Cb,YZbj),(Xad,Cb,YZbk),(Xad,Cb,YZbl),(Xad,Cb,YZbm),(Xad,Cb,YZbn), (Xad,Cb,YZbo),(Xad,Cb,YZbp),(Xad,Cb,YZbq),(Xad,Cb,YZbr),(Xad,Cb,YZbs), (Xad,Cb,YZbt),(Xad,Cb,YZbu),(Xad,Cb,YZbv),(Xad,Cb,YZbw),(Xad,Cb,YZbx),(Xad, Cb,YZby),(Xad,Cb,YZbz),(Xad,Cb,YZca),(Xad,Cb,YZcb),(Xad,Cb,YZcc),(Xad,Cb,YZcd), (Xad,Cb,YZce),(Xad,Cb,YZcf),(Xad,Cb,YZcg),(Xad,Cb,YZch),(Xad,Cb,YZci),(Xad, Cb,YZcj),(Xad,Cb,YZck),(Xad,Cb,YZcl),(Xad,Cb,YZcm),(Xad,Cb,YZcn),(Xad, Cb,YZco),(Xad,Cb,YZcp),(Xad,Cb,YZcq),(Xad,Cb,YZcr),(Xad,Cb,YZcs),(Xad,Cb,YZct), (Xad,Cb,YZcu),(Xad,Cb,YZcv),(Xad,Cb,YZcw),(Xad,Cb,YZcx),(Xad,Cb,YZcy), (Xad,Cb,YZcz),(Xad,Cb,YZda),(Xad,Cb,YZdb),(Xad,Cb,YZdc),(Xad,Cb,YZdd),(Xad, Cb,YZde),(Xad,Cb,YZdf),(Xad,Cb,YZdg),(Xad,Cb,YZdh),(Xad,Cb,YZdi),(Xad,Cb,YZdj), (Xad,Cb,YZdk),(Xad,Cb,YZdl),(Xad,Cb,YZdm),(Xad,Cb,YZdn),(Xad,Cb,YZdo), (Xad,Cb,YZdp),(Xad,Cb,YZdq),(Xad,Cb,YZdr),(Xad,Cb,YZds),(Xad,Cb,Yzdt),(Xad, Cb,YZdu),(Xad,Cb,YZdv),(Xad,Cb,YZdw),(Xad,Cb,YZdx),(Xad,Cb,YZdy),(Xad,Cb, YZdz),(Xad,Cb,YZea),(Xad,Cb,YZeb),(Xad,Cb,YZec),(Xad,Cb,YZed),(Xad,Cb,YZee), (Xad,Cb,YZef),(Xad,Cb,YZeg),(Xad,Cb,YZeh),(Xad,Cb,YZei),(Xad,Cb,YZej),(Xad, Cb,YZek),(Xad,Cb,YZel),(Xad,Cb,YZem),(Xad,Cb,YZen),(Xad,Cb,YZeo),(Xad,Cb,YZep), (Xad,Cb,YZeq),(Xad,Cb,YZer),(Xad,Cb,YZes),(Xad,Cb,YZet),(Xad,Cb,YZeu),(Xad, Cb,YZev),(Xad,Cb,YZew),(Xad,Cb,YZex),(Xad,Cb,YZey),(Xad,Cb,YZez),(Xad, Cb,YZfa),(Xad,Cb,YZfb),(Xad,Cb,YZfc),(Xad,Cb,YZfd),(Xad,Cb,YZfe),(Xad,Cb,YZff), (Xad,Cb,YZfg),(Xad,Cb,YZfh),(Xad,Cb,YZfi),(Xad,Cb,YZfj),(Xad,Cb,YZfk),(Xad, Cb,YZfl),(Xad,Cb,YZfm),(Xad,Cb,YZfn),(Xad,Cb,YZfo),(Xad,Cb,YZfp),(Xad,Cb,YZfq), (Xad,Cb,YZfr),(Xad,Cb,YZfs),(Xad,Cb,YZft),(Xad,Cb,YZfu),(Xad,Cb,YZfv),(Xad, Cb,YZfw),(Xad,Cb,YZfx),(Xad,Cb,YZfy),(Xad,Cb,YZfz),(Xad,Cb,YZga),(Xad,Cb,YZgb), (Xad,Cb,YZgc),(Xad,Cb,YZgd),(Xad,Cb,YZge),(Xad,Cb,YZgf),(Xad,Cb,YZgg), (Xad,Cb,YZgh),(Xad,Cb,YZgi),(Xad,Cb,YZgj),(Xad,Cb,YZgk),(Xad,Cb,YZgl),(Xad, Cb,YZgm),(Xad,Cb,YZgn),(Xad,Cb,YZgo),(Xad,Cb,YZgp),(Xad,Cb,YZgq),(Xad,Cb, YZgr),(Xad,Cb,YZgs),(Xad,Cb,YZgt),(Xad,Cc,YZa),(Xad,Cc,YZb),(Xad,Cc,YZc),(Xad, Cc,YZd),(Xad,Cc,YZe),(Xad,Cc,YZf),(Xad,Cc,YZg),(Xad,Cc,YZh),(Xad,Cc,YZi),(Xad, Cc,YZj),(Xad,Cc,YZk),(Xad,Cc,YZl),(Xad,Cc,YZm),(Xad,Cc,YZn),(Xad,Cc,YZo), (Xad,Cc,YZp),(Xad,Cc,YZq),(Xad,Cc,YZr),(Xad,Cc,YZs),(Xad,Cc,YZt),(Xad,Cc,YZu), (Xad,Cc,YZv),(Xad,Cc,YZw),(Xad,Cc,YZx),(Xad,Cc,YZy),(Xad,Cc,YZz),(Xad, Cc,YZaa),(Xad,Cc,YZab),(Xad,Cc,YZac),(Xad,Cc,YZad),(Xad,Cc,YZae),(Xad,Cc,YZaf), (Xad,Cc,YZag),(Xad,Cc,YZah),(Xad,Cc,YZai),(Xad,Cc,YZaj),(Xad,Cc,YZak),(Xad, Cc,YZal),(Xad,Cc,YZam),(Xad,Cc,YZan),(Xad,Cc,YZao),(Xad,Cc,YZap),(Xad,Cc, YZaq),(Xad,Cc,YZar),(Xad,Cc,YZas),(Xad,Cc,YZat),(Xad,Cc,YZau),(Xad,Cc,YZav), (Xad,Cc,YZaw),(Xad,Cc,YZax),(Xad,Cc,YZay),(Xad,Cc,YZaz),(Xad,Cc,YZba),(Xad, Cc,YZbb),(Xad,Cc,YZbc),(Xad,Cc,YZbd),(Xad,Cc,YZbe),(Xad,Cc,YZbf),(Xad,Cc,YZbg), (Xad,Cc,YZbh),(Xad,Cc,YZbi),(Xad,Cc,YZbj),(Xad,Cc,YZbk),(Xad,Cc,YZbl),(Xad, Cc,YZbm),(Xad,Cc,YZbn),(Xad,Cc,YZbo),(Xad,Cc,YZbp),(Xad,Cc,YZbq),(Xad, Cc,YZbr),(Xad,Cc,YZbs),(Xad,Cc,YZbt),(Xad,Cc,YZbu),(Xad,Cc,YZbv),(Xad,Cc,YZbw), (Xad,Cc,YZbx),(Xad,Cc,YZby),(Xad,Cc,YZbz),(Xad,Cc,YZca),(Xad,Cc,YZcb),(Xad, Cc,YZcc),(Xad,Cc,YZcd),(Xad,Cc,YZce),(Xad,Cc,YZcf),(Xad,Cc,YZcg),(Xad,Cc, YZch),(Xad,Cc,YZci),(Xad,Cc,YZcj),(Xad,Cc,YZck),(Xad,Cc,YZcl),(Xad,Cc,YZcm), (Xad,Cc,YZcn),(Xad,Cc,YZco),(Xad,Cc,YZcp),(Xad,Cc,YZcq),(Xad,Cc,YZcr),(Xad, Cc,YZcs),(Xad,Cc,YZct),(Xad,Cc,YZcu),(Xad,Cc,YZcv),(Xad,Cc,YZcw),(Xad,Cc,YZcx), (Xad,Cc,YZcy),(Xad,Cc,YZcz),(Xad,Cc,YZda),(Xad,Cc,YZdb),(Xad,Cc,YZdc),(Xad, Cc,YZdd),(Xad,Cc,YZde),(Xad,Cc,YZdf),(Xad,Cc,YZdg),(Xad,Cc,YZdh),(Xad,Cc, YZdi),(Xad,Cc,YZdj),(Xad,Cc,YZdk),(Xad,Cc,YZdl),(Xad,Cc,YZdm),(Xad,Cc,YZdn), (Xad,Cc,YZdo),(Xad,Cc,YZdp),(Xad,Cc,YZdq),(Xad,Cc,YZdr),(Xad,Cc,YZds),(Xad, Cc,Yzdt),(Xad,Cc,YZdu),(Xad,Cc,YZdv),(Xad,Cc,YZdw),(Xad,Cc,YZdx),(Xad,Cc, YZdy),(Xad,Cc,YZdz),(Xad,Cc,YZea),(Xad,Cc,YZeb),(Xad,Cc,YZec),(Xad,Cc,YZed), (Xad,Cc,YZee),(Xad,Cc,YZef),(Xad,Cc,YZeg),(Xad,Cc,YZeh),(Xad,Cc,YZei),(Xad, Cc,YZej),(Xad,Cc,YZek),(Xad,Cc,YZel),(Xad,Cc,YZem),(Xad,Cc,YZen),(Xad,Cc,YZeo), (Xad,Cc,YZep),(Xad,Cc,YZeq),(Xad,Cc,YZer),(Xad,Cc,YZes),(Xad,Cc,YZet),(Xad, Cc,YZeu),(Xad,Cc,YZev),(Xad,Cc,YZew),(Xad,Cc,YZex),(Xad,Cc,YZey),(Xad,Cc, YZez),(Xad,Cc,YZfa),(Xad,Cc,YZfb),(Xad,Cc,YZfc),(Xad,Cc,YZfd),(Xad,Cc,YZfe),(Xad, Cc,YZff),(Xad,Cc,YZfg),(Xad,Cc,YZfh),(Xad,Cc,YZfi),(Xad,Cc,YZfj),(Xad,Cc, YZfk),(Xad,Cc,YZfl),(Xad,Cc,YZfm),(Xad,Cc,YZfn),(Xad,Cc,YZfo),(Xad,Cc,YZfp),(Xad, Cc,YZfq),(Xad,Cc,YZfr),(Xad,Cc,YZfs),(Xad,Cc,YZft),(Xad,Cc,YZfu),(Xad,Cc, YZfv),(Xad,Cc,YZfw),(Xad,Cc,YZfx),(Xad,Cc,YZfy),(Xad,Cc,YZfz),(Xad,Cc,YZga), (Xad,Cc,YZgb),(Xad,Cc,YZgc),(Xad,Cc,YZgd),(Xad,Cc,YZge),(Xad,Cc,YZgf),(Xad, Cc,YZgg),(Xad,Cc,YZgh),(Xad,Cc,YZgi),(Xad,Cc,YZgj),(Xad,Cc,YZgk),(Xad,Cc,YZgl), (Xad,Cc,YZgm),(Xad,Cc,YZgn),(Xad,Cc,YZgo),(Xad,Cc,YZgp),(Xad,Cc,YZgq),(Xad, Cc,YZgr),(Xad,Cc,YZgs),(Xad,Cc,YZgt),(Xad,Cd,YZa),(Xad,Cd,YZb),(Xad,Cd, YZc),(Xad,Cd,YZd),(Xad,Cd,YZe),(Xad,Cd,YZf),(Xad,Cd,YZg),(Xad,Cd,YZh),(Xad, Cd,YZi),(Xad,Cd,YZj),(Xad,Cd,YZk),(Xad,Cd,YZl),(Xad,Cd,YZm),(Xad,Cd,YZn),(Xad, Cd,YZo),(Xad,Cd,YZp),(Xad,Cd,YZq),(Xad,Cd,YZr),(Xad,Cd,YZs),(Xad,Cd,YZt), (Xad,Cd,YZu),(Xad,Cd,YZv),(Xad,Cd,YZw),(Xad,Cd,YZx),(Xad,Cd,YZy),(Xad,Cd, YZz),(Xad,Cd,YZaa),(Xad,Cd,YZab),(Xad,Cd,YZac),(Xad,Cd,YZad),(Xad,Cd,YZae), (Xad,Cd,YZaf),(Xad,Cd,YZag),(Xad,Cd,YZah),(Xad,Cd,YZai),(Xad,Cd,YZaj),(Xad,Cd, YZak),(Xad,Cd,YZal),(Xad,Cd,YZam),(Xad,Cd,YZan),(Xad,Cd,YZao),(Xad,Cd,YZap), (Xad,Cd,YZaq),(Xad,Cd,YZar),(Xad,Cd,YZas),(Xad,Cd,YZat),(Xad,Cd,YZau),(Xad, Cd,YZav),(Xad,Cd,YZaw),(Xad,Cd,YZax),(Xad,Cd,YZay),(Xad,Cd,YZaz),(Xad,Cd, YZba),(Xad,Cd,YZbb),(Xad,Cd,YZbc),(Xad,Cd,YZbd),(Xad,Cd,YZbe),(Xad,Cd,YZbf), (Xad,Cd,YZbg),(Xad,Cd,YZbh),(Xad,Cd,YZbi),(Xad,Cd,YZbj),(Xad,Cd,YZbk),(Xad, Cd,YZbl),(Xad,Cd,YZbm),(Xad,Cd,YZbn),(Xad,Cd,YZbo),(Xad,Cd,YZbp),(Xad,Cd, YZbq),(Xad,Cd,YZbr),(Xad,Cd,YZbs),(Xad,Cd,YZbt),(Xad,Cd,YZbu),(Xad,Cd,YZbv), (Xad,Cd,YZbw),(Xad,Cd,YZbx),(Xad,Cd,YZby),(Xad,Cd,YZbz),(Xad,Cd,YZca),(Xad, Cd,YZcb),(Xad,Cd,YZcc),(Xad,Cd,YZcd),(Xad,Cd,YZce),(Xad,Cd,YZcf),(Xad,Cd, YZcg),(Xad,Cd,YZch),(Xad,Cd,YZci),(Xad,Cd,YZcj),(Xad,Cd,YZck),(Xad,Cd,YZcl), (Xad,Cd,YZcm),(Xad,Cd,YZcn),(Xad,Cd,YZco),(Xad,Cd,YZcp),(Xad,Cd,YZcq),(Xad, Cd,YZcr),(Xad,Cd,YZcs),(Xad,Cd,YZct),(Xad,Cd,YZcu),(Xad,Cd,YZcv),(Xad,Cd, YZcw),(Xad,Cd,YZcx),(Xad,Cd,YZcy),(Xad,Cd,YZcz),(Xad,Cd,YZda),(Xad,Cd,YZdb), (Xad,Cd,YZdc),(Xad,Cd,YZdd),(Xad,Cd,YZde),(Xad,Cd,YZdf),(Xad,Cd,YZdg),(Xad, Cd,YZdh),(Xad,Cd,YZdi),(Xad,Cd,YZdj),(Xad,Cd,YZdk),(Xad,Cd,YZdl),(Xad,Cd, YZdm),(Xad,Cd,YZdn),(Xad,Cd,YZdo),(Xad,Cd,YZdp),(Xad,Cd,YZdq),(Xad,Cd,YZdr), (Xad,Cd,YZds),(Xad,Cd,Yzdt),(Xad,Cd,YZdu),(Xad,Cd,YZdv),(Xad,Cd,YZdw),(Xad, Cd,YZdx),(Xad,Cd,YZdy),(Xad,Cd,YZdz),(Xad,Cd,YZea),(Xad,Cd,YZeb),(Xad, Cd,YZec),(Xad,Cd,YZed),(Xad,Cd,YZee),(Xad,Cd,YZef),(Xad,Cd,YZeg),(Xad,Cd,YZeh), (Xad,Cd,YZei),(Xad,Cd,YZej),(Xad,Cd,YZek),(Xad,Cd,YZel),(Xad,Cd,YZem),(Xad, Cd,YZen),(Xad,Cd,YZeo),(Xad,Cd,YZep),(Xad,Cd,YZeq),(Xad,Cd,YZer),(Xad, Cd,YZes),(Xad,Cd,YZet),(Xad,Cd,YZeu),(Xad,Cd,YZev),(Xad,Cd,YZew),(Xad,Cd,YZex), (Xad,Cd,YZey),(Xad,Cd,YZez),(Xad,Cd,YZfa),(Xad,Cd,YZfb),(Xad,Cd,YZfc),(Xad, Cd,YZfd),(Xad,Cd,YZfe),(Xad,Cd,YZff),(Xad,Cd,YZfg),(Xad,Cd,YZfh),(Xad,Cd, YZfi),(Xad,Cd,YZfj),(Xad,Cd,YZfk),(Xad,Cd,YZfl),(Xad,Cd,YZfm),(Xad,Cd,YZfn), (Xad,Cd,YZfo),(Xad,Cd,YZfp),(Xad,Cd,YZfq),(Xad,Cd,YZfr),(Xad,Cd,YZfs),(Xad,Cd, YZft),(Xad,Cd,YZfu),(Xad,Cd,YZfv),(Xad,Cd,YZfw),(Xad,Cd,YZfx),(Xad,Cd,YZfy), (Xad,Cd,YZfz),(Xad,Cd,YZga),(Xad,Cd,YZgb),(Xad,Cd,YZgc),(Xad,Cd,YZgd),(Xad, Cd,YZge),(Xad,Cd,YZgf),(Xad,Cd,YZgg),(Xad,Cd,YZgh),(Xad,Cd,YZgi),(Xad,Cd, YZgj),(Xad,Cd,YZgk),(Xad,Cd,YZgl),(Xad,Cd,YZgm),(Xad,Cd,YZgn),(Xad,Cd,YZgo), (Xad,Cd,YZgp),(Xad,Cd,YZgq),(Xad,Cd,YZgr),(Xad,Cd,YZgs),(Xad,Cd,YZgt),(Xad, Ce,YZa),(Xad,Ce,YZb),(Xad,Ce,YZc),(Xad,Ce,YZd),(Xad,Ce,YZe),(Xad,Ce,YZf),(Xad, Ce,YZg),(Xad,Ce,YZh),(Xad,Ce,YZi),(Xad,Ce,YZj),(Xad,Ce,YZk),(Xad,Ce,YZl), (Xad,Ce,YZm),(Xad,Ce,YZn),(Xad,Ce,YZo),(Xad,Ce,YZp),(Xad,Ce,YZq),(Xad,Ce,YZr), (Xad,Ce,YZs),(Xad,Ce,YZt),(Xad,Ce,YZu),(Xad,Ce,YZv),(Xad,Ce,YZw),(Xad,Ce, YZx),(Xad,Ce,YZy),(Xad,Ce,YZz),(Xad,Ce,YZaa),(Xad,Ce,YZab),(Xad,Ce,YZac),(Xad, Ce,YZad),(Xad,Ce,YZae),(Xad,Ce,YZaf),(Xad,Ce,YZag),(Xad,Ce,YZah),(Xad,Ce, YZai),(Xad,Ce,YZaj),(Xad,Ce,YZak),(Xad,Ce,YZal),(Xad,Ce,YZam),(Xad,Ce,YZan), (Xad,Ce,YZao),(Xad,Ce,YZap),(Xad,Ce,YZaq),(Xad,Ce,YZar),(Xad,Ce,YZas),(Xad, Ce,YZat),(Xad,Ce,YZau),(Xad,Ce,YZav),(Xad,Ce,YZaw),(Xad,Ce,YZax),(Xad,Ce,YZay), (Xad,Ce,YZaz),(Xad,Ce,YZba),(Xad,Ce,YZbb),(Xad,Ce,YZbc),(Xad,Ce,YZbd),(Xad, Ce,YZbe),(Xad,Ce,YZbf),(Xad,Ce,YZbg),(Xad,Ce,YZbh),(Xad,Ce,YZbi),(Xad,Ce, YZbj),(Xad,Ce,YZbk),(Xad,Ce,YZbl),(Xad,Ce,YZbm),(Xad,Ce,YZbn),(Xad,Ce,YZbo), (Xad,Ce,YZbp),(Xad,Ce,YZbq),(Xad,Ce,YZbr),(Xad,Ce,YZbs),(Xad,Ce,YZbt),(Xad, Ce,YZbu),(Xad,Ce,YZbv),(Xad,Ce,YZbw),(Xad,Ce,YZbx),(Xad,Ce,YZby),(Xad,Ce, YZbz),(Xad,Ce,YZca),(Xad,Ce,YZcb),(Xad,Ce,YZcc),(Xad,Ce,YZcd),(Xad,Ce,YZce), (Xad,Ce,YZcf),(Xad,Ce,YZcg),(Xad,Ce,YZch),(Xad,Ce,YZci),(Xad,Ce,YZcj),(Xad,Ce, YZck),(Xad,Ce,YZcl),(Xad,Ce,YZcm),(Xad,Ce,YZcn),(Xad,Ce,YZco),(Xad,Ce,YZcp), (Xad,Ce,YZcq),(Xad,Ce,YZcr),(Xad,Ce,YZcs),(Xad,Ce,YZct),(Xad,Ce,YZcu),(Xad, Ce,YZcv),(Xad,Ce,YZcw),(Xad,Ce,YZcx),(Xad,Ce,YZcy),(Xad,Ce,YZcz),(Xad,Ce,YZda), (Xad,Ce,YZdb),(Xad,Ce,YZdc),(Xad,Ce,YZdd),(Xad,Ce,YZde),(Xad,Ce,YZdf),(Xad, Ce,YZdg),(Xad,Ce,YZdh),(Xad,Ce,YZdi),(Xad,Ce,YZdj),(Xad,Ce,YZdk),(Xad,Ce, YZdl),(Xad,Ce,YZdm),(Xad,Ce,YZdn),(Xad,Ce,YZdo),(Xad,Ce,YZdp),(Xad,Ce,YZdq), (Xad,Ce,YZdr),(Xad,Ce,YZds),(Xad,Ce,Yzdt),(Xad,Ce,YZdu),(Xad,Ce,YZdv),(Xad, Ce,YZdw),(Xad,Ce,YZdx),(Xad,Ce,YZdy),(Xad,Ce,YZdz),(Xad,Ce,YZea),(Xad,Ce, YZeb),(Xad,Ce,YZec),(Xad,Ce,YZed),(Xad,Ce,YZee),(Xad,Ce,YZef),(Xad,Ce,YZeg), (Xad,Ce,YZeh),(Xad,Ce,YZei),(Xad,Ce,YZej),(Xad,Ce,YZek),(Xad,Ce,YZel),(Xad,Ce, YZem),(Xad,Ce,YZen),(Xad,Ce,YZeo),(Xad,Ce,YZep),(Xad,Ce,YZeq),(Xad,Ce,YZer), (Xad,Ce,YZes),(Xad,Ce,YZet),(Xad,Ce,YZeu),(Xad,Ce,YZev),(Xad,Ce,YZew),(Xad, Ce,YZex),(Xad,Ce,YZey),(Xad,Ce,YZez),(Xad,Ce,YZfa),(Xad,Ce,YZfb),(Xad,Ce, YZfc),(Xad,Ce,YZfd),(Xad,Ce,YZfe),(Xad,Ce,YZff),(Xad,Ce,YZfg),(Xad,Ce,YZfh),(Xad, Ce,YZfi),(Xad,Ce,YZfj),(Xad,Ce,YZfk),(Xad,Ce,YZfl),(Xad,Ce,YZfm),(Xad,Ce, YZfn),(Xad,Ce,YZfo),(Xad,Ce,YZfp),(Xad,Ce,YZfq),(Xad,Ce,YZfr),(Xad,Ce,YZfs),(Xad, Ce,YZft),(Xad,Ce,YZfu),(Xad,Ce,YZfv),(Xad,Ce,YZfw),(Xad,Ce,YZfx),(Xad,Ce, YZfy),(Xad,Ce,YZfz),(Xad,Ce,YZga),(Xad,Ce,YZgb),(Xad,Ce,YZgc),(Xad,Ce,YZgd), (Xad,Ce,YZge),(Xad,Ce,YZgf),(Xad,Ce,YZgg),(Xad,Ce,YZgh),(Xad,Ce,YZgi),(Xad, Ce,YZgj),(Xad,Ce,YZgk),(Xad,Ce,YZgl),(Xad,Ce,YZgm),(Xad,Ce,YZgn),(Xad,Ce,YZgo), (Xad,Ce,YZgp),(Xad,Ce,YZgq),(Xad,Ce,YZgr),(Xad,Ce,YZgs),(Xad,Ce,YZgt),(Xad, Cf,YZa),(Xad,Cf,YZb),(Xad,Cf,YZc),(Xad,Cf,YZd),(Xad,Cf,YZe),(Xad,Cf,YZf), (Xad,Cf,YZg),(Xad,Cf,YZh),(Xad,Cf,YZi),(Xad,Cf,YZj),(Xad,Cf,YZk),(Xad,Cf,YZl), (Xad,Cf,YZm),(Xad,Cf,YZn),(Xad,Cf,YZo),(Xad,Cf,YZp),(Xad,Cf,YZq),(Xad,Cf,YZr), (Xad,Cf,YZs),(Xad,Cf,YZt),(Xad,Cf,YZu),(Xad,Cf,YZv),(Xad,Cf,YZw),(Xad,Cf,YZx), (Xad,Cf,YZy),(Xad,Cf,YZz),(Xad,Cf,YZaa),(Xad,Cf,YZab),(Xad,Cf,YZac),(Xad, Cf,YZad),(Xad,Cf,YZae),(Xad,Cf,YZaf),(Xad,Cf,YZag),(Xad,Cf,YZah),(Xad,Cf,YZai), (Xad,Cf,YZaj),(Xad,Cf,YZak),(Xad,Cf,YZal),(Xad,Cf,YZam),(Xad,Cf,YZan),(Xad, Cf,YZao),(Xad,Cf,YZap),(Xad,Cf,YZaq),(Xad,Cf,YZar),(Xad,Cf,YZas),(Xad,Cf,YZat), (Xad,Cf,YZau),(Xad,Cf,YZav),(Xad,Cf,YZaw),(Xad,Cf,YZax),(Xad,Cf,YZay),(Xad, Cf,YZaz),(Xad,Cf,YZba),(Xad,Cf,YZbb),(Xad,Cf,YZbc),(Xad,Cf,YZbd),(Xad,Cf,YZbe), (Xad,Cf,YZbf),(Xad,Cf,YZbg),(Xad,Cf,YZbh),(Xad,Cf,YZbi),(Xad,Cf,YZbj),(Xad, Cf,YZbk),(Xad,Cf,YZbl),(Xad,Cf,YZbm),(Xad,Cf,YZbn),(Xad,Cf,YZbo),(Xad,Cf,YZbp), (Xad,Cf,YZbq),(Xad,Cf,YZbr),(Xad,Cf,YZbs),(Xad,Cf,YZbt),(Xad,Cf,YZbu),(Xad, Cf,YZbv),(Xad,Cf,YZbw),(Xad,Cf,YZbx),(Xad,Cf,YZby),(Xad,Cf,YZbz),(Xad,Cf,YZca), (Xad,Cf,YZcb),(Xad,Cf,YZcc),(Xad,Cf,YZcd),(Xad,Cf,YZce),(Xad,Cf,YZcf),(Xad, Cf,YZcg),(Xad,Cf,YZch),(Xad,Cf,YZci),(Xad,Cf,YZcj),(Xad,Cf,YZck),(Xad,Cf,YZcl), (Xad,Cf,YZcm),(Xad,Cf,YZcn),(Xad,Cf,YZco),(Xad,Cf,YZcp),(Xad,Cf,YZcq),(Xad, Cf,YZcr),(Xad,Cf,YZcs),(Xad,Cf,YZct),(Xad,Cf,YZcu),(Xad,Cf,YZcv),(Xad,Cf, YZcw),(Xad,Cf,YZcx),(Xad,Cf,YZcy),(Xad,Cf,YZcz),(Xad,Cf,YZda),(Xad,Cf,YZdb), (Xad,Cf,YZdc),(Xad,Cf,YZdd),(Xad,Cf,YZde),(Xad,Cf,YZdf),(Xad,Cf,YZdg),(Xad,Cf, YZdh),(Xad,Cf,YZdi),(Xad,Cf,YZdj),(Xad,Cf,YZdk),(Xad,Cf,YZdl),(Xad,Cf,YZdm), (Xad,Cf,YZdn),(Xad,Cf,YZdo),(Xad,Cf,YZdp),(Xad,Cf,YZdq),(Xad,Cf,YZdr),(Xad, Cf,YZds),(Xad,Cf,Yzdt),(Xad,Cf,YZdu),(Xad,Cf,YZdv),(Xad,Cf,YZdw),(Xad,Cf,YZdx), (Xad,Cf,YZdy),(Xad,Cf,YZdz),(Xad,Cf,YZea),(Xad,Cf,YZeb),(Xad,Cf,YZec),(Xad, Cf,YZed),(Xad,Cf,YZee),(Xad,Cf,YZef),(Xad,Cf,YZeg),(Xad,Cf,YZeh),(Xad,Cf,YZei), (Xad,Cf,YZej),(Xad,Cf,YZek),(Xad,Cf,YZel),(Xad,Cf,YZem),(Xad,Cf,YZen),(Xad, Cf,YZeo),(Xad,Cf,YZep),(Xad,Cf,YZeq),(Xad,Cf,YZer),(Xad,Cf,YZes),(Xad,Cf,YZet), (Xad,Cf,YZeu),(Xad,Cf,YZev),(Xad,Cf,YZew),(Xad,Cf,YZex),(Xad,Cf,YZey),(Xad, Cf,YZez),(Xad,Cf,YZfa),(Xad,Cf,YZfb),(Xad,Cf,YZfc),(Xad,Cf,YZfd),(Xad,Cf,YZfe), (Xad,Cf,YZff),(Xad,Cf,YZfg),(Xad,Cf,YZfh),(Xad,Cf,YZfi),(Xad,Cf,YZfj),(Xad,Cf, YZfk),(Xad,Cf,YZfl),(Xad,Cf,YZfm),(Xad,Cf,YZfn),(Xad,Cf,YZfo),(Xad,Cf,YZfp),(Xad, Cf,YZfq),(Xad,Cf,YZfr),(Xad,Cf,YZfs),(Xad,Cf,YZft),(Xad,Cf,YZfu),(Xad,Cf,YZfv), (Xad,Cf,YZfw),(Xad,Cf,YZfx),(Xad,Cf,YZfy),(Xad,Cf,YZfz),(Xad,Cf,YZga),(Xad, Cf,YZgb),(Xad,Cf,YZgc),(Xad,Cf,YZgd),(Xad,Cf,YZge),(Xad,Cf,YZgf),(Xad,Cf,YZgg), (Xad,Cf,YZgh),(Xad,Cf,YZgi),(Xad,Cf,YZgj),(Xad,Cf,YZgk),(Xad,Cf,YZgl),(Xad, Cf,YZgm),(Xad,Cf,YZgn),(Xad,Cf,YZgo),(Xad,Cf,YZgp),(Xad,Cf,YZgq),(Xad,Cf, YZgr),(Xad,Cf,YZgs),(Xad,Cf,YZgt), (Xae,Ca,YZa),(Xae,Ca,YZb),(Xae,Ca,YZc),(Xae,Ca,YZd),(Xae,Ca,YZe),(Xae,Ca,YZf), (Xae,Ca,YZg),(Xae,Ca,YZh),(Xae,Ca,YZi),(Xae,Ca,YZj),(Xae,Ca,YZk),(Xae,Ca,YZl), (Xae,Ca,YZm),(Xae,Ca,YZn),(Xae,Ca,YZo),(Xae,Ca,YZp),(Xae,Ca,YZq),(Xae,Ca, YZr),(Xae,Ca,YZs),(Xae,Ca,YZt),(Xae,Ca,YZu),(Xae,Ca,YZv),(Xae,Ca,YZw),(Xae,Ca, YZx),(Xae,Ca,YZy),(Xae,Ca,YZz),(Xae,Ca,YZaa),(Xae,Ca,YZab),(Xae,Ca,YZac),(Xae, Ca,YZad),(Xae,Ca,YZae),(Xae,Ca,YZaf),(Xae,Ca,YZag),(Xae,Ca,YZah),(Xae,Ca, YZai),(Xae,Ca,YZaj),(Xae,Ca,YZak),(Xae,Ca,YZal),(Xae,Ca,YZam),(Xae,Ca,YZan), (Xae,Ca,YZao),(Xae,Ca,YZap),(Xae,Ca,YZaq),(Xae,Ca,YZar),(Xae,Ca,YZas),(Xae,Ca, YZat),(Xae,Ca,YZau),(Xae,Ca,YZav),(Xae,Ca,YZaw),(Xae,Ca,YZax),(Xae,Ca,YZay), (Xae,Ca,YZaz),(Xae,Ca,YZba),(Xae,Ca,YZbb),(Xae,Ca,YZbc),(Xae,Ca,YZbd),(Xae, Ca,YZbe),(Xae,Ca,YZbf),(Xae,Ca,YZbg),(Xae,Ca,YZbh),(Xae,Ca,YZbi),(Xae,Ca,YZbj), (Xae,Ca,YZbk),(Xae,Ca,YZbl),(Xae,Ca,YZbm),(Xae,Ca,YZbn),(Xae,Ca,YZbo),(Xae, Ca,YZbp),(Xae,Ca,YZbq),(Xae,Ca,YZbr),(Xae,Ca,YZbs),(Xae,Ca,YZbt),(Xae,Ca, YZbu),(Xae,Ca,YZbv),(Xae,Ca,YZbw),(Xae,Ca,YZbx),(Xae,Ca,YZby),(Xae,Ca,YZbz), (Xae,Ca,YZca),(Xae,Ca,YZcb),(Xae,Ca,YZcc),(Xae,Ca,YZcd),(Xae,Ca,YZce),(Xae, Ca,YZcf),(Xae,Ca,YZcg),(Xae,Ca,YZch),(Xae,Ca,YZci),(Xae,Ca,YZcj),(Xae,Ca,YZck), (Xae,Ca,YZcl),(Xae,Ca,YZcm),(Xae,Ca,YZcn),(Xae,Ca,YZco),(Xae,Ca,YZcp),(Xae, Ca,YZcq),(Xae,Ca,YZcr),(Xae,Ca,YZcs),(Xae,Ca,YZct),(Xae,Ca,YZcu),(Xae,Ca,YZcv), (Xae,Ca,YZcw),(Xae,Ca,YZcx),(Xae,Ca,YZcy),(Xae,Ca,YZcz),(Xae,Ca,YZda),(Xae, Ca,YZdb),(Xae,Ca,YZdc),(Xae,Ca,YZdd),(Xae,Ca,YZde),(Xae,Ca,YZdf),(Xae,Ca, YZdg),(Xae,Ca,YZdh),(Xae,Ca,YZdi),(Xae,Ca,YZdj),(Xae,Ca,YZdk),(Xae,Ca,YZdl),(Xae, Ca,YZdm),(Xae,Ca,YZdn),(Xae,Ca,YZdo),(Xae,Ca,YZdp),(Xae,Ca,YZdq),(Xae, Ca,YZdr),(Xae,Ca,YZds),(Xae,Ca,Yzdt),(Xae,Ca,YZdu),(Xae,Ca,YZdv),(Xae,Ca,YZdw), (Xae,Ca,YZdx),(Xae,Ca,YZdy),(Xae,Ca,YZdz),(Xae,Ca,YZea),(Xae,Ca,YZeb),(Xae, Ca,YZec),(Xae,Ca,YZed),(Xae,Ca,YZee),(Xae,Ca,YZef),(Xae,Ca,YZeg),(Xae,Ca,YZeh), (Xae,Ca,YZei),(Xae,Ca,YZej),(Xae,Ca,YZek),(Xae,Ca,YZel),(Xae,Ca,YZem),(Xae, Ca,YZen),(Xae,Ca,YZeo),(Xae,Ca,YZep),(Xae,Ca,YZeq),(Xae,Ca,YZer),(Xae,Ca, YZes),(Xae,Ca,YZet),(Xae,Ca,YZeu),(Xae,Ca,YZev),(Xae,Ca,YZew),(Xae,Ca,YZex), (Xae,Ca,YZey),(Xae,Ca,YZez),(Xae,Ca,YZfa),(Xae,Ca,YZfb),(Xae,Ca,YZfc),(Xae,Ca, YZfd),(Xae,Ca,YZfe),(Xae,Ca,YZff),(Xae,Ca,YZfg),(Xae,Ca,YZfh),(Xae,Ca,YZfi),(Xae, Ca,YZfj),(Xae,Ca,YZfk),(Xae,Ca,YZfl),(Xae,Ca,YZfm),(Xae,Ca,YZfn),(Xae,Ca, YZfo),(Xae,Ca,YZfp),(Xae,Ca,YZfq),(Xae,Ca,YZfr),(Xae,Ca,YZfs),(Xae,Ca,YZft),(Xae, Ca,YZfu),(Xae,Ca,YZfv),(Xae,Ca,YZfw),(Xae,Ca,YZfx),(Xae,Ca,YZfy),(Xae,Ca,YZfz), (Xae,Ca,YZga),(Xae,Ca,YZgb),(Xae,Ca,YZgc),(Xae,Ca,YZgd),(Xae,Ca,YZge),(Xae, Ca,YZgf),(Xae,Ca,YZgg),(Xae,Ca,YZgh),(Xae,Ca,YZgi),(Xae,Ca,YZgj),(Xae,Ca, YZgk),(Xae,Ca,YZgl),(Xae,Ca,YZgm),(Xae,Ca,YZgn),(Xae,Ca,YZgo),(Xae,Ca,YZgp), (Xae,Ca,YZgq),(Xae,Ca,YZgr),(Xae,Ca,YZgs),(Xae,Ca,YZgt),(Xae,Cb,YZa),(Xae,Cb, YZb),(Xae,Cb,YZc),(Xae,Cb,YZd),(Xae,Cb,YZe),(Xae,Cb,YZf),(Xae,Cb,YZg),(Xae, Cb,YZh),(Xae,Cb,YZi),(Xae,Cb,YZj),(Xae,Cb,YZk),(Xae,Cb,YZl),(Xae,Cb,YZm),(Xae, Cb,YZn),(Xae,Cb,YZo),(Xae,Cb,YZp),(Xae,Cb,YZq),(Xae,Cb,YZr),(Xae,Cb,YZs), (Xae,Cb,YZt),(Xae,Cb,YZu),(Xae,Cb,YZv),(Xae,Cb,YZw),(Xae,Cb,YZx),(Xae,Cb,YZy), (Xae,Cb,YZz),(Xae,Cb,YZaa),(Xae,Cb,YZab),(Xae,Cb,YZac),(Xae,Cb,YZad),(Xae, Cb,YZae),(Xae,Cb,YZaf),(Xae,Cb,YZag),(Xae,Cb,YZah),(Xae,Cb,YZai),(Xae,Cb,YZaj), (Xae,Cb,YZak),(Xae,Cb,YZal),(Xae,Cb,YZam),(Xae,Cb,YZan),(Xae,Cb,YZao),(Xae, Cb,YZap),(Xae,Cb,YZaq),(Xae,Cb,YZar),(Xae,Cb,YZas),(Xae,Cb,YZat),(Xae,Cb, YZau),(Xae,Cb,YZav),(Xae,Cb,YZaw),(Xae,Cb,YZax),(Xae,Cb,YZay),(Xae,Cb,YZaz), (Xae,Cb,YZba),(Xae,Cb,YZbb),(Xae,Cb,YZbc),(Xae,Cb,YZbd),(Xae,Cb,YZbe),(Xae, Cb,YZbf),(Xae,Cb,YZbg),(Xae,Cb,YZbh),(Xae,Cb,YZbi),(Xae,Cb,YZbj),(Xae,Cb,YZbk), (Xae,Cb,YZbl),(Xae,Cb,YZbm),(Xae,Cb,YZbn),(Xae,Cb,YZbo),(Xae,Cb,YZbp), (Xae,Cb,YZbq),(Xae,Cb,YZbr),(Xae,Cb,YZbs),(Xae,Cb,YZbt),(Xae,Cb,YZbu),(Xae,Cb, YZbv),(Xae,Cb,YZbw),(Xae,Cb,YZbx),(Xae,Cb,YZby),(Xae,Cb,YZbz),(Xae,Cb,YZca), (Xae,Cb,YZcb),(Xae,Cb,YZcc),(Xae,Cb,YZcd),(Xae,Cb,YZce),(Xae,Cb,YZcf),(Xae, Cb,YZcg),(Xae,Cb,YZch),(Xae,Cb,YZci),(Xae,Cb,YZcj),(Xae,Cb,YZck),(Xae,Cb, YZcl),(Xae,Cb,YZcm),(Xae,Cb,YZcn),(Xae,Cb,YZco),(Xae,Cb,YZcp),(Xae,Cb,YZcq), (Xae,Cb,YZcr),(Xae,Cb,YZcs),(Xae,Cb,YZct),(Xae,Cb,YZcu),(Xae,Cb,YZcv),(Xae, Cb,YZcw),(Xae,Cb,YZcx),(Xae,Cb,YZcy),(Xae,Cb,YZcz),(Xae,Cb,YZda),(Xae,Cb,YZdb), (Xae,Cb,YZdc),(Xae,Cb,YZdd),(Xae,Cb,YZde),(Xae,Cb,YZdf),(Xae,Cb,YZdg),(Xae, Cb,YZdh),(Xae,Cb,YZdi),(Xae,Cb,YZdj),(Xae,Cb,YZdk),(Xae,Cb,YZdl),(Xae,Cb, YZdm),(Xae,Cb,YZdn),(Xae,Cb,YZdo),(Xae,Cb,YZdp),(Xae,Cb,YZdq),(Xae,Cb,YZdr), (Xae,Cb,YZds),(Xae,Cb,Yzdt),(Xae,Cb,YZdu),(Xae,Cb,YZdv),(Xae,Cb,YZdw),(Xae, Cb,YZdx),(Xae,Cb,YZdy),(Xae,Cb,YZdz),(Xae,Cb,YZea),(Xae,Cb,YZeb),(Xae,Cb, YZec),(Xae,Cb,YZed),(Xae,Cb,YZee),(Xae,Cb,YZef),(Xae,Cb,YZeg),(Xae,Cb,YZeh), (Xae,Cb,YZei),(Xae,Cb,YZej),(Xae,Cb,YZek),(Xae,Cb,YZel),(Xae,Cb,YZem),(Xae,Cb, YZen),(Xae,Cb,YZeo),(Xae,Cb,YZep),(Xae,Cb,YZeq),(Xae,Cb,YZer),(Xae,Cb,YZes), (Xae,Cb,YZet),(Xae,Cb,YZeu),(Xae,Cb,YZev),(Xae,Cb,YZew),(Xae,Cb,YZex),(Xae, Cb,YZey),(Xae,Cb,YZez),(Xae,Cb,YZfa),(Xae,Cb,YZfb),(Xae,Cb,YZfc),(Xae,Cb,YZfd), (Xae,Cb,YZfe),(Xae,Cb,YZff),(Xae,Cb,YZfg),(Xae,Cb,YZfh),(Xae,Cb,YZfi),(Xae, Cb,YZfj),(Xae,Cb,YZfk),(Xae,Cb,YZfl),(Xae,Cb,YZfm),(Xae,Cb,YZfn),(Xae,Cb,YZfo), (Xae,Cb,YZfp),(Xae,Cb,YZfq),(Xae,Cb,YZfr),(Xae,Cb,YZfs),(Xae,Cb,YZft),(Xae, Cb,YZfu),(Xae,Cb,YZfv),(Xae,Cb,YZfw),(Xae,Cb,YZfx),(Xae,Cb,YZfy),(Xae,Cb,YZfz), (Xae,Cb,YZga),(Xae,Cb,YZgb),(Xae,Cb,YZgc),(Xae,Cb,YZgd),(Xae,Cb,YZge),(Xae, Cb,YZgf),(Xae,Cb,YZgg),(Xae,Cb,YZgh),(Xae,Cb,YZgi),(Xae,Cb,YZgj),(Xae,Cb, YZgk),(Xae,Cb,YZgl),(Xae,Cb,YZgm),(Xae,Cb,YZgn),(Xae,Cb,YZgo),(Xae,Cb,YZgp), (Xae,Cb,YZgq),(Xae,Cb,YZgr),(Xae,Cb,YZgs),(Xae,Cb,YZgt),(Xae,Cc,YZa),(Xae, Cc,YZb),(Xae,Cc,YZc),(Xae,Cc,YZd),(Xae,Cc,YZe),(Xae,Cc,YZf),(Xae,Cc,YZg),(Xae, Cc,YZh),(Xae,Cc,YZi),(Xae,Cc,YZj),(Xae,Cc,YZk),(Xae,Cc,YZl),(Xae,Cc,YZm), (Xae,Cc,YZn),(Xae,Cc,YZo),(Xae,Cc,YZp),(Xae,Cc,YZq),(Xae,Cc,YZr),(Xae,Cc,YZs), (Xae,Cc,YZt),(Xae,Cc,YZu),(Xae,Cc,YZv),(Xae,Cc,YZw),(Xae,Cc,YZx),(Xae,Cc, YZy),(Xae,Cc,YZz),(Xae,Cc,YZaa),(Xae,Cc,YZab),(Xae,Cc,YZac),(Xae,Cc,YZad),(Xae, Cc,YZae),(Xae,Cc,YZaf),(Xae,Cc,YZag),(Xae,Cc,YZah),(Xae,Cc,YZai),(Xae,Cc,YZaj), (Xae,Cc,YZak),(Xae,Cc,YZal),(Xae,Cc,YZam),(Xae,Cc,YZan),(Xae,Cc,YZao),(Xae, Cc,YZap),(Xae,Cc,YZaq),(Xae,Cc,YZar),(Xae,Cc,YZas),(Xae,Cc,YZat),(Xae,Cc, YZau),(Xae,Cc,YZav),(Xae,Cc,YZaw),(Xae,Cc,YZax),(Xae,Cc,YZay),(Xae,Cc,YZaz), (Xae,Cc,YZba),(Xae,Cc,YZbb),(Xae,Cc,YZbc),(Xae,Cc,YZbd),(Xae,Cc,YZbe),(Xae, Cc,YZbf),(Xae,Cc,YZbg),(Xae,Cc,YZbh),(Xae,Cc,YZbi),(Xae,Cc,YZbj),(Xae,Cc,YZbk), (Xae,Cc,YZbl),(Xae,Cc,YZbm),(Xae,Cc,YZbn),(Xae,Cc,YZbo),(Xae,Cc,YZbp),(Xae, Cc,YZbq),(Xae,Cc,YZbr),(Xae,Cc,YZbs),(Xae,Cc,YZbt),(Xae,Cc,YZbu),(Xae,Cc,YZbv), (Xae,Cc,YZbw),(Xae,Cc,YZbx),(Xae,Cc,YZby),(Xae,Cc,YZbz),(Xae,Cc,YZca),(Xae, Cc,YZcb),(Xae,Cc,YZcc),(Xae,Cc,YZcd),(Xae,Cc,YZce),(Xae,Cc,YZcf),(Xae,Cc, YZcg),(Xae,Cc,YZch),(Xae,Cc,YZci),(Xae,Cc,YZcj),(Xae,Cc,YZck),(Xae,Cc,YZcl),(Xae, Cc,YZcm),(Xae,Cc,YZcn),(Xae,Cc,YZco),(Xae,Cc,YZcp),(Xae,Cc,YZcq),(Xae,Cc, YZcr),(Xae,Cc,YZcs),(Xae,Cc,YZct),(Xae,Cc,YZcu),(Xae,Cc,YZcv),(Xae,Cc,YZcw), (Xae,Cc,YZcx),(Xae,Cc,YZcy),(Xae,Cc,YZcz),(Xae,Cc,YZda),(Xae,Cc,YZdb),(Xae, Cc,YZdc),(Xae,Cc,YZdd),(Xae,Cc,YZde),(Xae,Cc,YZdf),(Xae,Cc,YZdg),(Xae,Cc,YZdh), (Xae,Cc,YZdi),(Xae,Cc,YZdj),(Xae,Cc,YZdk),(Xae,Cc,YZdl),(Xae,Cc,YZdm),(Xae, Cc,YZdn),(Xae,Cc,YZdo),(Xae,Cc,YZdp),(Xae,Cc,YZdq),(Xae,Cc,YZdr),(Xae,Cc, YZds),(Xae,Cc,Yzdt),(Xae,Cc,YZdu),(Xae,Cc,YZdv),(Xae,Cc,YZdw),(Xae,Cc,YZdx), (Xae,Cc,YZdy),(Xae,Cc,YZdz),(Xae,Cc,YZea),(Xae,Cc,YZeb),(Xae,Cc,YZec),(Xae,Cc, YZed),(Xae,Cc,YZee),(Xae,Cc,YZef),(Xae,Cc,YZeg),(Xae,Cc,YZeh),(Xae,Cc,YZei), (Xae,Cc,YZej),(Xae,Cc,YZek),(Xae,Cc,YZel),(Xae,Cc,YZem),(Xae,Cc,YZen),(Xae, Cc,YZeo),(Xae,Cc,YZep),(Xae,Cc,YZeq),(Xae,Cc,YZer),(Xae,Cc,YZes),(Xae,Cc,YZet), (Xae,Cc,YZeu),(Xae,Cc,YZev),(Xae,Cc,YZew),(Xae,Cc,YZex),(Xae,Cc,YZey),(Xae, Cc,YZez),(Xae,Cc,YZfa),(Xae,Cc,YZfb),(Xae,Cc,YZfc),(Xae,Cc,YZfd),(Xae,Cc,YZfe), (Xae,Cc,YZff),(Xae,Cc,YZfg),(Xae,Cc,YZfh),(Xae,Cc,YZfi),(Xae,Cc,YZfj),(Xae, Cc,YZfk),(Xae,Cc,YZfl),(Xae,Cc,YZfm),(Xae,Cc,YZfn),(Xae,Cc,YZfo),(Xae,Cc,YZfp), (Xae,Cc,YZfq),(Xae,Cc,YZfr),(Xae,Cc,YZfs),(Xae,Cc,YZft),(Xae,Cc,YZfu),(Xae,Cc, YZfv),(Xae,Cc,YZfw),(Xae,Cc,YZfx),(Xae,Cc,YZfy),(Xae,Cc,YZfz),(Xae,Cc,YZga), (Xae,Cc,YZgb),(Xae,Cc,YZgc),(Xae,Cc,YZgd),(Xae,Cc,YZge),(Xae,Cc,YZgf),(Xae, Cc,YZgg),(Xae,Cc,YZgh),(Xae,Cc,YZgi),(Xae,Cc,YZgj),(Xae,Cc,YZgk),(Xae,Cc,YZgl), (Xae,Cc,YZgm),(Xae,Cc,YZgn),(Xae,Cc,YZgo),(Xae,Cc,YZgp),(Xae,Cc,YZgq),(Xae, Cc,YZgr),(Xae,Cc,YZgs),(Xae,Cc,YZgt),(Xae,Cd,YZa),(Xae,Cd,YZb),(Xae,Cd,YZc), (Xae,Cd,YZd),(Xae,Cd,YZe),(Xae,Cd,YZf),(Xae,Cd,YZg),(Xae,Cd,YZh),(Xae,Cd, YZi),(Xae,Cd,YZj),(Xae,Cd,YZk),(Xae,Cd,YZl),(Xae,Cd,YZm),(Xae,Cd,YZn),(Xae, Cd,YZo),(Xae,Cd,YZp),(Xae,Cd,YZq),(Xae,Cd,YZr),(Xae,Cd,YZs),(Xae,Cd,YZt),(Xae, Cd,YZu),(Xae,Cd,YZv),(Xae,Cd,YZw),(Xae,Cd,YZx),(Xae,Cd,YZy),(Xae,Cd,YZz), (Xae,Cd,YZaa),(Xae,Cd,YZab),(Xae,Cd,YZac),(Xae,Cd,YZad),(Xae,Cd,YZae),(Xae, Cd,YZaf),(Xae,Cd,YZag),(Xae,Cd,YZah),(Xae,Cd,YZai),(Xae,Cd,YZaj),(Xae,Cd,YZak), (Xae,Cd,YZal),(Xae,Cd,YZam),(Xae,Cd,YZan),(Xae,Cd,YZao),(Xae,Cd,YZap),(Xae, Cd,YZaq),(Xae,Cd,YZar),(Xae,Cd,YZas),(Xae,Cd,YZat),(Xae,Cd,YZau),(Xae,Cd,YZav), (Xae,Cd,YZaw),(Xae,Cd,YZax),(Xae,Cd,YZay),(Xae,Cd,YZaz),(Xae,Cd,YZba),(Xae, Cd,YZbb),(Xae,Cd,YZbc),(Xae,Cd,YZbd),(Xae,Cd,YZbe),(Xae,Cd,YZbf),(Xae,Cd, YZbg),(Xae,Cd,YZbh),(Xae,Cd,YZbi),(Xae,Cd,YZbj),(Xae,Cd,YZbk),(Xae,Cd,YZbl), (Xae,Cd,YZbm),(Xae,Cd,YZbn),(Xae,Cd,YZbo),(Xae,Cd,YZbp),(Xae,Cd,YZbq),(Xae, Cd,YZbr),(Xae,Cd,YZbs),(Xae,Cd,YZbt),(Xae,Cd,YZbu),(Xae,Cd,YZbv),(Xae,Cd, YZbw),(Xae,Cd,YZbx),(Xae,Cd,YZby),(Xae,Cd,YZbz),(Xae,Cd,YZca),(Xae,Cd,YZcb), (Xae,Cd,YZcc),(Xae,Cd,YZcd),(Xae,Cd,YZce),(Xae,Cd,YZcf),(Xae,Cd,YZcg),(Xae, Cd,YZch),(Xae,Cd,YZci),(Xae,Cd,YZcj),(Xae,Cd,YZck),(Xae,Cd,YZcl),(Xae,Cd,YZcm), (Xae,Cd,YZcn),(Xae,Cd,YZco),(Xae,Cd,YZcp),(Xae,Cd,YZcq),(Xae,Cd,YZcr),(Xae, Cd,YZcs),(Xae,Cd,YZct),(Xae,Cd,YZcu),(Xae,Cd,YZcv),(Xae,Cd,YZcw),(Xae,Cd, YZcx),(Xae,Cd,YZcy),(Xae,Cd,YZcz),(Xae,Cd,YZda),(Xae,Cd,YZdb),(Xae,Cd,YZdc), (Xae,Cd,YZdd),(Xae,Cd,YZde),(Xae,Cd,YZdf),(Xae,Cd,YZdg),(Xae,Cd,YZdh),(Xae, Cd,YZdi),(Xae,Cd,YZdj),(Xae,Cd,YZdk),(Xae,Cd,YZdl),(Xae,Cd,YZdm),(Xae,Cd,YZdn), (Xae,Cd,YZdo),(Xae,Cd,YZdp),(Xae,Cd,YZdq),(Xae,Cd,YZdr),(Xae,Cd,YZds),(Xae, Cd,Yzdt),(Xae,Cd,YZdu),(Xae,Cd,YZdv),(Xae,Cd,YZdw),(Xae,Cd,YZdx),(Xae,Cd, YZdy),(Xae,Cd,YZdz),(Xae,Cd,YZea),(Xae,Cd,YZeb),(Xae,Cd,YZec),(Xae,Cd,YZed), (Xae,Cd,YZee),(Xae,Cd,YZef),(Xae,Cd,YZeg),(Xae,Cd,YZeh),(Xae,Cd,YZei),(Xae, Cd,YZej),(Xae,Cd,YZek),(Xae,Cd,YZel),(Xae,Cd,YZem),(Xae,Cd,YZen),(Xae,Cd,YZeo), (Xae,Cd,YZep),(Xae,Cd,YZeq),(Xae,Cd,YZer),(Xae,Cd,YZes),(Xae,Cd,YZet),(Xae, Cd,YZeu),(Xae,Cd,YZev),(Xae,Cd,YZew),(Xae,Cd,YZex),(Xae,Cd,YZey),(Xae,Cd, YZez),(Xae,Cd,YZfa),(Xae,Cd,YZfb),(Xae,Cd,YZfc),(Xae,Cd,YZfd),(Xae,Cd,YZfe),(Xae, Cd,YZff),(Xae,Cd,YZfg),(Xae,Cd,YZfh),(Xae,Cd,YZfi),(Xae,Cd,YZfj),(Xae,Cd, YZfk),(Xae,Cd,YZfl),(Xae,Cd,YZfm),(Xae,Cd,YZfn),(Xae,Cd,YZfo),(Xae,Cd,YZfp),(Xae, Cd,YZfq),(Xae,Cd,YZfr),(Xae,Cd,YZfs),(Xae,Cd,YZft),(Xae,Cd,YZfu),(Xae,Cd, YZfv),(Xae,Cd,YZfw),(Xae,Cd,YZfx),(Xae,Cd,YZfy),(Xae,Cd,YZfz),(Xae,Cd,YZga), (Xae,Cd,YZgb),(Xae,Cd,YZgc),(Xae,Cd,YZgd),(Xae,Cd,YZge),(Xae,Cd,YZgf),(Xae, Cd,YZgg),(Xae,Cd,YZgh),(Xae,Cd,YZgi),(Xae,Cd,YZgj),(Xae,Cd,YZgk),(Xae,Cd,YZgl), (Xae,Cd,YZgm),(Xae,Cd,YZgn),(Xae,Cd,YZgo),(Xae,Cd,YZgp),(Xae,Cd,YZgq),(Xae, Cd,YZgr),(Xae,Cd,YZgs),(Xae,Cd,YZgt),(Xae,Ce,YZa),(Xae,Ce,YZb),(Xae,Ce,YZc), (Xae,Ce,YZd),(Xae,Ce,YZe),(Xae,Ce,YZf),(Xae,Ce,YZg),(Xae,Ce,YZh),(Xae,Ce, YZi),(Xae,Ce,YZj),(Xae,Ce,YZk),(Xae,Ce,YZl),(Xae,Ce,YZm),(Xae,Ce,YZn),(Xae,Ce, YZo),(Xae,Ce,YZp),(Xae,Ce,YZq),(Xae,Ce,YZr),(Xae,Ce,YZs),(Xae,Ce,YZt),(Xae, Ce,YZu),(Xae,Ce,YZv),(Xae,Ce,YZw),(Xae,Ce,YZx),(Xae,Ce,YZy),(Xae,Ce,YZz),(Xae, Ce,YZaa),(Xae,Ce,YZab),(Xae,Ce,YZac),(Xae,Ce,YZad),(Xae,Ce,YZae),(Xae,Ce, YZaf),(Xae,Ce,YZag),(Xae,Ce,YZah),(Xae,Ce,YZai),(Xae,Ce,YZaj),(Xae,Ce,YZak),(Xae, Ce,YZal),(Xae,Ce,YZam),(Xae,Ce,YZan),(Xae,Ce,YZao),(Xae,Ce,YZap),(Xae,Ce, YZaq),(Xae,Ce,YZar),(Xae,Ce,YZas),(Xae,Ce,YZat),(Xae,Ce,YZau),(Xae,Ce,YZav), (Xae,Ce,YZaw),(Xae,Ce,YZax),(Xae,Ce,YZay),(Xae,Ce,YZaz),(Xae,Ce,YZba),(Xae, Ce,YZbb),(Xae,Ce,YZbc),(Xae,Ce,YZbd),(Xae,Ce,YZbe),(Xae,Ce,YZbf),(Xae,Ce,YZbg), (Xae,Ce,YZbh),(Xae,Ce,YZbi),(Xae,Ce,YZbj),(Xae,Ce,YZbk),(Xae,Ce,YZbl),(Xae, Ce,YZbm),(Xae,Ce,YZbn),(Xae,Ce,YZbo),(Xae,Ce,YZbp),(Xae,Ce,YZbq),(Xae,Ce, YZbr),(Xae,Ce,YZbs),(Xae,Ce,YZbt),(Xae,Ce,YZbu),(Xae,Ce,YZbv),(Xae,Ce,YZbw), (Xae,Ce,YZbx),(Xae,Ce,YZby),(Xae,Ce,YZbz),(Xae,Ce,YZca),(Xae,Ce,YZcb),(Xae,Ce, YZcc),(Xae,Ce,YZcd),(Xae,Ce,YZce),(Xae,Ce,YZcf),(Xae,Ce,YZcg),(Xae,Ce,YZch), (Xae,Ce,YZci),(Xae,Ce,YZcj),(Xae,Ce,YZck),(Xae,Ce,YZcl),(Xae,Ce,YZcm),(Xae, Ce,YZcn),(Xae,Ce,YZco),(Xae,Ce,YZcp),(Xae,Ce,YZcq),(Xae,Ce,YZcr),(Xae,Ce,YZcs), (Xae,Ce,YZct),(Xae,Ce,YZcu),(Xae,Ce,YZcv),(Xae,Ce,YZcw),(Xae,Ce,YZcx),(Xae, Ce,YZcy),(Xae,Ce,YZcz),(Xae,Ce,YZda),(Xae,Ce,YZdb),(Xae,Ce,YZdc),(Xae,Ce,YZdd), (Xae,Ce,YZde),(Xae,Ce,YZdf),(Xae,Ce,YZdg),(Xae,Ce,YZdh),(Xae,Ce,YZdi),(Xae, Ce,YZdj),(Xae,Ce,YZdk),(Xae,Ce,YZdl),(Xae,Ce,YZdm),(Xae,Ce,YZdn),(Xae,Ce, YZdo),(Xae,Ce,YZdp),(Xae,Ce,YZdq),(Xae,Ce,YZdr),(Xae,Ce,YZds),(Xae,Ce,Yzdt), (Xae,Ce,YZdu),(Xae,Ce,YZdv),(Xae,Ce,YZdw),(Xae,Ce,YZdx),(Xae,Ce,YZdy),(Xae, Ce,YZdz),(Xae,Ce,YZea),(Xae,Ce,YZeb),(Xae,Ce,YZec),(Xae,Ce,YZed),(Xae,Ce,YZee), (Xae,Ce,YZef),(Xae,Ce,YZeg),(Xae,Ce,YZeh),(Xae,Ce,YZei),(Xae,Ce,YZej),(Xae, Ce,YZek),(Xae,Ce,YZel),(Xae,Ce,YZem),(Xae,Ce,YZen),(Xae,Ce,YZeo),(Xae,Ce, YZep),(Xae,Ce,YZeq),(Xae,Ce,YZer),(Xae,Ce,YZes),(Xae,Ce,YZet),(Xae,Ce,YZeu),(Xae, Ce,YZev),(Xae,Ce,YZew),(Xae,Ce,YZex),(Xae,Ce,YZey),(Xae,Ce,YZez),(Xae,Ce, YZfa),(Xae,Ce,YZfb),(Xae,Ce,YZfc),(Xae,Ce,YZfd),(Xae,Ce,YZfe),(Xae,Ce,YZff),(Xae, Ce,YZfg),(Xae,Ce,YZfh),(Xae,Ce,YZfi),(Xae,Ce,YZfj),(Xae,Ce,YZfk),(Xae,Ce, YZfl),(Xae,Ce,YZfm),(Xae,Ce,YZfn),(Xae,Ce,YZfo),(Xae,Ce,YZfp),(Xae,Ce,YZfq),(Xae, Ce,YZfr),(Xae,Ce,YZfs),(Xae,Ce,YZft),(Xae,Ce,YZfu),(Xae,Ce,YZfv),(Xae,Ce,YZfw), (Xae,Ce,YZfx),(Xae,Ce,YZfy),(Xae,Ce,YZfz),(Xae,Ce,YZga),(Xae,Ce,YZgb),(Xae, Ce,YZgc),(Xae,Ce,YZgd),(Xae,Ce,YZge),(Xae,Ce,YZgf),(Xae,Ce,YZgg),(Xae,Ce, YZgh),(Xae,Ce,YZgi),(Xae,Ce,YZgj),(Xae,Ce,YZgk),(Xae,Ce,YZgl),(Xae,Ce,YZgm), (Xae,Ce,YZgn),(Xae,Ce,YZgo),(Xae,Ce,YZgp),(Xae,Ce,YZgq),(Xae,Ce,YZgr),(Xae, Ce,YZgs),(Xae,Ce,YZgt),(Xae,Cf,YZa),(Xae,Cf,YZb),(Xae,Cf,YZc),(Xae,Cf,YZd),(Xae, Cf,YZe),(Xae,Cf,YZf),(Xae,Cf,YZg),(Xae,Cf,YZh),(Xae,Cf,YZi),(Xae,Cf,YZj),(Xae, Cf,YZk),(Xae,Cf,YZl),(Xae,Cf,YZm),(Xae,Cf,YZn),(Xae,Cf,YZo),(Xae,Cf,YZp),(Xae, Cf,YZq),(Xae,Cf,YZr),(Xae,Cf,YZs),(Xae,Cf,YZt),(Xae,Cf,YZu),(Xae,Cf,YZv),(Xae, Cf,YZw),(Xae,Cf,YZx),(Xae,Cf,YZy),(Xae,Cf,YZz),(Xae,Cf,YZaa),(Xae,Cf,YZab), (Xae,Cf,YZac),(Xae,Cf,YZad),(Xae,Cf,YZae),(Xae,Cf,YZaf),(Xae,Cf,YZag),(Xae,Cf, YZah),(Xae,Cf,YZai),(Xae,Cf,YZaj),(Xae,Cf,YZak),(Xae,Cf,YZal),(Xae,Cf,YZam),(Xae, Cf,YZan),(Xae,Cf,YZao),(Xae,Cf,YZap),(Xae,Cf,YZaq),(Xae,Cf,YZar),(Xae,Cf, YZas),(Xae,Cf,YZat),(Xae,Cf,YZau),(Xae,Cf,YZav),(Xae,Cf,YZaw),(Xae,Cf,YZax),(Xae, Cf,YZay),(Xae,Cf,YZaz),(Xae,Cf,YZba),(Xae,Cf,YZbb),(Xae,Cf,YZbc),(Xae,Cf, YZbd),(Xae,Cf,YZbe),(Xae,Cf,YZbf),(Xae,Cf,YZbg),(Xae,Cf,YZbh),(Xae,Cf,YZbi),(Xae, Cf,YZbj),(Xae,Cf,YZbk),(Xae,Cf,YZbl),(Xae,Cf,YZbm),(Xae,Cf,YZbn),(Xae,Cf, YZbo),(Xae,Cf,YZbp),(Xae,Cf,YZbq),(Xae,Cf,YZbr),(Xae,Cf,YZbs),(Xae,Cf,YZbt),(Xae, Cf,YZbu),(Xae,Cf,YZbv),(Xae,Cf,YZbw),(Xae,Cf,YZbx),(Xae,Cf,YZby),(Xae,Cf, YZbz),(Xae,Cf,YZca),(Xae,Cf,YZcb),(Xae,Cf,YZcc),(Xae,Cf,YZcd),(Xae,Cf,YZce),(Xae, Cf,YZcf),(Xae,Cf,YZcg),(Xae,Cf,YZch),(Xae,Cf,YZci),(Xae,Cf,YZcj),(Xae,Cf,YZck), (Xae,Cf,YZcl),(Xae,Cf,YZcm),(Xae,Cf,YZcn),(Xae,Cf,YZco),(Xae,Cf,YZcp),(Xae, Cf,YZcq),(Xae,Cf,YZcr),(Xae,Cf,YZcs),(Xae,Cf,YZct),(Xae,Cf,YZcu),(Xae,Cf,YZcv), (Xae,Cf,YZcw),(Xae,Cf,YZcx),(Xae,Cf,YZcy),(Xae,Cf,YZcz),(Xae,Cf,YZda),(Xae, Cf,YZdb),(Xae,Cf,YZdc),(Xae,Cf,YZdd),(Xae,Cf,YZde),(Xae,Cf,YZdf),(Xae,Cf,YZdg), (Xae,Cf,YZdh),(Xae,Cf,YZdi),(Xae,Cf,YZdj),(Xae,Cf,YZdk),(Xae,Cf,YZdl),(Xae, Cf,YZdm),(Xae,Cf,YZdn),(Xae,Cf,YZdo),(Xae,Cf,YZdp),(Xae,Cf,YZdq),(Xae,Cf,YZdr), (Xae,Cf,YZds),(Xae,Cf,Yzdt),(Xae,Cf,YZdu),(Xae,Cf,YZdv),(Xae,Cf,YZdw),(Xae, Cf,YZdx),(Xae,Cf,YZdy),(Xae,Cf,YZdz),(Xae,Cf,YZea),(Xae,Cf,YZeb),(Xae,Cf,YZec), (Xae,Cf,YZed),(Xae,Cf,YZee),(Xae,Cf,YZef),(Xae,Cf,YZeg),(Xae,Cf,YZeh),(Xae, Cf,YZei),(Xae,Cf,YZej),(Xae,Cf,YZek),(Xae,Cf,YZel),(Xae,Cf,YZem),(Xae,Cf,YZen), (Xae,Cf,YZeo),(Xae,Cf,YZep),(Xae,Cf,YZeq),(Xae,Cf,YZer),(Xae,Cf,YZes),(Xae,Cf, YZet),(Xae,Cf,YZeu),(Xae,Cf,YZev),(Xae,Cf,YZew),(Xae,Cf,YZex),(Xae,Cf,YZey),(Xae, Cf,YZez),(Xae,Cf,YZfa),(Xae,Cf,YZfb),(Xae,Cf,YZfc),(Xae,Cf,YZfd),(Xae,Cf,YZfe), (Xae,Cf,YZff),(Xae,Cf,YZfg),(Xae,Cf,YZfh),(Xae,Cf,YZfi),(Xae,Cf,YZfj),(Xae, Cf,YZfk),(Xae,Cf,YZfl),(Xae,Cf,YZfm),(Xae,Cf,YZfn),(Xae,Cf,YZfo),(Xae,Cf,YZfp), (Xae,Cf,YZfq),(Xae,Cf,YZfr),(Xae,Cf,YZfs),(Xae,Cf,YZft),(Xae,Cf,YZfu),(Xae,Cf,YZfv), (Xae,Cf,YZfw),(Xae,Cf,YZfx),(Xae,Cf,YZfy),(Xae,Cf,YZfz),(Xae,Cf,YZga),(Xae, Cf,YZgb),(Xae,Cf,YZgc),(Xae,Cf,YZgd),(Xae,Cf,YZge),(Xae,Cf,YZgf),(Xae,Cf,YZgg), (Xae,Cf,YZgh),(Xae,Cf,YZgi),(Xae,Cf,YZgj),(Xae,Cf,YZgk),(Xae,Cf,YZgl),(Xae, Cf,YZgm),(Xae,Cf,YZgn),(Xae,Cf,YZgo),(Xae,Cf,YZgp),(Xae,Cf,YZgq),(Xae,Cf,YZgr), (Xae,Cf,YZgs),(Xae,Cf,YZgt), (Xaf,Ca,YZa),(Xaf,Ca,YZb),(Xaf,Ca,YZc),(Xaf,Ca,YZd),(Xaf,Ca,YZe),(Xaf,Ca,YZf), (Xaf,Ca,YZg),(Xaf,Ca,YZh),(Xaf,Ca,YZi),(Xaf,Ca,YZj),(Xaf,Ca,YZk),(Xaf,Ca,YZl),(Xaf, Ca,YZm),(Xaf,Ca,YZn),(Xaf,Ca,YZo),(Xaf,Ca,YZp),(Xaf,Ca,YZq),(Xaf,Ca,YZr), (Xaf,Ca,YZs),(Xaf,Ca,YZt),(Xaf,Ca,YZu),(Xaf,Ca,YZv),(Xaf,Ca,YZw),(Xaf,Ca,YZx), (Xaf,Ca,YZy),(Xaf,Ca,YZz),(Xaf,Ca,YZaa),(Xaf,Ca,YZab),(Xaf,Ca,YZac),(Xaf,Ca,YZad), (Xaf,Ca,YZae),(Xaf,Ca,YZaf),(Xaf,Ca,YZag),(Xaf,Ca,YZah),(Xaf,Ca,YZai),(Xaf, Ca,YZaj),(Xaf,Ca,YZak),(Xaf,Ca,YZal),(Xaf,Ca,YZam),(Xaf,Ca,YZan),(Xaf,Ca,YZao), (Xaf,Ca,YZap),(Xaf,Ca,YZaq),(Xaf,Ca,YZar),(Xaf,Ca,YZas),(Xaf,Ca,YZat),(Xaf, Ca,YZau),(Xaf,Ca,YZav),(Xaf,Ca,YZaw),(Xaf,Ca,YZax),(Xaf,Ca,YZay),(Xaf,Ca,YZaz), (Xaf,Ca,YZba),(Xaf,Ca,YZbb),(Xaf,Ca,YZbc),(Xaf,Ca,YZbd),(Xaf,Ca,YZbe),(Xaf, Ca,YZbf),(Xaf,Ca,YZbg),(Xaf,Ca,YZbh),(Xaf,Ca,YZbi),(Xaf,Ca,YZbj),(Xaf,Ca,YZbk), (Xaf,Ca,YZbl),(Xaf,Ca,YZbm),(Xaf,Ca,YZbn),(Xaf,Ca,YZbo),(Xaf,Ca,YZbp),(Xaf, Ca,YZbq),(Xaf,Ca,YZbr),(Xaf,Ca,YZbs),(Xaf,Ca,YZbt),(Xaf,Ca,YZbu),(Xaf,Ca,YZbv), (Xaf,Ca,YZbw),(Xaf,Ca,YZbx),(Xaf,Ca,YZby),(Xaf,Ca,YZbz),(Xaf,Ca,YZca),(Xaf, Ca,YZcb),(Xaf,Ca,YZcc),(Xaf,Ca,YZcd),(Xaf,Ca,YZce),(Xaf,Ca,YZcf),(Xaf,Ca,YZcg), (Xaf,Ca,YZch),(Xaf,Ca,YZci),(Xaf,Ca,YZcj),(Xaf,Ca,YZck),(Xaf,Ca,YZcl),(Xaf,Ca, YZcm),(Xaf,Ca,YZcn),(Xaf,Ca,YZco),(Xaf,Ca,YZcp),(Xaf,Ca,YZcq),(Xaf,Ca,YZcr),(Xaf, Ca,YZcs),(Xaf,Ca,YZct),(Xaf,Ca,YZcu),(Xaf,Ca,YZcv),(Xaf,Ca,YZcw),(Xaf,Ca, YZcx),(Xaf,Ca,YZcy),(Xaf,Ca,YZcz),(Xaf,Ca,YZda),(Xaf,Ca,YZdb),(Xaf,Ca,YZdc),(Xaf, Ca,YZdd),(Xaf,Ca,YZde),(Xaf,Ca,YZdf),(Xaf,Ca,YZdg),(Xaf,Ca,YZdh),(Xaf,Ca, YZdi),(Xaf,Ca,YZdj),(Xaf,Ca,YZdk),(Xaf,Ca,YZdl),(Xaf,Ca,YZdm),(Xaf,Ca,YZdn),(Xaf, Ca,YZdo),(Xaf,Ca,YZdp),(Xaf,Ca,YZdq),(Xaf,Ca,YZdr),(Xaf,Ca,YZds),(Xaf,Ca, Yzdt),(Xaf,Ca,YZdu),(Xaf,Ca,YZdv),(Xaf,Ca,YZdw),(Xaf,Ca,YZdx),(Xaf,Ca,YZdy),(Xaf, Ca,YZdz),(Xaf,Ca,YZea),(Xaf,Ca,YZeb),(Xaf,Ca,YZec),(Xaf,Ca,YZed),(Xaf,Ca, YZee),(Xaf,Ca,YZef),(Xaf,Ca,YZeg),(Xaf,Ca,YZeh),(Xaf,Ca,YZei),(Xaf,Ca,YZej),(Xaf, Ca,YZek),(Xaf,Ca,YZel),(Xaf,Ca,YZem),(Xaf,Ca,YZen),(Xaf,Ca,YZeo),(Xaf,Ca,YZep), (Xaf,Ca,YZeq),(Xaf,Ca,YZer),(Xaf,Ca,YZes),(Xaf,Ca,YZet),(Xaf,Ca,YZeu),(Xaf, Ca,YZev),(Xaf,Ca,YZew),(Xaf,Ca,YZex),(Xaf,Ca,YZey),(Xaf,Ca,YZez),(Xaf,Ca,YZfa), (Xaf,Ca,YZfb),(Xaf,Ca,YZfc),(Xaf,Ca,YZfd),(Xaf,Ca,YZfe),(Xaf,Ca,YZff),(Xaf,Ca, YZfg),(Xaf,Ca,YZfh),(Xaf,Ca,YZfi),(Xaf,Ca,YZfj),(Xaf,Ca,YZfk),(Xaf,Ca,YZfl),(Xaf, Ca,YZfm),(Xaf,Ca,YZfn),(Xaf,Ca,YZfo),(Xaf,Ca,YZfp),(Xaf,Ca,YZfq),(Xaf,Ca,YZfr), (Xaf,Ca,YZfs),(Xaf,Ca,YZft),(Xaf,Ca,YZfu),(Xaf,Ca,YZfv),(Xaf,Ca,YZfw),(Xaf,Ca, YZfx),(Xaf,Ca,YZfy),(Xaf,Ca,YZfz),(Xaf,Ca,YZga),(Xaf,Ca,YZgb),(Xaf,Ca,YZgc),(Xaf, Ca,YZgd),(Xaf,Ca,YZge),(Xaf,Ca,YZgf),(Xaf,Ca,YZgg),(Xaf,Ca,YZgh),(Xaf,Ca, YZgi),(Xaf,Ca,YZgj),(Xaf,Ca,YZgk),(Xaf,Ca,YZgl),(Xaf,Ca,YZgm),(Xaf,Ca,YZgn),(Xaf, Ca,YZgo),(Xaf,Ca,YZgp),(Xaf,Ca,YZgq),(Xaf,Ca,YZgr),(Xaf,Ca,YZgs),(Xaf,Ca, YZgt),(Xaf,Cb,YZa),(Xaf,Cb,YZb),(Xaf,Cb,YZc),(Xaf,Cb,YZd),(Xaf,Cb,YZe),(Xaf,Cb, YZf),(Xaf,Cb,YZg),(Xaf,Cb,YZh),(Xaf,Cb,YZi),(Xaf,Cb,YZj),(Xaf,Cb,YZk),(Xaf,Cb, YZl),(Xaf,Cb,YZm),(Xaf,Cb,YZn),(Xaf,Cb,YZo),(Xaf,Cb,YZp),(Xaf,Cb,YZq),(Xaf, Cb,YZr),(Xaf,Cb,YZs),(Xaf,Cb,YZt),(Xaf,Cb,YZu),(Xaf,Cb,YZv),(Xaf,Cb,YZw),(Xaf, Cb,YZx),(Xaf,Cb,YZy),(Xaf,Cb,YZz),(Xaf,Cb,YZaa),(Xaf,Cb,YZab),(Xaf,Cb,YZac), (Xaf,Cb,YZad),(Xaf,Cb,YZae),(Xaf,Cb,YZaf),(Xaf,Cb,YZag),(Xaf,Cb,YZah),(Xaf,Cb, YZai),(Xaf,Cb,YZaj),(Xaf,Cb,YZak),(Xaf,Cb,YZal),(Xaf,Cb,YZam),(Xaf,Cb,YZan),(Xaf, Cb,YZao),(Xaf,Cb,YZap),(Xaf,Cb,YZaq),(Xaf,Cb,YZar),(Xaf,Cb,YZas),(Xaf,Cb, YZat),(Xaf,Cb,YZau),(Xaf,Cb,YZav),(Xaf,Cb,YZaw),(Xaf,Cb,YZax),(Xaf,Cb,YZay),(Xaf, Cb,YZaz),(Xaf,Cb,YZba),(Xaf,Cb,YZbb),(Xaf,Cb,YZbc),(Xaf,Cb,YZbd),(Xaf,Cb, YZbe),(Xaf,Cb,YZbf),(Xaf,Cb,YZbg),(Xaf,Cb,YZbh),(Xaf,Cb,YZbi),(Xaf,Cb,YZbj),(Xaf, Cb,YZbk),(Xaf,Cb,YZbl),(Xaf,Cb,YZbm),(Xaf,Cb,YZbn),(Xaf,Cb,YZbo),(Xaf,Cb, YZbp),(Xaf,Cb,YZbq),(Xaf,Cb,YZbr),(Xaf,Cb,YZbs),(Xaf,Cb,YZbt),(Xaf,Cb,YZbu), (Xaf,Cb,YZbv),(Xaf,Cb,YZbw),(Xaf,Cb,YZbx),(Xaf,Cb,YZby),(Xaf,Cb,YZbz),(Xaf, Cb,YZca),(Xaf,Cb,YZcb),(Xaf,Cb,YZcc),(Xaf,Cb,YZcd),(Xaf,Cb,YZce),(Xaf,Cb,YZcf), (Xaf,Cb,YZcg),(Xaf,Cb,YZch),(Xaf,Cb,YZci),(Xaf,Cb,YZcj),(Xaf,Cb,YZck),(Xaf, Cb,YZcl),(Xaf,Cb,YZcm),(Xaf,Cb,YZcn),(Xaf,Cb,YZco),(Xaf,Cb,YZcp),(Xaf,Cb,YZcq), (Xaf,Cb,YZcr),(Xaf,Cb,YZcs),(Xaf,Cb,YZct),(Xaf,Cb,YZcu),(Xaf,Cb,YZcv),(Xaf, Cb,YZcw),(Xaf,Cb,YZcx),(Xaf,Cb,YZcy),(Xaf,Cb,YZcz),(Xaf,Cb,YZda),(Xaf,Cb,YZdb), (Xaf,Cb,YZdc),(Xaf,Cb,YZdd),(Xaf,Cb,YZde),(Xaf,Cb,YZdf),(Xaf,Cb,YZdg),(Xaf, Cb,YZdh),(Xaf,Cb,YZdi),(Xaf,Cb,YZdj),(Xaf,Cb,YZdk),(Xaf,Cb,YZdl),(Xaf,Cb,YZdm), (Xaf,Cb,YZdn),(Xaf,Cb,YZdo),(Xaf,Cb,YZdp),(Xaf,Cb,YZdq),(Xaf,Cb,YZdr),(Xaf, Cb,YZds),(Xaf,Cb,Yzdt),(Xaf,Cb,YZdu),(Xaf,Cb,YZdv),(Xaf,Cb,YZdw),(Xaf,Cb,YZdx), (Xaf,Cb,YZdy),(Xaf,Cb,YZdz),(Xaf,Cb,YZea),(Xaf,Cb,YZeb),(Xaf,Cb,YZec),(Xaf, Cb,YZed),(Xaf,Cb,YZee),(Xaf,Cb,YZef),(Xaf,Cb,YZeg),(Xaf,Cb,YZeh),(Xaf,Cb,YZei), (Xaf,Cb,YZej),(Xaf,Cb,YZek),(Xaf,Cb,YZel),(Xaf,Cb,YZem),(Xaf,Cb,YZen),(Xaf, Cb,YZeo),(Xaf,Cb,YZep),(Xaf,Cb,YZeq),(Xaf,Cb,YZer),(Xaf,Cb,YZes),(Xaf,Cb,YZet), (Xaf,Cb,YZeu),(Xaf,Cb,YZev),(Xaf,Cb,YZew),(Xaf,Cb,YZex),(Xaf,Cb,YZey),(Xaf, Cb,YZez),(Xaf,Cb,YZfa),(Xaf,Cb,YZfb),(Xaf,Cb,YZfc),(Xaf,Cb,YZfd),(Xaf,Cb,YZfe), (Xaf,Cb,YZff),(Xaf,Cb,YZfg),(Xaf,Cb,YZfh),(Xaf,Cb,YZfi),(Xaf,Cb,YZfj),(Xaf,Cb, YZfk),(Xaf,Cb,YZfl),(Xaf,Cb,YZfm),(Xaf,Cb,YZfn),(Xaf,Cb,YZfo),(Xaf,Cb,YZfp), (Xaf,Cb,YZfq),(Xaf,Cb,YZfr),(Xaf,Cb,YZfs),(Xaf,Cb,YZft),(Xaf,Cb,YZfu),(Xaf,Cb,YZfv), (Xaf,Cb,YZfw),(Xaf,Cb,YZfx),(Xaf,Cb,YZfy),(Xaf,Cb,YZfz),(Xaf,Cb,YZga),(Xaf, Cb,YZgb),(Xaf,Cb,YZgc),(Xaf,Cb,YZgd),(Xaf,Cb,YZge),(Xaf,Cb,YZgf),(Xaf,Cb,YZgg), (Xaf,Cb,YZgh),(Xaf,Cb,YZgi),(Xaf,Cb,YZgj),(Xaf,Cb,YZgk),(Xaf,Cb,YZgl),(Xaf, Cb,YZgm),(Xaf,Cb,YZgn),(Xaf,Cb,YZgo),(Xaf,Cb,YZgp),(Xaf,Cb,YZgq),(Xaf,Cb, YZgr),(Xaf,Cb,YZgs),(Xaf,Cb,YZgt),(Xaf,Cc,YZa),(Xaf,Cc,YZb),(Xaf,Cc,YZc),(Xaf, Cc,YZd),(Xaf,Cc,YZe),(Xaf,Cc,YZf),(Xaf,Cc,YZg),(Xaf,Cc,YZh),(Xaf,Cc,YZi),(Xaf, Cc,YZj),(Xaf,Cc,YZk),(Xaf,Cc,YZl),(Xaf,Cc,YZm),(Xaf,Cc,YZn),(Xaf,Cc,YZo),(Xaf, Cc,YZp),(Xaf,Cc,YZq),(Xaf,Cc,YZr),(Xaf,Cc,YZs),(Xaf,Cc,YZt),(Xaf,Cc,YZu),(Xaf, Cc,YZv),(Xaf,Cc,YZw),(Xaf,Cc,YZx),(Xaf,Cc,YZy),(Xaf,Cc,YZz),(Xaf,Cc,YZaa),(Xaf, Cc,YZab),(Xaf,Cc,YZac),(Xaf,Cc,YZad),(Xaf,Cc,YZae),(Xaf,Cc,YZaf),(Xaf,Cc,YZag), (Xaf,Cc,YZah),(Xaf,Cc,YZai),(Xaf,Cc,YZaj),(Xaf,Cc,YZak),(Xaf,Cc,YZal),(Xaf, Cc,YZam),(Xaf,Cc,YZan),(Xaf,Cc,YZao),(Xaf,Cc,YZap),(Xaf,Cc,YZaq),(Xaf,Cc,YZar), (Xaf,Cc,YZas),(Xaf,Cc,YZat),(Xaf,Cc,YZau),(Xaf,Cc,YZav),(Xaf,Cc,YZaw),(Xaf, Cc,YZax),(Xaf,Cc,YZay),(Xaf,Cc,YZaz),(Xaf,Cc,YZba),(Xaf,Cc,YZbb),(Xaf,Cc,YZbc), (Xaf,Cc,YZbd),(Xaf,Cc,YZbe),(Xaf,Cc,YZbf),(Xaf,Cc,YZbg),(Xaf,Cc,YZbh),(Xaf, Cc,YZbi),(Xaf,Cc,YZbj),(Xaf,Cc,YZbk),(Xaf,Cc,YZbl),(Xaf,Cc,YZbm),(Xaf,Cc,YZbn), (Xaf,Cc,YZbo),(Xaf,Cc,YZbp),(Xaf,Cc,YZbq),(Xaf,Cc,YZbr),(Xaf,Cc,YZbs),(Xaf, Cc,YZbt),(Xaf,Cc,YZbu),(Xaf,Cc,YZbv),(Xaf,Cc,YZbw),(Xaf,Cc,YZbx),(Xaf,Cc,YZby), (Xaf,Cc,YZbz),(Xaf,Cc,YZca),(Xaf,Cc,YZcb),(Xaf,Cc,YZcc),(Xaf,Cc,YZcd),(Xaf, Cc,YZce),(Xaf,Cc,YZcf),(Xaf,Cc,YZcg),(Xaf,Cc,YZch),(Xaf,Cc,YZci),(Xaf,Cc,YZcj), (Xaf,Cc,YZck),(Xaf,Cc,YZcl),(Xaf,Cc,YZcm),(Xaf,Cc,YZcn),(Xaf,Cc,YZco),(Xaf,Cc, YZcp),(Xaf,Cc,YZcq),(Xaf,Cc,YZcr),(Xaf,Cc,YZcs),(Xaf,Cc,YZct),(Xaf,Cc,YZcu),(Xaf, Cc,YZcv),(Xaf,Cc,YZcw),(Xaf,Cc,YZcx),(Xaf,Cc,YZcy),(Xaf,Cc,YZcz),(Xaf,Cc, YZda),(Xaf,Cc,YZdb),(Xaf,Cc,YZdc),(Xaf,Cc,YZdd),(Xaf,Cc,YZde),(Xaf,Cc,YZdf),(Xaf, Cc,YZdg),(Xaf,Cc,YZdh),(Xaf,Cc,YZdi),(Xaf,Cc,YZdj),(Xaf,Cc,YZdk),(Xaf,Cc, YZdl),(Xaf,Cc,YZdm),(Xaf,Cc,YZdn),(Xaf,Cc,YZdo),(Xaf,Cc,YZdp),(Xaf,Cc,YZdq), (Xaf,Cc,YZdr),(Xaf,Cc,YZds),(Xaf,Cc,Yzdt),(Xaf,Cc,YZdu),(Xaf,Cc,YZdv),(Xaf,Cc, YZdw),(Xaf,Cc,YZdx),(Xaf,Cc,YZdy),(Xaf,Cc,YZdz),(Xaf,Cc,YZea),(Xaf,Cc,YZeb), (Xaf,Cc,YZec),(Xaf,Cc,YZed),(Xaf,Cc,YZee),(Xaf,Cc,YZef),(Xaf,Cc,YZeg),(Xaf,Cc, YZeh),(Xaf,Cc,YZei),(Xaf,Cc,YZej),(Xaf,Cc,YZek),(Xaf,Cc,YZel),(Xaf,Cc,YZem),(Xaf, Cc,YZen),(Xaf,Cc,YZeo),(Xaf,Cc,YZep),(Xaf,Cc,YZeq),(Xaf,Cc,YZer),(Xaf,Cc, YZes),(Xaf,Cc,YZet),(Xaf,Cc,YZeu),(Xaf,Cc,YZev),(Xaf,Cc,YZew),(Xaf,Cc,YZex),(Xaf, Cc,YZey),(Xaf,Cc,YZez),(Xaf,Cc,YZfa),(Xaf,Cc,YZfb),(Xaf,Cc,YZfc),(Xaf,Cc,YZfd), (Xaf,Cc,YZfe),(Xaf,Cc,YZff),(Xaf,Cc,YZfg),(Xaf,Cc,YZfh),(Xaf,Cc,YZfi),(Xaf, Cc,YZfj),(Xaf,Cc,YZfk),(Xaf,Cc,YZfl),(Xaf,Cc,YZfm),(Xaf,Cc,YZfn),(Xaf,Cc,YZfo), (Xaf,Cc,YZfp),(Xaf,Cc,YZfq),(Xaf,Cc,YZfr),(Xaf,Cc,YZfs),(Xaf,Cc,YZft),(Xaf,Cc,YZfu), (Xaf,Cc,YZfv),(Xaf,Cc,YZfw),(Xaf,Cc,YZfx),(Xaf,Cc,YZfy),(Xaf,Cc,YZfz),(Xaf, Cc,YZga),(Xaf,Cc,YZgb),(Xaf,Cc,YZgc),(Xaf,Cc,YZgd),(Xaf,Cc,YZge),(Xaf,Cc,YZgf), (Xaf,Cc,YZgg),(Xaf,Cc,YZgh),(Xaf,Cc,YZgi),(Xaf,Cc,YZgj),(Xaf,Cc,YZgk),(Xaf, Cc,YZgl),(Xaf,Cc,YZgm),(Xaf,Cc,YZgn),(Xaf,Cc,YZgo),(Xaf,Cc,YZgp),(Xaf,Cc,YZgq), (Xaf,Cc,YZgr),(Xaf,Cc,YZgs),(Xaf,Cc,YZgt),(Xaf,Cd,YZa),(Xaf,Cd,YZb),(Xaf,Cd, YZc),(Xaf,Cd,YZd),(Xaf,Cd,YZe),(Xaf,Cd,YZf),(Xaf,Cd,YZg),(Xaf,Cd,YZh),(Xaf, Cd,YZi),(Xaf,Cd,YZj),(Xaf,Cd,YZk),(Xaf,Cd,YZl),(Xaf,Cd,YZm),(Xaf,Cd,YZn),(Xaf, Cd,YZo),(Xaf,Cd,YZp),(Xaf,Cd,YZq),(Xaf,Cd,YZr),(Xaf,Cd,YZs),(Xaf,Cd,YZt),(Xaf, Cd,YZu),(Xaf,Cd,YZv),(Xaf,Cd,YZw),(Xaf,Cd,YZx),(Xaf,Cd,YZy),(Xaf,Cd,YZz),(Xaf, Cd,YZaa),(Xaf,Cd,YZab),(Xaf,Cd,YZac),(Xaf,Cd,YZad),(Xaf,Cd,YZae),(Xaf,Cd,YZaf), (Xaf,Cd,YZag),(Xaf,Cd,YZah),(Xaf,Cd,YZai),(Xaf,Cd,YZaj),(Xaf,Cd,YZak),(Xaf, Cd,YZal),(Xaf,Cd,YZam),(Xaf,Cd,YZan),(Xaf,Cd,YZao),(Xaf,Cd,YZap),(Xaf,Cd,YZaq), (Xaf,Cd,YZar),(Xaf,Cd,YZas),(Xaf,Cd,YZat),(Xaf,Cd,YZau),(Xaf,Cd,YZav),(Xaf, Cd,YZaw),(Xaf,Cd,YZax),(Xaf,Cd,YZay),(Xaf,Cd,YZaz),(Xaf,Cd,YZba),(Xaf,Cd,YZbb), (Xaf,Cd,YZbc),(Xaf,Cd,YZbd),(Xaf,Cd,YZbe),(Xaf,Cd,YZbf),(Xaf,Cd,YZbg),(Xaf, Cd,YZbh),(Xaf,Cd,YZbi),(Xaf,Cd,YZbj),(Xaf,Cd,YZbk),(Xaf,Cd,YZbl),(Xaf,Cd, YZbm),(Xaf,Cd,YZbn),(Xaf,Cd,YZbo),(Xaf,Cd,YZbp),(Xaf,Cd,YZbq),(Xaf,Cd,YZbr), (Xaf,Cd,YZbs),(Xaf,Cd,YZbt),(Xaf,Cd,YZbu),(Xaf,Cd,YZbv),(Xaf,Cd,YZbw),(Xaf,Cd, YZbx),(Xaf,Cd,YZby),(Xaf,Cd,YZbz),(Xaf,Cd,YZca),(Xaf,Cd,YZcb),(Xaf,Cd,YZcc), (Xaf,Cd,YZcd),(Xaf,Cd,YZce),(Xaf,Cd,YZcf),(Xaf,Cd,YZcg),(Xaf,Cd,YZch),(Xaf,Cd, YZci),(Xaf,Cd,YZcj),(Xaf,Cd,YZck),(Xaf,Cd,YZcl),(Xaf,Cd,YZcm),(Xaf,Cd,YZcn), (Xaf,Cd,YZco),(Xaf,Cd,YZcp),(Xaf,Cd,YZcq),(Xaf,Cd,YZcr),(Xaf,Cd,YZcs),(Xaf,Cd, YZct),(Xaf,Cd,YZcu),(Xaf,Cd,YZcv),(Xaf,Cd,YZcw),(Xaf,Cd,YZcx),(Xaf,Cd,YZcy), (Xaf,Cd,YZcz),(Xaf,Cd,YZda),(Xaf,Cd,YZdb),(Xaf,Cd,YZdc),(Xaf,Cd,YZdd),(Xaf, Cd,YZde),(Xaf,Cd,YZdf),(Xaf,Cd,YZdg),(Xaf,Cd,YZdh),(Xaf,Cd,YZdi),(Xaf,Cd,YZdj), (Xaf,Cd,YZdk),(Xaf,Cd,YZdl),(Xaf,Cd,YZdm),(Xaf,Cd,YZdn),(Xaf,Cd,YZdo),(Xaf, Cd,YZdp),(Xaf,Cd,YZdq),(Xaf,Cd,YZdr),(Xaf,Cd,YZds),(Xaf,Cd,Yzdt),(Xaf,Cd,YZdu), (Xaf,Cd,YZdv),(Xaf,Cd,YZdw),(Xaf,Cd,YZdx),(Xaf,Cd,YZdy),(Xaf,Cd,YZdz),(Xaf, Cd,YZea),(Xaf,Cd,YZeb),(Xaf,Cd,YZec),(Xaf,Cd,YZed),(Xaf,Cd,YZee),(Xaf,Cd,YZef), (Xaf,Cd,YZeg),(Xaf,Cd,YZeh),(Xaf,Cd,YZei),(Xaf,Cd,YZej),(Xaf,Cd,YZek),(Xaf, Cd,YZel),(Xaf,Cd,YZem),(Xaf,Cd,YZen),(Xaf,Cd,YZeo),(Xaf,Cd,YZep),(Xaf,Cd,YZeq), (Xaf,Cd,YZer),(Xaf,Cd,YZes),(Xaf,Cd,YZet),(Xaf,Cd,YZeu),(Xaf,Cd,YZev),(Xaf, Cd,YZew),(Xaf,Cd,YZex),(Xaf,Cd,YZey),(Xaf,Cd,YZez),(Xaf,Cd,YZfa),(Xaf,Cd,YZfb), (Xaf,Cd,YZfc),(Xaf,Cd,YZfd),(Xaf,Cd,YZfe),(Xaf,Cd,YZff),(Xaf,Cd,YZfg),(Xaf,Cd, YZfh),(Xaf,Cd,YZfi),(Xaf,Cd,YZfj),(Xaf,Cd,YZfk),(Xaf,Cd,YZfl),(Xaf,Cd,YZfm),(Xaf, Cd,YZfn),(Xaf,Cd,YZfo),(Xaf,Cd,YZfp),(Xaf,Cd,YZfq),(Xaf,Cd,YZfr),(Xaf,Cd,YZfs), (Xaf,Cd,YZft),(Xaf,Cd,YZfu),(Xaf,Cd,YZfv),(Xaf,Cd,YZfw),(Xaf,Cd,YZfx),(Xaf, Cd,YZfy),(Xaf,Cd,YZfz),(Xaf,Cd,YZga),(Xaf,Cd,YZgb),(Xaf,Cd,YZgc),(Xaf,Cd,YZgd), (Xaf,Cd,YZge),(Xaf,Cd,YZgf),(Xaf,Cd,YZgg),(Xaf,Cd,YZgh),(Xaf,Cd,YZgi),(Xaf, Cd,YZgj),(Xaf,Cd,YZgk),(Xaf,Cd,YZgl),(Xaf,Cd,YZgm),(Xaf,Cd,YZgn),(Xaf,Cd,YZgo), (Xaf,Cd,YZgp),(Xaf,Cd,YZgq),(Xaf,Cd,YZgr),(Xaf,Cd,YZgs),(Xaf,Cd,YZgt),(Xaf, Ce,YZa),(Xaf,Ce,YZb),(Xaf,Ce,YZc),(Xaf,Ce,YZd),(Xaf,Ce,YZe),(Xaf,Ce,YZf),(Xaf, Ce,YZg),(Xaf,Ce,YZh),(Xaf,Ce,YZi),(Xaf,Ce,YZj),(Xaf,Ce,YZk),(Xaf,Ce,YZl),(Xaf, Ce,YZm),(Xaf,Ce,YZn),(Xaf,Ce,YZo),(Xaf,Ce,YZp),(Xaf,Ce,YZq),(Xaf,Ce,YZr),(Xaf, Ce,YZs),(Xaf,Ce,YZt),(Xaf,Ce,YZu),(Xaf,Ce,YZv),(Xaf,Ce,YZw),(Xaf,Ce,YZx),(Xaf, Ce,YZy),(Xaf,Ce,YZz),(Xaf,Ce,YZaa),(Xaf,Ce,YZab),(Xaf,Ce,YZac),(Xaf,Ce,YZad), (Xaf,Ce,YZae),(Xaf,Ce,YZaf),(Xaf,Ce,YZag),(Xaf,Ce,YZah),(Xaf,Ce,YZai),(Xaf, Ce,YZaj),(Xaf,Ce,YZak),(Xaf,Ce,YZal),(Xaf,Ce,YZam),(Xaf,Ce,YZan),(Xaf,Ce,YZao), (Xaf,Ce,YZap),(Xaf,Ce,YZaq),(Xaf,Ce,YZar),(Xaf,Ce,YZas),(Xaf,Ce,YZat),(Xaf,Ce, YZau),(Xaf,Ce,YZav),(Xaf,Ce,YZaw),(Xaf,Ce,YZax),(Xaf,Ce,YZay),(Xaf,Ce,YZaz), (Xaf,Ce,YZba),(Xaf,Ce,YZbb),(Xaf,Ce,YZbc),(Xaf,Ce,YZbd),(Xaf,Ce,YZbe),(Xaf,Ce, YZbf),(Xaf,Ce,YZbg),(Xaf,Ce,YZbh),(Xaf,Ce,YZbi),(Xaf,Ce,YZbj),(Xaf,Ce,YZbk),(Xaf, Ce,YZbl),(Xaf,Ce,YZbm),(Xaf,Ce,YZbn),(Xaf,Ce,YZbo),(Xaf,Ce,YZbp),(Xaf,Ce, YZbq),(Xaf,Ce,YZbr),(Xaf,Ce,YZbs),(Xaf,Ce,YZbt),(Xaf,Ce,YZbu),(Xaf,Ce,YZbv),(Xaf, Ce,YZbw),(Xaf,Ce,YZbx),(Xaf,Ce,YZby),(Xaf,Ce,YZbz),(Xaf,Ce,YZca),(Xaf,Ce, YZcb),(Xaf,Ce,YZcc),(Xaf,Ce,YZcd),(Xaf,Ce,YZce),(Xaf,Ce,YZcf),(Xaf,Ce,YZcg),(Xaf, Ce,YZch),(Xaf,Ce,YZci),(Xaf,Ce,YZcj),(Xaf,Ce,YZck),(Xaf,Ce,YZcl),(Xaf,Ce,YZcm), (Xaf,Ce,YZcn),(Xaf,Ce,YZco),(Xaf,Ce,YZcp),(Xaf,Ce,YZcq),(Xaf,Ce,YZcr),(Xaf, Ce,YZcs),(Xaf,Ce,YZct),(Xaf,Ce,YZcu),(Xaf,Ce,YZcv),(Xaf,Ce,YZcw),(Xaf,Ce,YZcx), (Xaf,Ce,YZcy),(Xaf,Ce,YZcz),(Xaf,Ce,YZda),(Xaf,Ce,YZdb),(Xaf,Ce,YZdc),(Xaf, Ce,YZdd),(Xaf,Ce,YZde),(Xaf,Ce,YZdf),(Xaf,Ce,YZdg),(Xaf,Ce,YZdh),(Xaf,Ce,YZdi), (Xaf,Ce,YZdj),(Xaf,Ce,YZdk),(Xaf,Ce,YZdl),(Xaf,Ce,YZdm),(Xaf,Ce,YZdn),(Xaf, Ce,YZdo),(Xaf,Ce,YZdp),(Xaf,Ce,YZdq),(Xaf,Ce,YZdr),(Xaf,Ce,YZds),(Xaf,Ce,Yzdt), (Xaf,Ce,YZdu),(Xaf,Ce,YZdv),(Xaf,Ce,YZdw),(Xaf,Ce,YZdx),(Xaf,Ce,YZdy),(Xaf, Ce,YZdz),(Xaf,Ce,YZea),(Xaf,Ce,YZeb),(Xaf,Ce,YZec),(Xaf,Ce,YZed),(Xaf,Ce,YZee), (Xaf,Ce,YZef),(Xaf,Ce,YZeg),(Xaf,Ce,YZeh),(Xaf,Ce,YZei),(Xaf,Ce,YZej),(Xaf, Ce,YZek),(Xaf,Ce,YZel),(Xaf,Ce,YZem),(Xaf,Ce,YZen),(Xaf,Ce,YZeo),(Xaf,Ce,YZep), (Xaf,Ce,YZeq),(Xaf,Ce,YZer),(Xaf,Ce,YZes),(Xaf,Ce,YZet),(Xaf,Ce,YZeu),(Xaf,Ce, YZev),(Xaf,Ce,YZew),(Xaf,Ce,YZex),(Xaf,Ce,YZey),(Xaf,Ce,YZez),(Xaf,Ce,YZfa), (Xaf,Ce,YZfb),(Xaf,Ce,YZfc),(Xaf,Ce,YZfd),(Xaf,Ce,YZfe),(Xaf,Ce,YZff),(Xaf,Ce, YZfg),(Xaf,Ce,YZfh),(Xaf,Ce,YZfi),(Xaf,Ce,YZfj),(Xaf,Ce,YZfk),(Xaf,Ce,YZfl),(Xaf, Ce,YZfm),(Xaf,Ce,YZfn),(Xaf,Ce,YZfo),(Xaf,Ce,YZfp),(Xaf,Ce,YZfq),(Xaf,Ce,YZfr), (Xaf,Ce,YZfs),(Xaf,Ce,YZft),(Xaf,Ce,YZfu),(Xaf,Ce,YZfv),(Xaf,Ce,YZfw),(Xaf,Ce, YZfx),(Xaf,Ce,YZfy),(Xaf,Ce,YZfz),(Xaf,Ce,YZga),(Xaf,Ce,YZgb),(Xaf,Ce,YZgc),(Xaf, Ce,YZgd),(Xaf,Ce,YZge),(Xaf,Ce,YZgf),(Xaf,Ce,YZgg),(Xaf,Ce,YZgh),(Xaf,Ce, YZgi),(Xaf,Ce,YZgj),(Xaf,Ce,YZgk),(Xaf,Ce,YZgl),(Xaf,Ce,YZgm),(Xaf,Ce,YZgn),(Xaf, Ce,YZgo),(Xaf,Ce,YZgp),(Xaf,Ce,YZgq),(Xaf,Ce,YZgr),(Xaf,Ce,YZgs),(Xaf,Ce, YZgt),(Xaf,Cf,YZa),(Xaf,Cf,YZb),(Xaf,Cf,YZc),(Xaf,Cf,YZd),(Xaf,Cf,YZe),(Xaf,Cf, YZf),(Xaf,Cf,YZg),(Xaf,Cf,YZh),(Xaf,Cf,YZi),(Xaf,Cf,YZj),(Xaf,Cf,YZk),(Xaf,Cf,YZl), (Xaf,Cf,YZm),(Xaf,Cf,YZn),(Xaf,Cf,YZo),(Xaf,Cf,YZp),(Xaf,Cf,YZq),(Xaf,Cf,YZr), (Xaf,Cf,YZs),(Xaf,Cf,YZt),(Xaf,Cf,YZu),(Xaf,Cf,YZv),(Xaf,Cf,YZw),(Xaf,Cf,YZx), (Xaf,Cf,YZy),(Xaf,Cf,YZz),(Xaf,Cf,YZaa),(Xaf,Cf,YZab),(Xaf,Cf,YZac),(Xaf,Cf, YZad),(Xaf,Cf,YZae),(Xaf,Cf,YZaf),(Xaf,Cf,YZag),(Xaf,Cf,YZah),(Xaf,Cf,YZai),(Xaf, Cf,YZaj),(Xaf,Cf,YZak),(Xaf,Cf,YZal),(Xaf,Cf,YZam),(Xaf,Cf,YZan),(Xaf,Cf,YZao), (Xaf,Cf,YZap),(Xaf,Cf,YZaq),(Xaf,Cf,YZar),(Xaf,Cf,YZas),(Xaf,Cf,YZat),(Xaf,Cf, YZau),(Xaf,Cf,YZav),(Xaf,Cf,YZaw),(Xaf,Cf,YZax),(Xaf,Cf,YZay),(Xaf,Cf,YZaz),(Xaf, Cf,YZba),(Xaf,Cf,YZbb),(Xaf,Cf,YZbc),(Xaf,Cf,YZbd),(Xaf,Cf,YZbe),(Xaf,Cf,YZbf), (Xaf,Cf,YZbg),(Xaf,Cf,YZbh),(Xaf,Cf,YZbi),(Xaf,Cf,YZbj),(Xaf,Cf,YZbk),(Xaf, Cf,YZbl),(Xaf,Cf,YZbm),(Xaf,Cf,YZbn),(Xaf,Cf,YZbo),(Xaf,Cf,YZbp),(Xaf,Cf,YZbq), (Xaf,Cf,YZbr),(Xaf,Cf,YZbs),(Xaf,Cf,YZbt),(Xaf,Cf,YZbu),(Xaf,Cf,YZbv),(Xaf,Cf, YZbw),(Xaf,Cf,YZbx),(Xaf,Cf,YZby),(Xaf,Cf,YZbz),(Xaf,Cf,YZca),(Xaf,Cf,YZcb),(Xaf, Cf,YZcc),(Xaf,Cf,YZcd),(Xaf,Cf,YZce),(Xaf,Cf,YZcf),(Xaf,Cf,YZcg),(Xaf,Cf,YZch), (Xaf,Cf,YZci),(Xaf,Cf,YZcj),(Xaf,Cf,YZck),(Xaf,Cf,YZcl),(Xaf,Cf,YZcm),(Xaf, Cf,YZcn),(Xaf,Cf,YZco),(Xaf,Cf,YZcp),(Xaf,Cf,YZcq),(Xaf,Cf,YZcr),(Xaf,Cf,YZcs), (Xaf,Cf,YZct),(Xaf,Cf,YZcu),(Xaf,Cf,YZcv),(Xaf,Cf,YZcw),(Xaf,Cf,YZcx),(Xaf,Cf, YZcy),(Xaf,Cf,YZcz),(Xaf,Cf,YZda),(Xaf,Cf,YZdb),(Xaf,Cf,YZdc),(Xaf,Cf,YZdd),(Xaf, Cf,YZde),(Xaf,Cf,YZdf),(Xaf,Cf,YZdg),(Xaf,Cf,YZdh),(Xaf,Cf,YZdi),(Xaf,Cf,YZdj), (Xaf,Cf,YZdk),(Xaf,Cf,YZdl),(Xaf,Cf,YZdm),(Xaf,Cf,YZdn),(Xaf,Cf,YZdo),(Xaf,Cf, YZdp),(Xaf,Cf,YZdq),(Xaf,Cf,YZdr),(Xaf,Cf,YZds),(Xaf,Cf,Yzdt),(Xaf,Cf,YZdu),(Xaf, Cf,YZdv),(Xaf,Cf,YZdw),(Xaf,Cf,YZdx),(Xaf,Cf,YZdy),(Xaf,Cf,YZdz),(Xaf,Cf, YZea),(Xaf,Cf,YZeb),(Xaf,Cf,YZec),(Xaf,Cf,YZed),(Xaf,Cf,YZee),(Xaf,Cf,YZef),(Xaf, Cf,YZeg),(Xaf,Cf,YZeh),(Xaf,Cf,YZei),(Xaf,Cf,YZej),(Xaf,Cf,YZek),(Xaf,Cf,YZel), (Xaf,Cf,YZem),(Xaf,Cf,YZen),(Xaf,Cf,YZeo),(Xaf,Cf,YZep),(Xaf,Cf,YZeq),(Xaf,Cf, YZer),(Xaf,Cf,YZes),(Xaf,Cf,YZet),(Xaf,Cf,YZeu),(Xaf,Cf,YZev),(Xaf,Cf,YZew),(Xaf, Cf,YZex),(Xaf,Cf,YZey),(Xaf,Cf,YZez),(Xaf,Cf,YZfa),(Xaf,Cf,YZfb),(Xaf,Cf,YZfc), (Xaf,Cf,YZfd),(Xaf,Cf,YZfe),(Xaf,Cf,YZff),(Xaf,Cf,YZfg),(Xaf,Cf,YZfh),(Xaf,Cf, YZfi),(Xaf,Cf,YZfj),(Xaf,Cf,YZfk),(Xaf,Cf,YZfl),(Xaf,Cf,YZfm),(Xaf,Cf,YZfn),(Xaf, Cf,YZfo),(Xaf,Cf,YZfp),(Xaf,Cf,YZfq),(Xaf,Cf,YZfr),(Xaf,Cf,YZfs),(Xaf,Cf,YZft),(Xaf, Cf,YZfu),(Xaf,Cf,YZfv),(Xaf,Cf,YZfw),(Xaf,Cf,YZfx),(Xaf,Cf,YZfy),(Xaf,Cf,YZfz), (Xaf,Cf,YZga),(Xaf,Cf,YZgb),(Xaf,Cf,YZgc),(Xaf,Cf,YZgd),(Xaf,Cf,YZge),(Xaf,Cf, YZgf),(Xaf,Cf,YZgg),(Xaf,Cf,YZgh),(Xaf,Cf,YZgi),(Xaf,Cf,YZgj),(Xaf,Cf,YZgk),(Xaf, Cf,YZgl),(Xaf,Cf,YZgm),(Xaf,Cf,YZgn),(Xaf,Cf,YZgo),(Xaf,Cf,YZgp),(Xaf,Cf, YZgq),(Xaf,Cf,YZgr),(Xaf,Cf,YZgs),(Xaf,Cf,YZgt), (Xag,Ca,YZa),(Xag,Ca,YZb),(Xag,Ca,YZc),(Xag,Ca,YZd),(Xag,Ca,YZe),(Xag,Ca,YZf), (Xag,Ca,YZg),(Xag,Ca,YZh),(Xag,Ca,YZi),(Xag,Ca,YZj),(Xag,Ca,YZk),(Xag,Ca,YZl), (Xag,Ca,YZm),(Xag,Ca,YZn),(Xag,Ca,YZo),(Xag,Ca,YZp),(Xag,Ca,YZq),(Xag,Ca, YZr),(Xag,Ca,YZs),(Xag,Ca,YZt),(Xag,Ca,YZu),(Xag,Ca,YZv),(Xag,Ca,YZw),(Xag, Ca,YZx),(Xag,Ca,YZy),(Xag,Ca,YZz),(Xag,Ca,YZaa),(Xag,Ca,YZab),(Xag,Ca,YZac), (Xag,Ca,YZad),(Xag,Ca,YZae),(Xag,Ca,YZaf),(Xag,Ca,YZag),(Xag,Ca,YZah),(Xag, Ca,YZai),(Xag,Ca,YZaj),(Xag,Ca,YZak),(Xag,Ca,YZal),(Xag,Ca,YZam),(Xag,Ca,YZan), (Xag,Ca,YZao),(Xag,Ca,YZap),(Xag,Ca,YZaq),(Xag,Ca,YZar),(Xag,Ca,YZas),(Xag, Ca,YZat),(Xag,Ca,YZau),(Xag,Ca,YZav),(Xag,Ca,YZaw),(Xag,Ca,YZax),(Xag,Ca, YZay),(Xag,Ca,YZaz),(Xag,Ca,YZba),(Xag,Ca,YZbb),(Xag,Ca,YZbc),(Xag,Ca,YZbd), (Xag,Ca,YZbe),(Xag,Ca,YZbf),(Xag,Ca,YZbg),(Xag,Ca,YZbh),(Xag,Ca,YZbi),(Xag, Ca,YZbj),(Xag,Ca,YZbk),(Xag,Ca,YZbl),(Xag,Ca,YZbm),(Xag,Ca,YZbn),(Xag,Ca,YZbo), (Xag,Ca,YZbp),(Xag,Ca,YZbq),(Xag,Ca,YZbr),(Xag,Ca,YZbs),(Xag,Ca,YZbt),(Xag, Ca,YZbu),(Xag,Ca,YZbv),(Xag,Ca,YZbw),(Xag,Ca,YZbx),(Xag,Ca,YZby),(Xag, Ca,YZbz),(Xag,Ca,YZca),(Xag,Ca,YZcb),(Xag,Ca,YZcc),(Xag,Ca,YZcd),(Xag,Ca,YZce), (Xag,Ca,YZcf),(Xag,Ca,YZcg),(Xag,Ca,YZch),(Xag,Ca,YZci),(Xag,Ca,YZcj),(Xag, Ca,YZck),(Xag,Ca,YZcl),(Xag,Ca,YZcm),(Xag,Ca,YZcn),(Xag,Ca,YZco),(Xag,Ca, YZcp),(Xag,Ca,YZcq),(Xag,Ca,YZcr),(Xag,Ca,YZcs),(Xag,Ca,YZct),(Xag,Ca,YZcu), (Xag,Ca,YZcv),(Xag,Ca,YZcw),(Xag,Ca,YZcx),(Xag,Ca,YZcy),(Xag,Ca,YZcz),(Xag, Ca,YZda),(Xag,Ca,YZdb),(Xag,Ca,YZdc),(Xag,Ca,YZdd),(Xag,Ca,YZde),(Xag,Ca,YZdf), (Xag,Ca,YZdg),(Xag,Ca,YZdh),(Xag,Ca,YZdi),(Xag,Ca,YZdj),(Xag,Ca,YZdk),(Xag, Ca,YZdl),(Xag,Ca,YZdm),(Xag,Ca,YZdn),(Xag,Ca,YZdo),(Xag,Ca,YZdp),(Xag, Ca,YZdq),(Xag,Ca,YZdr),(Xag,Ca,YZds),(Xag,Ca,Yzdt),(Xag,Ca,YZdu),(Xag,Ca,YZdv), (Xag,Ca,YZdw),(Xag,Ca,YZdx),(Xag,Ca,YZdy),(Xag,Ca,YZdz),(Xag,Ca,YZea),(Xag, Ca,YZeb),(Xag,Ca,YZec),(Xag,Ca,YZed),(Xag,Ca,YZee),(Xag,Ca,YZef),(Xag,Ca, YZeg),(Xag,Ca,YZeh),(Xag,Ca,YZei),(Xag,Ca,YZej),(Xag,Ca,YZek),(Xag,Ca,YZel), (Xag,Ca,YZem),(Xag,Ca,YZen),(Xag,Ca,YZeo),(Xag,Ca,YZep),(Xag,Ca,YZeq),(Xag, Ca,YZer),(Xag,Ca,YZes),(Xag,Ca,YZet),(Xag,Ca,YZeu),(Xag,Ca,YZev),(Xag,Ca,YZew), (Xag,Ca,YZex),(Xag,Ca,YZey),(Xag,Ca,YZez),(Xag,Ca,YZfa),(Xag,Ca,YZfb),(Xag, Ca,YZfc),(Xag,Ca,YZfd),(Xag,Ca,YZfe),(Xag,Ca,YZff),(Xag,Ca,YZfg),(Xag,Ca,YZfh), (Xag,Ca,YZfi),(Xag,Ca,YZfj),(Xag,Ca,YZfk),(Xag,Ca,YZfl),(Xag,Ca,YZfm),(Xag, Ca,YZfn),(Xag,Ca,YZfo),(Xag,Ca,YZfp),(Xag,Ca,YZfq),(Xag,Ca,YZfr),(Xag,Ca,YZfs), (Xag,Ca,YZft),(Xag,Ca,YZfu),(Xag,Ca,YZfv),(Xag,Ca,YZfw),(Xag,Ca,YZfx),(Xag, Ca,YZfy),(Xag,Ca,YZfz),(Xag,Ca,YZga),(Xag,Ca,YZgb),(Xag,Ca,YZgc),(Xag,Ca, YZgd),(Xag,Ca,YZge),(Xag,Ca,YZgf),(Xag,Ca,YZgg),(Xag,Ca,YZgh),(Xag,Ca,YZgi), (Xag,Ca,YZgj),(Xag,Ca,YZgk),(Xag,Ca,YZgl),(Xag,Ca,YZgm),(Xag,Ca,YZgn),(Xag, Ca,YZgo),(Xag,Ca,YZgp),(Xag,Ca,YZgq),(Xag,Ca,YZgr),(Xag,Ca,YZgs),(Xag,Ca,YZgt), (Xag,Cb,YZa),(Xag,Cb,YZb),(Xag,Cb,YZc),(Xag,Cb,YZd),(Xag,Cb,YZe),(Xag, Cb,YZf),(Xag,Cb,YZg),(Xag,Cb,YZh),(Xag,Cb,YZi),(Xag,Cb,YZj),(Xag,Cb,YZk),(Xag, Cb,YZl),(Xag,Cb,YZm),(Xag,Cb,YZn),(Xag,Cb,YZo),(Xag,Cb,YZp),(Xag,Cb,YZq), (Xag,Cb,YZr),(Xag,Cb,YZs),(Xag,Cb,YZt),(Xag,Cb,YZu),(Xag,Cb,YZv),(Xag,Cb,YZw), (Xag,Cb,YZx),(Xag,Cb,YZy),(Xag,Cb,YZz),(Xag,Cb,YZaa),(Xag,Cb,YZab),(Xag, Cb,YZac),(Xag,Cb,YZad),(Xag,Cb,YZae),(Xag,Cb,YZaf),(Xag,Cb,YZag),(Xag,Cb, YZah),(Xag,Cb,YZai),(Xag,Cb,YZaj),(Xag,Cb,YZak),(Xag,Cb,YZal),(Xag,Cb,YZam), (Xag,Cb,YZan),(Xag,Cb,YZao),(Xag,Cb,YZap),(Xag,Cb,YZaq),(Xag,Cb,YZar),(Xag, Cb,YZas),(Xag,Cb,YZat),(Xag,Cb,YZau),(Xag,Cb,YZav),(Xag,Cb,YZaw),(Xag,Cb,YZax), (Xag,Cb,YZay),(Xag,Cb,YZaz),(Xag,Cb,YZba),(Xag,Cb,YZbb),(Xag,Cb,YZbc), (Xag,Cb,YZbd),(Xag,Cb,YZbe),(Xag,Cb,YZbf),(Xag,Cb,YZbg),(Xag,Cb,YZbh),(Xag, Cb,YZbi),(Xag,Cb,YZbj),(Xag,Cb,YZbk),(Xag,Cb,YZbl),(Xag,Cb,YZbm),(Xag,Cb,YZbn), (Xag,Cb,YZbo),(Xag,Cb,YZbp),(Xag,Cb,YZbq),(Xag,Cb,YZbr),(Xag,Cb,YZbs), (Xag,Cb,YZbt),(Xag,Cb,YZbu),(Xag,Cb,YZbv),(Xag,Cb,YZbw),(Xag,Cb,YZbx),(Xag, Cb,YZby),(Xag,Cb,YZbz),(Xag,Cb,YZca),(Xag,Cb,YZcb),(Xag,Cb,YZcc),(Xag,Cb,YZcd), (Xag,Cb,YZce),(Xag,Cb,YZcf),(Xag,Cb,YZcg),(Xag,Cb,YZch),(Xag,Cb,YZci),(Xag, Cb,YZcj),(Xag,Cb,YZck),(Xag,Cb,YZcl),(Xag,Cb,YZcm),(Xag,Cb,YZcn),(Xag, Cb,YZco),(Xag,Cb,YZcp),(Xag,Cb,YZcq),(Xag,Cb,YZcr),(Xag,Cb,YZcs),(Xag,Cb,YZct), (Xag,Cb,YZcu),(Xag,Cb,YZcv),(Xag,Cb,YZcw),(Xag,Cb,YZcx),(Xag,Cb,YZcy), (Xag,Cb,YZcz),(Xag,Cb,YZda),(Xag,Cb,YZdb),(Xag,Cb,YZdc),(Xag,Cb,YZdd),(Xag, Cb,YZde),(Xag,Cb,YZdf),(Xag,Cb,YZdg),(Xag,Cb,YZdh),(Xag,Cb,YZdi),(Xag,Cb,YZdj), (Xag,Cb,YZdk),(Xag,Cb,YZdl),(Xag,Cb,YZdm),(Xag,Cb,YZdn),(Xag,Cb,YZdo), (Xag,Cb,YZdp),(Xag,Cb,YZdq),(Xag,Cb,YZdr),(Xag,Cb,YZds),(Xag,Cb,Yzdt),(Xag, Cb,YZdu),(Xag,Cb,YZdv),(Xag,Cb,YZdw),(Xag,Cb,YZdx),(Xag,Cb,YZdy),(Xag,Cb, YZdz),(Xag,Cb,YZea),(Xag,Cb,YZeb),(Xag,Cb,YZec),(Xag,Cb,YZed),(Xag,Cb,YZee), (Xag,Cb,YZef),(Xag,Cb,YZeg),(Xag,Cb,YZeh),(Xag,Cb,YZei),(Xag,Cb,YZej),(Xag, Cb,YZek),(Xag,Cb,YZel),(Xag,Cb,YZem),(Xag,Cb,YZen),(Xag,Cb,YZeo),(Xag,Cb,YZep), (Xag,Cb,YZeq),(Xag,Cb,YZer),(Xag,Cb,YZes),(Xag,Cb,YZet),(Xag,Cb,YZeu),(Xag, Cb,YZev),(Xag,Cb,YZew),(Xag,Cb,YZex),(Xag,Cb,YZey),(Xag,Cb,YZez),(Xag, Cb,YZfa),(Xag,Cb,YZfb),(Xag,Cb,YZfc),(Xag,Cb,YZfd),(Xag,Cb,YZfe),(Xag,Cb,YZff), (Xag,Cb,YZfg),(Xag,Cb,YZfh),(Xag,Cb,YZfi),(Xag,Cb,YZfj),(Xag,Cb,YZfk),(Xag, Cb,YZfl),(Xag,Cb,YZfm),(Xag,Cb,YZfn),(Xag,Cb,YZfo),(Xag,Cb,YZfp),(Xag,Cb,YZfq), (Xag,Cb,YZfr),(Xag,Cb,YZfs),(Xag,Cb,YZft),(Xag,Cb,YZfu),(Xag,Cb,YZfv),(Xag, Cb,YZfw),(Xag,Cb,YZfx),(Xag,Cb,YZfy),(Xag,Cb,YZfz),(Xag,Cb,YZga),(Xag,Cb,YZgb), (Xag,Cb,YZgc),(Xag,Cb,YZgd),(Xag,Cb,YZge),(Xag,Cb,YZgf),(Xag,Cb,YZgg), (Xag,Cb,YZgh),(Xag,Cb,YZgi),(Xag,Cb,YZgj),(Xag,Cb,YZgk),(Xag,Cb,YZgl),(Xag, Cb,YZgm),(Xag,Cb,YZgn),(Xag,Cb,YZgo),(Xag,Cb,YZgp),(Xag,Cb,YZgq),(Xag,Cb, YZgr),(Xag,Cb,YZgs),(Xag,Cb,YZgt),(Xag,Cc,YZa),(Xag,Cc,YZb),(Xag,Cc,YZc),(Xag, Cc,YZd),(Xag,Cc,YZe),(Xag,Cc,YZf),(Xag,Cc,YZg),(Xag,Cc,YZh),(Xag,Cc,YZi),(Xag, Cc,YZj),(Xag,Cc,YZk),(Xag,Cc,YZl),(Xag,Cc,YZm),(Xag,Cc,YZn),(Xag,Cc,YZo), (Xag,Cc,YZp),(Xag,Cc,YZq),(Xag,Cc,YZr),(Xag,Cc,YZs),(Xag,Cc,YZt),(Xag,Cc, YZu),(Xag,Cc,YZv),(Xag,Cc,YZw),(Xag,Cc,YZx),(Xag,Cc,YZy),(Xag,Cc,YZz),(Xag, Cc,YZaa),(Xag,Cc,YZab),(Xag,Cc,YZac),(Xag,Cc,YZad),(Xag,Cc,YZae),(Xag,Cc,YZaf), (Xag,Cc,YZag),(Xag,Cc,YZah),(Xag,Cc,YZai),(Xag,Cc,YZaj),(Xag,Cc,YZak),(Xag, Cc,YZal),(Xag,Cc,YZam),(Xag,Cc,YZan),(Xag,Cc,YZao),(Xag,Cc,YZap),(Xag,Cc, YZaq),(Xag,Cc,YZar),(Xag,Cc,YZas),(Xag,Cc,YZat),(Xag,Cc,YZau),(Xag,Cc,YZav), (Xag,Cc,YZaw),(Xag,Cc,YZax),(Xag,Cc,YZay),(Xag,Cc,YZaz),(Xag,Cc,YZba),(Xag, Cc,YZbb),(Xag,Cc,YZbc),(Xag,Cc,YZbd),(Xag,Cc,YZbe),(Xag,Cc,YZbf),(Xag,Cc,YZbg), (Xag,Cc,YZbh),(Xag,Cc,YZbi),(Xag,Cc,YZbj),(Xag,Cc,YZbk),(Xag,Cc,YZbl),(Xag, Cc,YZbm),(Xag,Cc,YZbn),(Xag,Cc,YZbo),(Xag,Cc,YZbp),(Xag,Cc,YZbq),(Xag, Cc,YZbr),(Xag,Cc,YZbs),(Xag,Cc,YZbt),(Xag,Cc,YZbu),(Xag,Cc,YZbv),(Xag,Cc,YZbw), (Xag,Cc,YZbx),(Xag,Cc,YZby),(Xag,Cc,YZbz),(Xag,Cc,YZca),(Xag,Cc,YZcb),(Xag, Cc,YZcc),(Xag,Cc,YZcd),(Xag,Cc,YZce),(Xag,Cc,YZcf),(Xag,Cc,YZcg),(Xag,Cc, YZch),(Xag,Cc,YZci),(Xag,Cc,YZcj),(Xag,Cc,YZck),(Xag,Cc,YZcl),(Xag,Cc,YZcm), (Xag,Cc,YZcn),(Xag,Cc,YZco),(Xag,Cc,YZcp),(Xag,Cc,YZcq),(Xag,Cc,YZcr),(Xag, Cc,YZcs),(Xag,Cc,YZct),(Xag,Cc,YZcu),(Xag,Cc,YZcv),(Xag,Cc,YZcw),(Xag,Cc,YZcx), (Xag,Cc,YZcy),(Xag,Cc,YZcz),(Xag,Cc,YZda),(Xag,Cc,YZdb),(Xag,Cc,YZdc),(Xag, Cc,YZdd),(Xag,Cc,YZde),(Xag,Cc,YZdf),(Xag,Cc,YZdg),(Xag,Cc,YZdh),(Xag,Cc, YZdi),(Xag,Cc,YZdj),(Xag,Cc,YZdk),(Xag,Cc,YZdl),(Xag,Cc,YZdm),(Xag,Cc,YZdn), (Xag,Cc,YZdo),(Xag,Cc,YZdp),(Xag,Cc,YZdq),(Xag,Cc,YZdr),(Xag,Cc,YZds),(Xag, Cc,Yzdt),(Xag,Cc,YZdu),(Xag,Cc,YZdv),(Xag,Cc,YZdw),(Xag,Cc,YZdx),(Xag,Cc, YZdy),(Xag,Cc,YZdz),(Xag,Cc,YZea),(Xag,Cc,YZeb),(Xag,Cc,YZec),(Xag,Cc,YZed), (Xag,Cc,YZee),(Xag,Cc,YZef),(Xag,Cc,YZeg),(Xag,Cc,YZeh),(Xag,Cc,YZei),(Xag, Cc,YZej),(Xag,Cc,YZek),(Xag,Cc,YZel),(Xag,Cc,YZem),(Xag,Cc,YZen),(Xag,Cc,YZeo), (Xag,Cc,YZep),(Xag,Cc,YZeq),(Xag,Cc,YZer),(Xag,Cc,YZes),(Xag,Cc,YZet),(Xag, Cc,YZeu),(Xag,Cc,YZev),(Xag,Cc,YZew),(Xag,Cc,YZex),(Xag,Cc,YZey),(Xag,Cc, YZez),(Xag,Cc,YZfa),(Xag,Cc,YZfb),(Xag,Cc,YZfc),(Xag,Cc,YZfd),(Xag,Cc,YZfe),(Xag, Cc,YZff),(Xag,Cc,YZfg),(Xag,Cc,YZfh),(Xag,Cc,YZfi),(Xag,Cc,YZfj),(Xag,Cc, YZfk),(Xag,Cc,YZfl),(Xag,Cc,YZfm),(Xag,Cc,YZfn),(Xag,Cc,YZfo),(Xag,Cc,YZfp),(Xag, Cc,YZfq),(Xag,Cc,YZfr),(Xag,Cc,YZfs),(Xag,Cc,YZft),(Xag,Cc,YZfu),(Xag,Cc, YZfv),(Xag,Cc,YZfw),(Xag,Cc,YZfx),(Xag,Cc,YZfy),(Xag,Cc,YZfz),(Xag,Cc,YZga), (Xag,Cc,YZgb),(Xag,Cc,YZgc),(Xag,Cc,YZgd),(Xag,Cc,YZge),(Xag,Cc,YZgf),(Xag, Cc,YZgg),(Xag,Cc,YZgh),(Xag,Cc,YZgi),(Xag,Cc,YZgj),(Xag,Cc,YZgk),(Xag,Cc,YZgl), (Xag,Cc,YZgm),(Xag,Cc,YZgn),(Xag,Cc,YZgo),(Xag,Cc,YZgp),(Xag,Cc,YZgq),(Xag, Cc,YZgr),(Xag,Cc,YZgs),(Xag,Cc,YZgt),(Xag,Cd,YZa),(Xag,Cd,YZb),(Xag,Cd, YZc),(Xag,Cd,YZd),(Xag,Cd,YZe),(Xag,Cd,YZf),(Xag,Cd,YZg),(Xag,Cd,YZh),(Xag, Cd,YZi),(Xag,Cd,YZj),(Xag,Cd,YZk),(Xag,Cd,YZl),(Xag,Cd,YZm),(Xag,Cd,YZn),(Xag, Cd,YZo),(Xag,Cd,YZp),(Xag,Cd,YZq),(Xag,Cd,YZr),(Xag,Cd,YZs),(Xag,Cd,YZt), (Xag,Cd,YZu),(Xag,Cd,YZv),(Xag,Cd,YZw),(Xag,Cd,YZx),(Xag,Cd,YZy),(Xag,Cd, YZz),(Xag,Cd,YZaa),(Xag,Cd,YZab),(Xag,Cd,YZac),(Xag,Cd,YZad),(Xag,Cd,YZae), (Xag,Cd,YZaf),(Xag,Cd,YZag),(Xag,Cd,YZah),(Xag,Cd,YZai),(Xag,Cd,YZaj),(Xag,Cd, YZak),(Xag,Cd,YZal),(Xag,Cd,YZam),(Xag,Cd,YZan),(Xag,Cd,YZao),(Xag,Cd,YZap), (Xag,Cd,YZaq),(Xag,Cd,YZar),(Xag,Cd,YZas),(Xag,Cd,YZat),(Xag,Cd,YZau),(Xag, Cd,YZav),(Xag,Cd,YZaw),(Xag,Cd,YZax),(Xag,Cd,YZay),(Xag,Cd,YZaz),(Xag,Cd, YZba),(Xag,Cd,YZbb),(Xag,Cd,YZbc),(Xag,Cd,YZbd),(Xag,Cd,YZbe),(Xag,Cd,YZbf), (Xag,Cd,YZbg),(Xag,Cd,YZbh),(Xag,Cd,YZbi),(Xag,Cd,YZbj),(Xag,Cd,YZbk),(Xag, Cd,YZbl),(Xag,Cd,YZbm),(Xag,Cd,YZbn),(Xag,Cd,YZbo),(Xag,Cd,YZbp),(Xag,Cd, YZbq),(Xag,Cd,YZbr),(Xag,Cd,YZbs),(Xag,Cd,YZbt),(Xag,Cd,YZbu),(Xag,Cd,YZbv), (Xag,Cd,YZbw),(Xag,Cd,YZbx),(Xag,Cd,YZby),(Xag,Cd,YZbz),(Xag,Cd,YZca),(Xag, Cd,YZcb),(Xag,Cd,YZcc),(Xag,Cd,YZcd),(Xag,Cd,YZce),(Xag,Cd,YZcf),(Xag,Cd, YZcg),(Xag,Cd,YZch),(Xag,Cd,YZci),(Xag,Cd,YZcj),(Xag,Cd,YZck),(Xag,Cd,YZcl), (Xag,Cd,YZcm),(Xag,Cd,YZcn),(Xag,Cd,YZco),(Xag,Cd,YZcp),(Xag,Cd,YZcq),(Xag, Cd,YZcr),(Xag,Cd,YZcs),(Xag,Cd,YZct),(Xag,Cd,YZcu),(Xag,Cd,YZcv),(Xag,Cd, YZcw),(Xag,Cd,YZcx),(Xag,Cd,YZcy),(Xag,Cd,YZcz),(Xag,Cd,YZda),(Xag,Cd,YZdb), (Xag,Cd,YZdc),(Xag,Cd,YZdd),(Xag,Cd,YZde),(Xag,Cd,YZdf),(Xag,Cd,YZdg),(Xag, Cd,YZdh),(Xag,Cd,YZdi),(Xag,Cd,YZdj),(Xag,Cd,YZdk),(Xag,Cd,YZdl),(Xag,Cd, YZdm),(Xag,Cd,YZdn),(Xag,Cd,YZdo),(Xag,Cd,YZdp),(Xag,Cd,YZdq),(Xag,Cd,YZdr), (Xag,Cd,YZds),(Xag,Cd,Yzdt),(Xag,Cd,YZdu),(Xag,Cd,YZdv),(Xag,Cd,YZdw),(Xag, Cd,YZdx),(Xag,Cd,YZdy),(Xag,Cd,YZdz),(Xag,Cd,YZea),(Xag,Cd,YZeb),(Xag, Cd,YZec),(Xag,Cd,YZed),(Xag,Cd,YZee),(Xag,Cd,YZef),(Xag,Cd,YZeg),(Xag,Cd,YZeh), (Xag,Cd,YZei),(Xag,Cd,YZej),(Xag,Cd,YZek),(Xag,Cd,YZel),(Xag,Cd,YZem),(Xag, Cd,YZen),(Xag,Cd,YZeo),(Xag,Cd,YZep),(Xag,Cd,YZeq),(Xag,Cd,YZer),(Xag, Cd,YZes),(Xag,Cd,YZet),(Xag,Cd,YZeu),(Xag,Cd,YZev),(Xag,Cd,YZew),(Xag,Cd,YZex), (Xag,Cd,YZey),(Xag,Cd,YZez),(Xag,Cd,YZfa),(Xag,Cd,YZfb),(Xag,Cd,YZfc),(Xag, Cd,YZfd),(Xag,Cd,YZfe),(Xag,Cd,YZff),(Xag,Cd,YZfg),(Xag,Cd,YZfh),(Xag,Cd, YZfi),(Xag,Cd,YZfj),(Xag,Cd,YZfk),(Xag,Cd,YZfl),(Xag,Cd,YZfm),(Xag,Cd,YZfn), (Xag,Cd,YZfo),(Xag,Cd,YZfp),(Xag,Cd,YZfq),(Xag,Cd,YZfr),(Xag,Cd,YZfs),(Xag,Cd, YZft),(Xag,Cd,YZfu),(Xag,Cd,YZfv),(Xag,Cd,YZfw),(Xag,Cd,YZfx),(Xag,Cd,YZfy), (Xag,Cd,YZfz),(Xag,Cd,YZga),(Xag,Cd,YZgb),(Xag,Cd,YZgc),(Xag,Cd,YZgd),(Xag, Cd,YZge),(Xag,Cd,YZgf),(Xag,Cd,YZgg),(Xag,Cd,YZgh),(Xag,Cd,YZgi),(Xag,Cd, YZgj),(Xag,Cd,YZgk),(Xag,Cd,YZgl),(Xag,Cd,YZgm),(Xag,Cd,YZgn),(Xag,Cd,YZgo), (Xag,Cd,YZgp),(Xag,Cd,YZgq),(Xag,Cd,YZgr),(Xag,Cd,YZgs),(Xag,Cd,YZgt),(Xag, Ce,YZa),(Xag,Ce,YZb),(Xag,Ce,YZc),(Xag,Ce,YZd),(Xag,Ce,YZe),(Xag,Ce,YZf),(Xag, Ce,YZg),(Xag,Ce,YZh),(Xag,Ce,YZi),(Xag,Ce,YZj),(Xag,Ce,YZk),(Xag,Ce,YZl), (Xag,Ce,YZm),(Xag,Ce,YZn),(Xag,Ce,YZo),(Xag,Ce,YZp),(Xag,Ce,YZq),(Xag,Ce,YZr), (Xag,Ce,YZs),(Xag,Ce,YZt),(Xag,Ce,YZu),(Xag,Ce,YZv),(Xag,Ce,YZw),(Xag,Ce, YZx),(Xag,Ce,YZy),(Xag,Ce,YZz),(Xag,Ce,YZaa),(Xag,Ce,YZab),(Xag,Ce,YZac),(Xag, Ce,YZad),(Xag,Ce,YZae),(Xag,Ce,YZaf),(Xag,Ce,YZag),(Xag,Ce,YZah),(Xag,Ce, YZai),(Xag,Ce,YZaj),(Xag,Ce,YZak),(Xag,Ce,YZal),(Xag,Ce,YZam),(Xag,Ce,YZan), (Xag,Ce,YZao),(Xag,Ce,YZap),(Xag,Ce,YZaq),(Xag,Ce,YZar),(Xag,Ce,YZas),(Xag, Ce,YZat),(Xag,Ce,YZau),(Xag,Ce,YZav),(Xag,Ce,YZaw),(Xag,Ce,YZax),(Xag,Ce,YZay), (Xag,Ce,YZaz),(Xag,Ce,YZba),(Xag,Ce,YZbb),(Xag,Ce,YZbc),(Xag,Ce,YZbd),(Xag, Ce,YZbe),(Xag,Ce,YZbf),(Xag,Ce,YZbg),(Xag,Ce,YZbh),(Xag,Ce,YZbi),(Xag,Ce, YZbj),(Xag,Ce,YZbk),(Xag,Ce,YZbl),(Xag,Ce,YZbm),(Xag,Ce,YZbn),(Xag,Ce,YZbo), (Xag,Ce,YZbp),(Xag,Ce,YZbq),(Xag,Ce,YZbr),(Xag,Ce,YZbs),(Xag,Ce,YZbt),(Xag, Ce,YZbu),(Xag,Ce,YZbv),(Xag,Ce,YZbw),(Xag,Ce,YZbx),(Xag,Ce,YZby),(Xag,Ce, YZbz),(Xag,Ce,YZca),(Xag,Ce,YZcb),(Xag,Ce,YZcc),(Xag,Ce,YZcd),(Xag,Ce,YZce), (Xag,Ce,YZcf),(Xag,Ce,YZcg),(Xag,Ce,YZch),(Xag,Ce,YZci),(Xag,Ce,YZcj),(Xag,Ce, YZck),(Xag,Ce,YZcl),(Xag,Ce,YZcm),(Xag,Ce,YZcn),(Xag,Ce,YZco),(Xag,Ce,YZcp), (Xag,Ce,YZcq),(Xag,Ce,YZcr),(Xag,Ce,YZcs),(Xag,Ce,YZct),(Xag,Ce,YZcu),(Xag, Ce,YZcv),(Xag,Ce,YZcw),(Xag,Ce,YZcx),(Xag,Ce,YZcy),(Xag,Ce,YZcz),(Xag,Ce,YZda), (Xag,Ce,YZdb),(Xag,Ce,YZdc),(Xag,Ce,YZdd),(Xag,Ce,YZde),(Xag,Ce,YZdf),(Xag, Ce,YZdg),(Xag,Ce,YZdh),(Xag,Ce,YZdi),(Xag,Ce,YZdj),(Xag,Ce,YZdk),(Xag,Ce, YZdl),(Xag,Ce,YZdm),(Xag,Ce,YZdn),(Xag,Ce,YZdo),(Xag,Ce,YZdp),(Xag,Ce,YZdq), (Xag,Ce,YZdr),(Xag,Ce,YZds),(Xag,Ce,Yzdt),(Xag,Ce,YZdu),(Xag,Ce,YZdv),(Xag, Ce,YZdw),(Xag,Ce,YZdx),(Xag,Ce,YZdy),(Xag,Ce,YZdz),(Xag,Ce,YZea),(Xag,Ce, YZeb),(Xag,Ce,YZec),(Xag,Ce,YZed),(Xag,Ce,YZee),(Xag,Ce,YZef),(Xag,Ce,YZeg), (Xag,Ce,YZeh),(Xag,Ce,YZei),(Xag,Ce,YZej),(Xag,Ce,YZek),(Xag,Ce,YZel),(Xag,Ce, YZem),(Xag,Ce,YZen),(Xag,Ce,YZeo),(Xag,Ce,YZep),(Xag,Ce,YZeq),(Xag,Ce,YZer), (Xag,Ce,YZes),(Xag,Ce,YZet),(Xag,Ce,YZeu),(Xag,Ce,YZev),(Xag,Ce,YZew),(Xag, Ce,YZex),(Xag,Ce,YZey),(Xag,Ce,YZez),(Xag,Ce,YZfa),(Xag,Ce,YZfb),(Xag,Ce, YZfc),(Xag,Ce,YZfd),(Xag,Ce,YZfe),(Xag,Ce,YZff),(Xag,Ce,YZfg),(Xag,Ce,YZfh),(Xag, Ce,YZfi),(Xag,Ce,YZfj),(Xag,Ce,YZfk),(Xag,Ce,YZfl),(Xag,Ce,YZfm),(Xag,Ce, YZfn),(Xag,Ce,YZfo),(Xag,Ce,YZfp),(Xag,Ce,YZfq),(Xag,Ce,YZfr),(Xag,Ce,YZfs),(Xag, Ce,YZft),(Xag,Ce,YZfu),(Xag,Ce,YZfv),(Xag,Ce,YZfw),(Xag,Ce,YZfx),(Xag,Ce, YZfy),(Xag,Ce,YZfz),(Xag,Ce,YZga),(Xag,Ce,YZgb),(Xag,Ce,YZgc),(Xag,Ce,YZgd), (Xag,Ce,YZge),(Xag,Ce,YZgf),(Xag,Ce,YZgg),(Xag,Ce,YZgh),(Xag,Ce,YZgi),(Xag, Ce,YZgj),(Xag,Ce,YZgk),(Xag,Ce,YZgl),(Xag,Ce,YZgm),(Xag,Ce,YZgn),(Xag,Ce,YZgo), (Xag,Ce,YZgp),(Xag,Ce,YZgq),(Xag,Ce,YZgr),(Xag,Ce,YZgs),(Xag,Ce,YZgt),(Xag, Cf,YZa),(Xag,Cf,YZb),(Xag,Cf,YZc),(Xag,Cf,YZd),(Xag,Cf,YZe),(Xag,Cf,YZf), (Xag,Cf,YZg),(Xag,Cf,YZh),(Xag,Cf,YZi),(Xag,Cf,YZj),(Xag,Cf,YZk),(Xag,Cf,YZl), (Xag,Cf,YZm),(Xag,Cf,YZn),(Xag,Cf,YZo),(Xag,Cf,YZp),(Xag,Cf,YZq),(Xag,Cf,YZr), (Xag,Cf,YZs),(Xag,Cf,YZt),(Xag,Cf,YZu),(Xag,Cf,YZv),(Xag,Cf,YZw),(Xag,Cf,YZx), (Xag,Cf,YZy),(Xag,Cf,YZz),(Xag,Cf,YZaa),(Xag,Cf,YZab),(Xag,Cf,YZac),(Xag, Cf,YZad),(Xag,Cf,YZae),(Xag,Cf,YZaf),(Xag,Cf,YZag),(Xag,Cf,YZah),(Xag,Cf,YZai), (Xag,Cf,YZaj),(Xag,Cf,YZak),(Xag,Cf,YZal),(Xag,Cf,YZam),(Xag,Cf,YZan),(Xag, Cf,YZao),(Xag,Cf,YZap),(Xag,Cf,YZaq),(Xag,Cf,YZar),(Xag,Cf,YZas),(Xag,Cf,YZat), (Xag,Cf,YZau),(Xag,Cf,YZav),(Xag,Cf,YZaw),(Xag,Cf,YZax),(Xag,Cf,YZay),(Xag, Cf,YZaz),(Xag,Cf,YZba),(Xag,Cf,YZbb),(Xag,Cf,YZbc),(Xag,Cf,YZbd),(Xag,Cf,YZbe), (Xag,Cf,YZbf),(Xag,Cf,YZbg),(Xag,Cf,YZbh),(Xag,Cf,YZbi),(Xag,Cf,YZbj),(Xag, Cf,YZbk),(Xag,Cf,YZbl),(Xag,Cf,YZbm),(Xag,Cf,YZbn),(Xag,Cf,YZbo),(Xag,Cf,YZbp), (Xag,Cf,YZbq),(Xag,Cf,YZbr),(Xag,Cf,YZbs),(Xag,Cf,YZbt),(Xag,Cf,YZbu),(Xag, Cf,YZbv),(Xag,Cf,YZbw),(Xag,Cf,YZbx),(Xag,Cf,YZby),(Xag,Cf,YZbz),(Xag,Cf,YZca), (Xag,Cf,YZcb),(Xag,Cf,YZcc),(Xag,Cf,YZcd),(Xag,Cf,YZce),(Xag,Cf,YZcf),(Xag, Cf,YZcg),(Xag,Cf,YZch),(Xag,Cf,YZci),(Xag,Cf,YZcj),(Xag,Cf,YZck),(Xag,Cf,YZcl), (Xag,Cf,YZcm),(Xag,Cf,YZcn),(Xag,Cf,YZco),(Xag,Cf,YZcp),(Xag,Cf,YZcq),(Xag, Cf,YZcr),(Xag,Cf,YZcs),(Xag,Cf,YZct),(Xag,Cf,YZcu),(Xag,Cf,YZcv),(Xag,Cf, YZcw),(Xag,Cf,YZcx),(Xag,Cf,YZcy),(Xag,Cf,YZcz),(Xag,Cf,YZda),(Xag,Cf,YZdb), (Xag,Cf,YZdc),(Xag,Cf,YZdd),(Xag,Cf,YZde),(Xag,Cf,YZdf),(Xag,Cf,YZdg),(Xag,Cf, YZdh),(Xag,Cf,YZdi),(Xag,Cf,YZdj),(Xag,Cf,YZdk),(Xag,Cf,YZdl),(Xag,Cf,YZdm), (Xag,Cf,YZdn),(Xag,Cf,YZdo),(Xag,Cf,YZdp),(Xag,Cf,YZdq),(Xag,Cf,YZdr),(Xag, Cf,YZds),(Xag,Cf,Yzdt),(Xag,Cf,YZdu),(Xag,Cf,YZdv),(Xag,Cf,YZdw),(Xag,Cf,YZdx), (Xag,Cf,YZdy),(Xag,Cf,YZdz),(Xag,Cf,YZea),(Xag,Cf,YZeb),(Xag,Cf,YZec),(Xag, Cf,YZed),(Xag,Cf,YZee),(Xag,Cf,YZef),(Xag,Cf,YZeg),(Xag,Cf,YZeh),(Xag,Cf,YZei), (Xag,Cf,YZej),(Xag,Cf,YZek),(Xag,Cf,YZel),(Xag,Cf,YZem),(Xag,Cf,YZen),(Xag, Cf,YZeo),(Xag,Cf,YZep),(Xag,Cf,YZeq),(Xag,Cf,YZer),(Xag,Cf,YZes),(Xag,Cf,YZet), (Xag,Cf,YZeu),(Xag,Cf,YZev),(Xag,Cf,YZew),(Xag,Cf,YZex),(Xag,Cf,YZey),(Xag, Cf,YZez),(Xag,Cf,YZfa),(Xag,Cf,YZfb),(Xag,Cf,YZfc),(Xag,Cf,YZfd),(Xag,Cf,YZfe), (Xag,Cf,YZff),(Xag,Cf,YZfg),(Xag,Cf,YZfh),(Xag,Cf,YZfi),(Xag,Cf,YZfj),(Xag,Cf, YZfk),(Xag,Cf,YZfl),(Xag,Cf,YZfm),(Xag,Cf,YZfn),(Xag,Cf,YZfo),(Xag,Cf,YZfp),(Xag, Cf,YZfq),(Xag,Cf,YZfr),(Xag,Cf,YZfs),(Xag,Cf,YZft),(Xag,Cf,YZfu),(Xag,Cf,YZfv), (Xag,Cf,YZfw),(Xag,Cf,YZfx),(Xag,Cf,YZfy),(Xag,Cf,YZfz),(Xag,Cf,YZga),(Xag, Cf,YZgb),(Xag,Cf,YZgc),(Xag,Cf,YZgd),(Xag,Cf,YZge),(Xag,Cf,YZgf),(Xag,Cf,YZgg), (Xag,Cf,YZgh),(Xag,Cf,YZgi),(Xag,Cf,YZgj),(Xag,Cf,YZgk),(Xag,Cf,YZgl),(Xag, Cf,YZgm),(Xag,Cf,YZgn),(Xag,Cf,YZgo),(Xag,Cf,YZgp),(Xag,Cf,YZgq),(Xag,Cf, YZgr),(Xag,Cf,YZgs),(Xag,Cf,YZgt), (Xah,Ca,YZa),(Xah,Ca,YZb),(Xah,Ca,YZc),(Xah,Ca,YZd),(Xah,Ca,YZe),(Xah,Ca,YZf), (Xah,Ca,YZg),(Xah,Ca,YZh),(Xah,Ca,YZi),(Xah,Ca,YZj),(Xah,Ca,YZk),(Xah,Ca,YZl), (Xah,Ca,YZm),(Xah,Ca,YZn),(Xah,Ca,YZo),(Xah,Ca,YZp),(Xah,Ca,YZq),(Xah,Ca, YZr),(Xah,Ca,YZs),(Xah,Ca,YZt),(Xah,Ca,YZu),(Xah,Ca,YZv),(Xah,Ca,YZw),(Xah, Ca,YZx),(Xah,Ca,YZy),(Xah,Ca,YZz),(Xah,Ca,YZaa),(Xah,Ca,YZab),(Xah,Ca,YZac), (Xah,Ca,YZad),(Xah,Ca,YZae),(Xah,Ca,YZaf),(Xah,Ca,YZag),(Xah,Ca,YZah),(Xah, Ca,YZai),(Xah,Ca,YZaj),(Xah,Ca,YZak),(Xah,Ca,YZal),(Xah,Ca,YZam),(Xah,Ca,YZan), (Xah,Ca,YZao),(Xah,Ca,YZap),(Xah,Ca,YZaq),(Xah,Ca,YZar),(Xah,Ca,YZas),(Xah, Ca,YZat),(Xah,Ca,YZau),(Xah,Ca,YZav),(Xah,Ca,YZaw),(Xah,Ca,YZax),(Xah,Ca, YZay),(Xah,Ca,YZaz),(Xah,Ca,YZba),(Xah,Ca,YZbb),(Xah,Ca,YZbc),(Xah,Ca,YZbd), (Xah,Ca,YZbe),(Xah,Ca,YZbf),(Xah,Ca,YZbg),(Xah,Ca,YZbh),(Xah,Ca,YZbi),(Xah, Ca,YZbj),(Xah,Ca,YZbk),(Xah,Ca,YZbl),(Xah,Ca,YZbm),(Xah,Ca,YZbn),(Xah,Ca,YZbo), (Xah,Ca,YZbp),(Xah,Ca,YZbq),(Xah,Ca,YZbr),(Xah,Ca,YZbs),(Xah,Ca,YZbt),(Xah, Ca,YZbu),(Xah,Ca,YZbv),(Xah,Ca,YZbw),(Xah,Ca,YZbx),(Xah,Ca,YZby),(Xah, Ca,YZbz),(Xah,Ca,YZca),(Xah,Ca,YZcb),(Xah,Ca,YZcc),(Xah,Ca,YZcd),(Xah,Ca,YZce), (Xah,Ca,YZcf),(Xah,Ca,YZcg),(Xah,Ca,YZch),(Xah,Ca,YZci),(Xah,Ca,YZcj),(Xah, Ca,YZck),(Xah,Ca,YZcl),(Xah,Ca,YZcm),(Xah,Ca,YZcn),(Xah,Ca,YZco),(Xah,Ca, YZcp),(Xah,Ca,YZcq),(Xah,Ca,YZcr),(Xah,Ca,YZcs),(Xah,Ca,YZct),(Xah,Ca,YZcu), (Xah,Ca,YZcv),(Xah,Ca,YZcw),(Xah,Ca,YZcx),(Xah,Ca,YZcy),(Xah,Ca,YZcz),(Xah, Ca,YZda),(Xah,Ca,YZdb),(Xah,Ca,YZdc),(Xah,Ca,YZdd),(Xah,Ca,YZde),(Xah,Ca,YZdf), (Xah,Ca,YZdg),(Xah,Ca,YZdh),(Xah,Ca,YZdi),(Xah,Ca,YZdj),(Xah,Ca,YZdk),(Xah, Ca,YZdl),(Xah,Ca,YZdm),(Xah,Ca,YZdn),(Xah,Ca,YZdo),(Xah,Ca,YZdp),(Xah, Ca,YZdq),(Xah,Ca,YZdr),(Xah,Ca,YZds),(Xah,Ca,Yzdt),(Xah,Ca,YZdu),(Xah,Ca,YZdv), (Xah,Ca,YZdw),(Xah,Ca,YZdx),(Xah,Ca,YZdy),(Xah,Ca,YZdz),(Xah,Ca,YZea),(Xah, Ca,YZeb),(Xah,Ca,YZec),(Xah,Ca,YZed),(Xah,Ca,YZee),(Xah,Ca,YZef),(Xah,Ca, YZeg),(Xah,Ca,YZeh),(Xah,Ca,YZei),(Xah,Ca,YZej),(Xah,Ca,YZek),(Xah,Ca,YZel), (Xah,Ca,YZem),(Xah,Ca,YZen),(Xah,Ca,YZeo),(Xah,Ca,YZep),(Xah,Ca,YZeq),(Xah, Ca,YZer),(Xah,Ca,YZes),(Xah,Ca,YZet),(Xah,Ca,YZeu),(Xah,Ca,YZev),(Xah,Ca,YZew), (Xah,Ca,YZex),(Xah,Ca,YZey),(Xah,Ca,YZez),(Xah,Ca,YZfa),(Xah,Ca,YZfb),(Xah, Ca,YZfc),(Xah,Ca,YZfd),(Xah,Ca,YZfe),(Xah,Ca,YZff),(Xah,Ca,YZfg),(Xah,Ca,YZfh), (Xah,Ca,YZfi),(Xah,Ca,YZfj),(Xah,Ca,YZfk),(Xah,Ca,YZfl),(Xah,Ca,YZfm),(Xah, Ca,YZfn),(Xah,Ca,YZfo),(Xah,Ca,YZfp),(Xah,Ca,YZfq),(Xah,Ca,YZfr),(Xah,Ca,YZfs), (Xah,Ca,YZft),(Xah,Ca,YZfu),(Xah,Ca,YZfv),(Xah,Ca,YZfw),(Xah,Ca,YZfx),(Xah, Ca,YZfy),(Xah,Ca,YZfz),(Xah,Ca,YZga),(Xah,Ca,YZgb),(Xah,Ca,YZgc),(Xah,Ca, YZgd),(Xah,Ca,YZge),(Xah,Ca,YZgf),(Xah,Ca,YZgg),(Xah,Ca,YZgh),(Xah,Ca,YZgi), (Xah,Ca,YZgj),(Xah,Ca,YZgk),(Xah,Ca,YZgl),(Xah,Ca,YZgm),(Xah,Ca,YZgn),(Xah, Ca,YZgo),(Xah,Ca,YZgp),(Xah,Ca,YZgq),(Xah,Ca,YZgr),(Xah,Ca,YZgs),(Xah,Ca,YZgt), (Xah,Cb,YZa),(Xah,Cb,YZb),(Xah,Cb,YZc),(Xah,Cb,YZd),(Xah,Cb,YZe),(Xah, Cb,YZf),(Xah,Cb,YZg),(Xah,Cb,YZh),(Xah,Cb,YZi),(Xah,Cb,YZj),(Xah,Cb,YZk),(Xah, Cb,YZl),(Xah,Cb,YZm),(Xah,Cb,YZn),(Xah,Cb,YZo),(Xah,Cb,YZp),(Xah,Cb,YZq), (Xah,Cb,YZr),(Xah,Cb,YZs),(Xah,Cb,YZt),(Xah,Cb,YZu),(Xah,Cb,YZv),(Xah,Cb,YZw), (Xah,Cb,YZx),(Xah,Cb,YZy),(Xah,Cb,YZz),(Xah,Cb,YZaa),(Xah,Cb,YZab),(Xah, Cb,YZac),(Xah,Cb,YZad),(Xah,Cb,YZae),(Xah,Cb,YZaf),(Xah,Cb,YZag),(Xah,Cb, YZah),(Xah,Cb,YZai),(Xah,Cb,YZaj),(Xah,Cb,YZak),(Xah,Cb,YZal),(Xah,Cb,YZam), (Xah,Cb,YZan),(Xah,Cb,YZao),(Xah,Cb,YZap),(Xah,Cb,YZaq),(Xah,Cb,YZar),(Xah, Cb,YZas),(Xah,Cb,YZat),(Xah,Cb,YZau),(Xah,Cb,YZav),(Xah,Cb,YZaw),(Xah,Cb,YZax), (Xah,Cb,YZay),(Xah,Cb,YZaz),(Xah,Cb,YZba),(Xah,Cb,YZbb),(Xah,Cb,YZbc), (Xah,Cb,YZbd),(Xah,Cb,YZbe),(Xah,Cb,YZbf),(Xah,Cb,YZbg),(Xah,Cb,YZbh),(Xah, Cb,YZbi),(Xah,Cb,YZbj),(Xah,Cb,YZbk),(Xah,Cb,YZbl),(Xah,Cb,YZbm),(Xah,Cb,YZbn), (Xah,Cb,YZbo),(Xah,Cb,YZbp),(Xah,Cb,YZbq),(Xah,Cb,YZbr),(Xah,Cb,YZbs), (Xah,Cb,YZbt),(Xah,Cb,YZbu),(Xah,Cb,YZbv),(Xah,Cb,YZbw),(Xah,Cb,YZbx),(Xah, Cb,YZby),(Xah,Cb,YZbz),(Xah,Cb,YZca),(Xah,Cb,YZcb),(Xah,Cb,YZcc),(Xah,Cb,YZcd), (Xah,Cb,YZce),(Xah,Cb,YZcf),(Xah,Cb,YZcg),(Xah,Cb,YZch),(Xah,Cb,YZci),(Xah, Cb,YZcj),(Xah,Cb,YZck),(Xah,Cb,YZcl),(Xah,Cb,YZcm),(Xah,Cb,YZcn),(Xah, Cb,YZco),(Xah,Cb,YZcp),(Xah,Cb,YZcq),(Xah,Cb,YZcr),(Xah,Cb,YZcs),(Xah,Cb,YZct), (Xah,Cb,YZcu),(Xah,Cb,YZcv),(Xah,Cb,YZcw),(Xah,Cb,YZcx),(Xah,Cb,YZcy), (Xah,Cb,YZcz),(Xah,Cb,YZda),(Xah,Cb,YZdb),(Xah,Cb,YZdc),(Xah,Cb,YZdd),(Xah, Cb,YZde),(Xah,Cb,YZdf),(Xah,Cb,YZdg),(Xah,Cb,YZdh),(Xah,Cb,YZdi),(Xah,Cb,YZdj), (Xah,Cb,YZdk),(Xah,Cb,YZdl),(Xah,Cb,YZdm),(Xah,Cb,YZdn),(Xah,Cb,YZdo), (Xah,Cb,YZdp),(Xah,Cb,YZdq),(Xah,Cb,YZdr),(Xah,Cb,YZds),(Xah,Cb,Yzdt),(Xah, Cb,YZdu),(Xah,Cb,YZdv),(Xah,Cb,YZdw),(Xah,Cb,YZdx),(Xah,Cb,YZdy),(Xah,Cb, YZdz),(Xah,Cb,YZea),(Xah,Cb,YZeb),(Xah,Cb,YZec),(Xah,Cb,YZed),(Xah,Cb,YZee), (Xah,Cb,YZef),(Xah,Cb,YZeg),(Xah,Cb,YZeh),(Xah,Cb,YZei),(Xah,Cb,YZej),(Xah, Cb,YZek),(Xah,Cb,YZel),(Xah,Cb,YZem),(Xah,Cb,YZen),(Xah,Cb,YZeo),(Xah,Cb,YZep), (Xah,Cb,YZeq),(Xah,Cb,YZer),(Xah,Cb,YZes),(Xah,Cb,YZet),(Xah,Cb,YZeu),(Xah, Cb,YZev),(Xah,Cb,YZew),(Xah,Cb,YZex),(Xah,Cb,YZey),(Xah,Cb,YZez),(Xah, Cb,YZfa),(Xah,Cb,YZfb),(Xah,Cb,YZfc),(Xah,Cb,YZfd),(Xah,Cb,YZfe),(Xah,Cb,YZff), (Xah,Cb,YZfg),(Xah,Cb,YZfh),(Xah,Cb,YZfi),(Xah,Cb,YZfj),(Xah,Cb,YZfk),(Xah, Cb,YZfl),(Xah,Cb,YZfm),(Xah,Cb,YZfn),(Xah,Cb,YZfo),(Xah,Cb,YZfp),(Xah,Cb,YZfq), (Xah,Cb,YZfr),(Xah,Cb,YZfs),(Xah,Cb,YZft),(Xah,Cb,YZfu),(Xah,Cb,YZfv),(Xah, Cb,YZfw),(Xah,Cb,YZfx),(Xah,Cb,YZfy),(Xah,Cb,YZfz),(Xah,Cb,YZga),(Xah,Cb,YZgb), (Xah,Cb,YZgc),(Xah,Cb,YZgd),(Xah,Cb,YZge),(Xah,Cb,YZgf),(Xah,Cb,YZgg), (Xah,Cb,YZgh),(Xah,Cb,YZgi),(Xah,Cb,YZgj),(Xah,Cb,YZgk),(Xah,Cb,YZgl),(Xah, Cb,YZgm),(Xah,Cb,YZgn),(Xah,Cb,YZgo),(Xah,Cb,YZgp),(Xah,Cb,YZgq),(Xah,Cb, YZgr),(Xah,Cb,YZgs),(Xah,Cb,YZgt),(Xah,Cc,YZa),(Xah,Cc,YZb),(Xah,Cc,YZc),(Xah, Cc,YZd),(Xah,Cc,YZe),(Xah,Cc,YZf),(Xah,Cc,YZg),(Xah,Cc,YZh),(Xah,Cc,YZi),(Xah, Cc,YZj),(Xah,Cc,YZk),(Xah,Cc,YZl),(Xah,Cc,YZm),(Xah,Cc,YZn),(Xah,Cc,YZo), (Xah,Cc,YZp),(Xah,Cc,YZq),(Xah,Cc,YZr),(Xah,Cc,YZs),(Xah,Cc,YZt),(Xah,Cc, YZu),(Xah,Cc,YZv),(Xah,Cc,YZw),(Xah,Cc,YZx),(Xah,Cc,YZy),(Xah,Cc,YZz),(Xah, Cc,YZaa),(Xah,Cc,YZab),(Xah,Cc,YZac),(Xah,Cc,YZad),(Xah,Cc,YZae),(Xah,Cc,YZaf), (Xah,Cc,YZag),(Xah,Cc,YZah),(Xah,Cc,YZai),(Xah,Cc,YZaj),(Xah,Cc,YZak),(Xah, Cc,YZal),(Xah,Cc,YZam),(Xah,Cc,YZan),(Xah,Cc,YZao),(Xah,Cc,YZap),(Xah,Cc, YZaq),(Xah,Cc,YZar),(Xah,Cc,YZas),(Xah,Cc,YZat),(Xah,Cc,YZau),(Xah,Cc,YZav), (Xah,Cc,YZaw),(Xah,Cc,YZax),(Xah,Cc,YZay),(Xah,Cc,YZaz),(Xah,Cc,YZba),(Xah, Cc,YZbb),(Xah,Cc,YZbc),(Xah,Cc,YZbd),(Xah,Cc,YZbe),(Xah,Cc,YZbf),(Xah,Cc,YZbg), (Xah,Cc,YZbh),(Xah,Cc,YZbi),(Xah,Cc,YZbj),(Xah,Cc,YZbk),(Xah,Cc,YZbl),(Xah, Cc,YZbm),(Xah,Cc,YZbn),(Xah,Cc,YZbo),(Xah,Cc,YZbp),(Xah,Cc,YZbq),(Xah, Cc,YZbr),(Xah,Cc,YZbs),(Xah,Cc,YZbt),(Xah,Cc,YZbu),(Xah,Cc,YZbv),(Xah,Cc,YZbw), (Xah,Cc,YZbx),(Xah,Cc,YZby),(Xah,Cc,YZbz),(Xah,Cc,YZca),(Xah,Cc,YZcb),(Xah, Cc,YZcc),(Xah,Cc,YZcd),(Xah,Cc,YZce),(Xah,Cc,YZcf),(Xah,Cc,YZcg),(Xah,Cc, YZch),(Xah,Cc,YZci),(Xah,Cc,YZcj),(Xah,Cc,YZck),(Xah,Cc,YZcl),(Xah,Cc,YZcm), (Xah,Cc,YZcn),(Xah,Cc,YZco),(Xah,Cc,YZcp),(Xah,Cc,YZcq),(Xah,Cc,YZcr),(Xah, Cc,YZcs),(Xah,Cc,YZct),(Xah,Cc,YZcu),(Xah,Cc,YZcv),(Xah,Cc,YZcw),(Xah,Cc,YZcx), (Xah,Cc,YZcy),(Xah,Cc,YZcz),(Xah,Cc,YZda),(Xah,Cc,YZdb),(Xah,Cc,YZdc),(Xah, Cc,YZdd),(Xah,Cc,YZde),(Xah,Cc,YZdf),(Xah,Cc,YZdg),(Xah,Cc,YZdh),(Xah,Cc, YZdi),(Xah,Cc,YZdj),(Xah,Cc,YZdk),(Xah,Cc,YZdl),(Xah,Cc,YZdm),(Xah,Cc,YZdn), (Xah,Cc,YZdo),(Xah,Cc,YZdp),(Xah,Cc,YZdq),(Xah,Cc,YZdr),(Xah,Cc,YZds),(Xah, Cc,Yzdt),(Xah,Cc,YZdu),(Xah,Cc,YZdv),(Xah,Cc,YZdw),(Xah,Cc,YZdx),(Xah,Cc, YZdy),(Xah,Cc,YZdz),(Xah,Cc,YZea),(Xah,Cc,YZeb),(Xah,Cc,YZec),(Xah,Cc,YZed), (Xah,Cc,YZee),(Xah,Cc,YZef),(Xah,Cc,YZeg),(Xah,Cc,YZeh),(Xah,Cc,YZei),(Xah, Cc,YZej),(Xah,Cc,YZek),(Xah,Cc,YZel),(Xah,Cc,YZem),(Xah,Cc,YZen),(Xah,Cc,YZeo), (Xah,Cc,YZep),(Xah,Cc,YZeq),(Xah,Cc,YZer),(Xah,Cc,YZes),(Xah,Cc,YZet),(Xah, Cc,YZeu),(Xah,Cc,YZev),(Xah,Cc,YZew),(Xah,Cc,YZex),(Xah,Cc,YZey),(Xah,Cc, YZez),(Xah,Cc,YZfa),(Xah,Cc,YZfb),(Xah,Cc,YZfc),(Xah,Cc,YZfd),(Xah,Cc,YZfe),(Xah, Cc,YZff),(Xah,Cc,YZfg),(Xah,Cc,YZfh),(Xah,Cc,YZfi),(Xah,Cc,YZfj),(Xah,Cc, YZfk),(Xah,Cc,YZfl),(Xah,Cc,YZfm),(Xah,Cc,YZfn),(Xah,Cc,YZfo),(Xah,Cc,YZfp),(Xah, Cc,YZfq),(Xah,Cc,YZfr),(Xah,Cc,YZfs),(Xah,Cc,YZft),(Xah,Cc,YZfu),(Xah,Cc, YZfv),(Xah,Cc,YZfw),(Xah,Cc,YZfx),(Xah,Cc,YZfy),(Xah,Cc,YZfz),(Xah,Cc,YZga), (Xah,Cc,YZgb),(Xah,Cc,YZgc),(Xah,Cc,YZgd),(Xah,Cc,YZge),(Xah,Cc,YZgf),(Xah, Cc,YZgg),(Xah,Cc,YZgh),(Xah,Cc,YZgi),(Xah,Cc,YZgj),(Xah,Cc,YZgk),(Xah,Cc,YZgl), (Xah,Cc,YZgm),(Xah,Cc,YZgn),(Xah,Cc,YZgo),(Xah,Cc,YZgp),(Xah,Cc,YZgq),(Xah, Cc,YZgr),(Xah,Cc,YZgs),(Xah,Cc,YZgt),(Xah,Cd,YZa),(Xah,Cd,YZb),(Xah,Cd, YZc),(Xah,Cd,YZd),(Xah,Cd,YZe),(Xah,Cd,YZf),(Xah,Cd,YZg),(Xah,Cd,YZh),(Xah, Cd,YZi),(Xah,Cd,YZj),(Xah,Cd,YZk),(Xah,Cd,YZl),(Xah,Cd,YZm),(Xah,Cd,YZn),(Xah, Cd,YZo),(Xah,Cd,YZp),(Xah,Cd,YZq),(Xah,Cd,YZr),(Xah,Cd,YZs),(Xah,Cd,YZt), (Xah,Cd,YZu),(Xah,Cd,YZv),(Xah,Cd,YZw),(Xah,Cd,YZx),(Xah,Cd,YZy),(Xah,Cd, YZz),(Xah,Cd,YZaa),(Xah,Cd,YZab),(Xah,Cd,YZac),(Xah,Cd,YZad),(Xah,Cd,YZae), (Xah,Cd,YZaf),(Xah,Cd,YZag),(Xah,Cd,YZah),(Xah,Cd,YZai),(Xah,Cd,YZaj),(Xah,Cd, YZak),(Xah,Cd,YZal),(Xah,Cd,YZam),(Xah,Cd,YZan),(Xah,Cd,YZao),(Xah,Cd,YZap), (Xah,Cd,YZaq),(Xah,Cd,YZar),(Xah,Cd,YZas),(Xah,Cd,YZat),(Xah,Cd,YZau),(Xah, Cd,YZav),(Xah,Cd,YZaw),(Xah,Cd,YZax),(Xah,Cd,YZay),(Xah,Cd,YZaz),(Xah,Cd, YZba),(Xah,Cd,YZbb),(Xah,Cd,YZbc),(Xah,Cd,YZbd),(Xah,Cd,YZbe),(Xah,Cd,YZbf), (Xah,Cd,YZbg),(Xah,Cd,YZbh),(Xah,Cd,YZbi),(Xah,Cd,YZbj),(Xah,Cd,YZbk),(Xah, Cd,YZbl),(Xah,Cd,YZbm),(Xah,Cd,YZbn),(Xah,Cd,YZbo),(Xah,Cd,YZbp),(Xah,Cd, YZbq),(Xah,Cd,YZbr),(Xah,Cd,YZbs),(Xah,Cd,YZbt),(Xah,Cd,YZbu),(Xah,Cd,YZbv), (Xah,Cd,YZbw),(Xah,Cd,YZbx),(Xah,Cd,YZby),(Xah,Cd,YZbz),(Xah,Cd,YZca),(Xah, Cd,YZcb),(Xah,Cd,YZcc),(Xah,Cd,YZcd),(Xah,Cd,YZce),(Xah,Cd,YZcf),(Xah,Cd, YZcg),(Xah,Cd,YZch),(Xah,Cd,YZci),(Xah,Cd,YZcj),(Xah,Cd,YZck),(Xah,Cd,YZcl), (Xah,Cd,YZcm),(Xah,Cd,YZcn),(Xah,Cd,YZco),(Xah,Cd,YZcp),(Xah,Cd,YZcq),(Xah, Cd,YZcr),(Xah,Cd,YZcs),(Xah,Cd,YZct),(Xah,Cd,YZcu),(Xah,Cd,YZcv),(Xah,Cd, YZcw),(Xah,Cd,YZcx),(Xah,Cd,YZcy),(Xah,Cd,YZcz),(Xah,Cd,YZda),(Xah,Cd,YZdb), (Xah,Cd,YZdc),(Xah,Cd,YZdd),(Xah,Cd,YZde),(Xah,Cd,YZdf),(Xah,Cd,YZdg),(Xah, Cd,YZdh),(Xah,Cd,YZdi),(Xah,Cd,YZdj),(Xah,Cd,YZdk),(Xah,Cd,YZdl),(Xah,Cd, YZdm),(Xah,Cd,YZdn),(Xah,Cd,YZdo),(Xah,Cd,YZdp),(Xah,Cd,YZdq),(Xah,Cd,YZdr), (Xah,Cd,YZds),(Xah,Cd,Yzdt),(Xah,Cd,YZdu),(Xah,Cd,YZdv),(Xah,Cd,YZdw),(Xah, Cd,YZdx),(Xah,Cd,YZdy),(Xah,Cd,YZdz),(Xah,Cd,YZea),(Xah,Cd,YZeb),(Xah, Cd,YZec),(Xah,Cd,YZed),(Xah,Cd,YZee),(Xah,Cd,YZef),(Xah,Cd,YZeg),(Xah,Cd,YZeh), (Xah,Cd,YZei),(Xah,Cd,YZej),(Xah,Cd,YZek),(Xah,Cd,YZel),(Xah,Cd,YZem),(Xah, Cd,YZen),(Xah,Cd,YZeo),(Xah,Cd,YZep),(Xah,Cd,YZeq),(Xah,Cd,YZer),(Xah, Cd,YZes),(Xah,Cd,YZet),(Xah,Cd,YZeu),(Xah,Cd,YZev),(Xah,Cd,YZew),(Xah,Cd,YZex), (Xah,Cd,YZey),(Xah,Cd,YZez),(Xah,Cd,YZfa),(Xah,Cd,YZfb),(Xah,Cd,YZfc),(Xah, Cd,YZfd),(Xah,Cd,YZfe),(Xah,Cd,YZff),(Xah,Cd,YZfg),(Xah,Cd,YZfh),(Xah,Cd, YZfi),(Xah,Cd,YZfj),(Xah,Cd,YZfk),(Xah,Cd,YZfl),(Xah,Cd,YZfm),(Xah,Cd,YZfn), (Xah,Cd,YZfo),(Xah,Cd,YZfp),(Xah,Cd,YZfq),(Xah,Cd,YZfr),(Xah,Cd,YZfs),(Xah,Cd, YZft),(Xah,Cd,YZfu),(Xah,Cd,YZfv),(Xah,Cd,YZfw),(Xah,Cd,YZfx),(Xah,Cd,YZfy), (Xah,Cd,YZfz),(Xah,Cd,YZga),(Xah,Cd,YZgb),(Xah,Cd,YZgc),(Xah,Cd,YZgd),(Xah, Cd,YZge),(Xah,Cd,YZgf),(Xah,Cd,YZgg),(Xah,Cd,YZgh),(Xah,Cd,YZgi),(Xah,Cd, YZgj),(Xah,Cd,YZgk),(Xah,Cd,YZgl),(Xah,Cd,YZgm),(Xah,Cd,YZgn),(Xah,Cd,YZgo), (Xah,Cd,YZgp),(Xah,Cd,YZgq),(Xah,Cd,YZgr),(Xah,Cd,YZgs),(Xah,Cd,YZgt),(Xah, Ce,YZa),(Xah,Ce,YZb),(Xah,Ce,YZc),(Xah,Ce,YZd),(Xah,Ce,YZe),(Xah,Ce,YZf),(Xah, Ce,YZg),(Xah,Ce,YZh),(Xah,Ce,YZi),(Xah,Ce,YZj),(Xah,Ce,YZk),(Xah,Ce,YZl), (Xah,Ce,YZm),(Xah,Ce,YZn),(Xah,Ce,YZo),(Xah,Ce,YZp),(Xah,Ce,YZq),(Xah,Ce,YZr), (Xah,Ce,YZs),(Xah,Ce,YZt),(Xah,Ce,YZu),(Xah,Ce,YZv),(Xah,Ce,YZw),(Xah,Ce, YZx),(Xah,Ce,YZy),(Xah,Ce,YZz),(Xah,Ce,YZaa),(Xah,Ce,YZab),(Xah,Ce,YZac),(Xah, Ce,YZad),(Xah,Ce,YZae),(Xah,Ce,YZaf),(Xah,Ce,YZag),(Xah,Ce,YZah),(Xah,Ce, YZai),(Xah,Ce,YZaj),(Xah,Ce,YZak),(Xah,Ce,YZal),(Xah,Ce,YZam),(Xah,Ce,YZan), (Xah,Ce,YZao),(Xah,Ce,YZap),(Xah,Ce,YZaq),(Xah,Ce,YZar),(Xah,Ce,YZas),(Xah, Ce,YZat),(Xah,Ce,YZau),(Xah,Ce,YZav),(Xah,Ce,YZaw),(Xah,Ce,YZax),(Xah,Ce,YZay), (Xah,Ce,YZaz),(Xah,Ce,YZba),(Xah,Ce,YZbb),(Xah,Ce,YZbc),(Xah,Ce,YZbd),(Xah, Ce,YZbe),(Xah,Ce,YZbf),(Xah,Ce,YZbg),(Xah,Ce,YZbh),(Xah,Ce,YZbi),(Xah,Ce, YZbj),(Xah,Ce,YZbk),(Xah,Ce,YZbl),(Xah,Ce,YZbm),(Xah,Ce,YZbn),(Xah,Ce,YZbo), (Xah,Ce,YZbp),(Xah,Ce,YZbq),(Xah,Ce,YZbr),(Xah,Ce,YZbs),(Xah,Ce,YZbt),(Xah, Ce,YZbu),(Xah,Ce,YZbv),(Xah,Ce,YZbw),(Xah,Ce,YZbx),(Xah,Ce,YZby),(Xah,Ce, YZbz),(Xah,Ce,YZca),(Xah,Ce,YZcb),(Xah,Ce,YZcc),(Xah,Ce,YZcd),(Xah,Ce,YZce), (Xah,Ce,YZcf),(Xah,Ce,YZcg),(Xah,Ce,YZch),(Xah,Ce,YZci),(Xah,Ce,YZcj),(Xah,Ce, YZck),(Xah,Ce,YZcl),(Xah,Ce,YZcm),(Xah,Ce,YZcn),(Xah,Ce,YZco),(Xah,Ce,YZcp), (Xah,Ce,YZcq),(Xah,Ce,YZcr),(Xah,Ce,YZcs),(Xah,Ce,YZct),(Xah,Ce,YZcu),(Xah, Ce,YZcv),(Xah,Ce,YZcw),(Xah,Ce,YZcx),(Xah,Ce,YZcy),(Xah,Ce,YZcz),(Xah,Ce,YZda), (Xah,Ce,YZdb),(Xah,Ce,YZdc),(Xah,Ce,YZdd),(Xah,Ce,YZde),(Xah,Ce,YZdf),(Xah, Ce,YZdg),(Xah,Ce,YZdh),(Xah,Ce,YZdi),(Xah,Ce,YZdj),(Xah,Ce,YZdk),(Xah,Ce, YZdl),(Xah,Ce,YZdm),(Xah,Ce,YZdn),(Xah,Ce,YZdo),(Xah,Ce,YZdp),(Xah,Ce,YZdq), (Xah,Ce,YZdr),(Xah,Ce,YZds),(Xah,Ce,Yzdt),(Xah,Ce,YZdu),(Xah,Ce,YZdv),(Xah, Ce,YZdw),(Xah,Ce,YZdx),(Xah,Ce,YZdy),(Xah,Ce,YZdz),(Xah,Ce,YZea),(Xah,Ce, YZeb),(Xah,Ce,YZec),(Xah,Ce,YZed),(Xah,Ce,YZee),(Xah,Ce,YZef),(Xah,Ce,YZeg), (Xah,Ce,YZeh),(Xah,Ce,YZei),(Xah,Ce,YZej),(Xah,Ce,YZek),(Xah,Ce,YZel),(Xah,Ce, YZem),(Xah,Ce,YZen),(Xah,Ce,YZeo),(Xah,Ce,YZep),(Xah,Ce,YZeq),(Xah,Ce,YZer), (Xah,Ce,YZes),(Xah,Ce,YZet),(Xah,Ce,YZeu),(Xah,Ce,YZev),(Xah,Ce,YZew),(Xah, Ce,YZex),(Xah,Ce,YZey),(Xah,Ce,YZez),(Xah,Ce,YZfa),(Xah,Ce,YZfb),(Xah,Ce, YZfc),(Xah,Ce,YZfd),(Xah,Ce,YZfe),(Xah,Ce,YZff),(Xah,Ce,YZfg),(Xah,Ce,YZfh),(Xah, Ce,YZfi),(Xah,Ce,YZfj),(Xah,Ce,YZfk),(Xah,Ce,YZfl),(Xah,Ce,YZfm),(Xah,Ce, YZfn),(Xah,Ce,YZfo),(Xah,Ce,YZfp),(Xah,Ce,YZfq),(Xah,Ce,YZfr),(Xah,Ce,YZfs),(Xah, Ce,YZft),(Xah,Ce,YZfu),(Xah,Ce,YZfv),(Xah,Ce,YZfw),(Xah,Ce,YZfx),(Xah,Ce, YZfy),(Xah,Ce,YZfz),(Xah,Ce,YZga),(Xah,Ce,YZgb),(Xah,Ce,YZgc),(Xah,Ce,YZgd), (Xah,Ce,YZge),(Xah,Ce,YZgf),(Xah,Ce,YZgg),(Xah,Ce,YZgh),(Xah,Ce,YZgi),(Xah, Ce,YZgj),(Xah,Ce,YZgk),(Xah,Ce,YZgl),(Xah,Ce,YZgm),(Xah,Ce,YZgn),(Xah,Ce,YZgo), (Xah,Ce,YZgp),(Xah,Ce,YZgq),(Xah,Ce,YZgr),(Xah,Ce,YZgs),(Xah,Ce,YZgt),(Xah, Cf,YZa),(Xah,Cf,YZb),(Xah,Cf,YZc),(Xah,Cf,YZd),(Xah,Cf,YZe),(Xah,Cf,YZf), (Xah,Cf,YZg),(Xah,Cf,YZh),(Xah,Cf,YZi),(Xah,Cf,YZj),(Xah,Cf,YZk),(Xah,Cf,YZl), (Xah,Cf,YZm),(Xah,Cf,YZn),(Xah,Cf,YZo),(Xah,Cf,YZp),(Xah,Cf,YZq),(Xah,Cf,YZr), (Xah,Cf,YZs),(Xah,Cf,YZt),(Xah,Cf,YZu),(Xah,Cf,YZv),(Xah,Cf,YZw),(Xah,Cf,YZx), (Xah,Cf,YZy),(Xah,Cf,YZz),(Xah,Cf,YZaa),(Xah,Cf,YZab),(Xah,Cf,YZac),(Xah, Cf,YZad),(Xah,Cf,YZae),(Xah,Cf,YZaf),(Xah,Cf,YZag),(Xah,Cf,YZah),(Xah,Cf,YZai), (Xah,Cf,YZaj),(Xah,Cf,YZak),(Xah,Cf,YZal),(Xah,Cf,YZam),(Xah,Cf,YZan),(Xah, Cf,YZao),(Xah,Cf,YZap),(Xah,Cf,YZaq),(Xah,Cf,YZar),(Xah,Cf,YZas),(Xah,Cf,YZat), (Xah,Cf,YZau),(Xah,Cf,YZav),(Xah,Cf,YZaw),(Xah,Cf,YZax),(Xah,Cf,YZay),(Xah, Cf,YZaz),(Xah,Cf,YZba),(Xah,Cf,YZbb),(Xah,Cf,YZbc),(Xah,Cf,YZbd),(Xah,Cf,YZbe), (Xah,Cf,YZbf),(Xah,Cf,YZbg),(Xah,Cf,YZbh),(Xah,Cf,YZbi),(Xah,Cf,YZbj),(Xah, Cf,YZbk),(Xah,Cf,YZbl),(Xah,Cf,YZbm),(Xah,Cf,YZbn),(Xah,Cf,YZbo),(Xah,Cf,YZbp), (Xah,Cf,YZbq),(Xah,Cf,YZbr),(Xah,Cf,YZbs),(Xah,Cf,YZbt),(Xah,Cf,YZbu),(Xah, Cf,YZbv),(Xah,Cf,YZbw),(Xah,Cf,YZbx),(Xah,Cf,YZby),(Xah,Cf,YZbz),(Xah,Cf,YZca), (Xah,Cf,YZcb),(Xah,Cf,YZcc),(Xah,Cf,YZcd),(Xah,Cf,YZce),(Xah,Cf,YZcf),(Xah, Cf,YZcg),(Xah,Cf,YZch),(Xah,Cf,YZci),(Xah,Cf,YZcj),(Xah,Cf,YZck),(Xah,Cf,YZcl), (Xah,Cf,YZcm),(Xah,Cf,YZcn),(Xah,Cf,YZco),(Xah,Cf,YZcp),(Xah,Cf,YZcq),(Xah, Cf,YZcr),(Xah,Cf,YZcs),(Xah,Cf,YZct),(Xah,Cf,YZcu),(Xah,Cf,YZcv),(Xah,Cf, YZcw),(Xah,Cf,YZcx),(Xah,Cf,YZcy),(Xah,Cf,YZcz),(Xah,Cf,YZda),(Xah,Cf,YZdb), (Xah,Cf,YZdc),(Xah,Cf,YZdd),(Xah,Cf,YZde),(Xah,Cf,YZdf),(Xah,Cf,YZdg),(Xah,Cf, YZdh),(Xah,Cf,YZdi),(Xah,Cf,YZdj),(Xah,Cf,YZdk),(Xah,Cf,YZdl),(Xah,Cf,YZdm), (Xah,Cf,YZdn),(Xah,Cf,YZdo),(Xah,Cf,YZdp),(Xah,Cf,YZdq),(Xah,Cf,YZdr),(Xah, Cf,YZds),(Xah,Cf,Yzdt),(Xah,Cf,YZdu),(Xah,Cf,YZdv),(Xah,Cf,YZdw),(Xah,Cf,YZdx), (Xah,Cf,YZdy),(Xah,Cf,YZdz),(Xah,Cf,YZea),(Xah,Cf,YZeb),(Xah,Cf,YZec),(Xah, Cf,YZed),(Xah,Cf,YZee),(Xah,Cf,YZef),(Xah,Cf,YZeg),(Xah,Cf,YZeh),(Xah,Cf,YZei), (Xah,Cf,YZej),(Xah,Cf,YZek),(Xah,Cf,YZel),(Xah,Cf,YZem),(Xah,Cf,YZen),(Xah, Cf,YZeo),(Xah,Cf,YZep),(Xah,Cf,YZeq),(Xah,Cf,YZer),(Xah,Cf,YZes),(Xah,Cf,YZet), (Xah,Cf,YZeu),(Xah,Cf,YZev),(Xah,Cf,YZew),(Xah,Cf,YZex),(Xah,Cf,YZey),(Xah, Cf,YZez),(Xah,Cf,YZfa),(Xah,Cf,YZfb),(Xah,Cf,YZfc),(Xah,Cf,YZfd),(Xah,Cf,YZfe), (Xah,Cf,YZff),(Xah,Cf,YZfg),(Xah,Cf,YZfh),(Xah,Cf,YZfi),(Xah,Cf,YZfj),(Xah,Cf, YZfk),(Xah,Cf,YZfl),(Xah,Cf,YZfm),(Xah,Cf,YZfn),(Xah,Cf,YZfo),(Xah,Cf,YZfp),(Xah, Cf,YZfq),(Xah,Cf,YZfr),(Xah,Cf,YZfs),(Xah,Cf,YZft),(Xah,Cf,YZfu),(Xah,Cf,YZfv), (Xah,Cf,YZfw),(Xah,Cf,YZfx),(Xah,Cf,YZfy),(Xah,Cf,YZfz),(Xah,Cf,YZga),(Xah, Cf,YZgb),(Xah,Cf,YZgc),(Xah,Cf,YZgd),(Xah,Cf,YZge),(Xah,Cf,YZgf),(Xah,Cf,YZgg), (Xah,Cf,YZgh),(Xah,Cf,YZgi),(Xah,Cf,YZgj),(Xah,Cf,YZgk),(Xah,Cf,YZgl),(Xah, Cf,YZgm),(Xah,Cf,YZgn),(Xah,Cf,YZgo),(Xah,Cf,YZgp),(Xah,Cf,YZgq),(Xah,Cf, YZgr),(Xah,Cf,YZgs),(Xah,Cf,YZgt), (Xai,Ca,YZa),(Xai,Ca,YZb),(Xai,Ca,YZc),(Xai,Ca,YZd),(Xai,Ca,YZe),(Xai,Ca,YZf),(Xai, Ca,YZg),(Xai,Ca,YZh),(Xai,Ca,YZi),(Xai,Ca,YZj),(Xai,Ca,YZk),(Xai,Ca,YZl),(Xai, Ca,YZm),(Xai,Ca,YZn),(Xai,Ca,YZo),(Xai,Ca,YZp),(Xai,Ca,YZq),(Xai,Ca,YZr),(Xai, Ca,YZs),(Xai,Ca,YZt),(Xai,Ca,YZu),(Xai,Ca,YZv),(Xai,Ca,YZw),(Xai,Ca,YZx),(Xai, Ca,YZy),(Xai,Ca,YZz),(Xai,Ca,YZaa),(Xai,Ca,YZab),(Xai,Ca,YZac),(Xai,Ca,YZad), (Xai,Ca,YZae),(Xai,Ca,YZaf),(Xai,Ca,YZag),(Xai,Ca,YZah),(Xai,Ca,YZai),(Xai,Ca, YZaj),(Xai,Ca,YZak),(Xai,Ca,YZal),(Xai,Ca,YZam),(Xai,Ca,YZan),(Xai,Ca,YZao),(Xai, Ca,YZap),(Xai,Ca,YZaq),(Xai,Ca,YZar),(Xai,Ca,YZas),(Xai,Ca,YZat),(Xai,Ca,YZau), (Xai,Ca,YZav),(Xai,Ca,YZaw),(Xai,Ca,YZax),(Xai,Ca,YZay),(Xai,Ca,YZaz),(Xai, Ca,YZba),(Xai,Ca,YZbb),(Xai,Ca,YZbc),(Xai,Ca,YZbd),(Xai,Ca,YZbe),(Xai,Ca,YZbf), (Xai,Ca,YZbg),(Xai,Ca,YZbh),(Xai,Ca,YZbi),(Xai,Ca,YZbj),(Xai,Ca,YZbk),(Xai,Ca, YZbl),(Xai,Ca,YZbm),(Xai,Ca,YZbn),(Xai,Ca,YZbo),(Xai,Ca,YZbp),(Xai,Ca,YZbq), (Xai,Ca,YZbr),(Xai,Ca,YZbs),(Xai,Ca,YZbt),(Xai,Ca,YZbu),(Xai,Ca,YZbv),(Xai,Ca, YZbw),(Xai,Ca,YZbx),(Xai,Ca,YZby),(Xai,Ca,YZbz),(Xai,Ca,YZca),(Xai,Ca,YZcb),(Xai, Ca,YZcc),(Xai,Ca,YZcd),(Xai,Ca,YZce),(Xai,Ca,YZcf),(Xai,Ca,YZcg),(Xai,Ca,YZch), (Xai,Ca,YZci),(Xai,Ca,YZcj),(Xai,Ca,YZck),(Xai,Ca,YZcl),(Xai,Ca,YZcm),(Xai, Ca,YZcn),(Xai,Ca,YZco),(Xai,Ca,YZcp),(Xai,Ca,YZcq),(Xai,Ca,YZcr),(Xai,Ca,YZcs), (Xai,Ca,YZct),(Xai,Ca,YZcu),(Xai,Ca,YZcv),(Xai,Ca,YZcw),(Xai,Ca,YZcx),(Xai,Ca, YZcy),(Xai,Ca,YZcz),(Xai,Ca,YZda),(Xai,Ca,YZdb),(Xai,Ca,YZdc),(Xai,Ca,YZdd),(Xai, Ca,YZde),(Xai,Ca,YZdf),(Xai,Ca,YZdg),(Xai,Ca,YZdh),(Xai,Ca,YZdi),(Xai,Ca,YZdj), (Xai,Ca,YZdk),(Xai,Ca,YZdl),(Xai,Ca,YZdm),(Xai,Ca,YZdn),(Xai,Ca,YZdo),(Xai, Ca,YZdp),(Xai,Ca,YZdq),(Xai,Ca,YZdr),(Xai,Ca,YZds),(Xai,Ca,Yzdt),(Xai,Ca,YZdu), (Xai,Ca,YZdv),(Xai,Ca,YZdw),(Xai,Ca,YZdx),(Xai,Ca,YZdy),(Xai,Ca,YZdz),(Xai, Ca,YZea),(Xai,Ca,YZeb),(Xai,Ca,YZec),(Xai,Ca,YZed),(Xai,Ca,YZee),(Xai,Ca,YZef), (Xai,Ca,YZeg),(Xai,Ca,YZeh),(Xai,Ca,YZei),(Xai,Ca,YZej),(Xai,Ca,YZek),(Xai,Ca, YZel),(Xai,Ca,YZem),(Xai,Ca,YZen),(Xai,Ca,YZeo),(Xai,Ca,YZep),(Xai,Ca,YZeq),(Xai, Ca,YZer),(Xai,Ca,YZes),(Xai,Ca,YZet),(Xai,Ca,YZeu),(Xai,Ca,YZev),(Xai,Ca,YZew), (Xai,Ca,YZex),(Xai,Ca,YZey),(Xai,Ca,YZez),(Xai,Ca,YZfa),(Xai,Ca,YZfb),(Xai, Ca,YZfc),(Xai,Ca,YZfd),(Xai,Ca,YZfe),(Xai,Ca,YZff),(Xai,Ca,YZfg),(Xai,Ca,YZfh), (Xai,Ca,YZfi),(Xai,Ca,YZfj),(Xai,Ca,YZfk),(Xai,Ca,YZfl),(Xai,Ca,YZfm),(Xai,Ca,YZfn), (Xai,Ca,YZfo),(Xai,Ca,YZfp),(Xai,Ca,YZfq),(Xai,Ca,YZfr),(Xai,Ca,YZfs),(Xai, Ca,YZft),(Xai,Ca,YZfu),(Xai,Ca,YZfv),(Xai,Ca,YZfw),(Xai,Ca,YZfx),(Xai,Ca,YZfy), (Xai,Ca,YZfz),(Xai,Ca,YZga),(Xai,Ca,YZgb),(Xai,Ca,YZgc),(Xai,Ca,YZgd),(Xai,Ca, YZge),(Xai,Ca,YZgf),(Xai,Ca,YZgg),(Xai,Ca,YZgh),(Xai,Ca,YZgi),(Xai,Ca,YZgj),(Xai, Ca,YZgk),(Xai,Ca,YZgl),(Xai,Ca,YZgm),(Xai,Ca,YZgn),(Xai,Ca,YZgo),(Xai,Ca,YZgp), (Xai,Ca,YZgq),(Xai,Ca,YZgr),(Xai,Ca,YZgs),(Xai,Ca,YZgt),(Xai,Cb,YZa),(Xai, Cb,YZb),(Xai,Cb,YZc),(Xai,Cb,YZd),(Xai,Cb,YZe),(Xai,Cb,YZf),(Xai,Cb,YZg),(Xai, Cb,YZh),(Xai,Cb,YZi),(Xai,Cb,YZj),(Xai,Cb,YZk),(Xai,Cb,YZl),(Xai,Cb,YZm),(Xai, Cb,YZn),(Xai,Cb,YZo),(Xai,Cb,YZp),(Xai,Cb,YZq),(Xai,Cb,YZr),(Xai,Cb,YZs),(Xai, Cb,YZt),(Xai,Cb,YZu),(Xai,Cb,YZv),(Xai,Cb,YZw),(Xai,Cb,YZx),(Xai,Cb,YZy),(Xai, Cb,YZz),(Xai,Cb,YZaa),(Xai,Cb,YZab),(Xai,Cb,YZac),(Xai,Cb,YZad),(Xai,Cb,YZae), (Xai,Cb,YZaf),(Xai,Cb,YZag),(Xai,Cb,YZah),(Xai,Cb,YZai),(Xai,Cb,YZaj),(Xai,Cb, YZak),(Xai,Cb,YZal),(Xai,Cb,YZam),(Xai,Cb,YZan),(Xai,Cb,YZao),(Xai,Cb,YZap),(Xai, Cb,YZaq),(Xai,Cb,YZar),(Xai,Cb,YZas),(Xai,Cb,YZat),(Xai,Cb,YZau),(Xai,Cb,YZav), (Xai,Cb,YZaw),(Xai,Cb,YZax),(Xai,Cb,YZay),(Xai,Cb,YZaz),(Xai,Cb,YZba),(Xai, Cb,YZbb),(Xai,Cb,YZbc),(Xai,Cb,YZbd),(Xai,Cb,YZbe),(Xai,Cb,YZbf),(Xai,Cb,YZbg), (Xai,Cb,YZbh),(Xai,Cb,YZbi),(Xai,Cb,YZbj),(Xai,Cb,YZbk),(Xai,Cb,YZbl),(Xai, Cb,YZbm),(Xai,Cb,YZbn),(Xai,Cb,YZbo),(Xai,Cb,YZbp),(Xai,Cb,YZbq),(Xai,Cb,YZbr), (Xai,Cb,YZbs),(Xai,Cb,YZbt),(Xai,Cb,YZbu),(Xai,Cb,YZbv),(Xai,Cb,YZbw),(Xai, Cb,YZbx),(Xai,Cb,YZby),(Xai,Cb,YZbz),(Xai,Cb,YZca),(Xai,Cb,YZcb),(Xai,Cb,YZcc), (Xai,Cb,YZcd),(Xai,Cb,YZce),(Xai,Cb,YZcf),(Xai,Cb,YZcg),(Xai,Cb,YZch),(Xai, Cb,YZci),(Xai,Cb,YZcj),(Xai,Cb,YZck),(Xai,Cb,YZcl),(Xai,Cb,YZcm),(Xai,Cb,YZcn), (Xai,Cb,YZco),(Xai,Cb,YZcp),(Xai,Cb,YZcq),(Xai,Cb,YZcr),(Xai,Cb,YZcs),(Xai, Cb,YZct),(Xai,Cb,YZcu),(Xai,Cb,YZcv),(Xai,Cb,YZcw),(Xai,Cb,YZcx),(Xai,Cb,YZcy), (Xai,Cb,YZcz),(Xai,Cb,YZda),(Xai,Cb,YZdb),(Xai,Cb,YZdc),(Xai,Cb,YZdd),(Xai, Cb,YZde),(Xai,Cb,YZdf),(Xai,Cb,YZdg),(Xai,Cb,YZdh),(Xai,Cb,YZdi),(Xai,Cb,YZdj), (Xai,Cb,YZdk),(Xai,Cb,YZdl),(Xai,Cb,YZdm),(Xai,Cb,YZdn),(Xai,Cb,YZdo),(Xai, Cb,YZdp),(Xai,Cb,YZdq),(Xai,Cb,YZdr),(Xai,Cb,YZds),(Xai,Cb,Yzdt),(Xai,Cb,YZdu), (Xai,Cb,YZdv),(Xai,Cb,YZdw),(Xai,Cb,YZdx),(Xai,Cb,YZdy),(Xai,Cb,YZdz),(Xai, Cb,YZea),(Xai,Cb,YZeb),(Xai,Cb,YZec),(Xai,Cb,YZed),(Xai,Cb,YZee),(Xai,Cb,YZef), (Xai,Cb,YZeg),(Xai,Cb,YZeh),(Xai,Cb,YZei),(Xai,Cb,YZej),(Xai,Cb,YZek),(Xai,Cb, YZel),(Xai,Cb,YZem),(Xai,Cb,YZen),(Xai,Cb,YZeo),(Xai,Cb,YZep),(Xai,Cb,YZeq), (Xai,Cb,YZer),(Xai,Cb,YZes),(Xai,Cb,YZet),(Xai,Cb,YZeu),(Xai,Cb,YZev),(Xai,Cb, YZew),(Xai,Cb,YZex),(Xai,Cb,YZey),(Xai,Cb,YZez),(Xai,Cb,YZfa),(Xai,Cb,YZfb),(Xai, Cb,YZfc),(Xai,Cb,YZfd),(Xai,Cb,YZfe),(Xai,Cb,YZff),(Xai,Cb,YZfg),(Xai,Cb,YZfh), (Xai,Cb,YZfi),(Xai,Cb,YZfj),(Xai,Cb,YZfk),(Xai,Cb,YZfl),(Xai,Cb,YZfm),(Xai, Cb,YZfn),(Xai,Cb,YZfo),(Xai,Cb,YZfp),(Xai,Cb,YZfq),(Xai,Cb,YZfr),(Xai,Cb,YZfs), (Xai,Cb,YZft),(Xai,Cb,YZfu),(Xai,Cb,YZfv),(Xai,Cb,YZfw),(Xai,Cb,YZfx),(Xai,Cb, YZfy),(Xai,Cb,YZfz),(Xai,Cb,YZga),(Xai,Cb,YZgb),(Xai,Cb,YZgc),(Xai,Cb,YZgd),(Xai, Cb,YZge),(Xai,Cb,YZgf),(Xai,Cb,YZgg),(Xai,Cb,YZgh),(Xai,Cb,YZgi),(Xai,Cb, YZgj),(Xai,Cb,YZgk),(Xai,Cb,YZgl),(Xai,Cb,YZgm),(Xai,Cb,YZgn),(Xai,Cb,YZgo),(Xai, Cb,YZgp),(Xai,Cb,YZgq),(Xai,Cb,YZgr),(Xai,Cb,YZgs),(Xai,Cb,YZgt),(Xai,Cc, YZa),(Xai,Cc,YZb),(Xai,Cc,YZc),(Xai,Cc,YZd),(Xai,Cc,YZe),(Xai,Cc,YZf),(Xai,Cc, YZg),(Xai,Cc,YZh),(Xai,Cc,YZi),(Xai,Cc,YZj),(Xai,Cc,YZk),(Xai,Cc,YZl),(Xai,Cc,YZm), (Xai,Cc,YZn),(Xai,Cc,YZo),(Xai,Cc,YZp),(Xai,Cc,YZq),(Xai,Cc,YZr),(Xai,Cc,YZs), (Xai,Cc,YZt),(Xai,Cc,YZu),(Xai,Cc,YZv),(Xai,Cc,YZw),(Xai,Cc,YZx),(Xai,Cc,YZy), (Xai,Cc,YZz),(Xai,Cc,YZaa),(Xai,Cc,YZab),(Xai,Cc,YZac),(Xai,Cc,YZad),(Xai, Cc,YZae),(Xai,Cc,YZaf),(Xai,Cc,YZag),(Xai,Cc,YZah),(Xai,Cc,YZai),(Xai,Cc,YZaj), (Xai,Cc,YZak),(Xai,Cc,YZal),(Xai,Cc,YZam),(Xai,Cc,YZan),(Xai,Cc,YZao),(Xai,Cc, YZap),(Xai,Cc,YZaq),(Xai,Cc,YZar),(Xai,Cc,YZas),(Xai,Cc,YZat),(Xai,Cc,YZau),(Xai, Cc,YZav),(Xai,Cc,YZaw),(Xai,Cc,YZax),(Xai,Cc,YZay),(Xai,Cc,YZaz),(Xai,Cc,YZba), (Xai,Cc,YZbb),(Xai,Cc,YZbc),(Xai,Cc,YZbd),(Xai,Cc,YZbe),(Xai,Cc,YZbf),(Xai, Cc,YZbg),(Xai,Cc,YZbh),(Xai,Cc,YZbi),(Xai,Cc,YZbj),(Xai,Cc,YZbk),(Xai,Cc,YZbl), (Xai,Cc,YZbm),(Xai,Cc,YZbn),(Xai,Cc,YZbo),(Xai,Cc,YZbp),(Xai,Cc,YZbq),(Xai, Cc,YZbr),(Xai,Cc,YZbs),(Xai,Cc,YZbt),(Xai,Cc,YZbu),(Xai,Cd,YZbv),(Xai,Cc,YZbw), (Xai,Cc,YZbx),(Xai,Cc,YZby),(Xai,Cc,YZbz),(Xai,Cc,YZca),(Xai,Cc,YZcb),(Xai,Cc, YZcc),(Xai,Cc,YZcd),(Xai,Cc,YZce),(Xai,Cc,YZcf),(Xai,Cc,YZcg),(Xai,Cc,YZch),(Xai, Cc,YZci),(Xai,Cc,YZcj),(Xai,Cc,YZck),(Xai,Cc,YZcl),(Xai,Cc,YZcm),(Xai,Cc,YZcn), (Xai,Cc,YZco),(Xai,Cc,YZcp),(Xai,Cc,YZcq),(Xai,Cc,YZcr),(Xai,Cc,YZcs),(Xai, Cc,YZct),(Xai,Cc,YZcu),(Xai,Cc,YZcv),(Xai,Cc,YZcw),(Xai,Cc,YZcx),(Xai,Cc,YZcy), (Xai,Cc,YZcz),(Xai,Cc,YZda),(Xai,Cc,YZdb),(Xai,Cc,YZdc),(Xai,Cc,YZdd),(Xai, Cc,YZde),(Xai,Cc,YZdf),(Xai,Cc,YZdg),(Xai,Cc,YZdh),(Xai,Cc,YZdi),(Xai,Cc,YZdj), (Xai,Cc,YZdk),(Xai,Cc,YZdl),(Xai,Cc,YZdm),(Xai,Cc,YZdn),(Xai,Cc,YZdo),(Xai,Cc, YZdp),(Xai,Cc,YZdq),(Xai,Cc,YZdr),(Xai,Cc,YZds),(Xai,Cc,Yzdt),(Xai,Cc,YZdu),(Xai, Cc,YZdv),(Xai,Cc,YZdw),(Xai,Cc,YZdx),(Xai,Cc,YZdy),(Xai,Cc,YZdz),(Xai,Cc, YZea),(Xai,Cc,YZeb),(Xai,Cc,YZec),(Xai,Cc,YZed),(Xai,Cc,YZee),(Xai,Cc,YZef),(Xai, Cc,YZeg),(Xai,Cc,YZeh),(Xai,Cc,YZei),(Xai,Cc,YZej),(Xai,Cc,YZek),(Xai,Cc,YZel), (Xai,Cc,YZem),(Xai,Cc,YZen),(Xai,Cc,YZeo),(Xai,Cc,YZep),(Xai,Cc,YZeq),(Xai, Cc,YZer),(Xai,Cc,YZes),(Xai,Cc,YZet),(Xai,Cc,YZeu),(Xai,Cc,YZev),(Xai,Cc,YZew), (Xai,Cc,YZex),(Xai,Cc,YZey),(Xai,Cc,YZez),(Xai,Cc,YZfa),(Xai,Cc,YZfb),(Xai,Cc, YZfc),(Xai,Cc,YZfd),(Xai,Cc,YZfe),(Xai,Cc,YZff),(Xai,Cc,YZfg),(Xai,Cc,YZfh),(Xai, Cc,YZfi),(Xai,Cc,YZfj),(Xai,Cc,YZfk),(Xai,Cc,YZfl),(Xai,Cc,YZfm),(Xai,Cc,YZfn),(Xai, Cc,YZfo),(Xai,Cc,YZfp),(Xai,Cc,YZfq),(Xai,Cc,YZfr),(Xai,Cc,YZfs),(Xai,Cc,YZft), (Xai,Cc,YZfu),(Xai,Cc,YZfv),(Xai,Cc,YZfw),(Xai,Cc,YZfx),(Xai,Cc,YZfy),(Xai,Cc, YZfz),(Xai,Cc,YZga),(Xai,Cc,YZgb),(Xai,Cc,YZgc),(Xai,Cc,YZgd),(Xai,Cc,YZge), (Xai,Cc,YZgf),(Xai,Cc,YZgg),(Xai,Cc,YZgh),(Xai,Cc,YZgi),(Xai,Cc,YZgj),(Xai,Cc, YZgk),(Xai,Cc,YZgl),(Xai,Cc,YZgm),(Xai,Cc,YZgn),(Xai,Cc,YZgo),(Xai,Cc,YZgp),(Xai, Cc,YZgq),(Xai,Cc,YZgr),(Xai,Cc,YZgs),(Xai,Cc,YZgt),(Xai,Cd,YZa),(Xai,Cd,YZb), (Xai,Cd,YZc),(Xai,Cd,YZd),(Xai,Cd,YZe),(Xai,Cd,YZf),(Xai,Cd,YZg),(Xai,Cd,YZh), (Xai,Cd,YZi),(Xai,Cd,YZj),(Xai,Cd,YZk),(Xai,Cd,YZl),(Xai,Cd,YZm),(Xai,Cd,YZn), (Xai,Cd,YZo),(Xai,Cd,YZp),(Xai,Cd,YZq),(Xai,Cd,YZr),(Xai,Cd,YZs),(Xai,Cd,YZt), (Xai,Cd,YZu),(Xai,Cd,YZv),(Xai,Cd,YZw),(Xai,Cd,YZx),(Xai,Cd,YZy),(Xai,Cd,YZz), (Xai,Cd,YZaa),(Xai,Cd,YZab),(Xai,Cd,YZac),(Xai,Cd,YZad),(Xai,Cd,YZae),(Xai, Cd,YZaf),(Xai,Cd,YZag),(Xai,Cd,YZah),(Xai,Cd,YZai),(Xai,Cd,YZaj),(Xai,Cd,YZak), (Xai,Cd,YZal),(Xai,Cd,YZam),(Xai,Cd,YZan),(Xai,Cd,YZao),(Xai,Cd,YZap),(Xai,Cd, YZaq),(Xai,Cd,YZar),(Xai,Cd,YZas),(Xai,Cd,YZat),(Xai,Cd,YZau),(Xai,Cd,YZav),(Xai, Cd,YZaw),(Xai,Cd,YZax),(Xai,Cd,YZay),(Xai,Cd,YZaz),(Xai,Cd,YZba),(Xai,Cd, YZbb),(Xai,Cd,YZbc),(Xai,Cd,YZbd),(Xai,Cd,YZbe),(Xai,Cd,YZbf),(Xai,Cd,YZbg),(Xai, Cd,YZbh),(Xai,Cd,YZbi),(Xai,Cd,YZbj),(Xai,Cd,YZbk),(Xai,Cd,YZbl),(Xai,Cd,YZbm), (Xai,Cd,YZbn),(Xai,Cd,YZbo),(Xai,Cd,YZbp),(Xai,Cd,YZbq),(Xai,Cd,YZbr),(Xai, Cd,YZbs),(Xai,Cd,YZbt),(Xai,Cd,YZbu),(Xai,Cd,YZbv),(Xai,Cd,YZbw),(Xai,Cd, YZbx),(Xai,Cd,YZby),(Xai,Cd,YZbz),(Xai,Cd,YZca),(Xai,Cd,YZcb),(Xai,Cd,YZcc),(Xai, Cd,YZcd),(Xai,Cd,YZce),(Xai,Cd,YZcf),(Xai,Cd,YZcg),(Xai,Cd,YZch),(Xai,Cd, YZci),(Xai,Cd,YZcj),(Xai,Cd,YZck),(Xai,Cd,YZcl),(Xai,Cd,YZcm),(Xai,Cd,YZcn),(Xai, Cd,YZco),(Xai,Cd,YZcp),(Xai,Cd,YZcq),(Xai,Cd,YZer),(Xai,Cd,YZcs),(Xai,Cd, YZct),(Xai,Cd,YZcu),(Xai,Cd,YZcv),(Xai,Cd,YZcw),(Xai,Cd,YZcx),(Xai,Cd,YZcy),(Xai, Cd,YZcz),(Xai,Cd,YZda),(Xai,Cd,YZdb),(Xai,Cd,YZdc),(Xai,Cd,YZdd),(Xai,Cd, YZde),(Xai,Cd,YZdf),(Xai,Cd,YZdg),(Xai,Cd,YZdh),(Xai,Cd,YZdi),(Xai,Cd,YZdj),(Xai, Cd,YZdk),(Xai,Cd,YZdl),(Xai,Cd,YZdm),(Xai,Cd,YZdn),(Xai,Cd,YZdo),(Xai,Cd, YZdp),(Xai,Cd,YZdq),(Xai,Cd,YZdr),(Xai,Cd,YZds),(Xai,Cd,Yzdt),(Xai,Cd,YZdu),(Xai, Cd,YZdv),(Xai,Cd,YZdw),(Xai,Cd,YZdx),(Xai,Cd,YZdy),(Xai,Cd,YZdz),(Xai,Cd, YZea),(Xai,Cd,YZeb),(Xai,Cd,YZec),(Xai,Cd,YZed),(Xai,Cd,YZee),(Xai,Cd,YZef),(Xai, Cd,YZeg),(Xai,Cd,YZeh),(Xai,Cd,YZei),(Xai,Cd,YZej),(Xai,Cd,YZek),(Xai,Cd,YZel), (Xai,Cd,YZem),(Xai,Cd,YZen),(Xai,Cd,YZeo),(Xai,Cd,YZep),(Xai,Cd,YZeq),(Xai, Cd,YZer),(Xai,Cd,YZes),(Xai,Cd,YZet),(Xai,Cd,YZeu),(Xai,Cd,YZev),(Xai,Cd,YZew), (Xai,Cd,YZex),(Xai,Cd,YZey),(Xai,Cd,YZez),(Xai,Cd,YZfa),(Xai,Cd,YZfb),(Xai, Cd,YZfc),(Xai,Cd,YZfd),(Xai,Cd,YZfe),(Xai,Cd,YZff),(Xai,Cd,YZfg),(Xai,Cd,YZfh), (Xai,Cd,YZfi),(Xai,Cd,YZfj),(Xai,Cd,YZfk),(Xai,Cd,YZfl),(Xai,Cd,YZfm),(Xai,Cd,YZfn), (Xai,Cd,YZfo),(Xai,Cd,YZfp),(Xai,Cd,YZfq),(Xai,Cd,YZfr),(Xai,Cd,YZfs),(Xai, Cd,YZft),(Xai,Cd,YZfu),(Xai,Cd,YZfv),(Xai,Cd,YZfw),(Xai,Cd,YZfx),(Xai,Cd,YZfy), (Xai,Cd,YZfz),(Xai,Cd,YZga),(Xai,Cd,YZgb),(Xai,Cd,YZgc),(Xai,Cd,YZgd),(Xai,Cd, YZge),(Xai,Cd,YZgf),(Xai,Cd,YZgg),(Xai,Cd,YZgh),(Xai,Cd,YZgi),(Xai,Cd,YZgj),(Xai, Cd,YZgk),(Xai,Cd,YZgl),(Xai,Cd,YZgm),(Xai,Cd,YZgn),(Xai,Cd,YZgo),(Xai,Cd, YZgp),(Xai,Cd,YZgq),(Xai,Cd,YZgr),(Xai,Cd,YZgs),(Xai,Cd,YZgt),(Xai,Ce,YZa),(Xai, Ce,YZb),(Xai,Ce,YZc),(Xai,Ce,YZd),(Xai,Ce,YZe),(Xai,Ce,YZf),(Xai,Ce,YZg),(Xai, Ce,YZh),(Xai,Ce,YZi),(Xai,Ce,YZj),(Xai,Ce,YZk),(Xai,Ce,YZl),(Xai,Ce,YZm),(Xai, Ce,YZn),(Xai,Ce,YZo),(Xai,Ce,YZp),(Xai,Ce,YZq),(Xai,Ce,YZr),(Xai,Ce,YZs),(Xai, Ce,YZt),(Xai,Ce,YZu),(Xai,Ce,YZv),(Xai,Ce,YZw),(Xai,Ce,YZx),(Xai,Ce,YZy),(Xai, Ce,YZz),(Xai,Ce,YZaa),(Xai,Ce,YZab),(Xai,Ce,YZac),(Xai,Ce,YZad),(Xai,Ce,YZae), (Xai,Ce,YZaf),(Xai,Ce,YZag),(Xai,Ce,YZah),(Xai,Ce,YZai),(Xai,Ce,YZaj),(Xai,Ce,YZak), (Xai,Ce,YZal),(Xai,Ce,YZam),(Xai,Ce,YZan),(Xai,Ce,YZao),(Xai,Ce,YZap),(Xai, Ce,YZaq),(Xai,Ce,YZar),(Xai,Ce,YZas),(Xai,Ce,YZat),(Xai,Ce,YZau),(Xai,Ce,YZav), (Xai,Ce,YZaw),(Xai,Ce,YZax),(Xai,Ce,YZay),(Xai,Ce,YZaz),(Xai,Ce,YZba),(Xai,Ce, YZbb),(Xai,Ce,YZbc),(Xai,Ce,YZbd),(Xai,Ce,YZbe),(Xai,Ce,YZbf),(Xai,Ce,YZbg), (Xai,Ce,YZbh),(Xai,Ce,YZbi),(Xai,Ce,YZbj),(Xai,Ce,YZbk),(Xai,Ce,YZbl),(Xai,Ce,YZbm), (Xai,Ce,YZbn),(Xai,Ce,YZbo),(Xai,Ce,YZbp),(Xai,Ce,YZbq),(Xai,Ce,YZbr),(Xai, Ce,YZbs),(Xai,Ce,YZbt),(Xai,Ce,YZbu),(Xai,Ce,YZbv),(Xai,Ce,YZbw),(Xai,Ce,YZbx), (Xai,Ce,YZby),(Xai,Ce,YZbz),(Xai,Ce,YZca),(Xai,Ce,YZcb),(Xai,Ce,YZcc),(Xai, Ce,YZcd),(Xai,Ce,YZce),(Xai,Ce,YZcf),(Xai,Ce,YZcg),(Xai,Ce,YZch),(Xai,Ce,YZci), (Xai,Ce,YZcj),(Xai,Ce,YZck),(Xai,Ce,YZcl),(Xai,Ce,YZcm),(Xai,Ce,YZcn),(Xai,Ce, YZco),(Xai,Ce,YZcp),(Xai,Ce,YZcq),(Xai,Ce,YZcr),(Xai,Ce,YZcs),(Xai,Ce,YZct),(Xai, Ce,YZcu),(Xai,Ce,YZcv),(Xai,Ce,YZcw),(Xai,Ce,YZcx),(Xai,Ce,YZcy),(Xai,Ce,YZcz), (Xai,Ce,YZda),(Xai,Ce,YZdb),(Xai,Ce,YZdc),(Xai,Ce,YZdd),(Xai,Ce,YZde),(Xai, Ce,YZdf),(Xai,Ce,YZdg),(Xai,Ce,YZdh),(Xai,Ce,YZdi),(Xai,Ce,YZdj),(Xai,Ce,YZdk), (Xai,Ce,YZdl),(Xai,Ce,YZdm),(Xai,Ce,YZdn),(Xai,Ce,YZdo),(Xai,Ce,YZdp),(Xai, Ce,YZdq),(Xai,Ce,YZdr),(Xai,Ce,YZds),(Xai,Ce,Yzdt),(Xai,Ce,YZdu),(Xai,Ce,YZdv), (Xai,Ce,YZdw),(Xai,Ce,YZdx),(Xai,Ce,YZdy),(Xai,Ce,YZdz),(Xai,Ce,YZea),(Xai,Ce, YZeb),(Xai,Ce,YZec),(Xai,Ce,YZed),(Xai,Ce,YZee),(Xai,Ce,YZef),(Xai,Ce,YZeg),(Xai, Ce,YZeh),(Xai,Ce,YZei),(Xai,Ce,YZej),(Xai,Ce,YZek),(Xai,Ce,YZel),(Xai,Ce,YZem), (Xai,Ce,YZen),(Xai,Ce,YZeo),(Xai,Ce,YZep),(Xai,Ce,YZeq),(Xai,Ce,YZer),(Xai, Ce,YZes),(Xai,Ce,YZet),(Xai,Ce,YZeu),(Xai,Ce,YZev),(Xai,Ce,YZew),(Xai,Ce,YZex), (Xai,Ce,YZey),(Xai,Ce,YZez),(Xai,Ce,YZfa),(Xai,Ce,YZfb),(Xai,Ce,YZfc),(Xai,Ce, YZfd),(Xai,Ce,YZfe),(Xai,Ce,YZff),(Xai,Ce,YZfg),(Xai,Ce,YZfh),(Xai,Ce,YZfi),(Xai, Ce,YZfj),(Xai,Ce,YZfk),(Xai,Ce,YZfl),(Xai,Ce,YZfm),(Xai,Ce,YZfn),(Xai,Ce,YZfo), (Xai,Ce,YZfp),(Xai,Ce,YZfq),(Xai,Ce,YZfr),(Xai,Ce,YZfs),(Xai,Ce,YZft),(Xai,Ce,YZfu), (Xai,Ce,YZfv),(Xai,Ce,YZfw),(Xai,Ce,YZfx),(Xai,Ce,YZfy),(Xai,Ce,YZfz),(Xai,Ce, YZga),(Xai,Ce,YZgb),(Xai,Ce,YZgc),(Xai,Ce,YZgd),(Xai,Ce,YZge),(Xai,Ce,YZgf), (Xai,Ce,YZgg),(Xai,Ce,YZgh),(Xai,Ce,YZgi),(Xai,Ce,YZgj),(Xai,Ce,YZgk),(Xai,Ce, YZgl),(Xai,Ce,YZgm),(Xai,Ce,YZgn),(Xai,Ce,YZgo),(Xai,Ce,YZgp),(Xai,Ce,YZgq),(Xai, Ce,YZgr),(Xai,Ce,YZgs),(Xai,Ce,YZgt),(Xai,Cf,YZa),(Xai,Cf,YZb),(Xai,Cf,YZc), (Xai,Cf,YZd),(Xai,Cf,YZe),(Xai,Cf,YZf),(Xai,Cf,YZg),(Xai,Cf,YZh),(Xai,Cf,YZi),(Xai, Cf,YZj),(Xai,Cf,YZk),(Xai,Cf,YZl),(Xai,Cf,YZm),(Xai,Cf,YZn),(Xai,Cf,YZo),(Xai, Cf,YZp),(Xai,Cf,YZq),(Xai,Cf,YZr),(Xai,Cf,YZs),(Xai,Cf,YZt),(Xai,Cf,YZu),(Xai, Cf,YZv),(Xai,Cf,YZw),(Xai,Cf,YZx),(Xai,Cf,YZy),(Xai,Cf,YZz),(Xai,Cf,YZaa),(Xai, Cf,YZab),(Xai,Cf,YZac),(Xai,Cf,YZad),(Xai,Cf,YZae),(Xai,Cf,YZaf),(Xai,Cf,YZag),(Xai, Cf,YZah),(Xai,Cf,YZai),(Xai,Cf,YZaj),(Xai,Cf,YZak),(Xai,Cf,YZal),(Xai,Cf,YZam), (Xai,Cf,YZan),(Xai,Cf,YZao),(Xai,Cf,YZap),(Xai,Cf,YZaq),(Xai,Cf,YZar),(Xai,Cf, YZas),(Xai,Cf,YZat),(Xai,Cf,YZau),(Xai,Cf,YZav),(Xai,Cf,YZaw),(Xai,Cf,YZax),(Xai, Cf,YZay),(Xai,Cf,YZaz),(Xai,Cf,YZba),(Xai,Cf,YZbb),(Xai,Cf,YZbc),(Xai,Cf,YZbd), (Xai,Cf,YZbe),(Xai,Cf,YZbf),(Xai,Cf,YZbg),(Xai,Cf,YZbh),(Xai,Cf,YZbi),(Xai,Cf, YZbj),(Xai,Cf,YZbk),(Xai,Cf,YZbl),(Xai,Cf,YZbm),(Xai,Cf,YZbn),(Xai,Cf,YZbo),(Xai, Cf,YZbp),(Xai,Cf,YZbq),(Xai,Cf,YZbr),(Xai,Cf,YZbs),(Xai,Cf,YZbt),(Xai,Cf,YZbu), (Xai,Cf,YZbv),(Xai,Cf,YZbw),(Xai,Cf,YZbx),(Xai,Cf,YZby),(Xai,Cf,YZbz),(Xai,Cf, YZca),(Xai,Cf,YZcb),(Xai,Cf,YZcc),(Xai,Cf,YZcd),(Xai,Cf,YZce),(Xai,Cf,YZcf),(Xai, Cf,YZcg),(Xai,Cf,YZch),(Xai,Cf,YZci),(Xai,Cf,YZcj),(Xai,Cf,YZck),(Xai,Cf,YZcl), (Xai,Cf,YZcm),(Xai,Cf,YZcn),(Xai,Cf,YZco),(Xai,Cf,YZcp),(Xai,Cf,YZcq),(Xai,Cf, YZcr),(Xai,Cf,YZcs),(Xai,Cf,YZct),(Xai,Cf,YZcu),(Xai,Cf,YZcv),(Xai,Cf,YZcw),(Xai, Cf,YZcx),(Xai,Cf,YZcy),(Xai,Cf,YZcz),(Xai,Cf,YZda),(Xai,Cf,YZdb),(Xai,Cf,YZdc), (Xai,Cf,YZdd),(Xai,Cf,YZde),(Xai,Cf,YZdf),(Xai,Cf,YZdg),(Xai,Cf,YZdh),(Xai,Cf, YZdi),(Xai,Cf,YZdj),(Xai,Cf,YZdk),(Xai,Cf,YZdl),(Xai,Cf,YZdm),(Xai,Cf,YZdn),(Xai, Cf,YZdo),(Xai,Cf,YZdp),(Xai,Cf,YZdq),(Xai,Cf,YZdr),(Xai,Cf,YZds),(Xai,Cf,Yzdt), (Xai,Cf,YZdu),(Xai,Cf,YZdv),(Xai,Cf,YZdw),(Xai,Cf,YZdx),(Xai,Cf,YZdy),(Xai,Cf, YZdz),(Xai,Cf,YZea),(Xai,Cf,YZeb),(Xai,Cf,YZec),(Xai,Cf,YZed),(Xai,Cf,YZee),(Xai, Cf,YZef),(Xai,Cf,YZeg),(Xai,Cf,YZeh),(Xai,Cf,YZei),(Xai,Cf,YZej),(Xai,Cf,YZek), (Xai,Cf,YZel),(Xai,Cf,YZem),(Xai,Cf,YZen),(Xai,Cf,YZeo),(Xai,Cf,YZep),(Xai,Cf,YZeq), (Xai,Cf,YZer),(Xai,Cf,YZes),(Xai,Cf,YZet),(Xai,Cf,YZeu),(Xai,Cf,YZev),(Xai, Cf,YZew),(Xai,Cf,YZex),(Xai,Cf,YZey),(Xai,Cf,YZez),(Xai,Cf,YZfa),(Xai,Cf,YZfb), (Xai,Cf,YZfc),(Xai,Cf,YZfd),(Xai,Cf,YZfe),(Xai,Cf,YZff),(Xai,Cf,YZfg),(Xai,Cf,YZfh), (Xai,Cf,YZfi),(Xai,Cf,YZfj),(Xai,Cf,YZfk),(Xai,Cf,YZfl),(Xai,Cf,YZfm),(Xai,Cf,YZfn), (Xai,Cf,YZfo),(Xai,Cf,YZfp),(Xai,Cf,YZfq),(Xai,Cf,YZfr),(Xai,Cf,YZfs),(Xai,Cf, YZft),(Xai,Cf,YZfu),(Xai,Cf,YZfv),(Xai,Cf,YZfw),(Xai,Cf,YZfx),(Xai,Cf,YZfy),(Xai, Cf,YZfz),(Xai,Cf,YZga),(Xai,Cf,YZgb),(Xai,Cf,YZgc),(Xai,Cf,YZgd),(Xai,Cf,YZge), (Xai,Cf,YZgf),(Xai,Cf,YZgg),(Xai,Cf,YZgh),(Xai,Cf,YZgi),(Xai,Cf,YZgj),(Xai,Cf,YZgk), (Xai,Cf,YZgl),(Xai,Cf,YZgm),(Xai,Cf,YZgn),(Xai,Cf,YZgo),(Xai,Cf,YZgp),(Xai, Cf,YZgq),(Xai,Cf,YZgr),(Xai,Cf,YZgs),(Xai,Cf,YZgt), (Xaj,Ca,YZa),(Xaj,Ca,YZb),(Xaj,Ca,YZc),(Xaj,Ca,YZd),(Xaj,Ca,YZe),(Xaj,Ca,YZf),(Xaj, Ca,YZg),(Xaj,Ca,YZh),(Xaj,Ca,YZi),(Xaj,Ca,YZj),(Xaj,Ca,YZk),(Xaj,Ca,YZl),(Xaj, Ca,YZm),(Xaj,Ca,YZn),(Xaj,Ca,YZo),(Xaj,Ca,YZp),(Xaj,Ca,YZq),(Xaj,Ca,YZr),(Xaj, Ca,YZs),(Xaj,Ca,YZt),(Xaj,Ca,YZu),(Xaj,Ca,YZv),(Xaj,Ca,YZw),(Xaj,Ca,YZx),(Xaj, Ca,YZy),(Xaj,Ca,YZz),(Xaj,Ca,YZaa),(Xaj,Ca,YZab),(Xaj,Ca,YZac),(Xaj,Ca,YZad), (Xaj,Ca,YZae),(Xaj,Ca,YZaf),(Xaj,Ca,YZag),(Xaj,Ca,YZah),(Xaj,Ca,YZai),(Xaj,Ca, YZaj),(Xaj,Ca,YZak),(Xaj,Ca,YZal),(Xaj,Ca,YZam),(Xaj,Ca,YZan),(Xaj,Ca,YZao),(Xaj, Ca,YZap),(Xaj,Ca,YZaq),(Xaj,Ca,YZar),(Xaj,Ca,YZas),(Xaj,Ca,YZat),(Xaj,Ca,YZau), (Xaj,Ca,YZav),(Xaj,Ca,YZaw),(Xaj,Ca,YZax),(Xaj,Ca,YZay),(Xaj,Ca,YZaz),(Xaj, Ca,YZba),(Xaj,Ca,YZbb),(Xaj,Ca,YZbc),(Xaj,Ca,YZbd),(Xaj,Ca,YZbe),(Xaj,Ca,YZbf), (Xaj,Ca,YZbg),(Xaj,Ca,YZbh),(Xaj,Ca,YZbi),(Xaj,Ca,YZbj),(Xaj,Ca,YZbk),(Xaj,Ca, YZbl),(Xaj,Ca,YZbm),(Xaj,Ca,YZbn),(Xaj,Ca,YZbo),(Xaj,Ca,YZbp),(Xaj,Ca,YZbq), (Xaj,Ca,YZbr),(Xaj,Ca,YZbs),(Xaj,Ca,YZbt),(Xaj,Ca,YZbu),(Xaj,Ca,YZbv),(Xaj,Ca, YZbw),(Xaj,Ca,YZbx),(Xaj,Ca,YZby),(Xaj,Ca,YZbz),(Xaj,Ca,YZca),(Xaj,Ca,YZcb),(Xaj, Ca,YZcc),(Xaj,Ca,YZcd),(Xaj,Ca,YZce),(Xaj,Ca,YZcf),(Xaj,Ca,YZcg),(Xaj,Ca,YZch), (Xaj,Ca,YZci),(Xaj,Ca,YZcj),(Xaj,Ca,YZck),(Xaj,Ca,YZcl),(Xaj,Ca,YZcm),(Xaj, Ca,YZcn),(Xaj,Ca,YZco),(Xaj,Ca,YZcp),(Xaj,Ca,YZcq),(Xaj,Ca,YZcr),(Xaj,Ca,YZcs), (Xaj,Ca,YZct),(Xaj,Ca,YZcu),(Xaj,Ca,YZcv),(Xaj,Ca,YZcw),(Xaj,Ca,YZcx),(Xaj,Ca, YZcy),(Xaj,Ca,YZcz),(Xaj,Ca,YZda),(Xaj,Ca,YZdb),(Xaj,Ca,YZdc),(Xaj,Ca,YZdd),(Xaj, Ca,YZde),(Xaj,Ca,YZdf),(Xaj,Ca,YZdg),(Xaj,Ca,YZdh),(Xaj,Ca,YZdi),(Xaj,Ca,YZdj), (Xaj,Ca,YZdk),(Xaj,Ca,YZdl),(Xaj,Ca,YZdm),(Xaj,Ca,YZdn),(Xaj,Ca,YZdo),(Xaj, Ca,YZdp),(Xaj,Ca,YZdq),(Xaj,Ca,YZdr),(Xaj,Ca,YZds),(Xaj,Ca,Yzdt),(Xaj,Ca,YZdu), (Xaj,Ca,YZdv),(Xaj,Ca,YZdw),(Xaj,Ca,YZdx),(Xaj,Ca,YZdy),(Xaj,Ca,YZdz),(Xaj, Ca,YZea),(Xaj,Ca,YZeb),(Xaj,Ca,YZec),(Xaj,Ca,YZed),(Xaj,Ca,YZee),(Xaj,Ca,YZef), (Xaj,Ca,YZeg),(Xaj,Ca,YZeh),(Xaj,Ca,YZei),(Xaj,Ca,YZej),(Xaj,Ca,YZek),(Xaj,Ca, YZel),(Xaj,Ca,YZem),(Xaj,Ca,YZen),(Xaj,Ca,YZeo),(Xaj,Ca,YZep),(Xaj,Ca,YZeq),(Xaj, Ca,YZer),(Xaj,Ca,YZes),(Xaj,Ca,YZet),(Xaj,Ca,YZeu),(Xaj,Ca,YZev),(Xaj,Ca,YZew), (Xaj,Ca,YZex),(Xaj,Ca,YZey),(Xaj,Ca,YZez),(Xaj,Ca,YZfa),(Xaj,Ca,YZfb),(Xaj, Ca,YZfc),(Xaj,Ca,YZfd),(Xaj,Ca,YZfe),(Xaj,Ca,YZff),(Xaj,Ca,YZfg),(Xaj,Ca,YZfh), (Xaj,Ca,YZfi),(Xaj,Ca,YZfj),(Xaj,Ca,YZfk),(Xaj,Ca,YZfl),(Xaj,Ca,YZfm),(Xaj,Ca,YZfn), (Xaj,Ca,YZfo),(Xaj,Ca,YZfp),(Xaj,Ca,YZfq),(Xaj,Ca,YZfr),(Xaj,Ca,YZfs),(Xaj, Ca,YZft),(Xaj,Ca,YZfu),(Xaj,Ca,YZfv),(Xaj,Ca,YZfw),(Xaj,Ca,YZfx),(Xaj,Ca,YZfy), (Xaj,Ca,YZfz),(Xaj,Ca,YZga),(Xaj,Ca,YZgb),(Xaj,Ca,YZgc),(Xaj,Ca,YZgd),(Xaj,Ca, YZge),(Xaj,Ca,YZgf),(Xaj,Ca,YZgg),(Xaj,Ca,YZgh),(Xaj,Ca,YZgi),(Xaj,Ca,YZgj),(Xaj, Ca,YZgk),(Xaj,Ca,YZgl),(Xaj,Ca,YZgm),(Xaj,Ca,YZgn),(Xaj,Ca,YZgo),(Xaj,Ca,YZgp), (Xaj,Ca,YZgq),(Xaj,Ca,YZgr),(Xaj,Ca,YZgs),(Xaj,Ca,YZgt),(Xaj,Cb,YZa),(Xaj, Cb,YZb),(Xaj,Cb,YZc),(Xaj,Cb,YZd),(Xaj,Cb,YZe),(Xaj,Cb,YZf),(Xaj,Cb,YZg),(Xaj, Cb,YZh),(Xaj,Cb,YZi),(Xaj,Cb,YZj),(Xaj,Cb,YZk),(Xaj,Cb,YZl),(Xaj,Cb,YZm),(Xaj, Cb,YZn),(Xaj,Cb,YZo),(Xaj,Cb,YZp),(Xaj,Cb,YZq),(Xaj,Cb,YZr),(Xaj,Cb,YZs),(Xaj, Cb,YZt),(Xaj,Cb,YZu),(Xaj,Cb,YZv),(Xaj,Cb,YZw),(Xaj,Cb,YZx),(Xaj,Cb,YZy),(Xaj, Cb,YZz),(Xaj,Cb,YZaa),(Xaj,Cb,YZab),(Xaj,Cb,YZac),(Xaj,Cb,YZad),(Xaj,Cb,YZae), (Xaj,Cb,YZaf),(Xaj,Cb,YZag),(Xaj,Cb,YZah),(Xaj,Cb,YZai),(Xaj,Cb,YZaj),(Xaj,Cb, YZak),(Xaj,Cb,YZal),(Xaj,Cb,YZam),(Xaj,Cb,YZan),(Xaj,Cb,YZao),(Xaj,Cb,YZap),(Xaj, Cb,YZaq),(Xaj,Cb,YZar),(Xaj,Cb,YZas),(Xaj,Cb,YZat),(Xaj,Cb,YZau),(Xaj,Cb,YZav), (Xaj,Cb,YZaw),(Xaj,Cb,YZax),(Xaj,Cb,YZay),(Xaj,Cb,YZaz),(Xaj,Cb,YZba),(Xaj, Cb,YZbb),(Xaj,Cb,YZbc),(Xaj,Cb,YZbd),(Xaj,Cb,YZbe),(Xaj,Cb,YZbf),(Xaj,Cb,YZbg), (Xaj,Cb,YZbh),(Xaj,Cb,YZbi),(Xaj,Cb,YZbj),(Xaj,Cb,YZbk),(Xaj,Cb,YZbl),(Xaj, Cb,YZbm),(Xaj,Cb,YZbn),(Xaj,Cb,YZbo),(Xaj,Cb,YZbp),(Xaj,Cb,YZbq),(Xaj,Cb,YZbr), (Xaj,Cb,YZbs),(Xaj,Cb,YZbt),(Xaj,Cb,YZbu),(Xaj,Cb,YZbv),(Xaj,Cb,YZbw),(Xaj, Cb,YZbx),(Xaj,Cb,YZby),(Xaj,Cb,YZbz),(Xaj,Cb,YZca),(Xaj,Cb,YZcb),(Xaj,Cb,YZcc), (Xaj,Cb,YZcd),(Xaj,Cb,YZce),(Xaj,Cb,YZcf),(Xaj,Cb,YZcg),(Xaj,Cb,YZch),(Xaj, Cb,YZci),(Xaj,Cb,YZcj),(Xaj,Cb,YZck),(Xaj,Cb,YZcl),(Xaj,Cb,YZcm),(Xaj,Cb,YZcn), (Xaj,Cb,YZco),(Xaj,Cb,YZcp),(Xaj,Cb,YZcq),(Xaj,Cb,YZcr),(Xaj,Cb,YZcs),(Xaj, Cb,YZct),(Xaj,Cb,YZcu),(Xaj,Cb,YZcv),(Xaj,Cb,YZcw),(Xaj,Cb,YZcx),(Xaj,Cb,YZcy), (Xaj,Cb,YZcz),(Xaj,Cb,YZda),(Xaj,Cb,YZdb),(Xaj,Cb,YZdc),(Xaj,Cb,YZdd),(Xaj, Cb,YZde),(Xaj,Cb,YZdf),(Xaj,Cb,YZdg),(Xaj,Cb,YZdh),(Xaj,Cb,YZdi),(Xaj,Cb,YZdj), (Xaj,Cb,YZdk),(Xaj,Cb,YZdl),(Xaj,Cb,YZdm),(Xaj,Cb,YZdn),(Xaj,Cb,YZdo),(Xaj, Cb,YZdp),(Xaj,Cb,YZdq),(Xaj,Cb,YZdr),(Xaj,Cb,YZds),(Xaj,Cb,Yzdt),(Xaj,Cb,YZdu), (Xaj,Cb,YZdv),(Xaj,Cb,YZdw),(Xaj,Cb,YZdx),(Xaj,Cb,YZdy),(Xaj,Cb,YZdz),(Xaj, Cb,YZea),(Xaj,Cb,YZeb),(Xaj,Cb,YZec),(Xaj,Cb,YZed),(Xaj,Cb,YZee),(Xaj,Cb,YZef), (Xaj,Cb,YZeg),(Xaj,Cb,YZeh),(Xaj,Cb,YZei),(Xaj,Cb,YZej),(Xaj,Cb,YZek),(Xaj,Cb, YZel),(Xaj,Cb,YZem),(Xaj,Cb,YZen),(Xaj,Cb,YZeo),(Xaj,Cb,YZep),(Xaj,Cb,YZeq), (Xaj,Cb,YZer),(Xaj,Cb,YZes),(Xaj,Cb,YZet),(Xaj,Cb,YZeu),(Xaj,Cb,YZev),(Xaj,Cb, YZew),(Xaj,Cb,YZex),(Xaj,Cb,YZey),(Xaj,Cb,YZez),(Xaj,Cb,YZfa),(Xaj,Cb,YZfb),(Xaj, Cb,YZfc),(Xaj,Cb,YZfd),(Xaj,Cb,YZfe),(Xaj,Cb,YZff),(Xaj,Cb,YZfg),(Xaj,Cb,YZfh), (Xaj,Cb,YZfi),(Xaj,Cb,YZfj),(Xaj,Cb,YZfk),(Xaj,Cb,YZfl),(Xaj,Cb,YZfm),(Xaj, Cb,YZfn),(Xaj,Cb,YZfo),(Xaj,Cb,YZfp),(Xaj,Cb,YZfq),(Xaj,Cb,YZfr),(Xaj,Cb,YZfs), (Xaj,Cb,YZft),(Xaj,Cb,YZfu),(Xaj,Cb,YZfv),(Xaj,Cb,YZfw),(Xaj,Cb,YZfx),(Xaj,Cb, YZfy),(Xaj,Cb,YZfz),(Xaj,Cb,YZga),(Xaj,Cb,YZgb),(Xaj,Cb,YZgc),(Xaj,Cb,YZgd),(Xaj, Cb,YZge),(Xaj,Cb,YZgf),(Xaj,Cb,YZgg),(Xaj,Cb,YZgh),(Xaj,Cb,YZgi),(Xaj,Cb, YZgj),(Xaj,Cb,YZgk),(Xaj,Cb,YZgl),(Xaj,Cb,YZgm),(Xaj,Cb,YZgn),(Xaj,Cb,YZgo),(Xaj, Cb,YZgp),(Xaj,Cb,YZgq),(Xaj,Cb,YZgr),(Xaj,Cb,YZgs),(Xaj,Cb,YZgt),(Xaj,Cc, YZa),(Xaj,Cc,YZb),(Xaj,Cc,YZc),(Xaj,Cc,YZd),(Xaj,Cc,YZe),(Xaj,Cc,YZf),(Xaj,Cc, YZg),(Xaj,Cc,YZh),(Xaj,Cc,YZi),(Xaj,Cc,YZj),(Xaj,Cc,YZk),(Xaj,Cc,YZl),(Xaj,Cc,YZm), (Xaj,Cc,YZn),(Xaj,Cc,YZo),(Xaj,Cc,YZp),(Xaj,Cc,YZq),(Xaj,Cc,YZr),(Xaj,Cc,YZs), (Xaj,Cc,YZt),(Xaj,Cc,YZu),(Xaj,Cc,YZv),(Xaj,Cc,YZw),(Xaj,Cc,YZx),(Xaj,Cc,YZy), (Xaj,Cc,YZz),(Xaj,Cc,YZaa),(Xaj,Cc,YZab),(Xaj,Cc,YZac),(Xaj,Cc,YZad),(Xaj, Cc,YZae),(Xaj,Cc,YZaf),(Xaj,Cc,YZag),(Xaj,Cc,YZah),(Xaj,Cc,YZai),(Xaj,Cc,YZaj), (Xaj,Cc,YZak),(Xaj,Cc,YZal),(Xaj,Cc,YZam),(Xaj,Cc,YZan),(Xaj,Cc,YZao),(Xaj,Cc, YZap),(Xaj,Cc,YZaq),(Xaj,Cc,YZar),(Xaj,Cc,YZas),(Xaj,Cc,YZat),(Xaj,Cc,YZau),(Xaj, Cc,YZav),(Xaj,Cc,YZaw),(Xaj,Cc,YZax),(Xaj,Cc,YZay),(Xaj,Cc,YZaz),(Xaj,Cc,YZba), (Xaj,Cc,YZbb),(Xaj,Cc,YZbc),(Xaj,Cc,YZbd),(Xaj,Cc,YZbe),(Xaj,Cc,YZbf),(Xaj, Cc,YZbg),(Xaj,Cc,YZbh),(Xaj,Cc,YZbi),(Xaj,Cc,YZbj),(Xaj,Cc,YZbk),(Xaj,Cc,YZbl), (Xaj,Cc,YZbm),(Xaj,Cc,YZbn),(Xaj,Cc,YZbo),(Xaj,Cc,YZbp),(Xaj,Cc,YZbq),(Xaj, Cc,YZbr),(Xaj,Cc,YZbs),(Xaj,Cc,YZbt),(Xaj,Cc,YZbu),(Xaj,Cc,YZbv),(Xaj,Cc,YZbw), (Xaj,Cc,YZbx),(Xaj,Cc,YZby),(Xaj,Cc,YZbz),(Xaj,Cc,YZca),(Xaj,Cc,YZcb),(Xaj,Cc, YZcc),(Xaj,Cc,YZcd),(Xaj,Cc,YZce),(Xaj,Cc,YZcf),(Xaj,Cc,YZcg),(Xaj,Cc,YZch),(Xaj, Cc,YZci),(Xaj,Cc,YZcj),(Xaj,Cc,YZck),(Xaj,Cc,YZcl),(Xaj,Cc,YZcm),(Xaj,Cc,YZcn), (Xaj,Cc,YZco),(Xaj,Cc,YZcp),(Xaj,Cc,YZcq),(Xaj,Cc,YZcr),(Xaj,Cc,YZcs),(Xaj, Cc,YZct),(Xaj,Cc,YZcu),(Xaj,Cc,YZcv),(Xaj,Cc,YZcw),(Xaj,Cc,YZcx),(Xaj,Cc,YZcy), (Xaj,Cc,YZcz),(Xaj,Cc,YZda),(Xaj,Cc,YZdb),(Xaj,Cc,YZdc),(Xaj,Cc,YZdd),(Xaj, Cc,YZde),(Xaj,Cc,YZdf),(Xaj,Cc,YZdg),(Xaj,Cc,YZdh),(Xaj,Cc,YZdi),(Xaj,Cc,YZdj), (Xaj,Cc,YZdk),(Xaj,Cc,YZdl),(Xaj,Cc,YZdm),(Xaj,Cc,YZdn),(Xaj,Cc,YZdo),(Xaj,Cc, YZdp),(Xaj,Cc,YZdq),(Xaj,Cc,YZdr),(Xaj,Cc,YZds),(Xaj,Cc,Yzdt),(Xaj,Cc,YZdu),(Xaj, Cc,YZdv),(Xaj,Cc,YZdw),(Xaj,Cc,YZdx),(Xaj,Cc,YZdy),(Xaj,Cc,YZdz),(Xaj,Cc, YZea),(Xaj,Cc,YZeb),(Xaj,Cc,YZec),(Xaj,Cc,YZed),(Xaj,Cc,YZee),(Xaj,Cc,YZef),(Xaj, Cc,YZeg),(Xaj,Cc,YZeh),(Xaj,Cc,YZei),(Xaj,Cc,YZej),(Xaj,Cc,YZek),(Xaj,Cc,YZel), (Xaj,Cc,YZem),(Xaj,Cc,YZen),(Xaj,Cc,YZeo),(Xaj,Cc,YZep),(Xaj,Cc,YZeq),(Xaj, Cc,YZer),(Xaj,Cc,YZes),(Xaj,Cc,YZet),(Xaj,Cc,YZeu),(Xaj,Cc,YZev),(Xaj,Cc,YZew), (Xaj,Cc,YZex),(Xaj,Cc,YZey),(Xaj,Cc,YZez),(Xaj,Cc,YZfa),(Xaj,Cc,YZfb),(Xaj,Cc, YZfc),(Xaj,Cc,YZfd),(Xaj,Cc,YZfe),(Xaj,Cc,YZff),(Xaj,Cc,YZfg),(Xaj,Cc,YZfh),(Xaj, Cc,YZfi),(Xaj,Cc,YZfj),(Xaj,Cc,YZfk),(Xaj,Cc,YZfl),(Xaj,Cc,YZfm),(Xaj,Cc,YZfn),(Xaj, Cc,YZfo),(Xaj,Cc,YZfp),(Xaj,Cc,YZfq),(Xaj,Cc,YZfr),(Xaj,Cc,YZfs),(Xaj,Cc,YZft), (Xaj,Cc,YZfu),(Xaj,Cc,YZfv),(Xaj,Cc,YZfw),(Xaj,Cc,YZfx),(Xaj,Cc,YZfy),(Xaj,Cc, YZfz),(Xaj,Cc,YZga),(Xaj,Cc,YZgb),(Xaj,Cc,YZgc),(Xaj,Cc,YZgd),(Xaj,Cc,YZge), (Xaj,Cc,YZgf),(Xaj,Cc,YZgg),(Xaj,Cc,YZgh),(Xaj,Cc,YZgi),(Xaj,Cc,YZgj),(Xaj,Cc, YZgk),(Xaj,Cc,YZgl),(Xaj,Cc,YZgm),(Xaj,Cc,YZgn),(Xaj,Cc,YZgo),(Xaj,Cc,YZgp),(Xaj, Cc,YZgq),(Xaj,Cc,YZgr),(Xaj,Cc,YZgs),(Xaj,Cc,YZgt),(Xaj,Cd,YZa),(Xaj,Cd,YZb), (Xaj,Cd,YZc),(Xaj,Cd,YZd),(Xaj,Cd,YZe),(Xaj,Cd,YZf),(Xaj,Cd,YZg),(Xaj,Cd,YZh), (Xaj,Cd,YZi),(Xaj,Cd,YZj),(Xaj,Cd,YZk),(Xaj,Cd,YZl),(Xaj,Cd,YZm),(Xaj,Cd,YZn), (Xaj,Cd,YZo),(Xaj,Cd,YZp),(Xaj,Cd,YZq),(Xaj,Cd,YZr),(Xaj,Cd,YZs),(Xaj,Cd,YZt), Xaj,Cd,YZu),(Xaj,Cd,YZv),(Xaj,Cd,YZw),(Xaj,Cd,YZx),(Xaj,Cd,YZy),(Xaj,Cd,YZz), (Xaj,Cd,YZaa),(Xaj,Cd,YZab),(Xaj,Cd,YZac),(Xaj,Cd,YZad),(Xaj,Cd,YZae),(Xaj, Cd,YZaf),(Xaj,Cd,YZag),(Xaj,Cd,YZah),(Xaj,Cd,YZai),(Xaj,Cd,YZaj),(Xaj,Cd,YZak), (Xaj,Cd,YZal),(Xaj,Cd,YZam),(Xaj,Cd,YZan),(Xaj,Cd,YZao),(Xaj,Cd,YZap),(Xaj,Cd, YZaq),(Xaj,Cd,YZar),(Xaj,Cd,YZas),(Xaj,Cd,YZat),(Xaj,Cd,YZau),(Xaj,Cd,YZav),(Xaj, Cd,YZaw),(Xaj,Cd,YZax),(Xaj,Cd,YZay),(Xaj,Cd,YZaz),(Xaj,Cd,YZba),(Xaj,Cd, YZbb),(Xaj,Cd,YZbc),(Xaj,Cd,YZbd),(Xaj,Cd,YZbe),(Xaj,Cd,YZbf),(Xaj,Cd,YZbg),(Xaj, Cd,YZbh),(Xaj,Cd,YZbi),(Xaj,Cd,YZbj),(Xaj,Cd,YZbk),(Xaj,Cd,YZbl),(Xaj,Cd,YZbm), (Xaj,Cd,YZbn),(Xaj,Cd,YZbo),(Xaj,Cd,YZbp),(Xaj,Cd,YZbq),(Xaj,Cd,YZbr),(Xaj, Cd,YZbs),(Xaj,Cd,YZbt),(Xaj,Cd,YZbu),(Xaj,Cd,YZbv),(Xaj,Cd,YZbw),(Xaj,Cd, YZbx),(Xaj,Cd,YZby),(Xaj,Cd,YZbz),(Xaj,Cd,YZca),(Xaj,Cd,YZcb),(Xaj,Cd,YZcc),(Xaj, Cd,YZcd),(Xaj,Cd,YZce),(Xaj,Cd,YZcf),(Xaj,Cd,YZcg),(Xaj,Cd,YZch),(Xaj,Cd, YZci),(Xaj,Cd,YZcj),(Xaj,Cd,YZck),(Xaj,Cd,YZcl),(Xaj,Cd,YZcm),(Xaj,Cd,YZcn),(Xaj, Cd,YZco),(Xaj,Cd,YZcp),(Xaj,Cd,YZcq),(Xaj,Cd,YZcr),(Xaj,Cd,YZcs),(Xaj,Cd, YZct),(Xaj,Cd,YZcu),(Xaj,Cd,YZcv),(Xaj,Cd,YZcw),(Xaj,Cd,YZcx),(Xaj,Cd,YZcy),(Xaj, Cd,YZcz),(Xaj,Cd,YZda),(Xaj,Cd,YZdb),(Xaj,Cd,YZdc),(Xaj,Cd,YZdd),(Xaj,Cd, YZde),(Xaj,Cd,YZdf),(Xaj,Cd,YZdg),(Xaj,Cd,YZdh),(Xaj,Cd,YZdi),(Xaj,Cd,YZdj),(Xaj, Cd,YZdk),(Xaj,Cd,YZdl),(Xaj,Cd,YZdm),(Xaj,Cd,YZdn),(Xaj,Cd,YZdo),(Xaj,Cd, YZdp),(Xaj,Cd,YZdq),(Xaj,Cd,YZdr),(Xaj,Cd,YZds),(Xaj,Cd,Yzdt),(Xaj,Cd,YZdu),(Xaj, Cd,YZdv),(Xaj,Cd,YZdw),(Xaj,Cd,YZdx),(Xaj,Cd,YZdy),(Xaj,Cd,YZdz),(Xaj,Cd, YZea),(Xaj,Cd,YZeb),(Xaj,Cd,YZec),(Xaj,Cd,YZed),(Xaj,Cd,YZee),(Xaj,Cd,YZef),(Xaj, Cd,YZeg),(Xaj,Cd,YZeh),(Xaj,Cd,YZei),(Xaj,Cd,YZej),(Xaj,Cd,YZek),(Xaj,Cd,YZel), (Xaj,Cd,YZem),(Xaj,Cd,YZen),(Xaj,Cd,YZeo),(Xaj,Cd,YZep),(Xaj,Cd,YZeq),(Xaj, Cd,YZer),(Xaj,Cd,YZes),(Xaj,Cd,YZet),(Xaj,Cd,YZeu),(Xaj,Cd,YZev),(Xaj,Cd,YZew), (Xaj,Cd,YZex),(Xaj,Cd,YZey),(Xaj,Cd,YZez),(Xaj,Cd,YZfa),(Xaj,Cd,YZfb),(Xaj, Cd,YZfc),(Xaj,Cd,YZfd),(Xaj,Cd,YZfe),(Xaj,Cd,YZff),(Xaj,Cd,YZfg),(Xaj,Cd,YZfh), (Xaj,Cd,YZfi),(Xaj,Cd,YZfj),(Xaj,Cd,YZfk),(Xaj,Cd,YZfl),(Xaj,Cd,YZfm),(Xaj,Cd,YZfn), (Xaj,Cd,YZfo),(Xaj,Cd,YZfp),(Xaj,Cd,YZfq),(Xaj,Cd,YZfr),(Xaj,Cd,YZfs),(Xaj, Cd,YZft),(Xaj,Cd,YZfu),(Xaj,Cd,YZfv),(Xaj,Cd,YZfw),(Xaj,Cd,YZfx),(Xaj,Cd,YZfy), (Xaj,Cd,YZfz),(Xaj,Cd,YZga),(Xaj,Cd,YZgb),(Xaj,Cd,YZgc),(Xaj,Cd,YZgd),(Xaj,Cd, YZge),(Xaj,Cd,YZgf),(Xaj,Cd,YZgg),(Xaj,Cd,YZgh),(Xaj,Cd,YZgi),(Xaj,Cd,YZgj),(Xaj, Cd,YZgk),(Xaj,Cd,YZgl),(Xaj,Cd,YZgm),(Xaj,Cd,YZgn),(Xaj,Cd,YZgo),(Xaj,Cd, YZgp),(Xaj,Cd,YZgq),(Xaj,Cd,YZgr),(Xaj,Cd,YZgs),(Xaj,Cd,YZgt),(Xaj,Ce,YZa),(Xaj, Ce,YZb),(Xaj,Ce,YZc),(Xaj,Ce,YZd),(Xaj,Ce,YZe),(Xaj,Ce,YZf),(Xaj,Ce,YZg),(Xaj, Ce,YZh),(Xaj,Ce,YZi),(Xaj,Ce,YZj),(Xaj,Ce,YZk),(Xaj,Ce,YZl),(Xaj,Ce,YZm),(Xaj, Ce,YZn),(Xaj,Ce,YZo),(Xaj,Ce,YZp),(Xaj,Ce,YZq),(Xaj,Ce,YZr),(Xaj,Ce,YZs),(Xaj, Ce,YZt),(Xaj,Ce,YZu),(Xaj,Ce,YZv),(Xaj,Ce,YZw),(Xaj,Ce,YZx),(Xaj,Ce,YZy),(Xaj, Ce,YZz),(Xaj,Ce,YZaa),(Xaj,Ce,YZab),(Xaj,Ce,YZac),(Xaj,Ce,YZad),(Xaj,Ce,YZae), (Xaj,Ce,YZaf),(Xaj,Ce,YZag),(Xaj,Ce,YZah),(Xaj,Ce,YZai),(Xaj,Ce,YZaj),(Xaj,Ce,YZak), (Xaj,Ce,YZal),(Xaj,Ce,YZam),(Xaj,Ce,YZan),(Xaj,Ce,YZao),(Xaj,Ce,YZap),(Xaj, Ce,YZaq),(Xaj,Ce,YZar),(Xaj,Ce,YZas),(Xaj,Ce,YZat),(Xaj,Ce,YZau),(Xaj,Ce,YZav), (Xaj,Ce,YZaw),(Xaj,Ce,YZax),(Xaj,Ce,YZay),(Xaj,Ce,YZaz),(Xaj,Ce,YZba),(Xaj,Ce, YZbb),(Xaj,Ce,YZbc),(Xaj,Ce,YZbd),(Xaj,Ce,YZbe),(Xaj,Ce,YZbf),(Xaj,Ce,YZbg), (Xaj,Ce,YZbh),(Xaj,Ce,YZbi),(Xaj,Ce,YZbj),(Xaj,Ce,YZbk),(Xaj,Ce,YZbl),(Xaj,Ce,YZbm), (Xaj,Ce,YZbn),(Xaj,Ce,YZbo),(Xaj,Ce,YZbp),(Xaj,Ce,YZbq),(Xaj,Ce,YZbr),(Xaj, Ce,YZbs),(Xaj,Ce,YZbt),(Xaj,Ce,YZbu),(Xaj,Ce,YZbv),(Xaj,Ce,YZbw),(Xaj,Ce,YZbx), (Xaj,Ce,YZby),(Xaj,Ce,YZbz),(Xaj,Ce,YZca),(Xaj,Ce,YZcb),(Xaj,Ce,YZcc),(Xaj, Ce,YZcd),(Xaj,Ce,YZce),(Xaj,Ce,YZcf),(Xaj,Ce,YZcg),(Xaj,Ce,YZch),(Xaj,Ce,YZci), (Xaj,Ce,YZcj),(Xaj,Ce,YZck),(Xaj,Ce,YZcl),(Xaj,Ce,YZcm),(Xaj,Ce,YZcn),(Xaj,Ce, YZco),(Xaj,Ce,YZcp),(Xaj,Ce,YZcq),(Xaj,Ce,YZcr),(Xaj,Ce,YZcs),(Xaj,Ce,YZct),(Xaj, Ce,YZcu),(Xaj,Ce,YZcv),(Xaj,Ce,YZcw),(Xaj,Ce,YZcx),(Xaj,Ce,YZcy),(Xaj,Ce,YZcz), (Xaj,Ce,YZda),(Xaj,Ce,YZdb),(Xaj,Ce,YZdc),(Xaj,Ce,YZdd),(Xaj,Ce,YZde),(Xaj, Ce,YZdf),(Xaj,Ce,YZdg),(Xaj,Ce,YZdh),(Xaj,Ce,YZdi),(Xaj,Ce,YZdj),(Xaj,Ce,YZdk), (Xaj,Ce,YZdl),(Xaj,Ce,YZdm),(Xaj,Ce,YZdn),(Xaj,Ce,YZdo),(Xaj,Ce,YZdp),(Xaj, Ce,YZdq),(Xaj,Ce,YZdr),(Xaj,Ce,YZds),(Xaj,Ce,Yzdt),(Xaj,Ce,YZdu),(Xaj,Ce,YZdv), (Xaj,Ce,YZdw),(Xaj,Ce,YZdx),(Xaj,Ce,YZdy),(Xaj,Ce,YZdz),(Xaj,Ce,YZea),(Xaj,Ce, YZeb),(Xaj,Ce,YZec),(Xaj,Ce,YZed),(Xaj,Ce,YZee),(Xaj,Ce,YZef),(Xaj,Ce,YZeg),(Xaj, Ce,YZeh),(Xaj,Ce,YZei),(Xaj,Ce,YZej),(Xaj,Ce,YZek),(Xaj,Ce,YZel),(Xaj,Ce,YZem), (Xaj,Ce,YZen),(Xaj,Ce,YZeo),(Xaj,Ce,YZep),(Xaj,Ce,YZeq),(Xaj,Ce,YZer),(Xaj, Ce,YZes),(Xaj,Ce,YZet),(Xaj,Ce,YZeu),(Xaj,Ce,YZev),(Xaj,Ce,YZew),(Xaj,Ce,YZex), (Xaj,Ce,YZey),(Xaj,Ce,YZez),(Xaj,Ce,YZfa),(Xaj,Ce,YZfb),(Xaj,Ce,YZfc),(Xaj,Ce, YZfd),(Xaj,Ce,YZfe),(Xaj,Ce,YZff),(Xaj,Ce,YZfg),(Xaj,Ce,YZfh),(Xaj,Ce,YZfi),(Xaj, Ce,YZfj),(Xaj,Ce,YZfk),(Xaj,Ce,YZfl),(Xaj,Ce,YZfm),(Xaj,Ce,YZfn),(Xaj,Ce,YZfo), (Xaj,Ce,YZp),(Xaj,Ce,YZfq),(Xaj,Ce,YZfr),(Xaj,Ce,YZfs),(Xaj,Ce,YZft),(Xaj,Ce,YZfu), (Xaj,Ce,YZfv),(Xaj,Ce,YZfw),(Xaj,Ce,YZfx),(Xaj,Ce,YZfy),(Xaj,Ce,YZfz),(Xaj,Ce, YZga),(Xaj,Ce,YZgb),(Xaj,Ce,YZgc),(Xaj,Ce,YZgd),(Xaj,Ce,YZge),(Xaj,Ce,YZgf), (Xaj,Ce,YZgg),(Xaj,Ce,YZgh),(Xaj,Ce,YZgi),(Xaj,Ce,YZgj),(Xaj,Ce,YZgk),(Xaj,Ce, YZgl),(Xaj,Ce,YZgm),(Xaj,Ce,YZgn),(Xaj,Ce,YZgo),(Xaj,Ce,YZgp),(Xaj,Ce,YZgq),(Xaj, Ce,YZgr),(Xaj,Ce,YZgs),(Xaj,Ce,YZgt),(Xaj,Cf,YZa),(Xaj,Cf,YZb),(Xaj,Cf,YZc), (Xaj,Cf,YZd),(Xaj,Cf,YZe),(Xaj,Cf,YZf),(Xaj,Cf,YZg),(Xaj,Cf,YZh),(Xaj,Cf,YZi),(Xaj, Cf,YZj),(Xaj,Cf,YZk),(Xaj,Cf,YZl),(Xaj,Cf,YZm),(Xaj,Cf,YZn),(Xaj,Cf,YZo),(Xaj, Cf,YZp),(Xaj,Cf,YZq),(Xaj,Cf,YZr),(Xaj,Cf,YZs),(Xaj,Cf,YZt),(Xaj,Cf,YZu),(Xaj, Cf,YZv),(Xaj,Cf,YZw),(Xaj,Cf,YZx),(Xaj,Cf,YZy),(Xaj,Cf,YZz),(Xaj,Cf,YZaa),(Xaj, Cf,YZab),(Xaj,Cf,YZac),(Xaj,Cf,YZad),(Xaj,Cf,YZae),(Xaj,Cf,YZaf),(Xaj,Cf,YZag),(Xaj, Cf,YZah),(Xaj,Cf,YZai),(Xaj,Cf,YZaj),(Xaj,Cf,YZak),(Xaj,Cf,YZal),(Xaj,Cf,YZam), (Xaj,Cf,YZan),(Xaj,Cf,YZao),(Xaj,Cf,YZap),(Xaj,Cf,YZaq),(Xaj,Cf,YZar),(Xaj,Cf, YZas),(Xaj,Cf,YZat),(Xaj,Cf,YZau),(Xaj,Cf,YZav),(Xaj,Cf,YZaw),(Xaj,Cf,YZax),(Xaj, Cf,YZay),(Xaj,Cf,YZaz),(Xaj,Cf,YZba),(Xaj,Cf,YZbb),(Xaj,Cf,YZbc),(Xaj,Cf,YZbd), (Xaj,Cf,YZbe),(Xaj,Cf,YZbf),(Xaj,Cf,YZbg),(Xaj,Cf,YZbh),(Xaj,Cf,YZbi),(Xaj,Cf, YZbj),(Xaj,Cf,YZbk),(Xaj,Cf,YZbl),(Xaj,Cf,YZbm),(Xaj,Cf,YZbn),(Xaj,Cf,YZbo),(Xaj, Cf,YZbp),(Xaj,Cf,YZbq),(Xaj,Cf,YZbr),(Xaj,Cf,YZbs),(Xaj,Cf,YZbt),(Xaj,Cf,YZbu), (Xaj,Cf,YZbv),(Xaj,Cf,YZbw),(Xaj,Cf,YZbx),(Xaj,Cf,YZby),(Xaj,Cf,YZbz),(Xaj,Cf, YZca),(Xaj,Cf,YZcb),(Xaj,Cf,YZcc),(Xaj,Cf,YZcd),(Xaj,Cf,YZce),(Xaj,Cf,YZcf),(Xaj, Cf,YZcg),(Xaj,Cf,YZch),(Xaj,Cf,YZci),(Xaj,Cf,YZcj),(Xaj,Cf,YZck),(Xaj,Cf,YZcl), (Xaj,Cf,YZcm),(Xaj,Cf,YZcn),(Xaj,Cf,YZco),(Xaj,Cf,YZcp),(Xaj,Cf,YZcq),(Xaj,Cf, YZcr),(Xaj,Cf,YZcs),(Xaj,Cf,YZct),(Xaj,Cf,YZcu),(Xaj,Cf,YZcv),(Xaj,Cf,YZcw),(Xaj, Cf,YZcx),(Xaj,Cf,YZcy),(Xaj,Cf,YZcz),(Xaj,Cf,YZda),(Xaj,Cf,YZdb),(Xaj,Cf,YZdc), (Xaj,Cf,YZdd),(Xaj,Cf,YZde),(Xaj,Cf,YZdf),(Xaj,Cf,YZdg),(Xaj,Cf,YZdh),(Xaj,Cf, YZdi),(Xaj,Cf,YZdj),(Xaj,Cf,YZdk),(Xaj,Cf,YZdl),(Xaj,Cf,YZdm),(Xaj,Cf,YZdn),(Xaj, Cf,YZdo),(Xaj,Cf,YZdp),(Xaj,Cf,YZdq),(Xaj,Cf,YZdr),(Xaj,Cf,YZds),(Xaj,Cf,Yzdt), (Xaj,Cf,YZdu),(Xaj,Cf,YZdv),(Xaj,Cf,YZdw),(Xaj,Cf,YZdx),(Xaj,Cf,YZdy),(Xaj,Cf, YZdz),(Xaj,Cf,YZea),(Xaj,Cf,YZeb),(Xaj,Cf,YZec),(Xaj,Cf,YZed),(Xaj,Cf,YZee),(Xaj, Cf,YZef),(Xaj,Cf,YZeg),(Xaj,Cf,YZeh),(Xaj,Cf,YZei),(Xaj,Cf,YZej),(Xaj,Cf,YZek), (Xaj,Cf,YZel),(Xaj,Cf,YZem),(Xaj,Cf,YZen),(Xaj,Cf,YZeo),(Xaj,Cf,YZep),(Xaj,Cf,YZeq), (Xaj,Cf,YZer),(Xaj,Cf,YZes),(Xaj,Cf,YZet),(Xaj,Cf,YZeu),(Xaj,Cf,YZev),(Xaj, Cf,YZew),(Xaj,Cf,YZex),(Xaj,Cf,YZey),(Xaj,Cf,YZez),(Xaj,Cf,YZfa),(Xaj,Cf,YZfb), (Xaj,Cf,YZfc),(Xaj,Cf,YZfd),(Xaj,Cf,YZfe),(Xaj,Cf,YZff),(Xaj,Cf,YZfg),(Xaj,Cf,YZfh), (Xaj,Cf,YZfi),(Xaj,Cf,YZfj),(Xaj,Cf,YZfk),(Xaj,Cf,YZfl),(Xaj,Cf,YZfm),(Xaj,Cf,YZfn), (Xaj,Cf,YZfo),(Xaj,Cf,YZfp),(Xaj,Cf,YZfq),(Xaj,Cf,YZfr),(Xaj,Cf,YZfs),(Xaj,Cf, YZft),(Xaj,Cf,YZfu),(Xaj,Cf,YZfv),(Xaj,Cf,YZfw),(Xaj,Cf,YZfx),(Xaj,Cf,YZfy),(Xaj, Cf,YZfz),(Xaj,Cf,YZga),(Xaj,Cf,YZgb),(Xaj,Cf,YZgc),(Xaj,Cf,YZgd),(Xaj,Cf,YZge), (Xaj,Cf,YZgf),(Xaj,Cf,YZgg),(Xaj,Cf,YZgh),(Xaj,Cf,YZgi),(Xaj,Cf,YZgj),(Xaj,Cf,YZgk), (Xaj,Cf,YZgl),(Xaj,Cf,YZgm),(Xaj,Cf,YZgn),(Xaj,Cf,YZgo),(Xaj,Cf,YZgp),(Xaj, Cf,YZgq),(Xaj,Cf,YZgr),(Xaj,Cf,YZgs),(Xaj,Cf,YZgt), (Xaj,Ca,YZa),(Xak,Ca,YZb),(Xak,Ca,YZc),(Xak,Ca,YZd),(Xak,Ca,YZe),(Xak,Ca,YZf), (Xak,Ca,YZg),(Xak,Ca,YZh),(Xak,Ca,YZi),(Xak,Ca,YZj),(Xak,Ca,YZk),(Xak,Ca,YZl), (Xak,Ca,YZm),(Xak,Ca,YZn),(Xak,Ca,YZo),(Xak,Ca,YZp),(Xak,Ca,YZq),(Xak,Ca, YZr),(Xak,Ca,YZs),(Xak,Ca,YZt),(Xak,Ca,YZu),(Xak,Ca,YZv),(Xak,Ca,YZw),(Xak, Ca,YZx),(Xak,Ca,YZy),(Xak,Ca,YZz),(Xak,Ca,YZaa),(Xak,Ca,YZab),(Xak,Ca,YZac), (Xak,Ca,YZad),(Xak,Ca,YZae),(Xak,Ca,YZaf),(Xak,Ca,YZag),(Xak,Ca,YZah),(Xak, Ca,YZai),(Xak,Ca,YZaj),(Xak,Ca,YZak),(Xak,Ca,YZal),(Xak,Ca,YZam),(Xak,Ca,YZan), (Xak,Ca,YZao),(Xak,Ca,YZap),(Xak,Ca,YZaq),(Xak,Ca,YZar),(Xak,Ca,YZas),(Xak, Ca,YZat),(Xak,Ca,YZau),(Xak,Ca,YZav),(Xak,Ca,YZaw),(Xak,Ca,YZax),(Xak,Ca, YZay),(Xak,Ca,YZaz),(Xak,Ca,YZba),(Xak,Ca,YZbb),(Xak,Ca,YZbc),(Xak,Ca,YZbd), (Xak,Ca,YZbe),(Xak,Ca,YZbf),(Xak,Ca,YZbg),(Xak,Ca,YZbh),(Xak,Ca,YZbi),(Xak, Ca,YZbj),(Xak,Ca,YZbk),(Xak,Ca,YZbl),(Xak,Ca,YZbm),(Xak,Ca,YZbn),(Xak,Ca,YZbo), (Xak,Ca,YZbp),(Xak,Ca,YZbq),(Xak,Ca,YZbr),(Xak,Ca,YZbs),(Xak,Ca,YZbt),(Xak, Ca,YZbu),(Xak,Ca,YZbv),(Xak,Ca,YZbw),(Xak,Ca,YZbx),(Xak,Ca,YZby),(Xak, Ca,YZbz),(Xak,Ca,YZca),(Xak,Ca,YZcb),(Xak,Ca,YZcc),(Xak,Ca,YZcd),(Xak,Ca,YZce), (Xak,Ca,YZcf),(Xak,Ca,YZcg),(Xak,Ca,YZch),(Xak,Ca,YZci),(Xak,Ca,YZcj),(Xak, Ca,YZck),(Xak,Ca,YZcl),(Xak,Ca,YZcm),(Xak,Ca,YZcn),(Xak,Ca,YZco),(Xak,Ca, YZcp),(Xak,Ca,YZcq),(Xak,Ca,YZcr),(Xak,Ca,YZcs),(Xak,Ca,YZct),(Xak,Ca,YZcu), (Xak,Ca,YZcv),(Xak,Ca,YZcw),(Xak,Ca,YZcx),(Xak,Ca,YZcy),(Xak,Ca,YZcz),(Xak, Ca,YZda),(Xak,Ca,YZdb),(Xak,Ca,YZdc),(Xak,Ca,YZdd),(Xak,Ca,YZde),(Xak,Ca,YZdf), (Xak,Ca,YZdg),(Xak,Ca,YZdh),(Xak,Ca,YZdi),(Xak,Ca,YZdj),(Xak,Ca,YZdk),(Xak, Ca,YZdl),(Xak,Ca,YZdm),(Xak,Ca,YZdn),(Xak,Ca,YZdo),(Xak,Ca,YZdp),(Xak, Ca,YZdq),(Xak,Ca,YZdr),(Xak,Ca,YZds),(Xak,Ca,Yzdt),(Xak,Ca,YZdu),(Xak,Ca,YZdv), (Xak,Ca,YZdw),(Xak,Ca,YZdx),(Xak,Ca,YZdy),(Xak,Ca,YZdz),(Xak,Ca,YZea),(Xak, Ca,YZeb),(Xak,Ca,YZec),(Xak,Ca,YZed),(Xak,Ca,YZee),(Xak,Ca,YZef),(Xak,Ca, YZeg),(Xak,Ca,YZeh),(Xak,Ca,YZei),(Xak,Ca,YZej),(Xak,Ca,YZek),(Xak,Ca,YZel), (Xak,Ca,YZem),(Xak,Ca,YZen),(Xak,Ca,YZeo),(Xak,Ca,YZep),(Xak,Ca,YZeq),(Xak, Ca,YZer),(Xak,Ca,YZes),(Xak,Ca,YZet),(Xak,Ca,YZeu),(Xak,Ca,YZev),(Xak,Ca,YZew), (Xak,Ca,YZex),(Xak,Ca,YZey),(Xak,Ca,YZez),(Xak,Ca,YZfa),(Xak,Ca,YZfb),(Xak, Ca,YZfc),(Xak,Ca,YZfd),(Xak,Ca,YZfe),(Xak,Ca,YZff),(Xak,Ca,YZfg),(Xak,Ca,YZfh), (Xak,Ca,YZfi),(Xak,Ca,YZfj),(Xak,Ca,YZfk),(Xak,Ca,YZfl),(Xak,Ca,YZfm),(Xak, Ca,YZfn),(Xak,Ca,YZfo),(Xak,Ca,YZfp),(Xak,Ca,YZfq),(Xak,Ca,YZfr),(Xak,Ca,YZfs), (Xak,Ca,YZft),(Xak,Ca,YZfu),(Xak,Ca,YZfv),(Xak,Ca,YZfw),(Xak,Ca,YZfx),(Xak, Ca,YZfy),(Xak,Ca,YZfz),(Xak,Ca,YZga),(Xak,Ca,YZgb),(Xak,Ca,YZgc),(Xak,Ca, YZgd),(Xak,Ca,YZge),(Xak,Ca,YZgf),(Xak,Ca,YZgg),(Xak,Ca,YZgh),(Xak,Ca,YZgi), (Xak,Ca,YZgj),(Xak,Ca,YZgk),(Xak,Ca,YZgl),(Xak,Ca,YZgm),(Xak,Ca,YZgn),(Xak, Ca,YZgo),(Xak,Ca,YZgp),(Xak,Ca,YZgq),(Xak,Ca,YZgr),(Xak,Ca,YZgs),(Xak,Ca,YZgt), (Xak,Cb,YZa),(Xak,Cb,YZb),(Xak,Cb,YZc),(Xak,Cb,YZd),(Xak,Cb,YZe),(Xak, Cb,YZf),(Xak,Cb,YZg),(Xak,Cb,YZh),(Xak,Cb,YZi),(Xak,Cb,YZj),(Xak,Cb,YZk),(Xak, Cb,YZl),(Xak,Cb,YZm),(Xak,Cb,YZn),(Xak,Cb,YZo),(Xak,Cb,YZp),(Xak,Cb,YZq), (Xak,Cb,YZr),(Xak,Cb,YZs),(Xak,Cb,YZt),(Xak,Cb,YZu),(Xak,Cb,YZv),(Xak,Cb,YZw), (Xak,Cb,YZx),(Xak,Cb,YZy),(Xak,Cb,YZz),(Xak,Cb,YZaa),(Xak,Cb,YZab),(Xak, Cb,YZac),(Xak,Cb,YZad),(Xak,Cb,YZae),(Xak,Cb,YZaf),(Xak,Cb,YZag),(Xak,Cb, YZah),(Xak,Cb,YZai),(Xak,Cb,YZaj),(Xak,Cb,YZak),(Xak,Cb,YZal),(Xak,Cb,YZam), (Xak,Cb,YZan),(Xak,Cb,YZao),(Xak,Cb,YZap),(Xak,Cb,YZaq),(Xak,Cb,YZar),(Xak, Cb,YZas),(Xak,Cb,YZat),(Xak,Cb,YZau),(Xak,Cb,YZav),(Xak,Cb,YZaw),(Xak,Cb,YZax), (Xak,Cb,YZay),(Xak,Cb,YZaz),(Xak,Cb,YZba),(Xak,Cb,YZbb),(Xak,Cb,YZbc), (Xak,Cb,YZbd),(Xak,Cb,YZbe),(Xak,Cb,YZbf),(Xak,Cb,YZbg),(Xak,Cb,YZbh),(Xak, Cb,YZbi),(Xak,Cb,YZbj),(Xak,Cb,YZbk),(Xak,Cb,YZbl),(Xak,Cb,YZbm),(Xak,Cb,YZbn), (Xak,Cb,YZbo),(Xak,Cb,YZbp),(Xak,Cb,YZbq),(Xak,Cb,YZbr),(Xak,Cb,YZbs), (Xak,Cb,YZbt),(Xak,Cb,YZbu),(Xak,Cb,YZbv),(Xak,Cb,YZbw),(Xak,Cb,YZbx),(Xak, Cb,YZby),(Xak,Cb,YZbz),(Xak,Cb,YZca),(Xak,Cb,YZcb),(Xak,Cb,YZcc),(Xak,Cb,YZcd), (Xak,Cb,YZce),(Xak,Cb,YZcf),(Xak,Cb,YZcg),(Xak,Cb,YZch),(Xak,Cb,YZci),(Xak, Cb,YZcj),(Xak,Cb,YZck),(Xak,Cb,YZcl),(Xak,Cb,YZcm),(Xak,Cb,YZcn),(Xak, Cb,YZco),(Xak,Cb,YZcp),(Xak,Cb,YZcq),(Xak,Cb,YZcr),(Xak,Cb,YZcs),(Xak,Cb,YZct), (Xak,Cb,YZcu),(Xak,Cb,YZcv),(Xak,Cb,YZcw),(Xak,Cb,YZcx),(Xak,Cb,YZcy), (Xak,Cb,YZcz),(Xak,Cb,YZda),(Xak,Cb,YZdb),(Xak,Cb,YZdc),(Xak,Cb,YZdd),(Xak, Cb,YZde),(Xak,Cb,YZdf),(Xak,Cb,YZdg),(Xak,Cb,YZdh),(Xak,Cb,YZdi),(Xak,Cb,YZdj), (Xak,Cb,YZdk),(Xak,Cb,YZdl),(Xak,Cb,YZdm),(Xak,Cb,YZdn),(Xak,Cb,YZdo), (Xak,Cb,YZdp),(Xak,Cb,YZdq),(Xak,Cb,YZdr),(Xak,Cb,YZds),(Xak,Cb,Yzdt),(Xak, Cb,YZdu),(Xak,Cb,YZdv),(Xak,Cb,YZdw),(Xak,Cb,YZdx),(Xak,Cb,YZdy),(Xak,Cb, YZdz),(Xak,Cb,YZea),(Xak,Cb,YZeb),(Xak,Cb,YZec),(Xak,Cb,YZed),(Xak,Cb,YZee), (Xak,Cb,YZef),(Xak,Cb,YZeg),(Xak,Cb,YZeh),(Xak,Cb,YZei),(Xak,Cb,YZej),(Xak, Cb,YZek),(Xak,Cb,YZel),(Xak,Cb,YZem),(Xak,Cb,YZen),(Xak,Cb,YZeo),(Xak,Cb,YZep), (Xak,Cb,YZeq),(Xak,Cb,YZer),(Xak,Cb,YZes),(Xak,Cb,YZet),(Xak,Cb,YZeu),(Xak, Cb,YZev),(Xak,Cb,YZew),(Xak,Cb,YZex),(Xak,Cb,YZey),(,Xak,Cb,YZez),(Xak, Cb,YZfa),(Xak,Cb,YZfb),(Xak,Cb,YZfc),(Xak,Cb,YZfd),(Xak,Cb,YZfe),(Xak,Cb,YZff), (Xak,Cb,YZfg),(Xak,Cb,YZfh),(Xak,Cb,YZfi),(Xak,Cb,YZfj),(Xak,Cb,YZfk),(Xak, Cb,YZfl),(Xak,Cb,YZfm),(Xak,Cb,YZfn),(Xak,Cb,YZfo),(Xak,Cb,YZfp),(Xak,Cb,YZfq), (Xak,Cb,YZfr),(Xak,Cb,YZfs),(Xak,Cb,YZft),(Xak,Cb,YZfu),(Xak,Cb,YZfv),(Xak, Cb,YZfw),(Xak,Cb,YZfx),(Xak,Cb,YZfy),(Xak,Cb,YZfz),(Xak,Cb,YZga),(Xak,Cb,YZgb), (Xak,Cb,YZgc),(Xak,Cb,YZgd),(Xak,Cb,YZge),(Xak,Cb,YZgf),(Xak,Cb,YZgg), (Xak,Cb,YZgh),(Xak,Cb,YZgi),(Xak,Cb,YZgj),(Xak,Cb,YZgk),(Xak,Cb,YZgl),(Xak, Cb,YZgm),(Xak,Cb,YZgn),(Xak,Cb,YZgo),(Xak,Cb,YZgp),(Xak,Cb,YZgq),(Xak,Cb, YZgr),(Xak,Cb,YZgs),(Xak,Cb,YZgt),(Xak,Cc,YZa),(Xak,Cc,YZb),(Xak,Cc,YZc),(Xak, Cc,YZd),(Xak,Cc,YZe),(Xak,Cc,YZf),(Xak,Cc,YZg),(Xak,Cc,YZh),(Xak,Cc,YZi),(Xak, Cc,YZj),(Xak,Cc,YZk),(Xak,Cc,YZl),(Xak,Cc,YZm),(Xak,Cc,YZn),(Xak,Cc,YZo), (Xak,Cc,YZp),(Xak,Cc,YZq),(Xak,Cc,YZr),(Xak,Cc,YZs),(Xak,Cc,YZt),(Xak,Cc, YZu),(Xak,Cc,YZv),(Xak,Cc,YZw),(Xak,Cc,YZx),(Xak,Cc,YZy),(Xak,Cc,YZz),(Xak, Cc,YZaa),(Xak,Cc,YZab),(Xak,Cc,YZac),(Xak,Cc,YZad),(Xak,Cc,YZae),(Xak,Cc,YZaf), (Xak,Cc,YZag),(Xak,Cc,YZah),(Xak,Cc,YZai),(Xak,Cc,YZaj),(Xak,Cc,YZak),(Xak, Cc,YZal),(Xak,Cc,YZam),(Xak,Cc,YZan),(Xak,Cc,YZao),(Xak,Cc,YZap),(Xak,Cc, YZaq),(Xak,Cc,YZar),(Xak,Cc,YZas),(Xak,Cc,YZat),(Xak,Cc,YZau),(Xak,Cc,YZav), (Xak,Cc,YZaw),(Xak,Cc,YZax),(Xak,Cc,YZay),(Xak,Cc,YZaz),(Xak,Cc,YZba),(Xak, Cc,YZbb),(Xak,Cc,YZbc),(Xak,Cc,YZbd),(Xak,Cc,YZbe),(Xak,Cc,YZbf),(Xak,Cc,YZbg), (Xak,Cc,YZbh),(Xak,Cc,YZbi),(Xak,Cc,YZbj),(Xak,Cc,YZbk),(Xak,Cc,YZbl),(Xak, Cc,YZbm),(Xak,Cc,YZbn),(Xak,Cc,YZbo),(Xak,Cc,YZbp),(Xak,Cc,YZbq),(Xak, Cc,YZbr),(Xak,Cc,YZbs),(Xak,Cc,YZbt),(Xak,Cc,YZbu),(Xak,Cc,YZbv),(Xak,Cc,YZbw), (Xak,Cc,YZbx),(Xak,Cc,YZby),(Xak,Cc,YZbz),(Xak,Cc,YZca),(Xak,Cc,YZcb),(Xak, Cc,YZcc),(Xak,Cc,YZcd),(Xak,Cc,YZce),(Xak,Cc,YZcf),(Xak,Cc,YZcg),(Xak,Cc, YZch),(Xak,Cc,YZci),(Xak,Cc,YZcj),(Xak,Cc,YZck),(Xak,Cc,YZcl),(Xak,Cc,YZcm), (Xak,Cc,YZcn),(Xak,Cc,YZco),(Xak,Cc,YZcp),(Xak,Cc,YZcq),(Xak,Cc,YZcr),(Xak, Cc,YZcs),(Xak,Cc,YZct),(Xak,Cc,YZcu),(Xak,Cc,YZcv),(Xak,Cc,YZcw),(Xak,Cc,YZcx), (Xak,Cc,YZcy),(Xak,Cc,YZcz),(Xak,Cc,YZda),(Xak,Cc,YZdb),(Xak,Cc,YZdc),(Xak, Cc,YZdd),(Xak,Cc,YZde),(Xak,Cc,YZdf),(Xak,Cc,YZdg),(Xak,Cc,YZdh),(Xak,Cc, YZdi),(Xak,Cc,YZdj),(Xak,Cc,YZdk),(Xak,Cc,YZdl),(Xak,Cc,YZdm),(Xak,Cc,YZdn), (Xak,Cc,YZdo),(Xak,Cc,YZdp),(Xak,Cc,YZdq),(Xak,Cc,YZdr),(Xak,Cc,YZds),(Xak, Cc,Yzdt),(Xak,Cc,YZdu),(Xak,Cc,YZdv),(Xak,Cc,YZdw),(Xak,Cc,YZdx),(Xak,Cc, YZdy),(Xak,Cc,YZdz),(Xak,Cc,YZea),(Xak,Cc,YZeb),(Xak,Cc,YZec),(Xak,Cc,YZed), (Xak,Cc,YZee),(Xak,Cc,YZef),(Xak,Cc,YZeg),(Xak,Cc,YZeh),(Xak,Cc,YZei),(Xak, Cc,YZej),(Xak,Cc,YZek),(Xak,Cc,YZel),(Xak,Cc,YZem),(Xak,Cc,YZen),(Xak,Cc,YZeo), (Xak,Cc,YZep),(Xak,Cc,YZeq),(Xak,Cc,YZer),(Xak,Cc,YZes),(Xak,Cc,YZet),(Xak, Cc,YZeu),(Xak,Cc,YZev),(Xak,Cc,YZew),(Xak,Cc,YZex),(Xak,Cc,YZey),(Xak,Cc, YZez),(Xak,Cc,YZfa),(Xak,Cc,YZfb),(Xak,Cc,YZfc),(Xak,Cc,YZfd),(Xak,Cc,YZfe),(Xak, Cc,YZff),(Xak,Cc,YZfg),(Xak,Cc,YZfh),(Xak,Cc,YZfi),(Xak,Cc,YZfj),(Xak,Cc, YZfk),(Xak,Cc,YZfl),(Xak,Cc,YZfm),(Xak,Cc,YZfn),(Xak,Cc,YZfo),(Xak,Cc,YZfp),(Xak, Cc,YZfq),(Xak,Cc,YZfr),(Xak,Cc,YZfs),(Xak,Cc,YZft),(Xak,Cc,YZfu),(Xak,Cc, YZfv),(Xak,Cc,YZfw),(Xak,Cc,YZfx),(Xak,Cc,YZfy),(Xak,Cc,YZfz),(Xak,Cc,YZga), (Xak,Cc,YZgb),(Xak,Cc,YZgc),(Xak,Cc,YZgd),(Xak,Cc,YZge),(Xak,Cc,YZgf),(Xak, Cc,YZgg),(Xak,Cc,YZgh),(Xak,Cc,YZgi),(Xak,Cc,YZgj),(Xak,Cc,YZgk),(Xak,Cc,YZgl), (Xak,Cc,YZgm),(Xak,Cc,YZgn),(Xak,Cc,YZgo),(Xak,Cc,YZgp),(Xak,Cc,YZgq),(Xak, Cc,YZgr),(Xak,Cc,YZgs),(Xak,Cc,YZgt),(Xak,Cd,YZa),(Xak,Cd,YZb),(Xak,Cd, YZc),(Xak,Cd,YZd),(Xak,Cd,YZe),(Xak,Cd,YZf),(Xak,Cd,YZg),(Xak,Cd,YZh),(Xak, Cd,YZi),(Xak,Cd,YZj),(Xak,Cd,YZk),(Xak,Cd,YZl),(Xak,Cd,YZm),(Xak,Cd,YZn),(Xak, Cd,YZo),(Xak,Cd,YZp),(Xak,Cd,YZq),(Xak,Cd,YZr),(Xak,Cd,YZs),(Xak,Cd,YZt), (Xak,Cd,YZu),(Xak,Cd,YZv),(Xak,Cd,YZw),(Xak,Cd,YZx),(Xak,Cd,YZy),(Xak,Cd, YZz),(Xak,Cd,YZaa),(Xak,Cd,YZab),(Xak,Cd,YZac),(Xak,Cd,YZad),(Xak,Cd,YZae), (Xak,Cd,YZaf),(Xak,Cd,YZag),(Xak,Cd,YZah),(Xak,Cd,YZai),(Xak,Cd,YZaj),(Xak,Cd, YZak),(Xak,Cd,YZal),(Xak,Cd,YZam),(Xak,Cd,YZan),(Xak,Cd,YZao),(Xak,Cd,YZap), (Xak,Cd,YZaq),(Xak,Cd,YZar),(Xak,Cd,YZas),(Xak,Cd,YZat),(Xak,Cd,YZau),(Xak, Cd,YZav),(Xak,Cd,YZaw),(Xak,Cd,YZax),(Xak,Cd,YZay),(Xak,Cd,YZaz),(Xak,Cd, YZba),(Xak,Cd,YZbb),(Xak,Cd,YZbc),(Xak,Cd,YZbd),(Xak,Cd,YZbe),(Xak,Cd,YZbf), (Xak,Cd,YZbg),(Xak,Cd,YZbh),(Xak,Cd,YZbi),(Xak,Cd,YZbj),(Xak,Cd,YZbk),(Xak, Cd,YZbl),(Xak,Cd,YZbm),(Xak,Cd,YZbn),(Xak,Cd,YZbo),(Xak,Cd,YZbp),(Xak,Cd, YZbq),(Xak,Cd,YZbr),(Xak,Cd,YZbs),(Xak,Cd,YZbt),(Xak,Cd,YZbu),(Xak,Cd,YZbv), (Xak,Cd,YZbw),(Xak,Cd,YZbx),(Xak,Cd,YZby),(Xak,Cd,YZbz),(Xak,Cd,YZca),(Xak, Cd,YZcb),(Xak,Cd,YZcc),(Xak,Cd,YZcd),(Xak,Cd,YZce),(Xak,Cd,YZcf),(Xak,Cd, YZcg),(Xak,Cd,YZch),(Xak,Cd,YZci),(Xak,Cd,YZcj),(Xak,Cd,YZck),(Xak,Cd,YZcl), (Xak,Cd,YZcm),(Xak,Cd,YZcn),(Xak,Cd,YZco),(Xak,Cd,YZcp),(Xak,Cd,YZcq),(Xak, Cd,YZcr),(Xak,Cd,YZcs),(Xak,Cd,YZct),(Xak,Cd,YZcu),(Xak,Cd,YZcv),(Xak,Cd, YZcw),(Xak,Cd,YZcx),(Xak,Cd,YZcy),(Xak,Cd,YZcz),(Xak,Cd,YZda),(Xak,Cd,YZdb), (Xak,Cd,YZdc),(Xak,Cd,YZdd),(Xak,Cd,YZde),(Xak,Cd,YZdf),(Xak,Cd,YZdg),(Xak, Cd,YZdh),(Xak,Cd,YZdi),(Xak,Cd,YZdj),(Xak,Cd,YZdk),(Xak,Cd,YZdl),(Xak,Cd, YZdm),(Xak,Cd,YZdn),(Xak,Cd,YZdo),(Xak,Cd,YZdp),(Xak,Cd,YZdq),(Xak,Cd,YZdr), (Xak,Cd,YZds),(Xak,Cd,Yzdt),(Xak,Cd,YZdu),(Xak,Cd,YZdv),(Xak,Cd,YZdw),(Xak, Cd,YZdx),(Xak,Cd,YZdy),(Xak,Cd,YZdz),(Xak,Cd,YZea),(Xak,Cd,YZeb),(Xak, Cd,YZec),(Xak,Cd,YZed),(Xak,Cd,YZee),(Xak,Cd,YZef),(Xak,Cd,YZeg),(Xak,Cd,YZeh), (Xak,Cd,YZei),(Xak,Cd,YZej),(Xak,Cd,YZek),(Xak,Cd,YZel),(Xak,Cd,YZem),(Xak, Cd,YZen),(Xak,Cd,YZeo),(Xak,Cd,YZep),(Xak,Cd,YZeq),(Xak,Cd,YZer),(Xak, Cd,YZes),(Xak,Cd,YZet),(Xak,Cd,YZeu),(Xak,Cd,YZev),(Xak,Cd,YZew),(Xak,Cd,YZex), (Xak,Cd,YZey),(Xak,Cd,YZez),(Xak,Cd,YZfa),(Xak,Cd,YZfb),(Xak,Cd,YZfc),(Xak, Cd,YZfd),(Xak,Cd,YZfe),(Xak,Cd,YZff),(Xak,Cd,YZfg),(Xak,Cd,YZfh),(Xak,Cd, YZfi),(Xak,Cd,YZfj),(Xak,Cd,YZfk),(Xak,Cd,YZfl),(Xak,Cd,YZfm),(Xak,Cd,YZfn), (Xak,Cd,YZfo),(Xak,Cd,YZfp),(Xak,Cd,YZfq),(Xak,Cd,YZfr),(Xak,Cd,YZfs),(Xak,Cd, YZft),(Xak,Cd,YZfu),(Xak,Cd,YZfv),(Xak,Cd,YZfw),(Xak,Cd,YZfx),(Xak,Cd,YZfy), (Xak,Cd,YZfz),(Xak,Cd,YZga),(Xak,Cd,YZgb),(Xak,Cd,YZgc),(Xak,Cd,YZgd),(Xak, Cd,YZge),(Xak,Cd,YZgf),(Xak,Cd,YZgg),(Xak,Cd,YZgh),(Xak,Cd,YZgi),(Xak,Cd, YZgj),(Xak,Cd,YZgk),(Xak,Cd,YZgl),(Xak,Cd,YZgm),(Xak,Cd,YZgn),(Xak,Cd,YZgo), (Xak,Cd,YZgp),(Xak,Cd,YZgq),(Xak,Cd,YZgr),(Xak,Cd,YZgs),(Xak,Cd,YZgt),(Xak, Ce,YZa),(Xak,Ce,YZb),(Xak,Ce,YZc),(Xak,Ce,YZd),(Xak,Ce,YZe),(Xak,Ce,YZf),(Xak, Ce,YZg),(Xak,Ce,YZh),(Xak,Ce,YZi),(Xak,Ce,YZj),(Xak,Ce,YZk),(Xak,Ce,YZl), (Xak,Ce,YZm),(Xak,Ce,YZn),(Xak,Ce,YZo),(Xak,Ce,YZp),(Xak,Ce,YZq),(Xak,Ce,YZr), (Xak,Ce,YZs),(Xak,Ce,YZt),(Xak,Ce,YZu),(Xak,Ce,YZv),(Xak,Ce,YZw),(Xak,Ce, YZx),(Xak,Ce,YZy),(Xak,Ce,YZz),(Xak,Ce,YZaa),(Xak,Ce,YZab),(Xak,Ce,YZac),(Xak, Ce,YZad),(Xak,Ce,YZae),(Xak,Ce,YZaf),(Xak,Ce,YZag),(Xak,Ce,YZah),(Xak,Ce, YZai),(Xak,Ce,YZaj),(Xak,Ce,YZak),(Xak,Ce,YZal),(Xak,Ce,YZam),(Xak,Ce,YZan), (Xak,Ce,YZao),(Xak,Ce,YZap),(Xak,Ce,YZaq),(Xak,Ce,YZar),(Xak,Ce,YZas),(Xak, Ce,YZat),(Xak,Ce,YZau),(Xak,Ce,YZav),(Xak,Ce,YZaw),(Xak,Ce,YZax),(Xak,Ce,YZay), (Xak,Ce,YZaz),(Xak,Ce,YZba),(Xak,Ce,YZbb),(Xak,Ce,YZbc),(Xak,Ce,YZbd),(Xak, Ce,YZbe),(Xak,Ce,YZbf),(Xak,Ce,YZbg),(Xak,Ce,YZbh),(Xak,Ce,YZbi),(Xak,Ce, YZbj),(Xak,Ce,YZbk),(Xak,Ce,YZbl),(Xak,Ce,YZbm),(Xak,Ce,YZbn),(Xak,Ce,YZbo), (Xak,Ce,YZbp),(Xak,Ce,YZbq),(Xak,Ce,YZbr),(Xak,Ce,YZbs),(Xak,Ce,YZbt),(Xak, Ce,YZbu),(Xak,Ce,YZbv),(Xak,Ce,YZbw),(Xak,Ce,YZbx),(Xak,Ce,YZby),(Xak,Ce, YZbz),(Xak,Ce,YZca),(Xak,Ce,YZcb),(Xak,Ce,YZcc),(Xak,Ce,YZcd),(Xak,Ce,YZce), (Xak,Ce,YZcf),(Xak,Ce,YZcg),(Xak,Ce,YZch),(Xak,Ce,YZci),(Xak,Ce,YZcj),(Xak,Ce, YZck),(Xak,Ce,YZcl),(Xak,Ce,YZcm),(Xak,Ce,YZcn),(Xak,Ce,YZco),(Xak,Ce,YZcp), (Xak,Ce,YZcq),(Xak,Ce,YZcr),(Xak,Ce,YZcs),(Xak,Ce,YZct),(Xak,Ce,YZcu),(Xak, Ce,YZcv),(Xak,Ce,YZcw),(Xak,Ce,YZcx),(Xak,Ce,YZcy),(Xak,Ce,YZcz),(Xak,Ce,YZda), (Xak,Ce,YZdb),(Xak,Ce,YZdc),(Xak,Ce,YZdd),(Xak,Ce,YZde),(Xak,Ce,YZdf),(Xak, Ce,YZdg),(Xak,Ce,YZdh),(Xak,Ce,YZdi),(Xak,Ce,YZdj),(Xak,Ce,YZdk),(Xak,Ce, YZdl),(Xak,Ce,YZdm),(Xak,Ce,YZdn),(Xak,Ce,YZdo),(Xak,Ce,YZdp),(Xak,Ce,YZdq), (Xak,Ce,YZdr),(Xak,Ce,YZds),(Xak,Ce,Yzdt),(Xak,Ce,YZdu),(Xak,Ce,YZdv),(Xak, Ce,YZdw),(Xak,Ce,YZdx),(Xak,Ce,YZdy),(Xak,Ce,YZdz),(Xak,Ce,YZea),(Xak,Ce, YZeb),(Xak,Ce,YZec),(Xak,Ce,YZed),(Xak,Ce,YZee),(Xak,Ce,YZef),(Xak,Ce,YZeg), (Xak,Ce,YZeh),(Xak,Ce,YZei),(Xak,Ce,YZej),(Xak,Ce,YZek),(Xak,Ce,YZel),(Xak,Ce, YZem),(Xak,Ce,YZen),(Xak,Ce,YZeo),(Xak,Ce,YZep),(Xak,Ce,YZeq),(Xak,Ce,YZer), (Xak,Ce,YZes),(Xak,Ce,YZet),(Xak,Ce,YZeu),(Xak,Ce,YZev),(Xak,Ce,YZew),(Xak, Ce,YZex),(Xak,Ce,YZey),(Xak,Ce,YZez),(Xak,Ce,YZfa),(Xak,Ce,YZfb),(Xak,Ce, YZfc),(Xak,Ce,YZfd),(Xak,Ce,YZfe),(Xak,Ce,YZff),(Xak,Ce,YZfg),(Xak,Ce,YZfh),(Xak, Ce,YZfi),(Xak,Ce,YZfj),(Xak,Ce,YZfk),(Xak,Ce,YZfl),(Xak,Ce,YZfm),(Xak,Ce, YZfn),(Xak,Ce,YZfo),(Xak,Ce,YZfp),(Xak,Ce,YZfq),(Xak,Ce,YZfr),(Xak,Ce,YZfs),(Xak, Ce,YZft),(Xak,Ce,YZfu),(Xak,Ce,YZfv),(Xak,Ce,YZfw),(Xak,Ce,YZfx),(Xak,Ce, YZfy),(Xak,Ce,YZfz),(Xak,Ce,YZga),(Xak,Ce,YZgb),(Xak,Ce,YZgc),(Xak,Ce,YZgd), (Xak,Ce,YZge),(Xak,Ce,YZgf),(Xak,Ce,YZgg),(Xak,Ce,YZgh),(Xak,Ce,YZgi),(Xak, Ce,YZgj),(Xak,Ce,YZgk),(Xak,Ce,YZgl),(Xak,Ce,YZgm),(Xak,Ce,YZgn),(Xak,Ce,YZgo), (Xak,Ce,YZgp),(Xak,Ce,YZgq),(Xak,Ce,YZgr),(Xak,Ce,YZgs),(Xak,Ce,YZgt),(Xak, Cf,YZa),(Xak,Cf,YZb),(Xak,Cf,YZc),(Xak,Cf,YZd),(Xak,Cf,YZe),(Xak,Cf,YZf), (Xak,Cf,YZg),(Xak,Cf,YZh),(Xak,Cf,YZi),(Xak,Cf,YZj),(Xak,Cf,YZk),(Xak,Cf,YZl), (Xak,Cf,YZm),(Xak,Cf,YZn),(Xak,Cf,YZo),(Xak,Cf,YZp),(Xak,Cf,YZq),(Xak,Cf,YZr), (Xak,Cf,YZs),(Xak,Cf,YZt),(Xak,Cf,YZu),(Xak,Cf,YZv),(Xak,Cf,YZw),(Xak,Cf,YZx), (Xak,Cf,YZy),(Xak,Cf,YZz),(Xak,Cf,YZaa),(Xak,Cf,YZab),(Xak,Cf,YZac),(Xak, Cf,YZad),(Xak,Cf,YZae),(Xak,Cf,YZaf),(Xak,Cf,YZag),(Xak,Cf,YZah),(Xak,Cf,YZai), (Xak,Cf,YZaj),(Xak,Cf,YZak),(Xak,Cf,YZal),(Xak,Cf,YZam),(Xak,Cf,YZan),(Xak, Cf,YZao),(Xak,Cf,YZap),(Xak,Cf,YZaq),(Xak,Cf,YZar),(Xak,Cf,YZas),(Xak,Cf,YZat), (Xak,Cf,YZau),(Xak,Cf,YZav),(Xak,Cf,YZaw),(Xak,Cf,YZax),(Xak,Cf,YZay),(Xak, Cf,YZaz),(Xak,Cf,YZba),(Xak,Cf,YZbb),(Xak,Cf,YZbc),(Xak,Cf,YZbd),(Xak,Cf,YZbe), (Xak,Cf,YZbf),(Xak,Cf,YZbg),(Xak,Cf,YZbh),(Xak,Cf,YZbi),(Xak,Cf,YZbj),(Xak, Cf,YZbk),(Xak,Cf,YZbl),(Xak,Cf,YZbm),(Xak,Cf,YZbn),(Xak,Cf,YZbo),(Xak,Cf,YZbp), (Xak,Cf,YZbq),(Xak,Cf,YZbr),(Xak,Cf,YZbs),(Xak,Cf,YZbt),(Xak,Cf,YZbu),(Xak, Cf,YZbv),(Xak,Cf,YZbw),(Xak,Cf,YZbx),(Xak,Cf,YZby),(Xak,Cf,YZbz),(Xak,Cf,YZca), (Xak,Cf,YZcb),(Xak,Cf,YZcc),(Xak,Cf,YZcd),(Xak,Cf,YZce),(Xak,Cf,YZcf),(Xak, Cf,YZcg),(Xak,Cf,YZch),(Xak,Cf,YZci),(Xak,Cf,YZcj),(Xak,Cf,YZck),(Xak,Cf,YZcl), (Xak,Cf,YZcm),(Xak,Cf,YZcn),(Xak,Cf,YZco),(Xak,Cf,YZcp),(Xak,Cf,YZcq),(Xak, Cf,YZcr),(Xak,Cf,YZcs),(Xak,Cf,YZct),(Xak,Cf,YZcu),(Xak,Cf,YZcv),(Xak,Cf, YZcw),(Xak,Cf,YZcx),(Xak,Cf,YZcy),(Xak,Cf,YZcz),(Xak,Cf,YZda),(Xak,Cf,YZdb), (Xak,Cf,YZdc),(Xak,Cf,YZdd),(Xak,Cf,YZde),(Xak,Cf,YZdf),(Xak,Cf,YZdg),(Xak,Cf, YZdh),(Xak,Cf,YZdi),(Xak,Cf,YZdj),(Xak,Cf,YZdk),(Xak,Cf,YZdl),(Xak,Cf,YZdm), (Xak,Cf,YZdn),(Xak,Cf,YZdo),(Xak,Cf,YZdp),(Xak,Cf,YZdq),(Xak,Cf,YZdr),(Xak, Cf,YZds),(Xak,Cf,Yzdt),(Xak,Cf,YZdu),(Xak,Cf,YZdv),(Xak,Cf,YZdw),(Xak,Cf,YZdx), (Xak,Cf,YZdy),(Xak,Cf,YZdz),(Xak,Cf,YZea),(Xak,Cf,YZeb),(Xak,Cf,YZec),(Xak, Cf,YZed),(Xak,Cf,YZee),(Xak,Cf,YZef),(Xak,Cf,YZeg),(Xak,Cf,YZeh),(Xak,Cf,YZei), (Xak,Cf,YZej),(Xak,Cf,YZek),(Xak,Cf,YZel),(Xak,Cf,YZem),(Xak,Cf,YZen),(Xak, Cf,YZeo),(Xak,Cf,YZep),(Xak,Cf,YZeq),(Xak,Cf,YZer),(Xak,Cf,YZes),(Xak,Cf,YZet), (Xak,Cf,YZeu),(Xak,Cf,YZev),(Xak,Cf,YZew),(Xak,Cf,YZex),(Xak,Cf,YZey),(Xak, Cf,YZez),(Xak,Cf,YZfa),(Xak,Cf,YZfb),(Xak,Cf,YZfc),(Xak,Cf,YZfd),(Xak,Cf,YZfe), (Xak,Cf,YZff),(Xak,Cf,YZfg),(Xak,Cf,YZfh),(Xak,Cf,YZfi),(Xak,Cf,YZfj),(Xak,Cf, YZfk),(Xak,Cf,YZfl),(Xak,Cf,YZfm),(Xak,Cf,YZfn),(Xak,Cf,YZfo),(Xak,Cf,YZfp),(Xak, Cf,YZfq),(Xak,Cf,YZfr),(Xak,Cf,YZfs),(Xak,Cf,YZft),(Xak,Cf,YZfu),(Xak,Cf,YZfv), (Xak,Cf,YZfw),(Xak,Cf,YZfx),(Xak,Cf,YZfy),(Xak,Cf,YZfz),(Xak,Cf,YZga),(Xak, Cf,YZgb),(Xak,Cf,YZgc),(Xak,Cf,YZgd),(Xak,Cf,YZge),(Xak,Cf,YZgf),(Xak,Cf,YZgg), (Xak,Cf,YZgh),(Xak,Cf,YZgi),(Xak,Cf,YZgj),(Xak,Cf,YZgk),(Xak,Cf,YZgl),(Xak, Cf,YZgm),(Xak,Cf,YZgn),(Xak,Cf,YZgo),(Xak,Cf,YZgp),(Xak,Cf,YZgq),(Xak,Cf, YZgr),(Xak,Cf,YZgs),(Xak,Cf,YZgt), (Xal,Ca,YZa),(Xal,Ca,YZb),(Xal,Ca,YZc),(Xal,Ca,YZd),(Xal,Ca,YZe),(Xal,Ca,YZf),(Xal, Ca,YZg),(Xal,Ca,YZh),(Xal,Ca,YZi),(Xal,Ca,YZj),(Xal,Ca,YZk),(Xal,Ca,YZl),(Xal, Ca,YZm),(Xal,Ca,YZn),(Xal,Ca,YZo),(Xal,Ca,YZp),(Xal,Ca,YZq),(Xal,Ca,YZr),(Xal, Ca,YZs),(Xal,Ca,YZt),(Xal,Ca,YZu),(Xal,Ca,YZv),(Xal,Ca,YZw),(Xal,Ca,YZx),(Xal, Ca,YZy),(Xal,Ca,YZz),(Xal,Ca,YZaa),(Xal,Ca,YZab),(Xal,Ca,YZac),(Xal,Ca,YZad), (Xal,Ca,YZae),(Xal,Ca,YZaf),(Xal,Ca,YZag),(Xal,Ca,YZah),(Xal,Ca,YZai),(Xal,Ca, YZaj),(Xal,Ca,YZak),(Xal,Ca,YZal),(Xal,Ca,YZam),(Xal,Ca,YZan),(Xal,Ca,YZao),(Xal, Ca,YZap),(Xal,Ca,YZaq),(Xal,Ca,YZar),(Xal,Ca,YZas),(Xal,Ca,YZat),(Xal,Ca,YZau), (Xal,Ca,YZav),(Xal,Ca,YZaw),(Xal,Ca,YZax),(Xal,Ca,YZay),(Xal,Ca,YZaz),(Xal, Ca,YZba),(Xal,Ca,YZbb),(Xal,Ca,YZbc),(Xal,Ca,YZbd),(Xal,Ca,YZbe),(Xal,Ca,YZbf), (Xai,Ca,YZbg),(Xal,Ca,YZbh),(Xal,Ca,YZbi),(Xal,Ca,YZbj),(Xal,Ca,YZbk),(Xal,Ca, YZbl),(Xal,Ca,YZbm),(Xal,Ca,YZbn),(Xal,Ca,YZbo),(Xal,Ca,YZbp),(Xal,Ca,YZbq), (Xal,Ca,YZbr),(Xal,Ca,YZbs),(Xal,Ca,YZbt),(Xal,Ca,YZbu),(Xal,Ca,YZbv),(Xal,Ca, YZbw),(Xal,Ca,YZbx),(Xal,Ca,YZby),(Xal,Ca,YZbz),(Xal,Ca,YZca),(Xal,Ca,YZcb),(Xal, Ca,YZcc),(Xal,Ca,YZcd),(Xal,Ca,YZce),(Xal,Ca,YZcf),(Xal,Ca,YZcg),(Xal,Ca,YZch), (Xal,Ca,YZci),(Xal,Ca,YZcj),(Xal,Ca,YZck),(Xal,Ca,YZcl),(Xal,Ca,YZcm),(Xal, Ca,YZcn),(Xal,Ca,YZco),(Xal,Ca,YZcp),(Xal,Ca,YZcq),(Xal,Ca,YZcr),(Xal,Ca,YZcs), (Xal,Ca,YZct),(Xal,Ca,YZcu),(Xal,Ca,YZcv),(Xal,Ca,YZcw),(Xal,Ca,YZcx),(Xal,Ca, YZcy),(Xal,Ca,YZcz),(Xal,Ca,YZda),(Xal,Ca,YZdb),(Xal,Ca,YZdc),(Xal,Ca,YZdd),(Xal, Ca,YZde),(Xal,Ca,YZdf),(Xal,Ca,YZdg),(Xal,Ca,YZdh),(Xal,Ca,YZdi),(Xal,Ca,YZdj), (Xal,Ca,YZdk),(Xal,Ca,YZdl),(Xal,Ca,YZdm),(Xal,Ca,YZdn),(Xal,Ca,YZdo),(Xal, Ca,YZdp),(Xal,Ca,YZdq),(Xal,Ca,YZdr),(Xal,Ca,YZds),(Xal,Ca,Yzdt),(Xal,Ca,YZdu), (Xal,Ca,YZdv),(Xal,Ca,YZdw),(Xal,Ca,YZdx),(Xal,Ca,YZdy),(Xal,Ca,YZdz),(Xal, Ca,YZea),(Xal,Ca,YZeb),(Xal,Ca,YZec),(Xal,Ca,YZed),(Xal,Ca,YZee),(Xal,Ca,YZef), (Xal,Ca,YZeg),(Xal,Ca,YZeh),(Xal,Ca,YZei),(Xal,Ca,YZej),(Xal,Ca,YZek),(Xal,Ca, YZel),(Xal,Ca,YZem),(Xal,Ca,YZen),(Xal,Ca,YZeo),(Xal,Ca,YZep),(Xal,Ca,YZeq),(Xal, Ca,YZer),(Xal,Ca,YZes),(Xal,Ca,YZet),(Xal,Ca,YZeu),(Xal,Ca,YZev),(Xal,Ca,YZew), (Xal,Ca,YZex),(Xal,Ca,YZey),(Xal,Ca,YZez),(Xal,Ca,YZfa),(Xal,Ca,YZfb),(Xal, Ca,YZfc),(Xal,Ca,YZfd),(Xal,Ca,YZfe),(Xal,Ca,YZff),(Xal,Ca,YZfg),(Xal,Ca,YZfh), (Xal,Ca,YZfi),(Xal,Ca,YZfj),(Xal,Ca,YZfk),(Xal,Ca,YZfl),(Xal,Ca,YZfm),(Xal,Ca,YZfn), (Xal,Ca,YZfo),(Xal,Ca,YZfp),(Xal,Ca,YZfq),(Xal,Ca,YZfr),(Xal,Ca,YZfs),(Xal, Ca,YZft),(Xal,Ca,YZfu),(Xal,Ca,YZfv),(Xal,Ca,YZfw),(Xal,Ca,YZfx),(Xal,Ca,YZfy), (Xal,Ca,YZfz),(Xal,Ca,YZga),(Xal,Ca,YZgb),(Xal,Ca,YZgc),(Xal,Ca,YZgd),(Xal,Ca, YZge),(Xal,Ca,YZgf),(Xal,Ca,YZgg),(Xal,Ca,YZgh),(Xal,Ca,YZgi),(Xal,Ca,YZgj),(Xal, Ca,YZgk),(Xal,Ca,YZgl),(Xal,Ca,YZgm),(Xal,Ca,YZgn),(Xal,Ca,YZgo),(Xal,Ca,YZgp), (Xal,Ca,YZgq),(Xal,Ca,YZgr),(Xal,Ca,YZgs),(Xal,Ca,YZgt),(Xal,Cb,YZa),(Xal, Cb,YZb),(Xal,Cb,YZc),(Xal,Cb,YZd),(Xal,Cb,YZe),(Xal,Cb,YZf),(Xal,Cb,YZg),(Xal, Cb,YZh),(Xal,Cb,YZi),(Xal,Cb,YZj),(Xal,Cb,YZk),(Xal,Cb,YZl),(Xal,Cb,YZm),(Xal, Cb,YZn),(Xal,Cb,YZo),(Xal,Cb,YZp),(Xal,Cb,YZq),(Xal,Cb,YZr),(Xal,Cb,YZs),(Xal, Cb,YZt),(Xal,Cb,YZu),(Xal,Cb,YZv),(Xal,Cb,YZw),(Xal,Cb,YZx),(Xal,Cb,YZy),(Xal, Cb,YZz),(Xal,Cb,YZaa),(Xal,Cb,YZab),(Xal,Cb,YZac),(Xal,Cb,YZad),(Xal,Cb,YZae), (Xal,Cb,YZaf),(Xal,Cb,YZag),(Xal,Cb,YZah),(Xal,Cb,YZai),(Xal,Cb,YZaj),(Xal,Cb, YZak),(Xal,Cb,YZal),(Xal,Cb,YZam),(Xal,Cb,YZan),(Xal,Cb,YZao),(Xal,Cb,YZap),(Xal, Cb,YZaq),(Xal,Cb,YZar),(Xal,Cb,YZas),(Xal,Cb,YZat),(Xal,Cb,YZau),(Xal,Cb,YZav), (Xal,Cb,YZaw),(Xal,Cb,YZax),(Xal,Cb,YZay),(Xal,Cb,YZaz),(Xal,Cb,YZba),(Xal, Cb,YZbb),(Xal,Cb,YZbc),(Xal,Cb,YZbd),(Xal,Cb,YZbe),(Xal,Cb,YZbf),(Xal,Cb,YZbg), (Xal,Cb,YZbh),(Xal,Cb,YZbi),(Xal,Cb,YZbj),(Xal,Cb,YZbk),(Xal,Cb,YZbl),(Xal, Cb,YZbm),(Xal,Cb,YZbn),(Xal,Cb,YZbo),(Xal,Cb,YZbp),(Xal,Cb,YZbq),(Xal,Cb,YZbr), (Xal,Cb,YZbs),(Xal,Cb,YZbt),(Xal,Cb,YZbu),(Xal,Cb,YZbv),(Xal,Cb,YZbw),(Xal, Cb,YZbx),(Xal,Cb,YZby),(Xal,Cb,YZbz),(Xal,Cb,YZca),(Xal,Cb,YZcb),(Xal,Cb,YZcc), (Xal,Cb,YZcd),(Xal,Cb,YZce),(Xal,Cb,YZcf),(Xal,Cb,YZcg),(Xal,Cb,YZch),(Xal, Cb,YZci),(Xal,Cb,YZcj),(Xal,Cb,YZck),(Xal,Cb,YZcl),(Xal,Cb,YZcm),(Xal,Cb,YZcn), (Xal,Cb,YZco),(Xal,Cb,YZcp),(Xal,Cb,YZcq),(Xal,Cb,YZcr),(Xal,Cb,YZcs),(Xal, Cb,YZct),(Xal,Cb,YZcu),(Xal,Cb,YZcv),(Xal,Cb,YZcw),(Xal,Cb,YZcx),(Xal,Cb,YZcy), (Xal,Cb,YZcz),(Xal,Cb,YZda),(Xal,Cb,YZdb),(Xal,Cb,YZdc),(Xal,Cb,YZdd),(Xal, Cb,YZde),(Xal,Cb,YZdf),(Xal,Cb,YZdg),(Xal,Cb,YZdh),(Xal,Cb,YZdi),(Xal,Cb,YZdj), (Xal,Cb,YZdk),(Xal,Cb,YZdl),(Xal,Cb,YZdm),(Xal,Cb,YZdn),(Xal,Cb,YZdo),(Xal, Cb,YZdp),(Xal,Cb,YZdq),(Xal,Cb,YZdr),(Xal,Cb,YZds),(Xal,Cb,Yzdt),(Xal,Cb,YZdu), (Xal,Cb,YZdv),(Xal,Cb,YZdw),(Xal,Cb,YZdx),(Xal,Cb,YZdy),(Xal,Cb,YZdz),(Xal, Cb,YZea),(Xal,Cb,YZeb),(Xal,Cb,YZec),(Xal,Cb,YZed),(Xal,Cb,YZee),(Xal,Cb,YZef), (Xal,Cb,YZeg),(Xal,Cb,YZeh),(Xal,Cb,YZei),(Xal,Cb,YZej),(Xal,Cb,YZek),(Xal,Cb, YZel),(Xal,Cb,YZem),(Xal,Cb,YZen),(Xal,Cb,YZeo),(Xal,Cb,YZep),(Xal,Cb,YZeq), (Xal,Cb,YZer),(Xal,Cb,YZes),(Xal,Cb,YZet),(Xal,Cb,YZeu),(Xal,Cb,YZev),(Xal,Cb, YZew),(Xal,Cb,YZex),(Xal,Cb,YZey),(Xal,Cb,YZez),(Xal,Cb,YZfa),(Xal,Cb,YZfb),(Xal, Cb,YZfc),(Xal,Cb,YZfd),(Xal,Cb,YZfe),(Xal,Cb,YZff),(Xal,Cb,YZfg),(Xal,Cb,YZfh), (Xal,Cb,YZfi),(Xal,Cb,YZfj),(Xal,Cb,YZfk),(Xal,Cb,YZfl),(Xal,Cb,YZfm),(Xal, Cb,YZfn),(Xal,Cb,YZfo),(Xal,Cb,YZfp),(Xal,Cb,YZfq),(Xal,Cb,YZfr),(Xal,Cb,YZfs), (Xal,Cb,YZft),(Xal,Cb,YZfu),(Xal,Cb,YZfv),(Xal,Cb,YZfw),(Xal,Cb,YZfx),(Xal,Cb, YZfy),(Xal,Cb,YZfz),(Xal,Cb,YZga),(Xal,Cb,YZgb),(Xal,Cb,YZgc),(Xal,Cb,YZgd),(Xal, Cb,YZge),(Xal,Cb,YZgf),(Xal,Cb,YZgg),(Xal,Cb,YZgh),(Xal,Cb,YZgi),(Xal,Cb, YZgj),(Xal,Cb,YZgk),(Xal,Cb,YZgl),(Xal,Cb,YZgm),(Xal,Cb,YZgn),(Xal,Cb,YZgo),(Xal, Cb,YZgp),(Xal,Cb,YZgq),(Xal,Cb,YZgr),(Xal,Cb,YZgs),(Xal,Cb,YZgt),(Xal,Cc, YZa),(Xal,Cc,YZb),(Xal,Cc,YZc),(Xal,Cc,YZd),(Xal,Cc,YZe),(Xal,Cc,YZf),(Xal,Cc, YZg),(Xal,Cc,YZh),(Xal,Cc,YZi),(Xal,Cc,YZj),(Xal,Cc,YZk),(Xal,Cc,YZl),(Xal,Cc,YZm), (Xal,Cc,YZn),(Xal,Cc,YZo),(Xal,Cc,YZp),(Xal,Cc,YZq),(Xal,Cc,YZr),(Xal,Cc,YZs), (Xal,Cc,YZt),(Xal,Cc,YZu),(Xal,Cc,YZv),(Xal,Cc,YZw),(Xal,Cc,YZx),(Xal,Cc,YZy), (Xal,Cc,YZz),(Xal,Cc,YZaa),(Xal,Cc,YZab),(Xal,Cc,YZac),(Xal,Cc,YZad),(Xal, Cc,YZae),(Xal,Cc,YZaf),(Xal,Cc,YZag),(Xal,Cc,YZah),(Xal,Cc,YZai),(Xal,Cc,YZaj), (Xal,Cc,YZak),(Xal,Cc,YZal),(Xal,Cc,YZam),(Xal,Cc,YZan),(Xal,Cc,YZao),(Xal,Cc, YZap),(Xal,Cc,YZaq),(Xal,Cc,YZar),(Xal,Cc,YZas),(Xal,Cc,YZat),(Xal,Cc,YZau),(Xal, Cc,YZav),(Xal,Cc,YZaw),(Xal,Cc,YZax),(Xal,Cc,YZay),(Xal,Cc,YZaz),(Xal,Cc,YZba), (Xal,Cc,YZbb),(Xal,Cc,YZbc),(Xal,Cc,YZbd),(Xal,Cc,YZbe),(Xal,Cc,YZbf),(Xal, Cc,YZbg),(Xal,Cc,YZbh),(Xal,Cc,YZbi),(Xal,Cc,YZbj),(Xal,Cc,YZbk),(Xal,Cc,YZbl), (Xal,Cc,YZbm),(Xal,Cc,YZbn),(Xal,Cc,YZbo),(Xal,Cc,YZbp),(Xal,Cc,YZbq),(Xal, Cc,YZbr),(Xal,Cc,YZbs),(Xal,Cc,YZbt),(Xal,Cc,YZbu),(Xal,Cc,YZbv),(Xal,Cc,YZbw), (Xal,Cc,YZbx),(Xal,Cc,YZby),(Xal,Cc,YZbz),(Xal,Cc,YZca),(Xal,Cc,YZcb),(Xal,Cc, YZcc),(Xal,Cc,YZcd),(Xal,Cc,YZce),(Xal,Cc,YZcf),(Xal,Cc,YZcg),(Xal,Cc,YZch),(Xal, Cc,YZci),(Xal,Cc,YZcj),(Xal,Cc,YZck),(Xal,Cc,YZcl),(Xal,Cc,YZcm),(Xal,Cc,YZcn), (Xal,Cc,YZco),(Xal,Cc,YZcp),(Xai,Cc,YZcq),(Xal,Cc,YZcr),(Xal,Cc,YZcs),(Xal, Cc,YZct),(Xal,Cc,YZcu),(Xal,Cc,YZcv),(Xal,Cc,YZcw),(Xal,Cc,YZcx),(Xal,Cc,YZcy), (Xal,Cc,YZcz),(Xal,Cc,YZda),(Xal,Cc,YZdb),(Xal,Cc,YZdc),(Xal,Cc,YZdd),(Xal, Cc,YZde),(Xal,Cc,YZdf),(Xal,Cc,YZdg),(Xal,Cc,YZdh),(Xal,Cc,YZdi),(Xal,Cc,YZdj), (Xal,Cc,YZdk),(Xal,Cc,YZdl),(Xal,Cc,YZdm),(Xal,Cc,YZdn),(Xal,Cc,YZdo),(Xal,Cc, YZdp),(Xal,Cc,YZdq),(Xal,Cc,YZdr),(Xal,Cc,YZds),(Xal,Cc,Yzdt),(Xal,Cc,YZdu),(Xal, Cc,YZdv),(Xal,Cc,YZdw),(Xal,Cc,YZdx),(Xal,Cc,YZdy),(Xal,Cc,YZdz),(Xal,Cc, YZea),(Xal,Cc,YZeb),(Xal,Cc,YZec),(Xal,Cc,YZed),(Xal,Cc,YZee),(Xal,Cc,YZef),(Xal, Cc,YZeg),(Xal,Cc,YZeh),(Xal,Cc,YZei),(Xal,Cc,YZej),(Xal,Cc,YZek),(Xal,Cc,YZel), (Xal,Cc,YZem),(Xal,Cc,YZen),(Xal,Cc,YZeo),(Xal,Cc,YZep),(Xal,Cc,YZeq),(Xal, Cc,YZer),(Xal,Cc,YZes),(Xal,Cc,YZet),(Xal,Cc,YZeu),(Xal,Cc,YZev),(Xal,Cc,YZew), (Xal,Cc,YZex),(Xal,Cc,YZey),(Xal,Cc,YZez),(Xal,Cc,YZfa),(Xal,Cc,YZfb),(Xal,Cc, YZfc),(Xal,Cc,YZfd),(Xal,Cc,YZfe),(Xal,Cc,YZff),(Xal,Cc,YZfg),(Xal,Cc,YZfh),(Xal, Cc,YZfi),(Xal,Cc,YZfj),(Xal,Cc,YZfk),(Xal,Cc,YZfl),(Xal,Cc,YZfm),(Xal,Cc,YZfn),(Xal, Cc,YZfo),(Xal,Cc,YZfp),(Xal,Cc,YZfq),(Xal,Cc,YZfr),(Xal,Cc,YZfs),(Xal,Cc,YZft), (Xal,Cc,YZfu),(Xal,Cc,YZfv),(Xal,Cc,YZfw),(Xal,Cc,YZfx),(Xal,Cc,YZfy),(Xal,Cc, YZfz),(Xal,Cc,YZga),(Xal,Cc,YZgb),(Xal,Cc,YZgc),(Xal,Cc,YZgd),(Xal,Cc,YZge), (Xal,Cc,YZgf),(Xal,Cc,YZgg),(Xal,Cc,YZgh),(Xal,Cc,YZgi),(Xal,Cc,YZgj),(Xal,Cc, YZgk),(Xal,Cc,YZgl),(Xal,Cc,YZgm),(Xal,Cc,YZgn),(Xal,Cc,YZgo),(Xal,Cc,YZgp),(Xal, Cc,YZgq),(Xal,Cc,YZgr),(Xal,Cc,YZgs),(Xal,Cc,YZgt),(Xal,Cd,YZa),(Xal,Cd,YZb), (Xal,Cd,YZc),(Xal,Cd,YZd),(Xal,Cd,YZe),(Xal,Cd,YZf),(Xal,Cd,YZg),(Xal,Cd,YZh), (Xal,Cd,YZi),(Xal,Cd,YZj),(Xal,Cd,YZk),(Xal,Cd,YZl),(Xal,Cd,YZm),(Xal,Cd,YZn), (Xal,Cd,YZo),(Xal,Cd,YZp),(Xal,Cd,YZq),(Xal,Cd,YZr),(Xal,Cd,YZs),(Xal,Cd,YZt), (Xal,Cd,YZu),(Xal,Cd,YZv),(Xal,Cd,YZw),(Xal,Cd,YZx),(Xal,Cd,YZy),(Xal,Cd,YZz), (Xal,Cd,YZaa),(Xal,Cd,YZab),(Xal,Cd,YZac),(Xal,Cd,YZad),(Xal,Cd,YZae),(Xal, Cd,YZaf),(Xal,Cd,YZag),(Xal,Cd,YZah),(Xal,Cd,YZai),(Xal,Cd,YZaj),(Xal,Cd,YZak), (Xal,Cd,YZal),(Xal,Cd,YZam),(Xal,Cd,YZan),(Xal,Cd,YZao),(Xal,Cd,YZap),(Xal,Cd, YZaq),(Xal,Cd,YZar),(Xal,Cd,YZas),(Xal,Cd,YZat),(Xal,Cd,YZau),(Xal,Cd,YZav),(Xal, Cd,YZaw),(Xal,Cd,YZax),(Xal,Cd,YZay),(Xal,Cd,YZaz),(Xal,Cd,YZba),(Xal,Cd, YZbb),(Xal,Cd,YZbc),(Xal,Cd,YZbd),(Xal,Cd,YZbe),(Xal,Cd,YZbf),(Xal,Cd,YZbg),(Xal, Cd,YZbh),(Xal,Cd,YZbi),(Xal,Cd,YZbj),(Xal,Cd,YZbk),(Xal,Cd,YZbl),(Xal,Cd,YZbm), (Xal,Cd,YZbn),(Xal,Cd,YZbo),(Xal,Cd,YZbp),(Xal,Cd,YZbq),(Xal,Cd,YZbr),(Xal, Cd,YZbs),(Xal,Cd,YZbt),(Xal,Cd,YZbu),(Xal,Cd,YZbv),(Xal,Cd,YZbw),(Xal,Cd, YZbx),(Xal,Cd,YZby),(Xal,Cd,YZbz),(Xal,Cd,YZca),(Xal,Cd,YZcb),(Xal,Cd,YZcc),(Xal, Cd,YZcd),(Xal,Cd,YZce),(Xal,Cd,YZcf),(Xal,Cd,YZcg),(Xal,Cd,YZch),(Xal,Cd, YZci),(Xal,Cd,YZcj),(Xal,Cd,YZck),(Xal,Cd,YZcl),(Xal,Cd,YZcm),(Xal,Cd,YZcn),(Xal, Cd,YZco),(Xal,Cd,YZcp),(Xal,Cd,YZcq),(Xal,Cd,YZcr),(Xal,Cd,YZcs),(Xal,Cd, YZct),(Xal,Cd,YZcu),(Xal,Cd,YZcv),(Xal,Cd,YZcw),(Xal,Cd,YZcx),(Xal,Cd,YZcy),(Xal, Cd,YZcz),(Xal,Cd,YZda),(Xal,Cd,YZdb),(Xal,Cd,YZdc),(Xal,Cd,YZdd),(Xal,Cd, YZde),(Xal,Cd,YZdf),(Xal,Cd,YZdg),(Xal,Cd,YZdh),(Xal,Cd,YZdi),(Xal,Cd,YZdj),(Xal, Cd,YZdk),(Xal,Cd,YZdl),(Xal,Cd,YZdm),(Xal,Cd,YZdn),(Xal,Cd,YZdo),(Xal,Cd, YZdp),(Xal,Cd,YZdq),(Xal,Cd,YZdr),(Xal,Cd,YZds),(Xal,Cd,Yzdt),(Xal,Cd,YZdu),(Xal, Cd,YZdv),(Xal,Cd,YZdw),(Xal,Cd,YZdx),(Xal,Cd,YZdy),(Xal,Cd,YZdz),(Xal,Cd, YZea),(Xal,Cd,YZeb),(Xal,Cd,YZec),(Xal,Cd,YZed),(Xal,Cd,YZee),(Xal,Cd,YZef),(Xal, Cd,YZeg),(Xal,Cd,YZeh),(Xal,Cd,YZei),(Xal,Cd,YZej),(Xal,Cd,YZek),(Xal,Cd,YZel), (Xal,Cd,YZem),(Xal,Cd,YZen),(Xal,Cd,YZeo),(Xal,Cd,YZep),(Xal,Cd,YZeq),(Xal, Cd,YZer),(Xal,Cd,YZes),(Xal,Cd,YZet),(Xal,Cd,YZeu),(Xal,Cd,YZev),(Xal,Cd,YZew), (Xal,Cd,YZex),(Xal,Cd,YZey),(Xal,Cd,YZez),(Xal,Cd,YZfa),(Xal,Cd,YZfb),(Xal, Cd,YZfc),(Xal,Cd,YZfd),(Xal,Cd,YZfe),(Xal,Cd,YZff),(Xal,Cd,YZfg),(Xal,Cd,YZfh), (Xal,Cd,YZfi),(Xal,Cd,YZfj),(Xal,Cd,YZfk),(Xal,Cd,YZfl),(Xal,Cd,YZfm),(Xal,Cd,YZfn), (Xal,Cd,YZfo),(Xal,Cd,YZfp),(Xal,Cd,YZfq),(Xal,Cd,YZfr),(Xal,Cd,YZfs),(Xal, Cd,YZft),(Xal,Cd,YZfu),(Xal,Cd,YZfv),(Xal,Cd,YZfw),(Xal,Cd,YZfx),(Xal,Cd,YZfy), (Xal,Cd,YZfz),(Xal,Cd,YZga),(Xal,Cd,YZgb),(Xal,Cd,YZgc),(Xal,Cd,YZgd),(Xal,Cd, YZge),(Xal,Cd,YZgf),(Xal,Cd,YZgg),(Xal,Cd,YZgh),(Xal,Cd,YZgi),(Xal,Cd,YZgj),(Xal, Cd,YZgk),(Xal,Cd,YZgl),(Xal,Cd,YZgm),(Xal,Cd,YZgn),(Xal,Cd,YZgo),(Xal,Cd,YZgp), (Xal,Cd,YZgq),(Xal,Cd,YZgr),(Xal,Cd,YZgs),(Xal,Cd,YZgt),(Xal,Ce,YZa),(Xal, Ce,YZb),(Xal,Ce,YZc),(Xal,Ce,YZd),(Xal,Ce,YZe),(Xal,Ce,YZf),(Xal,Ce,YZg),(Xal, Ce,YZh),(Xal,Ce,YZi),(Xal,Ce,YZj),(Xal,Ce,YZk),(Xal,Ce,YZl),(Xal,Ce,YZm),(Xal, Ce,YZn),(Xal,Ce,YZo),(Xal,Ce,YZp),(Xal,Ce,YZq),(Xal,Ce,YZr),(Xal,Ce,YZs),(Xal, Ce,YZt),(Xal,Ce,YZu),(Xal,Ce,YZv),(Xal,Ce,YZw),(Xal,Ce,YZx),(Xal,Ce,YZy),(Xal, Ce,YZz),(Xal,Ce,YZaa),(Xal,Ce,YZab),(Xal,Ce,YZac),(Xal,Ce,YZad),(Xal,Ce,YZae), (Xal,Ce,YZaf),(Xal,Ce,YZag),(Xal,Ce,YZah),(Xal,Ce,YZai),(Xal,Ce,YZaj),(Xal,Ce,YZak), (Xal,Ce,YZal),(Xal,Ce,YZam),(Xal,Ce,YZan),(Xal,Ce,YZao),(Xal,Ce,YZap),(Xal, Ce,YZaq),(Xal,Ce,YZar),(Xal,Ce,YZas),(Xal,Ce,YZat),(Xal,Ce,YZau),(Xal,Ce,YZav), (Xal,Ce,YZaw),(Xal,Ce,YZax),(Xal,Ce,YZay),(Xal,Ce,YZaz),(Xal,Ce,YZba),(Xal,Ce, YZbb),(Xal,Ce,YZbc),(Xal,Ce,YZbd),(Xal,Ce,YZbe),(Xal,Ce,YZbf),(Xal,Ce,YZbg), (Xal,Ce,YZbh),(Xal,Ce,YZbi),(Xal,Ce,YZbj),(Xal,Ce,YZbk),(Xal,Ce,YZbl),(Xal,Ce,YZbm), (Xal,Ce,YZbn),(Xal,Ce,YZbo),(Xal,Ce,YZbp),(Xal,Ce,YZbq),(Xal,Ce,YZbr),(Xal, Ce,YZbs),(Xal,Ce,YZbt),(Xal,Ce,YZbu),(Xal,Ce,YZbv),(Xal,Ce,YZbw),(Xal,Ce,YZbx), (Xal,Ce,YZby),(Xal,Ce,YZbz),(Xal,Ce,YZca),(Xal,Ce,YZcb),(Xal,Ce,YZcc),(Xal, Ce,YZcd),(Xal,Ce,YZce),(Xal,Ce,YZcf),(Xal,Ce,YZcg),(Xal,Ce,YZch),(Xal,Ce,YZci), (Xal,Ce,YZcj),(Xal,Ce,YZck),(Xal,Ce,YZcl),(Xal,Ce,YZcm),(Xal,Ce,YZcn),(Xal,Ce, YZco),(Xal,Ce,YZcp),(Xal,Ce,YZcq),(Xal,Ce,YZcr),(Xal,Ce,YZcs),(Xal,Ce,YZct),(Xal, Ce,YZcu),(Xal,Ce,YZcv),(Xal,Ce,YZcw),(Xal,Ce,YZcx),(Xal,Ce,YZcy),(Xal,Ce,YZcz), (Xal,Ce,YZda),(Xal,Ce,YZdb),(Xal,Ce,YZdc),(Xal,Ce,YZdd),(Xal,Ce,YZde),(Xal, Ce,YZdf),(Xal,Ce,YZdg),(Xal,Ce,YZdh),(Xal,Ce,YZdi),(Xal,Ce,YZdj),(Xal,Ce,YZdk), (Xal,Ce,YZdl),(Xal,Cd,YZdm),(Xal,Ce,YZdn),(Xal,Ce,YZdo),(Xal,Ce,YZdp),(Xal, Ce,YZdq),(Xal,Ce,YZdr),(Xal,Ce,YZds),(Xal,Ce,Yzdt),(Xal,Ce,YZdu),(Xal,Ce,YZdv), (Xal,Ce,YZdw),(Xal,Ce,YZdx),(Xal,Ce,YZdy),(Xal,Ce,YZdz),(Xal,Ce,YZea),(Xal,Ce, YZeb),(Xal,Ce,YZec),(Xal,Ce,YZed),(Xal,Ce,YZee),(Xal,Ce,YZef),(Xal,Ce,YZeg),(Xal, Ce,YZeh),(Xal,Ce,YZei),(Xal,Ce,YZej),(Xal,Ce,YZek),(Xal,Ce,YZel),(Xal,Ce,YZem), (Xal,Ce,YZen),(Xal,Ce,YZeo),(Xal,Ce,YZep),(Xal,Ce,YZeq),(Xal,Ce,YZer),(Xal, Ce,YZes),(Xal,Ce,YZet),(Xal,Ce,YZeu),(Xal,Ce,YZev),(Xal,Ce,YZew),(Xal,Ce,YZex), (Xal,Ce,YZey),(Xal,Ce,YZez),(Xal,Ce,YZfa),(Xal,Ce,YZfb),(Xal,Ce,YZfc),(Xal,Ce, YZfd),(Xal,Ce,YZfe),(Xal,Ce,YZff),(Xal,Ce,YZfg),(Xal,Ce,YZfh),(Xal,Ce,YZfi),(Xal, Ce,YZfj),(Xal,Ce,YZfk),(Xal,Ce,YZfl),(Xal,Ce,YZfm),(Xal,Ce,YZfn),(Xal,Ce,YZfo), (Xal,Ce,YZfp),(Xal,Ce,YZfq),(Xal,Ce,YZfr),(Xal,Ce,YZfs),(Xal,Ce,YZft),(Xal,Ce,YZfu), (Xal,Ce,YZfv),(Xal,Ce,YZfw),(Xal,Ce,YZfx),(Xal,Ce,YZfy),(Xal,Ce,YZfz),(Xal,Ce, YZga),(Xal,Ce,YZgb),(Xal,Ce,YZgc),(Xal,Ce,YZgd),(Xal,Ce,YZge),(Xal,Ce,YZgf), (Xal,Ce,YZgg),(Xal,Ce,YZgh),(Xal,Ce,YZgi),(Xal,Ce,YZgj),(Xal,Ce,YZgk),(Xal,Ce, YZgl),(Xal,Ce,YZgm),(Xal,Ce,YZgn),(Xal,Ce,YZgo),(Xal,Ce,YZgp),(Xal,Ce,YZgq),(Xal, Ce,YZgr),(Xal,Ce,YZgs),(Xal,Ce,YZgt),(Xal,Cf,YZa),(Xal,Cf,YZb),(Xal,Cf,YZc), (Xal,Cf,YZd),(Xal,Cf,YZe),(Xal,Cf,YZf),(Xal,Cf,YZg),(Xal,Cf,YZh),(Xal,Cf,YZi),(Xal, Cf,YZj),(Xal,Cf,YZk),(Xal,Cf,YZl),(Xal,Cf,YZm),(Xal,Cf,YZn),(Xal,Cf,YZo),(Xal, Cf,YZp),(Xal,Cf,YZq),(Xal,Cf,YZr),(Xal,Cf,YZs),(Xal,Cf,YZt),(Xal,Cf,YZu),(Xal, Cf,YZv),(Xal,Cf,YZw),(Xal,Cf,YZx),(Xal,Cf,YZy),(Xal,Cf,YZz),(Xal,Cf,YZaa),(Xal, Cf,YZab),(Xal,Cf,YZac),(Xal,Cf,YZad),(Xal,Cf,YZae),(Xal,Cf,YZaf),(Xal,Cf,YZag),(Xal, Cf,YZah),(Xal,Cf,YZai),(Xal,Cf,YZaj),(Xal,Cf,YZak),(Xal,Cf,YZal),(Xal,Cf,YZam), (Xal,Cf,YZan),(Xal,Cf,YZao),(Xal,Cf,YZap),(Xal,Cf,YZaq),(Xal,Cf,YZar),(Xal,Cf, YZas),(Xal,Cf,YZat),(Xal,Cf,YZau),(Xal,Cf,YZav),(Xal,Cf,YZaw),(Xal,Cf,YZax),(Xal, Cf,YZay),(Xal,Cf,YZaz),(Xal,Cf,YZba),(Xal,Cf,YZbb),(Xal,Cf,YZbc),(Xal,Cf,YZbd), (Xal,Cf,YZbe),(Xal,Cf,YZbf),(Xal,Cf,YZbg),(Xal,Cf,YZbh),(Xal,Cf,YZbi),(Xal,Cf, YZbj),(Xal,Cf,YZbk),(Xal,Cf,YZbl),(Xal,Cf,YZbm),(Xal,Cf,YZbn),(Xal,Cf,YZbo),(Xal, Cf,YZbp),(Xal,Cf,YZbq),(Xal,Cf,YZbr),(Xal,Cf,YZbs),(Xal,Cf,YZbt),(Xal,Cf,YZbu), (Xal,Cf,YZbv),(Xal,Cf,YZbw),(Xal,Cf,YZbx),(Xal,Cf,YZby),(Xal,Cf,YZbz),(Xal,Cf, YZca),(Xal,Cf,YZcb),(Xal,Cf,YZcc),(Xal,Cf,YZcd),(Xal,Cf,YZce),(Xal,Cf,YZcf),(Xal, Cf,YZcg),(Xal,Cf,YZch),(Xal,Cf,YZci),(Xal,Cf,YZcj),(Xal,Cf,YZck),(Xal,Cf,YZcl), (Xal,Cf,YZcm),(Xal,Cf,YZcn),(Xal,Cf,YZco),(Xal,Cf,YZcp),(Xal,Cf,YZcq),(Xal,Cf, YZcr),(Xal,Cf,YZcs),(Xal,Cf,YZct),(Xal,Cf,YZcu),(Xal,Cf,YZcv),(Xal,Cf,YZcw),(Xal, Cf,YZcx),(Xal,Cf,YZcy),(Xal,Cf,YZcz),(Xal,Cf,YZda),(Xal,Cf,YZdb),(Xal,Cf,YZdc), (Xal,Cf,YZdd),(Xal,Cf,YZde),(Xal,Cf,YZdf),(Xal,Cf,YZdg),(Xal,Cf,YZdh),(Xal,Cf, YZdi),(Xal,Cf,YZdj),(Xal,Cf,YZdk),(Xal,Cf,YZdl),(Xal,Cf,YZdm),(Xal,Cf,YZdn),(Xal, Cf,YZdo),(Xal,Cf,YZdp),(Xal,Cf,YZdq),(Xal,Cf,YZdr),(Xal,Cf,YZds),(Xal,Cf,Yzdt), (Xal,Cf,YZdu),(Xal,Cf,YZdv),(Xal,Cf,YZdw),(Xal,Cf,YZdx),(Xal,Cf,YZdy),(Xal,Cf, YZdz),(Xal,Cf,YZea),(Xal,Cf,YZeb),(Xal,Cf,YZec),(Xal,Cf,YZed),(Xal,Cf,YZee),(Xal, Cf,YZef),(Xal,Cf,YZeg),(Xal,Cf,YZeh),(Xal,Cf,YZei),(Xal,Cf,YZej),(Xal,Cf,YZek), (Xal,Cf,YZel),(Xal,Cf,YZem),(Xal,Cf,YZen),(Xal,Cf,YZeo),(Xal,Cf,YZep),(Xal,Cf,YZeq), (Xal,Cf,YZer),(Xal,Cf,YZes),(Xal,Cf,YZet),(Xal,Cf,YZeu),(Xal,Cf,YZev),(Xal, Cf,YZew),(Xal,Cf,YZex),(Xal,Cf,YZey),(Xal,Cf,YZez),(Xal,Cf,YZfa),(Xal,Cf,YZfb), (Xal,Cf,YZfc),(Xal,Cf,YZfd),(Xal,Cf,YZfe),(Xal,Cf,YZff),(Xal,Cf,YZfg),(Xal,Cf,YZfh), (Xal,Cf,YZfi),(Xal,Cf,YZfj),(Xal,Cf,YZfk),(Xal,Cf,YZfl),(Xal,Cf,YZfm),(Xal,Cf,YZfn), (Xal,Cf,YZfo),(Xal,Cf,YZfp),(Xal,Cf,YZfq),(Xal,Cf,YZfr),(Xal,Cf,YZfs),(Xal,Cf, YZft),(Xal,Cf,YZfu),(Xal,Cf,YZfv),(Xal,Cf,YZfw),(Xal,Cf,YZfx),(Xal,Cf,YZfy),(Xal, Cf,YZfz),(Xal,Cf,YZga),(Xal,Cf,YZgb),(Xal,Cf,YZgc),(Xal,Cf,YZgd),(Xal,Cf,YZge), (Xal,Cf,YZgf),(Xal,Cf,YZgg),(Xal,Cf,YZgh),(Xal,Cf,YZgi),(Xal,Cf,YZgj),(Xal,Cf,YZgk), (Xal,Cf,YZgl),(Xal,Cf,YZgm),(Xal,Cf,YZgn),(Xal,Cf,YZgo),(Xal,Cf,YZgp),(Xal, Cf,YZgq),(Xal,Cf,YZgr),(Xal,Cf,YZgs),(Xal,Cf,YZgt), (Xam,Ca,YZa),(Xam,Ca,YZb),(Xam,Ca,YZc),(Xam,Ca,YZd),(Xam,Ca,YZe),(Xam,Ca, YZf),(Xam,Ca,YZg),(Xam,Ca,YZh),(Xam,Ca,YZi),(Xam,Ca,YZj),(Xam,Ca,YZk),(Xam, Ca,YZl),(Xam,Ca,YZm),(Xam,Ca,YZn),(Xam,Ca,YZo),(Xam,Ca,YZp),(Xam,Ca, YZq),(Xam,Ca,YZr),(Xam,Ca,YZs),(Xam,Ca,YZt),(Xam,Ca,YZu),(Xam,Ca,YZv),(Xam, Ca,YZw),(Xam,Ca,YZx),(Xam,Ca,YZy),(Xam,Ca,YZz),(Xam,Ca,YZaa),(Xam,Ca, YZab),(Xam,Ca,YZac),(Xam,Ca,YZad),(Xam,Ca,YZae),(Xam,Ca,YZaf),(Xam,Ca,YZag), (Xam,Ca,YZah),(Xam,Ca,YZai),(Xam,Ca,YZaj),(Xam,Ca,YZak),(Xam,Ca,YZal),(Xam, Ca,YZam),(Xam,Ca,YZan),(Xam,Ca,YZao),(Xam,Ca,YZap),(Xam,Ca,YZaq),(Xam, Ca,YZar),(Xam,Ca,YZas),(Xam,Ca,YZat),(Xam,Ca,YZau),(Xam,Ca,YZav),(Xam, Ca,YZaw),(Xam,Ca,YZax),(Xam,Ca,YZay),(Xam,Ca,YZaz),(Xam,Ca,YZba),(Xam,Ca, YZbb),(Xam,Ca,YZbc),(Xam,Ca,YZbd),(Xam,Ca,YZbe),(Xam,Ca,YZbf),(Xam,Ca, YZbg),(Xam,Ca,YZbh),(Xam,Ca,YZbi),(Xam,Ca,YZbj),(Xam,Ca,YZbk),(Xam,Ca,YZbl), (Xam,Ca,YZbm),(Xam,Ca,YZbn),(Xam,Ca,YZbo),(Xam,Ca,YZbp),(Xam,Ca,YZbq), (Xam,Ca,YZbr),(Xam,Ca,YZbs),(Xam,Ca,YZbt),(Xam,Ca,YZbu),(Xam,Ca,YZbv),(Xam, Ca,YZbw),(Xam,Ca,YZbx),(Xam,Ca,YZby),(Xam,Ca,YZbz),(Xam,Ca,YZca),(Xam, Ca,YZcb),(Xam,Ca,YZcc),(Xam,Ca,YZcd),(Xam,Ca,YZce),(Xam,Ca,YZcf),(Xam, Ca,YZcg),(Xam,Ca,YZch),(Xam,Ca,YZci),(Xam,Ca,YZcj),(Xam,Ca,YZck),(Xam,Ca, YZcl),(Xam,Ca,YZcm),(Xam,Ca,YZcn),(Xam,Ca,YZco),(Xam,Ca,YZcp),(Xam,Ca,YZcq), (Xam,Ca,YZcr),(Xam,Ca,YZcs),(Xam,Ca,YZct),(Xam,Ca,YZcu),(Xam,Ca,YZcv), (Xam,Ca,YZcw),(Xam,Ca,YZcx),(Xam,Ca,YZcy),(Xam,Ca,YZcz),(Xam,Ca,YZda),(Xam, Ca,YZdb),(Xam,Ca,YZdc),(Xam,Ca,YZdd),(Xam,Ca,YZde),(Xam,Ca,YZdf),(Xam, Ca,YZdg),(Xam,Ca,YZdh),(Xam,Ca,YZdi),(Xam,Ca,YZdj),(Xam,Ca,YZdk),(Xam, Ca,YZdl),(Xam,Ca,YZdm),(Xam,Ca,YZdn),(Xam,Ca,YZdo),(Xam,Ca,YZdp),(Xam,Ca, YZdq),(Xam,Ca,YZdr),(Xam,Ca,YZds),(Xam,Ca,Yzdt),(Xam,Ca,YZdu),(Xam,Ca,YZdv), (Xam,Ca,YZdw),(Xam,Ca,YZdx),(Xam,Ca,YZdy),(Xam,Ca,YZdz),(Xam,Ca,YZea), (Xam,Ca,YZeb),(Xam,Ca,YZec),(Xam,Ca,YZed),(Xam,Ca,YZee),(Xam,Ca,YZef), (Xam,Ca,YZeg),(Xam,Ca,YZeh),(Xam,Ca,YZei),(Xam,Ca,YZej),(Xam,Ca,YZek),(Xam, Ca,YZel),(Xam,Ca,YZem),(Xam,Ca,YZen),(Xam,Ca,YZeo),(Xam,Ca,YZep),(Xam, Ca,YZeq),(Xam,Ca,YZer),(Xam,Ca,YZes),(Xam,Ca,YZet),(Xam,Ca,YZeu),(Xam,Ca, YZev),(Xam,Ca,YZew),(Xam,Ca,YZex),(Xam,Ca,YZey),(Xam,Ca,YZez),(Xam,Ca,YZfa), (Xam,Ca,YZfb),(Xam,Ca,YZfc),(Xam,Ca,YZfd),(Xam,Ca,YZfe),(Xam,Ca,YZff), (Xam,Ca,YZfg),(Xam,Ca,YZfh),(Xam,Ca,YZfi),(Xam,Ca,YZfj),(Xam,Ca,YZfk),(Xam, Ca,YZfl),(Xam,Ca,YZfm),(Xam,Ca,YZfn),(Xam,Ca,YZfo),(Xam,Ca,YZfp),(Xam,Ca, YZfq),(Xam,Ca,YZfr),(Xam,Ca,YZfs),(Xam,Ca,YZft),(Xam,Ca,YZfu),(Xam,Ca,YZfv), (Xam,Ca,YZfw),(Xam,Ca,YZfx),(Xam,Ca,YZfy),(Xam,Ca,YZfz),(Xam,Ca,YZga), (Xam,Ca,YZgb),(Xam,Ca,YZgc),(Xam,Ca,YZgd),(Xam,Ca,YZge),(Xam,Ca,YZgf),(Xam, Ca,YZgg),(Xam,Ca,YZgh),(Xam,Ca,YZgi),(Xam,Ca,YZgj),(Xam,Ca,YZgk),(Xam, Ca,YZgl),(Xam,Ca,YZgm),(Xam,Ca,YZgn),(Xam,Ca,YZgo),(Xam,Ca,YZgp),(Xam,Ca, YZgq),(Xam,Ca,YZgr),(Xam,Ca,YZgs),(Xam,Ca,YZgt),(Xam,Cb,YZa),(Xam,Cb,YZb), (Xam,Cb,YZc),(Xam,Cb,YZd),(Xam,Cb,YZe),(Xam,Cb,YZf),(Xam,Cb,YZg),(Xam, Cb,YZh),(Xam,Cb,YZi),(Xam,Cb,YZj),(Xam,Cb,YZk),(Xam,Cb,YZl),(Xam,Cb,YZm), (Xam,Cb,YZn),(Xam,Cb,YZo),(Xam,Cb,YZp),(Xam,Cb,YZq),(Xam,Cb,YZr),(Xam, Cb,YZs),(Xam,Cb,YZt),(Xam,Cb,YZu),(Xam,Cb,YZv),(Xam,Cb,YZw),(Xam,Cb,YZx), (Xam,Cb,YZy),(Xam,Cb,YZz),(Xam,Cb,YZaa),(Xam,Cb,YZab),(Xam,Cb,YZac),(Xam, Cb,YZad),(Xam,Cb,YZae),(Xam,Cb,YZaf),(Xam,Cb,YZag),(Xam,Cb,YZah),(Xam, Cb,YZai),(Xam,Cb,YZaj),(Xam,Cb,YZak),(Xam,Cb,YZal),(Xam,Cb,YZam),(Xam, Cb,YZan),(Xam,Cb,YZao),(Xam,Cb,YZap),(Xam,Cb,YZaq),(Xam,Cb,YZar),(Xam,Cb, YZas),(Xam,Cb,YZat),(Xam,Cb,YZau),(Xam,Cb,YZav),(Xam,Cb,YZaw),(Xam,Cb, YZax),(Xam,Cb,YZay),(Xam,Cb,YZaz),(Xam,Cb,YZba),(Xam,Cb,YZbb),(Xam,Cb,YZbc), (Xam,Cb,YZbd),(Xam,Cb,YZbe),(Xam,Cb,YZbf),(Xam,Cb,YZbg),(Xam,Cb,YZbh), (Xam,Cb,YZbi),(Xam,Cb,YZbj),(Xam,Cb,YZbk),(Xam,Cb,YZbl),(Xam,Cb,YZbm), (Xam,Cb,YZbn),(Xam,Cb,YZbo),(Xam,Cb,YZbp),(Xam,Cb,YZbq),(Xam,Cb,YZbr), (Xam,Cb,YZbs),(Xam,Cb,YZbt),(Xam,Cb,YZbu),(Xam,Cb,YZbv),(Xam,Cb,YZbw),(Xam, Cb,YZbx),(Xam,Cb,YZby),(Xam,Cb,YZbz),(Xam,Cb,YZca),(Xam,Cb,YZcb),(Xam, Cb,YZcc),(Xam,Cb,YZcd),(Xam,Cb,YZce),(Xam,Cb,YZcf),(Xam,Cb,YZcg),(Xam, Cb,YZch),(Xam,Cb,YZci),(Xam,Cb,YZcj),(Xam,Cb,YZck),(Xam,Cb,YZcl),(Xam,Cb, YZcm),(Xam,Cb,YZcn),(Xam,Cb,YZco),(Xam,Cb,YZcp),(Xam,Cb,YZcq),(Xam,Cb, YZcr),(Xam,Cb,YZcs),(Xam,Cb,YZct),(Xam,Cb,YZcu),(Xam,Cb,YZcv),(Xam,Cb,YZcw), (Xam,Cb,YZcx),(Xam,Cb,YZcy),(Xam,Cb,YZcz),(Xam,Cb,YZda),(Xam,Cb,YZdb), (Xam,Cb,YZdc),(Xam,Cb,YZdd),(Xam,Cb,YZde),(Xam,Cb,YZdf),(Xam,Cb,YZdg), (Xam,Cb,YZdh),(Xam,Cb,YZdi),(Xam,Cb,YZdj),(Xam,Cb,YZdk),(Xam,Cb,YZdl),(Xam, Cb,YZdm),(Xam,Cb,YZdn),(Xam,Cb,YZdo),(Xam,Cb,YZdp),(Xam,Cb,YZdq),(Xam, Cb,YZdr),(Xam,Cb,YZds),(Xam,Cb,Yzdt),(Xam,Cb,YZdu),(Xam,Cb,YZdv),(Xam, Cb,YZdw),(Xam,Cb,YZdx),(Xam,Cb,YZdy),(Xam,Cb,YZdz),(Xam,Cb,YZea),(Xam, Cb,YZeb),(Xam,Cb,YZec),(Xam,Cb,YZed),(Xam,Cb,YZee),(Xam,Cb,YZef),(Xam,Cb, YZeg),(Xam,Cb,YZeh),(Xam,Cb,YZei),(Xam,Cb,YZej),(Xam,Cb,YZek),(Xam,Cb,YZel), (Xam,Cb,YZem),(Xam,Cb,YZen),(Xam,Cb,YZeo),(Xam,Cb,YZep),(Xam,Cb,YZeq), (Xam,Cb,YZer),(Xam,Cb,YZes),(Xam,Cb,YZet),(Xam,Cb,YZeu),(Xam,Cb,YZev), (Xam,Cb,YZew),(Xam,Cb,YZex),(Xam,Cb,YZey),(Xam,Cb,YZez),(Xam,Cb,YZfa),(Xam, Cb,YZfb),(Xam,Cb,YZfc),(Xam,Cb,YZfd),(Xam,Cb,YZfe),(Xam,Cb,YZff),(Xam, Cb,YZfg),(Xam,Cb,YZfh),(Xam,Cb,YZfi),(Xam,Cb,YZfj),(Xam,Cb,YZfk),(Xam,Cb, YZfl),(Xam,Cb,YZfm),(Xam,Cb,YZfn),(Xam,Cb,YZfo),(Xam,Cb,YZfp),(Xam,Cb,YZfq), (Xam,Cb,YZfr),(Xam,Cb,YZfs),(Xam,Cb,YZft),(Xam,Cb,YZfu),(Xam,Cb,YZfv), (Xam,Cb,YZfw),(Xam,Cb,YZfx),(Xam,Cb,YZfy),(Xam,Cb,YZfz),(Xam,Cb,YZga),(Xam, Cb,YZgb),(Xam,Cb,YZgc),(Xam,Cb,YZgd),(Xam,Cb,YZge),(Xam,Cb,YZgf),(Xam, Cb,YZgg),(Xam,Cb,YZgh),(Xam,Cb,YZgi),(Xam,Cb,YZgj),(Xam,Cb,YZgk),(Xam, Cb,YZgl),(Xam,Cb,YZgm),(Xam,Cb,YZgn),(Xam,Cb,YZgo),(Xam,Cb,YZgp),(Xam, Cb,YZgq),(Xam,Cb,YZgr),(Xam,Cb,YZgs),(Xam,Cb,YZgt),(Xam,Cc,YZa),(Xam,Cc, YZb),(Xam,Cc,YZc),(Xam,Cc,YZd),(Xam,Cc,YZe),(Xam,Cc,YZf),(Xam,Cc,YZg),(Xam, Cc,YZh),(Xam,Cc,YZi),(Xam,Cc,YZj),(Xam,Cc,YZk),(Xam,Cc,YZl),(Xam,Cc,YZm), (Xam,Cc,YZn),(Xam,Cc,YZo),(Xam,Cc,YZp),(Xam,Cc,YZq),(Xam,Cc,YZr),(Xam, Cc,YZs),(Xam,Cc,YZt),(Xam,Cc,YZu),(Xam,Cc,YZv),(Xam,Cc,YZw),(Xam,Cc,YZx), (Xam,Cc,YZy),(Xam,Cc,YZz),(Xam,Cc,YZaa),(Xam,Cc,YZab),(Xam,Cc,YZac),(Xam, Cc,YZad),(Xam,Cc,YZae),(Xam,Cc,YZaf),(Xam,Cc,YZag),(Xam,Cc,YZah),(Xam, Cc,YZai),(Xam,Cc,YZaj),(Xam,Cc,YZak),(Xam,Cc,YZal),(Xam,Cc,YZam),(Xam, Cc,YZan),(Xam,Cc,YZao),(Xam,Cc,YZap),(Xam,Cc,YZaq),(Xam,Cc,YZar),(Xam,Cc, YZas),(Xam,Cc,YZat),(Xam,Cc,YZau),(Xam,Cc,YZav),(Xam,Cc,YZaw),(Xam,Cc,YZax), (Xam,Cc,YZay),(Xam,Cc,YZaz),(Xam,Cc,YZba),(Xam,Cc,YZbb),(Xam,Cc,YZbc), (Xam,Cc,YZbd),(Xam,Cc,YZbe),(Xam,Cc,YZbf),(Xam,Cc,YZbg),(Xam,Cc,YZbh),(Xam, Cc,YZbi),(Xam,Cc,YZbj),(Xam,Cc,YZbk),(Xam,Cc,YZbl),(Xam,Cc,YZbm),(Xam, Cc,YZbn),(Xam,Cc,YZbo),(Xam,Cc,YZbp),(Xam,Cc,YZbq),(Xam,Cc,YZbr),(Xam, Cc,YZbs),(Xam,Cc,YZbt),(Xam,Cc,YZbu),(Xam,Cc,YZbv),(Xam,Cc,YZbw),(Xam,Cc, YZbx),(Xam,Cc,YZby),(Xam,Cc,YZbz),(Xam,Cc,YZca),(Xam,Cc,YZcb),(Xam,Cc, YZcc),(Xam,Cc,YZcd),(Xam,Cc,YZce),(Xam,Cc,YZcf),(Xam,Cc,YZcg),(Xam,Cc,YZch), (Xam,Cc,YZci),(Xam,Cc,YZcj),(Xam,Cc,YZck),(Xam,Cc,YZcl),(Xam,Cc,YZcm), (Xam,Cc,YZcn),(Xam,Cc,YZco),(Xam,Cc,YZcp),(Xam,Cc,YZcq),(Xam,Cc,YZcr),(Xam, Cc,YZcs),(Xam,Cc,YZct),(Xam,Cc,YZcu),(Xam,Cc,YZcv),(Xam,Cc,YZcw),(Xam, Cc,YZcx),(Xam,Cc,YZcy),(Xam,Cc,YZcz),(Xam,Cc,YZda),(Xam,Cc,YZdb),(Xam,Cc, YZdc),(Xam,Cc,YZdd),(Xam,Cc,YZde),(Xam,Cc,YZdf),(Xam,Cc,YZdg),(Xam,Cc, YZdh),(Xam,Cc,YZdi),(Xam,Cc,YZdj),(Xam,Cc,YZdk),(Xam,Cc,YZdl),(Xam,Cc,YZdm), (Xam,Cc,YZdn),(Xam,Cc,YZdo),(Xam,Cc,YZdp),(Xam,Cc,YZdq),(Xam,Cc,YZdr), (Xam,Cc,YZds),(Xam,Cc,Yzdt),(Xam,Cc,YZdu),(Xam,Cc,YZdv),(Xam,Cc,YZdw),(Xam, Cc,YZdx),(Xam,Cc,YZdy),(Xam,Cc,YZdz),(Xam,Cc,YZea),(Xam,Cc,YZeb),(Xam, Cc,YZec),(Xam,Cc,YZed),(Xam,Cc,YZee),(Xam,Cc,YZef),(Xam,Cc,YZeg),(Xam, Cc,YZeh),(Xam,Cc,YZei),(Xam,Cc,YZej),(Xam,Cc,YZek),(Xam,Cc,YZel),(Xam,Cc, YZem),(Xam,Cc,YZen),(Xam,Cc,YZeo),(Xam,Cc,YZep),(Xam,Cc,YZeq),(Xam,Cc,YZer), (Xam,Cc,YZes),(Xam,Cc,YZet),(Xam,Cc,YZeu),(Xam,Cc,YZev),(Xam,Cc,YZew), (Xam,Cc,YZex),(Xam,Cc,YZey),(Xam,Cc,YZez),(Xam,Cc,YZfa),(Xam,Cc,YZfb), (Xam,Cc,YZfc),(Xam,Cc,YZfd),(Xam,Cc,YZfe),(Xam,Cc,YZff),(Xam,Cc,YZfg),(Xam, Cc,YZfh),(Xam,Cc,YZfi),(Xam,Cc,YZfj),(Xam,Cc,YZfk),(Xam,Cc,YZfl),(Xam,Cc, YZfm),(Xam,Cc,YZfn),(Xam,Cc,YZfo),(Xam,Cc,YZfp),(Xam,Cc,YZfq),(Xam,Cc,YZfr), (Xam,Cc,YZfs),(Xam,Cc,YZft),(Xam,Cc,YZfu),(Xam,Cc,YZfv),(Xam,Cc,YZfw),(Xam, Cc,YZfx),(Xam,Cc,YZfy),(Xam,Cc,YZfz),(Xam,Cc,YZga),(Xam,Cc,YZgb),(Xam, Cc,YZgc),(Xam,Cc,YZgd),(Xam,Cc,YZge),(Xam,Cc,YZgf),(Xam,Cc,YZgg),(Xam, Cc,YZgh),(Xam,Cc,YZgi),(Xam,Cc,YZgj),(Xam,Cc,YZgk),(Xam,Cc,YZgl),(Xam,Cc, YZgm),(Xam,Cc,YZgn),(Xam,Cc,YZgo),(Xam,Cc,YZgp),(Xam,Cc,YZgq),(Xam,Cc, YZgr),(Xam,Cc,YZgs),(Xam,Cc,YZgt),(Xam,Cd,YZa),(Xam,Cd,YZb),(Xam,Cd,YZc), (Xam,Cd,YZd),(Xam,Cd,YZe),(Xam,Cd,YZf),(Xam,Cd,YZg),(Xam,Cd,YZh),(Xam,Cd, YZi),(Xam,Cd,YZj),(Xam,Cd,YZk),(Xam,Cd,YZl),(Xam,Cd,YZm),(Xam,Cd,YZn),(Xam, Cd,YZo),(Xam,Cd,YZp),(Xam,Cd,YZq),(Xam,Cd,YZr),(Xam,Cd,YZs),(Xam,Cd, YZt),(Xam,Cd,YZu),(Xam,Cd,YZv),(Xam,Cd,YZw),(Xam,Cd,YZx),(Xam,Cd,YZy),(Xam, Cd,YZz),(Xam,Cd,YZaa),(Xam,Cd,YZab),(Xam,Cd,YZac),(Xam,Cd,YZad),(Xam, Cd,YZae),(Xam,Cd,YZaf),(Xam,Cd,YZag),(Xam,Cd,YZah),(Xam,Cd,YZai),(Xam, Cd,YZaj),(Xam,Cd,YZak),(Xam,Cd,YZal),(Xam,Cd,YZam),(Xam,Cd,YZan),(Xam,Cd, YZao),(Xam,Cd,YZap),(Xam,Cd,YZaq),(Xam,Cd,YZar),(Xam,Cd,YZas),(Xam,Cd, YZat),(Xam,Cd,YZau),(Xam,Cd,YZav),(Xam,Cd,YZaw),(Xam,Cd,YZax),(Xam,Cd,YZay), (Xam,Cd,YZaz),(Xam,Cd,YZba),(Xam,Cd,YZbb),(Xam,Cd,YZbc),(Xam,Cd,YZbd), (Xam,Cd,YZbe),(Xam,Cd,YZbf),(Xam,Cd,YZbg),(Xam,Cd,YZbh),(Xam,Cd,YZbi), (Xam,Cd,YZbj),(Xam,Cd,YZbk),(Xam,Cd,YZbl),(Xam,Cd,YZbm),(Xam,Cd,YZbn), (Xam,Cd,YZbo),(Xam,Cd,YZbp),(Xam,Cd,YZbq),(Xam,Cd,YZbr),(Xam,Cd,YZbs),(Xam, Cd,YZbt),(Xam,Cd,YZbu),(Xam,Cd,YZbv),(Xam,Cd,YZbw),(Xam,Cd,YZbx),(Xam, Cd,YZby),(Xam,Cd,YZbz),(Xam,Cd,YZca),(Xam,Cd,YZcb),(Xam,Cd,YZcc),(Xam, Cd,YZcd),(Xam,Cd,YZce),(Xam,Cd,YZcf),(Xam,Cd,YZcg),(Xam,Cd,YZch),(Xam, Cd,YZci),(Xam,Cd,YZcj),(Xam,Cd,YZck),(Xam,Cd,YZcl),(Xam,Cd,YZcm),(Xam, Cd,YZcn),(Xam,Cd,YZco),(Xam,Cd,YZcp),(Xam,Cd,YZcq),(Xam,Cd,YZcr),(Xam,Cd, YZcs),(Xam,Cd,YZct),(Xam,Cd,YZcu),(Xam,Cd,YZcv),(Xam,Cd,YZcw),(Xam,Cd, YZcx),(Xam,Cd,YZcy),(Xam,Cd,YZcz),(Xam,Cd,YZda),(Xam,Cd,YZdb),(Xam,Cd,YZdc), (Xam,Cd,YZdd),(Xam,Cd,YZde),(Xam,Cd,YZdf),(Xam,Cd,YZdg),(Xam,Cd,YZdh), (Xam,Cd,YZdi),(Xam,Cd,YZdj),(Xam,Cd,YZdk),(Xam,Cd,YZdl),(Xam,Cd,YZdm), (Xam,Cd,YZdn),(Xam,Cd,YZdo),(Xam,Cd,YZdp),(Xam,Cd,YZdq),(Xam,Cd,YZdr), (Xam,Cd,YZds),(Xam,Cd,Yzdt),(Xam,Cd,YZdu),(Xam,Cd,YZdv),(Xam,Cd,YZdw),(Xam, Cd,YZdx),(Xam,Cd,YZdy),(Xam,Cd,YZdz),(Xam,Cd,YZea),(Xam,Cd,YZeb),(Xam, Cd,YZec),(Xam,Cd,YZed),(Xam,Cd,YZee),(Xam,Cd,YZef),(Xam,Cd,YZeg),(Xam, Cd,YZeh),(Xam,Cd,YZei),(Xam,Cd,YZej),(Xam,Cd,YZek),(Xam,Cd,YZel),(Xam,Cd, YZem),(Xam,Cd,YZen),(Xam,Cd,YZeo),(Xam,Cd,YZep),(Xam,Cd,YZeq),(Xam,Cd, YZer),(Xam,Cd,YZes),(Xam,Cd,YZet),(Xam,Cd,YZeu),(Xam,Cd,YZev),(Xam,Cd,YZew), (Xam,Cd,YZex),(Xam,Cd,YZey),(Xam,Cd,YZez),(Xam,Cd,YZfa),(Xam,Cd,YZfb), (Xam,Cd,YZfc),(Xam,Cd,YZfd),(Xam,Cd,YZfe),(Xam,Cd,YZff),(Xam,Cd,YZfg),(Xam, Cd,YZfh),(Xam,Cd,YZfi),(Xam,Cd,YZfj),(Xam,Cd,YZfk),(Xam,Cd,YZfl),(Xam,Cd, YZfm),(Xam,Cd,YZfn),(Xam,Cd,YZfo),(Xam,Cd,YZfp),(Xam,Cd,YZfq),(Xam,Cd, YZfr),(Xam,Cd,YZfs),(Xam,Cd,YZft),(Xam,Cd,YZfu),(Xam,Cd,YZfv),(Xam,Cd,YZfw), (Xam,Cd,YZfx),(Xam,Cd,YZfy),(Xam,Cd,YZfz),(Xam,Cd,YZga),(Xam,Cd,YZgb), (Xam,Cd,YZgc),(Xam,Cd,YZgd),(Xam,Cd,YZge),(Xam,Cd,YZgf),(Xam,Cd,YZgg),(Xam, Cd,YZgh),(Xam,Cd,YZgi),(Xam,Cd,YZgj),(Xam,Cd,YZgk),(Xam,Cd,YZgl),(Xam, Cd,YZgm),(Xam,Cd,YZgn),(Xam,Cd,YZgo),(Xam,Cd,YZgp),(Xam,Cd,YZgq),(Xam, Cd,YZgr),(Xam,Cd,YZgs),(Xam,Cd,YZgt),(Xam,Ce,YZa),(Xam,Ce,YZb),(Xam,Ce, YZc),(Xam,Ce,YZd),(Xam,Ce,YZe),(Xam,Ce,YZf),(Xam,Ce,YZg),(Xam,Ce,YZh),(Xam, Ce,YZi),(Xam,Ce,YZj),(Xam,Ce,YZk),(Xam,Ce,YZl),(Xam,Ce,YZm),(Xam,Ce,YZn), (Xam,Ce,YZo),(Xam,Ce,YZp),(Xam,Ce,YZq),(Xam,Ce,YZr),(Xam,Ce,YZs),(Xam, Ce,YZt),(Xam,Ce,YZu),(Xam,Ce,YZv),(Xam,Ce,YZw),(Xam,Ce,YZx),(Xam,Ce,YZy), (Xam,Ce,YZz),(Xam,Ce,YZaa),(Xam,Ce,YZab),(Xam,Ce,YZac),(Xam,Ce,YZad), (Xam,Ce,YZae),(Xam,Ce,YZaf),(Xam,Ce,YZag),(Xam,Ce,YZah),(Xam,Ce,YZai),(Xam, Ce,YZaj),(Xam,Ce,YZak),(Xam,Ce,YZal),(Xam,Ce,YZam),(Xam,Ce,YZan),(Xam, Ce,YZao),(Xam,Ce,YZap),(Xam,Ce,YZaq),(Xam,Ce,YZar),(Xam,Ce,YZas),(Xam,Ce, YZat),(Xam,Ce,YZau),(Xam,Ce,YZav),(Xam,Ce,YZaw),(Xam,Ce,YZax),(Xam,Ce,YZay), (Xam,Ce,YZaz),(Xam,Ce,YZba),(Xam,Ce,YZbb),(Xam,Ce,YZbc),(Xam,Ce,YZbd), (Xam,Ce,YZbe),(Xam,Ce,YZbf),(Xam,Ce,YZbg),(Xam,Ce,YZbh),(Xam,Ce,YZbi),(Xam, Ce,YZbj),(Xam,Ce,YZbk),(Xam,Ce,YZbl),(Xam,Ce,YZbm),(Xam,Ce,YZbn),(Xam, Ce,YZbo),(Xam,Ce,YZbp),(Xam,Ce,YZbq),(Xam,Ce,YZbr),(Xam,Ce,YZbs),(Xam, Ce,YZbt),(Xam,Ce,YZbu),(Xam,Ce,YZbv),(Xam,Ce,YZbw),(Xam,Ce,YZbx),(Xam,Ce, YZby),(Xam,Ce,YZbz),(Xam,Ce,YZca),(Xam,Ce,YZcb),(Xam,Ce,YZcc),(Xam,Ce, YZcd),(Xam,Ce,YZce),(Xam,Ce,YZcf),(Xam,Ce,YZcg),(Xam,Ce,YZch),(Xam,Ce,YZci), (Xam,Ce,YZcj),(Xam,Ce,YZck),(Xam,Ce,YZcl),(Xam,Ce,YZcm),(Xam,Ce,YZcn), (Xam,Ce,YZco),(Xam,Ce,YZcp),(Xam,Ce,YZcq),(Xam,Ce,YZcr),(Xam,Ce,YZcs),(Xam, Ce,YZct),(Xam,Ce,YZcu),(Xam,Ce,YZcv),(Xam,Ce,YZcw),(Xam,Ce,YZcx),(Xam, Ce,YZcy),(Xam,Ce,YZcz),(Xam,Ce,YZda),(Xam,Ce,YZdb),(Xam,Ce,YZdc),(Xam,Ce, YZdd),(Xam,Ce,YZde),(Xam,Ce,YZdf),(Xam,Ce,YZdg),(Xam,Ce,YZdh),(Xam,Ce,YZdi), (Xam,Ce,YZdj),(Xam,Ce,YZdk),(Xam,Ce,YZdl),(Xam,Ce,YZdm),(Xam,Ce,YZdn), (Xam,Ce,YZdo),(Xam,Ce,YZdp),(Xam,Ce,YZdq),(Xam,Ce,YZdr),(Xam,Ce,YZds), (Xam,Ce,Yzdt),(Xam,Ce,YZdu),(Xam,Ce,YZdv),(Xam,Ce,YZdw),(Xam,Ce,YZdx),(Xam, Ce,YZdy),(Xam,Ce,YZdz),(Xam,Ce,YZea),(Xam,Ce,YZeb),(Xam,Ce,YZec),(Xam, Ce,YZed),(Xam,Ce,YZee),(Xam,Ce,YZef),(Xam,Ce,YZeg),(Xam,Ce,YZeh),(Xam,Ce, YZei),(Xam,Ce,YZej),(Xam,Ce,YZek),(Xam,Ce,YZel),(Xam,Ce,YZem),(Xam,Ce,YZen), (Xam,Ce,YZeo),(Xam,Ce,YZep),(Xam,Ce,YZeq),(Xam,Ce,YZer),(Xam,Ce,YZes), (Xam,Ce,YZet),(Xam,Ce,YZeu),(Xam,Ce,YZev),(Xam,Ce,YZew),(Xam,Ce,YZex),(Xam, Ce,YZey),(Xam,Ce,YZez),(Xam,Ce,YZfa),(Xam,Ce,YZfb),(Xam,Ce,YZfc),(Xam, Ce,YZfd),(Xam,Ce,YZfe),(Xam,Ce,YZff),(Xam,Ce,YZfg),(Xam,Ce,YZfh),(Xam,Ce, YZfi),(Xam,Ce,YZfj),(Xam,Ce,YZfk),(Xam,Ce,YZfl),(Xam,Ce,YZfm),(Xam,Ce,YZfn), (Xam,Ce,YZfo),(Xam,Ce,YZfp),(Xam,Ce,YZfq),(Xam,Ce,YZfr),(Xam,Ce,YZfs),(Xam, Ce,YZft),(Xam,Ce,YZfu),(Xam,Ce,YZfv),(Xam,Ce,YZfw),(Xam,Ce,YZfx),(Xam,Ce, YZfy),(Xam,Ce,YZfz),(Xam,Ce,YZga),(Xam,Ce,YZgb),(Xam,Ce,YZgc),(Xam,Ce,YZgd), (Xam,Ce,YZge),(Xam,Ce,YZgf),(Xam,Ce,YZgg),(Xam,Ce,YZgh),(Xam,Ce,YZgi), (Xam,Ce,YZgj),(Xam,Ce,YZgk),(Xam,Ce,YZgl),(Xam,Ce,YZgm),(Xam,Ce,YZgn), (Xam,Ce,YZgo),(Xam,Ce,YZgp),(Xam,Ce,YZgq),(Xam,Ce,YZgr),(Xam,Ce,YZgs),(Xam, Ce,YZgt),(Xam,Cf,YZa),(Xam,Cf,YZb),(Xam,Cf,YZc),(Xam,Cf,YZd),(Xam,Cf,YZe), (Xam,Cf,YZf),(Xam,Cf,YZg),(Xam,Cf,YZh),(Xam,Cf,YZi),(Xam,Cf,YZj),(Xam, Cf,YZk),(Xam,Cf,YZl),(Xam,Cf,YZm),(Xam,Cf,YZn),(Xam,Cf,YZo),(Xam,Cf,YZp), (Xam,Cf,YZq),(Xam,Cf,YZr),(Xam,Cf,YZs),(Xam,Cf,YZt),(Xam,Cf,YZu),(Xam,Cf,YZv), (Xam,Cf,YZw),(Xam,Cf,YZx),(Xam,Cf,YZy),(Xam,Cf,YZz),(Xam,Cf,YZaa),(Xam, Cf,YZab),(Xam,Cf,YZac),(Xam,Cf,YZad),(Xam,Cf,YZae),(Xam,Cf,YZaf),(Xam,Cf, YZag),(Xam,Cf,YZah),(Xam,Cf,YZai),(Xam,Cf,YZaj),(Xam,Cf,YZak),(Xam,Cf,YZal), (Xam,Cf,YZam),(Xam,Cf,YZan),(Xam,Cf,YZao),(Xam,Cf,YZap),(Xam,Cf,YZaq),(Xam, Cf,YZar),(Xam,Cf,YZas),(Xam,Cf,YZat),(Xam,Cf,YZau),(Xam,Cf,YZav),(Xam, Cf,YZaw),(Xam,Cf,YZax),(Xam,Cf,YZay),(Xam,Cf,YZaz),(Xam,Cf,YZba),(Xam,Cf, YZbb),(Xam,Cf,YZbc),(Xam,Cf,YZbd),(Xam,Cf,YZbe),(Xam,Cf,YZbf),(Xam,Cf,YZbg), (Xam,Cf,YZbh),(Xam,Cf,YZbi),(Xam,Cf,YZbj),(Xam,Cf,YZbk),(Xam,Cf,YZbl),(Xam, Cf,YZbm),(Xam,Cf,YZbn),(Xam,Cf,YZbo),(Xam,Cf,YZbp),(Xam,Cf,YZbq),(Xam, Cf,YZbr),(Xam,Cf,YZbs),(Xam,Cf,YZbt),(Xam,Cf,YZbu),(Xam,Cf,YZbv),(Xam,Cf, YZbw),(Xam,Cf,YZbx),(Xam,Cf,YZby),(Xam,Cf,YZbz),(Xam,Cf,YZca),(Xam,Cf,YZcb), (Xam,Cf,YZcc),(Xam,Cf,YZcd),(Xam,Cf,YZce),(Xam,Cf,YZcf),(Xam,Cf,YZcg), (Xam,Cf,YZch),(Xam,Cf,YZci),(Xam,Cf,YZcj),(Xam,Cf,YZck),(Xam,Cf,YZcl),(Xam, Cf,YZcm),(Xam,Cf,YZcn),(Xam,Cf,YZco),(Xam,Cf,YZcp),(Xam,Cf,YZcq),(Xam, Cf,YZcr),(Xam,Cf,YZcs),(Xam,Cf,YZct),(Xam,Cf,YZcu),(Xam,Cf,YZcv),(Xam,Cf,YZcw), (Xam,Cf,YZcx),(Xam,Cf,YZcy),(Xam,Cf,YZcz),(Xam,Cf,YZda),(Xam,Cf,YZdb), (Xam,Cf,YZdc),(Xam,Cf,YZdd),(Xam,Cf,YZde),(Xam,Cf,YZdf),(Xam,Cf,YZdg),(Xam, Cf,YZdh),(Xam,Cf,YZdi),(Xam,Cf,YZdj),(Xam,Cf,YZdk),(Xam,Cf,YZdl),(Xam,Cf, YZdm),(Xam,Cf,YZdn),(Xam,Cf,YZdo),(Xam,Cf,YZdp),(Xam,Cf,YZdq),(Xam,Cf, YZdr),(Xam,Cf,YZds),(Xam,Cf,Yzdt),(Xam,Cf,YZdu),(Xam,Cf,YZdv),(Xam,Cf,YZdw), (Xam,Cf,YZdx),(Xam,Cf,YZdy),(Xam,Cf,YZdz),(Xam,Cf,YZea),(Xam,Cf,YZeb),(Xam, Cf,YZec),(Xam,Cf,YZed),(Xam,Cf,YZee),(Xam,Cf,YZef),(Xam,Cf,YZeg),(Xam, Cf,YZeh),(Xam,Cf,YZei),(Xam,Cf,YZej),(Xam,Cf,YZek),(Xam,Cf,YZel),(Xam,Cf,YZem), (Xam,Cf,YZen),(Xam,Cf,YZeo),(Xam,Cf,YZep),(Xam,Cf,YZeq),(Xam,Cf,YZer), (Xam,Cf,YZes),(Xam,Cf,YZet),(Xam,Cf,YZeu),(Xam,Cf,YZev),(Xam,Cf,YZew),(Xam, Cf,YZex),(Xam,Cf,YZey),(Xam,Cf,YZez),(Xam,Cf,YZfa),(Xam,Cf,YZfb),(Xam,Cf, YZfc),(Xam,Cf,YZfd),(Xam,Cf,YZfe),(Xam,Cf,YZff),(Xam,Cf,YZfg),(Xam,Cf,YZfh), (Xam,Cf,YZfi),(Xam,Cf,YZfj),(Xam,Cf,YZfk),(Xam,Cf,YZfl),(Xam,Cf,YZfm),(Xam, Cf,YZfn),(Xam,Cf,YZfo),(Xam,Cf,YZfp),(Xam,Cf,YZfq),(Xam,Cf,YZfr),(Xam,Cf, YZfs),(Xam,Cf,YZft),(Xam,Cf,YZfu),(Xam,Cf,YZfv),(Xam,Cf,YZfw),(Xam,Cf,YZfx), (Xam,Cf,YZfy),(Xam,Cf,YZfz),(Xam,Cf,YZga),(Xam,Cf,YZgb),(Xam,Cf,YZgc),(Xam, Cf,YZgd),(Xam,Cf,YZge),(Xam,Cf,YZgf),(Xam,Cf,YZgg),(Xam,Cf,YZgh),(Xam, Cf,YZgi),(Xam,Cf,YZgj),(Xam,Cf,YZgk),(Xam,Cf,YZgl),(Xam,Cf,YZgm),(Xam,Cf, YZgn),(Xam,Cf,YZgo),(Xam,Cf,YZgp),(Xam,Cf,YZgq),(Xam,Cf,YZgr),(Xam,Cf,YZgs), (Xam,Cf,YZgt), (Xan,Ca,YZa),(Xan,Ca,YZb),(Xan,Ca,YZc),(Xan,Ca,YZd),(Xan,Ca,YZe),(Xan,Ca,YZf), (Xan,Ca,YZg),(Xan,Ca,YZh),(Xan,Ca,YZi),(Xan,Ca,YZj),(Xan,Ca,YZk),(Xan,Ca,YZl), (Xan,Ca,YZm),(Xan,Ca,YZn),(Xan,Ca,YZo),(Xan,Ca,YZp),(Xan,Ca,YZq),(Xan,Ca, YZr),(Xan,Ca,YZs),(Xan,Ca,YZt),(Xan,Ca,YZu),(Xan,Ca,YZv),(Xan,Ca,YZw),(Xan, Ca,YZx),(Xan,Ca,YZy),(Xan,Ca,YZz),(Xan,Ca,YZaa),(Xan,Ca,YZab),(Xan,Ca,YZac), (Xan,Ca,YZad),(Xan,Ca,YZae),(Xan,Ca,YZaf),(Xan,Ca,YZag),(Xan,Ca,YZah),(Xan, Ca,YZai),(Xan,Ca,YZaj),(Xan,Ca,YZak),(Xan,Ca,YZal),(Xan,Ca,YZam),(Xan,Ca,YZan), (Xan,Ca,YZao),(Xan,Ca,YZap),(Xan,Ca,YZaq),(Xan,Ca,YZar),(Xan,Ca,YZas),(Xan, Ca,YZat),(Xan,Ca,YZau),(Xan,Ca,YZav),(Xan,Ca,YZaw),(Xan,Ca,YZax),(Xan,Ca, YZay),(Xan,Ca,YZaz),(Xan,Ca,YZba),(Xan,Ca,YZbb),(Xan,Ca,YZbc),(Xan,Ca,YZbd), (Xan,Ca,YZbe),(Xan,Ca,YZbf),(Xan,Ca,YZbg),(Xan,Ca,YZbh),(Xan,Ca,YZbi),(Xan, Ca,YZbj),(Xan,Ca,YZbk),(Xan,Ca,YZbl),(Xan,Ca,YZbm),(Xan,Ca,YZbn),(Xan,Ca,YZbo), (Xan,Ca,YZbp),(Xan,Ca,YZbq),(Xan,Ca,YZbr),(Xan,Ca,YZbs),(Xan,Ca,YZbt),(Xan, Ca,YZbu),(Xan,Ca,YZbv),(Xan,Ca,YZbw),(Xan,Ca,YZbx),(Xan,Ca,YZby),(Xan, Ca,YZbz),(Xan,Ca,YZca),(Xan,Ca,YZcb),(Xan,Ca,YZcc),(Xan,Ca,YZcd),(Xan,Ca,YZce), (Xan,Ca,YZcf),(Xan,Ca,YZcg),(Xan,Ca,YZch),(Xan,Ca,YZci),(Xan,Ca,YZcj),(Xan, Ca,YZck),(Xan,Ca,YZcl),(Xan,Ca,YZcm),(Xan,Ca,YZcn),(Xan,Ca,YZco),(Xan,Ca, YZcp),(Xan,Ca,YZcq),(Xan,Ca,YZcr),(Xan,Ca,YZcs),(Xan,Ca,YZct),(Xan,Ca,YZcu), (Xan,Ca,YZcv),(Xan,Ca,YZcw),(Xan,Ca,YZcx),(Xan,Ca,YZcy),(Xan,Ca,YZcz),(Xan, Ca,YZda),(Xan,Ca,YZdb),(Xan,Ca,YZdc),(Xan,Ca,YZdd),(Xan,Ca,YZde),(Xan,Ca,YZdf), (Xan,Ca,YZdg),(Xan,Ca,YZdh),(Xan,Ca,YZdi),(Xan,Ca,YZdj),(Xan,Ca,YZdk),(Xan, Ca,YZdl),(Xan,Ca,YZdm),(Xan,Ca,YZdn),(Xan,Ca,YZdo),(Xan,Ca,YZdp),(Xan, Ca,YZdq),(Xan,Ca,YZdr),(Xan,Ca,YZds),(Xan,Ca,Yzdt),(Xan,Ca,YZdu),(Xan,Ca,YZdv), (Xan,Ca,YZdw),(Xan,Ca,YZdx),(Xan,Ca,YZdy),(Xan,Ca,YZdz),(Xan,Ca,YZea),(Xan, Ca,YZeb),(Xan,Ca,YZec),(Xan,Ca,YZed),(Xan,Ca,YZee),(Xan,Ca,YZef),(Xan,Ca, YZeg),(Xan,Ca,YZeh),(Xan,Ca,YZei),(Xan,Ca,YZej),(Xan,Ca,YZek),(Xan,Ca,YZel), (Xan,Ca,YZem),(Xan,Ca,YZen),(Xan,Ca,YZeo),(Xan,Ca,YZep),(Xan,Ca,YZeq),(Xan, Ca,YZer),(Xan,Ca,YZes),(Xan,Ca,YZet),(Xan,Ca,YZeu),(Xan,Ca,YZev),(Xan,Ca,YZew), (Xan,Ca,YZex),(Xan,Ca,YZey),(Xan,Ca,YZez),(Xan,Ca,YZfa),(Xan,Ca,YZfb),(Xan, Ca,YZfc),(Xan,Ca,YZfd),(Xan,Ca,YZfe),(Xan,Ca,YZff),(Xan,Ca,YZfg),(Xan,Ca,YZfh), (Xan,Ca,YZfi),(Xan,Ca,YZfj),(Xan,Ca,YZfk),(Xan,Ca,YZfl),(Xan,Ca,YZfm),(Xan, Ca,YZfn),(Xan,Ca,YZfo),(Xan,Ca,YZfp),(Xan,Ca,YZfq),(Xan,Ca,YZfr),(Xan,Ca,YZfs), (Xan,Ca,YZft),(Xan,Ca,YZfu),(Xan,Ca,YZfv),(Xan,Ca,YZfw),(Xan,Ca,YZfx),(Xan, Ca,YZfy),(Xan,Ca,YZfz),(Xan,Ca,YZga),(Xan,Ca,YZgb),(Xan,Ca,YZgc),(Xan,Ca, YZgd),(Xan,Ca,YZge),(Xan,Ca,YZgf),(Xan,Ca,YZgg),(Xan,Ca,YZgh),(Xan,Ca,YZgi), (Xan,Ca,YZgj),(Xan,Ca,YZgk),(Xan,Ca,YZgl),(Xan,Ca,YZgm),(Xan,Ca,YZgn),(Xan, Ca,YZgo),(Xan,Ca,YZgp),(Xan,Ca,YZgq),(Xan,Ca,YZgr),(Xan,Ca,YZgs),(Xan,Ca,YZgt), (Xan,Cb,YZa),(Xan,Cb,YZb),(Xan,Cb,YZc),(Xan,Cb,YZd),(Xan,Cb,YZe),(Xan, Cb,YZf),(Xan,Cb,YZg),(Xan,Cb,YZh),(Xan,Cb,YZi),(Xan,Cb,YZj),(Xan,Cb,YZk),(Xan, Cb,YZl),(Xan,Cb,YZm),(Xan,Cb,YZn),(Xan,Cb,YZo),(Xan,Cb,YZp),(Xan,Cb,YZq), (Xan,Cb,YZr),(Xan,Cb,YZs),(Xan,Cb,YZt),(Xan,Cb,YZu),(Xan,Cb,YZv),(Xan,Cb,YZw), (Xan,Cb,YZx),(Xan,Cb,YZy),(Xan,Cb,YZz),(Xan,Cb,YZaa),(Xan,Cb,YZab),(Xan, Cb,YZac),(Xan,Cb,YZad),(Xan,Cb,YZae),(Xan,Cb,YZaf),(Xan,Cb,YZag),(Xan,Cb, YZah),(Xan,Cb,YZai),(Xan,Cb,YZaj),(Xan,Cb,YZak),(Xan,Cb,YZal),(Xan,Cb,YZam), (Xan,Cb,YZan),(Xan,Cb,YZao),(Xan,Cb,YZap),(Xan,Cb,YZaq),(Xan,Cb,YZar),(Xan, Cb,YZas),(Xan,Cb,YZat),(Xan,Cb,YZau),(Xan,Cb,YZav),(Xan,Cb,YZaw),(Xan,Cb,YZax), (Xan,Cb,YZay),(Xan,Cb,YZaz),(Xan,Cb,YZba),(Xan,Cb,YZbb),(Xan,Cb,YZbc), (Xan,Cb,YZbd),(Xan,Cb,YZbe),(Xan,Cb,YZbf),(Xan,Cb,YZbg),(Xan,Cb,YZbh),(Xan, Cb,YZbi),(Xan,Cb,YZbj),(Xan,Cb,YZbk),(Xan,Cb,YZbl),(Xan,Cb,YZbm),(Xan,Cb,YZbn), (Xan,Cb,YZbo),(Xan,Cb,YZbp),(Xan,Cb,YZbq),(Xan,Cb,YZbr),(Xan,Cb,YZbs), (Xan,Cb,YZbt),(Xan,Cb,YZbu),(Xan,Cb,YZbv),(Xan,Cb,YZbw),(Xan,Cb,YZbx),(Xan, Cb,YZby),(Xan,Cb,YZbz),(Xan,Cb,YZca),(Xan,Cb,YZcb),(Xan,Cb,YZcc),(Xan,Cb,YZcd), (Xan,Cb,YZce),(Xan,Cb,YZcf),(Xan,Cb,YZcg),(Xan,Cb,YZch),(Xan,Cb,YZci),(Xan, Cb,YZcj),(Xan,Cb,YZck),(Xan,Cb,YZcl),(Xan,Cb,YZcm),(Xan,Cb,YZcn),(Xan, Cb,YZco),(Xan,Cb,YZcp),(Xan,Cb,YZcq),(Xan,Cb,YZcr),(Xan,Cb,YZcs),(Xan,Cb,YZct), (Xan,Cb,YZcu),(Xan,Cb,YZcv),(Xan,Cb,YZcw),(Xan,Cb,YZcx),(Xan,Cb,YZcy), (Xan,Cb,YZcz),(Xan,Cb,YZda),(Xan,Cb,YZdb),(Xan,Cb,YZdc),(Xan,Cb,YZdd),(Xan, Cb,YZde),(Xan,Cb,YZdf),(Xan,Cb,YZdg),(Xan,Cb,YZdh),(Xan,Cb,YZdi),(Xan,Cb,YZdj), (Xan,Cb,YZdk),(Xan,Cb,YZdl),(Xan,Cb,YZdm),(Xan,Cb,YZdn),(Xan,Cb,YZdo), (Xan,Cb,YZdp),(Xan,Cb,YZdq),(Xan,Cb,YZdr),(Xan,Cb,YZds),(Xan,Cb,Yzdt),(Xan, Cb,YZdu),(Xan,Cb,YZdv),(Xan,Cb,YZdw),(Xan,Cb,YZdx),(Xan,Cb,YZdy),(Xan,Cb, YZdz),(Xan,Cb,YZea),(Xan,Cb,YZeb),(Xan,Cb,YZec),(Xan,Cb,YZed),(Xan,Cb,YZee), (Xan,Cb,YZef),(Xan,Cb,YZeg),(Xan,Cb,YZeh),(Xan,Cb,YZei),(Xan,Cb,YZej),(Xan, Cb,YZek),(Xan,Cb,YZel),(Xan,Cb,YZem),(Xan,Cb,YZen),(Xan,Cb,YZeo),(Xan,Cb,YZep), (Xan,Cb,YZeq),(Xan,Cb,YZer),(Xan,Cb,YZes),(Xan,Cb,YZet),(Xan,Cb,YZeu),(Xan, Cb,YZev),(Xan,Cb,YZew),(Xan,Cb,YZex),(Xan,Cb,YZey),(Xan,Cb,YZez),(Xan, Cb,YZfa),(Xan,Cb,YZfb),(Xan,Cb,YZfc),(Xan,Cb,YZfd),(Xan,Cb,YZfe),(Xan,Cb,YZff), (Xan,Cb,YZfg),(Xan,Cb,YZfh),(Xan,Cb,YZfi),(Xan,Cb,YZfj),(Xan,Cb,YZfk),(Xan, Cb,YZfl),(Xan,Cb,YZfm),(Xan,Cb,YZfn),(Xan,Cb,YZfo),(Xan,Cb,YZfp),(Xan,Cb,YZfq), (Xan,Cb,YZfr),(Xan,Cb,YZfs),(Xan,Cb,YZft),(Xan,Cb,YZfu),(Xan,Cb,YZfv),(Xan, Cb,YZfw),(Xan,Cb,YZfx),(Xan,Cb,YZfy),(Xan,Cb,YZfz),(Xan,Cb,YZga),(Xan,Cb,YZgb), (Xan,Cb,YZgc),(Xan,Cb,YZgd),(Xan,Cb,YZge),(Xan,Cb,YZgf),(Xan,Cb,YZgg), (Xan,Cb,YZgh),(Xan,Cb,YZgi),(Xan,Cb,YZgj),(Xan,Cb,YZgk),(Xan,Cb,YZgl),(Xan, Cb,YZgm),(Xan,Cb,YZgn),(Xan,Cb,YZgo),(Xan,Cb,YZgp),(Xan,Cb,YZgq),(Xan,Cb, YZgr),(Xan,Cb,YZgs),(Xan,Cb,YZgt),(Xan,Cc,YZa),(Xan,Cc,YZb),(Xan,Cc,YZc),(Xan, Cc,YZd),(Xan,Cc,YZe),(Xan,Cc,YZf),(Xan,Cc,YZg),(Xan,Cc,YZh),(Xan,Cc,YZi),(Xan, Cc,YZj),(Xan,Cc,YZk),(Xan,Cc,YZl),(Xan,Cc,YZm),(Xan,Cc,YZn),(Xan,Cc,YZo), (Xan,Cc,YZp),(Xan,Cc,YZq),(Xan,Cc,YZr),(Xan,Cc,YZs),(Xan,Cc,YZt),(Xan,Cc, YZu),(Xan,Cc,YZv),(Xan,Cc,YZw),(Xan,Cc,YZx),(Xan,Cc,YZy),(Xan,Cc,YZz),(Xan, Cc,YZaa),(Xan,Cc,YZab),(Xan,Cc,YZac),(Xan,Cc,YZad),(Xan,Cc,YZae),(Xan,Cc,YZaf), (Xan,Cc,YZag),(Xan,Cc,YZah),(Xan,Cc,YZai),(Xan,Cc,YZaj),(Xan,Cc,YZak),(Xan, Cc,YZal),(Xan,Cc,YZam),(Xan,Cc,YZan),(Xan,Cc,YZao),(Xan,Cc,YZap),(Xan,Cc, YZaq),(Xan,Cc,YZar),(Xan,Cc,YZas),(Xan,Cc,YZat),(Xan,Cc,YZau),(Xan,Cc,YZav), (Xan,Cc,YZaw),(Xan,Cc,YZax),(Xan,Cc,YZay),(Xan,Cc,YZaz),(Xan,Cc,YZba),(Xan, Cc,YZbb),(Xan,Cc,YZbc),(Xan,Cc,YZbd),(Xan,Cc,YZbe),(Xan,Cc,YZbf),(Xan,Cc,YZbg), (Xan,Cc,YZbh),(Xan,Cc,YZbi),(Xan,Cc,YZbj),(Xan,Cc,YZbk),(Xan,Cc,YZbl),(Xan, Cc,YZbm),(Xan,Cc,YZbn),(Xan,Cc,YZbo),(Xan,Cc,YZbp),(Xan,Cc,YZbq),(Xan, Cc,YZbr),(Xan,Cc,YZbs),(Xan,Cc,YZbt),(Xan,Cc,YZbu),(Xan,Cc,YZbv),(Xan,Cc,YZbw), (Xan,Cc,YZbx),(Xan,Cc,YZby),(Xan,Cc,YZbz),(Xan,Cc,YZca),(Xan,Cc,YZcb),(Xan, Cc,YZcc),(Xan,Cc,YZcd),(Xan,Cc,YZce),(Xan,Cc,YZcf),(Xan,Cc,YZcg),(Xan,Cc, YZch),(Xan,Cc,YZci),(Xan,Cc,YZcj),(Xan,Cc,YZck),(Xan,Cc,YZcl),(Xan,Cc,YZcm), (Xan,Cc,YZcn),(Xan,Cc,YZco),(Xan,Cc,YZcp),(Xan,Cc,YZcq),(Xan,Cc,YZcr),(Xan, Cc,YZcs),(Xan,Cc,YZct),(Xan,Cc,YZcu),(Xan,Cc,YZcv),(Xan,Cc,YZcw),(Xan,Cc,YZcx), (Xan,Cc,YZcy),(Xan,Cc,YZcz),(Xan,Cc,YZda),(Xan,Cc,YZdb),(Xan,Cc,YZdc),(Xan, Cc,YZdd),(Xan,Cc,YZde),(Xan,Cc,YZdf),(Xan,Cc,YZdg),(Xan,Cc,YZdh),(Xan,Cc, YZdi),(Xan,Cc,YZdj),(Xan,Cc,YZdk),(Xan,Cc,YZdl),(Xan,Cc,YZdm),(Xan,Cc,YZdn), (Xan,Cc,YZdo),(Xan,Cc,YZdp),(Xan,Cc,YZdq),(Xan,Cc,YZdr),(Xan,Cc,YZds),(Xan, Cc,Yzdt),(Xan,Cc,YZdu),(Xan,Cc,YZdv),(Xan,Cc,YZdw),(Xan,Cc,YZdx),(Xan,Cc, YZdy),(Xan,Cc,YZdz),(Xan,Cc,YZea),(Xan,Cc,YZeb),(Xan,Cc,YZec),(Xan,Cc,YZed), (Xan,Cc,YZee),(Xan,Cc,YZef),(Xan,Cc,YZeg),(Xan,Cc,YZeh),(Xan,Cc,YZei),(Xan, Cc,YZej),(Xan,Cc,YZek),(Xan,Cc,YZel),(Xan,Cc,YZem),(Xan,Cc,YZen),(Xan,Cc,YZeo), (Xan,Cc,YZep),(Xan,Cc,YZeq),(Xan,Cc,YZer),(Xan,Cc,YZes),(Xan,Cc,YZet),(Xan, Cc,YZeu),(Xan,Cc,YZev),(Xan,Cc,YZew),(Xan,Cc,YZex),(Xan,Cc,YZey),(Xan,Cc, YZez),(Xan,Cc,YZfa),(Xan,Cc,YZfb),(Xan,Cc,YZfc),(Xan,Cc,YZfd),(Xan,Cc,YZfe),(Xan, Cc,YZff),(Xan,Cc,YZfg),(Xan,Cc,YZfh),(Xan,Cc,YZfi),(Xan,Cc,YZfj),(Xan,Cc, YZfk),(Xan,Cc,YZfl),(Xan,Cc,YZfm),(Xan,Cc,YZfn),(Xan,Cc,YZfo),(Xan,Cc,YZfp),(Xan, Cc,YZfq),(Xan,Cc,YZfr),(Xan,Cc,YZfs),(Xan,Cc,YZft),(Xan,Cc,YZfu),(Xan,Cc, YZfv),(Xan,Cc,YZfw),(Xan,Cc,YZfx),(Xan,Cc,YZfy),(Xan,Cc,YZfz),(Xan,Cc,YZga), (Xan,Cc,YZgb),(Xan,Cc,YZgc),(Xan,Cc,YZgd),(Xan,Cc,YZge),(Xan,Cc,YZgf),(Xan, Cc,YZgg),(Xan,Cc,YZgh),(Xan,Cc,YZgi),(Xan,Cc,YZgj),(Xan,Cc,YZgk),(Xan,Cc,YZgl), (Xan,Cc,YZgm),(Xan,Cc,YZgn),(Xan,Cc,YZgo),(Xan,Cc,YZgp),(Xan,Cc,YZgq),(Xan, Cc,YZgr),(Xan,Cc,YZgs),(Xan,Cc,YZgt),(Xan,Cd,YZa),(Xan,Cd,YZb),(Xan,Cd, YZc),(Xan,Cd,YZd),(Xan,Cd,YZe),(Xan,Cd,YZf),(Xan,Cd,YZg),(Xan,Cd,YZh),(Xan, Cd,YZi),(Xan,Cd,YZj),(Xan,Cd,YZk),(Xan,Cd,YZl),(Xan,Cd,YZm),(Xan,Cd,YZn),(Xan, Cd,YZo),(Xan,Cd,YZp),(Xan,Cd,YZq),(Xan,Cd,YZr),(Xan,Cd,YZs),(Xan,Cd,YZt), (Xan,Cd,YZu),(Xan,Cd,YZv),(Xan,Cd,YZw),(Xan,Cd,YZx),(Xan,Cd,YZy),(Xan,Cd, YZz),(Xan,Cd,YZaa),(Xan,Cd,YZab),(Xan,Cd,YZac),(Xan,Cd,YZad),(Xan,Cd,YZae), (Xan,Cd,YZaf),(Xan,Cd,YZag),(Xan,Cd,YZah),(Xan,Cd,YZai),(Xan,Cd,YZaj),(Xan,Cd, YZak),(Xan,Cd,YZal),(Xan,Cd,YZam),(Xan,Cd,YZan),(Xan,Cd,YZao),(Xan,Cd,YZap), (Xan,Cd,YZaq),(Xan,Cd,YZar),(Xan,Cd,YZas),(Xan,Cd,YZat),(Xan,Cd,YZau),(Xan, Cd,YZav),(Xan,Cd,YZaw),(Xan,Cd,YZax),(Xan,Cd,YZay),(Xan,Cd,YZaz),(Xan,Cd, YZba),(Xan,Cd,YZbb),(Xan,Cd,YZbc),(Xan,Cd,YZbd),(Xan,Cd,YZbe),(Xan,Cd,YZbf), (Xan,Cd,YZbg),(Xan,Cd,YZbh),(Xan,Cd,YZbi),(Xan,Cd,YZbj),(Xan,Cd,YZbk),(Xan, Cd,YZbl),(Xan,Cd,YZbm),(Xan,Cd,YZbn),(Xan,Cd,YZbo),(Xan,Cd,YZbp),(Xan,Cd, YZbq),(Xan,Cd,YZbr),(Xan,Cd,YZbs),(Xan,Cd,YZbt),(Xan,Cd,YZbu),(Xan,Cd,YZbv), (Xan,Cd,YZbw),(Xan,Cd,YZbx),(Xan,Cd,YZby),(Xan,Cd,YZbz),(Xan,Cd,YZca),(Xan, Cd,YZcb),(Xan,Cd,YZcc),(Xan,Cd,YZcd),(Xan,Cd,YZce),(Xan,Cd,YZcf),(Xan,Cd, YZcg),(Xan,Cd,YZch),(Xan,Cd,YZci),(Xan,Cd,YZcj),(Xan,Cd,YZck),(Xan,Cd,YZcl), (Xan,Cd,YZcm),(Xan,Cd,YZcn),(Xan,Cd,YZco),(Xan,Cd,YZcp),(Xan,Cd,YZcq),(Xan, Cd,YZcr),(Xan,Cd,YZcs),(Xan,Cd,YZct),(Xan,Cd,YZcu),(Xan,Cd,YZcv),(Xan,Cd, YZcw),(Xan,Cd,YZcx),(Xan,Cd,YZcy),(Xan,Cd,YZcz),(Xan,Cd,YZda),(Xan,Cd,YZdb), (Xan,Cd,YZdc),(Xan,Cd,YZdd),(Xan,Cd,YZde),(Xan,Cd,YZdf),(Xan,Cd,YZdg),(Xan, Cd,YZdh),(Xan,Cd,YZdi),(Xan,Cd,YZdj),(Xan,Cd,YZdk),(Xan,Cd,YZdl),(Xan,Cd, YZdm),(Xan,Cd,YZdn),(Xan,Cd,YZdo),(Xan,Cd,YZdp),(Xan,Cd,YZdq),(Xan,Cd,YZdr), (Xan,Cd,YZds),(Xan,Cd,Yzdt),(Xan,Cd,YZdu),(Xan,Cd,YZdv),(Xan,Cd,YZdw),(Xan, Cd,YZdx),(Xan,Cd,YZdy),(Xan,Cd,YZdz),(Xan,Cd,YZea),(Xan,Cd,YZeb),(Xan, Cd,YZec),(Xan,Cd,YZed),(Xan,Cd,YZee),(Xan,Cd,YZef),(Xan,Cd,YZeg),(Xan,Cd,YZeh), (Xan,Cd,YZei),(Xan,Cd,YZej),(Xan,Cd,YZek),(Xan,Cd,YZel),(Xan,Cd,YZem),(Xan, Cd,YZen),(Xan,Cd,YZeo),(Xan,Cd,YZep),(Xan,Cd,YZeq),(Xan,Cd,YZer),(Xan, Cd,YZes),(Xan,Cd,YZet),(Xan,Cd,YZeu),(Xan,Cd,YZev),(Xan,Cd,YZew),(Xan,Cd,YZex), (Xan,Cd,YZey),(Xan,Cd,YZez),(Xan,Cd,YZfa),(Xan,Cd,YZfb),(Xan,Cd,YZfc),(Xan, Cd,YZfd),(Xan,Cd,YZfe),(Xan,Cd,YZff),(Xan,Cd,YZfg),(Xan,Cd,YZfh),(Xan,Cd, YZfi),(Xan,Cd,YZfj),(Xan,Cd,YZfk),(Xan,Cd,YZfl),(Xan,Cd,YZfm),(Xan,Cd,YZfn), (Xan,Cd,YZfo),(Xan,Cd,YZfp),(Xan,Cd,YZfq),(Xan,Cd,YZfr),(Xan,Cd,YZfs),(Xan,Cd, YZft),(Xan,Cd,YZfu),(Xan,Cd,YZfv),(Xan,Cd,YZfw),(Xan,Cd,YZfx),(Xan,Cd,YZfy), (Xan,Cd,YZfz),(Xan,Cd,YZga),(Xan,Cd,YZgb),(Xan,Cd,YZgc),(Xan,Cd,YZgd),(Xan, Cd,YZge),(Xan,Cd,YZgf),(Xan,Cd,YZgg),(Xan,Cd,YZgh),(Xan,Cd,YZgi),(Xan,Cd, YZgj),(Xan,Cd,YZgk),(Xan,Cd,YZgl),(Xan,Cd,YZgm),(Xan,Cd,YZgn),(Xan,Cd,YZgo), (Xan,Cd,YZgp),(Xan,Cd,YZgq),(Xan,Cd,YZgr),(Xan,Cd,YZgs),(Xan,Cd,YZgt),(Xan, Ce,YZa),(Xan,Ce,YZb),(Xan,Ce,YZc),(Xan,Ce,YZd),(Xan,Ce,YZe),(Xan,Ce,YZf),(Xan, Ce,YZg),(Xan,Ce,YZh),(Xan,Ce,YZi),(Xan,Ce,YZj),(Xan,Ce,YZk),(Xan,Ce,YZl), (Xan,Ce,YZm),(Xan,Ce,YZn),(Xan,Ce,YZo),(Xan,Ce,YZp),(Xan,Ce,YZq),(Xan,Ce,YZr), (Xan,Ce,YZs),(Xan,Ce,YZt),(Xan,Ce,YZu),(Xan,Ce,YZv),(Xan,Ce,YZw),(Xan,Ce, YZx),(Xan,Ce,YZy),(Xan,Ce,YZz),(Xan,Ce,YZaa),(Xan,Ce,YZab),(Xan,Ce,YZac),(Xan, Ce,YZad),(Xan,Ce,YZae),(Xan,Ce,YZaf),(Xan,Ce,YZag),(Xan,Ce,YZah),(Xan,Ce, YZai),(Xan,Ce,YZaj),(Xan,Ce,YZak),(Xan,Ce,YZal),(Xan,Ce,YZam),(Xan,Ce,YZan), (Xan,Ce,YZao),(Xan,Ce,YZap),(Xan,Ce,YZaq),(Xan,Ce,YZar),(Xan,Ce,YZas),(Xan, Ce,YZat),(Xan,Ce,YZau),(Xan,Ce,YZav),(Xan,Ce,YZaw),(Xan,Ce,YZax),(Xan,Ce,YZay), (Xan,Ce,YZaz),(Xan,Ce,YZba),(Xan,Ce,YZbb),(Xan,Ce,YZbc),(Xan,Ce,YZbd),(Xan, Ce,YZbe),(Xan,Ce,YZbf),(Xan,Ce,YZbg),(Xan,Ce,YZbh),(Xan,Ce,YZbi),(Xan,Ce, YZbj),(Xan,Ce,YZbk),(Xan,Ce,YZbl),(Xan,Ce,YZbm),(Xan,Ce,YZbn),(Xan,Ce,YZbo), (Xan,Ce,YZbp),(Xan,Ce,YZbq),(Xan,Ce,YZbr),(Xan,Ce,YZbs),(Xan,Ce,YZbt),(Xan, Ce,YZbu),(Xan,Ce,YZbv),(Xan,Ce,YZbw),(Xan,Ce,YZbx),(Xan,Ce,YZby),(Xan,Ce, YZbz),(Xan,Ce,YZca),(Xan,Ce,YZcb),(Xan,Ce,YZcc),(Xan,Ce,YZcd),(Xan,Ce,YZce), (Xan,Ce,YZcf),(Xan,Ce,YZcg),(Xan,Ce,YZch),(Xan,Ce,YZci),(Xan,Ce,YZcj),(Xan,Ce, YZck),(Xan,Ce,YZcl),(Xan,Ce,YZcm),(Xan,Ce,YZcn),(Xan,Ce,YZco),(Xan,Ce,YZcp), (Xan,Ce,YZcq),(Xan,Ce,YZcr),(Xan,Ce,YZcs),(Xan,Ce,YZct),(Xan,Ce,YZcu),(Xan, Ce,YZcv),(Xan,Ce,YZcw),(Xan,Ce,YZcx),(Xan,Ce,YZcy),(Xan,Ce,YZcz),(Xan,Ce,YZda), (Xan,Ce,YZdb),(Xan,Ce,YZdc),(Xan,Ce,YZdd),(Xan,Ce,YZde),(Xan,Ce,YZdf),(Xan, Ce,YZdg),(Xan,Ce,YZdh),(Xan,Ce,YZdi),(Xan,Ce,YZdj),(Xan,Ce,YZdk),(Xan,Ce, YZdl),(Xan,Ce,YZdm),(Xan,Ce,YZdn),(Xan,Ce,YZdo),(Xan,Ce,YZdp),(Xan,Ce,YZdq), (Xan,Ce,YZdr),(Xan,Ce,YZds),(Xan,Ce,Yzdt),(Xan,Ce,YZdu),(Xan,Ce,YZdv),(Xan, Ce,YZdw),(Xan,Ce,YZdx),(Xan,Ce,YZdy),(Xan,Ce,YZdz),(Xan,Ce,YZea),(Xan,Ce, YZeb),(Xan,Ce,YZec),(Xan,Ce,YZed),(Xan,Ce,YZee),(Xan,Ce,YZef),(Xan,Ce,YZeg), (Xan,Ce,YZeh),(Xan,Ce,YZei),(Xan,Ce,YZej),(Xan,Ce,YZek),(Xan,Ce,YZel),(Xan,Ce, YZem),(Xan,Ce,YZen),(Xan,Ce,YZeo),(Xan,Ce,YZep),(Xan,Ce,YZeq),(Xan,Ce,YZer), (Xan,Ce,YZes),(Xan,Ce,YZet),(Xan,Ce,YZeu),(Xan,Ce,YZev),(Xan,Ce,YZew),(Xan, Ce,YZex),(Xan,Ce,YZey),(Xan,Ce,YZez),(Xan,Ce,YZfa),(Xan,Ce,YZfb),(Xan,Ce, YZfc),(Xan,Ce,YZfd),(Xan,Ce,YZfe),(Xan,Ce,YZff),(Xan,Ce,YZfg),(Xan,Ce,YZfh),(Xan, Ce,YZfi),(Xan,Ce,YZfj),(Xan,Ce,YZfk),(Xan,Ce,YZfl),(Xan,Ce,YZfm),(Xan,Ce, YZfn),(Xan,Ce,YZfo),(Xan,Ce,YZfp),(Xan,Ce,YZfq),(Xan,Ce,YZfr),(Xan,Ce,YZfs),(Xan, Ce,YZft),(Xan,Ce,YZfu),(Xan,Ce,YZfv),(Xan,Ce,YZfw),(Xan,Ce,YZfx),(Xan,Ce, YZfy),(Xan,Ce,YZfz),(Xan,Ce,YZga),(Xan,Ce,YZgb),(Xan,Ce,YZgc),(Xan,Ce,YZgd), (Xan,Ce,YZge),(Xan,Ce,YZgf),(Xan,Ce,YZgg),(Xan,Ce,YZgh),(Xan,Ce,YZgi),(Xan, Ce,YZgj),(Xan,Ce,YZgk),(Xan,Ce,YZgl),(Xan,Ce,YZgm),(Xan,Ce,YZgn),(Xan,Ce,YZgo), (Xan,Ce,YZgp),(Xan,Ce,YZgq),(Xan,Ce,YZgr),(Xan,Ce,YZgs),(Xan,Ce,YZgt),(Xan, Cf,YZa),(Xan,Cf,YZb),(Xan,Cf,YZc),(Xan,Cf,YZd),(Xan,Cf,YZe),(Xan,Cf,YZf), (Xan,Cf,YZg),(Xan,Cf,YZh),(Xan,Cf,YZi),(Xan,Cf,YZj),(Xan,Cf,YZk),(Xan,Cf,YZl), (Xan,Cf,YZm),(Xan,Cf,YZn),(Xan,Cf,YZo),(Xan,Cf,YZp),(Xan,Cf,YZq),(Xan,Cf,YZr), (Xan,Cf,YZs),(Xan,Cf,YZt),(Xan,Cf,YZu),(Xan,Cf,YZv),(Xan,Cf,YZw),(Xan,Cf,YZx), (Xan,Cf,YZy),(Xan,Cf,YZz),(Xan,Cf,YZaa),(Xan,Cf,YZab),(Xan,Cf,YZac),(Xan, Cf,YZad),(Xan,Cf,YZae),(Xan,Cf,YZaf),(Xan,Cf,YZag),(Xan,Cf,YZah),(Xan,Cf,YZai), (Xan,Cf,YZaj),(Xan,Cf,YZak),(Xan,Cf,YZal),(Xan,Cf,YZam),(Xan,Cf,YZan),(Xan, Cf,YZao),(Xan,Cf,YZap),(Xan,Cf,YZaq),(Xan,Cf,YZar),(Xan,Cf,YZas),(Xan,Cf,YZat), (Xan,Cf,YZau),(Xan,Cf,YZav),(Xan,Cf,YZaw),(Xan,Cf,YZax),(Xan,Cf,YZay),(Xan, Cf,YZaz),(Xan,Cf,YZba),(Xan,Cf,YZbb),(Xan,Cf,YZbc),(Xan,Cf,YZbd),(Xan,Cf,YZbe), (Xan,Cf,YZbf),(Xan,Cf,YZbg),(Xan,Cf,YZbh),(Xan,Cf,YZbi),(Xan,Cf,YZbj),(Xan, Cf,YZbk),(Xan,Cf,YZbl),(Xan,Cf,YZbm),(Xan,Cf,YZbn),(Xan,Cf,YZbo),(Xan,Cf,YZbp), (Xan,Cf,YZbq),(Xan,Cf,YZbr),(Xan,Cf,YZbs),(Xan,Cf,YZbt),(Xan,Cf,YZbu),(Xan, Cf,YZbv),(Xan,Cf,YZbw),(Xan,Cf,YZbx),(Xan,Cf,YZby),(Xan,Cf,YZbz),(Xan,Cf,YZca), (Xan,Cf,YZcb),(Xan,Cf,YZcc),(Xan,Cf,YZcd),(Xan,Cf,YZce),(Xan,Cf,YZcf),(Xan, Cf,YZcg),(Xan,Cf,YZch),(Xan,Cf,YZci),(Xan,Cf,YZcj),(Xan,Cf,YZck),(Xan,Cf,YZcl), (Xan,Cf,YZcm),(Xan,Cf,YZcn),(Xan,Cf,YZco),(Xan,Cf,YZcp),(Xan,Cf,YZcq),(Xan, Cf,YZcr),(Xan,Cf,YZcs),(Xan,Cf,YZct),(Xan,Cf,YZcu),(Xan,Cf,YZcv),(Xan,Cf, YZcw),(Xan,Cf,YZcx),(Xan,Cf,YZcy),(Xan,Cf,YZcz),(Xan,Cf,YZda),(Xan,Cf,YZdb), (Xan,Cf,YZdc),(Xan,Cf,YZdd),(Xan,Cf,YZde),(Xan,Cf,YZdf),(Xan,Cf,YZdg),(Xan,Cf, YZdh),(Xan,Cf,YZdi),(Xan,Cf,YZdj),(Xan,Cf,YZdk),(Xan,Cf,YZdl),(Xan,Cf,YZdm), (Xan,Cf,YZdn),(Xan,Cf,YZdo),(Xan,Cf,YZdp),(Xan,Cf,YZdq),(Xan,Cf,YZdr),(Xan, Cf,YZds),(Xan,Cf,Yzdt),(Xan,Cf,YZdu),(Xan,Cf,YZdv),(Xan,Cf,YZdw),(Xan,Cf,YZdx), (Xan,Cf,YZdy),(Xan,Cf,YZdz),(Xan,Cf,YZea),(Xan,Cf,YZeb),(Xan,Cf,YZec),(Xan, Cf,YZed),(Xan,Cf,YZee),(Xan,Cf,YZef),(Xan,Cf,YZeg),(Xan,Cf,YZeh),(Xan,Cf,YZei), (Xan,Cf,YZej),(Xan,Cf,YZek),(Xan,Cf,YZel),(Xan,Cf,YZem),(Xan,Cf,YZen),(Xan, Cf,YZeo),(Xan,Cf,YZep),(Xan,Cf,YZeq),(Xan,Cf,YZer),(Xan,Cf,YZes),(Xan,Cf,YZet), (Xan,Cf,YZeu),(Xan,Cf,YZev),(Xan,Cf,YZew),(Xan,Cf,YZex),(Xan,Cf,YZey),(Xan, Cf,YZez),(Xan,Cf,YZfa),(Xan,Cf,YZfb),(Xan,Cf,YZfc),(Xan,Cf,YZfd),(Xan,Cf,YZfe), (Xan,Cf,YZff),(Xan,Cf,YZfg),(Xan,Cf,YZfh),(Xan,Cf,YZfi),(Xan,Cf,YZfj),(Xan,Cf, YZfk),(Xan,Cf,YZfl),(Xan,Cf,YZfm),(Xan,Cf,YZfn),(Xan,Cf,YZfo),(Xan,Cf,YZfp),(Xan, Cf,YZfq),(Xan,Cf,YZfr),(Xan,Cf,YZfs),(Xan,Cf,YZft),(Xan,Cf,YZfu),(Xan,Cf,YZfv), (Xan,Cf,YZfw),(Xan,Cf,YZfx),(Xan,Cf,YZfy),(Xan,Cf,YZfz),(Xan,Cf,YZga),(Xan, Cf,YZgb),(Xan,Cf,YZgc),(Xan,Cf,YZgd),(Xan,Cf,YZge),(Xan,Cf,YZgf),(Xan,Cf,YZgg), (Xan,Cf,YZgh),(Xan,Cf,YZgi),(Xan,Cf,YZgj),(Xan,Cf,YZgk),(Xan,Cf,YZgl),(Xan, Cf,YZgm),(Xan,Cf,YZgn),(Xan,Cf,YZgo),(Xan,Cf,YZgp),(Xan,Cf,YZgq),(Xan,Cf, YZgr),(Xan,Cf,YZgs),(Xan,Cf,YZgt), (Xao,Ca,YZa),(Xao,Ca,YZb),(Xao,Ca,YZc),(Xao,Ca,YZd),(Xao,Ca,YZe),(Xao,Ca,YZf), (Xao,Ca,YZg),(Xao,Ca,YZh),(Xao,Ca,YZi),(Xao,Ca,YZj),(Xao,Ca,YZk),(Xao,Ca,YZl), (Xao,Ca,YZm),(Xao,Ca,YZn),(Xao,Ca,YZo),(Xao,Ca,YZp),(Xao,Ca,YZq),(Xao,Ca, YZr),(Xao,Ca,YZs),(Xao,Ca,YZt),(Xao,Ca,YZu),(Xao,Ca,YZv),(Xao,Ca,YZw),(Xao, Ca,YZx),(Xao,Ca,YZy),(Xao,Ca,YZz),(Xao,Ca,YZaa),(Xao,Ca,YZab),(Xao,Ca,YZac), (Xao,Ca,YZad),(Xao,Ca,YZae),(Xao,Ca,YZaf),(Xao,Ca,YZag),(Xao,Ca,YZah),(Xao, Ca,YZai),(Xao,Ca,YZaj),(Xao,Ca,YZak),(Xao,Ca,YZal),(Xao,Ca,YZam),(Xao,Ca,YZan), (Xao,Ca,YZao),(Xao,Ca,YZap),(Xao,Ca,YZaq),(Xao,Ca,YZar),(Xao,Ca,YZas),(Xao, Ca,YZat),(Xao,Ca,YZau),(Xao,Ca,YZav),(Xao,Ca,YZaw),(Xao,Ca,YZax),(Xao,Ca, YZay),(Xao,Ca,YZaz),(Xao,Ca,YZba),(Xao,Ca,YZbb),(Xao,Ca,YZbc),(Xao,Ca,YZbd), (Xao,Ca,YZbe),(Xao,Ca,YZbf),(Xao,Ca,YZbg),(Xao,Ca,YZbh),(Xao,Ca,YZbi),(Xao, Ca,YZbj),(Xao,Ca,YZbk),(Xao,Ca,YZbl),(Xao,Ca,YZbm),(Xao,Ca,YZbn),(Xao,Ca,YZbo), (Xao,Ca,YZbp),(Xao,Ca,YZbq),(Xao,Ca,YZbr),(Xao,Ca,YZbs),(Xao,Ca,YZbt),(Xao, Ca,YZbu),(Xao,Ca,YZbv),(Xao,Ca,YZbw),(Xao,Ca,YZbx),(Xao,Ca,YZby),(Xao, Ca,YZbz),(Xao,Ca,YZca),(Xao,Ca,YZcb),(Xao,Ca,YZcc),(Xao,Ca,YZcd),(Xao,Ca,YZce), (Xao,Ca,YZcf),(Xao,Ca,YZcg),(Xao,Ca,YZch),(Xao,Ca,YZci),(Xao,Ca,YZcj),(Xao, Ca,YZck),(Xao,Ca,YZcl),(Xao,Ca,YZcm),(Xao,Ca,YZcn),(Xao,Ca,YZco),(Xao,Ca, YZcp),(Xao,Ca,YZcq),(Xao,Ca,YZcr),(Xao,Ca,YZcs),(Xao,Ca,YZct),(Xao,Ca,YZcu), (Xao,Ca,YZcv),(Xao,Ca,YZcw),(Xao,Ca,YZcx),(Xao,Ca,YZcy),(Xao,Ca,YZcz),(Xao, Ca,YZda),(Xao,Ca,YZdb),(Xao,Ca,YZdc),(Xao,Ca,YZdd),(Xao,Ca,YZde),(Xao,Ca,YZdf), (Xao,Ca,YZdg),(Xao,Ca,YZdh),(Xao,Ca,YZdi),(Xao,Ca,YZdj),(Xao,Ca,YZdk),(Xao, Ca,YZdl),(Xao,Ca,YZdm),(Xao,Ca,YZdn),(Xao,Ca,YZdo),(Xao,Ca,YZdp),(Xao, Ca,YZdq),(Xao,Ca,YZdr),(Xao,Ca,YZds),(Xao,Ca,Yzdt),(Xao,Ca,YZdu),(Xao,Ca,YZdv), (Xao,Ca,YZdw),(Xao,Ca,YZdx),(Xao,Ca,YZdy),(Xao,Ca,YZdz),(Xao,Ca,YZea),(Xao, Ca,YZeb),(Xao,Ca,YZec),(Xao,Ca,YZed),(Xao,Ca,YZee),(Xao,Ca,YZef),(Xao,Ca, YZeg),(Xao,Ca,YZeh),(Xao,Ca,YZei),(Xao,Ca,YZej),(Xao,Ca,YZek),(Xao,Ca,YZel), (Xao,Ca,YZem),(Xao,Ca,YZen),(Xao,Ca,YZeo),(Xao,Ca,YZep),(Xao,Ca,YZeq),(Xao, Ca,YZer),(Xao,Ca,YZes),(Xao,Ca,YZet),(Xao,Ca,YZeu),(Xao,Ca,YZev),(Xao,Ca,YZew), (Xao,Ca,YZex),(Xao,Ca,YZey),(Xao,Ca,YZez),(Xao,Ca,YZfa),(Xao,Ca,YZfb),(Xao, Ca,YZfc),(Xao,Ca,YZfd),(Xao,Ca,YZfe),(Xao,Ca,YZff),(Xao,Ca,YZfg),(Xao,Ca,YZfh), (Xao,Ca,YZfi),(Xao,Ca,YZfj),(Xao,Ca,YZfk),(Xao,Ca,YZfl),(Xao,Ca,YZfm),(Xao, Ca,YZfn),(Xao,Ca,YZfo),(Xao,Ca,YZfp),(Xao,Ca,YZfq),(Xao,Ca,YZfr),(Xao,Ca,YZfs), (Xao,Ca,YZft),(Xao,Ca,YZfu),(Xao,Ca,YZfv),(Xao,Ca,YZfw),(Xao,Ca,YZfx),(Xao, Ca,YZfy),(Xao,Ca,YZfz),(Xao,Ca,YZga),(Xao,Ca,YZgb),(Xao,Ca,YZgc),(Xao,Ca, YZgd),(Xao,Ca,YZge),(Xao,Ca,YZgf),(Xao,Ca,YZgg),(Xao,Ca,YZgh),(Xao,Ca,YZgi), (Xao,Ca,YZgj),(Xao,Ca,YZgk),(Xao,Ca,YZgl),(Xao,Ca,YZgm),(Xao,Ca,YZgn),(Xao, Ca,YZgo),(Xao,Ca,YZgp),(Xao,Ca,YZgq),(Xao,Ca,YZgr),(Xao,Ca,YZgs),(Xao,Ca,YZgt), (Xao,Cb,YZa),(Xao,Cb,YZb),(Xao,Cb,YZc),(Xao,Cb,YZd),(Xao,Cb,YZe),(Xao, Cb,YZf),(Xao,Cb,YZg),(Xao,Cb,YZh),(Xao,Cb,YZi),(Xao,Cb,YZj),(Xao,Cb,YZk),(Xao, Cb,YZl),(Xao,Cb,YZm),(Xao,Cb,YZn),(Xao,Cb,YZo),(Xao,Cb,YZp),(Xao,Cb,YZq), (Xao,Cb,YZr),(Xao,Cb,YZs),(Xao,Cb,YZt),(Xao,Cb,YZu),(Xao,Cb,YZv),(Xao,Cb,YZw), (Xao,Cb,YZx),(Xao,Cb,YZy),(Xao,Cb,YZz),(Xao,Cb,YZaa),(Xao,Cb,YZab),(Xao, Cb,YZac),(Xao,Cb,YZad),(Xao,Cb,YZae),(Xao,Cb,YZaf),(Xao,Cb,YZag),(Xao,Cb, YZah),(Xao,Cb,YZai),(Xao,Cb,YZaj),(Xao,Cb,YZak),(Xao,Cb,YZal),(Xao,Cb,YZam), (Xao,Cb,YZan),(Xao,Cb,YZao),(Xao,Cb,YZap),(Xao,Cb,YZaq),(Xao,Cb,YZar),(Xao, Cb,YZas),(Xao,Cb,YZat),(Xao,Cb,YZau),(Xao,Cb,YZav),(Xao,Cb,YZaw),(Xao,Cb,YZax), (Xao,Cb,YZay),(Xao,Cb,YZaz),(Xao,Cb,YZba),(Xao,Cb,YZbb),(Xao,Cb,YZbc), (Xao,Cb,YZbd),(Xao,Cb,YZbe),(Xao,Cb,YZbf),(Xao,Cb,YZbg),(Xao,Cb,YZbh),(Xao, Cb,YZbi),(Xao,Cb,YZbj),(Xao,Cb,YZbk),(Xao,Cb,YZbl),(Xao,Cb,YZbm),(Xao,Cb,YZbn), (Xao,Cb,YZbo),(Xao,Cb,YZbp),(Xao,Cb,YZbq),(Xao,Cb,YZbr),(Xao,Cb,YZbs), (Xao,Cb,YZbt),(Xao,Cb,YZbu),(Xao,Cb,YZbv),(Xao,Cb,YZbw),(Xao,Cb,YZbx),(Xao, Cb,YZby),(Xao,Cb,YZbz),(Xao,Cb,YZca),(Xao,Cb,YZcb),(Xao,Cb,YZcc),(Xao,Cb,YZcd), (Xao,Cb,YZce),(Xao,Cb,YZcf),(Xao,Cb,YZcg),(Xao,Cb,YZch),(Xao,Cb,YZci),(Xao, Cb,YZcj),(Xao,Cb,YZck),(Xao,Cb,YZcl),(Xao,Cb,YZcm),(Xao,Cb,YZcn),(Xao, Cb,YZco),(Xao,Cb,YZcp),(Xao,Cb,YZcq),(Xao,Cb,YZcr),(Xao,Cb,YZcs),(Xao,Cb,YZct), (Xao,Cb,YZcu),(Xao,Cb,YZcv),(Xao,Cb,YZcw),(Xao,Cb,YZcx),(Xao,Cb,YZcy), (Xao,Cb,YZcz),(Xao,Cb,YZda),(Xao,Cb,YZdb),(Xao,Cb,YZdc),(Xao,Cb,YZdd),(Xao, Cb,YZde),(Xao,Cb,YZdf),(Xao,Cb,YZdg),(Xao,Cb,YZdh),(Xao,Cb,YZdi),(Xao,Cb,YZdj), (Xao,Cb,YZdk),(Xao,Cb,YZdl),(Xao,Cb,YZdm),(Xao,Cb,YZdn),(Xao,Cb,YZdo), (Xao,Cb,YZdp),(Xao,Cb,YZdq),(Xao,Cb,YZdr),(Xao,Cb,YZds),(Xao,Cb,Yzdt),(Xao, Cb,YZdu),(Xao,Cb,YZdv),(Xao,Cb,YZdw),(Xao,Cb,YZdx),(Xao,Cb,YZdy),(Xao,Cb, YZdz),(Xao,Cb,YZea),(Xao,Cb,YZeb),(Xao,Cb,YZec),(Xao,Cb,YZed),(Xao,Cb,YZee), (Xao,Cb,YZef),(Xao,Cb,YZeg),(Xao,Cb,YZeh),(Xao,Cb,YZei),(Xao,Cb,YZej),(Xao, Cb,YZek),(Xao,Cb,YZel),(Xao,Cb,YZem),(Xao,Cb,YZen),(Xao,Cb,YZeo),(Xao,Cb,YZep), (Xao,Cb,YZeq),(Xao,Cb,YZer),(Xao,Cb,YZes),(Xao,Cb,YZet),(Xao,Cb,YZeu),(Xao, Cb,YZev),(Xao,Cb,YZew),(Xao,Cb,YZex),(Xao,Cb,YZey),(Xao,Cb,YZez),(Xao, Cb,YZfa),(Xao,Cb,YZfb),(Xao,Cb,YZfc),(Xao,Cb,YZfd),(Xao,Cb,YZfe),(Xao,Cb,YZff), (Xao,Cb,YZfg),(Xao,Cb,YZfh),(Xao,Cb,YZfi),(Xao,Cb,YZfj),(Xao,Cb,YZfk),(Xao, Cb,YZfl),(Xao,Cb,YZfm),(Xao,Cb,YZfn),(Xao,Cb,YZfo),(Xao,Cb,YZfp),(Xao,Cb,YZfq), (Xao,Cb,YZfr),(Xao,Cb,YZfs),(Xao,Cb,YZft),(Xao,Cb,YZfu),(Xao,Cb,YZfv),(Xao, Cb,YZfw),(Xao,Cb,YZfx),(Xao,Cb,YZfy),(Xao,Cb,YZfz),(Xao,Cb,YZga),(Xao,Cb,YZgb), (Xao,Cb,YZgc),(Xao,Cb,YZgd),(Xao,Cb,YZge),(Xao,Cb,YZgf),(Xao,Cb,YZgg), (Xao,Cb,YZgh),(Xao,Cb,YZgi),(Xao,Cb,YZgj),(Xao,Cb,YZgk),(Xao,Cb,YZgl),(Xao, Cb,YZgm),(Xao,Cb,YZgn),(Xao,Cb,YZgo),(Xao,Cb,YZgp),(Xao,Cb,YZgq),(Xao,Cb, YZgr),(Xao,Cb,YZgs),(Xao,Cb,YZgt),(Xao,Cc,YZa),(Xao,Cc,YZb),(Xao,Cc,YZc),(Xao, Cc,YZd),(Xao,Cc,YZe),(Xao,Cc,YZf),(Xao,Cc,YZg),(Xao,Cc,YZh),(Xao,Cc,YZi),(Xao, Cc,YZj),(Xao,Cc,YZk),(Xao,Cc,YZl),(Xao,Cc,YZm),(Xao,Cc,YZn),(Xao,Cc,YZo), (Xao,Cc,YZp),(Xao,Cc,YZq),(Xao,Cc,YZr),(Xao,Cc,YZs),(Xao,Cc,YZt),(Xao,Cc, YZu),(Xao,Cc,YZv),(Xao,Cc,YZw),(Xao,Cc,YZx),(Xao,Cc,YZy),(Xao,Cc,YZz),(Xao, Cc,YZaa),(Xao,Cc,YZab),(Xao,Cc,YZac),(Xao,Cc,YZad),(Xao,Cc,YZae),(Xao,Cc,YZaf), (Xao,Cc,YZag),(Xao,Cc,YZah),(Xao,Cc,YZai),(Xao,Cc,YZaj),(Xao,Cc,YZak),(Xao, Cc,YZal),(Xao,Cc,YZam),(Xao,Cc,YZan),(Xao,Cc,YZao),(Xao,Cc,YZap),(Xao,Cc, YZaq),(Xao,Cc,YZar),(Xao,Cc,YZas),(Xao,Cc,YZat),(Xao,Cc,YZau),(Xao,Cc,YZav), (Xao,Cc,YZaw),(Xao,Cc,YZax),(Xao,Cc,YZay),(Xao,Cc,YZaz),(Xao,Cc,YZba),(Xao, Cc,YZbb),(Xao,Cc,YZbc),(Xao,Cc,YZbd),(Xao,Cc,YZbe),(Xao,Cc,YZbf),(Xao,Cc,YZbg), (Xao,Cc,YZbh),(Xao,Cc,YZbi),(Xao,Cc,YZbj),(Xao,Cc,YZbk),(Xao,Cc,YZbl),(Xao, Cc,YZbm),(Xao,Cc,YZbn),(Xao,Cc,YZbo),(Xao,Cc,YZbp),(Xao,Cc,YZbq),(Xao, Cc,YZbr),(Xao,Cc,YZbs),(Xao,Cc,YZbt),(Xao,Cc,YZbu),(Xao,Cc,YZbv),(Xao,Cc,YZbw), (Xao,Cc,YZbx),(Xao,Cc,YZby),(Xao,Cc,YZbz),(Xao,Cc,YZca),(Xao,Cc,YZcb),(Xao, Cc,YZcc),(Xao,Cc,YZcd),(Xao,Cc,YZce),(Xao,Cc,YZcf),(Xao,Cc,YZcg),(Xao,Cc, YZch),(Xao,Cc,YZci),(Xao,Cc,YZcj),(Xao,Cc,YZck),(Xao,Cc,YZcl),(Xao,Cc,YZcm), (Xao,Cc,YZcn),(Xao,Cc,YZco),(Xao,Cc,YZcp),(Xao,Cc,YZcq),(Xao,Cc,YZcr),(Xao, Cc,YZcs),(Xao,Cc,YZct),(Xao,Cc,YZcu),(Xao,Cc,YZcv),(Xao,Cc,YZcw),(Xao,Cc,YZcx), (Xao,Cc,YZcy),(Xao,Cc,YZcz),(Xao,Cc,YZda),(Xao,Cc,YZdb),(Xao,Cc,YZdc),(Xao, Cc,YZdd),(Xao,Cc,YZde),(Xao,Cc,YZdf),(Xao,Cc,YZdg),(Xao,Cc,YZdh),(Xao,Cc, YZdi),(Xao,Cc,YZdj),(Xao,Cc,YZdk),(Xao,Cc,YZdl),(Xao,Cc,YZdm),(Xao,Cc,YZdn), (Xao,Cc,YZdo),(Xao,Cc,YZdp),(Xao,Cc,YZdq),(Xao,Cc,YZdr),(Xao,Cc,YZds),(Xao, Cc,Yzdt),(Xao,Cc,YZdu),(Xao,Cc,YZdv),(Xao,Cc,YZdw),(Xao,Cc,YZdx),(Xao,Cc, YZdy),(Xao,Cc,YZdz),(Xao,Cc,YZea),(Xao,Cc,YZeb),(Xao,Cc,YZec),(Xao,Cc,YZed), (Xao,Cc,YZee),(Xao,Cc,YZef),(Xao,Cc,YZeg),(Xao,Cc,YZeh),(Xao,Cc,YZei),(Xao, Cc,YZej),(Xao,Cc,YZek),(Xao,Cc,YZel),(Xao,Cc,YZem),(Xao,Cc,YZen),(Xao,Cc,YZeo), (Xao,Cc,YZep),(Xao,Cc,YZeq),(Xao,Cc,YZer),(Xao,Cc,YZes),(Xao,Cc,YZet),(Xao, Cc,YZeu),(Xao,Cc,YZev),(Xao,Cc,YZew),(Xao,Cc,YZex),(Xao,Cc,YZey),(Xao,Cc, YZez),(Xao,Cc,YZfa),(Xao,Cc,YZfb),(Xao,Cc,YZfc),(Xao,Cc,YZfd),(Xao,Cc,YZfe),(Xao, Cc,YZff),(Xao,Cc,YZfg),(Xao,Cc,YZfh),(Xao,Cc,YZfi),(Xao,Cc,YZfj),(Xao,Cc, YZfk),(Xao,Cc,YZfl),(Xao,Cc,YZfm),(Xao,Cc,YZfn),(Xao,Cc,YZfo),(Xao,Cc,YZfp),(Xao, Cc,YZfq),(Xao,Cc,YZfr),(Xao,Cc,YZfs),(Xao,Cc,YZft),(Xao,Cc,YZfu),(Xao,Cc, YZfv),(Xao,Cc,YZfw),(Xao,Cc,YZfx),(Xao,Cc,YZfy),(Xao,Cc,YZfz),(Xao,Cc,YZga), (Xao,Cc,YZgb),(Xao,Cc,YZgc),(Xao,Cc,YZgd),(Xao,Cc,YZge),(Xao,Cc,YZgf),(Xao, Cc,YZgg),(Xao,Cc,YZgh),(Xao,Cc,YZgi),(Xao,Cc,YZgj),(Xao,Cc,YZgk),(Xao,Cc,YZgl), (Xao,Cc,YZgm),(Xao,Cc,YZgn),(Xao,Cc,YZgo),(Xao,Cc,YZgp),(Xao,Cc,YZgq),(Xao, Cc,YZgr),(Xao,Cc,YZgs),(Xao,Cc,YZgt),(Xao,Cd,YZa),(Xao,Cd,YZb),(Xao,Cd, YZc),(Xao,Cd,YZd),(Xao,Cd,YZe),(Xao,Cd,YZf),(Xao,Cd,YZg),(Xao,Cd,YZh),(Xao, Cd,YZi),(Xao,Cd,YZj),(Xao,Cd,YZk),(Xao,Cd,YZl),(Xao,Cd,YZm),(Xao,Cd,YZn),(Xao, Cd,YZo),(Xao,Cd,YZp),(Xao,Cd,YZq),(Xao,Cd,YZr),(Xao,Cd,YZs),(Xao,Cd,YZt), (Xao,Cd,YZu),(Xao,Cd,YZv),(Xao,Cd,YZw),(Xao,Cd,YZx),(Xao,Cd,YZy),(Xao,Cd, YZz),(Xao,Cd,YZaa),(Xao,Cd,YZab),(Xao,Cd,YZac),(Xao,Cd,YZad),(Xao,Cd,YZae), (Xao,Cd,YZaf),(Xao,Cd,YZag),(Xao,Cd,YZah),(Xao,Cd,YZai),(Xao,Cd,YZaj),(Xao,Cd, YZak),(Xao,Cd,YZal),(Xao,Cd,YZam),(Xao,Cd,YZan),(Xao,Cd,YZao),(Xao,Cd,YZap), (Xao,Cd,YZaq),(Xao,Cd,YZar),(Xao,Cd,YZas),(Xao,Cd,YZat),(Xao,Cd,YZau),(Xao, Cd,YZav),(Xao,Cd,YZaw),(Xao,Cd,YZax),(Xao,Cd,YZay),(Xao,Cd,YZaz),(Xao,Cd, YZba),(Xao,Cd,YZbb),(Xao,Cd,YZbc),(Xao,Cd,YZbd),(Xao,Cd,YZbe),(Xao,Cd,YZbf), (Xao,Cd,YZbg),(Xao,Cd,YZbh),(Xao,Cd,YZbi),(Xao,Cd,YZbj),(Xao,Cd,YZbk),(Xao, Cd,YZbl),(Xao,Cd,YZbm),(Xao,Cd,YZbn),(Xao,Cd,YZbo),(Xao,Cd,YZbp),(Xao,Cd, YZbq),(Xao,Cd,YZbr),(Xao,Cd,YZbs),(Xao,Cd,YZbt),(Xao,Cd,YZbu),(Xao,Cd,YZbv), (Xao,Cd,YZbw),(Xao,Cd,YZbx),(Xao,Cd,YZby),(Xao,Cd,YZbz),(Xao,Cd,YZca),(Xao, Cd,YZcb),(Xao,Cd,YZcc),(Xao,Cd,YZcd),(Xao,Cd,YZce),(Xao,Cd,YZcf),(Xao,Cd, YZcg),(Xao,Cd,YZch),(Xao,Cd,YZci),(Xao,Cd,YZcj),(Xao,Cd,YZck),(Xao,Cd,YZcl), (Xao,Cd,YZcm),(Xao,Cd,YZcn),(Xao,Cd,YZco),(Xao,Cd,YZcp),(Xao,Cd,YZcq),(Xao, Cd,YZcr),(Xao,Cd,YZcs),(Xao,Cd,YZct),(Xao,Cd,YZcu),(Xao,Cd,YZcv),(Xao,Cd, YZcw),(Xao,Cd,YZcx),(Xao,Cd,YZcy),(Xao,Cd,YZcz),(Xao,Cd,YZda),(Xao,Cd,YZdb), (Xao,Cd,YZdc),(Xao,Cd,YZdd),(Xao,Cd,YZde),(Xao,Cd,YZdf),(Xao,Cd,YZdg),(Xao, Cd,YZdh),(Xao,Cd,YZdi),(Xao,Cd,YZdj),(Xao,Cd,YZdk),(Xao,Cd,YZdl),(Xao,Cd, YZdm),(Xao,Cd,YZdn),(Xao,Cd,YZdo),(Xao,Cd,YZdp),(Xao,Cd,YZdq),(Xao,Cd,YZdr), (Xao,Cd,YZds),(Xao,Cd,Yzdt),(Xao,Cd,YZdu),(Xao,Cd,YZdv),(Xao,Cd,YZdw),(Xao, Cd,YZdx),(Xao,Cd,YZdy),(Xao,Cd,YZdz),(Xao,Cd,YZea),(Xao,Cd,YZeb),(Xao, Cd,YZec),(Xao,Cd,YZed),(Xao,Cd,YZee),(Xao,Cd,YZef),(Xao,Cd,YZeg),(Xao,Cd,YZeh), (Xao,Cd,YZei),(Xao,Cd,YZej),(Xao,Cd,YZek),(Xao,Cd,YZel),(Xao,Cd,YZem),(Xao, Cd,YZen),(Xao,Cd,YZeo),(Xao,Cd,YZep),(Xao,Cd,YZeq),(Xao,Cd,YZer),(Xao, Cd,YZes),(Xao,Cd,YZet),(Xao,Cd,YZeu),(Xao,Cd,YZev),(Xao,Cd,YZew),(Xao,Cd,YZex), (Xao,Cd,YZey),(Xao,Cd,YZez),(Xao,Cd,YZfa),(Xao,Cd,YZfb),(Xao,Cd,YZfc),(Xao, Cd,YZfd),(Xao,Cd,YZfe),(Xao,Cd,YZff),(Xao,Cd,YZfg),(Xao,Cd,YZfh),(Xao,Cd, YZfi),(Xao,Cd,YZfj),(Xao,Cd,YZfk),(Xao,Cd,YZfl),(Xao,Cd,YZfm),(Xao,Cd,YZfn), (Xao,Cd,YZfo),(Xao,Cd,YZfp),(Xao,Cd,YZfq),(Xao,Cd,YZfr),(Xao,Cd,YZfs),(Xao,Cd, YZft),(Xao,Cd,YZfu),(Xao,Cd,YZfv),(Xao,Cd,YZfw),(Xao,Cd,YZfx),(Xao,Cd,YZfy), (Xao,Cd,YZfz),(Xao,Cd,YZga),(Xao,Cd,YZgb),(Xao,Cd,YZgc),(Xao,Cd,YZgd),(Xao, Cd,YZge),(Xao,Cd,YZgf),(Xao,Cd,YZgg),(Xao,Cd,YZgh),(Xao,Cd,YZgi),(Xao,Cd, YZgj),(Xao,Cd,YZgk),(Xao,Cd,YZgl),(Xao,Cd,YZgm),(Xao,Cd,YZgn),(Xao,Cd,YZgo), (Xao,Cd,YZgp),(Xao,Cd,YZgq),(Xao,Cd,YZgr),(Xao,Cd,YZgs),(Xao,Cd,YZgt),(Xao, Ce,YZa),(Xao,Ce,YZb),(Xao,Ce,YZc),(Xao,Ce,YZd),(Xao,Ce,YZe),(Xao,Ce,YZf),(Xao, Ce,YZg),(Xao,Ce,YZh),(Xao,Ce,YZi),(Xao,Ce,YZj),(Xao,Ce,YZk),(Xao,Ce,YZl), (Xao,Ce,YZm),(Xao,Ce,YZn),(Xao,Ce,YZo),(Xao,Ce,YZp),(Xao,Ce,YZq),(Xao,Ce,YZr), (Xao,Ce,YZs),(Xao,Ce,YZt),(Xao,Ce,YZu),(Xao,Ce,YZv),(Xao,Ce,YZw),(Xao,Ce, YZx),(Xao,Ce,YZy),(Xao,Ce,YZz),(Xao,Ce,YZaa),(Xao,Ce,YZab),(Xao,Ce,YZac),(Xao, Ce,YZad),(Xao,Ce,YZae),(Xao,Ce,YZaf),(Xao,Ce,YZag),(Xao,Ce,YZah),(Xao,Ce, YZai),(Xao,Ce,YZaj),(Xao,Ce,YZak),(Xao,Ce,YZal),(Xao,Ce,YZam),(Xao,Ce,YZan), (Xao,Ce,YZao),(Xao,Ce,YZap),(Xao,Ce,YZaq),(Xao,Ce,YZar),(Xao,Ce,YZas),(Xao, Ce,YZat),(Xao,Ce,YZau),(Xao,Ce,YZav),(Xao,Ce,YZaw),(Xao,Ce,YZax),(Xao,Ce,YZay), (Xao,Ce,YZaz),(Xao,Ce,YZba),(Xao,Ce,YZbb),(Xao,Ce,YZbc),(Xao,Ce,YZbd),(Xao, Ce,YZbe),(Xao,Ce,YZbf),(Xao,Ce,YZbg),(Xao,Ce,YZbh),(Xao,Ce,YZbi),(Xao,Ce, YZbj),(Xao,Ce,YZbk),(Xao,Ce,YZbl),(Xao,Ce,YZbm),(Xao,Ce,YZbn),(Xao,Ce,YZbo), (Xao,Ce,YZbp),(Xao,Ce,YZbq),(Xao,Ce,YZbr),(Xao,Ce,YZbs),(Xao,Ce,YZbt),(Xao, Ce,YZbu),(Xao,Ce,YZbv),(Xao,Ce,YZbw),(Xao,Ce,YZbx),(Xao,Ce,YZby),(Xao,Ce, YZbz),(Xao,Ce,YZca),(Xao,Ce,YZcb),(Xao,Ce,YZcc),(Xao,Ce,YZcd),(Xao,Ce,YZce), (Xao,Ce,YZcf),(Xao,Ce,YZcg),(Xao,Ce,YZch),(Xao,Ce,YZci),(Xao,Ce,YZcj),(Xao,Ce, YZck),(Xao,Ce,YZcl),(Xao,Ce,YZcm),(Xao,Ce,YZcn),(Xao,Ce,YZco),(Xao,Ce,YZcp), (Xao,Ce,YZcq),(Xao,Ce,YZcr),(Xao,Ce,YZcs),(Xao,Ce,YZct),(Xao,Ce,YZcu),(Xao, Ce,YZcv),(Xao,Ce,YZcw),(Xao,Ce,YZcx),(Xao,Ce,YZcy),(Xao,Ce,YZcz),(Xao,Ce,YZda), (Xao,Ce,YZdb),(Xao,Ce,YZdc),(Xao,Ce,YZdd),(Xao,Ce,YZde),(Xao,Ce,YZdf),(Xao, Ce,YZdg),(Xao,Ce,YZdh),(Xao,Ce,YZdi),(Xao,Ce,YZdj),(Xao,Ce,YZdk),(Xao,Ce, YZdl),(Xao,Ce,YZdm),(Xao,Ce,YZdn),(Xao,Ce,YZdo),(Xao,Ce,YZdp),(Xao,Ce,YZdq), (Xao,Ce,YZdr),(Xao,Ce,YZds),(Xao,Ce,Yzdt),(Xao,Ce,YZdu),(Xao,Ce,YZdv),(Xao, Ce,YZdw),(Xao,Ce,YZdx),(Xao,Ce,YZdy),(Xao,Ce,YZdz),(Xao,Ce,YZea),(Xao,Ce, YZeb),(Xao,Ce,YZec),(Xao,Ce,YZed),(Xao,Ce,YZee),(Xao,Ce,YZef),(Xao,Ce,YZeg), (Xao,Ce,YZeh),(Xao,Ce,YZei),(Xao,Ce,YZej),(Xao,Ce,YZek),(Xao,Ce,YZel),(Xao,Ce, YZem),(Xao,Ce,YZen),(Xao,Ce,YZeo),(Xao,Ce,YZep),(Xao,Ce,YZeq),(Xao,Ce,YZer), (Xao,Ce,YZes),(Xao,Ce,YZet),(Xao,Ce,YZeu),(Xao,Ce,YZev),(Xao,Ce,YZew),(Xao, Ce,YZex),(Xao,Ce,YZey),(Xao,Ce,YZez),(Xao,Ce,YZfa),(Xao,Ce,YZfb),(Xao,Ce, YZfc),(Xao,Ce,YZfd),(Xao,Ce,YZfe),(Xao,Ce,YZff),(Xao,Ce,YZfg),(Xao,Ce,YZfh),(Xao, Ce,YZfi),(Xao,Ce,YZfj),(Xao,Ce,YZfk),(Xao,Ce,YZfl),(Xao,Ce,YZfm),(Xao,Ce, YZfn),(Xao,Ce,YZfo),(Xao,Ce,YZfp),(Xao,Ce,YZfq),(Xao,Ce,YZfr),(Xao,Ce,YZfs),(Xao, Ce,YZft),(Xao,Ce,YZfu),(Xao,Ce,YZfv),(Xao,Ce,YZfw),(Xao,Ce,YZfx),(Xao,Ce, YZfy),(Xao,Ce,YZfz),(Xao,Ce,YZga),(Xao,Ce,YZgb),(Xao,Ce,YZgc),(Xao,Ce,YZgd), (Xao,Ce,YZge),(Xao,Ce,YZgf),(Xao,Ce,YZgg),(Xao,Ce,YZgh),(Xao,Ce,YZgi),(Xao, Ce,YZgj),(Xao,Ce,YZgk),(Xao,Ce,YZgl),(Xao,Ce,YZgm),(Xao,Ce,YZgn),(Xao,Ce,YZgo), (Xao,Ce,YZgp),(Xao,Ce,YZgq),(Xao,Ce,YZgr),(Xao,Ce,YZgs),(Xao,Ce,YZgt),(Xao, Cf,YZa),(Xao,Cf,YZb),(Xao,Cf,YZc),(Xao,Cf,YZd),(Xao,Cf,YZe),(Xao,Cf,YZf), (Xao,Cf,YZg),(Xao,Cf,YZh),(Xao,Cf,YZi),(Xao,Cf,YZj),(Xao,Cf,YZk),(Xao,Cf,YZl), (Xao,Cf,YZm),(Xao,Cf,YZn),(Xao,Cf,YZo),(Xao,Cf,YZp),(Xao,Cf,YZq),(Xao,Cf,YZr), (Xao,Cf,YZs),(Xao,Cf,YZt),(Xao,Cf,YZu),(Xao,Cf,YZv),(Xao,Cf,YZw),(Xao,Cf,YZx), (Xao,Cf,YZy),(Xao,Cf,YZz),(Xao,Cf,YZaa),(Xao,Cf,YZab),(Xao,Cf,YZac),(Xao, Cf,YZad),(Xao,Cf,YZae),(Xao,Cf,YZaf),(Xao,Cf,YZag),(Xao,Cf,YZah),(Xao,Cf,YZai), (Xao,Cf,YZaj),(Xao,Cf,YZak),(Xao,Cf,YZal),(Xao,Cf,YZam),(Xao,Cf,YZan),(Xao, Cf,YZao),(Xao,Cf,YZap),(Xao,Cf,YZaq),(Xao,Cf,YZar),(Xao,Cf,YZas),(Xao,Cf,YZat), (Xao,Cf,YZau),(Xao,Cf,YZav),(Xao,Cf,YZaw),(Xao,Cf,YZax),(Xao,Cf,YZay),(Xao, Cf,YZaz),(Xao,Cf,YZba),(Xao,Cf,YZbb),(Xao,Cf,YZbc),(Xao,Cf,YZbd),(Xao,Cf,YZbe), (Xao,Cf,YZbf),(Xao,Cf,YZbg),(Xao,Cf,YZbh),(Xao,Cf,YZbi),(Xao,Cf,YZbj),(Xao, Cf,YZbk),(Xao,Cf,YZbl),(Xao,Cf,YZbm),(Xao,Cf,YZbn),(Xao,Cf,YZbo),(Xao,Cf,YZbp), (Xao,Cf,YZbq),(Xao,Cf,YZbr),(Xao,Cf,YZbs),(Xao,Cf,YZbt),(Xao,Cf,YZbu),(Xao, Cf,YZbv),(Xao,Cf,YZbw),(Xao,Cf,YZbx),(Xao,Cf,YZby),(Xao,Cf,YZbz),(Xao,Cf,YZca), (Xao,Cf,YZcb),(Xao,Cf,YZcc),(Xao,Cf,YZcd),(Xao,Cf,YZce),(Xao,Cf,YZcf),(Xao, Cf,YZcg),(Xao,Cf,YZch),(Xao,Cf,YZci),(Xao,Cf,YZcj),(Xao,Cf,YZck),(Xao,Cf,YZcl), (Xao,Cf,YZcm),(Xao,Cf,YZcn),(Xao,Cf,YZco),(Xao,Cf,YZcp),(Xao,Cf,YZcq),(Xao, Cf,YZcr),(Xao,Cf,YZcs),(Xao,Cf,YZct),(Xao,Cf,YZcu),(Xao,Cf,YZcv),(Xao,Cf, YZcw),(Xao,Cf,YZcx),(Xao,Cf,YZcy),(Xao,Cf,YZcz),(Xao,Cf,YZda),(Xao,Cf,YZdb), (Xao,Cf,YZdc),(Xao,Cf,YZdd),(Xao,Cf,YZde),(Xao,Cf,YZdf),(Xao,Cf,YZdg),(Xao,Cf, YZdh),(Xao,Cf,YZdi),(Xao,Cf,YZdj),(Xao,Cf,YZdk),(Xao,Cf,YZdl),(Xao,Cf,YZdm), (Xao,Cf,YZdn),(Xao,Cf,YZdo),(Xao,Cf,YZdp),(Xao,Cf,YZdq),(Xao,Cf,YZdr),(Xao, Cf,YZds),(Xao,Cf,Yzdt),(Xao,Cf,YZdu),(Xao,Cf,YZdv),(Xao,Cf,YZdw),(Xao,Cf,YZdx), (Xao,Cf,YZdy),(Xao,Cf,YZdz),(Xao,Cf,YZea),(Xao,Cf,YZeb),(Xao,Cf,YZec),(Xao, Cf,YZed),(Xao,Cf,YZee),(Xao,Cf,YZef),(Xao,Cf,YZeg),(Xao,Cf,YZeh),(Xao,Cf,YZei), (Xao,Cf,YZej),(Xao,Cf,YZek),(Xao,Cf,YZel),(Xao,Cf,YZem),(Xao,Cf,YZen),(Xao, Cf,YZeo),(Xao,Cf,YZep),(Xao,Cf,YZeq),(Xao,Cf,YZer),(Xao,Cf,YZes),(Xao,Cf,YZet), (Xao,Cf,YZeu),(Xao,Cf,YZev),(Xao,Cf,YZew),(Xao,Cf,YZex),(Xao,Cf,YZey),(Xao, Cf,YZez),(Xao,Cf,YZfa),(Xao,Cf,YZfb),(Xao,Cf,YZfc),(Xao,Cf,YZfd),(Xao,Cf,YZfe), (Xao,Cf,YZff),(Xao,Cf,YZfg),(Xao,Cf,YZfh),(Xao,Cf,YZfi),(Xao,Cf,YZfj),(Xao,Cf, YZfk),(Xao,Cf,YZfl),(Xao,Cf,YZfm),(Xao,Cf,YZfn),(Xao,Cf,YZfo),(Xao,Cf,YZfp),(Xao, Cf,YZfq),(Xao,Cf,YZfr),(Xao,Cf,YZfs),(Xao,Cf,YZft),(Xao,Cf,YZfu),(Xao,Cf,YZfv), (Xao,Cf,YZfw),(Xao,Cf,YZfx),(Xao,Cf,YZfy),(Xao,Cf,YZfz),(Xao,Cf,YZga),(Xao, Cf,YZgb),(Xao,Cf,YZgc),(Xao,Cf,YZgd),(Xao,Cf,YZge),(Xao,Cf,YZgf),(Xao,Cf,YZgg), (Xao,Cf,YZgh),(Xao,Cf,YZgi),(Xao,Cf,YZgj),(Xao,Cf,YZgk),(Xao,Cf,YZgl),(Xao, Cf,YZgm),(Xao,Cf,YZgn),(Xao,Cf,YZgo),(Xao,Cf,YZgp),(Xao,Cf,YZgq),(Xao,Cf, YZgr),(Xao,Cf,YZgs),(Xao,Cf,YZgt), (Xap,Ca,YZa),(Xap,Ca,YZb),(Xap,Ca,YZc),(Xap,Ca,YZd),(Xap,Ca,YZe),(Xap,Ca,YZf), (Xap,Ca,YZg),(Xap,Ca,YZh),(Xap,Ca,YZi),(Xap,Ca,YZj),(Xap,Ca,YZk),(Xap,Ca,YZl), (Xap,Ca,YZm),(Xap,Ca,YZn),(Xap,Ca,YZo),(Xap,Ca,YZp),(Xap,Ca,YZq),(Xap,Ca, YZr),(Xap,Ca,YZs),(Xap,Ca,YZt),(Xap,Ca,YZu),(Xap,Ca,YZv),(Xap,Ca,YZw),(Xap, Ca,YZx),(Xap,Ca,YZy),(Xap,Ca,YZz),(Xap,Ca,YZaa),(Xap,Ca,YZab),(Xap,Ca,YZac), (Xap,Ca,YZad),(Xap,Ca,YZae),(Xap,Ca,YZaf),(Xap,Ca,YZag),(Xap,Ca,YZah),(Xap, Ca,YZai),(Xap,Ca,YZaj),(Xap,Ca,YZak),(Xap,Ca,YZal),(Xap,Ca,YZam),(Xap,Ca,YZan), (Xap,Ca,YZao),(Xap,Ca,YZap),(Xap,Ca,YZaq),(Xap,Ca,YZar),(Xap,Ca,YZas),(Xap, Ca,YZat),(Xap,Ca,YZau),(Xap,Ca,YZav),(Xap,Ca,YZaw),(Xap,Ca,YZax),(Xap,Ca, YZay),(Xap,Ca,YZaz),(Xap,Ca,YZba),(Xap,Ca,YZbb),(Xap,Ca,YZbc),(Xap,Ca,YZbd), (Xap,Ca,YZbe),(Xap,Ca,YZbf),(Xap,Ca,YZbg),(Xap,Ca,YZbh),(Xap,Ca,YZbi),(Xap, Ca,YZbj),(Xap,Ca,YZbk),(Xap,Ca,YZbl),(Xap,Ca,YZbm),(Xap,Ca,YZbn),(Xap,Ca,YZbo), (Xap,Ca,YZbp),(Xap,Ca,YZbq),(Xap,Ca,YZbr),(Xap,Ca,YZbs),(Xap,Ca,YZbt),(Xap, Ca,YZbu),(Xap,Ca,YZbv),(Xap,Ca,YZbw),(Xap,Ca,YZbx),(Xap,Ca,YZby),(Xap, Ca,YZbz),(Xap,Ca,YZca),(Xap,Ca,YZcb),(Xap,Ca,YZcc),(Xap,Ca,YZcd),(Xap,Ca,YZce), (Xap,Ca,YZcf),(Xap,Ca,YZcg),(Xap,Ca,YZch),(Xap,Ca,YZci),(Xap,Ca,YZcj),(Xap, Ca,YZck),(Xap,Ca,YZcl),(Xap,Ca,YZcm),(Xap,Ca,YZcn),(Xap,Ca,YZco),(Xap,Ca, YZcp),(Xap,Ca,YZcq),(Xap,Ca,YZcr),(Xap,Ca,YZcs),(Xap,Ca,YZct),(Xap,Ca,YZcu), (Xap,Ca,YZcv),(Xap,Ca,YZcw),(Xap,Ca,YZcx),(Xap,Ca,YZcy),(Xap,Ca,YZcz),(Xap, Ca,YZda),(Xap,Ca,YZdb),(Xap,Ca,YZdc),(Xap,Ca,YZdd),(Xap,Ca,YZde),(Xap,Ca,YZdf), (Xap,Ca,YZdg),(Xap,Ca,YZdh),(Xap,Ca,YZdi),(Xap,Ca,YZdj),(Xap,Ca,YZdk),(Xap, Ca,YZdl),(Xap,Ca,YZdm),(Xap,Ca,YZdn),(Xap,Ca,YZdo),(Xap,Ca,YZdp),(Xap, Ca,YZdq),(Xap,Ca,YZdr),(Xap,Ca,YZds),(Xap,Ca,Yzdt),(Xap,Ca,YZdu),(Xap,Ca,YZdv), (Xap,Ca,YZdw),(Xap,Ca,YZdx),(Xap,Ca,YZdy),(Xap,Ca,YZdz),(Xap,Ca,YZea),(Xap, Ca,YZeb),(Xap,Ca,YZec),(Xap,Ca,YZed),(Xap,Ca,YZee),(Xap,Ca,YZef),(Xap,Ca, YZeg),(Xap,Ca,YZeh),(Xap,Ca,YZei),(Xap,Ca,YZej),(Xap,Ca,YZek),(Xap,Ca,YZel), (Xap,Ca,YZem),(Xap,Ca,YZen),(Xap,Ca,YZeo),(Xap,Ca,YZep),(Xap,Ca,YZeq),(Xap, Ca,YZer),(Xap,Ca,YZes),(Xap,Ca,YZet),(Xap,Ca,YZeu),(Xap,Ca,YZev),(Xap,Ca,YZew), (Xap,Ca,YZex),(Xap,Ca,YZey),(Xap,Ca,YZez),(Xap,Ca,YZfa),(Xap,Ca,YZfb),(Xap, Ca,YZfc),(Xap,Ca,YZfd),(Xap,Ca,YZfe),(Xap,Ca,YZff),(Xap,Ca,YZfg),(Xap,Ca,YZfh), (Xap,Ca,YZfi),(Xap,Ca,YZfj),(Xap,Ca,YZfk),(Xap,Ca,YZfl),(Xap,Ca,YZfm),(Xap, Ca,YZfn),(Xap,Ca,YZfo),(Xap,Ca,YZfp),(Xap,Ca,YZfq),(Xap,Ca,YZfr),(Xap,Ca,YZfs), (Xap,Ca,YZft),(Xap,Ca,YZfu),(Xap,Ca,YZfv),(Xap,Ca,YZfw),(Xap,Ca,YZfx),(Xap, Ca,YZfy),(Xap,Ca,YZfz),(Xap,Ca,YZga),(Xap,Ca,YZgb),(Xap,Ca,YZgc),(Xap,Ca, YZgd),(Xap,Ca,YZge),(Xap,Ca,YZgf),(Xap,Ca,YZgg),(Xap,Ca,YZgh),(Xap,Ca,YZgi), (Xap,Ca,YZgj),(Xap,Ca,YZgk),(Xap,Ca,YZgl),(Xap,Ca,YZgm),(Xap,Ca,YZgn),(Xap, Ca,YZgo),(Xap,Ca,YZgp),(Xap,Ca,YZgq),(Xap,Ca,YZgr),(Xap,Ca,YZgs),(Xap,Ca,YZgt), (Xap,Cb,YZa),(Xap,Cb,YZb),(Xap,Cb,YZc),(Xap,Cb,YZd),(Xap,Cb,YZe),(Xap, Cb,YZf),(Xap,Cb,YZg),(Xap,Cb,YZh),(Xap,Cb,YZi),(Xap,Cb,YZj),(Xap,Cb,YZk),(Xap, Cb,YZl),(Xap,Cb,YZm),(Xap,Cb,YZn),(Xap,Cb,YZo),(Xap,Cb,YZp),(Xap,Cb,YZq), (Xap,Cb,YZr),(Xap,Cb,YZs),(Xap,Cb,YZt),(Xap,Cb,YZu),(Xap,Cb,YZv),(Xap,Cb,YZw), (Xap,Cb,YZx),(Xap,Cb,YZy),(Xap,Cb,YZz),(Xap,Cb,YZaa),(Xap,Cb,YZab),(Xap, Cb,YZac),(Xap,Cb,YZad),(Xap,Cb,YZae),(Xap,Cb,YZaf),(Xap,Cb,YZag),(Xap,Cb, YZah),(Xap,Cb,YZai),(Xap,Cb,YZaj),(Xap,Cb,YZak),(Xap,Cb,YZal),(Xap,Cb,YZam), (Xap,Cb,YZan),(Xap,Cb,YZao),(Xap,Cb,YZap),(Xap,Cb,YZaq),(Xap,Cb,YZar),(Xap, Cb,YZas),(Xap,Cb,YZat),(Xap,Cb,YZau),(Xap,Cb,YZav),(Xap,Cb,YZaw),(Xap,Cb,YZax), (Xap,Cb,YZay),(Xap,Cb,YZaz),(Xap,Cb,YZba),(Xap,Cb,YZbb),(Xap,Cb,YZbc), (Xap,Cb,YZbd),(Xap,Cb,YZbe),(Xap,Cb,YZbf),(Xap,Cb,YZbg),(Xap,Cb,YZbh),(Xap, Cb,YZbi),(Xap,Cb,YZbj),(Xap,Cb,YZbk),(Xap,Cb,YZbl),(Xap,Cb,YZbm),(Xap,Cb,YZbn), (Xap,Cb,YZbo),(Xap,Cb,YZbp),(Xap,Cb,YZbq),(Xap,Cb,YZbr),(Xap,Cb,YZbs), (Xap,Cb,YZbt),(Xap,Cb,YZbu),(Xap,Cb,YZbv),(Xap,Cb,YZbw),(Xap,Cb,YZbx),(Xap, Cb,YZby),(Xap,Cb,YZbz),(Xap,Cb,YZca),(Xap,Cb,YZcb),(Xap,Cb,YZcc),(Xap,Cb,YZcd), (Xap,Cb,YZce),(Xap,Cb,YZcf),(Xap,Cb,YZcg),(Xap,Cb,YZch),(Xap,Cb,YZci),(Xap, Cb,YZcj),(Xap,Cb,YZck),(Xap,Cb,YZcl),(Xap,Cb,YZcm),(Xap,Cb,YZcn),(Xap, Cb,YZco),(Xap,Cb,YZcp),(Xap,Cb,YZcq),(Xap,Cb,YZcr),(Xap,Cb,YZcs),(Xap,Cb,YZct), (Xap,Cb,YZcu),(Xap,Cb,YZcv),(Xap,Cb,YZcw),(Xap,Cb,YZcx),(Xap,Cb,YZcy), (Xap,Cb,YZcz),(Xap,Cb,YZda),(Xap,Cb,YZdb),(Xap,Cb,YZdc),(Xap,Cb,YZdd),(Xap, Cb,YZde),(Xap,Cb,YZdf),(Xap,Cb,YZdg),(Xap,Cb,YZdh),(Xap,Cb,YZdi),(Xap,Cb,YZdj), (Xap,Cb,YZdk),(Xap,Cb,YZdl),(Xap,Cb,YZdm),(Xap,Cb,YZdn),(Xap,Cb,YZdo), (Xap,Cb,YZdp),(Xap,Cb,YZdq),(Xap,Cb,YZdr),(Xap,Cb,YZds),(Xap,Cb,Yzdt),(Xap, Cb,YZdu),(Xap,Cb,YZdv),(Xap,Cb,YZdw),(Xap,Cb,YZdx),(Xap,Cb,YZdy),(Xap,Cb, YZdz),(Xap,Cb,YZea),(Xap,Cb,YZeb),(Xap,Cb,YZec),(Xap,Cb,YZed),(Xap,Cb,YZee), (Xap,Cb,YZef),(Xap,Cb,YZeg),(Xap,Cb,YZeh),(Xap,Cb,YZei),(Xap,Cb,YZej),(Xap, Cb,YZek),(Xap,Cb,YZel),(Xap,Cb,YZem),(Xap,Cb,YZen),(Xap,Cb,YZeo),(Xap,Cb,YZep), (Xap,Cb,YZeq),(Xap,Cb,YZer),(Xap,Cb,YZes),(Xap,Cb,YZet),(Xap,Cb,YZeu),(Xap, Cb,YZev),(Xap,Cb,YZew),(Xap,Cb,YZex),(Xap,Cb,YZey),(Xap,Cb,YZez),(Xap, Cb,YZfa),(Xap,Cb,YZfb),(Xap,Cb,YZfc),(Xap,Cb,YZfd),(Xap,Cb,YZfe),(Xap,Cb,YZff), (Xap,Cb,YZfg),(Xap,Cb,YZfh),(Xap,Cb,YZfi),(Xap,Cb,YZfj),(Xap,Cb,YZfk),(Xap, Cb,YZfl),(Xap,Cb,YZfm),(Xap,Cb,YZfn),(Xap,Cb,YZfo),(Xap,Cb,YZfp),(Xap,Cb,YZfq), (Xap,Cb,YZfr),(Xap,Cb,YZfs),(Xap,Cb,YZft),(Xap,Cb,YZfu),(Xap,Cb,YZfv),(Xap, Cb,YZfw),(Xap,Cb,YZfx),(Xap,Cb,YZfy),(Xap,Cb,YZfz),(Xap,Cb,YZga),(Xap,Cb,YZgb), (Xap,Cb,YZgc),(Xap,Cb,YZgd),(Xap,Cb,YZge),(Xap,Cb,YZgf),(Xap,Cb,YZgg), (Xap,Cb,YZgh),(Xap,Cb,YZgi),(Xap,Cb,YZgj),(Xap,Cb,YZgk),(Xap,Cb,YZgl),(Xap, Cb,YZgm),(Xap,Cb,YZgn),(Xap,Cb,YZgo),(Xap,Cb,YZgp),(Xap,Cb,YZgq),(Xap,Cb, YZgr),(Xap,Cb,YZgs),(Xap,Cb,YZgt),(Xap,Cc,YZa),(Xap,Cc,YZb),(Xap,Cc,YZc),(Xap, Cc,YZd),(Xap,Cc,YZe),(Xap,Cc,YZf),(Xap,Cc,YZg),(Xap,Cc,YZh),(Xap,Cc,YZi),(Xap, Cc,YZj),(Xap,Cc,YZk),(Xap,Cc,YZl),(Xap,Cc,YZm),(Xap,Cc,YZn),(Xap,Cc,YZo), (Xap,Cc,YZp),(Xap,Cc,YZq),(Xap,Cc,YZr),(Xap,Cc,YZs),(Xap,Cc,YZt),(Xap,Cc, YZu),(Xap,Cc,YZv),(Xap,Cc,YZw),(Xap,Cc,YZx),(Xap,Cc,YZy),(Xap,Cc,YZz),(Xap, Cc,YZaa),(Xap,Cc,YZab),(Xap,Cc,YZac),(Xap,Cc,YZad),(Xap,Cc,YZae),(Xap,Cc,YZaf), (Xap,Cc,YZag),(Xap,Cc,YZah),(Xap,Cc,YZai),(Xap,Cc,YZaj),(Xap,Cc,YZak),(Xap, Cc,YZal),(Xap,Cc,YZam),(Xap,Cc,YZan),(Xap,Cc,YZao),(Xap,Cc,YZap),(Xap,Cc, YZaq),(Xap,Cc,YZar),(Xap,Cc,YZas),(Xap,Cc,YZat),(Xap,Cc,YZau),(Xap,Cc,YZav), (Xap,Cc,YZaw),(Xap,Cc,YZax),(Xap,Cc,YZay),(Xap,Cc,YZaz),(Xap,Cc,YZba),(Xap, Cc,YZbb),(Xap,Cc,YZbc),(Xap,Cc,YZbd),(Xap,Cc,YZbe),(Xap,Cc,YZbf),(Xap,Cc,YZbg), (Xap,Cc,YZbh),(Xap,Cc,YZbi),(Xap,Cc,YZbj),(Xap,Cc,YZbk),(Xap,Cc,YZbl),(Xap, Cc,YZbm),(Xap,Cc,YZbn),(Xap,Cc,YZbo),(Xap,Cc,YZbp),(Xap,Cc,YZbq),(Xap, Cc,YZbr),(Xap,Cc,YZbs),(Xap,Cc,YZbt),(Xap,Cc,YZbu),(Xap,Cc,YZbv),(Xap,Cc,YZbw), (Xap,Cc,YZbx),(Xap,Cc,YZby),(Xap,Cc,YZbz),(Xap,Cc,YZca),(Xap,Cc,YZcb),(Xap, Cc,YZcc),(Xap,Cc,YZcd),(Xap,Cc,YZce),(Xap,Cc,YZcf),(Xap,Cc,YZcg),(Xap,Cc, YZch),(Xap,Cc,YZci),(Xap,Cc,YZcj),(Xap,Cc,YZck),(Xap,Cc,YZcl),(Xap,Cc,YZcm), (Xap,Cc,YZcn),(Xap,Cc,YZco),(Xap,Cc,YZcp),(Xap,Cc,YZcq),(Xap,Cc,YZcr),(Xap, Cc,YZcs),(Xap,Cc,YZct),(Xap,Cc,YZcu),(Xap,Cc,YZcv),(Xap,Cc,YZcw),(Xap,Cc,YZcx), (Xap,Cc,YZcy),(Xap,Cc,YZcz),(Xap,Cc,YZda),(Xap,Cc,YZdb),(Xap,Cc,YZdc),(Xap, Cc,YZdd),(Xap,Cc,YZde),(Xap,Cc,YZdf),(Xap,Cc,YZdg),(Xap,Cc,YZdh),(Xap,Cc, YZdi),(Xap,Cc,YZdj),(Xap,Cc,YZdk),(Xap,Cc,YZdl),(Xap,Cc,YZdm),(Xap,Cc,YZdn), (Xap,Cc,YZdo),(Xap,Cc,YZdp),(Xap,Cc,YZdq),(Xap,Cc,YZdr),(Xap,Cc,YZds),(Xap, Cc,Yzdt),(Xap,Cc,YZdu),(Xap,Cc,YZdv),(Xap,Cc,YZdw),(Xap,Cc,YZdx),(Xap,Cc, YZdy),(Xap,Cc,YZdz),(Xap,Cc,YZea),(Xap,Cc,YZeb),(Xap,Cc,YZec),(Xap,Cc,YZed), (Xap,Cc,YZee),(Xap,Cc,YZef),(Xap,Cc,YZeg),(Xap,Cc,YZeh),(Xap,Cc,YZei),(Xap, Cc,YZej),(Xap,Cc,YZek),(Xap,Cc,YZel),(Xap,Cc,YZem),(Xap,Cc,YZen),(Xap,Cc,YZeo), (Xap,Cc,YZep),(Xap,Cc,YZeq),(Xap,Cc,YZer),(Xap,Cc,YZes),(Xap,Cc,YZet),(Xap, Cc,YZeu),(Xap,Cc,YZev),(Xap,Cc,YZew),(Xap,Cc,YZex),(Xap,Cc,YZey),(Xap,Cc, YZez),(Xap,Cc,YZfa),(Xap,Cc,YZfb),(Xap,Cc,YZfc),(Xap,Cc,YZfd),(Xap,Cc,YZfe),(Xap, Cc,YZff),(Xap,Cc,YZfg),(Xap,Cc,YZfh),(Xap,Cc,YZfi),(Xap,Cc,YZfj),(Xap,Cc, YZfk),(Xap,Cc,YZfl),(Xap,Cc,YZfm),(Xap,Cc,YZfn),(Xap,Cc,YZfo),(Xap,Cc,YZfp),(Xap, Cc,YZfq),(Xap,Cc,YZfr),(Xap,Cc,YZfs),(Xap,Cc,YZft),(Xap,Cc,YZfu),(Xap,Cc, YZfv),(Xap,Cc,YZfw),(Xap,Cc,YZfx),(Xap,Cc,YZfy),(Xap,Cc,YZfz),(Xap,Cc,YZga), (Xap,Cc,YZgb),(Xap,Cc,YZgc),(Xap,Cc,YZgd),(Xap,Cc,YZge),(Xap,Cc,YZgf),(Xap, Cc,YZgg),(Xap,Cc,YZgh),(Xap,Cc,YZgi),(Xap,Cc,YZgj),(Xap,Cc,YZgk),(Xap,Cc,YZgl), (Xap,Cc,YZgm),(Xap,Cc,YZgn),(Xap,Cc,YZgo),(Xap,Cc,YZgp),(Xap,Cc,YZgq),(Xap, Cc,YZgr),(Xap,Cc,YZgs),(Xap,Cc,YZgt),(Xap,Cd,YZa),(Xap,Cd,YZb),(Xap,Cd, YZc),(Xap,Cd,YZd),(Xap,Cd,YZe),(Xap,Cd,YZf),(Xap,Cd,YZg),(Xap,Cd,YZh),(Xap, Cd,YZi),(Xap,Cd,YZj),(Xap,Cd,YZk),(Xap,Cd,YZl),(Xap,Cd,YZm),(Xap,Cd,YZn),(Xap, Cd,YZo),(Xap,Cd,YZp),(Xap,Cd,YZq),(Xap,Cd,YZr),(Xap,Cd,YZs),(Xap,Cd,YZt), (Xap,Cd,YZu),(Xap,Cd,YZv),(Xap,Cd,YZw),(Xap,Cd,YZx),(Xap,Cd,YZy),(Xap,Cd, YZz),(Xap,Cd,YZaa),(Xap,Cd,YZab),(Xap,Cd,YZac),(Xap,Cd,YZad),(Xap,Cd,YZae), (Xap,Cd,YZaf),(Xap,Cd,YZag),(Xap,Cd,YZah),(Xap,Cd,YZai),(Xap,Cd,YZaj),(Xap,Cd, YZak),(Xap,Cd,YZal),(Xap,Cd,YZam),(Xap,Cd,YZan),(Xap,Cd,YZao),(Xap,Cd,YZap), (Xap,Cd,YZaq),(Xap,Cd,YZar),(Xap,Cd,YZas),(Xap,Cd,YZat),(Xap,Cd,YZau),(Xap, Cd,YZav),(Xap,Cd,YZaw),(Xap,Cd,YZax),(Xap,Cd,YZay),(Xap,Cd,YZaz),(Xap,Cd, YZba),(Xap,Cd,YZbb),(Xap,Cd,YZbc),(Xap,Cd,YZbd),(Xap,Cd,YZbe),(Xap,Cd,YZbf), (Xap,Cd,YZbg),(Xap,Cd,YZbh),(Xap,Cd,YZbi),(Xap,Cd,YZbj),(Xap,Cd,YZbk),(Xap, Cd,YZbl),(Xap,Cd,YZbm),(Xap,Cd,YZbn),(Xap,Cd,YZbo),(Xap,Cd,YZbp),(Xap,Cd, YZbq),(Xap,Cd,YZbr),(Xap,Cd,YZbs),(Xap,Cd,YZbt),(Xap,Cd,YZbu),(Xap,Cd,YZbv), (Xap,Cd,YZbw),(Xap,Cd,YZbx),(Xap,Cd,YZby),(Xap,Cd,YZbz),(Xap,Cd,YZca),(Xap, Cd,YZcb),(Xap,Cd,YZcc),(Xap,Cd,YZcd),(Xap,Cd,YZce),(Xap,Cd,YZcf),(Xap,Cd, YZcg),(Xap,Cd,YZch),(Xap,Cd,YZci),(Xap,Cd,YZcj),(Xap,Cd,YZck),(Xap,Cd,YZcl), (Xap,Cd,YZcm),(Xap,Cd,YZcn),(Xap,Cd,YZco),(Xap,Cd,YZcp),(Xap,Cd,YZcq),(Xap, Cd,YZcr),(Xap,Cd,YZcs),(Xap,Cd,YZct),(Xap,Cd,YZcu),(Xap,Cd,YZcv),(Xap,Cd, YZcw),(Xap,Cd,YZcx),(Xap,Cd,YZcy),(Xap,Cd,YZcz),(Xap,Cd,YZda),(Xap,Cd,YZdb), (Xap,Cd,YZdc),(Xap,Cd,YZdd),(Xap,Cd,YZde),(Xap,Cd,YZdf),(Xap,Cd,YZdg),(Xap, Cd,YZdh),(Xap,Cd,YZdi),(Xap,Cd,YZdj),(Xap,Cd,YZdk),(Xap,Cd,YZdl),(Xap,Cd, YZdm),(Xap,Cd,YZdn),(Xap,Cd,YZdo),(Xap,Cd,YZdp),(Xap,Cd,YZdq),(Xap,Cd,YZdr), (Xap,Cd,YZds),(Xap,Cd,Yzdt),(Xap,Cd,YZdu),(Xap,Cd,YZdv),(Xap,Cd,YZdw),(Xap, Cd,YZdx),(Xap,Cd,YZdy),(Xap,Cd,YZdz),(Xap,Cd,YZea),(Xap,Cd,YZeb),(Xap, Cd,YZec),(Xap,Cd,YZed),(Xap,Cd,YZee),(Xap,Cd,YZef),(Xap,Cd,YZeg),(Xap,Cd,YZeh), (Xap,Cd,YZei),(Xap,Cd,YZej),(Xap,Cd,YZek),(Xap,Cd,YZel),(Xap,Cd,YZem),(Xap, Cd,YZen),(Xap,Cd,YZeo),(Xap,Cd,YZep),(Xap,Cd,YZeq),(Xap,Cd,YZer),(Xap, Cd,YZes),(Xap,Cd,YZet),(Xap,Cd,YZeu),(Xap,Cd,YZev),(Xap,Cd,YZew),(Xap,Cd,YZex), (Xap,Cd,YZey),(Xap,Cd,YZez),(Xap,Cd,YZfa),(Xap,Cd,YZfb),(Xap,Cd,YZfc),(Xap, Cd,YZfd),(Xap,Cd,YZfe),(Xap,Cd,YZff),(Xap,Cd,YZfg),(Xap,Cd,YZfh),(Xap,Cd, YZfi),(Xap,Cd,YZfj),(Xap,Cd,YZfk),(Xap,Cd,YZfl),(Xap,Cd,YZfm),(Xap,Cd,YZfn), (Xap,Cd,YZfo),(Xap,Cd,YZfp),(Xap,Cd,YZfq),(Xap,Cd,YZfr),(Xap,Cd,YZfs),(Xap,Cd, YZft),(Xap,Cd,YZfu),(Xap,Cd,YZfv),(Xap,Cd,YZfw),(Xap,Cd,YZfx),(Xap,Cd,YZfy), (Xap,Cd,YZfz),(Xap,Cd,YZga),(Xap,Cd,YZgb),(Xap,Cd,YZgc),(Xap,Cd,YZgd),(Xap, Cd,YZge),(Xap,Cd,YZgf),(Xap,Cd,YZgg),(Xap,Cd,YZgh),(Xap,Cd,YZgi),(Xap,Cd, YZgj),(Xap,Cd,YZgk),(Xap,Cd,YZgl),(Xap,Cd,YZgm),(Xap,Cd,YZgn),(Xap,Cd,YZgo), (Xap,Cd,YZgp),(Xap,Cd,YZgq),(Xap,Cd,YZgr),(Xap,Cd,YZgs),(Xap,Cd,YZgt),(Xap, Ce,YZa),(Xap,Ce,YZb),(Xap,Ce,YZc),(Xap,Ce,YZd),(Xap,Ce,YZe),(Xap,Ce,YZf),(Xap, Ce,YZg),(Xap,Ce,YZh),(Xap,Ce,YZi),(Xap,Ce,YZj),(Xap,Ce,YZk),(Xap,Ce,YZl), (Xap,Ce,YZm),(Xap,Ce,YZn),(Xap,Ce,YZo),(Xap,Ce,YZp),(Xap,Ce,YZq),(Xap,Ce,YZr), (Xap,Ce,YZs),(Xap,Ce,YZt),(Xap,Ce,YZu),(Xap,Ce,YZv),(Xap,Ce,YZw),(Xap,Ce, YZx),(Xap,Ce,YZy),(Xap,Ce,YZz),(Xap,Ce,YZaa),(Xap,Ce,YZab),(Xap,Ce,YZac),(Xap, Ce,YZad),(Xap,Ce,YZae),(Xap,Ce,YZaf),(Xap,Ce,YZag),(Xap,Ce,YZah),(Xap,Ce, YZai),(Xap,Ce,YZaj),(Xap,Ce,YZak),(Xap,Ce,YZal),(Xap,Ce,YZam),(Xap,Ce,YZan), (Xap,Ce,YZao),(Xap,Ce,YZap),(Xap,Ce,YZaq),(Xap,Ce,YZar),(Xap,Ce,YZas),(Xap, Ce,YZat),(Xap,Ce,YZau),(Xap,Ce,YZav),(Xap,Ce,YZaw),(Xap,Ce,YZax),(Xap,Ce,YZay), (Xap,Ce,YZaz),(Xap,Ce,YZba),(Xap,Ce,YZbb),(Xap,Ce,YZbc),(Xap,Ce,YZbd),(Xap, Ce,YZbe),(Xap,Ce,YZbf),(Xap,Ce,YZbg),(Xap,Ce,YZbh),(Xap,Ce,YZbi),(Xap,Ce, YZbj),(Xap,Ce,YZbk),(Xap,Ce,YZbl),(Xap,Ce,YZbm),(Xap,Ce,YZbn),(Xap,Ce,YZbo), (Xap,Ce,YZbp),(Xap,Ce,YZbq),(Xap,Ce,YZbr),(Xap,Ce,YZbs),(Xap,Ce,YZbt),(Xap, Ce,YZbu),(Xap,Ce,YZbv),(Xap,Ce,YZbw),(Xap,Ce,YZbx),(Xap,Ce,YZby),(Xap,Ce, YZbz),(Xap,Ce,YZca),(Xap,Ce,YZcb),(Xap,Ce,YZcc),(Xap,Ce,YZcd),(Xap,Ce,YZce), (Xap,Ce,YZcf),(Xap,Ce,YZcg),(Xap,Ce,YZch),(Xap,Ce,YZci),(Xap,Ce,YZcj),(Xap,Ce, YZck),(Xap,Ce,YZcl),(Xap,Ce,YZcm),(Xap,Ce,YZcn),(Xap,Ce,YZco),(Xap,Ce,YZcp), (Xap,Ce,YZcq),(Xap,Ce,YZcr),(Xap,Ce,YZcs),(Xap,Ce,YZct),(Xap,Ce,YZcu),(Xap, Ce,YZcv),(Xap,Ce,YZcw),(Xap,Ce,YZcx),(Xap,Ce,YZcy),(Xap,Ce,YZcz),(Xap,Ce,YZda), (Xap,Ce,YZdb),(Xap,Ce,YZdc),(Xap,Ce,YZdd),(Xap,Ce,YZde),(Xap,Ce,YZdf),(Xap, Ce,YZdg),(Xap,Ce,YZdh),(Xap,Ce,YZdi),(Xap,Ce,YZdj),(Xap,Ce,YZdk),(Xap,Ce, YZdl),(Xap,Ce,YZdm),(Xap,Ce,YZdn),(Xap,Ce,YZdo),(Xap,Ce,YZdp),(Xap,Ce,YZdq), (Xap,Ce,YZdr),(Xap,Ce,YZds),(Xap,Ce,Yzdt),(Xap,Ce,YZdu),(Xap,Ce,YZdv),(Xap, Ce,YZdw),(Xap,Ce,YZdx),(Xap,Ce,YZdy),(Xap,Ce,YZdz),(Xap,Ce,YZea),(Xap,Ce, YZeb),(Xap,Ce,YZec),(Xap,Ce,YZed),(Xap,Ce,YZee),(Xap,Ce,YZef),(Xap,Ce,YZeg), (Xap,Ce,YZeh),(Xap,Ce,YZei),(Xap,Ce,YZej),(Xap,Ce,YZek),(Xap,Ce,YZel),(Xap,Ce, YZem),(Xap,Ce,YZen),(Xap,Ce,YZeo),(Xap,Ce,YZep),(Xap,Ce,YZeq),(Xap,Ce,YZer), (Xap,Ce,YZes),(Xap,Ce,YZet),(Xap,Ce,YZeu),(Xap,Ce,YZev),(Xap,Ce,YZew),(Xap, Ce,YZex),(Xap,Ce,YZey),(Xap,Ce,YZez),(Xap,Ce,YZfa),(Xap,Ce,YZfb),(Xap,Ce, YZfc),(Xap,Ce,YZfd),(Xap,Ce,YZfe),(Xap,Ce,YZff),(Xap,Ce,YZfg),(Xap,Ce,YZfh),(Xap, Ce,YZfi),(Xap,Ce,YZfj),(Xap,Ce,YZfk),(Xap,Ce,YZfl),(Xap,Ce,YZfm),(Xap,Ce, YZfn),(Xap,Ce,YZfo),(Xap,Ce,YZfp),(Xap,Ce,YZfq),(Xap,Ce,YZfr),(Xap,Ce,YZfs),(Xap, Ce,YZft),(Xap,Ce,YZfu),(Xap,Ce,YZfv),(Xap,Ce,YZfw),(Xap,Ce,YZfx),(Xap,Ce, YZfy),(Xap,Ce,YZfz),(Xap,Ce,YZga),(Xap,Ce,YZgb),(Xap,Ce,YZgc),(Xap,Ce,YZgd), (Xap,Ce,YZge),(Xap,Ce,YZgf),(Xap,Ce,YZgg),(Xap,Ce,YZgh),(Xap,Ce,YZgi),(Xap, Ce,YZgj),(Xap,Ce,YZgk),(Xap,Ce,YZgl),(Xap,Ce,YZgm),(Xap,Ce,YZgn),(Xap,Ce,YZgo), (Xap,Ce,YZgp),(Xap,Ce,YZgq),(Xap,Ce,YZgr),(Xap,Ce,YZgs),(Xap,Ce,YZgt),(Xap, Cf,YZa),(Xap,Cf,YZb),(Xap,Cf,YZc),(Xap,Cf,YZd),(Xap,Cf,YZe),(Xap,Cf,YZf), (Xap,Cf,YZg),(Xap,Cf,YZh),(Xap,Cf,YZi),(Xap,Cf,YZj),(Xap,Cf,YZk),(Xap,Cf,YZl), (Xap,Cf,YZm),(Xap,Cf,YZn),(Xap,Cf,YZo),(Xap,Cf,YZp),(Xap,Cf,YZq),(Xap,Cf,YZr), (Xap,Cf,YZs),(Xap,Cf,YZt),(Xap,Cf,YZu),(Xap,Cf,YZv),(Xap,Cf,YZw),(Xap,Cf,YZx), (Xap,Cf,YZy),(Xap,Cf,YZz),(Xap,Cf,YZaa),(Xap,Cf,YZab),(Xap,Cf,YZac),(Xap, Cf,YZad),(Xap,Cf,YZae),(Xap,Cf,YZaf),(Xap,Cf,YZag),(Xap,Cf,YZah),(Xap,Cf,YZai), (Xap,Cf,YZaj),(Xap,Cf,YZak),(Xap,Cf,YZal),(Xap,Cf,YZam),(Xap,Cf,YZan),(Xap, Cf,YZao),(Xap,Cf,YZap),(Xap,Cf,YZaq),(Xap,Cf,YZar),(Xap,Cf,YZas),(Xap,Cf,YZat), (Xap,Cf,YZau),(Xap,Cf,YZav),(Xap,Cf,YZaw),(Xap,Cf,YZax),(Xap,Cf,YZay),(Xap, Cf,YZaz),(Xap,Cf,YZba),(Xap,Cf,YZbb),(Xap,Cf,YZbc),(Xap,Cf,YZbd),(Xap,Cf,YZbe), (Xap,Cf,YZbf),(Xap,Cf,YZbg),(Xap,Cf,YZbh),(Xap,Cf,YZbi),(Xap,Cf,YZbj),(Xap, Cf,YZbk),(Xap,Cf,YZbl),(Xap,Cf,YZbm),(Xap,Cf,YZbn),(Xap,Cf,YZbo),(Xap,Cf,YZbp), (Xap,Cf,YZbq),(Xap,Cf,YZbr),(Xap,Cf,YZbs),(Xap,Cf,YZbt),(Xap,Cf,YZbu),(Xap, Cf,YZbv),(Xap,Cf,YZbw),(Xap,Cf,YZbx),(Xap,Cf,YZby),(Xap,Cf,YZbz),(Xap,Cf,YZca), (Xap,Cf,YZcb),(Xap,Cf,YZcc),(Xap,Cf,YZcd),(Xap,Cf,YZce),(Xap,Cf,YZcf),(Xap, Cf,YZcg),(Xap,Cf,YZch),(Xap,Cf,YZci),(Xap,Cf,YZcj),(Xap,Cf,YZck),(Xap,Cf,YZcl), (Xap,Cf,YZcm),(Xap,Cf,YZcn),(Xap,Cf,YZco),(Xap,Cf,YZcp),(Xap,Cf,YZcq),(Xap, Cf,YZcr),(Xap,Cf,YZcs),(Xap,Cf,YZct),(Xap,Cf,YZcu),(Xap,Cf,YZcv),(Xap,Cf, YZcw),(Xap,Cf,YZcx),(Xap,Cf,YZcy),(Xap,Cf,YZcz),(Xap,Cf,YZda),(Xap,Cf,YZdb), (Xap,Cf,YZdc),(Xap,Cf,YZdd),(Xap,Cf,YZde),(Xap,Cf,YZdf),(Xap,Cf,YZdg),(Xap,Cf, YZdh),(Xap,Cf,YZdi),(Xap,Cf,YZdj),(Xap,Cf,YZdk),(Xap,Cf,YZdl),(Xap,Cf,YZdm), (Xap,Cf,YZdn),(Xap,Cf,YZdo),(Xap,Cf,YZdp),(Xap,Cf,YZdq),(Xap,Cf,YZdr),(Xap, Cf,YZds),(Xap,Cf,Yzdt),(Xap,Cf,YZdu),(Xap,Cf,YZdv),(Xap,Cf,YZdw),(Xap,Cf,YZdx), (Xap,Cf,YZdy),(Xap,Cf,YZdz),(Xap,Cf,YZea),(Xap,Cf,YZeb),(Xap,Cf,YZec),(Xap, Cf,YZed),(Xap,Cf,YZee),(Xap,Cf,YZef),(Xap,Cf,YZeg),(Xap,Cf,YZeh),(Xap,Cf,YZei), (Xap,Cf,YZej),(Xap,Cf,YZek),(Xap,Cf,YZel),(Xap,Cf,YZem),(Xap,Cf,YZen),(Xap, Cf,YZeo),(Xap,Cf,YZep),(Xap,Cf,YZeq),(Xap,Cf,YZer),(Xap,Cf,YZes),(Xap,Cf,YZet), (Xap,Cf,YZeu),(Xap,Cf,YZev),(Xap,Cf,YZew),(Xap,Cf,YZex),(Xap,Cf,YZey),(Xap, Cf,YZez),(Xap,Cf,YZfa),(Xap,Cf,YZfb),(Xap,Cf,YZfc),(Xap,Cf,YZfd),(Xap,Cf,YZfe), (Xap,Cf,YZff),(Xap,Cf,YZfg),(Xap,Cf,YZfh),(Xap,Cf,YZfi),(Xap,Cf,YZfj),(Xap,Cf, YZfk),(Xap,Cf,YZfl),(Xap,Cf,YZfm),(Xap,Cf,YZfn),(Xap,Cf,YZfo),(Xap,Cf,YZfp),(Xap, Cf,YZfq),(Xap,Cf,YZfr),(Xap,Cf,YZfs),(Xap,Cf,YZft),(Xap,Cf,YZfu),(Xap,Cf,YZfv), (Xap,Cf,YZfw),(Xap,Cf,YZfx),(Xap,Cf,YZfy),(Xap,Cf,YZfz),(Xap,Cf,YZga),(Xap, Cf,YZgb),(Xap,Cf,YZgc),(Xap,Cf,YZgd),(Xap,Cf,YZge),(Xap,Cf,YZgf),(Xap,Cf,YZgg), (Xap,Cf,YZgh),(Xap,Cf,YZgi),(Xap,Cf,YZgj),(Xap,Cf,YZgk),(Xap,Cf,YZgl),(Xap, Cf,YZgm),(Xap,Cf,YZgn),(Xap,Cf,YZgo),(Xap,Cf,YZgp),(Xap,Cf,YZgq),(Xap,Cf, YZgr),(Xap,Cf,YZgs),(Xap,Cf,YZgt), (Xaq,Ca,YZa),(Xaq,Ca,YZb),(Xaq,Ca,YZc),(Xaq,Ca,YZd),(Xaq,Ca,YZe),(Xaq,Ca,YZf), (Xaq,Ca,YZg),(Xaq,Ca,YZh),(Xaq,Ca,YZi),(Xaq,Ca,YZj),(Xaq,Ca,YZk),(Xaq,Ca,YZl), (Xaq,Ca,YZm),(Xaq,Ca,YZn),(Xaq,Ca,YZo),(Xaq,Ca,YZp),(Xaq,Ca,YZq),(Xaq,Ca, YZr),(Xaq,Ca,YZs),(Xaq,Ca,YZt),(Xaq,Ca,YZu),(Xaq,Ca,YZv),(Xaq,Ca,YZw),(Xaq, Ca,YZx),(Xaq,Ca,YZy),(Xaq,Ca,YZz),(Xaq,Ca,YZaa),(Xaq,Ca,YZab),(Xaq,Ca,YZac), (Xaq,Ca,YZad),(Xaq,Ca,YZae),(Xaq,Ca,YZaf),(Xaq,Ca,YZag),(Xaq,Ca,YZah),(Xaq, Ca,YZai),(Xaq,Ca,YZaj),(Xaq,Ca,YZak),(Xaq,Ca,YZal),(Xaq,Ca,YZam),(Xaq,Ca,YZan), (Xaq,Ca,YZao),(Xaq,Ca,YZap),(Xaq,Ca,YZaq),(Xaq,Ca,YZar),(Xaq,Ca,YZas),(Xaq, Ca,YZat),(Xaq,Ca,YZau),(Xaq,Ca,YZav),(Xaq,Ca,YZaw),(Xaq,Ca,YZax),(Xaq,Ca, YZay),(Xaq,Ca,YZaz),(Xaq,Ca,YZba),(Xaq,Ca,YZbb),(Xaq,Ca,YZbc),(Xaq,Ca,YZbd), (Xaq,Ca,YZbe),(Xaq,Ca,YZbf),(Xaq,Ca,YZbg),(Xaq,Ca,YZbh),(Xaq,Ca,YZbi),(Xaq, Ca,YZbj),(Xaq,Ca,YZbk),(Xaq,Ca,YZbl),(Xaq,Ca,YZbm),(Xaq,Ca,YZbn),(Xaq,Ca,YZbo), (Xaq,Ca,YZbp),(Xaq,Ca,YZbq),(Xaq,Ca,YZbr),(Xaq,Ca,YZbs),(Xaq,Ca,YZbt),(Xaq, Ca,YZbu),(Xaq,Ca,YZbv),(Xaq,Ca,YZbw),(Xaq,Ca,YZbx),(Xaq,Ca,YZby),(Xaq, Ca,YZbz),(Xaq,Ca,YZca),(Xaq,Ca,YZcb),(Xaq,Ca,YZcc),(Xaq,Ca,YZcd),(Xaq,Ca,YZce), (Xaq,Ca,YZcf),(Xaq,Ca,YZcg),(Xaq,Ca,YZch),(Xaq,Ca,YZci),(Xaq,Ca,YZcj),(Xaq, Ca,YZck),(Xaq,Ca,YZcl),(Xaq,Ca,YZcm),(Xaq,Ca,YZcn),(Xaq,Ca,YZco),(Xaq,Ca, YZcp),(Xaq,Ca,YZcq),(Xaq,Ca,YZcr),(Xaq,Ca,YZcs),(Xaq,Ca,YZct),(Xaq,Ca,YZcu), (Xaq,Ca,YZcv),(Xaq,Ca,YZcw),(Xaq,Ca,YZcx),(Xaq,Ca,YZcy),(Xaq,Ca,YZcz),(Xaq, Ca,YZda),(Xaq,Ca,YZdb),(Xaq,Ca,YZdc),(Xaq,Ca,YZdd),(Xaq,Ca,YZde),(Xaq,Ca,YZdf), (Xaq,Ca,YZdg),(Xaq,Ca,YZdh),(Xaq,Ca,YZdi),(Xaq,Ca,YZdj),(Xaq,Ca,YZdk),(Xaq, Ca,YZdl),(Xaq,Ca,YZdm),(Xaq,Ca,YZdn),(Xaq,Ca,YZdo),(Xaq,Ca,YZdp),(Xaq, Ca,YZdq),(Xaq,Ca,YZdr),(Xaq,Ca,YZds),(Xaq,Ca,Yzdt),(Xaq,Ca,YZdu),(Xaq,Ca,YZdv), (Xaq,Ca,YZdw),(Xaq,Ca,YZdx),(Xaq,Ca,YZdy),(Xaq,Ca,YZdz),(Xaq,Ca,YZea),(Xaq, Ca,YZeb),(Xaq,Ca,YZec),(Xaq,Ca,YZed),(Xaq,Ca,YZee),(Xaq,Ca,YZef),(Xaq,Ca, YZeg),(Xaq,Ca,YZeh),(Xaq,Ca,YZei),(Xaq,Ca,YZej),(Xaq,Ca,YZek),(Xaq,Ca,YZel), (Xaq,Ca,YZem),(Xaq,Ca,YZen),(Xaq,Ca,YZeo),(Xaq,Ca,YZep),(Xaq,Ca,YZeq),(Xaq, Ca,YZer),(Xaq,Ca,YZes),(Xaq,Ca,YZet),(Xaq,Ca,YZeu),(Xaq,Ca,YZev),(Xaq,Ca,YZew), (Xaq,Ca,YZex),(Xaq,Ca,YZey),(Xaq,Ca,YZez),(Xaq,Ca,YZfa),(Xaq,Ca,YZfb),(Xaq, Ca,YZfc),(Xaq,Ca,YZfd),(Xaq,Ca,YZfe),(Xaq,Ca,YZff),(Xaq,Ca,YZfg),(Xaq,Ca,YZfh), (Xaq,Ca,YZfi),(Xaq,Ca,YZfj),(Xaq,Ca,YZfk),(Xaq,Ca,YZfl),(Xaq,Ca,YZfm),(Xaq, Ca,YZfn),(Xaq,Ca,YZfo),(Xaq,Ca,YZfp),(Xaq,Ca,YZfq),(Xaq,Ca,YZfr),(Xaq,Ca,YZfs), (Xaq,Ca,YZft),(Xaq,Ca,YZfu),(Xaq,Ca,YZfv),(Xaq,Ca,YZfw),(Xaq,Ca,YZfx),(Xaq, Ca,YZfy),(Xaq,Ca,YZfz),(Xaq,Ca,YZga),(Xaq,Ca,YZgb),(Xaq,Ca,YZgc),(Xaq,Ca, YZgd),(Xaq,Ca,YZge),(Xaq,Ca,YZgf),(Xaq,Ca,YZgg),(Xaq,Ca,YZgh),(Xaq,Ca,YZgi), (Xaq,Ca,YZgj),(Xaq,Ca,YZgk),(Xaq,Ca,YZgl),(Xaq,Ca,YZgm),(Xaq,Ca,YZgn),(Xaq, Ca,YZgo),(Xaq,Ca,YZgp),(Xaq,Ca,YZgq),(Xaq,Ca,YZgr),(Xaq,Ca,YZgs),(Xaq,Ca,YZgt), (Xaq,Cb,YZa),(Xaq,Cb,YZb),(Xaq,Cb,YZc),(Xaq,Cb,YZd),(Xaq,Cb,YZe),(Xaq, Cb,YZf),(Xaq,Cb,YZg),(Xaq,Cb,YZh),(Xaq,Cb,YZi),(Xaq,Cb,YZj),(Xaq,Cb,YZk),(Xaq, Cb,YZl),(Xaq,Cb,YZm),(Xaq,Cb,YZn),(Xaq,Cb,YZo),(Xaq,Cb,YZp),(Xaq,Cb,YZq), (Xaq,Cb,YZr),(Xaq,Cb,YZs),(Xaq,Cb,YZt),(Xaq,Cb,YZu),(Xaq,Cb,YZv),(Xaq,Cb,YZw), (Xaq,Cb,YZx),(Xaq,Cb,YZy),(Xaq,Cb,YZz),(Xaq,Cb,YZaa),(Xaq,Cb,YZab),(Xaq, Cb,YZac),(Xaq,Cb,YZad),(Xaq,Cb,YZae),(Xaq,Cb,YZaf),(Xaq,Cb,YZag),(Xaq,Cb, YZah),(Xaq,Cb,YZai),(Xaq,Cb,YZaj),(Xaq,Cb,YZak),(Xaq,Cb,YZal),(Xaq,Cb,YZam), (Xaq,Cb,YZan),(Xaq,Cb,YZao),(Xaq,Cb,YZap),(Xaq,Cb,YZaq),(Xaq,Cb,YZar),(Xaq, Cb,YZas),(Xaq,Cb,YZat),(Xaq,Cb,YZau),(Xaq,Cb,YZav),(Xaq,Cb,YZaw),(Xaq,Cb,YZax), (Xaq,Cb,YZay),(Xaq,Cb,YZaz),(Xaq,Cb,YZba),(Xaq,Cb,YZbb),(Xaq,Cb,YZbc), (Xaq,Cb,YZbd),(Xaq,Cb,YZbe),(Xaq,Cb,YZbf),(Xaq,Cb,YZbg),(Xaq,Cb,YZbh),(Xaq, Cb,YZbi),(Xaq,Cb,YZbj),(Xaq,Cb,YZbk),(Xaq,Cb,YZbl),(Xaq,Cb,YZbm),(Xaq,Cb,YZbn), (Xaq,Cb,YZbo),(Xaq,Cb,YZbp),(Xaq,Cb,YZbq),(Xaq,Cb,YZbr),(Xaq,Cb,YZbs), (Xaq,Cb,YZbt),(Xaq,Cb,YZbu),(Xaq,Cb,YZbv),(Xaq,Cb,YZbw),(Xaq,Cb,YZbx),(Xaq, Cb,YZby),(Xaq,Cb,YZbz),(Xaq,Cb,YZca),(Xaq,Cb,YZcb),(Xaq,Cb,YZcc),(Xaq,Cb,YZcd), (Xaq,Cb,YZce),(Xaq,Cb,YZcf),(Xaq,Cb,YZcg),(Xaq,Cb,YZch),(Xaq,Cb,YZci),(Xaq, Cb,YZcj),(Xaq,Cb,YZck),(Xaq,Cb,YZcl),(Xaq,Cb,YZcm),(Xaq,Cb,YZcn),(Xaq, Cb,YZco),(Xaq,Cb,YZcp),(Xaq,Cb,YZcq),(Xaq,Cb,YZcr),(Xaq,Cb,YZcs),(Xaq,Cb,YZct), (Xaq,Cb,YZcu),(Xaq,Cb,YZcv),(Xaq,Cb,YZcw),(Xaq,Cb,YZcx),(Xaq,Cb,YZcy), (Xaq,Cb,YZcz),(Xaq,Cb,YZda),(Xaq,Cb,YZdb),(Xaq,Cb,YZdc),(Xaq,Cb,YZdd),(Xaq, Cb,YZde),(Xaq,Cb,YZdf),(Xaq,Cb,YZdg),(Xaq,Cb,YZdh),(Xaq,Cb,YZdi),(Xaq,Cb,YZdj), (Xaq,Cb,YZdk),(Xaq,Cb,YZdl),(Xaq,Cb,YZdm),(Xaq,Cb,YZdn),(Xaq,Cb,YZdo), (Xaq,Cb,YZdp),(Xaq,Cb,YZdq),(Xaq,Cb,YZdr),(Xaq,Cb,YZds),(Xaq,Cb,Yzdt),(Xaq, Cb,YZdu),(Xaq,Cb,YZdv),(Xaq,Cb,YZdw),(Xaq,Cb,YZdx),(Xaq,Cb,YZdy),(Xaq,Cb, YZdz),(Xaq,Cb,YZea),(Xaq,Cb,YZeb),(Xaq,Cb,YZec),(Xaq,Cb,YZed),(Xaq,Cb,YZee), (Xaq,Cb,YZef),(Xaq,Cb,YZeg),(Xaq,Cb,YZeh),(Xaq,Cb,YZei),(Xaq,Cb,YZej),(Xaq, Cb,YZek),(Xaq,Cb,YZel),(Xaq,Cb,YZem),(Xaq,Cb,YZen),(Xaq,Cb,YZeo),(Xaq,Cb,YZep), (Xaq,Cb,YZeq),(Xaq,Cb,YZer),(Xaq,Cb,YZes),(Xaq,Cb,YZet),(Xaq,Cb,YZeu),(Xaq, Cb,YZev),(Xaq,Cb,YZew),(Xaq,Cb,YZex),(Xaq,Cb,YZey),(Xaq,Cb,YZez),(Xaq, Cb,YZfa),(Xaq,Cb,YZfb),(Xaq,Cb,YZfc),(Xaq,Cb,YZfd),(Xaq,Cb,YZfe),(Xaq,Cb,YZff), (Xaq,Cb,YZfg),(Xaq,Cb,YZfh),(Xaq,Cb,YZfi),(Xaq,Cb,YZfj),(Xaq,Cb,YZfk),(Xaq, Cb,YZfl),(Xaq,Cb,YZfm),(Xaq,Cb,YZfn),(Xaq,Cb,YZfo),(Xaq,Cb,YZfp),(Xaq,Cb,YZfq), (Xaq,Cb,YZfr),(Xaq,Cb,YZfs),(Xaq,Cb,YZft),(Xaq,Cb,YZfu),(Xaq,Cb,YZfv),(Xaq, Cb,YZfw),(Xaq,Cb,YZfx),(Xaq,Cb,YZfy),(Xaq,Cb,YZfz),(Xaq,Cb,YZga),(Xaq,Cb,YZgb), (Xaq,Cb,YZgc),(Xaq,Cb,YZgd),(Xaq,Cb,YZge),(Xaq,Cb,YZgf),(Xaq,Cb,YZgg), (Xaq,Cb,YZgh),(Xaq,Cb,YZgi),(Xaq,Cb,YZgj),(Xaq,Cb,YZgk),(Xaq,Cb,YZgl),(Xaq, Cb,YZgm),(Xaq,Cb,YZgn),(Xaq,Cb,YZgo),(Xaq,Cb,YZgp),(Xaq,Cb,YZgq),(Xaq,Cb, YZgr),(Xaq,Cb,YZgs),(Xaq,Cb,YZgt),(Xaq,Cc,YZa),(Xaq,Cc,YZb),(Xaq,Cc,YZc),(Xaq, Cc,YZd),(Xaq,Cc,YZe),(Xaq,Cc,YZf),(Xaq,Cc,YZg),(Xaq,Cc,YZh),(Xaq,Cc,YZi),(Xaq, Cc,YZj),(Xaq,Cc,YZk),(Xaq,Cc,YZl),(Xaq,Cc,YZm),(Xaq,Cc,YZn),(Xaq,Cc,YZo), (Xaq,Cc,YZp),(Xaq,Cc,YZq),(Xaq,Cc,YZr),(Xaq,Cc,YZs),(Xaq,Cc,YZt),(Xaq,Cc, YZu),(Xaq,Cc,YZv),(Xaq,Cc,YZw),(Xaq,Cc,YZx),(Xaq,Cc,YZy),(Xaq,Cc,YZz),(Xaq, Cc,YZaa),(Xaq,Cc,YZab),(Xaq,Cc,YZac),(Xaq,Cc,YZad),(Xaq,Cc,YZae),(Xaq,Cc,YZaf), (Xaq,Cc,YZag),(Xaq,Cc,YZah),(Xaq,Cc,YZai),(Xaq,Cc,YZaj),(Xaq,Cc,YZak),(Xaq, Cc,YZal),(Xaq,Cc,YZam),(Xaq,Cc,YZan),(Xaq,Cc,YZao),(Xaq,Cc,YZap),(Xaq,Cc, YZaq),(Xaq,Cc,YZar),(Xaq,Cc,YZas),(Xaq,Cc,YZat),(Xaq,Cc,YZau),(Xaq,Cc,YZav), (Xaq,Cc,YZaw),(Xaq,Cc,YZax),(Xaq,Cc,YZay),(Xaq,Cc,YZaz),(Xaq,Cc,YZba),(Xaq, Cc,YZbb),(Xaq,Cc,YZbc),(Xaq,Cc,YZbd),(Xaq,Cc,YZbe),(Xaq,Cc,YZbf),(Xaq,Cc,YZbg), (Xaq,Cc,YZbh),(Xaq,Cc,YZbi),(Xaq,Cc,YZbj),(Xaq,Cc,YZbk),(Xaq,Cc,YZbl),(Xaq, Cc,YZbm),(Xaq,Cc,YZbn),(Xaq,Cc,YZbo),(Xaq,Cc,YZbp),(Xaq,Cc,YZbq),(Xaq, Cc,YZbr),(Xaq,Cc,YZbs),(Xaq,Cc,YZbt),(Xaq,Cc,YZbu),(Xaq,Cc,YZbv),(Xaq,Cc,YZbw), (Xaq,Cc,YZbx),(Xaq,Cc,YZby),(Xaq,Cc,YZbz),(Xaq,Cc,YZca),(Xaq,Cc,YZcb),(Xaq, Cc,YZcc),(Xaq,Cc,YZcd),(Xaq,Cc,YZce),(Xaq,Cc,YZcf),(Xaq,Cc,YZcg),(Xaq,Cc, YZch),(Xaq,Cc,YZci),(Xaq,Cc,YZcj),(Xaq,Cc,YZck),(Xaq,Cc,YZcl),(Xaq,Cc,YZcm), (Xaq,Cc,YZcn),(Xaq,Cc,YZco),(Xaq,Cc,YZcp),(Xaq,Cc,YZcq),(Xaq,Cc,YZcr),(Xaq, Cc,YZcs),(Xaq,Cc,YZct),(Xaq,Cc,YZcu),(Xaq,Cc,YZcv),(Xaq,Cc,YZcw),(Xaq,Cc,YZcx), (Xaq,Cc,YZcy),(Xaq,Cc,YZcz),(Xaq,Cc,YZda),(Xaq,Cc,YZdb),(Xaq,Cc,YZdc),(Xaq, Cc,YZdd),(Xaq,Cc,YZde),(Xaq,Cc,YZdf),(Xaq,Cc,YZdg),(Xaq,Cc,YZdh),(Xaq,Cc, YZdi),(Xaq,Cc,YZdj),(Xaq,Cc,YZdk),(Xaq,Cc,YZdl),(Xaq,Cc,YZdm),(Xaq,Cc,YZdn), (Xaq,Cc,YZdo),(Xaq,Cc,YZdp),(Xaq,Cc,YZdq),(Xaq,Cc,YZdr),(Xaq,Cc,YZds),(Xaq, Cc,Yzdt),(Xaq,Cc,YZdu),(Xaq,Cc,YZdv),(Xaq,Cc,YZdw),(Xaq,Cc,YZdx),(Xaq,Cc, YZdy),(Xaq,Cc,YZdz),(Xaq,Cc,YZea),(Xaq,Cc,YZeb),(Xaq,Cc,YZec),(Xaq,Cc,YZed), (Xaq,Cc,YZee),(Xaq,Cc,YZef),(Xaq,Cc,YZeg),(Xaq,Cc,YZeh),(Xaq,Cc,YZei),(Xaq, Cc,YZej),(Xaq,Cc,YZek),(Xaq,Cc,YZel),(Xaq,Cc,YZem),(Xaq,Cc,YZen),(Xaq,Cc,YZeo), (Xaq,Cc,YZep),(Xaq,Cc,YZeq),(Xaq,Cc,YZer),(Xaq,Cc,YZes),(Xaq,Cc,YZet),(Xaq, Cc,YZeu),(Xaq,Cc,YZev),(Xaq,Cc,YZew),(Xaq,Cc,YZex),(Xaq,Cc,YZey),(Xaq,Cc, YZez),(Xaq,Cc,YZfa),(Xaq,Cc,YZfb),(Xaq,Cc,YZfe),(Xaq,Cc,YZfd),(Xaq,Cc,YZfe),(Xaq, Cc,YZff),(Xaq,Cc,YZfg),(Xaq,Cc,YZfh),(Xaq,Cc,YZfi),(Xaq,Cc,YZfj),(Xaq,Cc, YZfk),(Xaq,Cc,YZfl),(Xaq,Cc,YZfm),(Xaq,Cc,YZfn),(Xaq,Cc,YZfo),(Xaq,Cc,YZfp),(Xaq, Cc,YZfq),(Xaq,Cc,YZfr),(Xaq,Cc,YZfs),(Xaq,Cc,YZft),(Xaq,Cc,YZfu),(Xaq,Cc, YZfv),(Xaq,Cc,YZfw),(Xaq,Cc,YZfx),(Xaq,Cc,YZfy),(Xaq,Cc,YZfz),(Xaq,Cc,YZga), (Xaq,Cc,YZgb),(Xaq,Cc,YZgc),(Xaq,Cc,YZgd),(Xaq,Cc,YZge),(Xaq,Cc,YZgf),(Xaq, Cc,YZgg),(Xaq,Cc,YZgh),(Xaq,Cc,YZgi),(Xaq,Cc,YZgj),(Xaq,Cc,YZgk),(Xaq,Cc,YZgl), (Xaq,Cc,YZgm),(Xaq,Cc,YZgn),(Xaq,Cc,YZgo),(Xaq,Cc,YZgp),(Xaq,Cc,YZgq),(Xaq, Cc,YZgr),(Xaq,Cc,YZgs),(Xaq,Cc,YZgt),(Xaq,Cd,YZa),(Xaq,Cd,YZb),(Xaq,Cd, YZc),(Xaq,Cd,YZd),(Xaq,Cd,YZe),(Xaq,Cd,YZf),(Xaq,Cd,YZg),(Xaq,Cd,YZh),(Xaq, Cd,YZi),(Xaq,Cd,YZj),(Xaq,Cd,YZk),(Xaq,Cd,YZl),(Xaq,Cd,YZm),(Xaq,Cd,YZn),(Xaq, Cd,YZo),(Xaq,Cd,YZp),(Xaq,Cd,YZq),(Xaq,Cd,YZr),(Xaq,Cd,YZs),(Xaq,Cd,YZt), (Xaq,Cd,YZu),(Xaq,Cd,YZv),(Xaq,Cd,YZw),(Xaq,Cd,YZx),(Xaq,Cd,YZy),(Xaq,Cd, YZz),(Xaq,Cd,YZaa),(Xaq,Cd,YZab),(Xaq,Cd,YZac),(Xaq,Cd,YZad),(Xaq,Cd,YZae), (Xaq,Cd,YZaf),(Xaq,Cd,YZag),(Xaq,Cd,YZah),(Xaq,Cd,YZai),(Xaq,Cd,YZaj),(Xaq,Cd, YZak),(Xaq,Cd,YZal),(Xaq,Cd,YZam),(Xaq,Cd,YZan),(Xaq,Cd,YZao),(Xaq,Cd,YZap), (Xaq,Cd,YZaq),(Xaq,Cd,YZar),(Xaq,Cd,YZas),(Xaq,Cd,YZat),(Xaq,Cd,YZau),(Xaq, Cd,YZav),(Xaq,Cd,YZaw),(Xaq,Cd,YZax),(Xaq,Cd,YZay),(Xaq,Cd,YZaz),(Xaq,Cd, YZba),(Xaq,Cd,YZbb),(Xaq,Cd,YZbc),(Xaq,Cd,YZbd),(Xaq,Cd,YZbe),(Xaq,Cd,YZbf), (Xaq,Cd,YZbg),(Xaq,Cd,YZbh),(Xaq,Cd,YZbi),(Xaq,Cd,YZbj),(Xaq,Cd,YZbk),(Xaq, Cd,YZbl),(Xaq,Cd,YZbm),(Xaq,Cd,YZbn),(Xaq,Cd,YZbo),(Xaq,Cd,YZbp),(Xaq,Cd, YZbq),(Xaq,Cd,YZbr),(Xaq,Cd,YZbs),(Xaq,Cd,YZbt),(Xaq,Cd,YZbu),(Xaq,Cd,YZbv), (Xaq,Cd,YZbw),(Xaq,Cd,YZbx),(Xaq,Cd,YZby),(Xaq,Cd,YZbz),(Xaq,Cd,YZca),(Xaq, Cd,YZcb),(Xaq,Cd,YZcc),(Xaq,Cd,YZcd),(Xaq,Cd,YZce),(Xaq,Cd,YZcf),(Xaq,Cd, YZcg),(Xaq,Cd,YZch),(Xaq,Cd,YZci),(Xaq,Cd,YZcj),(Xaq,Cd,YZck),(Xaq,Cd,YZcl), (Xaq,Cd,YZcm),(Xaq,Cd,YZcn),(Xaq,Cd,YZco),(Xaq,Cd,YZcp),(Xaq,Cd,YZcq),(Xaq, Cd,YZcr),(Xaq,Cd,YZcs),(Xaq,Cd,YZct),(Xaq,Cd,YZcu),(Xaq,Cd,YZcv),(Xaq,Cd, YZcw),(Xaq,Cd,YZcx),(Xaq,Cd,YZcy),(Xaq,Cd,YZcz),(Xaq,Cd,YZda),(Xaq,Cd,YZdb), (Xaq,Cd,YZdc),(Xaq,Cd,YZdd),(Xaq,Cd,YZde),(Xaq,Cd,YZdf),(Xaq,Cd,YZdg),(Xaq, Cd,YZdh),(Xaq,Cd,YZdi),(Xaq,Cd,YZdj),(Xaq,Cd,YZdk),(Xaq,Cd,YZdl),(Xaq,Cd, YZdm),(Xaq,Cd,YZdn),(Xaq,Cd,YZdo),(Xaq,Cd,YZdp),(Xaq,Cd,YZdq),(Xaq,Cd,YZdr), (Xaq,Cd,YZds),(Xaq,Cd,Yzdt),(Xaq,Cd,YZdu),(Xaq,Cd,YZdv),(Xaq,Cd,YZdw),(Xaq, Cd,YZdx),(Xaq,Cd,YZdy),(Xaq,Cd,YZdz),(Xaq,Cd,YZea),(Xaq,Cd,YZeb),(Xaq, Cd,YZec),(Xaq,Cd,YZed),(Xaq,Cd,YZee),(Xaq,Cd,YZef),(Xaq,Cd,YZeg),(Xaq,Cd,YZeh), (Xaq,Cd,YZei),(Xaq,Cd,YZej),(Xaq,Cd,YZek),(Xaq,Cd,YZel),(Xaq,Cd,YZem),(Xaq, Cd,YZen),(Xaq,Cd,YZeo),(Xaq,Cd,YZep),(Xaq,Cd,YZeq),(Xaq,Cd,YZer),(Xaq, Cd,YZes),(Xaq,Cd,YZet),(Xaq,Cd,YZeu),(Xaq,Cd,YZev),(Xaq,Cd,YZew),(Xaq,Cd,YZex), (Xaq,Cd,YZey),(Xaq,Cd,YZez),(Xaq,Cd,YZfa),(Xaq,Cd,YZfb),(Xaq,Cd,YZfc),(Xaq, Cd,YZfd),(Xaq,Cd,YZfe),(Xaq,Cd,YZff),(Xaq,Cd,YZfg),(Xaq,Cd,YZfh),(Xaq,Cd, YZfi),(Xaq,Cd,YZfj),(Xaq,Cd,YZfk),(Xaq,Cd,YZfl),(Xaq,Cd,YZfm),(Xaq,Cd,YZfn), (Xaq,Cd,YZfo),(Xaq,Cd,YZfp),(Xaq,Cd,YZfq),(Xaq,Cd,YZfr),(Xaq,Cd,YZfs),(Xaq,Cd, YZft),(Xaq,Cd,YZfu),(Xaq,Cd,YZfv),(Xaq,Cd,YZfw),(Xaq,Cd,YZfx),(Xaq,Cd,YZfy), (Xaq,Cd,YZfz),(Xaq,Cd,YZga),(Xaq,Cd,YZgb),(Xaq,Cd,YZgc),(Xaq,Cd,YZgd),(Xaq, Cd,YZge),(Xaq,Cd,YZgf),(Xaq,Cd,YZgg),(Xaq,Cd,YZgh),(Xaq,Cd,YZgi),(Xaq,Cd, YZgj),(Xaq,Cd,YZgk),(Xaq,Cd,YZgl),(Xaq,Cd,YZgm),(Xaq,Cd,YZgn),(Xaq,Cd,YZgo), (Xaq,Cd,YZgp),(Xaq,Cd,YZgq),(Xaq,Cd,YZgr),(Xaq,Cd,YZgs),(Xaq,Cd,YZgt),(Xaq, Ce,YZa),(Xaq,Ce,YZb),(Xaq,Ce,YZc),(Xaq,Ce,YZd),(Xaq,Ce,YZe),(Xaq,Ce,YZf),(Xaq, Ce,YZg),(Xaq,Ce,YZh),(Xaq,Ce,YZi),(Xaq,Ce,YZj),(Xaq,Ce,YZk),(Xaq,Ce,YZl), (Xaq,Ce,YZm),(Xaq,Ce,YZn),(Xaq,Ce,YZo),(Xaq,Ce,YZp),(Xaq,Ce,YZq),(Xaq,Ce,YZr), (Xaq,Ce,YZs),(Xaq,Ce,YZt),(Xaq,Ce,YZu),(Xaq,Ce,YZv),(Xaq,Ce,YZw),(Xaq,Ce, YZx),(Xaq,Ce,YZy),(Xaq,Ce,YZz),(Xaq,Ce,YZaa),(Xaq,Ce,YZab),(Xaq,Ce,YZac),(Xaq, Ce,YZad),(Xaq,Ce,YZae),(Xaq,Ce,YZaf),(Xaq,Ce,YZag),(Xaq,Ce,YZah),(Xaq,Ce, YZai),(Xaq,Ce,YZaj),(Xaq,Ce,YZak),(Xaq,Ce,YZal),(Xaq,Ce,YZam),(Xaq,Ce,YZan), (Xaq,Ce,YZao),(Xaq,Ce,YZap),(Xaq,Ce,YZaq),(Xaq,Ce,YZar),(Xaq,Ce,YZas),(Xaq, Ce,YZat),(Xaq,Ce,YZau),(Xaq,Ce,YZav),(Xaq,Ce,YZaw),(Xaq,Ce,YZax),(Xaq,Ce,YZay), (Xaq,Ce,YZaz),(Xaq,Ce,YZba),(Xaq,Ce,YZbb),(Xaq,Ce,YZbc),(Xaq,Ce,YZbd),(Xaq, Ce,YZbe),(Xaq,Ce,YZbf),(Xaq,Ce,YZbg),(Xaq,Ce,YZbh),(Xaq,Ce,YZbi),(Xaq,Ce, YZbj),(Xaq,Ce,YZbk),(Xaq,Ce,YZbl),(Xaq,Ce,YZbm),(Xaq,Ce,YZbn),(Xaq,Ce,YZbo), (Xaq,Ce,YZbp),(Xaq,Ce,YZbq),(Xaq,Ce,YZbr),(Xaq,Ce,YZbs),(Xaq,Ce,YZbt),(Xaq, Ce,YZbu),(Xaq,Ce,YZbv),(Xaq,Ce,YZbw),(Xaq,Ce,YZbx),(Xaq,Ce,YZby),(Xaq,Ce, YZbz),(Xaq,Ce,YZca),(Xaq,Ce,YZcb),(Xaq,Ce,YZcc),(Xaq,Ce,YZcd),(Xaq,Ce,YZce), (Xaq,Ce,YZcf),(Xaq,Ce,YZcg),(Xaq,Ce,YZch),(Xaq,Ce,YZci),(Xaq,Ce,YZcj),(Xaq,Ce, YZck),(Xaq,Ce,YZcl),(Xaq,Ce,YZcm),(Xaq,Ce,YZcn),(Xaq,Ce,YZco),(Xaq,Ce,YZcp), (Xaq,Ce,YZcq),(Xaq,Ce,YZcr),(Xaq,Ce,YZcs),(Xaq,Ce,YZct),(Xaq,Ce,YZcu),(Xaq, Ce,YZcv),(Xaq,Ce,YZcw),(Xaq,Ce,YZcx),(Xaq,Ce,YZcy),(Xaq,Ce,YZcz),(Xaq,Ce,YZda), (Xaq,Ce,YZdb),(Xaq,Ce,YZdc),(Xaq,Ce,YZdd),(Xaq,Ce,YZde),(Xaq,Ce,YZdf),(Xaq, Ce,YZdg),(Xaq,Ce,YZdh),(Xaq,Ce,YZdi),(Xaq,Ce,YZdj),(Xaq,Ce,YZdk),(Xaq,Ce, YZdl),(Xaq,Ce,YZdm),(Xaq,Ce,YZdn),(Xaq,Ce,YZdo),(Xaq,Ce,YZdp),(Xaq,Ce,YZdq), (Xaq,Ce,YZdr),(Xaq,Ce,YZds),(Xaq,Ce,Yzdt),(Xaq,Ce,YZdu),(Xaq,Ce,YZdv),(Xaq, Ce,YZdw),(Xaq,Ce,YZdx),(Xaq,Ce,YZdy),(Xaq,Ce,YZdz),(Xaq,Ce,YZea),(Xaq,Ce, YZeb),(Xaq,Ce,YZec),(Xaq,Ce,YZed),(Xaq,Ce,YZee),(Xaq,Ce,YZef),(Xaq,Ce,YZeg), (Xaq,Ce,YZeh),(Xaq,Ce,YZei),(Xaq,Ce,YZej),(Xaq,Ce,YZek),(Xaq,Ce,YZel),(Xaq,Ce, YZem),(Xaq,Ce,YZen),(Xaq,Ce,YZeo),(Xaq,Ce,YZep),(Xaq,Ce,YZeq),(Xaq,Ce,YZer), (Xaq,Ce,YZes),(Xaq,Ce,YZet),(Xaq,Ce,YZeu),(Xaq,Ce,YZev),(Xaq,Ce,YZew),(Xaq, Ce,YZex),(Xaq,Ce,YZey),(Xaq,Ce,YZez),(Xaq,Ce,YZfa),(Xaq,Ce,YZfb),(Xaq,Ce, YZfc),(Xaq,Ce,YZfd),(Xaq,Ce,YZfe),(Xaq,Ce,YZff),(Xaq,Ce,YZfg),(Xaq,Ce,YZfh),(Xaq, Ce,YZfi),(Xaq,Ce,YZfj),(Xaq,Ce,YZfk),(Xaq,Ce,YZfl),(Xaq,Ce,YZfm),(Xaq,Ce, YZfn),(Xaq,Ce,YZfo),(Xaq,Ce,YZfp),(Xaq,Ce,YZfq),(Xaq,Ce,YZfr),(Xaq,Ce,YZfs),(Xaq, Ce,YZft),(Xaq,Ce,YZfu),(Xaq,Ce,YZfv),(Xaq,Ce,YZfw),(Xaq,Ce,YZfx),(Xaq,Ce, YZfy),(Xaq,Ce,YZfz),(Xaq,Ce,YZga),(Xaq,Ce,YZgb),(Xaq,Ce,YZgc),(Xaq,Ce,YZgd), (Xaq,Ce,YZge),(Xaq,Ce,YZgf),(Xaq,Ce,YZgg),(Xaq,Ce,YZgh),(Xaq,Ce,YZgi),(Xaq, Ce,YZgj),(Xaq,Ce,YZgk),(Xaq,Ce,YZgl),(Xaq,Ce,YZgm),(Xaq,Ce,YZgn),(Xaq,Ce,YZgo), (Xaq,Ce,YZgp),(Xaq,Ce,YZgq),(Xaq,Ce,YZgr),(Xaq,Ce,YZgs),(Xaq,Ce,YZgt),(Xaq, Cf,YZa),(Xaq,Cf,YZb),(Xaq,Cf,YZc),(Xaq,Cf,YZd),(Xaq,Cf,YZe),(Xaq,Cf,YZf), (Xaq,Cf,YZg),(Xaq,Cf,YZh),(Xaq,Cf,YZi),(Xaq,Cf,YZj),(Xaq,Cf,YZk),(Xaq,Cf,YZl), (Xaq,Cf,YZm),(Xaq,Cf,YZn),(Xaq,Cf,YZo),(Xaq,Cf,YZp),(Xaq,Cf,YZq),(Xaq,Cf,YZr), (Xaq,Cf,YZs),(Xaq,Cf,YZt),(Xaq,Cf,YZu),(Xaq,Cf,YZv),(Xaq,Cf,YZw),(Xaq,Cf,YZx), (Xaq,Cf,YZy),(Xaq,Cf,YZz),(Xaq,Cf,YZaa),(Xaq,Cf,YZab),(Xaq,Cf,YZac),(Xaq, Cf,YZad),(Xaq,Cf,YZae),(Xaq,Cf,YZaf),(Xaq,Cf,YZag),(Xaq,Cf,YZah),(Xaq,Cf,YZai), (Xaq,Cf,YZaj),(Xaq,Cf,YZak),(Xaq,Cf,YZal),(Xaq,Cf,YZam),(Xaq,Cf,YZan),(Xaq, Cf,YZao),(Xaq,Cf,YZap),(Xaq,Cf,YZaq),(Xaq,Cf,YZar),(Xaq,Cf,YZas),(Xaq,Cf,YZat), (Xaq,Cf,YZau),(Xaq,Cf,YZav),(Xaq,Cf,YZaw),(Xaq,Cf,YZax),(Xaq,Cf,YZay),(Xaq, Cf,YZaz),(Xaq,Cf,YZba),(Xaq,Cf,YZbb),(Xaq,Cf,YZbc),(Xaq,Cf,YZbd),(Xaq,Cf,YZbe), (Xaq,Cf,YZbf),(Xaq,Cf,YZbg),(Xaq,Cf,YZbh),(Xaq,Cf,YZbi),(Xaq,Cf,YZbj),(Xaq, Cf,YZbk),(Xaq,Cf,YZbl),(Xaq,Cf,YZbm),(Xaq,Cf,YZbn),(Xaq,Cf,YZbo),(Xaq,Cf,YZbp), (Xaq,Cf,YZbq),(Xaq,Cf,YZbr),(Xaq,Cf,YZbs),(Xaq,Cf,YZbt),(Xaq,Cf,YZbu),(Xaq, Cf,YZbv),(Xaq,Cf,YZbw),(Xaq,Cf,YZbx),(Xaq,Cf,YZby),(Xaq,Cf,YZbz),(Xaq,Cf,YZca), (Xaq,Cf,YZcb),(Xaq,Cf,YZcc),(Xaq,Cf,YZcd),(Xaq,Cf,YZce),(Xaq,Cf,YZcf),(Xaq, Cf,YZcg),(Xaq,Cf,YZch),(Xaq,Cf,YZci),(Xaq,Cf,YZcj),(Xaq,Cf,YZck),(Xaq,Cf,YZcl), (Xaq,Cf,YZcm),(Xaq,Cf,YZcn),(Xaq,Cf,YZco),(Xaq,Cf,YZcp),(Xaq,Cf,YZcq),(Xaq, Cf,YZcr),(Xaq,Cf,YZcs),(Xaq,Cf,YZct),(Xaq,Cf,YZcu),(Xaq,Cf,YZcv),(Xaq,Cf, YZcw),(Xaq,Cf,YZcx),(Xaq,Cf,YZcy),(Xaq,Cf,YZcz),(Xaq,Cf,YZda),(Xaq,Cf,YZdb), (Xaq,Cf,YZdc),(Xaq,Cf,YZdd),(Xaq,Cf,YZde),(Xaq,Cf,YZdf),(Xaq,Cf,YZdg),(Xaq,Cf, YZdh),(Xaq,Cf,YZdi),(Xaq,Cf,YZdj),(Xaq,Cf,YZdk),(Xaq,Cf,YZdl),(Xaq,Cf,YZdm), (Xaq,Cf,YZdn),(Xaq,Cf,YZdo),(Xaq,Cf,YZdp),(Xaq,Cf,YZdq),(Xaq,Cf,YZdr),(Xaq, Cf,YZds),(Xaq,Cf,Yzdt),(Xaq,Cf,YZdu),(Xaq,Cf,YZdv),(Xaq,Cf,YZdw),(Xaq,Cf,YZdx), (Xaq,Cf,YZdy),(Xaq,Cf,YZdz),(Xaq,Cf,YZea),(Xaq,Cf,YZeb),(Xaq,Cf,YZec),(Xaq, Cf,YZed),(Xaq,Cf,YZee),(Xaq,Cf,YZef),(Xaq,Cf,YZeg),(Xaq,Cf,YZeh),(Xaq,Cf,YZei), (Xaq,Cf,YZej),(Xaq,Cf,YZek),(Xaq,Cf,YZel),(Xaq,Cf,YZem),(Xaq,Cf,YZen),(Xaq, Cf,YZeo),(Xaq,Cf,YZep),(Xaq,Cf,YZeq),(Xaq,Cf,YZer),(Xaq,Cf,YZes),(Xaq,Cf,YZet), (Xaq,Cf,YZeu),(Xaq,Cf,YZev),(Xaq,Cf,YZew),(Xaq,Cf,YZex),(Xaq,Cf,YZey),(Xaq, Cf,YZez),(Xaq,Cf,YZfa),(Xaq,Cf,YZfb),(Xaq,Cf,YZfc),(Xaq,Cf,YZfd),(Xaq,Cf,YZfe), (Xaq,Cf,YZff),(Xaq,Cf,YZfg),(Xaq,Cf,YZfh),(Xaq,Cf,YZfi),(Xaq,Cf,YZfj),(Xaq,Cf, YZfk),(Xaq,Cf,YZfl),(Xaq,Cf,YZfm),(Xaq,Cf,YZfn),(Xaq,Cf,YZfo),(Xaq,Cf,YZfp),(Xaq, Cf,YZfq),(Xaq,Cf,YZfr),(Xaq,Cf,YZfs),(Xaq,Cf,YZft),(Xaq,Cf,YZfu),(Xaq,Cf,YZfv), (Xaq,Cf,YZfw),(Xaq,Cf,YZfx),(Xaq,Cf,YZfy),(Xaq,Cf,YZfz),(Xaq,Cf,YZga),(Xaq, Cf,YZgb),(Xaq,Cf,YZgc),(Xaq,Cf,YZgd),(Xaq,Cf,YZge),(Xaq,Cf,YZgf),(Xaq,Cf,YZgg), (Xaq,Cf,YZgh),(Xaq,Cf,YZgi),(Xaq,Cf,YZgj),(Xaq,Cf,YZgk),(Xaq,Cf,YZgl),(Xaq, Cf,YZgm),(Xaq,Cf,YZgn),(Xaq,Cf,YZgo),(Xaq,Cf,YZgp),(Xaq,Cf,YZgq),(Xaq,Cf, YZgr),(Xaq,Cf,YZgs),(Xaq,Cf,YZgt), (Xar,Ca,YZa),(Xar,Ca,YZb),(Xar,Ca,YZc),(Xar,Ca,YZd),(Xar,Ca,YZe),(Xar,Ca,YZf), (Xar,Ca,YZg),(Xar,Ca,YZh),(Xar,Ca,YZi),(Xar,Ca,YZj),(Xar,Ca,YZk),(Xar,Ca,YZl),(Xar, Ca,YZm),(Xar,Ca,YZn),(Xar,Ca,YZo),(Xar,Ca,YZp),(Xar,Ca,YZq),(Xar,Ca,YZr), (Xar,Ca,YZs),(Xar,Ca,YZt),(Xar,Ca,YZu),(Xar,Ca,YZv),(Xar,Ca,YZw),(Xar,Ca,YZx), (Xar,Ca,YZy),(Xar,Ca,YZz),(Xar,Ca,YZaa),(Xar,Ca,YZab),(Xar,Ca,YZac),(Xar,Ca,YZad), (Xar,Ca,YZae),(Xar,Ca,YZaf),(Xar,Ca,YZag),(Xar,Ca,YZah),(Xar,Ca,YZai),(Xar, Ca,YZaj),(Xar,Ca,YZak),(Xar,Ca,YZal),(Xar,Ca,YZam),(Xar,Ca,YZan),(Xar,Ca,YZao), (Xar,Ca,YZap),(Xar,Ca,YZaq),(Xar,Ca,YZar),(Xar,Ca,YZas),(Xar,Ca,YZat),(Xar, Ca,YZau),(Xar,Ca,YZav),(Xar,Ca,YZaw),(Xar,Ca,YZax),(Xar,Ca,YZay),(Xar,Ca,YZaz), (Xar,Ca,YZba),(Xar,Ca,YZbb),(Xar,Ca,YZbc),(Xar,Ca,YZbd),(Xar,Ca,YZbe),(Xar, Ca,YZbf),(Xar,Ca,YZbg),(Xar,Ca,YZbh),(Xar,Ca,YZbi),(Xar,Ca,YZbj),(Xar,Ca,YZbk), (Xar,Ca,YZbl),(Xar,Ca,YZbm),(Xar,Ca,YZbn),(Xar,Ca,YZbo),(Xar,Ca,YZbp),(Xar, Ca,YZbq),(Xar,Ca,YZbr),(Xar,Ca,YZbs),(Xar,Ca,YZbt),(Xar,Ca,YZbu),(Xar,Ca,YZbv), (Xar,Ca,YZbw),(Xar,Ca,YZbx),(Xar,Ca,YZby),(Xar,Ca,YZbz),(Xar,Ca,YZca),(Xar, Ca,YZcb),(Xar,Ca,YZcc),(Xar,Ca,YZcd),(Xar,Ca,YZce),(Xar,Ca,YZcf),(Xar,Ca,YZcg), (Xar,Ca,YZch),(Xar,Ca,YZci),(Xar,Ca,YZcj),(Xar,Ca,YZck),(Xar,Ca,YZcl),(Xar,Ca, YZcm),(Xar,Ca,YZcn),(Xar,Ca,YZco),(Xar,Ca,YZcp),(Xar,Ca,YZcq),(Xar,Ca,YZcr),(Xar, Ca,YZcs),(Xar,Ca,YZct),(Xar,Ca,YZcu),(Xar,Ca,YZcv),(Xar,Ca,YZcw),(Xar,Ca, YZcx),(Xar,Ca,YZcy),(Xar,Ca,YZcz),(Xar,Ca,YZda),(Xar,Ca,YZdb),(Xar,Ca,YZdc),(Xar, Ca,YZdd),(Xar,Ca,YZde),(Xar,Ca,YZdf),(Xar,Ca,YZdg),(Xar,Ca,YZdh),(Xar,Ca, YZdi),(Xar,Ca,YZdj),(Xar,Ca,YZdk),(Xar,Ca,YZdl),(Xar,Ca,YZdm),(Xar,Ca,YZdn),(Xar, Ca,YZdo),(Xar,Ca,YZdp),(Xar,Ca,YZdq),(Xar,Ca,YZdr),(Xar,Ca,YZds),(Xar,Ca, Yzdt),(Xar,Ca,YZdu),(Xar,Ca,YZdv),(Xar,Ca,YZdw),(Xar,Ca,YZdx),(Xar,Ca,YZdy),(Xar, Ca,YZdz),(Xar,Ca,YZea),(Xar,Ca,YZeb),(Xar,Ca,YZec),(Xar,Ca,YZed),(Xar,Ca, YZee),(Xar,Ca,YZef),(Xar,Ca,YZeg),(Xar,Ca,YZeh),(Xar,Ca,YZei),(Xar,Ca,YZej),(Xar, Ca,YZek),(Xar,Ca,YZel),(Xar,Ca,YZem),(Xar,Ca,YZen),(Xar,Ca,YZeo),(Xar,Ca,YZep), (Xar,Ca,YZeq),(Xar,Ca,YZer),(Xar,Ca,YZes),(Xar,Ca,YZet),(Xar,Ca,YZeu),(Xar, Ca,YZev),(Xar,Ca,YZew),(Xar,Ca,YZex),(Xar,Ca,YZey),(Xar,Ca,YZez),(Xar,Ca,YZfa), (Xar,Ca,YZfb),(Xar,Ca,YZfc),(Xar,Ca,YZfd),(Xar,Ca,YZfe),(Xar,Ca,YZff),(Xar,Ca, YZfg),(Xar,Ca,YZfh),(Xar,Ca,YZfi),(Xar,Ca,YZfj),(Xar,Ca,YZfk),(Xar,Ca,YZfl),(Xar, Ca,YZfm),(Xar,Ca,YZfn),(Xar,Ca,YZfo),(Xar,Ca,YZfp),(Xar,Ca,YZfq),(Xar,Ca,YZfr), (Xar,Ca,YZfs),(Xar,Ca,YZft),(Xar,Ca,YZfu),(Xar,Ca,YZfv),(Xar,Ca,YZfw),(Xar,Ca, YZfx),(Xar,Ca,YZfy),(Xar,Ca,YZfz),(Xar,Ca,YZga),(Xar,Ca,YZgb),(Xar,Ca,YZgc),(Xar, Ca,YZgd),(Xar,Ca,YZge),(Xar,Ca,YZgf),(Xar,Ca,YZgg),(Xar,Ca,YZgh),(Xar,Ca, YZgi),(Xar,Ca,YZgj),(Xar,Ca,YZgk),(Xar,Ca,YZgl),(Xar,Ca,YZgm),(Xar,Ca,YZgn),(Xar, Ca,YZgo),(Xar,Ca,YZgp),(Xar,Ca,YZgq),(Xar,Ca,YZgr),(Xar,Ca,YZgs),(Xar,Ca, YZgt),(Xar,Cb,YZa),(Xar,Cb,YZb),(Xar,Cb,YZc),(Xar,Cb,YZd),(Xar,Cb,YZe),(Xar,Cb, YZf),(Xar,Cb,YZg),(Xar,Cb,YZh),(Xar,Cb,YZi),(Xar,Cb,YZj),(Xar,Cb,YZk),(Xar,Cb, YZl),(Xar,Cb,YZm),(Xar,Cb,YZn),(Xar,Cb,YZo),(Xar,Cb,YZp),(Xar,Cb,YZq),(Xar, Cb,YZr),(Xar,Cb,YZs),(Xar,Cb,YZt),(Xar,Cb,YZu),(Xar,Cb,YZv),(Xar,Cb,YZw),(Xar, Cb,YZx),(Xar,Cb,YZy),(Xar,Cb,YZz),(Xar,Cb,YZaa),(Xar,Cb,YZab),(Xar,Cb,YZac), (Xar,Cb,YZad),(Xar,Cb,YZae),(Xar,Cb,YZaf),(Xar,Cb,YZag),(Xar,Cb,YZah),(Xar,Cb, YZai),(Xar,Cb,YZaj),(Xar,Cb,YZak),(Xar,Cb,YZal),(Xar,Cb,YZam),(Xar,Cb,YZan),(Xar, Cb,YZao),(Xar,Cb,YZap),(Xar,Cb,YZaq),(Xar,Cb,YZar),(Xar,Cb,YZas),(Xar,Cb, YZat),(Xar,Cb,YZau),(Xar,Cb,YZav),(Xar,Cb,YZaw),(Xar,Cb,YZax),(Xar,Cb,YZay),(Xar, Cb,YZaz),(Xar,Cb,YZba),(Xar,Cb,YZbb),(Xar,Cb,YZbc),(Xar,Cb,YZbd),(Xar,Cb, YZbe),(Xar,Cb,YZbf),(Xar,Cb,YZbg),(Xar,Cb,YZbh),(Xar,Cb,YZbi),(Xar,Cb,YZbj),(Xar, Cb,YZbk),(Xar,Cb,YZbl),(Xar,Cb,YZbm),(Xar,Cb,YZbn),(Xar,Cb,YZbo),(Xar,Cb, YZbp),(Xar,Cb,YZbq),(Xar,Cb,YZbr),(Xar,Cb,YZbs),(Xar,Cb,YZbt),(Xar,Cb,YZbu), (Xar,Cb,YZbv),(Xar,Cb,YZbw),(Xar,Cb,YZbx),(Xar,Cb,YZby),(Xar,Cb,YZbz),(Xar, Cb,YZca),(Xar,Cb,YZcb),(Xar,Cb,YZcc),(Xar,Cb,YZcd),(Xar,Cb,YZce),(Xar,Cb,YZcf), (Xar,Cb,YZcg),(Xar,Cb,YZch),(Xar,Cb,YZci),(Xar,Cb,YZcj),(Xar,Cb,YZck),(Xar, Cb,YZcl),(Xar,Cb,YZcm),(Xar,Cb,YZcn),(Xar,Cb,YZco),(Xar,Cb,YZcp),(Xar,Cb,YZcq), (Xar,Cb,YZcr),(Xar,Cb,YZcs),(Xar,Cb,YZct),(Xar,Cb,YZcu),(Xar,Cb,YZcv),(Xar, Cb,YZcw),(Xar,Cb,YZcx),(Xar,Cb,YZcy),(Xar,Cb,YZcz),(Xar,Cb,YZda),(Xar,Cb,YZdb), (Xar,Cb,YZdc),(Xar,Cb,YZdd),(Xar,Cb,YZde),(Xar,Cb,YZdf),(Xar,Cb,YZdg),(Xar, Cb,YZdh),(Xar,Cb,YZdi),(Xar,Cb,YZdj),(Xar,Cb,YZdk),(Xar,Cb,YZdl),(Xar,Cb,YZdm), (Xar,Cb,YZdn),(Xar,Cb,YZdo),(Xar,Cb,YZdp),(Xar,Cb,YZdq),(Xar,Cb,YZdr),(Xar, Cb,YZds),(Xar,Cb,Yzdt),(Xar,Cb,YZdu),(Xar,Cb,YZdv),(Xar,Cb,YZdw),(Xar,Cb,YZdx), (Xar,Cb,YZdy),(Xar,Cb,YZdz),(Xar,Cb,YZea),(Xar,Cb,YZeb),(Xar,Cb,YZec),(Xar, Cb,YZed),(Xar,Cb,YZee),(Xar,Cb,YZef),(Xar,Cb,YZeg),(Xar,Cb,YZeh),(Xar,Cb,YZei), (Xar,Cb,YZej),(Xar,Cb,YZek),(Xar,Cb,YZel),(Xar,Cb,YZem),(Xar,Cb,YZen),(Xar, Cb,YZeo),(Xar,Cb,YZep),(Xar,Cb,YZeq),(Xar,Cb,YZer),(Xar,Cb,YZes),(Xar,Cb,YZet), (Xar,Cb,YZeu),(Xar,Cb,YZev),(Xar,Cb,YZew),(Xar,Cb,YZex),(Xar,Cb,YZey),(Xar, Cb,YZez),(Xar,Cb,YZfa),(Xar,Cb,YZfb),(Xar,Cb,YZfc),(Xar,Cb,YZfd),(Xar,Cb,YZfe), (Xar,Cb,YZff),(Xar,Cb,YZfg),(Xar,Cb,YZfh),(Xar,Cb,YZfi),(Xar,Cb,YZfj),(Xar,Cb, YZfk),(Xar,Cb,YZfl),(Xar,Cb,YZfm),(Xar,Cb,YZfn),(Xar,Cb,YZfo),(Xar,Cb,YZfp), (Xar,Cb,YZfq),(Xar,Cb,YZfr),(Xar,Cb,YZfs),(Xar,Cb,YZft),(Xar,Cb,YZfu),(Xar,Cb,YZfv), (Xar,Cb,YZfw),(Xar,Cb,YZfx),(Xar,Cb,YZfy),(Xar,Cb,YZfz),(Xar,Cb,YZga),(Xar, Cb,YZgb),(Xar,Cb,YZgc),(Xar,Cb,YZgd),(Xar,Cb,YZge),(Xar,Cb,YZgf),(Xar,Cb,YZgg), (Xar,Cb,YZgh),(Xar,Cb,YZgi),(Xar,Cb,YZgj),(Xar,Cb,YZgk),(Xar,Cb,YZgl),(Xar, Cb,YZgm),(Xar,Cb,YZgn),(Xar,Cb,YZgo),(Xar,Cb,YZgp),(Xar,Cb,YZgq),(Xar,Cb, YZgr),(Xar,Cb,YZgs),(Xar,Cb,YZgt),(Xar,Cc,YZa),(Xar,Cc,YZb),(Xar,Cc,YZc),(Xar, Cc,YZd),(Xar,Cc,YZe),(Xar,Cc,YZf),(Xar,Cc,YZg),(Xar,Cc,YZh),(Xar,Cc,YZi),(Xar, Cc,YZj),(Xar,Cc,YZk),(Xar,Cc,YZl),(Xar,Cc,YZm),(Xar,Cc,YZn),(Xar,Cc,YZo),(Xar, Cc,YZp),(Xar,Cc,YZq),(Xar,Cc,YZr),(Xar,Cc,YZs),(Xar,Cc,YZt),(Xar,Cc,YZu),(Xar, Cc,YZv),(Xar,Cc,YZw),(Xar,Cc,YZx),(Xar,Cc,YZy),(Xar,Cc,YZz),(Xar,Cc,YZaa),(Xar, Cc,YZab),(Xar,Cc,YZac),(Xar,Cc,YZad),(Xar,Cc,YZae),(Xar,Cc,YZaf),(Xar,Cc,YZag), (Xar,Cc,YZah),(Xar,Cc,YZai),(Xar,Cc,YZaj),(Xar,Cc,YZak),(Xar,Cc,YZal),(Xar, Cc,YZam),(Xar,Cc,YZan),(Xar,Cc,YZao),(Xar,Cc,YZap),(Xar,Cc,YZaq),(Xar,Cc,YZar), (Xar,Cc,YZas),(Xar,Cc,YZat),(Xar,Cc,YZau),(Xar,Cc,YZav),(Xar,Cc,YZaw),(Xar, Cc,YZax),(Xar,Cc,YZay),(Xar,Cc,YZaz),(Xar,Cc,YZba),(Xar,Cc,YZbb),(Xar,Cc,YZbc), (Xar,Cc,YZbd),(Xar,Cc,YZbe),(Xar,Cc,YZbf),(Xar,Cc,YZbg),(Xar,Cc,YZbh),(Xar, Cc,YZbi),(Xar,Cc,YZbj),(Xar,Cc,YZbk),(Xar,Cc,YZbl),(Xar,Cc,YZbm),(Xar,Cc,YZbn), (Xar,Cc,YZbo),(Xar,Cc,YZbp),(Xar,Cc,YZbq),(Xar,Cc,YZbr),(Xar,Cc,YZbs),(Xar, Cc,YZbt),(Xar,Cc,YZbu),(Xar,Cc,YZbv),(Xar,Cc,YZbw),(Xar,Cc,YZbx),(Xar,Cc,YZby), (Xar,Cc,YZbz),(Xar,Cc,YZca),(Xar,Cc,YZcb),(Xar,Cc,YZcc),(Xar,Cc,YZcd),(Xar, Cc,YZce),(Xar,Cc,YZcf),(Xar,Cc,YZcg),(Xar,Cc,YZch),(Xar,Cc,YZci),(Xar,Cc,YZcj), (Xar,Cc,YZck),(Xar,Cc,YZcl),(Xar,Cc,YZcm),(Xar,Cc,YZcn),(Xar,Cc,YZco),(Xar,Cc, YZcp),(Xar,Cc,YZcq),(Xar,Cc,YZcr),(Xar,Cc,YZcs),(Xar,Cc,YZct),(Xar,Cc,YZcu),(Xar, Cc,YZcv),(Xar,Cc,YZcw),(Xar,Cc,YZcx),(Xar,Cc,YZcy),(Xar,Cc,YZcz),(Xar,Cc, YZda),(Xar,Cc,YZdb),(Xar,Cc,YZdc),(Xar,Cc,YZdd),(Xar,Cc,YZde),(Xar,Cc,YZdf),(Xar, Cc,YZdg),(Xar,Cc,YZdh),(Xar,Cc,YZdi),(Xar,Cc,YZdj),(Xar,Cc,YZdk),(Xar,Cc, YZdl),(Xar,Cc,YZdm),(Xar,Cc,YZdn),(Xar,Cc,YZdo),(Xar,Cc,YZdp),(Xar,Cc,YZdq), (Xar,Cc,YZdr),(Xar,Cc,YZds),(Xar,Cc,Yzdt),(Xar,Cc,YZdu),(Xar,Cc,YZdv),(Xar,Cc, YZdw),(Xar,Cc,YZdx),(Xar,Cc,YZdy),(Xar,Cc,YZdz),(Xar,Cc,YZea),(Xar,Cc,YZeb), (Xar,Cc,YZec),(Xar,Cc,YZed),(Xar,Cc,YZee),(Xar,Cc,YZef),(Xar,Cc,YZeg),(Xar,Cc, YZeh),(Xar,Cc,YZei),(Xar,Cc,YZej),(Xar,Cc,YZek),(Xar,Cc,YZel),(Xar,Cc,YZem),(Xar, Cc,YZen),(Xar,Cc,YZeo),(Xar,Cc,YZep),(Xar,Cc,YZeq),(Xar,Cc,YZer),(Xar,Cc, YZes),(Xar,Cc,YZet),(Xar,Cc,YZeu),(Xar,Cc,YZev),(Xar,Cc,YZew),(Xar,Cc,YZex),(Xar, Cc,YZey),(Xar,Cc,YZez),(Xar,Cc,YZfa),(Xar,Cc,YZfb),(Xar,Cc,YZfc),(Xar,Cc,YZfd), (Xar,Cc,YZfe),(Xar,Cc,YZff),(Xar,Cc,YZfg),(Xar,Cc,YZfh),(Xar,Cc,YZfi),(Xar, Cc,YZfj),(Xar,Cc,YZfk),(Xar,Cc,YZfl),(Xar,Cc,YZfm),(Xar,Cc,YZfn),(Xar,Cc,YZfo), (Xar,Cc,YZfp),(Xar,Cc,YZfq),(Xar,Cc,YZfr),(Xar,Cc,YZfs),(Xar,Cc,YZft),(Xar,Cc,YZfu), (Xar,Cc,YZfv),(Xar,Cc,YZfw),(Xar,Cc,YZfx),(Xar,Cc,YZfy),(Xar,Cc,YZfz),(Xar, Cc,YZga),(Xar,Cc,YZgb),(Xar,Cc,YZgc),(Xar,Cc,YZgd),(Xar,Cc,YZge),(Xar,Cc,YZgf), (Xar,Cc,YZgg),(Xar,Cc,YZgh),(Xar,Cc,YZgi),(Xar,Cc,YZgj),(Xar,Cc,YZgk),(Xar, Cc,YZgl),(Xar,Cc,YZgm),(Xar,Cc,YZgn),(Xar,Cc,YZgo),(Xar,Cc,YZgp),(Xar,Cc,YZgq), (Xar,Cc,YZgr),(Xar,Cc,YZgs),(Xar,Cc,YZgt),(Xar,Cd,YZa),(Xar,Cd,YZb),(Xar,Cd, YZc),(Xar,Cd,YZd),(Xar,Cd,YZe),(Xar,Cd,YZf),(Xar,Cd,YZg),(Xar,Cd,YZh),(Xar, Cd,YZi),(Xar,Cd,YZj),(Xar,Cd,YZk),(Xar,Cd,YZl),(Xar,Cd,YZm),(Xar,Cd,YZn),(Xar, Cd,YZo),(Xar,Cd,YZp),(Xar,Cd,YZq),(Xar,Cd,YZr),(Xar,Cd,YZs),(Xar,Cd,YZt),(Xar, Cd,YZu),(Xar,Cd,YZv),(Xar,Cd,YZw),(Xar,Cd,YZx),(Xar,Cd,YZy),(Xar,Cd,YZz),(Xar, Cd,YZaa),(Xar,Cd,YZab),(Xar,Cd,YZac),(Xar,Cd,YZad),(Xar,Cd,YZae),(Xar,Cd,YZaf), (Xar,Cd,YZag),(Xar,Cd,YZah),(Xar,Cd,YZai),(Xar,Cd,YZaj),(Xar,Cd,YZak),(Xar, Cd,YZal),(Xar,Cd,YZam),(Xar,Cd,YZan),(Xar,Cd,YZao),(Xar,Cd,YZap),(Xar,Cd,YZaq), (Xar,Cd,YZar),(Xar,Cd,YZas),(Xar,Cd,YZat),(Xar,Cd,YZau),(Xar,Cd,YZav),(Xar, Cd,YZaw),(Xar,Cd,YZax),(Xar,Cd,YZay),(Xar,Cd,YZaz),(Xar,Cd,YZba),(Xar,Cd,YZbb), (Xar,Cd,YZbc),(Xar,Cd,YZbd),(Xar,Cd,YZbe),(Xar,Cd,YZbf),(Xar,Cd,YZbg),(Xar, Cd,YZbh),(Xar,Cd,YZbi),(Xar,Cd,YZbj),(Xar,Cd,YZbk),(Xar,Cd,YZbl),(Xar,Cd, YZbm),(Xar,Cd,YZbn),(Xar,Cd,YZbo),(Xar,Cd,YZbp),(Xar,Cd,YZbq),(Xar,Cd,YZbr), (Xar,Cd,YZbs),(Xar,Cd,YZbt),(Xar,Cd,YZbu),(Xar,Cd,YZbv),(Xar,Cd,YZbw),(Xar,Cd, YZbx),(Xar,Cd,YZby),(Xar,Cd,YZbz),(Xar,Cd,YZca),(Xar,Cd,YZcb),(Xar,Cd,YZcc), (Xar,Cd,YZcd),(Xar,Cd,YZce),(Xar,Cd,YZcf),(Xar,Cd,YZcg),(Xar,Cd,YZch),(Xar,Cd, YZci),(Xar,Cd,YZcj),(Xar,Cd,YZck),(Xar,Cd,YZcl),(Xar,Cd,YZcm),(Xar,Cd,YZcn), (Xar,Cd,YZco),(Xar,Cd,YZcp),(Xar,Cd,YZcq),(Xar,Cd,YZcr),(Xar,Cd,YZcs),(Xar,Cd, YZct),(Xar,Cd,YZcu),(Xar,Cd,YZcv),(Xar,Cd,YZcw),(Xar,Cd,YZcx),(Xar,Cd,YZcy), (Xar,Cd,YZcz),(Xar,Cd,YZda),(Xar,Cd,YZdb),(Xar,Cd,YZdc),(Xar,Cd,YZdd),(Xar, Cd,YZde),(Xar,Cd,YZdf),(Xar,Cd,YZdg),(Xar,Cd,YZdh),(Xar,Cd,YZdi),(Xar,Cd,YZdj), (Xar,Cd,YZdk),(Xar,Cd,YZdl),(Xar,Cd,YZdm),(Xar,Cd,YZdn),(Xar,Cd,YZdo),(Xar, Cd,YZdp),(Xar,Cd,YZdq),(Xar,Cd,YZdr),(Xar,Cd,YZds),(Xar,Cd,Yzdt),(Xar,Cd,YZdu), (Xar,Cd,YZdv),(Xar,Cd,YZdw),(Xar,Cd,YZdx),(Xar,Cd,YZdy),(Xar,Cd,YZdz),(Xar, Cd,YZea),(Xar,Cd,YZeb),(Xar,Cd,YZec),(Xar,Cd,YZed),(Xar,Cd,YZee),(Xar,Cd,YZef), (Xar,Cd,YZeg),(Xar,Cd,YZeh),(Xar,Cd,YZei),(Xar,Cd,YZej),(Xar,Cd,YZek),(Xar, Cd,YZel),(Xar,Cd,YZem),(Xar,Cd,YZen),(Xar,Cd,YZeo),(Xar,Cd,YZep),(Xar,Cd,YZeq), (Xar,Cd,YZer),(Xar,Cd,YZes),(Xar,Cd,YZet),(Xar,Cd,YZeu),(Xar,Cd,YZev),(Xar, Cd,YZew),(Xar,Cd,YZex),(Xar,Cd,YZey),(Xar,Cd,YZez),(Xar,Cd,YZfa),(Xar,Cd,YZfb), (Xar,Cd,YZfc),(Xar,Cd,YZfd),(Xar,Cd,YZfe),(Xar,Cd,YZff),(Xar,Cd,YZfg),(Xar,Cd, YZfh),(Xar,Cd,YZfi),(Xar,Cd,YZfj),(Xar,Cd,YZfk),(Xar,Cd,YZfl),(Xar,Cd,YZfm),(Xar, Cd,YZfn),(Xar,Cd,YZfo),(Xar,Cd,YZfp),(Xar,Cd,YZfq),(Xar,Cd,YZfr),(Xar,Cd,YZfs), (Xar,Cd,YZft),(Xar,Cd,YZfu),(Xar,Cd,YZfv),(Xar,Cd,YZfw),(Xar,Cd,YZfx),(Xar, Cd,YZfy),(Xar,Cd,YZfz),(Xar,Cd,YZga),(Xar,Cd,YZgb),(Xar,Cd,YZgc),(Xar,Cd,YZgd), (Xar,Cd,YZge),(Xar,Cd,YZgf),(Xar,Cd,YZgg),(Xar,Cd,YZgh),(Xar,Cd,YZgi),(Xar, Cd,YZgj),(Xar,Cd,YZgk),(Xar,Cd,YZgl),(Xar,Cd,YZgm),(Xar,Cd,YZgn),(Xar,Cd,YZgo), (Xar,Cd,YZgp),(Xar,Cd,YZgq),(Xar,Cd,YZgr),(Xar,Cd,YZgs),(Xar,Cd,YZgt),(Xar, Ce,YZa),(Xar,Ce,YZb),(Xar,Ce,YZc),(Xar,Ce,YZd),(Xar,Ce,YZe),(Xar,Ce,YZf),(Xar, Ce,YZg),(Xar,Ce,YZh),(Xar,Ce,YZi),(Xar,Ce,YZj),(Xar,Ce,YZk),(Xar,Ce,YZl),(Xar, Ce,YZm),(Xar,Ce,YZn),(Xar,Ce,YZo),(Xar,Ce,YZp),(Xar,Ce,YZq),(Xar,Ce,YZr),(Xar, Ce,YZs),(Xar,Ce,YZt),(Xar,Ce,YZu),(Xar,Ce,YZv),(Xar,Ce,YZw),(Xar,Ce,YZx),(Xar, Ce,YZy),(Xar,Ce,YZz),(Xar,Ce,YZaa),(Xar,Ce,YZab),(Xar,Ce,YZac),(Xar,Ce,YZad), (Xar,Ce,YZae),(Xar,Ce,YZaf),(Xar,Ce,YZag),(Xar,Ce,YZah),(Xar,Ce,YZai),(Xar, Ce,YZaj),(Xar,Ce,YZak),(Xar,Ce,YZal),(Xar,Ce,YZam),(Xar,Ce,YZan),(Xar,Ce,YZao), (Xar,Ce,YZap),(Xar,Ce,YZaq),(Xar,Ce,YZar),(Xar,Ce,YZas),(Xar,Ce,YZat),(Xar,Ce, YZau),(Xar,Ce,YZav),(Xar,Ce,YZaw),(Xar,Ce,YZax),(Xar,Ce,YZay),(Xar,Ce,YZaz), (Xar,Ce,YZba),(Xar,Ce,YZbb),(Xar,Ce,YZbc),(Xar,Ce,YZbd),(Xar,Ce,YZbe),(Xar,Ce, YZbf),(Xar,Ce,YZbg),(Xar,Ce,YZbh),(Xar,Ce,YZbi),(Xar,Ce,YZbj),(Xar,Ce,YZbk),(Xar, Ce,YZbl),(Xar,Ce,YZbm),(Xar,Ce,YZbn),(Xar,Ce,YZbo),(Xar,Ce,YZbp),(Xar,Ce, YZbq),(Xar,Ce,YZbr),(Xar,Ce,YZbs),(Xar,Ce,YZbt),(Xar,Ce,YZbu),(Xar,Ce,YZbv),(Xar, Ce,YZbw),(Xar,Ce,YZbx),(Xar,Ce,YZby),(Xar,Ce,YZbz),(Xar,Ce,YZca),(Xar,Ce, YZcb),(Xar,Ce,YZcc),(Xar,Ce,YZcd),(Xar,Ce,YZce),(Xar,Ce,YZcf),(Xar,Ce,YZcg),(Xar, Ce,YZch),(Xar,Ce,YZci),(Xar,Ce,YZcj),(Xar,Ce,YZck),(Xar,Ce,YZcl),(Xar,Ce,YZcm), (Xar,Ce,YZcn),(Xar,Ce,YZco),(Xar,Ce,YZcp),(Xar,Ce,YZcq),(Xar,Ce,YZcr),(Xar, Ce,YZcs),(Xar,Ce,YZct),(Xar,Ce,YZcu),(Xar,Ce,YZcv),(Xar,Ce,YZcw),(Xar,Ce,YZcx), (Xar,Ce,YZcy),(Xar,Ce,YZcz),(Xar,Ce,YZda),(Xar,Ce,YZdb),(Xar,Ce,YZdc),(Xar, Ce,YZdd),(Xar,Ce,YZde),(Xar,Ce,YZdf),(Xar,Ce,YZdg),(Xar,Ce,YZdh),(Xar,Ce,YZdi), (Xar,Ce,YZdj),(Xar,Ce,YZdk),(Xar,Ce,YZdl),(Xar,Ce,YZdm),(Xar,Ce,YZdn),(Xar, Ce,YZdo),(Xar,Ce,YZdp),(Xar,Ce,YZdq),(Xar,Ce,YZdr),(Xar,Ce,YZds),(Xar,Ce,Yzdt), (Xar,Ce,YZdu),(Xar,Ce,YZdv),(Xar,Ce,YZdw),(Xar,Ce,YZdx),(Xar,Ce,YZdy),(Xar, Ce,YZdz),(Xar,Ce,YZea),(Xar,Ce,YZeb),(Xar,Ce,YZec),(Xar,Ce,YZed),(Xar,Ce,YZee), (Xar,Ce,YZef),(Xar,Ce,YZeg),(Xar,Ce,YZeh),(Xar,Ce,YZei),(Xar,Ce,YZej),(Xar, Ce,YZek),(Xar,Ce,YZel),(Xar,Ce,YZem),(Xar,Ce,YZen),(Xar,Ce,YZeo),(Xar,Ce,YZep), (Xar,Ce,YZeq),(Xar,Ce,YZer),(Xar,Ce,YZes),(Xar,Ce,YZet),(Xar,Ce,YZeu),(Xar,Ce, YZev),(Xar,Ce,YZew),(Xar,Ce,YZex),(Xar,Ce,YZey),(Xar,Ce,YZez),(Xar,Ce,YZfa), (Xar,Ce,YZfb),(Xar,Ce,YZfc),(Xar,Ce,YZfd),(Xar,Ce,YZfe),(Xar,Ce,YZff),(Xar,Ce, YZfg),(Xar,Ce,YZfh),(Xar,Ce,YZfi),(Xar,Ce,YZfj),(Xar,Ce,YZfk),(Xar,Ce,YZfl),(Xar, Ce,YZfm),(Xar,Ce,YZfn),(Xar,Ce,YZfo),(Xar,Ce,YZfp),(Xar,Ce,YZfq),(Xar,Ce,YZfr), (Xar,Ce,YZfs),(Xar,Ce,YZft),(Xar,Ce,YZfu),(Xar,Ce,YZfv),(Xar,Ce,YZfw),(Xar,Ce, YZfx),(Xar,Ce,YZfy),(Xar,Ce,YZfz),(Xar,Ce,YZga),(Xar,Ce,YZgb),(Xar,Ce,YZgc),(Xar, Ce,YZgd),(Xar,Ce,YZge),(Xar,Ce,YZgf),(Xar,Ce,YZgg),(Xar,Ce,YZgh),(Xar,Ce, YZgi),(Xar,Ce,YZgj),(Xar,Ce,YZgk),(Xar,Ce,YZgl),(Xar,Ce,YZgm),(Xar,Ce,YZgn),(Xar, Ce,YZgo),(Xar,Ce,YZgp),(Xar,Ce,YZgq),(Xar,Ce,YZgr),(Xar,Ce,YZgs),(Xar,Ce, YZgt),(Xar,Cf,YZa),(Xar,Cf,YZb),(Xar,Cf,YZc),(Xar,Cf,YZd),(Xar,Cf,YZe),(Xar,Cf, YZf),(Xar,Cf,YZg),(Xar,Cf,YZh),(Xar,Cf,YZi),(Xar,Cf,YZj),(Xar,Cf,YZk),(Xar,Cf,YZl), (Xar,Cf,YZm),(Xar,Cf,YZn),(Xar,Cf,YZo),(Xar,Cf,YZp),(Xar,Cf,YZq),(Xar,Cf,YZr), (Xar,Cf,YZs),(Xar,Cf,YZt),(Xar,Cf,YZu),(Xar,Cf,YZv),(Xar,Cf,YZw),(Xar,Cf,YZx), (Xar,Cf,YZy),(Xar,Cf,YZz),(Xar,Cf,YZaa),(Xar,Cf,YZab),(Xar,Cf,YZac),(Xar,Cf, YZad),(Xar,Cf,YZae),(Xar,Cf,YZaf),(Xar,Cf,YZag),(Xar,Cf,YZah),(Xar,Cf,YZai),(Xar, Cf,YZaj),(Xar,Cf,YZak),(Xar,Cf,YZal),(Xar,Cf,YZam),(Xar,Cf,YZan),(Xar,Cf,YZao), (Xar,Cf,YZap),(Xar,Cf,YZaq),(Xar,Cf,YZar),(Xar,Cf,YZas),(Xar,Cf,YZat),(Xar,Cf, YZau),(Xar,Cf,YZav),(Xar,Cf,YZaw),(Xar,Cf,YZax),(Xar,Cf,YZay),(Xar,Cf,YZaz),(Xar, Cf,YZba),(Xar,Cf,YZbb),(Xar,Cf,YZbc),(Xar,Cf,YZbd),(Xar,Cf,YZbe),(Xar,Cf,YZbf), (Xar,Cf,YZbg),(Xar,Cf,YZbh),(Xar,Cf,YZbi),(Xar,Cf,YZbj),(Xar,Cf,YZbk),(Xar, Cf,YZbl),(Xar,Cf,YZbm),(Xar,Cf,YZbn),(Xar,Cf,YZbo),(Xar,Cf,YZbp),(Xar,Cf,YZbq), (Xar,Cf,YZbr),(Xar,Cf,YZbs),(Xar,Cf,YZbt),(Xar,Cf,YZbu),(Xar,Cf,YZbv),(Xar,Cf, YZbw),(Xar,Cf,YZbx),(Xar,Cf,YZby),(Xar,Cf,YZbz),(Xar,Cf,YZca),(Xar,Cf,YZcb),(Xar, Cf,YZcc),(Xar,Cf,YZcd),(Xar,Cf,YZce),(Xar,Cf,YZcf),(Xar,Cf,YZcg),(Xar,Cf,YZch), (Xar,Cf,YZci),(Xar,Cf,YZcj),(Xar,Cf,YZck),(Xar,Cf,YZcl),(Xar,Cf,YZcm),(Xar, Cf,YZcn),(Xar,Cf,YZco),(Xar,Cf,YZcp),(Xar,Cf,YZcq),(Xar,Cf,YZcr),(Xar,Cf,YZcs), (Xar,Cf,YZct),(Xar,Cf,YZcu),(Xar,Cf,YZcv),(Xar,Cf,YZcw),(Xar,Cf,YZcx),(Xar,Cf, YZcy),(Xar,Cf,YZcz),(Xar,Cf,YZda),(Xar,Cf,YZdb),(Xar,Cf,YZdc),(Xar,Cf,YZdd),(Xar, Cf,YZde),(Xar,Cf,YZdf),(Xar,Cf,YZdg),(Xar,Cf,YZdh),(Xar,Cf,YZdi),(Xar,Cf,YZdj), (Xar,Cf,YZdk),(Xar,Cf,YZdl),(Xar,Cf,YZdm),(Xar,Cf,YZdn),(Xar,Cf,YZdo),(Xar,Cf, YZdp),(Xar,Cf,YZdq),(Xar,Cf,YZdr),(Xar,Cf,YZds),(Xar,Cf,Yzdt),(Xar,Cf,YZdu),(Xar, Cf,YZdv),(Xar,Cf,YZdw),(Xar,Cf,YZdx),(Xar,Cf,YZdy),(Xar,Cf,YZdz),(Xar,Cf, YZea),(Xar,Cf,YZeb),(Xar,Cf,YZec),(Xar,Cf,YZed),(Xar,Cf,YZee),(Xar,Cf,YZef),(Xar, Cf,YZeg),(Xar,Cf,YZeh),(Xar,Cf,YZei),(Xar,Cf,YZej),(Xar,Cf,YZek),(Xar,Cf,YZel), (Xar,Cf,YZem),(Xar,Cf,YZen),(Xar,Cf,YZeo),(Xar,Cf,YZep),(Xar,Cf,YZeq),(Xar,Cf, YZer),(Xar,Cf,YZes),(Xar,Cf,YZet),(Xar,Cf,YZeu),(Xar,Cf,YZev),(Xar,Cf,YZew),(Xar, Cf,YZex),(Xar,Cf,YZey),(Xar,Cf,YZez),(Xar,Cf,YZfa),(Xar,Cf,YZfb),(Xar,Cf,YZfc), (Xar,Cf,YZfd),(Xar,Cf,YZfe),(Xar,Cf,YZff),(Xar,Cf,YZfg),(Xar,Cf,YZfh),(Xar,Cf, YZfi),(Xar,Cf,YZfj),(Xar,Cf,YZfk),(Xar,Cf,YZfl),(Xar,Cf,YZfm),(Xar,Cf,YZfn),(Xar, Cf,YZfo),(Xar,Cf,YZfp),(Xar,Cf,YZfq),(Xar,Cf,YZfr),(Xar,Cf,YZfs),(Xar,Cf,YZft),(Xar, Cf,YZfu),(Xar,Cf,YZfv),(Xar,Cf,YZfw),(Xar,Cf,YZfx),(Xar,Cf,YZfy),(Xar,Cf,YZfz), (Xar,Cf,YZga),(Xar,Cf,YZgb),(Xar,Cf,YZgc),(Xar,Cf,YZgd),(Xar,Cf,YZge),(Xar,Cf, YZgf),(Xar,Cf,YZgg),(Xar,Cf,YZgh),(Xar,Cf,YZgi),(Xar,Cf,YZgj),(Xar,Cf,YZgk),(Xar, Cf,YZgl),(Xar,Cf,YZgm),(Xar,Cf,YZgn),(Xar,Cf,YZgo),(Xar,Cf,YZgp),(Xar,Cf, YZgq),(Xar,Cf,YZgr),(Xar,Cf,YZgs),(Xar,Cf,YZgt), (Xas,Ca,YZa),(Xas,Ca,YZb),(Xas,Ca,YZc),(Xas,Ca,YZd),(Xas,Ca,YZe),(Xas,Ca,YZf), (Xas,Ca,YZg),(Xas,Ca,YZh),(Xas,Ca,YZi),(Xas,Ca,YZj),(Xas,Ca,YZk),(Xas,Ca,YZl), (Xas,Ca,YZm),(Xas,Ca,YZn),(Xas,Ca,YZo),(Xas,Ca,YZp),(Xas,Ca,YZq),(Xas,Ca,YZr), (Xas,Ca,YZs),(Xas,Ca,YZt),(Xas,Ca,YZu),(Xas,Ca,YZv),(Xas,Ca,YZw),(Xas,Ca,YZx), (Xas,Ca,YZy),(Xas,Ca,YZz),(Xas,Ca,YZaa),(Xas,Ca,YZab),(Xas,Ca,YZac),(Xas, Ca,YZad),(Xas,Ca,YZae),(Xas,Ca,YZaf),(Xas,Ca,YZag),(Xas,Ca,YZah),(Xas,Ca,YZai), (Xas,Ca,YZaj),(Xas,Ca,YZak),(Xas,Ca,YZal),(Xas,Ca,YZam),(Xas,Ca,YZan),(Xas, Ca,YZao),(Xas,Ca,YZap),(Xas,Ca,YZaq),(Xas,Ca,YZar),(Xas,Ca,YZas),(Xas,Ca,YZat), (Xas,Ca,YZau),(Xas,Ca,YZav),(Xas,Ca,YZaw),(Xas,Ca,YZax),(Xas,Ca,YZay),(Xas, Ca,YZaz),(Xas,Ca,YZba),(Xas,Ca,YZbb),(Xas,Ca,YZbc),(Xas,Ca,YZbd),(Xas,Ca,YZbe), (Xas,Ca,YZbf),(Xas,Ca,YZbg),(Xas,Ca,YZbh),(Xas,Ca,YZbi),(Xas,Ca,YZbj),(Xas, Ca,YZbk),(Xas,Ca,YZbl),(Xas,Ca,YZbm),(Xas,Ca,YZbn),(Xas,Ca,YZbo),(Xas,Ca,YZbp), (Xas,Ca,YZbq),(Xas,Ca,YZbr),(Xas,Ca,YZbs),(Xas,Ca,YZbt),(Xas,Ca,YZbu),(Xas, Ca,YZbv),(Xas,Ca,YZbw),(Xas,Ca,YZbx),(Xas,Ca,YZby),(Xas,Ca,YZbz),(Xas,Ca, YZca),(Xas,Ca,YZcb),(Xas,Ca,YZcc),(Xas,Ca,YZcd),(Xas,Ca,YZce),(Xas,Ca,YZcf),(Xas, Ca,YZcg),(Xas,Ca,YZch),(Xas,Ca,YZci),(Xas,Ca,YZcj),(Xas,Ca,YZck),(Xas,Ca, YZcl),(Xas,Ca,YZcm),(Xas,Ca,YZcn),(Xas,Ca,YZco),(Xas,Ca,YZcp),(Xas,Ca,YZcq), (Xas,Ca,YZcr),(Xas,Ca,YZcs),(Xas,Ca,YZct),(Xas,Ca,YZcu),(Xas,Ca,YZcv),(Xas,Ca, YZcw),(Xas,Ca,YZcx),(Xas,Ca,YZcy),(Xas,Ca,YZcz),(Xas,Ca,YZda),(Xas,Ca,YZdb), (Xas,Ca,YZdc),(Xas,Ca,YZdd),(Xas,Ca,YZde),(Xas,Ca,YZdf),(Xas,Ca,YZdg),(Xas,Ca, YZdh),(Xas,Ca,YZdi),(Xas,Ca,YZdj),(Xas,Ca,YZdk),(Xas,Ca,YZdl),(Xas,Ca,YZdm), (Xas,Ca,YZdn),(Xas,Ca,YZdo),(Xas,Ca,YZdp),(Xas,Ca,YZdq),(Xas,Ca,YZdr),(Xas, Ca,YZds),(Xas,Ca,Yzdt),(Xas,Ca,YZdu),(Xas,Ca,YZdv),(Xas,Ca,YZdw),(Xas,Ca,YZdx), (Xas,Ca,YZdy),(Xas,Ca,YZdz),(Xas,Ca,YZea),(Xas,Ca,YZeb),(Xas,Ca,YZec),(Xas, Ca,YZed),(Xas,Ca,YZee),(Xas,Ca,YZef),(Xas,Ca,YZeg),(Xas,Ca,YZeh),(Xas,Ca,YZei), (Xas,Ca,YZej),(Xas,Ca,YZek),(Xas,Ca,YZel),(Xas,Ca,YZem),(Xas,Ca,YZen),(Xas, Ca,YZeo),(Xas,Ca,YZep),(Xas,Ca,YZeq),(Xas,Ca,YZer),(Xas,Ca,YZes),(Xas,Ca,YZet), (Xas,Ca,YZeu),(Xas,Ca,YZev),(Xas,Ca,YZew),(Xas,Ca,YZex),(Xas,Ca,YZey),(Xas, Ca,YZez),(Xas,Ca,YZfa),(Xas,Ca,YZfb),(Xas,Ca,YZfc),(Xas,Ca,YZfd),(Xas,Ca,YZfe), (Xas,Ca,YZff),(Xas,Ca,YZfg),(Xas,Ca,YZfh),(Xas,Ca,YZfi),(Xas,Ca,YZfj),(Xas,Ca, YZfk),(Xas,Ca,YZfl),(Xas,Ca,YZfm),(Xas,Ca,YZfn),(Xas,Ca,YZfo),(Xas,Ca,YZfp),(Xas, Ca,YZfq),(Xas,Ca,YZfr),(Xas,Ca,YZfs),(Xas,Ca,YZft),(Xas,Ca,YZfu),(Xas,Ca,YZfv), (Xas,Ca,YZfw),(Xas,Ca,YZfx),(Xas,Ca,YZfy),(Xas,Ca,YZfz),(Xas,Ca,YZga),(Xas, Ca,YZgb),(Xas,Ca,YZgc),(Xas,Ca,YZgd),(Xas,Ca,YZge),(Xas,Ca,YZgf),(Xas,Ca,YZgg), (Xas,Ca,YZgh),(Xas,Ca,YZgi),(Xas,Ca,YZgj),(Xas,Ca,YZgk),(Xas,Ca,YZgl),(Xas, Ca,YZgm),(Xas,Ca,YZgn),(Xas,Ca,YZgo),(Xas,Ca,YZgp),(Xas,Ca,YZgq),(Xas,Ca, YZgr),(Xas,Ca,YZgs),(Xas,Ca,YZgt),(Xas,Cb,YZa),(Xas,Cb,YZb),(Xas,Cb,YZc),(Xas, Cb,YZd),(Xas,Cb,YZe),(Xas,Cb,YZf),(Xas,Cb,YZg),(Xas,Cb,YZh),(Xas,Cb,YZi),(Xas, Cb,YZj),(Xas,Cb,YZk),(Xas,Cb,YZl),(Xas,Cb,YZm),(Xas,Cb,YZn),(Xas,Cb,YZo),(Xas, Cb,YZp),(Xas,Cb,YZq),(Xas,Cb,YZr),(Xas,Cb,YZs),(Xas,Cb,YZt),(Xas,Cb,YZu), (Xas,Cb,YZv),(Xas,Cb,YZw),(Xas,Cb,YZx),(Xas,Cb,YZy),(Xas,Cb,YZz),(Xas,Cb,YZaa), (Xas,Cb,YZab),(Xas,Cb,YZac),(Xas,Cb,YZad),(Xas,Cb,YZae),(Xas,Cb,YZaf),(Xas, Cb,YZag),(Xas,Cb,YZah),(Xas,Cb,YZai),(Xas,Cb,YZaj),(Xas,Cb,YZak),(Xas,Cb,YZal), (Xas,Cb,YZam),(Xas,Cb,YZan),(Xas,Cb,YZao),(Xas,Cb,YZap),(Xas,Cb,YZaq),(Xas, Cb,YZar),(Xas,Cb,YZas),(Xas,Cb,YZat),(Xas,Cb,YZau),(Xas,Cb,YZav),(Xas,Cb, YZaw),(Xas,Cb,YZax),(Xas,Cb,YZay),(Xas,Cb,YZaz),(Xas,Cb,YZba),(Xas,Cb,YZbb), (Xas,Cb,YZbc),(Xas,Cb,YZbd),(Xas,Cb,YZbe),(Xas,Cb,YZbf),(Xas,Cb,YZbg),(Xas, Cb,YZbh),(Xas,Cb,YZbi),(Xas,Cb,YZbj),(Xas,Cb,YZbk),(Xas,Cb,YZbl),(Xas,Cb,YZbm), (Xas,Cb,YZbn),(Xas,Cb,YZbo),(Xas,Cb,YZbp),(Xas,Cb,YZbq),(Xas,Cb,YZbr),(Xas, Cb,YZbs),(Xas,Cb,YZbt),(Xas,Cb,YZbu),(Xas,Cb,YZbv),(Xas,Cb,YZbw),(Xas,Cb, YZbx),(Xas,Cb,YZby),(Xas,Cb,YZbz),(Xas,Cb,YZca),(Xas,Cb,YZcb),(Xas,Cb,YZcc), (Xas,Cb,YZcd),(Xas,Cb,YZce),(Xas,Cb,YZcf),(Xas,Cb,YZcg),(Xas,Cb,YZch),(Xas,Cb, YZci),(Xas,Cb,YZcj),(Xas,Cb,YZck),(Xas,Cb,YZcl),(Xas,Cb,YZcm),(Xas,Cb,YZcn), (Xas,Cb,YZco),(Xas,Cb,YZcp),(Xas,Cb,YZcq),(Xas,Cb,YZcr),(Xas,Cb,YZcs),(Xas, Cb,YZct),(Xas,Cb,YZcu),(Xas,Cb,YZcv),(Xas,Cb,YZcw),(Xas,Cb,YZcx),(Xas,Cb,YZcy), (Xas,Cb,YZcz),(Xas,Cb,YZda),(Xas,Cb,YZdb),(Xas,Cb,YZdc),(Xas,Cb,YZdd),(Xas, Cb,YZde),(Xas,Cb,YZdf),(Xas,Cb,YZdg),(Xas,Cb,YZdh),(Xas,Cb,YZdi),(Xas,Cb, YZdj),(Xas,Cb,YZdk),(Xas,Cb,YZdl),(Xas,Cb,YZdm),(Xas,Cb,YZdn),(Xas,Cb,YZdo), (Xas,Cb,YZdp),(Xas,Cb,YZdq),(Xas,Cb,YZdr),(Xas,Cb,YZds),(Xas,Cb,Yzdt),(Xas,Cb, YZdu),(Xas,Cb,YZdv),(Xas,Cb,YZdw),(Xas,Cb,YZdx),(Xas,Cb,YZdy),(Xas,Cb,YZdz), (Xas,Cb,YZea),(Xas,Cb,YZeb),(Xas,Cb,YZec),(Xas,Cb,YZed),(Xas,Cb,YZee),(Xas, Cb,YZef),(Xas,Cb,YZeg),(Xas,Cb,YZeh),(Xas,Cb,YZei),(Xas,Cb,YZej),(Xas,Cb,YZek), (Xas,Cb,YZel),(Xas,Cb,YZem),(Xas,Cb,YZen),(Xas,Cb,YZeo),(Xas,Cb,YZep),(Xas, Cb,YZeq),(Xas,Cb,YZer),(Xas,Cb,YZes),(Xas,Cb,YZet),(Xas,Cb,YZeu),(Xas,Cb,YZev), (Xas,Cb,YZew),(Xas,Cb,YZex),(Xas,Cb,YZey),(Xas,Cb,YZez),(Xas,Cb,YZfa),(Xas, Cb,YZfb),(Xas,Cb,YZfc),(Xas,Cb,YZfd),(Xas,Cb,YZfe),(Xas,Cb,YZff),(Xas,Cb, YZfg),(Xas,Cb,YZfh),(Xas,Cb,YZfi),(Xas,Cb,YZfj),(Xas,Cb,YZfk),(Xas,Cb,YZfl),(Xas, Cb,YZfm),(Xas,Cb,YZfn),(Xas,Cb,YZfo),(Xas,Cb,YZfp),(Xas,Cb,YZfq),(Xas,Cb, YZfr),(Xas,Cb,YZfs),(Xas,Cb,YZft),(Xas,Cb,YZfu),(Xas,Cb,YZfv),(Xas,Cb,YZfw),(Xas, Cb,YZfx),(Xas,Cb,YZfy),(Xas,Cb,YZfz),(Xas,Cb,YZga),(Xas,Cb,YZgb),(Xas,Cb, YZgc),(Xas,Cb,YZgd),(Xas,Cb,YZge),(Xas,Cb,YZgf),(Xas,Cb,YZgg),(Xas,Cb,YZgh), (Xas,Cb,YZgi),(Xas,Cb,YZgj),(Xas,Cb,YZgk),(Xas,Cb,YZgl),(Xas,Cb,YZgm),(Xas,Cb, YZgn),(Xas,Cb,YZgo),(Xas,Cb,YZgp),(Xas,Cb,YZgq),(Xas,Cb,YZgr),(Xas,Cb,YZgs), (Xas,Cb,YZgt),(Xas,Cc,YZa),(Xas,Cc,YZb),(Xas,Cc,YZc),(Xas,Cc,YZd),(Xas,Cc,YZe), (Xas,Cc,YZf),(Xas,Cc,YZg),(Xas,Cc,YZh),(Xas,Cc,YZi),(Xas,Cc,YZj),(Xas,Cc,YZk), (Xas,Cc,YZl),(Xas,Cc,YZm),(Xas,Cc,YZn),(Xas,Cc,YZo),(Xas,Cc,YZp),(Xas,Cc, YZq),(Xas,Cc,YZr),(Xas,Cc,YZs),(Xas,Cc,YZt),(Xas,Cc,YZu),(Xas,Cc,YZv),(Xas,Cc, YZw),(Xas,Cc,YZx),(Xas,Cc,YZy),(Xas,Cc,YZz),(Xas,Cc,YZaa),(Xas,Cc,YZab),(Xas, Cc,YZac),(Xas,Cc,YZad),(Xas,Cc,YZae),(Xas,Cc,YZaf),(Xas,Cc,YZag),(Xas,Cc,YZah), (Xas,Cc,YZai),(Xas,Cc,YZaj),(Xas,Cc,YZak),(Xas,Cc,YZal),(Xas,Cc,YZam),(Xas, Cc,YZan),(Xas,Cc,YZao),(Xas,Cc,YZap),(Xas,Cc,YZaq),(Xas,Cc,YZar),(Xas,Cc,YZas), (Xas,Cc,YZat),(Xas,Cc,YZau),(Xas,Cc,YZav),(Xas,Cc,YZaw),(Xas,Cc,YZax),(Xas, Cc,YZay),(Xas,Cc,YZaz),(Xas,Cc,YZba),(Xas,Cc,YZbb),(Xas,Cc,YZbc),(Xas,Cc,YZbd), (Xas,Cc,YZbe),(Xas,Cc,YZbf),(Xas,Cc,YZbg),(Xas,Cc,YZbh),(Xas,Cc,YZbi),(Xas, Cc,YZbj),(Xas,Cc,YZbk),(Xas,Cc,YZbl),(Xas,Cc,YZbm),(Xas,Cc,YZbn),(Xas,Cc,YZbo), (Xas,Cc,YZbp),(Xas,Cc,YZbq),(Xas,Cc,YZbr),(Xas,Cc,YZbs),(Xas,Cc,YZbt),(Xas, Cc,YZbu),(Xas,Cc,YZbv),(Xas,Cc,YZbw),(Xas,Cc,YZbx),(Xas,Cc,YZby),(Xas,Cc, YZbz),(Xas,Cc,YZca),(Xas,Cc,YZcb),(Xas,Cc,YZcc),(Xas,Cc,YZcd),(Xas,Cc,YZce),(Xas, Cc,YZcf),(Xas,Cc,YZcg),(Xas,Cc,YZch),(Xas,Cc,YZci),(Xas,Cc,YZcj),(Xas,Cc, YZck),(Xas,Cc,YZcl),(Xas,Cc,YZcm),(Xas,Cc,YZcn),(Xas,Cc,YZco),(Xas,Cc,YZcp), (Xas,Cc,YZcq),(Xas,Cc,YZcr),(Xas,Cc,YZcs),(Xas,Cc,YZct),(Xas,Cc,YZcu),(Xas,Cc, YZcv),(Xas,Cc,YZcw),(Xas,Cc,YZcx),(Xas,Cc,YZcy),(Xas,Cc,YZcz),(Xas,Cc,YZda), (Xas,Cc,YZdb),(Xas,Cc,YZdc),(Xas,Cc,YZdd),(Xas,Cc,YZde),(Xas,Cc,YZdf),(Xas,Cc, YZdg),(Xas,Cc,YZdh),(Xas,Cc,YZdi),(Xas,Cc,YZdj),(Xas,Cc,YZdk),(Xas,Cc,YZdl), (Xas,Cc,YZdm),(Xas,Cc,YZdn),(Xas,Cc,YZdo),(Xas,Cc,YZdp),(Xas,Cc,YZdq),(Xas, Cc,YZdr),(Xas,Cc,YZds),(Xas,Cc,Yzdt),(Xas,Cc,YZdu),(Xas,Cc,YZdv),(Xas,Cc,YZdw), (Xas,Cc,YZdx),(Xas,Cc,YZdy),(Xas,Cc,YZdz),(Xas,Cc,YZea),(Xas,Cc,YZeb),(Xas, Cc,YZec),(Xas,Cc,YZed),(Xas,Cc,YZee),(Xas,Cc,YZef),(Xas,Cc,YZeg),(Xas,Cc,YZeh), (Xas,Cc,YZei),(Xas,Cc,YZej),(Xas,Cc,YZek),(Xas,Cc,YZel),(Xas,Cc,YZem),(Xas, Cc,YZen),(Xas,Cc,YZeo),(Xas,Cc,YZep),(Xas,Cc,YZeq),(Xas,Cc,YZer),(Xas,Cc,YZes), (Xas,Cc,YZet),(Xas,Cc,YZeu),(Xas,Cc,YZev),(Xas,Cc,YZew),(Xas,Cc,YZex),(Xas, Cc,YZey),(Xas,Cc,YZez),(Xas,Cc,YZfa),(Xas,Cc,YZfb),(Xas,Cc,YZfc),(Xas,Cc,YZfd), (Xas,Cc,YZfe),(Xas,Cc,YZff),(Xas,Cc,YZfg),(Xas,Cc,YZfh),(Xas,Cc,YZfi),(Xas,Cc, YZfj),(Xas;Cc,YZfk),(Xas,Cc,YZfl),(Xas,Cc,YZfm),(Xas,Cc,YZfn),(Xas,Cc,YZfo),(Xas, Cc,YZfp),(Xas,Cc,YZfq),(Xas,Cc,YZfr),(Xas,Cc,YZfs),(Xas,Cc,YZft),(Xas,Cc,YZfu), (Xas,Cc,YZfv),(Xas,Cc,YZfw),(Xas,Cc,YZfx),(Xas,Cc,YZfy),(Xas,Cc,YZfz),(Xas, Cc,YZga),(Xas,Cc,YZgb),(Xas,Cc,YZgc),(Xas,Cc,YZgd),(Xas,Cc,YZge),(Xas,Cc,YZgf), (Xas,Cc,YZgg),(Xas,Cc,YZgh),(Xas,Cc,YZgi),(Xas,Cc,YZgj),(Xas,Cc,YZgk),(Xas, Cc,YZgl),(Xas,Cc,YZgm),(Xas,Cc,YZgn),(Xas,Cc,YZgo),(Xas,Cc,YZgp),(Xas,Cc, YZgq),(Xas,Cc,YZgr),(Xas,Cc,YZgs),(Xas,Cc,YZgt),(Xas,Cd,YZa),(Xas,Cd,YZb),(Xas, Cd,YZc),(Xas,Cd,YZd),(Xas,Cd,YZe),(Xas,Cd,YZf),(Xas,Cd,YZg),(Xas,Cd,YZh),(Xas, Cd,YZi),(Xas,Cd,YZj),(Xas,Cd,YZk),(Xas,Cd,YZl),(Xas,Cd,YZm),(Xas,Cd,YZn), (Xas,Cd,YZo),(Xas,Cd,YZp),(Xas,Cd,YZq),(Xas,Cd,YZr),(Xas,Cd,YZs),(Xas,Cd,YZt), (Xas,Cd,YZu),(Xas,Cd,YZv),(Xas,Cd,YZw),(Xas,Cd,YZx),(Xas,Cd,YZy),(Xas,Cd,YZz), (Xas,Cd,YZaa),(Xas,Cd,YZab),(Xas,Cd,YZac),(Xas,Cd,YZad),(Xas,Cd,YZae),(Xas, Cd,YZaf),(Xas,Cd,YZag),(Xas,Cd,YZah),(Xas,Cd,YZai),(Xas,Cd,YZaj),(Xas,Cd,YZak), (Xas,Cd,YZal),(Xas,Cd,YZam),(Xas,Cd,YZan),(Xas,Cd,YZao),(Xas,Cd,YZap),(Xas, Cd,YZaq),(Xas,Cd,YZar),(Xas,Cd,YZas),(Xas,Cd,YZat),(Xas,Cd,YZau),(Xas,Cd, YZav),(Xas,Cd,YZaw),(Xas,Cd,YZax),(Xas,Cd,YZay),(Xas,Cd,YZaz),(Xas,Cd,YZba), (Xas,Cd,YZbb),(Xas,Cd,YZbc),(Xas,Cd,YZbd),(Xas,Cd,YZbe),(Xas,Cd,YZbf),(Xas, Cd,YZbg),(Xas,Cd,YZbh),(Xas,Cd,YZbi),(Xas,Cd,YZbj),(Xas,Cd,YZbk),(Xas,Cd,YZbl), bl),(Xas,Cd,YZbm),(Xas,Cd,YZbn),(Xas,Cd,YZbo),(Xas,Cd,YZbp),(Xas,Cd,YZbq),(Xas, Cd,YZbr),(Xas,Cd,YZbs),(Xas,Cd,YZbt),(Xas,Cd,YZbu),(Xas,Cd,YZbv),(Xas,Cd, YZbw),(Xas,Cd,YZbx),(Xas,Cd,YZby),(Xas,Cd,YZbz),(Xas,Cd,YZca),(Xas,Cd,YZcb), (Xas,Cd,YZcc),(Xas,Cd,YZcd),(Xas,Cd,YZce),(Xas,Cd,YZcf),(Xas,Cd,YZcg),(Xas, Cd,YZch),(Xas,Cd,YZci),(Xas,Cd,YZcj),(Xas,Cd,YZck),(Xas,Cd,YZcl),(Xas,Cd,YZcm), (Xas,Cd,YZcn),(Xas,Cd,YZco),(Xas,Cd,YZcp),(Xas,Cd,YZcq),(Xas,Cd,YZcr),(Xas, Cd,YZcs),(Xas,Cd,YZct),(Xas,Cd,YZcu),(Xas,Cd,YZcv),(Xas,Cd,YZcw),(Xas,Cd,YZcx), (Xas,Cd,YZcy),(Xas,Cd,YZcz),(Xas,Cd,YZda),(Xas,Cd,YZdb),(Xas,Cd,YZdc),(Xas, Cd,YZdd),(Xas,Cd,YZde),(Xas,Cd,YZdf),(Xas,Cd,YZdg),(Xas,Cd,YZdh),(Xas,Cd, YZdi),(Xas,Cd,YZdj),(Xas,Cd,YZdk),(Xas,Cd,YZdl),(Xas,Cd,YZdm),(Xas,Cd,YZdn), (Xas,Cd,YZdo),(Xas,Cd,YZdp),(Xas,Cd,YZdq),(Xas,Cd,YZdr),(Xas,Cd,YZds),(Xas, Cd,YZdt),(Xas,Cd,YZdu),(Xas,Cd,YZdv),(Xas,Cd,YZdw),(Xas,Cd,YZdx),(Xas,Cd,YZdy), (Xas,Cd,YZdz),(Xas,Cd,YZea),(Xas,Cd,YZeb),(Xas,Cd,YZec),(Xas,Cd,YZed),(Xas, Cd,YZee),(Xas,Cd,YZef),(Xas,Cd,YZeg),(Xas,Cd,YZeh),(Xas,Cd,YZei),(Xas,Cd, YZej),(Xas,Cd,YZek),(Xas,Cd,YZel),(Xas,Cd,YZem),(Xas,Cd,YZen),(Xas,Cd,YZeo), (Xas,Cd,YZep),(Xas,Cd,YZeq),(Xas,Cd,YZer),(Xas,Cd,YZes),(Xas,Cd,YZet),(Xas,Cd, YZeu),(Xas,Cd,YZev),(Xas,Cd,YZew),(Xas,Cd,YZex),(Xas,Cd,YZey),(Xas,Cd,YZez), (Xas,Cd,YZfa),(Xas,Cd,YZfb),(Xas,Cd,YZfc),(Xas,Cd,YZfd),(Xas,Cd,YZfe),(Xas,Cd, YZff),(Xas,Cd,YZfg),(Xas,Cd,YZfh),(Xas,Cd,YZfi),(Xas,Cd,YZfj),(Xas,Cd,YZfk),(Xas, Cd,YZfl),(Xas,Cd,YZfm),(Xas,Cd,YZfn),(Xas,Cd,YZfo),(Xas,Cd,YZfp),(Xas,Cd, YZfq),(Xas,Cd,YZfr),(Xas,Cd,YZfs),(Xas,Cd,YZft),(Xas,Cd,YZfu),(Xas,Cd,YZfv),(Xas, Cd,YZfw),(Xas,Cd,YZfx),(Xas,Cd,YZfy),(Xas,Cd,YZfz),(Xas,Cd,YZga),(Xas,Cd, YZgb),(Xas,Cd,YZgc),(Xas,Cd,YZgd),(Xas,Cd,YZge),(Xas,Cd,YZgf),(Xas,Cd,YZgg), (Xas,Cd,YZgh),(Xas,Cd,YZgi),(Xas,Cd,YZgj),(Xas,Cd,YZgk),(Xas,Cd,YZgl),(Xas,Cd, YZgm),(Xas,Cd,YZgn),(Xas,Cd,YZgo),(Xas,Cd,YZgp),(Xas,Cd,YZgq),(Xas,Cd,YZgr), (Xas,Cd,YZgs),(Xas,Cd,YZgt),(Xas,Ce,YZa),(Xas,Ce,YZb),(Xas,Ce,YZc),(Xas,Ce, YZd),(Xas,Ce,YZe),(Xas,Ce,YZf),(Xas,Ce,YZg),(Xas,Ce,YZh),(Xas,Ce,YZi),(Xas,Ce, YZj),(Xas,Ce,YZk),(Xas,Ce,YZl),(Xas,Ce,YZm),(Xas,Ce,YZn),(Xas,Ce,YZo),(Xas,Ce, YZp),(Xas,Ce,YZq),(Xas,Ce,YZr),(Xas,Ce,YZs),(Xas,Ce,YZt),(Xas,Ce,YZu),(Xas,Ce, YZv),(Xas,Ce,YZw),(Xas,Ce,YZx),(Xas,Ce,YZy),(Xas,Ce,YZz),(Xas,Ce,YZaa),(Xas, Ce,YZab),(Xas,Ce,YZac),(Xas,Ce,YZad),(Xas,Ce,YZae),(Xas,Ce,YZaf),(Xas,Ce,YZag), (Xas,Ce,YZah),(Xas,Ce,YZai),(Xas,Ce,Yaj),(Xas,Ce,YZak),(Xas,Ce,YZal),(Xas, Ce,YZam),(Xas,Ce,YZan),(Xas,Ce,YZao),(Xas,Ce,YZap),(Xas,Ce,YZaq),(Xas,Ce,YZar), (Xas,Ce,YZas),(Xas,Ce,YZat),(Xas,Ce,YZau),(Xas,Ce,YZav),(Xas,Ce,YZaw),(Xas, Ce,YZax),(Xas,Ce,YZay),(Xas,Ce,YZaz),(Xas,Ce,YZba),(Xas,Ce,YZbb),(Xas,Ce,YZbc), (Xas,Ce,YZbd),(Xas,Ce,YZbe),(Xas,Ce,YZbf),(Xas,Ce,YZbg),(Xas,Ce,YZbh),(Xas, Ce,YZbi),(Xas,Ce,YZbj),(Xas,Ce,YZbk),(Xas,Ce,YZbl),(Xas,Ce,YZbm),(Xas,Ce,YZbn), (Xas,Ce,YZbo),(Xas,Ce,YZbp),(Xas,Ce,YZbq),(Xas,Ce,YZbr),(Xas,Ce,YZbs),(Xas, Ce,YZbt),(Xas,Ce,YZbu),(Xas,Ce,YZbv),(Xas,Ce,YZbw),(Xas,Ce,YZbx),(Xas,Ce, YZby),(Xas,Ce,YZbz),(Xas,Ce,YZca),(Xas,Ce,YZcb),(Xas,Ce,YZcc),(Xas,Ce,YZcd), (Xas,Ce,YZce),(Xas,Ce,YZcf),(Xas,Ce,YZcg),(Xas,Ce,YZch),(Xas,Ce,YZci),(Xas,Ce, YZcj),(Xas,Ce,YZck),(Xas,Ce,YZcl),(Xas,Ce,YZcm),(Xas,Ce,YZcn),(Xas,Ce,YZco),(Xas, Ce,YZcp),(Xas,Ce,YZcq),(Xas,Ce,YZcr),(Xas,Ce,YZcs),(Xas,Ce,YZct),(Xas,Ce, YZcu),(Xas,Ce,YZcv),(Xas,Ce,YZcw),(Xas,Ce,YZcx),(Xas,Ce,YZcy),(Xas,Ce,YZcz), (Xas,Ce,YZda),(Xas,Ce,YZdb),(Xas,Ce,YZdc),(Xas,Ce,YZdd),(Xas,Ce,YZde),(Xas,Ce, YZdf),(Xas,Ce,YZdg),(Xas,Ce,YZdh),(Xas,Ce,YZdi),(Xas,Ce,YZdj),(Xas,Ce,YZdk), (Xas,Ce,YZdl),(Xas,Ce,YZdm),(Xas,Ce,YZdn),(Xas,Ce,YZdo),(Xas,Ce,YZdp),(Xas, Ce,YZdq),(Xas,Ce,YZdr),(Xas,Ce,YZds),(Xas,Ce,YZdt),(Xas,Ce,YZdu),(Xas,Ce,YZdv), (Xas,Ce,YZdw),(Xas,Ce,YZdx),(Xas,Ce,YZdy),(Xas,Ce,YZdz),(Xas,Ce,YZea),(Xas, Ce,YZeb),(Xas,Ce,YZec),(Xas,Ce,YZed),(Xas,Ce,YZee),(Xas,Ce,YZef),(Xas,Ce,YZeg), (Xas,Ce,YZeh),(Xas,Ce,YZei),(Xas,Ce,YZej),(Xas,Ce,YZek),(Xas,Ce,YZel),(Xas, Ce,YZem),(Xas,Ce,YZen),(Xas,Ce,YZeo),(Xas,Ce,YZep),(Xas,Ce,YZeq),(Xas,Ce,YZer), (Xas,Ce,YZes),(Xas,Ce,YZet),(Xas,Ce,YZeu),(Xas,Ce,YZev),(Xas,Ce,YZew),(Xas, Ce,YZex),(Xas,Ce,YZey),(Xas,Ce,YZez),(Xas,Ce,YZfa),(Xas,Ce,YZfb),(Xas,Ce,YZfc), (Xas,Ce,YZfd),(Xas,Ce,YZfe),(Xas,Ce,YZff),(Xas,Ce,YZfg),(Xas,Ce,YZfh),(Xas,Ce, YZfi),(Xas,Ce,YZfj),(Xas,Ce,YZfk),(Xas,Ce,YZfl),(Xas,Ce,YZfm),(Xas,Ce,YZfn),(Xas, Ce,YZfo),(Xas,Ce,YZfp),(Xas,Ce,YZfq),(Xas,Ce,YZfr),(Xas,Ce,YZfs),(Xas,Ce,YZft), (Xas,Ce,YZfu),(Xas,Ce,YZfv),(Xas,Ce,YZfw),(Xas,Ce,YZfx),(Xas,Ce,YZfy),(Xas, Ce,YZfz),(Xas,Ce,YZga),(Xas,Ce,YZgb),(Xas,Ce,YZgc),(Xas,Ce,YZgd),(Xas,Ce,YZge), (Xas,Ce,YZgf),(Xas,Ce,YZgg),(Xas,Ce,YZgh),(Xas,Ce,YZgi),(Xas,Ce,YZgj),(Xas, Ce,YZgk),(Xas,Ce,YZgl),(Xas,Ce,YZgm),(Xas,Ce,YZgn),(Xas,Ce,YZgo),(Xas,Ce, YZgp),(Xas,Ce,YZgq),(Xas,Ce,YZgr),(Xas,Ce,YZgs),(Xas,Ce,YZgt),(Xas,Cf,YZa),(Xas, Cf,YZb),(Xas,Cf,YZc),(Xas,Cf,YZd),(Xas,Cf,YZe),(Xas,Cf,YZf),(Xas,Cf,YZg),(Xas, Cf,YZh),(Xas,Cf,YZi),(Xas,Cf,YZj),(Xas,Cf,YZk),(Xas,Cf,YZl),(Xas,Cf,YZm),(Xas, Cf,YZn),(Xas,Cf,YZo),(Xas,Cf,YZp),(Xas,Cf,YZq),(Xas,Cf,YZr),(Xas,Cf,YZs),(Xas, Cf,YZt),(Xas,Cf,YZu),(Xas,Cf,YZv),(Xas,Cf,YZw),(Xas,Cf,YZx),(Xas,Cf,YZy),(Xas, Cf,YZz),(Xas,Cf,YZaa),(Xas,Cf,YZab),(Xas,Cf,YZac),(Xas,Cf,YZad),(Xas,Cf,YZae), (Xas,Cf,YZaf),(Xas,Cf,YZag),(Xas,Cf,YZah),(Xas,Cf,YZai),(Xas,Cf,YZaj),(Xas,Cf,YZak), (Xas,Cf,YZal),(Xas,Cf,YZam),(Xas,Cf,YZan),(Xas,Cf,YZao),(Xas,Cf,YZap),(Xas, Cf,YZaq),(Xas,Cf,YZar),(Xas,Cf,YZas),(Xas,Cf,YZat),(Xas,Cf,YZau),(Xas,Cf,YZav), (Xas,Cf,YZaw),(Xas,Cf,YZax),(Xas,Cf,YZay),(Xas,Cf,YZaz),(Xas,Cf,YZba),(Xas,Cf, YZbb),(Xas,Cf,YZbc),(Xas,Cf,YZbd),(Xas,Cf,YZbe),(Xas,Cf,YZbf),(Xas,Cf,YZbg), (Xas,Cf,YZbh),(Xas,Cf,YZbi),(Xas,Cf,YZbj),(Xas,Cf,YZbk),(Xas,Cf,YZbl),(Xas,Cf,YZbm), (Xas,Cf,YZbn),(Xas,Cf,YZbo),(Xas,Cf,YZbp),(Xas,Cf,YZbq),(Xas,Cf,YZbr),(Xas, Cf,YZbs),(Xas,Cf,YZbt),(Xas,Cf,YZbu),(Xas,Cf,YZbv),(Xas,Cf,YZbw),(Xas,Cf,YZbx), (Xas,Cf,YZby),(Xas,Cf,YZbz),(Xas,Cf,YZca),(Xas,Cf,YZcb),(Xas,Cf,YZcc),(Xas, Cf,YZcd),(Xas,Cf,YZce),(Xas,Cf,YZcf),(Xas,Cf,YZcg),(Xas,Cf,YZch),(Xas,Cf,YZci), (Xas,Cf,YZcj),(Xas,Cf,YZck),(Xas,Cf,YZcl),(Xas,Cf,YZcm),(Xas,Cf,YZcn),(Xas,Cf, YZco),(Xas,Cf,YZcp),(Xas,Cf,YZcq),(Xas,Cf,YZcr),(Xas,Cf,YZcs),(Xas,Cf,YZct),(Xas, Cf,YZcu),(Xas,Cf,YZcv),(Xas,Cf,YZcw),(Xas,Cf,YZcx),(Xas,Cf,YZcy),(Xas,Cf,YZcz), (Xas,Cf,YZda),(Xas,Cf,YZdb),(Xas,Cf,YZdc),(Xas,Cf,YZdd),(Xas,Cf,YZde),(Xas, Cf,YZdf),(Xas,Cf,YZdg),(Xas,Cf,YZdh),(Xas,Cf,YZdi),(Xas,Cf,YZdj),(Xas,Cf,YZdk), (Xas,Cf,YZdl),(Xas,Cf,YZdm),(Xas,Cf,YZdn),(Xas,Cf,YZdo),(Xas,Cf,YZdp),(Xas, Cf,YZdq),(Xas,Cf,YZdr),(Xas,Cf,YZds),(Xas,Cf,YZdt),(Xas,Cf,YZdu),(Xas,Cf,YZdv), (Xas,Cf,YZdw),(Xas,Cf,YZdx),(Xas,Cf,YZdy),(Xas,Cf,YZdz),(Xas,Cf,YZea),(Xas,Cf, YZeb),(Xas,Cf,YZec),(Xas,Cf,YZed),(Xas,Cf,YZee),(Xas,Cf,YZef),(Xas,Cf,YZeg),(Xas, Cf,YZeh),(Xas,Cf,YZei),(Xas,Cf,YZej),(Xas,Cf,YZek),(Xas,Cf,YZel),(Xas,Cf,YZem), (Xas,Cf,YZen),(Xas,Cf,YZeo),(Xas,Cf,YZep),(Xas,Cf,YZeq),(Xas,Cf,YZer),(Xas, Cf,YZes),(Xas,Cf,YZet),(Xas,Cf,YZeu),(Xas,Cf,YZev),(Xas,Cf,YZew),(Xas,Cf,YZex), (Xas,Cf,YZey),(Xas,Cf,YZez),(Xas,Cf,YZfa),(Xas,Cf,YZfb),(Xas,Cf,YZfc),(Xas,Cf, YZfd),(Xas,Cf,YZfe),(Xas,Cf,YZff),(Xas,Cf,YZfg),(Xas,Cf,YZfh),(Xas,Cf,YZfi),(Xas, Cf,YZfj),(Xas,Cf,YZfk),(Xas,Cf,YZfl),(Xas,Cf,YZfm),(Xas,Cf,YZfn),(Xas,Cf,YZfo), (Xas,Cf,YZfp),(Xas,Cf,YZfq),(Xas,Cf,YZfr),(Xas,Cf,YZfs),(Xas,Cf,YZft),(Xas,Cf,YZfu), (Xas,Cf,YZfv),(Xas,Cf,YZfw),(Xas,Cf,YZfx),(Xas,Cf,YZfy),(Xas,Cf,YZfz),(Xas, Cf,YZga),(Xas,Cf,YZgb),(Xas,Cf,YZgc),(Xas,Cf,YZgd),(Xas,Cf,YZge),(Xas,Cf,YZgf), (Xas,Cf,YZgg),(Xas,Cf,YZgh),(Xas,Cf,YZgi),(Xas,Cf,YZgj),(Xas,Cf,YZgk),(Xas,Cf, YZgl),(Xas,Cf,YZgm),(Xas,Cf,YZgn),(Xas,Cf,YZgo),(Xas,Cf,YZgp),(Xas,Cf,YZgq), (Xas,Cf,YZgr),(Xas,Cf,YZgs),(Xas,Cf,YZgt), (Xat,Ca,YZa),(Xat,Ca,YZb),(Xat,Ca,YZc),(Xat,Ca,YZd),(Xat,Ca,YZe),(Xat,Ca,YZf),(Xat, Ca,YZg),(Xat,Ca,YZh),(Xat,Ca,YZi),(Xat,Ca,YZj),(Xat,Ca,YZk),(Xat,Ca,YZl),(Xat, Ca,YZm),(Xat,Ca,YZn),(Xat,Ca,YZo),(Xat,Ca,YZp),(Xat,Ca,YZq),(Xat,Ca,YZr),(Xat, Ca,YZs),(Xat,Ca,YZt),(Xat,Ca,YZu),(Xat,Ca,YZv),(Xat,Ca,YZw),(Xat,Ca,YZx),(Xat, Ca,YZy),(Xat,Ca,YZz),(Xat,Ca,YZaa),(Xat,Ca,YZab),(Xat,Ca,YZac),(Xat,Ca,YZad), (Xat,Ca,YZae),(Xat,Ca,YZaf),(Xat,Ca,YZag),(Xat,Ca,YZah),(Xat,Ca,YZai),(Xat,Ca, YZaj),(Xat,Ca,YZak),(Xat,Ca,YZal),(Xat,Ca,YZam),(Xat,Ca,YZan),(Xat,Ca,YZao),(Xat, Ca,YZap),(Xat,Ca,YZaq),(Xat,Ca,YZar),(Xat,Ca,YZas),(Xat,Ca,YZat),(Xat,Ca,YZau), (Xat,Ca,YZav),(Xat,Ca,YZaw),(Xat,Ca,YZax),(Xat,Ca,YZay),(Xat,Ca,YZaz),(Xat, Ca,YZba),(Xat,Ca,YZbb),(Xat,Ca,YZbc),(Xat,Ca,YZbd),(Xat,Ca,YZbe),(Xat,Ca,YZbf), (Xat,Ca,YZbg),(Xat,Ca,YZbh),(Xat,Ca,YZbi),(Xat,Ca,YZbj),(Xat,Ca,YZbk),(Xat,Ca, YZbl),(Xat,Ca,YZbm),(Xat,Ca,YZbn),(Xat,Ca,YZbo),(Xat,Ca,YZbp),(Xat,Ca,YZbq), (Xat,Ca,YZbr),(Xat,Ca,YZbs),(Xat,Ca,YZbt),(Xat,Ca,YZbu),(Xat,Ca,YZbv),(Xat,Ca, YZbw),(Xat,Ca,YZbx),(Xat,Ca,YZby),(Xat,Ca,YZbz),(Xat,Ca,YZca),(Xat,Ca,YZcb),(Xat, Ca,YZcc),(Xat,Ca,YZcd),(Xat,Ca,YZce),(Xat,Ca,YZcf),(Xat,Ca,YZcg),(Xat,Ca,YZch), (Xat,Ca,YZci),(Xat,Ca,YZcj),(Xat,Ca,YZck),(Xat,Ca,YZcl),(Xat,Ca,YZcm),(Xat, Ca,YZcn),(Xat,Ca,YZco),(Xat,Ca,YZcp),(Xat,Ca,YZcq),(Xat,Ca,YZcr),(Xat,Ca,YZcs), (Xat,Ca,YZct),(Xat,Ca,YZcu),(Xat,Ca,YZcv),(Xat,Ca,YZcw),(Xat,Ca,YZcx),(Xat,Ca, YZcy),(Xat,Ca,YZcz),(Xat,Ca,YZda),(Xat,Ca,YZdb),(Xat,Ca,YZdc),(Xat,Ca,YZdd),(Xat, Ca,YZde),(Xat,Ca,YZdf),(Xat,Ca,YZdg),(Xat,Ca,YZdh),(Xat,Ca,YZdi),(Xat,Ca,YZdj), (Xat,Ca,YZdk),(Xat,Ca,YZdl),(Xat,Ca,YZdm),(Xat,Ca,YZdn),(Xat,Ca,YZdo),(Xat, Ca,YZdp),(Xat,Ca,YZdq),(Xat,Ca,YZdr),(Xat,Ca,YZds),(Xat,Ca,YZdt),(Xat,Ca,YZdu), (Xat,Ca,YZdv),(Xat,Ca,YZdw),(Xat,Ca,YZdx),(Xat,Ca,YZdy),(Xat,Ca,YZdz),(Xat, Ca,YZea),(Xat,Ca,YZeb),(Xat,Ca,YZec),(Xat,Ca,YZed),(Xat,Ca,YZee),(Xat,Ca,YZef), (Xat,Ca,YZeg),(Xat,Ca,YZeh),(Xat,Ca,YZei),(Xat,Ca,YZej),(Xat,Ca,YZek),(Xat,Ca, YZel),(Xat,Ca,YZem),(Xat,Ca,YZen),(Xat,Ca,YZeo),(Xat,Ca,YZep),(Xat,Ca,YZeq),(Xat, Ca,YZer),(Xat,Ca,YZes),(Xat,Ca,YZet),(Xat,Ca,YZeu),(Xat,Ca,YZev),(Xat,Ca,YZew), (Xat,Ca,YZex),(Xat,Ca,YZey),(Xat,Ca,YZez),(Xat,Ca,YZfa),(Xat,Ca,YZfb),(Xat, Ca,YZfc),(Xat,Ca,YZfd),(Xat,Ca,YZfe),(Xat,Ca,YZff),(Xat,Ca,YZfg),(Xat,Ca,YZfh), (Xat,Ca,YZfi),(Xat,Ca,YZfj),(Xat,Ca,YZfk),(Xat,Ca,YZfl),(Xat,Ca,YZfm),(Xat,Ca,YZfn), (Xat,Ca,YZfo),(Xat,Ca,YZfp,(Xat,Ca,YZfq),(Xat,Ca,YZfr),(Xat,Ca,YZfs),(Xat, Ca,YZft),(Xat,Ca,YZfu),(Xat,Ca,YZfv),(Xat,Ca,YZfw),(Xat,Ca,YZfx),(Xat,Ca,YZfy), (Xat,Ca,YZfz),(Xat,Ca,YZga),(Xat,Ca,YZgb),(Xat,Ca,YZgc),(Xat,Ca,YZgd),(Xat,Ca, YZge),(Xat,Ca,YZgf),(Xat,Ca,YZgg),(Xat,Ca,YZgh),(Xat,Ca,YZgi),(Xat,Ca,YZgj),(Xat, Ca,YZgk),(Xat,Ca,YZgl),(Xat,Ca,YZgm),(Xat,Ca,YZgn),(Xat,Ca,YZgo),(Xat,Ca,YZgp), (Xat,Ca,YZgq),(Xat,Ca,YZgr),(Xat,Ca,YZgs),(Xat,Ca,YZgt),(Xat,Cb,YZa),(Xat, Cb,YZb),(Xat,Cb,YZc),(Xat,Cb,YZd),(Xat,Cb,YZe),(Xat,Cb,YZf),(Xat,Cb,YZg),(Xat, Cb,YZh),(Xat,Cb,YZi),(Xat,Cb,YZj),(Xat,Cb,YZk),(Xat,Cb,YZl),(Xat,Cb,YZm),(Xat, Cb,YZn),(Xat,Cb,YZo),(Xat,Cb,YZp),(Xat,Cb,YZq),(Xat,Cb,YZr),(Xat,Cb,YZs),(Xat, Cb,YZt),(Xat,Cb,YZu),(Xat,Cb,YZv),(Xat,Cb,YZw),(Xat,Cb,YZx),(Xat,Cb,YZy),(Xat, Cb,YZz),(Xat,Cb,YZaa),(Xat,Cb,YZab),(Xat,Cb,YZac),(Xat,Cb,YZad),(Xat,Cb,YZae), (Xat,Cb,YZaf),(Xat,Cb,YZag),(Xat,Cb,YZah),(Xat,Cb,YZai),(Xat,Cb,YZaj),(Xat,Cb, YZak),(Xat,Cb,YZal),(Xat,Cb,YZam),(Xat,Cb,YZan),(Xat,Cb,YZao),(Xat,Cb,YZap),(Xat, Cb,YZaq),(Xat,Cb,YZar),(Xat,Cb,YZas),(Xat,Cb,YZat),(Xat,Cb,YZau),(Xat,Cb,YZav), (Xat,Cb,YZaw),(Xat,Cb,YZax),(Xat,Cb,YZay),(Xat,Cb,YZaz),(Xat,Cb,YZba),(Xat, Cb,YZbb),(Xat,Cb,YZbc),(Xat,Cb,YZbd),(Xat,Cb,YZbe),(Xat,Cb,YZbf),(Xat,Cb,YZbg), (Xat,Cb,YZbh),(Xat,Cb,YZbi),(Xat,Cb,YZbj),(Xat,Cb,YZbk),(Xat,Cb,YZbl),(Xat, Cb,YZbm),(Xat,Cb,YZbn),(Xat,Cb,YZbo),(Xat,Cb,YZbp),(Xat,Cb,YZbq),(Xat,Cb,YZbr), (Xat,Cb,YZbs),(Xat,Cb,YZbt),(Xat,Cb,YZbu),(Xat,Cb,YZbv),(Xat,Cb,YZbw),(Xat, Cb,YZbx),(Xat,Cb,YZby),(Xat,Cb,YZbz),(Xat,Cb,YZca),(Xat,Cb,YZcb),(Xat,Cb,YZcc), (Xat,Cb,YZcd),(Xat,Cb,YZce),(Xat,Cb,YZcf),(Xat,Cb,YZcg),(Xat,Cb,YZch),(Xat, Cb,YZei),(Xat,Cb,YZcj),(Xat,Cb,YZck),(Xat,Cb,YZcl),(Xat,Cb,YZcm),(Xat,Cb,YZcn), (Xat,Cb,YZco),(Xat,Cb,YZcp),(Xat,Cb,YZcq),(Xat,Cb,YZcr),(Xat,Cb,YZcs),(Xat,Cb, YZct),(Xat,Cb,YZcu),(Xat,Cb,YZcv),(Xat,Cb,YZcw),(Xat,Cb,YZcx),(Xat,Cb,YZcy), (Xat,Cb,YZcz),(Xat,Cb,YZda),(Xat,Cb,YZdb),(Xat,Cb,YZdc),(Xat,Cb,YZdd),(Xat, Cb,YZde),(Xat,Cb,YZdf),(Xat,Cb,YZdg),(Xat,Cb,YZdh),(Xat,Cb,YZdi),(Xat,Cb,YZdj), (Xat,Cb,YZdk),(Xat,Cb,YZdl),(Xat,Cb,YZdm),(Xat,Cb,YZdn),(Xat,Cb,YZdo),(Xat, Cb,YZdp),(Xat,Cb,YZdq),(Xat,Cb,YZdr),(Xat,Cb,YZds),(Xat,Cb,YZdt),(Xat,Cb,YZdu), (Xat,Cb,YZdv),(Xat,Cb,YZdw),(Xat,Cb,YZdx),(Xat,Cb,YZdy),(Xat,Cb,YZdz),(Xat, Cb,YZea),(Xat,Cb,YZeb),(Xat,Cb,YZec),(Xat,Cb,YZed),(Xat,Cb,YZee),(Xat,Cb,YZef), (Xat,Cb,YZeg),(Xat,Cb,YZeh),(Xat,Cb,YZei),(Xat,Cb,YZej),(Xat,Cb,YZek),(Xat,Cb, YZel),(Xat,Cb,YZem),(Xat,Cb,YZen),(Xat,Cb,YZeo),(Xat,Cb,YZep),(Xat,Cb,YZeq), (Xat,Cb,YZer),(Xat,Cb,YZes),(Xat,Cb,YZet),(Xat,Cb,YZeu),(Xat,Cb,YZev),(Xat,Cb, YZew),(Xat,Cb,YZex),(Xat,Cb,YZey),(Xat,Cb,YZez),(Xat,Cb,YZfa),(Xat,Cb,YZfb),(Xat, Cb,YZfc),(Xat,Cb,YZfd),(Xat,Cb,YZfe),(Xat,Cb,YZff),(Xat,Cb,YZfg),(Xat,Cb,YZfh), (Xat,Cb,YZfi),(Xat,Cb,YZfj),(Xat,Cb,YZfk),(Xat,Cb,YZfl),(Xat,Cb,YZfm),(Xat, Cb,YZfn),(Xat,Cb,YZfo),(Xat,Cb,YZfp),(Xat,Cb,YZfq),(Xat,Cb,YZfr),(Xat,Cb,YZfs), (Xat,Cb,YZft),(Xat,Cb,YZfu),(Xat,Cb,YZfv),(Xat,Cb,YZfw),(Xat,Cb,YZfx),(Xat,Cb, YZfy),(Xat,Cb,YZfz),(Xat,Cb,YZga),(Xat,Cb,YZgb),(Xat,Cb,YZgc),(Xat,Cb,YZgd),(Xat, Cb,YZge),(Xat,Cb,YZgf),(Xat,Cb,YZgg),(Xat,Cb,YZgh),(Xat,Cb,YZgi),(Xat,Cb, YZgj),(Xat,Cb,YZgk),(Xat,Cb,YZgl),(Xat,Cb,YZgm),(Xat,Cb,YZgn),(Xat,Cb,YZgo),(Xat, Cb,YZgp),(Xat,Cb,YZgq),(Xat,Cb,YZgr),(Xat,Cb,YZgs),(Xat,Cb,YZgt),(Xat,Cc, YZa),(Xat,Cc,YZb),(Xat,Cc,YZc),(Xat,Cc,YZd),(Xat,Cc,YZe),(Xat,Cc,YZf),(Xat,Cc, YZg),(Xat,Cc,YZh),(Xat,Cc,YZi),(Xat,Cc,YZj),(Xat,Cc,YZk),(Xat,Cc,YZl),(Xat,Cc,YZm), (Xat,Cc,YZn),(Xat,Cc,YZo),(Xat,Cc,YZp),(Xat,Cc,YZq),(Xat,Cc,YZr),(Xat,Cc,YZs), (Xat,Cc,YZt),(Xat,Cc,YZu),(Xat,Cc,YZv),(Xat,Cc,YZw),(Xat,Cc,YZx),(Xat,Cc,YZy), (Xat,Cc,YZz),(Xat,Cc,YZaa),(Xat,Cc,YZab),(Xat,Cc,YZac),(Xat,Cc,YZad),(Xat, Cc,YZae),(Xat,Cc,YZaf),(Xat,Cc,YZag),(Xat,Cc,YZah),(Xat,Cc,YZai),(Xat,Cc,YZaj), (Xat,Cc,YZak),(Xat,Cc,YZal),(Xat,Cc,YZam),(Xat,Cc,YZan),(Xat,Cc,YZao),(Xat,Cc, YZap),(Xat,Cc,YZaq),(Xat,Cc,YZar),(Xat,Cc,YZas),(Xat,Cc,YZat),(Xat,Cc,YZau),(Xat, Cc,YZav),(Xat,Cc,YZaw),(Xat,Cc,YZax),(Xat,Cc,YZay),(Xat,Cc,YZaz),(Xat,Cc,YZba), (Xat,Cc,YZbb),(Xat,Cc,YZbc),(Xat,Cc,YZbd),(Xat,Cc,YZbe),(Xat,Cc,YZbf),(Xat, Cc,YZbg),(Xat,Cc,YZbh),(Xat,Cc,YZbi),(Xat,Cc,YZbj),(Xat,Cc,YZbk),(Xat,Cc,YZbl), (Xat,Cc,YZbm),(Xat,Cc,YZbn),(Xat,Cc,YZbo),(Xat,Cc,YZbp),(Xat,Cc,YZbq),(Xat, Cc,YZbr),(Xat,Cc,YZbs),(Xat,Cc,YZbt),(Xat,Cc,YZbu),(Xat,Cc,YZbv),(Xat,Cc,YZbw), (Xat,Cc,YZbx),(Xat,Cc,YZby),(Xat,Cc,YZbz),(Xat,Cc,YZca),(Xat,Cc,YZcb),(Xat,Cc, YZcc),(Xat,Cc,YZcd),(Xat,Cc,YZce),(Xat,Cc,YZcf),(Xat,Cc,YZcg),(Xat,Cc,YZch),(Xat, Cc,YZci),(Xat,Cc,YZcj),(Xat,Cc,YZck),(Xat,Cc,YZcl),(Xat,Cc,YZcm),(Xat,Cc,YZcn), (Xat,Cc,YZco),(Xat,Cc,YZcp),(Xat,Cc,YZcq),(Xat,Cc,YZcr),(Xat,Cc,YZcs),(Xat, Cc,YZct),(Xat,Cc,YZcu),(Xat,Cc,YZcv),(Xat,Cc,YZcw),(Xat,Cc,YZcx),(Xat,Cc,YZcy), (Xat,Cc,YZcz),(Xat,Cc,YZda),(Xat,Cc,YZdb),(Xat,Cc,YZdc),(Xat,Cc,YZdd),(Xat, Cc,YZde),(Xat,Cc,YZdf),(Xat,Cc,YZdg),(Xat,Cc,YZdh),(Xat,Cc,YZdi),(Xat,Cc,YZdj), (Xat,Cc,YZdk),(Xat,Cc,YZdl),(Xat,Cc,YZdm),(Xat,Cc,YZdn),(Xat,Cc,YZdo),(Xat,Cc, YZdp),(Xat,Cc,YZdq),(Xat,Cc,YZdr),(Xat,Cc,YZds),(Xat,Cc,YZdt),(Xat,Cc,YZdu),(Xat, Cc,YZdv),(Xat,Cc,YZdw),(Xat,Cc,YZdx),(Xat,Cc,YZdy),(Xat,Cc,YZdz),(Xat,Cc, YZea),(Xat,Cc,YZeb),(Xat,Cc,YZec),(Xat,Cc,YZed),(Xat,Cc,YZee),(Xat,Cc,YZef),(Xat, Cc,YZeg),(Xat,Cc,YZeh),(Xat,Cc,YZei),(Xat,Cc,YZej),(Xat,Cc,YZek),(Xat,Cc,YZel), (Xat,Cc,YZem),(Xat,Cc,YZen),(Xat,Cc,YZeo),(Xat,Cc,YZep),(Xat,Cc,YZeq),(Xat, Cc,YZer),(Xat,Cc,YZes),(Xat,Cc,YZet),(Xat,Cc,YZeu),(Xat,Cc,YZev),(Xat,Cc,YZew), (Xat,Cc,YZex),(Xat,Cc,YZey),(Xat,Cc,YZez),(Xat,Cc,YZfa),(Xat,Cc,YZfb),(Xat,Cc, YZfc),(Xat,Cc,YZfd),(Xat,Cc,YZfe),(Xat,Cc,YZff),(Xat,Cc,YZfg),(Xat,Cc,YZfh),(Xat, Cc,YZfi),(Xat,Cc,YZfj),(Xat,Cc,YZfk),(Xat,Cc,YZfl),(Xat,Cc,YZfm),(Xat,Cc,YZfn),(Xat, Cc,YZfo),(Xat,Cc,YZfp),(Xat,Cc,YZfq),(Xat,Cc,YZfr),(Xat,Cc,YZfs),(Xat,Cc,YZft), (Xat,Cc,YZfu),(Xat,Cc,YZfv),(Xat,Cc,YZfw),(Xat,Cc,YZfx),(Xat,Cc,YZfy),(Xat,Cc, YZfz),(Xat,Cc,YZga),(Xat,Cc,YZgb),(Xat,Cc,YZgc),(Xat,Cc,YZgd),(Xat,Cc,YZge), (Xat,Cc,YZgf),(Xat,Cc,YZgg),(Xat,Cc,YZgh),(Xat,Cc,YZgi),(Xat,Cc,YZgj),(Xat,Cc, YZgk),(Xat,Cc,YZgl),(Xat,Cc,YZgm),(Xat,Cc,YZgn),(Xat,Cc,YZgo),(Xat,Cc,YZgp),(Xat, Cc,YZgq),(Xat,Cc,YZgr),(Xat,Cc,YZgs),(Xat,Cc,YZgt),(Xat,Cd,YZa),(Xat,Cd,YZb), (Xat,Cd,YZc),(Xat,Cd,YZd),(Xat,Cd,YZe),(Xat,Cd,YZf),(Xat,Cd,YZg),(Xat,Cd,YZh), (Xat,Cd,YZi),(Xat,Cd,YZj),(Xat,Cd,YZk),(Xat,Cd,YZl),(Xat,Cd,YZm),(Xat,Cd,YZn), (Xat,Cd,YZo),(Xat,Cd,YZp),(Xat,Cd,YZq),(Xat,Cd,YZr),(Xat,Cd,YZs),(Xat,Cd,YZt), (Xat,Cd,YZu),(Xat,Cd,YZv),(Xat,Cd,YZw),(Xat,Cd,YZx),(Xat,Cd,YZy),(Xat,Cd,YZz), (Xat,Cd,YZaa),(Xat,Cd,YZab),(Xat,Cd,YZac),(Xat,Cd,YZad),(Xat,Cd,YZae),(Xat, Cd,YZaf),(Xat,Cd,YZag),(Xat,Cd,YZah),(Xat,Cd,YZai),(Xat,Cd,YZaj),(Xat,Cd,YZak), (Xat,Cd,YZal),(Xat,Cd,YZam),(Xat,Cd,YZan),(Xat,Cd,YZao),(Xat,Cd,YZap),(Xat,Cd, YZaq),(Xat,Cd,YZar),(Xat,Cd,YZas),(Xat,Cd,YZat),(Xat,Cd,YZau),(Xat,Cd,YZav),(Xat, Cd,YZaw),(Xat,Cd,YZax),(Xat,Cd,YZay),(Xat,Cd,YZaz),(Xat,Cd,YZba),(Xat,Cd, YZbb),(Xat,Cd,YZbc),(Xat,Cd,YZbd),(Xat,Cd,YZbe),(Xat,Cd,YZbf),(Xat,Cd,YZbg),(Xat, Cd,YZbh),(Xat,Cd,YZbi),(Xat,Cd,YZbj),(Xat,Cd,YZbk),(Xat,Cd,YZbl),(Xat,Cd,YZbm), (Xat,Cd,YZbn),(Xat,Cd,YZbo),(Xat,Cd,YZbp),(Xat,Cd,YZbq),(Xat,Cd,YZbr),(Xat, Cd,YZbs),(Xat,Cd,YZbt),(Xat,Cd,YZbu),(Xat,Cd,YZbv),(Xat,Cd,YZbw),(Xat,Cd, YZbx),(Xat,Cd,YZby),(Xat,Cd,YZbz),(Xat,Cd,YZca),(Xat,Cd,YZcb),(Xat,Cd,YZcc),(Xat, Cd,YZcd),(Xat,Cd,YZce),(Xat,Cd,YZcf),(Xat,Cd,YZcg),(Xat,Cd,YZch),(Xat,Cd, YZci),(Xat,Cd,YZcj),(Xat,Cd,YZck),(Xat,Cd,YZcl),(Xat,Cd,YZcm),(Xat,Cd,YZcn),(Xat, Cd,YZco),(Xat,Cd,YZcp),(Xat,Cd,YZcq),(Xat,Cd,YZcr),(Xat,Cd,YZcs),(Xat,Cd, YZct),(Xat,Cd,YZcu),(Xat,Cd,YZcv),(Xat,Cd,YZcw),(Xat,Cd,YZcx),(Xat,Cd,YZcy),(Xat, Cd,YZcz),(Xat,Cd,YZda),(Xat,Cd,YZdb),(Xat,Cd,YZdc),(Xat,Cd,YZdd),(Xat,Cd, YZde),(Xat,Cd,YZdf),(Xat,Cd,YZdg),(Xat,Cd,YZdh),(Xat,Cd,YZdi),(Xat,Cd,YZdj),(Xat, Cd,YZdk),(Xat,Cd,YZdl),(Xat,Cd,YZdm),(Xat,Cd,YZdn),(Xat,Cd,YZdo),(Xat,Cd, YZdp),(Xat,Cd,YZdq),(Xat,Cd,YZdr),(Xat,Cd,YZds),(Xat,Cd,YZdt),(Xat,Cd,YZdu),(Xat, Cd,YZdv),(Xat,Cd,YZdw),(Xat,Cd,YZdx),(Xat,Cd,YZdy),(Xat,Cd,YZdz),(Xat,Cd, YZea),(Xat,Cd,YZeb),(Xat,Cd,YZec),(Xat,Cd,YZed),(Xat,Cd,YZee),(Xat,Cd,YZef),(Xat, Cd,YZeg),(Xat,Cd,YZeh),(Xat,Cd,YZei),(Xat,Cd,YZej),(Xat,Cd,YZek),(Xat,Cd,YZel), (Xat,Cd,YZem),(Xat,Cd,YZen),(Xat,Cd,YZeo),(Xat,Cd,YZep),(Xat,Cd,YZeq),(Xat, Cd,YZer),(Xat,Cd,YZes),(Xat,Cd,YZet),(Xat,Cd,YZeu),(Xat,Cd,YZev),(Xat,Cd,YZew), (Xat,Cd,YZex),(Xat,Cd,YZey),(Xat,Cd,YZez),(Xat,Cd,YZfa),(Xat,Cd,YZfb),(Xat, Cd,YZfc),(Xat,Cd,YZfd),(Xat,Cd,YZfe),(Xat,Cd,YZff),(Xat,Cd,YZfg),(Xat,Cd,YZfh), (Xat,Cd,YZfi),(Xat,Cd,YZfj),(Xat,Cd,YZfk),(Xat,Cd,YZfl),(Xat,Cd,YZfm),(Xat,Cd,YZfn), (Xat,Cd,YZfo),(Xat,Cd,YZfp),(Xat,Cd,YZfq),(Xat,Cd,YZfr),(Xat,Cd,YZfs),(Xat, Cd,YZft),(Xat,Cd,YZfu),(Xat,Cd,YZfv),(Xat,Cd,YZfw),(Xat,Cd,YZfx),(Xat,Cd,YZfy), (Xat,Cd,YZfz),(Xat,Cd,YZga),(Xat,Cd,YZgb),(Xat,Cd,YZgc),(Xat,Cd,YZgd),(Xat,Cd, YZge),(Xat,Cd,YZgf),(Xat,Cd,YZgg),(Xat,Cd,YZgh),(Xat,Cd,YZgi),(Xat,Cd,YZgj),(Xat, Cd,YZgk),(Xat,Cd,YZgl),(Xat,Cd,YZgm),(Xat,Cd,YZgn),(Xat,Cd,YZgo),(Xat,Cd, YZgp),(Xat,Cd,YZgq),(Xat,Cd,YZgr),(Xat,Cd,YZgs),(Xat,Cd,YZgt),(Xat,Ce,YZa),(Xat, Ce,YZb),(Xat,Ce,YZc),(Xat,Ce,YZd),(Xat,Ce,YZe),(Xat,Ce,YZf),(Xat,Ce,YZg),(Xat, Ce,YZh),(Xat,Ce,YZi),(Xat,Ce,YZj),(Xat,Ce,YZk),(Xat,Ce,YZl),(Xat,Ce,YZm),(Xat, Ce,YZn),(Xat,Ce,YZo),(Xat,Ce,YZp),(Xat,Ce,YZq),(Xat,Ce,YZr),(Xat,Ce,YZs),(Xat, Ce,YZt),(Xat,Ce,YZu),(Xat,Ce,YZv),(Xat,Ce,YZw),(Xat,Ce,YZx),(Xat,Ce,YZy),(Xat, Ce,YZz),(Xat,Ce,YZaa),(Xat,Ce,YZab),(Xat,Ce,YZac),(Xat,Ce,YZad),(Xat,Ce,YZae), (Xat,Ce,YZaf),(Xat,Ce,YZag),(Xat,Ce,YZah),(Xat,Ce,YZai),(Xat,Ce,YZaj),(Xat,Ce,YZak), (Xat,Ce,YZal),(Xat,Ce,YZam),(Xat,Ce,YZan),(Xat,Ce,YZao),(Xat,Ce,YZap),(Xat, Ce,YZaq),(Xat,Ce,YZar),(Xat,Ce,YZas),(Xat,Ce,YZat),(Xat,Ce,YZau),(Xat,Ce,YZav), (Xat,Ce,YZaw),(Xat,Ce,YZax),(Xat,Ce,YZay),(Xat,Ce,YZaz),(Xat,Ce,YZba),(Xat,Ce, YZbb),(Xat,Ce,YZbc),(Xat,Ce,YZbd),(Xat,Ce,YZbe),(Xat,Ce,YZbf),(Xat,Ce,YZbg), (Xat,Ce,YZbh),(Xat,Ce,YZbi),(Xat,Ce,YZbj),(Xat,Ce,YZbk),(Xat,Ce,YZbl),(Xat,Ce,YZbm), (Xat,Ce,YZbn),(Xat,Ce,YZbo),(Xat,Ce,YZbp),(Xat,Ce,YZbq),(Xat,Ce,YZbr),(Xat, Ce,YZbs),(Xat,Ce,YZbt),(Xat,Ce,YZbu),(Xat,Ce,YZbv),(Xat,Ce,YZbw),(Xat,Ce,YZbx), (Xat,Ce,YZby),(Xat,Ce,YZbz),(Xat,Ce,YZca),(Xat,Ce,YZeb),(Xat,Ce,YZcc),(Xat, Ce,YZcd),(Xat,Ce,YZce),(Xat,Ce,YZcf),(Xat,Ce,YZcg),(Xat,Ce,YZch),(Xat,Ce,YZci), (Xat,Ce,YZcj),(Xat,Ce,YZck),(Xat,Ce,YZcl),(Xat,Ce,YZcm),(Xat,Ce,YZcn),(Xat,Ce, YZco),(Xat,Ce,YZcp),(Xat,Ce,YZcq),(Xat,Ce,YZcr),(Xat,Ce,YZcs),(Xat,Ce,YZct),(Xat, Ce,YZcu),(Xat,Ce,YZcv),(Xat,Ce,YZcw),(Xat,Ce,YZcx),(Xat,Ce,YZcy),(Xat,Ce,YZcz), (Xat,Ce,YZda),(Xat,Ce,YZdb),(Xat,Ce,YZdc),(Xat,Ce,YZdd),(Xat,Ce,YZde),(Xat, Ce,YZdf),(Xat,Ce,YZdg),(Xat,Ce,YZdh),(Xat,Ce,YZdi),(Xat,Ce,YZdj),(Xat,Ce,YZdk), (Xat,Ce,YZdl),(Xat,Ce,YZdm),(Xat,Ce,YZdn),(Xat,Ce,YZdo),(Xat,Ce,YZdp),(Xat, Ce,YZdq),(Xat,Ce,YZdr),(Xat,Ce,YZds),(Xat,Ce,YZdt),(Xat,Ce,YZdu),(Xat,Ce,YZdv), (Xat,Ce,YZdw),(Xat,Ce,YZdx),(Xat,Ce,YZdy),(Xat,Ce,YZdz),(Xat,Ce,YZea),(Xat,Ce, YZeb),(Xat,Ce,YZec),(Xat,Ce,YZed),(Xat,Ce,YZee),(Xat,Ce,YZef),(Xat,Ce,YZeg),(Xat, Ce,YZeh),(Xat,Ce,YZei),(Xat,Ce,YZej),(Xat,Ce,YZek),(Xat,Ce,YZel),(Xat,Ce,YZem), (Xat,Ce,YZen),(Xat,Ce,YZeo),(Xat,Ce,YZep),(Xat,Ce,YZeq),(Xat,Ce,YZer),(Xat, Ce,YZes),(Xat,Ce,YZet),(Xat,Ce,YZeu),(Xat,Ce,YZev),(Xat,Ce,YZew),(Xat,Ce,YZex), (Xat,Ce,YZey),(Xat,Ce,YZez),(Xat,Ce,YZfa),(Xat,Ce,YZfb),(Xat,Ce,YZfc),(Xat,Ce, YZfd),(Xat,Ce,YZfe),(Xat,Ce,YZff),(Xat,Ce,YZfg),(Xat,Ce,YZfh),(Xat,Ce,YZfi),(Xat, Ce,YZfj),(Xat,Ce,YZfk),(Xat,Ce,YZfl),(Xat,Ce,YZfm),(Xat,Ce,YZfn),(Xat,Ce,YZfo), (Xat,Ce,YZfp),(Xat,Ce,YZfq),(Xat,Ce,YZfr),(Xat,Ce,YZfs),(Xat,Ce,YZft),(Xat,Ce,YZfu), (Xat,Ce,YZfv),(Xat,Ce,YZfw),(Xat,Ce,YZfx),(Xat,Ce,YZfy),(Xat,Ce,YZfz),(Xat,Ce, YZga),(Xat,Ce,YZgb),(Xat,Ce,YZgc),(Xat,Ce,YZgd),(Xat,Ce,YZge),(Xat,Ce,YZgf), (Xat,Ce,YZgg),(Xat,Ce,YZgh),(Xat,Ce,YZgi),(Xat,Ce,YZgj),(Xat,Ce,YZgk),(Xat,Ce, YZgl),(Xat,Ce,YZgm),(Xat,Ce,YZgn),(Xat,Ce,YZgo),(Xat,Ce,YZgp),(Xat,Ce,YZgq),(Xat, Ce,YZgr),(Xat,Ce,YZgs),(Xat,Ce,YZgt),(Xat,Cf,YZa),(Xat,Cf,YZb),(Xat,Cf,YZc), (Xat,Cf,YZd),(Xat,Cf,YZe),(Xat,Cf,YZf),(Xat,Cf,YZg),(Xat,Cf,YZh),(Xat,Cf,YZi),(Xat, Cf,YZj),(Xat,Cf,YZk),(Xat,Cf,YZl),(Xat,Cf,YZm),(Xat,Cf,YZn),(Xat,Cf,YZo),(Xat, Cf,YZp),(Xat,Cf,YZq),(Xat,Cf,YZr),(Xat,Cf,YZs),(Xat,Cf,YZt),(Xat,Cf,YZu),(Xat, Cf,YZv),(Xat,Cf,YZw),(Xat,Cf,YZx),(Xat,Cf,YZy),(Xat,Cf,YZz),(Xat,Cf,YZaa),(Xat, Cf,YZab),(Xat,Cf,YZac),(Xat,Cf,YZad),(Xat,Cf,YZae),(Xat,Cf,YZaf),(Xat,Cf,YZag),(Xat, Cf,YZah),(Xat,Cf,YZai),(Xat,Cf,YZaj),(Xat,Cf,YZak),(Xat,Cf,YZal),(Xat,Cf,YZam), (Xat,Cf,YZan),(Xat,Cf,YZao),(Xat,Cf,YZap),(Xat,Cf,YZaq),(Xat,Cf,YZar),(Xat,Cf, YZas),(Xat,Cf,YZat),(Xat,Cf,YZau),(Xat,Cf,YZav),(Xat,Cf,YZaw),(Xat,Cf,YZax),(Xat, Cf,YZay),(Xat,Cf,YZaz),(Xat,Cf,YZba),(Xat,Cf,YZbb),(Xat,Cf,YZbc),(Xat,Cf,YZbd), (Xat,Cf,YZbe),(Xat,Cf,YZbf),(Xat,Cf,YZbg),(Xat,Cf,YZbh),(Xat,Cf,YZbi),(Xat,Cf, YZbj),(Xat,Cf,YZbk),(Xat,Cf,YZbl),(Xat,Cf,YZbm),(Xat,Cf,YZbn),(Xat,Cf,YZbo),(Xat, Cf,YZbp),(Xat,Cf,YZbq),(Xat,Cf,YZbr),(Xat,Cf,YZbs),(Xat,Cf,YZbt),(Xat,Cf,YZbu), (Xat,Cf,YZbv),(Xat,Cf,YZbw),(Xat,Cf,YZbx),(Xat,Cf,YZby),(Xat,Cf,YZbz),(Xat,Cf, YZca),(Xat,Cf,YZcb),(Xat,Cf,YZcc),(Xat,Cf,YZcd),(Xat,Cf,YZce),(Xat,Cf,YZcf),(Xat, Cf,YZcg),(Xat,Cf,YZch),(Xat,Cf,YZci),(Xat,Cf,YZcj),(Xat,Cf,YZck),(Xat,Cf,YZcl), (Xat,Cf,YZcm),(Xat,Cf,YZcn),(Xat,Cf,YZco),(Xat,Cf,YZcp),(Xat,Cf,YZcq),(Xat,Cf, YZcr),(Xat,Cf,YZcs),(Xat,Cf,YZct),(Xat,Cf,YZcu),(Xat,Cf,YZcv),(Xat,Cf,YZcw),(Xat, Cf,YZcx),(Xat,Cf,YZcy),(Xat,Cf,YZcz),(Xat,Cf,YZda),(Xat,Cf,YZdb),(Xat,Cf,YZdc), (Xat,Cf,YZdd),(Xat,Cf,YZde),(Xat,Cf,YZdf),(Xat,Cf,YZdg),(Xat,Cf,YZdh),(Xat,Cf, YZdi),(Xat,Cf,YZdj),(Xat,Cf,YZdk),(Xat,Cf,YZdl),(Xat,Cf,YZdm),(Xat,Cf,YZdn),(Xat, Cf,YZdo),(Xat,Cf,YZdp),(Xat,Cf,YZdq),(Xat,Cf,YZdr),(Xat,Cf,YZds),(Xat,Cf,YZdt), (Xat,Cf,YZdu),(Xat,Cf,YZdv),(Xat,Cf,YZdw),(Xat,Cf,YZdx),(Xat,Cf,YZdy),(Xat,Cf, YZdz),(Xat,Cf,YZea),(Xat,Cf,YZeb),(Xat,Cf,YZec),(Xat,Cf,YZed),(Xat,Cf,YZee),(Xat, Cf,YZef),(Xat,Cf,YZeg),(Xat,Cf,YZeh),(Xat,Cf,YZei),(Xat,Cf,YZej),(Xat,Cf,YZek), (Xat,Cf,YZel),(Xat,Cf,YZem),(Xat,Cf,YZen),(Xat,Cf,YZeo),(Xat,Cf,YZep),(Xat,Cf,YZeq), (Xat,Cf,YZer),(Xat,Cf,YZes),(Xat,Cf,YZet),(Xat,Cf,YZeu),(Xat,Cf,YZev),(Xat, Cf,YZew),(Xat,Cf,YZex),(Xat,Cf,YZey),(Xat,Cf,YZez),(Xat,Cf,YZfa),(Xat,Cf,YZfb), (Xat,Cf,YZfc),(Xat,Cf,YZfd),(Xat,Cf,YZfe),(Xat,Cf,YZff),(Xat,Cf,YZfg),(Xat,Cf,YZfh), (Xat,Cf,YZfi),(Xat,Cf,YZfj),(Xat,Cf,YZfk),(Xat,Cf,YZfl),(Xat,Cf,YZfm),(Xat,Cf,YZfn), (Xat,Cf,YZfo),(Xat,Cf,YZfp),(Xat,Cf,YZfq),(Xat,Cf,YZfr),(Xat,Cf,YZfs),(Xat,Cf, YZft),(Xat,Cf,YZfu),(Xat,Cf,YZfv),(Xat,Cf,YZfw),(Xat,Cf,YZfx),(Xat,Cf,YZfy),(Xat, Cf,YZfz),(Xat,Cf,YZga),(Xat,Cf,YZgb),(Xat,Cf,YZgc),(Xat,Cf,YZgd),(Xat,Cf,YZge), (Xat,Cf,YZgf),(Xat,Cf,YZgg),(Xat,Cf,YZgh),(Xat,Cf,YZgi),(Xat,Cf,YZgj),(Xat,Cf,YZgk), (Xat,Cf,YZgl),(Xat,Cf,YZgm),(Xat,Cf,YZgn),(Xat,Cf,YZgo),(Xat,Cf,YZgp),(Xat, Cf,YZgq),(Xat,Cf,YZgr),(Xat,Cf,YZgs),(Xat,Cf,YZgt).

The invention disclosed herein is also meant to encompass prodrugs of the disclosed compounds. Prodrugs are considered to be any covalently bonded carriers that release the active parent drug in vivo. Non-limiting examples of prodrugs include esters or amides of compounds of Formulae I, I′, I″, II or II′ having hydroxyalkyl or aminoalkyl as a substituent, and these can be prepared by reacting such compounds with anhydrides such as succinic anhydride.

The invention disclosed herein is also meant to encompass the disclosed compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹¹C, ¹³c, ¹⁴c, ¹⁵N, ¹⁸O, ¹⁷O, ³¹P, ³²P, ³⁵S, ¹⁸F, and ³⁶Cl, respectively, and preferably ³H, ¹¹C, and ¹⁴C. Isotopically-labeled compounds of the present invention can be prepared by methods known in the art.

The present invention is also directed specifically to ³H, ¹¹C, and ¹⁴ C radiolabeled compounds of Formula I, I′, I″, II or II′ as well as their pharmaceutically acceptable salts, prodrugs and solvates, and the use of any such compounds as radioligands for their binding site on the calcium channel. For example, one use of the labeled compounds of the present invention is the characterization of specific receptor binding. Another use of the labeled compounds of the present invention is an alternative to animal testing for the evaluation of structure-activity relationships. For example, the receptor assay may be performed at a fixed concentration of a labeled compound of Formula I, I′, I″, II or II′ and at increasing concentrations of a test compound in a competition assay. For example, tritiated compounds of any of Formula I, I′, I″, II or II′ can be prepared by introducing tritium into the particular compound of Formula I, I′, I″, II or II′, for example, by catalytic dehalogenation with tritium. This method may include reacting a suitably halogen-substituted precursor of a compound of Formula I, I′, I″, II or II′ with tritium gas in the presence of a suitable catalyst, for example, Pd/C, in the presence or absence of a base. Other suitable methods for preparing tritiated compounds can be found in Filer, Isotopes in the Physical and Biomedical Sciences, Vol. 1, Labeled Compounds (Part A), Chapter 6 (1987). ¹⁴C-labeled compounds can be prepared by employing starting materials having a ¹⁴C carbon.

Some of the compounds disclosed herein may contain one or more asymmetric centers and may thus give rise to enantiomers, diastereomers, and other stereoisomeric forms. The present invention is meant to encompass the uses of all such possible forms, as well as their racemic and resolved forms and mixtures thereof. The individual enantiomers may be separated according to methods known to those of ordinary skill in the art in view of the present disclosure. When the compounds described herein contain olefinic double bonds or other centers of geometric asymmetry, and unless specified otherwise, it is intended that they include both E and Z geometric isomers. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction

The term “racemic” refers to a mixture of equal parts of enantiomers and which mixture is optically inactive.

The term “resolution” refers to the separation or concentration or depletion of one of the two enantiomeric forms of a molecule.

The terms “a” and “an” refer to one or more.

The invention disclosed herein also encompasses the use of all salts of the disclosed compounds, including all non-toxic pharmaceutically acceptable salts thereof of the disclosed compounds. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, hydrofluoride, phosphate, sulfate, nitrate and the like; organic acid salts such as citrate, lactate, tartrate, maleate, fumarate, mandelate, acetate, dichloroacetate, trifluoroacetate, oxalate, formate, succinate, and the like; sulfonates such as methanesulfonate, benzenesulfonate, p-toluenesulfonate and the like; and amino acid salts such as arginate, asparginate, glutamate and the like.

Acid addition salts can be formed by mixing a solution of the particular compound of the present invention with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, and the like. Basic salts can be formed by mixing a solution of the particular compound of the present invention with a solution of a pharmaceutically acceptable non-toxic base such as sodium hydroxide, potassium hydroxide, choline hydroxide, sodium carbonate and the like.

The invention disclosed herein is also meant to encompass solvates of the disclosed compounds. One type of solvate is a hydrate. Solvates typically do not contribute significantly to the physiological activity or toxicity of the compounds and as such can function as pharmacological equivalents.

Some compounds of the present invention may have one or more of the following characteristics:

high affinity of calcium (Ca²⁺) channels, especially N-type calcium channels,

high selectivity to calcium (Ca²⁺) channels, especially N-type calcium channels than other channels,

reduced side effect,

high stability

high oral absorbability,

high bioavailability,

low clearance,

easily transfers to brain

long half-life,

long efficacy of a medicine and /or

high protein-unbound fraction.

These compounds are considered useful as blockers of calcium (Ca²⁺) channels, especially N-type calcium channels.

Since compounds of Formula I or I′ are blockers of calcium (Ca²⁺) channels, a number of diseases and conditions mediated by calcium ion influx can be treated by employing these compounds. Therefore, the present invention provides a method of treating, preventing or ameliorating stroke, neuronal damage resulting from head trauma, epilepsy, pain (e.g., acute pain, chronic pain, which includes but is not limited to, neuropathic pain and inflammatory pain, or surgical pain), migraine, a mood disorder, schizophrenia, a neurodegenerative disorder (e.g., Alzheimer's disease, amyotrophic lateral sclerosis (ALS), or Parkinson's disease), depression, anxiety, a psychosis, hypertension, or cardiac arrhythmia. In one embodiment, the invention provides a method of treating pain. In another embodiment, the type of pain treated is chronic pain. In another embodiment, the type of pain treated is neuropathic pain. In another embodiment, the type of pain treated is inflammatory pain. In another embodiment, the type of pain treated is acute pain. In each instance, such method of treatment, prevention, or amelioration require administering to an animal in need of such treatment, prevention or amelioration an amount of a compound of the present invention that is therapeutically effective in achieving said treatment, prevention or amelioration. In one embodiment, the amount of such compound is the amount that is effective as to block calcium channels in vivo.

Chronic pain includes, but is not limited to, neuropathic pan, inflammatory pain, postoperative pain, cancer pain, osteoarthritis pain associated with metastatic cancer, trigeminal neuralgia, acute herpetic and postherpetic neuralgia, diabetic neuropathy, causalgia, brachial plexus avulsion, occipital neuralgia, reflex sympathetic dystrophy, fibromyalgia, gout, phantom limb pain, bum pain, and other forms of neuralgia, neuropathic, and idiopathic pain syndromes.

Chronic somatic pain generally results from inflammatory responses to tissue injury such as nerve entrapment, surgical procedures, cancer or arthritis (Brower, Nature Biotechnology 2000; 18: 387-391).

The inflammatory process is a complex series of biochemical and cellular events activated in response to tissue injury or the presence of foreign substances (Levine, Inflammatory Pain, In: Textbook of Pain, Wall and Melzack eds., 3^{rd} ed., 1994). Inflammation often occurs at the site of injured tissue, or foreign material, and contributes to the process of tissue repair and healing. The cardinal signs of inflammation include erythema (redness), heat, edema (swelling), pain and loss of function (ibid.). The majority of patients with inflammatory pain do not experience pain continually, but rather experience enhanced pain when the inflamed site is moved or touched. Inflammatory pain includes, but is not limited to, osteoarthritis and rheumatoid arthritis.

Chronic neuropathic pain is a heterogenous disease state with an unclear etiology. In chronic neuropathic pain, the pain can be mediated by multiple mechanisms. This type of pain generally arises from injury to the peripheral or central nervous tissue. The syndromes include pain associated with spinal cord injury, multiple sclerosis, post-herpetic neuralgia, trigeminal neuralgia, phantom pain, causalgia, and reflex sympathetic dystrophy and lower back pain. The chronic pain is different from acute pain in that patients suffer the abnormal pain sensations that can be described as spontaneous pain, continuous superficial burning and/or deep aching pain. The pain can be evoked by heat-, cold-, and mechano-hyperalgesia or by heat-, cold-, or mechano-allodynia.

Neuropathic pain can be caused by injury or infection of peripheral sensory nerves. It includes, but is not limited to, pain from peripheral nerve trauma, herpes virus infection, diabetes mellitus, causalgia, plexus avulsion, neuroma, limb amputation, and vasculitis. Neuropathic pain is also caused by nerve damage from chronic alcoholism, human immunodeficiency virus infection, hypothyroidism, uremia, or vitamin deficiencies. Stroke (spinal or brain) and spinal cord injury can also induce neuropathic pain. Cancer-related neuropathic pain results from tumor growth compression of adjacent nerves, brain, or spinal cord. In addition, cancer treatments, including chemotherapy and radiation therapy, can also cause nerve injury. Neuropathic pain includes but is not limited to pain caused by nerve injury such as, for example, the pain from which diabetics suffer.

The present invention is also directed more generally to a method for treating a disorder responsive to the blockade of calcium channels, and particularly the selective blockade of N-type calcium channels, in an animal suffering from said disorder, said method comprising administering to the animal an effective amount of a compound represented by any of defined Formula I, I′, I″, II or II′, or a pharmaceutically acceptable salt, prodrug or solvate thereof.

The present invention is also directed to the use of a compound represented by any of defined Formula I, I′, I″, II or II′, or a pharmaceutically acceptable salt, prodrug or solvate thereof, in the manufacture of a medicament for treating a disorder responsive to the blockade of calcium channels in an animal suffering from said disorder. In one embodiment, the disorder is responsive to the selective blockade of N-type calcium channels.

Furthermore, the present invention is directed to a method of modulating calcium channels, especially N-type calcium channels, in an animal in need thereof, said method comprising administering to the animal at least one compound represented by any of defined Formula I, I′, I″, II or II′, or a pharmaceutically acceptable salt, prodrug or solvate thereof.

The present invention is also directed to the use of a compound represented by any of defined Formula I or I′, or a pharmaceutically acceptable salt, prodrug or solvate thereof, in the manufacture of a medicament for modulating calcium channels, especially N-type calcium channels, in an animal in need thereof.

Synthesis of Compounds

The compounds of the present invention can be prepared in a number of ways well known to those skilled in the art. The compounds of the present invention can be synthesized using the methods outlined below, together with methods known in the art of synthetic organic chemistry, or variations thereof as appreciated by those skilled in the art. Preferred methods include, but are not limited to, those described below.

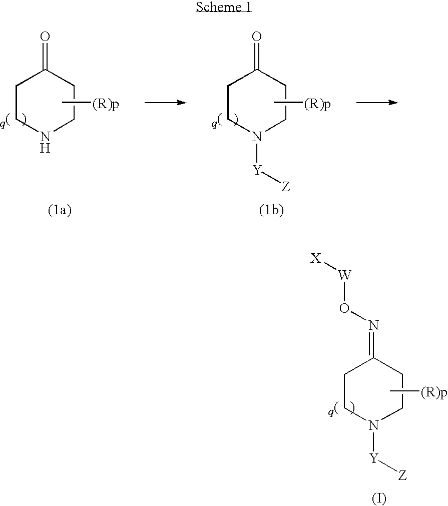

In order to generate compounds of general formula (I) a multi-step reaction sequence as described in Scheme I can be employed. Herein, a piperidone or the corresponding equivalent (1a), wherein R and p are as defined above, is reacted with an acid (Z-Y—OH, Y: C═O), wherein Z is as defined above. Typically the reaction is effected using standard amide coupling conditions, familiar to one skilled in the art, such as N-ethyldimethylaminopropylcarbodiimide hydrochloride/1 -Hydroxybenzotriazole. The acid chloride (Z-Y—Cl, Y: C═O), acid anhydride (Z-Y-Z, Y: C═O) or sulfonyl chloride (Z-Y—Cl, Y: S═O) can also be coupled to amine (1a) by using standard conditions, familiar to one skilled in the art. The resultant ketone (1b) can then be coupled with an O-substituted hydroxylamine (X—W—O—NH₂) or the corresponding salt, e.g. hydrochloric acid salt or trifluoroacetic acid salt, wherein X and W are as defined above, in a suitable solvent such as ethanol, in the presence of a base such as sodium acetate to generate the desired oxime (I). The O-substituted hydroxylamines (X—W—O—NH₂) are either commercially available or can be readily prepared using procedures familiar to one skilled in the art. Non-limiting examples of such preparations have been described by e.g. J. N. Kim et al.: Synthetic Communications, (1992) 22, 1427-1432, H. M. Petrassi et al.: Organic Letters (2001), 3, 139-142, E. Grochowski, J. Jurczak: Synthesis (1976), 682-684, WO02/06213 and WO2005/054179. Typical but non-limiting synthetic routes to obtain O-substituted hydroxylamines of general formula (2c) is illustrated in Scheme 2.

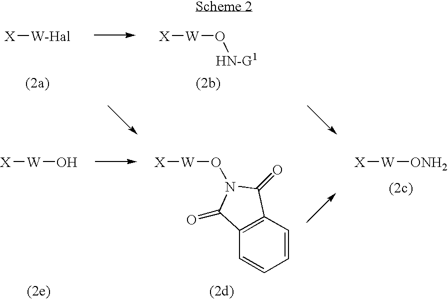

Reaction of N-hydroxyphthalimide or N-protected hydroxylamines (G¹-NH—OH; G¹ is an amino protecting group such as N-tert-butoxycarbonyl, N-benzyloxycarbonyl or N-phthalimide) with a halide or the corresponding equivalent (2a, Hal=Cl, Br, I, OTs etc.) wherein X and W are as defined above, in a suitable solvent in the presence of a base such as triethylamine, 1,8-diazabicyclo[5,4,0]-undec-7-ene (DBU), K₂CO₃ afford intermediates (2b) or (2d) respectively. Alternatively an alcohol (2e) wherein X and W are as defined above, can be reacted with N-hydroxyphthalimide in a Mitsunobu-like reaction in the presence of triphenylphosphine and diethyl azodicarboxylate (DEAD) or diisopropyl azodicarboxyrate in a suitable solvent such as tetrahydrofuran, affording compounds (2d). Reaction of (2d) with hydrazine monohydrate or methylhydrazine affords the desired O-substituted hydroxylamines (2c). Treatment of intermediates (2b) with acid, e.g. trifluoroacetic acid or hydrochloric acid also yields the desired O-substituted hydroxylamines (2c). The O-substituted hydroxylamines (2c) can be isolated and used either as free amines or as the corresponding salts, e.g. hydrochloric acid salts or trifluoroacetic acid salts.

Alternative ways of preparing some of the compounds of the present invention are detailed in Schemes 3 to 10.

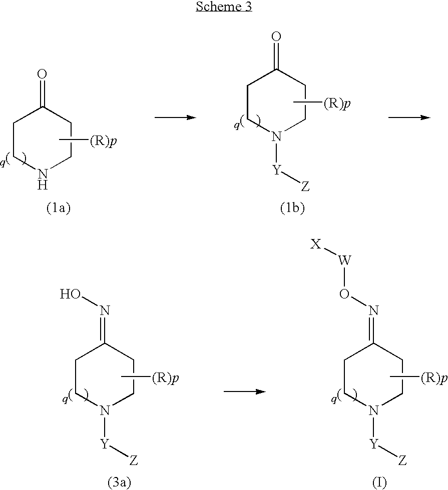

wherein R, p, q, X, Y, Z and W are as defined above.

As an alternative to Scheme 1, Scheme 3 employs a hydroxylimine (3a) which can be prepared by similar conditions as described for the generation of oximes (I) in Scheme 1. Alkylation of a hydroxylimine (3a) with a halide or the corresponding equivalent (X—W-Hal; Hal=Cl, Br, I, OTs etc.), in a suitable solvent such as N,N-dimethylformamide, in the presence of a base such as NaH can generate the desired oxime (I).

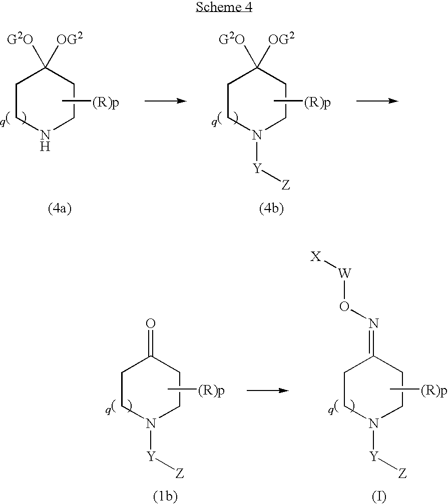

wherein R, p, q, X, Y, Z and W are as defined above, and G² is a hydroxy protecting group such as N-tert-butoxycarbonyl, N-benzyloxycarbonyl or N-benzyl.

As an alternative to Scheme 1 and 3, Scheme 4 employs a suitably protected piperidone (4a), whereby G²O—C-OG² is, for example, 1,3-dioxolane. This can be subjected to alkylation with a halide or the corresponding equivalent (Z-Y-Hal; Hal=Cl, Br, I, OTs etc.), in a suitable solvent such as acetonitrile, in the presence of a base such as K₂CO₃ to yield a suitably protected alkylated piperidone (4b).

Deprotection can be accomplished using standard conditions, familiar to one skilled in the art. The O-substituted hydroxylamine (X—W—O—NH₂) or the corresponding hydrochloride can then be coupled to the free ketone (1b) in an analogous manner to previously described in Scheme 1.

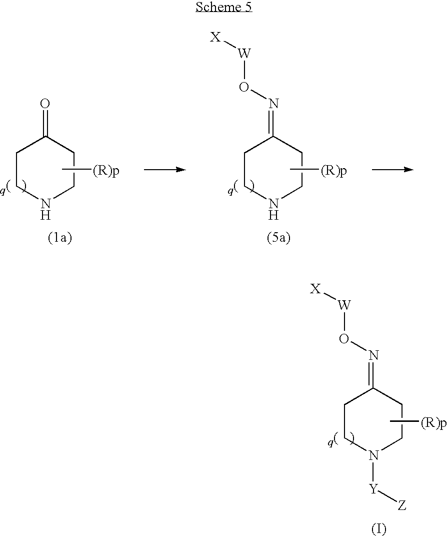

wherein R, p, q, X, Y, Z and W are as defined above.

As an alternative to Scheme 1, 3 and 4, in Scheme 5, a piperidone or the corresponding equivalent (1a), wherein R and p are as defined above, is first reacted with an O-substituted hydroxylamine (X—W—O—NH₂) or the corresponding hydrochloride by similar conditions as described for the generation of oximes (I) in Scheme 1. The resultant oxime (5a) can then be coupled with an acid (Z-Y—OH, Y: C═O), acid chloride (Z-Y—Cl, Y: C═O), acid anhydride (Z-Y-Z, Y: C═O) or sulfonyl chloride (Z-Y—Cl, Y: S═O) by similar conditions as described for the generation of the ketone (1b) in Scheme 1 to generate the desired oxime (I). A halide or the corresponding equivalent (Z-Y-Hal; Hal=Cl, Br, I, OTs etc.) can also be reacted with the oxime (5a) by similar conditions as described for the generation of the alkylated piperidone (4b) in Scheme 4. The isocyanate, thioisocyanate or the corresponding equivalent can also be coupled to the oxime (5a) by using standard conditions, familiar to one skilled in the art.

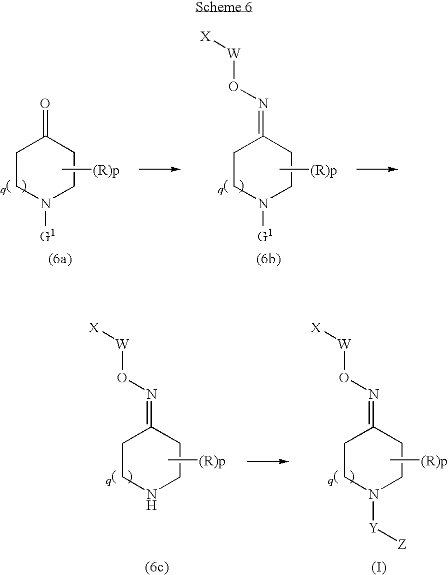

wherein R, p, q, X, Z, W and G¹ are as defined above, Y═CO or SO₂.

As an alternative to Scheme 1 and 3 to 5, Scheme 6 employs a suitably protected piperidone (6a), whereby G¹ is, for example, t-butyloxycarbonyl. This can be coupled with an O-substituted hydroxylamine (X—W—O—NH₂) or the corresponding hydrochloride by similar conditions as described for the generation of oximes (I) in Scheme 1. Deprotection can be accomplished using standard conditions, familiar to one skilled in the art. The acid (Z-Y—OH, Y: C═O), acid chloride (Z-Y—Cl, Y: C═O), acid anhydride (Z-Y-Z, Y: C═O) or sulfonyl chloride (Z-Y—Cl, Y: SO₂), can be coupled with free amine (6c) in an analogous manner to previously described in Scheme 1. The isocyanate or thioisocyanate can also be coupled to amine (6c) by using standard conditions, familiar to one skilled in the art.

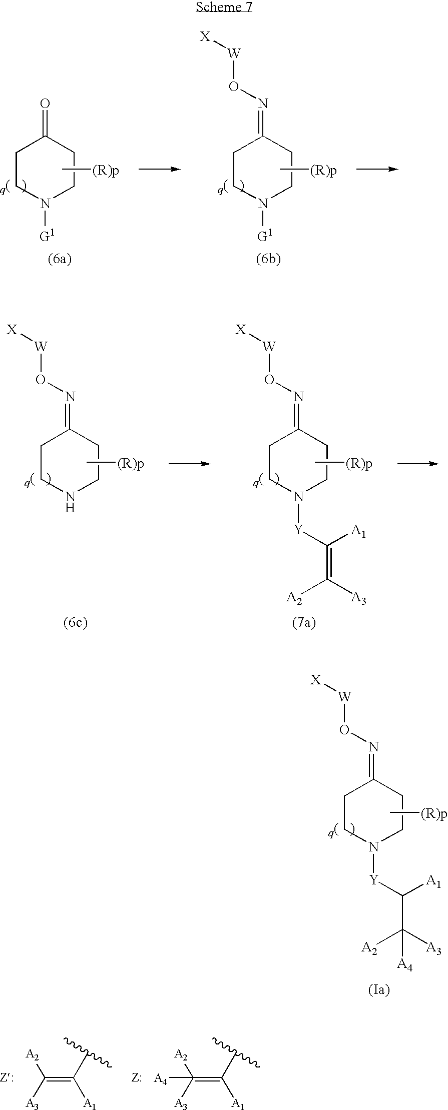

wherein R, p, q, X, W and G¹ are as defined above, Y is CO or SO₂, and A₁, A₂, A₃ and A₄ are each independently a substituent for “optionally substituted lower alkyl” or “optionally substituted lower alkenyl.”

Intermediate amine (6c) can be further utilized to synthesize compounds of general formula (Ia). An optionally substituted acrylic acid (Z′-Y—OH, Y: C═O), acryl chloride (Z′-Y—Cl, Y: C═O), acrylic anhydride (Z′-Y-Z′, Y: C═O), vinylsulfonyl chloride (Z′-Y—Cl, Y: SO₂) or the corresponding equivalent can be coupled with free amine (6c) in an analogous manner to previously described in Scheme 1. The resulting compound (7a) can react with a nucleophile (A₄-H) such as an amine or an alcohol to give the desired oxime (Ia).

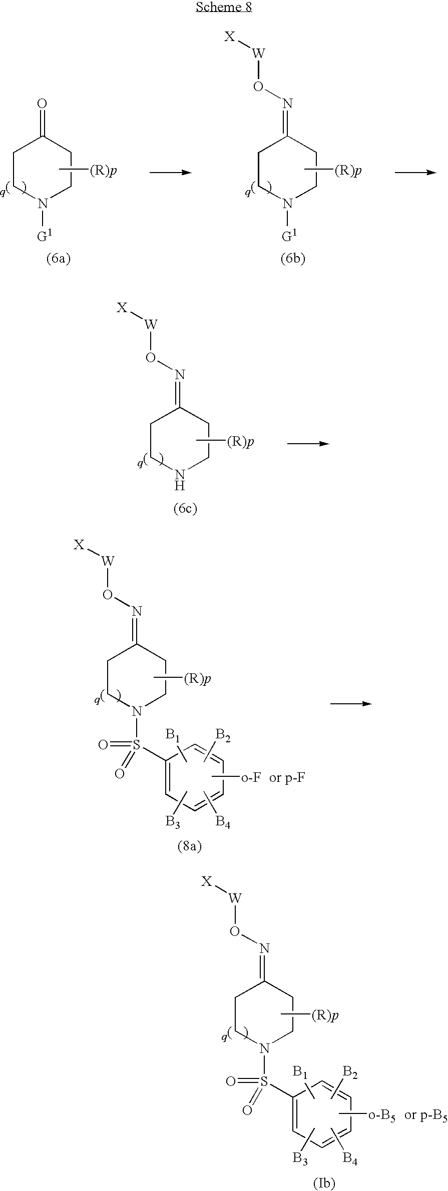

wherein R, p, q, X, W and G¹ are as defined above, and B₁, B₂, B₃, B₄ and B₅ are each independently a substituent for “optionally substituted aryl.”

Intermediate amine (6c) can be further utilized to synthesize compounds of general formula (1b). An optionally substituted acrylic acid (Z′-Y—OH, Y: C═O), acryl chloride (Z′-Y—Cl, Y: C═O), acrylic anhydride (Z′-Y-Z′, Y: C═O), vinylsulfonyl chloride (Z′-Y—Cl, Y: SO₂) or the corresponding equivalent can be coupled with free amine (6c) in an analogous manner to previously described in Scheme 1. The resulting compound (8a) can react with a nucleophile (B₅—H) such as an amine or an alcohol to give the desired oxime (1b).

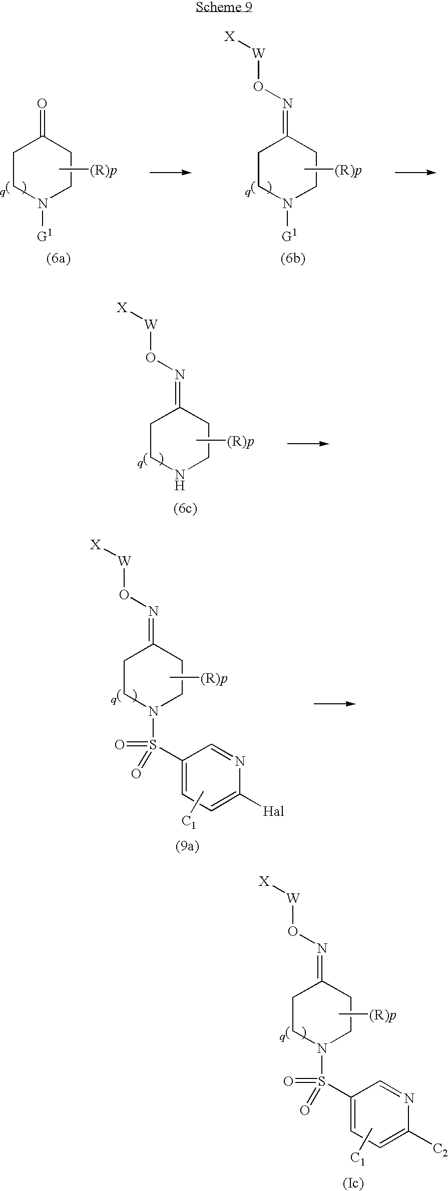

wherein R, p, q, X, W and G¹ are as defined above, C₁ and C₂ are each independently a substituent for “optionally substituted heteroaryl” and Hal are halogen. Intermediate amine (6c) can also be further utilized to synthesize compounds of general formula (Ic). Optionally substituted halopyridinesulfonyl chloride can be coupled with free amine (6c) in an analogous manner to previously described in Scheme 1. The resulting compound (9a) can react with a nucleophile (C₂—H) such as an amine or an alcohol to give the desired oxime (Ic).

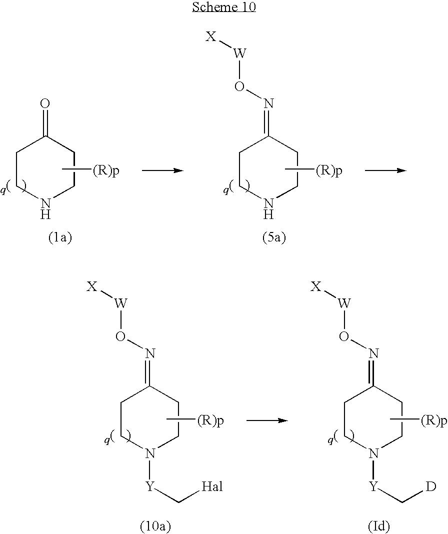

wherein R, p, q, X, Y and W are as defined above, Y is CO or SO₂, and Hal is halogen. Intermediate amine (5a) can be further utilized to synthesize compounds of general formula (Id). A halogen substituted acid (Hal-CH₂—Y—OH, Y: C═O), acid chloride (Hal-CH₂—Y—Cl, Y: C═O), acid anhydride (Hal-CH₂—Y—CH₂-Hal, Y: C═O), sulfonyl chloride (Hal-CH₂—Y—Cl, Y: S═O) or the corresponding equivalent can be coupled with free amine (5a) in an analogous manner to previously described in Scheme 1. The resulting compound (10a) can react with a nucleophile (D-H) such as an amine or an alcohol to give the desired oxime (Id).

Testing of Compounds

Representative compounds of the present invention were assessed by calcium mobilization and/or electrophysiological assays for calcium channel blocker activity. One aspect of the present invention is based on the use of the compounds herein described as N-type calcium channel blockers. In one aspect of the present invention, it has been found that certain compounds herein described show selectivity as N-type calcium channel blockers. Based upon this property, these compounds are considered useful in treating, preventing, or ameliorating stroke, neuronal damage resulting from head trauma, migraine, epilepsy, a mood disorder, schizophrenia, a neurodegenerative disorder (such as, e.g., Alzheimer's disease, ALS, or Parkinson's disease), a psychosis, depression, anxiety, hypertension, or cardiac arrhythmia. The compounds of the present invention are also expected to be effective in treating, preventing or ameliorating pain, such as acute pain, chronic pain, which includes but is not limited to, neuropathic pain and inflammatory pain or surgical pain.

More specifically, the present invention is directed to compounds of Formula I, I′, I″, II or II′ that are blockers of calcium channels. According to the present invention, those compounds having preferred N-type calcium channel blocking properties exhibit an IC₅₀ of about 100 μM or less in the calcium mobilization and/or electrophysiological assays described herein. Preferably, the compounds of the present invention exhibit an IC₅₀ of 10 μM or less. Most preferably, the compounds of the present invention exhibit an IC₅₀ of about 1.0 μM or less. Compounds of the present invention can be tested for their N-type and L-type Ca²⁺ channel blocking activity by the following calcium mobilization and/or electrophysiological assays.

In one embodiment, compounds useful in the present invention are those represented by any one of Formula I, I′, I″, II or II′ that exhibit selectivity for N-type calcium channels over L-type calcium channels in the calcium mobilization and/or electrophysiological assays described herein. The phrase “selectivity for N-type calcium channels over L-type calcium channels” is used herein to mean that the ratio of an IC₅₀ for L-type channel blocking activity for a compound of the present invention over an IC₅₀ for N-type channel blocking activity for the same compound is more than 1, i.e., LTCC IC₅₀/NTCC IC₅₀>1. Preferably, compounds of the present invention exhibit an LTCC IC₅₀/NTCC IC₅₀ ratio of about 2 or more, about 10 or more, about 20 or more, about 30 or more, about 50 or more, or about 100 or more.

Calcium Mobilization and Electrophysiological Assay Protocols:

Cell maintenance and differentiation. Unless noted otherwise, cell culture reagents were purchased from Mediatech of Hemdon, Md. IMR32 cells (American Type Culture Collection, ATCC, Manassas, Va.) were routinely cultured in growth medium consisting of minimum essential medium containing 10% fetal bovine serum (FBS, Hyclone, Logan, Utah), 100 U/mL penicillin, 100 μg/mL streptomycin, 2 mM L-glutamine, 1 mM sodium pyruvate, and 1× MEM non-essential amino acids. 80-90% confluent flasks of cells were differentiated using the following differentiation medium: Growth medium plus 1 mM dibutyryl cyclic AMP (Sigma, St. Louis, Mo.), and 2.5 μM bromodeoxyuridine (Sigma). Cells were differentiated for 8 days by replacing differentiation medium every 2-3 days.

A7r5 (ATCC) cells were maintained and routinely cultured in A7r5 growth medium consisting of Dulbecco's Modified Eagles Medium containing 10% FBS, 100 U/mL penicillin, 100 μg/mL streptomycin, 4 mM L-glutamine, and 0.15% sodium bicarbonate. 80-90% confluent flasks of cells were differentiated using the following differentiation medium: A7r5 Growth Medium plus 1 mM dibutyryl cyclic AMP (Sigma). Cells were differentiated for 8 days by replacing differentiation medium every 2-3 days.

Recombinant human embryonal kidney cells (HEK293, ATCC) stably transfected with either N-type calcium channel (NTCC) subunits (α1b, α2δ, and β3) or L-type calcium channel (LTCC) subunits (α1c, α2δ, and β1) were routinely cultured in growth medium consisting of Dulbecco's Modified Eagles Medium containing 10% FBS, 100 U/mL penicillin, 100 μg/mL streptomycin, 4 mM L-glutamine, 500 μg/mL geneticin (G418), 20 μg/mL Blasticidin S (InVivogen, San Diego, Calif.) and 500 μg/mL zeocin (InVivogen).

FLIPR Calcium Mobilization Assay for N-type Calcium Channel. One day prior to performing this assay, differentiated IMR32 cells were treated with 1× CellStripper, and seeded on poly-D-lysine-coated 96-well clear-bottom black plates (Becton Dickinson, Franklin Lakes, N.J.) at 200,000 cells/well. On the day of the assay, the cell plates were washed with IMR32 buffer (127 mM NaCl, 1 mM KCl, 2 mM MgCl₂, 700 μM NaH₂PO₄, 5 mM CaCl₂, 5 mM NaHCO₃, 8 mM HEPES, 10 mM glucose, pH 7.4), then pre-stimulated with KCl and loaded as follows: 0.05 mL of IMR32 buffer, 0.05 mL of each compound tested diluted in IMR32 buffer containing 20 μM nitrendipine (Sigma), and 0.1 mL KCl dissolved in IMR32 buffer, plus Fluo-4 were added (3 μM final concentration, Molecular Probes, Eugene, Oreg.). Final test compound concentrations ranged from about 846 pM to about 17 μM, final nitrendipine concentration was 5 μM, and final KCl concentration was 90 mM. After 1 hour, the cells were washed twice with 0.05 mL of each compound tested in nitrendipine-containing IMR32 buffer (no KCl or Fluo-4), and then replaced with 0.1 mL of each compound tested in nitrendipine-containing IMR32 buffer. Plates were then transferred to a Fluorimetric imaging Plate Reader (FLIPR⁹⁶, Molecular Devices, Inc., Sunnyvale, Calif.) for assay. The FLIPR measured basal Fluo-4 fluorescence for 315 seconds (i.e., 5 minutes and 15 seconds), then added 0.1 mL KCl agonist dissolved in IMR32 buffer and measured fluorescence for another 45 seconds. Final test compound concentrations on the cells after FLIPR read ranged from about 846 pM to about 17 μM, final nitrendipine concentration was 5 μM, and final KCl concentration was 90 mM. Data were collected over the entire time course and analyzed using Excel, Graph Pad Prism (version 3.02, Graph Pad, San Diego, Calif.), or an in-house non-linear regression analysis software.

FLIPR Calcium Mobilization Assay for L-type Calcium Channel. One day prior to performing this assay, HEK293 cells stably expressing recombinant rat L-type calcium channel (LTCC) subunits (α1c, α2δ, and β1) were trypsinized, then seeded on poly-D-lysine-coated 96-well clear-bottom black plates (Becton Dickinson, Franklin Lakes, N.J.) at 75,000 cells/well. On the day of the assay, the plates were washed with LTCC wash buffer (127 mM NaCl, 2 mM MgCl₂, 700 μM NaH₂PO₄, 5 nM CaCl₂, 5 mM NaHCO₃, 8 mM HEPES, 10 mM glucose, pH 7.4), then loaded with 0.1 mL of LTCC wash buffer containing Fluo-4 (3 μM final concentration, Molecular Probes, Eugene, Oreg.). After 1 hour, the cells were washed with 0.1 mL LTCC wash buffer and resuspended in 0.05 mL LTCC assay buffer (same composition as LTCC wash buffer). Plates were then transferred to a FLIPR⁹⁶ for assay. The FLIPR measured basal Fluo-4 fluorescence for 15 seconds, then added 0.05 mL of each compound tested diluted in LTCC assay buffer at final concentrations ranging from about 846 pM to about 17 μM. Fluo-4 fluorescence was then measured for 5 minutes. 0.1 mL KCl agonist dissolved in LTCC assay buffer was then added to the cells to produce a final concentration of 90 mM KCl, and fluorescence was measured for another 45 seconds. Data were collected over the entire time course and analyzed using Excel, Graph Pad Prism, or an in-house regression analysis software.

Alternative FLIPR Calcium Mobilization Assay for L-Type Calcium Channel.

Alternatively, the following cell line and procedure may be used for the FLIPR calcium mobilization assay for L-type calcium channel. One day prior to performing this assay, differentiated A7r5 cells are trypsinized, then seeded on tissue culture treated 96-well clear-bottom black plates (Becton Dickinson, Franklin Lakes, N.J.) at a dilution of 1:1 from a confluent T150 cm² flask. On the day of the assay, the plates are washed with A7r5 wash buffer (127 mM NaCl, 2 mM MgCl₂, 700 μM NaH₂PO₄, 5 mM CaCl₂, 5 mM NaHCO₃, 8 mM HEPES, 10 mM glucose, pH 7.4), then loaded with 0.1 mL of A7r5 wash buffer containing Fluo-4 (3 μM final concentration, Molecular Probes, Eugene, Oreg.). After 1 hour, the cells are washed with 0.1 mL A7r5 wash buffer and resuspended in 0.05 mL A7r5 assay buffer that is composed of A7r5 wash buffer plus 50 μM valinomycin (Sigma). Plates are then transferred to a FLIPR⁹⁶ for assay. The FLIPR measures basal Fluo-4 fluorescence for 15 seconds, then adds 0.05 mL of each compound tested diluted in A7r5 assay buffer at final concentrations ranging from about 846 pM to about 17 μM. Fluo-4 fluorescence is then measured for 5 minutes. 0.1 mL KCl agonist dissolved in A7r5 assay buffer is then added to the cells to produce a final concentration of 90 mM KCl, and fluorescence was measured for another 45 seconds. Data were collected over the entire time course and analyzed using Excel, Graph Pad Prism, or an in-house regression analysis software.

Cloning of N- and L-type calcium channel subunit open reading frame cDNAs. Five cDNAs encoding subunits of the rat N- or L-type calcium channels were cloned by PCR amplification in order to reconstitute functional channels in a heterologous system. These were the alpha1b (α1b), beta1 (β1), beta3 (β3), alpha2delta (α2δ), and alpha1c (α1c) subunit cDNAs. The alpha1b subunit cDNA has been described by Dubel et al. in Proc. Natl. Acad. Sci. U.S.A 89: 5058-5062 (1992). The beta1 subunit cDNA has been described by Pragnell et al. in FEBS Lett. 291: 253-258 (1991). The beta3 subunit cDNA has been described by Castellano et al. in J. Biol. Chem. 268: 12359-12366 (1993). The alpha2delta subunit cDNA has been described by Kim et al. in Proc. Natl. Acad. Sci. U.S.A. 89: 3251-3255 (1992). The alpha1c subunit cDNA has been described by Koch et al. in J. Biol. Chem. 265: 17786-17791 (1990).

The 7.0 kb cDNA containing the entire α1b open reading frame (ORF) was PCR amplified as two overlapping cDNA fragments, i.e., a 2.7 kb 5′ fragment and a 4.4 kb 3′ fragment. The 5′ fragment was amplified from rat brain cDNA using primers 1 (SEQ ID NO:1, TABLE 1) and 2 (SEQ ID NO:2, TABLE 1), and the 3′ fragment was amplified from rat spinal cord cDNA using primers 3 (SEQ ID NO:3, TABLE 1) and 4 (SEQ ID NO:4, TABLE 1). The two fragments were joined by ligation at a common restriction site to create the entire 7.0 kb cDNA. This ORF encodes the protein isoform generated by alternative splicing termed “+A ΔSFMG ΔET” according to the nomenclature of Lin et al. (Neuron 18: 153-166 (1997)). The entire cDNA was sequenced with redundant coverage on both strands. The cDNA was then inserted into the mammalian expression vector pcDNA6.2DEST (Invitrogen, Carlsbad Calif.) by homologous recombination using the Gateway system (Invitrogen).

The 1.8 kb cDNA encoding the β1 subunit, the 1.45 cDNA encoding the beta3 subunit, and the 3.3 kb cDNA encoding the alpha2delta subunit were cloned by PCR amplification from rat spinal cord cDNA (β1) or brain cDNA (β3, α2δ). Primers 5 (SEQ ID NO:5, TABLE 1) and 6 (SEQ ID NO:6, TABLE 1) were used for the β1 cDNA amplification; primers 7 (SEQ ID NO:7, TABLE 1) and 8 (SEQ ID NO:8, TABLE 1) were used for the β3 cDNA amplification; and primers 9 (SEQ ID NO:9, TABLE 1) and 10 (SEQ ID NO:10, TABLE 1) were used for the α2δ cDNA amplification. PCR products were subcloned and fully sequenced on both strands. Clones matching the reference sequence (β1: NM_{—}017346; β3: NM_{—}012828; α2δ: M86621) and the gene's GenBank rat genomic DNA sequences were recombined into the mammalian expression vector pcDNA3.2DEST (β1, β3) or pcDNA3.1-Zeo (α2δ), which had been modified to a vector compatible with the Gateway recombination system using the Gateway vector adaptor kit (Invitrogen). Proper recombination was confirmed by sequencing of recombinogenic regions. For β3 expression vector, proper protein expression was confirmed by Western blot analysis of lysates of transfected HEK293 cells using a rabbit polyclonal antiserum directed against the rat β3 subunit (USA Biological).

The 6.5 kb cDNA encoding the L-type calcium channel α1c subunit was cloned by PCR amplification from rat heart cDNA using primers 11 (SEQ ID NO: 11, TABLE 1) and 12 (SEQ ID NO:12, TABLE 1). The PCR fragment was subcloned and fully sequenced on both strands to confirm its identity. A clone matching consensus reference sequence M59786 and rat genomic DNA sequences was recombined into the mammalian expression vector pcDNA6.2DEST. Sequences around the recombinogenic region were sequenced to confirm accurate recombination into the expression vector.

TABLE 1

| | SEQ ID |
|---|---|
| PRIMER SEQUENCE | NO. |
| CACC ATG GTC CGC TTC GGG GAC | 1 |
| CCG TTC AGT GGC CTC CTC C | 2 |
| C TAG CAC CAG TGA TCC TGG TCTG | 3 |
| AGT GCG TTG TGA GCG CAG TA | 4 |
| CAC CAT GGT CCA GAA GAG CGG | 5 |
| TCTCAGCGGATGTAGACGCCT | 6 |
| CAC CAT GTA TGA CGA CTC CTA C | 7 |
| GGT GGT CAG TAG CTG TCC TTA GG | 8 |
| CAC CAT GGC TGC TGG CTG CCT | 9 |
| AGA GGG TCA CCA TAG ATA GTG TCT G | 10 |
| CACCATGATTCGGGCCTTCGCT | 11 |
| AGCCTGCGGACTACAGGTTGCTGAC | 12 |

N-type Recombinant Cell Line Development. N-type calcium channel expressing HEK-293 cells were created in two stages. Stage 1 was created as follows. The rat α1b, and β3 cDNA expression constructs (2.5 μg each) were co-transfected into human embryonic kidney (HEK-293) cells by Lipofectamine Plus reagent (Invitrogen), as per manufacturer's instructions. 24 hours later, cells were split in limiting dilution into multiple 96-well plates in selection media containing 20 μg/mL blasticidin and 500 μg/mL geneticin, and incubated for 3 weeks at 37° C., 5% CO₂, 95% humidity. Plates containing ≦1 clone per well were cultured until wells positive for single clones were confluent. Individual clones were then arrayed into columns of a destination 96-well plate, and partly split into 6-well plates for culture maintenance. Array plates were washed once with IMR32 buffer and cells loaded for 1 hour with 0.1 mL of IMR32 buffer containing Fluo-4 (3 μM final concentration, Molecular Probes). Then they were washed twice with 0.1 mL of IMR32 buffer, and replaced with 0.1 mL IMR32 buffer. Plates were then transferred to a FLIPR⁹⁶ for assay. The FLIPR they were washed twice with 0.1 mL of IMR32 buffer, and replaced with 0.1 mL dissolved in IMR32 buffer and measured fluorescence for another 45 seconds. Final KCl concentration was 90 mM. Data were collected over the entire time course and analyzed using Excel, Graph Pad Prism, or Activity Base (version 5.1, IDBS, Parsippany, N.J.) software. The clone with the greatest signal-to-noise ratio, best stability of response with passage number, and best adhesion to PDL precoated plates (Becton Dickinson) was expanded, characterized and used for stage 2 cell line development.

Stage 2 of N-type cell line development was carried out as follows. The rat α2δ cDNA expression construct (5 μg each) was transfected into the stage 1 N-type clonal cell line by Lipofectamine Plus reagent (Invitrogen), as per manufacturer's instructions. 24 hours later, cells were split in limiting dilution into multiple 96-well plates in selection media containing 20 μg/mL blasticidin, 500 μg/mL geneticin, and 250 μg/mL zeocin and incubated for 3 weeks at 37° C., 5% CO₂, 95% humidity. Plates containing ≦1 clone per well were cultured and handled according to the same steps and procedures described above for the stage 1 cell line. The three clones with the greatest signal-to-noise, best stability of response with passage number, and best adhesion to PDL precoated plates (Becton Dickinson) were expanded, characterized and tested in electrophysiology for the best current size, N-type pharmacology, N-type characteristic current-voltage relationship and kinetics as described below.

L-type Recombinant Cell Line Development. L-type calcium channel expressing HEK-293 cells were created in two stages. Stage 1 was created as follows. The rat α1c, and β1 cDNA expression constructs (2.5 μg each) were co-transfected into human embryonic kidney (HEK-293) cells by Lipofectamine Plus reagent (Invitrogen), as per manufacturer's instructions. 24 hours later, cells were split in limiting dilution into multiple 96-well plates in selection media containing 20 μg/mL blasticidin and 500 μg/mL geneticin, and incubated for 3 weeks at 37° C., 5% CO₂, 95% humidity. Plates containing ≦1 clone per well were cultured until wells positive for single clones were confluent. Individual clones were then arrayed into columns of a destination 96-well plate, and partly split into 6-well plates for culture maintenance. Array plates were washed once with LTCC wash (or assay) buffer and cells loaded for 1 hour with 0.1 mL of LTCC buffer containing Fluo-4 (3 μM final concentration, Molecular Probes). Then they were washed twice with 0.1 mL of LTCC buffer, and replaced with 0.1 mL LTCC buffer. Plates were then transferred to a FLIPR⁹⁶ for assay. The FLIPR measured basal Fluo-4 fluorescence for 315 seconds, then added 0.1 mL KCl agonist dissolved in LTCC buffer and measured fluorescence for another 45 seconds. Final KCl concentration was 90 mM. Data were collected over the entire time course and analyzed using Excel, Graph Pad Prism, or Activity Base software. The clone with the greatest signal-to-noise ratio, best stability of response with passage number, and best adhesion to PDL precoated plates (Becton Dickinson) was expanded, characterized and used for stage 2 cell line development.

Stage 2 of L-type cell line development was carried out as follows. The rat α2δ cDNA expression construct (5 μg each) was transfected into the stage 1 L-type clonal cell line by Lipofectamine Plus reagent (Invitrogen), as per manufacturer's instructions. 24 hours later, cells were split in limiting dilution into multiple 96-well plates in selection media containing 20 μg/mL blasticidin, 500 μg/mL geneticin, and 250 μg/mL zeocin and incubated for 3 weeks at 37° C., 5% CO₂, 95% humidity. Plates containing ≦1 clone per well were cultured and handled according to the same steps and procedures described above for the stage 1 cell line. The three clones with the greatest signal-to-noise, best stability of response with passage number, and best adhesion to PDL precoated plates (Becton Dickinson) were expanded and characterized.

N-type Electrophysiology in Recombinant Cells. For electrophysiological recording, the cells expressing α1b, β3 and α2δ subunits were seeded on 35-mm culture Petri dishes at a density of approximately 10⁴ cells/dish and kept in an incubator for up to three days for subsequent recordings. For recordings, the dishes were positioned on the stage of an inverted microscope (Nikon, Eclipse E600, Japan) and superfused with a bath solution comprised of BaCl₂ (11 mM), MgCl₂ (1.5 mM), HEPES (10 mM), TEA chloride (120 mM), glucose (10 mM) adjusted to pH 7.4 with KOH. Whole-cell voltage-clamp recordings were made using conventional patch-clamp techniques (Hamill et al., Pfluegers Arch. 391: 85-100 (1981)) at room temperature (22-24° C.). The patch-clamp pipettes were pulled from WPI, thick-walled borosilicate glass (WPI, Sarasota, Fla.). Currents were recorded using an Axopatch 200A amplifier (Axon Instruments, Union City, Calif.) and were leak-subtracted (P/4), low-pass filtered (1 kHz, 4-pole Bessel), digitized (20-50-μs intervals), and stored using Digidata 1200 B interface and Pclamp8.0/Clampex software (Axon Instruments, Union City, Calif.). The pipettes were back-filled with internal solution containing CsCl (110 mM), MgCl₂ (3 mM), EGTA (3 mM), HEPES (40 mM), Mg-ATP (4 mM), Na₂GTP (0.5 mM), and adjusted to pH 7.2 with CsOH. The pipette resistance ranged from 2 to 3 MOhm and was compensated by 75-80% by the built-in electronic circuitry.

Currents were elicited by stepping from a holding potential of −90 mV to 0 mV for 20 ms every 20 sec. At the −90 mV membrane voltage about 50% of channels were in the inactivated state, and thus contact with a blocker would involve interaction with both resting and inactivated channels. Every drug was applied at 3 to 4 concentrations increasing in a cumulative manner. Fractional inhibition levels in steady-state were used to draw the partial inhibition concentration curves to get the IC₅₀ (i.e. concentration causing 50% reduction in the size of the response) values at −90 mV. Stock solutions of each test compound were prepared using DMSO. Serial dilutions to desired concentrations were done with bath solution; concentration of DMSO in final solutions was 0.1%. Drugs were applied by gravity flow using a plane multi-barrel array shooter positioned 0.5 mm apart from the cell.

All curve fittings were carried out using Origin software (version 5.0, Microcal). A Hill equation was fit to the concentration-inhibition curves to determine IC₅₀ values.

N-type Electrophysiology in Neuronal Cells. To determine dissociation constants in resting versus inactivated state for N-type calcium channels, neuronal cells that endogenously express N-type calcium channels can be used. For electrophysiological recording, the neuronal cells expressing N-type calcium channels are seeded on 35-mm culture Petri dishes at a density of approximately 10⁴ cells/dish and kept in an incubator for up to three days for subsequent recordings. For recordings, the dishes are positioned on the stage of an inverted microscope (Nikon, Eclipse E600, Japan) and superfused with a bath solution comprised of BaCl₂ (11 mM), MgCl₂ (1.5 mM), HEPES (10 mM), TEA chloride (120 mM), glucose (10 mM) adjusted to pH 7.4 with KOH. Whole-cell voltage-clamp recordings are made using conventional patch-clamp techniques (Hamill et al., Pfluegers Arch. 391: 85-100 (1981)) at room temperature (22-24° C.). The patch-clamp pipettes are pulled from WPI, thick-walled borosilicate glass (WPI, Sarasota, Fla.). Currents are recorded using an Axopatch 200A amplifier (Axon Instruments, Union City, Calif.) and leak-subtracted (P/4), low-pass filtered (1 kHz, 4-pole Bessel), digitized (20-50-μs intervals), and stored using Digidata 1200 B interface and Pclamp8.0/Clampex software (Axon Instruments, Union City, Calif.). The pipettes are back-filled with internal solution containing CsCl (1 10 mM), MgCl₂ (3 mM), EGTA (3 mM), HEPES (40 mM), Mg-ATP (4 mM), Na₂GTP (0.5 mM), and adjusted to pH 7.2 with CsOH. The pipette resistance ranges from 2 to 3 MOhm and is compensated by 75-80% by the built-in electronic circuitry.

Currents are elicited by stepping from a holding potential of −90 mV to 0 mV for 20 ms every 10 sec. At the −90 mV membrane voltage a proportion of channels is in the inactivated state, and thus contact with a blocker would involve interaction with both resting and inactivated channels. This protocol is used as a first tier screen. For dissection of two components of inhibition (resting block with the apparent dissociation constant Kᵣ and inactivated state block with Kᵢ), steady-state inactivation curves are collected using a double-pulse protocol. Three-second long depolarizing pre-pulse incrementing in 10 mV steps is followed by a 10 ms test pulse to 0 mV. Stock solutions of each test compound are prepared using DMSO. Serial dilutions to desired concentrations are done with bath solution; concentration of DMSO in final solutions is 0.1%. Drugs are applied by gravity flow using a plane multi-barrel array shooter positioned ˜1 mm apart from the cell.

All curve fittings can be carried out using Origin software (version 5.0, Microcal). A Hill equation is used to fit the concentration-response curves and to determine IC₅₀ values. A Boltzman equation is used to fit inactivation curves, returning half-inactivation voltage, V_{0.5}, slope p and the amplitude of current at the most negative voltage where eventually all channels are in the resting state. These parameters are used to calculate the apparent dissociation constants: Kᵣ=((Ab/Ac)/(1−(Ab/Ac))*[b]) where [b] is the drug concentration, Ac is the maximum test current amplitude in control conditions and Ab is the maximum test current amplitude in the presence of a blocker; Kᵢ=[b]/((exp(−(dx/p))*(1+([b]/Kᵣ))−1) where dx is the difference between half-inactivation voltage V_{0.5} in the presence and absence of drug and p is the slope.

In Vivo Pharmacology

The compounds of the present invention can be tested for in vivo anticonvulsant activity after i.v., p.o., or i.p. injection using any of a number of anticonvulsant tests in mice, including the maximum electroshock seizure test (MES). Maximum electroshock seizures are induced in male NSA mice weighing between 15-20 g and in male Sprague-Dawley rats weighing between 200-225 g by application of current (for mice: 50 mA, 60 pulses/sec, 0.8 msec pulse width, 1 sec duration, D.C.; for rats: 99 mA, 125 pulses/sec, 0.8 msec pulse width, 2 sec duration, D.C.) using a Ugo Basile ECT device (Model 7801). Mice are restrained by gripping the loose skin on their dorsal surface and saline-coated corneal electrodes are held lightly against the two corneae. Rats are allowed free movement on the bench top and ear-clip electrodes are used. Current is applied and animals are observed for a period of up to 30 seconds for the occurrence of a tonic hindlimb extensor response. A tonic seizure is defined as a hindlimb extension in excess of 90 degrees from the plane of the body. Results can be treated in a quantal manner.

The compounds can be tested for their antinociceptive activity in the formalin model as described in Hunskaar, S., O. B. Fasmer, and K. Hole, J. Neurosci. Methods 14: 69-76 (1985). Male Swiss Webster NIH mice (20-30 g; Harlan, San Diego, Calif.) can be used in all experiments. Food is withdrawn on the day of experiment. Mice are placed in Plexiglass jars for at least 1 hour to acclimate to the environment. Following the acclimation period mice are weighed and given either the compound of interest administered i.p. or p.o., or the appropriate volume of vehicle (10% Tween-80) as control. Fifteen minutes after the i.p. dosing, and 30 minutes after the p.o. dosing mice are injected with formalin (20 μL of 5% formaldehyde solution in saline) into the dorsal surface of the right hind paw. Mice are transferred to the Plexiglass jars and monitored for the amount of time spent licking or biting the injected paw. Periods of licking and biting are recorded in 5-minute intervals for 1 hour after the formalin injection. All experiments are done in a blinded manner during the light cycle. The early phase of the formalin response is measured as licking/biting between 0-5 minutes, and the late phase is measured from 15-50 minutes. Differences between vehicle and drug treated groups can be analyzed by one-way analysis of variance (ANOVA). A P value<0.05 is considered significant. Compounds are considered to be efficacious for treating acute and chronic pain if they have activity in blocking both the early and second phase of formalin-induced paw-licking activity.

Compounds can be tested for their potential to treat chronic pain (i.e., antiallodynic and antihyperalgesic activities) using the Chung model of peripheral neuropathy (Kim and Chung, Pain 50: 355-363 (1992)). Male Sprague-Dawley rats weighing between 200-225 g are anesthetized with halothane (1-3% in a mixture of 70% air and 30% oxygen), and their body temperature controlled during anesthesia through use of a homeotherrnic blanket. A 2-cm dorsal midline incision is then made at the L5 and L6 level, and the para-vertebral muscle groups retracted bilaterally. L5 and L6 spinal nerves are then exposed, isolated, and tightly ligated with 6-0 or 7-0 silk suture. A sham operation is performed exposing the contralateral L5 and L6 spinal nerves, without ligating, as a negative control.

Tactile Allodynia: Sensitivity to non-noxious mechanical stimuli can be measured in animals to assess tactile allodynia. Rats are transferred to an elevated testing cage with a wire mesh floor and allowed to acclimate for five to ten minutes. A series of von Frey monofilaments are applied to the plantar surface of the hindpaw to determine the animal's withdrawal threshold. The first filament used possesses a buckling weight of 9.1 gms (0.96 log value) and is applied up to five times to see if it elicits a withdrawal response. If the animal has a withdrawal response, then the next lightest filament in the series would be applied up to five times to determine if it also could elicit a response. This procedure is repeated with subsequent lesser filaments until there is no response and the identity of the lightest filament that elicits a response is recorded. If the animal does not have a withdrawal response from the initial 9.1 gms filament, then subsequent filaments of increased weight are applied until a filament elicits a response and the identity of this filament is recorded. For each animal, three measurements are made at every time point to produce an average withdrawal threshold determination. Tests can be performed prior to, and at 1, 2, 4 and 24 hours post drug administration.

Mechanical Hyperalgesia: Sensitivity to noxious mechanical stimuli can be measured in animals using the paw pressure test to assess mechanical hyperalgesia. In rats, hind paw withdrawal thresholds (“PWT”), measured in grams, in response to a noxious mechanical stimulus are determined using an analgesymeter (Model 7200, commercially available from Ugo Basile of Italy), as described in Stein (Biochemistry & Behavior 31: 451-455 (1988)). The rat's paw is placed on a small platform, and weight is applied in a graded manner up to a maximum of 250 grams. The endpoint is taken as the weight at which the paw is completely withdrawn. PWT is determined once for each rat at each time point. PWT can be measured only in the injured paw, or in both the injured and non-injured paw. In one non-limiting embodiment, mechanical hyperalgesia associated with nerve injury induced pain (neuropathic pain) can be assessed in rats. Rats are tested prior to surgery to determine a baseline, or normal, PWT. Rats are tested again 2 to 3 weeks post-surgery, prior to, and at different times after (e.g. 1, 3, 5 and 24 hr) drug administration. An increase in PWT following drug administration indicates that the test compound reduces mechanical hyperalgesia.

Pharmaceutical Compositions

Although a compound of the present invention may be administered to a mammal in the form of a raw chemical without any other components present, the compound is preferably administered as part of a pharmaceutical composition containing the compound combined with a suitable pharmaceutically acceptable carrier. Such a carrier can be selected from pharmaceutically acceptable excipients and auxiliaries. Compositions within the scope of the present invention include all compositions where a compound of the present invention is combined with a pharmaceutically acceptable carrier. In a preferred embodiment, the compound is present in the composition in an amount that is effective to achieve its intended therapeutic purpose. While individual needs may vary, a determination of optimal ranges of effective amounts of each compound is within the skill of the art. Typically, the compounds may be administered to mammal, e.g. human, orally at a dose of from about 0.0025 to about 1500 mg per kg body weight of the mammal, or an equivalent amount of a pharmaceutically acceptable salt thereof, per day to treat the particular disorder. A useful oral dose of a compound of the present invention administered to a mammal is from about 0.0025 to about 50 mg per kg body weight of the mammal, or an equivalent amount of the pharmaceutically acceptable salt thereof. For intramuscular injection, the dose is typically about one-half of the oral dose.

A unit oral dose may comprise from about 0.01 to about 50 mg, and preferably about 0.1 to about 10 mg, of the compound. The unit dose can be administered one or more times daily as one or more tablets, each containing from about 0.01 to about 50 mg of the compound, or an equivalent amount of a pharmaceutically acceptable salt or solvate thereof.

In one embodiment, a pharmaceutical composition of the present invention can be administered orally and is formulated into tablets, dragees, capsules or an oral liquid preparation.

Alternatively, a pharmaceutical composition of the present invention can be administered rectally, and is formulated in suppositories.

Alternatively, a pharmaceutical composition of the present invention can be administered by injection.

Alternatively, a pharmaceutical composition of the present invention can be administered transdermally.

Alternatively, a pharmaceutical composition of the present invention can be administered by inhalation or by intranasal administration.

Alternatively, a pharmaceutical composition of the present invention can be administered by the intravaginal route.

A pharmaceutical composition of the present invention can contain from about 0.01 to 99 percent by weight, and preferably from about 0.25 to 75 percent by weight, of active compound(s).

The present methods of the invention, such as the method for treating, preventing, or ameliorating a disorder responsive to the blockade of calcium channels in an animal in need thereof, can further comprise administering the second therapeutic agent to the animal being administered a compound of Formula I, I′, I″, II or II′. In one embodiment, the second therapeutic agent is administered in an effective amount. Effective amounts of the other therapeutic agents are known to those skilled in the art. However, it is well within the skilled artisan's purview to determine the other therapeutic agent's optimal effective-amount range. In one embodiment of the invention, where another therapeutic agent is administered to an animal, the effective amount of the compound of the present invention is less than its effective amount would be where the other therapeutic agent is not administered. In this case, without being bound by theory, it is believed that compounds of the present invention and the other therapeutic agent act synergistically to treat, prevent, or ameliorate a disorder or condition.

The second therapeutic agent can be, but is not limited to, an opioid agonist, a non-opioid analgesic, a non-steroidal anti-inflammatory agent, an antimigraine agent, a Cox-II inhibitor, a β-adrenergic blocker, an anticonvulsant, an antidepressant, an anticancer agent, an agent for treating addictive disorder, an agent for treating Parkinson's disease and parkinsonism, an agent for treating anxiety, an agent for treating epilepsy, an agent for treating a seizure, an agent for treating a stroke, an agent for treating a pruritic condition, an agent for treating psychosis, an agent for treating ALS, an agent for treating a cognitive disorder, an agent for treating a migraine, an agent for treating vomiting, an agent for treating dyskinesia, or an agent for treating depression, and mixtures thereof.

Examples of useful opioid agonists include, but are not limited to, alfentanil, allylprodine, alphaprodine, anileridine, benzylmorphine, bezitramide, buprenorphine, butorphanol, clonitazene, codeine, desomorphine, dextromorarmide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof.

In certain embodiments, the opioid agonist is selected from codeine, hydromorphone, hydrocodone, oxycodone, dihydrocodeine, dihydromorphine, morphine, tramadol, oxymorphone, pharmaceutically acceptable salts thereof, and mixtures thereof. Examples of useful non-opioid analgesics include non-steroidal anti-inflammatory agents, such as aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam, and pharmaceutically acceptable salts thereof, and mixtures thereof. Examples of other suitable non-opioid analgesics include the following, non limiting, chemical classes of analgesic, antipyretic, nonsteroidal antiinflammatory drugs: salicylic acid derivatives, including aspirin, sodium salicylate, choline magnesium trisalicylate, salsalate, diflunisal, salicylsalicylic acid, sulfasalazine, and olsalazin; para aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams piroxicam, tenoxicam), and pyrazolidinediones (phenylbutazone, oxyphenthartazone); and alkanones, including nabumetone. For a more detailed description of the NSAIDs, see Paul A. Insel, Analgesic Antipyretic and Antiinflammatory Agents and Drugs Employed in the Treatment of Gout, in Goodman & Gilman's The Pharmacological Basis of Therapeutics 617-57 (Perry B. Molinhoff and Raymond W. Ruddon eds., 9th ed 1996) and Glen R. Hanson, Analgesic, Antipyretic and Anti Inflammatory Drugs in Remington: The Science and Practice of Pharmacy Vol II 1196-1221 (A. R. Gennaro ed. 19th ed. 1995) which are hereby incorporated by reference in their entireties. Suitable Cox-II inhibitors and 5-lipoxygenase inhibitors, as well as combinations thereof, are described in U.S. Pat. No. 6,136,839, which is hereby incorporated by reference in its entirety. Examples of useful Cox II inhibitors include, but are not limited to, rofecoxib and celecoxib. Examples of useful antimigraine agents include, but are not limited to, alpiropride, bromocriptine, dihydroergotamine, dolasetron, ergocomine, ergocominine, ergocryptine, ergonovine, ergot, ergotamine, flumedroxone acetate, fonazine, ketanserin, lisuride, lomerizine, methylergonovine, methysergide, metoprolol, naratriptan, oxetorone, pizotyline, propranolol, risperidone, rizatriptan, sumatriptan, timolol, trazodone, zolmitriptan, and mixtures thereof.

Examples of useful β-adrenergic blockers include, but are not limited to, acebutolol, alprenolol, amosulabol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol, indenolol, labetalol, levobunolol, mepindolol, metipranolol, metoprolol, moprolol, nadolol, nadoxolol, nebivalol, nifenalol, nipradilol, oxprenolol, penbutolot, pindolol, practolol, pronethalol, propranolol, sotalol, sulfinalol, talinolol, tertatolol, tilisolol, timolol, toliprolol, and xibenolol.

Examples of useful anticonvulsants include, but are not limited to, acetylpheneturide, albutoin, aloxidone, aminoglutethimide, 4-amino-3-hydroxybutyric acid, atrolactamide, beclamide, buramate, calcium bromide, carbamazepine, cinromide, clomethiazole, clonazepam, decimemide, diethadione, dimethadione, doxenitroin, eterobarb, ethadione, ethosuximide, ethotoin, felbamate, fluoresone, gabapentin, 5-hydroxytryptophan, latnotrigine, magnesium bromide, magnesium sulfate, mephenytoin, mephobarbital, metharbital, methetoin, methsuximide, 5-methyl-5-(3-phenanthryl)-hydantoin, 3-methyl-5-phenylhydantoin, narcobarbital, nimetazepam, nitrazepam, oxcarbazepine, paramethadione, phenacemide, phenetharbital, pheneturide, phenobarbital, phensuximide, phenylmethylbarbituric acid, phenytoin, phethenylate sodium, potassium bromide, pregabaline, primidone, progabide, sodium bromide, solanum, strontium bromide, suclofenide, sulthiame, tetrantoin, tiagabine, topiramate, trimethadione, valproic acid, valpromide, vigabatrin, and zonisamide. Examples of useful antidepressants include, but are not limited to, binedaline, caroxazone, citalopram, (S)-citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

Examples of useful anticancer agents include, but are not limited to, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, and cisplatin.

Therapeutic agents useful for treating or preventing an addictive disorder include, but are not limited to, methadone, desipramine, arnantadine, fluoxetine, buprenorphine, an opiate agonist, 3-phenoxypyridine, or a serotonin antagonist.

Examples of useful therapeutic agents for treating or preventing Parkinson's disease and parkinsonism include, but are not limited to, carbidopa/levodopa, pergolide, bromocriptine, ropinirole, pramipexole, entacapone, tolcapone, selegiline, amantadine, and trihexyphenidyl hydrochloride.

Examples of useful therapeutic agents for treating or preventing anxiety include, but are not limited to, benzodiazepines, such as alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam, and triazolam; non-benzodiazepine agents, such as buspirone, gepirone, ipsapirone, tiospirone, zolpicone, zolpidem, and zaleplon; tranquilizers, such as barbituates, e.g., amobarbital, aprobarbital, butabarbital, butalbital, mephobarbital, methohexital, pentobarbital, phenobarbital, secobarbital, and thiopental; and propanediol carbamates, such as meprobamate and tybamate.

Examples of useful therapeutic agents for treating or preventing epilepsy or seizure include, but are not limited to, carbamazepine, ethosuximide, gabapentin, lamotrigine, phenobarbital, phenytoin, primidone, valproic acid, trimethadione, benzodiazepines, gamma-vinyl GABA, acetazolamide, and felbamate.

Examples of useful therapeutic agents for treating or preventing stroke include, but are not limited to, anticoagulants such as heparin, agents that break up clots such as streptokinase or tissue plasminogen activator, agents that reduce swelling such as mannitol or corticosteroids, and acetylsalicylic acid.

Examples of useful therapeutic agents for treating or preventing a pruritic condition include, but are not limited to, naltrexone; nalmefene; danazol; tricyclics such as amitriptyline, imipramine, and doxepin; antidepressants such as those given below; menthol; camphor; phenol; pramoxine; capsaicin; tar; steroids; and antihistamines.

Examples of useful therapeutic agents for treating or preventing psychosis include, but are not limited to, phenothiazines such as chlorpromazine hydrochloride, mesoridazine besylate, and thoridazine hydrochloride; thioxanthenes such as chloroprothixene and thiothixene hydrochloride; clozapine; risperidone; olanzapine; quetiapine; quetiapine fumarate; haloperidol; haloperidol decanoate; loxapine succinate; molindone hydrochloride; pimozide; and ziprasidone.

Examples of useful therapeutic agents for treating or preventing ALS include, but are not limited to, baclofen, neurotrophic factors, riluzole, tizanidine, benzodiazepines such as clonazepan and dantrolene.

Examples of useful therapeutic agents for treating or preventing cognitive disorders include, but are not limited to, agents for treating or preventing dementia such as tacrine; donepezil; ibuprofen; antipsychotic drugs such as thioridazine and haloperidol; and antidepressant drugs such as those given below.

Examples of useful therapeutic agents for treating or preventing a migraine include, but are not limited to, sumatriptan; methysergide; ergotamine; caffeine; and beta-blockers such as propranolol, verapamil, and divalproex.

Examples of useful therapeutic agents for treating or preventing vomiting include, but are not limited to, 5-HT3 receptor antagonists such as odansetron, dolasetron, granisetron, and tropisetron; dopanine receptor antagonists such as prochlorperazine, thiethylperazine, chlorpromazine, metoclopramide, and domperidone; glucocorticoids such as dexamethasone; and benzodiazepines such as lorazepam and alprazolam.

Examples of useful therapeutic agents for treating or preventing dyskinesia include, but are not limited to, reserpine and tetrabenazine.

Examples of useful therapeutic agents for treating or preventing depression include, but are not limited to, tricyclic antidepressants such as amitryptyline, amoxapine, bupropion, clomipramine, desipramine, doxepin, imipramine, maprotiline, nefazadone, nortriptyline, protriptyline, trazodone, trimipramine, and venlafaxine; selective serotonin reuptake inhibitors such as citalopramn, (S)-citalopram, fluoxetine, fluvoxamine, paroxetine, and setraline; monoamine oxidase inhibitors such as isocarboxazid, pargyline, phenelzine, and tranylcypromine; and psychostimulants such as dextroamphetamine and methylphenidate.

A compound of the present invention and the second therapeutic agent can act additively or, in one embodiment, synergistically. In one embodiment, a compound of the present invention is administered concurrently with the second therapeutic agent; for example, a composition comprising an effective amount of a compound of Formula I, I′, I″, II or II′, and an effective amount of the second therapeutic agent can be administered. Alternatively, a composition comprising an effective amount of a compound of Formula I, I′, I″, II or II′ and a different composition comprising an effective amount of the second therapeutic agent can be concurrently administered. In another embodiment, an effective amount of a compound of the present invention is administered prior or subsequent to administration of an effective amount of the second therapeutic agent. In this embodiment, the compound of the present invention is administered while the second therapeutic agent exerts its therapeutic effect, or the other therapeutic agent is administered while the compound of the present invention exerts its preventive or therapeutic effect for treating, ameliorating or preventing a disorder or condition.

A pharmaceutical composition of the present invention can be administered to any animal that may experience the beneficial effects of a compound of the present invention. Foremost among such animals are mammals, e.g., humans and companion animals, although the invention is not intended to be so limited.

A pharmaceutical composition of the present invention can be administered by any means that achieves its intended purpose. For example, administration can be by the parenteral, subcutaneous, intravenous, intramuscular, intraperitoneal, transdermal, intranasal, rectal, intravaginal or buccal route, or by inhalation. Alternatively, or concurrently, administration can be by the oral route. The dosage administered and route of administration will vary, depending upon the circumstances of the particular subject, and taking into account such factors as age, health, and weight of the recipient, condition or disorder to be treated, kind of concurrent treatment, if any, frequency of treatment, and the nature of the effect desired.

A pharmaceutical composition of the present invention is preferably manufactured in a manner which is itself known, for example, by means of conventional mixing, granulating, dragee-making, dissolving, extrusion, or lyophilizing processes. Thus, pharmaceutical compositions for oral use can be obtained by combining the active compound with solid excipients, optionally grinding the resulting mixture and processing the mixture of granules, after adding suitable auxiliaries, if desired or necessary, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate can be used. Dye stuffs or pigments may be added to the tablets or dragee coatings, for example, for identification or in order to characterize combinations of active compound doses.

Examples of other pharmaceutical preparations that can be used orally include push-fit capsules made of gelatin, or soft, sealed capsules made of gelatin and a plasticizer such as glycerol or sorbitol. The push-fit capsules can contain a compound in the form of granules, which may be mixed with fillers such as lactose, binders such as starches, and/or lubricants such as talc or magnesium stearate and, optionally, stabilizers, or in the form of extruded multiparticulates. In soft capsules, the active compounds are preferably dissolved or suspended in suitable liquids, such as fatty oils or liquid paraffin. In addition, stabilizers may be added.

Possible pharmaceutical preparations for rectal administration include, for example, suppositories, which consist of a combination of one or more active compounds with a suppository base. Suitable suppository bases include natural and synthetic triglycerides, and paraffin hydrocarbons, among others. It is also possible to use gelatin rectal capsules consisting of a combination of active compound with a base material such as, for example, a liquid triglyceride, polyethylene glycol, or paraffin hydrocarbon.

Suitable formulations for parenteral administration include aqueous solutions of the active compound in a water-soluble form such as, for example, a water-soluble salt, alkaline solution, or acidic solution. Alternatively, a suspension of the active compound may be prepared as an oily suspension. Suitable lipophilic solvents or vehicles for such as suspension may include fatty oils (for example, sesame oil), synthetic fatty acid esters (for example, ethyl oleate), triglycerides, or a polyethylene glycol such as polyethylene glycol-400 (PEG-400). An aqueous suspension may contain one or more substances to increase the viscosity of the suspension, including, for example, sodium carboxymethyl cellulose, sorbitol, and/or dextran. The suspension may optionally contain stabilizers.

The following examples are illustrative, but not limiting, of the compounds, compositions and methods of the present invention. Suitable modifications and adaptations of the variety of conditions and parameters normally encountered in clinical therapy and which are obvious to those skilled in the art in view of this disclosure are within the spirit and scope of the invention.

In the following examples,

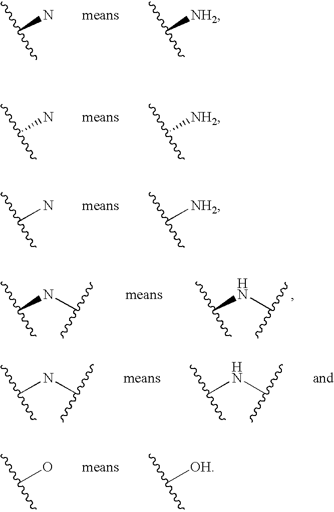

### Examples

### Example A1

### O-Pyridin-2-ylmethylhydroxylamine dihydrochloride

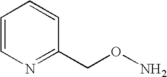

a) 2-Pyridiylmethanol (2.23 g, 20.0 mmol), N-hydroxyphthalimide (3.26 g, 20.0 mmol) and triphenylphosphine (5.35 g, 20.0 mmol) were dissolved in tetrahydrofuran (100 ml). Diethyl azodicarboxylate (40% in toluene, 10 ml, 22.0 mmol) was added to this mixture, and stirred at room temperature for 3 hours. The solvent was evaporated under reduced pressure and the residue was suspended in ethanol (40 ml). The resulting precipitate was collected and dried to give 2-(pyridin-2-ylmethoxy)isoindole-1,3-dione (3.60 g, 71%).

b) 2-(Pyridin-2-ylmethoxy)isoindole-1,3-dione (2.03 g, 8.00 mmol) and hydrazine monohydrate (400 mg, 8.00 mmol) were dissolved in ethanol (15 ml) and the mixture was refluxed for 1 hour. The solvent was evaporated under reduced pressure. The residue was purified by column chromatography on silica (ethyl acetate/methanol, 100/0-90/10) to give O-pyridin-2-ylmethylhydroxylamine as pale yellow oil. The oil was dissolved in ethyl acetate (15 ml) and methanol (10 ml), and then 4N—HCl in ethyl acetate (20 ml) was added to this solution. The solvent was evaporated under reduced pressure and the residue was suspended in hexane (30 ml). The resulting precipitate was collected and dried to give the title compound (1.37 g, 87%).

### Example A2

### O-(2-Chloropyridin-4-ylmethyl hydroxylamine

was prepared from (2-chloropyridin-4-yl)methanol as described in EXAMPLE A1.

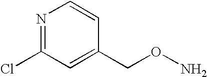

### Example A3

### 5-(Aminooxymethyl)-2-fluorobenzonitrile

was prepared from 2-fluoro-5-(hydroxymethyl)benzonitrile as described in EXAMPLE A1.

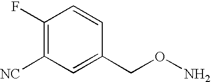

### Example A4

### O-((6-Methoxypyridin-2-yl)methyl)hydroxylamine

was prepared from (6-methoxypyridin-2-yl)methanol as described in EXAMPLE A1.

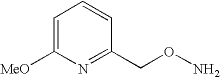

### Example A5

### O-((2-Methoxpyridin-3-yl)methyl)hydroxylamine

was prepared from (2-methoxypyridin-3-yl)methanol as described in EXAMPLE A1.

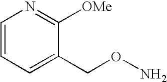

### Example A6

### O-((5-Fluoropyridin-2-yl)methyl)hydroxylamine

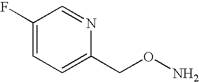

a) The mixture of 2-bromo-5-fluoropyridine (1.00 g, 0.5.68 mmol), palladium(II) acetate (127 mg, 0.568 mmol), 1,1′-bis(diphenylphosphino)ferrocene (630 mg, 1.14 mmol) and triethylamine (1.6 mmol) in ethano/N,N-dimethylformamide (1:1, 30 ml) was stirred under carbon monoxide atmosphere (1 atm) at 50° C. for 3 hours. H2O (20 ml) was added and the whole was filtrated through a pad of Celite. The filtrate was extracted with ethyl acetate, washed with H₂O and brine, dried (MgSO₄) and evaporated under reduced pressure. The residue was purified by column chromatography on silica (ethyl acetate/hexane, 20/80-50/50) to give ethyl 5-fluoropicolinate (754 mg, 78%, colorless solid).

b) To a solution of ethyl 5-fluoropicolinate (377 mg, 2.23 mmol).in tetrahydrofuran (6 ml), LiBH₄ (73 mg, 3.34 mmol) was added at 0° C. and stirred at room temperature for 1 hour. The reaction was quenched with ice-water and extracted with ethyl acetate, dried (Na₂SO₄) and evaporated under reduced pressure. The residue was purified by column chromatography on silica (ethyl acetate/hexane, 20/80-60/40) to give (5-fluoropyridin-2-yl)methanol (309 mg, 54%, colorless oil).

c) O-((5-Fluoropyridin-2-yl)methyl)hydroxylamine was prepared from (5-fluoropyridin-2-yl)methanol as described in EXAMPLE A1.

### Example A7

### O-((3-Fluoropyridin-2-yl)methyl)hydroxylamine

was prepared from (3-fluoropyridin-2-yl)methanol as described in EXAMPLE A1.

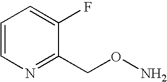

### Example A8

### O-((5-Chloropyridin-2-yl)methyl)hydroxylamine

was prepared from (5-chloropyridin-2-yl)methanol as described in EXAMPLE A1.

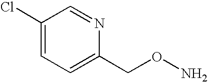

### Example A9

### 3-(Aminooxymethyl)-N,N-dimethylaniline

was prepared from (3-(dimethylamino)phenyl)methanol as described in EXAMPLE A1.

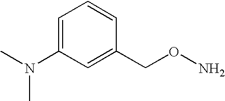

### Example A10

### O-((6-Methoxypyridin-3-yl)methyl)hydroxylamine

was prepared from (6-methoxypyridin-3-yl)methanol as described in EXAMPLE A1.

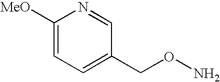

### Example A11

### O-((2-chloro-6-methoxypyridin-4-yl)methyl)hydroxylamine

was prepared from (2-chloro-6-methoxypyridin-4-yl)methanol as described in EXAMPLE A1.

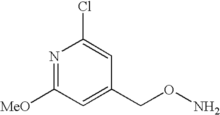

### Example A12

### O-((2-Methoxypyridin-4-yl)methyl)hydroxylamine

was prepared from (2-methoxypyridin-4-yl)methanol as described in EXAMPLE A1.

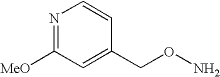

### Example A13

### (R)-tert-butyl 3-(aminooxymethyl)morpholine-4-carboxylate

was prepared from (S)-tert-butyl 3-(hydroxymethyl)morpholine-4-carboxylate as described in EXAMPLE A1.

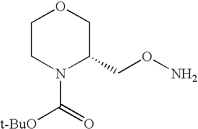

### Example A14

### (S)-tert-butyl 3-(aminooxymethyl)morpholine-4-carboxylate

was prepared from (R)-tert-butyl 3-(hydroxymethyl)morpholine-4-carboxylate as described in EXAMPLE A1.

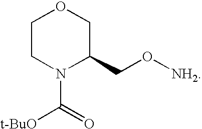

### Example A15

### 2-(Aminooxymethyl)isonicotinonitrile

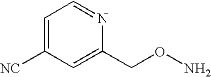

a) Sulfuric acid (1.5 ml) was added to the solution of 4-cyanopyridine (15.7 g, 151 mmol) in methanol (225 ml) at room temperature and refluxed for 30 minutes. A solution of ammonium peroxodisulfate (54.9 g, 241 mmol) in H₂O (100 ml) was slowly added over 30 minutes under reflux and the mixture was refluxed with stirring for 1 hour. After cooling, precipitated materials were filtered off and methanol was removed from the filtrate under reduced pressure. The residue was neutralized (pH=9) by adding saturated aqueous K₂CO₃ solution and precipitated materials were filtered off again. The filtrate was extracted with chloroform (100 ml×4), dried (Na₂SO4) and evaporated under reduced pressure. The residue was purified by column chromatography on silica (chloroform/methanol) to give 2-(hydroxymethyl)isonicotinonitrile (7.95 g, 39%, off-white solid).

b) 2-(aminooxymethyl)isonicotinonitrile was prepared from 2-(hydroxymethyl)isonicotinonitrile as described in EXAMPLE A1.

### Example A16

### O-((4-Methoxypyridin-2-yl)methyl)hydroxylamine

was prepared from (4-methoxypyridin-2-yl)methanol as described in EXAMPLE A1.

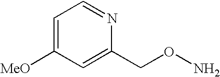

### Example A17

### O-(Isoquinolin-1-yl)hydroxylamine

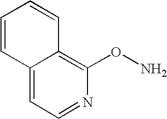

a) To a solution of potassium tert-butoxide (1.35 g, 12.0 mmol) in N,N-dimethylformamide (3 ml), a solution of ethyl acetohydroxamate (1.24 g, 12.0 mmol) in N,N-dimethylformamide (3 ml) was added and stirred at room temperature for 30 minutes. A solution of 1-chloroisoquinoline (1.72 g, 10.0 mmol) in N,N-dimethylformamide (4 ml) was added to the reaction mixture and stirred at room temperature for 2.5 hours. N,N-dimethylformamide was evaporated under reduced pressure and the residue was diluted with ethyl acetate (200 ml), washed with H₂O (50 ml), dried (MgSO4) and evaporated under reduced pressure. The residue was purified by column chromatography on silica (ethyl acetate/hexane, 20/80-40/60) to give (E)-ethyl N-isoquinolin-1-yloxyacetimidate (2.04 g, 89%, pale-yellow oil).

b) To a solution of (E)-ethyl N-isoquinolin-1-yloxyacetimidate (2.00 g, 8.68 mmol) in methanol/H₂O (3:1, 20 ml), sulfuric acid (1.85 ml) was added dropwise over 5 minutes and stirred at room temperature for 5 hours. H₂O (30 ml) was added and neutralized (pH=9) by adding saturated aqueous K₂CO₃ solution and precipitated materials were filtered off. The filtrate was diluted with ethyl acetate (100 ml), washed with H₂O (40 ml) and brine, dried (MgSO4) and evaporated under reduced pressure. The residue was purified by column chromatography on silica (ethyl acetate/hexane, 30/70) to give the title compound (607 mg, 32%, pale-yellow oil).

### Example A18

### 3-(Aminooxy(cyclopropyl)methyl)benzonitrile

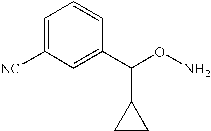

a) To a solution of 3-cyanobenzonitrile (1.33 g, 10.0 mmol) in tetrahydrofuran (50 ml), cyclopropylmagnesium bromide (0.5 M solution in tetrahydrofuran, 50 ml, 25 mmol) was added dropwise at −78° C. over 15 minutes and stirred at −78° C. for 12 hours. The reaction was quenched with HCl (1 M, 50 ml), extracted with ethyl acetate, washed with brine, dried (MgSO₄) and evaporated under reduced pressure. The residue was purified by column chromatography on silica (ethyl acetate/hexane, 20/80-40/60) to give 3-(cyclopropyl(hydroxy)methyl)benzonitrile (1.08 g, 60%, pale-yellow oil).

c) 3-(Aminooxy(cyclopropyl)methyl)benzonitrile was prepared from 3-(cyclopropyl(hydroxy)methyl)benzonitrile as described in EXAMPLE A1.

### Example A19

### O-((4-Methoxyquinolin-2-yl)methyl)hydroxylamine

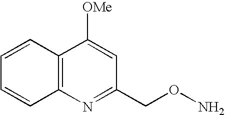

a) To a solution of 4-methoxy-2-quinolinecarboxylic acid (2.14 g, 10.0 mmol) and K₂CO₃ (1.66 g, 12.0 mmol) in acetonitrile (40 ml) and N,N-dimethylformamide (50 ml), iodomethane (2.25 ml, 3.6 mmol) was added and stirred at at 80° C. for 4 hours. The solvents were evaporated under reduced pressure and the residue was diluted with ethyl acetate (200 ml), washed with H₂O (50 ml) and brine, dried (MgSO₄) and evaporated under reduced pressure. The residual solid was triturated with ethyl acetate/hexane to give methyl 4-methoxyquinoline-2-carboxylate (1.57 g, 72%, colorless solid).

b) O-((4-Methoxyquinolin-2-yl)methyl)hydroxylamine was prepared from methyl 4-methoxyquinoline-2-carboxylate as described in EXAMPLE A6.

### Example A20

### O-((4-Fluorophenyl)(6-methoxypyridin-3-yl)methyl)hydroxylamine

was prepared from 6-methoxynicotinaldehyde as described in EXAMPLE A18.

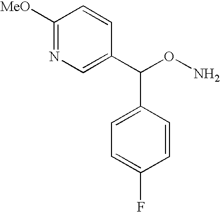

### Example A21

### 3-(1-(Aminooxy)-2-(tert-butyldimethylsilyloxy)ethyl)benzonitrile

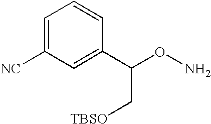

a) To a mixture of 3-acetylbenzonitrile (3.00 g, 20.4 mmol) and [bis(trifluoroacetoxy)iodo]benzene (17.6 g, 40.9 mmol) in acetonitrile/H₂O (5:1, 144 ml), trifluoroacetic acid (3.15 ml, 40.9 mmol) was added and stirred at 80° C. for 10 hours. The solvent was evaporated under reduced pressure and the residue was diluted with ethyl acetate (120 ml), washed with saturated aqueous NaHCO3 solution (30 ml), dried (MgSO₄ and K₂CO₃) and evaporated under reduced pressure. The residue was purified by column chromatography on silica (ethyl acetate/hexane, 45/55-65/35) to give 3-(2-hydroxyacetyl)benzonitrile (1.09 g, 33%, yellow solid).

b) To a solution of 3-(2-hydroxyacetyl)benzonitrile (1.08 g, 6.74 mmol) and imidazole (1.10 g, 16.2 mmol) in N,N-dimethylformamide (50 ml), tert-butylchlorodimethylsilyl chloride (2.10 g, 13.5 mmol) was added and stirred at 0° C. for 30 minutes. The solvent was evaporated under reduced pressure and the residue was diluted with ethyl acetate (120 ml), washed with saturated aqueous NH₄Cl solution (30 ml) and brine, dried (MgSO₄) and evaporated under reduced pressure. The residue was purified by column chromatography on silica (ethyl acetate/hexane, 0/100-20/80) to give 3-(2-(tert-butyldimethylsilyloxy)acetyl)benzonitrile (1.11 g, 60%, colorless oil).

c) To a solution of 3-(2-(tert-butyldimethylsilyloxy)acetyl)benzonitrile (1.10 g, 3.99 mmol) in methanol (20 ml), NaBH₄ (302 mg, 7.99 mmol) was added and stirred at 0° C. for 10 minutes. The solvent was evaporated under reduced pressure and the residue was diluted with ethyl acetate (100 ml), washed with saturated aqueous NaHCO₃ solution (30 ml) and brine, dried (MgSO₄) and evaporated under reduced pressure. The residue was purified by column chromatography on silica (ethyl acetate/hexane, 5/95-30/70) to give 3-(2-(tert-butyldimethylsilyloxy)-1-hydroxyethyl)benzonitrile (641 mg, 58%, colorless oil).

d) 3-(1-(Aminooxy)-2-(tert-butyldimethylsilyloxy)ethyl)benzonitrile was prepared from (4-methoxypyridin-2-yl)methanol as described in EXAMPLE A1.

### Example A22

### O-((5-Chlorobenzo[d]oxazol-2-yl)methyl)hydroxylamine

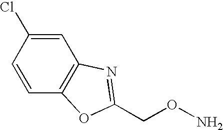

a) To a suspension of NaH (60%, 66 mg, 1.7 mmol) in N,N-dimethylfonnamide (5 ml), N-hydroxyphthalimide (269 mg, 1.65 mmol) and 5-chloro2-(chloromethyl)-1,3-benzoxazole (303 mg, 1.50 mmol) was added at 0° C. and stirred at 80° C. for 1 hour. The reaction was quenched with H₂O (3 ml) and the precipitated material was collected, washed with H₂O and dried under reduced pressure at 80° C. to give 2-((5-chlorobenzo[d]oxazol-2-yl)methoxy)isoindoline-1,3-dione.

b) A mixture of 2-((5-chlorobenzo[d]oxazol-2-yl)methoxy)isoindoline-1,3-dione (295 mg, 0.900 mmol) and hydrazine monohydrate (0.050 ml, 1 mmol) in ethanol (5 ml) was stirred at room temperature for 3 hours. After cooling to 0° C., precipitated materials were filtered off and the solvent was removed from the filtrate under reduced pressure to give the title compound (179 mg, 100%, yellow solid).

### Example A23

### O-((1H-Indol-2-yl)methyl)hydroxylamine

was prepared from (1H-indol-2-yl)methanol as described in EXAMPLE A1.

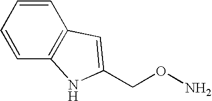

### Example A24

### O-((4-Fluorophenyl)(1-methyl-1H-imidazol-5-yl)methyl)hydroxylamine

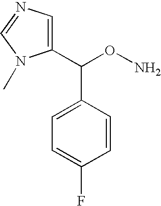

a) To a solution of 1-methyl-1H-imidazole-5-carboxaldehyde (500 mg, 4.45 mmol) in tetrahydrofuran (5 ml), 4-fluorophenylmagnesium bromide (1.0 M solution in tetrahydrofuran, 6.20 ml, 6.20 mmol) was added dropwise at 0° C. over 5 minutes and stirred at 0° C. for 30 minutes. The reaction was quenched with saturated aqueous NH₄Cl solution (30 ml), extracted with chloroform (50 ml×3), dried (MgSO₄) and evaporated under reduced pressure. The residue was purified by column chromatography on silica (ethyl acetate/methanol, 100/0-90/10) to give (4-fluorophenyl)(1-methyl-1H-imidazol-5-yl)methanol (443 mg, 48%, colorless solid).

b) To a solution of (4-fluorophenyl)(1-methyl-1H-imidazol-5-yl)methanol (270 mg, 1.31 mmol) in dichloromethane (10 ml), thionyl chloride (0.285 ml, 3.93 mmol) was added dropwise at 0° C. and stirred at room temperature for 1 hour. The solvent was evaporated under reduced pressure and the crude product of 5-(chloro(4-fluorophenyl)methyl)-1-methyl-1H-imidazole was used without further purification.

c) To a suspension of NaH (60%, 126 mg, 3.1 mmol) in N,N-dimethylformamide (8 ml), N-hydroxyphthalimide (256 mg, 1.57 mmol) was added at 0° C. and stirred at room temperature for 10 minutes. A solution of crude product of 5-(chloro(4-fluorophenyl)methyl)-1-methyl-1H-imidazole in N,N-dimethylformamide (7 ml) and KI (22 mg, 0.13 mmol) was added to the reaction mixture and stirred at 80° C. for 10 hours. The reaction was quenched with H₂O (30 ml), extracted with ethyl acetate (50 ml×2), washed with brine, dried (MgSO₄) and evaporated under reduced pressure. The residue was purified by column chromatography on silica (ethyl acetate/methanol, 100/0-90/10) to give 2-((4-fluorophenyl)(1-methyl-1H-imidazol-5-yl)methoxy)isoindoline-1,3-dione (411 mg, 89%, pale-yellow solid).

d) To a solution of 2-((4-fluorophenyl)(1-methyl-1H-imidazol-5-yl)methoxy)isoindoline-1,3-dione (200 mg, 0.569 mmol) in ethanol (2 ml), methylhydrazine (0.030 ml, 0.57 mmol) was added and stirred at room temperature for 2 hours. After cooling to 0° C., precipitated materials were filtered off and the solvent was removed from the filtrate under reduced pressure and the crude product of title compound was used without further purification.

### Example A25

### tert-Butyl 4-(aminooxy(4-fluorophenyl)methyl)piperidine-1-carboxylate

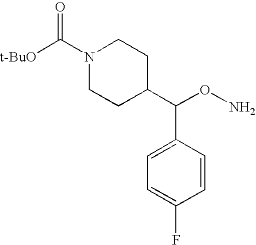

a) To a suspension of 4-(4-fluorobenzoyl)piperidine p-toluenesulfonate (2.00 g, 5.00 mmol) and triethylamine (0.840 ml, 6.00 mmol) in dichloromethane (55 ml), a solution of di-t-butyldicarbonate (1.20 g, 5.50 mmol) in dichloromethane (5 ml) was added and stirred at room temperature for 30 minutes. The reaction was quenched with saturated aqueous NaHCO₃ solution (50 ml), extracted with chloroform (60 ml), dried (MgSO₄) and evaporated under reduced pressure. The residue was purified by column chromatography on silica (ethyl acetate/hexane, 5/95-30/70) to give tert-butyl 4-(4-fluorobenzoyl)piperidine-1-carboxylate (1.56 g, 99%, colorless solid).

b) To a solution of tert-butyl 4-(4-fluorobenzoyl)piperidine-1-carboxylate (1.55 g, 4.93 mmol) in methanol (20 ml), NaBH₄ (302 mg, 7.99 mmol) was added and stirred at room temperature for 1 hour. The solvent was evaporated under reduced pressure and the residue was diluted with ethyl acetate (100 ml), washed with saturated aqueous NaHCO₃ solution (50 ml) and brine, dried (MgSO₄) and evaporated under reduced pressure to give tert-butyl 4-((4-fluorophenyl)(hydroxy)methyl)piperidine-1-carboxylate (1.62 g, 100%, colorless solid).

c) tert-butyl 4-(aminooxy(4-fluorophenyl)methyl)piperidine-1-carboxylate was prepared from tert-butyl 4-((4-fluorophenyl)(hydroxy)methyl)piperidine-1-carboxylate as described in EXAMPLE A1.

### Example A26

### O-((1H-Indol-3-ylmethyl)hydroxylamine

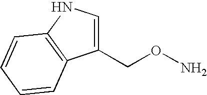

a) To a suspension of NaH (60%, 88 mg, 2.2 mmol) in N,N-dimethylformnamide (6 ml), N-hydroxyphthalimide (359 mg, 2.20 mmol) was added at 0° C. and stirred at room temperature for 5 minutes. N-Boc-3-bromoindole (620 mg, 2.00 mmol) was added to the reaction mixture at 0° C. and stirred at 80° C. for 1 hour. The reaction was quenched with H₂O, extracted with ethyl acetate, dried (MgSO₄) and evaporated under reduced pressure. The residue was purified by column chromatography on silica (ethyl acetate/hexane, 10/90-50/50) to give tert-butyl 3-((1,3-dioxoisoindolin-2-yloxy)methyl)-1H-indole-1-carboxylate (214 mg, 27%, pale-red solid).

b) A mixture of tert-butyl 3-((1,3-dioxoisoindolin-2-yloxy)methyl)-1H-indole-1-carboxylate (214 mg, 0.550 mmol) and hydrazine monohydrate (0.032 ml, 0.65 mmol) in ethanol (3 ml) was stirred at room temperature for 1 hour. After cooling to 0° C., precipitated materials were filtered off and the solvent was removed from the filtrate under reduced pressure. The residue was purified by column chromatography on silica (ethyl acetate/hexane, 20/80-50/50) to give tert-butyl 3-(aminooxymethyl)-1H-indole-1-carboxylate (86 mg, 60%, red oil).

c) To a solution of NaI (147 mg, 0.990 mmol) in acetonitrile (3 ml), chlorotrimethylsilane (0.130 ml, 0.990 mmol) was added and stirred at room temperature for 15 minutes. A solution of tert-butyl 3-(aminooxymethyl)-1H-indole-1-carboxylate (86 mg, 0.33 mmol) in acetonitrile (1 ml) was added to the reaction mixture and stirred at 60° C. for 1 hour. The reaction was quenched with H₂O and 10% aqueous Na₂S₂O₃ solution (3 ml), neutralized with ammonia solution, extracted with ethyl acetate, dried (MgSO₄) and evaporated under reduced pressure and the crude product of title compound was used without further purification.

### Example A27

### (S)-tert-Butyl 2-(aminooxymethyl)indoline-1-carboxylate

was prepared from (S)-tert-butyl 2-(hydroxymethyl)indoline-1-carboxylate as described in EXAMPLE A1.

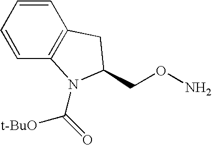

### Example A28

### O-((1H-Benzo[d]imidazol-6-yl)methyl)hydroxylamine

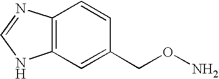

a) To a solution of 1H-benzimidazole-5-carboxylic acid (4.87 g, 30.0 mmol) in tetrahydrofuran (1 50 ml), LiAlH₄ (2.48 g, 60 mmol) was added at −78° C. and stirred at room temperature overnight. The reaction was quenched with methanol (5 ml) and H₂O (1 ml) at 0° C. and stirred at room temperature for 1.5 hours. The precipitated materials were filtered off and the filtrate was concentrated under reduced pressure. The residue was purified by column chromatography on silica (chloroform/methanol, 95/5-90/10) to give (1H-benzo[d]imidazol-6-yl)methanol (1.95 g, 44%, pale-yellow solid).

b) O-((1H-Benzo[d]imidazol-6-yl)methyl)hydroxylamine was prepared from (1H-benzo[d]imidazol-6-yl)methanol as described in EXAMPLE A1.

### Example A29

### O-(Benzo[d][1,3]dioxol-2-ylmethyl)hydroxylamine

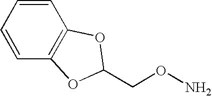

a) To a mixture of pyrocatechol (2.20 g,20.0 mmol)-and sodium methoxide (2.16 g, 40.0 mmol) in methanol (25 ml), dichloroacetic acid methyl ester (2.10 ml, 20.0 mmol) was added and refluxed for 17 hours. The reaction was quenched with HCl solution and the solvent was evaporated under reduced pressure. The residue was extracted with diethyl ether (50 ml×2), dried (Na₂SO₄) and evaporated under reduced pressure. The residue was purified by column chromatography on silica (ethyl acetate/hexane, 0/100-50/50) to give methyl benzo[d][1,3]dioxole-2-carboxylate (744 mg, 21%, colorless oil).

b) To a solution of methyl benzo[d][1,3]dioxole-2-carboxylate (735 mg, 4.08 mmol) in tetrahydrofuran (30 ml), a suspension of LiAlH₄ (176 mg, 4.49 mmol) in tetrahydrofuran (3 ml) was added at 0° C. and stirred at room temperature for 1 hour. The reaction was quenched with saturated aqueous NaHCO₃ solution (5 drops) and methanol (1 ml) at 0° C. and stirred at room temperature for 30 minutes. The precipitated materials were filtered off and the filtrate was concentrated under reduced pressure to give benzo[d][1,3]dioxol-2-ylmethanol (432 mg, 71%, colorless oil).

c) O-(Benzo[d][1,3]dioxol-2-ylmethyl)hydroxylamine was prepared from benzo[d][1,3]dioxol-2-ylmethanol as described in EXAMPLE A1.

### Example A30

### tert-Butyl 2-(aminooxymethyl)-5-fluoro-1H-benzo[d]imidazole-1-carboxylate

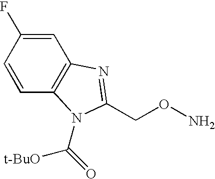

a) A mixture of 4-fluoro-1,2-phenylenediamine (1.26 g, 10.0 mmol) and chloroacetic acid (1.23 g, 13.0 mmol) in HCl (6 M, 7 ml) was stirred at 95° C. for 6 hours. The reaction mixture was neutralized with saturated aqueous NaHCO₃ solution (pH=9) and the precipitated material was collected, washed with H₂O and dried under reduced pressure at 65° C. to give 2-(chloromethyl)-5-fluoro-1H-benzo[d]imidazole (1.58 g, 86%, off-white solid).

b) To a solution of 2-(chloromethyl)-5-fluoro-1H-benzo[d]imidazole (921 mg, 5.00 mmol) in 1,4-dioxane (20 ml), di-t-butyldicarbonate (3.50 ml, 15.0 mmol) was added at room temperature and stirred at 90° C. for 6 hours. The reaction was quenched with H₂O, extracted with ethyl acetate, dried (MgSO₄) and evaporated under reduced pressure. The residue was purified by column chromatography on silica (ethyl acetate/hexane, 10/90-90/10) to give tert-butyl 2-(chloromethyl)-5-fluoro-1H-benzo[d]imidazole-1-carboxylate (982 mg, 70%, pale-yellow oil).

c) tert-Butyl 2-(aminooxymethyl)-5 -fluoro-1H-benzo[d]imidazole-1-carboxylate was prepared from tert-butyl 2-(chloromethyl)-5-fluoro-1H-benzo[d]imidazole-1-carboxylate as described in EXAMPLE A22.

### Example A31

### 3-(Aminooxy(pyridin-4-yl)methyl)benzonitrile

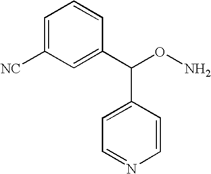

a) To a solution of bis(2-dimethylaminoethyl) ether (1.13 ml, 6.00 mmol) in tetrahydrofuran (25 ml), isopropylmagnesium chloride (2.0 M in tetrahydrofuran, 3.00 ml, 6.00 mmol) was added and stirred at room temperature for 30 minutes. To the reaction mixture, 3-iodobenzonitrile (1.14 g, 5.00 mmol) was added and stirred at room temperature for 10 minutes, and 4-pyridinecarboxaldehyde (0.480 ml, 5.00 mmol) was added and stirred at 0° C. for 1 hour. The reaction was quenched with saturated aqueous NH₄Cl solution, extracted with ethyl acetate (50 ml×3), dried (MgSO₄) and evaporated under reduced pressure. The residue was purified by column chromatography on silica (ethyl acetate/methanol, 100/0-80/20) to give 3-(hydroxy(pyridin-4-yl)methyl)benzonitrile (280 mg, 27%, colorless solid).

b) 3-(Aminooxy(pyridin-4-yl)methyl)benzonitrile was prepared from 3-(hydroxy(pyridin-4-yl)methyl)benzonitrile as described in EXAMPLE A24.

### Example A32

### 3-(Aminooxy(pyridin-3-yl)methyl)benzonitrile

was prepared from nicotinaldehyde as described in EXAMPLE A31.

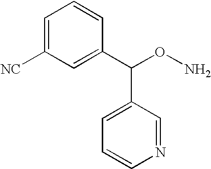

### Example A33

### 3-(Aminooxy(pyridin-2-yl)methyl)benzonitrile

was prepared from picolinaldehyde as described in EXAMPLE A31.

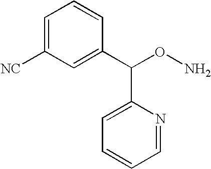

### Example A34

### 3-(1-(Aminooxy)-2-morpholinoethyl)benzonitrile

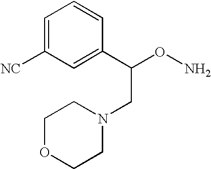

a) A solution of trimethylsulfonium iodide (4.49 g, 22.0 mmol) in acetonitrile (40 ml), KOH (2.64 g, 40.0 mmol) and H₂O (0.100 ml) was added at room temperature and stirred at 60° C. for 15 hours. The reaction was quenched with brine, extracted with diethyl ether (100 ml×2), washed with brine, dried (MgSO₄) and evaporated under reduced pressure. The residue was purified by column chromatography on silica (ethyl acetate/hexane, 25/75) to give 3-(oxiran-2-yl)benzonitrile (1.63 g, 55%, colorless oil).

b) A solution of 3-(oxiran-2-yl)benzonitrile (650 mg, 4.47 mmol) in morpholine (5 ml) was stirred at 80° C. for 20 hours. The solvent was evaporated under reduced pressure and the residue was purified by column chromatography on silica (chloroform/methanol, 97/3) to give 3-(1-hydroxy-2-morpholinoethyl)benzonitrile (983 mg, 95%, colorless oil).

c) 3-(1 -(Aminooxy)-2-morpholinoethyl)benzonitrile was prepared from 3-(1-hydroxy-2-morpholinoethyl)benzonitrile as described in EXAMPLE A1.

### Example A35

### 3-(1-(Aminooxy)-2-isopropoxyethyl)benzonitrile

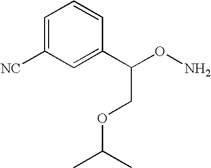

a) NaH (60%, 240 mg, 6.00 mmol) was added to isopropanol (1 ml) and stirred at room temperature for 15 minutes. A solution of 3-(oxiran-2-yl)benzonitrile (290 mg, 2.00 mmol) in isopropanol (4 ml) was added dropwise to the reaction mixture and refluxed for 8 hours. The reaction was quenched with H₂O (10 ml) at 0° C., extracted with diethyl ether (30 ml×2), washed with brine, dried (MgSO₄) and evaporated under reduced pressure. The residue was purified by column chromatography on silica (ethyl acetate/hexane, 30/70) to give 3-(1-hydroxy-2-isopropoxyethyl)benzonitrile (100 mg, 24%, colorless oil).

b) 3-(1-(Aminooxy)-2-isopropoxyethyl)benzonitrile was prepared from 3-(1-hydroxy-2-isopropoxyethyl)benzonitrile as described in EXAMPLE A1.

### Example A36

### 3-( 1 -(Aminooxy)-2-(4-fluorophenoxy)ethyl)benzonitrile

was prepared from 4-fluorophenol as described in EXAMPLE A35.

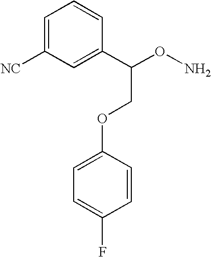

### EXAMPLE A37

### tert-Butyl 2-(aminooxymethyl)-3H-imidazo[4,5-b]pyridine-3-carboxylate

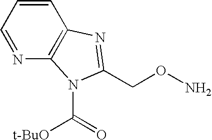

a) After a mixture of 3,4-diaminopyridine (546 mg, 5.00 mmol) in polyphosphoric acid (18 g) was heated at at 120° C. for 20 minutes, chloroacetonitrile (0.410 ml, 6.50 mmol) was added to the reaction mixture and heated under microwave irradiation at 200° C. for 30 minutes. The reaction was quenched with H₂O (10 ml) at 0° C., poured into saturated aqueous NaHCO₃ solution, extracted with ethyl acetate/methanol, dried (MgSO₄) and evaporated under reduced pressure. The residual solid was triturated with ethyl acetate/hexane (40:60) to give 2-(chloromethyl)-3H-imidazo[4,5-b]pyridine (510 mg, 61%, yellow solid).

b) tert-Butyl 2-(aminooxymethyl)-3H-imidazo[4,5-b]pyridine-3-carboxylate was prepared from 2-(chloromethyl)-3H-imidazo[4,5-b]pyridine as described in EXAMPLE A30.

### Example A38

### O-(Cyclopropyl(4-fluorophenyl)methyl)hydroxylamine

was prepared from 4-fluorobenzaldehyde as described in EXAMPLE A18.

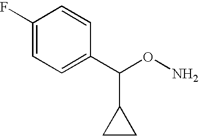

### Example A39

### O-(Cyclopropyl(3-(trifluoromethyl)phenyl)methyl)hydroxylamine

was prepared from 3-trifluoromethylbenzaldehyde as described in EXAMPLE A18.

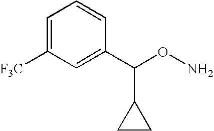

### Example A40

### tert-Butyl 2-(aminooxymethyl)-6-chloro-3H-imidazo[4,5-b]pyridine-3-carboxylate

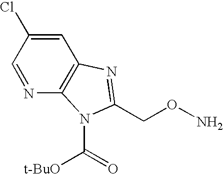

a) To a mixture of 2-amino-5-chloropyridine (2.57 g, 20.0 mmol) and sulfuric acid (6.30 ml), huming HNO₃ (0.860 ml, 21.1 mmol) was added dropwise over 15 minutes at 55° C. and stirred at 55° C. for 1 hour. After cooling, the reaction mixture was poured into ice-water (60 g) and neutralized with aqueous NaOH solution (pH=11). The precipitated material was collected, washed with H₂O and dried under reduced pressure at 50° C. to give 5-chloro-3-nitropyridin-2-amine (2.31 g, 67%, yellow solid).

b) To a suspension of 5-chloro-3-nitropyridin-2-amine (1.04 g, 6.00 mmol) in ethanol (6 ml), SnCl₂.2H₂O (6.77 g, 30.0 mmol) was added portionwise at room temperature and stirred at 90° C. for 1 hour. After cooling, the reaction mixture was basified with aqueous NaOH solution (pH=14), extracted with ethyl acetate, dried (MgSO₄) and evaporated under reduced pressure to give 5-chloropyridine-2,3-diamine (604 mg, 70%, tan solid).

c) tert-Butyl 2-(aminooxymethyl)-6-chloro-3H-imidazo[4,5-b]pyridine-3-carboxylate was prepared from 5-chloropyridine-2,3-diamine as described in EXAMPLE A37.

### Example A41

### O-((3-Phenylpyridin-2-yl)methyl)hydroxylamine

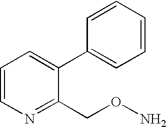

a) A mixture of 3-hydroxypicolic acid (1.39 g, 10.0 mmol) and HCl (2 M in methanol, 60 ml) was refluxed for 6 hours. The solvent was removed under reduced pressure and the residue was neutralized with saturated aqueous NaHCO₃ solution, extracted with ethyl acetate, dried (MgSO₄) and evaporated under reduced pressure to give methyl 3-hydroxypicolinate (687 mg, 45%, colorless solid).

b) To a solution of 3-hydroxypicolinate (687 mg, 4.49 mmol) and triethylamine (0.690 ml, 4.93 mmol) in dichloromethane (10 ml), trifluoromethanesulfonic anhydride (0.830 ml, 4.93 mmol) was added dropwise at 0° C. and stirred at room temperature for 1 hour. The reaction was quenched with H₂O, extracted with chloroform, dried (MgSO₄) and evaporated under reduced pressure. The residue was purified by column chromatography on silica (ethyl acetate/hexane, 20/80-50/50) to give methyl 3-(trifluoromethylsulfonyloxy)picolinate (1.22 g, 95%, pale-yellow oil).

c) A mixture of methyl 3-(trifluoromethylsulfonyloxy)picolinate (610 mg, 2.14 mmol), phenylboronic acid (274 mg, 2.25 mmol), tetrakis(triphenylphosphine)palladium (74 mg, 0.064 mmol) and K₂CO₃ (591 mg, 4.28 mmol) in toluene (10 ml) was stirred at 90° C. for 1 hour. After cooling, the reaction was quenched with H₂O, extracted with ethyl acetate, dried (MgSO₄) and evaporated under reduced pressure. The residue was purified by column chromatography on silica (ethyl acetate/hexane, 30/70-50/50) to give methyl 3-phenylpicolinate (467 mg, 100%, yellow oil).

d) To a solution of methyl 3-phenylpicolinate (467 mg, 2.19 mmol).in tetrahydrofuran (5 ml), LiBH₄ (72 mg, 3.3 mmol) was added at 0° C. and stirred at room temperature for 1.5 hours. The reaction was quenched with H₂O and extracted with ethyl acetate, dried (MgSO₄) and evaporated under reduced pressure. The residue was purified by column chromatography on silica (ethyl acetate/hexane, 30/70-50/50) to give (3-phenylpyridin-2-yl)methanol (186 mg, 46%, yellow oil).

e) O-((3-Phenylpyridin-2-yl)methyl)hydroxylamine was prepared from (3-phenylpyridin-2-yl)methanol as described in EXAMPLE A1.

### Example A42

### O-((2-Chloropyridin-3-yl)methyl)hydroxylamine

was prepared from (2-chloropyridin-3-yl)methanol as described in EXAMPLE A1.

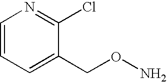

### Example A43

### O-((3-Chloropyridin-4-yl)methyl)hydroxylamine

was prepared from 3-chloro-4-(chloromethyl)pyridine as described in EXAMPLE A22.

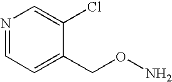

### Example A44

### O-((6-Chloropyridin-2-yl)methyl)hydroxylamine

was prepared from 2-chloro-6-(chloromethyl)pyridine as described in EXAMPLE A22.

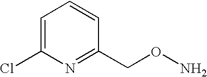

### Example A45

### O-((4-Chloropyridin-3-yl)methyl)hydroxylamine

was prepared from 4-chloro-3-(chloromethyl)pyridine as described in EXAMPLE A22.

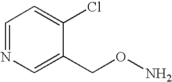

### Example A46

### O-((2-Chloropyridin-4-yl)methyl)hydroxylamine

was prepared from 2-chloro-4-(chloromethyl)pyridine as described in EXAMPLE A22.

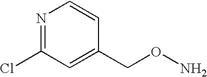

### Example A47

### O-((3-Chloropyridin-2-yl)methyl)hydroxylamine

was prepared from 3-chloropyridine as described in EXAMPLE A15.

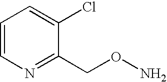

### Example A48

### 5-(Aminooxymethyl)nicotinonitrile

was prepared from 5-(chloromethyl)nicotinonitrile as described in EXAMPLE A22.

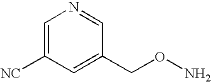

### Example 1

### 1-((S)-4-Methyl-2-methylaminopentanoyl)piperidin-4-one O-(3-trifluoromethylbenzyl)oxime hydrochloride

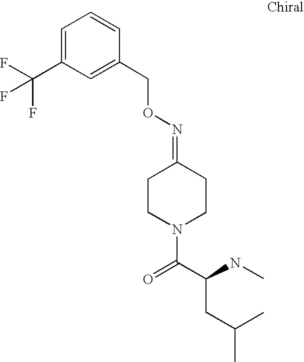

a) 4-Piperidone monohydrate hydrochloride (1.10 g, 7.00 mmol) and N-Boc-N-methylleucine (1.89 g, 7.70 mmol) were suspended in N,N-dimethylformamide (70 ml). 1-Hydroxybenzotriazole (946 mg, 7.00 mmol), N-ethyldimethylaminopropylcarbodiimide hydrochloride (1.61 g, 8.40 mmol) and triethylamine (1.17 ml, 8.40 mmol) were added to the suspension, and the mixture was stirred at 60° C. for 8 hours. The solvent was evaporated under reduced pressure. The residue was diluted with ethyl acetate (50 ml), poured into saturated aqueous NaHCO3 solution (50 ml) and extracted with ethyl acetate (100 ml×2). The organic layer was washed with brine, dried (MgSO₄) and evaporated under reduced pressure. The residue was purified by column chromatography on silica (ethyl acetate/hexane, 40/60-60/40) to give [(S)-1-(4,4-dihydroxypiperidine-1-carbonyl)-3-methylbutyl]methylcarbamic acid tert-butyl ester (2.28 g, 94%, pale yellow solid).

b) [(S)-1-(4,4-Dihydroxypiperidine-1-carbonyl)-3-methylbutyl]methylcarbamic acid tert-butyl ester (172 mg, 0.50 mmol), O-(3-trifluoromethylbenzyl)hydroxylamine hydrochloride (114 mg, 0.50 mmol) and sodium acetate (41 mg, 0.50 mmol) were suspended in ethanol (5 ml). The mixture was stirred at room temperature for 1 hour. The solvent was evaporated under reduced pressure, the residue was diluted with ethyl acetate (20 ml), washed with H₂O (5 ml), dried (MgSO₄) and evaporated under reduced pressure. The residue was purified by column chromatography on silica (ethyl acetate/hexane, 20/80-50/50) to give [(S)-1-(4-benzyloxyiminopiperidine-1-carbonyl)-3-methylbutyl]methylcarbamic acid tert-butyl ester (250 mg, 98%, colorless oil).

c) [(S)-1-(4-Benzyloxyiminopiperidine-1-carbonyl)-3-methylbutyl]methylcarbamic acid tert-butyl ester (250 mg, 0.50 mmol) was dissolve in ethyl acetate (2.5 ml) and cooled to 0° C. The mixture was added 4N—HCl in ethyl acetate (2.5 ml) and stirred at room temperature for 8 hours. The solvent was removed under reduced pressure. The residue was triturated with ether (10 ml) to give the title compound (170 mg, 80%).

### Example 2

### 1-((S)-4-Methyl-2-methylaminopentanoyl)piperidin-4-one O-benzyloxime hydrochloride

was prepared as described in EXAMPLE 1

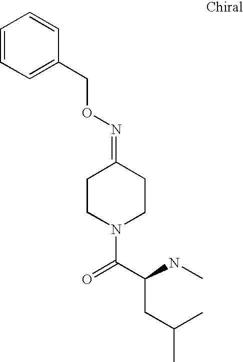

### Example 3

### 1-((S)-4-Methyl-2-methylaminopentanoyl)piperidin-4-one O-(4-trifluoromethylbenzyl)oxime hydrochloride

was prepared as described in EXAMPLE 1

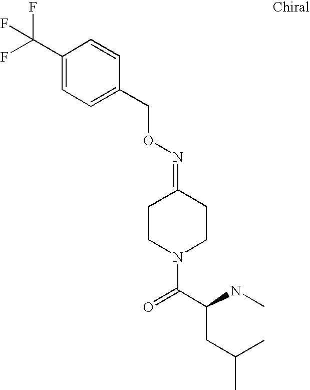

### Example 4

### 1-((S)-4-Methyl-2-methylaminopentanoyl)piperidin-4-one O-(4-chlorobenzyl)oxime hydrochloride

was prepared as described in EXAMPLE 1.

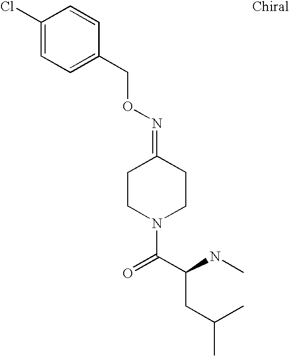

¹H—NMR (DMSO-d₆) δ: 9.02 (1H, brs), 7.41 (2H, d), 7.39 (2H, d), 5.01 (2H, s), 4.52-4.40 (1H, m), 3.92-3.68 (2H, m), 3.58-3.38 (2H, m), 2.80-2.36 (7H, m), 1.80-1.52 (3H, m), 0.98-0.82 (6H, m).

### Example 5

### 1-((S)-4-Methyl-2-methylaminopentanoyl)piperidin-4-one O-pyridin-4-ylmethyloxime oxalate

was prepared as described in EXAMPLE 1. The free base was dissolved in ether and oxalic acid (2.0 eq) was added to the solution. The resulting precipitate was collected and dried in vacuo to give the title compound.

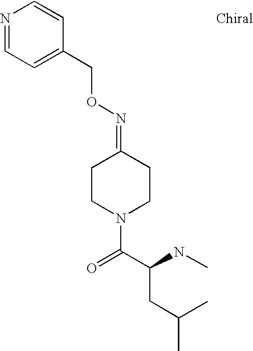

### Example 6

### 1-((S)-4-Methyl-2-methylaminopentanoyl)piperidin-4-one O-pyridin-2-ylmethyl-oxime oxalate

was prepared as described in EXAMPLE 1. Starting material was O-pyridin-2-ylmethylhydroxylamine dihydrochloride (see example A1). The free base was dissolved in ether and oxalic acid (2.0 eq) was added to the solution. The resulting precipitate was filtrated and dried in vacuo to give the title compound.

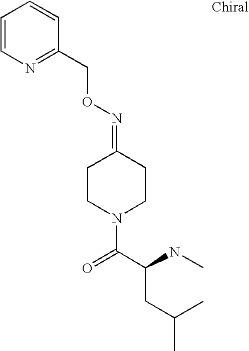

### Example 7

### 1-((S)-4-Methyl-2-methylaminopentanoyl)piperidin-4-one O-(2-chloropyridin-4-ylmethyl)oxime oxalate

was prepared as described in EXAMPLE 1. Starting material was O-(2-chloropyridin-4-ylmethyl)hydroxylamine (see example A2).

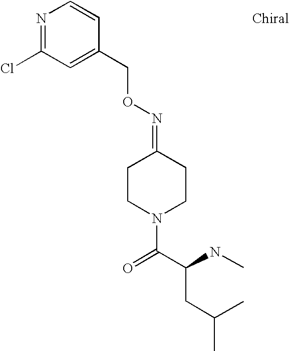

### Example 8

### 1-((S)-2-Amino-4-methylpentanoyl)piperidin-4-one O-(3-trifluoromethylbenzyl)oxime hydrochloride

was prepared as described in EXAMPLE 1. BOC group was deprotected by using trifluoroacetic acid.

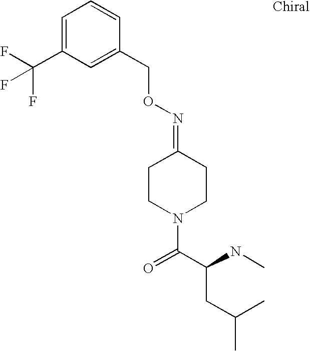

¹H—NMR (DMSO-d₆) δ: 8.32 (1H, s), 7.71-7.57 (4H, m), 5.18 (2H, s), 4.41-4.38 (1H, m), 3.78-3.74 (2H, m), 3.50-3.23 (2H, m), 2.81-2.31 (4H, m), 1.85-1.62 (3H, m), 0.97-0.93 (6H, m).

### Example 9

### 1-((S)-4-Methyl-2-methylaminopentanoyl)piperidin-4-one O-(3-chlorobenzyl)oxime hydrochloride

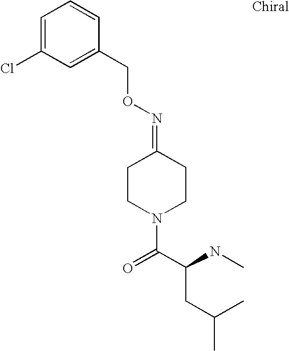

a) [(S)-1-(4,4-Dihydroxypiperidine-1-carbonyl)-3-methylbutyl]methylcarbamic acid tert-butyl ester (517 mg, 1.50 mmol), hydroxylamine hydrochloride (125 mg, 1.80 mmol) and sodium acetate (150 mg, 1.80 mmol) were suspended in ethanol (15 ml). The mixture was stirred at room temperature for 2 hours. The solvent was evaporated under reduced pressure. The residue was diluted with ethyl acetate (50 ml), washed with H₂O (20 ml), dried (MgSO₄) and evaporated under reduced pressure to give [(S)-1-(4-hydroxyiminopiperidine-1-carbonyl)-3-methylbutyl]methylcarbamic acid tert-butyl ester (512 mg, 100%, colorless solid).

b) [(S)-1-(4-Hydroxyiminopiperidine-1-carbonyl)-3-methylbutyl]methylcarbamic acid tert-butyl ester (125 mg, 0.365 mmol) and 3-chlorobenzyl bromide (83 mg, 0.402 mmol) was dissolved in N,N-dimethylformamide (5 ml). The solution was cooled to 0° C. and NaH (31 mg, 2.1 mmol) was added to the mixture under nitrogen atmosphere. The mixture was stirred at 0° C. for 1 hour and at room temperature for 30 minutes. The reaction was quenched with H₂O (15 ml) and extracted with ethyl acetate (30 ml×2). The organic layer was washed with brine, dried (MgSO₄) and evaporated under reduced pressure. The residue was purified by column chromatography on silica (ethyl acetate/hexane, 20/80-40/60) to give {(S)-1-[4-(3-chlorobenzyloxyimino)piperidine-1-carbonyl]-3-methylbutyl}methylcarbamic acid tert-butyl ester (139 mg, 82%, colorless oil).

c) {(S)-1-[4-(3-Chlorobenzyloxyimino)piperidine-1-carbonyl]-3-methylbutyl}methylcarbamic acid tert-butyl ester (135 mg, 0.290 mmol) was dissolve in ethyl acetate (1 ml) and cooled to 0° C. The mixture was added 4N—HCl in ethyl acetate (2 ml) and stirred at room temperature for 6 hours. The solvent was removed under reduced pressure and the resulting precipitate was recrystallized from ethyl acetate/methanol to give the title compound (74 mg, 63%).

### Example 10

### 3-[1-((S)-4-Methyl-2-methylaminopentanoyl)piperidin-4-ylideneaminooxymethyl]benzonitrile hydrochloride

was prepared as described in EXAMPLE 9

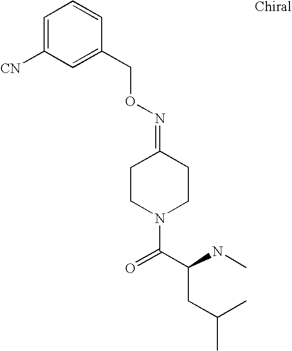

### Example 11

### 1-((S)-4-Methyl-2-methylaminopentanoyl)piperidin-4-one O-(2-trifluoromethylbenzyl)oxime hydrochloride

was prepared as described in EXAMPLE 9

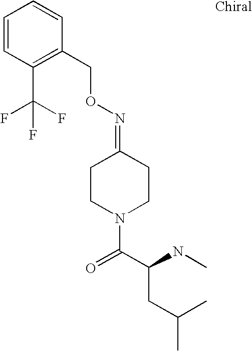

### Example 12

### 1-[4,4-Bis-(4-fluorophenyl)butyl]piperidin-4-one O-(3-trifluoromethylbenzyl)oxime oxalate

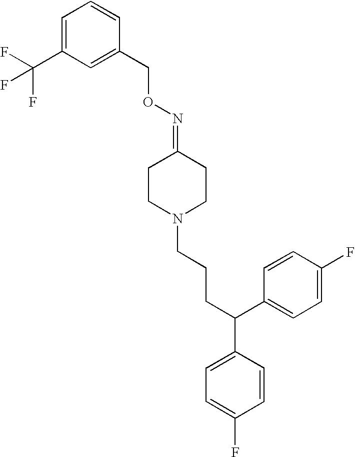

a) 1,4-Dioxa-8-azaspiro[4.5]decane (1.43 g, 10.0 mmol), and 1,1′-(4-chlorobutylidene)-bis(4-fluorobenzene) (3.43 g, 11.0 mmol) was dissolved in acetonitrile (100 ml). KI (332 mg, 2.0 mmol) and K₂CO₃ (3.32 g, 24.0 mmol), was added to the solution, and the mixture was refluxed for 2 days. The solvent was removed under reduced pressure. H₂O (50 ml), was added to the residue, extracted with chloroform (80 ml×2), dried (MgSO₄) and evaporated under reduced pressure. The residue was purified by column chromatography on silica (ethyl acetate/methanol, 100/0-90/10) to give 8-[4,4-bis-(4-fluorophenyl)butyl]-1,4-dioxa-8-azaspiro[4.5]decane (3.45 g, 89%, colorless oil).

b) To a solution of 8-[4,4-Bis-(4-fluorophenyl)butyl]-1,4-dioxa-8-azaspiro[4.5]decane (3.09 g, 7.98 mmol) in tetrahydrofuran (50 ml), 2N—HClaq. (16 ml) was added and refluxed for 24 hours. The mixture was neutralized with 2N—NaOHaq. at 0° C. and extracted with ethyl acetate (50 ml×2). The organic layer was washed with brine, dried (MgSO₄) and evaporated under reduced pressure. The residue was purified by column chromatography on silica (ethyl acetate/methanol, 100/0-90/10) to give 1-[4,4-bis-(4-fluorophenyl)butyl]piperidine-4,4-diol (2.41 g, 84%, pale yellow oil).

c) 1-[4,4-Bis-(4-fluorophenyl)butyl]piperidine-4,4-diol (289 mg, 0.80 mmol), O-(3-trifluoromethylbenzyl)hydroxylamine hydrochloride (182 mg, 0.80 mmol) and sodium acetate (66 mg, 0.80 mmol) were suspended in ethanol (5 ml). The mixture was stirred at room temperature for 1 hour. The solvent was evaporated under reduced pressure. The residue was diluted with ethyl acetate (20 ml), washed with H₂O (5 ml), dried (MgSO₄) and evaporated under reduced pressure. The residue was purified by column chromatography on silica (ethyl acetate/hexane, 30/70-60/40) to give 1-[4,4-bis-(4-fluorophenyl)butyl]piperidin-4-one O-(3-trifluoromethylbenzyl)oxime (398 mg, 96%, colorless oil). To a solution of this free base in ethanol (2 ml), a solution of oxalic acid (79 mg, 0.85 mmol) in ethanol (1 ml) was added. The resulting precipitate was collected and dried to give the title compound.

¹H—NMR (DMSO-d₆) δ: 7.73-7.57 (4H, m), 7.35-7.29 (4H, m), 7.15-7.07 (4H, m), 5.12 (2H, s), 4.09-4.00 (1H, m), 2.99-2.88 (6H, m), 2.71-2.67 (2H, m), 2.41-2.38 (2H, m), 2.05-1.91 (2H, m), 1.48-1.44 (2H, m).

### Example 13

### 1-[4,4-Bis-(4-fluorophenyl)butyl]piperidin-4-one O-(4-chlorobenzyl)oxime oxalate

was prepared as described in EXAMPLE 12

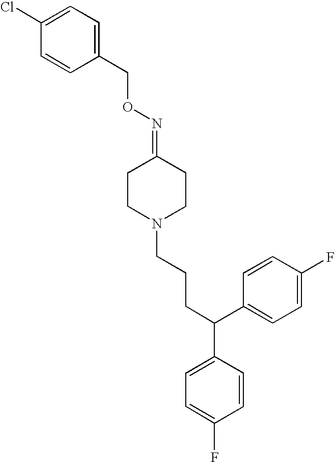

### Example 14

### 1-[4,4-Bis-(4-fluorophenyl)butyl]piperidin-4-one O-phenyloxime oxalate

was prepared as described in EXAMPLE 12

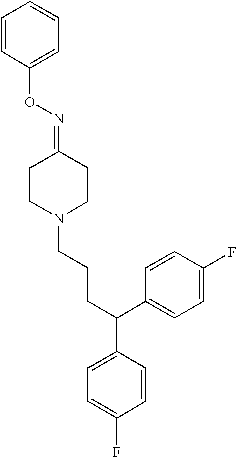

### Example 15

### 1-[4,4-Bis-(4-fluorophenyl)butyl]piperidin-4-one O-phenethyloxime oxalate

was prepared as described in EXAMPLE 12

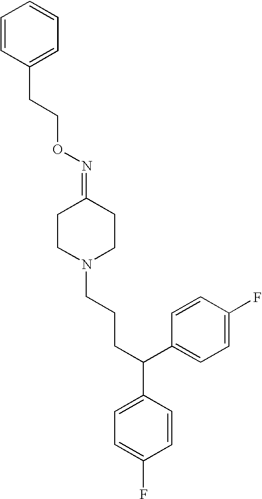

### Example 16

### 1-[4,4-Bis-(4-fluorophenyl)butyl]piperidin-4-one O-pyridin-3-ylmethyloxime dioxalate

was prepared as described in EXAMPLE 12

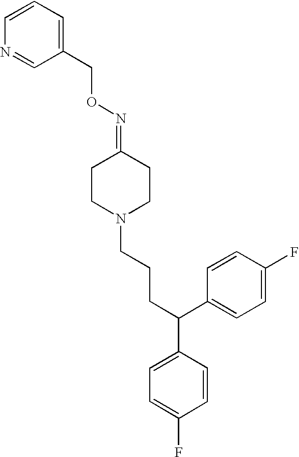

¹H—NMR (DMSO-d₆) δ: 8.56 (1H, d), 8.52 (1H, dd), 7.76 (1H, dt), 7.39 (1H, dd), 7.33 (4H, dd), 7.11 (4H, t), 5.08 (2H, s), 4.03-3.98 (1H, m), 3.34-3.10 (6H, m), 2.76-2.72 (2H, m), 2.05-1.91 (2H, m), 1.53-1.50 (2H, m).

### Example 17

### 1-[4,4-Bis-(4-fluorophenal)butyl]piperidin-4-one O-pyridin-4-ylmethyloxime dioxalate

was prepared as described in EXAMPLE 12

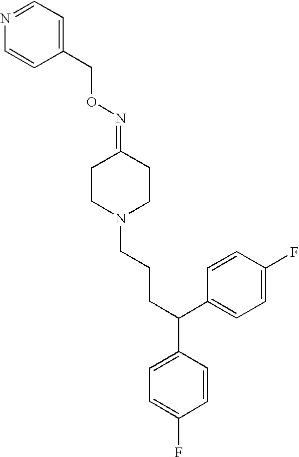

### Example 18

### 1-[4,4-Bis-(4-fluorophenyl)butyryl]piperidin-4-one O-pyridin-3-ylmethyloxime oxalate

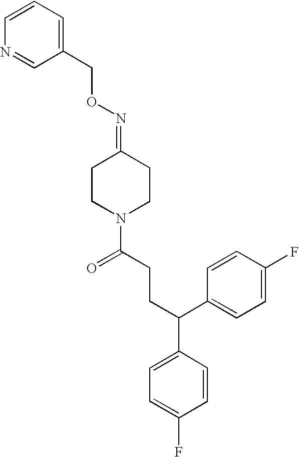

a) 4-Piperidone monohydrate hydrochloride (313 mg, 2.00 mmol) and 4,4-bis-(4-fluorophenyl)butyric acid (608 mg, 2.20 mmol) were suspended in N,N-dimethylformamide (20 ml). 1-Hydroxybenzotriazole (270 mg, 2.00 mmol), N-ethyldimethylaminopropylcarbodiimide hydrochloride (460 mg, 2.40 mmol) and triethylamine (0.225 ml, 2.40 mmol) were added to the suspension and the mixture was stirred at 50° C. for 8 hours. The solvent was evaporated under reduced pressure. The residue was diluted with chloroform (10 ml), poured into saturated aqueous NaHCO₃ solution (30 ml), extracted with chloroform (100 ml×2), dried (MgSO₄) and evaporated under reduced pressure. The residue was purified by column chromatography on silica (ethyl acetate/hexane, 50/50-70/30) to give 1-(4,4-dihydroxypiperidin-1-yl)-4,4-bis-(4-fluorophenyl)butan-1-one (668 mg, 89%, pale yellow oil).

b) 1-(4,4-Dihydroxypiperidin-1-yl)-4,4-bis-(4-fluorophenyl)butan-1-one (300 mg, 0.80 mmol), O-pyridin-3-ylmethylhydroxylamine dihydrochloride (158 mg, 0.80 mmol) and sodium acetate (131 mg, 1.60 mmol) were suspended in ethanol (8 ml). The mixture was stirred at room temperature for 2 hours. The solvent was evaporated under reduced pressure. The residue was diluted with ethyl acetate (30 ml), washed with H₂O (10 ml), dried (MgSO₄) and evaporated under reduced pressure. The residue was purified by column chromatography on silica (ethyl acetate/methanol, 100/0-90/10) to give 1-[4,4-bis-(4-fluorophenyl)butyryl]piperidin-4-one O-pyridin-3-ylmethyloxime (307 mg, 83%, pale yellow oil). To a solution of this free base in ether (5 ml), oxalic acid (62 mg, 0.664 mmol) in ether (2 ml) was added. The resulting precipitate was collected and dried to give the title compound.

¹H—NMR (DMSO-d₆) δ: 8.60-8.46 (2H, m), 7.76 (1H, d), 7.40 (1H, dd), 7.38-7.23 (4H, dd), 7.08 (4H, t), 5.02 (2H, s), 4.04-3.98 (1H, m), 3.58-3.28 (4H, m), 2.51-2.41 (2H, m), 2.31-2.08 (6H, m).

### Example 19

### 1-[4,4-Bis-(4-fluorophenyl)butyryl]piperidin-4-one O-pyridin-4-ylmethyloxime oxalate

was prepared as described in EXAMPLE 18

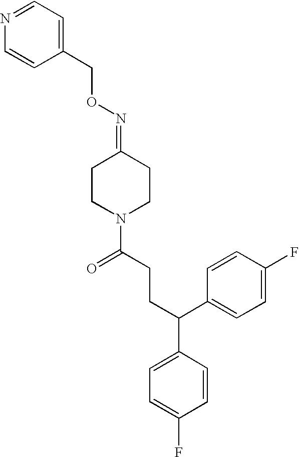

¹H—NMR (DMSO-d₆) δ: 8.55-8.50 (2H, m), 7.34-7.32 (6H, m), 7.12-7.09 (4H, m), 5.10 (2H, s), 4.00 (1H, t), 3.16-3.08 (6H, m), 2.83-2.80 (2H, m), 2.51-2.49 (2H, m), 2.03-2.01 (2H, m), 1.55-1.52 (2H, m)

### Example 20

### 1-Benzoylpiperidin-4-one O-(3-trifluoromethylbenzyl)oxime

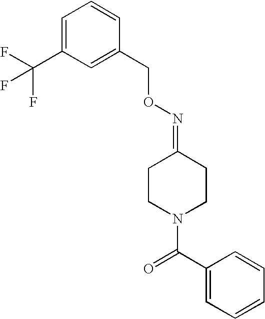

Piperidin-4-one O-(3-trifluoromethylbenzyl)oxime (218 mg, 0.80 mmol) and benzoic acid (107 mg, 0.88 mmol) were dissolved in dichloromethane (4 ml). 1-Hydroxybenzotriazole (108 mg, 0.80 mmol) and N-ethyldimethylaminopropylcarbodiimide hydrochloride (184 mg, 0.96 mmol) were added to the mixture and stirred at room temperature for 1 hour. Saturated aqueous NaHCO₃ solution (10 ml) was added, extracted with chloroform (30 ml×2), dried (MgSO₄) and evaporated under reduced pressure. The residue was purified by column chromatography on silica (ethyl acetate/hexane, 20/80-40/60) to give the title compound (193 mg, 64%).

### Example 21

### 1-Phenyl-2-[4-(3-trifluoromethylbenzyloxyimino)piperidin-1-yl]ethane-1,2-dione

was prepared as described in EXAMPLE 20

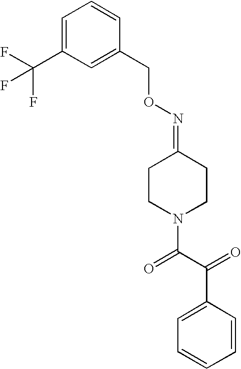

### Example 22

### 2-Oxo-N-phenyl-2-[4-(3-trifluoromethylbenzyloxyimino)piperidin-1-yl]acetamide

was prepared as described in EXAMPLE 20

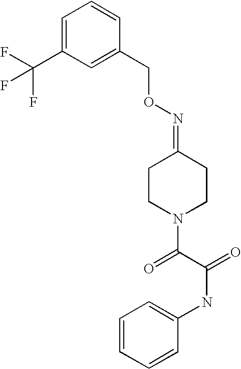

### Example 23

### 4-(3-Trifluoromethylbenzyloxyimino)piperidine-1-carboxylic acid phenylamide

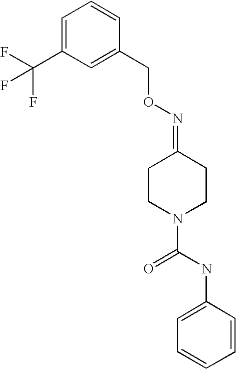

To a solution of piperidin-4-one O-(3-trifluoromethylbenzyl)oxime (218 mg, 0.80 mmol) in acetonitrile (2 ml), a solution of phenylisocyanate (114 mg, 0.96 mmol) in acetonitrile (2 ml) was added. The mixture and stirred at room temperature for 30 minutes. Methanol (10 ml) was added and evaporated under reduced pressure. The residue was purified by column chromatography on silica (ethyl acetate/hexane, 10/80-40/60) to give the title compound (178 mg, 57%).

### Example 24

### 1-((S)-3-Amino-5-methylhexanoyl)piperidin-4-one O-(3-trifluoromethylbenzyl)oxime oxalate

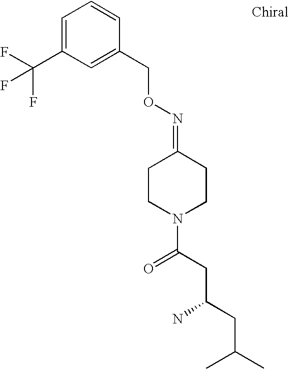

a) 1-Boc-4-piperidone (2.03 g, 10.0 mmol), O-(3-trifluoromethylbenzyl)hydroxylamine hydrochloride (2.28 g, 10.0 mmol) and sodium acetate (820 mg, 10.0 mmol) were suspended in ethanol (100 ml). The mixture was stirred at room temperature for 3 hours. The solvent was evaporated under reduced pressure. The residue was diluted with ethyl acetate (100 ml), washed with H₂O (30 ml), dried (MgSO₄) and evaporated under reduced pressure. The residue was purified by column chromatography on silica (ethyl acetate/hexane, 5/95-25/75) to give 4-(3-trifluoromethylbenzyloxyimino)piperidine-1-carboxylic acid tert-butyl ester (3.15 g, 85%, colorless oil).

b) To a solution of 4-(3-trifluoromethylbenzyloxyimino)piperidine-1-carboxylic acid tert-butyl ester (3.14 g, 8.34 mmol) in ethyl acetate (30 ml), 4N—HCl in ethyl acetate (30 ml) was added at 0° C. and stirred at room temperature for 2 hours. The solvent was removed under reduced pressure to give piperidin-4-one O-(3-trifluoromethylbenzyl)oxime hydrochloride (2.57 g, 100%, pale yellow solid).

c) To a solution of piperidin-4-one O-(3-trifluoromethylbenzyl)oxime hydrochloride (247 mg, 0.80 mmol) and N-Boc-L-β-homoleucine (216 mg, 0.88 mmol) in dichloromethane (4 ml), 1-hydroxybenzotriazole (108 mg, 0.80 mmol), N-ethyldimethylaminopropylcarbodiimide hydrochloride (184 mg, 0.96 mmol) and triethylamine (0.135 ml, 0.96 mmol) was added and stirred at room temperature for 2 hours. Saturated aqueous NaHCO₃ solution (10 ml) was added, extracted with chloroform (20 ml×2), dried (MgSO₄) and evaporated under reduced pressure. The residue was purified by column chromatography on silica (ethyl acetate/hexane, 35/65-60/40) to give ((S)-3-methyl-1-{2-oxo-2-[4-(3-trifluoromethylbenzyloxyimino)piperidin-1-yl]ethyl}butyl)carbamic acid tert-butyl ester (340 mg, 85%, colorless oil).

d) To a solution of ((S)-3-Methyl-1-{2-oxo-2-[4-(3-trifluoromethylbenzyloxyimino)piperidin-1-yl]ethyl}butyl)carbamic acid tert-butyl ester (340 mg, 0.646 mmol) in chloroform (8 ml), trifluoroacetic acid (4 ml) was added at 0° C. and stirred at room temperature for 2 hours. The solvent was removed under reduced pressure. Saturated aqueous NaHCO₃ solution (10 ml) was added and extracted with ethyl acetate (30 ml×2). The organic layer was washed with brine, dried (MgSO₄) and evaporated under reduced pressure. The residue was purified by column chromatography on silica (chloroform/methanol, 95/5-85/15) to give 1-((S)-3-amino-5-methylhexanoyl)piperidin-4-one O-(3-trifluoromethylbenzyl)oxime (243 mg, 90%, pale yellow oil). To a solution of this free base in ethanol (3 ml), oxalic acid (55 mg, 0.608 mmol) in ethanol (1 ml) was added. The solvent was removed under reduced pressure to give the title compound.

¹H—NMR (DMSO-d₆) δ: 7.66-7.57 (4H, m), 5.13 (2H, s), 3.57-3.45 (3H, m), 2.78-2.28 (6H, m), 1.76-1.67 (1H, m), 1.40 (2H, dt), 0.87 (6H, t).

### Example 25

### 1-((S)-2-Amino-3-phenylpropionyl)piperidin-4-one O-(3-trifluoromethylbenzyloxime oxalate

was prepared as described in EXAMPLE 24

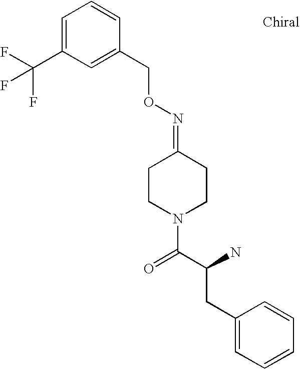

### Example 26

### 1-((S)-2-Amino-4-methylpentanoyl)piperidin-4-one O-benzyloxime hydrochloride

was prepared as described in EXAMPLE 24. Boc group was deprotected by using 4N—HCl in ethyl acetate.

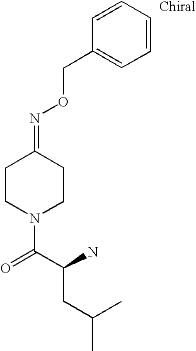

### Example 27

### 1-((S)-3-Amino-5-methylhexanoyl)piperidin-4-one O-benzyloxime hydrochloride

was prepared as described in EXAMPLE 24. Boc group was deprotected by using 4N—HCl in ethyl acetate.

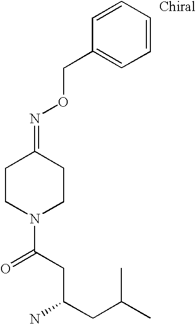

### Example 28

### 1-((S)-2-Amino-3-phenylpropionyl)piperidin-4-one O-benzyloxime hydrochloride

was prepared as described in EXAMPLE 24. Boc group was deprotected by using 4N—HCl in ethyl acetate.

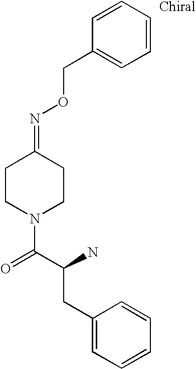

### Example 29

### 1-[4,4-Bis-(4-fluorophenyl)butyryl]piperidin-4-one O-(3-trifluoromethylbenzyl)oxime

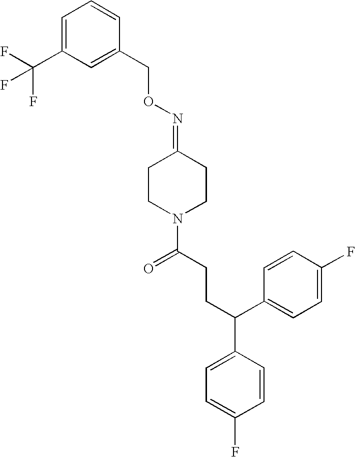

Piperidin-4-one O-(3-trifluoromethylbenzyl)oxime hydrochloride (247 mg, 0.80 mmol) and 4,4-bis-(4-fluorophenyl)butyric acid (243 mg, 0.88 mmol) were dissolved in dichloromethane (4 ml). 1-Hydroxybenzotriazole (108 mg, 0.80 mmol), N-ethyldimethylaminopropylcarbodiimide hydrochloride (184 mg, 0.96 mmol) and triethylamine (0.135 ml, 0.96 mmol) were added to the mixture and stirred at room temperature for 1 hour. Saturated aqueous NaHCO₃ solution (10 ml) was added, extracted with chloroform (20 ml×2), dried (MgSO₄) and evaporated under reduced pressure. The residue was purified by column chromatography on silica (ethyl acetate/hexane, 35/65-60/40) to give the title compound (420 mg, 99%).

¹H—NMR (DMSO-d₆) δ: 7.72-7.58 (4H, m), 7.38-7.24 (4H, m), 7.18-7.02 (4H, m), 5.10 (2H, s), 4.04-3.98 (1H, m), 3.60-3.30 (4H, m), 2.54-2.42 (2H, m), 2.30-2.18 (6H, m).

### Example 30

### 1-[4,4-Bis-(4-fluorophenyl)butyryl]piperidin-4-one O-(4-trifluoromethylbenzyl)oxime

was prepared as described in EXAMPLE 29

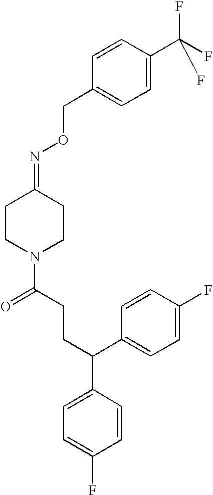

¹H-NMR (DMSO-d₆) δ: 7.63 (4H, dd), 7.21 (8H, dt), 5.13 (2H, s), 4.03 (1H, s), 3.44 (6H, t), 2.51 (3H, s), 2.24 (6H, s).

### Example 31

### 1-[4,4-Bis-(4-fluorophenyl)butyryl]piperidin-4-one O-benzyloxime

was prepared as described in EXAMPLE 29

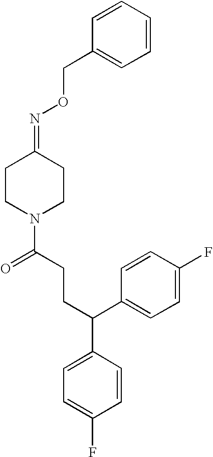

¹H-NMR (DMSO-d₆) δ: 7.57 (6H, dd), 5.03 (1H, s), 3.49 (5H, dd), 2.73-2.31 (6H, m), 1.76 (1H, t), 1.48 (2H, dt), 0.87 (6H, t).

### Example 32

### 1-[4,4-Bis-(4-fluorophenyl)butyl]piperidin-4-one O-benzyloxime oxalate

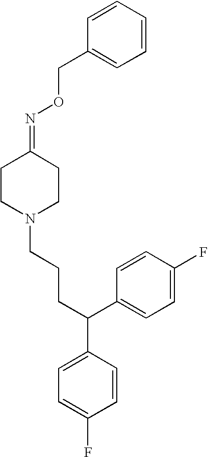

Piperidin-4-one O-benzyloxime hydrochloride (100 mg, 0.49 mmol) and 1,1′-(4-chlorobutylidene)-bis-(4-fluorobenzene) (144 mg, 0.51 mmol) was dissolved in N,N-dimethylformamide (3 ml). KI (8 mg, 0.05 mmol) and K₂CO₃ (203 mg, 1.47 mmol) were added to the mixture and heated for overnight. After quenching with H₂O (3 ml), the mixture was extracted with ethyl acetate (30 ml×3), washed with brine (×2), dried over Na₂SO₄ and evaporated under reduced pressure. The residue was purified by column chromatography on silica (ethyl acetate/hexane, 1/9-2/3) to give the desired compound as the pale yellow oil. To a solution of this free base in ethanol (1 ml), a solution of oxalic acid (23 mg, 0.255 mmol) in ethanol (1 ml) was added. The resulting white precipitate was collected, washed with diethyl ether (2 ml) and dried under reduced pressure at 70° C. to give the title compound.

### Example 33

### 1-[4,4-Bis-(4-fluorophenyl)butyl]piperidin-4-one O-(4-trifluoromethylbenzyl)oxime oxalate

was prepared as described in EXAMPLE 32

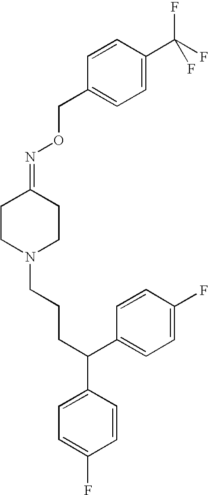

### Example 34

### 1-(3-Isobutylaminopropionyl)piperidin-4-one O-(3-trifluoromethylbenzyl)oxime hydrochloride

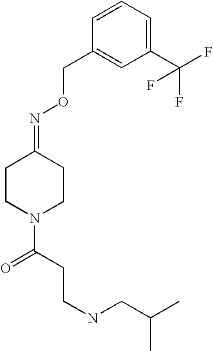

a) To a solution of piperidin-4-one O-(3-trifluoromethylbenzyl)oxime hydrochloride (946 mg, 3.63 mmol) and acryl chloride (0.315 ml, 3.88 mmol) in dichloromethane was added pyridine (0.313 ml, 3.88 mmol) at 0° C. and stirred at room temperature for 30 min. After quenching with H₂O (3 ml), the mixture was extracted by chloroform/H₂O (×3), dried over Na₂SO₄ and concentrated in vacuo. The residue was purified by column chromatography on silica (ethyl acetate/hexane, 1/3-4/1) to give 1-but-3-enoylpiperidin-4-one O-(3-trifluoromethylbenzyl)oxime (500 mg, 42%, colorless oil).

b) 1-But-3-enoylpiperidin-4-one O-(3-trifluoromethylbenzyl)oxime (80 mg, 0.25 mmol), isobutylamine (0.248 ml, 2.5 mmol) and ethanol (1.5 ml) were mixed together in a sealed tube and heated at 150° C. with microwave equipment for 30 min. The reaction mixture was evaporated in vacuo and the residue was purified by column chromatography on silica gel (chloroform/methanol/NH₃aq.=100/10/1) to afford the desired compound as a free base, which was dissolved in ethyl acetate (2 ml) and treated with 4N—HCl in ethyl acetate (2 ml) solution. The resulting mixture was concentrated in vacuo and dried under reduced pressure for 24 hours at 70° C. to afford the title compound.

### Example 35

### 1-[3-(4-Hydroxypiperidin-1-yl)propionyl]piperidin-4-one O-(3-trifluoromethylbenzyl)oxime hydrochloride

was prepared as described in EXAMPLE 34

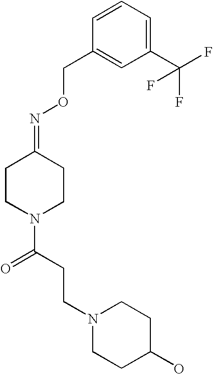

### Example 36

### 1-[3-(4-Hydroxycyclohexylamino)propionyl]piperidin-4-one O-(3-trifluoromethylbenzyl)oxime hydrochloride

was prepared as described in EXAMPLE 34

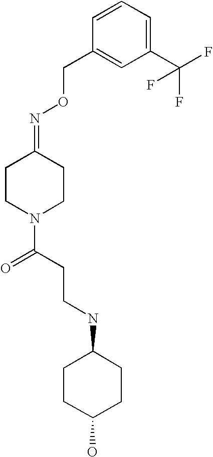

### Example 37

### 1-[3-(2-Hydroxyethylamino)propionyl]piperidin-4-one O-(3-trifluoromethylbenzyl)oxime

was prepared as described in EXAMPLE 34

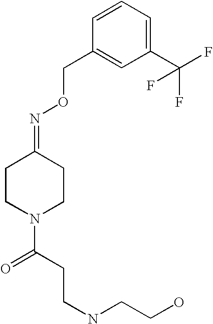

### Example 38

### 1-[3-(3-Hydroxypropylamino)propionyl]piperidin-4-one O-(3-trifluoromethylbenzyl)oxime

was prepared as described in EXAMPLE 34

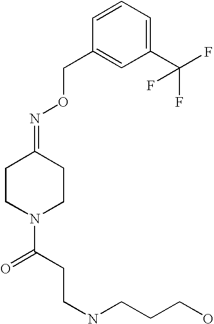

### Example 39

### 1-{3-[(2-Hydroxyethyl)methylamino]propionyl}piperidin-4-one O-(3-trifluoromethylbenzyl)oxime

was prepared as described in EXAMPLE 34

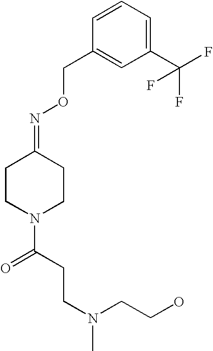

### Example 40

### 1-[3-(2-Morpholin-4-ylethylamino)propionyl]piperidin-4-one O-(3-trifluoromethylbenzyl)oxime hydrochloride

was prepared as described in EXAMPLE 34

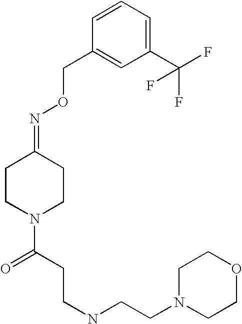

### Example 41

### 1-(3-Morpholin-4-ylpropionyl)piperidin-4-one O-(3-trifluoromethylbenzyl)oxime hydrochloride

was prepared as described in EXAMPLE 34

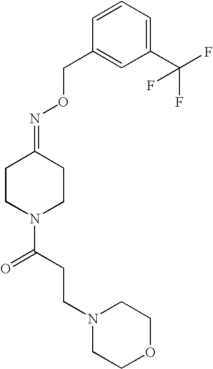

### Example 42

### 1-[3-(4-Hydroxy-4-phenylpiperidin-1-yl)propionyl]-piperidin-4-one O-(3-trifluoromethylbenzyl)oxime hydrochloride

was prepared as described in EXAMPLE 34

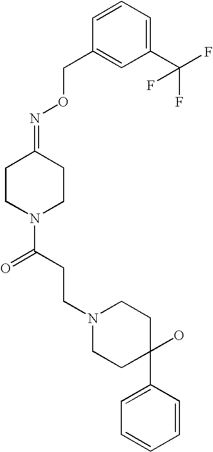

### Example 43

### 1-[3-(4-Methylpiperazin-1-yl)propionyl]piperidin-4-one O-(3-trifluoromethylbenzyl)oxime dihydrochloride

was prepared as described in EXAMPLE 34

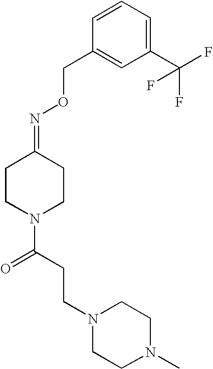

### Example 44

### 1-[3-(4-Phenylpiperazin-1-yl)propionyl]piperidin-4-one O-(3-trifluoromethylbenzyl)oxime dihydrochloride

was prepared as described in EXAMPLE 34

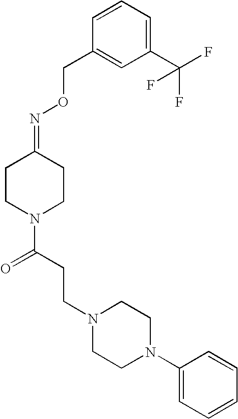

### Example 45

### 1-[3-(4-Acetylpiperazin-1-yl)propionyl]piperidin-4-one O-(3-trifluoromethylbenzyl)oxime

was prepared as described in EXAMPLE 34

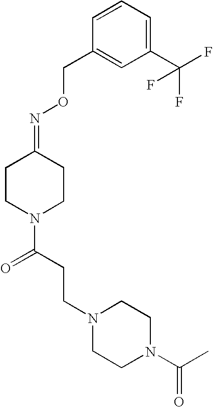

### Example 46

### 1-{3-[4-(2-Hydroxyethyl)piperazin-1-yl]propionyl}piperidin-4-one O-(3-trifluoromethylbenzyl)oxime dihydrochloride

was prepared as described in EXAMPLE 34

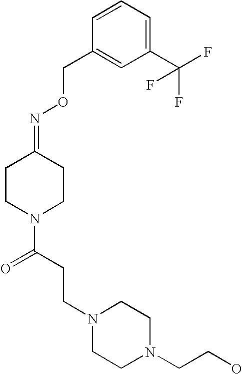

### Example 47

### 1-[3-(4-Phenylpiperidin-1-ylpropionyl]piperidin-4-one O-(3-trifluoromethylbenzyl)oxime hydrochloride

was prepared as described in EXAMPLE 34

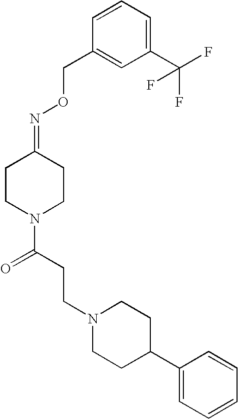

¹H-NMR (DMSO-d₆) δ: 11.00 (1H, s), 7.68-7.60 (4H, m), 7.29 (5H, d), 5.15 (2H, s), 3.59 (6H, t), 3.12-3.03 (4H, m), 2.86-1.92 (10H, m).

### Example 48

### 1-[3-(4-Benzylpiperidin-1-yl)propionyl]piperidin-4-one O-(3-trifluoromethylbenzyl)oxime hydrochloride

was prepared as described in EXAMPLE 34

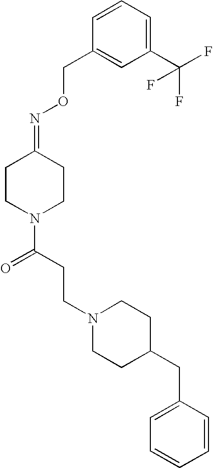

¹H-NMR (DMSO-d₆) δ: 10.60 (1H, s), 7.64 (4H, dd), 7.25 (5H, d), 5.14 (2H, s), 3.56-3.20 (12H, m), 2.97-2.29 (10H, m), 1.96-1.56 (5H, m).

### Example 49

### 1-(2-Morpholin-4-ylethanesulfonyl)piperidin-4-one O-(3-trifluoromethylbenzyl)oxime

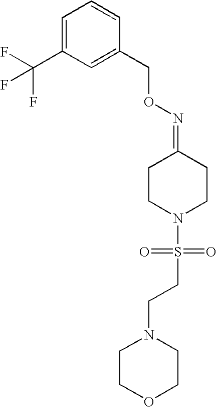

a) To a solution of piperidin-4-one O-(3-trifluoromethylbenzyl)oxime (100 mg, 0.37 mmol) in dichloromethane (5 ml), 2-chloroethansulfonyl chloride (120 mg, 0.74 mmol) and triethylamine (0.103 ml, 0.74 mmol) in dichloromethane (3 ml) was slowly added to this mixture at 0° C. and stirred for 15 minutes. Saturated aqueous NaHCO₃ solution (10 ml) was added, extracted with chloroform (20 ml×2), dried (MgSO₄) and evaporated under reduced pressure. The residue was purified by column chromatography on silica (ethyl acetate/hexane, 25/75-50/50) to afford 1-ethenesulfonylpiperidin-4-one O-(3-trifluoromethylbenzyl)oxime (106 mg, 80%, colorless oil).

b) 1-Ethenesulfonyl-piperidin-4-one O-(3-trifluoromethyl-benzyl)-oxime (93 mg, 0.27 mmol) was dissolved in ethanol (1 ml). Morpholine (0.112 ml, 1.28 mmol) was added to this mixture and stirred at room temperature for 12 hours. The reaction mixture was concentrated under reduced pressure. Then residue was diluted with ethyl acetate (30 ml), washed with H₂O and brine. The organic layer was dried over MgSO₄ and concentrated to dryness to afford the title compound (120 mg, 100%).

### Example 50

### 1-(4-Butyl-benzenesulfonyl)piperidin-4-one O-(3-trifluoromethylbenzyl)oxime

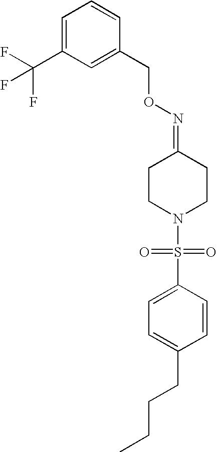

To a solution of piperidin-4-one O-(3-trifluoromethylbenzyl)oxime hydrochloride (247 mg, 0.80 mmol) and triethylamine (0.246 ml, 1.76 mmol) in dichloromethane (4 ml), a solution of 4-butylbenzenesulfonyl chloride (243 mg, 0.88 mmol) in dichloromethane (1 ml) was slowly added at 0° C. and stirred at room temperature for 30 minutes. Saturated aqueous NaHCO₃ solution (10 ml) was added, extracted with chloroform (20 ml×2), dried (MgSO₄) and evaporated under reduced pressure. The residue was purified by column chromatography on silica (ethyl acetate/hexane, 10/90-40/60) to give the title compound (345 mg, 92%).

¹H-NMR (DMSO-d₆) δ: 7.62-7.49 (8H, m), 5.07 (2H, s), 3.06-2.99 (4H, m), 2.68-2.63 (4H, m), 2.32 (2H, t), 1.63-1.53 (2H, m), 1.33-1.28 (2H, m), 0.90 (3H, t).

### Example 51

### 1-Benzenesulfonylpiperidin-4-one O-(3-trifluoromethylbenzyl)oxime

was prepared as described in EXAMPLE 50

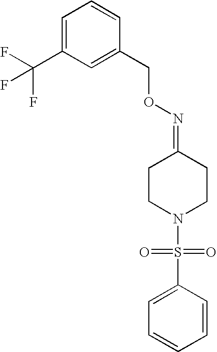

¹H-NMR (DMSO-d₆) δ: 7.80-7.50 (9H, m), 5.08 (2H, s), 3.12-3.00 (4H, m), 2.64 (2H, t), 2.31 (2H, t).

### Example 52

### 1-Methanesulfonylpiperidin-4-one O-(3-trifluoromethylbenzyl)oxime

was prepared as described in EXAMPLE 50

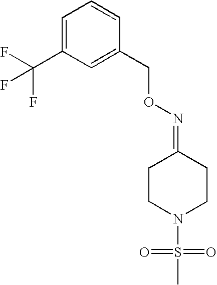

### Example 53

### 4-[4-(3-Trifluoromethylbenzyloxyimino)piperidine-1-sulfonyl]benzonitrile

was prepared as described in EXAMPLE 50

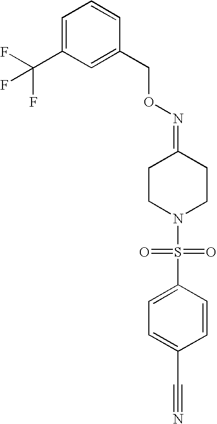

¹H-NMR (DMSO-d₆) δ: 8.18 (2H, d), 7.97 (2H, d), 7.70-7.56 (4H, m), 5.08 (2H, s), 3.20-3.06 (4H, m), 2.63 (2H, t), 2.36 (2H, t).

### Example 54

### 1-(1-Methyl-1H-imidazole-4-sulfonyl)piperidin-4-one O-(3-trifluoromethylbenzyl)oxime

was prepared as described in EXAMPLE 50

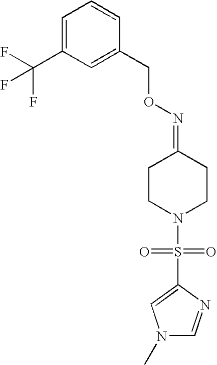

### Example 55

### 1-(4-Trifluoromethylbenzenesulfonyl)piperidin-4-one O-(3-trifluoromethylbenzyl)oxime

was prepared as described in EXAMPLE 50

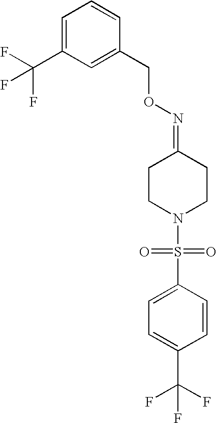

### Example 56

### 1-(3-Trifluoromethylbenzenesulfonyl)piperidin-4-one O-(3-trifluoromethylbenzyl)oxime

was prepared as described in EXAMPLE 50

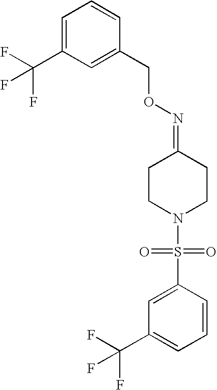

¹H-NMR (DMSO-d₆) δ: 8.18-8.06 (2H, m), 7.99 (1H, s), 7.90 (1H, t), 7.64-7.52 (4H, m), 5.06 (2H, s), 3.20-3.08 (4H, m), 2.64 (2H, t), 2.34 (2H, t).

### Example 57

### 1-(2-Trifluoromethylbenzenesulfonyl)piperidin-4-one O-(3-trifluoromethylbenzyl)oxime

was prepared as described in EXAMPLE 50

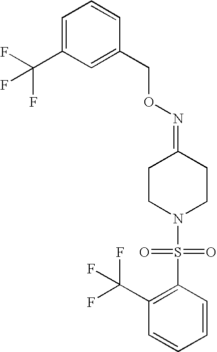

### Example 58

### 1-(4-Trifluoromethoxybenzenesulfonyl)piperidin-4-one O-(3-trifluoromethylbenzyl)oxime

was prepared as described in EXAMPLE 50

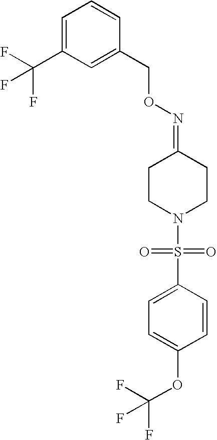

¹H-NMR (DMSO-d₆) δ: 7.92 (2H, d), 7.70-7.52 (6H, m), 5.04 (2H, s), 3.14-3.02 (4H, m), 2.64 (2H, t), 2.32 (2H, t).

### Example 59

### 1-(3-Trifluoromethoxybenzenesulfonyl)piperidin-4-one O-(3-trifluoromethylbenzyl)oxime

was prepared as described in EXAMPLE 50

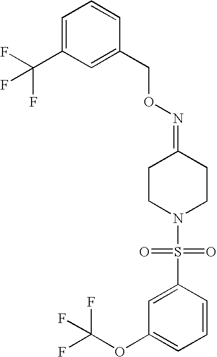

¹H-NMR (DMSO-d₆) δ: 7.84-7.78 (3H, m), 7.72 (1H, s), 7.68-7.56 (4H, m), 5.08 (2H, s), 3.18-3.06 (4H, m), 2.62 (2H, t), 2.34 (2H, t).

### Example 60

### 1-(2-Trifluoromethoxybenzenesulfonyl)piperidin-4-one O-(3-trifluoromethylbenzyl)oxime

was prepared as described in EXAMPLE 50

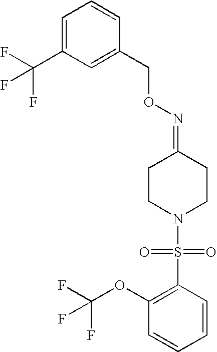

### Example 61

### 1-(4-Methoxybenzenesulfonyl)piperidin-4-one O-(3-trifluoromethylbenzyl)oxime

was prepared as described in EXAMPLE 50

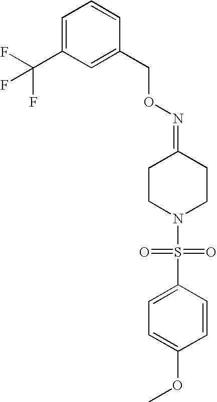

### Example 62

### 1-(4-Fluorobenzenesulfonyl)piperidin-4-one O-(3-trifluoromethylbenzyl)oxime

was prepared as described in EXAMPLE 50

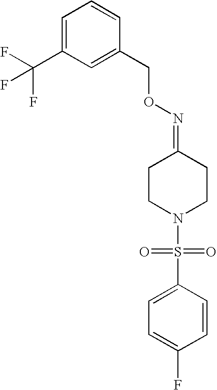

¹H-NMR (DMSO-d₆) δ: 7.92-7.80 (2H, m), 7.72-7.42 (6H, m), 5.06 (2H, s), 3.16-2.98 (4H, m), 2.63 (2H, t), 2.32 (2H, t).

### Example 63

### 1-(2-Fluorobenzenesulfonyl)piperidin-4-one O-(3-trifluoromethylbenzyl)oxime

was prepared as described in EXAMPLE 50

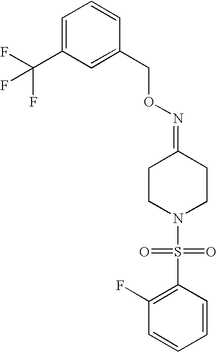

¹H-NMR (DMSO-d₆) δ: 7.82-7.68 (2H, m), 7.64-7.38 (6H, m), 5.06 (2H, s), 3.28-3.16 (4H, m), 2.62 (2H, t), 2.33 (2H, t).

### Example 64

### 1-(6-Chloropyridine-3-sulfonyl)piperidin-4-one O-(3-trifluoromethylbenzyl)oxime

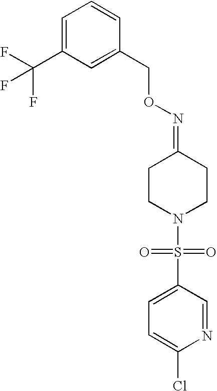

To a solution of piperidin-4-one O-(3-trifluoromethylbenzyl)oxime (1.09 g, 4.0 mmol) and triethylamine (1.23 ml, 8.8 mmol) in dichloromethane (15 ml), 6-chloropyridine-3-sulfonyl chloride (933 mg, 4.4 mmol) in dichloromethane (5 ml) was slowly added at 0° C. and stirred for 30 minutes. Saturated aqueous NaHCO₃ solution (15 ml) was added, extracted with chloroform (50 ml×2), dried (MgSO₄) and evaporated under reduced pressure. The residue was purified by column chromatography on silica (ethyl acetate/hexane, 20/80-45/55) to afford the title compound (1.75 g, 98%).

¹H-NMR (DMSO-d₆) δ: 8.78 (1H, d), 8.21 (1H, dd), 7.80 (1H, d), 7.68-7.50 (4H, m), 5.04 (2H, s), 3.22-3.04 (4H, m), 2.63 (2H, t), 2.36 (2H, t).

### Example 65

### 1-(4-Butylbenzenesulfonyl)piperidin-4-one O-(3-chlorobenzyl)oxime

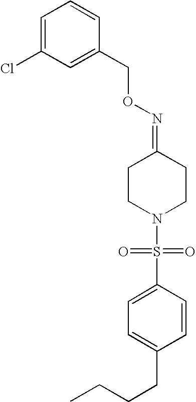

a) To a suspension of 4-piperidone monohydrate hydrochloride (1.10 g, 7.00 mmol) in dichloromethane (50 ml), N,N-diisopropylethylamine (7.71 ml, 45.0 mmol) and a solution of p-butylbenzenesulfonyl chloride (3.49 g, 15.0 mmol) in dichloromethane (25 ml) were added, and the mixture was stirred at room temperature for 7 hours. Saturated aqueous NaHCO₃ solution (20 ml) was added, extracted with chloroform (50 ml×2), dried (MgSO₄) and evaporated under reduced pressure. The residue was purified by column chromatography on silica (ethyl acetate/hexane, 25/75-45/55) to afford 1-(4-butylbenzenesulfonyl)piperidine-4,4-diol (3.88 g, 83%, yellow solid).

b) A suspension of 1-(4-butyl-benzenesulfonyl)piperidine-4,4-diol (1.57 g, 5.0 mmol), hydroxyammonium chloride (417 mg, 6.0 mmol) and sodium acetate (492 mg, 6.0 mmol) in ethanol (20 ml) was stirred at room temperature for 2 hours. The reaction mixture was concentrated under reduced pressure. Saturated aqueous NaHCO₃ solution (25 ml) was added, extracted with ethyl acetate (80 ml×2), washed with brine dried (MgSO₄) and evaporated under reduced pressure. The residue was recrystallized from ethyl acetate/hexane to give 1-(4-butylbenzenesulfonyl)piperidin-4-one oxime (1.02 g, 66%, colorless solid).

c) To a solution of 1-(4-butylbenzenesulfonyl)piperidin-4-one oxime (94 mg, 0.30 mmol) and 3-chlorobenzyl bromide (74 mg, 0.36 mmol) in tetrahydrofuran (4 ml), NaH was added under nitrogen atmosphere and stirred at 60° C. for 1 hour. The reaction was quenched with H₂O (5 ml), extracted with ethyl acetate (20 ml×2), washed with brine, dried (MgSO₄) and evaporated under reduced pressure. The residue was purified by column chromatography on silica (ethyl acetate/hexane, 20/80-40/60) to afford the title compound (120 mg, 92%).

¹H-NMR (DMSO-d₆) δ: 7.63 (2H, d), 7.48 (2H, d), 7.38-7.20 (4H, m), 4.98 (2H, s), 3.12-2.98 (4H, m), 2.72-2.59 (4H, m), 2.32 (2H, t), 1.58 (2H, quintet), 1.32 (2H, dt), 0.90 (3H, t).

### Example 66

### 1-(4-Butylbenzenesulfonyl)piperidin-4-one O-(4-trifluoromethylbenzyl)oxime

was prepared as described in EXAMPLE 65

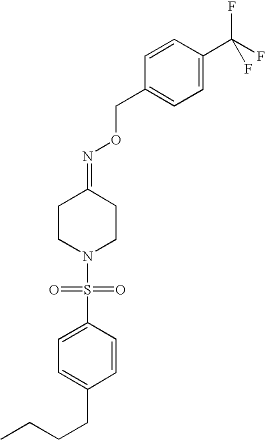

### Example 67

### 1-(4-Butylbenzenesulfonyl)piperidin-4-one O-benzyloxime

was prepared as described in EXAMPLE 65

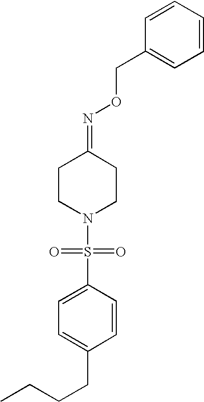

¹H-NMR (DMSO-d₆) δ: 7.65 (2H, d), 7.37 (7H, t), 4.96 (2H, s), 3.03 (4H, d), 2.67 (2H, s), 2.50 (1H, s), 2.31 (2H, s), 1.56 (2H, s), 1.29 (2H, d), 0.88 (3H, s).

### Example 68

### 1-(4-Butylbenzenesulfonyl)piperidin-4-one O-pyridin-2-ylmethyloxime

was prepared as described in EXAMPLE 65

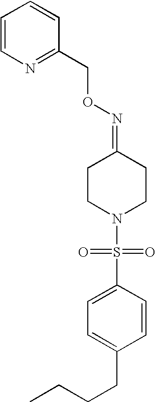

¹H-NMR (DMSO-d₆) δ: 8.44 (1H, dd), 7.72 (1H, dt), 7.63 (2H, d), 7.42 (2H, d), 7.30-7.21 (2H, m), 5.02 (2H, s), 3.10-3.01 (4H, m), 2.73-2.60 (4H, m), 2.31 (2H, t), 1.56 (2H, quintet), 1.28 (2H, dt), 0.90 (3H, t).

### Example 69

### 1-(4-Butylbenzenesulfonyl)piperidin-4-one O-(2-chloropyridin-4-ylmethyl)oxime

was prepared as described in EXAMPLE 65.

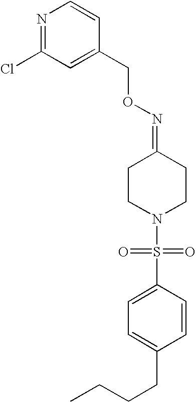

¹H-NMR (DMSO-d₆) δ: 8.36 (1H, d), 7.68 (2H, d), 7.46 (2H, d), 7.34 (1H, s), 7.28 (1H, d), 5.02 (2H, s), 3.11-3.01 (4H, m), 2.78-2.60 (4H, m), 2.28-2.23 (2H, m), 1.56 (2H, quintet), 1.30 (2H, dt), 0.90 (3H, t).

### Example 70

### 1-(4-Butylbenzenesulfonyl)piperidin-4-one O-pyridin-3-ylmethyloxime

was prepared as described in EXAMPLE 65.

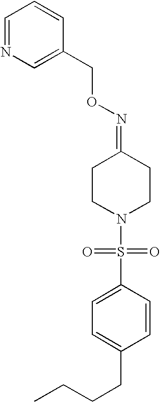

¹H-NMR (DMSO-d₆) δ: 8.60-8.42 (2H, m), 7.81-7.61 (3H, m), 7.59-7.30 (3H, m), 5.03 (2H, s), 3.18-2.94 (4H, m), 2.76-2.50 (4H, m), 2.40-2.28 (2H, m), 1.58 (2H, quintet), 1.31 (2H, dt), 0.90 (3H, t).

### Example 71

### 1-(4-Butylbenzenesulfonyl)piperidin-4-one O-pyridin-4-ylmethyloxime

was prepared as described in EXAMPLE 65.

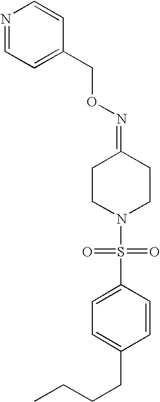

¹H-NMR (DMSO-d₆) δ: 8.50 (2H, d), 7.68 (2H, d), 7.48 (2H, d), 7.23 (2H, d), 5.03 (2H, s), 3.13-3.01 (4H, m), 2.76-2.62 (4H, m), 2.38-2.24 (2H, m), 1.59 (2H, quintet), 1.32 (2H, dt), 0.92 (3H, t).

### Example 72

### 1-(4-Butylbenzenesulfonyl)piperidin-4-one O-(4-chlorobenzyl)oxime

was prepared as described in EXAMPLE 65.

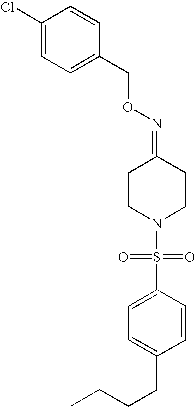

¹H-NMR (DMSO-d₆) δ: 7.64 (2H, d), 7.48 (2H, d), 7.38 (2H, d), 7.30 (2H, d), 4.98 (2H, s), 3.12-2.98 (4H, m), 2.74-2.58 (4H, m), 2.32 (2H, t), 1.58 (2H, quintet), 1.31 (2H, dt), 0.92 (3H, t).

### Example 73

### 1-(4-Butylbenzenesulfonyl)piperidin-4-one O-(4-chloropyridin-2-ylmethyl)oxime

was prepared as described in EXAMPLE 65.

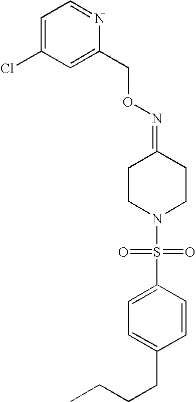

¹H-NMR (DMSO-d₆) δ: 8.51 (1H, d), 7.68 (2H, d), 7.52-7.40 (3H, m), 7.36 (1H, s), 5.03 (2H, s), 3.13-3.01 (4H, m), 2.74-2.62 (4H, m), 2.34 (2H, t), 1.58 (2H, quintet), 1.32 (2H, dt), 0.91 (3H, t).

### Example 74

### 1-(4-Butylbenzenesulfonyl)piperidin-4-one O-[1-(3-trifluoromethylphenyl)ethyl]oxime

was prepared as described in EXAMPLE 65.

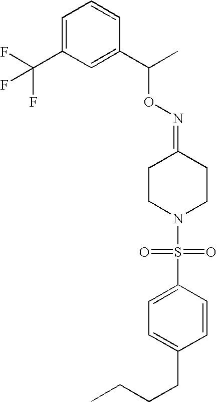

¹H-NMR (DMSO-d₆) δ: 7.70-7.41 (8H, m), 5.21 (1H, q), 3.22-3.02 (2H, m), 2.92-2.80 (2H, m), 2.79-2.54 (4H, m), 2.37-2.19 (2H, m), 1.58 (2H, quintet), 1.41 (3H, d), 1.36 (2H, dt), 0.90 (3H, t).

### Example 75

### 1-(4-Butylbenzenesulfonyl)piperidin-4-one O-(6-chloropyridin-2-ylmethyl)oxime

was prepared as described in EXAMPLE 65

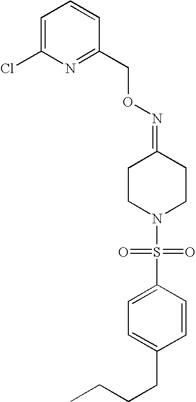

¹H-NMR (DMSO-d₆) δ: 7.82 (1H, t), 7.68 (2H, d), 7.48 (2H, d), 7.39 (1H, d), 7.31 (1H, d), 5.00 (2H, s), 3.12-3.02 (4H, m), 2.70-2.62 (4H, m), 2.32 (2H, t), 1.58 (2H, quintet), 1.30 (2H, dt), 0.90 (3H, t).

### Example 76

### 1-(4-Butylbenzenesulfonyl)piperidin-4-one O-(6-methoxypyridin-2-ylmethyl)oxime

was prepared as described in EXAMPLE 65.

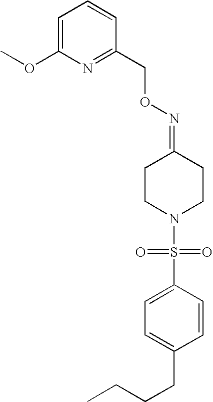

¹H-NMR (DMSO-d₆) δ: 7.68 (2H, d), 7.61 (2H, d), 7.48 (2H, d), 6.88 (1H, d), 6.63 (1H, d), 4.98 (2H, s), 3.80 (3H, s), 3.12-3.02 (4H, m), 2.73-2.63 (4H, m), 2.34 (2H, t), 1.58 (2H, quintet), 1.32 (2H, dt), 0.91 (3H, t).

### Example 77

### 3-[1-(4-Butylbenzenesulfonyl)piperidin-4-ylideneaminooxymethyl]benzonitrile

was prepared as described in EXAMPLE 65

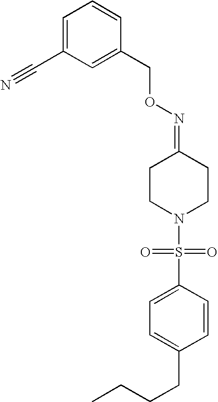

¹H-NMR (DMSO-d₆) δ: 7.78-7.42 (8H, m), 5.01 (2H, s), 3.12-2.98 (4H, m), 2.72-2.58 (4H, m), 2.33 (2H, t), 1.58 (2H, quintet), 1.32 (2H, dt), 0.88 (3H, t).

### Example 78

### 1-(4-Butylbenzenesulfonyl)piperidin-4-one O-(2-trifluoromethylbenzyl)oxime

was prepared as described in EXAMPLE 65.

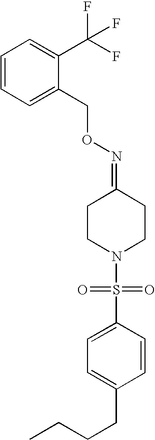

¹H-NMR (DMSO-d₆) δ: 7.78-7.42 (8H, m), 5.13 (2H, s), 3.12-3.00 (4H, m), 2.74-2.60 (4H, m), 2.32 (2H, t), 1.58 (2H, quintet), 1.31 (2H, dt), 0.89 (3H, t).

### Example 79

### 1-(4-Butylbenzenesulfonyl)piperidin-4-one O-(3-trifluoromethoxybenzyl)oxime

was prepared as described in EXAMPLE 65

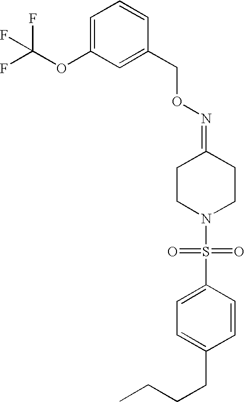

¹H-NMR (DMSO-d₆) δ: 7.64 (2H, d), 7.50-7.40 (3H, m), 7.38-7.20 (3H, m), 5.02 (2H, s), 3.12-2.98 (4H, m), 2.72-2.60 (4H, m), 2.32 (2H, t), 1.58 (2H, quintet), 1.32 (2H, dt), 0.92 (3H, t).

### Example 80

### 1-(4-Butylbenzenesulfonyl)piperidin-4-one O-(3-methoxybenzyl)oxime

was prepared as described in EXAMPLE 65

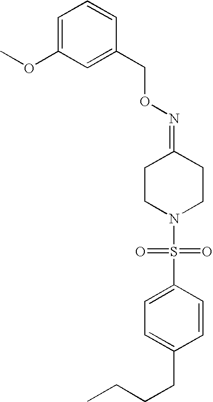

¹H-NMR (DMSO-d₆) δ: 7.68 (2H, d), 7.42 (2H, d), 7.22 (1H, t), 6.90-6.80 (3H, m), 4.96 (2H, s), 3.72 (3H, s), 3.12-2.98 (4H, m), 2.72-2.58 (4H, m), 2.32 (2H, t), 1.58 (2H, quintet), 1.32 (2H, dt), 0.90 (3H, t).

### Example 81

### 1-(4-Butylbenzenesulfonyl)piperidin-4-one O-(4-fluoro-3-trifluoromethylbenzyl)oxime

was prepared as described in EXAMPLE 65.

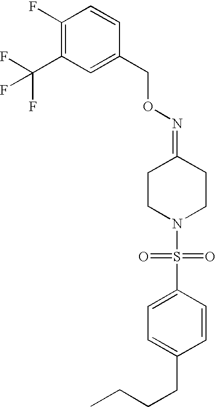

¹H-NMR (DMSO-d₆) δ: 7.72-7.62 (4H, m), 7.50-7.42 (3H, m), 5.02 (2H, s), 3.10-2.98 (4H, m), 2.72-2.58 (4H, m), 2.32 (2H, t), 1.58 (2H, quintet), 1.30 (2H, dt), 0.90 (3H, t).

### Example 82

### 1-(4-Butylbenzenesulfonyl)piperidin-4-one O-cyclohexylmethyloxime

was prepared as described in EXAMPLE 65

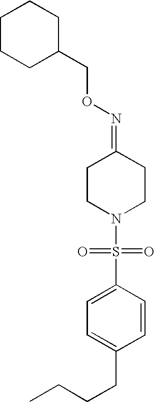

¹H-NMR (DMSO-d₆) δ: 7.62 (2H, d), 7.42 (2H, d), 3.68 (2H, d), 3.10-2.92 (4H, m), 2.62 (2H, t), 2.50 (2H, t), 2.18 (2H, t), 1.70-1.43 (8H, m), 1.38-1.00 (5H, m), 0.96-0.78 (5H, m).

### Example 83

### 1-(4-Aminobenzenesulfonyl)piperidin-4-one O-(3-trifluoromethylbenzyl)oxime hydrochloride

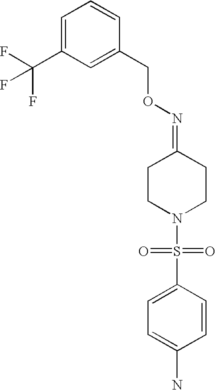

a) To a solution of 4-piperidone monohydrate hydrochloride (2.38 g, 15.0 mmol) in pyridine (15 ml) and dichloromethane (5 ml), 4-nitrobenzenesulfonyl chloride (3.49 g, 16.5 mmol) in dichloromethane (10 ml) was slowly added at 0° C. and stirred at room temperature for 2.5 hours. The solvent was evaporated under reduced pressure and the residue was diluted with ethyl acetate (10 ml). HCl (1 M, 50 ml) was added, extracted with chloroform (100 ml×2), dried (MgSO₄) and evaporated under reduced pressure. The residue was purified by column chromatography on silica (chloroform/methanol, 100/0-90/20) and recrystallized from ethyl acetate/hexane to give 1-(4-nitrobenzenesulfonyl)piperidine-4,4-diol (1.83 g, 40%, pale yellow solid).

b) A suspension of 1-(4-nitrobenzenesulfonyl)piperidine-4,4-diol (351 mg, 1.16 mmol), O-(3-trifluoromethylbenzyl)hydroxylamine hydrochloride (264 mg, 1.16 mmol) and sodium acetate (95 mg, 1.16 mmol) in ethanol (12 ml) was stirred at room temperature for 1 hour. The solvent was evaporated under reduced pressure. Saturated aqueous NaHCO₃ solution (10 ml) was added, extracted with chloroform (30 ml×2), dried (MgSO₄) and evaporated under reduced pressure to give 1-(4-nitrobenzenesulfonyl)piperidine-4-one O-(3-trifluoromethylbenzyl)oxime (530 mg, 100%, colorless solid).

c) 1-(4-Nitrobenzenesulfonyl)piperidine-4-one O-(3-trifluoromethylbenzyl)oxime (530 mg, 1.16 mmol) was taken up in toluene (10 ml). To the mixture, Fe (324 mg, 5.8 mmol), NH₄Cl (620 mg, 11.6 mmol) and H₂O (1 ml) was added. The reaction mixture was heated at 115° C. and stirred for 3 hours. The reaction mixture was diluted with 30 ml of ethyl acetate, then filtrated through a pad of Celite. The filtrate was concentrated under reduced pressure. The residue was purified by column chromatography on silica (ethyl acetate/hexane, 30/70-50/50) to afford 1-(4-aminobenzenesulfonyl)piperidin-4-one O-(3-trifluoromethylbenzyl)oxime (471 mg, 95%, pale yellow oil). To a solution of the free base in ethyl acetate, 4N—HCl in ethyl acetate was added. The resulting precipitate was collected and dried in vacuo to give the title compound.

### Example 84

### 1-(3-Aminobenzenesulfonyl)piperidin-4-one O-(3-trifluoromethylbenzyl)oxime

was prepared as described in EXAMPLE 83

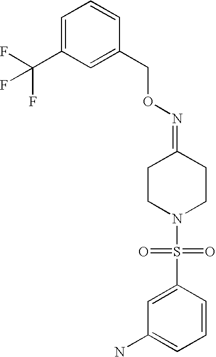

### Example 85

### 1-(2-Aminobenzenesulfonyl)piperidin-4-one O-(3-trifluoromethylbenzyl)oxime

was prepared as described in EXAMPLE 83

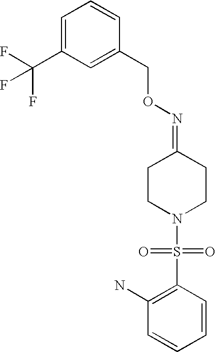

¹H-NMR (DMSO-d₆) δ: 7.68-7.50 (4H, m), 7.41 (1H, d), 7.32 (1H, t), 6.86 (1H, d), 6.62 (1H, t), 6.08 (2H, s), 5.10 (2H, s), 3.22-3.10 (4H, m), 2.60 (2H, t), 2.28 (2H, t).

### Example 86

### 1-[4-(2-Hydroxyethylamino)benzenesulfonyl]piperidin-4-one O-(3-trifluoromethylbenzyl)oxime hydrochloride

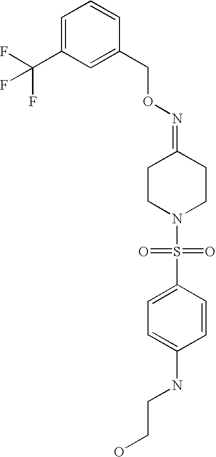

1-(4-Aminobenzenesulfonyl)piperidin-4-one O-(3-trifluoromethylbenzyl)oxime (145 mg, 0.34 mmol) and pyridine (35 mg, 0.45 mmol) was taken up in 1 ml of chloroform. Chloroformic acid 2-chloroethyl ester (49 mg, 0.34 mmol) in chloroform (1 ml) was added over 5 minutes, and stirred at room temperature for 10 minutes. The solvent was evaporated under reduced pressure. The residue was dissolve in ethanol (6 ml) and H₂O (2 ml). To the mixture, KOH (152 mg, 2.72 mmol) was added. The mixture was heated at 100° C. and stirred for 15 hours. The reaction mixture was concentrated under reduced pressure. The residue was dissolved in ethyl acetate (40 ml) and the mixture was washed with H₂O and brine. The organic layer was dried over MgSO₄ and concentrated in vacuo. The residue was purified by column chromatography on silica (ethyl acetate/methanol, 100/0-90/10) to afford 1-[4-(2-hydroxyethylamino)benzenesulfonyl]piperidin-4-one O-(3-trifluoromethylbenzyl)oxime (112 mg, 70%, colorless oil). To a solution of the free base in ethyl acetate (1 ml), 4N—HCl in ethyl acetate (1 ml) was added. The solvent was removed under reduced pressure. The residue was triturated with ether (10 ml) to give the title compound.

### Example 87

### 1-[3-(2-Hydroxyethylamino)benzenesulfonyl]piperidin-4-one O-(3-trifluoromethylbenzyl)oxime hydrochloride

was prepared as described in EXAMPLE 86

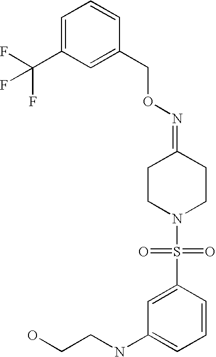

### Example 88

### 1-[2-(2-Hydroxyethylamino)benzenesulfonyl]piperidin-4-one O-(3-trifluoromethylbenzyl)oxime hydrochloride

was prepared as described in EXAMPLE 86

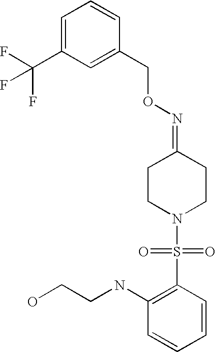

### Example 89

### 2-Amino-N-{4-[4-(3-trifluoromethylbenzyloxyimino)piperidine-1-sulfonyl]phenyl}acetamide hydrochloride

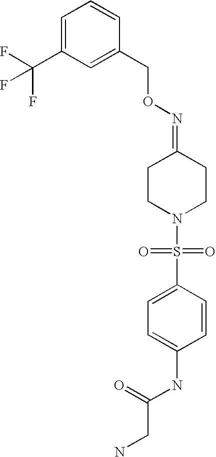

a) To a solution of 1-(4-aminobenzenesulfonyl)piperidin-4-one O-(3-trifluoromethylbenzyl)oxime hydrochloride (125 mg, 0.29 mmol) and Boc-glycine (61 mg, 0.35 mmol) in dichloromethane (5 ml), 1-hydroxybenzotriazole (40 mg, 0.29 mmol) and N-ethyldimethylaminopropylcarbodiimide hydrochloride (167 mg, 0.35 mmol) were added and stirred at room temperature for 36 hours. Saturated aqueous NaHCO₃ solution (10 ml) was added, extracted with chloroform (20 ml×2), dried (MgSO₄) and evaporated under reduced pressure. The residue was purified by column chromatography on silica (ethyl acetate/hexane, 35/65-70/30) to give ({4-[4-(3-trifluoromethylbenzyloxyimino)piperidine-1-sulfonyl]phenylcarbamoyl}methyl)carbamic acid tert-butyl ester (147 mg, 83%, colorless solid).

b) To a solution of ({4-[4-(3-trifluoromethylbenzyloxyimino)piperidine-1-sulfonyl]phenylcarbamoyl}methyl)carbamic acid tert-butyl ester (147 mg, 0.24 mmol) in ethyl acetate (2 ml), 4N—HCl in ethyl acetate (2 ml) was added at 0° C. and stirred at room temperature for 1.5 hours. The mixture was diluted with ether (30 ml) and precipitate was collected and dried to give the title compound (124 mg, 99%).

### Example 90

### 1-[4-(2-Aminoethylamino)benzenesulfonyl]piperidin-4-one O-(3-trifluoromethylbenzyl)oxime dihydrochloride

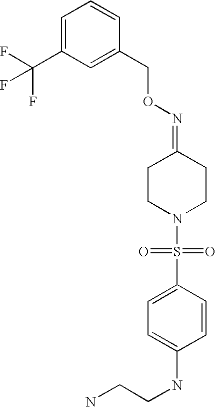

1-(4-Fluorobenzenesulfonyl)piperidin-4-one O-(3-trifluoromethylbenzyl)oxime (100 mg, 0.23 mmol) and ethylenediamine (70 mg, 1.16 mmol) were dissolved in toluene and stirred at 110° C. for 12 hours. The reaction mixture was diluted with 30 ml of ethyl acetate and washed with H₂O and brine. The organic layer was dried over MgSO₄ and concentrated. The residue was purified by column chromatography on silica (ethyl acetate/methanol, 100/0-90/10) to afford the desired compound as a free base (65 mg, 60%), which was dissolved in dioxane (2 ml) and treated with 4N—HCl in dioxane (2 ml) solution. The mixture was triturated with ether (10 ml) to give the title compound.

### Example 91

### 1-(4-Ethoxybenzenesulfonyl)piperidin-4-one O-(3-trifluoromethylbenzyl)oxime

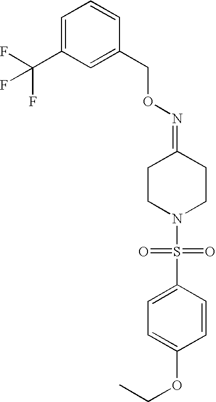

Under nitrogen atmosphere, sodium (27 mg, 1.16 mmol) was added to ethanol (1 ml) and stirred at room temperature for 1 hour. A solution of 1-(4-fluorobenzenesulfonyl)piperidin-4-one O-(3-trifluoromethylbenzyl)oxime (100 mg, 0.23 mmol) in tetrahydrofuran (1 ml) was added to this mixture and refluxed for 4 hours. The reaction mixture was concentrated under reduced pressure. Saturated aqueous NH4Cl solution (5 ml) was added, extracted with ethyl acetate (20 ml×2), washed with brine, dried (MgSO₄) and evaporated under reduced pressure. The residue was purified by column chromatography on silica (ethyl acetate/hexane, 20/80-40/60) to afford the title compound.

¹H-NMR (DMSO-d₆) δ: 7.72-7.52 (6H, m), 7.12 (2H, d), 5.04 (2H, s), 4.13 (2H, q), 3.08-2.92 (4H, m), 2.62 (2H, t), 2.32 (2H, t), 1.37 (3H, t).

### Example 92

### 1-(4-Propoxybenzenesulfonyl)piperidin-4-one O-(3-trifluoromethylbenzyl)oxime

was prepared as described in EXAMPLE 91

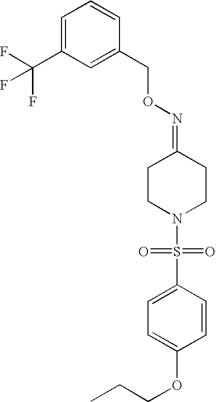

### Example 93

### 1-(4-Isopropoxybenzenesulfonyl)piperidin-4-one O-(3-trifluoromethylbenzyl)oxime

was prepared as described in EXAMPLE 91

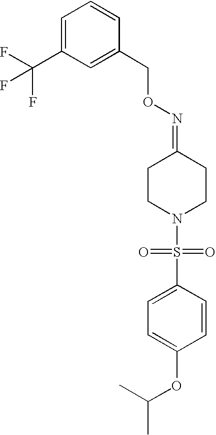

¹H-NMR (DMSO-d₆) δ: 7.70-7.56 (6H, m), 7.10 (2H, d), 5.04 (2H, s), 4.80-4.64 (1H, m), 3.04-2.92 (4H, m), 2.62 (2H, t), 2.32 (2H, t), 1.32 (6H, d).

### Example 94

### 1-[4-(2-Methoxyethoxy)benzenesulfonyl]piperidin-4-one O-(3-trifluoromethylbenzyl)oxime

was prepared as described in EXAMPLE 91

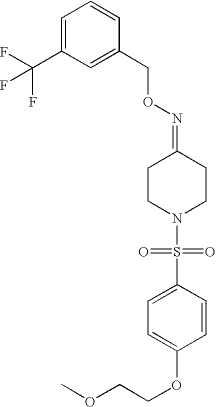

¹H-NMR (DMSO-d₆) δ: 7.72-7.51 (6H, m), 7.16 (2H, d), 5.07 (2H, s), 4.19 (2H, t), 3.68 (2H, t), 3.32 (3H, s), 3.10-2.94 (4H, m), 2.62 (2H, t), 2.32 (2H, t).

### Example 95

### 1-(4-Cyclopropylmethoxybenzenesulfonyl)piperidin-4-one O-(3-trifluoromethylbenzyl)oxime

was prepared as described in EXAMPLE 91

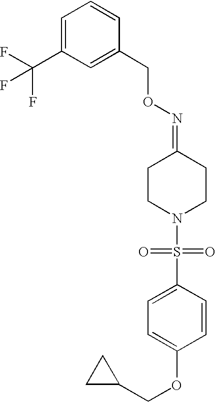

¹H-NMR (DMSO-d₆) δ: 7.72-7.50 (6H, m), 7.10 (2H, d), 5.03 (2H, s), 3.91 (2H, d), 3.04-2.92 (4H, m), 2.60 (2H, t), 2.30 (2H, t), 1.32-1.14 (1H, m), 0.62-0.54 (2H, m), 0.36-0.28 (2H, m).

### Example 96

### 1-(6-Dimethylaminopyridine-3-sulfonyl)piperidin-4-one O-(3-trifluoromethylbenzyl)oxime

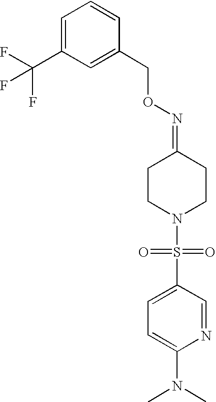

To a suspension of 1-(6-chloropyridine-3-sulfonyl)piperidin-4-one O-(3-trifluoromethylbenzyl)oxime (90 mg, 0.20 mmol) in methanol (1 ml), the 2.0 M solution of dimethylamine in methanol (0.300 ml, 0.60 mmol) and tetrahydrofuran (0.5 ml) were added and stirred at room temperature for 12 hours. The reaction mixture was concentrated under reduced pressure. The residue was diluted with ethyl acetate (50 ml), washed with H₂O and brine, dried (MgSO₄) and evaporated under reduced pressure. The residue was recrystallized from ethyl acetate/hexane to give the title compound (79 mg, 87%).

### Example 97

### 1-(6-Pyrrolidin-1-ylpyridine-3-sulfonyl)piperidin-4-one O-(3-trifluoromethylbenzyl)oxime

was prepared as described in EXAMPLE 96

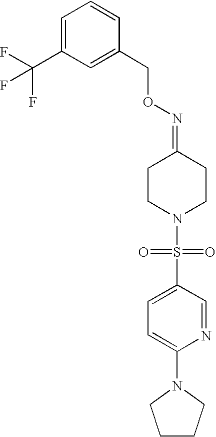

### Example 98

### 1-(6-Morpholin-4-ylpyridine-3-sulfonyl)piperidin-4-one O-(3-trifluoromethylbenzyl)oxime

was prepared as described in EXAMPLE 96

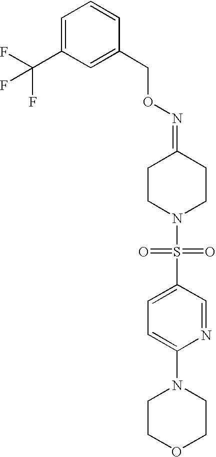

### Example 99

### 1-(6-Propoxypyridine-3-sulfonyl)piperidin-4-one O-(3-trifluoromethylbenzyl)oxime

was prepared as described in EXAMPLE 96

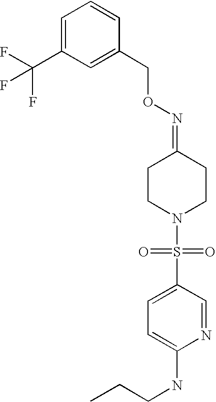

¹H-NMR (DMSO-d₆) δ: 8.23 (1H, s), 7.68-7.50 (6H, m), 6.56 (1H, d), 5.06 (2H, s), 3.30-3.20 (2H, m), 3.10-2.90 (4H, m), 2.65-2.57 (2H, m), 2.38-2.22 (2H, m), 1.56 (2H, dt), 0.90 (3H, t).

### Example 100

### 1-(6-Methoxypyridine-3-sulfonyl)piperidin-4-one O-(3-trifluoromethylbenzyl)oxime

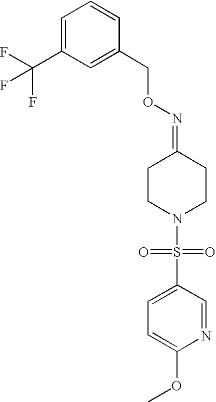

To a suspension of 1-(6-chloropyridine-3-sulfonyl)piperidin-4-one O-(3-trifluoromethylbenzyl)oxime (224 mg, 0.50 mmol) in methanol (5 ml), 1N—NaOMe in methanol (3.0 ml, 3.0 mmol) was added under nitrogen atmosphere. The mixture was refluxed for 3 hours. The reaction mixture was concentrated under reduced pressure. H2O (5 ml) was added, and the resulting precipitate was collected and dried to give the title compound (212 mg, 96%).

### Example 101

### 1-(6-Phenoxypyridine-3-sulfonyl)piperidin-4-one O-(3-trifluoromethylbenzyl)oxime

was prepared as described in EXAMPLE 100

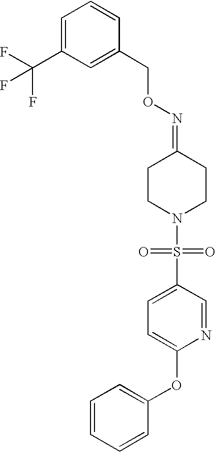

¹H-NMR (DMSO-d₆) δ: 8.51 (1H, s), 8.18 (1H, dd), 7.72-7.43 (6H, m), 7.32-7.18 (3H, m), 5.08 (2H, s), 3.18-3.04 (4H, m), 2.64 (2H, t), 2.37 (2H, t).

### Example 102

### 1-(6-Benzyloxypyridine-3-sulfonyl)piperidin-4-one O-(3-trifluoromethylbenzyl)oxime

was prepared as described in EXAMPLE 100

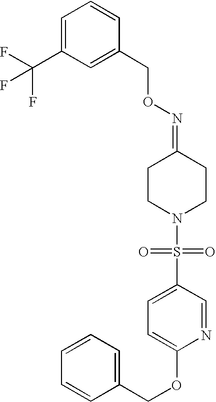

### Example 103

### 1-(6-Propoxypyridine-3-sulfonyl)piperidin-4-one O-(3-trifluoromethylbenzyl)oxime

was prepared as described in EXAMPLE 100

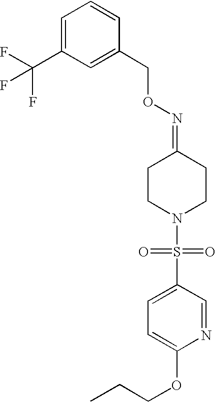

### Example 104

### 1-(6-Phenylpyridine-3-sulfonyl)piperidin-4-one O-(3-trifluoromethylbenzyl)oxime

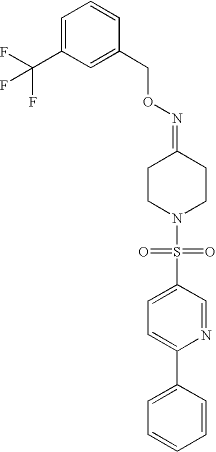

To a solution of 1-(6-chloropyridine-3-sulfonyl)piperidin-4-one O-(3-trifluoromethylbenzyl)oxime (90 mg, 0.20 mmol) and phenylboronic acid (29 mg, 0.24 mmol) in ethylene glycol dimethyl ether (0.400 ml) and ethanol (0.200 ml), tetrakis(triphenylphosphine)palladium (7 mg, 0.006 mmol) and 2N aqueous sodium carbonate solution (0.200 ml) were added under nitrogen atmosphere and refluxed for 5 hours. H₂O (5 ml) was added to the reaction mixture and extracted with ethyl acetate (20 ml×2), washed with brine, dried (MgSO₄) and evaporated under reduced pressure. The residue was purified by column chromatography on silica (ethyl acetate/hexane, 15/85-40/60) and recrystallized from ethyl acetate/hexane to give the title compound (175 mg, 77%).

### Example 105

### 5-[4-(3-Trifluoromethylbenzyloxyimino)piperidine-1-sulfonyl]-1H-pyridin-2-one

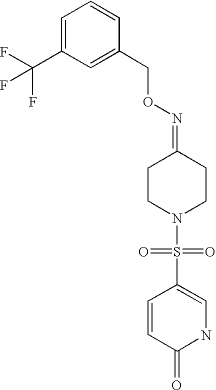

To a solution of 1-(6-methoxypyridine-3-sulfonyl)piperidin-4-one O-(3-trifluoromethylbenzyl)oxime (130 mg, 0.29 mmol) in acetonitrile (4 ml), chlorotrimethylsilane (160 mg, 1.47 mmol) in acetonitrile (2 ml) and KI (244 mg, 1.47 mmol) were added and stirred at 80° C. for 1 hour. The reaction mixture was concentrated under reduced pressure. The residue was diluted with ethyl acetate (50 ml), washed with H₂O, 10% aqueous Na₂S₂O₃ solution and brine, dried (MgSO₄) and evaporated under reduced pressure. The residue was recrystallized from methanol/H₂O to give the title compound (113 mg, 90%).

### Example 106

### 1-(4-Aminophenylmethanesulfonyl)piperidin-4-one O-(3-trifluoromethylbenzyl)oxime

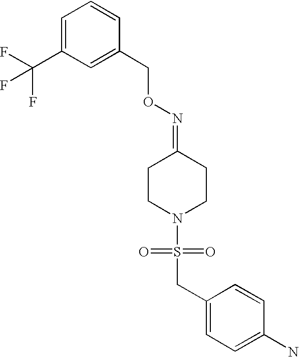

a) A solution of piperidin-4-one O-(3-trifluoromethylbenzyl)oxime (408 mg, 1.50 mmol) and triethylamine (0.460 ml, 3.30 mmol) in dichloromethane (4 ml), (4-nitrophenyl)methanesulfonyl chloride (390 mg, 1.65 mmol) in dichloromethane (2 ml) was slowly added at 0° C. and stirred at room temperature for 1 hour. Saturated aqueous NaHCO₃ solution (10 ml) was added, extracted with chloroform (30 ml×2), dried (MgSO₄) and evaporated under reduced pressure to give yellow solid. This crude material was taken up in toluene (13 ml). To the mixture, iron powder (420 mg, 7.50 mmol), NH₄Cl (802 mg, 15.0 mmol) and H₂O (1.5 ml) was added. The reaction mixture was heated at 105° C. and stirred for 2 hours. The reaction mixture was diluted with 30 ml of ethyl acetate, then filtrate through a pad of Celite. The filtrate was concentrated under reduced pressure. The residue was purified by column chromatography on silica (ethyl acetate/hexane, 30/70-60/40) to afford 1-(4-aminobenzenesulfonyl)piperidin-4-one O-(3-trifluoromethylbenzyl)oxime (471 mg, 95%, pale yellow oil). To a solution of the free base in ethyl acetate, 4N—HCl in ethyl acetate was added. The resulting precipitate was collected and dried in vacuo to give the title compound (417 mg, 63%).

### Example 107

### 1-(4-Trifluoromethoxybenzenesulfonyl)piperidin-4-one O-pyridin-2-ylmethyloxime

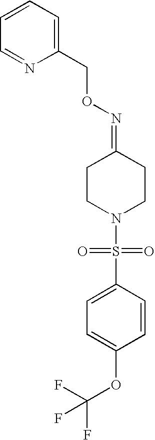

a) 4-(Trifluoromethoxy)benzenesulfonyl chloride (4.95 g, 18.6 mmol) was added to a solution of 4-piperidone monohydrate hydrochloride (2.92 g, 18.6 mmol) and N,N-diisopropylethylamine (9.60 ml, 55.8 mmol) in N,N-dimethylformamide (60 ml) at 0° C. and stirred at room temperature for 30 minutes and at 80° C. for 30 minutes. After cooling to room temperature, the reaction mixture was diluted with ethyl acetate (150 ml), washed with H₂O (50 ml) and brine, dried (MgSO₄) and evaporated under reduced pressure. The residue was purified by column chromatography on silica (ethyl acetate/hexane 30/70-50/50) to give 1-(4-(trifluoromethoxy)phenylsulfonyl)piperidin-4-one as a white solid (3.38 g, 53%, colorless solid).

b) A solution of 1-(4-(trifluoromethoxy)phenylsulfonyl)piperidin-4-one (102 mg, 0.300 mmol), O-pyridin-2-ylmethylhydroxylamine dihydrochloride (59 mg, 0.30 mmol) and sodium acetate (49 mg, 0.60 mmol) in ethanol (3 ml) was stirred at room temperature for 30 minutes. The reaction mixture was concentrated under reduced pressure and the residue was diluted with ethyl acetate (30 ml), washed with saturated aqueous NaHCO₃ solution and brine, dried (MgSO₄) and evaporated under reduced pressure. The residue was purified by column chromatography on silica (ethyl acetate/hexane 30/70-80/20) to give the title compound (117 mg, 91%).

### Example 108

### 2-Fluoro-5-[1-(4-trifluoromethoxybenzenesulfonyl)piperidin-4-ylideneaminooxymethyl]benzonitrile

was prepared as described in EXAMPLE 107. Starting material was 5-(aminooxymethyl)-2-fluorobenzonitrile (see example A3).

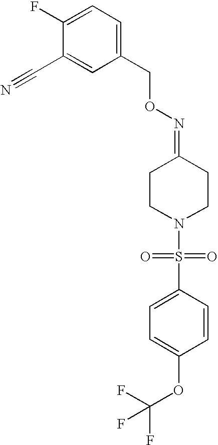

¹H-NMR (DMSO-d₆) δ: 7.90 (2H, d), 7.84 (1H, dd), 7.71-7.70 (1H, m), 7.62 (2H, d), 7.48 (1H, t), 5.00 (2H, s), 3.13-3.10 (4H, m), 2.63 (2H, t), 2.34 (2H, t).

### Example 109

### 1-(4-Trifluoromethoxybenzenesulfonyl)piperidin-4-one O-(4-chlorobenzyl)oxime

was prepared as described in EXAMPLE 107

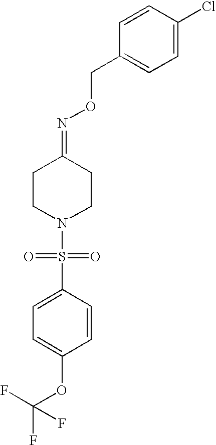

### Example 110

### 1-(4-Trifluoromethoxybenzenesulfonyl)piperidin-4-one O-(6-methoxypyridin-2-ylmethyl)oxime

was prepared as described in EXAMPLE 107. Starting material was O-((6-methoxypyridin-2-yl)methyl)hydroxylamine (see example A4).

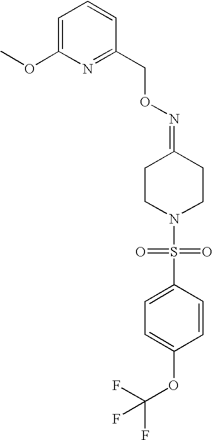

¹H-NMR (DMSO-d₆) δ: 7.91 (2H, d), 7.66-7.62 (3H, m), 6.87 (1H, d), 6.68 (1H, d), 4.96 (2H, s), 3.79 (3H, s), 3.14-3.13 (4H, m), 2.68 (2H, t), 2.35 (2H, t).

### Example 111

### 1-(4-Trifluoromethoxybenzenesulfonyl)piperidin-4-one O-(2-methoxypyridin-3-ylmethyl)oxime

was prepared as described in EXAMPLE 107. Starting material was O-((2-methoxypyridin-3-yl)methyl)hydroxylamine (see example A5).

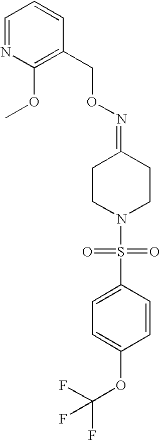

¹H-NMR (DMSO-d₆) δ: 8.08 (1H, dd), 7.91 (2H, d), 7.63 (3H, d), 7.58 (3H, dd), 6.94 (1H, dd), 4.94 (2H, s), 3.85 (3H, s), 3.13-3.10 (4H, m), 2.63 (2H, t), 2.34 (2H, t).

### Example 112

### 3-[1-(4-Trifluoromethoxybenzenesulfonyl)piperidin-4-ylideneaminooxymethyl]-1H-pyridin-2-one

was prepared as described in EXAMPLE 145

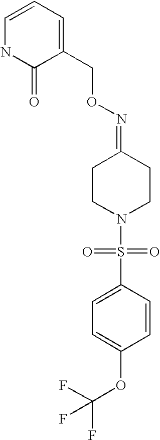

### Example 113

### 6-[1-(4-Trifluoromethoxy-benzenesulfonyl)-piperidin-4-ylideneaminooxymethyl]-1H-pyridin-2-one

was prepared as described in EXAMPLE 145

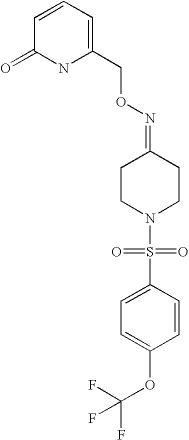

### Example 114

### 1-(4-Trifluoromethoxybenzenesulfonyl)piperidin-4-one O-(tetrahydropyran-2-ylmethyl)oxime

was prepared as described in EXAMPLE 107

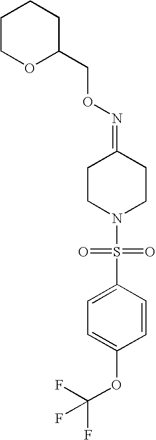

### Example 115

### 1-(4-Trifluoromethoxybenzenesulfonyl)piperidin-4-one O-cyclohexylmethyloxime

was prepared as described in EXAMPLE 107

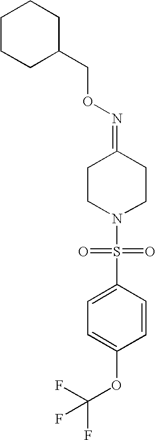

### Example 116

### 1-(4-Trifluoromethoxybenzenesulfonyl)piperidin-4-one O-cyclopentylmethyloxime

was prepared as described in EXAMPLE 107

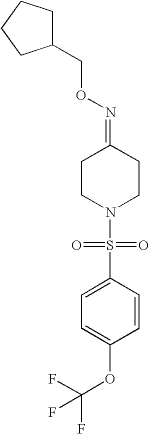

### Example 117

### 1-(4-Trifluoromethoxybenzenesulfonyl)piperidin-4-one O-pyrazin-2-ylmethyloxime

was prepared as described in EXAMPLE 107

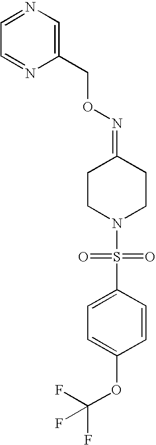

### Example 118

### 1-(4-Trifluoromethoxybenzenesulfonyl)piperidin-4-one O-(4-chloropyridin-2-ylmethyl)oxime

was prepared as described in EXAMPLE 107

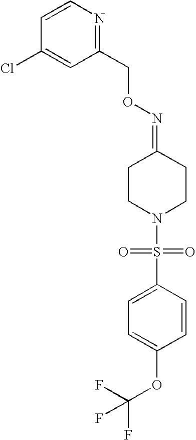

¹H-NMR (DMSO-d₆) δ: 8.48 (1H, d), 7.91 (2H, d), 7.64 (2H, d), 7.43 (1H, dd), 7.33 (1H, d), 5.06 (2H, s), 3.12-3.11 (4H, m), 2.70 (2H, t), 2.35 (2H, t).

### Example 119

### 1-(4-Trifluoromethoxybenzenesulfonyl)piperidin-4-one O-[bis(4-fluorophenyl)methyl]oxime

was prepared as described in EXAMPLE 107

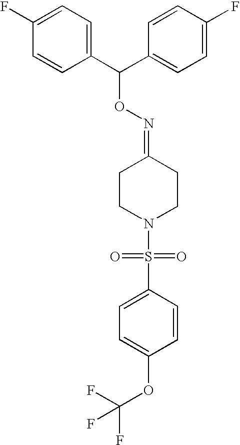

¹H-NMR (DMSO-d₆) δ: 7.90 (2H, d), 7.63 (2H, d), 7.34-7.32 (4H, m), 7.14-7.11 (4H, m), 6.15 (1H, s), 3.13-3.07 (4H, m), 2.73 (2H, t), 2.30 (2H, t).

### Example 120

### 2-[1-(4-Trifluoromethoxybenzenesulfonyl)piperidin-4-ylideneaminooxymethyl]benzonitrile

was prepared as described in EXAMPLE 107

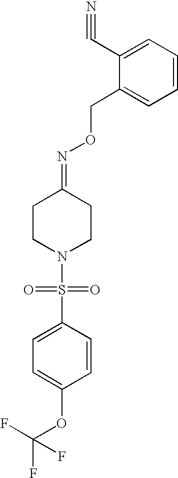

¹H-NMR (DMSO-d₆) δ: 7.90 (2H, d), 7.82 (1H, d), 7.64 (3H, dd), 7.52 (2H, dd), 5.14 (2H, s), 3.14-3.08 (4H, m), 2.63 (2H, t), 2.33 (2H, t).

### Example 121

### 3-[1-(4-Trifluoromethoxybenzenesulfonyl)piperidin-4-ylideneaminooxymethyl]benzonitrile

was prepared as described in EXAMPLE 107

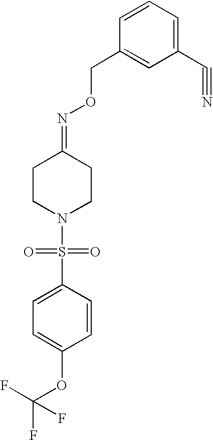

¹H-NMR (DMSO-d₆) δ: 7.90 (2H, d), 7.74 (2H, d), 7.62 (3H, d), 7.54 (1H, t), 5.03 (2H, s), 3.15-3.08 (4H, m), 2.64 (2H, t), 2.34 (2H, t).

### Example 122

### 4-[1-(4-Trifluoromethoxybenzenesulfonyl)piperidin-4-ylideneaminooxymethyl]benzonitrile

was prepared as described in EXAMPLE 107

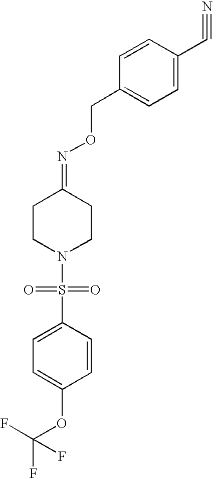

### Example 123

### 1-(4-Trifluoromethoxybenzenesulfonyl)piperidin-4-one O-(4-fluorobenzyl)oxime

was prepared as described in EXAMPLE 107

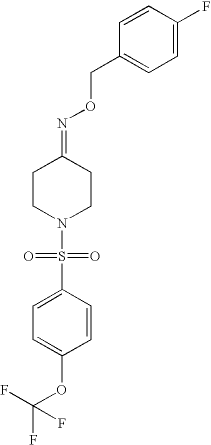

### Example 124

### 1-(4-Trifluoromethoxybenzenesulfonyl)piperidin-4-one O-(2-aminobenzyl)oxime

was prepared as described in EXAMPLE 107

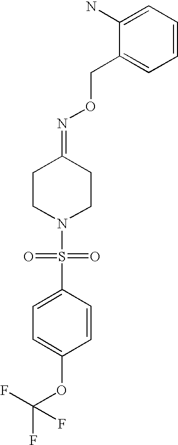

### Example 125

### 1-(4-Trifluoromethoxybenzenesulfonyl)piperidin-4-one O-(5-fluoropyridin-2-ylmethyl)oxime

was prepared as described in EXAMPLE 107. Starting material was O-((5-fluoropyridin-2-yl)methyl)hydroxylamine (see example A6).

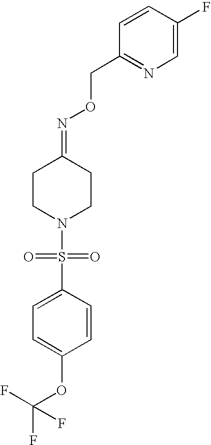

### Example 126

### 1-(4-Trifluoromethoxybenzenesulfonyl)piperidin-4-one O-(3-fluoropyridin-2-ylmethyl)oxime

was prepared as described in EXAMPLE 107. Starting material was O-((3-fluoropyridin-2-yl)methyl)hydroxylamine (see example A7).

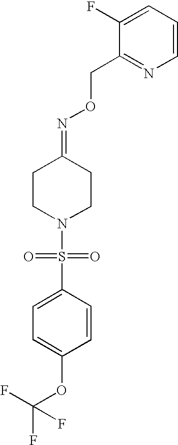

### Example 127

### 1-(4-Trifluoromethoxybenzenesulfonyl)piperidin-4-one O-(5-chloropyridin-2-ylmethyl)oxime

was prepared as described in EXAMPLE 107. Starting material was O-((5-chloropyridin-2-yl)methyl)hydroxylamine (see example A8).

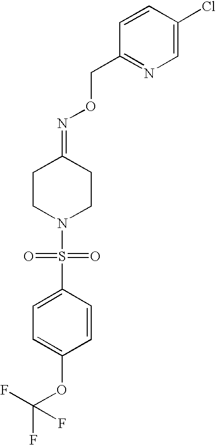

¹H-NMR (DMSO-d₆) δ: 8.55 (1H, d), 7.91-7.87 (3H, m), 7.63 (2H, d), 7.35 (1H, d), 5.05 (2H, s), 3.14 (4H, q), 2.67 (2H, t), 2.34 (2H, t).

### Example 128

### 1-(4-Trifluoromethoxybenzenesulfonyl)piperidin-4-one O-(3-dimethylaminobenzyl)oxime

was prepared as described in EXAMPLE 107. Starting material was 3-(aminooxymethyl)-N,N-dimethylaniline (see example A9).

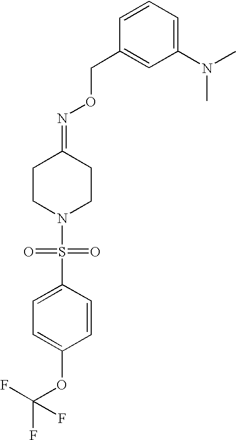

¹H-NMR (DMSO-d₆) δ: 7.91-7.88 (2H, m), 7.63 (2H, d), 7.12-7.08 (1H, m), 6.62-6.55 (3H, m), 4.91 (2H, s), 3.10 (4H, dt), 2.61 (2H, t), 2.34 (2H, t).

### Example 129

### 1-(4-Trifluoromethoxybenzenesulfonyl)piperidin-4-one O-[1-(4-chlorophenyl)ethyl]oxime

was prepared as described in EXAMPLE 107

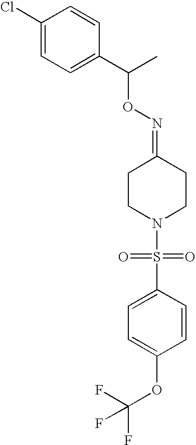

### Example 130

### 1-(4-Trifluoromethoxybenzenesulfonyl)piperidin-4-one O-piperidin-3-ylmethyloxime

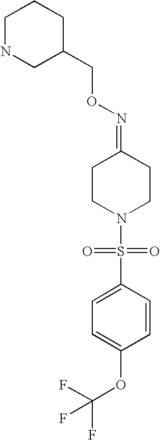

3-[1-(4-Trifluoromethoxybenzenesulfonyl)piperidin-4-ylideneaminooxymethyl]piperidine-1-carboxylic acid tert-butyl ester (prepared as described in Example 107, 389 mg, 0.90 mmol) was dissolve in dichloroethane (5 ml) and cooled to 0° C. The mixture was added trifluoroacetic acid (1 ml) and stirred at 0° C. for 30 minutes. The reaction mixture was concentrated under reduced pressure and the residue was diluted with ethyl acetate (60 ml), washed with saturated aqueous NaHCO₃ solution (20 ml) and brine, dried (MgSO₄) and evaporated under reduced pressure. The residue was purified by column chromatography on silica (ethyl acetate/methanol 100/0-80/20) to give the title compound (216 mg, 55%).

### Example 131

### 1-(4-Trifluoromethoxybenzenesulfonyl)piperidin-4-one O-(1-methylpiperidin-3-ylmethyl)oxime

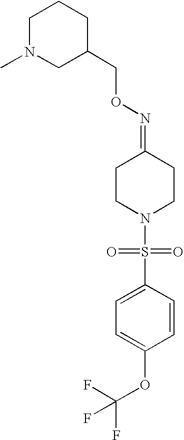

To a solution of 1-(4-trifluoromethoxybenzenesulfonyl)piperidin-4-one O-piperidin-3-ylmethyloxime (60 mg, 0.14 mmol) in tetrahydrofuran (3 ml), formalin (0.014 ml, 0.166 mmol) and sodium triacetoxyborohydride (75 mg, 0.35 mmol) were added and stirred at room temperature for 1 hour. The reaction was quenched with saturated aqueous NaHCO₃ solution (5 ml), extracted with chloroform (30 ml×2), dried (MgSO4) and evaporated under reduced pressure. The residue was purified by column chromatography on silica (ethyl acetate/hexane 50/50-80/20) to give the title compound (59 mg, 95%).

### Example 132

### 1-(4-Trifluoromethoxybenzenesulfonyl)piperidin-4-one O-(1-acetylpiperidin-3-ylmethyl)oxime

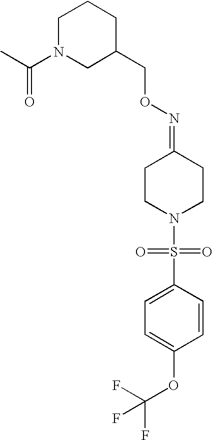

To a solution of 1-(4-trifluoromethoxybenzenesulfonyl)piperidin-4-one O-piperidin-3-ylmethyloxime (60 mg, 0.14 mmol) and triethylamine (0.023 ml, 0.17 mmol) in dichloroethane (2 ml), acetyl chloride (13 mg, 0.17 mmol) was added at 0° C. and stirred at 0° C. for 30 minutes. The reaction was quenched with saturated aqueous NaHCO₃ solution (5 ml), extracted with chloroform (20 ml×2), dried (MgSO4) and evaporated under reduced pressure. The residue was purified by column chromatography on silica (ethyl acetate/hexane 40/60-60/40) to give the title compound (61 mg, 93%).

### Example 133

### 1-(4-Trifluoromethoxybenzenesulfonyl)piperidin-4-one O-(3-aminobenzyl)oxime

was prepared as described in EXAMPLE 107

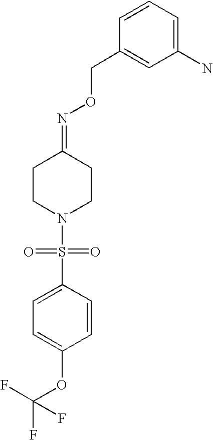

### Example 134

### 1-(4-Trifluoromethoxybenzenesulfonyl)piperidin-4-one O-(4-nitrobenzyl)oxime

was prepared as described in EXAMPLE 107.

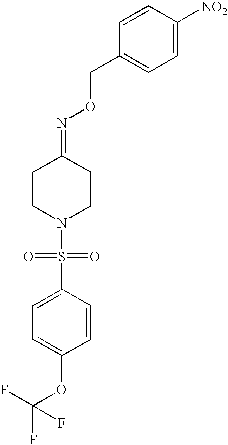

¹H-NMR (DMSO-d₆) δ: 8.18 (2H, d), 7.91 (2H, d), 7.63 (2H, d), 7.55 (2H, d), 5.13 (2H, s), 3.14 (4H, q), 2.66 (2H, t), 2.34 (2H, t).

### Example 135

### 1-(4-Trifluoromethoxybenzenesulfonyl)piperidin-4-one O-(4-aminobenzyl)oxime

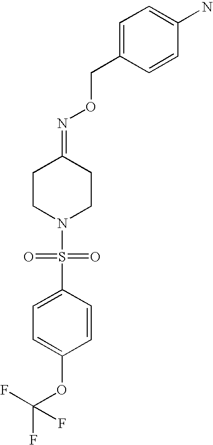

A suspension of 1-(4-trifluoromethoxybenzenesulfonyl)piperidin-4-one O-(4-nitrobenzyl)oxime (200 mg, 0.42 mmol), iron powder (118 mg, 2.11 mmol) and NH4Cl (225 mg, 4.2 mmol) in toluene (4 ml) and H₂O (0.3 ml) was stirred at 90° C. overnight. The reaction mixture was filtrated through a pad of Celite and the residue was washed with ethyl acetate/methanol (4:1). The filtrate was concentrated under reduced pressure and the residue was diluted with chloroform, washed with H₂O, dried (MgSO₄) and evaporated under reduced pressure. The residue was purified by column chromatography on silica (ethyl acetate/hexane 10/90-30/70) to give the title compound (176 mg, 95%).

### Example 136

### 1-(4-Trifluoromethoxybenzenesulfonyl)piperidin-4-one O-piperidin-4-ylmethyloxime

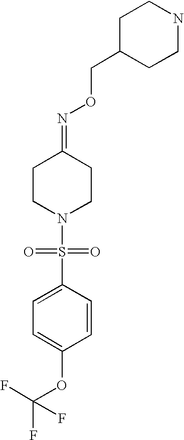

4-[1-(4-Trifluoromethoxybenzenesulfonyl)piperidin-4-ylideneaminooxymethyl]piperidine-1-carboxylic acid tert-butyl ester (prepared as described in Example 107, 273 mg, 0.51 mmol) was dissolve in dichloroethane (4 ml) and cooled to 0° C. The mixture was added trifluoroacetic acid (0.38 ml, 5.1 mmol) and stirred at room temperature for 1 hour. The reaction mixture was concentrated under reduced pressure and the residue was diluted with chloroform, washed with saturated aqueous NaHCO₃ solution (20 ml) and H₂O, dried (MgSO₄) and evaporated under reduced pressure. The residue was purified by column chromatography on silica (ethyl acetate/methanol 97/3-70/30) to give the title compound (196 mg, 90%).

### Example 137

### 1-(4-Trifluoromethoxybenzenesulfonyl)piperidin-4-one O-(4-aminobenzyl)oxime

was prepared as described in Example 131.

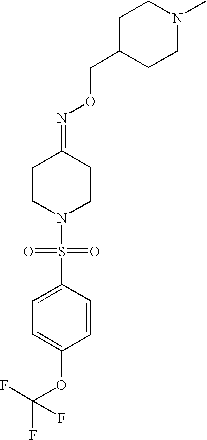

### Example 138

### 1-(4-Trifluoromethoxybenzenesulfonyl)piperidin-4-one O-(1-acetylpiperidin-4-ylmethyl)oxime

was prepared as described in Example 132.

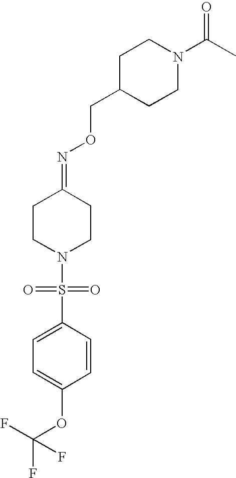

### Example 139

### 1-(4-Trifluoromethoxybenzenesulfonyl)piperidin-4-one O-(tetrahydropyran-4-ylmethyl)oxime

was prepared as described in Example 107.

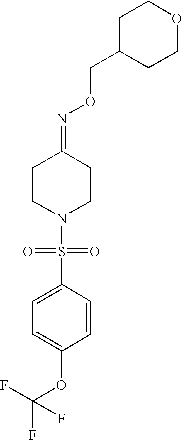

### Example 140

### 1-Methyl-6-[1-(4-trifluoromethoxybenzenesulfonyl)piperidin-4-ylideneaminooxymethyl]-1H-pyridin-2-one

A suspension of 6-[1-(4-trifluoromethoxy-benzenesulfonyl)-piperidin-4-ylideneaminooxymethyl]-1H-pyridin-2-one (prepared as described in EXAMPLE 113, 60 mg, 0.13 mmol), K₂CO₃ (47 mg, 0.34 mmol) and iodomethane (0.50 ml) in acetone was refluxed for 16 hours. The reaction mixture was concentrated under reduced pressure and the residue was diluted with chloroform (60 ml), washed with H₂O (10 ml), dried (MgSO₄) and evaporated under reduced pressure. The residue was purified by column chromatography on silica (ethyl acetate/methanol 100/0-90/10) to give the title compound (79 mg, 100%).

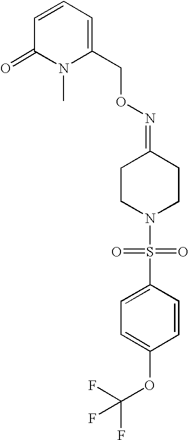

### Example 141

### 1-Methyl-3-[1-(4-trifluoromethoxybenzenesulfonyl)piperidin-4-ylideneaminooxymethyl]-1H-pyridin-2-one

was prepared as described in Example 140.

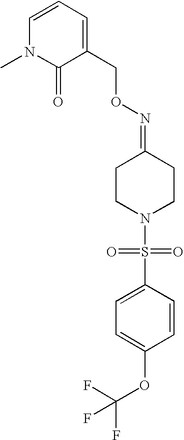

### Example 142

### 1-(4-Trifluoromethoxybenzenesulfonyl)piperidin-4-one O-(3-methyl-3H-imidazol-4-ylmethyl)oxime

was prepared as described in Example 107.

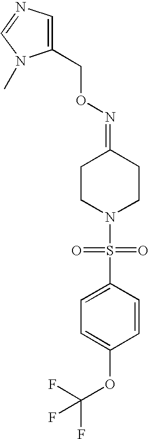

### Example 143

### 1-(4-Trifluoromethoxybenzenesulfonyl)piperidin-4-one O-(5-chlorothiophen-2-ylmethyl)oxime

was prepared as described in Example 107.

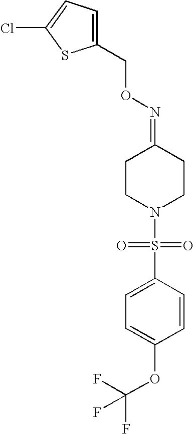

### Example 144

### 1-(4-Trifluoromethoxybenzenesulfonyl)piperidin-4-one O-(6-methoxypyridin-3-ylmethyl)oxime

was prepared as described in Example 107. Starting material was O-((6-methoxypyridin-3-yl)methyl)hydroxylamine (see example A10).

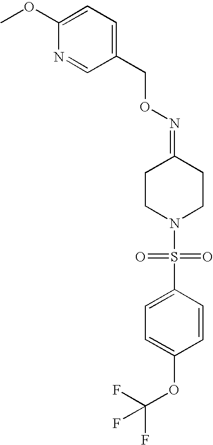

¹H-NMR (DMSO-d₆) δ: 8.11 (1H, s), 7.90 (2H, d), 7.65-7.61 (3H, m), 6.77 (1H, d), 4.92 (2H, s), 3.83 (3H, s), 3.14-3.07 (4H, m), 2.57 (2H, t), 2.34 (2H, t).

### Example 145

### 5-[1-(4-Trifluoromethoxybenzenesulfonyl)piperidin-4-ylideneaminooxymethyl]-1H-pyridin-2-one

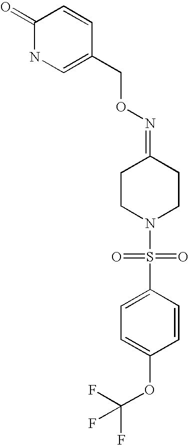

To a solution of 1-(4-trifluoromethoxybenzenesulfonyl)piperidin-4-one O-(6-methoxypyridin-3-ylmethyl)oxime (492 mg, 1.07 mmol) in acetonitrile (8 ml), chlorotrimethylsilane (350 mg, 3.21 mmol) in acetonitrile (2 ml) and KI (533 mg, 3.21 mmol) were added and refluxed for 2 hour. The reaction mixture was diluted with chloroform (50 ml), washed with 10% aqueous Na₂S₂O₃ solution, dried (MgSO₄) and evaporated under reduced pressure. The residue was purified by column chromatography on silica (chloroform/methanol 100/0-90/10) to give the title compound (421 mg, 89%).

### Example 146

### 1-Methyl-5-[1-4-trifluoromethoxy-benzenesulfonyl)-piperidin-4-ylideneaminooxymethyl]-1H-pyridin-2-one

was prepared as described in Example 140.

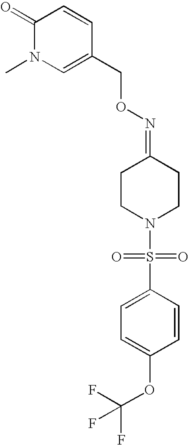

### Example 147

### 1-(4-Trifluoromethoxybenzenesulfonyl)piperidin-4-one O-(2-chloro-6-methoxypyridin-4-ylmethyl)oxime

was prepared as described in Example 107. Starting material was O-((2-chloro-6-methoxypyridin-4-yl)methyl)hydroxylamine (see example A11).

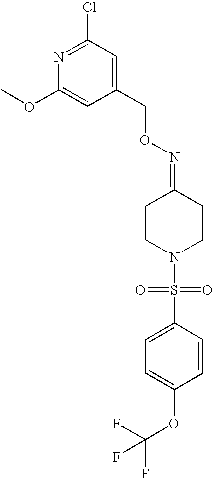

¹H-NMR (DMSO-d₆) δ: 7.91 (2H, d), 7.63 (2H, d), 6.94 (1H, s), 6.68 (1H, s), 4.99 (2H, s), 3.82 (3H, s), 3.14-3.12 (4H, m), 2.67 (2H, t), 2.34 (2H, t).

### Example 148

### 1-(4-Trifluoromethoxybenzenesulfonyl)piperidin-4-one O-(2-methoxypyridin-4-ylmethyl)oxime

was prepared as described in Example 107. Starting material was O-((2-methoxypyridin-4-yl)methyl)hydroxylamine (see example A12).

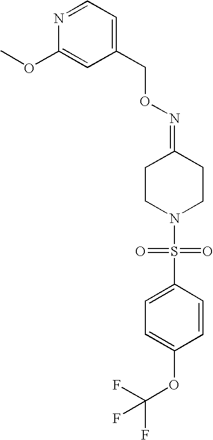

### Example 149

### 6-Chloro-4-[1-(4-trifluoromethoxybenzenesulfonyl)piperidin-4-ylideneaminooxymethyl]-1H-pyridin-2-one

was prepared as described in Example 145.

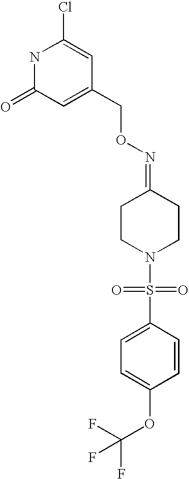

### Example 150

### 4-[1-(4-Trifluoromethoxybenzenesulfonyl)piperidin-4-ylideneaminooxymethyl]-1H-pyridin-2-one

was prepared as described in Example 145.

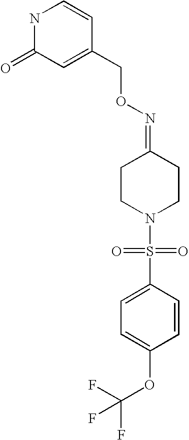

### Example 151

### 1-Methyl-4-[1-(4-trifluoromethoxybenzenesulfonyl)piperidin-4-ylideneaminooxymethyl]-1H-pyridin-2-one

was prepared as described in Example 140.

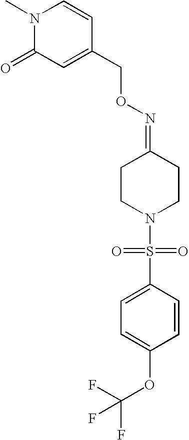

### Example 152

### 6-Chloro-1-methyl-4-[1-(4-trifluoromethoxybenzenesulfonyl)piperidin-4-ylideneaminooxymethyl]-1H-pyridin-2-one

was prepared as described in Example 140.

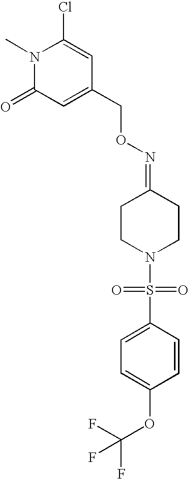

¹H-NMR (DMSO-d₆) δ: 7.91 (2H, d), 7.63 (2H, d), 6.35 (1H, s), 6.25 (1H, s), 4.82 (2H, s), 3.50 (3H, s), 3.14-3.12 (4H, m), 2.67 (2H, t), 2.35 (2H, t).

### Example 153

### 2-Fluoro-5-{1-[2-(4-fluorophenyl)acetyl]piperidin-4-ylideneaminooxymethyl}benzonitrile

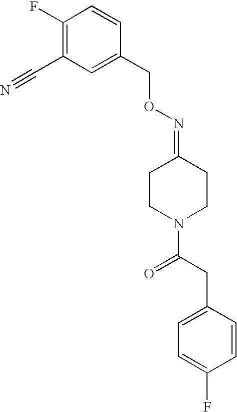

a) The mixture of 5-(Aminooxymethyl)-2-fluorobenzonitrile hydrochloride (1.74 g, 8.57 mmol), 4-piperidone monohydrate hydrochloride (1.35 g, 8.57 mmol) and sodium acetate (1.41 g, 17.2 mmol) in ethanol (60 ml) was stirred at room temperature for 6 hours. The solvent was evaporated under reduced pressure. The residue was diluted with chloroform (100 ml), washed with saturated aqueous NaHCO₃ solution (50 ml), dried (MgSO₄) and evaporated under reduced pressure to give 2-fluoro-5-((piperidin-4-ylideneaminooxy)methyl)benzonitrile (2.19 g, 100%, pale yellow oil).

b) To a solution of 2-fluoro-5-((piperidin-4-ylideneaminooxy)methyl)benzonitrile (99 mg, 0.40 mmol), 4-fluorophenylacetic acid (68 mg, 0.44 mmol) and 1-hydroxybenzotriazole (54 mg, 0.40 mmol) in dichloromethane (4 ml), N-ethyldimethylaminopropylcarbodiimide hydrochloride (92 mg, 0.48 mmol) was added and stirred at room temperature for 2 hours. The reaction was quenched with saturated aqueous NaHCO₃ solution (10 ml) and extracted with chloroform (20 ml×2). The organic layer was dried (MgSO₄) and evaporated under reduced pressure. The residue was purified by column chromatography on silica (ethyl acetate/hexane, 50/50-100/0) to give the title compound (131 mg, 85%).

### Example 154

### 2-Fluoro-5-{1-[3-(4-fluorophenyl)propionyl]piperidin-4-ylideneaminooxymethyl}benzonitrile

was prepared as described in Example 153.

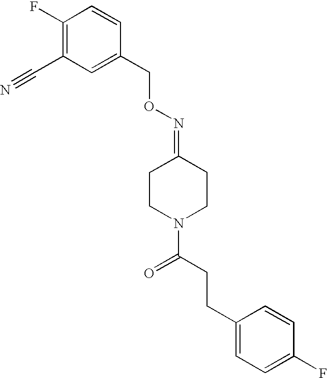

### Example 155

### 2-Fluoro-5-{1-[(E)-3-(4-fluorophenyl)acryloyl]piperidin-4-ylideneaminooxymethyl}benzonitrile

was prepared as described in Example 153.

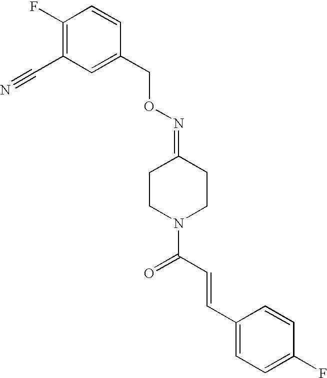

### Example 156

### 2-Fluoro-5-{1-[2-(4-fluorophenoxy)acetyl]piperidin-4-ylideneaminooxymethyl}benzonitrile

was prepared as described in Example 153.

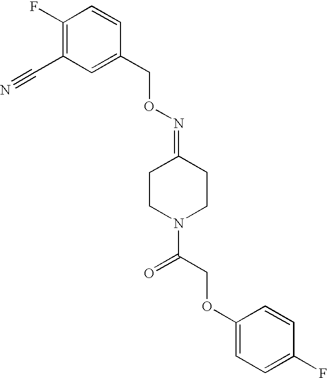

### Example 157

### 4-(3-Cyano-4-fluorobenzyloxyimino)piperidine-1-carboxylic acid 4-fluorobenzylamide

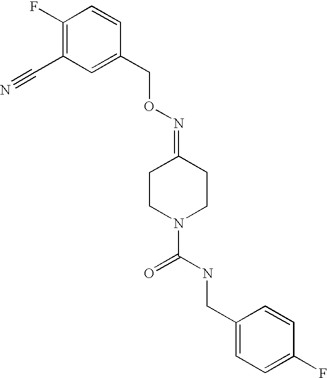

To a solution of 2-fluoro-5-((piperidin-4-ylideneaminooxy)methyl)benzonitrile (99 mg, 0.40 mmol) in acetonitrile (4 ml), 4-fluorophenyl isocyanate (67 mg, 0.44 mmol) was added and stirred at room temperature for 1 hour. The reaction was quenched with methanol (10 ml) and the solvent was evaporated under reduced pressure. The residue was purified by column chromatography on silica (ethyl acetate/hexane, 45/55-75/25) to give the title compound (90 mg, 57%).

### Example 158

### 4-(3-Cyano-4-fluorobenzyloxyimino)piperidine-1-carboxylic acid 4-fluorophenyl ester

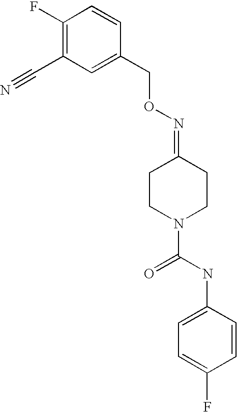

To a solution of 2-fluoro-5-((piperidin-4-ylideneaminooxy)methyl)benzonitrile (99 mg, 0.40 mmol) and triethylamine (0.061 ml, 0.44 mmol) in dichloromethane (4 ml), 4-fluorophenyl chloroformate (78 mg, 0.44 mmol) was added at 0° C. and stirred at 0° C. for 1 hour. The reaction was quenched with saturated aqueous NaHCO₃ solution (10 ml) and extracted with chloroform (20 ml×2). The organic layer was dried (MgSO₄) and evaporated under reduced pressure. The residue was purified by column chromatography on silica (ethyl acetate/hexane, 15/85-60/40) to give the title compound (150 mg, 97%).

### Example 159

### 5-[1-(2-Aminobenzoyl)piperidin-4-ylideneaminooxymethyl]-2-fluorobenzonitrile

was prepared as described in Example 153.

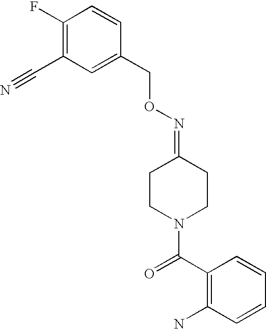

### Example 160

### 1-(4-(Trifluoromethoxy)phenylsulfonyl)piperidin-4-one O-(1-methyl-1H-benzo[d]imidazol-2-yl)methyl oxime

was prepared as described in Example 107.

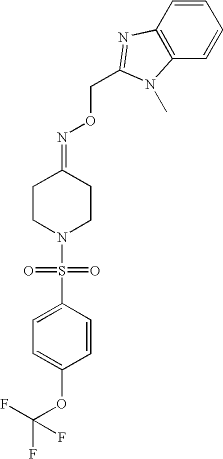

¹H-NMR (DMSO-d₆) δ: 7.89 (2H, d), 7.60-7.54 (4H, m), 7.26-7.19 (2H, m), 5.25 (2H, s), 3.78 (3H, s), 3.15-3.08 (4H, m), 2.61 (2H, t), 2.33 (2H, t).

### Example 161

### 1-(4-(Trifluoromethoxy)phenylsulfonyl)piperidin-4-one O-(1-methyl-1H-imidazol-2-yl)methyl oxime

was prepared as described in Example 107.

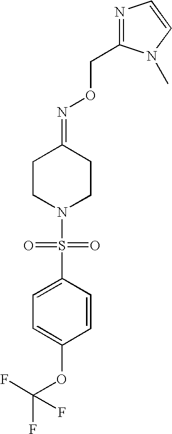

### Example 162

### 1-(4-(Trifluoromethoxy)phenylsulfonyl)piperidin-4-one O-(1H-benzo[d]imidazol-2-yl)methyl oxime

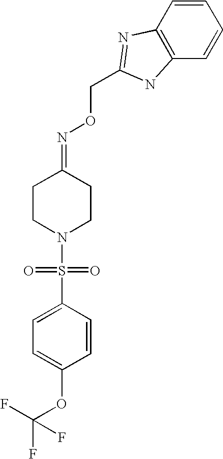

To a solution of tert-Butyl 2-((1-(4-(trifluoromethoxy)phenylsulfonyl)piperidin-4-ylideneaminooxy)methyl)-1H-benzo[d]imidazole-1-carboxylate (prepared as described in Example 107, 275 mg, 0.484 mmol) in dichloromethane (8 ml), trifluoroacetic acid (4 ml) was added at 0° C. and stirred at room temperature for 2 hours. The solvent was removed under reduced pressure. Saturated aqueous NaHCO₃ solution (10 ml) was added and extracted with chloroform. The organic layer was washed with H₂O, dried (Na₂SO₄) and evaporated under reduced pressure. The residual solid was triturated with ethyl acetate/methanol (90/10) to give the title compound (160 mg, 70%).

¹H-NMR (DMSO-d₆) δ: 12.32 (1H, s), 7.90 (2H, t), 7.62 (2H, d), 7.48 (2H, br), 7.16 (2H, d), 5.15 (2H, s), 3.12 (4H, q), 2.67 (2H, t), 2.35 (2H, t).

### Example 163

### 5-((1-(3,3-Bis(4-fluorophenyl)acryloyl)piperidin-4-ylideneaminooxy)methyl)-2-fluorobenzonitrile

was prepared as described in Example 153.

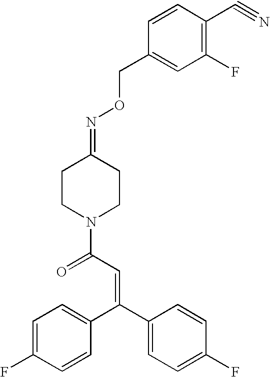

¹H-NMR (DMSO-d₆) δ: 7.86 (1H, t), 7.73 (1H, d), 7.52 (1H, t), 7.33 (2H, t), 7.22 (6H, m), 6.56 (1H, s), 5.02 (2H, s), 3.45 (4H, t), 2.41 (1H, t), 2.19 (2H, dt), 1.98 (1H, t).

### Example 164

### 5-((1-(3,3-Bis(4-fluorophenyl)propanoyl)piperidin-4-ylideneaminooxy)methyl)-2-fluorobenzonitrile

was prepared as described in Example 153.

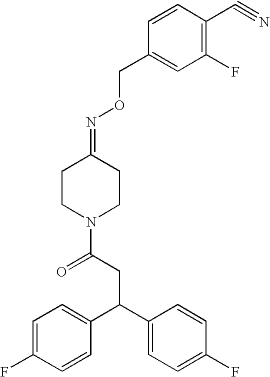

¹H-NMR (DMSO-d₆) δ: 7.87 (1H, d), 7.75 (1H, t), 7.52 (1H, t), 7.35 (4H, dd), 7.08 (4H, t), 5.03 (2H, s), 4.53 (1H, t), 3.63-3.57 (2H, m), 3.45 (2H, q), 3.15 (2H, t), 2.47-2.39 (2H, m), 2.19 (2H, dt).

### Example 165

### 5-((1-(6-(Cyclopropylamino)pyridin-3-ylsulfonyl)piperidin-4-ylideneaminooxy)methyl)-2-fluorobenzonitrile

was prepared as described in EXAMPLE 96

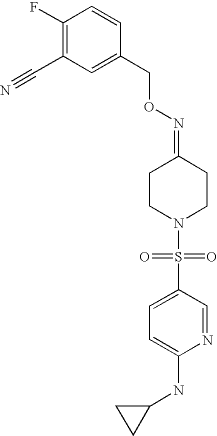

### Example 166

### 2-Fluoro-5-((1-(2-(4-fluorophenylamino)acetyl)piperidin-4-ylideneaminooxy)methyl)benzonitrile

was prepared as described in Example 153.

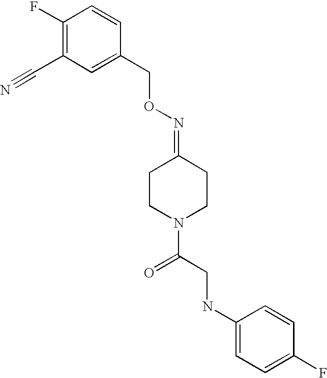

### Example 167

### 2-(4-(3-Cyano-4-fluorobenzyloxyimino)piperidin-1-yl)-N-(4-fluorophenyl)acetamide

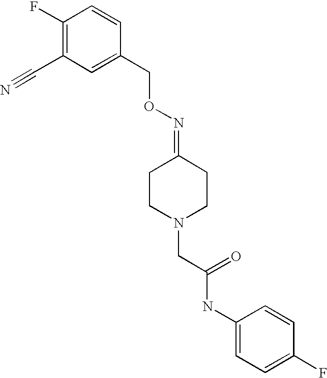

The mixture of 2-fluoro-5-((piperidin-4-ylideneaminooxy)methyl)benzonitrile (172 mg, 0.696 mmol), 2-bromo-N-(4-fluorophenyl)acetamide (194 mg, 0.835 mmol) and K₂CO₃ (115 mg, 0.835 mmol) in acetonitrile (7 ml) was stirred at room temperature for 1 hour. The solvent was evaporated under reduced pressure and the residue was diluted with ethyl acetate (60 ml), washed with H₂O (10 ml) and brine, dried (MgSO₄) and evaporated under reduced pressure. The residue was purified by column chromatography on silica (ethyl acetate/hexane, 50/50-70/30) to give the title compound (120 mg, 68%).

### Example 168

### 5-((1-(4-Aminophenylsulfonyl)piperidin-4-ylideneaminooxy)methyl)-2-fluorobenzonitrile

was prepared as described in Example 83.

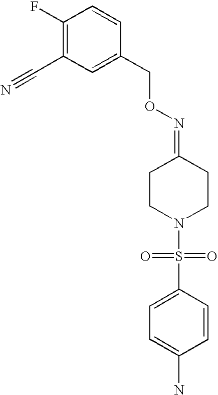

### Example 169

### 2-(Cyclopropylamino)-5-((1-(4-fluorophenylsulfonyl)piperidin-4-ylideneaminooxy)methyl)benzonitrile

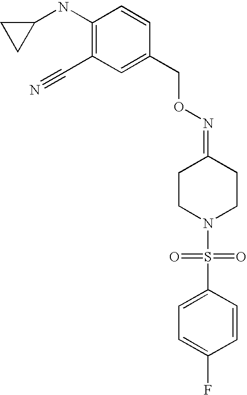

The mixture of 2-fluoro-5-((1-(4-fluorophenylsulfonyl)piperidin-4-ylideneaminooxy)methyl)benzonitrile (200 mg, 0.493 mmol) and cyclopropylamine (2 ml) was refluxed for 4 days. The solvent was evaporated under reduced pressure and the residue was diluted with ethyl acetate (60 ml), washed with H₂O (10 ml) and brine, dried (MgSO₄) and evaporated under reduced pressure. The residue was purified by column chromatography on silica (ethyl acetate/hexane, 15/85-35/65) to give the title compound (65 mg, 30%).

### Example 170

### 2-(Cyclopropylmethylamino)-5-((1-(4-(cyclopropylmethylamino)phenylsulfonyl)piperidin-4-ylideneaminooxy)methyl)benzonitrile

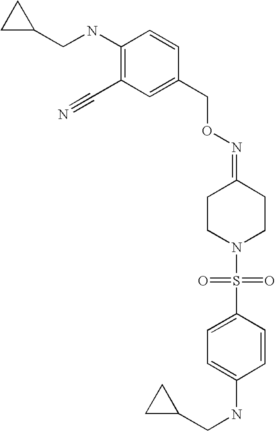

The mixture of 2-fluoro-5-((1-(4-fluorophenylsulfonyl)piperidin-4-ylideneaminooxy)methyl)benzonitrile (200 mg, 0.493 mmol) and aminomethylcyclopropane (0.65 ml) was refluxed for 3 days. The solvent was evaporated under reduced pressure and the residue was diluted with ethyl acetate (60 ml), washed with H₂O (10 ml) and brine, dried (MgSO₄) and evaporated under reduced pressure. The residue was purified by column chromatography on silica (ethyl acetate/hexane, 30/70-60/40) to give the title compound (201 mg, 80%).

### Example 171

### N-(4-(4-(3-Cyano-4-fluorobenzyloxyimino)piperidin-1-ylsulfonyl)phenyl)acetamide

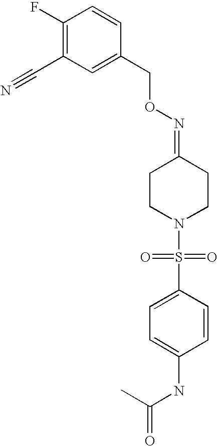

To a solution of 5-((1-(4-Aminophenylsulfonyl)piperidin-4-ylideneaminooxy)methyl)-2-fluorobenzonitrile (100 mg, 0.248 mmol) and triethylamine (0.042 ml, 0.30 mmol) in tetrahydrofuran (3 ml), acetyl chloride (0.088 ml, 1,2 mmol) was added at 0° C. and stirred at room temperature for 2 hours. H₂O (10 ml) was added, extracted with ethyl acetate (30 ml×2), washed with brine, dried (MgSO₄) and evaporated under reduced pressure. The residue was purified by column chromatography on silica (ethyl acetate/hexane, 65/35-100/0) to give the title compound (96 mg, 87%).

### Example 172

### 1-(4-(Trifluoromethoxy)phenylsulfonyl)piperidin-4-one O-(3-methyl-4,5-dihydroisoxazol-5-yl)methyl oxime

was prepared as described in Example 107.

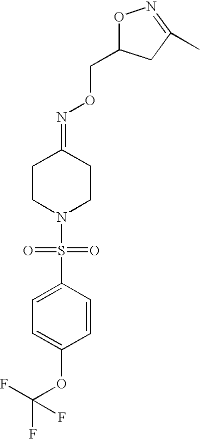

### Example 173

### 6-((1-(4-(Trifluoromethoxy)phenylsulfonyl)piperidin-4-ylideneaminooxy)methyl)picolinonitrile

was prepared as described in Example 107.

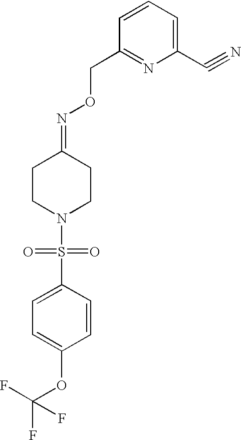

¹H-NMR (DMSO-d₆) δ: 8.02 (1H, t), 7.93 (3H, t), 7.64 (3H, dd), 5.10 (2H, s), 3.14 (4H, t), 2.69 (2H, t), 2.34 (2H, t).

### Example 174

### (R)-1-(4-(trifluoromethoxy)phenylsulfonyl)piperidin-4-one O-morpholin-3-ylmethyl oxime

was prepared as described in Example 107: Starting material was (R)-tert-butyl 3-(aminooxymethyl)morpholine-4-carboxylate (see example A13).

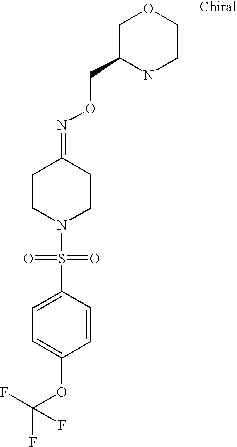

### Example 175

### (S)-1-(4-(trifluoromethoxy)phenylsulfonyl)piperidin-4-one O-morpholin-3-ylmethyl oxime

was prepared as described in Example 107. Starting material was (S)-tert-butyl 3-(aminooxymethyl)morpholine-4-carboxylate (see example A14).

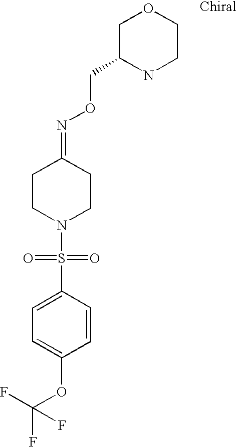

### Example 176

### 2-Fluoro-5-((1-(4-(methylamino)phenylsulfonyl)piperidin-4-ylideneaminooxy)methyl)benzonitrile

was prepared as described in Example 107.

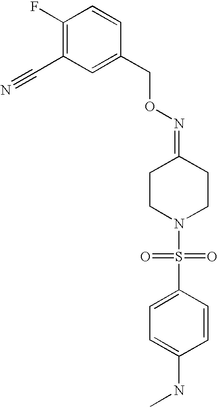

a) To a solution of 5-((1-(4-Aminophenylsulfonyl)piperidin-4-ylideneaminooxy)methyl)-2-fluorobenzonitrile (201 mg, 0.500 mmol) in tetrahydrofuran (6 ml), trifluoroacetic anhydride (0.085 ml, 0.6 mmol) was added at 0° C. and stirred at 0° C. for 30 minutes. The reaction was quenched with saturated aqueous NaHCO₃ solution (10 ml), extracted with diethyl ether (30 ml×2), dried (MgSO4) and evaporated under reduced pressure to give N-(4-(4-(3-cyano-4-fluorobenzyloxyimino)piperidin-1-ylsulfonyl)phenyl)-2,2,2-trifluoroacetamide (248 mg, 100%, colorless solid).

b) To a solution of N-(4-(4-(3-cyano-4-fluorobenzyloxyimino)piperidin-1-ylsulfonyl)phenyl)-2,2,2-trifluoroacetamide (100 mg, 0.200 mmol) and iodomethane (0.050 ml, 0.80 mmol) in acetone (1 ml), potassium hydroxide (45 mg, 0.80 mmol) and H₂O (1 ml) was added at 60° C. and refluxed for 1 hour. The reaction mixture was diluted with 40 ml of ethyl acetate and washed with H₂O (10 ml) and brine. The organic layer was dried over MgSO₄ and concentrated. The residue was purified by column chromatography on silica (ethyl acetate/methanol, 40/60-60/40) to give the title compound (65 mg, 78%).

### Example 177

### 4-((1-(4-(Trifluoromethoxy)phenylsulfonyl)piperidin-4-ylideneaminooxy)methyl)picolinonitrile

was prepared as described in Example 107.

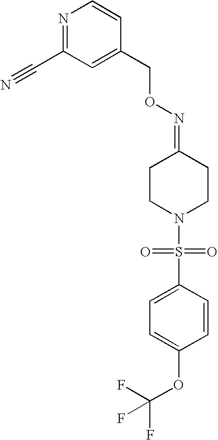

¹H-NMR (DMSO-d₆) δ: 8.69 (1H, d), 7.92-7.91 (3H, m), 7.64-7.62 (3H, m), 5.11 (2H, s), 3.15-3.13 (4H, m), 2.70 (2H, s), 2.34 (2H, s).

### Example 178

### 2-((1-(4-(trifluoromethoxy)phenylsulfonyl)piperidin-4-ylideneaminooxy)methyl)isonicotinonitrile

was prepared as described in Example 107. Starting material was 2-(aminooxymethyl)isonicotinonitrile (see example A15).

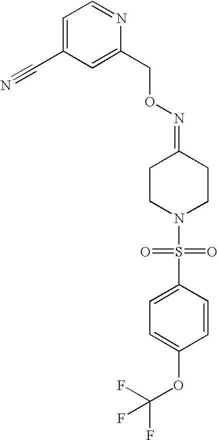

¹H-NMR (DMSO-d₆) δ: 8.77 (1H, d), 7.92 (2H, d), 7.77 (1H, d), 7.72 (1H, s), 7.64 (2H, d), 5.12 (2H, s), 3.15-3.13 (4H, m), 2.71 (2H, t), 2.35 (2H, t).

### Example 179

### 1-(4-(Trifluoromethoxy)phenylsulfonyl)piperidin-4-one O-(4-methoxypyridin-2-yl)methyl oxime

was prepared as described in Example 107. Starting material was O-((4-methoxypyridin-2-yl)methyl)hydroxylamine (see example A16).

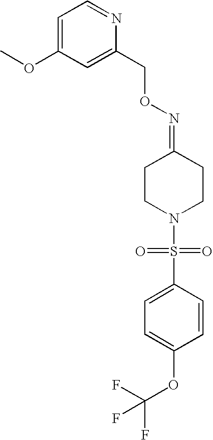

### Example 180

### 5((1-(2-(Bis(4-fluorophenyl)amino)acetyl)piperidin-4-ylideneaminooxy)methyl)-2-fluorobenzonitrile

was prepared as described in Example 153.

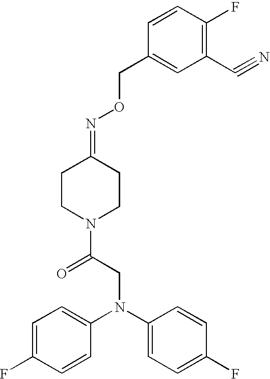

### Example 181

### 5-((1-(4,4-Bis(4-fluorophenyl)butanoyl)piperidin-4-ylideneaminooxy)methyl)-2-fluorobenzonitrile

was prepared as described in Example 153.

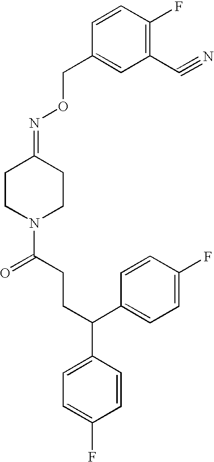

¹H-NMR (DMSO-d₆) δ: 7.88-7.87 (1H, m), 7.75-7.73 (1H, m), 7.52 (1H, t), 7.32 (4H, t), 7.10 (4H, t), 5.04 (2H, s), 4.01 (1H, s), 3.52 (2H, q), 3.37 (2H, d), 2.24 (6H, t).

### Example 182

### 5-((1-(Bis(4-fluorophenyl)methyl)piperidin-4-ylideneaminooxy)methyl)-2-fluorobenzonitrile hydrochloride

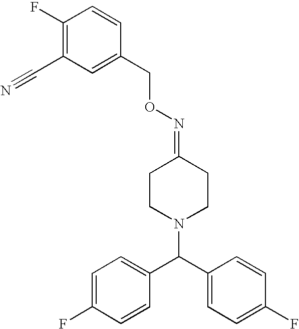

a) The mixture of 2-fluoro-5-((piperidin-4-ylideneaminooxy)methyl)benzonitrile (124 mg, 0.500 mmol), 4,4′-(chloromethylene)bis(fluorobenzene) (122 mg, 0.500 mmol), KI (8 mg, 0.05 mmol) and K₂CO₃ (83 mg, 0.60 mmol) in acetonitrile (5 ml) was refluxed for 14 hours. The reaction was quenched with H₂O, extracted with chloroform (30 ml×2), dried (MgSO₄) and concentrated. The residue was purified by column chromatography on silica (ethyl acetate/methanol, 10/90-35/65) to give 5-((1-(bis(4-fluorophenyl)methyl)piperidin-4-ylideneaminooxy)methyl)-2-fluorobenzonitrile (195 mg, colorless oil). To a solution of this free base in 1,4-dioxane (2 ml), 4N—HCl in 1,4-dioxane (1 ml) was added and stirred at room temperature for 30 minutes. The solvent was removed under reduced pressure to give the title compound (225 mg, 92%).

¹H-NMR (DMSO-d₆) δ: 7.97-7.92 (5H, m), 7.78-7.75 (1H, m), 7.53 (1H, t), 7.33-7.30 (4H, m), 5.76 (1H, d), 5.07 (2H, s), 3.32-2.82 (8H, m).

### Example 183

### N-(Bis-4-fluorophenyl-methyl)-4-(3-cyano-4-fluorobenzyloxyimino)piperidine-1-carboxamide

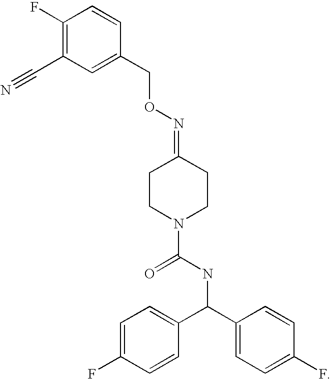

To a solution of bis(4-fluorophenyl)methanamine (110 mg, 0.500 mmol) and pyridine (0.036 ml, 0.500 mmol) in dichloromethane (5 ml), chloroformic acid 4-nitrophenyl ester (101 mg, 0.500 mmol) was added at 0° C. and stirred for 30 minutes. Triethylamine (0.070 ml, 0.50 mmol) and a solution of 2-fluoro-5-((piperidin-4-ylideneaminooxy)methyl)benzonitrile (124 mg, 0.500 mmol) in dichloromethane (3 ml) was added to the reaction mixture and stirred at room temperature for 5 hour. The reaction was quenched with H₂O, extracted with chloroform (30 ml×2), dried (MgSO₄) and concentrated. The residue was purified by column chromatography on silica (ethyl acetate/methanol, 50/50-70/30) to give the title compound (158 mg, 70%).

### Example 184

### 1-(4-(Trifluoromethoxy)phenylsulfonyl)piperidin-4-one O-isoquinolin-1-yl oxime

was prepared as described in Example 107. Starting material was O-(isoquinolin-1-yl)hydroxylamine (see example A17).

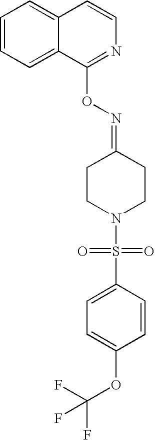

### Example 185

### 1-(Bis(4-fluorophenyl)methyl)piperidin-4-one O-benzhydryl oxime

was prepared as described in Example 183.

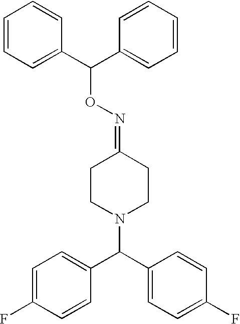

### Example 186

### 3-(Cyclopropyl(1-(4-(trifluoromethoxy)phenylsulfonyl)piperidin-4-ylideneaminooxy)methyl)benzonitrile

was prepared as described in Example 107. Starting material was 3-(aminooxy(cyclopropyl)methyl)benzonitrile (see example A18).

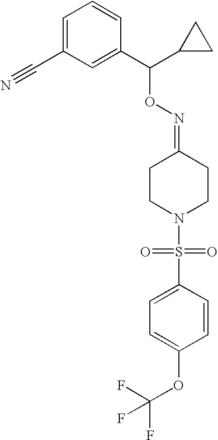

¹H-NMR (DMSO-d₆) δ: 7.90 (2H, d), 7.70 (2H, d), 7.64-7.61 (3H, m), 7.51 (1H, t), 4.42 (1H, d), 3.19-2.98 (4H, m), 2.73-2.66 (2H, m), 2.29-2.23 (2H, m), 1.14-1.10 (1H, m), 0.60-0.37 (4H, m).

### Example 187

### Methyl 6-(4-(3-cyano-4-fluorobenzyloxyimino)piperidin-1-ylsulfonyl)-1H-benzo[d]imidazol-2-ylcarbamate

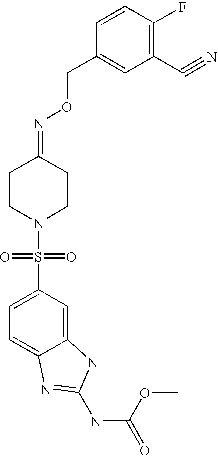

To a solution of 2-fluoro-5-((piperidin-4-ylideneaminooxy)methyl)benzonitrile (247 mg, 1.00 mmol) and triethylamine (0.35 ml, 2.5 mmol) in dichloromethane (5 ml), methyl 6-(chlorosulfonyl)-1H-benzo[d]imidazol-2-ylcarbamate (326 mg, 1.00 mmol) was added at 0° C. and stirred at room temperature for 3 hours. The reaction was quenched with H₂O, extracted with dichloromethane, dried (MgSO4) and evaporated under reduced pressure. The residual solid was triturated with diethyl ether/hexane (4/1) to give the title compound (466 mg, 93%).

### Example 188

### 5-((1-(2-Amino-1H-benzo[d]imidazol-6-ylsulfonyl)piperidin-4-ylideneaminooxy)methyl)-2-fluorobenzonitrile

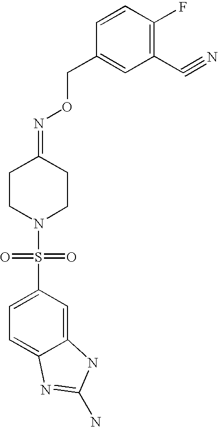

To a solution of methyl 6-(4-(3-cyano-4-fluorobenzyloxyimino)piperidin-1-ylsulfonyl)-1H-benzo[d]imidazol-2-ylcarbamate (150 mg, 0.300 mmol) in ethylene glycol (1.5 ml) and H₂O (0.4 ml), potassium hydroxide (50 mg, 0.90 mmol) was added and stirred at 95° C. for 90 minutes. The reaction was quenched with H₂O and the precipitated material was collected, then purified by column chromatography on silica (ethyl acetate/methanol, 90/10-70/30) to give the title compound (50 mg, 40%).

### Example 189

### 1-(4-(Trifluoromethoxy)phenylsulfonyl)piperidin-4-one O-(4-methoxyquinolin-2-yl)methyl oxime

was prepared as described in Example 107. Starting material was O-((4-methoxyquinolin-2-yl)methyl)hydroxylamine (see example A19).

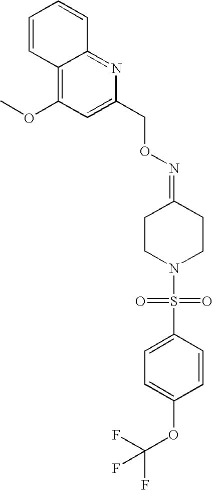

¹H-NMR (DMSO-d₆) δ: 8.09 (1H, d), 7.91-7.87 (3H, m), 7.72 (1H, dd), 7.64 (2H, d), 7.53 (1H, dd), 6.88 (1H, s), 5.17 (2H, s), 3.95 (3H, s), 3.14-3.13 (4H, m), 2.76 (2H, t), 2.36 (2H, t).

### Example 190

### 1-(4-(Trifluoromethoxy)phenylsulfonyl)piperidin-4-one O-(4-fluorophenyl)(6-methoxypyridin-3-yl)methyl oxime

was prepared as described in Example 107. Starting material was O-((4-fluorophenyl)(6-methoxypyridin-3-yl)methyl)hydroxylamine (see example A20).

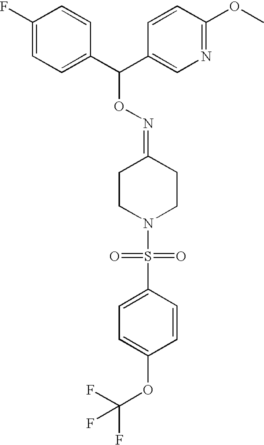

### Example 191

### 3-(2-Hydroxy-1-(1-(4-(trifluoromethoxy)phenylsulfonyl)piperidin-4-ylideneaminooxy)ethyl)benzonitrile

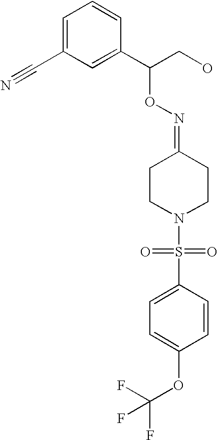

To a solution of 3-(2-(tert-butyldimethylsilyloxy)-1-(1-(4-(trifluoromethoxy)phenylsulfonyl)piperidin-4-ylideneaminooxy)ethyl)benzonitrile (prepared as described in Example 107 starting from 3-(1-(aminooxy)-2-(tert-butyldimethylsilyloxy)ethyl)benzonitrile (see example A21), 154 mg, 0.250 mmol) in tetrahydrofuran (5 ml), tetra-n-butylammonium fluoride hydrate (131 mg, 0.500 mmol) was added and stirred at room temperature for 30 minutes. The solvent was evaporated under reduced pressure and the residue was diluted with ethyl acetate (60 ml), washed with saturated aqueous NaHCO₃ solution (10 ml) and brine, dried (MgSO₄) and evaporated under reduced pressure. The residue was purified by column chromatography on silica (ethyl acetate/hexane, 55/45-75/25) to give the title compound (104 mg, 86%).

¹H-NMR (DMSO-d₆) δ: 7.91 (2H, d), 7.69-7.61 (5H, m), 7.51 (1H, t), 5.06-5.03 (1H, m), 4.92-4.90 (1H, m), 3.64-3.60 (2H, m), 3.11-3.10 (4H, m), 2.76-2.70 (2H, m), 2.31-2.29 (2H, m).

### Example 192

### 1-(4-(Trifluoromethoxy)phenylsulfonyl)piperidin-4-one O-(5-chlorobenzo[d]oxazol-2-yl)methyl oxime

was prepared as described in Example 107. Starting material was O-((5-chlorobenzo[d]oxazol-2-yl)methyl)hydroxylamine (see example A22).

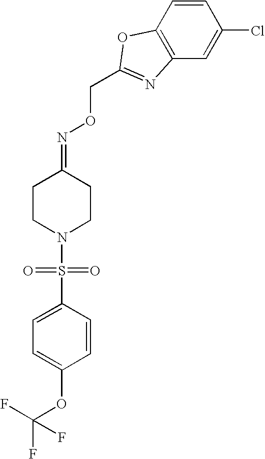

### Example 193

### 2-((1-(4-(Trifluoromethoxy)phenylsulfonyl)piperidin-4-ylideneaminooxy)methyl)quinolin-4(1H)-one

was prepared as described in Example 145.

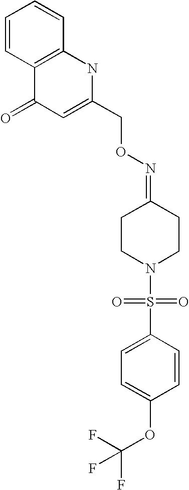

### Example 194

### 2-((1-(4-(Trifluoromethoxy)phenylsulfonyl)piperidin-4-ylideneaminooxy)methyl)pyridin-4(1H)-one

was prepared as described in Example 145.

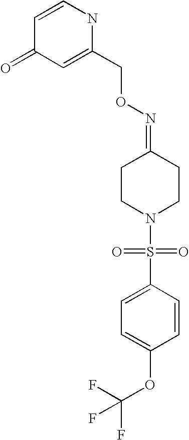

### Example 195

### 1-(4-(Trifluoromethoxy)phenylsulfonyl)piperidin-4-one O-benzhydryl oxime

was prepared as described in Example 107.

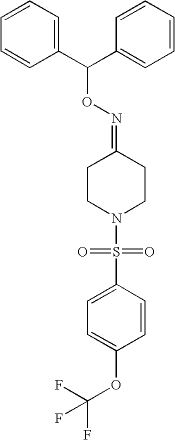

### Example 196

### 3-((1-(4-Fluorophenylsulfonyl)piperidin-4-ylideneaminooxy)methylbenzonitrile

was prepared as described in EXAMPLE 50

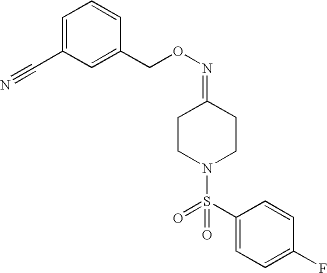

### Example 197

### 3-((1-(4-(Difluoromethoxy)phenylsulfonyl)piperidin-4-ylideneaminooxy)methyl)benzonitrile

was prepared as described in EXAMPLE 50

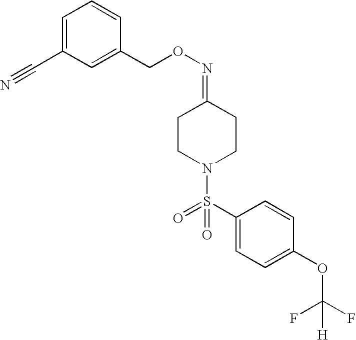

### Example 198

### 3-((1-(4-Methoxyphenylsulfonyl)piperidin-4-ylideneaminooxy)methyl)benzonitrile

was prepared as described in EXAMPLE 50

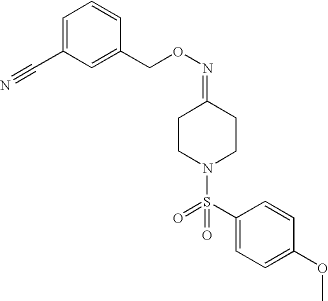

### Example 199

### 3-((1-(4-Cyanophenylsulfonyl)piperidin-4-ylideneaminooxymethyl)benzonitrile

was prepared as described in EXAMPLE 50

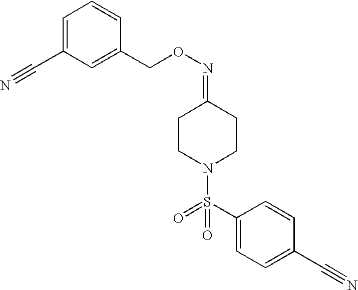

### Example 200

### N-Benzhydryl-4-(benzhydryloxyimino)piperidine-1-carboxamide

was prepared as described in EXAMPLE 183

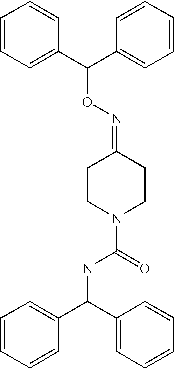

### Example 201

### N-Benzhydryl-4-(3-cyanobenzyloxyimino)piperidine-1-carboxamide

was prepared as described in EXAMPLE 183

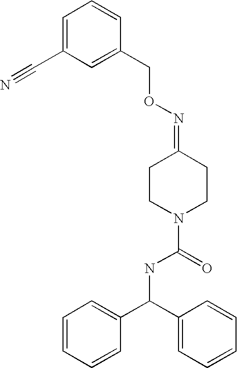

### Example 202

### 4-(3-Cyanobenzyloxyimino)-N-(9H-fluoren-9-yl)piperidine-1-carboxamide

was prepared as described in EXAMPLE 183

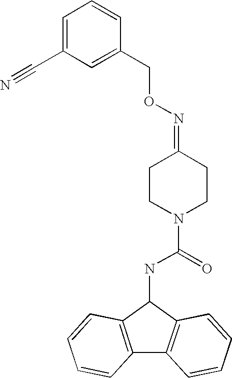

### Example 203

### 1-(4-(Trifluoromethoxy)phenylsulfonyl)piperidin-4-one O-(1-(2-hydroxyethyl)-1H-benzo[d]imidazol-2-yl)methyl oxime

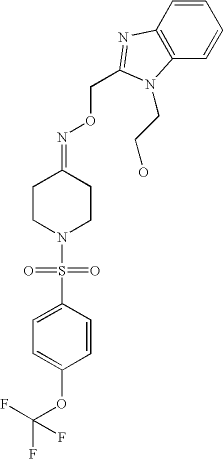

a) To a solution of 1-(4-(trifluoromethoxy)phenylsulfonyl)piperidin-4-one O-(1H-benzo[d]imidazol-2-yl)methyl oxime (134 mg, 0.290 mmol) in N,N-dimethylformamide (2 ml), NaH (60%, 13 mg, 0.31 mmol) was added at 0° C. and stirred at room temperature for 10 minutes. tert-Butyl-(2-bromoethoxy)dimethylsilane (0.067 ml, 0.31 mmol) was added to the reaction mixture and stirred at room temperature for 3 hours. The reaction was quenched with H₂O and extracted with ethyl acetate, dried (MgSO₄) and evaporated under reduced pressure. The residue was purified by column chromatography on silica (ethyl acetate/hexane, 30/70-50/50) to give 1-(4-(trifluoromethoxy)phenylsulfonyl)piperidin-4-one O-(1-(2-(tert-butyldimethylsilyloxy)ethyl)-1H-benzo[d]imidazol-2-yl)methyl oxime (137 mg, 75%, pale yellow oil).

b) To a solution of 1-(4-(trifluoromethoxy)phenylsulfonyl)piperidin-4-one O-(1-(2-(tert-butyldimethylsilyloxy)ethyl)-1H-benzo[d]imidazol-2-yl)methyl oxime (137 mg, 0.220 mmol) in tetrahydrofuran (3 ml), tetra-n-butylammonium fluoride hydrate (1 M solution in tetrahydrofuran, 0.26 ml, 0.26 mmol) was added at 0° C. and stirred at room temperature for 15 minutes. The reaction was quenched with H₂O and extracted with ethyl acetate, dried (MgSO₄) and evaporated under reduced pressure. The residue was purified by column chromatography on silica (chloroform/methanol, 95/5-90/10) to give the title compound (94 mg, 80%).

### Example 204

### 1-(4-(Trifluoromethoxy)phenylsulfonyl)piperidin-4-one O-(1H-indol-2-yl)methyl oxime

was prepared as described in Example 107. Starting material was O-((1H-indol-2-yl)methyl)hydroxylamine (see example A23).

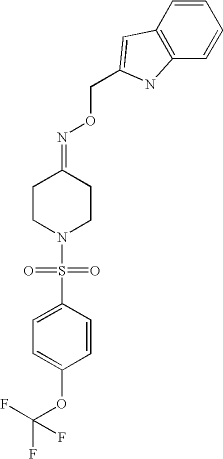

### Example 205

### 3-((1-(3,4-Dimethoxyphenylsulfonyl)piperidin-4-ylideneaminooxy)methyl)benzonitrile

was prepared as described in EXAMPLE 50

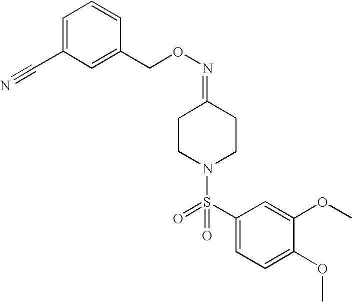

### Example 206

### 3-((1-(2,5-Dimethoxyphenylsulfonyl)piperidin-4-ylideneaminooxy)methyl)benzonitrile

was prepared as described in EXAMPLE 50

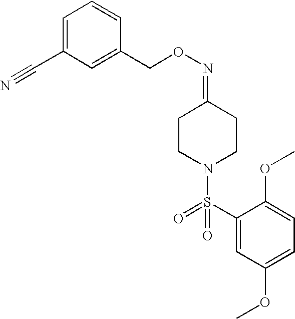

### Example 207

### 5-(4-(3-Cyanobenzyloxyimino)piperidin-1-ylsulfonyl)-2-fluorobenzonitrile

was prepared as described in EXAMPLE 50

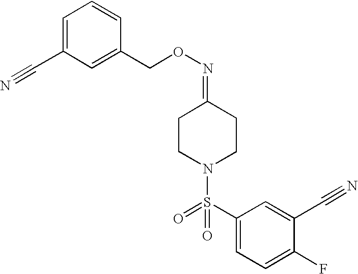

### Example 208

### 3-((1-(3,5-Difluorophenylsulfonyl)piperidin-4-ylideneaminooxy)methyl)benzonitrile

was prepared as described in EXAMPLE 50

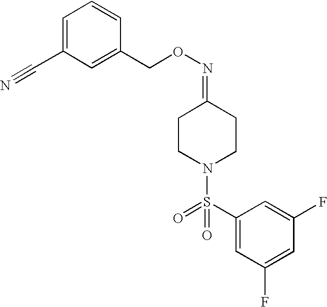

### Example 209

### 3-((1-(2,4-Difluorophenylsulfonyl)piperidin-4-ylideneaminooxy)methyl)benzonitrile

was prepared as described in EXAMPLE 50

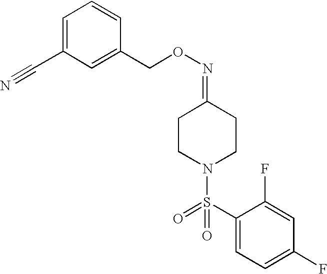

### Example 210

### 3-((1-(3,4-Difluorophenylsulfonyl)piperidin-4-ylideneaminooxy)methyl)benzonitrile

was prepared as described in EXAMPLE 50

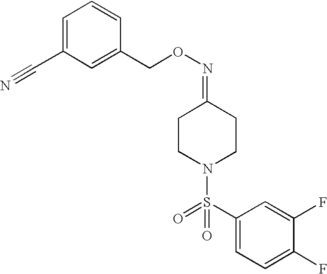

### Example 211

### 3-((1-(2,5-Difluorophenylsulfonyl)piperidin-4-ylideneaminooxy)methyl)benzonitrile

was prepared as described in EXAMPLE 50

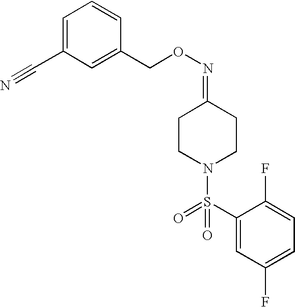

### Example 212

### Methyl 3-((1-(4-(trifluoromethoxy)phenylsulfonyl)piperidin-4-ylideneaminooxy)methyl)benzoate

was prepared as described in Example 107.

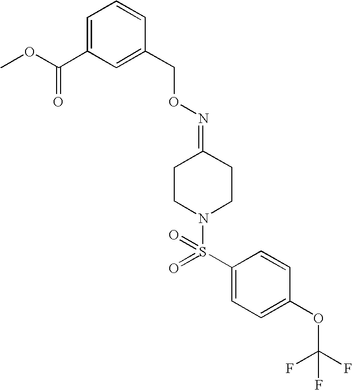

### Example 213

### 3-((1-(4-(Trifluoromethoxy)phenylsulfonyl)piperidin-4-ylideneaminooxy)methyl)benzoic acid

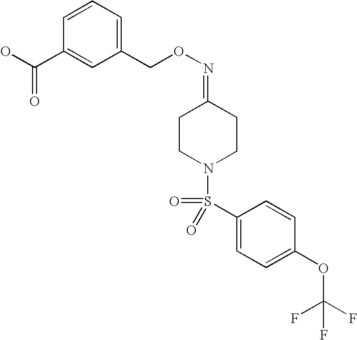

To a solution of methyl 3-((1-(4-(trifluoromethoxy)phenylsulfonyl)piperidin-4-ylideneaminooxy)methyl)benzoate (1.08 g, 2.22 mmol) in methanol (15 ml), aqueous sodium hydroxide solution (4 M, 1.10 ml, 4.40 mmol) was added and refluxed for 1 hour. HCl (1 M solution, 5 ml) and H₂O (10 ml) was added dropwise to the reaction mixture, and the resulting precipitate was collected, washed with H₂O (10 ml×3) and diethyl ether (5 ml×3) to give the title compound (875 mg, 83%)

### Example 214

### 5-((4-Fluorophenyl)(1-(4-(trifluoromethoxy)phenylsulfonyl)piperidin-4-ylideneaminooxy)methyl)pyridin-2(1H)-one

was prepared as described in Example 145.

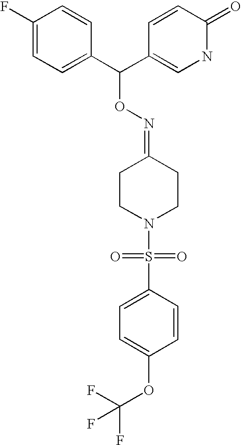

¹H-NMR (DMSO-d₆) δ: 11.50 (1H, br s), 7.90 (2H, d), 7.63 (2H, d), 7.32-7.29 (3H, m), 7.23 (1H, s), 7.14 (2H, t), 6.26 (1H, d), 5.93 (1H, s), 3.12-3.07 (4H, m), 2.72-2.67 (2H, m), 2.31 (2H, t).

### Example 215

### 1-(4-(Trifluoromethoxy)phenylsulfonyl)piperidin-4-one O-(4-fluorophenyl)(1-methyl-1H-imidazol-5-yl)methyl oxime

was prepared as described in Example 107. Starting material was O-((4-Fluorophenyl)(1-methyl-1H-imidazol-5-yl)methyl)hydroxylamine (see example A24).

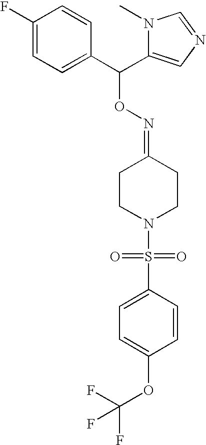

### Example 216

### 1-(4-(Trifluoromethoxy)phenylsulfonyl)piperidin-4-one O-(4-fluorophenyl)(piperidin-4-yl)methyl oxime

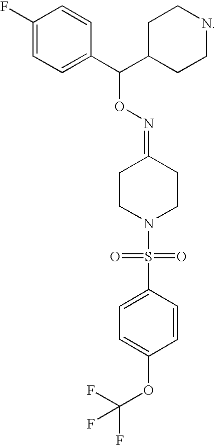

To a solution of tert-Butyl 4-((4-fluorophenyl)(1-(4-(trifluoromethoxy)phenylsulfonyl)piperidin-4-ylideneaminooxy)methyl)piperidine-1-carboxylate (prepared as described in Example 107 starting from tert-butyl 4-(aminooxy(4-fluorophenyl)methyl)piperidine-1-carboxylate (see example A25), 200 mg, 0.317 mmol) in dichloroethane (2 ml), trifluoroacetic acid (1 ml) was added at 0° C. and stirred at 0° C. for 2 hours. The reaction mixture was concentrated under reduced pressure and the residue was diluted with ethyl acetate, washed with saturated aqueous NaHCO₃ solution and brine, dried (MgSO₄) and evaporated under reduced pressure to give the title compound (172 mg, 100%).

¹H-NMR (DMSO-d₆) δ: 7.90 (2H, d), 7.63 (2H, d), 7.18-7.07 (4H, m), 4.72 (1H, d), 3.06-2.90 (6H, m), 2.67 (2H, t), 2.39-2.19 (4H, m), 1.74-1.68 (2H, m), 1.12-1.04 (3H, m).

### Example 217

### 1-(4-(Trifluoromethoxy)phenylsulfonyl)piperidin-4-one O-(1H-indol-3-yl)methyl oxime

was prepared as described in Example 107. Starting material was O-((1H-indol-3-yl)methyl)hydroxylamine (see example A26).

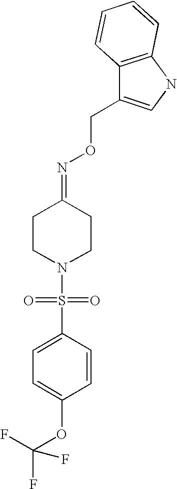

### Example 218

### 3-((1-(4-(2-Methoxyethylamino)phenylsulfonyl)piperidin-4-ylideneaminooxy)methyl)benzonitrile

was prepared as described in EXAMPLE 86

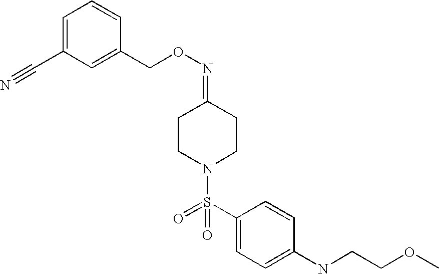

### Example 219

### 3-((1-(4-(2-Methoxyethoxy)phenylsulfonyl)piperidin-4-ylideneaminooxy)methyl)benzonitrile

was prepared as described in EXAMPLE 91

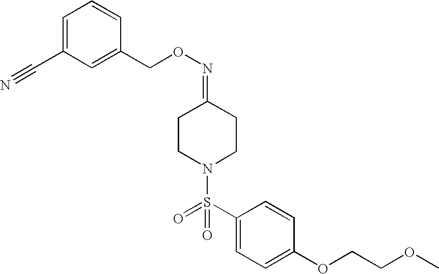

### Example 220

### 3-((1-(4-(2-Hydroxyethylamino)phenylsulfonyl)piperidin-4-ylideneaminooxy)methyl)benzonitrile

was prepared as described in EXAMPLE 86

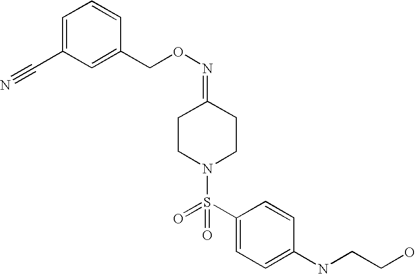

### Example 221

### 3-((1-(4-(2-Hydroxyethoxy)phenylsulfonyl)piperidin-4-ylideneaminooxy)methyl)benzonitrile

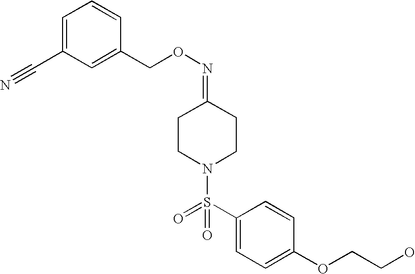

To a solution of 3-((1-(4-(2-(tetrahydro-2H-pyran-2-yloxy)ethoxy)phenylsulfonyl)piperidin-4-ylideneaminooxy)methyl)benzonitrile (prepared as described in EXAMPLE 91, 466 mg, 0.516 mmol) in tetrahydrofuran (1.5 ml) and methanol (5 mL) was added a catalytic amount of camphorsulfonic acid (50 mg) at 0° C. After being stirred for 2 hours at room temperature, the reaction mixture was diluted with ethyl acetate and poured into saturated aqueous NaHCO₃ at 0° C. The aqueous layer was extracted with ethyl acetate and the combined extracts were washed with H₂O and brine, dried over Na₂SO4, filtered and evaporated in vacuo. The residue was purified by column chromatography on silica (ethyl acetate/hexane, 50/50-80/20) and recrystallized from isopropylether/chloroform to give the title compound (184 mg, 83%).

### Example 222

### 3-((1-(4-(Cyclopropylmethylamino)phenylsulfonyl)piperidin-4-ylideneaminooxy)methyl)benzonitrile

was prepared as described in EXAMPLE 86

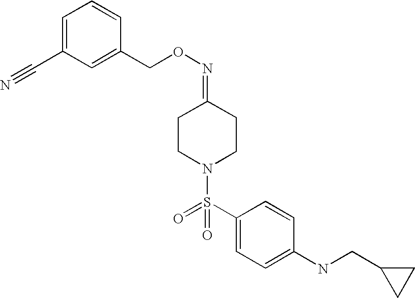

### Example 223

### 3-((1-(4-(2-Hydroxy-2-methylpropylamino)phenylsulfonyl)piperidin-4-ylideneaminooxy)methyl)benzonitrile

was prepared as described in EXAMPLE 86

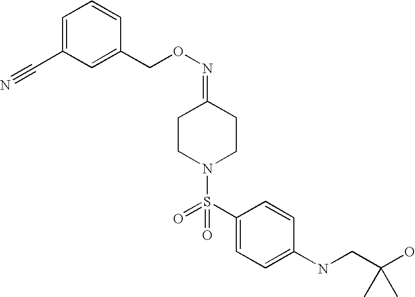

### Example 224

### N-(3,5-Bis(trifluoromethylphenyl)-4-(3-(trifluoromethyl)benzyloxyimino)piperidine-1-carboxamide

was prepared as described in EXAMPLE 183

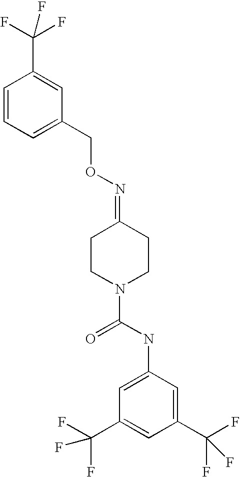

### Example 225

### 4-(Bis(4-fluorophenyl)methoxyimino)-N-(3,5-bis(trifluoromethyl)phenyl)piperidine-1-carboxamide

was prepared as described in EXAMPLE 183

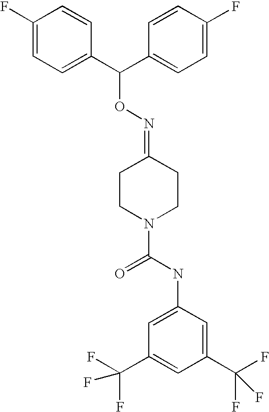

### Example 226

### N-(2,4-Difluorophenyl)-3-oxo-3-(4-(3-(trifluoromethyl)benzyloxyimino)piperidin-1-yl)propanamide

was prepared as described in Example 153.

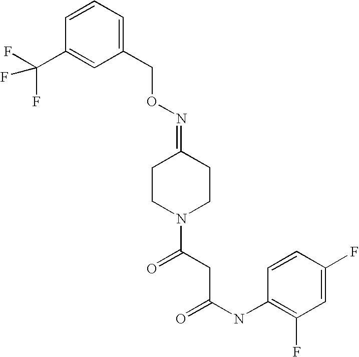

### Example 227

### 3-(4-(Bis(4-fluorophenyl)methoxyimino)piperidin-1-yl)-N-(2,4-difluorophenyl)-3-oxopropanamide

was prepared as described in Example 153.

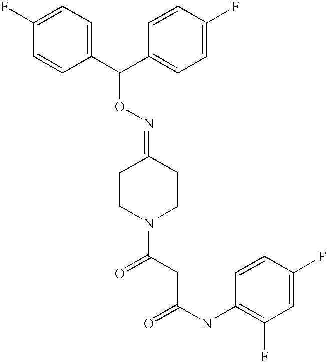

### Example 228

### 1-(2,4-Dichlorophenylsulfonyl)piperidin-4-one O-bis(4-fluorophenyl)methyl oxime

was prepared as described in EXAMPLE 50

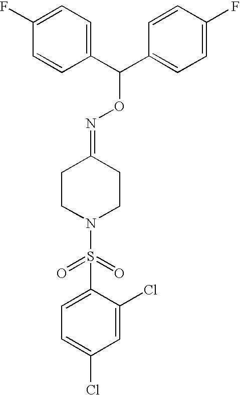

### Example 229

### 2-(4-(Bis(4-fluorophenyl)methoxyimino)piperidin-1-ylsulfonyl)benzonitrile

was prepared as described in EXAMPLE 50

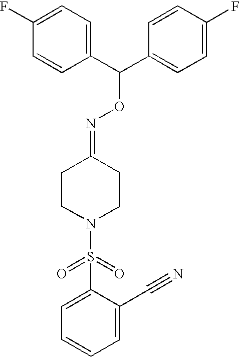

### Example 230

### 1-(o-Tolylsulfonyl)piperidin-4-one O-bis(4-fluorophenyl)methyl oxime

was prepared as described in EXAMPLE 50

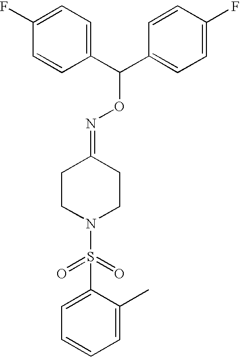

### Example 231

### 1-(4-Butylphenylsulfonyl)piperidin-4-one O-bis(4-fluorophenyl)methyl oxime

was prepared as described in EXAMPLE 50

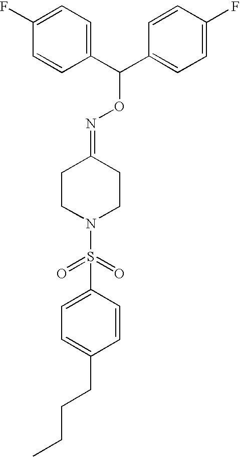

### Example 232

### 1-(4-(Difluoromethoxy)phenylsulfonyl)piperidin-4-one O-bis(4-fluorophenyl)methyl oxime

was prepared as described in EXAMPLE 50

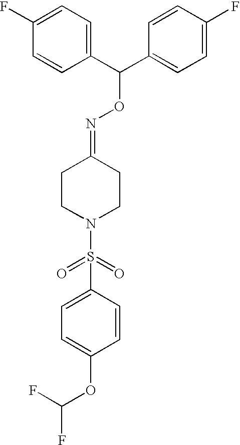

### Example 233

### 1-(3-(Difluoromethoxy)phenylsulfonyl)piperidin-4-one O-bis(4-fluorophenyl)methyl oxime

was prepared as described in EXAMPLE 50

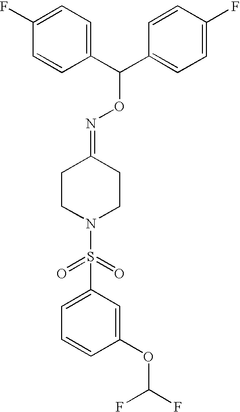

### Example 234

### 1-(1-Methyl-1H-indol-4-ylsulfonyl)piperidin-4-one O-bis(4-fluorophenyl)methyl oxime

was prepared as described in EXAMPLE 50

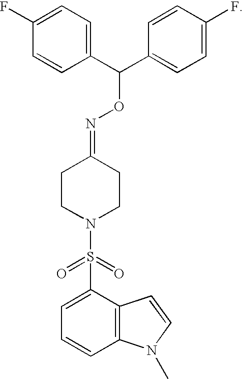

### Example 235

### 1-(4-(1H-Pyrazol-1-yl)phenylsulfonyl)piperidin-4-one O-bis(4-fluorophenyl)methyl oxime

was prepared as described in EXAMPLE 50

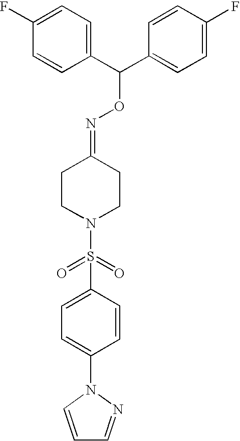

### Example 236

### 1-(Benzo[d][1,3]dioxol-5-ylsulfonyl)piperidin-4-one O-bis(4-fluorophenyl)methyl oxime

was prepared as described in EXAMPLE 50

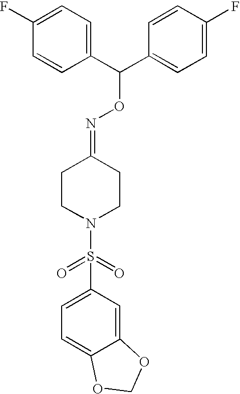

### Example 237

### 1-(4-Methyl-3,4-dihydro-2H-benzo[b][1,4]oxazin-7-ylsulfonyl)piperidin-4-one O-bis(4-fluorophenyl)methyl oxime

was prepared as described in EXAMPLE 50

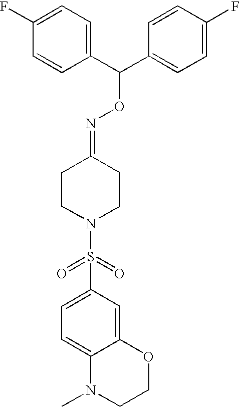

### Example 238

### 1-(2-Methoxyphenylsulfonyl)piperidin-4-one O-bis(4-fluorophenyl)methyl oxime

was prepared as described in EXAMPLE 50

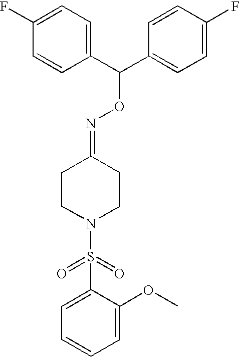

### Example 239

### 3-((1-(4-(Trifluoromethoxy)phenylsulfonyl)piperidin-4-ylideneaminooxy)methyl)benzamide

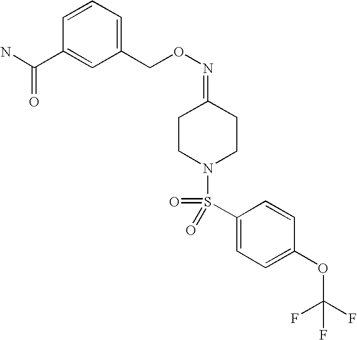

To a solution of 3-((1-(4-(Trifluoromethoxy)phenylsulfonyl)piperidin-4-ylideneaminooxy)methyl)benzoic acid (150 mg, 0.318 mmol), 1-hydroxybenzotriazole (46.4 mg, 0.343 mmol), NH4Cl (34.9 mg, 0.652 mmol) and N,N-diisopropylethylamine (0.220 ml, 1.26 mmol) in N,N-dimethylformamide, N-ethyldimethylaminopropylcarbodiimide hydrochloride (74.5 mg, 0.389 mmol) was added and stirred at room temperature for 88 hours. H₂O (3 ml) was added dropwise to the reaction mixture at room temperature and stirred at 0° C. for 30 minutes. The resulting precipitate was collected, washed with H₂O (5 ml×3) and dried under reduced pressure at 50° C. to give the title compound (135 mg, 90%).

### Example 240

### N-Methyl-3-((1-(4-(trifluoromethoxy)phenylsulfonyl)piperidin-4-ylideneaminooxy)methyl)benzamide

was prepared as described in EXAMPLE 239

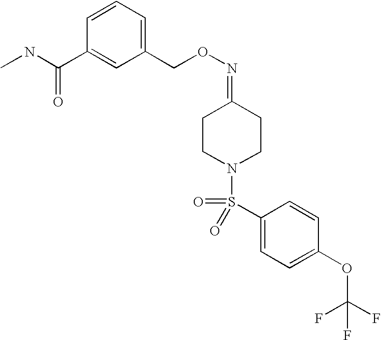

### Example 241

### N,N-Dimethyl-3-((1-(4-(trifluoromethoxy)phenylsulfonyl)piperidin-4-ylideneaminooxy)methyl)benzamide

was prepared as described in EXAMPLE 239

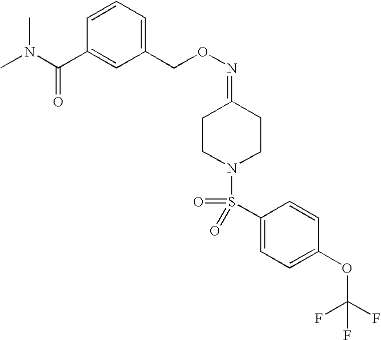

### Example 242

### 1-(3,5-Dimethylisoxazol-4-ylsulfonyl)piperidin-4-one O-bis(4-fluorophenyl)methyl oxime

was prepared as described in EXAMPLE 50

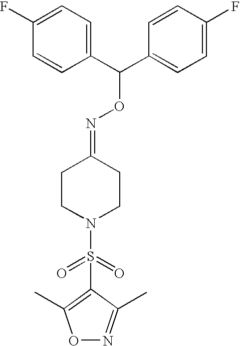

### Example 243

### 1-(3,4-Difluorophenylsulfonyl)piperidin-4-one O-bis(4-fluorophenyl)methyl oxime

was prepared as described in EXAMPLE 50

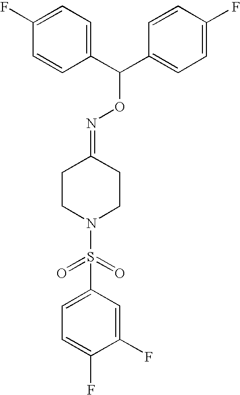

### Example 244

### 1-(3,5-Difluorophenylsulfonyl)piperidin-4-one O-bis(4-fluorophenyl)methyl oxime

was prepared as described in EXAMPLE 50

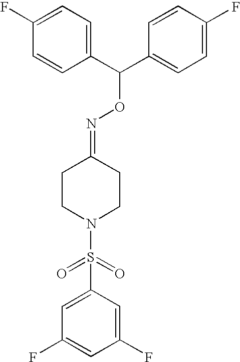

### Example 245

### 1-(2,4-Difluorophenylsulfonyl)piperidin-4-one O-bis(4-fluorophenyl)methyl oxime

was prepared as described in EXAMPLE 50

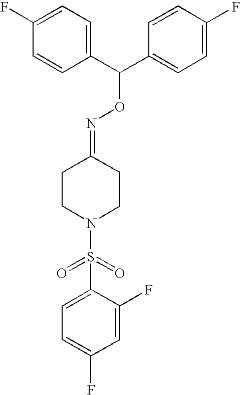

### Example 246

### 1-(4-Fluorophenylsulfonyl)piperidin-4-one O-bis(4-fluorophenyl)methyl oxime

was prepared as described in EXAMPLE 50

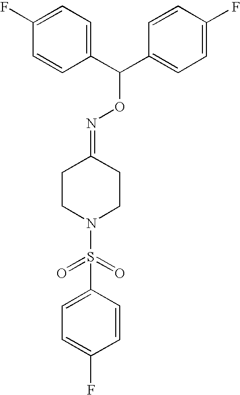

### Example 247

### 1-(4-Butylphenylsulfonyl)piperidin-4-one O-3-chloro-5-fluorobenzyl oxime

was prepared as described in EXAMPLE 65

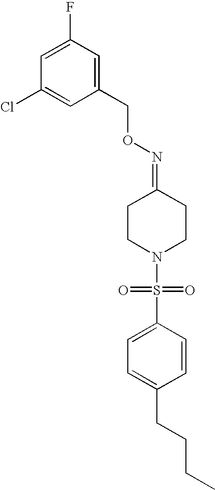

### Example 248

### 1-(4-Butylphenylsulfonyl)piperidin-4-one O-cyclopropylmethyl oxime

was prepared as described in EXAMPLE 65

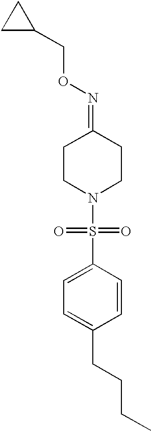

### Example 249

### 1-(4-(Trifluoromethoxy)phenylsulfonyl)piperidin-4-one O-cyclopropylmethyl oxime

was prepared as described in EXAMPLE 65

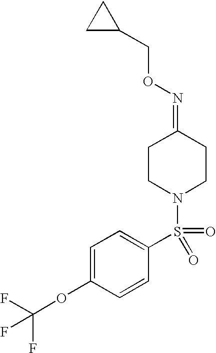

### Example 250

### 1-(3-(Trifluoromethyl)-phenylsulfonyl)piperidin-4-one O-cyclopropylmethyl oxime

was prepared as described in EXAMPLE 65

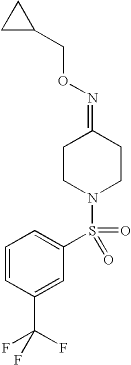

### Example 251

### 1-(Quinolin-8-ylsulfonyl)piperidin-4-one O-bis(4-fluorophenyl)methyl oxime

was prepared as described in EXAMPLE 50

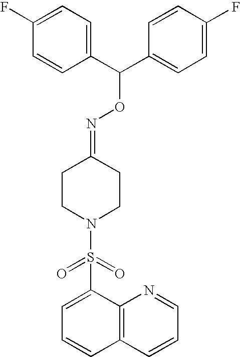

### Example 252

### 1-(Thiophen-2-ylsulfonyl)piperidin-4-one O-bis(4-fluorophenyl)methyl oxime

was prepared as described in EXAMPLE 50

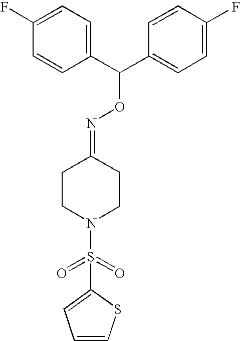

### Example 253

### 4-(Bis(4-fluorophenyl)methoxyimino)-N,N-dimethylpiperidine-1-sulfonamide

was prepared as described in EXAMPLE 50

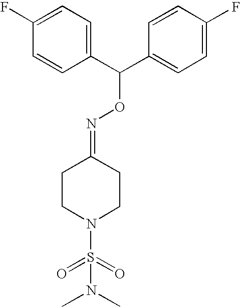

### Example 254

### 1-(1-Methyl-1H-imidazol-4-ylsulfonyl)piperidin-4-one O-bis(4-fluorophenyl)methyl oxime

was prepared as described in EXAMPLE 50

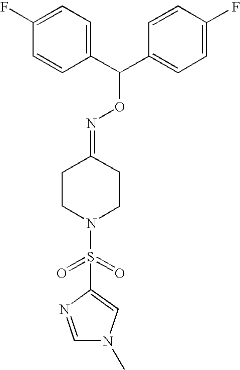

### Example 255

### 1-(Cyclopropylsulfonyl)-piperidin-4-one O-bis(4-fluorophenyl)methyl oxime

was prepared as described in EXAMPLE 50

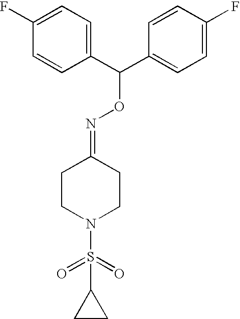

### Example 256

### 4-(4-(Bis(4-fluorophenyl)methoxyimino)piperidin-1-ylsulfonyl)benzonitrile

was prepared as described in EXAMPLE 50

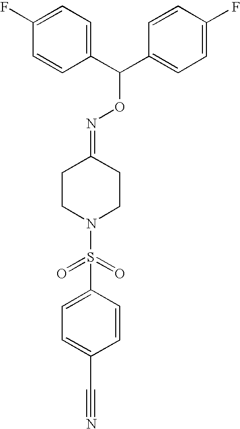

### Example 257

### 5-(4-(Bis(4-fluorophenyl)methoxyimino)piperidin-1-ylsulfonyl)-2-fluorobenzonitrile

was prepared as described in EXAMPLE 50

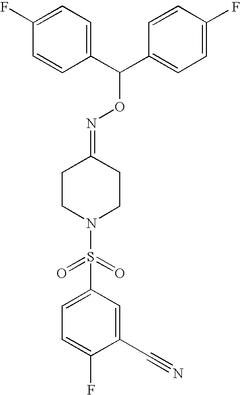

### Example 258

### 1-(2,5-Dimethoxyphenylsulfonyl)piperidin-4-one O-bis(4-fluorophenyl)methyl oxime

was prepared as described in EXAMPLE 50

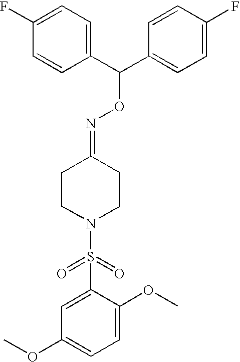

### Example 259

### 1-(2,5-Difluorophenylsulfonyl)piperidin-4-one O-bis(4-fluorophenyl)methyl oxime

was prepared as described in EXAMPLE 50

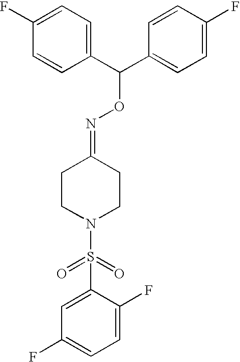

### Example 260

### 1-(4-Methoxyphenylsulfonyl)piperidin-4-one O-bis(4-fluorophenyl)methyl oxime

was prepared as described in EXAMPLE 50

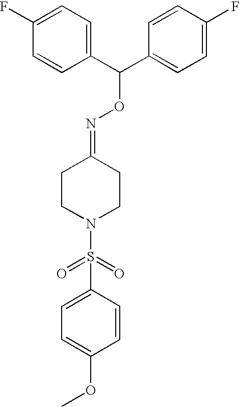

### Example 261

### 1-(2,3-Dihydrobenzo[b][1,4]dioxin-5-ylsulfonyl)piperidin-4-one O-bis(4-fluorophenyl)methyl oxime

was prepared as described in EXAMPLE 50

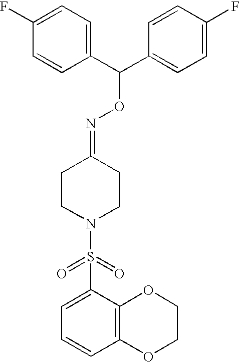

### Example 262

### 1-(1,2-Dimethyl-1H-imidazol-4-ylsulfonyl)-piperidin-4-one O-bis(4-fluorophenyl)methyl oxime

was prepared as described in EXAMPLE 50

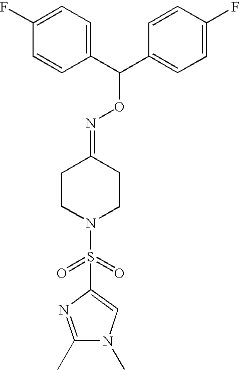

### Example 263

### (S)-1-(4-(trifluoromethoxy)phenylsulfonyl)piperidin-4-one O-indolin-2-ylmethyl oxime

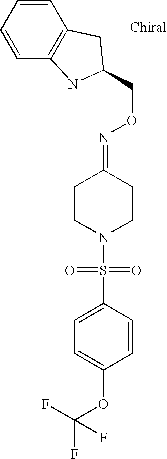

To a solution of (S)-tert-butyl 2-((1-(4-(trifluoromethoxy)phenylsulfonyl)piperidin-4-ylideneaminooxy)methyl)indoline-1-carboxylate (prepared as described in Example 107 starting from (S)-tert-butyl 2-(aminooxymethyl)indoline-1-carboxylate (see example A27), 160 mg, 0.280 mmol) in dichloroethane (2 ml), trifluoroacetic acid (0.42 ml, 5.62 mmol) was added and stirred at room temperature for 45 minutes. The reaction mixture was quenched with ammonia solution at 0° C., extracted with chloroform, dried (MgSO₄) and evaporated under reduced pressure. The residue was purified by column chromatography on silica (ethyl acetate/hexane, 20/80-60/40) to give the title compound (125 mg, 95%).

¹H-NMR (DMSO-d₆) δ: 7.91 (2H, d), 7.65 (2H, d), 6.92 (1H, d), 6.77 (1H, d), 6.40 (2H, dd), 5.55 (1H, s), 3.92 (3H, s), 3.09-3.00 (5H, m), 2.55 (3H, t), 2.32 (2H, t).

### Example 264

### 1-(4-(Trifluoromethoxy)phenylsulfonyl)piperidin-4-one O-(1H-benzo[d]imidazol-5-yl)methyl oxime

was prepared as described in Example 107. Starting material was O-((1H-benzo[d]imidazol-6-yl)methyl)hydroxylamine (see example A28).

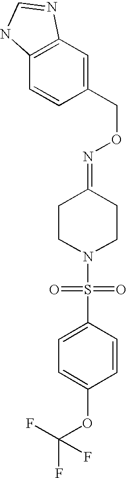

### Example 265

### 1-(4-(Trifluoromethoxy)phenylsulfonyl)piperidin-4-one O-benzo[d][1,3]dioxol-2-ylmethyl oxime

was prepared as described in Example 107. Starting material was O-(benzo[d][1,3]dioxol-2-ylmethyl)hydroxylamine (see example A29).

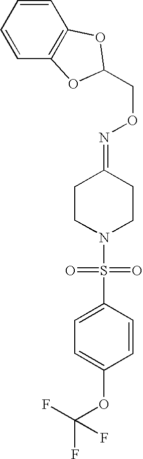

¹H-NMR (DMSO-d₆) δ: 7.88 (2H, d), 7.69 (2H, d), 6.74 (2H, dd), 6.49 (2H, dd), 6.40 (1H, t), 4.24 (2H, d), 3.01 (2H, t), 2.71 (2H, t), 2.30 (4H, td).

### Example 266

### 1-(4-(Trifluoromethoxy)phenylsulfonyl)piperidin-4-one O-(6-fluoro-1H-benzo[d]imidazol-2-yl)methyl oxime

was prepared as described in Example 162. Starting material was tert-Butyl 2-(aminooxymethyl)-5-fluoro-1H-benzo[d]imidazole-1-carboxylate (see example A30).

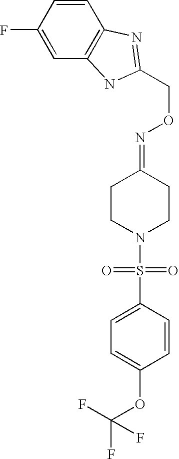

### Example 267

### 3-(4-(Bis(4-fluorophenyl)methoxyimino)piperidin-1-ylsulfonyl)benzonitrile

was prepared as described in EXAMPLE 50

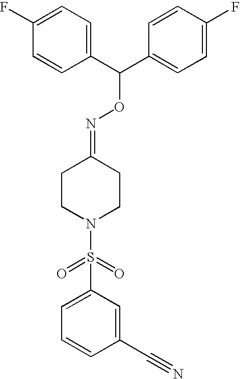

### Example 268

### 1-(3,4-Dimethoxyphenylsulfonyl)piperidin-4-one O-bis(4-fluorophenyl)methyl oxime

was prepared as described in EXAMPLE 50

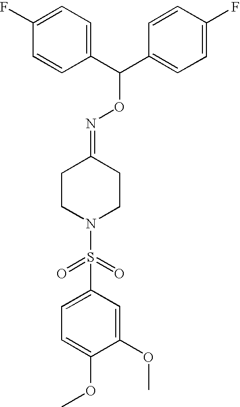

### Example 269

### 3-(Pyridin-4-yl(1-(4-(trifluoromethoxy)phenylsulfonyl)piperidin-4-ylideneaminooxy)methyl)benzonitrile

was prepared as described in Example 107. Starting material was 3-(aminooxy(pyridin-4-yl)methyl)benzonitrile (see example A31).

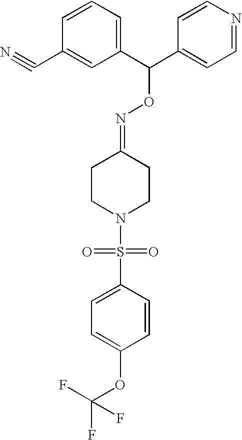

¹H-NMR (DMSO-d₆) δ: 8.62-8.61 (1H, m), 8.47 (1H, d), 7.89 (3H, t), 7.72 (3H, t), 7.63 (2H, d), 7.55 (1H, t), 7.34 (1H, dd), 6.29 (1H, s), 3.15-3.09 (4H, m), 2.79 (2H, t), 2.32 (2H, t).

### Example 270

### 3-(Pyridin-3-yl(1-(4-(trifluoromethoxy)phenylsulfonyl)piperidin-4-ylideneaminooxy)methyl)benzonitrile

was prepared as described in Example 107. Starting material was 3-(aminooxy(pyridin-3-yl)methyl)benzonitrile (see example A32).

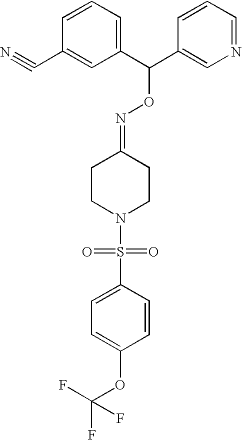

¹H-NMR (DMSO-d₆) δ: 8.51 (2H, m), 7.90 (3H, t), 7.64 (5H, m), 7.38 (2H, d), 6.24 (1H, s), 3.13 (4H, m), 2.81 (2H, t), 2.32 (2H, t).

### Example 271

### 3-(Pyridin-2-yl(1-(4-(trifluoromethoxy)phenylsulfonyl)piperidin-4-ylideneaminooxy)methyl)benzonitrile

was prepared as described in Example 107. Starting material was 3-(aminooxy(pyridin-2-yl)methyl)benzonitrile (see example A33).

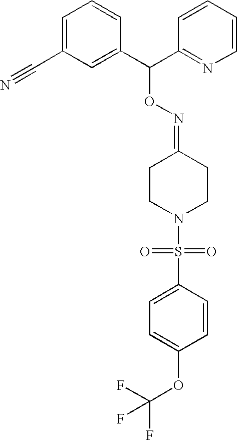

¹H-NMR (DMSO-d₆) δ: 8.47 (1H, d), 7.91 (2H, d), 7.82-7.68 (4H, m), 7.63 (2H, d), 7.53-7.49 (2H, m), 7.27 (1H, dd), 6.19 (1H, s), 3.14-3.11 (4H, m), 2.80 (2H, t), 2.32 (2H, t).

### Example 272

### 3-((1-(6-Chloropyridin-3-ylsulfonyl)piperidin-4-ylideneaminooxy)(cyclopropyl)methyl)benzonitrile

was prepared as described in EXAMPLE 50

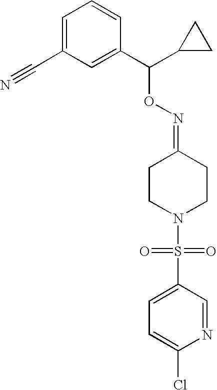

### Example 273

### 3-(2-Methoxy-1-(1-(4-(trifluoromethoxy)phenylsulfonyl)piperidin-4-ylideneaminooxy)ethyl)benzonitrile

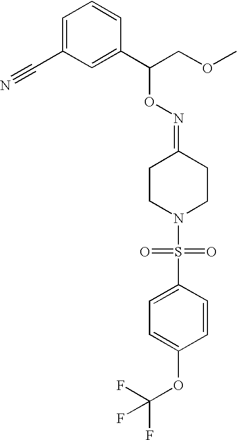

A solution of 3-(2-hydroxy-1-(1-(4-(trifluoromethoxy)phenylsulfonyl)piperidin-4-ylideneaminooxy)ethyl)benzonitrile (80 mg, 0.16 mmol) and iodomethane (0.040 ml, 0.65 mmol) in tetrahydrofuran (4 ml) was added dropwise to a suspension of NaH (60%, 10 mg, 0.24 mmol) in tetrahydrofuran (1 ml) at 0° C. and stirred at room temperature for 2 hours. The reaction was quenched with H₂O (10 ml) and extracted with diethyl ether (20 ml×2). The organic layer was washed with brine, dried (MgSO₄) and evaporated under reduced pressure. The residue was purified by column chromatography on silica (ethyl acetate/hexane, 30/70-50/50) to give the title compound (77 mg, 96%).

### Example 274

### 3-(Cyclopropyl(1-(6-(cyclopropylamino)pyridin-3-ylsulfonyl)piperidin-4-ylideneaminooxy)methyl)benzonitrile

was prepared as described in EXAMPLE 96

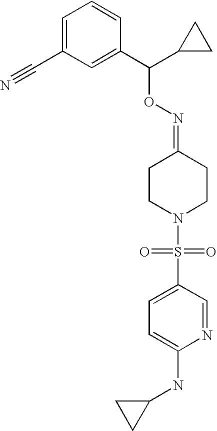

### Example 275

### 3-((1-(6-(Cyclopentylamino)pyridin-3-ylsulfonyl)piperidin-4-ylideneaminooxy)(cyclopropyl)methyl)benzonitrile

was prepared as described in EXAMPLE 96

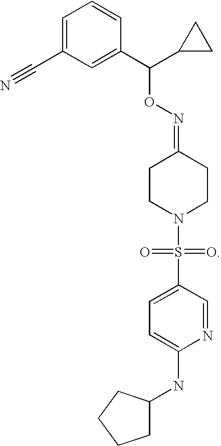

### Example 276

### 1-(3-(Trifluoromethyl)phenylsulfonyl)piperidin-4-one O-1-phenylethyl oxime

was prepared as described in EXAMPLE 65

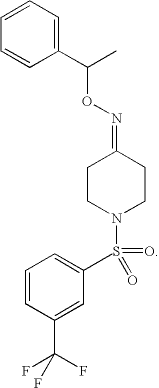

### Example 277

### 1-(3-(Trifluoromethyl)phenylsulfonyl)piperidin-4-one O-cyclohexylmethyl oxime

was prepared as described in EXAMPLE 65

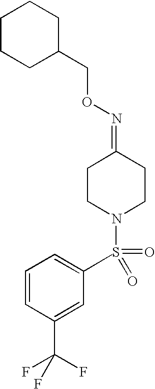

### Example 278

### 5-(4-((3-Cyanophenyl)(cyclopropyl)methoxyimino)piperidin-1-ylsulfonyl)picolinonitrile

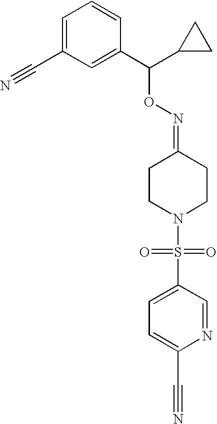

To a solution of 3-((1-(6-Chloropyridin-3-ylsulfonyl)piperidin-4-ylideneaminooxy)(cyclopropyl)methyl)benzonitrile (167 mg, 0.375 mmol) in N,N-dimethylformamide (2 ml), zinc cyanide (33 mg, 0.28 mmol) and tetrakis(triphenylpshosphine)palladium(0) (44 mg, 0.039 mmol) were added and stirred at 100° C. for 7 hours. The reaction was quenched with saturated aqueous K₂CO₃ solution and extracted with diethyl ether (30 ml×2). The organic layer was washed with brine, dried (MgSO₄) and evaporated under reduced pressure. The residue was purified by column chromatography on silica (ethyl acetate/hexane, 30/70-50/50) to give the title compound (97 mg, 60%).

¹H-NMR (DMSO-d₆) δ: 9.08 (1H, d), 8.43 (1H, dd), 8.31 (1H, d), 7.72-7.71 (2H, m), 7.63 (1H, d), 7.53 (1H, t), 4.42 (1H, d), 3.26-3.15 (4H, m), 2.70 (2H, t), 2.27 (2H, t), 1.18-1.11 (1H, m), 0.59-0.38 (4H, m).

### Example 279

### 3-((1-(Benzo[d][1,3]dioxol-5-ylsulfonyl)piperidin-4-ylideneaminooxy)methyl)benzonitrile

was prepared as described in EXAMPLE 50

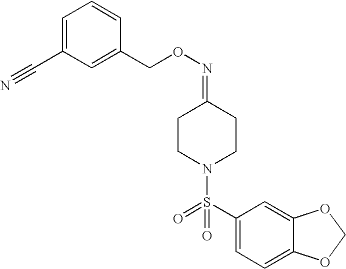

### Example 280

### 3-((1-(4-(Trifluoromethyl)phenylsulfonyl)piperidin-4-ylideneaminooxy)methyl)benzonitrile

was prepared as described in EXAMPLE 50

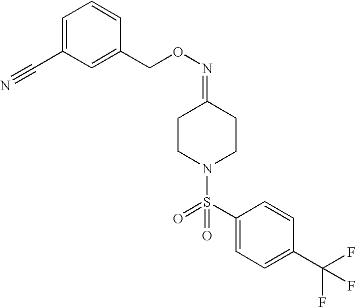

### Example 281

### 3-((1-(4-(2-Fluoroethoxy)phenylsulfonyl)piperidin-4-ylideneaminooxy)methyl)benzonitrile

was prepared as described in EXAMPLE 50

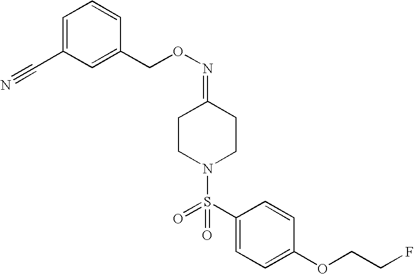

### Example 282

### 3-(4-(3-Cyanobenzyloxyimino)piperidin-1-ylsulfonyl)benzonitrile

was prepared as described in EXAMPLE 50

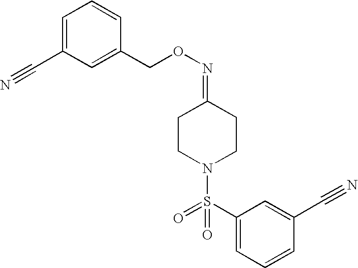

### Example 283

### 3-((1-(Quinolin-8-ylsulfonyl)piperidin-4-ylideneaminooxy)methyl)benzonitrile

was prepared as described in EXAMPLE 50

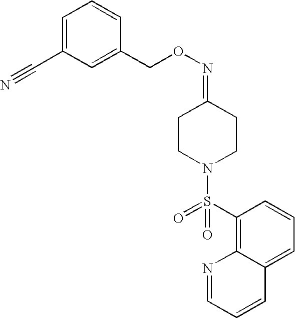

### Example 284

### 3-(4-(3-(Trifluoromethyl)benzyloxyimino)piperidin-1-ylsulfonyl)benzonitrile

was prepared as described in EXAMPLE 50

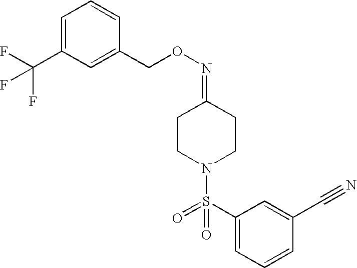

### Example 285

### 1-(3-(Trifluoromethyl)phenylsulfonyl)piperidin-4-one O-bis(4-fluorophenyl)methyl oxime

was prepared as described in EXAMPLE 50

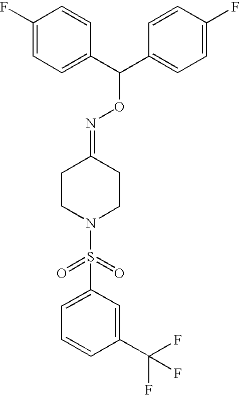

### Example 286

### Methyl 3-(4-(bis(4-fluorophenyl)methoxyimino)piperidin-1-ylsulfonyl)benzoate

was prepared as described in EXAMPLE 50

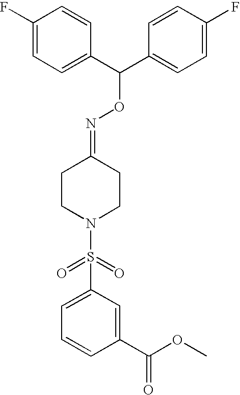

### Example 287

### 1-(Thiophen-3-ylsulfonyl)piperidin-4-one O-bis(4-fluorophenyl)methyl oxime

was prepared as described in EXAMPLE 50

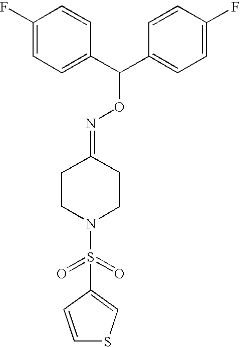

### Example 288

### 1-(Pyridin-2-ylsulfonyl)piperidin-4-one O-bis(4-fluorophenyl)methyl oxime

was prepared as described in EXAMPLE 50

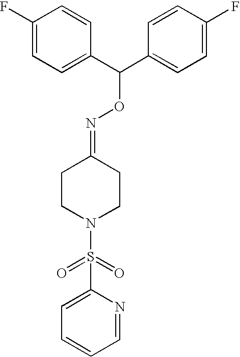

### Example 289

### 1-(2-(Trifluoromethoxy)phenylsulfonyl)piperidin-4-one O-bis(4-fluorophenyl)methyl oxime

was prepared as described in EXAMPLE 50

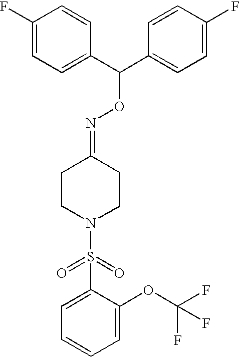

### Example 290

### 1-(3-(Trifluoromethoxy)phenylsulfonyl)piperidin-4-one O-bis(4-fluorophenyl)methyl oxime

was prepared as described in EXAMPLE 50

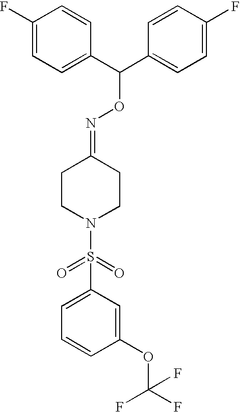

### Example 291

### 1-(Pyridin-3-ylsulfonyl)piperidin-4-one O-bis(4-fluorophenyl)methyl oxime

was prepared as described in EXAMPLE 50

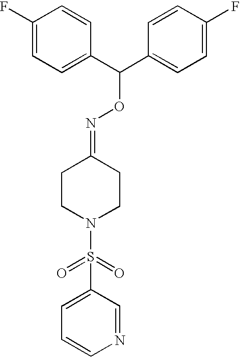

### Example 292

### 1-(3-Chlorophenylsulfonyl)piperidin-4-one O-bis(4-fluorophenyl)methyl oxime

was prepared as described in EXAMPLE 50

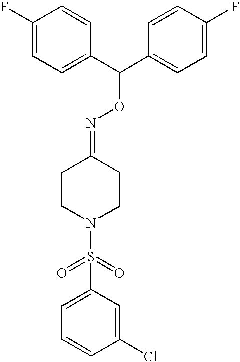

### Example 293

### 3-((1-(4-(2-Cyanoethoxy)phenylsulfonyl)piperidin-4-ylideneaminooxy)methyl)benzonitrile

was prepared as described in EXAMPLE 50

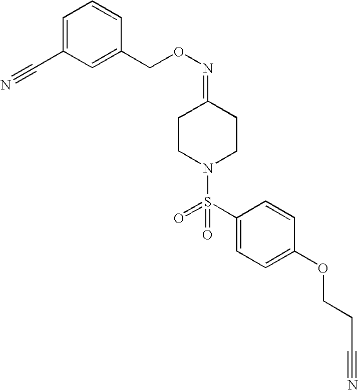

### Example 294

### 1-(3-(Trifluoromethyl)phenylsulfonyl)piperidin-4-one O-2-cyclohexylethyl oxime

was prepared as described in EXAMPLE 65

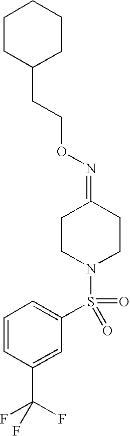

### Example 295

### 1-(4-Fluorobenzyl)-5-((1-(4-(trifluoromethoxy)phenylsulfonyl)piperidin-4-ylideneaminooxy)methyl)pyridin-2(1H)-one

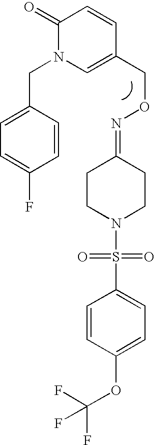

To a solution of 5-[1-(4-Trifluoromethoxybenzenesulfonyl)piperidin-4-ylideneaminooxymethyl]-1H-pyridin-2-one (133 mg, 0.300 mmol), K₂CO₃ (104 mg, 0.750 mmol) and KI (5 mg, 0.03 mmol) in DME (10 ml), 4-fluorobenzyl bromide (0.112 ml, 0.900 mmol) was added and refluxed for 60 hours. The reaction was quenched with H₂O (15 ml) and extracted with ethyl acetate (30 ml×2). The organic layer was washed with brine, dried (MgSO₄) and evaporated under reduced pressure. The residue was purified by column chromatography on silica (ethyl acetate/hexane, 80/20-100/0) to give the title compound (125 mg, 75%).

¹H-NMR (DMSO-d₆) δ: 7.89 (2H, d), 7.82 (1H, d), 7.62 (2H, d), 7.39-7.35 (3H, m), 7.14 (2H, t), 6.38 (1H, d), 5.04 (2H, s), 4.70 (2H, s), 3.12-3.06 (4H, m), 2.55 (2H, t), 2.32 (2H, t).

### Example 296

### 1-(4-Fluorobenzyl)-4-((1-(4-(trifluoromethoxy)phenylsulfonyl)piperidin-4-ylideneaminooxy)methyl)pyridin-2(1H)-one

was prepared as described in EXAMPLE 295

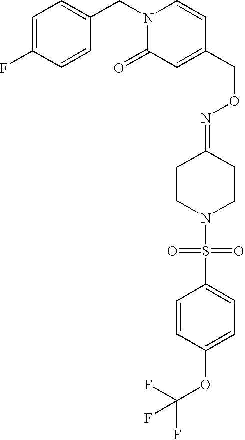

¹H-NMR (DMSO-d₆) δ: 7.91 (2H, d), 7.73 (1H, d), 7.62 (2H, d), 7.35-7.34 (2H, m), 7.17-7.13 (2H, m), 6.26 (1H, s), 6.13 (1H, dd), 5.03 (2H, s), 4.83 (2H, s), 3.15-3.12 (4H, m), 2.65 (2H, t), 2.34 (2H, t).

### Example 297

### 4-(4-((1H-Benzo[d]imidazol-2-yl)methoxyimino)piperidin-1-ylsulfonyl)benzonitrile

was prepared as described in EXAMPLE 50

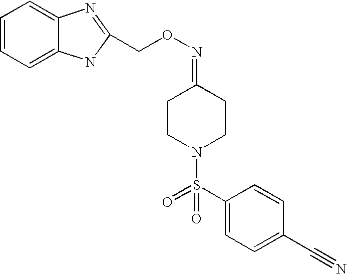

### Example 298

### 1-(3-Chlorophenylsulfonyl)piperidin-4-one O-(1H-benzo[d]imidazol-2-yl)methyl oxime

was prepared as described in EXAMPLE 50

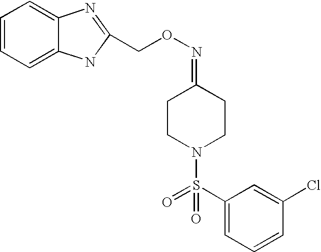

### Example 299

### 3-(4-((1H-Benzo[d]imidazol-2-yl)methoxyimino)piperidin-1-ylsulfonyl)benzonitrile

was prepared as described in EXAMPLE 50

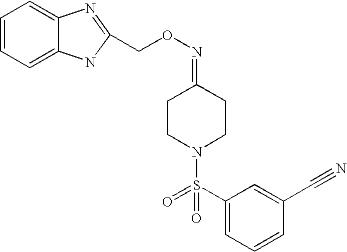

### Example 300

### 1-(Quinolin-8-ylsulfonyl)piperidin-4-one O-(1H-benzo[d]imidazol-2-yl)methyl oxime

was prepared as described in EXAMPLE 50

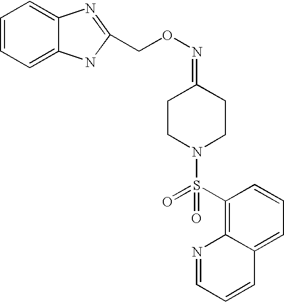

### Example 301

### 1-(4-Fluorophenylsulfonyl)piperidin-4-one O-(1H-benzo[d]imidazol-2-yl)methyl oxime

was prepared as described in EXAMPLE 50

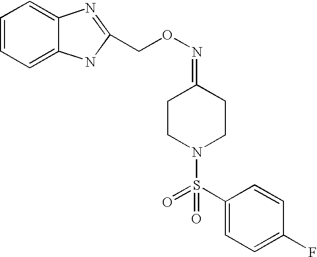

### Example 302

### 3-(2-Morpholino-1-(1-(4-(trifluoromethoxy)phenylsulfonyl)piperidin-4-ylideneaminooxy)ethyl)benzonitrile

was prepared as described in Example 107. Starting material was 3-(1-(aminooxy)-2-morpholinoethyl)benzonitrile (see example A34).

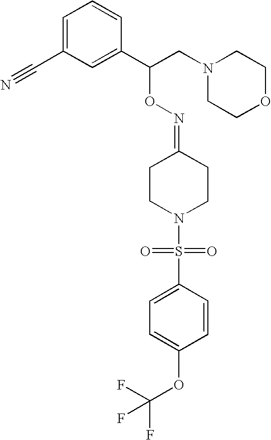

¹H-NMR (DMSO-d₆) δ: 7.90 (2H, d), 7.70 (2H, d), 7.63-7.60 (3H, m), 7.50 (1H, t), 5.23 (1H, t), 3.47 (4H, t), 3.16-3.01 (4H, m), 2.74-2.26 (10H, m).

### Example 303

### 3-(2-Isopropoxy-1-(1-(4-(trifluoromethoxy)phenylsulfonyl)piperidin-4-ylideneaminooxy)ethyl)benzonitrile

was prepared as described in Example 107. Starting material was 3-(1-(aminooxy)-2-isopropoxyethyl)benzonitrile (see example A35).

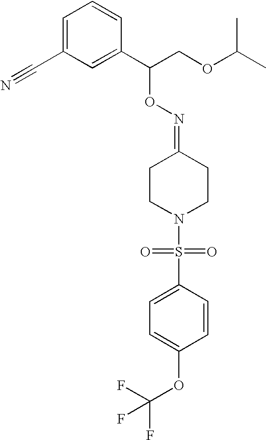

¹H-NMR (DMSO-d₆) δ: 7.91 (2H, d), 7.72-7.71 (2H, m), 7.64-7.62 (3H, m), 7.51 (1H, t), 5.14 (1H, t), 3.67-3.46 (3H, m), 3.11-3.08 (4H, m), 2.69 (2H, t), 2.30 (2H, t), 0.98 (6H, d).

### Example 304

### 3-(2-(4-Fluorophenoxy)-1-(1-(4-(trifluoromethoxy)phenylsulfonyl)piperidin-4-ylideneaminooxy)ethyl)benzonitrile

was prepared as described in Example 107. Starting material was 3-(1-(aminooxy)-2-(4-fluorophenoxy)ethyl)benzonitrile (see example A36).

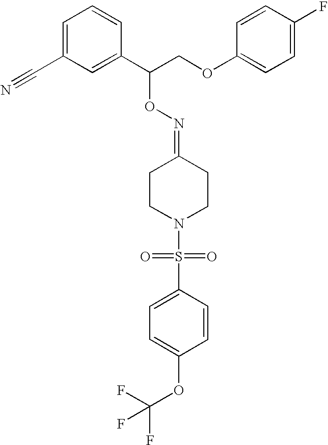

¹H-NMR (DMSO-d₆) δ: 7.90 (2H, d), 7.84 (1H, s), 7.74 (2H, dd), 7.63 (2H, d), 7.55 (1H, t), 7.05 (2H, t), 6.93-6.91 (2H, m), 5.42 (1H, dd), 4.27-4.21 (2H, m), 3.09-3.08 (4H, m), 2.68-2.66 (2H, m), 2.31-2.30 (2H, m).

### Example 305

### 1-(4-(Trifluoromethoxy)phenylsulfonyl)piperidin-4-one O-(3H-imidazo[4,5-b]pyridin-2-yl)methyl oxime

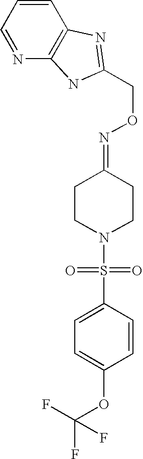

To a solution of tert-butyl 2-((1-(4-(trifluoromethoxy)phenylsulfonyl)piperidin-4-ylideneaminooxy)methyl)-3H-imidazo[4,5-b]pyridine-3-carboxylate (prepared as described in Example 107 starting from tert-butyl 2-(aminooxymethyl)-3H-imidazo[4,5-b]pyridine-3-carboxylate (see example A37), 256 mg, 0.450 mmol) in dichloroethane (4 ml), trifluoroacetic acid (0.70 ml, 9.0 mmol) was added and stirred at room temperature for 1 hour. The reaction mixture was quenched with ammonia solution at 0° C., extracted with chloroform, dried (MgSO₄) and evaporated under reduced pressure. The residue was purified by column chromatography on silica (chloroform/methanol, 98/2-90/10) to give the title compound (110 mg, 52%).

### Example 306

### 1-(4-(Trifluoromethoxy)phenylsulfonyl)piperidin-4-one O-cyclopropyl(4-fluorophenyl)methyl oxime

was prepared as described in Example 107. Starting material was O-(cyclopropyl(4-fluorophenyl)methyl)hydroxylamine (see example A38).

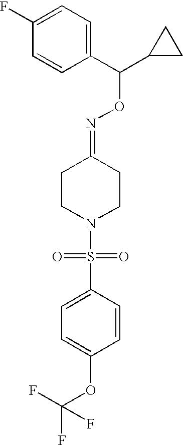

¹H-NMR (DMSO-d₆) δ: 7.90 (2H, d), 7.63 (2H, d), 7.30 (2H, dd), 7.09 (2H, t), 4.38 (1H, d), 3.17-3.11 (2H, m), 3.04-3.01 (2H, m), 2.68-2.66 (2H, m), 2.27 (2H, dd), 1.14-1.09 (1H, m), 0.54 (1H, q), 0.43-0.30 (3H, m).

### Example 307

### 1-(4-(Trifluoromethoxy)phenylsulfonyl)piperidin-4-one O-cyclopropyl(3-(trifluoromethyl)phenyl)methyl oxime

was prepared as described in Example 107. Starting material was O-(cyclopropyl(3-(trifluoromethyl)phenyl)methyl)hydroxylamine (see example A39).

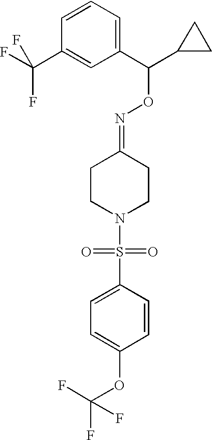

¹H-NMR (DMSO-d₆) δ: 7.90 (2H, d), 7.61-7.53 (6H, m), 4.50 (1H, d), 3.28-3.22 (2H, m), 2.89-2.79 (3H, m), 2.66-2.61 (1H, m), 2.30-2.23 (2H, m), 1.14-1.12 (1H, m), 0.58-0.57 (1H, m), 0.45-0.42 (3.1H, m).

### Example 308

### 1-(4-(Trifluoromethoxy)phenylsulfonyl)piperidin-4-one O-(6-chloro-3H-imidazo[4,5-b]pyridin-2-yl)methyl oxime

was prepared as described in Example 305. Starting material was tert-butyl 2-(aminooxymethyl)-6-chloro-3H-imidazo[4,5-b]pyridine-3-carboxylate (see example A40).

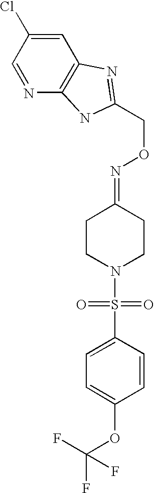

¹H-NMR (DMSO-d₆) δ: 8.32 (1H, d), 8.06 (1H, s), 7.91 (2H, d), 7.63 (2H, d), 5.19 (2H, s), 3.13 (4H, t), 2.69 (2H, t), 2.34 (2H, t).

### Example 309

### 1-(3-(Trifluoromethyl)phenylsulfonyl)piperidin-4-one O-cyclohexyl oxime

was prepared as described in EXAMPLE 65

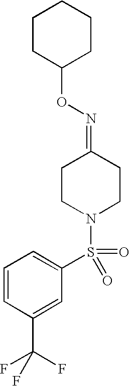

### Example 310

### 1-(3-(Trifluoromethyl)phenylsulfonyl)piperidin-4-one O-phenethyl oxime

was prepared as described in EXAMPLE 65

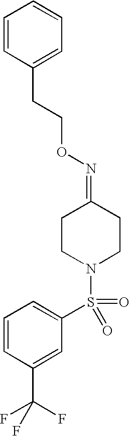

### Example 311

### 1-(4-(Trifluoromethoxy)phenylsulfonyl)piperidin-4-one O-biphenyl-2-ylmethyl oxime

was prepared as described in Example 107.

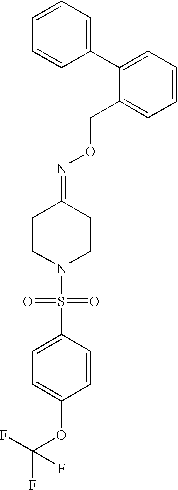

¹H-NMR (CDCl₃) δ: 7.81 (2H, dd), 7.46-7.28 (11H, m), 4.97 (2H, s), 3.19 (2H, t), 3.11 (2H, t), 2.65 (2H, t), 2.40 (2H, t).

### Example 312

### 1-(4-(Trifluoromethoxy)phenylsulfonyl)piperidin-4-one O-biphenyl-3-ylmethyl oxime

was prepared as described in Example 107.

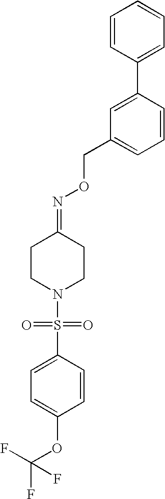

### Example 313

### 1-(4-(Trifluoromethoxy)phenylsulfonyl)piperidin-4-one O-(3-phenylpyridin-2-yl)methyl oxime

was prepared as described in Example 107. Starting material was O-((3-phenylpyridin-2-yl)methyl)hydroxylamine (see example A41).

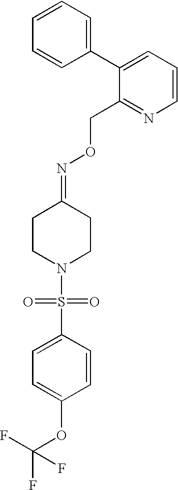

¹H-NMR (CDCl₃) δ: 8.62 (1H, d), 7.81 (2H, d), 7.63 (1H, d), 7.35-7.33 (8H, m), 5.12 (2H, s), 3.17 (2H, t), 3.09 (2H, t), 2.59 (2H, t), 2.38 (2H, t).

### Example 314

### 1-(4-Fluorophenyl)-5-((1-(4-(trifluoromethoxy)phenylsulfonyl)piperidin-4-ylideneaminooxy)methyl)pyridin-2(1H)-one

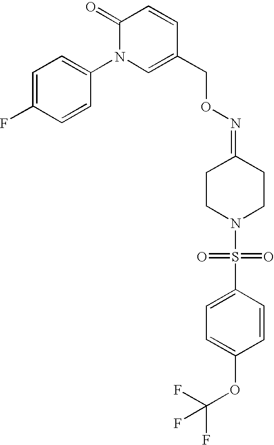

To a suspension of 5-[1-(4-Trifluoromethoxybenzenesulfonyl)piperidin-4-ylideneaminooxymethyl]-1H-pyridin-2-one (223 mg, 0.500 mmol), K₂CO₃ (138 mg, 1.00 mmol) and CuI (19 mg, 0.10 mmol) in toluene (2 ml), a solution of p-bromofluorobenzene (0.060 ml, 0.600 mmol) and (1S,2S)-(+)-N,N′-dimethylcyclohexane 1,2-diamine (14 mg, 0.100 mmol) was added and refluxed for 19 hours. The solvent was evaporated under reduced pressure and the residue was purified by column chromatography on silica (ethyl acetate/hexane, 50/50-70/30) to give the title compound (241 mg, 89%).

¹H-NMR (DMSO-d₆) δ: 7.90 (2H, d), 7.67-7.62 (3H, m), 7.49-7.46 (3H, m), 7.33 (2H, t), 6.47 (1H, d), 4.75 (2H, s), 3.15-3.08 (4H, m), 2.58 (2H, t), 2.36 (2H, t).

### Example 315

### 4-(2-oxo-5-((1-(4-(trifluoromethoxy)phenylsulfonyl)piperidin-4-ylideneaminooxy)methyl)pyridin-1(2H)-yl)benzonitrile

was prepared as described in Example 314.

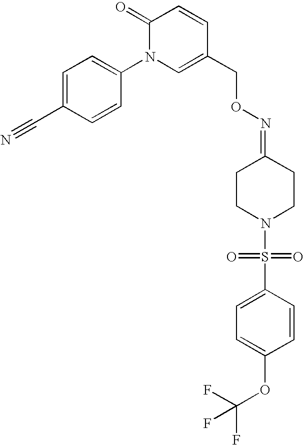

¹H-NMR (DMSO-d₆) δ: 8.00 (2H, d), 7.90 (2H, d), 7.73 (1H, d), 7.65-7.62 (4H, m), 7.53 (1H, dd), 6.50 (1H, d), 4.76 (2H, s), 3.15-3.08 (4H, m), 2.58 (2H, t), 2.35 (2H, t).

### Example 316

### 1-(4-(4-(3-(Trifluoromethyl)benzyloxyimino)piperidin-1-ylsulfonyl)phenyl)ethanone

was prepared as described in EXAMPLE 50

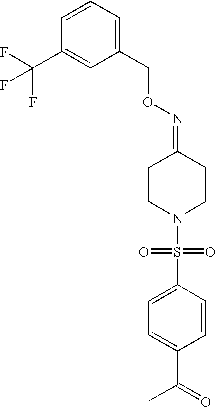

### Example 317

### 3-((1-(4-Acetylphenylsulfonyl)piperidin-4-ylideneaminooxy)methyl)benzonitrile

was prepared as described in EXAMPLE 50

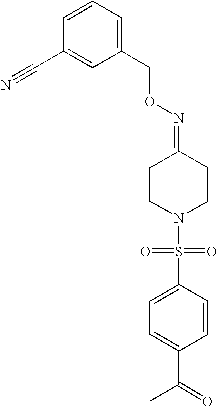

### Example 318

### 1-(4-(4-(4-Chlorobenzyloxyimino)piperidin-1-ylsulfonyl)phenyl)ethanone

was prepared as described in EXAMPLE 50

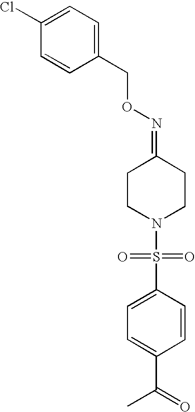

### Example 319

### 1-(4-(Trifluoromethoxy)phenylsulfonyl)piperidin-4-one O-3-(trifluoromethyl)phenyl oxime

was prepared as described in Example 107.

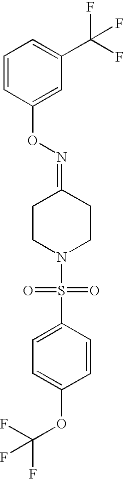

### Example 320

### 1-(6-benzylaminopyridine-3-sulfonyl)piperidin-4-one O-(3-trifluoromethylbenzyl)oxime

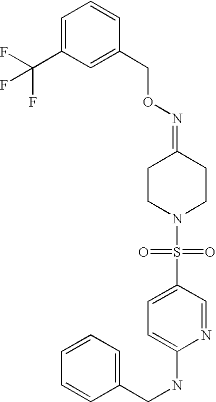

To a solution of 1-(6-chloropyridine-3-sulfonyl)piperidin-4-one O-(3-trifluoromethylbenzyl)oxime (134 mg, 0.300 mmol) in tetrahydrofuran (3 ml), benzylamine (161 mg, 1.50 mmol) was added and stirred at 80° C. for 40 hours. The solvent was evaporated under reduced pressure and the residue was diluted with ethyl acetate and washed with H₂O and brine, dried (MgSO4) and evaporated under reduced pressure. The residue was purified by column chromatography on silica (ethyl acetate/hexane 25/75-50/50) to give the title compound (151 mg, 97%).

### Example 321

### 1-(6-Cyclopropylaminopyridine-3-sulfonyl)piperidin-4-one O-(3-trifluoromethylbenzyl)oxime

was prepared as described in EXAMPLE 320

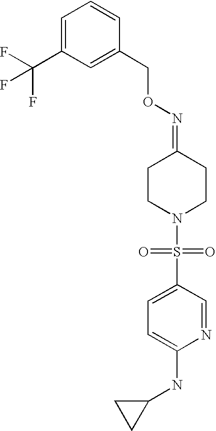

¹H-NMR (DMSO-d₆) δ: 8.30 (1H, d), 7.80 (1H, d), 7.68-7.59 (5H, m), 6.67 (1H, t), 5.09 (2H, s), 3.04-3.00 (4H, m), 2.64-2.63 (3H, m), 2.33 (2H, t), 0.76-0.74 (2H, m), 0.49-0.46 (2H, m).

### Example 322

### 1-(6-Phenylaminopyridine-3-sulfonyl)piperidin-4-one O-(3-trifluoromethylbenzyl)oxime

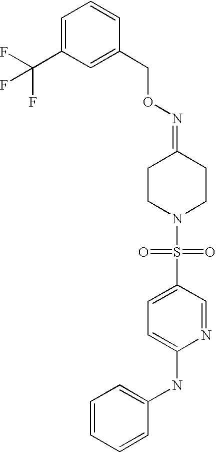

A solution of 1-(6-chloropyridine-3-sulfonyl)piperidin-4-one O-(3-trifluoromethylbenzyl)oxime (134 mg, 0.300 mmol), aniline (0.027 ml, 0.300 mmol), palladium (II) acetate (2.0 mg, 0.009 mmol), 4,5-bis(diphenylphosphino)-9,9-dimethylxanthene (10 mg, 0.018 mmol) and sodium tert-butoxide (40 mg, 0.42 mmol) in dioxane (3 ml) was stirred at 100° C. for 30 minutes. The solvent was evaporated under reduced pressure and the residue was diluted with ethyl acetate and washed with H₂O and brine, dried (MgSO₄) and evaporated under reduced pressure. The residue was purified by column chromatography on silica (ethyl acetate/hexane 20/80-100/0) to give the title compound (43 mg, 28%).

### Example 323

### 6-[1-(4-Butylbenzenesulfonyl)piperidin-4-ylideneaminooxymethyl]-1H-pyridin-2-one

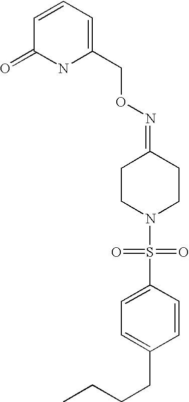

To a solution of 1-(4-butylbenzenesulfonyl)piperidin-4-one O-(6-methoxypyridin-2-ylmethyl)oxime (60 mg, 0.139 mmol) and KI (115 mg, 0.695 mmol) in acetonitrile (2 ml), chlorotrimethylsilane (76 mg, 0.695 mmol) was added and stirred at room temperature for 5 hours and at 65° C. for 1 hour. 10% aqueous Na₂S₂O₃ solution (20 ml) was added, extracted with ethyl acetate (30 ml×2), washed with brine, dried (MgSO₄) and evaporated under reduced pressure. The residue was purified by column chromatography on silica (ethyl acetate/methanol, 100/0-90/10) to afford the title compound (50 mg, 86%).

### Example 324

### 1-[2-(2-Methoxy-ethoxy)benzenesulfonyl]piperidin-4-one O-(3-trifluoromethylbenzyl)oxime

was prepared as described in EXAMPLE 91

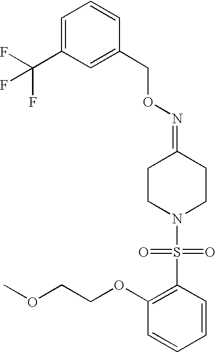

### Example 325

### 2-(Piperidin-1-yl)-1-(4-(3-(trifluoromethyl)benzyloxyimino)piperidin-1-yl)ethanone

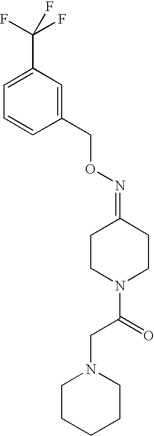

a) Chloroacetyl chloride (0.55 g, 5.0 mmol) was added to a mixture of piperidin-4-one O-3-(trifluoromethyl)benzyl oxime (1.0 g, 3.6 mmol) and triethylamine in dichloromethane (20 ml) at 0° C. The reaction mixture was warmed to room temperature over 6 h, washed with H₂O (10 ml) and evaporated under reduced pressure. The residue was purified by column chromatography on silica to afford 2-chloro-1-(4-(3-(trifluoromethyl)benzyloxyimino)piperidin-1-yl)ethanone (1.0 g, 70%).

b) A mixture of 2-chloro-1-(4-(3-(trifluoromethyl)benzyloxyimino)piperidin-1-yl)ethanone (0.25 g, 0.70 mmol), K₂CO₃ (1.0 g, 7.8 mmol), KI (20 mg) and piperidine (0.07 ml, 0.8 mmol) in acetonitrile (4 ml) was stirred at 40° C. for 48 hours. After cooling to room temperature, the reaction mixture was diluted with ethyl acetate (10 ml), washed with H₂O and evaporated under reduced pressure. The residue was purified by column chromatography on silica (ethyl acetate/hexane, 30/70) to afford the title compound (100 mg, 36%).

¹H-NMR (CDCl₃) δ: 7.45-7.60 (4H, m), 5.12 (2H, d), 3.64-3.72 (4H, m), 3.16 (2H, s), 2.69-2.73 (1H, m), 2.61-2.64 (1H, m), 2.33-2.44 (6H, m), 1.54-1.59 (4H, m), 1.40-1.45 (2H, m).

### Example 326

### 2-(2,2,2-Trifluoroethylamino)-1-(4-(3-(trifluoromethyl)benzyloxyimino)piperidin-1-yl)ethanone

was prepared as described in EXAMPLE 325

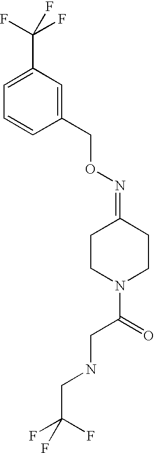

### Example 327

### N-(3,5-Bis(trifluoromethyl)phenyl)-2-(4-(3-(trifluoromethyl)benzyloxyimino)piperidin-1-yl)acetamide

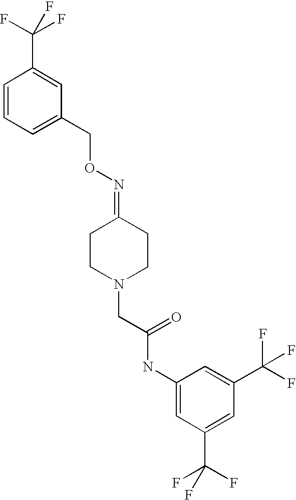

a) Chloroacetyl chloride (1.2 g) was added to a solution of 3,5-bis(trifluoromethyl)aniline (2.0 g) and triethylamine (2 ml) in dichloromethane (20 ml) at at 0° C. The reaction mixture was warmed to room temperature over 3 hours, quenched with H₂O (6 ml), washed with brine (4 ml) and evaporated under reduced pressure. The residue was purified by column chromatography on silica (ethyl acetate/hexane, 10/90) to afford N-(3,5-bis(trifluoromethyl)phenyl)-2-chloroacetamide as colorless oil.

b) A mixture of N-(3,5-bis(trifluoromethyl)phenyl)-2-chloroacetamide (150 mg), piperidin-4-one O-3-(trifluoromethyl)benzyl oxime (120 mg), K₂CO₃ (300 mg), and KI (20 mg) in acetonitrile (4 ml) was stirred at 40° C. for 72 hours. The reaction mixture was diluted with ethyl acetate (4 ml), washed with H₂O (2 ml), brine (4 ml) and evaporated under reduced pressure. The residue was purified by column chromatography on silica (ethyl acetate/hexane, 30/70) to afford the title compound (140 mg, 50%).

¹H-NMR (CDCl₃) δ: 9.40 (1H, br), 8.08 (2H, s), 7.45-7.65 (5H, m), 5.12 (2H, s), 3.64-3.72 (4H, m), 3.24 (2H, s), 2.71-2.83 (6H, m), 2.44-2.49 (2H, m).

### Example 328

### 2-(4-(Bis(4-fluorophenyl)methoxyimino)piperidin-1-yl)-N-(3,5-bis(trifluoromethyl)phenyl)acetamide

was prepared as described in EXAMPLE 327

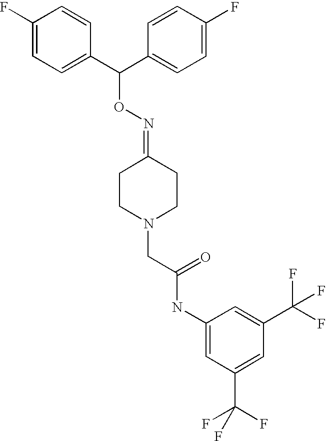

### Example 329

### N-(3,5-Bis(trifluoromethyl)benzyl)-2-(4-(3-(trifluoromethyl)benzyloxyimino)piperidin-1-yl)acetamide

was prepared as described in EXAMPLE 327

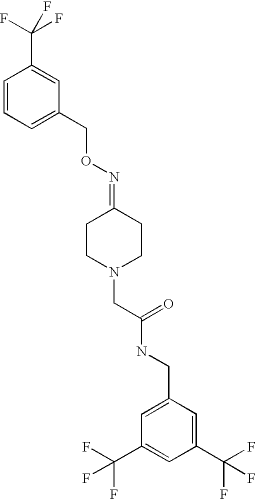

### Example 330

### 2-(4-(Bis(4-fluorophenyl)methoxyimino)piperidin-1-yl)-N-(3,5-bis(trifluoromethyl)benzyl)acetamide

was prepared as described in EXAMPLE 327

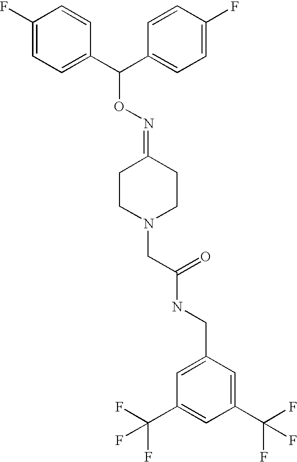

### Example 331

### N-(2-Ethoxy-4-fluorophenyl)-2-(4-(3-(trifluoromethyl)benzyloxyimino)piperidin-1-yl)acetamide

was prepared as described in EXAMPLE 327

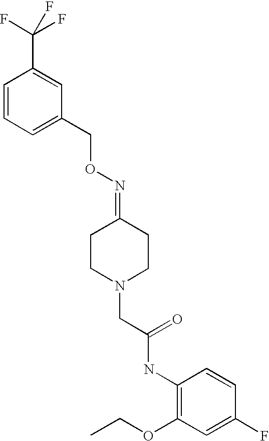

### Example 332

### 2-(4-(Bis(4-fluorophenyl)methoxyimino)piperidin-1-yl)-N-(2-ethoxy-4-fluorophenyl)acetamide

was prepared as described in EXAMPLE 327

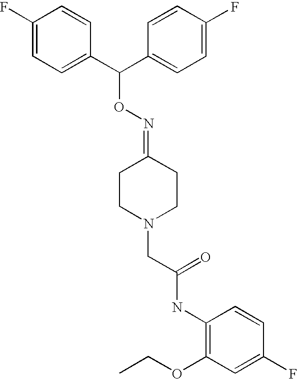

### Example 333

### 1-(4-Butylphenylsulfonyl)piperidin-4-one O-4-chloro-2-fluorobenzyl oxime

was prepared as described in EXAMPLE 65

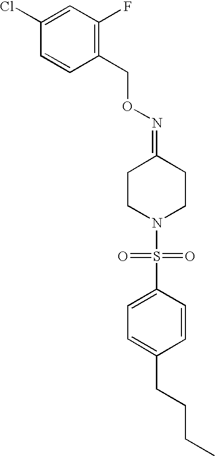

### Example 334

### 2-((1-(3-Chlorophenylsulfonyl)piperidin-4-ylideneaminooxy)methyl)isonicotinonitrile

was prepared as described in Example 107.

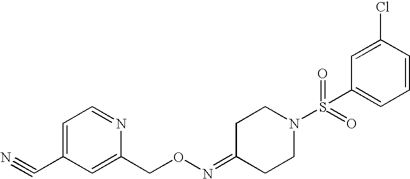

### Example 335

### 2-((1-(Pyridin-3-ylsulfonyl)piperidin-4-ylideneaminooxy)methyl)isonicotinonitrile

was prepared as described in Example 107.

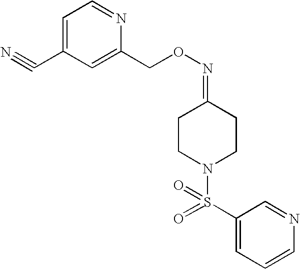

### Example 336

### 1-(3-Fluorophenylsulfonyl)piperidin-4-one O-bis(4-fluorophenyl)methyl oxime

was prepared as described in Example 107.

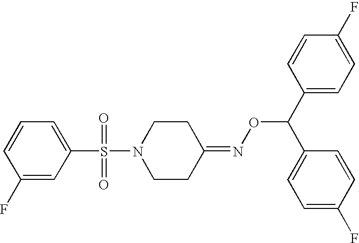

### Example 337

### 3-(4-(Bis(4-fluorophenyl)methoxyimino)piperidin-1-ylsulfonyl)-N-methoxy-N-methylbenzamide

was prepared as described in Example 107.

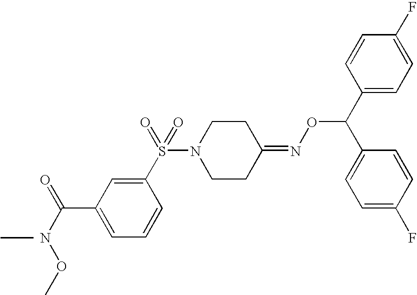

### Example 338

### 3-(4-(Bis(4-fluorophenyl)methoxyimino)piperidin-1-ylsulfonyl)-N,N-dimethylbenzamide

was prepared as described in Example 107.

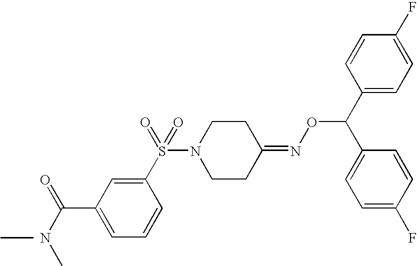

### Example 339

### 1-(Pyridin-3-ylsulfonyl)piperidin-4-one O-4-chlorobenzyl oxime

was prepared as described in Example 107.

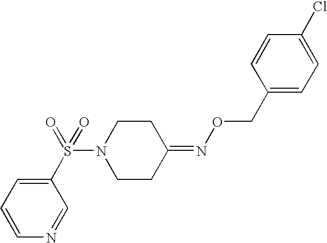

### Example 340

### 2-((1-(3-(Trifluoromethyl)phenylsulfonyl)piperidin-4-ylideneaminooxy)methyl)isonicotinonitrile

was prepared as described in Example 107.

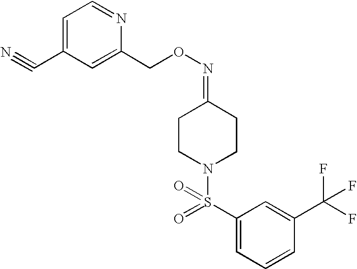

### Example 341

### 2-((1-(3-(Trifluoromethoxy)phenylsulfonyl)piperidin-4-ylideneaminooxy)methyl)isonicotinonitrile

was prepared as described in Example 107.

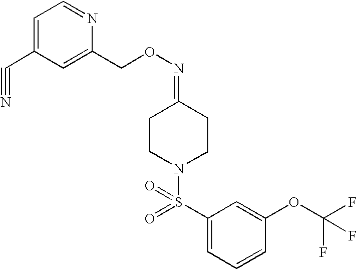

### Example 342

### 1-(Pyridin-2-ylsulfonyl)piperidin-4-one O-4-chlorobenzyl oxime

was prepared as described in Example 107.

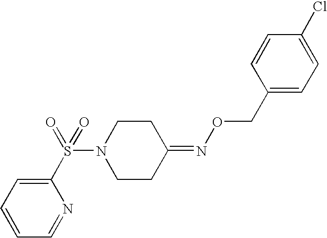

### Example 343

### 1-(3-Chlorophenylsulfonyl)piperidin-4-one O-4-chlorobenzyl oxime

was prepared as described in Example 107.

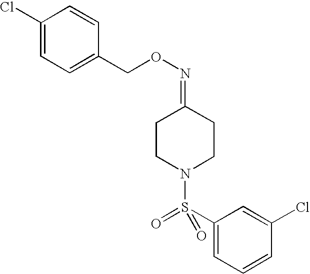

### Example 344

### 1-(Quinolin-8-ylsulfonyl)piperidin-4-one O-4-chlorobenzyl oxime

was prepared as described in Example 107.

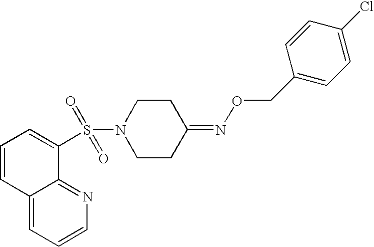

### Example 345

### 2-((1-(4-Chlorophenylsulfonyl)piperidin-4-ylideneaminooxy)methyl)isonicotinonitrile

was prepared as described in Example 107.

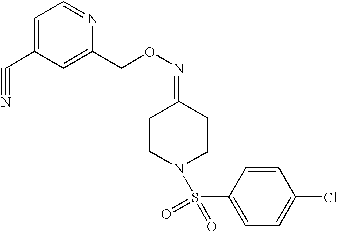

### Example 346

### 1-(4-Fluorophenylsulfonyl)piperidin-4-one O-4-chlorobenzyl oxime

was prepared as described in Example 107.

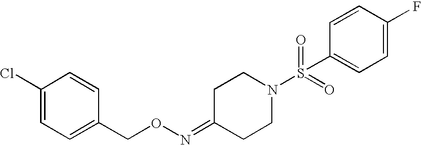

### Example 347

### 1-(3-Fluorophenylsulfonyl)piperidin-4-one O-4-chlorobenzyl oxime

was prepared as described in Example 107.

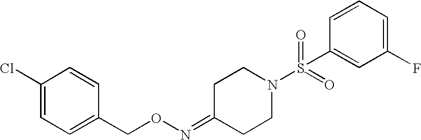

### Example 348

### 2-((1-(4-Cyanophenylsulfonyl)piperidin-4-ylideneaminooxy)methyl)isonicotinonitrile

was prepared as described in Example 107.

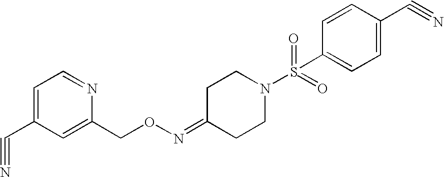

### Example 349

### 2-((1-(3-Cyanophenylsulfonyl)piperidin-4-ylideneaminooxy)methyl)isonicotinonitrile

was prepared as described in Example 107.

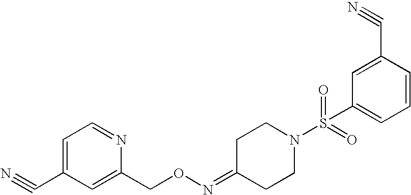

### Example 350

### 1-(4-Chlorophenylsulfonyl)piperidin-4-one O-bis(4-fluorophenyl)methyl oxime

was prepared as described in Example 107.

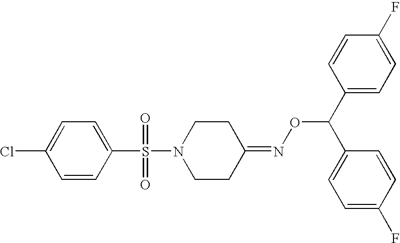

### Example 351

### 1-(4-Chlorophenylsulfonyl)piperidin-4-one O-4-chlorobenzyl oxime

was prepared as described in Example 107.

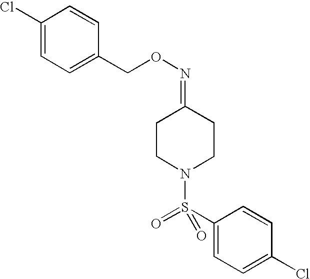

### Example 352

### 2-((1-(4-Fluorophenylsulfonyl)piperidin-4-ylideneaminooxymethyl)isonicotinonitrile

was prepared as described in Example 107.

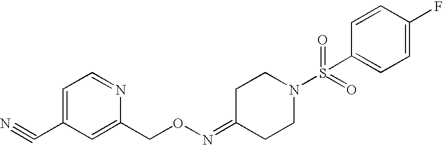

### Example 353

### 2-((1-(3-Fluorophenylsulfonyl)piperidin-4-ylideneaminooxy)methyl)isonicotinonitrile

was prepared as described in Example 107.

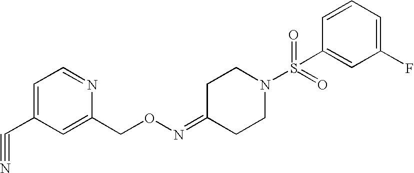

### Example 354

### 2-((1-(2-(Trifluoromethoxy)phenylsulfonyl)piperidin-4-ylideneaminooxy)methyl)isonicotinonitrile

was prepared as described in Example 107.

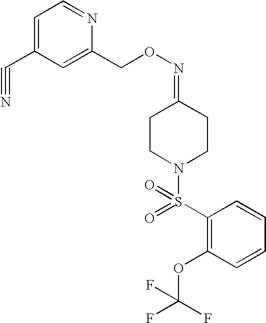

### Example 355

### 2-((1-(Quinolin-8-ylsulfonyl)piperidin-4-ylideneaminooxy)methyl)isonicotinonitrile

was prepared as described in Example 107.

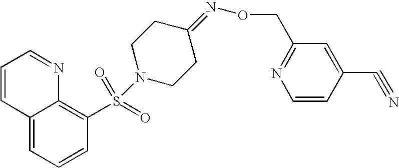

### Example 356

### 2-((1-(Pyridin-2-ylsulfonyl)piperidin-4-ylideneaminooxy)methyl)isonicotinonitrile

was prepared as described in Example 107.

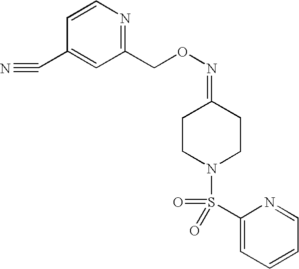

### Example 357

### 1-(4-(Trifluoromethoxy)phenylsulfonyl)piperidin-4-one O-(6-chloropyridin-3-yl)methyl oxime

was prepared as described in Example 107.

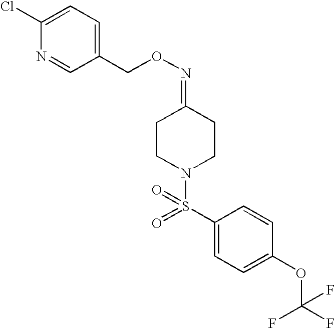

### Example 358

### 1-(4-(Trifluoromethoxy)phenylsulfonyl)piperidin-4-one O-(2-chloropyridin-3-yl)methyl oxime

was prepared as described in Example 107. Starting material was O-((2-chloropyridin-3-yl)methyl)hydroxylamine (see example A42).

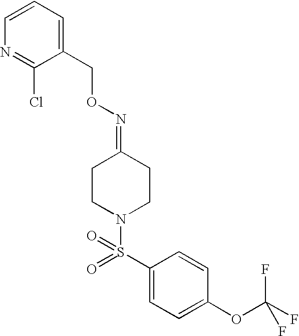

### Example 359

### 1-(3-(Trifluoromethyl)phenylsulfonyl)piperidin-4-one O-4-chlorobenzyl oxime

was prepared as described in Example 107.

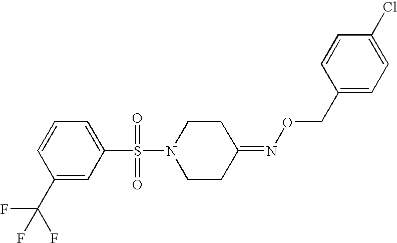

### Example 360

### 1-(4-(Trifluoromethoxy)phenylsulfonyl)piperidin-4-one O-propyl oxime

was prepared as described in EXAMPLE 65

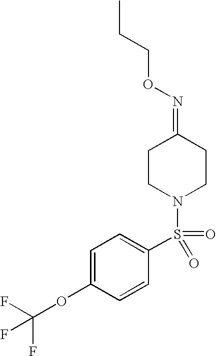

### Example 361

### 1-(4-(Trifluoromethoxy)phenylsulfonyl)piperidin-4-one O-isobutyl oxime

was prepared as described in EXAMPLE 65

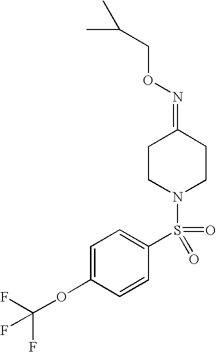

### Example 362

### 1-(3-(Trifluoromethyl)phenylsulfonyl)piperidin-4-one O-isopropyl oxime

was prepared as described in EXAMPLE 65

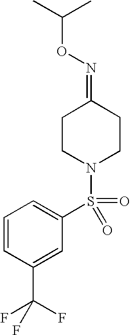

### Example 363

### 1-(3-(Trifluoromethyl)phenylsulfonyl)piperidin-4-one O-isobutyl oxime

was prepared as described in EXAMPLE 65

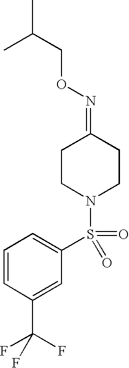

### Example 364

### 1-(3-(Trifluoromethyl)phenylsulfonyl)piperidin-4-one O-3-methylbut-2-enyl oxime

was prepared as described in EXAMPLE 65

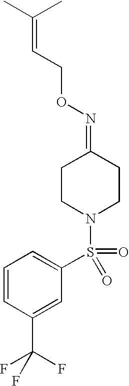

### Example 365

### 1-(3-(Trifluoromethyl)phenylsulfonyl)piperidin-4-one O-propyl oxime

was prepared as described in EXAMPLE 65

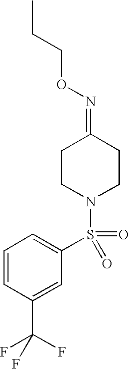

### Example 366

### 1-(3-(Trifluoromethyl)phenylsulfonyl)piperidin-4-one O-isopentyl oxime

was prepared as described in EXAMPLE 65

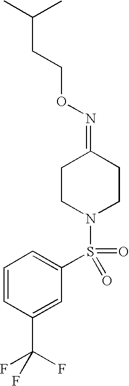

¹H-NMR (CDCl₃) δ: 8.03 (1H, s), 7.96 (1H, d), 7.87 (1H, d), 7.70 (1H, dd), 4.0 (2H, t), 3.24 (2H, t), 3.17 (2H, t), 2.7 (2H, t), 2.45 (2H, dd), 1.60-1.68 (1H, m), 1.46-1.51 (2H, m), 0.89 (6H, d).

### Example 367

### 1-(3-(Trifluoromethyl)phenylsulfonyl)piperidin-4-one O-hexan-2-yl oxime

was prepared as described in EXAMPLE 65

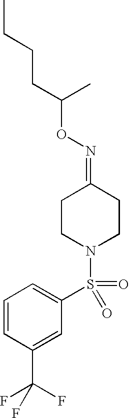

¹H-NMR (CDCl₃) δ: 8.03 (1H, s), 7.96 (1H, d), 7.87 (1H, d), 7.70 (1H, dd), 4.04-4.09 (1H, m), 3.24 (2H, t), 3.17 (2H, t), 2.70 (2H, t), 2.45 (2H, dd), 1.20-1.60 (6H, m), 1.15 (3H, d), 0.87-0.90 (3H, m).

### Example 368

### 1-(3-(Trifluoromethyl)phenylsulfonyl)piperidin-4-one O-cinnamyl oxime

was prepared as described in EXAMPLE 65

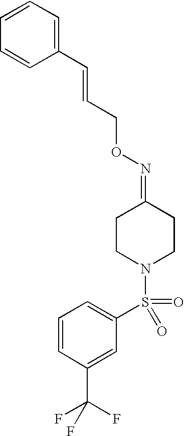

¹H-NMR (CDCl₃) δ: 8.03 (1H, s), 7.96 (1H, d), 7.87 (1H, d), 7.70 (1H, dd), 7.28-7.38 (5H, m), 6.58 (1H, d), 6.30 (1H, dt), 4.64 (2H, dd), 3.25 (2H, t), 3.18 (2H, t), 2.74 (2H, dd), 2.47 (2H, dd).

### Example 369

### 1-(3-(Trifluoromethyl)phenylsulfonyl)piperidin-4-one O-heptan-2-yl oxime

was prepared as described in EXAMPLE 65

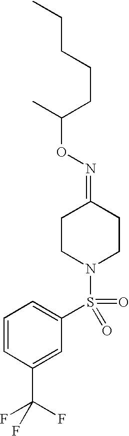

¹H-NMR (CDCl₃) δ: 8.03 (1H, s), 7.96 (1H, d), 7.87 (1H, d), 7.70 (1H, dd), 4.04-4.09 (1H, m), 3.23 (2H, t), 3.16 (2H, t), 2.70 (2H, t), 2.45 (2H, t), 1.20-1.60 (8H, m), 1.14 (3H, d), 0.84-0.87 (3H, m).

### Example 370

### 3-(4-(Bis(4-fluorophenyl)methoxyimino)piperidin-1-ylsulfonyl)-N-methylbenzamide

was prepared as described in Example 107.

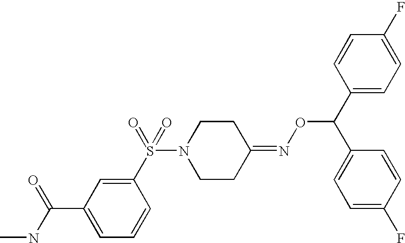

### Example 371

### 3-(4-(Bis(4-fluorophenyl)methoxyimino)piperidin-1-ylsulfonyl)benzamide

was prepared as described in Example 107.

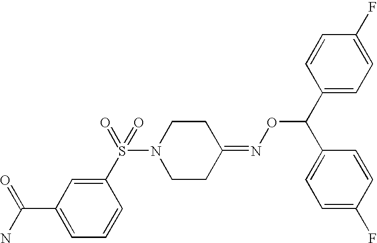

### Example 372

### 1-(4-(Trifluoromethoxy)phenylsulfonyl)piperidin-4-one O-(3-chloropyridin-4-yl)methyl oxime

was prepared as described in Example 107. Starting material was O-((3-chloropyridin-4-yl)methyl)hydroxylamine (see example A43).

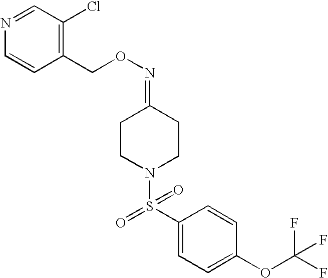

### Example 373

### 1-(4-(Ttrifluoromethoxy)phenylsulfonyl)piperidin-4-one O-(6-chloropyridin-2-yl)methyl oxime

was prepared as described in Example 107. Starting material was O-((6-Chloropyridin-2-yl)methyl)hydroxylamine (see example A44).

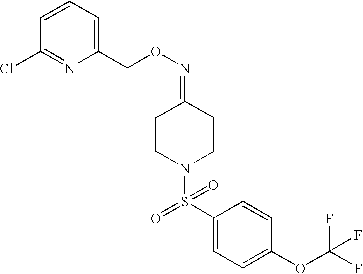

### Example 374

### 1-(4-(Trifluoromethoxy)phenylsulfonyl)piperidin-4-one O-(4-chloropyridin-3-yl)methyl oxime

was prepared as described in Example 107. Starting material was O-((4-Chloropyridin-3-yl)methyl)hydroxylamine (see example A45).

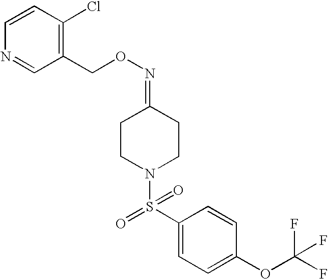

### Example 375

### 1-(4-(Trifluoromethoxy)phenylsulfonyl)piperidin-4-one O-(2-chloropyridin-4-yl)methyl oxime

was prepared as described in Example 107. Starting material was O-((2-Chloropyridin-4-yl)methyl)hydroxylamine (see example A46).

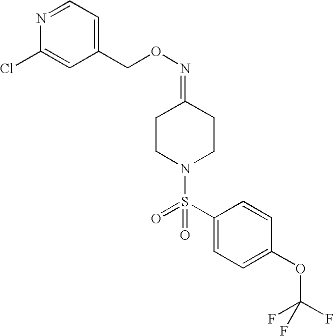

### Example 376

### 1-(4-(Trifluoromethoxy)phenylsulfonyl)piperidin-4-one O-(3-chloropyridin-2-yl)methyl oxime

was prepared as described in Example 107. Starting material was O-((3-Chloropyridin-2-yl)methyl)hydroxylamine (see example A47).

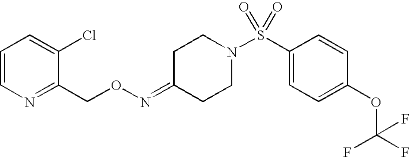

### Example 377

### 5-((1-(4-(Trifluoromethoxy)phenylsulfonyl)piperidin-4-ylideneaminooxy)methyl)nicotinonitrile

was prepared as described in Example 107. Starting material was 5-(Aminooxymethyl)nicotinonitrile (see example A48).

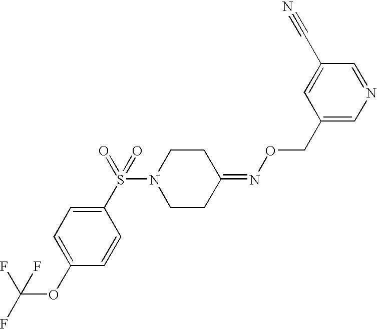

Purity of compounds was verified by LCMS measurement. LCMS method is as follows;

Method A

Column: Phenomemex Luna C18 (4.6×50 mm, 5 micron particle size), Temperature: 50° C., Monitored at OD 254 nm, reference 360 nm, Flow rate: 2 ml/min.

HPLC Gradient (Buffer A=0.1% formic acid/H₂O, Buffer B=0.1% formic acid/acetonitrile)

TABLE 2

| Time (min.) | % B |
|---|---|
| 0 | 15 |
| 1.9 | 45 |
| 4.3 | 45 |
| 8.3 | 95 |
| 11.3 | 95 |
| 11.4 | 15 |
| 15.4 | 15 |

Method B

Column: Discovery HS C18 (4.6×150 mm, 3 micron particle size), Temperature: 25° C., Monitored at OD 260 nm, reference 360 nm, Flow rate: 1 ml/min.

HPLC Gradient (Buffer A=0.1% trifluoroacetic acid/H₂O, Buffer B=0.1% trifluoroacetic acid/acetonitrile)

TABLE 3

| Time (min.) | % B |
|---|---|
| 0 | 15 |
| 1.9 | 45 |
| 4.3 | 45 |
| 8.3 | 95 |
| 11.3 | 95 |
| 11.4 | 15 |
| 15.4 | 15 |

Method C

Column: Sunfire-C18 (2.1×50 mm, 3.5 micron particle size), Temperature: 50° C., Monitored at OD 214 nm, reference 360 nm, Flow rate: 0.9 ml/min.

HPLC Gradient (Buffer A=0.05% trifluoroacetic acid/H₂O, Buffer B=0.05% trifluoroacetic acid/acetonitrile)

TABLE 4

| Time (min.) | % B |
|---|---|
| 0 | 10 |
| 2.2 | 90 |
| 4.0 | 90 |

TABLE 5
LCMS data and properties of the example compounds

| EXAMPLE | LCMS | Retention | Purity | | |
|---|---|---|---|---|---|
| Compound | Method | time (min) | (%) | M/Z | Form |
| 1 | A | 2.60 | 100 | 399 | colorless solid |
| 2 | A | 2.35 | 100 | 331 | colorless solid |
| 3 | A | 2.58 | 100 | 399 | colorless solid |
| 4 | A | 2.45 | 96 | 365 | colorless solid |
| 5 | A | 0.68 | 100 | 332 | yellow solid |
| 6 | A | 1.17 | 100 | 332 | colorless solid |
| 7 | A | 1.83 | 100 | 366 | colorless solid |
| 8 | A | 2.55 | 98 | 385 | colorless solid |
| 9 | A | 2.34 | 100 | 365 | colorless solid |
| 10 | A | 1.93 | 100 | 356 | colorless solid |
| 11 | A | 2.51 | 100 | 399 | colorless solid |
| 12 | A | 3.40 | 100 | 516 | colorless solid |
| 13 | A | 3.28 | 100 | 482 | colorless solid |
| 14 | A | 3.14 | 99 | 434 | colorless solid |
| 15 | A | 3.26 | 98 | 462 | colorless solid |
| 16 | A | 2.17 | 100 | 449 | colorless solid |
| 17 | A | 1.93 | 100 | 449 | off-white solid |
| 18 | A | 3.87 | 100 | 463 | colorless solid |
| 19 | A | 3.24 | 100 | 463 | yellow solid |
| 20 | A | 6.96 | 100 | 376 | colorless oil |
| 21 | A | 7.36 | 100 | 404 | colorless oil |
| 22 | A | 7.13 | 100 | 419 | colorless oil |
| 23 | A | 6.85 | 100 | 391 | colorless solid |
| 24 | A | 2.58 | 100 | 399 | colorless solid |
| 25 | A | 2.50 | 100 | 419 | off-white solid |
| 26 | A | 2.02 | 100 | 317 | off-white solid |
| 27 | A | 2.13 | 98 | 331 | tan solid |
| 28 | A | 2.10 | 97 | 351 | colorless solid |
| 29 | A | 8.36 | 100 | 530 | colorless oil |
| 30 | A | 8.31 | 100 | 530 | colorless oil |
| 31 | A | 7.90 | 100 | 462 | colorless oil |
| 32 | A | 3.07 | 100 | 448 | colorless solid |
| 33 | A | 3.53 | 100 | 516 | colorless solid |
| 34 | A | 2.45 | 100 | 399 | colorless solid |
| 35 | A | 2.09 | 100 | 427 | colorless amorphous |
| 36 | A | 2.18 | 100 | 441 | tan solid |
| 37 | A | 2.16 | 100 | 387 | colorless oil |
| 38 | A | 2.08 | 99 | 401 | colorless oil |
| 39 | A | 2.19 | 100 | 401 | colorless oil |
| 40 | A | 2.02 | 100 | 456 | tan solid |
| 41 | A | 2.08 | 100 | 413 | tan solid |
| 42 | A | 2.54 | 100 | 503 | colorless solid |
| 43 | A | 2.02 | 96 | 426 | tan solid |
| 44 | A | 2.63 | 100 | 488 | tan solid |
| 45 | A | 2.23 | 98 | 454 | off-white solid |
| 46 | A | 1.99 | 98 | 456 | off-white solid |
| 47 | A | 2.70 | 100 | 487 | off-white solid |
| 48 | A | 2.85 | 100 | 501 | tan solid |
| 49 | A | 2.74 | 100 | 449 | ale-yellow solid |
| 50 | A | 8.82 | 100 | 468 | colorless solid |
| 51 | A | 7.68 | 100 | 412 | colorless solid |
| 52 | A | 6.13 | 100 | 350 | colorless solid |
| 53 | A | 7.37 | 100 | 437 | colorless solid |
| 54 | A | 5.42 | 97 | 416 | pale-yellow oil |
| 55 | A | 8.54 | 100 | 480 | colorless solid |
| 56 | A | 8.46 | 100 | 480 | pale-yellow solid |
| 57 | A | 8.39 | 100 | 480 | yellow solid |
| 58 | A | 8.54 | 100 | 496 | colorless solid |
| 59 | A | 8.57 | 100 | 496 | pale-yellow solid |
| 60 | A | 8.58 | 100 | 496 | colorless oil |
| 61 | A | 8.39 | 100 | 442 | colorless solid |
| 62 | A | 7.88 | 100 | 430 | colorless solid |
| 63 | A | 8.33 | 97 | 430 | colorless solid |
| 64 | A | 8.21 | 100 | 447 | colorless solid |
| 65 | A | 9.16 | 100 | 434 | pale-yellow solid |
| 66 | A | 8.89 | 100 | 468 | colorless solid |
| 67 | A | 8.50 | 100 | 400 | colorless solid |
| 68 | A | 6.21 | 100 | 401 | pale-yellow solid |
| 69 | A | 8.19 | 100 | 435 | pale-yellow solid |
| 70 | A | 5.57 | 100 | 401 | colorless solid |
| 71 | A | 4.13 | 100 | 401 | pale-yellow solid |
| 72 | A | 9.06 | 100 | 434 | colorless solid |
| 73 | A | 8.39 | 97 | 435 | colorless solid |
| 74 | A | 9.29 | 100 | 482 | colorless solid |
| 75 | A | 8.62 | 100 | 435 | pale-yellow solid |
| 76 | A | 8.65 | 100 | 431 | colorless solid |
| 77 | A | 9.21 | 100 | 425 | colorless solid |
| 78 | A | 8.31 | 100 | 468 | pale-yellow oil |
| 79 | A | 9.19 | 100 | 484 | pale-yellow solid |
| 80 | A | 8.69 | 100 | 430 | pale-yellow solid |
| 81 | A | 9.21 | 100 | 486 | pale-yellow oil |
| 82 | A | 10.08 | 97 | 406 | pale-yellow solid |
| 83 | A | 6.61 | 100 | 427 | off-white solid |
| 84 | A | 6.95 | 100 | 427 | colorless solid |
| 85 | A | 7.40 | 100 | 427 | colorless solid |
| 86 | A | 6.21 | 100 | 471 | yellow solid |
| 87 | A | 6.57 | 98 | 471 | pale-yellow oil |
| 88 | A | 6.57 | 98 | 471 | colorless oil |
| 89 | A | 2.67 | 100 | 484 | colorless solid |
| 90 | A | 2.74 | 100 | 470 | yellow solid |
| 91 | A | 8.84 | 100 | 456 | yellow solid |
| 92 | A | 9.15 | 100 | 470 | pale-yellow solid |
| 93 | A | 8.64 | 97 | 470 | colorless solid |
| 94 | A | 8.13 | 98 | 486 | pale-yellow solid |
| 95 | A | 8.87 | 100 | 482 | pale-yellow solid |
| 96 | A | 7.27 | 100 | 456 | colorless solid |
| 97 | A | 7.60 | 100 | 482 | pale-yellow solid |
| 98 | A | 7.21 | 100 | 498 | colorless solid |
| 99 | A | 7.40 | 100 | 470 | pale-yellow solid |
| 100 | A | 7.61 | 100 | 443 | colorless solid |
| 101 | A | 8.19 | 100 | 505 | colorless solid |
| 102 | A | 8.42 | 100 | 519 | colorless solid |
| 103 | A | 8.37 | 100 | 471 | colorless solid |
| 104 | A | 8.31 | 100 | 489 | pale-yellow solid |
| 105 | A | 4.76 | 98 | 429 | colorless solid |
| 106 | A | 5.93 | 100 | 441 | pale-yellow solid |
| 107 | A | 6.05 | 98 | 429 | pale-yellow solid |
| 108 | A | 8.42 | 100 | 471 | colorless solid |
| 109 | A | 9.05 | 100 | 462 | colorless solid |
| 110 | A | 8.46 | 100 | 459 | colorless solid |
| 111 | A | 8.49 | 100 | 459 | colorless solid |
| 112 | A | 5.75 | 100 | 445 | colorless solid |
| 113 | A | 5.64 | 100 | 445 | colorless solid |
| 114 | A | 8.46 | 95 | 436 | colorless solid |
| 115 | A | 9.68 | 95 | 434 | colorless solid |
| 116 | A | 9.45 | 100 | 420 | colorless solid |
| 117 | A | 7.13 | 100 | 430 | pale-yellow solid |
| 118 | A | 8.26 | 100 | 463 | colorless solid |
| 119 | A | 9.29 | 100 | 540 | colorless amorphous |
| 120 | A | 8.40 | 97 | 453 | colorless solid |
| 121 | A | 8.32 | 100 | 453 | colorless solid |
| 122 | A | 8.26 | 100 | 453 | colorless solid |
| 123 | A | 8.73 | 100 | 446 | colorless solid |
| 124 | A | 8.01 | 100 | 443 | colorless solid |
| 125 | A | 7.96 | 100 | 447 | colorless solid |
| 126 | A | 7.81 | 100 | 447 | colorless solid |
| 127 | A | 8.43 | 100 | 463 | colorless solid |
| 128 | A | 8.43 | 100 | 471 | colorless solid |
| 129 | A | 9.44 | 100 | 476 | colorless solid |
| 130 | A | 3.07 | 100 | 435 | colorless solid |
| 131 | A | 3.08 | 100 | 449 | colorless solid |
| 132 | A | 7.43 | 100 | 477 | colorless oil |
| 133 | A | 7.16 | 95 | 443 | colorless solid |
| 134 | A | 8.71 | 100 | 473 | colorless solid |
| 135 | A | 6.97 | 100 | 443 | pale-yellow solid |
| 136 | A | 3.07 | 100 | 435 | colorless solid |
| 137 | A | 3.06 | 98 | 449 | colorless solid |
| 138 | A | 7.21 | 100 | 477 | colorless solid |
| 139 | A | 8.17 | 100 | 436 | colorless solid |
| 140 | A | 6.65 | 100 | 459 | pale-yellow solid |
| 141 | A | 6.77 | 100 | 459 | pale-yellow solid |
| 142 | A | 3.04 | 100 | 432 | pale-yellow solid |
| 143 | A | 9.35 | 100 | 468 | colorless solid |
| 144 | A | 8.42 | 100 | 459 | colorless solid |
| 145 | A | 5.46 | 100 | 445 | colorless solid |
| 146 | A | 6.38 | 100 | 459 | pale-yellow solid |
| 147 | A | 9.14 | 95 | 493 | colorless solid |
| 148 | A | 8.46 | 100 | 459 | colorless solid |
| 149 | A | 7.63 | 100 | 479 | colorless solid |
| 150 | A | 5.69 | 100 | 445 | colorless solid |
| 151 | A | 6.50 | 100 | 459 | colorless solid |
| 152 | A | 7.71 | 100 | 493 | colorless solid |
| 153 | A | 6.33 | 100 | 383 | pale-yellow oil |
| 154 | A | 6.71 | 100 | 397 | colorless solid |
| 155 | A | 6.72 | 96 | 395 | colorless solid |
| 156 | A | 6.39 | 100 | 399 | colorless solid |
| 157 | A | 5.89 | 100 | 398 | colorless solid |
| 158 | A | 7.20 | 100 | 385 | pale-yellow oil |
| 159 | A | 4.75 | 100 | 366 | pale-yellow solid |
| 160 | A | 3.51 | 100 | 482 | colorless solid |
| 161 | A | 2.44 | 100 | 432 | pale-yellow solid |
| 162 | A | 3.14 | 100 | 468 | colorless solid |
| 163 | A | 7.71 | 96 | 489 | colorless solid |
| 164 | A | 7.64 | 100 | 491 | colorless solid |
| 165 | A | 5.77 | 100 | 443 | colorless solid |
| 166 | A | 6.43 | 100 | 398 | pale-yellow solid |
| 167 | A | 2.91 | 100 | 398 | pinkish solid |
| 168 | A | 5.54 | 100 | 402 | pale-yellow solid |
| 169 | A | 7.53 | 100 | 442 | pale-yellow solid |
| 170 | A | 8.05 | 100 | 507 | pale-yellow solid |
| 171 | A | 5.21 | 97 | 444 | yellow solid |
| 172 | A | 6.38 | 100 | 435 | colorless solid |
| 173 | A | 7.20 | 100 | 454 | colorless solid |
| 174 | A | 2.44 | 100 | 437 | colorless solid |
| 175 | A | 2.40 | 100 | 437 | colorless solid |
| 176 | A | 6.52 | 100 | 416 | yellow solid |
| 177 | A | 7.14 | 100 | 454 | pale-yellow solid |
| 178 | A | 6.96 | 100 | 454 | colorless solid |
| 179 | A | 2.84 | 100 | 459 | colorless solid |
| 180 | A | 7.69 | 100 | 492 | pale-yellow solid |
| 181 | A | 7.83 | 100 | 505 | colorless oil |
| 182 | A | 7.77 | 97 | 449 | pale-yellow solid |
| 183 | A | 7.45 | 100 | 492 | pale-yellow solid |
| 184 | A | 6.98 | 100 | 465 | colorless solid |
| 185 | A | 8.87 | 95 | 482 | colorless solid |
| 186 | A | 8.08 | 100 | 493 | colorless oil |
| 187 | A | 4.19 | 98 | 500 | colorless solid |
| 188 | A | 2.32 | 100 | 442 | pale-yellow solid |
| 189 | A | 3.83 | 95 | 509 | yellow solid |
| 190 | A | 8.34 | 97 | 553 | colorless amorphous |
| 191 | A | 6.58 | 100 | 483 | colorless amorphous |
| 192 | A | 8.03 | 100 | 503 | colorless solid |
| 193 | A | 4.79 | 100 | 495 | colorless solid |
| 194 | A | 3.01 | 100 | 445 | light-green solid |
| 195 | A | 8.55 | 100 | 504 | colorless amorphous |
| 196 | B | 10.11 | 100 | 387 | colorless solid |
| 197 | B | 10.17 | 100 | 435 | colorless solid |
| 198 | B | 10.05 | 100 | 399 | colorless solid |
| 199 | B | 9.89 | 99 | 394 | colorless solid |
| 200 | A | 8.24 | 100 | 489 | colorless solid |
| 201 | A | 7.20 | 100 | 438 | colorless solid |
| 202 | A | 7.25 | 97 | 436 | pale-yellow solid |
| 203 | A | 3.16 | 100 | 512 | colorless solid |
| 204 | A | 7.72 | 100 | 467 | colorless solid |
| 205 | B | 9.81 | 100 | 429 | colorless solid |
| 206 | B | 9.99 | 100 | 429 | colorless solid |
| 207 | B | 10.02 | 100 | 412 | colorless solid |
| 208 | B | 10.33 | 99 | 405 | colorless solid |
| 209 | B | 10.20 | 100 | 405 | colorless solid |
| 210 | B | 10.29 | 97 | 405 | colorless solid |
| 211 | B | 10.24 | 98 | 405 | colorless solid |
| 212 | A | 7.98 | 100 | 486 | colorless solid |
| 213 | A | 6.78 | 100 | 472 | colorless solid |
| 214 | A | 6.48 | 95 | 539 | colorless solid |
| 215 | A | 3.10 | 96 | 526 | colorless solid |
| 216 | A | 2.98 | 100 | 529 | pale-yellow solid |
| 217 | A | 7.50 | 98 | 467 | light-red solid |
| 218 | B | 9.82 | 100 | 442 | colorless solid |
| 219 | B | 9.91 | 99 | 443 | colorless solid |
| 220 | B | 9.09 | 100 | 428 | colorless solid |
| 221 | B | 9.15 | 99 | 429 | colorless solid |
| 222 | B | 10.49 | 99 | 438 | colorless solid |
| 223 | B | 9.74 | 100 | 456 | colorless solid |
| 224 | B | 11.33 | 100 | 527 | colorless solid |
| 225 | B | 11.41 | 100 | 571 | colorless solid |
| 226 | B | 10.09 | 100 | 469 | colorless solid |
| 227 | B | 10.34 | 100 | 513 | colorless oil |
| 228 | B | 11.31 | 95 | 525 | colorless solid |
| 229 | B | 10.62 | 96 | 481 | colorless solid |
| 230 | B | 11.15 | 93 | 470 | colorless solid |
| 231 | B | 11.62 | 97 | 512 | colorless solid |
| 232 | B | 10.97 | 96 | 522 | colorless solid |
| 233 | B | 10.84 | 95 | 522 | colorless solid |
| 234 | B | 10.83 | 96 | 509 | pale-yellow solid |
| 235 | B | 10.78 | 97 | 522 | colorless solid |
| 236 | B | 10.69 | 97 | 500 | colorless solid |
| 237 | B | 10.90 | 95 | 527 | colorless solid |
| 238 | B | 10.74 | 96 | 486 | colorless solid |
| 239 | A | 5.86 | 100 | 471 | colorless solid |
| 240 | A | 6.61 | 100 | 485 | colorless solid |
| 241 | A | 6.76 | 100 | 499 | colorless solid |
| 242 | B | 10.90 | 100 | 475 | colorless solid |
| 243 | B | 11.09 | 95 | 492 | colorless solid |
| 244 | B | 11.11 | 98 | 492 | colorless solid |
| 245 | B | 11.03 | 100 | 492 | colorless solid |
| 246 | B | 10.93 | 100 | 474 | colorless solid |
| 247 | B | 11.92 | 100 | 452 | colorless solid |
| 248 | B | 11.40 | 100 | 364 | colorless solid |
| 249 | B | 10.85 | 100 | 392 | colorless solid |
| 250 | B | 10.65 | 100 | 376 | colorless solid |
| 251 | B | 9.76 | 100 | 507 | colorless solid |
| 252 | B | 10.91 | 97 | 462 | colorless solid |
| 253 | B | 9.54 | 99 | 423 | colorless solid |
| 254 | B | 8.78 | 100 | 460 | colorless solid |
| 255 | B | 9.46 | 100 | 420 | colorless solid |
| 256 | B | 9.77 | 100 | 481 | colorless solid |
| 257 | B | 9.85 | 100 | 499 | colorless solid |
| 258 | B | 9.92 | 100 | 516 | colorless solid |
| 259 | B | 11.03 | 98 | 492 | colorless solid |
| 260 | B | 9.96 | 98 | 486 | colorless solid |
| 261 | B | 9.89 | 100 | 514 | colorless solid |
| 262 | B | 8.48 | 100 | 474 | colorless solid |
| 263 | A | 7.28 | 100 | 469 | pale-yellow solid |
| 264 | A | 2.92 | 100 | 468 | colorless solid |
| 265 | A | 7.75 | 100 | 472 | colorless solid |
| 266 | A | 4.38 | 98 | 486 | colorless solid |
| 267 | B | 9.76 | 100 | 481 | colorless solid |
| 268 | B | 9.72 | 100 | 516 | colorless solid |
| 269 | A | 6.72 | 100 | 530 | colorless solid |
| 270 | A | 5.65 | 98 | 530 | colorless solid |
| 271 | A | 7.54 | 100 | 530 | colorless amorphous |
| 272 | A | 7.40 | 100 | 444 | colorless solid |
| 273 | A | 7.72 | 100 | 497 | colorless oil |
| 274 | A | 6.60 | 100 | 465 | pale-yellow |
| | | | | | amorhous |
| 275 | A | 7.74 | 100 | 493 | orange amorhous |
| 276 | B | 10.17 | 100 | 426 | colorless oil |
| 277 | B | 10.91 | 100 | 418 | colorless solid |
| 278 | A | 7.26 | 100 | 435 | colorless solid |
| 279 | B | 9.96 | 100 | 413 | colorless solid |
| 280 | B | 9.60 | 100 | 437 | colorless solid |
| 281 | B | 8.89 | 100 | 431 | colorless solid |
| 282 | B | 8.75 | 100 | 394 | colorless solid |
| 283 | B | 8.58 | 98 | 420 | colorless solid |
| 284 | B | 9.62 | 98 | 437 | colorless solid |
| 285 | B | 10.44 | 100 | 524 | colorless oil |
| 286 | B | 10.08 | 100 | 514 | colorless solid |
| 287 | B | 9.93 | 100 | 462 | colorless solid |
| 288 | B | 9.65 | 100 | 457 | colorless solid |
| 289 | B | 10.47 | 100 | 540 | colorless oil |
| 290 | B | 10.52 | 98 | 540 | colorless oil |
| 291 | B | 9.40 | 100 | 457 | colorless solid |
| 292 | B | 10.41 | 100 | 490 | colorless solid |
| 293 | B | 8.55 | 97 | 438 | pale-yellow solid |
| 294 | B | 11.21 | 100 | 432 | colorless solid |
| 295 | A | 7.14 | 100 | 553 | colorless solid |
| 296 | A | 7.18 | 100 | 553 | colorless solid |
| 297 | B | 5.27 | 99 | 409 | colorless solid |
| 298 | B | 5.97 | 100 | 418 | colorless solid |
| 299 | B | 5.25 | 100 | 409 | colorless solid |
| 300 | B | 5.02 | 100 | 435 | colorless solid |
| 301 | B | 5.39 | 100 | 402 | colorless solid |
| 302 | A | 3.14 | 100 | 552 | colorless amorphous |
| 303 | A | 8.27 | 100 | 525 | colorless oil |
| 304 | A | 8.39 | 100 | 577 | colorless amorphous |
| 305 | A | 4.33 | 100 | 469 | colorless solid |
| 306 | A | 9.51 | 100 | 486 | colorless oil |
| 307 | A | 8.75 | 100 | 536 | colorless solid |
| 308 | A | 6.47 | 100 | 503 | pale-yellow solid |
| 309 | B | 10.58 | 100 | 404 | colorless solid |
| 310 | B | 3.35 | 100 | 426 | colorless solid |
| 311 | A | 8.06 | 100 | 504 | colorless oil |
| 312 | A | 7.86 | 100 | 504 | colorless solid |
| 313 | A | 6.27 | 99 | 505 | colorless oil |
| 314 | A | 5.49 | 100 | 539 | colorless solid |
| 315 | A | 5.09 | 100 | 546 | colorless solid |
| 316 | A | 6.69 | 98 | 454 | colorless solid |
| 317 | A | 4.87 | 100 | 411 | pale-yellow solid |
| 318 | A | 6.57 | 100 | 420 | pale-yellow solid |
| 319 | A | 7.78 | 100 | 482 | colorless solid |
| 320 | A | 8.37 | 100 | 518 | pale-yellow solid |
| 321 | A | 7.67 | 100 | 468 | yellow solid |
| 322 | A | 8.49 | 97 | 504 | pale-yellow solid |
| 323 | A | 6.77 | 97 | 417 | pale-yellow solid |
| 324 | A | 8.28 | 98 | 486 | pale-yellow oil |
| 325 | B | 8.66 | 100 | 397 | colorless solid |
| 326 | B | 8.70 | 97 | 411 | colorless oil |
| 327 | B | 10.01 | 100 | 541 | colorless oil |
| 328 | B | 10.18 | 100 | 585 | colorless oil |
| 329 | B | 9.97 | 100 | 555 | colorless oil |
| 330 | B | 10.13 | 100 | 599 | colorless oil |
| 331 | B | 9.61 | 95 | 467 | colorless oil |
| 332 | B | 9.75 | 100 | 511 | pale-yellow solid |
| 333 | B | 11.95 | 100 | 452 | colorless solid |
| 334 | C | 2.75 | 100 | 404 | colorless solid |
| 335 | C | 2.26 | 100 | 371 | off-white solid |
| 336 | C | 2.17 | 100 | 474 | colorless solid |
| 337 | C | 2.98 | 100 | 543 | off-white solid |
| 338 | C | 2.92 | 100 | 527 | off-white solid |
| 339 | C | 2.76 | 100 | 379 | off-white solid |
| 340 | C | 2.80 | 100 | 438 | off-white solid |
| 341 | C | 2.84 | 100 | 454 | off-white solid |
| 342 | C | 2.84 | 100 | 379 | off-white solid |
| 343 | C | 3.17 | 100 | 413 | colorless solid |
| 344 | C | 2.09 | 100 | 429 | colorless solid |
| 345 | C | 2.72 | 100 | 404 | colorless solid |
| 346 | C | 3.06 | 100 | 396 | colorless solid |
| 347 | C | 2.23 | 100 | 396 | colorless solid |
| 348 | C | 2.54 | 100 | 395 | off-white solid |
| 349 | C | 2.53 | 100 | 395 | off-white solid |
| 350 | C | 3.24 | 100 | 490 | colorless solid |
| 351 | C | 3.17 | 100 | 413 | colorless solid |
| 352 | C | 2.62 | 100 | 388 | colorless solid |
| 353 | C | 2.64 | 100 | 388 | colorless solid |
| 354 | C | 2.79 | 100 | 454 | off-white solid |
| 355 | C | 2.49 | 100 | 421 | off-white solid |
| 356 | C | 2.33 | 100 | 371 | off-white solid |
| 357 | C | 2.97 | 100 | 463 | off-white solid |
| 358 | C | 2.96 | 100 | 463 | off-white solid |
| 359 | C | 3.19 | 100 | 446 | colorless solid |
| 360 | B | 10.91 | 100 | 380 | colorless solid |
| 361 | B | 11.16 | 100 | 394 | colorless solid |
| 362 | B | 10.68 | 100 | 364 | colorless solid |
| 363 | B | 10.97 | 100 | 378 | colorless solid |
| 364 | B | 10.93 | 100 | 390 | colorless solid |
| 365 | B | 10.73 | 98 | 364 | colorless solid |
| 366 | B | 10.46 | 100 | 392 | colorless solid |
| 367 | B | 10.85 | 100 | 406 | colorless oil |
| 368 | B | 10.25 | 100 | 438 | colorless solid |
| 369 | B | 11.15 | 100 | 420 | colorless oil |
| 370 | C | 2.88 | 99 | 513 | off-white solid |
| 371 | C | 2.81 | 100 | 499 | off-white solid |
| 372 | C | 2.87 | 100 | 463 | off-white solid |
| 373 | C | 3.01 | 100 | 463 | pale-yellow solid |
| 374 | C | 2.68 | 99 | 463 | off-white solid |
| 375 | C | 2.97 | 100 | 463 | off-white solid |
| 376 | C | 2.88 | 98 | 463 | off-white solid |
| 377 | C | 2.83 | 100 | 454 | off-white solid |

Compounds of the invention have been tested in the calcium mobilization and/or electrophysiological assay for N-type calcium channel blocking activity, which are described in detail above. Some compounds described have also been tested in the calcium mobilization assay for L-type calcium channel blocking activity, which is described in detail above. Representative values are presented in TABLE 6.

TABLE 6
Evaluation of the tested compounds as N-type calcium channel
(NTCC) blockers and L-type calcium channel (LTCC) blockers
after a calcium mobilization in vitro assay

| EXAMPLE | NTCC IC₅₀(nM) | LTCC IC₅₀(nM) |
|---|---|---|
| 4 | 860 | 15200 |
| 8 | 690 | 9600 |
| 12 | 820 | >20000 |
| 16 | 260 | 6600 |
| 19 | 760 | 4900 |
| 24 | 990 | 7200 |
| 50 | 430 | >20000 |
| 30 | 330 | >20000 |
| 77 | 220 | >20000 |
| 182 | 690 | >20000 |
| 227 | 1100 | Not determined |
| 277 | 610 | >20000 |
| 295 | 160 | >20000 |
| 308 | 360 | >20000 |
| 328 | 1100 | Not determined |
| 369 | 80 | >20000 |

The following compounds also exhibited an NTCC IC₅₀ of about 1.0 μM or less. 29, 31, 47, 48, 51, 53, 56, 58, 59, 62, 63, 64, 65, 67, 68, 69, 70, 71, 72, 73, 74, 75, 76, 78, 79, 80, 81, 82, 85, 91, 93, 94, 95, 99, 101, 108, 110, 111, 118, 119, 120, 121, 127, 128, 134, 144, 147, 152, 160, 162, 163, 164, 173, 177, 178, 181, 186, 189, 191, 196, 197, 199, 210, 214, 216, 228, 229, 231, 232, 237, 238, 243, 244, 251, 252, 256, 263, 265, 267, 268, 269, 270, 271, 273, 274, 275, 276, 278, 280, 281, 282, 284, 285, 286, 287, 288, 289, 290, 291, 292, 294, 296, 302, 303, 304, 306, 307, 309, 310, 311, 313, 314, 315, 321, 329, 366, 367 and 368.

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. All patents and publications cited herein are fully incorporated by reference herein in their entirety.

What is claimed is:

1. A compound having the Formula I: 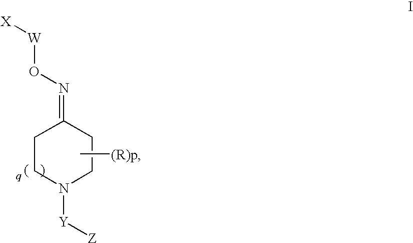
a pharmaceutically acceptable salt, a prodrug or a solvate thereof, wherein:
W is absent, optionally substituted lower alkylene or optionally substituted lower alkenylene,
X is optionally substituted aryl, optionally substituted heterocyclyl, optionally substituted cycloalkyl or optionally substituted cycloalkenyl and
When W is optionally substituted lower alkylene or optionally substituted lower alkenylene, then X can additionally be hydrogen;
Y is CO, SOₘ or CR³R⁴;
Z is optionally substituted lower alkyl, optionally substituted lower alkenyl, optionally substituted lower alkynyl, optionally substituted lower alkoxy, optionally substituted lower alkenyloxy, optionally substituted lower alkynyloxy, optionally substituted amino, optionally substituted lower alkylthio, optionally substituted lower alkenylthio, optionally substituted lower alkynylthio, optionally substituted cycloalkyl, optionally substituted cycloalkenyl, optionally substituted aryl or optionally substituted heterocyclyl,
when Y is CO, then Z can additionally be optionally substituted acyl, optionally substituted carbamoyl, optionally substituted cycloalkyloxy, optionally substituted cycloalkenyloxy, optionally substituted aryloxy or optionally substituted heterocyclyloxy,
when Y is CR³R⁴, then Z can additionally be hydrogen, hydroxy, halogen, carboxy, optionally substituted lower alkoxycarbonyl, optionally substituted lower alkylthio, optionally substituted acyl, optionally substituted acyloxy, optionally substituted lower alkylsulfonyl, optionally substituted lower alkylsulfonyloxy, optionally substituted lower alkylsulfinyl, optionally substituted carbamoyl, optionally substituted carbamoyloxy, optionally substituted sulfamoyl, optionally substituted sulfamoyloxy, optionally substituted aryloxy, optionally substituted aryloxycarbonyl, optionally substituted arylthio, optionally substituted arylsulfonyl, optionally substituted arylsulfinyl, optionally substituted arylsulfonyloxy, optionally substituted arylsulfinyloxy, optionally substituted heterocyclyloxy, optionally substituted heterocyclyloxycarbonyl, optionally substituted heterocyclylthio, optionally substituted heterocyclylsulfonyl, optionally substituted heterocyclylsulfinyl, optionally substituted heterocyclylsulfonyloxy, optionally substituted heterocyclylsulfinyloxy,
R³ and R⁴ are each independently hydrogen, halogen, lower alkyl, halo(lower)alkyl, hydroxy(lower)alkyl, amino, lower alkylamino, optionally substituted cycloalkyl, optionally substituted cycloalkenyl, optionally substituted aryl or optionally substituted heterocyclyl, or together with the carbon atom to which they are attached form optionally substituted carbocycle or optionally substituted heterocycle;
R is lower alkyl, hydroxy(lower)alkyl, lower alkoxy(lower)alkyl, carboxy, lower alkoxycarbonyl, carbamoyl or lower alkylcarbamoyl,
m is 1 or 2,
p is 0, 1 or 2, and
q is 0, 1 or 2 with the following provisos:
i) when Y is SO₂, then Z is not lower alkyl substituted with at least one substituent selected from CONHOH, COOH and lower alkoxycarbonyl,
ii) Y-Z is not benzoyl, acetyl, carbamoyl or lower alkoxycarbonyl,
iii) when Y is CO, then Z is not methylene substituted with heterocyclidene, and
iv) when Y is SO₂, then —W—X is not 2-tetrahydrofuryl.

2. The compound of claim 1, wherein q is 1.

3. The compound of claim 1 or 2, wherein W is (CR¹R²)n, n is 0, 1, 2 or 3, R¹ and R² are each independently hydrogen, halogen, lower alkyl, halo(lower)alkyl, hydroxy(lower)alkyl, lower alkoxy(lower)alkyl, optionally substituted aryl(lower)alkyl, optionally substituted aryloxy(lower)alkyl, optionally substituted heterocyclyl(lower)alkyl, optionally substituted heterocyclyloxy(lower)alkyl, optionally substituted cycloalkyl, optionally substituted cycloalkenyl, optionally substituted aryl or optionally substituted heterocyclyl and X is optionally substituted aryl, optionally substituted heterocyclyl, optionally substituted cycloalkyl or optionally substituted cycloalkenyl.

4. The compound of any of claims 1 to 3, wherein W is optionally substituted methylene.

5. The compound of any of claims 1 to 4, wherein X is optionally substituted phenyl or optionally substituted pyridyl.

6. The compound of any of claims 1 to 5, wherein Y is CO or SO₂.

7. The compound of any of claims 1 to 6, wherein Z is optionally substituted lower alkyl or optionally substituted phenyl.

8. A pharmaceutical composition, comprising the compound of any of claims 1 to 7 and a pharmaceutically acceptable carrier.

9. A method of treating, preventing or ameliorating a disorder responsive to the blockade of calcium channels in a mammal suffering from said disorder, comprising administering to a mammal in need of such treatment, prevention or amelioration an effective amount of a compound of any of claims 1 to 7.

10. The method of claim 9, wherein a disorder responsive to the blockade of N-type calcium channels is treated, prevented or ameliorated.

11. A method for treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal, comprising administering an effective amount of a compound of any of claims 1 to 7.

12. The method of claim 11, wherein the method is for treating, preventing or ameliorating pain selected from chronic pain, acute pain, and surgical pain.

13. A method of modulating calcium channels in a mammal, comprising administering to the mammal at least one compound of any one of claims 1 to 7.

14. The method of claim 13, wherein the N-type calcium channel is modulated.

15. A compound having the Formula I as claimed in claims 1 to 7, wherein the compound is ³H, ¹¹C, or ¹⁴C radiolabeled.

16. A method of screening a candidate compound for the ability to bind to a receptor using a radiolabeled compound of claim 15, comprising a) introducing a fixed concentration of the radiolabeled compound to the receptor to form a mixture; b) titrating the mixture with a candidate compound; and c) determining the binding of the candidate compound to said receptor.

17. Use of a compound of Formula I as claimed in any one of claims 1 to 7 in the manufacture of a medicament for treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal.

18. Use of a compound of Formula I as claimed in any one of claims 1 to 7 in the manufacture of a medicament for treating, preventing or ameliorating pain selected from chronic pain, acute pain, and surgical pain.

19. A pharmaceutical composition for modulating calcium channels in a mammal, comprising the compound having the Formula I′: 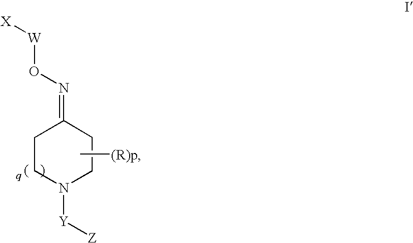
a pharmaceutically acceptable salt, a prodrug or a solvate thereof, wherein:
W is absent, optionally substituted lower alkylene or optionally substituted lower alkenylene,
X is optionally substituted aryl, optionally substituted heterocyclyl, optionally substituted cycloalkyl or optionally substituted cycloalkenyl and
When W is optionally substituted lower alkylene or optionally substituted lower alkenylene, then X can additionally be hydrogen;
Y is CO, SOₘ or CR³R⁴;
Z is optionally substituted lower alkyl, optionally substituted lower alkenyl, optionally substituted lower alkynyl, optionally substituted lower alkoxy, optionally substituted lower alkenyloxy, optionally substituted lower alkynyloxy, optionally substituted amino, optionally substituted lower alkylthio, optionally substituted lower alkenylthio, optionally substituted lower alkynylthio, optionally substituted cycloalkyl, optionally substituted cycloalkenyl, optionally substituted aryl or optionally substituted heterocyclyl,
when Y is CO, then Z can additionally be optionally substituted acyl, optionally substituted carbamoyl, optionally substituted cycloalkyloxy, optionally substituted cycloalkenyloxy, optionally substituted aryloxy or optionally substituted heterocyclyloxy,
when Y is CR³R⁴, then Z can additionally be hydrogen, hydroxy, halogen, carboxy, optionally substituted lower alkoxycarbonyl, optionally substituted lower alkylthio, optionally substituted acyl, optionally substituted acyloxy, optionally substituted lower alkylsulfonyl, optionally substituted lower alkylsulfonyloxy, optionally substituted lower alkylsulfinyl, optionally substituted carbamoyl, optionally substituted carbamoyloxy, optionally substituted sulfamoyl, optionally substituted sulfamoyloxy,
optionally substituted aryloxy, optionally substituted aryloxycarbonyl, optionally substituted arylthio, optionally substituted arylsulfonyl, optionally substituted arylsulfinyl, optionally substituted arylsulfonyloxy, optionally substituted arylsulfinyloxy, optionally substituted heterocyclyloxy, optionally substituted heterocyclyloxycarbonyl, optionally substituted heterocyclylthio, optionally substituted heterocyclylsulfonyl, optionally substituted heterocyclylsulfinyl, optionally substituted heterocyclylsulfonyloxy, optionally substituted heterocyclylsulfinyloxy,
when Z is optionally substituted cycloalkyl, optionally substituted cycloalkenyl, optionally substituted aryl or optionally substituted heterocyclyl, then Y can additionally be absent;
R³ and R⁴ are each independently hydrogen, halogen, lower alkyl, halo(lower)alkyl, hydroxy(lower)alkyl, amino, lower alkylamino, optionally substituted cycloalkyl, optionally substituted cycloalkenyl, optionally substituted aryl or optionally substituted heterocyclyl, or together with the carbon atom to which they are attached form optionally substituted carbocycle or optionally substituted heterocycle;
R is lower alkyl, hydroxy(lower)alkyl, lower alkoxy(lower)alkyl, carboxy, lower alkoxycarbonyl, carbamoyl or lower alkylcarbamoyl,
m is 1 or 2,
p is 0, 1 or 2, and
q is 0, 1 or 2
and a pharmaceutically acceptable carrier.

20. The pharmaceutical composition of claim 19, wherein q is 1.

21. The pharmaceutical composition of claim 19 or 20, wherein W is (CR¹R²)n, n is 0, 1, 2 or 3, R¹ and R² are each independently hydrogen, halogen, lower alkyl, halo(lower)alkyl, hydroxy(lower)alkyl, lower alkoxy(lower)alkyl, optionally substituted aryl(lower)alkyl, optionally substituted aryloxy(lower)alkyl, optionally substituted heterocyclyl(lower)alkyl, optionally substituted heterocyclyloxy(lower)alkyl, optionally substituted cycloalkyl, optionally substituted cycloalkenyl, optionally substituted aryl or optionally substituted heterocyclyl and X is optionally substituted aryl, optionally substituted heterocyclyl, optionally substituted cycloalkyl or optionally substituted cycloalkenyl.

22. A method of treating, preventing or ameliorating a disorder responsive to the blockade of calcium channels in a mammal suffering from said disorder, comprising administering to a mammal in need of such treatment, prevention or amelioration an effective amount of a compound having the Formula I′ described in claim 19.

23. A method for treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal, comprising administering an effective amount of a compound having the Formula I′ described in claim 19.

24. The method of claim 23, wherein the method is for treating, preventing or ameliorating pain selected from chronic pain, acute pain, and surgical pain.

25. A method of modulating calcium channels in a mammal, comprising administering to the mammal at least one compound having the Formula I′ described in claim 19.

26. A method of screening a candidate compound for the ability to bind to a receptor using a ³H, ¹¹C, or ¹⁴C radiolabeled compound having the Formula I′ described in claim 19, comprising a) introducing a fixed concentration of the radiolabeled compound to the receptor to form a mixture; b) titrating the mixture with a candidate compound; and c) determining the binding of the candidate compound to said receptor.

27. Use of a compound having the Formula I′ described in claim 19 in the manufacture of a medicament for the treating, preventing or ameliorating stroke, neuronal damage resulting from head trauma, epilepsy, pain, migraine, a mood disorder, schizophrenia, a neurodegenerative disorder, depression, anxiety, a psychosis, hypertension or cardiac arrhythmia in a mammal.

28. Use of a compound having the Formula I′ described in claim 19 in the manufacture of a medicament for the treating, preventing or ameliorating pain selected from chronic pain, acute pain, and surgical pain.
